(19) United States
(12) Patent Application Publication
(10) Pub. No.: US 2024/0208912 A1
(43) Pub. Date: Jun. 27, 2024

(54) MTA-Cooperative PRMT5 Inhibitors

(71) Applicant: Mirati Therapeutics, Inc., San Diego, CA (US)

(72) Inventors: Thomas P. Bobinski, San Diego, CA (US); Christopher Ronald Smith, San Diego, CA (US); Matthew Arnold Marx, San Diego, CA (US); John Michael Ketcham, San Diego, CA (US); Aaron Craig Burns, San Diego, CA (US); John David Lawson, Carlsbad, CA (US); Svitlana Kulyk, Redwood City, CA (US); Jon Kuehler, San Diego, CA (US); Anthony Ivetac, San Diego, CA (US)

(21) Appl. No.: 18/549,864

(22) PCT Filed: Mar. 11, 2022
(86) PCT No.: PCT/US2022/020056

§ 371 Date: Sep. 8, 2023

Related U.S. Application Data
(60) Provisional application No. 63/200,521, filed on Mar. 11, 2021.
(60) Provisional application No. 63/163,002, filed on Mar. 18, 2021.
(60) Provisional application No. 63/297,537, filed on Jan. 7, 2022.

(51) Int. Cl. C07D 237/34; C07D 401/04; C07D 401/14; C07D 403/04; C07D 403/14; C07D 405/04; C07D 405/14; C07D 409/14; C07D 413/04; C07D 417/04; C07D 471/04; C07D 487/04; C07D 495/04; C07D 498/04; C07D 513/04

(52) U.S. Cl. CPC C07D 237/34; C07D 401/04; C07D 401/14; C07D 403/04; C07D 403/14; C07D 405/04; C07D 405/14; C07D 409/14; C07D 413/04; C07D 417/04; C07D 471/04; C07D 487/04; C07D 495/04; C07D 498/04; C07D 513/04

(57) ABSTRACT

Disclosed are compounds of Formula IIA, IA-1, IIB, IIB-1, IIC and IIC-1: and pharmaceutical compositions and methods of use thereof These compounds inhibit Protein Arginine M-Methyl Transferase 5 (PRMT5) activity and are useful in methods and pharmaceutical compositions for treating cancer.

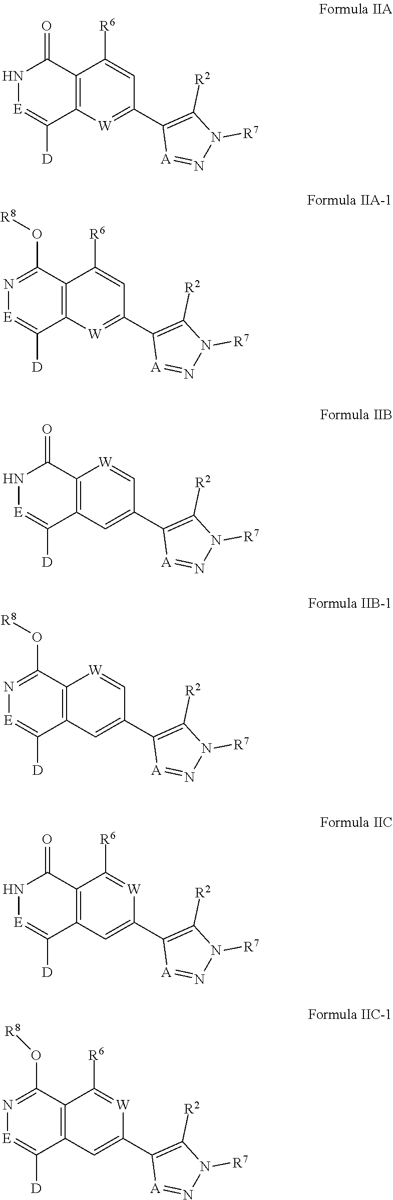

### CROSS-REFERENCE TO RELATED APPLICATIONS

This application claims the benefit of priority of U.S. Provisional Application No. 63/200,521, filed Mar. 11, 2021, U.S. Provisional Application No. 63/163,002, filed Mar. 18, 2021, and U.S. Provisional Application No. 63/297,537, filed Jan. 7, 2022, the disclosure of each of which is hereby incorporated by reference in its entirety.

### FIELD OF THE INVENTION

The present invention relates to compounds that are MTA-cooperative inhibitors of Protein Arginine N-Methyl Transferase 5 (PRMT5). In particular, the present invention relates to compounds, pharmaceutical compositions comprising the compounds and methods for use therefor.

### BACKGROUND OF THE INVENTION

Protein Arginine N-Methyl Transferase (PRMT5) is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recognition and orientation and is also required for PRMT5-catalyzed histone 2A and histone 4 methyltransferase activity (e.g., see Ho et al., (2013) PLOS ONE 8(8): 10.1371/annotation/e6b5348e-9052-44ab-8f06-90d01dc88fc2).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139(39):13754-13760. doi: 10.1021/jacs.7b05803. Epub 2017 Sep. 20).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

### SUMMARY OF THE INVENTION

Thus, we realized that MTA-cooperative inhibition of PRMT5 activity in MTAP deleted cancers will provide therapeutic benefit for a wide range of cancers. The compounds of the present invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

There is a need to develop new MTA-cooperative PRMT5 inhibitors that are capable of inhibiting PRMT5 activity in the presence of elevated MTA concentrations, particularly in MTAP-deficient cells.

In one aspect of the invention, compounds are provided that are represented by compounds of Formula IIA, IIA-1, IIB, IIB-1, IIC or IIC-1:

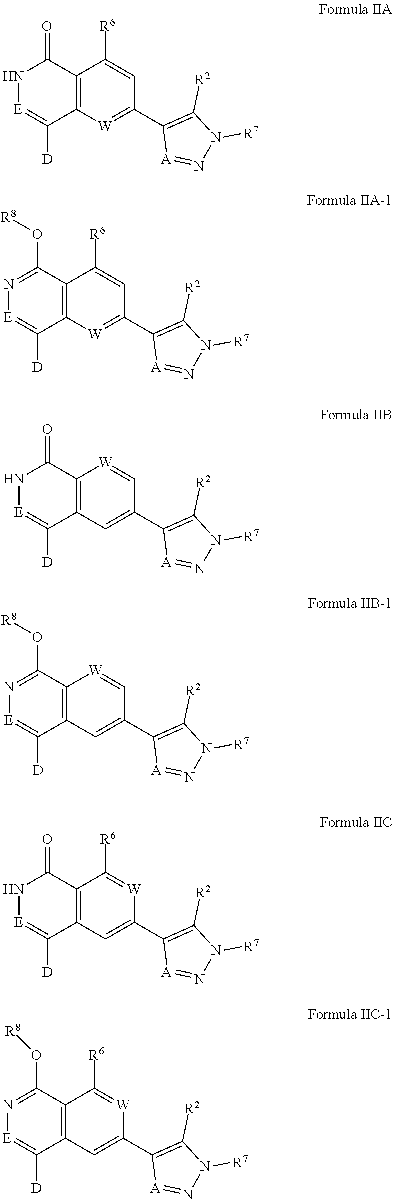

or a pharmaceutically acceptable salt thereof, wherein:
A is CR⁹ or N;
D is —CO₂R³⁰, (C(R⁹)₂)₁₋₂—NHR¹¹,

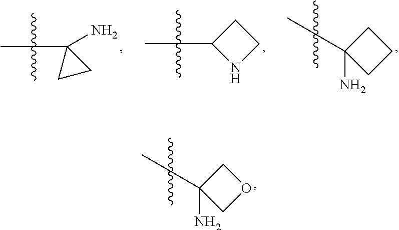 [SMILES: *C1(N)CC1 ; *C1CCN1 ; *C1(N)CCC1 ; *C1(N)COC1]

or D is

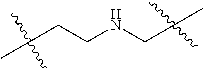 [SMILES: *CCNC*]

where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵, or;
each R⁴ is independently hydrogen or C1-C3 alkyl;
each R⁵ is independently cyano, oxo, halogen, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl;
each R⁹ is independently H or C1-C3 alkyl, halogen or haloalkyl, R³⁰ is hydrogen or C1-C6 alkyl;
R¹⁰ is H, C1-C3 alkyl, halogen, haloalkyl, or C3-C7 heterocycloalkyl; and
R¹¹ represents hydrogen or —C(O)R², where R² is hydrogen or C1-C3 alkyl.

In another aspect of the invention, intermediates are provided that are useful for the preparation of compounds of Formulas HA, IIA-1, IB, IIB-1, IC, IIC-1, IVA, IVA-1, IVB, IVB-1, IVC and IVC-1.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting PRMT5 activity in a in a cell, comprising contacting the cell with a compound of Formula IIA, IIA-1, IIB, 1B-1, IIC, IIC-1, IVA, IVA-1, IVB, IVB-1, IVC and IVC-1. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula IIA, IIA-1, IIB, IIB-1, IIC, IIC-1, IVA, IVA-1, IVB, IVB-1, IVC and IVC-1 or a pharmaceutically acceptable salt thereof, or a pharmaceutical composition thereof as defined herein. In one embodiment, the cell is an MTAP-deficient cell.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method for treating cancer in a patient in need thereof, the method comprising (a) determining that the cancer is associated with MTAP double deletion (e.g., an MTAP-associated cancer); and (b) administering to the patient a therapeutically effective amount of a compound of Formula IIA, IIA-1, IIB, IIB-1, IIC, IIC-1, IVA, IVA-1, IVB, IVB-1, IVC and IVC-1 or a pharmaceutically acceptable salt thereof, or a pharmaceutical composition thereof.

### DETAILED DESCRIPTION OF THE INVENTION

The present invention relates to MTA-cooperative PRMT5 inhibitors. In particular, the present invention relates to compounds that inhibit PRMT5 activity in the presence of bound MTA, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S).

As used herein, “PRMT5” refers to a mammalian Protein Arginine N-Methyl Transferase 5 (PRMT5) enzyme.

As used herein, a “PRMT5 inhibitor” or “MTA-cooperative PRMT5 inhibitor” refers to compounds of the present invention that are represented by Formula (I) or Formula (II) (including Formula IIA, IIA-1, IIB-1, IIC and/or IIC-1) or Formula (IV) (including Formula IVA, IVA-1, IVB, IVB-1, IVC and IVC-1) as described herein. These compounds are capable of negatively modulating or inhibiting all or a portion of the enzymatic activity of the PRMT5 in the presence of bound MTA in vitro or in vivo, or in cells expressing elevated levels of MTA.

As used herein, “MTAP” refers to a mammalian methylthioadenosine phosphorylase (MTAP) enzyme.

An “MTAP-associated disease or disorder” as used herein refers to diseases or disorders associated with or mediated by or having a loss of MTAP activity resulting in sensitizing the disorder to selective inhibition of PRMT5 activity. A non-limiting example of an MTAP-associated disease or disorder is an MTAP-associated cancer.

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC1-C6 alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NR^{x}, wherein R^{x} is hydrogen or C1-C3 alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C1-C6)alkyl(C6-C10)aryl, including, without limitation, benzyl, phenethyl, and naphthylmethyl. For example, an arC1-C3alkyl is an aryl group covalently linked to a C1-C3 alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, THFyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via an alkylene linker

As used herein, the term “heteroaryl” refers to a group having 5 to 14 ring atoms, preferably 5, 6, 10, 13 or 14 ring atoms; having 6, 10, or 14π electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom.

Examples of heteroaryl groups include acridinyl, azocinyl, benzimidazolyl, benzofuranyl, benzo[d]oxazol-2 (3H)-one, 2H-benzo[b][1,4]oxazin-3 (4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaralkyl” or “L-heteroarylalkyl” group comprises a heteroaryl group covalently linked to another group via an alkylene linker. Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

As used herein, “an effective amount” of a compound is an amount that is sufficient to negatively modulate or inhibit the activity of PRMT5 enzyme.

As used herein, a “therapeutically effective amount” of a compound is an amount that is sufficient to ameliorate or in some manner reduce a symptom or stop or reverse progression of a condition, or negatively modulate or inhibit the activity of PRMT5. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect of the invention, compounds are provided that are represented by Formula

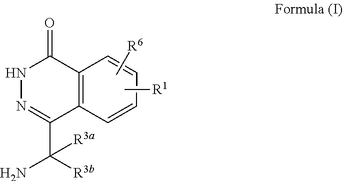

or a pharmaceutically acceptable salt thereof:
wherein:
R¹ is hydrogen, halogen, hydroxyalkyl, -L-CN, —Y—C1-C5 alkyl, —Y-cycloalkyl, —Y-heterocyclyl, —Y-aryl, —Y-arC1-C3alkyl or —Y-heteroaryl, wherein the cycloalkyl, the heterocyclyl, the aryl, and the heteroaryl portions are each optionally substituted with one or more R²;
each Y is a bond or —NR⁴—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵;
each X is independently a bond, O, S, —NR⁴— or —NR⁴C(O)—;
each Z is independently a bond, —SO—, —SO₂—, —CH(OH)— or —C(O)—;
each L is independently a bond or C1-C3 alkylene;
R^{3a} and R^{3b} are each independently hydrogen or deuterium, or R^{3a} and R^{3b} together are oxo;
each R⁴ is independently hydrogen or C1-C3 alkyl;
each R⁵ is independently cyano, oxo, halogen, C1-C3 alkyl, hydroxyalkyl, alkoxy, —X— haloalkyl, —Z-cycloalkyl, —X-arC1-C3alkyl, X-arC1-C3alkyl substituted with cyano, —X-L-cycloalkyl, —X-L-heteroaryl optionally substituted with one or more C1-C3alkyl or oxo, or —X-aryl; and
R⁶ is hydrogen, halogen, haloalkyl, C1-C3 alkyl or alkoxy.

In one embodiment for compounds of Formula (I), R¹ is hydrogen.

In another embodiment for compounds of Formula (I), R¹ is halogen. In certain embodiments, the halogen is bromine.

In one embodiment for compounds of Formula (I), R¹ is -L-CN. In one embodiment, L is C1-C3 alkylene. In certain embodiments, the C1-C3 alkylene is methylene.

In one embodiment for compounds of Formula (I), R¹ is —Y—C1-C5 alkyl. In one embodiment, Y is a bond and the C1-C5 alkyl is methyl. In one embodiment, Y is —NR⁴— and the C1-C5 alkyl is methyl, ethyl or propyl.

In one embodiment for compounds of Formula (I), R¹ is hydroxyalkyl.

In one embodiment for compounds of Formula (I), R¹ is —Y-heterocyclyl. In certain embodiments, Y is a bond and the heterocyclyl is azetidinyl, THFyl or morpholinyl.

In one embodiment for compounds of Formula (I), R¹ is —Y-aryl wherein the aryl is optionally substituted with one or more R².

In certain embodiments, Y is a bond and the aryl is phenyl optionally substituted with one or two R². In one embodiment, the one or two R² groups are each independently C1-C3 alkyl, cyano or halogen.

In one embodiment for compounds of Formula (I), R¹ is —Y-cycloalkyl. In one embodiment, Y is a bond and the cycloalkyl is cyclopentyl.

In one embodiment for compounds of Formula (I), R¹ is —Y-heteroaryl optionally substituted with one or more R². In certain embodiment, the heteroaryl is, pyrazolyl, imidazolyl, thiazolyl, isothiazolyl, oxazolyl, triazolyl, oxidazolyl, pyridyl, pyridiazinyl, pyrimidinyl, quinolinyl, isoquinolinyl, phthalazinyl, pyrazolopyridinyl, 1H-pyrrolopyridyl, pyrazolopyrimidinyl, imidazopyridyl, tetrahydropyrazolopyrazinyl, 2H-4λ/⁴-imidazopyrimidinyl, 2H-4λ/⁴-imidazopyridazinyl, oxazolopyridyl or 5,6-dihydro-8H-imidazooxazinyl, each optionally substituted with one or more R². In one embodiment, Y is a bond.

In one embodiment, R¹ is —Y-heteroaryl, Y is a bond and the heteroaryl is azetidinyl and R² is —(NR⁴)₂.

In one embodiment, R¹ is heteroaryl, Y is a bond and the heteroaryl is tetrahydropyrazolopyrazinyl, optionally substituted with one or more R². In one embodiment, the tetrahydropyrazolopyrazinyl is 4,5,6,7-tetrahydropyrazolo[1,5-a]pyrazin-3-yl optionally substituted with one or more R². In one embodiment, the tetrahydropyrazolopyrazinyl is substituted with one R². In one embodiment, R² is —X—C1-C5 alkyl, arC1-C3alkyl, —Z—C1-C5 alkyl, —Z-cycloalkyl or —X-aryl. In one embodiment, R² is —Z-cycloalkyl, wherein Z is a bond and the cycloalkyl is cyclopropyl. In one embodiment, R² is —Z-cycloalkyl, wherein Z is —C(O)— and the cycloalkyl is cyclopropyl, cyclobutyl, cyclopentyl, cyclohexyl or bicyclo[1.1.1]pentyl.

In one embodiment wherein R¹ is Y-heteroaryl, Y is a bond and the heteroaryl is pyrazolopyridinyl optionally substituted with one or more R². In one embodiment, the pyrazolylpyridinyl is substituted with one R², wherein the one R² is alkoxy or —X-aryl. In one embodiment, the alkoxy is methoxy or isopropyloxy. In certain embodiments, the —X-aryl, the X is O and the aryl is phenyl.

In one embodiment, Y is a bond and the R¹ heteroaryl is pyridyl, optionally substituted with one or two R². In certain embodiments, the pyridyl is substituted with one R², wherein R² is hydroxy, halogen, cyano, cyanomethyl, —(NR⁴)₂, hydroxyalkyl, alkoxy, —SO₂C1-C3alkyl, arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵.

In one embodiment, R¹ is —Y-heteroaryl, Y is a bond and the heteroaryl is pyridyl and R² is —X—C1-C5 alkyl, X is a bond and the C1-C5 alkyl is methyl, ethyl propyl, isopropyl, butyl, isobutyl, pentyl or isopentyl.

In one embodiment, R¹ is —Y-heteroaryl, Y is a bond and the heteroaryl is pyridyl and R² is —X-haloalkyl, X is a bond and the haloalkyl is difluoromethyl or trifluoromethyl. In another embodiment, R² is —X-haloalkyl, wherein X is O, and wherein the haloalkyl is difluoromethyl or trifluoromethyl.

In one embodiment, R¹ is —Y-heteroaryl, Y is a bond and the heteroaryl is pyridyl and R² is —X-L-cycloalkyl, wherein X is a bond, L is a bond and the cycloalkyl is cyclopropyl or cyclohexyl. In another embodiment, R² is —X-L-cycloalkyl, wherein X is a bond, L is methylene and the cycloalkyl is cyclopropyl. In one embodiment, R² is —X-L-cycloalkyl, wherein X is O, L is methylene and the cycloalkyl is cyclopropyl.

In one embodiment, R¹ is —Y-heteroaryl, Y is a bond and the heteroaryl is pyridyl and R² is C2-C4 alkynyl, wherein the alkynyl is ethynyl or prop-2-ynyl.

In another embodiment, R¹ is —Y-heteroaryl, Y is a bond and the heteroaryl is pyridyl and R² is —SO₂C1-C3 alkyl, wherein the C1-C3 alkyl is methyl.

In one embodiment, R¹ is —Y-heteroaryl, Y is a bond and the heteroaryl is pyridyl and R² is heterocyclyl, wherein the heterocyclyl is morpholinyl or tertrahydropyranyl.

In other embodiments, R¹ is —Y-heteroaryl, Y is a bond and the heteroaryl is pyridyl and R² is —X-heteroaryl, wherein the heteroaryl is optionally substituted with one or more R⁵. In one embodiment, X is a bond, the heteroaryl is pyrazolyl substituted with one R⁵, wherein R⁵ is C1-C3 alkyl. In one embodiment, X is a bond, the heteroaryl is pyridyl or pyrimidinyl, each optionally substituted with one R⁵.

In one embodiment, R¹ is —Y-heteroaryl, Y is a bond and the heteroaryl is pyridyl and R² is arC1-C3alkyl, wherein the arC1-C3alkyl is benzyl.

In one embodiment wherein R¹ is —Y-heteroaryl, Y is a bond and the heteroaryl is pyridyl and R² is —X-heteroaryl, wherein the X is O, and the heteroaryl is quinolinyl optionally substituted with one or more R⁵. In another embodiment, the X is —NR⁴—, and the heteroaryl is quinolinyl optionally substituted with one or more R⁵.

In one embodiment, R¹ is —Y-heteroaryl, Y is a bond and the heteroaryl is pyridyl and R² is —X-aryl, wherein X is O and the aryl is phenyl optionally substituted with one, two or three R⁵. In one embodiment, each of the one, two or three R⁵ groups is independently selected from the group consisting of cyano, halogen, C1-C3 alkyl and alkoxy. In one embodiment, X is S and the aryl is phenyl optionally substituted with one R⁵, wherein R⁵ is halogen or C1-C3 alkyl. In one embodiment, X is O and the aryl is phenyl optionally substituted with two R⁵ groups, wherein each R⁵ group is independently cyano. In one embodiment, X is —NR⁴— and the aryl is phenyl optionally substituted with two R⁵ groups, wherein each R⁵ group is independently alkoxy. In certain embodiments, each alkoxy is methoxy.

In one embodiment, R¹ is —Y-heteroaryl, Y is a bond and the heteroaryl is pyridyl and R² is halogen, wherein the halogen is chlorine or fluorine. In one embodiment, R¹ is —Y-heteroaryl, Y is a bond and the heteroaryl is pyridyl and R² is —X-L-cycloalkyl, heterocyclyl or —X-aryl, wherein the aryl is optionally substituted with one or more R⁵. In one embodiment, R² is-X-L-cycloalkyl, wherein X and L are each a bond and the cycloalkyl is cyclohexyl. In one embodiment, R² is heterocyclyl, wherein the heterocyclyl is tetrahydropyranyl. In one embodiment, R² is —X-aryl, wherein the aryl is phenyl substituted with two R⁵, wherein each R⁵ is cyano.

In certain embodiments, R¹ is —Y-heteroaryl, Y is a bond and the heteroaryl is pyridyl substituted with two R². In one embodiment, each R² is independently —X—C1-C5 alkyl or one R² is halogen or cycloalkyl and the second R² is —X—C1-C5 alkyl, wherein X is a bond.

In one embodiment for compounds of Formula (I), R¹ is —Y-heteroaryl, Y is a bond and the heteroaryl is pyrimidinyl, optionally substituted with one or two R². In one embodiment, the pyrimidinyl is substituted with one R², wherein R² is —X—C1-C5 alkyl or —X-haloalkyl. In one embodiment, each X is a bond.

In one embodiment for compounds of Formula (I), R¹ is —Y-heteroaryl, Y is a bond and the heteroaryl is quinolinyl, optionally substituted with one or two R². In certain embodiments, the one R² group is cyano. In certain embodiments, one R² group is cyano and the second R² is halogen or —X—C1 C5 alkyl.

In one embodiment for compounds of Formula (I), R¹ is —Y-heteroaryl, Y is a bond and the heteroaryl is isothiazolyl, optionally substituted with one or two R². In one embodiment, R² is —X-aryl optionally substituted with one R⁵, wherein the aryl is naphthyl substituted with one R⁵, wherein R⁵ is cyano.

In one embodiment for compounds of Formula (I), R¹ is —Y-heteroaryl, Y is a bond and the heteroaryl is pyrazolyl, optionally substituted with one, two or three R² groups.

In certain embodiments, the pyrazolyl is substituted with one R², wherein R² is cyano, —X—C1-C5 alkyl, hydroxyalkyl, arC1-C3alkyl or —X-aryl, wherein the aryl is optionally substituted with one or more R⁵. In one embodiment, R² is —X—C1-C5 alkyl, wherein X is a bond and the C1-C5 alkyl is methyl, ethyl, propyl, isopropyl, butyl, isobutyl, pentyl or isopentyl.

In other embodiments, the pyrazolyl is substituted with two R² groups, wherein the two R² groups are independently (1) —X—C1-C5 alkyl, (2) —X—C1-C5 alkyl and halogen, (3) —X—C1-C5 alkyl and alkoxy, (4) —X—C1-C5 alkyl and —N(R⁴)₂, -(5) X—C1-C5 alkyl and —X-haloalkyl, (6) —X—C1-C5 alkyl and arC1-C3alkyl, (7) —X-C1-C5 alkyl and —X-L-cyclolalkyl, -(8) —X-C1-C5 alkyl and -heterocyclyl, (9) —X—C1-C5 alkyl and —X-aryl optionally substituted with one or more R⁵, (10) —X-C1-C5 alkyl and —X-heteroaryl optionally substituted with one or more R⁵, (11) —X—C1-C5 alkyl and cyanomethyl, (12) —X—C1-C5 alkyl and cyano, (13) cyano and halogen, wherein the halogen is chlorine or fluorine, (14) cyano and —X-L-cycloalkyl, (15) independently halogen, (16) cyano and alkoxy, wherein each X is a bond, (17) cyano and —X-aryl, (18) cyano and —X-heteroaryl, (19) cyano and heterocyclyl (20) halogen and —X-arC1-C3alkyl or X-arC1-C3alkyl substituted with cyano, and (21) halogen and —X-aryl.

In one embodiment wherein R¹ is pyrazolyl, the pyrazolyl is substituted with two R², wherein one R² is —X—C1-C5 alkyl and the second R² is —X-aryl optionally substituted with one or more R⁵. In one embodiment, each X is a bond and the aryl is phenyl substituted with two R⁵, wherein (1) each R⁵ is independently —X—C1-C5 alkyl, wherein X is a bond; (2) one R⁵ is cyano and one R⁵ is —X-C1-C5 alkyl, wherein X is a bond; (3) one R⁵ is cyano and one R⁵ is —X-L-cycloalkyl, wherein X is a bond and L is a bond, methylene or ethylene: (4) one R⁵ is cyano and one R⁵ is halogen; (5) one R⁵ is cyano and one R⁵ is alkoxy; (6) each R⁵ is independently cyano or (7) each R⁵ is independently halogen.

In one embodiment wherein R¹ is pyrazolyl, the pyrazolyl is substituted with two R², wherein one R² is —X—C1-C5 alkyl and the second R² is —X-aryl optionally substituted with one or more R⁵. In one embodiment, the X is a bond and the aryl is naphthyl substituted with one R⁵, wherein R⁵ is cyano or halogen. In one embodiment, the naphthyl is substituted with two R⁵ groups, wherein one R⁵ is cyano and the second R⁵ is halogen, alkoxy or cyano. In one embodiment, the naphthyl is substituted with three R⁵ groups, wherein one R⁵ is cyano and the second R⁵ is X-haloalkyl and the third R⁵ is —X-L-cycloalkyl.

In one embodiment wherein R¹ is pyrazolyl, the pyrazolyl is substituted with two R², wherein one R² is —X-C1-C5 alkyl and the second R² is —X-aryl optionally substituted with one or more R⁵. In one embodiment, the X is a bond and the aryl is phenyl substituted with three R⁵, wherein (1) each R⁵ is independently —X—C1-C5 alkyl, wherein each X is a bond; (2) one R⁵ is cyano and two R⁵ are —X—C1-C5 alkyl, wherein each X is a bond; (3) one R⁵ is cyano, one R⁵ is halogen, and one R⁵ is —X—C1-C5 alkyl, wherein X is a bond; (4) one R⁵ is cyano and two R⁵ are alkoxy, (5) one R⁵ is cyano and two R⁵ are halogens (6) one R⁵ is cyano, one R⁵ is halogen and one R⁵ is alkoxy, (7) or one R⁵ is cyano, one R⁵ is halogen, and one R⁵ is —X-L-cycloalkyl.

In one embodiment wherein R¹ is pyrazolyl, the pyrazolyl is substituted with two R², wherein one R² is —X—C1-C5 alkyl and the second R² is —X-heteroaryl optionally substituted with one or more R⁵. In one embodiment, each X is a bond and the heteroaryl is quinolinyl, pyrazolyl, chromanyl, indolizinyl, dihydrobenzylfuranyl or imidzaopyridinyl, each optionally substituted with one or more R⁵.

In one embodiment, the pyrazolyl is substituted with three R², wherein each R² is independently —X—C1-C5 alkyl and each X is a bond.

In one embodiment wherein R¹ is pyrazolyl, the pyrazolyl is substituted with three R², wherein (1) one R² is cyano and two R² are halogen; (2) one R² is cyano, one R² is halogen and one R² is alkoxy. In other embodiments, one R² is alkoxy, and two R² are independently halogen

In one embodiment for compounds of Formula (I), R¹ is —Y-heteroaryl, Y is a bond and the heteroaryl is imidazolyl, 1H-pyrrolopyridyl, tetrahydropyrazolopyrazinyl, 2H-4λ/⁴-imidazopyrimidinyl, 2H-4λ/⁴-imidazopyridazinyl, or oxazolopyridyl, each substituted with one R² group, wherein R² is —X—C1-C5 alkyl, wherein X is a bond. In one embodiment, the heteroaryl is 1H-pyrrolopyridyl substituted with one R², wherein R² is cyano or —X-aryl. In certain embodiments, the X of the —X-aryl is a bond and the aryl is phenyl. In one embodiment, the heteroaryl is imidazolyl substituted with one R², wherein R² is hydroxyalkyl or —X-aryl.

In one embodiment for compounds of Formula (I), R¹ is —Y-heteroaryl, Y is a bond and the heteroaryl is imidazopyridyl substituted with one R² group, wherein R² is cyano, alkoxy, halogen or —X—C1-C5 alkyl. In other embodiments, the heteroaryl is imidazopyridyl substituted with two R² groups, wherein one R² is halogen and the second R² group is —X-C1-C5 alkyl or halogen.

In one embodiment for compounds of Formula (I), R¹ is —Y-aryl, Y is —NR⁴— and the aryl is phenyl optionally substituted with one or more R⁵.

In one embodiment, R¹ is —Y-arC1-C3alkyl. In one embodiment, Y is —NR⁴— and the arC1-C3alkyl is benzyl.

In one embodiment, R^{3a} and R^{3b} are each hydrogen. In another embodiment, R^{3a} and R^{3b} are each deuterium. In certain embodiments, one of R^{3a} and R^{3b} is hydrogen and the other is deuterium. In one embodiment, R^{3a} and R^{3b} taken together are oxo.

In one embodiment, each R⁴ is hydrogen. In one embodiment, each R⁴ is independently C1-C3 alkyl. In one embodiment, one R⁴ is hydrogen and the other R⁴ is C1-C3 alkyl.

In one embodiment, the cycloalkyl, aryl or heteroaryl rings are optionally substituted with one or more R⁵, wherein R is cyano, oxo, halogen, C1-C3 alkyl, hydroxyalkyl, alkoxy, —X-haloalkyl, —Z-cycloalkyl, —X-arC1-C3alkyl, —X-L-cycloalkyl or —X-aryl.

In one embodiment, R⁶ is hydrogen. In one embodiment, R⁶ is halogen. In certain embodiments, the halogen is chlorine or fluorine. In one embodiment, R⁶ is C1-C3 alkyl. In certain embodiments, the C1-C3 alkyl is methyl or ethyl. In one embodiment, R⁶ is alkoxy. In certain embodiments, the alkoxy is methoxy. In one embodiment, R⁶ is haloalkyl. In certain embodiments, the haloalkyl is trifluoromethyl.

In one aspect of the invention, compounds are provided that are represented by Formula (I-A):

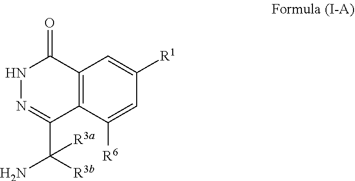

or a pharmaceutically acceptable salt thereof, wherein R¹, R², R^{3a}, R^{3b}, R⁴, R⁵, R⁶, Y, X, Z, and L are as each defined for Formula I.

In one aspect of the invention, compounds are provided that are represented by Formula (I-B):

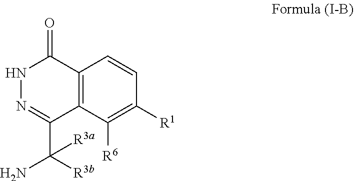

or a pharmaceutically acceptable salt thereof, wherein R¹, R², R^{3a}, R^{3b}, R⁴, R⁵, R⁶, Y, X, Z, and L are as each defined for Formula I.

In one aspect of the invention, compounds are provided that are represented by Formula

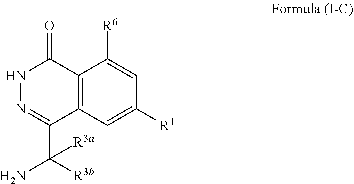

or a pharmaceutically acceptable salt thereof, wherein R¹, R², R^{3a}, R^{3b}, R⁴, R⁵, R⁶, Y, X, Z and L are as each defined for Formula I.

In another aspect of the invention, compounds are provided that are represented by Formula (I-D):

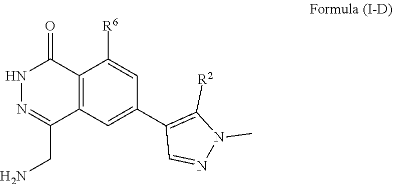

or a pharmaceutically acceptable salt thereof:
wherein:
each Y is independently a bond or —NR⁴—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵, or;
each X is independently a bond, O, S, —NR⁴— or —NR⁴C(O)—;
each Z is independently a bond, —SO—, —SO₂—, —CH(OH)— or —C(O)—;
each L is independently a bond or C1-C3 alkylene;
each R⁴ is independently hydrogen or C1-C3 alkyl;
each R⁵ is independently cyano, oxo, halogen, C1-C3 alkyl, hydroxyalkyl, alkoxy, —X— haloalkyl, —Z-cycloalkyl, —X-arC1-C3alkyl, X-arC1-C3alkyl substituted with cyano, —X-L-cycloalkyl, —X-L-heteroaryl optionally substituted with one or more C1-C3alkyl or oxo, or —X-aryl; and
R⁶ is hydrogen, halogen, C1-C3 alkyl, haloalkyl or alkoxy.

The compounds of Formula (I), Formula (I-A), Formula (I-B) and Formula (I-C) may be formulated into pharmaceutical compositions.

One aspect of the invention also include those wherein there is provided a compound of Formula IIA, IIA-1, IIB, IIB-1, IC or IIC-1 (Embodiment 1):

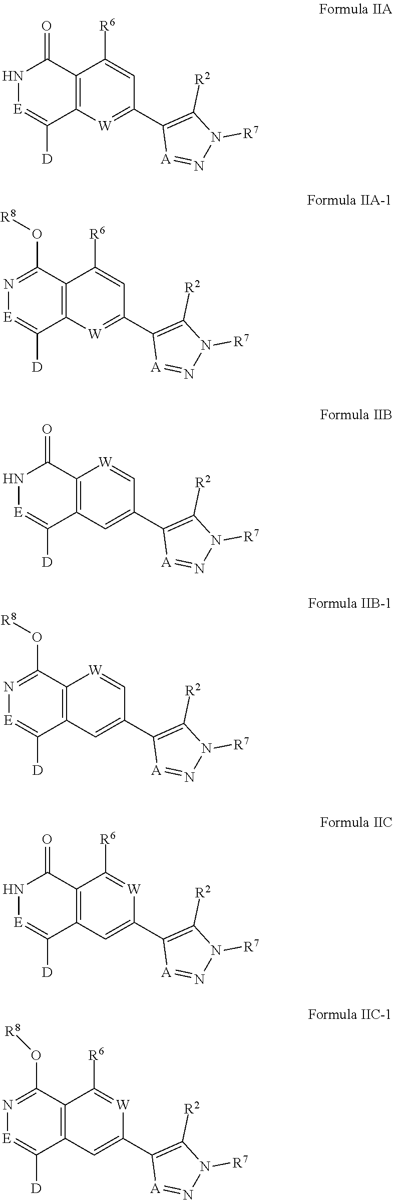

or a pharmaceutically acceptable salt thereof, wherein:
A is CR⁹ or N;

D is (C(R⁹)₂)₁₋₂—NHR¹¹,

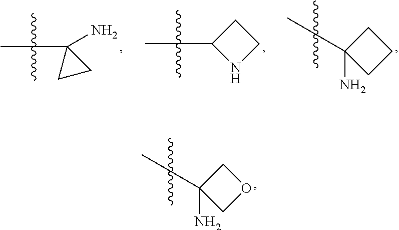 [SMILES: *C1(N)CC1 ; *C1CCN1 ; *C1(N)CCC1 ; *C1(N)COC1]

or D is

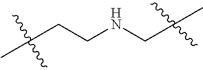 [SMILES: *CCNC*]

where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵, or;
each R⁴ is independently hydrogen or C1-C3 alkyl;
each R⁵ is independently cyano, oxo, halogen, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R³ is C1-C3 alkyl;
each R⁹ is independently H or C1-C3 alkyl, halogen or haloalkyl; and
R¹¹ represents hydrogen or —C(O)R², where R² is hydrogen or C1-C3 alkyl.

Embodiments of the invention include those wherein there is provided a compound of Formula IIA (Embodiment 2):

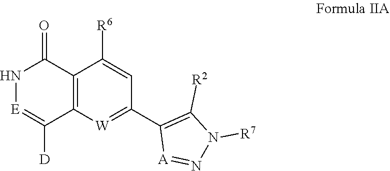

or a pharmaceutically acceptable salt thereof, wherein:
A is CR⁹ or N;

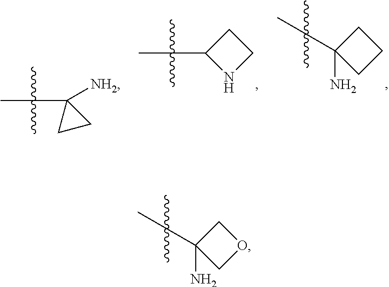 [SMILES: *C1(N)CC1 ; *C1CCN1 ; *C1(N)CCC1 ; *C1(N)COC1]

D is (C(R⁹)₂)₁₋₂—NH₂,

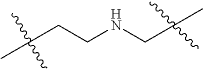 [SMILES: *CCNC*]

where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵, or;
each R⁴ is independently hydrogen or C1-C3 alkyl;
each R⁵ is independently cyano, oxo, halogen, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl; and
each R⁹ is independently H or C1-C3 alkyl, halogen or haloalkyl.

Embodiments of the invention also include those wherein there is provided a compound of Formula IIB (Embodiment 3):

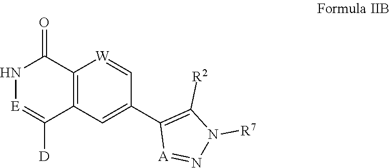

or a pharmaceutically acceptable salt thereof, wherein:
A is CR⁹ or N;

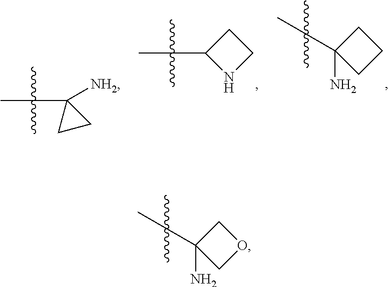 [SMILES: *C1(N)CC1 ; *C1CCN1 ; *C1(N)CCC1 ; *C1(N)COC1]

D is (C(R⁹)₂)₁₋₂—NH₂,

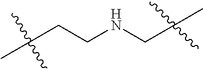 [SMILES: *CCNC*]

where the methylene is bonded to E where E is C:
E is C, CR⁹ or N;
each L is independently a bond or C1-C3 alkylene;
W is CR⁹ or N:
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z-C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵, or;
each R⁴ is independently hydrogen or C1-C3 alkyl;
each R⁵ is independently cyano, oxo, halogen, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl; and
each R⁹ is independently H or C1-C3 alkyl, halogen or haloalkyl.

Embodiments of the invention further include those wherein there is provided a compound of Formula IIC or IIC-1 (Embodiment 4):

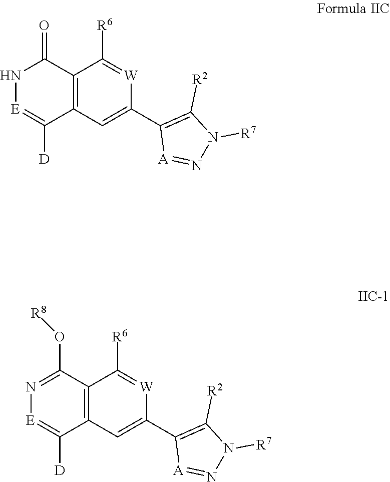

or a pharmaceutically acceptable salt thereof, wherein:
A is CR⁹ or N;

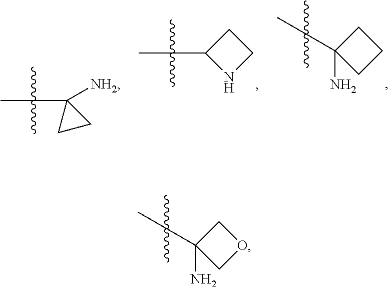 [SMILES: *C1(N)CC1 ; *C1CCN1 ; *C1(N)CCC1 ; *C1(N)COC1]

D is (C(R⁹)₂)₁₋₂—NH₂,

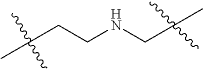 [SMILES: *CCNC*]

where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z-C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵, or;
each R⁴ is independently hydrogen or C1-C3 alkyl;
each R⁵ is independently cyano, oxo, halogen, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl; and
each R⁹ is independently H or C1-C3 alkyl, halogen or haloalkyl.

In a particular embodiment (embodiment 4λ) of Formulas IIA, IIB, IIC and IIC-1, D is (C(R⁹)₂)₁₋₂—NHR¹¹,

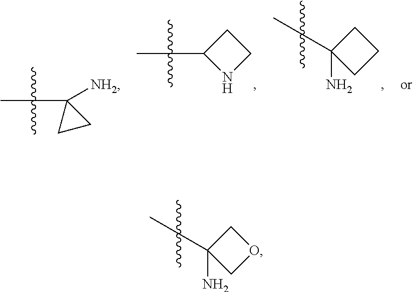 [SMILES: *C1(N)CC1 ; *C1CCN1 ; *C1(N)CCC1 ; *C1(N)COC1]

Embodiment 5 provides the compound or salt of any of embodiments 1-4 and 4λ, wherein W is CR⁹.

Embodiment 6 provides the compound or salt of any of embodiments 1-4 and 4λ, wherein A is CR⁹.

Embodiment 7 provides the compound or salt of any of embodiments 1-4 and 4λ, wherein E is N.

Embodiment 8 provides the compound or salt of any of embodiments 1-7, wherein W is CR⁹, A is CR⁹ and E is N.

Embodiment 9 provides the compound or salt of any of embodiments 1-8, wherein R² is selected from: benzothiophene, naphthalene, quinoline, chromane, isochromane, dihydrobenzodioxine, indolazine, tetrahydroindolazine, dihydroisobenzofuran, benzene, isoquinolinone, benzodioxone, thienopyridine, tetrahydroindolone, indolizine, dihydroindolizinone, imadazopyridinone, thienopyrimidine, thiophene, pyrrolopyrimidinone, thiazolopyridinone, dihydropyrrolizine, isoindalone and tetrahydroisoquinoline.

Embodiment 10 provides the compound or salt of any of embodiments 1-8, wherein each R⁵ is independently cyano, oxo, halogen, C1-C3 alkyl, hydroxy, hydroxyalkyl, alkoxy-C1-C3alkyl, —X-L-heterocyclyl optionally substituted with one or more C1-C3alkyl or oxo, —X-L-cycloalkyl optionally substituted with C1-C3 alkyl or oxo.

Embodiment 11 provides the compound or salt of any of embodiments 1-8, wherein R⁶ is selected from hydrogen, hydroxy, chlorine, —NHC(O)CH₃, —C(O)CF₂H, —NH₂, —CF₂, —CH₃, —O—CH₂CH₃, —CH₂—CH₂—O—CH₃, oxetane and THF.

Embodiment 12 provides the compound or salt of any of embodiments 1-11, where one of L, X and Z is a bond.

Embodiment 13 provides the compound or salt of embodiment 12, wherein all of L, X and Z are bonds.

One aspect of the disclosure provides a compound of the formula (IIA) (Embodiment 14):

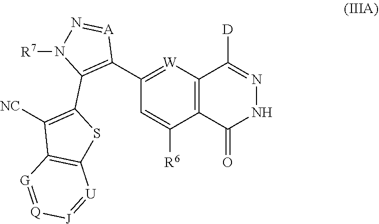

or a pharmaceutically acceptable salt thereof, wherein
A is CR⁹ or N;
D is —CH₂—NH₂,

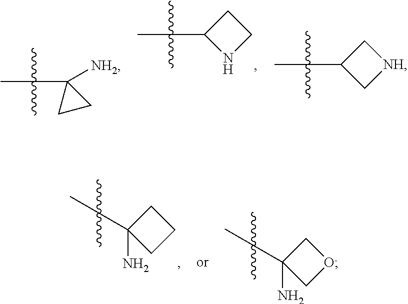 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶,
   where each R⁹ is independently H or C₁-C₃ alkyl, R¹⁵ is hydrogen or methyl, and R¹⁶ is C₁-C₃ alkyl; and
R⁷ is C₁-C₃ alkyl or C₁-C₃ haloalkyl,
provided that the compound is not 2-(4-(4-(aminomethyl)-1-oxo-1,2-dihydrophthalazin-6-yl)-1-methyl-1H-pyrazol-5-yl)benzo[b]thiophene-3-carbonitrile or 2-(4-(4-(aminomethyl)-1-oxo-1,2-dihydrophthalazin-6-yl)-1-methyl-1H-pyrazol-5-yl)thieno[2,3-b]pyridine-3-carbonitrile.

Embodiment 15 provides the compound according to embodiment 14, wherein A is CH.

Embodiment 16 provides the compound according to embodiment 14 or 15, wherein W is N.

Embodiment 17 provides the compound according to embodiment 14 or 15, wherein W is CH.

Embodiment 18 provides the compound according to any of embodiments 14-17, wherein D is —CH₂—NH₂.

Embodiment 19 provides a compound according to embodiment 14, which is of the formula:

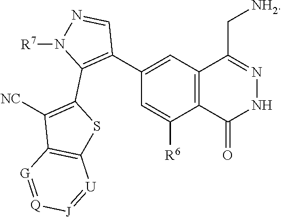

Embodiment 20 provides the compound according to any of embodiments 14-19, wherein R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 21 provides the compound according to any of embodiments 14-19, wherein R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 22 provides the compound according to any of embodiments 14-19, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 23 provides the compound according to any of embodiments 14-19, wherein R⁶ is halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹¹.

Embodiment 24 provides the compound according to any of embodiments 14-19, wherein R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 25 provides the compound according to any of embodiments 14-19, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 26 provides the compound according to any of embodiments 23-25, wherein each G, Q, J and U is independently C(H).

Embodiment 27 provides the compound according to any of embodiments 23-25, wherein G, Q, J and U are independently selected from C(H) and C(R⁵).

Embodiment 28 provides the compound according to any of embodiments 23-25, wherein G, Q, J and U are independently selected from C(H) and N.

Embodiment 29 provides the compound according to any of embodiments 14-19, wherein
R⁶ is hydrogen;
at least one of G, Q, J, and U is C(R⁵), and the remaining G, Q, J, and U are independently selected from C(H), C(R⁵) and N, wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 30 provides the compound according to embodiment 29, wherein one or two of G, Q, J and U is N.

Embodiment 31 provides the compound according to any of embodiments 14-19, wherein
R⁶ is hydrogen;
at least one of G, Q, J, and U is C(R⁵), and the remaining G, Q, J, and U are independently selected from C(H) and C(R⁵), wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 32 provides the compound according to embodiment 31, wherein at least one of G, Q, J, and U is C(R⁵), and the remaining G, Q, J, and U are independently C(H); for example only one of G, Q, J, and U is C(R⁵).

Embodiment 33 provides the compound according to embodiment 31, wherein two of G, Q, J, and U is C(R⁵), and the remaining G, Q, J, and U are independently C(H).

Embodiment 34 provides the compound according to embodiment 31, wherein three of G, Q, J, and U is C(R⁵), and the remaining G, Q, J, and U is C(H).

Embodiment 35 provides a compound according to any of embodiments 14-19, wherein G, Q, J, and U together with the thiophene to which they are attached form:

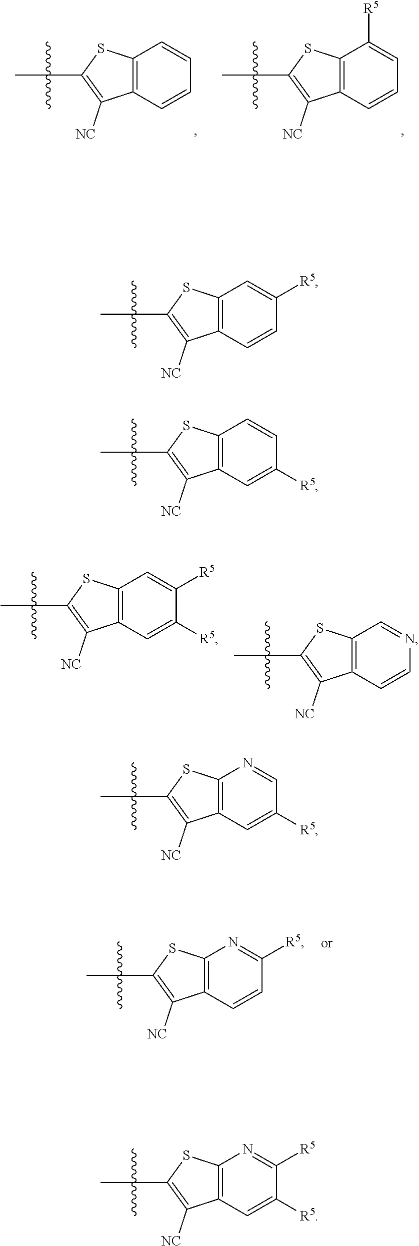

Embodiment 36 provides the compound according to embodiment 35, wherein G, Q, J, and U together with the thiophene ring to which they are attached form a benzo[b]thiophene.

Embodiment 37 provides the compound according to any one of embodiments 14-36, wherein R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 38 provides the compound according to any one of embodiments 14-36, wherein R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

Embodiment 39 provides the compound according to any one of embodiments 14-36, wherein R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

Embodiment 40 provides the compound according to any one of embodiments 14-39, wherein R⁷ is methyl.

Embodiment 41 provides the compound according to any one of embodiments 14-39, wherein R⁷ is ethyl.

Embodiment 42 provides the compound according to any one of embodiments 14-39, wherein R⁷ is propyl (e.g., isopropyl).

Embodiment 43 provides the compound according to any one of embodiments 14-39, wherein R⁷ is difluoromethyl or trifluoromethyl.

Embodiment 44 provides a compound according to embodiment 14, which is of the formula:

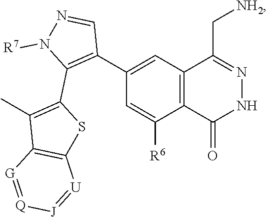

wherein
G, Q, J, and U together with the thiophene to which they are attached form:

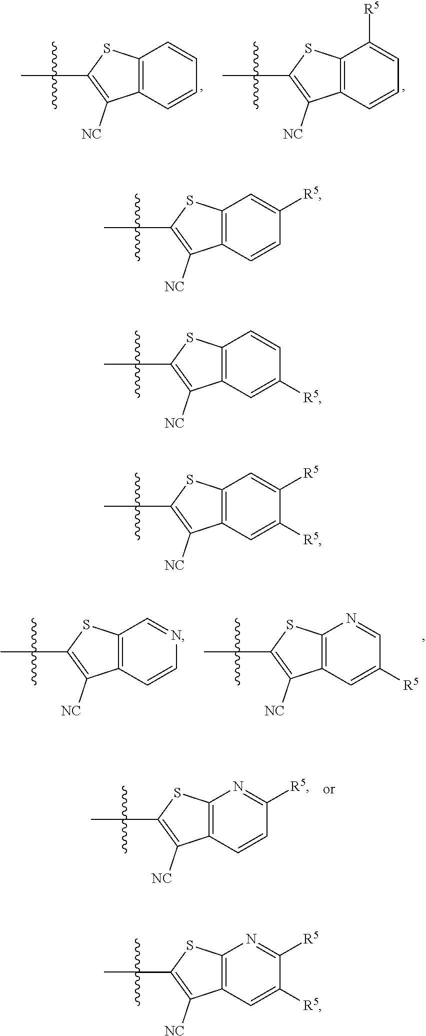

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 45 provides the compound according to embodiment 14, which is of the formula:

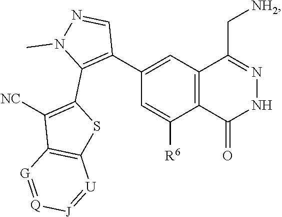

wherein
G, Q, J, and U together with the thiophene to which they are attached form:

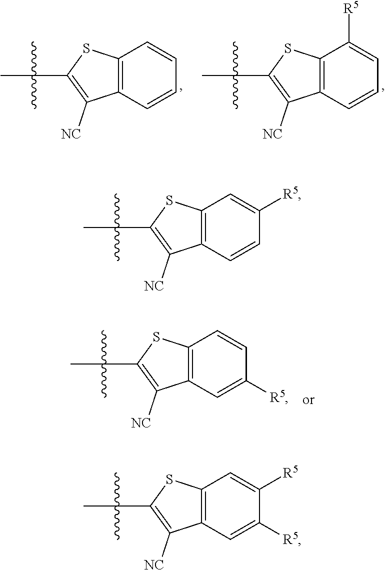

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 46 provides a compound according to embodiment 14, which is of the formula:

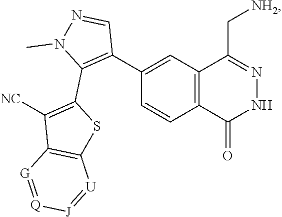

wherein
G, Q, J, and U together with the thiophene to which they are attached form:

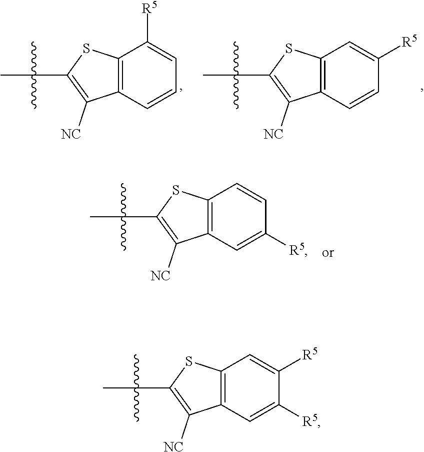

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

Embodiment 47 provides a compound of the formula (IIIB):

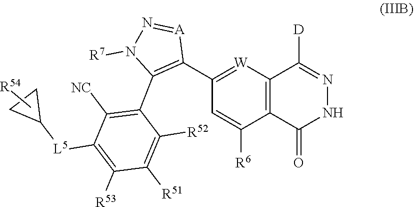

or a pharmaceutically acceptable salt thereof, wherein
A is CR⁹ or N;
D is —CH₂—NH₂,

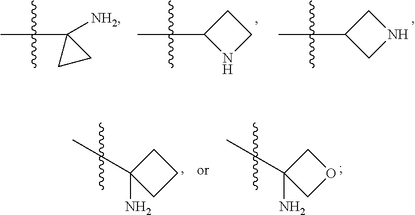 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R⁵¹ is hydrogen, fluoro, chloro, or methyl, or R⁵¹ and R⁵² together with atoms to which they are attached form a C₄-C₆ heterocycloalkyl (e.g, hydrofuranyl);
R⁵² is fluoro, chloro, or methyl, or R² and R³ together with atoms to which they are attached form a phenyl;
R⁵³ is hydrogen, fluoro, chloro, or methyl;
R⁵⁴ is hydrogen, halogen, C₁-C₃ alkyl, or C₁-C₃ alkoxy;
L⁵ is —O— or —CH₂—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl;
R⁷ is C₁-C₃ alkyl or C₁-C₃ haloalkyl,
provided that the compound is not 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, 2-(4-(4-(aminomethyl)-1-oxo-1,2-dihydrophthalazin-6-yl)-1-methyl-1H-pyrazol-5-yl)-6-cyclopropoxy-3-fluoro-4-methylbenzonitrile, 2-(4-(4-(aminomethyl)-1-oxo-1,2-dihydrophthalazin-6-yl)-1-methyl-1H-pyrazol-5-yl)-3-chloro-6-cyclopropoxybenzonitrile, 2-(4-(4-(aminomethyl)-8-chloro-1-oxo-1,2-dihydrophthalazin-6-yl)-1-methyl-1H-pyrazol-5-yl)-6-cyclopropoxy-3-fluoro-4-methylbenzonitrile, or 2-(4-(4-(aminomethyl)-1-oxo-1,2-dihydrophthalazin-6-yl)-1-methyl-1H-pyrazol-5-yl)-6-cyclopropoxy-3-fluorobenzonitrile.

Embodiment 48 provides the compound according to embodiment 47, wherein A is CH.

Embodiment 49 provides the compound according to embodiment 47 or 48, wherein W is N.

Embodiment 50 provides the compound according to embodiment 47 or 48, wherein W is CH.

Embodiment 51 provides the compound according to any of embodiments 47-50, wherein D is —CH₂—NH₂.

Embodiment 52 provides the compound according to any of embodiments 47-51, wherein R⁵⁴ is hydrogen or methyl.

Embodiment 53 provides the compound according to any of embodiments 47-51, wherein R⁵⁴ is hydrogen.

Embodiment 54 provides the compound according to any of embodiments 47-51, wherein R⁵⁴ is methyl.

Embodiment 55 provides a compound according to embodiment 47, which is of the formula:

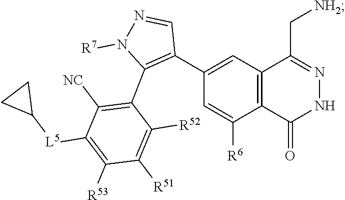

such as e.g.,

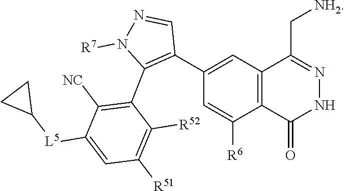

Embodiment 56 provides the compound according to any of embodiments 47-55, wherein L⁵ is —CH₂—.

Embodiment 57 provides the compound according to any of embodiments 47-55, wherein L is —O—.

Embodiment 58 provides the compound according to any of embodiments 47-57, wherein R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 59 provides the compound according to any of embodiments 47-57, wherein R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

Embodiment 60 provides the compound according to any of embodiments 47-57, wherein R⁶ is hydrogen, chloro, fluoro, methyl, ethyl, methoxy, or ethoxy.

Embodiment 61 provides the compound according to any of embodiments 47-57, wherein R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 62 provides the compound according to any of embodiments 47-57, wherein R⁶ is halogen, C₁-C₆ alkyl, or C₁-C₆ alkoxy; for example, R⁶ is halogen, C₁-C₃ alkyl, or C₁-C₃ alkoxy.

Embodiment 63 provides the compound according to any of embodiments 47-57, wherein R⁶ is chloro, fluoro, methyl, ethyl, methoxy, or ethoxy.

Embodiment 64 provides the compound according to any one of embodiments 47-63, wherein R⁷ is methyl.

Embodiment 65 provides the compound according to any one of embodiments 47-63, wherein R⁷ is ethyl.

Embodiment 66 provides the compound according to any one of embodiments 47-63, wherein R⁷ is propyl (e.g., isopropyl).

Embodiment 67 provides the compound according to any one of embodiments 47-63, wherein R⁷ is difluoromethyl or trifluoromethyl.

Embodiment 68 provides the compound according to any of embodiments 47-67, wherein R⁵³ is hydrogen or methoxy; or wherein R⁵³ is hydrogen.

Embodiment 69 provides a compound according to embodiment 47, which is of the formula:

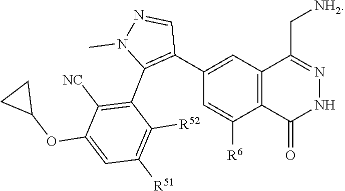

Embodiment 70 provides the compound according to any one of embodiments 47-69, wherein R⁵² is fluoro, and R⁵¹ is hydrogen, fluoro, chloro, or methyl.

Embodiment 71 provides the compound according to any one of embodiments 47-69, wherein R⁵² is fluoro, and R⁵¹ is chloro.

Embodiment 72 provides the compound according to any one of embodiments 47-69, wherein R⁵² is fluoro, and R⁵¹ is methyl or hydrogen (for example, R⁵² is fluoro and R⁵¹ is methyl; or R⁵² is fluoro and R⁵¹ is hydrogen).

Embodiment 73 provides the compound according to any one of embodiments 47-69, wherein R⁵¹ and R⁵² to ether with atoms to which they are attached form a hydrofuranyl e.g.,

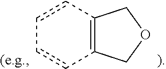

One aspect of the disclosure provides a compound of the formula (IIIC) (Embodiment 74):

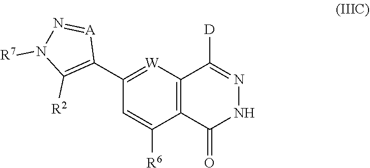

or a pharmaceutically acceptable salt thereof, wherein
A is CR⁹ or N;
is —CH₂—NH₂,

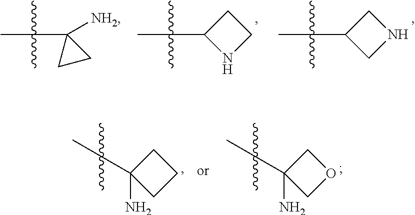 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

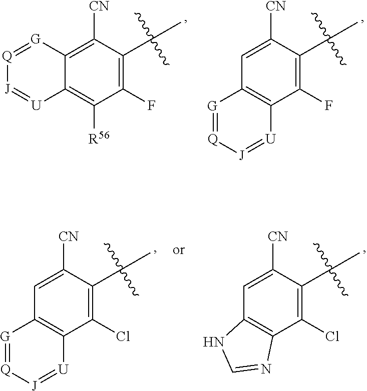

where R⁵⁶ is hydrogen, fluoro, chloro, or methyl,
G, Q, J and U are independently selected from C(H), C(R⁵), and N, provided only one or two of G, Q, J, and U can be N, and provided that when R¹ is hydrogen at least one of G, Q, J and U is C(R⁵) or N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

One aspect of the disclosure provides a compound of the formula (IIIC) (Embodiment 75):

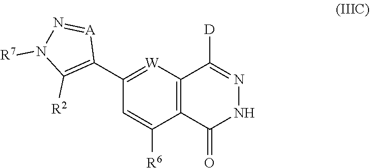

or a pharmaceutically acceptable salt thereof, wherein
A is CR or N,
D is —CH₂—NH₂,

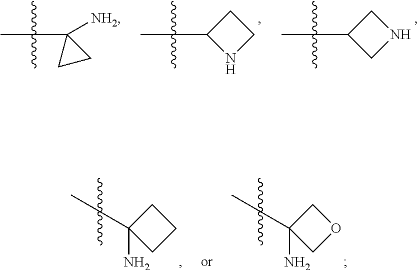 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

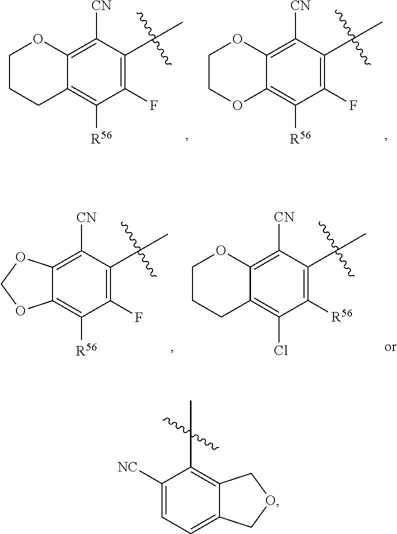

where R⁵⁶ is hydrogen, halogen, C₁-C₆ alkyl, C₁-C₆ haloalkyl, C₁-C₆ alkoxy, or C₁-C₆ haloalkoxy;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

Embodiment 76 provides the compound according to embodiment 74 or 75, wherein A is CH.

Embodiment 77 provides the compound according to embodiment 74 or 75, wherein W is N.

Embodiment 78 provides the compound according to embodiment 74 or 75, wherein W is CH.

Embodiment 79 provides the compound according to any of embodiments 74 or 75, wherein D is —CH₂—NH₂.

Embodiment 80 provides a compound according to embodiment 74 or 75, which is of the formula:

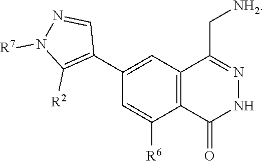

Embodiment 81 provides the compound according to embodiment 74 or 76-80, wherein R² is

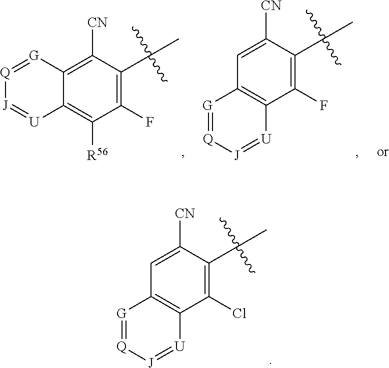

Embodiment 82 provides the compound according to embodiment 81, wherein G, Q, J and U are independently selected from C(H) and C(R⁵).

Embodiment 83 provides the compound according to embodiment 81, wherein G, Q, J and U are independently C(H).

Embodiment 84 provides the compound according to embodiment 81, wherein at least one of G, Q, J, and U is C(R⁵), and the remaining G, Q, J, and U are independently C(H); for example only one of G, Q, J, and U is C(R⁵).

Embodiment 85 provides the compound according to embodiment 81, wherein U is N, and G, Q, and J are independently selected from C(H) and C(R⁵).

Embodiment 86 provides the compound according to embodiment 81, wherein G is N, and Q, J, and U are independently selected from C(H) and C(R⁵).

Embodiment 87 provides the compound according to any one of embodiments 74 or 76-86, wherein R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 88 provides the compound according to any one of embodiments 74 or 76-86, wherein R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

Embodiment 89 provides the compound according to any one of embodiments 74 or 76-86, wherein R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

Embodiment 90 provides the compound according to any one of embodiments 74 or 76-86, wherein R⁵, if present, is halogen, C₁-C₆ alkyl, or C₁-C₆ alkoxy; for example, R⁶ is halogen, C₁-C₃ alkyl, or C₁-C₃ alkoxy.

Embodiment 91 provides the compound according to any one of embodiments 74 or 76-86, wherein R⁵, if present, is chloro, fluoro, methyl, ethyl, methoxy, or ethoxy.

Embodiment 92 provides the compound according to any one of embodiments 74 or 76-86, wherein R⁵⁶ is fluoro, chloro, or methyl.

Embodiment 93 provides the compound according to embodiment 75-80, wherein R² is

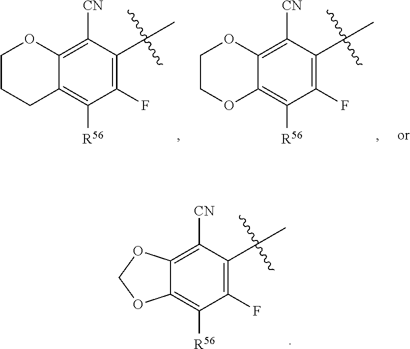

Embodiment 94 provides the compound according to any one of embodiments 75-80 or 93, wherein R⁵⁶ is hydrogen, fluoro, chloro, or methyl.

Embodiment 95 provides the compound according to any of embodiments 74-94, wherein R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 96 provides the compound according to any of embodiments 74-94, wherein R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

Embodiment 97 provides the compound according to any of embodiments 74-94, wherein R⁶ is hydrogen, chloro, fluoro, methyl, ethyl, methoxy, or ethoxy.

Embodiment 98 provides the compound according to any of embodiments 74-94, wherein R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 99 provides the compound according to any of embodiments 74-94, wherein R⁶ is halogen, C₁-C₆ alkyl, or C₁-C₆ alkoxy; for example, R⁶ is halogen, C₁-C₃ alkyl, or C₁-C₃ alkoxy.

Embodiment 100 provides the compound according to any of embodiments 74-94, wherein R⁶ is chloro, fluoro, methyl, ethyl, methoxy, or ethoxy.

Embodiment 101 provides the compound according to any one of embodiments 74-100, wherein R⁷ is methyl.

Embodiment 102 provides the compound according to any one of embodiments 74-100, wherein R⁷ is ethyl.

Embodiment 103 provides the compound according to any one of embodiments 74-100, wherein R⁷ is propyl (e.g., isopropyl).

Embodiment 104 provides the compound according to any one of embodiments 74-100, wherein R⁷ is difluoromethyl or trifluoromethyl.

In another aspect, the disclosure provides a compound of Formula IVA, IVA-1, IVB, IVB-1, IVC or IVC-1 (Embodiment 105):

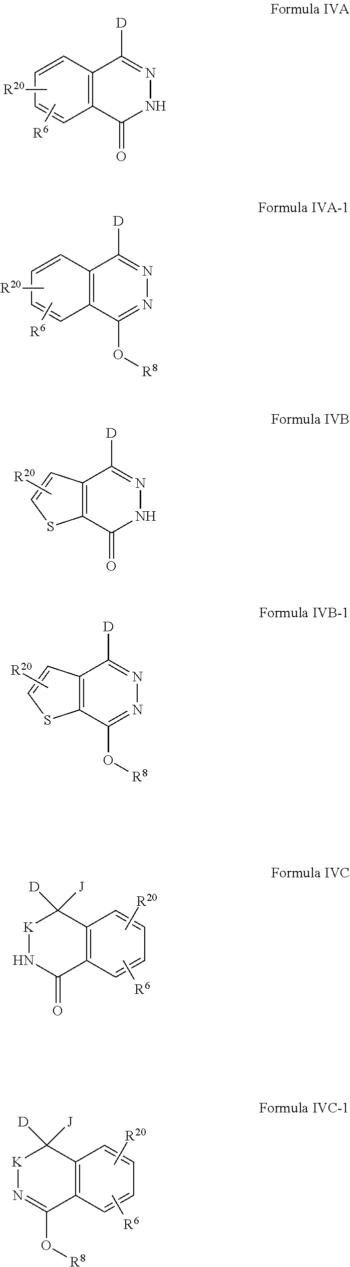

or a pharmaceutically acceptable salt thereof, wherein:
R⁶ is hydrogen, halogen, C1-C3 alkyl, haloalkyl, hydroxy, alkoxy, C1-C3 alkyl-C1-C3alkoxy, N(R⁹)₂, NRC(O)R⁹, C(O)R⁹, C3-C7 heterocycloalkyl;
R²⁰ is hydrogen or a 5-membered heteroaryl group optionally substituted with one or more R²²; provided that not both R⁶ and R²⁰ are hydrogen simultaneously;
each R²² is independently hydroxy, halogen, cyano, cyanomethyl, —(NR⁴)₂, hydroxyalkyl, alkoxy, —SO₂C1-C3alkyl, —X-arylC1-C3alkyl, heteroalkyl, C2-C4 alkynyl, —X-haloalkyl, —X—C1
C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁴;
each X is independently a bond, O, S, —NR⁴— or —NR⁴C(O)—;
each Z is independently a bond, —SO—, —SO₂—, —CH(OH)— or —C(O)—;
each L is independently a bond or C1-C3 alkylene;
each R²⁴ is independently cyano, oxo, halogen, C1-C3 alkyl, hydroxyalkyl, alkoxy, —X— haloalkyl, —Z-cycloalkyl, —X-arC1-C3alkyl, X-arC1-C3alkyl substituted with cyano, —X-L-cycloalkyl, —X-L-heteroaryl optionally substituted with one or more C1-C3alkyl or oxo, or —X-aryl;

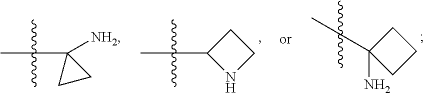 [SMILES: *C1(N)CC1 ; *C1CCN1 ; *C1(N)CCC1]

D is —CO₂R³⁰, (C(R⁹)₂)₁₋₂—NHR¹¹,
J is hydrogen or C1-C6 alkyl;
K is a bond or C(R²⁶)₂ where each R²⁶ is independently hydrogen or C1-C3alkyl;
each R⁴ is independently hydrogen or C1-C3 alkyl;
R⁸ is C1-C3 alkyl;
each R⁹ is independently H or C1-C3 alkyl, halogen or haloalkyl;
R³⁰ is hydrogen or C1-C6 alkyl;
R¹⁰ is H, C1-C3 alkyl, halogen, haloalkyl, or C3-C7 heterocycloalkyl; and
R¹¹ represents hydrogen or —C(O)R¹², where R¹² is hydrogen or C1-C3 alkyl.

Embodiment 106 provides the compound according to embodiment 105, having Formula IVA and wherein R²⁰ is hydrogen.

Embodiment 107 provides the compound according to embodiment 105, having Formula IVA and wherein R²⁰ is pyrazolyl or imidazolyl each of which is optionally substituted with one or more R²².

Embodiment 108 provides the compound according to embodiment 105, having Formula IVB and wherein R²¹ is pyrazolyl or imidazolyl each of which is optionally substituted with one or more R²².

Embodiment 109 provides the compound according to embodiment 105, having Formula IVC wherein K is a bond, J is hydrogen, and R²⁰ is pyrazolyl or imidazolyl each of which is optionally substituted with one or more R²².

Embodiment 110 provides the compound according to embodiment 105, having Formula IVC wherein K is CH₂b, J is hydrogen or methyl, and R²⁰ is pyrazolyl or imidazolyl each of which is optionally substituted with one or more R²².

Embodiments of the invention further include those wherein there is provided a compound which is:

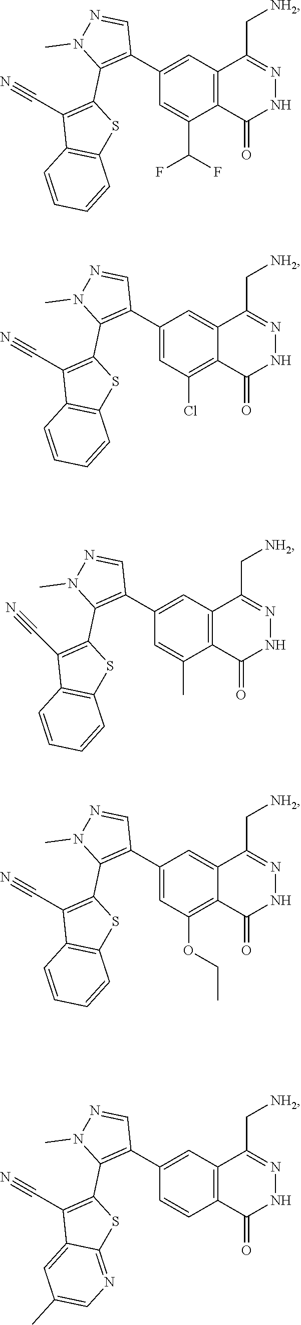 [SMILES: Cn1ncc(-c2cc(C(F)F)c3c(=O)[nH]nc(CN)c3c2)c1-c1sc2ccccc2c1C#N ; Cn1ncc(-c2cc(Cl)c3c(=O)[nH]nc(CN)c3c2)c1-c1sc2ccccc2c1C#N ; Cc1cc(-c2cnn(C)c2-c2sc3ccccc3c2C#N)cc2c(CN)n[nH]c(=O)c12 ; CCOc1cc(-c2cnn(C)c2-c2sc3ccccc3c2C#N)cc2c(CN)n[nH]c(=O)c12 ; Cc1cnc2sc(-c3c(-c4ccc5c(=O)[nH]nc(CN)c5c4)cnn3C)c(C#N)c2c1]

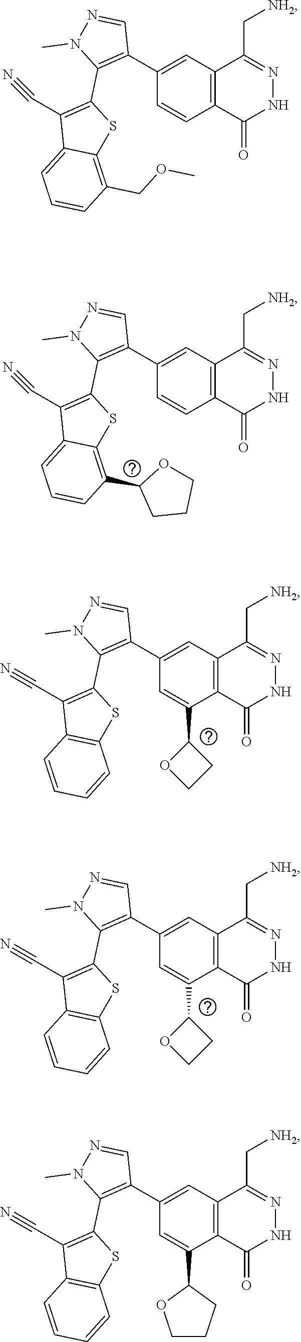 [SMILES: COCc1cccc2c(C#N)c(-c3c(-c4ccc5c(=O)[nH]nc(CN)c5c4)cnn3C)sc12 ; Cn1ncc(-c2ccc3c(=O)[nH]nc(CN)c3c2)c1-c1sc2c([C@@H]3CCCO3)cccc2c1C#N ; Cn1ncc(-c2cc([C@H]3CCO3)c3c(=O)[nH]nc(CN)c3c2)c1-c1sc2ccccc2c1C#N ; Cn1ncc(-c2cc([C@@H]3CCO3)c3c(=O)[nH]nc(CN)c3c2)c1-c1sc2ccccc2c1C#N ; Cn1ncc(-c2cc([C@H]3CCCO3)c3c(=O)[nH]nc(CN)c3c2)c1-c1sc2ccccc2c1C#N]

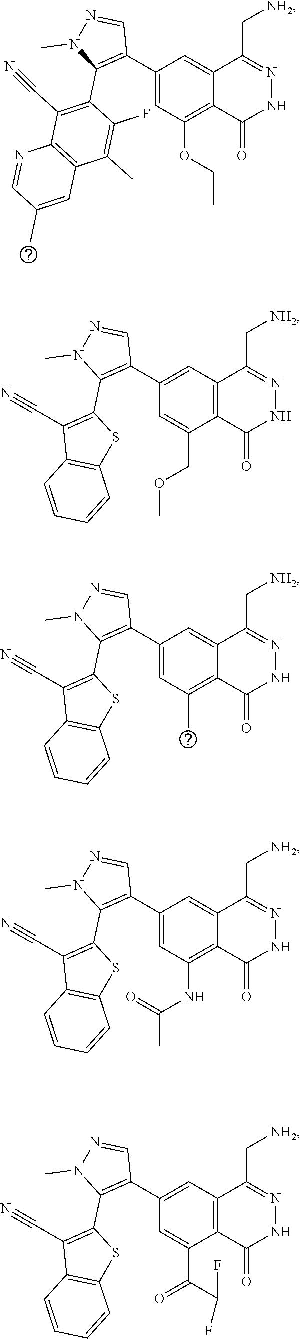

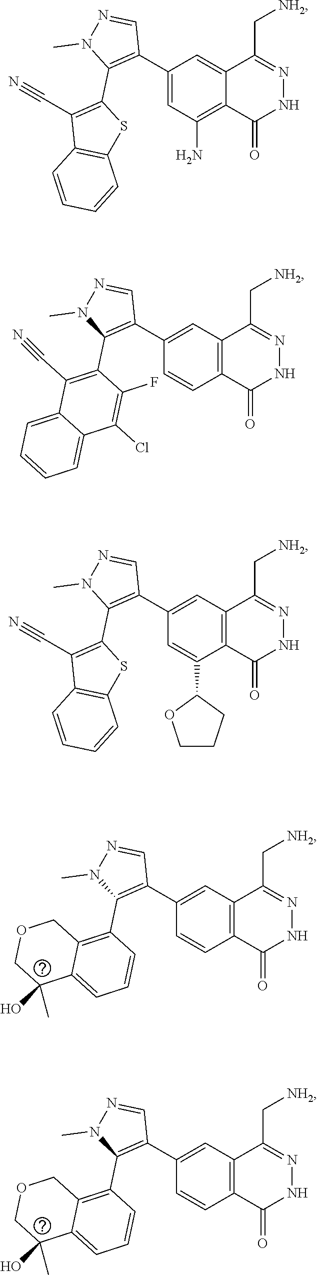

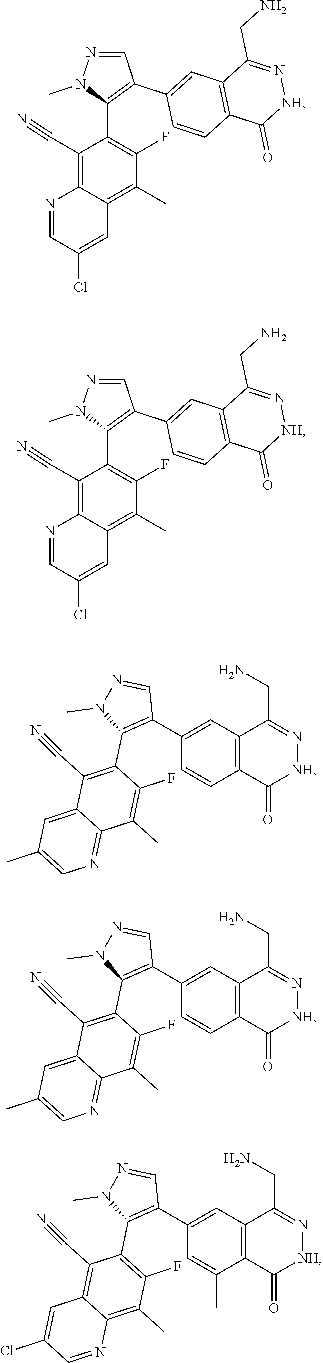

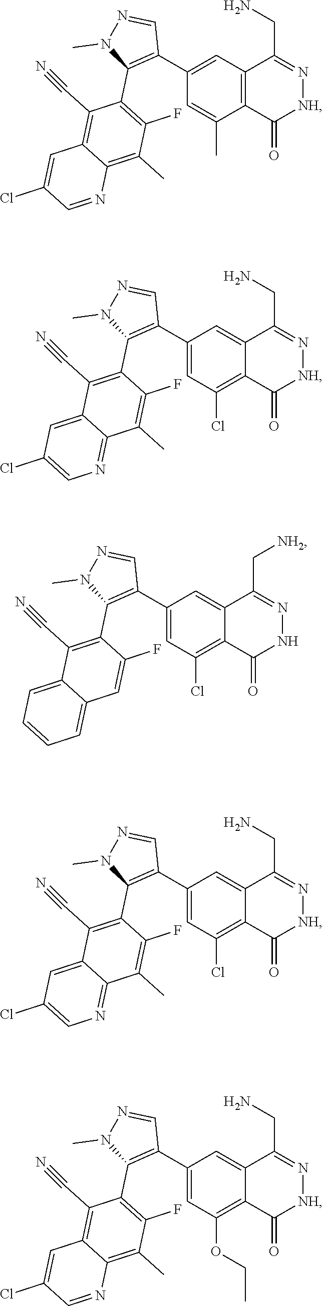

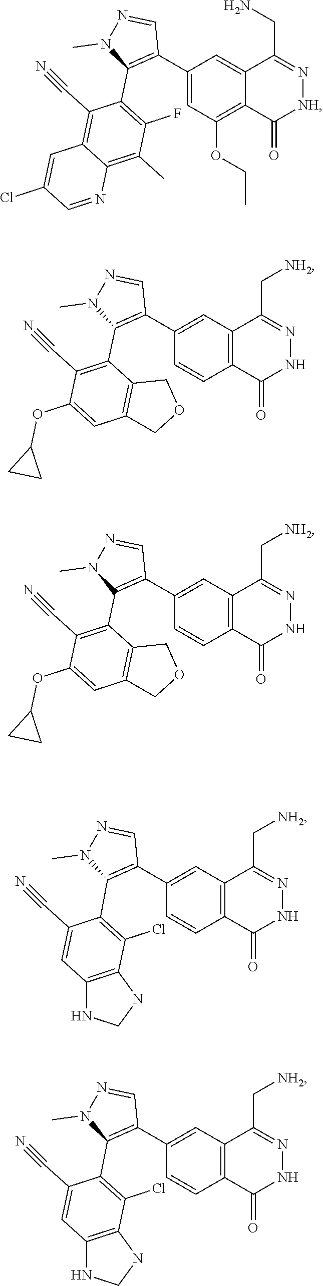

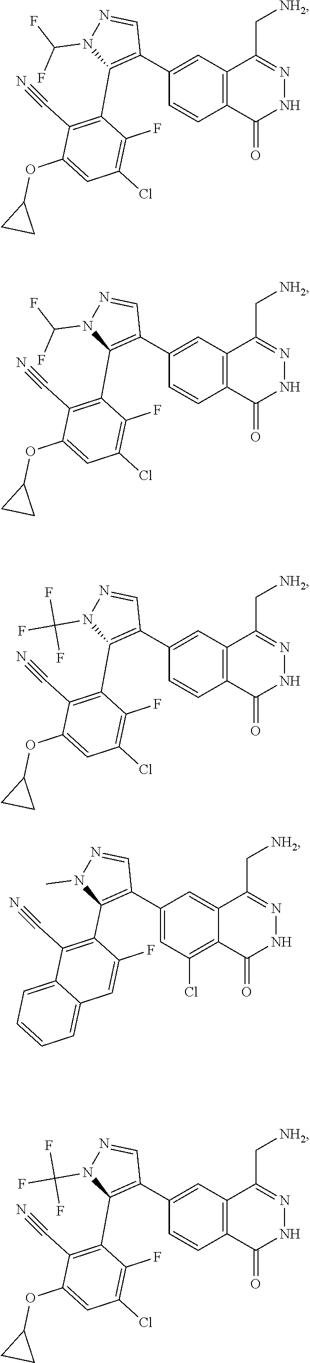

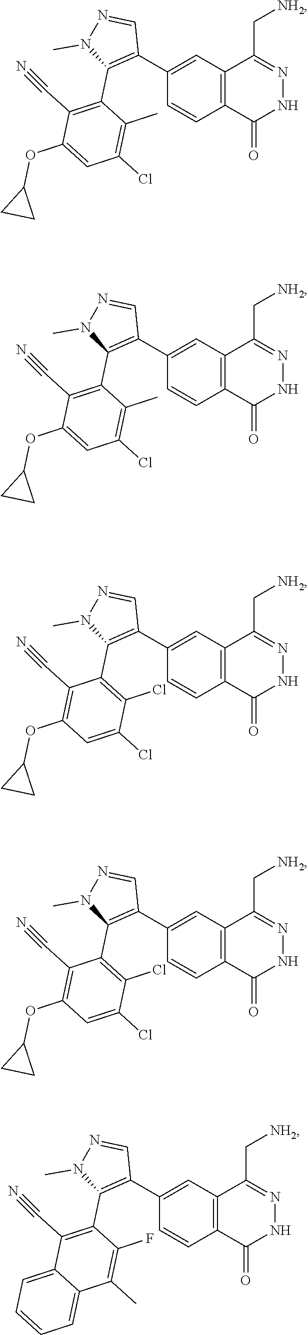

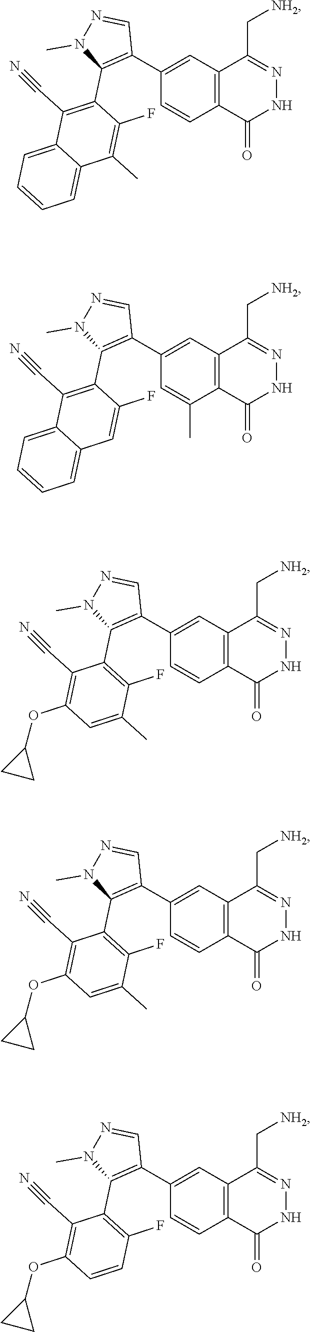

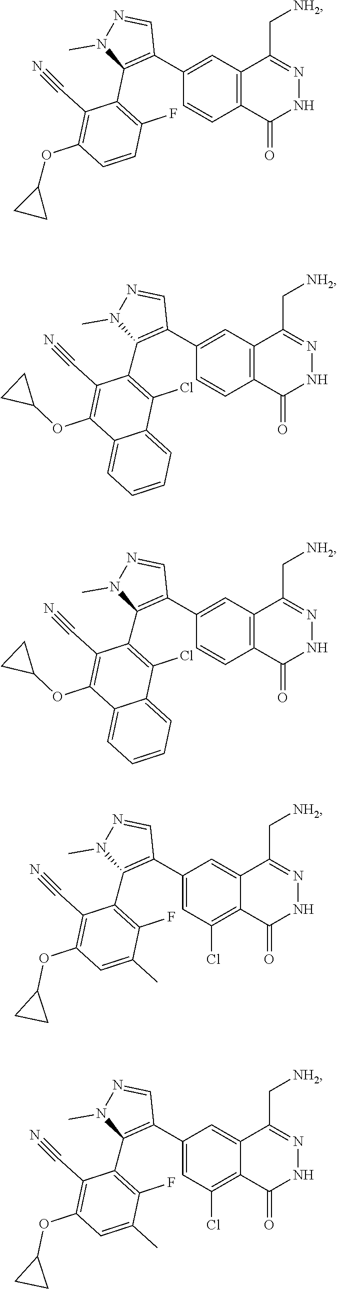

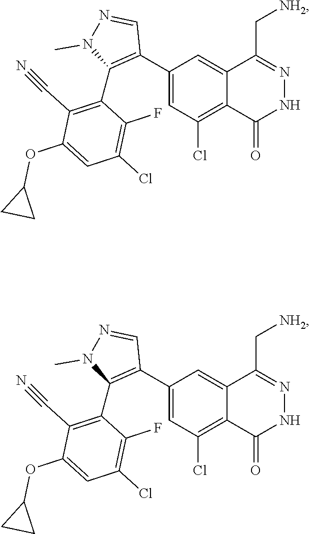

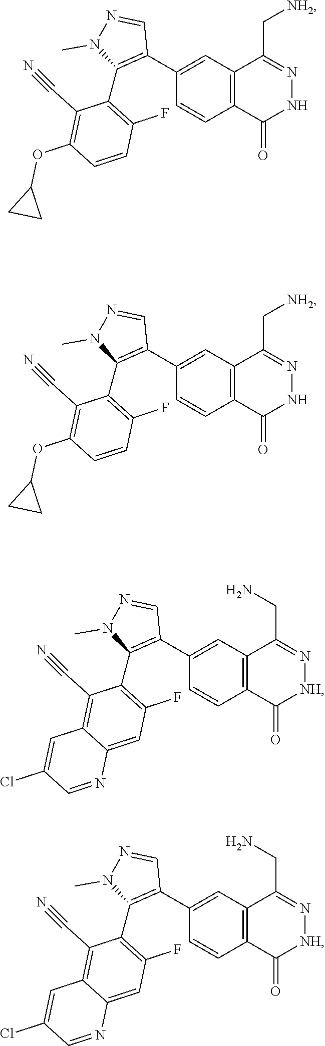

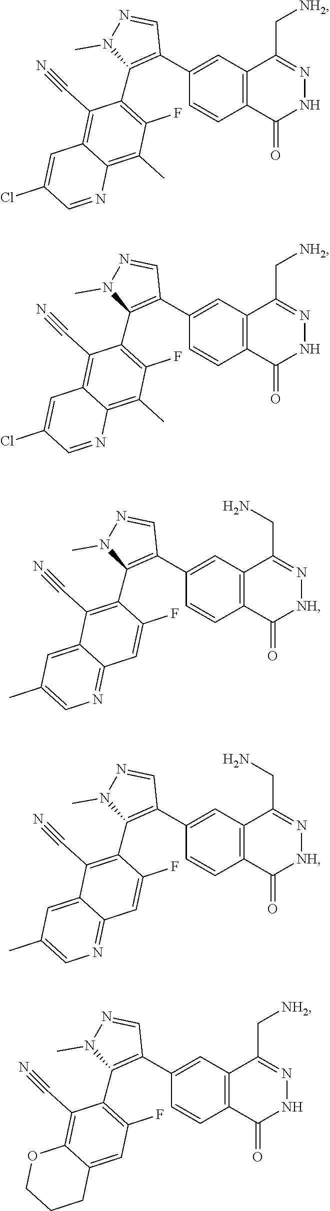

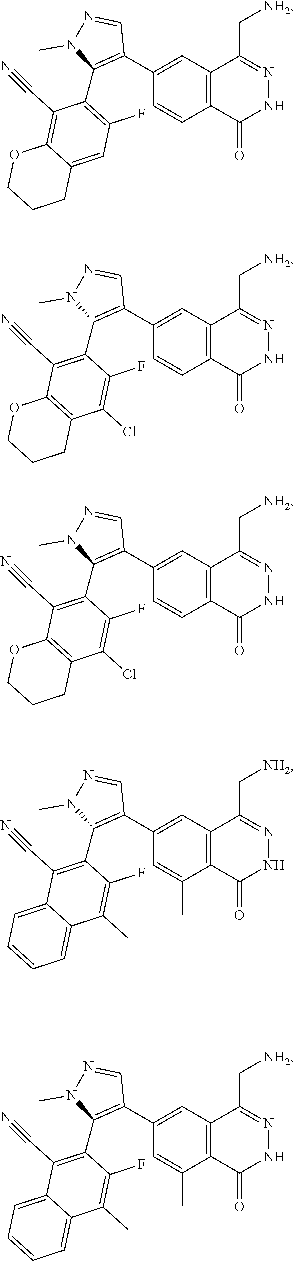

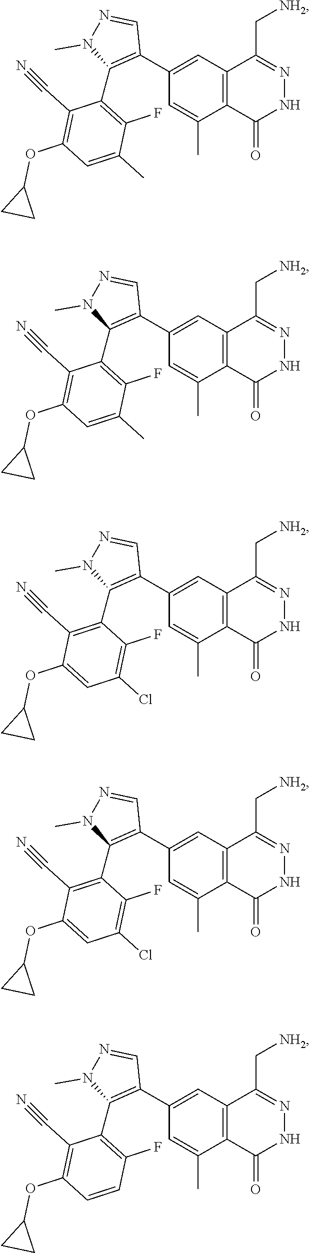

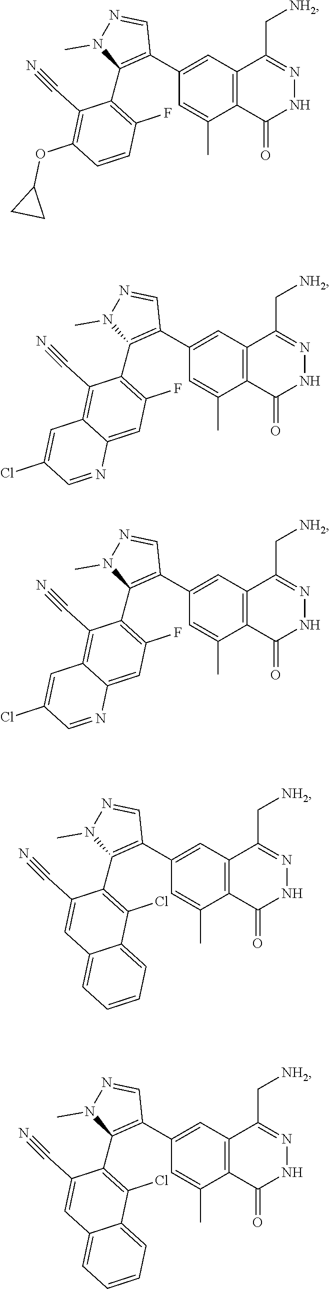

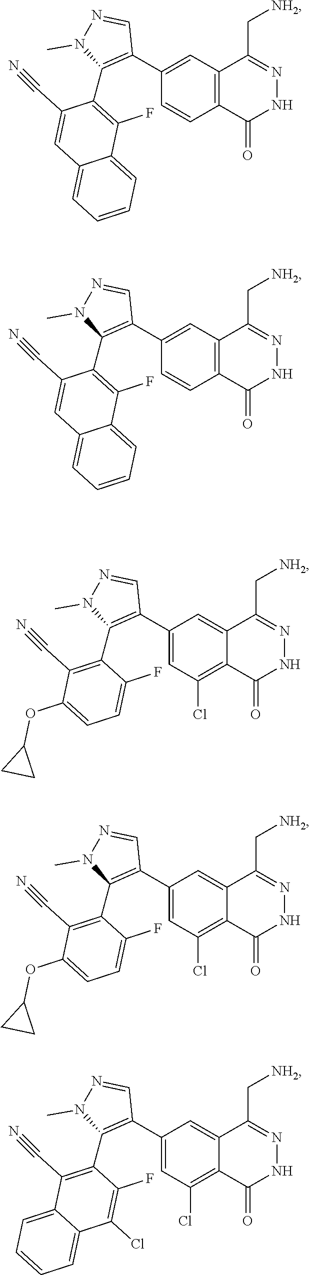

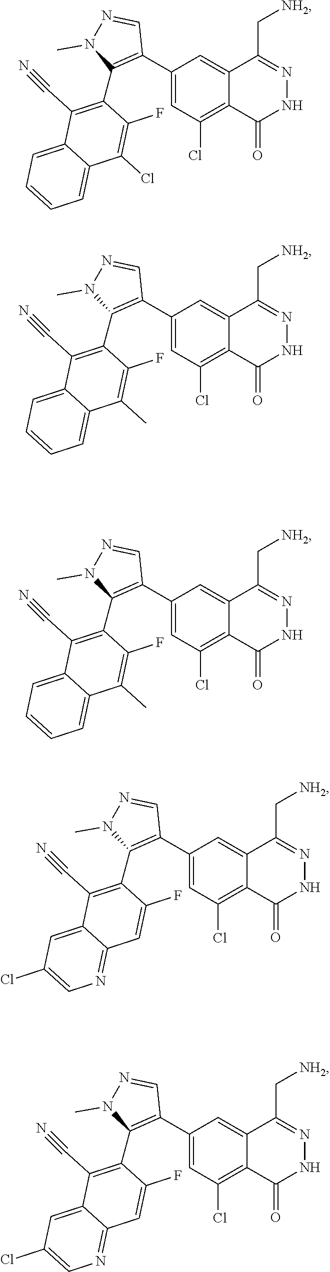

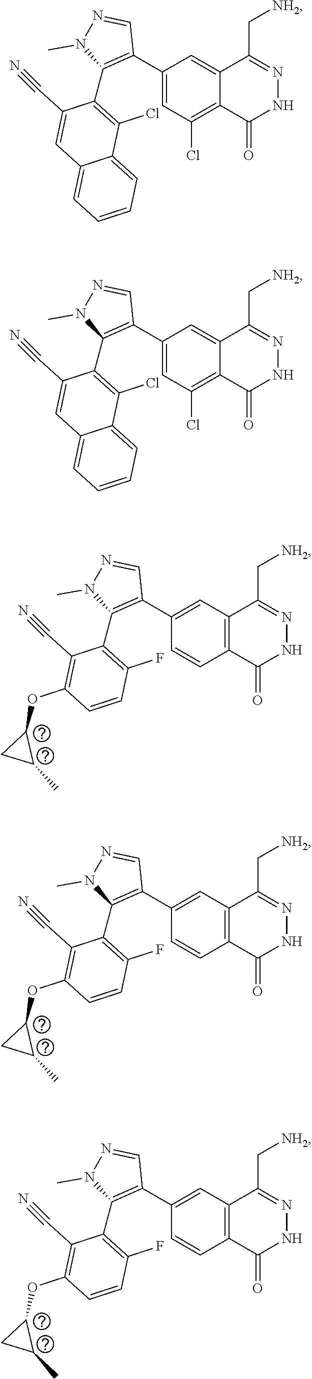

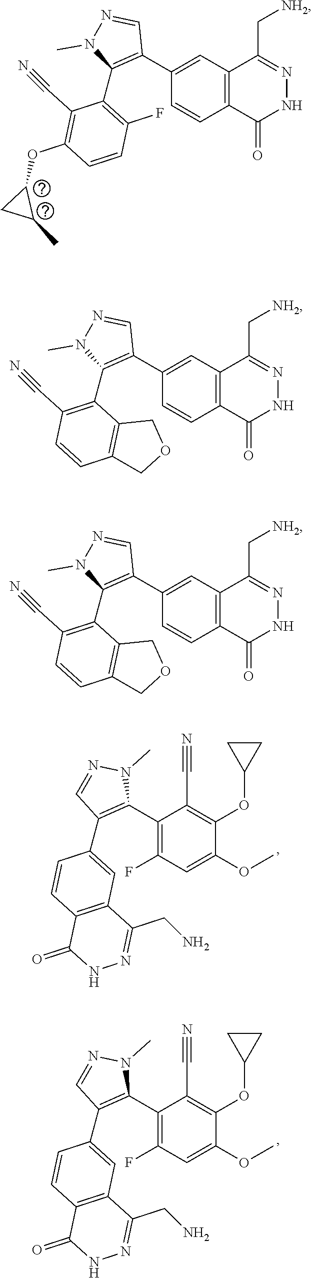

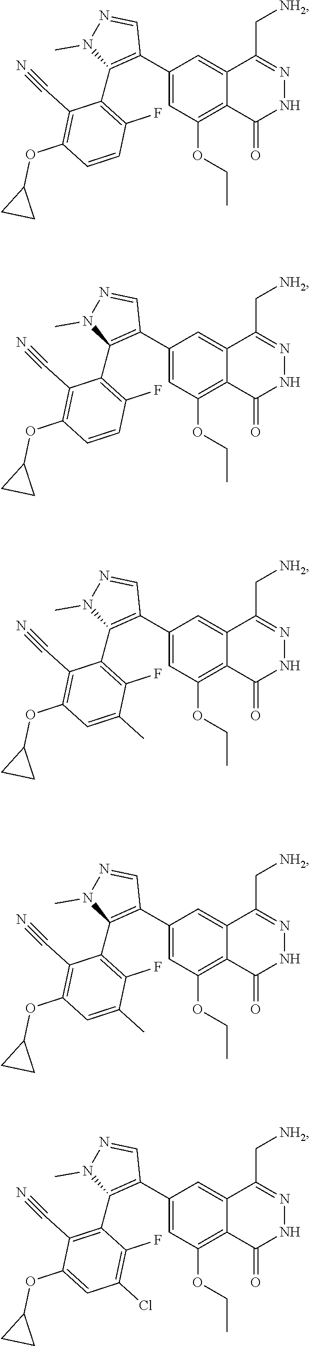

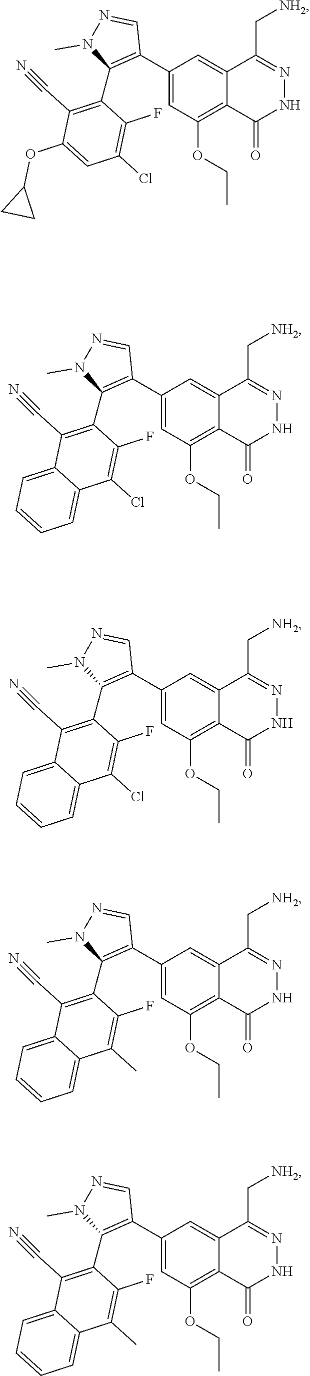

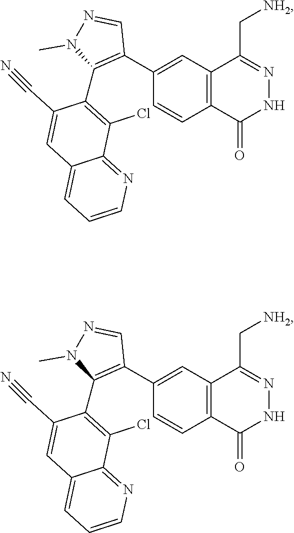

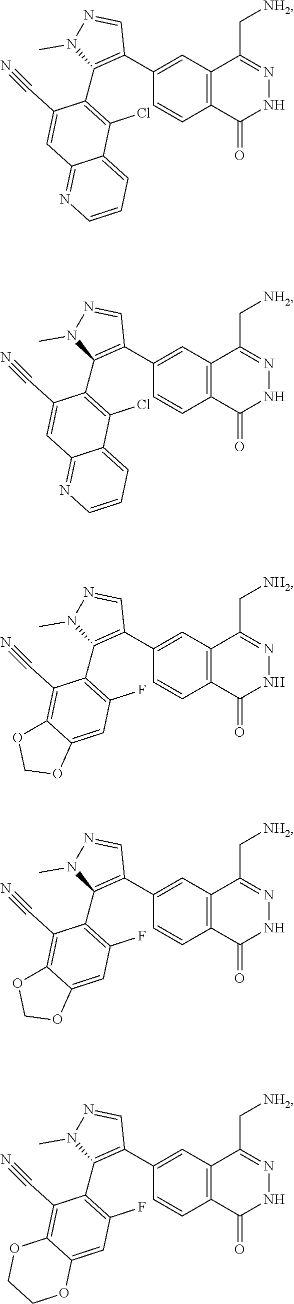

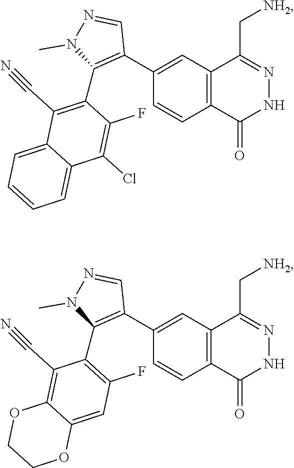

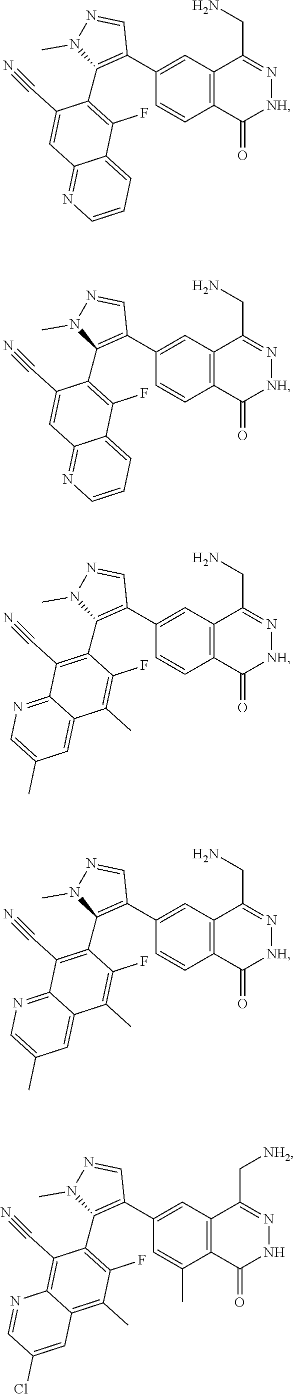

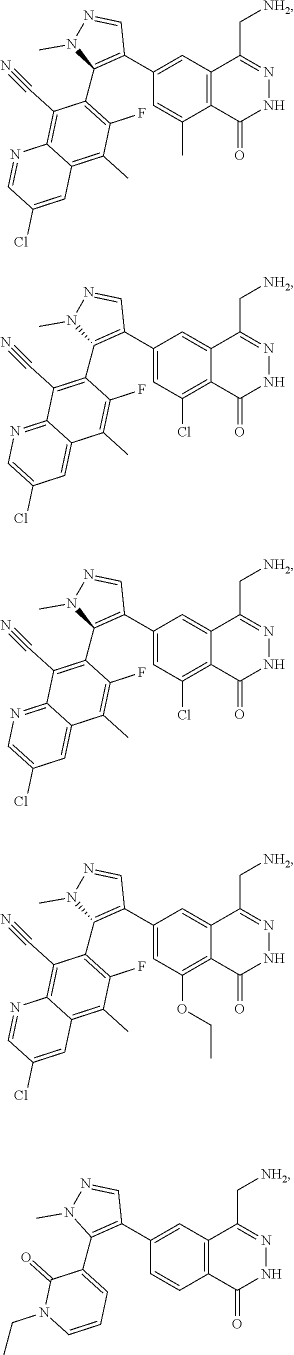

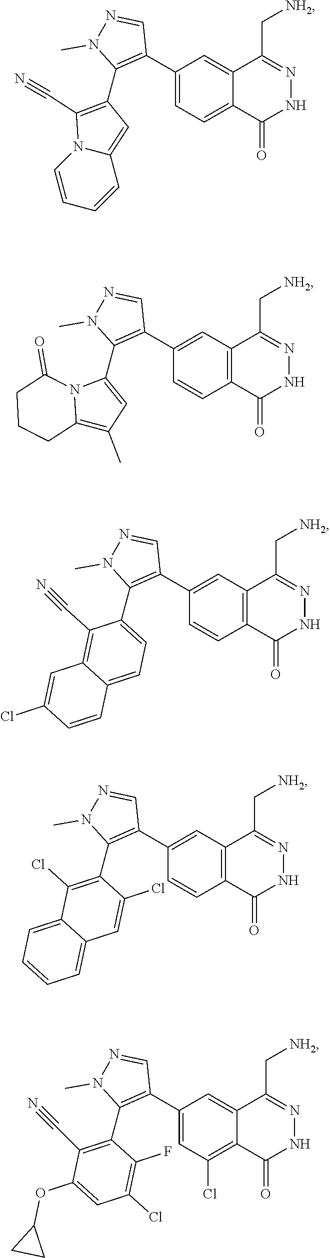 [SMILES: Cn1ncc(-c2ccc3c(=O)[nH]nc(CN)c3c2)c1-c1cc2ccccn2c1C#N ; Cc1cc(-c2c(-c3ccc4c(=O)[nH]nc(CN)c4c3)cnn2C)n2c1CCCC2=O ; Cn1ncc(-c2ccc3c(=O)[nH]nc(CN)c3c2)c1-c1ccc2ccc(Cl)cc2c1C#N ; Cn1ncc(-c2ccc3c(=O)[nH]nc(CN)c3c2)c1-c1c(Cl)cc2ccccc2c1Cl ; Cn1ncc(-c2cc(Cl)c3c(=O)[nH]nc(CN)c3c2)c1-c1c(F)c(Cl)cc(OC2CC2)c1C#N]

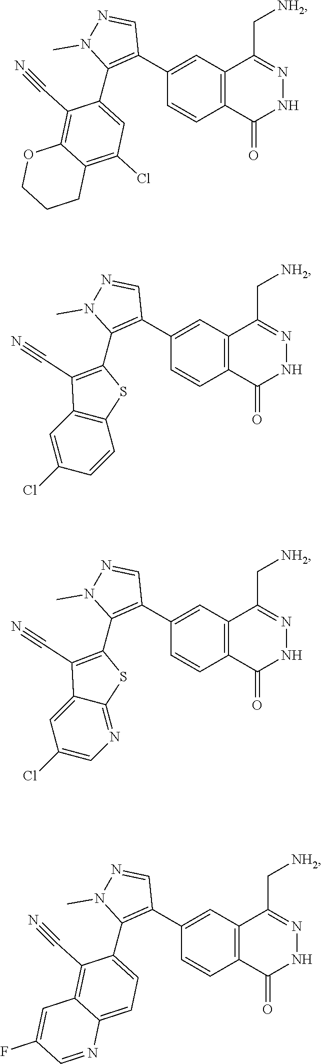 [SMILES: Cn1ncc(-c2ccc3c(=O)[nH]nc(CN)c3c2)c1-c1cc(Cl)c2c(c1C#N)OCCC2 ; Cn1ncc(-c2ccc3c(=O)[nH]nc(CN)c3c2)c1-c1sc2ccc(Cl)cc2c1C#N ; Cn1ncc(-c2ccc3c(=O)[nH]nc(CN)c3c2)c1-c1sc2ncc(Cl)cc2c1C#N ; Cn1ncc(-c2ccc3c(=O)[nH]nc(CN)c3c2)c1-c1ccc2ncc(F)cc2c1C#N]

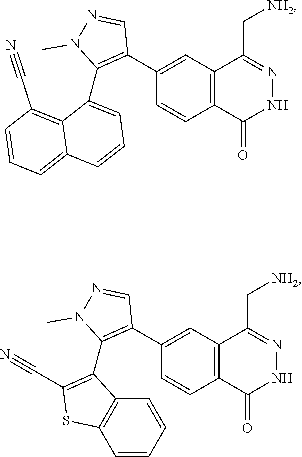 [SMILES: Cn1ncc(-c2ccc3c(=O)[nH]nc(CN)c3c2)c1-c1cccc2cccc(C#N)c12 ; Cn1ncc(-c2ccc3c(=O)[nH]nc(CN)c3c2)c1-c1c(C#N)sc2ccccc12]

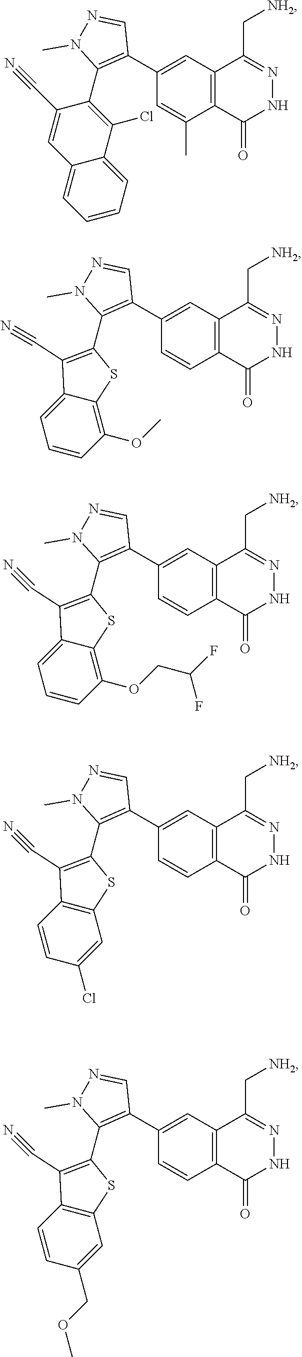

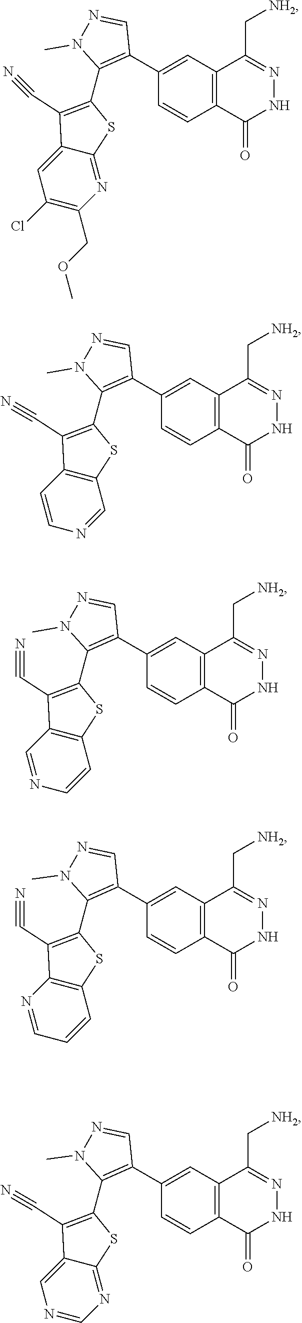

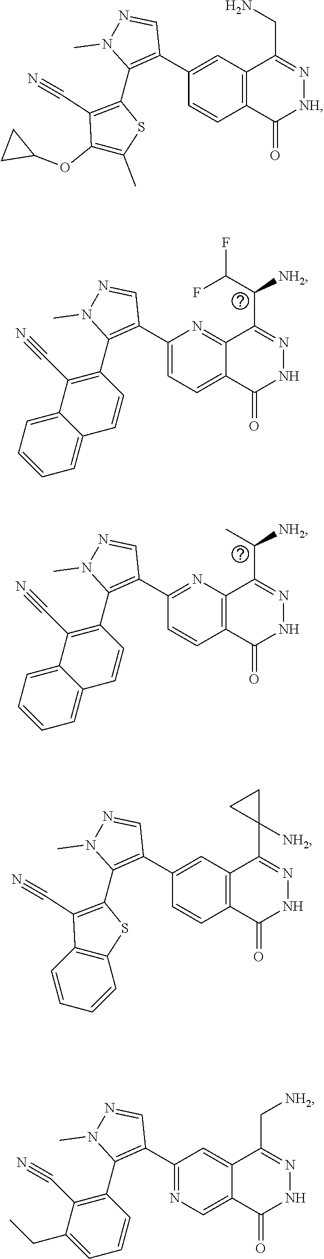 [SMILES: Cc1sc(-c2c(-c3ccc4c(=O)[nH]nc(CN)c4c3)cnn2C)c(C#N)c1OC1CC1 ; Cn1ncc(-c2ccc3c(=O)[nH]nc([C@H](N)C(F)F)c3n2)c1-c1ccc2ccccc2c1C#N ; C[C@@H](N)c1n[nH]c(=O)c2ccc(-c3cnn(C)c3-c3ccc4ccccc4c3C#N)nc12 ; Cn1ncc(-c2ccc3c(=O)[nH]nc(C4(N)CC4)c3c2)c1-c1sc2ccccc2c1C#N ; CCc1cccc(-c2c(-c3cc4c(CN)n[nH]c(=O)c4cn3)cnn2C)c1C#N]

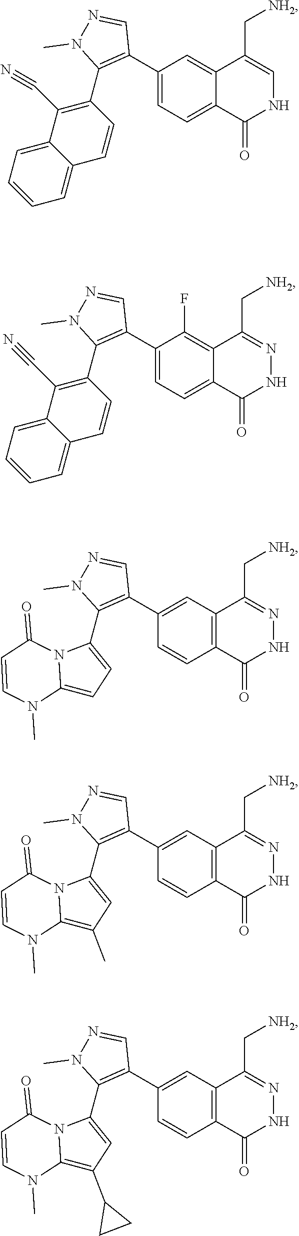 [SMILES: Cn1ncc(-c2ccc3c(=O)[nH]cc(CN)c3c2)c1-c1ccc2ccccc2c1C#N ; Cn1ncc(-c2ccc3c(=O)[nH]nc(CN)c3c2F)c1-c1ccc2ccccc2c1C#N ; Cn1ncc(-c2ccc3c(=O)[nH]nc(CN)c3c2)c1-c1ccc2n(C)ccc(=O)n12 ; Cc1cc(-c2c(-c3ccc4c(=O)[nH]nc(CN)c4c3)cnn2C)n2c(=O)ccn(C)c12 ; Cn1ncc(-c2ccc3c(=O)[nH]nc(CN)c3c2)c1-c1cc(C2CC2)c2n(C)ccc(=O)n12]

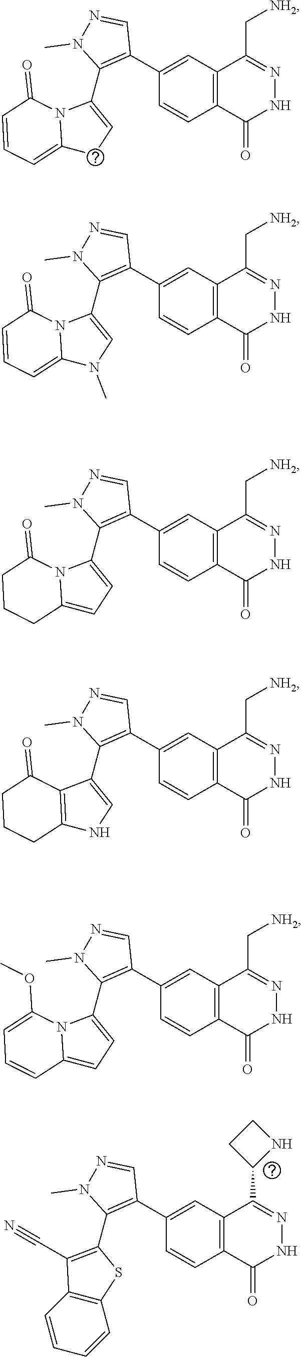 [SMILES: Cn1ncc(-c2ccc3c(=O)[nH]nc(CN)c3c2)c1C1=CCc2cccc(=O)n21 ; Cn1ncc(-c2ccc3c(=O)[nH]nc(CN)c3c2)c1-c1cn(C)c2cccc(=O)n12 ; Cn1ncc(-c2ccc3c(=O)[nH]nc(CN)c3c2)c1-c1ccc2n1C(=O)CCC2 ; Cn1ncc(-c2ccc3c(=O)[nH]nc(CN)c3c2)c1-c1c[nH]c2c1C(=O)CCC2 ; COc1cccc2ccc(-c3c(-c4ccc5c(=O)[nH]nc(CN)c5c4)cnn3C)n12 ; Cn1ncc(-c2ccc3c(=O)[nH]nc([C@@H]4CCN4)c3c2)c1-c1sc2ccccc2c1C#N]

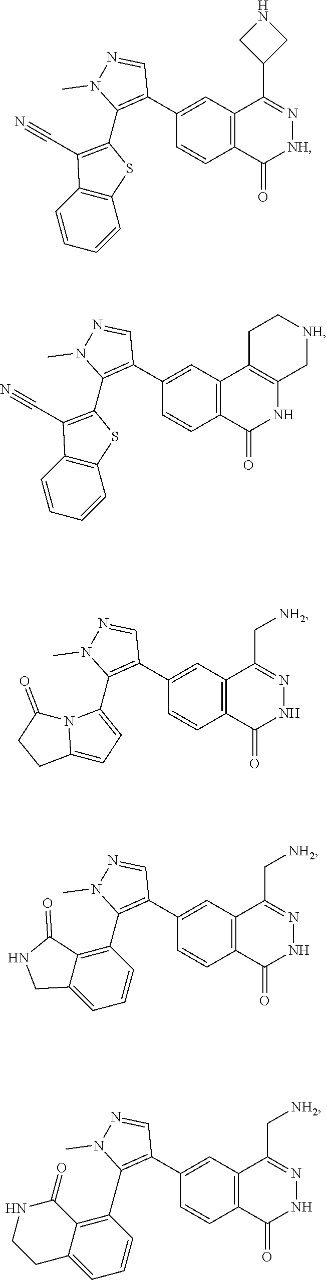 [SMILES: Cn1ncc(-c2ccc3c(=O)[nH]nc(C4CNC4)c3c2)c1-c1sc2ccccc2c1C#N ; Cn1ncc(-c2ccc3c(=O)[nH]c4c(c3c2)CCNC4)c1-c1sc2ccccc2c1C#N ; Cn1ncc(-c2ccc3c(=O)[nH]nc(CN)c3c2)c1-c1ccc2n1C(=O)CC2 ; Cn1ncc(-c2ccc3c(=O)[nH]nc(CN)c3c2)c1-c1cccc2c1C(=O)NC2 ; Cn1ncc(-c2ccc3c(=O)[nH]nc(CN)c3c2)c1-c1cccc2c1C(=O)NCC2]

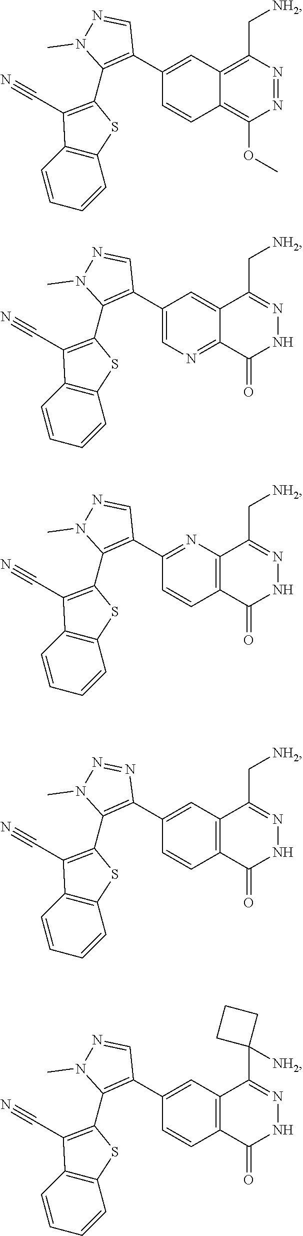

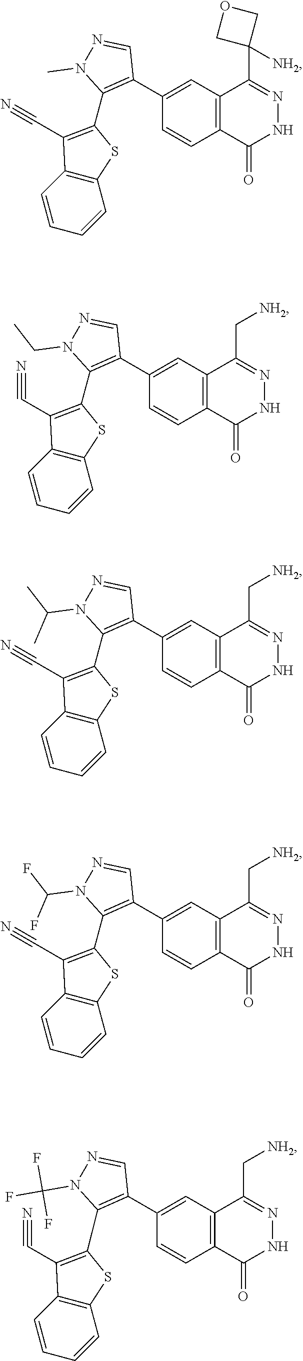 [SMILES: Cn1ncc(-c2ccc3c(=O)[nH]nc(C4(N)COC4)c3c2)c1-c1sc2ccccc2c1C#N ; CCn1ncc(-c2ccc3c(=O)[nH]nc(CN)c3c2)c1-c1sc2ccccc2c1C#N ; CC(C)n1ncc(-c2ccc3c(=O)[nH]nc(CN)c3c2)c1-c1sc2ccccc2c1C#N ; N#Cc1c(-c2c(-c3ccc4c(=O)[nH]nc(CN)c4c3)cnn2C(F)F)sc2ccccc12 ; N#Cc1c(-c2c(-c3ccc4c(=O)[nH]nc(CN)c4c3)cnn2C(F)(F)F)sc2ccccc12]

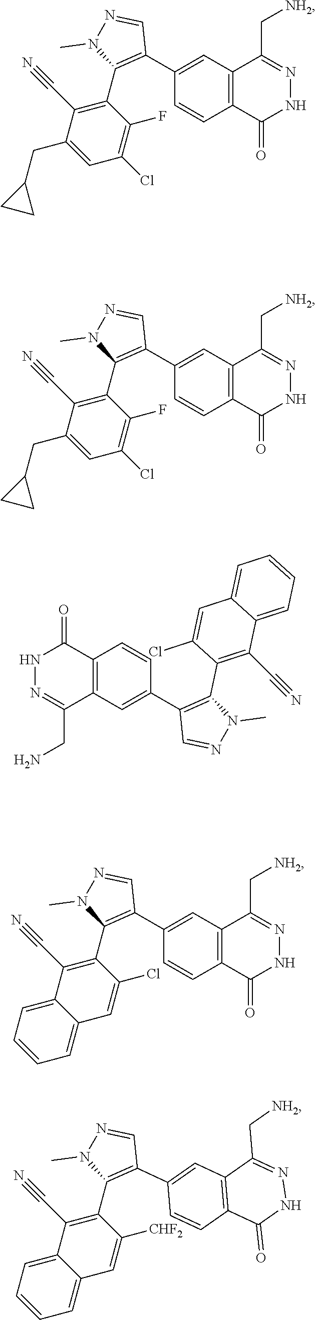

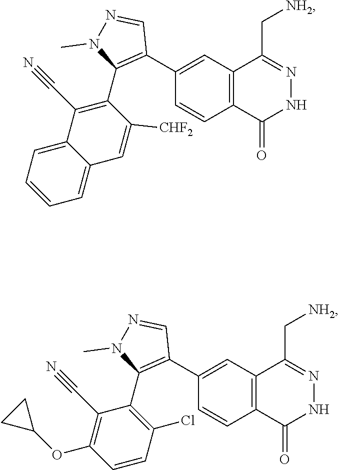

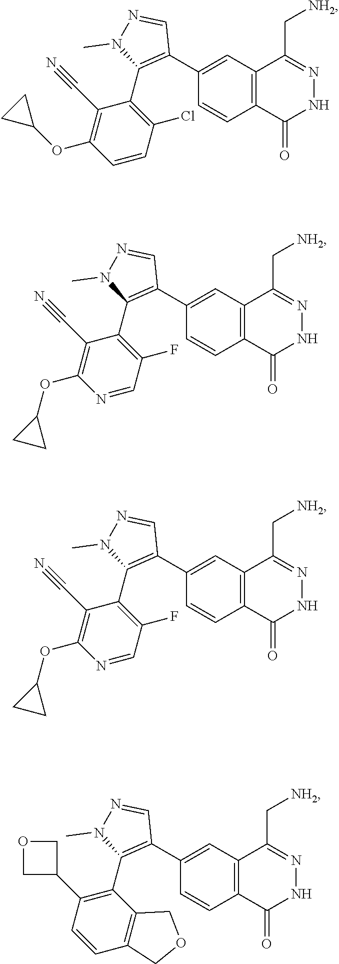

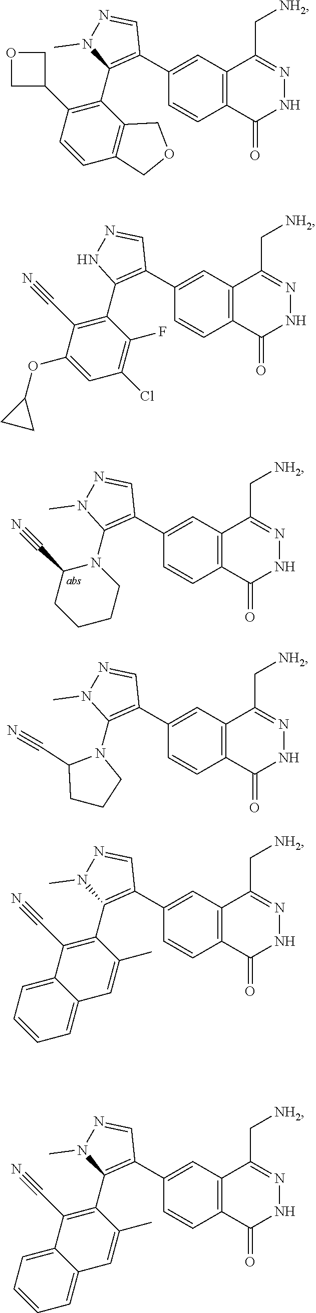

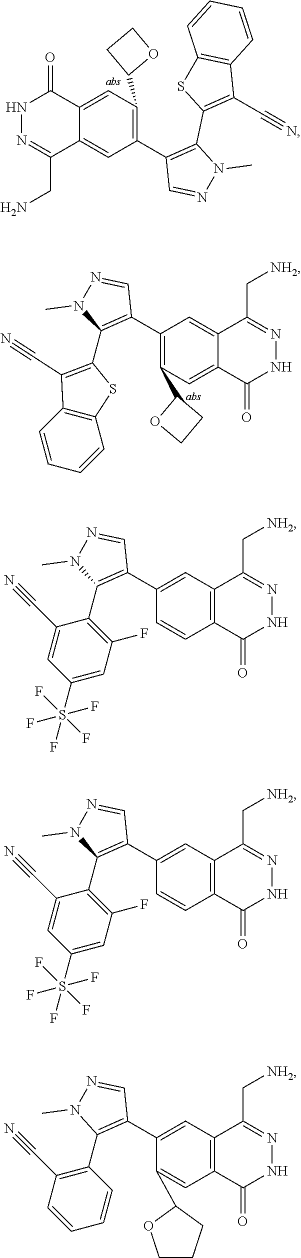

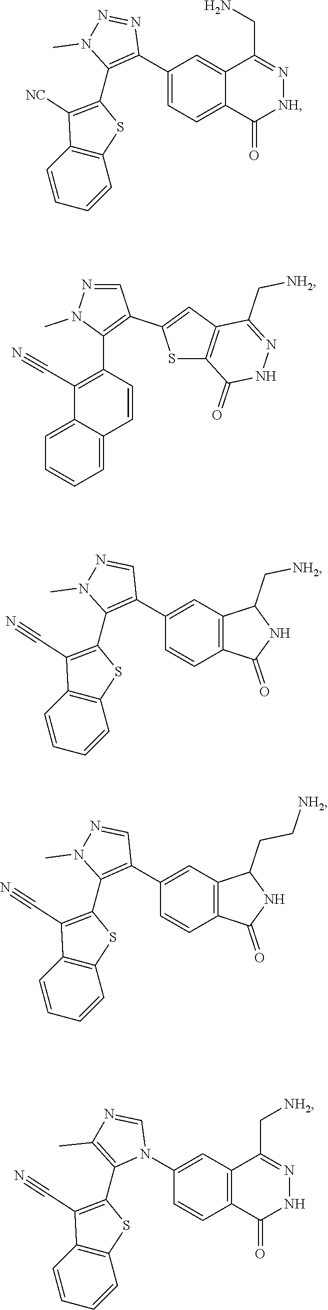 [SMILES: Cn1nnc(-c2ccc3c(=O)[nH]nc(CN)c3c2)c1-c1sc2ccccc2c1C#N ; Cn1ncc(-c2cc3c(CN)n[nH]c(=O)c3s2)c1-c1ccc2ccccc2c1C#N ; Cn1ncc(-c2ccc3c(c2)C(CN)NC3=O)c1-c1sc2ccccc2c1C#N ; Cn1ncc(-c2ccc3c(c2)C(CCN)NC3=O)c1-c1sc2ccccc2c1C#N ; Cc1ncn(-c2ccc3c(=O)[nH]nc(CN)c3c2)c1-c1sc2ccccc2c1C#N]

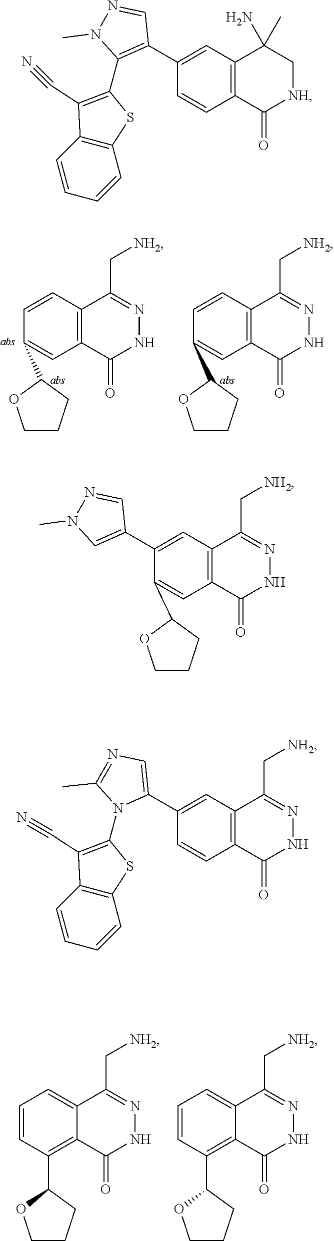

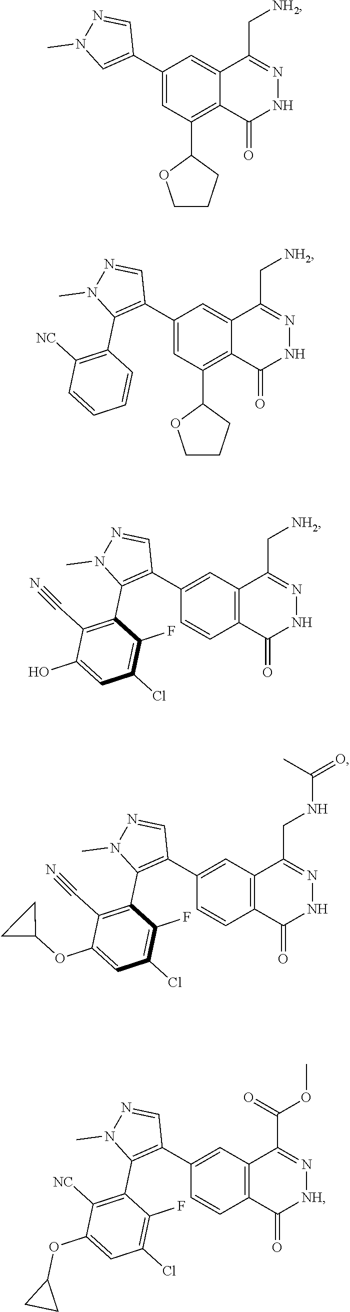

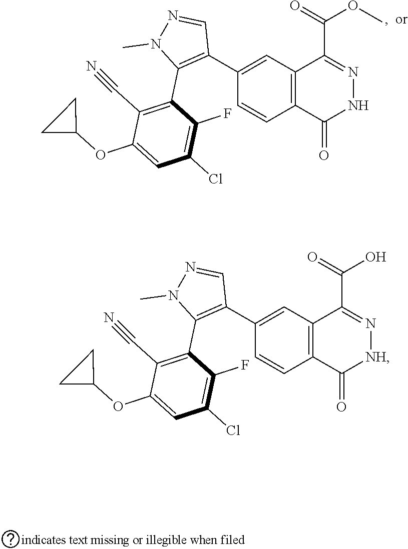

or a pharmaceutically acceptable salt thereof.

In some embodiments of the invention there is provided a pharmaceutical composition, comprising a therapeutically effective amount of a compound as described herein, or a pharmaceutically acceptable salt or solvate thereof, and a pharmaceutically acceptable excipient.

In some embodiments of the invention there is provided a method for inhibiting PRMT5 activity in a cell, comprising contacting the cell in which inhibition of PRMT5 activity is desired with an effective amount of a compound as described herein, or a pharmaceutically acceptable salt or solvate thereof, or a pharmaceutical composition as described herein.

In some embodiments of the invention there is provided a method for treating cancer comprising administering to a patient having cancer a therapeutically effective amount of a compound as described herein, or a pharmaceutically acceptable salt or solvate thereof, alone or combined with a pharmaceutically acceptable carrier, excipient or diluents.

### Pharmaceutical Compositions

In another aspect, the invention provides pharmaceutical compositions comprising a PRMT5 inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In yet another aspect, the invention provides for methods for inhibiting PRMT5 activity in a cell, comprising contacting the cell in which inhibition of PRMT5 activity is desired in vitro with an effective amount of a compound of Formula (I), Formula (I-A), Formula (I-B), Formula (I-C), Formula IIA, Formula IIA-1, Formula IIB, Formula IIB-1, Formula IC, Formula IIC-1, Formula IVA, Formula IVA-1, Formula IVB, Formula IVB-1, Formula IVC, and/or Formula IVC-1, pharmaceutically acceptable salts thereof or pharmaceutical compositions containing the compound or pharmaceutically acceptable salt thereof. In one embodiment, the cell is an MTAP-deficient cell.

The compositions and methods provided herein are particularly deemed useful for inhibiting PRMT5 activity in a cell in vivo. In one embodiment, a cell in which inhibition of PRMT5 activity is desired is contacted in vivo with a therapeutically effective amount of a compound of Formula (I), Formula (I-A), Formula (I-B), Formula (I-C), Formula IIA, Formula IIA-1, Formula IIB, Formula IIB-1, Formula IIC, Formula IIC-1, Formula IVA, Formula IVA-1, Formula IVB, Formula IVB-1, Formula IVC, and/or Formula IVC-1, or a pharmaceutically acceptable salt thereof, to negatively modulate the activity of PRMT5. In other embodiments, a therapeutically effective amount of pharmaceutically acceptable salt or pharmaceutical compositions containing the compound of Formula (I), Formula (I-A), Formula (I-B), Formula (I-C), Formula IIA, Formula IIA-1, Formula IIB, Formula IIB-1, Formula IIC, Formula IIC-1, Formula IVA, Formula IVA-1, Formula IVB, Formula IVB-1, Formula IVC, and/or Formula IVC-1 may be used. In one embodiment, the cell is an MTAP-deficient cell. In one embodiment, the negatively modulating the activity of PRMT5 occurs in the presence of bound MTA.

By negatively modulating the activity of PRMT5, particularly in cases for cells that lack MTAP activity, the methods are designed to inhibit PRMT5 activity to block cellular proliferation. The cells may be contacted in a single dose or multiple doses in accordance with a particular treatment regimen to affect the desired negative modulation of PRMT5. The degree PRMT5 inhibition may be monitored in vitro against the enzyme in the presence and absence of MTA and in the cell using well known methods, including those described in Example B below, to assess the effectiveness of treatment and dosages.

In another aspect, methods of treating cancer comprising administering to a patient having cancer a therapeutically effective amount of a compound of Formula (I), Formula (I-A), Formula (I-B), Formula (I-C), Formula IIA, Formula IIA-1, Formula IIB, Formula IIB-1, Formula IIC, Formula IIC-1, Formula IVA, Formula IVA-1, Formula IVB, Formula IVB-1, Formula IVC, and/or Formula IVC-1, pharmaceutically acceptable salts thereof or pharmaceutical compositions comprising the compound or pharmaceutically acceptable salts thereof are provided. In one embodiment, the cancer is an MTAP-associated cancer.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is an MTAP-associated cancer selected from hepatocellular carcinoma, breast cancer, skin cancer, bladder cancer, liver cancer, pancreatic cancer, and head and neck cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### General Reaction Scheme, Intermediates and Examples

### General Reaction Schemes

The compounds of the present invention may be prepared using commercially available reagents and intermediates in the synthetic methods and reaction schemes described herein, or may be prepared using other reagents and conventional methods well known to those skilled in the art.

For instance, intermediates for preparing compounds and compounds of Formula (I), Formula (I-A), Formula (I-B), Formula (I-C), Formula IIA, Formula IIA-1, Formula IIB, Formula IIB-1, Formula IC, Formula IIC-1, Formula IVA, Formula IVA-1, Formula IVB, Formula IVB-1, Formula IVC, and/or Formula IVC-1 of the present invention may be prepared according to General Reaction Schemes I-XVI. Note that although the below General Reaction Schemes I-XVI refer to Formula (I), these schemes apply equally to Formula (II) and Formula (IV)

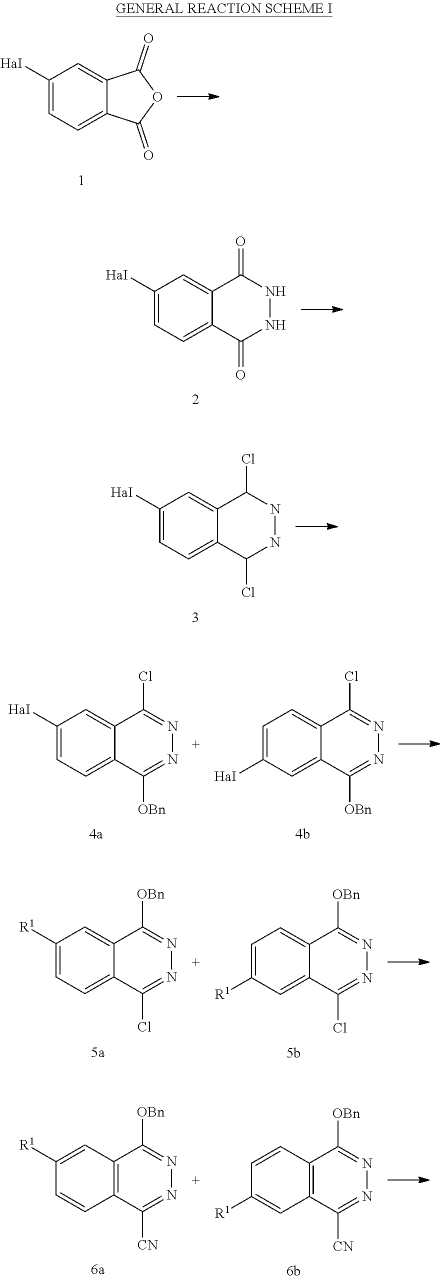

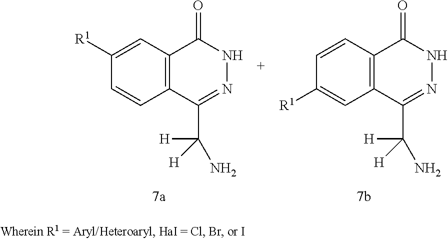

Compounds of Formula (I) wherein R¹ is aryl or heteroaryl, may be prepared according to General Reaction Scheme I. Compounds 7a and 7b are both examples of Formula (I) wherein R¹ is aryl or heteroaryl and R^{3a} and R^{3b} are H. A haloaryl cyclic anhydride 1 is treated with bis(nucleophile) such as hydrazine hydrate in acetic acid at elevated temperature, to form a phthalhydrazide 2 which is treated with a halogenating agent, for example POCl₃ to afford the trihalophthalazine 3. Treatment of the trihalophthalazine 3 with an alcohol, for example benzyl alcohol and NaH in THF at 0° C., furnishes dihaloalkoxyphthalazine 4a and 4b as a mixture of regioisomers. The mixture of 4a and 4b is subjected to palladium catalyzed cross coupling conditions, such as the Stille coupling or the Suzuki coupling with aryl/heteroaryl metal reactants, for example with the corresponding aryl/heteroaryl-tributyltin or aryl/heteroaryl boronic acids/esters to provide substituted haloalkoxyphthalazine 5a and 5b as a mixture of regioisomers. The substituted haloalkoxyphthalazine mixture 5a and 5b is subjected to metal-mediated cyanation conditions with for example, Pd₂(dba)₃, dppf, Zn and ZnCN₂ in DMF at elevated temperature and the resulting cyanoalkoxyphthalazine mixture 6a and 6b is subjected to hydrogenation conditions, for example with Pd/C, HCl and H₂ in methanol to give the phthalazinone methylamine mixture 7a and 7b. The regioisomeric mixture of 7a and 7b is separated by chromatography, such as supercritical fluid chromatography (SFC) to furnish the desired compounds 7a and 7b of Formula (I).

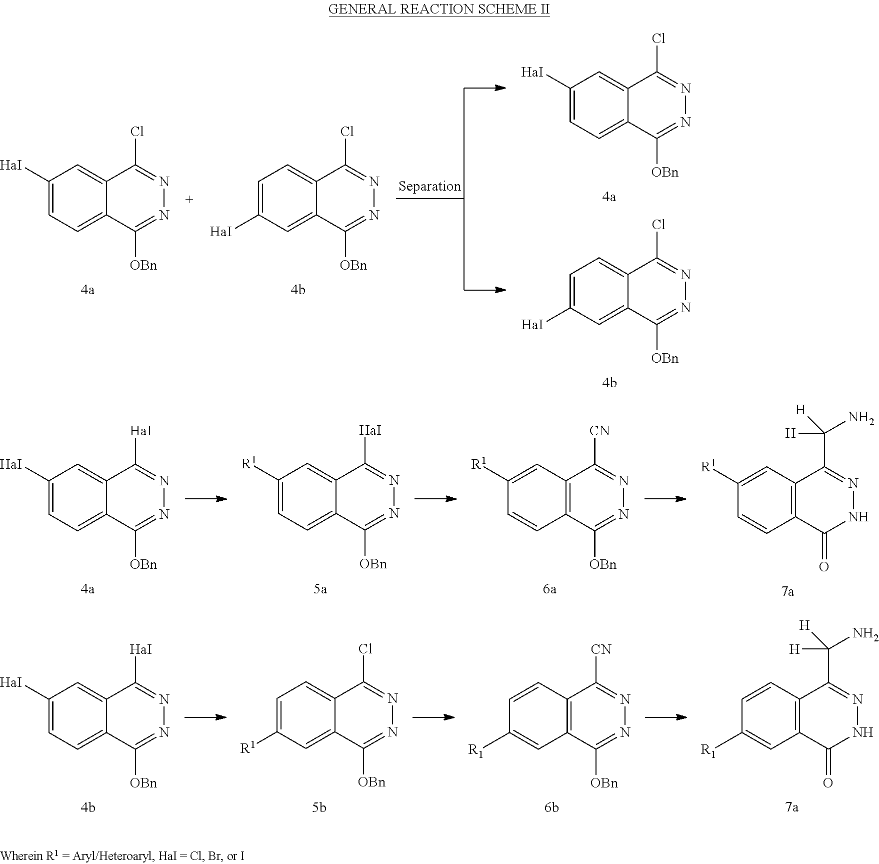

Compounds of Formula (I) wherein R¹ is aryl or heteroaryl, may be prepared according R Scheme II. Compounds 7a and 7b are both examples of Formula (I) wherein RH is aryl or heteroaryl and R^{3a} and R^{3b} are H. The mixture of regioisomers 4a and 4b are separated by chromatography, such as supercritical fluid chromatography (SFC) giving isomerically pure dihaloalkoxyphthalazines 4a and 4b. 4a or 4b is then subjected to metal-mediated cross coupling conditions, for example Suzuki conditions, with aryl/heteroaryl boronic acids/esters to provide the substituted haloalkoxyphthalazine 5a or 5b. The substituted haloalkoxyphthalazine 5a or 5b is subjected to metal-mediated cyanation conditions with for example, Pd₂(dba)₃, dppf, Zn and ZnCN₂ in DMF at elevated temperature to furnish cyanoalkoxyphthalazine 6a or 6b. Cyanoalkoxyphthalazine 6a or 6b is subjected to hydrogenation conditions, for example with Pd/C, HCl and H₂ in methanol to give the phthalazinone 7a or 7b to furnish the desired compounds of Formula (I).

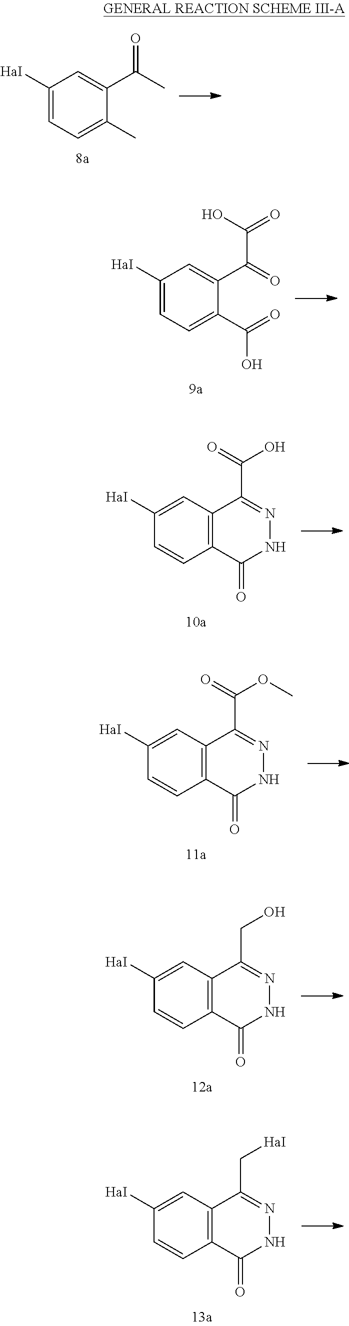

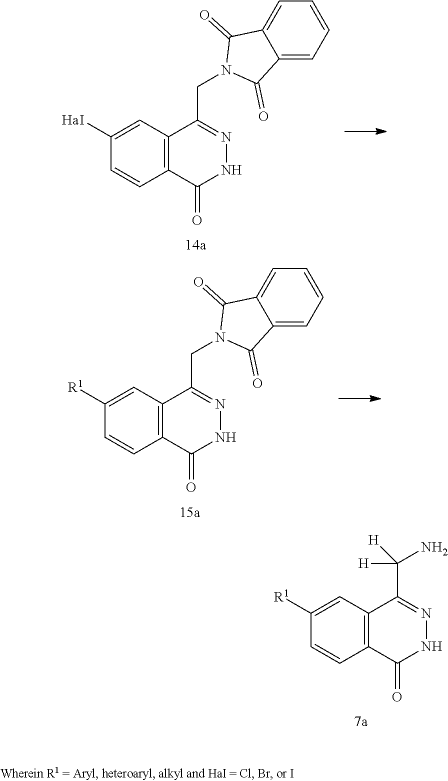

Compounds of Formula (I) wherein R¹ is aryl, heteroaryl, heterocyclyl or alkyl, may be prepared according to General Reaction Scheme III-A. Compound 7a is an example of Formula (I) wherein R¹ is aryl, heteroaryl, heterocyclyl or alkyl and R^{3a} and R^{3b} are H. 1-(5-halo-2-methylphenyl)ethenone 8a is treated with an oxidant, for example KMnO₄ in water at 50° C. to furnish 2-(carboxycarbonyl)-4-halobenzoic acid 9a. Condensation of 9a, for example with hydrazine hydrate in ethanol at elevated temperature, yields 7-halo-4-oxo-3,4-dihydrophthalazine-1-carboxylic acid 10a which is then esterified with acid and alcohol, for example sulfuric acid and methanol. Methyl 7-halo-4-oxo-3,4-dihydrophthalazine-1-carboxylate 11a is reduced via hydride reduction, for example with sodium borohydride and CaCl₂ in methanol, to afford the 6-halo-4-(hydroxymethyl)phthalazin-1 (2H)-one 12a, which is then treated with halogenating agent, for example thionyl chloride for 12 hours to provide 6-halo-4-(halomethyl)phthalazin-1 (2H)-one 13a. Nucleophilic S_{N}2 displacement of 13a with a nitrogen nucleophile for example, potassium phthalimide in DMF at elevated temperature furnishes 14a which is subjected to metal-mediated cross coupling conditions, for example Suzuki conditions, with aryl/heteroaryl/heterocyclyl/alkyl boronic acids/esters to provide phthalazinone coupling product 15a. The phthalimide protecting group of 15a is removed under solvolysis conditions, for example with hydrazine hydrate in ethanol to furnish the desired compound 7a of Formula (I).

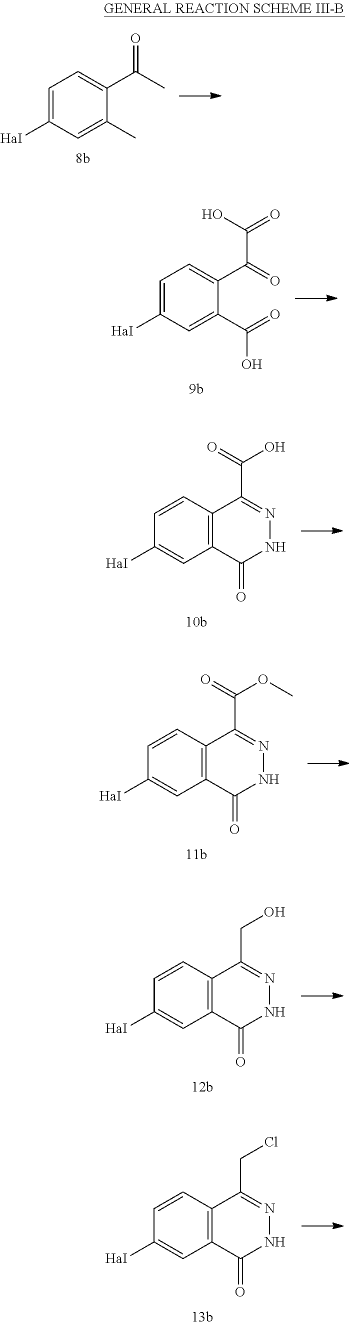

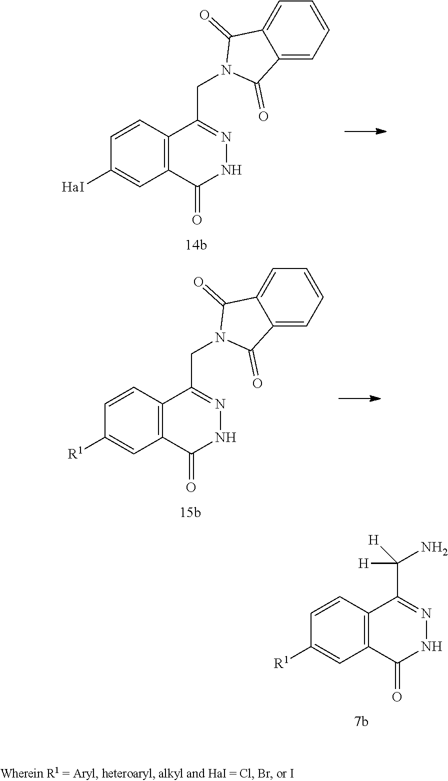

Compounds of Formula (I) wherein R¹ is aryl, heteroaryl, heterocyclyl or alkyl, may be prepared according to General Reaction Scheme III-B. Compound 7b is an example of Formula (I) wherein R¹ is aryl, heteroaryl, heterocyclyl or alkyl and R^{3a} and R^{3b} are H. 1-(5-halo-2-methylphenyl)ethenone 8b is treated with an oxidant, for example KMnO₄ in water at 50° C. to furnish 2-(carboxycarbonyl)-4-halobenzoic acid 9b. Condensation of 9b, for example with hydrazine hydrate in ethanol at elevated temperature, yields 7-halo-4-oxo-3,4-dihydrophthalazine-1-carboxylic acid 10b which is then esterified with acid and alcohol, for example sulfuric acid and methanol. Methyl 7-halo-4-oxo-3,4-dihydrophthalazine-1-carboxylate 11b is reduced via hydride reduction, for example with sodium borohydride and CaCl₂ in methanol, to afford the 6-halo-4-(hydroxymethyl)phthalazin-1 (2H)-one 12b, which is then treated with halogenating agent, for example thionyl chloride for 12 hours to provide 6-halo-4-(halomethyl)phthalazin-1 (2H)-one 13b. Nucleophilic S_{N}2 displacement of 13b with a nitrogen nucleophile for example, potassium phthalimide in DMF at elevated temperature furnishes 14b which is subjected to metal-mediated cross coupling conditions, for example Suzuki conditions, with aryl/heteroaryl/heterocyclyl/alkyl boronic acids/esters to provide phthalazinone coupling product 15b. The phthalimide protecting group of 15b is removed under solvolysis conditions, for example with hydrazine hydrate in ethanol to furnish the desired compound 7b of Formula (I).

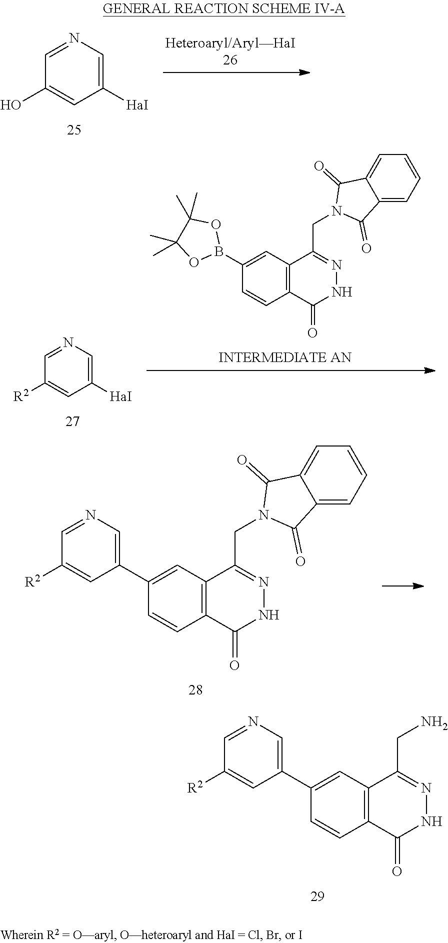

Compounds of Formula (I) wherein R¹ is pyridyl and R² is —O-aryl or —O-heteroaryl, may be prepared according to General Reaction Scheme IV-A. Compound 29 is an example of Formula (I) wherein R¹ is pyridyl, R² is —O-aryl or —O-heteroaryl, and R^{3a} and R^{3b} are H. 5-bromopyridin-3-ol 25 is heated with an appropriately substituted aryl/heteroaryl halide 26, for example in a mixture of DMF and NaH, to furnish 3-halo-5-R²-oxypyridine 27. 3-halo-5-R²-pyridine 27 is coupled to boronic acid, Intermediate AN under palladium catalyzed cross coupling conditions, for example the Suzuki coupling, to generate R²-pyridyl coupling product 28. R²-pyridyl coupling product 28 is subjected to solvolysis conditions, for example with hydrazine hydrate in ethanol, to furnish the free amine 29 of Formula (I).

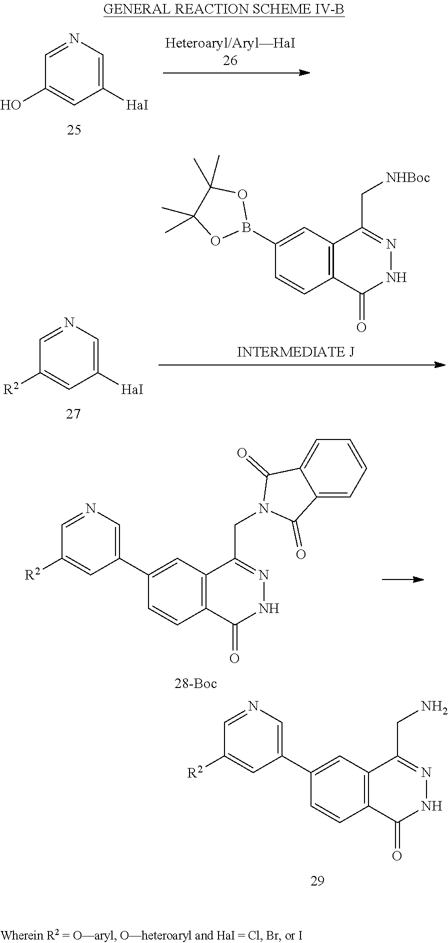

Compounds of Formula (I) wherein R¹ is pyridyl and R² is —O-aryl or —O-heteroaryl, may be prepared according to General Reaction Scheme IV-B. Compound 29 is an example of Formula (I) wherein R¹ is pyridyl, R² is —O-aryl or —O-heteroaryl, and R^{3a} and R^{3b} are H. 5-bromopyridin-3-ol 25 is heated with aryl/heteroaryl halide 26, for example in a mixture of DMF and NaH, to furnish 3-bromo-5-R²-pyridine 27. 3-bromo-5-R²-pyridine 27 is coupled to boronic acid Intermediate J under palladium catalyzed cross coupling conditions, for example Suzuki conditions, to generate coupling product 28-Boc. Coupling product 28-Boc is subjected to acidic conditions, for example with TFA, to furnish the desired compound 29 of Formula (I).

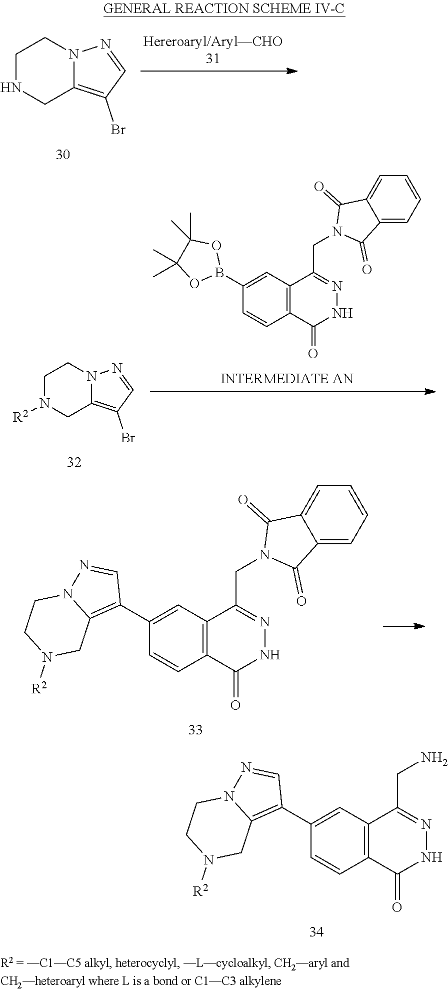

Compounds of Formula (I) wherein R¹ is 5-R²-6,7-dihydro-4H-pyrazolo[1,5-a]pyrazine-3-yl and R² is —C1-C5 alkyl, heterocyclyl, -L-cycloalkyl, —CH₂-aryl and —CH₂-heteroaryl where L is a bond or C1-C3 alkylene, may be prepared according to General Reaction Scheme IV-C. Compound 34 is an example of Formula (I) wherein R¹ is 5-R²-6,7-dihydro-4H-pyrazolo[1,5-a]pyrazine-3-yl, R² is —C1-C5 alkyl, heterocyclyl, -L-cycloalkyl, —CH₂-aryl and —CH₂-heteroaryl where L is a bond or C1-C3 alkylene and R^{3a} and R^{3b} are H. 3-Bromo-4,5,6,7-tetrahydropyrazolo[1,5-a]pyrazine 30 is reacted with aldehyde or ketone 31 under reductive amination conditions, for example with sodium borohydride in methanol, to form R²-substituted product 32. Amination product 32 is coupled to boronic ester Intermediate AN under palladium catalyzed cross-coupling, for example Suzuki conditions, furnishing coupling product 33. Coupling product 33 is then exposed to solvolysis conditions, for example with hydrazine hydrate, to deliver free amine 34 of Formula (I).

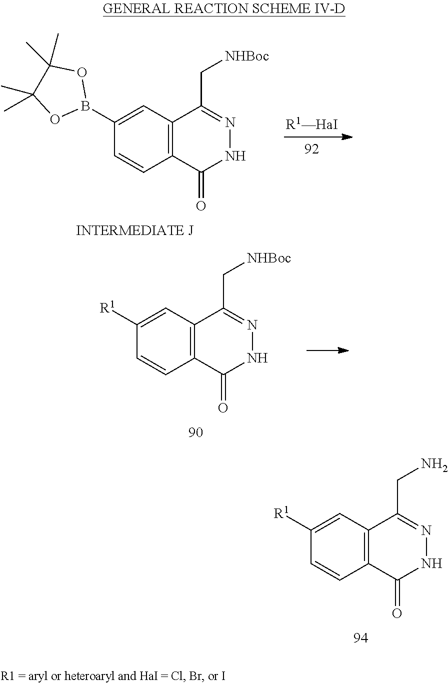

Compounds of Formula (I) wherein R¹ is aryl or heteroaryl, may be prepared according to General Reaction Scheme IV-D. Compound 94 is an example of Formula (I) wherein R¹ is an appropriately substituted aryl or heteroaryl and R^{3a} and R^{3b} are H. N-Boc boronic ester Intermediate J is coupled to an aryl/heteroaryl-substituted halide 92 under palladium catalyzed cross coupling conditions, for example Suzuki coupling conditions, to generate N-Boc-R¹-substituted coupling product 90. N-Boc-R¹-substituted coupling product 90 is subjected to acidic conditions to remove the Boc group, for example TFA, to afford R¹-substituted amine 94 of Formula (I).

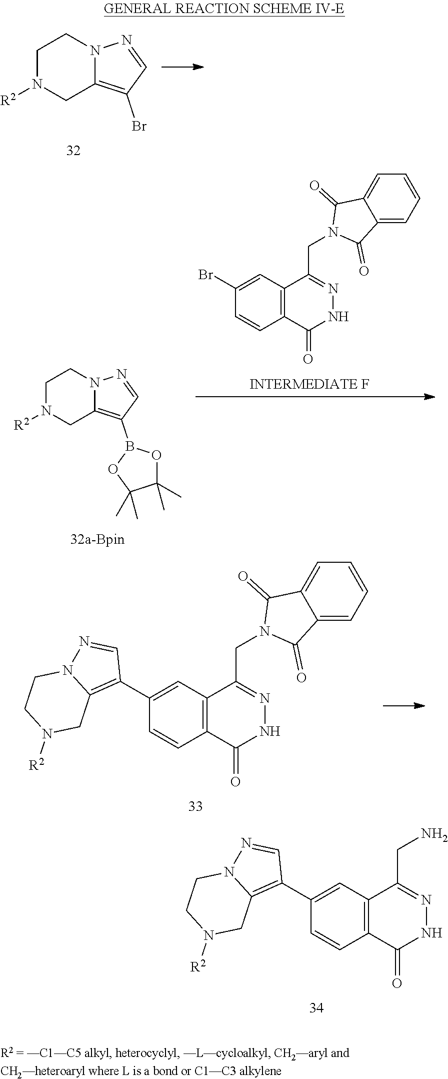

Compounds of Formula (I) wherein R¹ is 5-R²-6,7-dihydro-4H-pyrazolo[1,5-a]pyrazine-3-yl and R² is —C1-C5 alkyl, heterocyclyl, -L-cycloalkyl, —CH₂-aryl and —CH₂-heteroaryl where L is a bond or C1-C3 alkylene, may be prepared according to General Reaction Scheme IV-E. Compound 34 is an example of Formula (I) wherein R¹ is 5-R²-6,7-dihydro-4H-pyrazolo[1,5-a]pyrazine-3-yl, R² is —C1-C5 alkyl, heterocyclyl, -L-cycloalkyl, —CH₂-aryl and —CH₂-heteroaryl where L is a bond or C1-C3 alkylene and R^{3a} and R^{3b} are H. 3-Bromo-5-R²-6,7-dihydro-4H-pyrazolo[1,5-a]pyrazine 32 is borylated, for example with Miyaura conditions, to supply boronate ester 32a-Bpin. Borylation product 32a-Bpin is coupled to Intermediate F under palladium catalyzed cross-coupling conditions, for example Suzuki conditions, furnishing coupling product 33a. Coupling product 33a is deprotected under acidic conditions, for example TFA, to deliver amine 34a of Formula (I).

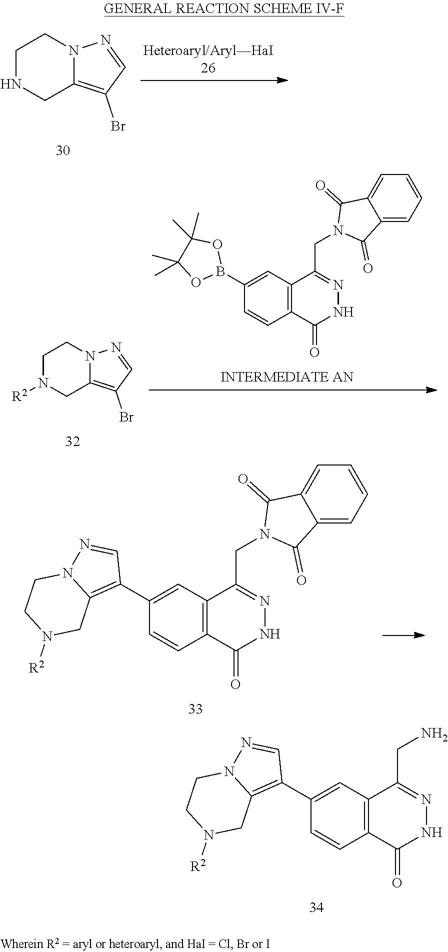

Compounds of Formula (I) wherein R¹ is 5-R²-6,7-dihydro-4H-pyrazolo[1,5-a]pyrazine-3-yl and R² is aryl or heteroaryl, may be prepared according to General Reaction Scheme IV-F. Compound 34 is an example of Formula (I) wherein R¹ is 5-R²-6,7-dihydro-4H-pyrazolo[1,5-a]pyrazine-3-yl, R² is aryl or heteroaryl and R^{3a} and R^{3b} are H. 3-Bromo-4,5,6,7-tetrahydropyrazolo[1,5-a]pyrazine 30 is reacted with aryl/heteroaryl halide 26, under copper catalyst mediated Ullman coupling conditions, for example with Cu(I)I, Cs₂CO₃, L-proline in DMF, at elevated temperature, to form amination product 32. R²-substituted amination product 32 is coupled to Intermediate AN under palladium catalyzed cross-coupling conditions, for example Suzuki conditions, furnishing coupling product 33. Coupling product 33 is subjected to solvolysis conditions, for example hydrazine hydrate, to deliver amine 34 of Formula (I).

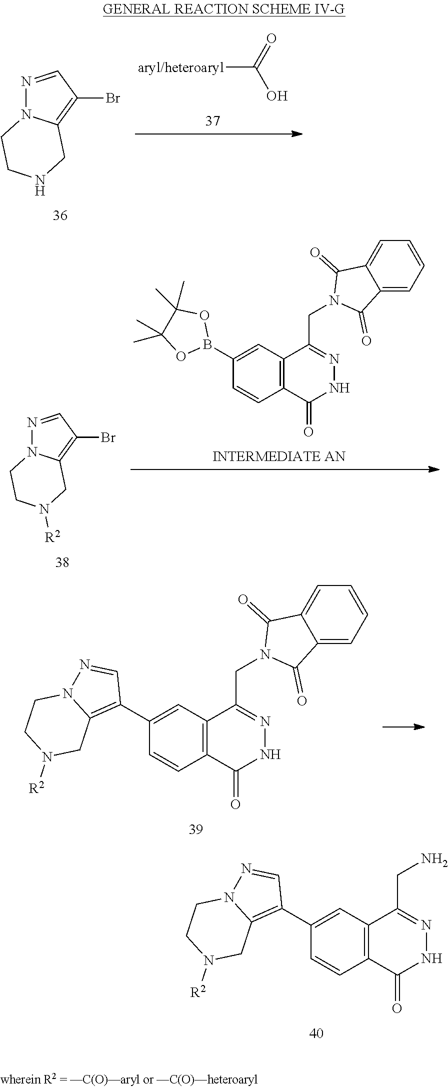

Compounds of Formula (I) wherein R¹ is 5-R²-6,7-dihydro-4H-pyrazolo[1,5-a]pyrazine-3-yl and R² is —C(O)— aryl or —C(O)— heteroaryl, may be prepared according to General Reaction Scheme IV-G. Compound 40 is an example of Formula (I) wherein R¹ is 5-R²-6,7-dihydro-4H-pyrazolo[1,5-a]pyrazine-3-yl, R² is —C(O)— aryl or —C(O)— heteroaryl and R^{3a} and R^{3b} are H. 3-Bromo-4,5,6,7-tetrahydropyrazolo[1,5-a]pyrazine 30 is coupled to carboxylic acid 37 with a coupling reagent, for example 1-[bis(dimethylamino)methylene]-1H-1,2,3-triazolo[4,5-b]pyridinium 3-oxid hexafluorophosphate (HATU) with a base such as triethylamine in DMF to form amide 38. Amide 38 is coupled to boronic ester Intermediate AN, under palladium catalyzed cross-coupling conditions, for example the Suzuki coupling, to yield coupling product 39. R²-coupling product 39 is subjected to solvolysis conditions, for example with hydrazine monohydrate, to remove the phthalimide moiety and provide amine compound 40 of Formula (I).

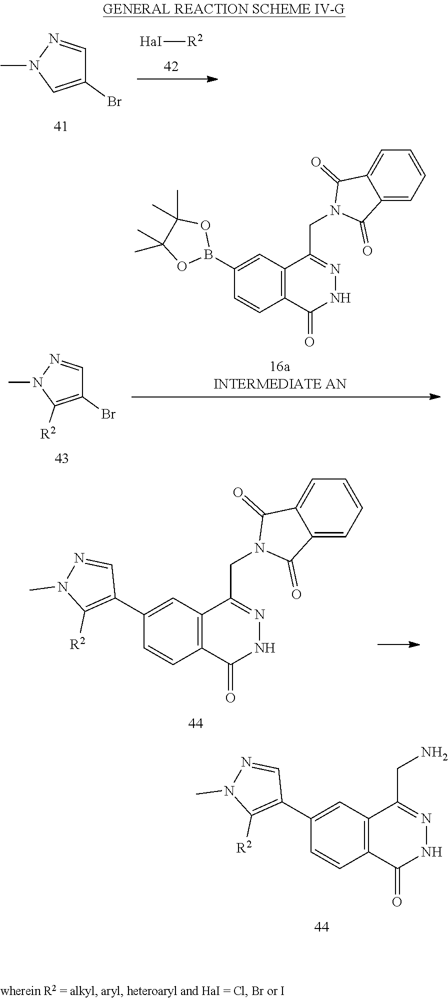

Compounds of Formula (I) wherein R¹ is 1-methyl-5-R²-1H-pyrazole-4-yl and R² is alkyl, aryl or heteroaryl, may be prepared according to General Reaction Scheme IV-H. Compound 45 is an example of Formula (I) wherein R¹ is 1-methyl-5-R²-1H-pyrazole-4-yl, R² is alkyl, aryl or heteroaryl and R^{3a} and R^{3b} are H. 4-bromo-1-methyl-1H-pyrazole 41 is coupled to alkyl/aryl/heteroaryl-substituted halide 42, for example with palladium acetate, DavePhos, tetrabutylammoniumacetate, pivalic acid in NMP at elevated temperature to furnish R²-substituted-bromopyrazole 43. R²-substituted-bromopyrazole 43 is coupled to Intermediate AN under palladium-mediated cross coupling conditions, for example Suzuki conditions, to provide R²-substituted coupling product 44. The coupling product 44 is subjected to solvolysis conditions, for example with hydrazine hydrate to furnish amine 45 of Formula (I).

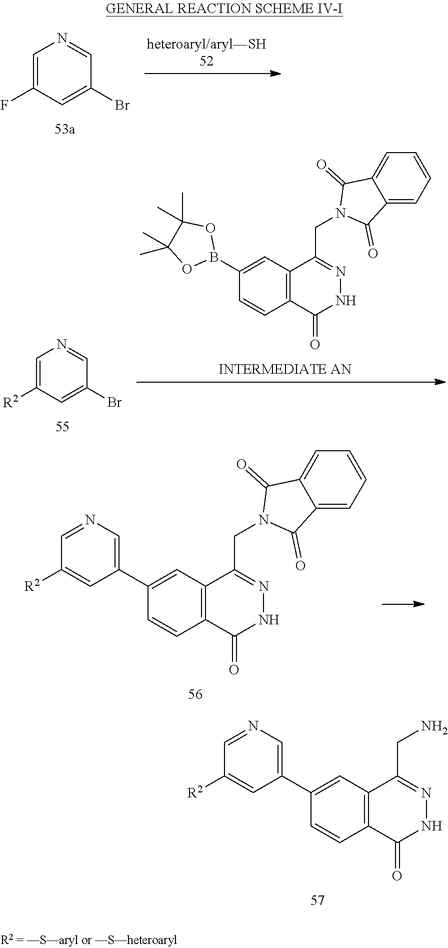

Compounds of Formula (I) wherein R¹ is pyridyl, R² is —S-aryl or —S-heteroaryl, may be prepared according to General Reaction Scheme IV-I. Compound 57 is an example of Formula (I) wherein R¹ is pyridyl, R² is —S-aryl or —S-heteroaryl and R^{3a} and R^{3b} are H. 3-bromo-5-fluoropyridine 53a is subjected to S_{N}Ar substitution conditions, for example sodium aryl/heteroaryl thiolate 54, NaH in DMF at elevated temperature, to provide 3-bromo-5-(aryl/heteroarylthio)pyridine 55. 3-bromo-5-(aryl/heteroarylthio)pyridine 55 is coupled with boronic ester Intermediate AN under palladium cross coupling conditions, for example Suzuki conditions, to furnish R²-pyridyl-cross coupling product 56. R²-pyridyl-cross coupling product 56 is subjected to solvolysis conditions, with for example hydrazine hydrate, to produce amine 57 of Formula (I).

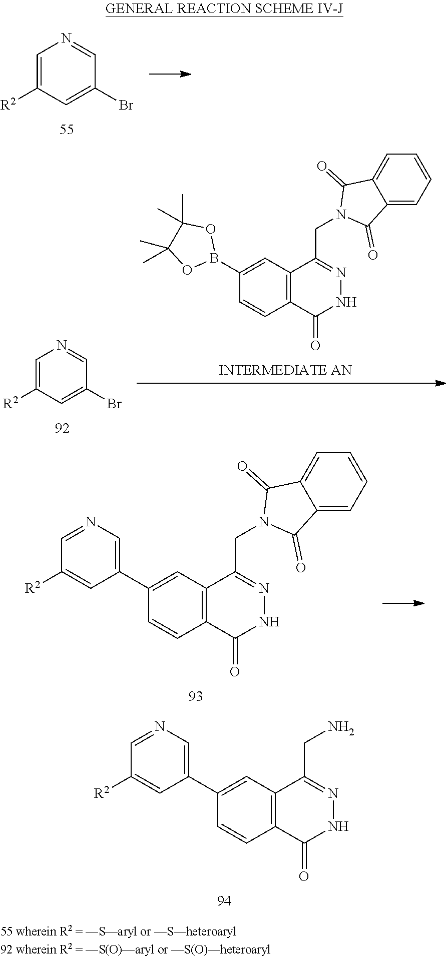

Compounds of Formula (I) wherein R¹ is pyridyl, R² is —S(O)-aryl or —S(O)-heteroaryl, may be prepared according to General Reaction Scheme IV-J. Compound 94 is an example of Formula (I) wherein R¹ is pyridyl, R² is —S(O)-aryl or —S(O)-heteroaryl and R^{3a} and R^{3b} are H. 3-bromo-5-(R²-thio)pyridine 55 is subjected to oxidation conditions, for example mCPBA in dichloromethane at ambient temperature, to afford 3-bromo-5-(R²-sulfinyl)pyridine 92. 3-bromo-5-(R²-sulfinyl)pyridine 92 is coupled with boronic ester intermediate AN under palladium catalyzed cross-coupling conditions, for example Suzuki conditions, to provide R²-substituted sulfinylpyridyl product 93. R²-substituted sulfinylpyridyl product 93 is subjected to solvolysis conditions, for example with hydrazine hydrate, to procure the R²-substituted sulfinylpyridyl amine 94 of Formula (I).

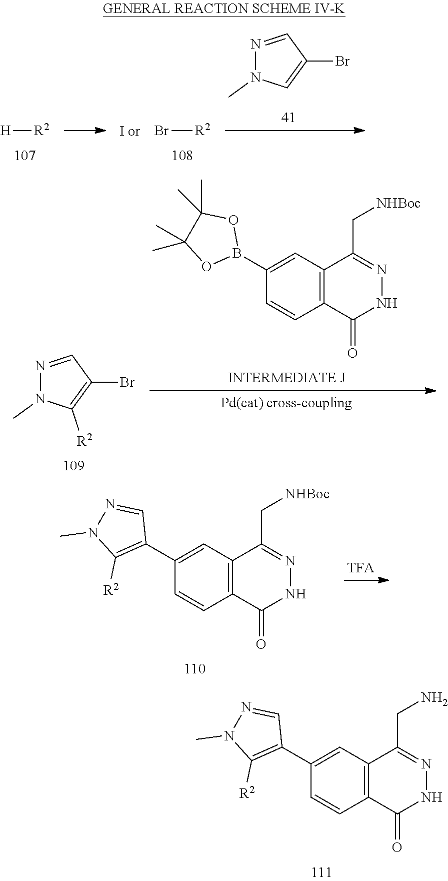

Compounds of Formula (I) wherein R¹ is 1-methyl-5-R²-1H-pyrazole-4-yl and R² is alkyl, aryl or heteroaryl, may be prepared according to General Reaction Scheme IV-K. Compound 111 is an example of Formula (I) wherein R¹ is 1-methyl-5-R²-1H-pyrazole-4-yl, R² is alkyl, aryl or heteroaryl and R^{3a} and R^{3b} are H. H—R² 107 is halogenated, for example with a halogenating agent such N-bromosuccinimide or N-chlorosuccinimide under palladium catalyzed conditions such as palladium acetate in the presence of an acid such as p-toluenesulfonic acid in a solvent such as dichloroethane under elevated temperature for example 70° C. to give halide 108. 4-bromo-1-methyl-1H-pyrazole 41 is coupled to alkyl/aryl/heteroaryl-substituted halide 108, for example with palladium acetate, DavePhos, tetrabutylammoniumacetate, pivalic acid in NMP at elevated temperature to furnish R²-substituted-bromopyrazole 109. R²-substituted-bromopyrazole 109 is coupled to Intermediate J under palladium-mediated cross coupling conditions, for example Suzuki conditions, to provide N-Boc-R²-substituted coupling product 110. The coupling product 110 is subjected to acidic conditions to remove the Boc group, for example TFA, to afford R²-substituted amine 111 of Formula (I).

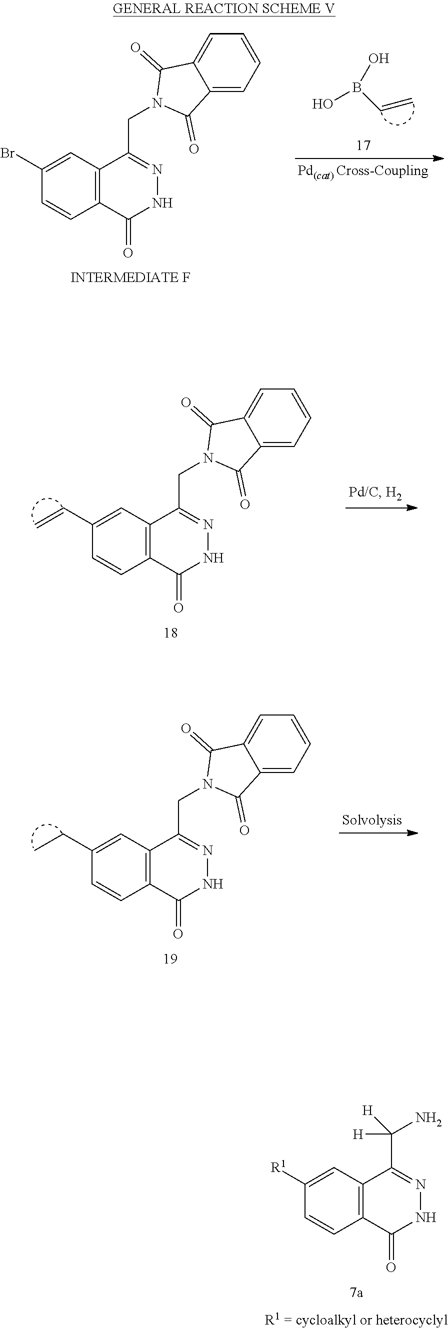

Compounds of Formula (I) where in R¹ is cycloalkyl or heterocyclyl, may be prepared according to General Reaction Scheme V. Compound 7a is an example of Formula (I) wherein R¹ is a cycloalkyl or heterocyclyl and R^{3a} and R^{3b} are H.2-((7-bromo-4-oxo-3,4-dihydrophthalazin-1-yl)methyl)isoindoline-1,3-dione Intermediate F is coupled to a 4-6 membered ring olefin boronic acid 17 under palladium catalyzed coupling conditions, for example Suzuki coupling conditions to provide the appropriate olefinic coupling product 18. The olefin coupling product 18 is then subjected to hydrogenation conditions, for example Pd/C and H₂, to furnish the appropriate hydrogenation product 19. The hydrogenation product is then subjected to hydrazine solvolysis conditions, for example with hydrazine hydrate to provide the primary amine compound 7a of formula (I).

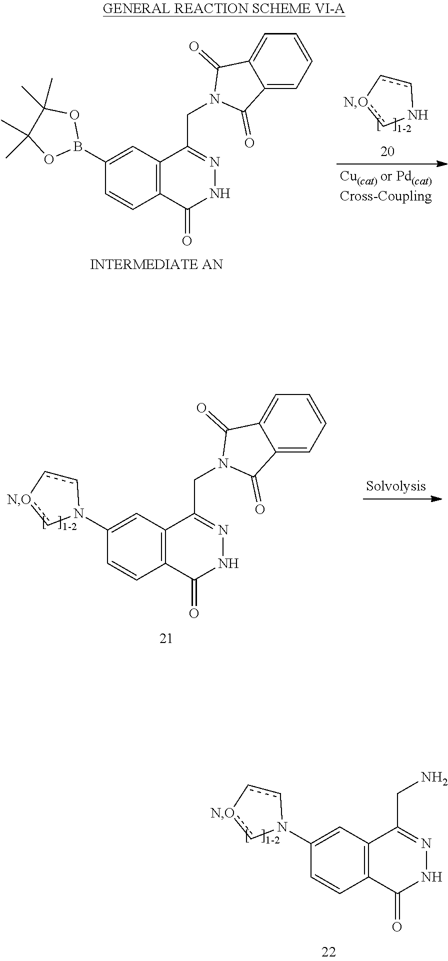

Compounds of Formula (I) wherein R¹ is N-linked heteroaryl or N-linked heterocyclyl, may be prepared according to General Reaction Scheme VI-A. Compound 22 is an example of Formula (I) wherein R¹ is N-linked heteroaryl or N-linked heterocyclyl and R^{3a} and R^{3b} are H. Boronic ester intermediate AN, is subjected to metal catalyzed cross-coupling conditions, for example Ullman, Buchwald-Hartwig or Chan-Lam conditions with a nitrogen containing heterocyclyl or a nitrogen containing heteroaryl 20 to provide the appropriate N-coupled product 21. This N-coupled product 21 is subjected to solvolysis conditions, for example with hydrazine hydrate to remove the phthalimide to furnish the desired primary amine 22 of Formula (I).

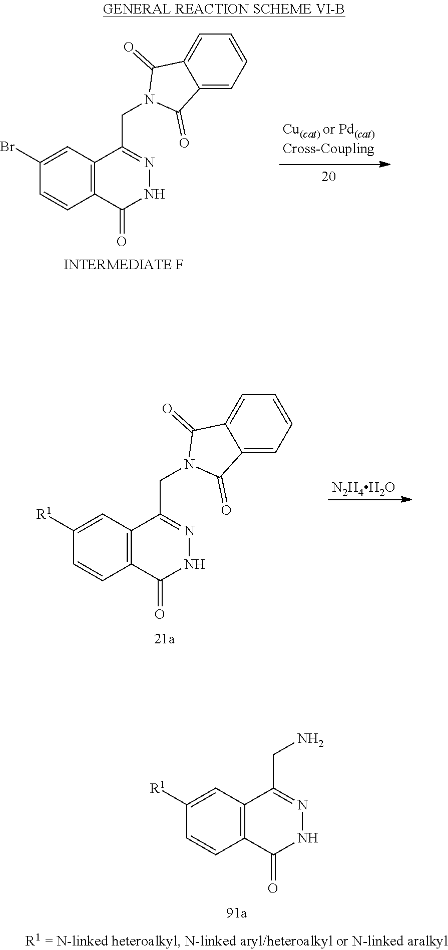

Compounds of Formula (I) wherein R¹ is N-linked heteroaryl or N-linked heterocyclyl, may be prepared according to General Reaction Scheme VI-B. Compound 23 is an example of Formula (I) wherein R¹ is N-linked heteroaryl or N-linked heterocyclyl and R^{3a} and R^{3b} are H. 2-((7-bromo-4-oxo-3,4-dihydrophthalazin-1-yl)methyl)isoindoline-1,3-dione Intermediate F, is subjected to metal catalyzed cross-coupling conditions, for example Ullman, Buchwald-Hartwig or Chan-Lam conditions with a nitrogen containing heterocyclyl or nitrogen containing heteroaryl 20 to provide the appropriate N-coupled product 21a. This N-coupled product 21a is then subjected to solvolysis conditions, for example with hydrazine hydrate to furnish the desired primary amine 91a of Formula (I).

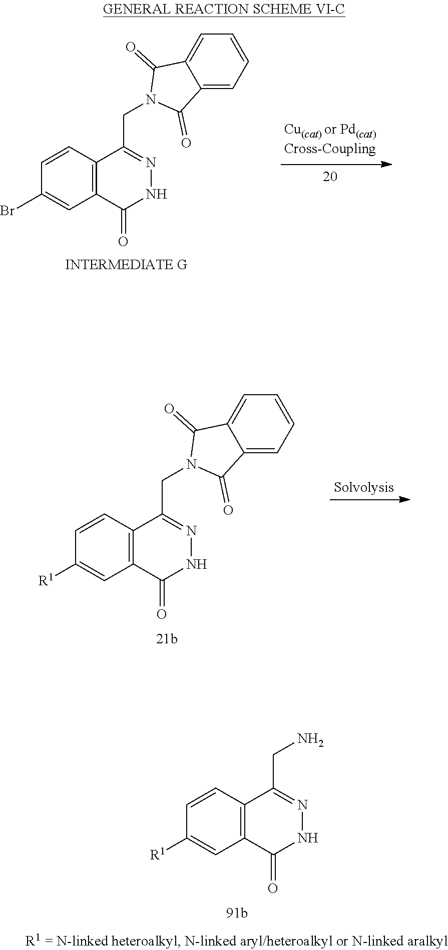

Compounds of Formula (I) wherein R¹ is N-linked heteroalkyl, N-linked arylheteroalkyl or N-linked aralkyl, may be prepared according to General Reaction Scheme VI-C. Compound 24 is an example of Formula I wherein R¹ is N-linked heteroalkyl, N-linked arylheteroalkyl or N-linked aralkyl and R^{3a} and R^{3b} are H. 2-((6-bromo-4-oxo-3,4-dihydrophthalazin-1-yl)methyl)isoindoline-1,3-dione intermediate F, is subject to metal catalyzed cross-coupling conditions, for example Ullman, Buchwald-Hartwig or Chan-Lam conditions with an heteroalkyl/arylheteroalkyl/aralkyl amine 20 to provide the appropriate N-coupled product 21b. This N-coupled product 21b is then subject to solvolysis conditions, for example with hydrazine hydrate to furnish the desired R¹ substituted product 91b of Formula (I).

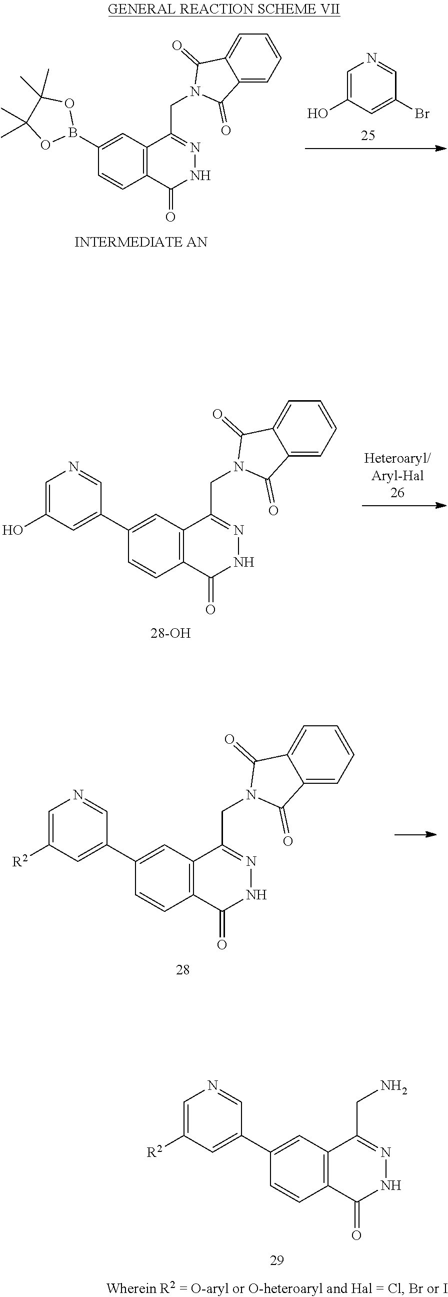

Compounds of Formula (I) wherein R¹ is pyridyl and R² is O-aryl or O-heteroaryl, may be prepared according to General Reaction Scheme VII. Compound 29 is an example of Formula (I) wherein R¹ is pyridyl, R² is O-aryl or O-heteroaryl and R^{3a} and R^{3b} are H. 3-bromo-5-hydroxypyridine 25 is coupled to boronic ester Intermediate AN under palladium catalyzed cross coupling conditions, for example the Suzuki coupling conditions Pd(dppf)Cl₂, NaHCO₃, dioxane/water at 80° C., to generate coupling product 28-OH. Coupling product 28-OH was subjected to S_{N}Ar reaction conditions for example K₂CO₃ in DMF at 110° C. with R²-substituted aryl/heteroaryl halide 26, to furnish R²-substituted aryl/heteroaryl pyridyl ether 28. R²-substituted aryl/heteroaryl pyridyl ether 28 was subjected to solvolysis conditions, for example hydrazine hydrate to furnish free amine 29 of Formula (I).

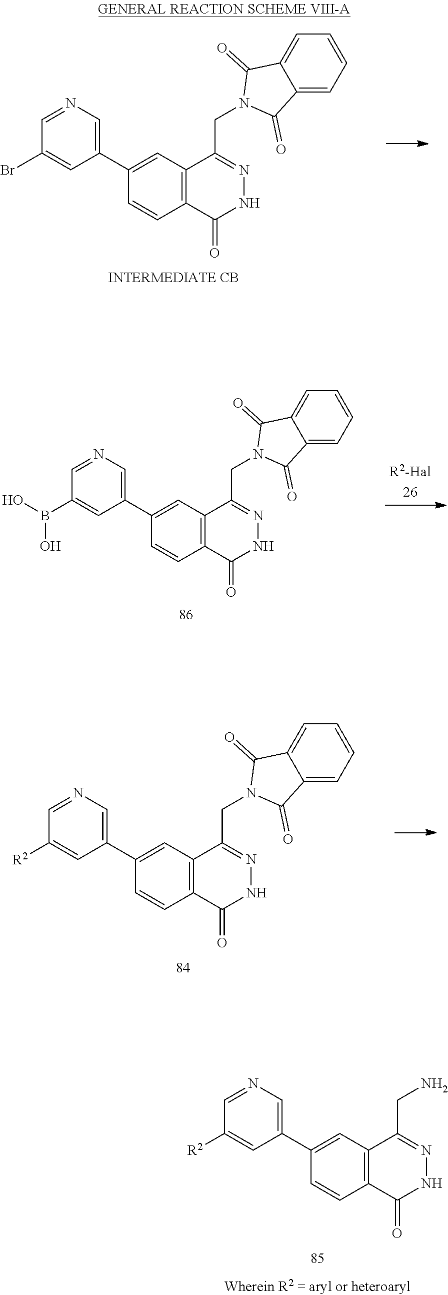

Compounds of Formula (I) wherein R¹ is pyridyl and R² is aryl or heteroaryl, may be prepared according to General Reaction Scheme VIII-A. Compound 85 is an example of Formula (I) wherein R¹ is pyridyl, R² is aryl or heteroaryl and R^{3a} and R^{3b} are H. Intermediate CB is borylated under Miyaura conditions, for example with bis(pinacolato)diboron, Pd(dppf)Cl₂, KOAc in dioxane at elevated temperature to provide boronic acid 86. Boronic acid 86 is coupled with R² halide 26 under palladium catalyzed cross coupling conditions, for example Suzuki conditions, to yield R²-pyridyl coupling product 84. R²-pyridyl coupling product 84 is subjected to solvolysis conditions, for example with hydrazine hydrate, to provide primary amine 85 of Formula (I).

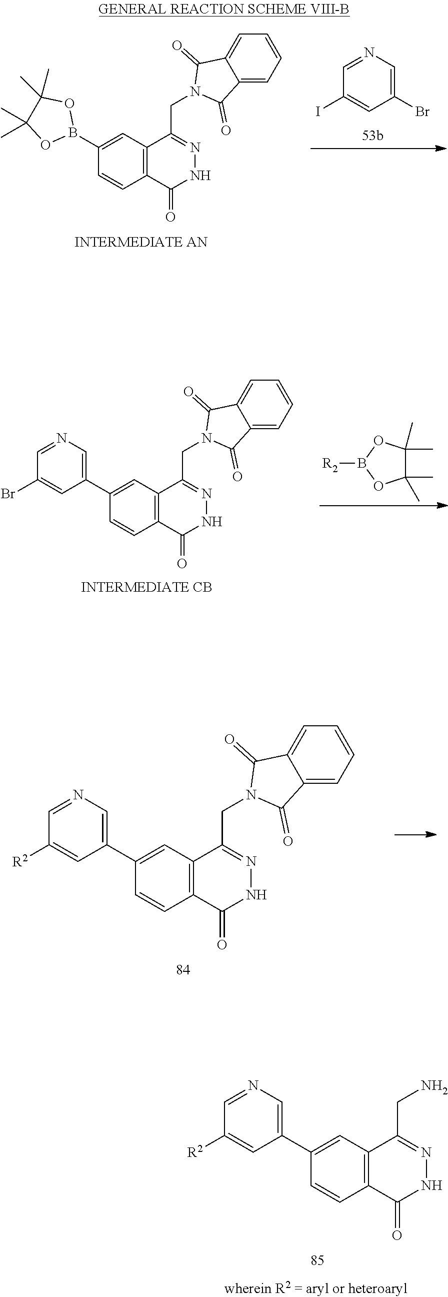

Compounds of Formula (I) wherein R¹ is pyridyl and R² is aryl or heteroaryl, may be prepared according to General Reaction Scheme VIII-B. Compound 85 is an example of Formula (I) wherein R¹ is pyridyl, R² is aryl or heteroaryl and R^{3a} and R^{3b} are H. Intermediate AN is coupled to 3-bromo-5-iodopyridine 53b under palladium catalyzed cross coupling conditions, for example Suzuki conditions, to give 3-bromo-pyridyl coupling product Intermediate CB. Intermediate CB is then coupled to aryl/heteroaryl-substituted boronic ester under palladium catalyzed cross-coupling conditions, for example Suzuki coupling conditions, to provide R²-substituted pyridyl coupling product 84. Coupling product 84 undergoes solvolysis, for example with hydrazine hydrate, to provide primary amine 85 of Formula (I).

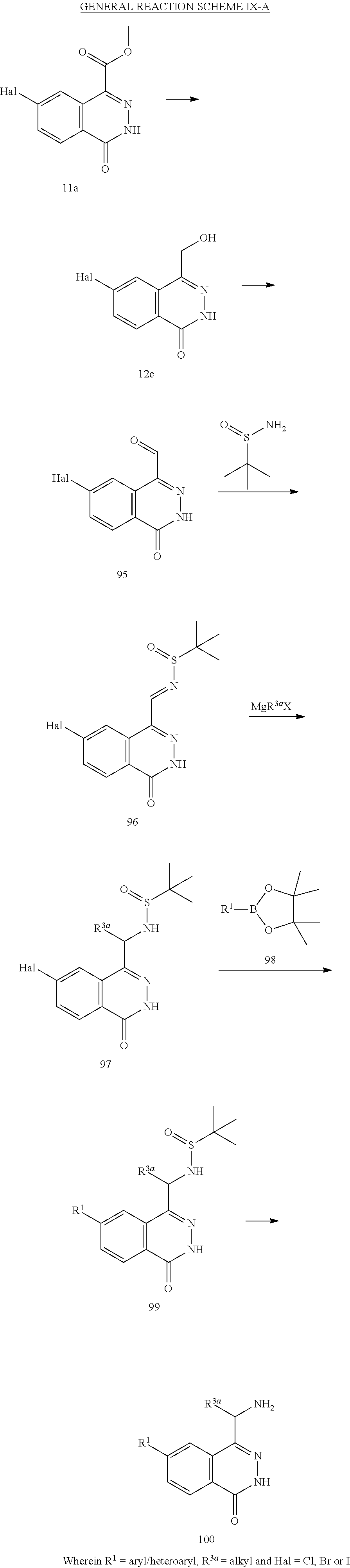

Compounds of Formula (I), wherein R¹ is aryl or heteroaryl, R^{3a} and R^{3b} are H, may be prepared according to General Reaction Scheme IX-A. Compound 100 is an example of Formula (I) wherein R¹ is aryl or heteroaryl, R^{3a} and R^{3b} are H. Methyl 7-halo-4-oxo-3,4-dihydrophthalazine-1-carboxylate 11a is reduced using a hydride reducing conditions, for example sodium borohydride, CaCl₂) in methanol at 0° C., to afford primary alcohol 6-halo-4-(hydroxymethyl)phthalazin-1 (2H)-one 12a. 6-halo-4-(hydroxymethyl)phthalazin-1 (2H)-one 12a is reacted with an oxidant, for example MnO₂ in dichloroethane to furnish 7-halo-4-oxo-3,4-dihydrophthalazine-1-carbaldehyde 95. 7-halo-4-oxo-3,4-dihydrophthalazine-1-carbaldehyde 95 is converted to the sulfinamide compound 96 by for example, adding t-butanesulfinamide, titanium tetra-iso-propoxide in THF and heating to 60° C. for 12 hours. t-Butyl sulfonamide 96 is then reacted with alkylmagnesium halide in THF at −78° C. to generate methyl sulfonamide 97. Methyl sulfonamide 97 is coupled to an appropriate R¹-substituted boronic ester under palladium catalyzed cross coupling conditions, for example Suzuki conditions, to supply R¹-substituted coupling product 99. R¹-substituted coupling product 99 is desulfinylated under acidic conditions, for example HCl/dioxane to furnish R¹-substituted primary amine 100 of Formula (I).

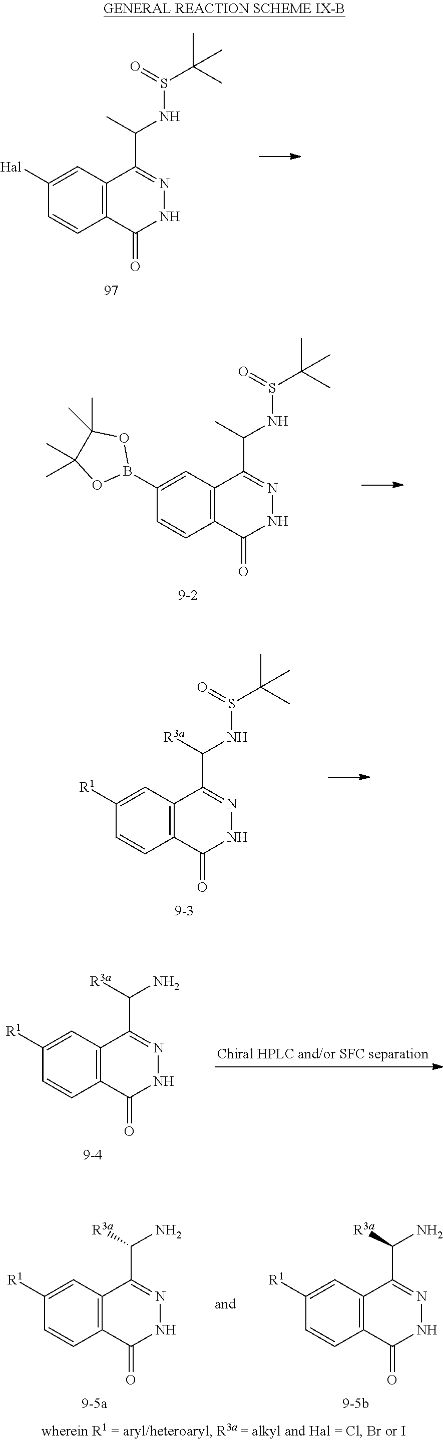

Compounds of Formula (I), wherein R¹ is aryl or heteroaryl, R^{3a} and R^{3b} are H, may be prepared according to General Reaction Scheme IX-B. Compounds 9-5a and 9-5b are examples of Formula (I) wherein R¹ is aryl or heteroaryl, R^{3a} and R^{3b} are H. t-Butyl sulfinamide intermediate 97 is borylated under Miyaura conditions, for example with bis(pinacolato)diboron, Pd(dppf)Cl₂, KOAc in dioxane at elevated temperature to provide boronic ester 9-2 which is then reacted coupled to an appropriate R¹-substituted halide under palladium catalyzed cross coupling conditions, for example Suzuki conditions, to supply R¹-substituted coupling product 9-3. R¹-substituted coupling product 9-3 is desulfinylated under acidic conditions, for example HCl/dioxane to furnish R¹-substituted primary amine 9-4 of Formula (I). The racemic mixture of 9-4 is then separated into the corresponding pure enantiomers via chiral prep HPLC and or chiral SFC to give chiral amines 9-5a and 9-5b examples of compounds of Formula (I).

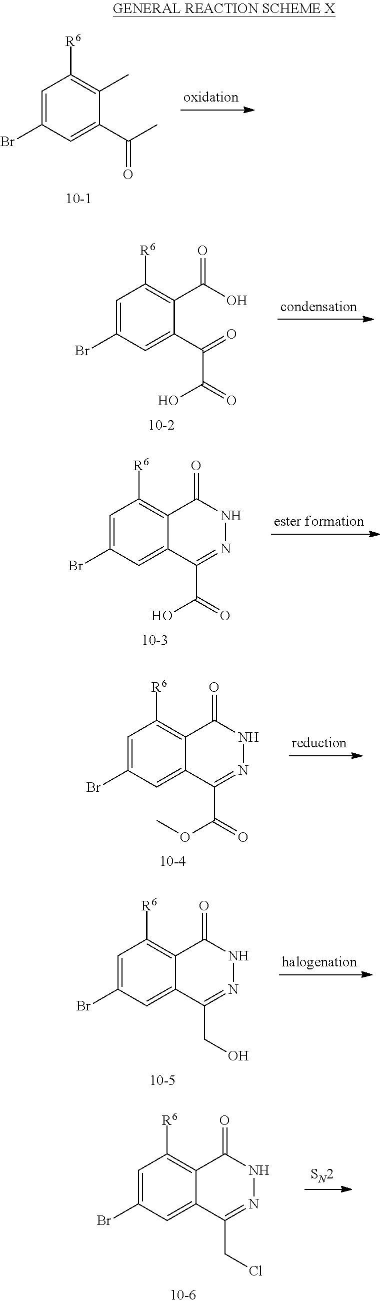

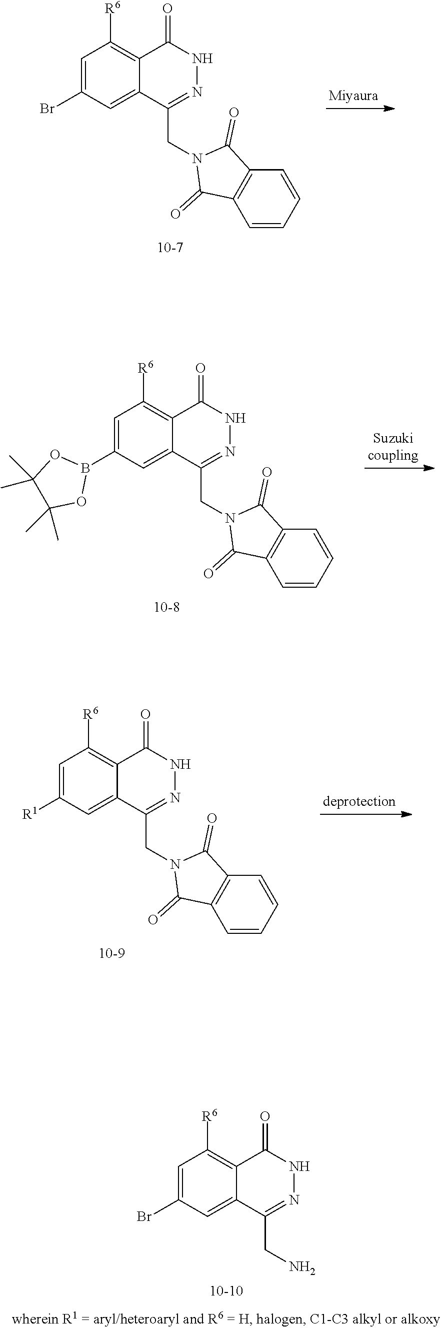

Compounds of Formula (I) wherein R¹ is aryl, heteroaryl, heterocyclyl or alkyl, may be prepared according to General Reaction Scheme X. Compound 10-10 is an example of Formula (I) wherein R¹ is aryl, heteroaryl, heterocyclyl or alkyl, R^{3a} and R^{3b} are H and R⁶ is hydrogen, halogen, C1-C3 alkyl or alkoxy. 1-(5-bromo-2-methyl-3-substituted phenyl)ethanone 10-1 is treated with an oxidant, for example KMnO₄ in water at 50° C. to furnish 4-bromo-2-(carboxycarbonyl)-6-substituted-benzoic acid 10-2. Condensation of 10-2, for example with hydrazine hydrate in ethanol at elevated temperature, yields 7-bromo-4-oxo-3,4-dihydrophthalazine-5-substituted-1-carboxylic acid 10-3 which is then esterified with acid and alcohol, for example sulfuric acid and methanol to give ester 10-4. Methyl 7-bromo-4-oxo-3,4-dihydrophthalazine-5-substituted-1-carboxylate 10-4 is reduced via hydride reduction, for example with sodium borohydride and CaCl₂ in methanol, to afford the 6-bromo-4-(hydroxymethyl)-8-substituted-phthalazin-1 (2H)-one 10-5, which is then treated with halogenating agent, for example thionyl chloride for 12 hours to provide 6-halo-4-(chloromethyl)-8-substituted-phthalazin-1 (2H)-one 10-6. Nucleophilic S_{N}2 displacement of 10-6 with a nitrogen nucleophile for example, potassium phthalimide in DF at elevated temperature furnishes 10-7 which isborylated, for example with Miyaura conditions, to give boronate ester 10-8. Palladium-mediated cross coupling conditions, for example Suzuki conditions, with boronic ester 10-8 and aryl/heteroaryl/heterocyclyl/alkyl halides provides phthalazinone coupling product 10-9. The phthalimide protecting group of 10-9 is removed under solvolysis conditions, for example with hydrazine hydrate in ethanol to furnish the desired compound 10-10 of Formula (I).

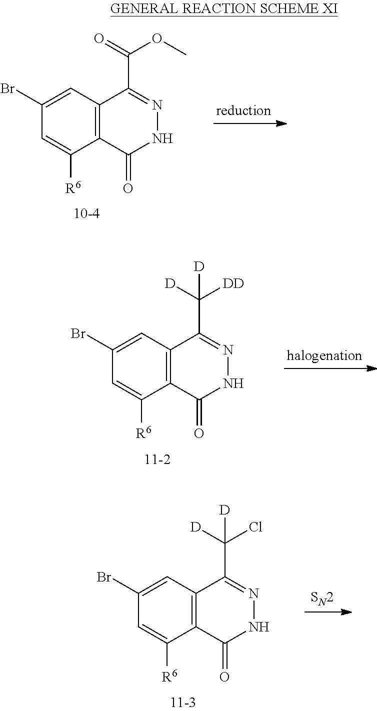

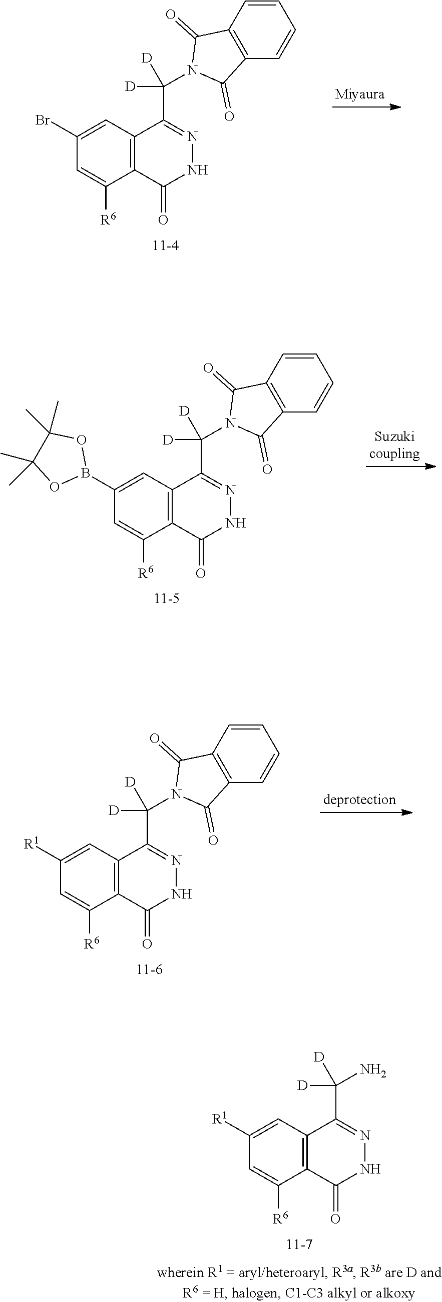

Compounds of Formula (I) wherein R¹ is aryl, heteroaryl, heterocyclyl or alkyl, may be prepared according to General Reaction Scheme XI. Compound 11-7 is an example of Formula (I) wherein R¹ is aryl, heteroaryl, heterocyclyl or alkyl, R^{3a} and R^{3b} are D and R⁶ is hydrogen, halogen, C1-C3 alkyl or alkoxy. Methyl 7-bromo-4-oxo-3,4-dihydrophthalazine-5-substituted-1-carboxylate 10-4 is reduced via deuteride reduction, for example with sodium borodeuteride and CaCl₂) in methanol-d4, to afford the 6-bromo-4-((hydroxy-d)methyl-d2)-8-substituted-phthalazin-1 (2H)-one 11-2, which is then treated with halogenating agent, for example thionyl chloride for 12 hours to provide 6-bromo-4-(chloromethyl-d2)-8-substituted-phthalazin-1 (2H)-one 11-3. Nucleophilic S_{N}2 displacement of 11-3 with a nitrogen nucleophile for example, potassium phthalimide in DMF at elevated temperature furnishes 11-4 which is borylated, for example with Miyaura conditions, to give boronate ester 11-5. Palladium-mediated cross coupling conditions, for example Suzuki conditions, with boronic ester 11-5 and aryl/heteroaryl/heterocyclyl/alkyl halides provides phthalazinone coupling product 11-6. The phthalimide protecting group of 11-6 is removed under solvolysis conditions, for example with hydrazine hydrate in ethanol to furnish the desired compound 11-7 of Formula (I).

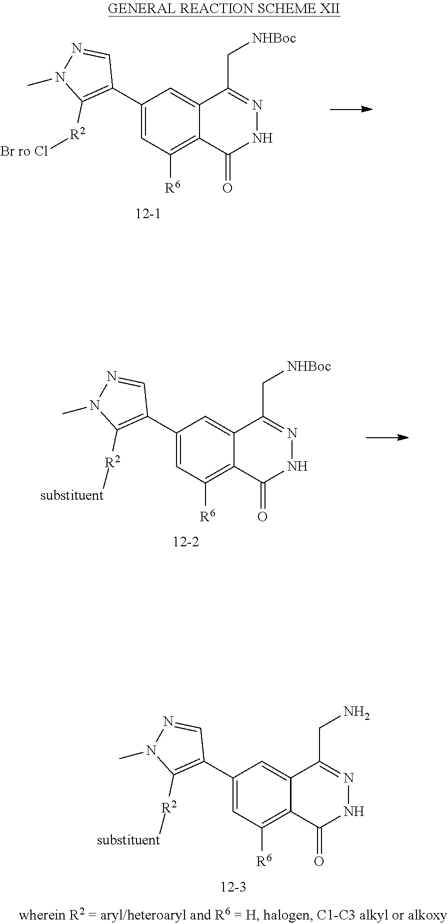

Compounds of Formula (I) wherein R² is aryl or heteroaryl, may be prepared according to General Reaction Scheme XII. Compound 12-3 is an example of Formula (I) wherein R² is aryl or heteroaryl, R^{3a} and R^{3b} are H, R⁶ is hydrogen, halogen, C1-C3 alkyl or alkoxy and substituent is alkyl, aryl or heteroaryl. Bromo or chloro compound 12-1 is subjected to palladium-mediated cross coupling conditions, for example Suzuki conditions, with an alkyl/aryl/heteroaryl boronic acid/ester to give substituent coupled product 12-2. The BOC group is then removed with acidiemc conditions, for example TFA, to afford R²-substituted amine 12-3 of Formula (I).

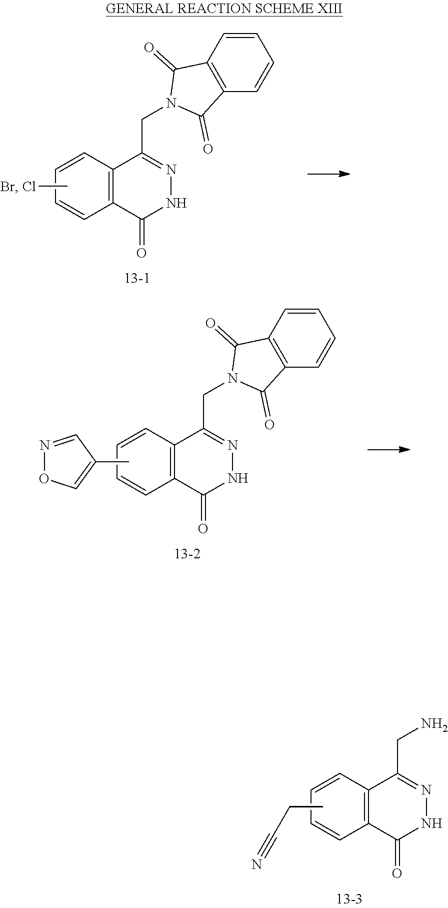

Compounds of Formula (I) wherein R¹ is alkyl-cyano, may be prepared according to General Reaction Scheme XIII. Compound 13-3 is an example of Formula (I) wherein R¹ is —CH₂CN. Bromo or chloro compound 13-1 is subjected to palladium-mediated cross coupling conditions, for example Suzuki conditions, with an isoxazole boronic acid/ester to give substituent coupled product 13-2. The isoxazole is then subjected to hydrazine hydrate in an alcoholic solvent such as ethanol at elevated temperature followed by acidic work up, for example with HCl at pH 1 to give nitrile product 13-3 of Formula (I).

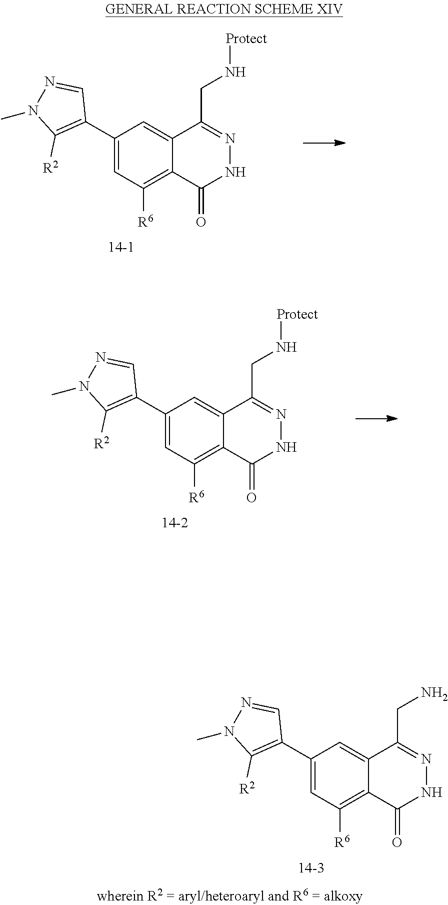

Compounds of Formula (I) wherein R² is aryl or heteroaryl and R⁶ is alkoxy may be prepared according to General Reaction Scheme XIV. Compound 14-3 is an example of Formula (I) wherein R² is aryl or heteroaryl, and R⁶ is alkoxy. Fluoro compound 14-1 with the amine suitably protected with for example, a BOC group or phthalimide group is subjected to aromatic S_{N}² conditions with —F as the leaving group and the corresponding oxy anion as the nucleophile. For example, with a sodium alkyl oxide in a polar solvent with heat to give substituent substituted product 14-2. The protecting group is then removed under appropriate conditions. For example, the BOC is removed under acidic conditions such as HCl or TFA in dioxane or the phthalimide group is removed under basic nucleophilic conditions such as hydrazine hydrate in ethanol with heat to afford R⁶-substituted amine 14-3 of Formula (I).

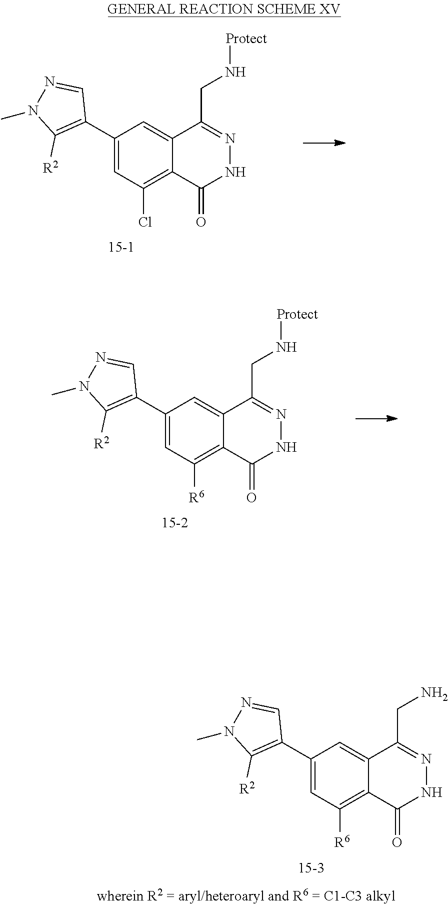

Compounds of Formula (I) wherein R² is aryl or heteroaryl and R⁶ is C1-C3 alkyl may be prepared according to General Reaction Scheme XV. Compound 15-3 is an example of Formula (I) wherein R² is aryl or heteroaryl, and R⁶ is C1-C3 alkyl. Chloro compound 15-1 with the amine suitably protected with for example, a BOC group or phthalimide group, is coupled to the appropriate C1-C3 trialkylborane under palladium catalyzed cross coupling conditions, for example Suzuki-Miyaura coupling conditions, to supply the corresponding R⁶-substituted coupling product 15-2. The protecting group is then removed under appropriate conditions. For example, the BOC is removed under acidic conditions such as HCl in dioxane or TFA in dioxane and the phthalimide group is removed under basic nucleophilic conditions such as hydrazine hydrate in ethanol with heat to afford R-substituted amine 15-3 of Formula (I).

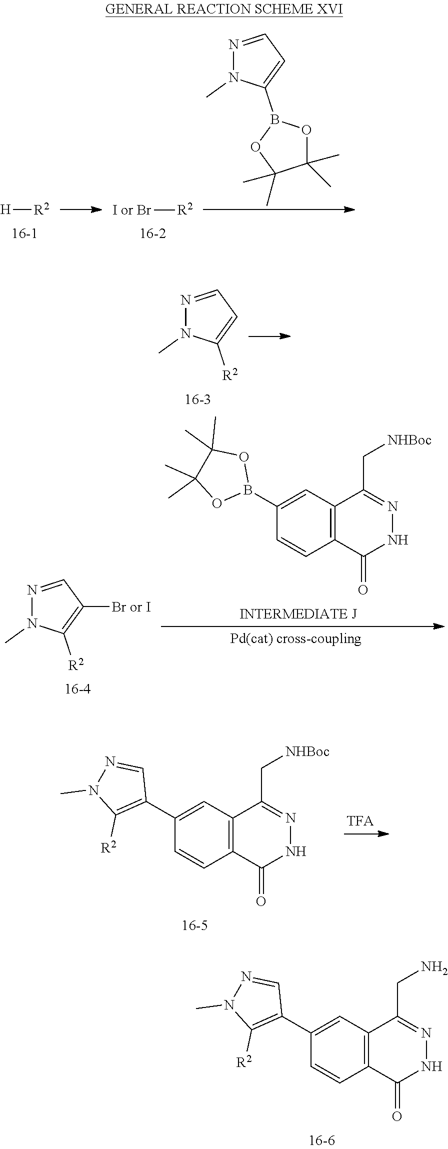

Compounds of Formula (I) wherein R¹ is 1-methyl-5-R²-1H-pyrazole-4-yl and R² is alkyl, aryl or heteroaryl, may be prepared according to General Reaction Scheme XVI. Compound 16-6 is an example of Formula (I) wherein R¹ is 1-methyl-5-R²-1H-pyrazole-4-yl, R² is alkyl, aryl or heteroaryl and R^{1a} and R^{3b} are H. H—R² 16-1 is halogenated, for example with a halogenating agent such N-bromosuccinimide or N-iodosuccinimide under palladium catalyzed conditions such as palladium acetate in the presence of an acid such as p-toluenesulfonic acid in a solvent such as dichloroethane under elevated temperature for example 70° C. to give halide 16-2. Bromo or iodo compound 16-2 is subjected to palladium-mediated cross coupling conditions, for example Suzuki conditions, with 1-methyl-5-(4,4,5,5-tetramethyl-1,3,2-dioxaborolan-2-yl)-1H-pyrazole to give coupled product 16-3. 1-methyl-5-R²-1H-pyrazole 16-3 is halogenated for example with a halogenating agent such N-bromosuccinimide or N-iodosuccinimide in a polar solvent such as acetonitrile to give 4-halo-1-methyl-5-R²-1H-pyrazole compound 16-4. 4-bromo-1-methyl-5-R²-1H-pyrazole 16-4 is coupled to Intermediate J under palladium-mediated cross coupling conditions, for example Suzuki conditions, to provide N-Boc-R²-substituted coupling product 16-5. The coupling product 16-5 is subjected to acidic conditions to remove the Boc group, for example TFA, to afford R²-substituted amine 16-6 of Formula (I).

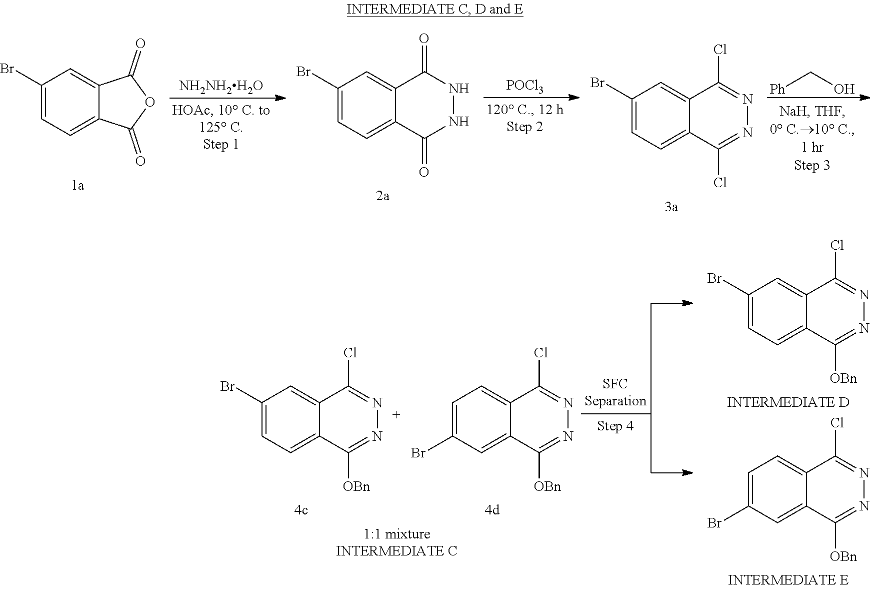

Step 1: A mixture of 5-bromoisobenzofuran-1,3-dione 1a (55.0 g, 242 mmol, 1.00 eq.) and acetic acid (165 mL) was stirred at 125° C. for 1 hour. After such time the mixture was cooled to 10° C. and hydrazine hydrate (12.7 g, 254 mmol, 12.4 mL, 1.05 eq.) was added dropwise, resulting in the formation of a thick white precipitate. Additional acetic acid (55 mL) was added and the mixture stirred at 125° C. for a further 30 mins. After such time time the mixture was cooled, diluted with acetic acid (150 mL) and filtered. The filter cake was washed with acetic acid (50 mL×3), dried and then dissolved in a 5% (w/w) sodium hydroxide solution (800 mL). The solution was acidified with acetic acid (200 mL) to give a thick white precipitate which was filtered. The filter cake was washed with water (50 mL×3) followed by methanol then dried in vacuo to give 6-bromo-2,3-dihydrophthalazine-1,4-dione 2a (45.6 g, crude) as a white solid. This solid was then used in the next step without further purification. ¹H NMR (400 MHz, DMSO-d₆) δ=8.17 (d, J=2.0 Hz, 1H), 8.01-7.97 (m, 1H), 7.95-7.89 (m, 1H).

Step 2: A solution of 6-bromo-2,3-dihydrophthalazine-1,4-dione 2a (20.0 g, crude) in phosphorus oxychloride (330 g, 2.15 mol, 200 mL) was stirred at 120° C. for 12 hours. After such time the reaction mixture was concentrated under reduced pressure and the residue dissolved in dichloromethane (150 mL) and added dropwise to ice-water. The mixture was then extracted with dichloromethane (300 mL×3) and the combined organic layers were washed with sodium bicarbonate aqueous solution (200 mL×5), brine (200 mL×2), dried over sodium sulfate, filtered and concentrated under reduced pressure to give 6-bromo-1,4-dichloro-phthalazine 3a (14.5 g, crude) as a yellow solid. This solid was then used in the next step without further purification. ¹H NMR (400 MHz, CDCl₃) δ=8.49 (d, J=1.2 Hz, 1H), 8.23-8.15 (m, 2H).

Step 3: A solution of benzyl alcohol (4.59 g, 42.4 mmol, 4.41 mL) and sodium hydride (3.77 g, 94.3 mmol, 60% dispersion in mineral oil) in THF (30 mL) was stirred at 0° C. for 0.5 hour. The mixture was then added dropwise to a solution of 6-bromo-1,4-dichloro-phthalazine 3a (13.1 g, crude) in THF (80 mL) at 0° C. The reaction mixture was warmed to 10° C. and stirred at 10° C. for 1 hour. After such time the reaction mixture was diluted with water (100 mL) and extracted with ethyl acetate (150 mL×3). The combined organic layers were washed with brine (100 mL), dried over anhydrous sodium sulfate, filtered and concentrated under reduced pressure. The residue was purified by column chromatography (SiO₂, petroleum ether:ethyl acetate 10-25%) to give Intermediate C, a 1:1 mixture of 4-benzyloxy-7-bromo-1-chloro-phthalazine 4c and 4-benzyloxy-6-bromo-1-chloro-phthalazine 4d (9.79 g, 28.0 mmol, 66% yield) as a yellow solid. ¹H NMR (400 MHz, CDCl₃) δ=8.39 (d, J=1.2 Hz, 1H), 8.35 (d, J=1.6 Hz, 1H), 8.17-8.10 (m, 1H), 8.08-8.05 (m, 1H), 8.04 (d, J=2.0 Hz, 1H), 8.00 (dt, J=1.6, 8.4 Hz, 1H), 7.59-7.53 (m, 4H), 7.47-7.36 (m, 6H), 5.70 (s, 4H).

Step 4: The regioisomers of Intermediate C, a 1:1 mixture of 4c and 4d (9.79 g, 28.0 mmol) were separated by SFC (column: DAICEL CHIRALPAK AD (250×30 mm, 10 μm); mobile phase: [0.1% NH₃H₂O MeOH]; B %: 0%-60%; 40 min) to give Intermediate D, 4-benzyloxy-7-bromo-1-chloro-phthalazine (2.40 g, 6.86 mmol) as a white solid and Intermediate E, 4-benzyloxy-6-bromo-1-chloro-phthalazine (2.54 g, 7.27) as a white solid. Intermediate D: 4-benzyloxy-7-bromo-1-chloro-phthalazine: ¹H NMR (400 MHz, CDCl₃) δ=8.36 (d, J=2.0 Hz, 1H), 8.13 (d, J=8.8 Hz, 1H), 7.99 (dd, J=2.0, 8.8 Hz, 1H), 7.58-7.54 (m, 2H), 7.46-7.36 (m, 3H), 5.70 (s, 2H). LCMS [M+1]⁺351.0. Intermediate E: 4-benzyloxy-6-bromo-1-chloro-phthalazine: ¹H NMR (400 MHz, CDCl₃) δ=8.39 (d, J=1.6 Hz, 1H), 8.10-8.01 (m, 2H), 7.61-7.54 (m, 2H), 7.48-7.35 (m, 3H), 5.70 (s, 2H). LCMS [M+1]⁺351.0.

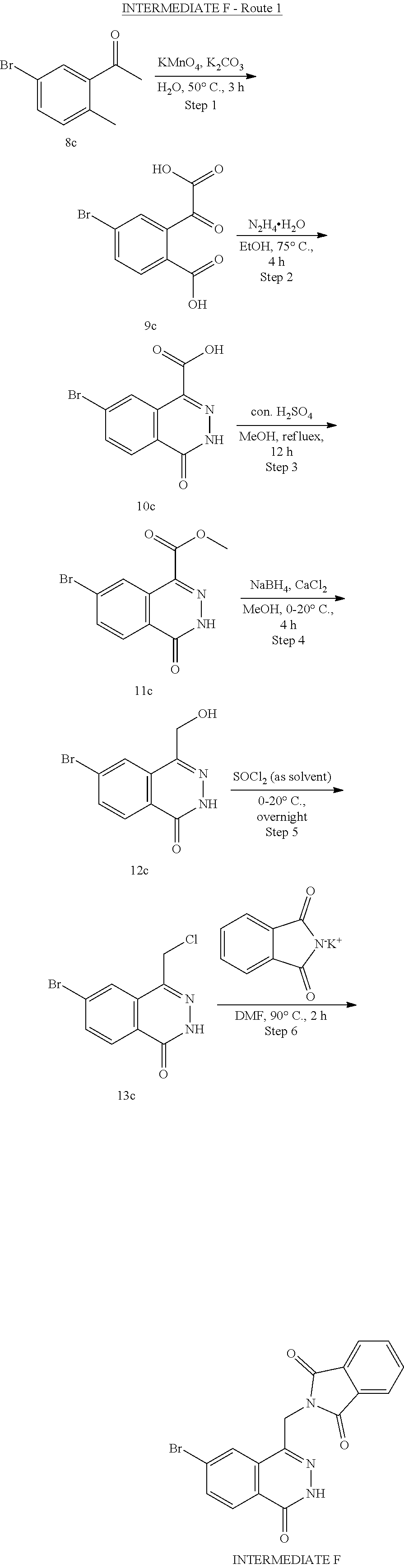

Step 1: To a solution of 1-(5-bromo-2-methyl-phenyl)ethenone 8c (100 g, 445 mmol, 1.00 eq.) in water (1.00 L) was added potassium carbonate (92.4 g, 668 mmol, 1.50 eq.) and potassium permanganate (493 g, 3.12 mol, 7.00 eq.). The mixture was stirred at 50° C. for 3 hours before ethanol (1.00 L) was added and the resulting mixture stirred at 50° C. for a further 30 minutes. After such time the solid was filtered and the filtrate pH adjusted to pH 2 with conc. hydrochloric acid (500 mL). The mixture was then extracted with ethyl acetate (1.00 L), the organic layer separated then concentrated in vacuo to give 4-bromo-2-oxalo-benzoic acid 9c (278 g, 997 mmol, 75% yield) as a white solid which was used in the next step without further purification. LCMS [M+1]⁺=271.1.

Step 2: To a solution of 4-bromo-2-oxalo-benzoic acid 9c (382 g, 1.27 mol) in ethyl alcohol (3.00 L) was added hydrazine hydrate (71.2 g, 1.39 mol, 69.1 mL). The mixture was stirred at 75° C. for 4 hours and the formed precipitate was filtered, washed with ethyl alcohol (500 mL) and dried to give 7-bromo-4-oxo-3H-phthalazine-1-carboxylic acid 10c (280 g, 1.03 mol, 81% yield) as a white solid which was used in the next step without further purification. ¹H NMR (400 MHz, DMSO-d₆) δ=8.72-8.81 (m, 1H), 8.11-8.21 (m, 1H), 7.95-8.09 (m, 1H).

Step 3: To a solution of 7-bromo-4-oxo-3H-phthalazine-1-carboxylic acid 10c (200 g, 675 mmol) in methyl alcohol (2.00 L) was added sulfuric acid (131 g, 1.31 mol, 71.0 mL) and the reaction mixture stirred at 65° C. for 24 hours. After such time the cooled reaction mixture was filtered and the filter cake dried under reduced pressure to give methyl 7-bromo-4-oxo-3H-phthalazine-1-carboxylate 11b (216 g, crude) as a white solid which was used in the next step without further purification. LCMS [M+1]⁺=283.0; ¹H NMR (400 MHz, DMSO-d₆) δ=13.31 (s, 1H), 8.72 (s, 1H), 8.16-8.18 (d, J=8.4 Hz, 1H), 8.03-8.05 (d, J=8.4 Hz, 1H), 3.91 (s, 3H).

Step 4: A stirred solution of methyl 7-bromo-4-oxo-3H-phthalazine-1-carboxylate 11c (159 g, 494 mmol) in ethyl alcohol (1.50 L) was treated portion wise with sodium borohydride (48.6 g, 1.29 mol, 2.60 eq) at 0° C. To this mixture was added a solution of calcium chloride (65.8 g, 593 mmol, 1.20 eq). The mixture was then stirred for 2 hours at 0° C. and a further 1 hour at 20° C. After such time the reaction mixture was concentrated under reduced pressure, and the residue was suspended in water (800 mL), the pH was adjusted to pH 5 with 1N hydrochloric acid (300 mL) and the precipitate was filtered, washed with water (300 mL×3) and dried to give 6-bromo-4-(hydroxymethyl)-2H-phthalazin-1-one 12c (162 g, crude) as a yellow solid. LCMS [M+1]⁺=255.0; ¹H NMR (400 MHz, DMSO-d₆) δ=12.66 (s, 1H), 8.30 (d, J=1.6 Hz, 1H), 8.16 (d, J=8.8 Hz, 1H), 8.01 (dd, J=8.4, 2.0 Hz, 1H), 5.58 (t, J=5.6 Hz, 1H), 4.67 (d, J=6.0 Hz, 2H).

Step 5: 6-bromo-4-(hydroxymethyl)-2H-phthalazin-1-one 12c (162 g, crude) was dissolved in thionyl chloride (1.00 L) and the mixture was stirred at 70° C. for 2 hours then concentrated under reduced pressure (35° C.). The concentrated residue was dissolved in dichloromethane (1.00 L) and concentrated to dryness to give 6-bromo-4-(chloromethyl)-2H-phthalazin-1-one 13c (154 g, crude) as a white solid which was used in the next step without further purification. LCMS [M+1]⁺=274.8; ¹H NMR (400 MHz, DMSO-d₆) δ=12.92 (s, 1H), 8.30 (s, 1H), 8.18-8.20 (d, J=7.6 Hz, 1H), 8.06-8.08 (t, J=8.8 Hz, 1H), 5.07 (s, 2H).

Step 6: To a solution of 6-bromo-4-(chloromethyl)-2H-phthalazin-1-one 13c (148 g, crude) in DMF (1.5 L) was added (1,3-dioxoisoindolin-2-yl)potassium (121 g, 653 mmol). The reaction mixture was stirred at 90° C. for 2 hours and then cooled to 25° C. The formed precipitate was filtered and washed with DMF (200 mL×2) and the filter cake triturated with water (1.00 L), filtered and dried to give Intermediate F, 2-[(7-bromo-4-oxo-3H-phthalazin-1-yl)methyl]isoindoline-1,3-dione (162 g, 413 mmol, 76% yield) as a white solid. ¹H NMR (400 MHz, DMSO-d₆) δ=12.59 (s, 1H), 8.43 (d, J=1.2 Hz, 1H), 8.18 (d, J=8.4 Hz, 1H), 8.07 (dd, J=1.6, 8.4 Hz, 1H), 7.97-7.93 (m, 2H), 7.92-7.86 (m, 2H), 5.19 (s, 2H). LCMS [M+1]: 383.9.

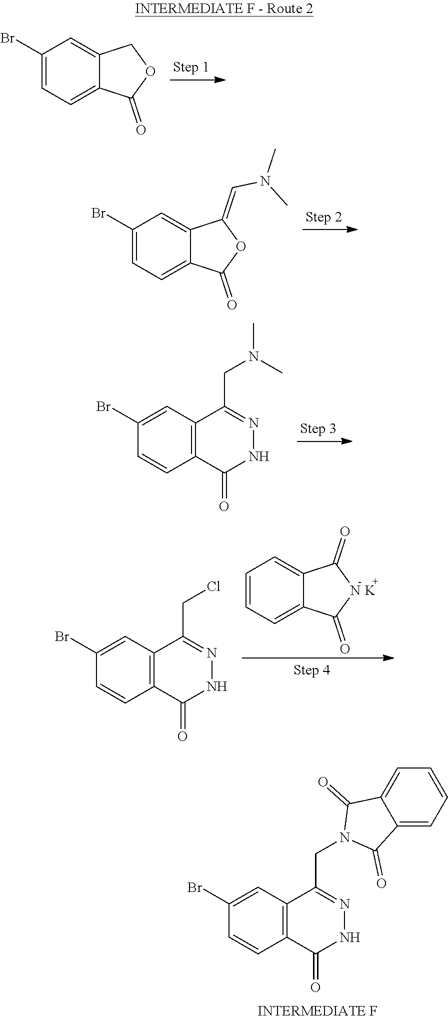

Step 1: A mixture of 5-bromoisobenzofuran-1 (3H)-one (50.0 g, 235 mmol, 1.00 eq), DMF-DMA (180 g, 1.51 mol, 201 mL, 6.44 eq) and t-BuOK (2.63 g, 23.5 mmol, 0.10 eq) was degassed and purged with N₂ 3 times and then stirred at 110° C. for 20 h under a N₂ atmosphere. After such time the reaction mixture concentrated under reduced pressure to remove the DMF-DMA and the formed residue was stirred in petroleum ether (100 mL) at 25° C. for 30 mins. The formed solid was filtered and the filter cake stirred in ethyl acetate (200 mL) at 80° C. for 12 h, filtered and the filter cake was dried under reduced pressure to give (Z)-5-bromo-3-((dimethylamino)methylene)isobenzofuiran-1 (3H)-one (39.08 g, 120 mmol, 51% yield, 82% purity) as a red solid. LCMS [M+1]⁺=270.1; ¹H NMR (400 MHz, DMSO-d₆) δ=797 (d, J=1.2 Hz, 1H), 7.61-7.59 (d, J=8.0, 1H), 7.30-7.27 (dd, J=8.0 & 1.2 Hz, 1H), 3.10 (s, 6H).

Step 2: To a mixture of (Z)-5-bromo-3-((dimethylamino)methylene)isobenzofuran-1 (3H)-one (39.0 g, 119 mmol, 82.0% purity, 1.00 eg) in EtOH (650 mL) was added NH₂NH₂·H₂O (12.5 g, 245 mmol, 12.1 mL, 2.05 eq) at 25° C. The mixture was degassed with N₂ then stirred at 25° C. for 0.5 h and then at 70° C. for 12 h. After such time the reaction mixture was filtered and the solid was dried to give 6-bromo-4-((dimethylamino)methyl)phthalazin-1 (2H)-one (30.0 g, 105 mmol, 88% yield, 99% purity) as a yellow solid. LCMS [M+1]⁺282.1; ¹H NMR (400 MHz, DMSO-d₆) δ 12.6 (s, 1H), 8.33 (s, 7H), 8.14-8.12 (d, J=8.4 Hz, 1H), 8.00-7.98 (i, 1H), 3.61 (s, 1H), 2.18 (s, 1H).

Step 3: A mixture of 6-bromo-4-((dimethylamino)methyl)phthalazin-1 (2H)-one (15.0 g, 53.2 mmol, 1.00 eq) in THF (187 mL) and degassed with N₂ 3 times before being cooled to ° C. Isobutyl cadonochloridate (8.71 g, 63.80 mmol, 8.38 mL, 1.20 eq) was then added dropwise and then the mixture stirred at 25° C. for 6 h under N₂. After such time the mixture was cooled to 0° C. before HCl (0.5 M, 250 mL) was added maintaining a temperature between 0° C. and 10° C. After the addition was complete the solid was filtered, washed with THF (30 mL×3) and dried to afford a 6-bromo-4-(chloromethyl)phthalazin-1 (2H)-one (11.0 g, 37.56 mmol, 71% yield, 93% purity) as a yellow solid. LCMS [M+1]+=256.1; ¹H NMR (400 MHz, DMSO-d₆) δ 12.9 (s, 1H), 8.29 (d, J=1.6 Hz, 1H), 8.19-8.17 (d, J=8.0 Hz, 1H), 8.06-80.4 (dd, J=8.0 Hz & 1.6 Hz, 1H), 5.06 (s, 2H).

Step 4: To a mixture of 6-bromo-4-(chloromethyl)phthalazin-1 (2H)-one (8.06 g, 27.5 mmol, 93% purity, 1.00 eq) in DMF (160 mL) was added (1,3-dioxoisoindolin-2-yl)potassium (5.61 g, 30.3 mmol, 1.10 eq) and stirred at 25° C. for 1 hr. After such time the mixture was washed with HCl (0.5 M, 100 mL), filtered and the solid washed with sat. NaHCO₃. (30 mL×2), pure water (30 mL×2) and then triturated with EtOH (15 mL) at 70° C. for 1 hr. The solid was then filtered and dried to give Intermediate F (8.30 g, 17.9 mmol, 65.0% yield, 83% purity) as a yellow solid. LCMS [M+1]⁺=384.1/386.1; ¹H NMR (400 MHz, DMSO-d₆) δ 12.6 (s, 1H), 8.43 (s, 1H), 8.18-8.16 (d, J=8.0 Hz, 1H), 8.08 (d, J=8.0 Hz, 1H), 7.95-7.89 (m, 4H), 5.18 (s, 2H).

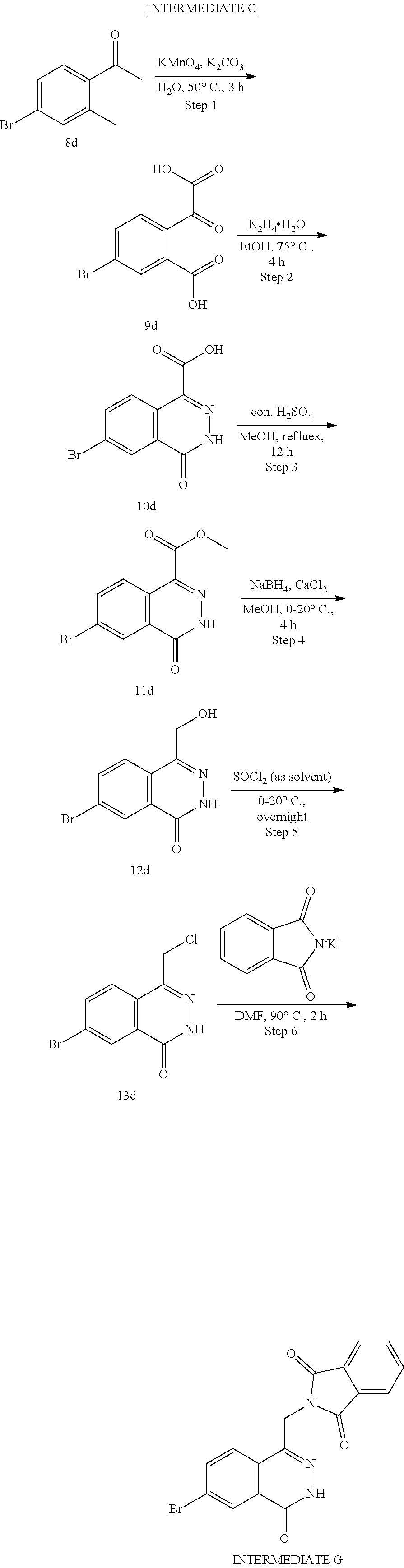

Step 1: To a solution of 1-(4-bromo-2-methyl-phenyl)ethenone 8d (10.0 g, 46.9 mmol, 1.00 eq.) in water (50 mL) was added potassium carbonate (9.73 g, 70.40 mmol, 1.50 eq.) and potassium permanganate (51.9 g, 329 mmol, 7.00 eq.). The mixture was stirred at 50° C. for 3 hours before ethanol (50 mL) was added and the resulting mixture stirred at 50° C. for a further 30 minutes. After such time the solid was filtered and the filtrate pH adjusted to pH 2 with conc. hydrochloric acid (5 mL). The mixture was then extracted with ethyl acetate (50 mL), the organic layer separated and concentrated in vacuo to give 5-bromo-2-oxalo-benzoic acid 9d (10.0 g, crude) as a white solid which was used in the next step without further purification. LCMS [M+1]+=273.0.

Step 2: To a solution of 5-bromo-2-oxalo-benzoic acid 9d (10.0 g, crude) in ethyl alcohol (120 mL) was added hydrazine hydrate (1.87 g, 36.6 mmol, 1.82 mL) and the mixture was stirred at 75° C. for 4 hours. After such time the formed precipitate was filtered and washed with ethyl alcohol (5 mL) and dried to give 6-bromo-4-oxo-3H-phthalazine-1-carboxylic acid 10d (7.50 g, 27.9 mmol, 59% yield) as a white solid. LCMS [M+1]⁺=269.0.

Step 3: To a solution of 6-bromo-4-oxo-3H-phthalazine-1-carboxylic acid 10d (7.50 g, 27.9 mmol, 1.00 eq.) in methyl alcohol (40 mL) was added sulfuric acid (16.7 g, 167 mmol, 9.10 mL, 6.00 eq.) and the reaction mixture stirred at 65° C. for 12 hours. After such time the reaction mixture was allowed to cool and the formed precipitate was filtered and dried to give methyl 6-bromo-4-oxo-3H-phthalazine-1-carboxylate 11d (7.00 g, 24.7 mmol, 89% yield) as a white solid. LCMS [M+1]⁺=282.9.

Step 4: A stirred solution of sodium borohydride (2.43 g, 64.29 mmol, 2.60 eq.) in ethyl alcohol (250 mL) was treated portion wise with methyl 6-bromo-4-oxo-3H-phthalazine-1-carboxylate 11d (7.00 g, 24.7 mmol, 1.00 eq.) at 0° C. To this mixture was added a solution of calcium chloride (3.29 g, 29.7 mmol, 1.20 eq.) in ethyl alcohol (250 mL) in a dropwise fashion. The mixture was then stirred for 3 hours at 0° C. and an additional hour at 20° C. After such time the mixture was concentrated under reduced pressure and the concentrated residue was suspended in water (30 mL) and the pH adjusted to pH 5 with 1N hydrochloric acid (5 mL). The formed precipitate was filtered, washed with water (5 mL×3) and triturated with ethyl alcohol (50 mL), filtered and dried to give 7-bromo-4-(hydroxymethyl)-2H-phthalazin-1-one 12d (6.00 g, 23.5 mmol, 95% yield) as a white solid. LCMS [M+1]+=255.0.

Step 5: 7-bromo-4-(hydroxymethyl)-2H-phthalazin-1-one 12d (6.00 g, 23.5 mmol) was dissolved in thionyl chloride (50 mL) at 0° C. The reaction mixture was stirred at 20° C. for 12 hours and then concentrated under reduced pressure (35° C.). The concentrated residue was dissolved in dichloromethane (20 mL) and concentrated to give 7-bromo-4-(chloromethyl)-2H-phthalazin-1-one 13d (5.50 g, crude) as a white solid which was used in the next step without further purification. LCMS [M+1]⁺=275.0.

Step 6: To a solution of 7-bromo-4-(chloromethyl)-2H-phthalazin-1-one 13d (5.50 g, crude) in DMF (60 mL) was added (1,3-dioxoisoindolin-2-yl)potassium (5.59 g, 30.2 mmol). The reaction mixture was stirred at 90° C. for 2 hours, then cooled to 25° C. and the formed precipitate was filtered and triturated with ethyl alcohol (150 mL) to give Intermediate G, 2-[(6-bromo-4-oxo-3H-phthalazin-1-yl)methyl]isoindoline-1,3-dione (5.00 g, 13.0 mmol, 65% yield) as a white solid. ¹H NMR (400 MHz, DMSO-d₆) δ=12.66 (s, 1H), 8.36 (d, J=2.0 Hz, 1H), 8.22-8.18 (m, 1H), 8.14-8.10 (m, 1H), 7.97-7.93 (m, 2H), 7.91-7.87 (m, 2H), 5.18 (s, 2H). LCMS [M+1]: 386.1.

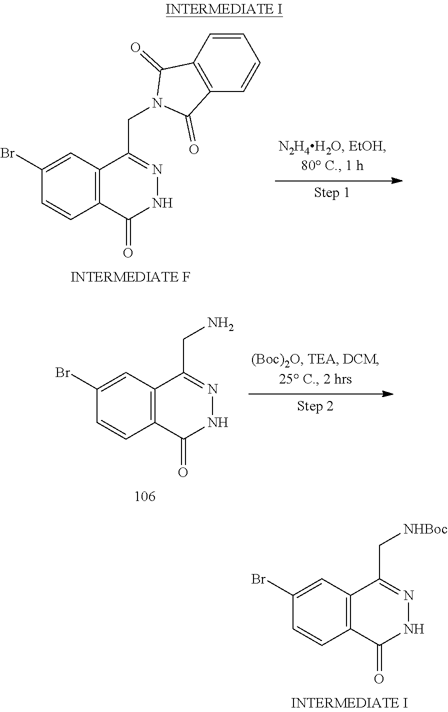 [SMILES: O=C1c2ccccc2C(=O)N1Cc1n[nH]c(=O)c2ccc(Br)cc12 ; NCc1n[nH]c(=O)c2ccc(Br)cc12 ; CC(C)(C)OC(=O)NCc1n[nH]c(=O)c2ccc(Br)cc12]

Step 1: A solution of intermediate F (3.00 g, 7.81 mmol, 1.00 eq.) and hydrazine hydrate (1.60 g, 31.2 mmol, 1.55 mL, 4.00 eq.) was stirred at 80° C. for 2 hours, cooled and concentrated under reduced pressure. The concentrated residue was then washed with water and triturated with ethyl alcohol at 25° C. to give 4-(aminomethyl)-6-bromo-2H-phthalazin-1-one 106 (1.95 g, 7.67 mmol, 98% yield) as a white solid. LCMS [M+1]⁺=256.1.

Step 2 To a solution of 4-(aminomethyl)-6-bromo-2H-phthalazin-1-one 106 (1.90 g, 7.48 mmol, 1.00 eq.) and triethylamine (2.27 g, 22.4 mmol, 3.12 mL, 3.00 eq) in dichloromethane (40.0 mL) was added di-tert-butyl dicarbonate (3.26 g, 15.0 mmol, 3.44 mL, 2.00 eq.). The mixture was stirred at 25° C. for 2 hours, filtered and concentrated under reduced pressure and theand the concentrated residue was triturated with dichloromethane (40 mL) then filtered and dried to give t-butyl-N-[(7-bromo-4-oxo-3H-phthalazin-1-yl)methyl]carbamate, Intermediate I (1.97 g, 5.56 mmol, 74% yield) as a white solid. LCMS [M+1]⁺=356.1. H NMR (400 MHz, DMSO-d₆) δ=12.71 (s, 1H), 8.26 (br s, 1H), 8.16 (br d, J=8.0 Hz, 1H), 8.02 (br d, J=8.0 Hz, 1H), 7.46 (br s, 1H), 4.41 (br d, J=4.4 Hz, 2H), 1.40 (br s, 9H).

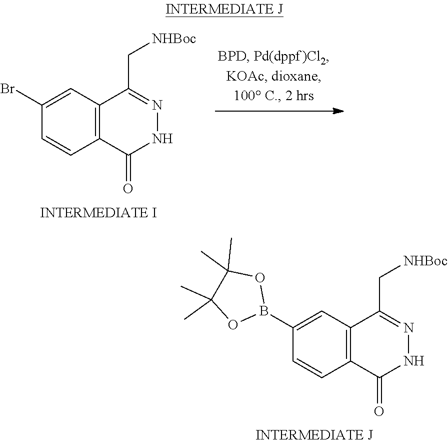 [SMILES: CC(C)(C)OC(=O)NCc1n[nH]c(=O)c2ccc(Br)cc12 ; CC(C)(C)OC(=O)NCc1n[nH]c(=O)c2ccc(B3OC(C)(C)C(C)(C)O3)cc12]

A mixture of Intermediate I (130.0, 275 mmol, 1.00 eq.), bis(pinacolato)diboron (BPD) (104.9 g, 412.9 mmol, 1.50 eq.), Pd(dppf)Cl₂ (20.1 g, 27.5 mmol, 0.10 eq.), KOAc (81.0 g, 825 mmol, 3.00 eq.) in dioxane (2.60 L) was degassed and purged with N₂. The mixture was then stirred at 100° C. for 2 hours. After such time the mixture was filtered, concentrated and the residue triturated with petroleum ether/ethyl acetate 10/1 (400 mL) at 25° C. for 1 hr. The solid was then filtered and dried to give Intermediate J as a brown solid (68.0 g, 162 mmol, 59% yield). LCMS [M+1]⁺=402.3; ¹H NMR (400 MHz, CDCl₃) δ=12.62 (s, 1H), 8.25 (s, 2H), 8.01-8.13 (m, 1H), 7.21-7.45 (m, 1H), 4.34-4.63 (m, 2H), 1.42 (s, 9H), 1.32 (s, 12H).

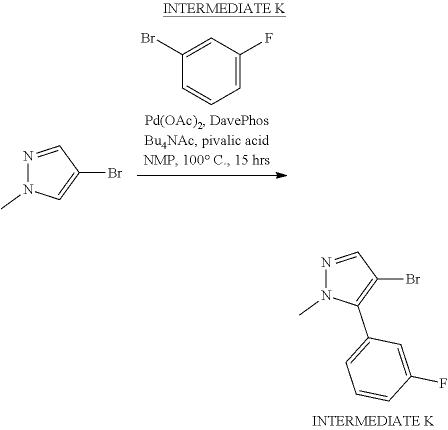

A solution of 4-bromo-1-methyl-pyrazole (500 mg, 3.11 mmol, 1.00 eq.) and the 1-bromo-3-fluoro-benzene (543 mg, 3.11 mmol, 346 μL, 1.00 eq.) in N-methylpyrrolidone (10 mL) was degassed with nitrogen. Then, palladium acetate (7.0 mg, 31.1 μmol, 0.10 eq.) and 2-(2-dicyclohexylphosphanylphenyl)-N,N-dimethyl-aniline (DavePhos) (24.0 mg, 62.1 μmol, 0.02 eq.) was added. To the resulting dark brown solution, tetrabutylammonium acetate (1.87 g, 6.21 mmol, 2 mL, 2.00 eq.) and pivalic acid (317 mg, 3.11 mmol, 357 μL, 1.00 eq.) were added and the resulting solution stirred at 100° C. for 15 hours. After the reaction was completed, the mixture was cooled. Ethyl acetate (100 mL) was added and the resulting mixture was washed with brine (3×100 mL). The organic phase was dried over anhydrous sodium sulfate and concentrated in vacuo to give a crude oil. The crude oil was purified by silica gel chromatography (petroleum ether/ethyl acetate 0-10%) to give 4-bromo-5-(3-fluorophenyl)-1-methyl-1H-pyrazole, Intermediate K (600 mg, 2.35 mmol, 76% yield) as a colorless oil. LCMS [M+1]⁺=255.0. ¹H NMR (400 MHz, CDCl₃) δ=7.56 (s, 1H), 7.50 (dt, J=6.0, 8.0 Hz, 1H), 7.24-7.18 (m, 2H), 7.18-7.13 (m, 1H), 3.85 (s, 3H).

The Intermediates A-1 to A-32 shown in Table I-I were prepared following the teachings of the General Reaction Schemes and the method to prepare INTERMEDIATE K.

TABLE I-I

| Intermediate | Structure | Spectral Data |
|---|---|---|
| A-1 | 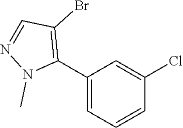 | 4-bromo-5-(3-chlorophenyl)-1-methyl-1H-pyrazole LCMS [M + 1]⁺ = 353.0; ¹H NMR (400 MHz, CDCl₃) δ = 7.54 (s, 1H), 7.47-7.43 (m, 2H), 7.42-7.40 (m, 1H), 7.30 (dt, J = 1.6, 4.4 Hz, 1H), 3.83 (s, 3H) |
| A-2 | 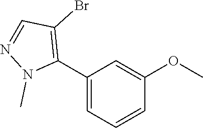 | 4-bromo-5-(3-methoxyphenyl)-1-methyl-1H-pyrazole LCMS [M + 1]⁺ = 267.0; ¹H NMR (400 MHz, DMSO-d₆) δ = 7.93 (s, 1H), 7.51 (s, 1H), 7.50-7.44 (t, J = 8.4, 1H), 7.11-7.07 (m, 1H), 7.05-7.03 (m, 1H), 3.83 (s, 3H), 3.82 (s, 3H) |
| A-3 | 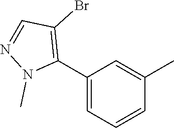 | 4-bromo-5-(3-methylphenyl)-1-methyl-1H-pyrazole LCMS [M + 1]⁺ = 251.0; ¹H NMR (400 MHz, CDCl₃) δ = 7.50 (S, 1H), 7.39-7.33 (m, 1H), 7.25 (br d, J = 7.6 Hz, 1H), 7.21-7.14 (m, 2H), 3.79 (s, 3H), 2.41 (s, 3H) |
| A-4 | 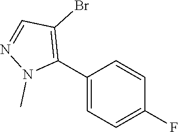 | 4-bromo-5-(4-fluorophenyl)-1-methyl-1H-pyrazole ¹H NMR (400 MHz, DMSO-d₆) δ = 7.65 (s, 1H), 7.58-7.54 (m, 2H), 7.42-7.38 (m, 2H), 3.35 (s, 3H) |
| A-5 | 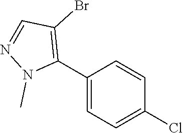 | 4-bromo-5-(4-chloropheny1)-1-methyl-1H-pyrazole LCMS [M + 1]⁺ = 272.9; ¹H NMR (400 MHz, CDCl₃) δ = 3.82 (s, 3 H) 7.34-7.38 (d, J = 8.8 Hz, 2 H) 7.48-7.52 (d, J = 8.8 Hz, 2 H) 7.55 (s, 1 H) |
| A-6 | 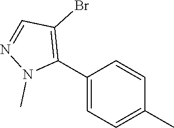 | 4-bromo-5-(4-methylpheny1)-1-methyl-1H-pyrazole LCMS [M + 1]⁺ = 251.0 |
| A-7 | 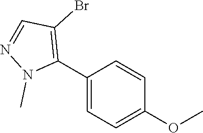 | 4-bromo-5-(4-methoxyphenyl)-1-methyl-1H-pyrazole LCMS [M + 1]⁺ = 269.0; ¹H NMR (400 MHz, CDCl₃ - d) δ = 7.53 (s, 1H), 7.36-7.32 (m, 2H), 7.05-7.01 (m, 2H), 3.88 (s, 3H), 3.81 (s, 3H) |
| A-8 | 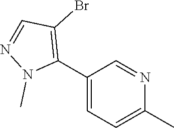 | 5-(4-bromo-1-methyl-1H-pyrazol-5-y1)-2-methylpyridine LCMS [M + 1]⁺ = 252.0; ¹H NMR (400 MHz, CDCl₃) δ = 8.56 (d, J = 2.4 Hz, 1H), 7.65 (dd, J = 2.4, 8.0 Hz, 1H), 7.56 (s, 1H), 7.32 (d, J = 8.0 Hz, 1H), 3.84 (s, 3H), 2.65 (s, 3H) |
| A-9 | 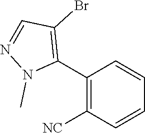 | 2-(4-bromo-1-methyl-1H-pyrazol-5-y1)-benzonitrile ¹H NMR (400 MHz, CDCl₃) δ = 7.86 (dd, J = 0.8, 7.6 Hz, 1H), 7.76 (dt, J = 1.2, 7.6 Hz, 1H), 7.63 (dt, J = 1.2, 7.6 Hz, 1H), 7.60 (s, 1H), 7.49 (dd, J = 0.8, 7.6 Hz, 1H), 3.81 (s, 3H) |
| A-10 | 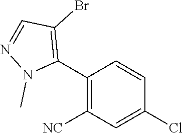 [SMILES: Cn1ncc(Br)c1-c1ccc(Cl)cc1C#N] | 2-(4-bromo-1-methyl-1H-pyrazol-5-yl)-5-chlorobenzonitrile LCMS [M + 1]⁺ = 297.9; ¹H NMR (400 MHz, CDCl₃) δ = 7.84 (d, J = 2.0 Hz, 1 H) 7.73 (dd, J = 8,4, 2.0 Hz, 1 H) 7.60 (s, 1 H) 7.43 (d, J = 8.4 Hz, 1 H) 3,81 (s, 3 H) |
| A-11 | 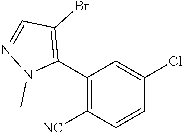 [SMILES: Cn1ncc(Br)c1-c1cc(Cl)ccc1C#N] | 2-(4-bromo-1-methyl-1/-pyrazol-5-y1)-4-chlorobenzonitrile ¹H NMR (400 MHz, DMSO-d₆) δ = 8.13 (d, J = 8.4 Hz, 1H), 7.93- 7.90 (d, J = 2.0 Hz, 1H), 7.87 (dd, J = 2.0, 8.4 Hz, 1H), 7.76 (s, 1H), 3.75 (s, 3H) |
| A-12 | 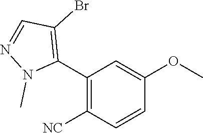 [SMILES: COc1ccc(C#N)c(-c2c(Br)cnn2C)c1] | 2-(4-bromo-1-methyl-1H-pyrazol-5-y1)-4-methoxybenzonitrile LCMS [M + 1]⁺ = 294.1; ¹H NMR (400 MHz, CDCl₃) δ = 7.77 (d, J = 8.8 Hz, 1H), 7.59 (s, 1H), 7.09 (dd, J = 2.8, 8.8 Hz, 1H), 6.95 (d, J = 2.8 Hz, 1H), 3.92 (s, 3H), 3.82 (s, 3H) |
| A-13 | 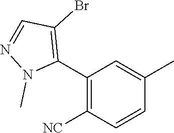 [SMILES: Cc1ccc(C#N)c(-c2c(Br)cnn2C)c1] | 2-(4-bromo-1-methyl-1H-pyrazol-5-y1)-4-methylbenzonitrile LCMS [M + 1]⁺ = 276.0; ¹H NMR (400 MHz, CDCl₃) δ = 7.73 (d, J = 8.0 Hz, 1H), 7.59 (s, 1H), 7.42 (dd, J = 0.8, 8.0 Hz, 1H), 7.28 (d, J = 2.8 Hz, 1H), 3.80 (s, 3H), 2.51 (s, 3H) |
| A-14 | 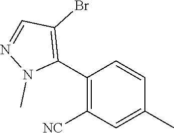 [SMILES: Cc1ccc(-c2c(Br)cnn2C)c(C#N)c1] | 2-(4-bromo-1-methyl-1H-pyrazol-5-y1)-5-methylbenzonitrile LCMS [M + 1]⁺ = 276.1; ¹H NMR (400 MHz, CDCl₃) δ = 7.58 (s, 1H), 7.50 (s, 1H), 7.48 (br d, J = 8.0 Hz, 1H), 7.28 (d, J = 8.0 Hz, 1H), 3.72 (s, 3H), 2.42 (s, 3H) |
| A-15 | 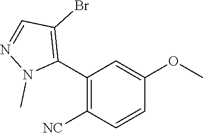 [SMILES: COc1ccc(C#N)c(-c2c(Br)cnn2C)c1] | 2-(4-bromo-1-methyl-1H-pyrazol-5-y1)-6-methoxybenzonitrile LCMS [M + 1]⁺ = 292.1; ¹H NMR (400 MHz, DMSO-d₆) δ = 7.87- 7.80 (m, 1H), 7.74 (s, 1H), 7.44 (d, J = 8.8 Hz, 1H), 7.19 (d, J = 7.6 Hz, 1H), 3.99 (s, 3H), 3.71 (s, 3H) |
| A-16 | 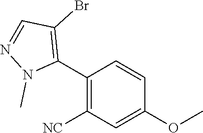 [SMILES: COc1ccc(-c2c(Br)cnn2C)c(C#N)c1] | 2-(4-bromo-1-methyl-1H-pyrazol-5-y1)-5-methoxybenzonitrile LCMS [M + 1]⁺ = 294.1; ¹H NMR (400 MHz, CDCl₃) δ = 7.50 (s, 1H), 7.30 (d, J = 8.8 Hz, 1H), 7.24 (d, J = 2.8 Hz, 1H), 7.21-7.17 (m, 1H), 3.84 (s, 3H), 3.71 (s, 3H). |
| A-17 | 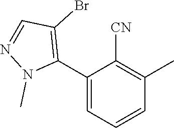 [SMILES: Cc1cccc(-c2c(Br)cnn2C)c1C#N] | 2-(4-bromo-1-methyl-1H-pyrazol-5-y1)-6-methylbenzonitrile LCMS [M + 1]⁺ = 278.0; ¹H NMR (400 MHz, DMSO-d₆) δ = 7.80 (t, J = 7.6 Hz, 1H), 7.74 (s, 1H), 7.66 (d, J = 7.6 Hz, 1H), 7.48 (d, J = 7.6 Hz, 1H), 3.70 (s, 3H), 2.58 (s, 3H) |
| A-18 | 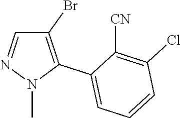 [SMILES: Cn1ncc(Br)c1-c1cccc(Cl)c1C#N] | 2-(4-bromo-1-methyl-1H-pyrazol-5-y1)-6-chlorobenzonitrile LCMS [M + 1]⁺ = 298.0; ¹H NMR (400 MHz, DMSO-d₆) δ = 7.98 (dd, J = 1.2, 6.8 Hz, 1H), 7.94 (d, J = 7.6 Hz, 1H), 7.77 (s, 1H), 7.69 (dd, J = 1.2 Hz, 1H), 3.75 (s, 3H) |
| A-19 | 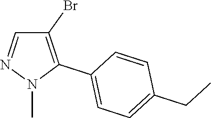 | 4-bromo-5-(4-ethylphenyl)-1-methyl-1H-pyrazole LCMS [M + 1]⁺ = 265.1; ¹H NMR (400 MHz, CDCl₃) δ = 7.54 (s, 1H), 7.33 (m, 4H), 3.82 (s, 3H), 2.81-2.71 (m, 2H), 1.31 (t, J = 7.6 Hz, 3H) |
| A-20 | 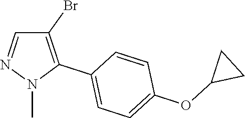 [SMILES: Cn1ncc(Br)c1-c1ccc(OC2CC2)cc1] | 4-bromo-5-(4-cyclopropoxyphenyl)-1-methyl-1H-pyrazole LCMS [M + 1]⁺ =295.0; ¹H NMR (400 MHz, CDCl₃) δ = 7.58 (s, 1H), 7.36-7.33 (m, 2H), 7.21-7.18 (m, 2H), 3.84 (s, 3H), 3.83- 3.78 (m, 1H), 0.87-0.84 (m, 2H), 0.83 (m, 2H) |
| A-21 | 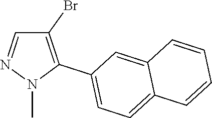 [SMILES: Cn1ncc(Br)c1-c1ccc2ccccc2c1] | 4-bromo-1-methyl-5-(naphthalen-2-y1)-1H-pyrazole LCMS [M + 1]⁺ = 289.1; ¹H NMR (400 MHz, CDCl₃) δ = 7.98 (d, J = 8.4 Hz, 1H), 7.95-7.87 (m, 3H), 7.63-7,55 (m, 3H), 7.50 (dd, J = 1,6, 8.4 Hz, 1H), 3.88 (s, 3H) |
| A-22 | 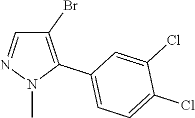 | 4-bromo-5-(3,4-dichlorophenyl)-1-methyl-1H-pyrazole LCMS [M + 1]⁺ = 306.8; ¹H NMR (400 MHz, CDCl₃) δ = 7.60 (d, J = 8.4 Hz, 1H), 7.55 (s, 1H), 7.52 (d, J = 2.0 Hz, 1H), 7.27 (dd, J = 2.0 Hz, J = 8.4 Hz, 1H), 3.83 (s, 3H) |
| A-23 | 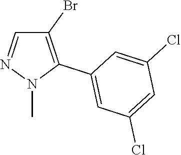 | 4-bromo-5-(3,5-dichlorophenyl)-1-methyl-1H-pyrazole LCMS [M + 1]⁺ =306.9; ¹H NMR (400 MHz, CDCl₃) δ = 7.55 (s, 1H), 7.48 (t, J = 1.8 Hz, 1H), 7.31 (d, J = 2.0 Hz, 2H), 3.84 (s, 3H) |
| A-24 | 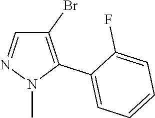 | 4-bromo-5-(2-fluorophenyl)-1-methyl-1H-pyrazole LCMS [M + 1]⁺ = 255.0; ¹H NMR (400 MHz, CDCl₃) δ = 7.57 (s, 1H), 7.54-7.47 (m, 1H), 7.41-7.37 (m, 1H), 7.30 (br t, J = 8.0 Hz, 1H), 7.26-7.20 (m, 1H), 3.79 (s, 3H) |
| A-25 | 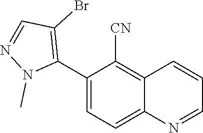 [SMILES: Cn1ncc(Br)c1-c1ccc2ncccc2c1C#N] | 6-(4-bromo-1-methyl-1H-pyrazol-5-yl)quinoline-5-carbonitrile LCMS [M + 1]⁺= 313.0; ¹H NMR (400 MHz, CDCl₃) δ = 9.15 (dd, J = 1.6, 4.0 Hz, 1H), 8.67 (dd, J = 0.8, 8.4 Hz, 1H), 8.49 (d, J = 8.8 Hz, 1H), 7.77 (d, J = 8.8 Hz, 1H), 7.73 (dd, J = 4.0, 8.4 Hz, 1H), 7.67 (s, 1H), 3.88 (s, 3H) |
| A-26 | 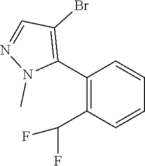 [SMILES: Cn1ncc(Br)c1-c1ccccc1C(F)F] | 4-bromo-5-(2-(difluoromethyl)phenyl)-1-methyl-1H-pyrazole LCMS [M + 1]⁺ = 287.0; ¹H NMR (400 MHz, CDCl₃) δ = 7.86- 7.80 (m, 1H), 7.69-7.60 (m, 2H), 7.58 (s, 1H), 7.32-7.28 (m, 1H), 6.59-6.27 (m, 1H), 3.65 (s, 3H) |
| A-27 | 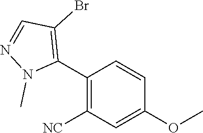 [SMILES: COc1ccc(-c2c(Br)cnn2C)c(C#N)c1] | 2-(4-bromo-1-methyl-1H-pyrazol-5-yl)-5-methoxybenzonitrile LCMS [M + 1]⁺ = 294.1; ¹H NMR (400 MHz, CDCl₃) δ = 7.50 (s, 1H), 7.30 (d, J = 8.8 Hz, 1H), 7.24 (d, J = 2.8 Hz, 1H), 7.21-7.17 (m, 1H), 3.84 (s, 3H), 3.71 (s, 3H). |
| A-28 | 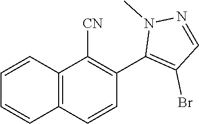 | 4-bromo-5-(1-chloronaphthalen-2-y1)-1-methyl-1H-pyrazole LCMS [M + 1]⁺ = 323.2; ¹H NMR (400 MHz, CDCl₃) δ = 8.41 (d, J = 8.4 Hz, 1H), 8.00-7.87 (m, 2H), 7.77-7.62 (m, 3H), 7.37 (d, J = 8.4 Hz, 1H), 3.77 (s, 3H) |
| A-29 | 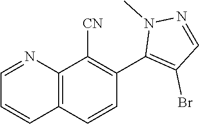 [SMILES: Cn1ncc(Br)c1-c1ccc2cccnc2c1C#N] | 7-(4-bromo-1-methyl-1H-pyrazol-5-yl)quinoline-8-carbonitrile LCMS [M + 1]⁺ = 313.2; ¹H NMR (400 MHz, CDCl₃) δ = 9.19 (dd, J = 1.6, 4.0 Hz, 1H), 8.34 (dd, J = 1.6, 8.4 Hz, 1H), 8.20 (d, J = 8.4 Hz, 1H), 7.72-7.60 (m, 3H), 3.89 (s, 3H) |
| A-30 | 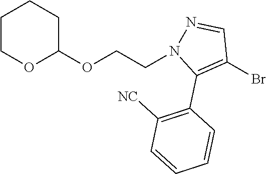 [SMILES: N#Cc1ccccc1-c1c(Br)cnn1CCOC1CCCCO1] | 2-(4-bromo-1-(2-((tetrahydro-2H-pyran-2-yl)oxy)ethyl)-1H- pyrazol-5-yl)benzonitrile LCMS [M − 83]⁺ = 292.1; ¹H NMR (400 MHz, CDCl₃) δ = = 7.84 (d, J = 7.9 Hz, 1H), 7.76-7.70 (m, 1H), 7.65 (s, 1H), 7.63-7.58 (m, 1H), 7.58-7.51 (m, 1H), 4.49-4.40 (m, 1H), 4.31-4.03 (m, 3H), 4.01-3.81 (m, 1H), 3.76-3.62 (m, 1H), 3.48-3.36 (m, 1H), 1.60-1.38 (m, 6H) |
| A-31 | 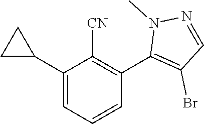 | 2-(4-bromo-1-methyl-1H-pyrazol-5-yl)-6-cyclopropylbenzonitrile LCMS [M + 1]⁺ =397.3; ¹H NMR (400 MHz, CD₃OD) δ = 8.23 (d, J = 8.4 Hz, 1H), 8.14 (s, 1H), 7.81-7.75 (m, 1H), 7.70 (d, J = 8.4 Hz, 1H), 7.57 (s, 1H), 7.50-7.45 (m, 1H), 7.30 (d, J = 8.0 Hz, 1H), 3.85 (s, 2H), 3.80 (s, 3H), 2.36-2.19 (m, 1H), 1.24-1.17 (m, 2H), 0.98-0.86 (m, 2H) |
| A-32 | 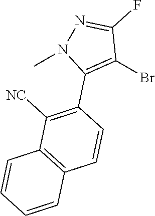 [SMILES: Cn1nc(F)c(Br)c1-c1ccc2ccccc2c1C#N] | 2-(4-bromo-3-fluoro-1-methyl-1H-pyrazol-5-y1)-1-naphthonitrile LCMS [M + 1]⁺ = 330.0; ¹H NMR (400 MHz, CDCl₃) δ = 8.33 (d, J = 8.4 Hz, 1H), 8.21 (d, J = 8.4 Hz, 1H), 8.01 (d, J = 7.6 Hz, 1H), 7.80 (dt, J = 1.2, 7.6 Hz, 1H), 7.76-7.70 (m, 1H), 7.50 (d, J = 8.4 Hz, 1H), 3.72 (s, 3H) |

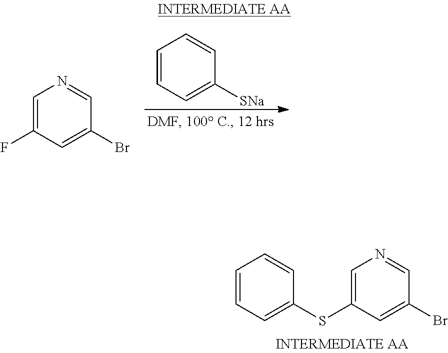

To a solution of 3-bromo-5-fluoro-pyridine (2.20 g, 12.5 mmol, 1.00 eq.) in dimethylformamide (50 mL) was added phenylsulfanylsodium (1.98 g, 15.0 mmol, 1.20 eq.) followed by stirring at 110° C. for 12 hours. After such time the reaction mixture was diluted with water (700 mL) and extracted with ethyl acetate (150 mL×3). The combined organic layers were washed with brine (300 mL), dried over anhydrous sodium sulfate, filtered and concentrated under reduced pressure. The residue was purified by column chromatography (SiO₂, petroleum ether:ethyl acetate 0-10%) to give 3-bromo-5-phenylsulfanyl-pyridine, Intermediate AA (1.31 g, 4.48 mmol, 35% yield) as a yellow oil. LCMS [M+1]⁺=268.0; ¹H NMR (400 MHz, MeOD) δ=8.44 (d, J=2.0 Hz, 1H), 8.31 (d, J=2.0 Hz, 1H), 7.73 (t, J=2.0 Hz, 1H), 7.50-7.46 (m, 2H), 7.45-7.41 (m, 3H).

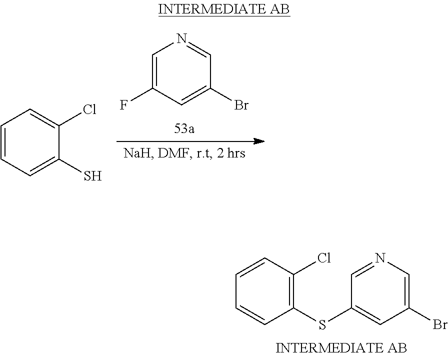

To a solution of 2-chlorobenzenethiol (296 mg, 2.05 mmol, 233 μL, 1.20 eq.) in DMF (2 mL) was added sodium hydride (82 mg, 2.05 mmol, 60% purity, 1.20 eq.) and 3-bromo-5-fluoro-pyridine (300 mg, 1.70 mmol, 1.00 eq.) and stirred at 25° C. for 2 hours. The reaction mixture was then quenched by addition water (10 mL) and then extracted with ethyl acetate (30 mL×3). The combined organic layers were washed with brine (50 mL×2), dried over anhydrous sodium sulfate, filtered and concentrated under reduced pressure. The residue was purified by prep-TLC (petroleum ether:ethyl acetate 20%) to give 3-bromo-5-(2-chlorophenyl)sulfanyl-pyridine, Intermediate AB (280 mg, 931 μmol, 54% yield) as a white solid. LCMS [M+1]⁺=302.0; ¹H NMR (400 MHz, CDCl₃-d) δ=8.56 (d, J=2.0 Hz, 1H), 8.45 (d, J=2.0 Hz, 1H), 7.73 (t, J=2.0 Hz, 1H), 7.51-7.47 (m, 1H), 7.33-7.28 (m, 2H), 7.26-7.22 (m, 1H).

The INTERMEDIATES AC to AG shown in Table I-IIa were prepared following the teachings of the General Reaction Schemes and the method to prepare INTERMEDIATE AB.

TABLE I-Ia

| Intermediate | Structure | Characterization |
|---|---|---|
| AC | 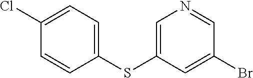 [SMILES: Clc1ccc(Sc2cncc(Br)c2)cc1] | 3-bromo-5-((4-chlorophenyl)thio)pyridine LCMS [M + 1]⁺ = 302.0 |
| AD | 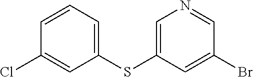 [SMILES: Clc1cccc(Sc2cncc(Br)c2)c1] | 3-bromo-5-((3-chlorophenyl)thio)pyridine LCMS [M + 1]⁺ = 302.0; ¹H NMR (400 MHz, CDCl₃) δ = 8.55 (d, J = 2.0 Hz, 1H), 8.46 (d, J = 1.6 Hz, 1H), 7.74 (t, J = 2.0 Hz, 1H), 7.40- 7.38 (m, 1H), 7.34-7.30 (m, 2H), 7.29-7.27 (m, 1H) |
| AE | 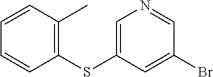 | 3-bromo-5-(o-tolylthio)pyridine LCMS [M + 1]⁺ = 280.0; ¹H NMR (400 MHz, CDCl₃) δ = 8.45 (br s, 1H), 8.29 (br s, 1H), 7.48 (s, 1H), 7.44 (d, J = 7.6 Hz, 1H), 7.37-7.31 (m, 2H), 7.26-7.21 (m, 1H), 2.40 (s, 3H) |
| AF | 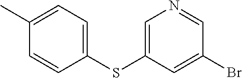 | 3-bromo-5-(p-tolylthio)pyridine LCMS [M + 1]⁺ = 281.0; ¹H NMR (400 MHz, DMSO-d₆) δ = 8.53 (d, J = 1.6 Hz, 1H), 8.41 (d, J = 2.4 Hz, 1H), 7.82 (q, J = 2.4 Hz, 1H), 7.49- 7.41 (m. 1H), 7.31-7.23 (m, 2H), 7.11-7.06 (m, 1H) |
| AG | 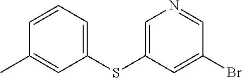 | 3-bromo-5-(m-tolylthio)pyridine LCMS [M + 1]⁺ = 279.9; ¹H NMR (400 MHz, CDCl₃) δ = 8.48 (d, J = 2.0 Hz, 1H), 8.40 (d, J = 1.6 Hz, 1H), 7.67-7.61 (m, 1H), 7.34-7.28 (m, 3H), 7.21-7.19 (m, 1H), 2.38 (s, 3H) |

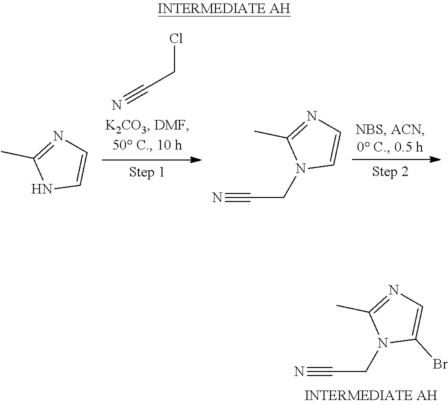

Step 1: To a solution of 2-methyl-1H-imidazole (1.00 g, 12.2 mmol, 1.00 eq.) in DMF (10 mL) was added potassium carbonate (1.68 g, 12.2 mmol, 1.00 eq.) and 2-chloroacetonitrile (920 mg, 12.2 mmol, 773 μL, 1.00 eq.) and the mixture stirred at 50° C. for 5 hours. After such time the reaction mixture was diluted with water (20 mL), extracted with ethyl acetate (20 mL×3) and the combined organic phases were washed with brine (20 mL), dried over anhydrous sodium sulfate, filtered and concentrated. The residue was purified by column chromatography (SiO₂, petroleum ether:ethyl acetate 10-100%) to give 2-(2-methylimidazol-1-yl)acetonitrile (460 mg, 3.80 mmol, 31% yield) as a yellow oil. ¹H NMR (400 MHz, CDCl₃) δ=6.98 (d, J=1.2 Hz, 1H), 6.94 (d, J=1.2 Hz, 1H), 4.79 (s, 2H), 2.47 (s, 3H).

Step 2: To a solution of 2-(2-methylimidazol-1-yl)acetonitrile (410 mg, 3.38 mmol, 1.00 eq.) in acetonitrile (10 mL) was added a solution of N-bromosuccinimide (542 mg, 3.05 mmol, 0.90 eq.) in acetonitrile (10 mL) dropwise at 0° C. The reaction mixture was stirred at 0° C. for 30 minutes and after such time the reaction mixture was quenched with water (2 mL) and extracted with ethyl acetate (2 mL×3). The combined organic phases were washed with brine (2 mL), dried over anhydrous sodium sulfate, filtered and concentrated to a residue. The residue was purified by column chromatography (SiO₂, petroleum ether:ethyl acetate 10-100%) followed by a second column (SiO₂, petroleum ether:ethyl acetate:methanol 1:1:0.4) to give 2-(5-bromo-2-methyl-imidazol-1-yl)acetonitrile, Intermediate AH (460 mg, 2.30 mmol, 67% yield) as a brown solid. ¹H NMR (400 MHz, CDCl₃) δ=6.97 (s, 1H), 4.82 (s, 2H), 2.53 (s, 3H).

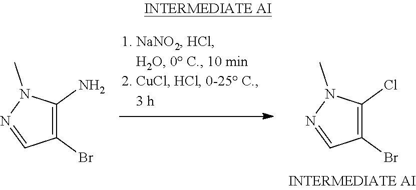

To a solution of 4-bromo-2-methyl-pyrazol-3-amine (0.20 g, 1.14 mmol, 1.00 eq.) in hydrochloric acid (12 M, 2 mL, 21.1 eq.) was slowly added a solution of sodium nitrite (86 mg, 1.25 mmol, 1.10 eq.) in water (1.8 mL) at 0° C. After stirring for 10 minutes, the mixture was added in portions to a solution of cuprous chloride (112 mg, 1.14 mmol, 27.2 μL, 1.00 eq.) in hydrochloric acid (12 M, 1.0 mL, 10.6 eq.). The reaction mixture was stirred at 25° C. for 3 hours. After such time the reaction mixture was diluted with water (5 mL) and extracted with ethyl acetate (5 mL×3). Combined organic phases were washed with aqueous sodium bicarbonate (5 mL), brine (5 mL), dried over anhydrous sodium sulfate, filtered and concentrated. The residue was purified by prep-TLC (petroleum ether:ethyl acetate 25%) to give 4-bromo-5-chloro-1-methyl-pyrazole, Intermediate AI (92 mg, 363 μmol, 31% yield) as a white solid. LCMS [M+1]⁺=197.0; ¹H NMR (400 MHz, CDCl₃-d) δ=7.48 (s, 1H), 3.88 (s, 3H).

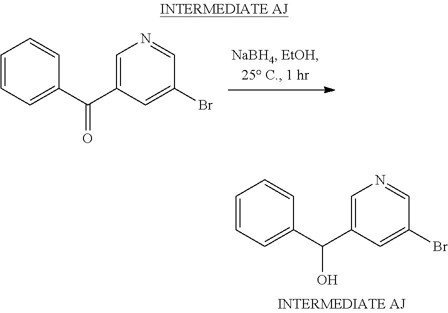 [SMILES: O=C(c1ccccc1)c1cncc(Br)c1 ; OC(c1ccccc1)c1cncc(Br)c1]

Sodium borohydride (22 mg, 572 μmol, 1.50 eq.) was added slowly to a solution of (5-bromo-3-pyridyl)-phenyl-methanone (100 mg, 381 μmol, 1.00 eq.) in ethyl alcohol (5 mL). After stirring at 25° C. for 2 hours the reaction was quenched with water (2 mL) and concentrated under vacuum. The residue was diluted with ethyl acetate (10 mL), washed with brine (10×3 mL), dried over anhydrous sodium sulfate, filtered and concentrated to give (5-bromopyridin-3-yl)(phenyl)methanol, Intermediate AJ (97 mg, 367 μmol, 96% yield) as a colorless oil. LCMS [M+1]⁺=263.9. ¹H NMR (400 MHz, CDCl₃) δ=8.53 (d, J=2.0 Hz, 1H), 8.48 (d, J=2.0 Hz, 1H), 7.89 (t, J=2.0 Hz, 1H), 7.43-7.38 (m, 1H), 7.38-7.31 (m, 4H), 5.85 (s, 1H), 2.85 (s, 1H).

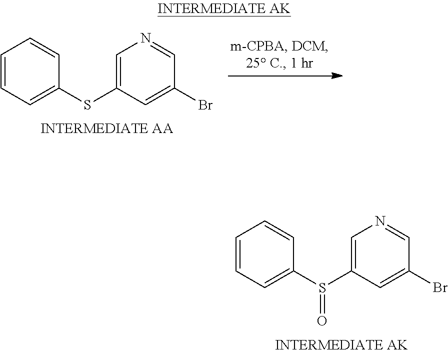 [SMILES: Brc1cncc(Sc2ccccc2)c1 ; O=S(c1ccccc1)c1cncc(Br)c1]

To a solution of 3-bromo-5-(phenylthio)pyridine, Intermediate AA (200 mg, 751 μmol, 1.00 eq.) in dichloromethane (4 mL) was added 3-chloroperoxybenzoic acid (153 mg, 751 μmol, 85.0% purity, 1.00 eq.). The resulting mixture was stirred at 25° C. under nitrogen for 1 hour. After such time sodium hydroxide aqueous solution (4 N, 40 mL) was added and the mixture extracted with dichloromethane (20 mL×2). The combined organic layers were washed with brine (5 mL×2), dried over sodium sulfate and concentrated. The residue was purified by column chromatography (SiO₂, petroleum ether:ethyl acetate 5-20%) to give 3-bromo-5-(phenylsulfinyl)pyridine, Intermediate AK (150 mg, 532 μmol, 70% yield) as a white solid. ¹H NMR (400 MHz, CDCl₃) δ=8.72 (d, J=2.0 Hz, 1H), 8.66 (d, J=2.0 Hz, 1H), 8.16 (t, J=2.0 Hz, 1H), 7.72-7.68 (m, 2H), 7.55-7.27 (m, 3H).

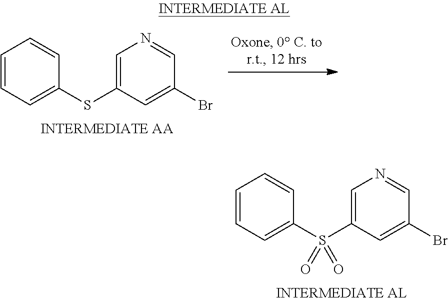 [SMILES: Brc1cncc(Sc2ccccc2)c1 ; O=S(=O)(c1ccccc1)c1cncc(Br)c1]

A solution of oxone (2.10 g, 3.42 mmol, 2.00 eq.) in water (10 mL) was added to a solution of 3-bromo-5-phenylsulfanyl-pyridine intermediate AA (500 mg, 1.71 mmol, 1.00 eq.) in THF (10 mL) and methyl alcohol (10 mL) at 0° C. The resulting mixture was stirred at 35° C. for 12 hours and after such time was filtered and the filtrate concentrated under reduced pressure. The formed residue was purified by reversed-phase HPLC (0.1% FA condition) to give 3-(benzenesulfonyl)-5-bromo-pyridine, Intermediate AL (300 mg, 1.01 mmol, 59% yield) as a white solid. ¹H NMR (400 MHz, DMSO-d₆) δ=9.13 (d, J=2.0 Hz, 1H), 9.01 (d, J=2.0 Hz, 1H), 8.66 (t, J=2.0 Hz, 1H), 8.12-8.08 (m, 2H), 7.77-7.72 (m, 1H), 7.69-7.64 (m, 2H).

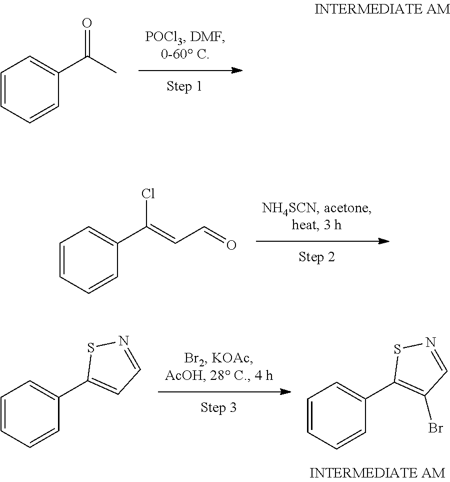

Step 1: Phosphorus oxychloride (4.71 g, 30.7 mmol, 2.9 mL, 1.23 eq.) was added dropwise to DMF (6 mL) at 0° C., and then the mixture was stirred at 0° C. for 10 minutes. After such time a solution of 1-phenylethanone (3.00 g, 25.0 mmol, 2.91 mL, 1.00 eq.) in DMF (25 mL) was added dropwise with stirring. The reaction mixture was then heated 3 hours at 60° C. After such time the solution was cooled to room temperature and poured slowly into an aqueous sodium acetate solution (10/a, 100 mL). The pH was adjusted to 4 with additional sodium acetate solution (10 mL) and extracted with ethyl acetate (20 mL×3). The combined organic phases were washed with brine (20 mL), dried over anhydrous sodium sulfate, filtered and concentrated to give (Z)-3-chloro-3-phenyl-prop-2-enal (2.50 g, 14.1 mmol, 56% yield) as a yellow oil. LCMS [M+1]-=167.1. ¹H NMR (400 MHz, CDCl₃) δ=10.24 (d, J=6.8 Hz, 1H), 7.49 (m, 5H), 6.69 (d, J=6.8 Hz, 1H).

Step 2: Hydrogen Cyanide (HCN) is produced as a byproduct in this reaction. Appropriate safety precaution and procedures should be employed. A mixture of (Z)-3-chloro-3-phenyl-prop-2-enal (1.76 g, 10.6 mmol, 1.00 eq.), ammonium thiocyanate (1.61 g, 21.1 mmol, 1.61 mL, 2.00 eq.) in acetone (25 mL) was degassed and purged with nitrogen and stirred at 80° C. for 1 hour. After such time the cooled mixture was poured into saturated sodium bicarbonate aqueous solution (200 mL) and extracted with ethyl acetate (100 mL×3). The combined organic phases were washed with brine (100 mL), dried over anhydrous sodium sulfate, filtered and concentrated. The formed residue was purified by column chromatography (SiO₂, petroleum ether/ethyl acetate 0-50%) to give 5-phenylisothiazole (1.00 g, 6.20 mmol, 58% yield) as a yellow oil. LCMS [M+1]⁺=162.2. ¹H NMR (400 MHz, CDCl₃) δ=8.49 (d, J=2.0 Hz, 1H), 7.64-7.60 (m, 2H), 7.48-7.43 (m, 3H), 7.42 (d, J=2.0 Hz, 1H).

Step 3: Bromine (952 mg, 5.95 mmol, 307 μL, 3.20 eq.) was added dropwise over a period of 30 min to a stirred mixture of 5-phenylisothiazole (300 mg, 1.86 mmol, 1.00 eq), potassium acetate (365 mg, 3.72 mmol, 2.00 eq.) and acetic acid (12 mL). The reaction mixture was stirred at 25° C. 5 hours and after such time treated with aqueous sodium bisulfite (33%, 10 mL). The solution was made basic with aqueous sodium hydroxide (20%, 10 mL), extracted with dichloromethane (3×80 mL). The combined organic extracts were dried (anhydrous sodium sulfate), filtered and concentrated to give to 4-bromo-5-phenyl-isothiazole, Intermediate AM (300 mg, 1.25 mmol, 67% yield) as colorless oil. LCMS [M+1]⁺=240.9. ¹H NMR (400 MHz, CDCl₃) δ=8.39 (s, 1H), 7.69-7.65 (m, 2H), 7.52-7.47 (m, 3H). ¹³C NMR (400 MHz, CDCl₃) δ=161.0, 159.5, 129.9, 129.3, 129.0, 128.5, 106.0.

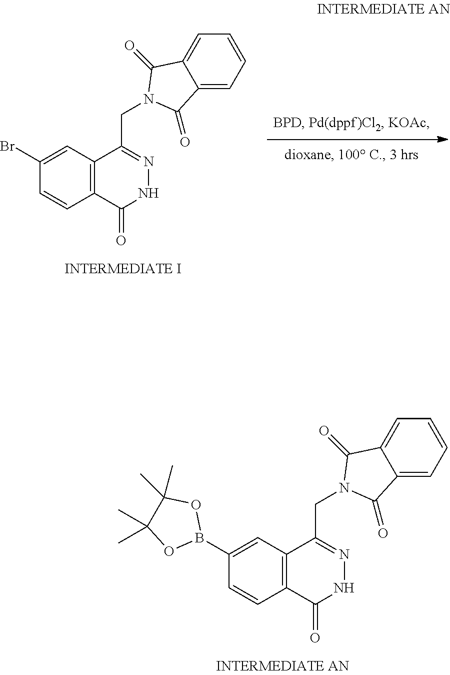 [SMILES: O=C1c2ccccc2C(=O)N1Cc1n[nH]c(=O)c2ccc(Br)cc12 ; CC1(C)OB(c2ccc3c(=O)[nH]nc(CN4C(=O)c5ccccc5C4=O)c3c2)OC1(C)C]

A mixture of intermediate I (160 g, 416 mmol, 1.00 eq.), 4,4,5,5-tetramethyl-2-(4,4,5,5-tetramethyl-1,3,2-dioxaborolan-2-yl)-1,3,2-dioxaborolane (BPD) (158 g, 624 mmol, 1.50 eq.), Pd(dppf)Cl₂ (30.4 g, 41.6 mmol, 0.10 eq.), potassium acetate (122 g, 1.25 mol, 3.00 eq.) in dioxane (2.0 L) was purged with nitrogen and stirred at 100° C. for 3 hrs. After such time the reaction mixture was filtered and concentrated under reduced pressure. The residue was triturated with MeOH (1.0 L) at 25° C. for 2 h, filtered and dried to give 2-((4-oxo-7-(4,4,5,5-tetramethyl-1,3,2-dioxaborolan-2-yl)-3,4-dihydrophthalazin-1-yl)methyl)isoindoline-1,3-dione, Intermediate AN (93.0 g, 209 mmol, 50% yield) as a gray solid. LCMS [M+1]⁺=432.4. ¹H NMR: (400 MHz DMSO-d₆) δ: 12.54 (s, 1H), 8.24-8.37 (m, 2H), 8.13 (d, J=7.6 Hz, 1H), 7.93-7.99 (m, 2H), 7.87-7.93 (m, 2H), 5.22 (s, 2H), 1.36 (s, 12H).

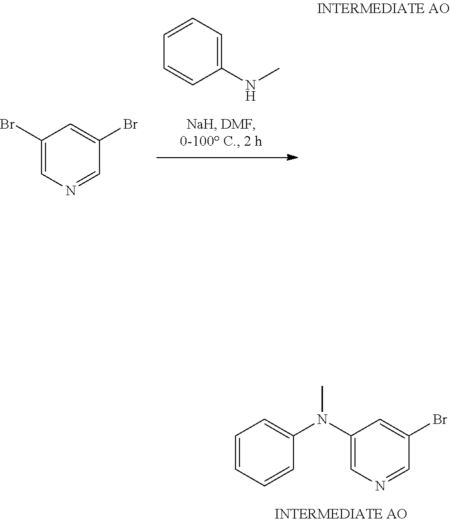

To a solution of 3,5-dibromopyridine (1.00 g, 4.22 mmol, 1.00 eq.) in DMF (10 mL) was added sodium hydride (270 mg, 6.75 mmol, 60% purity, 1.60 eq.) at 0° C. over 10 min followed by N-methylaniline (452 mg, 4.22 mmol, 458 μL, 1.00 eq.). The resulting mixture was stirred at 100° C. for 2 hours. After such time the reaction mixture was diluted with water (10 mL) and extracted with ethyl acetate (10 mL 3). The combined organic layers were washed with brine (50 mL×2), dried over anhydrous sodium sulfate, filtered and concentrated under reduced pressure. The residue was purified by flash silica gel chromatography (400 g SiO₂, water/acetonitrile, 0-100% 70 mL/min) to give 5-bromo-N-methyl-N-phenyl-pyridin-3-amine, Intermediate AO (50.0 mg, 190 μmol, 5% yield) as a yellow solid. LCMS [M+1]⁺262.9. ¹H NMR (400 MHz, CDCl₃-d) δ=8.12 (br d, J=6.4 Hz, 2H), 7.44-7.35 (m, 2H), 7.26-7.24 (m, 1H), 7.23-7.19 (m, 1H), 7.19-7.17 (m, 1H), 7.17-7.14 (m, 1H), 3.33 (s, 3H).

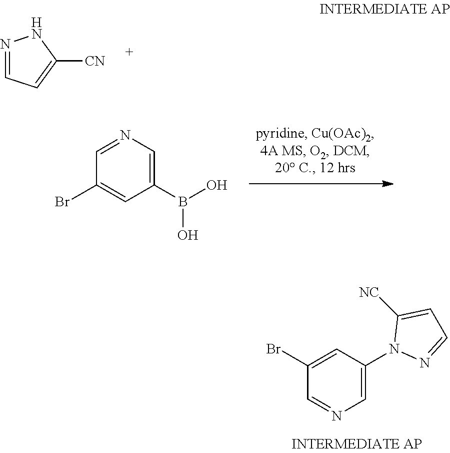 [SMILES: N#Cc1ccn[nH]1 ; OB(O)c1cncc(Br)c1 ; N#Cc1ccnn1-c1cncc(Br)c1]

A mixture of (5-bromo-3-pyridyl)boronic acid (325 mg, 1.61 mmol, 1.50 eq.), 1H-pyrazole-5-carbonitrile (100 mg, 1.07 mmol, 1.00 eq.), pyridine (255 mg, 3.22 mmol, 260 μL, 3.00 eq.), 4 Å molecular sieves (20.0 mg, 1.07 mmol) and copper acetate (585 mg, 3.22 mmol, 3.00 eq) in dichloromethane (5 mL) was degassed with nitrogen and stirred at 20° C. for 12 hours under an oxygen atmosphere (15 psi). After such time the reaction mixture was filtered and concentrated under reduced pressure and the residue was purified by column chromatography (SiO₂, petroleum ether:ethyl acetate 5-20%) to give 2-(5-bromo-3-pyridyl)pyrazole-3-carbonitrile, Intermediate AP (150 mg, 602 μmol, 56% yield) as a white solid. LCMS [M+1]249.0. ¹H NMR (400 MHz, DMSO-d₆) δ=9.15 (d, J=2.4 Hz, 1H), 8.91 (d, J=2.4 Hz, 1H), 8.78 (d, J=2.0 Hz, 1H), 8.63 (t, J=2.0 Hz, 1H), 7.34 (d, J=2.4 Hz, 1H).

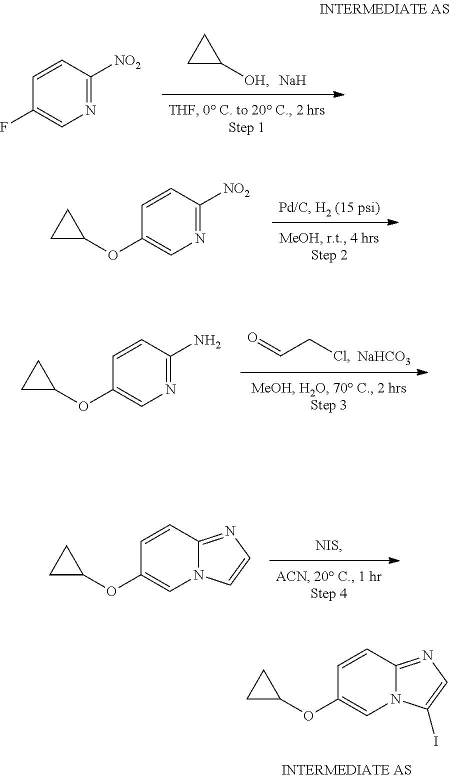

Step 1: To a solution of cyclopropanol (450 mg, 7.74 mmol, 1.10 eq.) in THF (10 mL) was added sodium hydride (310 mg, 7.74 mmol, 60.0% purity, 1.10 eq.) at 0° C., followed by 5-fluoro-2-nitro-pyridine (1.00 g, 7.04 mmol, 1.00 eq.) and the mixture was warmed to 20° C. and stirred for 2 hours. After completion, the mixture was filtered and concentrated in vacuum and the residue purified by column chromatography (SiO₂, petroleum ether:ethyl acetate 20-80%) to give 5-(cyclopropoxy)-2-nitro-pyridine (1.10 g, 6.11 mmol, 86% yield) as a white solid. LCMS [M+1]⁺=181.1.

Step 2: To a solution of 5-(cyclopropoxy)-2-nitro-pyridine (200 mg, 1.11 mmol, 1.00 eq.) in methyl alcohol (4 mL) was added palladium on activated carbon (100 mg, 1.11 mmol, 10% purity, 1.00 eq.) and the mixture was stirred at 30° C. for 4 hours under a hydrogen (15 psi) atmosphere. After completion, the reaction mixture was filtered, washed with methanol (5 mL×2) and concentrated to give 5-(cyclopropoxy)pyridin-2-amine (120 mg, 799 μmol, 72% yield) as a black oil which used for the next step without further purification. LCMS [M+1]⁺=151.1.

Step 3: To a solution of 5-(cyclopropoxy)pyridin-2-amine (120 mg, 799 μmol, 1.00 eq.) in methyl alcohol (2 mL) and water (1.0 mL) was added 2-chloroacetaldehyde (313 mg, 1.60 mmol, 257 μL, 2.00 eq.) and sodium bicarbonate (70.5 mg, 839 μmol, 1.05 eq.). The mixture was stirred at 70° C. for 2 hours. After such time the solvent was removed under reduced pressure, diluted with ethyl acetate (3 mL) and water (2 mL), and extracted with ethyl acetate (5 mL×3). The combined organic layers were washed with brine (10 mL×3), dried over anhydrous sodium sulfate, filtered and concentrated under reduced pressure to give 6-(cyclopropoxy)imidazo[1,2-a]pyridine (220 mg, crude) as a yellow solid which used for the next step without further purification. LCMS [M+1]⁺=175.2.

Step 4: To a solution of 6-(cyclopropoxy)imidazo[1,2-a]pyridine (220 mg, crude) in acetonitrile (2 mL) was added N-iodosuccinimide (313 mg, 1.39 mmol). The mixture was stirred at 20° C. for 1 hour. Upon completion the reaction mixture was concentrated under reduced pressure and the residue purified by column chromatography (SiO₂, petroleum ether:ethyl acetate 20-80%) to give 6-(cyclopropoxy)-3-iodo-imidazo[1,2-a]pyridine, Intermediate AS (220 mg, 733 μmol, 58% yield) as a white solid. LCMS [M+1]⁺=300.9. ¹H NMR (400 MHz, DMSO-d₆) δ=8.03 (d, J=2.0 Hz, 1H), 7.76 (s, 1H), 7.60 (d, J=9.6 Hz, 1H), 7.17 (dd, J=2.4, 9.6 Hz, 1H), 4.08-4.05 (m, 1H), 0.88-0.82 (m, 2H), 0.80-0.72 (m, 2H).

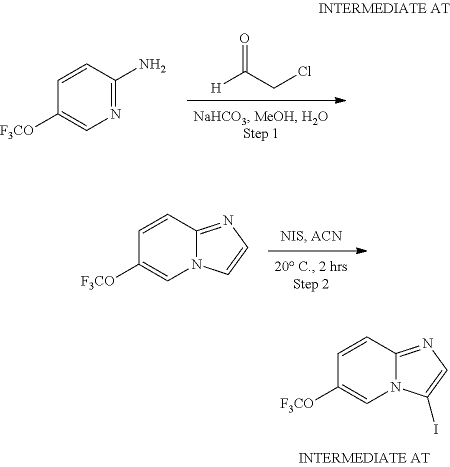

Step 1: To a solution of 5-(trifluoromethoxy)pyridin-2-amine (250 mg, 1.40 mmol, 1.00 eq.) in methanol (5 mL) and water (2.5 mL) was added 2-chloroacetaldehyde (289 mg, 1.47 mmol, 237 μL, 1.05 eq.) and sodium bicarbonate (118 mg, 1.41 mmol, 54.8 μL, 1.00 eq.). The mixture was stirred at 70° C. for 2 hours. The reaction mixture was then concentrated under reduced pressure and the residue diluted with water (20 mL) and extracted with ethyl acetate (20 mL×2). The combined organic layers were concentrated to give 6-(trifluoromethoxy)imidazo[1,2-a]pyridine (250 mg, crude) as a colorless oil which used for the next step without further purification.

Step 2: To a solution of 6-(trifluoromethoxy)imidazo[1,2-a]pyridine (238 mg, crude) in acetonitrile (10 mL) was added N-iodosuccinimide (291 mg, 1.30 mmol) in acetonitrile (5 mL) at 0° C., and the resulting yellow suspension was allowed to warm to 20° C. for 2 hours. The reaction mixture was then diluted with water (10 mL), extracted with ethyl acetate (10 mL×3) and the combined organic extracts were washed with brine (10 mL), dried, filtered and concentrated. The residue was purified by silica gel chromatography (petroleum ether:ethyl acetate 10-15%) to give 3-iodo-6-(trifluoromethoxy)imidazo[1,2-a]pyridine, Intermediate AT (180 mg, 548 μmol, 46% yield) as yellow solid. LCMS [M+1]⁺=329.0. ¹H NMR (400 MHz, CDCl₃) δ=8.23-8.19 (s, 1H), 7.80 (s, 1H), 7.71 (d, J=9.6 Hz, 1H), 7.27 (m, 1H).

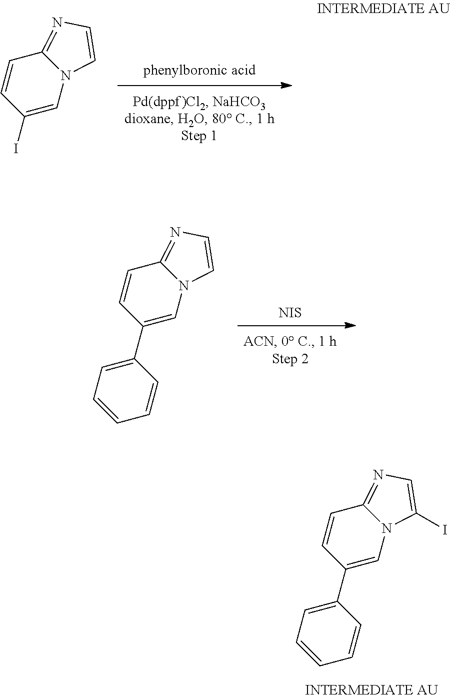 [SMILES: Ic1ccc2nccn2c1 ; "phenylboronic acid" ; c1ccc(-c2ccc3nccn3c2)cc1 ; "NIS" ; Ic1cnc2ccc(-c3ccccc3)cn12]

Step 1: A mixture of 6-iodoimidazo[1,2-a]pyridine (500 mg, 2.05 mmol, 1.00 eq.), phenylboronic acid (275 mg, 2.25 mmol, 1.10 eq.), Pd(dppf)Cl₃ (150 mg, 205 μmol, 0.10 eq.), sodium bicarbonate (344 mg, 4.10 mmol, 159 μL, 2.00 eq.) in dioxane (5 mL) and water (1.0 mL) was degassed with nitrogen and stirred at 80° C. for 1 hour. After such time the reaction mixture was concentrated under reduced pressure and the residue diluted with ethyl alcohol (5 mL) and the solution was then concentrated. The residue was purified by prep-TLC (dichloromethane:methyl alcohol, 10%) to give 6-phenylimidazo[1,2-a]pyridine (250 mg, 1.29 mmol, 62% yield, ) as a white solid. LCMS [M+1]⁺=195.1.

Step 2: To a solution of 6-phenylimidazo[1,2-a]pyridine (100 mg, 515 μmol, 1.00 eq.) in acetonitrile (2 mL) was added N-iodosuccinimide (127 mg, 566 μmol, 1.10 eq.) and the mixture was stirred at 0° C. for 1 hour. After such time the reaction mixture was concentrated under reduced pressure and the residue diluted with ethyl alcohol (2 mL) and the supernatant removed and concentrated under reduced pressure. The residue was purified by prep-TLC (petroleum ether:ethyl acetate 10%) to give 3-iodo-6-phenyl-imidazo[1,2-a]pyridine, Intermediate AU (120 mg, 375 μmol, 72% yield) as a yellow solid. LCMS [M+1]⁺=321.0. ¹H NMR (400 MHz, DMSO-d₆) δ=8.40 (br s, 1H), 7.77 (m, 3H), 7.73-7.62 (m, 2H), 7.58-7.49 (m, 2H), 7.45 (m, 1H).

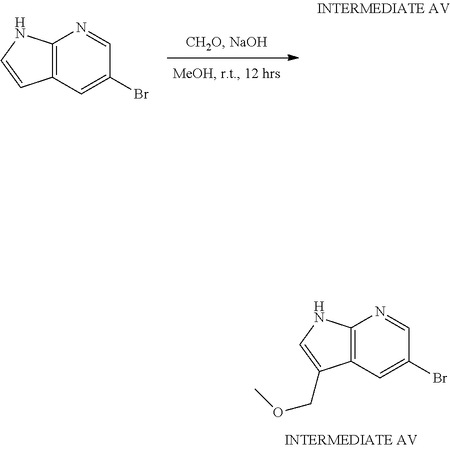

To a solution of 5-bromo-1H-pyrrolo[2,3-b]pyridine (2.00 g, 10.2 mmol, 1.00 eq.) in methyl alcohol (10 mL) was added formaldehyde (610 mg, 20.3 mmol, 559 μL, 2.00 eq.) and sodium hydroxide (812 mg, 20.3 mmol, 2.00 eq.) and the mixture was stirred at 20° C. for 2 hours. After completion the reaction mixture was then filtered and concentrated under vacuum. The residue was purified by silica gel column chromatography (petroleum ether:ethyl acetate 5-20%) then by prep-TLC (petroleum ether:ethyl acetate 20%) to give 5-bromo-3-(methoxymethyl)-1H-pyrrolo[2,3-b]pyridine, Intermediate AV (120 mg, 498 μmol, 5% yield) as white solid. LCMS [M+1]⁺=243.0. ¹H NMR (400 MHz, DMSO-d₆) δ=11.83 (br s, 1H), 8.28 (d, J=2.4 Hz, 1H), 8.18 (d, J=2.0 Hz, 1H), 7.56 (d, J=2.4 Hz, 1H), 4.53 (s, 2H), 3.25 (s, 3H).

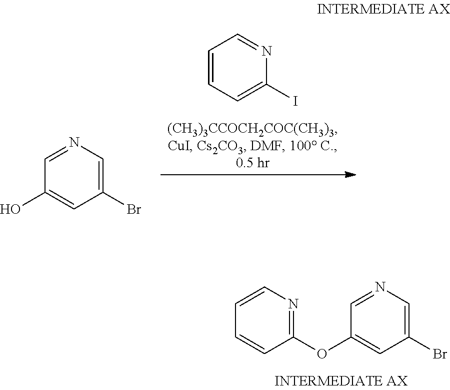 [SMILES: Ic1ccccn1 ; Oc1cncc(Br)c1 ; "(CH3)3CCOCH2COC(CH3)3,CuI, Cs2CO3, DMF, 100° C.,0.5 hr" ; Brc1cncc(Oc2ccccn2)c1]

To a solution of 5-bromopyridin-3-ol (500 mg, 2.87 mmol, 1.00 eq.) in DMF (10 mL) was added cesium carbonate (1.87 g, 5.75 mmol, 2.00 eq.), 2-iodopyridine (707 mg, 3.45 mmol, 366 μL, 1.20 eq.), 2,2,6,6-tetramethylheptane-3,5-dione (212 mg, 1.15 mmol, 237 μL, 0.40 eq.) and cuprous iodide (109 mg, 575 μmol, 0.20 eq.). The mixture was stirred at 100° C. for 0.5 hour. The reaction mixture was then diluted with water (100 mL) and extracted with ethyl acetate (70.0 mL×3). The combined organic layers were washed with brine (100 mL), dried, filtered and concentrated under reduced pressure. The residue was purified by column chromatography (SiO₂, petroleum ether:ethyl acetate 0-20%) to give 3-bromo-5-(2-pyridyloxy)pyridine Intermediate AX (600 mg, 1.45 mmol, 50% yield) as a yellow oil. LCMS [M+1]⁺=250.8. ¹H NMR (400 MHz, DMSO-d₆) δ=8.57 (d, J=2.0 Hz, 1H), 8.48 (d, J=2.4 Hz, 1H), 8.16 (ddd, J=0.8, 2.0, 4.8 Hz, 1H), 8.02 (s, 1H), 7.91 (ddd, J=2.0, 7.2, 8.0 Hz, 1H), 7.21-7.15 (m, 2H).

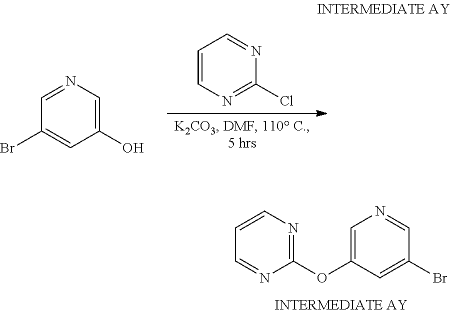

To a solution of 2-chloropyrimidine (300 mg, 2.62 mmol, 1.00 eq.) in DMF (2 mL) was added potassium carbonate (724 mg, 5.24 mmol, 2.00 eq.) and 5-bromopyridin-3-ol (479 mg, 2.75 mmol, 1.05 eq.). The mixture was stirred at 110° C. for 5 hours. The reaction mixture was diluted with water (200 mL) and extracted with ethyl acetate (80 mL×3). The combined organic layers were washed with brine (150 mL), dried over anhydrous sodium sulfate, filtered and concentrated under reduced pressure to give 2-[(5-bromo-3-pyridyl)oxy]pyrimidine, Intermediate AY (523 mg, crude) as a red solid and used into the next step directly without further purification. LCMS [M+1]⁺=252.0; ¹H NMR (400 MHz, DMSO-d₆) δ=8.70 (s, 1H), 8.69 (s, 1H), 8.63 (d, J=2.0 Hz, 1H), 8.57 (d, J=2.4 Hz, 1H), 8.17 (t, J=2.0 Hz, 1H), 7.34 (t, J=4.8 Hz, 1H).

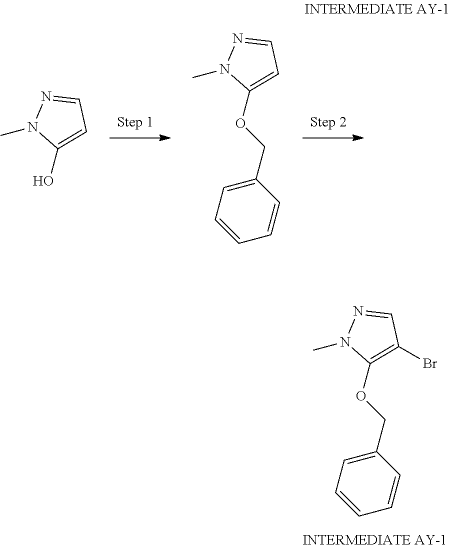

Step 1: To a solution of 2-methylpyrazol-3-ol (500 mg, 5.10 mmol, 1.00 eq.), (bromomethyl)benzene (1.05 g, 6.12 mmol, 726 μL, 1.20 eq.) in DMF (6.0 mL) was added potassium carbonate (1.06 g, 7.65 mmol, 1.50 eq.). The mixture was stirred at 120° C. for 4 hours. The reaction mixture was then diluted with water (10 mL) and extracted with ethyl acetate (20 mL×3). The combined organic layers were washed with brine (50 mL×2), dried over anhydrous sodium sulfate, filtered and concentrated under reduced pressure. The residue was purified by column chromatography (SiO₂, petroleum ether/ethyl acetate 50/1 to 6/1) to give 5-benzyloxy-1-methyl-pyrazole (450 mg, 2.39 mmol, 47% yield) as a colorless oil. LCMS [M+1]⁺=189.2; ¹H NMR (400 MHz, CDCl₃) δ=7.43-7.41 (m, 3H), 7.40-7.35 (m, 2H), 7.31 (d, J=2.0 Hz, 1H), 7.24-7.21 (d, J=2.0 Hz, 1H), 5.08 (s, 2H), 3.67 (s, 3H).

Step 2: To a solution of 5-benzyloxy-1-methyl-pyrazole (400 mg, 2.13 mmol, 1.00 eq.) in acetonitrile (6 mL) was added NBS (416 mg, 2.34 mmol, 1.10 eq.). The mixture was stirred at 0° C. for 0.5 hours. The reaction mixture was then concentrated under reduced pressure and the residue was purified by column chromatography (SiO₂, petroleum ether/ethyl acetate=100/1 to 20/1) to give 5-benzyloxy-4-bromo-1-methyl-pyrazole (320 mg, 1.20 mmol, 56% yield) as a yellow oil. LCMS [M+1]⁺=266.9; ¹H NMR (400 MHz, CDCl₃) δ=7.39 (s, 5H), 7.32 (s, 1H), 5.28 (s, 2H), 3.45 (s, 3H).

Intermediate AY-2 shown in Table I-IIb was prepared following the teachings of the General Reaction Schemes and the method to prepare Intermediate AY-1.

TABLE I-IIb

| Intermediate | Structure | Spectral Data |
|---|---|---|
| AY-2 | 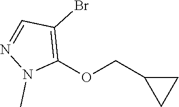 | 4-bromo-5-(cyclopropylmethoxy)-1-methyl-1H-pyrazole LCMS [M + 1]⁺ = 233.1; ¹H NMR (400 MHz, CDCl₃) δ = 7.32-7.28 (s, 1H), 4.11-4.07 (m, 2H), 3.74-3.68 (s, 3H), 1.25-1.16 (m, 1H), 0.67-0.57 (m, 2H), 0.37-0.28 (m, 2H) |

### General Procedure for Intermediates B-1 to B-15

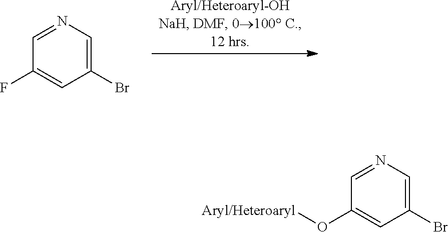

To the corresponding aryl/heteroaryl phenol (3.89 mmol, 1.00 eq.) in DMF (10 mL) was added sodium hydride (4.28 mmol, 60% purity, 1.10 eq.) at 0° C. under nitrogen. After the addition was complete the mixture was stirred at 25° C. for 0.5 hour, followed by addition with 3-bromo-5-fluoro-pyridine (3.89 mmol, 1.00 eq.) and stirred at 100° C. for a further 12 hours. After such time the reaction mixture was quenched by the addition water (10 mL) and then extracted with ethyl acetate (20 mL 3). The combined organic layers were washed with brine (50 mL×2), dried over anhydrous sodium sulfate, filtered and concentrated under reduced pressure to give a residue which was used directly in the next step without further purification.

Following the teachings of the General Reaction Schemes and the general procedure for Intermediates B-1 to B-0 the intermediates in Table I-III were prepared.

TABLE I-III

| Intermediate | Structure | Characterization |
|---|---|---|
| B-1 | 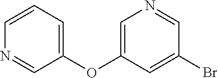 [SMILES: Brc1cncc(Oc2cccnc2)c1] | 3-Bromo-5-(pyridin-3-yloxy )pyridine LCMS [M + 1]⁺ = 250.9; ¹H NMR (400 MHz, CDCl₃) δ = 8.51-8.48 (m, 2H), 8.48-8.45 (m, 1H), 8.36 (d, J = 2.8 Hz, 1H), 7.47(t, J = 2.0 Hz 1H), 7.40-7.35 (m, 2H) |
| B-2 | 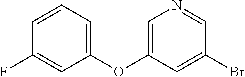 [SMILES: Fc1cccc(Oc2cncc(Br)c2)c1] | 3-Bromo-5-(3-fluorophenoxy)pyridine LCMS [M + 1]⁺ = 268.0; ¹H NMR (400 MHz, DMSO-d₆) δ = 8.53 (d, J = 1.6 Hz, 1H), 8.41 (d, J = 2.4 Hz, 1H), 7.81 (t, J = 2.0 Hz, 1H), 7.51-7.43 (m, 1H), 7.08-7.03 (m, 2H), 6.97-6.92 (m, 1H) |
| B-3 | 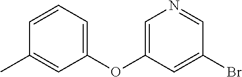 | 3-Bromo-5-(m-tolyloxy)pyridine LCMS [M + 1]⁺ = 265.9; ¹H NMR (400 MHz, CDCl₃) δ = 8.40 (d, J = 2.0 Hz, 1H), 8.33 (d, J = 2.4 Hz, 1H), 7.41 (t, J = 2.0 Hz, 1H), 7.31-7.27 (m, 1H), 7.03 (d, J = 7.6 Hz, 1H), 6.92-6.81 (m, 2H), 2.37 (s, 3H) |
| B-4 | 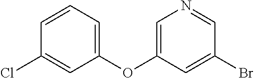 [SMILES: Clc1cccc(Oc2cncc(Br)c2)c1] | 3-Bromo-5-(3-chlorophenoxy)pyridine LCMS [M + 1]⁺ = 286.0; ¹H NMR (400 MHz, DMSO-d₆) δ = 8.53 (d, J = 1.6 Hz, 1H), 8.41 (d, J = 2.4 Hz, 1H), 7.82 (q, J = 2.4 Hz, 1H), 7.49- 7.41 (m, 1H), 7.31-7.23 (m, 2H), 7.11-7.06 (m, 1H) |
| B-5 | 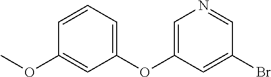 [SMILES: COc1cccc(Oc2cncc(Br)c2)c1] | 3-Bromo-5-(3-methoxyphenoxy)pyridine LCMS [M + 1]⁺ = 279.9; ¹H NMR (400 MHz, CDCl₃) δ = 8.42 (d, J = 2.0 Hz, 1H), 8.34 (d, J = 2.4 Hz, 1H), 7.44 (t, J = 2.4 Hz, 1H), 7.30 (t, J = 8.4 Hz, 1H), 6.79-6.74 (m, 1H), 6.64-6.59 (m, 2H), 3.81 (s, 3H) |
| B-6 | 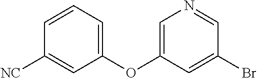 [SMILES: N#Cc1cccc(Oc2cncc(Br)c2)c1] | 3-((5-Bromopyridin-3-y1)oxy)benzonitrile LCMS [M + 1]⁺ = 277.0; ¹H NMR (400 MHz, DMSO-d₆) δ = 8.55 (d. J = 1.6 Hz, 1H), 8.43 (d, J = 2.4 Hz, 1H), 7.86 (t, J = 2.4 Hz, 1H), 7.69-7.66 (m, 2H), 7.62 (t, J = 8.4 Hz, 1H), 7.47 (ddd, J = 1.2, 2.4, 8.0 Hz, 1H) |
| B-7 | 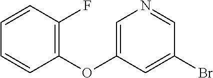 [SMILES: Fc1ccccc1Oc1cncc(Br)c1] | 3-Bromo-5-(2-fluorophenoxy)pyridine LCMS [M + 1]⁺ = 269.9; ¹H NMR (400 MHz, DMSO-d₆) δ = 8.50 (d, J = 1.6 Hz, 1H), 8.38 (d, J = 2.4 Hz, 1H), 7.70 (t, J = 2.0 Hz, 1H), 7.48-7.39 (m, 1H), 7.36-7.27 (m, 3H) |
| B-8 | 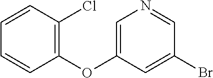 [SMILES: Clc1ccccc1Oc1cncc(Br)c1] | 3-Bromo-5-(2-chlorophenoxy)pyridine LCMS [M + 1]⁺ = 286.0; ¹H NMR (400 MHz, DMSO-d₆) δ = 8.49 (d. J = 1.6 Hz, 1H), 8.33 (d, J = 2.4 Hz, 1H), 7.66-7.62 (m, 2H), 7.43 (td, J = 1.6, 8.0 Hz, 1H), 7.34-7.28 (m, 2H) |
| B-9 | 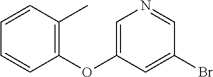 | 3-Bromo-5-(o-tolyloxy)pyridine LCMS [M + 1]⁺ = 266.0; ¹H NMR (400 MHz, CDCl₃) δ = 8.37 (d, J = 2.0 Hz, 1H), 8.28 (d, J = 2.4 Hz, 1H), 7.31 (dd, J = 0.8, 7.2 Hz, 1H), 7.29 (s, 1H), 7.27-7.22 (td, J = 1.6,7.6 Hz, 1H), 7.20-7.14 (td, J = 1.2, 7.6 Hz, 1H), 6.96 (dd, J = 1.2, 8.0 Hz, 1H), 2.23 (s, 3H) |
| B-10 | 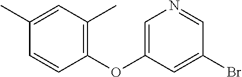 [SMILES: Cc1ccc(Oc2cncc(Br)c2)c(C)c1] | 3-Bromo-5-(2,4-dimethylphenoxy)pyridine LCMS [M + 1]⁺ = 278.0; ¹H NMR (400 MHz, CDCl₃) δ = 8.35 (d, J = 2.0 Hz, 1H), 8.26 (d, J = 2.4 Hz, 1H), 7.26-7.24 (t, J = 2.4 Hz, 1H), 7.10 (s, 1H), 7.04 (dd, J = 1.2, 8.4 Hz, 1H), 6.86 (d, J = 8.4 Hz, 1H), 2.35 (s, 3H), 2.17 (s, 3H) |
| B-11 | 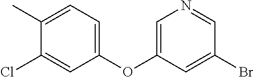 [SMILES: Cc1ccc(Oc2cncc(Br)c2)cc1Cl] | 3-Bromo-5-(3-chloro-4-methylphenoxy)pyridine LCMS [M + 1]⁺ = 299.9; ¹H NMR (400 MHz, CDCl₃) δ = 8.43 (d, J = 2.0 Hz, 1H), 8.32 (d, J = 2.4 Hz, 1H), 7.44-7.41 (t, J = 2.4, 1H), 7.26 (d, J = 8.8 Hz, 1H), 7.08 (d, J = 2.4 Hz, 1H), 6.87 (dd, J = 1.6, 4.4 Hz, 1H), 2.38 (s, 3H). |
| B-12 | 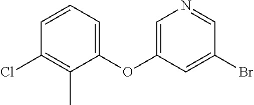 [SMILES: Cc1c(Cl)cccc1Oc1cncc(Br)c1] | 3-Bromo-5-(3-chloro-2-methylphenoxy)pyridine LCMS [M + 1]⁺ = 299.9; ¹H NMR (400 MHz, CDCl₃) δ = 8.41 (d, J = 2.0 Hz, 1H), 8.28 (d, J = 2.4 Hz, 1H), 7.32-7,31 (t, J = 2.0 Hz, 1H), 7.17 (t, J = 8.0 Hz, 1H), 6.88 (dd, J = 0.8, 8.0 Hz, 1H), 6.69 (dd, J = 1,6, 7.6 Hz, 1H), 2.31 (s, 3H) |
| B-13 | 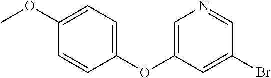 [SMILES: COc1ccc(Oc2cncc(Br)c2)cc1] | 3-Bromo-5-(4-methoxyphenoxy)pyridine LCMS [M + 1]⁺ = 280.1; ¹H NMR (400 MHz, CDCl₃) δ = 8.36 (d, J = 2.0 Hz, 1H), 8.29 (d, J = 2.4 Hz, 1H), 7.35-7.33 (m, 1H), 7.04-6.99 (m, 2H), 6.96-6.91 (m, 2H), 3.84 (s, 3H) |
| B-14 | 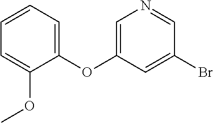 [SMILES: COc1ccccc1Oc1cncc(Br)c1] | 3-Bromo-5-(2-methoxyphenoxy)pyridine LCMS [M + 1]⁺ = 280.1; ¹H NMR (400 MHz, CDCl₃) δ = 8.35 (d, J = 1.6 Hz, 1H), 8.27 (d, J = 2.4 Hz, 1H), 7.29 (dd, J = 2.0, 2.4 Hz, 1H), 7.08- 7.01 (m, 2H), 6.95-6.86 (m, 2H), 3.82 (s, 3H) |
| B-15 | 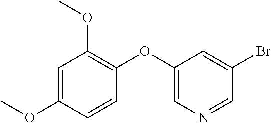 [SMILES: COc1ccc(Oc2cncc(Br)c2)c(OC)c1] | 3-bromo-5-(2,4-dimethoxyphenoxy)pyridine LCMS [M + 1]⁺ = 312.0; ¹H NMR (400 MHz, CDCl₃) δ = 8.32 (d, J = 1.6 Hz, 1H), 8.25 (d, J = 2.4 Hz, 1H), 7.24 (t, J = 2.0 Hz, 1H), 7.02 (d, J = 8.8 Hz, 1H), 6.60 (d, J = 2.8 Hz, 1H), 6.49 (dd, J = 2.8, 8.8 Hz, 1H), 3.84 (s, 3H), 3.78 (s, 3H) |

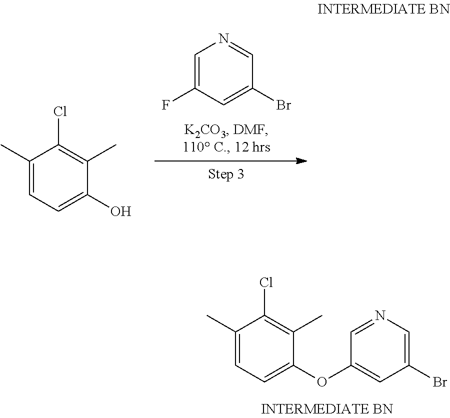

To a solution of 3-bromo-5-fluoro-pyridine (210 mg, 1.19 mmol, 0.95 eq.) in DMF (10 mL) was added potassium carbonate (347 mg, 2.51 mmol, 2.00 eq.) and 3-chloro-2, 4-dimethyl-phenol (197 mg, 1.26 mmol, 1.00 eq.). The mixture was stirred at 110° C. for 12 hours. After such time the reaction mixture was diluted with water (80 mL) and extracted with ethyl acetate (50 mL×3). The combined organic layers were then washed with brine (50 mL), dried over anhydrous sodium sulfate, filtered and concentrated under reduced pressure. The residue was purified by column chromatography (SiO₂, Petroleum ether:ethyl acetate 0-20%) to give 3-bromo-5-(3-chloro-2, 4-dimethyl-phenoxy) pyridine, Intermediate BN (178 mg, 569 μmol, 45% yield) as a colorless oil. LCMS [M+1]⁺=314.0. ¹H NMR (400 MHz, DMSO-d₆) δ=8.45 (d, J=2.0 Hz, 1H), 8.31 (d, J=2.4 Hz, 1H), 7.57 (t, J=2.0 Hz, 1H), 7.27 (d, J=8.4 Hz, 1H), 6.99 (d, J=8.4 Hz, 1H), 2.34 (s, 3H), 2.22 (s, 3H).

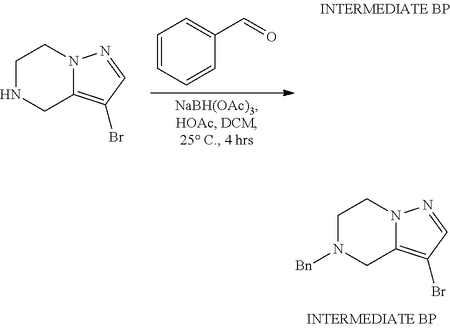

To a solution of 3-bromo-4,5,6,7-tetrahydropyrazolo[1,5-a]pyrazine (300 mg, 949 μmol, 1.00 eq., TFA) and benzaldehyde (131 mg, 1.23 mmol, 125 μL, 1.30 eq.) in dichloromethane (10 mL) was added sodium triacetoxyborohydride (402 mg, 1.90 mmol, 2.00 eq.) and acetic acid (114 mg, 1.90 mmol, 109 μL, 2.00 eq.). The mixture was then stirred at 25° C. for 4 hours. After such time the reaction mixture was diluted with water (10 mL) and extracted with ethyl acetate (15 mL×3). The combined organic layers were washed with brine (50 mL×2), dried over anhydrous sodium sulfate, filtered and concentrated. The residue was purified by prep-TLC (SiO₂, petroleum ether:ethyl acetate 25%) to give 5-benzyl-3-bromo-6,7-dihydro-4H-pyrazolo[1,5-a]pyrazine, Intermediate BP (170 mg, 582 μmol, 61% yield) as a colorless oil. LCMS [M+1]⁺=294.0. ¹H NMR (400 MHz, CD₃OD) δ=7.45 (s, 1H), 7.41-7.32 (m, 5H), 4.12 (t, J=5.6 Hz, 2H), 3.77 (s, 2H), 3.57 (s, 2H), 2.96 (t, J=5.6 Hz, 2H).

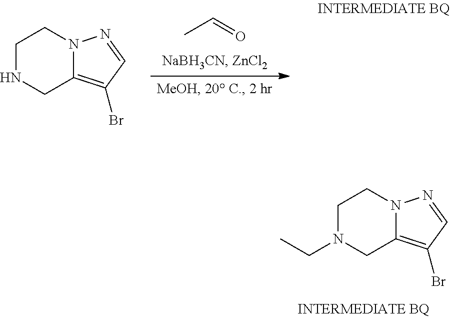

A mixture of 3-bromo-4,5,6,7-tetrahydropyrazolo[1,5-a]pyrazine (400 mg, 1.27 mmol, 1.00 eq.), acetaldehyde (5.0 M, 508 μL, 2.01 eq.), sodium cyanoborohydride (160 mg, 2.54 mmol, 2.01 eq.), zinc chloride (1.0 M, 2.53 mL, 2.00 eq.) in methanol (8 mL) was stirred at 25° C. for 2 hours. After such time the solvent was evaporated and the residue purified by column chromatography (SiO₂, dichloromethane:methanol 0-10%). The product was further purified by prep-HPLC (Waters Xbridge C18 150×50 mm×10 μm; mobile phase: [water (10 mM NH₄HCO₃)-ACN]; B %: 16%-46%, 11.5 min) to give 3-bromo-5-ethyl-6,7-dihydro-4H-pyrazolo[1,5-a]pyrazine, intermediate BQ (100 mg, 434 μmol, 34% yield) as an colorless oil. ¹H NMR (400 MHz, CD₃OD) δ=7.47 (s, 1H), 4.21-4.11 (t, J=6.0 Hz, 2H), 3.61 (s, 2H), 3.03-2.94 (t, J=6.0 Hz, 2H), 2.69 (q, J=7.2 Hz, 2H), 1.20 (t, J=7.2 Hz, 3H).

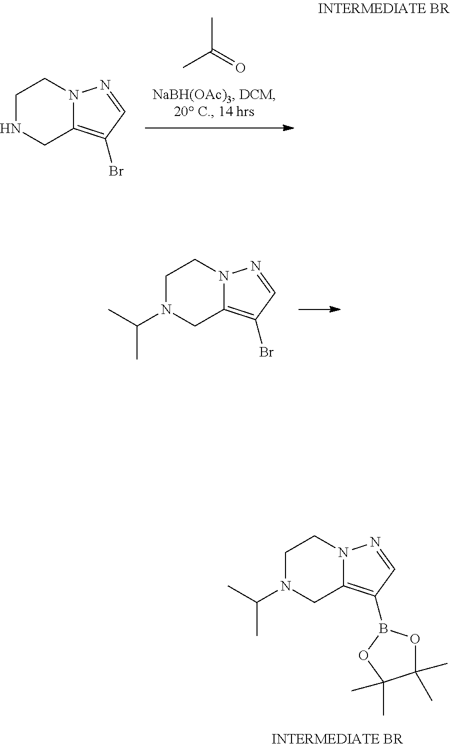

Step 1: To a mixture of 3-bromo-4,5,6,7-tetrahydropyrazolo[1,5-a]pyrazine (200 mg, 990 μmol, 1.00 eq.) and acetone (862 mg, 14.9 mmol, 1.09 mL, 15.0 eq.) in dichloromethane (1.0 mL), was added sodium triacetoxyborohydride (420 mg, 1.98 mmol, 2.00 eq.). After stirring at 25° C. for 14 hours the mixture was extracted with dichloromethane (5 mL×3), washed with brine, dried over sodium sulfate, filtered and concentrated under reduced pressure. The residue was purified by prep-TLC (SiO₂, dichloromethane:methyl alcohol 10%) to give 3-bromo-5-isopropyl-6,7-dihydro-4H-pyrazolo[1,5-a]pyrazine (150 mg, 531 μmol, 54% yield) as a yellow oil. LCMS [M+1]⁺=244.0. ¹H NMR (400 MHz, CDCl₃) δ=7.35 (s, 1H), 4.12-4.06 (t, J=5.2 Hz, 2H), 3.59 (s, 2H), 2.96-2.90 (m, 1H), 2.89-2.86 (t, J=5.2 Hz, 2H), 1.09 (s, 3H), 1.07 (s, 3H).

Step 2: A mixture of 3-bromo-5-isopropyl-6,7-dihydro-4H-pyrazolo[1,5-a]pyrazine (80.0 mg, 328 μmol, 1.00 eq.), 4,4,4′,4′,5,5,5′,5′-octamethyl-2,2′-bi(1,3,2-dioxaborolane) (166 mg, 655 μmol, 2.00 eq.), potassium acetate (113 mg, 1.15 mmol, 3.50 eq.) and PdCl₂[P(Cy)₃]₂ (24.2 mg, 32.8 μmol, 0.10 eq.) in dimethylaminopyridine (1 mL) was purged with nitrogen then stirred at 90° C. for 20 hours. The mixture was then concentrated under reduced pressure to give 5-isopropyl-3-(4,4,5,5-tetramethyl-1,3,2-dioxaborolan-2-yl)-6,7-dihydro-4H-pyrazolo[1,5-a]pyrazine, Intermediate BR (700 mg, 303 μmol, 92% yield) as a black solid. LCMS [M+1]=292.2.

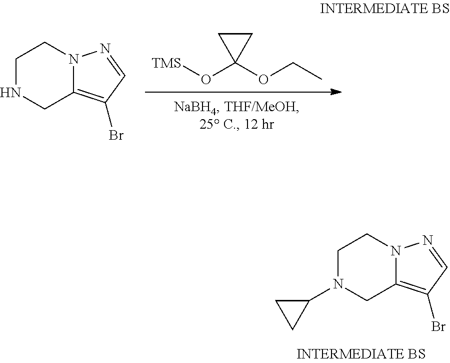

A pressure tube was charged with 3-bromo-4,5,6,7-tetrahydropyrazolo[1,5-a]pyrazine (260 mg, 1.29 mmol, 1.00 eq.), (1-ethoxycyclopropoxy)trimethylsilane (673 mg, 3.86 mmol, 776 μL, 3.00 eq.), sodium cyanoborohydride (243 mg, 3.86 mmol, 3.00 eq.) and acetic acid (773 mg, 12.9 mmol, 736 μL, 10.0 eq.) in THF (5 mL) and ethyl alcohol (5 mL). The resulting solution was stirred for 2 hours at 60° C., then the reaction mixture was concentrated and the residue was purified by prep-TLC (SiO₂, petroleum ether:ethyl acetate 20%) to give 3-bromo-5-cyclopropyl-6,7-dihydro-4H-pyrazolo[1,5-a]pyrazine, Intermediate BS (150 mg, 620 μmol, 48% yield) as yellow solid. ¹H NMR (400 MHz, CD₃OD) δ=7.45 (s, 1H), 4.14-4.09 (m, 2H), 3.75 (s, 2H), 3.20-3.10 (m, 2H), 2.02-1.96 (m, 1H), 0.65-0.57 (m, 2H), 0.56-0.44 (m, 2H).

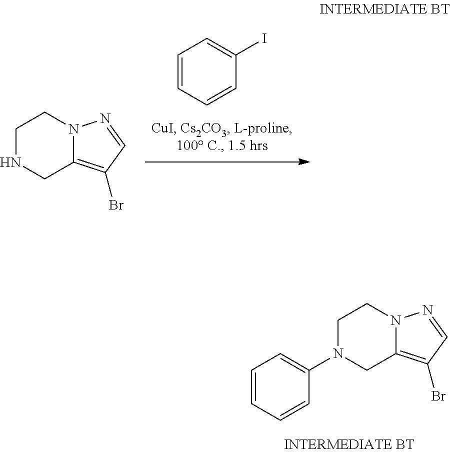 [SMILES: Ic1ccccc1 ; Brc1cnn2c1CNCC2 ; Brc1cnn2c1CN(c1ccccc1)CC2]

A mixture of 3-bromo-4,5,6,7-tetrahydropyrazolo[1,5-a]pyrazine (500 mg, 1.58 mmol, 1.00 eq.), iodobenzene (1.29 g, 6.33 mmol, 705 μL, 4.00 eq.), copper iodide (60.3 mg, 316 μmol, 0.20 eq.), (2S)-pyrrolidine-2-carboxylic acid (72.9 mg, 633 μmol, 0.40 eq.) and cesium carbonate (1.03 g, 3.16 mmol, 2.00 eq.) in DMF (10 mL) was degassed and purged with nitrogen then stirred at 100° C. for 1.5 hours. After such time the mixture was cooled, extracted with ethyl acetate (5 mL×3), washed with brine, dried over sodium sulfate, filtered and concentrated under reduced pressure. The formed residue was purified by column chromatography (SiO₂, petroleum ether/ethyl acetate 5-10%) to give 3-bromo-5-phenyl-6,7-dihydro-4H-pyrazolo[1,5-a]pyrazine, Intermediate BT (55 mg, 197 μmol, 12% yield) as yellow solid. LCMS [M+1]⁺=278.2.

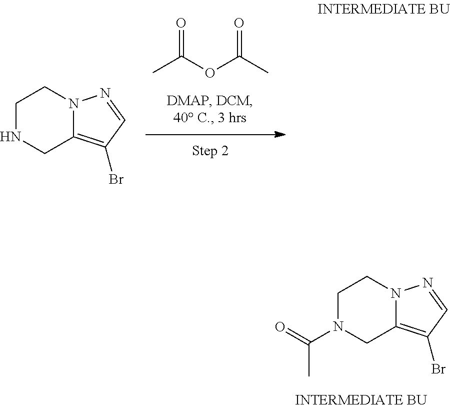

A mixture of 3-bromo-4,5,6,7-tetrahydropyrazolo[1,5-a]pyrazine (200 mg, 633 μmol, 1.00 eq.), acetyl acetate (96.9 mg, 949 μmol, 88.9 μL, 1.50 eq.) and DMAP (7.73 mg, 63.3 μmol, 0.10 eq.) in dichloromethane (10 mL) was degassed and purged with nitrogen then stirred at 40° C. for 3 hours. Upon completion the reaction mixture was concentrated under reduced pressure to give 1-(3-bromo-6,7-dihydro-4H-pyrazolo[1,5-a]pyrazin-5-yl)ethenone, Intermediate BU (100 mg, crude) as a white solid. LCMS [M+1]⁺=244.2.

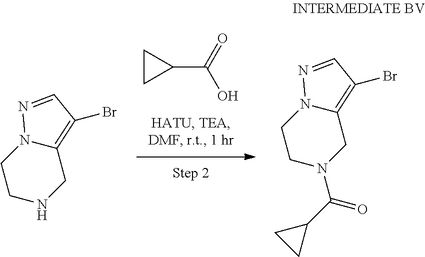

To a solution of 33-bromo-4,5,6,7-tetrahydropyrazolo[1,5-a]pyrazine (250 mg, 791 mol, 1.00 eq.) in dimethylformamide (2 mL) was added triethylamine (240 mg, 2.37 mmol, 330 μL, 3.00 eq.), HATU (601 mg, 1.58 mmol, 2.00 eq.), and cyclopropane carboxylic acid (102 mg 1.19 mmol, 93.7 BL, 1.50 eq.). The mixture was stirred at 35 (C for 1 hour. After such time the reaction mixture was diluted with water (100 mL) and extracted with ethyl acetate (30 mL 3) and the combined organic layers were washed with brine (50 mL), dried over anhydrous sodium sulfate, filtered and concentrated under reduced pressure. The formed residue was purified by column chromatography (SiO₂, petroleum ether:ethyl acetate 0-50%) to give (3-bromo-6,7-dihydro-4H-pyrazolo[1,5-a]pyrazin-5-yl)-cyclopropyl-methanone, Intermediate BV (139 mg, 515 μmol, 65 yield) as a colorless oil. ¹H NMR (400 MHz, CDCl₃) δ=7.48 (s, 1H), 4.89-4.68 (m, 2H), 4.33-4.05 (m, 4H), 1.87-1.79 (m, 1H), 1.09-1.04 (m, 2H), 0.93-0.84 (m, 2H).

Following the teachings of the General Reaction Schemes, and the procedure for INTERMEDIATE BV, INTERMEDIATES C-1 to C-5 were prepared as shown in Table I-IV:

TABLE I-IV

| Intermediate | Structure | Characterization |
|---|---|---|
| C-1 | 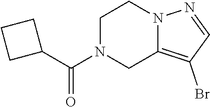 [SMILES: O=C(C1CCC1)N1CCn2ncc(Br)c2C1] | (3-Bromo-6,7-dihydropyrazolo[1,5-a]pyrazin-5(4H)- yl)(cyclobutyl)methanone LCMS [M + 1]⁺ = 286.1 ¹H NMR (400 MHz, DMSO-d₆) δ = 7.59 (s, 1H), 4.60-4.49 (m, 2H), 4.14-4.05 (m, 2H), 3.96-3.79 (m, 2H), 3.55-3.47 (m, 1H), 2.25-2.10 (m, 4H), 1.98-1.88 (m, 1H), 1.81-1.70 (m, 1H) |
| C-2 | 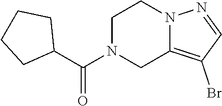 [SMILES: O=C(C1CCCC1)N1CCn2ncc(Br)c2C1] | (3-Bromo-6,7-dihydropyrazolo[1,5-a]pyrazin-5(4H)- y1)(cyclopenty1)methanone LCMS [M + 1]⁺ = 297.9 |
| C-3 | 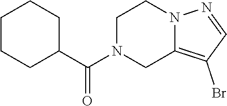 [SMILES: O=C(C1CCCCC1)N1CCn2ncc(Br)c2C1] | (3-Bromo-6,7-dihydropyrazolo[1,5-a]pyrazin-5(4H)- y1)(cyclohexyl)methanone ¹H NMR (400 MHz, DMSO-d₆) δ = 7.59 (s, 1H), 4.78-4.50 (m, 2H), 4.16 (m, 1H), 4.06-3.91 (m, 3H), 2.79-2.70 (m, 1H), 1.77-1.58 (m, 5H), 1.44-1.17 (m, 5H) |
| C-4 | 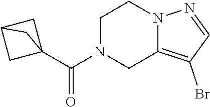 [SMILES: O=C(N1CCn2ncc(Br)c2C1)C12CC(C1)C2] | bicyclo[1.1.1]pentan-1-yl(3-bromo-6,7-dihydropyrazolo[1,5-a]pyrazin- 5(4H)-y1)methanone LCMS [M + 1]⁺ = 298.1 |
| C-5 | 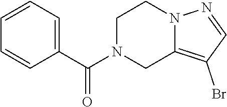 [SMILES: O=C(c1ccccc1)N1CCn2ncc(Br)c2C1] | (3-Bromo-6,7-dihydropyrazolo[1,5-a]pyrazin-5(4H)- y1)(phenyl)methanone LCMS [M + 1]⁺ = 308.0 |

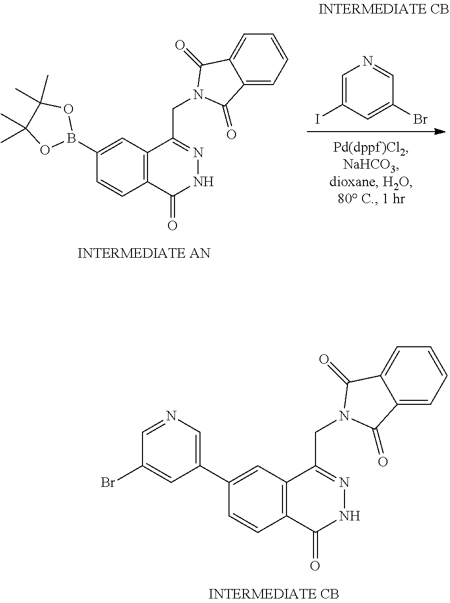 [SMILES: CC1(C)OB(c2ccc3c(=O)[nH]nc(CN4C(=O)c5ccccc5C4=O)c3c2)OC1(C)C ; Brc1cncc(I)c1 ; O=C1c2ccccc2C(=O)N1Cc1n[nH]c(=O)c2ccc(-c3cncc(Br)c3)cc12]

A mixture of 3-bromo-5-iodo-pyridine (3.00 g, 10.6 mmol, 1.00 eq.), intermediate AN (2.28 g, 5.28 mmol, 0.50 eq.), sodium bicarbonate (1.78 g, 21.1 mmol, 822 μL, 2.00 eq.), Pd(dppf)Cl₂ (773 mg, 1.06 mmol, 0.10 eq.) in dioxane (50 mL) and water (10 mL) was degassed with nitrogen 3 then stirred at 80° C. for 1 hour. The cooled reaction mixture was then concentrated under reduced, diluted with water (200 mL), filtered and the filter cake was triturated with dichloromethane:methyl alcohol (10%, 150 mL). The solid was filtered, dried and the solid was triturated a second time in methyl alcohol (100 mL), then filtered and dried to give 2-[[7-(5-bromo-3-pyridyl)-4-oxo-3H-phthalazin-1-yl]methyl]isoindoline-1,3-dione, Intermediate CB (775 mg, crude) as a gray solid. ¹H NMR (400 MHz, DMSO-d₆) δ=12.54 (s, 1H), 9.16 (d, J=1.2 Hz, 1H), 8.82 (d, J=1.6 Hz, 1H), 8.73 (s, 1H), 8.52 (s, 1H), 8.38-8.28 (m, 2H), 7.98-7.95 (m, 2H), 7.90 (m, 2H), 5.38 (s, 2H).

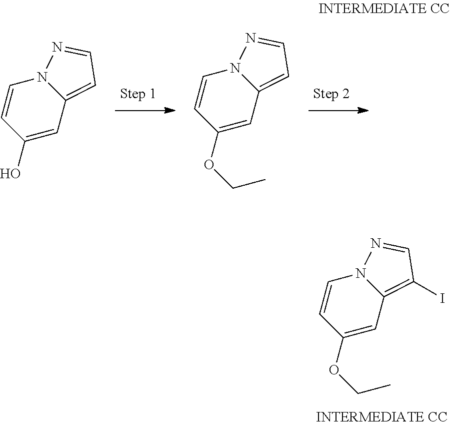

Step 1: To a solution of pyrazolo[1,5-a]pyridin-5-ol (250 mg, 1.86 mmol, 1.00 eq.) in DMF (2 mL) was added potassium carbonate (773 mg, 5.59 mmol, 3.00 eq.) and the mixture stirred at 30° C. for 0.5 hour. Iodoethane (872 mg, 5.59 mmol, 447 μL, 3.00 eq.) was then added and the resulting mixture stirred at 30° C. for 12 hours. After such time the reaction mixture was diluted with water (50 mL) extracted with ethyl acetate (20 mL×3) and the combined organic layers were washed with brine (50 mL), dried over anhydrous sodium sulfate, filtered and concentrated under reduced pressure. The residue was purified by prep-TLC (SiO₂, Petroleum ether/Ethyl acetate 20%) to give 5-ethoxypyrazolo[1,5-a]pyridine (272 mg, 1.68 mmol, 900% yield) as a white solid. LCMS [M+1]⁺=163.2; ¹H NMR (400 MHz, DMSO-d₆) δ=8.50 (d, J=7.6 Hz, 1H), 7.86 (d, J=2.0 Hz, 1H), 6.98 (d, J=2.8 Hz, 1H), 6.52 (dd, J=2.8, 7.6 Hz, 1H), 6.35 (d, J=2.0 Hz, 1H), 4.06 (q, J=6.8 Hz, 2H), 1.35 (t, J=6.8 Hz, 3H).

Step 2: To a solution of 5-ethoxypyrazolo[1,5-a]pyridine (260 mg, 1.60 mmol, 1.00 eq.) in acetonitrile (1.0 mL) was added NIS (397 mg, 1.76 mmol, 1.10 eq.). The mixture was stirred at 25° C. for 1 hour before the mixture was diluted with water (30 mL) and extracted with ethyl acetate (10 mL×3). The combined organic layers were washed with brine (20 mL), dried over anhydrous sodium sulfate, filtered and concentrated under reduced pressure. The residue was purified by prep-TLC (SiO₂, Petroleum ether/Ethyl acetate 20%) to give 5-ethoxy-3-iodo-pyrazolo[1,5-a]pyridine (369 mg, 1.28 mmol, 80% yield) as a pink solid. LCMS [M+1]=289.1; ¹H NMR (400 MHz, DMSO-d₆) δ=8.57 (d, J=7.6 Hz, 1H), 7.96 (s, 1H), 6.67 (d, J=2.4 Hz, 1H), 6.59 (dd, J=2.4, 7.6 Hz, 1H), 4.14 (q, J=6.8 Hz, 2H), 1.37 (t, J=6.8 Hz, 3H).

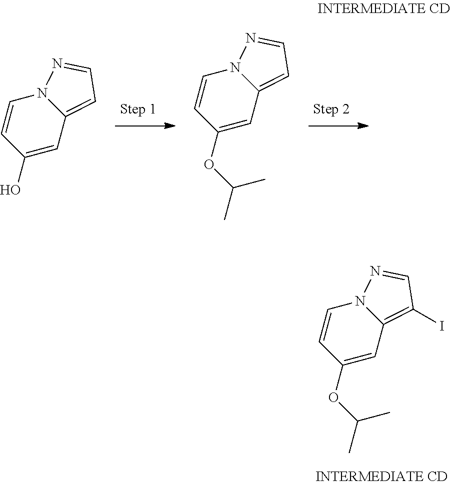

Intermediate CD, 3-iodo-5-isopropoxy-pyrazolo[1,5-a]pyridine was prepares as a yellow solid (299 mg, 0.99 mmol, 87% yield over 2 steps) using 2-iodopropane following the same procedure as used for the preparation of Intermediate CC. LCMS [M+1]⁺=303.0; ¹H NMR (400 MHz, DMSO-d₆) δ=8.56 (d, J=7.6 Hz, 1H), 7.96 (s, 1H), 6.67 (d, J=2.8 Hz, 1H), 6.57 (dd, J=2.8, 7.6 Hz, 1H), 4.77 (td, J=6.0, 12.0 Hz, 1H), 1.32 (s, 3H), 1.30 (s, 3H).

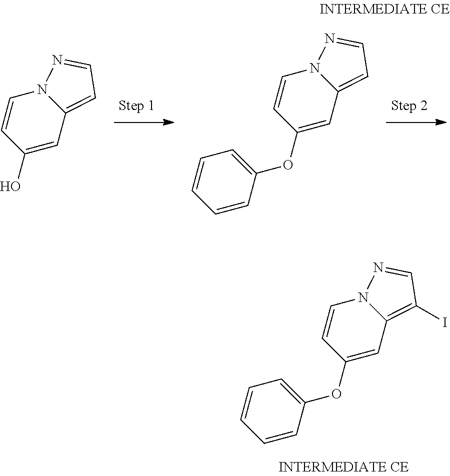 [SMILES: Oc1ccn2nccc2c1 ; c1ccc(Oc2ccn3nccc3c2)cc1 ; Ic1cnn2ccc(Oc3ccccc3)cc12]

Step 1: A mixture of pyrazolo[1,5-a]pyridin-5-ol (300 mg, 2.24 mmol, 1.00 eq.), phenylboronic acid (545 mg, 4.47 mmol, 2.00 eq.), 4 Å MS (30 mg), copper acetate (812 mg, 4.47 mmol, 2.00 eq.) and triethylamine (1.13 g, 11.2 mmol, 1.56 mL, 5.00 eq.) in dichloromethane (10 mL) was degassed with oxygen and stirred at 25° C. for 10 hours under an oxygen (15 psi) atmosphere. After such time the reaction mixture was filtered, concentrated and the formed residue was purified by column chromatography (SiO₂, petroleum ether/ethyl acetate 0-20%) to give 5-phenoxypyrazolo[1,5-a]pyridine (200 mg, 0.95 mmol, 43% yield) as a yellow oil. LCMS [M+1]⁺=211.2.

Step 2: To a solution of 5-phenoxypyrazolo[1,5-a]pyridine (180 mg, 0.86 mmol, 1.00 eq.) in acetonitrile (2 mL) was added NIS (212 mg, 0.94 mmol, 1.10 eq.). The mixture was stirred at 0° C. for 1 hour. The reaction mixture was then concentrated and the residue purified by column chromatography (SiO₂, petroleum ether/ethyl acetate 0-5%) to give 3-iodo-5-phenoxy-pyrazolo[1,5-a]pyridine (170 mg, 0.51 mmol, 59% yield) as a yellow oil. LCMS [M+1]⁺=336.9.

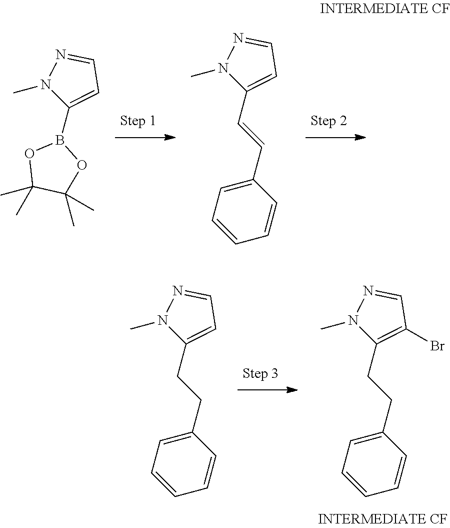

Step 1: A mixture of 1-methyl-5-(4,4,5,5-tetramethyl-1,3,2-dioxaborolan-2-yl)pyrazole (1.50 g, 7.21 mmol, 1.00 eq.), [(E)-2-bromovinyl]benzene (2.90 g, 15.8 mmol, 2.03 mL, 2.20 eq.), Pd(PPh₃)₂Cl₂ (506 mg, 721 μmol, 0.10 eq.), potassium carbonate (1.30 g, 9.41 mmol, 1.30 eq.) in ethyl alcohol (3.8 mL) and DMF (7.5 mL) was degassed with nitrogen and then stirred at 75° C. for 2 hours. After such time the mixture was cooled to ambient temperature, diluted with water (100 mL) and extracted with ethyl acetate (80 mL×3). The combined organic layers were washed with brine (150 mL), dried over anhydrous sodium sulfate, filtered and concentrated under reduced pressure. The residue was purified by column chromatography (SiO₂, Petroleum ether/ethyl acetate 0-20%) to give 1-methyl-5-[(E)-styryl]pyrazole (990 mg, 5.37 mmol, 74% yield) as a yellow solid. LCMS [M+1]⁺=185.2; ¹H NMR (400 MHz, DMSO-d₆) δ=7.65 (d, J=7.2 Hz, 2H), 7.42-7.36 (m, 3H), 7.32-7.25 (m, 2H), 7.12 (d, J=16.0 Hz, 1H), 6.63 (d, J=2.0 Hz, 1H), 3.91 (s, 3H).

Step 2: To a solution of 1-methyl-5-[(E)-styryl]pyrazole (400 mg, 2.17 mmol, 1.00 eq.) in ethyl alcohol (3 mL) was added Pd/C (10.0 mg, 10% Pd) under nitrogen. The suspension was degassed under vacuum and purged with hydrogen several times and the mixture was then stirred under hydrogen (15.0 psi) at 25° C. for 12 hours. After such time the reaction mixture was filtered and concentrated under reduced pressure to give 1-methyl-5-(2-phenylethyl)pyrazole (385 mg, 1.93 mmol, 89% yield) as a yellow oil and used into the next step without further purification. LCMS [M+1]⁺=187.2.

Step 3: To a solution of 1-methyl-5-(2-phenylethyl)pyrazole (385 mg, 1.93 mmol, 1.00 eq.) in acetonitrile (10 mL) was added N-bromosuccinimide (343 mg, 1.93 mmol, 1.00 eq.). The mixture was then stirred at 0° C. for 0.5 hour. After such time the reaction mixture was concentrated under reduced pressure and the residue purified by prep-TLC (SiO₂, Petroleum ether/ethyl acetate 20/6) to give 4-bromo-1-methyl-5-(2-phenylethyl)pyrazole (430 mg, 1.62 mmol, 84% yield) as a yellow oil. ¹H NMR (400 MHz, DMSO-d₆) δ=7.43 (s, 1H), 7.31-7.24 (m, 2H), 7.23-7.18 (m, 1H), 7.17-7.11 (m, 2H), 3.58 (s, 3H), 2.97-2.89 (t, J=7.2 Hz, 2H), 2.84-2.77 (t, J=7.2 Hz, 2H).

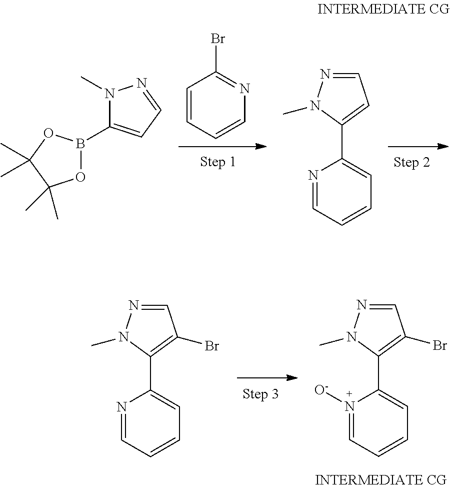

Step 1: A mixture of 1-methyl-5-(4,4,5,5-tetramethyl-1,3,2-dioxaborolan-2-yl)pyrazole (1.00 g, 4.81 mmol, 1.00 eq.), 2-bromopyridine (911 mg, 5.77 mmol, 0.55 mL, 1.20 eq.), cesium carbonate (3.13 g, 9.61 mmol, 2.00 eq.) and Pd(dppf)Cl₂ (352 mg, 0.48 mmol, 0.10 eq.) in dioxane (10 mL) and water (2 mL) was degassed and purged with nitrogen and then the mixture was stirred at 100° C. for 1 hour. After such time the cooled reaction mixture was concentrated under reduced pressure and purified by column chromatography (SiO₂, petroleum ether/ethyl acetate 0-20%) to give 2-(2-methylpyrazol-3-yl)pyridine (860 mg, crude) as a red oil which used into the next step directly without further purification. ¹H NMR (400 MHz, DMSO-d₆) δ=8.69-8.66 (m, 1H), 7.89 (dt, J=1.6, 7.6 Hz, 1H), 7.78 (td, J=1.2, 8.0 Hz, 1H), 7.48 (d, J=2.0 Hz, 1H), 7.37 (ddd, J=1.2, 4.8, 7.6 Hz, 1H), 6.78 (d, J=1.6 Hz, 1H), 4.14 (s, 3H).

Step 2: To a solution of 2-(2-methylpyrazol-3-yl)pyridine (760 mg, crude) in acetonitrile (10 mL) was added N-bromosuccinimide (850 mg, 4.77 mmol). The mixture was stirred at 0° C. for 0.5 hour. The reaction mixture was then concentrated under reduced pressure and the residue purified by prep-TLC (SiO₂, petroleum ether/ethyl acetate 20%) to give 2-(4-bromo-2-methyl-pyrazol-3-yl)pyridine (507 mg, 2.13 mmol, 44% yield) as an off-white solid. ¹H NMR (400 MHz, DMSO-d₆) δ=8.77 (td, J=0.8, 4.0 Hz, 1H), 8.01 (dt, J=2.0, 7.6 Hz, 1H), 7.76-

Step 3: To a solution of 2-(4-bromo-2-methyl-pyrazol-3-yl)pyridine (150 mg, 0.63 mmol, 1.00 eq.) in dichloroethane (3 mL) was added meta-chloroperbenzoic acid (435 mg, 2.14 mmol, 85% purity, 3.40 eq.). The mixture was stirred at 60° C. for 5 hours. The reaction mixture was then quenched by addition saturated sodium sulfite solution (20 mL) and extracted with dichloromethane (20 mL×3). The combined organic layers were washed with brine (30 mL), dried over anhydrous sodium sulfate, filtered and concentrated and the residue purified by prep-TLC (SiO₂, petroleum ether/ethyl acetate 50%) to give 2-(4-bromo-1-methyl-1H-pyrazol-5-yl)pyridine 1-oxide (185 mg, crude) as a yellow solid, which was used into the next step directly without further purification. LCMS [M+1]⁺=254.1; ¹H NMR (400 MHz, DMSO-d₆) δ=8.46 (d, J=6.4 Hz, 1H), 7.70 (s, 1H), 7.5 (dt, J=1.2, 7.6 Hz, 2H), 7.52-7.47 (m, 1H), 3.74 (s, 3H).

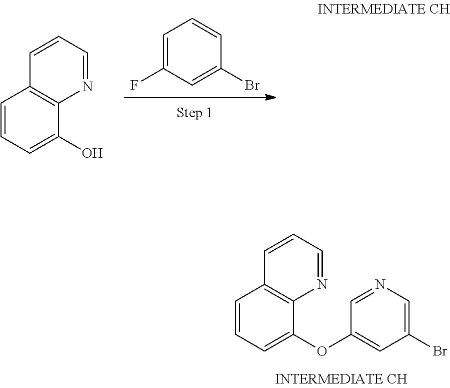 [SMILES: Oc1cccc2cccnc12 ; Fc1cccc(Br)c1 ; Brc1cncc(Oc2cccc3cccnc23)c1]

Step 1: A mixture of quinolin-8-ol (454 mg, 3.13 mmol, 0.54 mL, 1.10 eq.), 3-bromo-5-fluoro-pyridine (500 mg, 2.84 mmol, 1.00 eq.), potassium carbonate (785 mg, 5.68 mmol, 2.00 eq.) in DMF (6 mL) was degassed with nitrogen then stirred at 110° C. for 3 hours. After such time the mixture was extracted with ethyl acetate (5 mL×3) and the combined extracts were washed with brine (2 mL), dried over anhydrous sodium sulfate, filtered and concentrated to give 8-[(5-bromo-3-pyridyl)oxy]quinoline (0.30 g, 0.75 mmol, 26% yield) as a yellow oil. LCMS [M+1]⁺=301.0. ¹H NMR (400 MHz, CDCl₃) δ=8.95 (dd, J=2.0, 4.0 Hz, 1H), 8.43 (d, J=2.0 Hz, 1H), 8.39 (d, J=2.4 Hz, 1H), 8.25 (dd, J=2.0, 8.4 Hz, 1H), 7.73 (dd, J=1.6, 8.4 Hz, 1H), 7.55 (t, J=8.0 Hz, 1H), 7.50 (dd, J=4.0, 8.4 Hz, 1H), 7.47-7.43 (m, 2H).

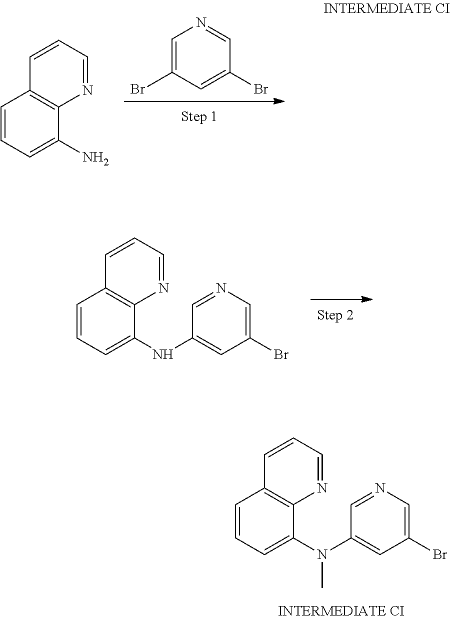 [SMILES: Nc1cccc2cccnc12 ; Brc1cncc(Br)c1 ; Brc1cncc(Nc2cccc3cccnc23)c1 ; CN(c1cncc(Br)c1)c1cccc2cccnc12]

Step 1: A mixture of 3,5-dibromopyridine (1.48 g, 6.25 mmol, 1.00 eq.), quinolin-8-amine (901 mg, 6.25 mmol, 1.00 eq.), sodium tert-butoxide (901 mg, 9.37 mmol, 1.50 eq.), Pd₂(dba)₃ (57.2 mg, 62.5 μmol, 0.01 eq.) and Xantphos (72.3 mg, 125 μmol, 0.02 eq.) in dioxane (10 mL) was degassed with nitrogen then stirred at 100° C. for 2 hours. After such time the mixture was diluted with water (20 mL) and extracted with ethyl acetate (20 mL×3). The combined organic phases were washed with brine (20 mL), dried over anhydrous sodium sulfate, filtered, concentrated and the residue purified by reversed-phase HPLC (0.1% formic acid (FA) condition) to give N-(5-bromo-3-pyridyl) quinolin-8-amine (160 mg, 486 μmol, 7% yield) as a yellow solid. LCMS [M+1]⁺=300.0; ¹H NMR (400 MHz, CDCl₃) δ=8.82 (dd, J=1.6, 4.0 Hz, 1H), 8.61 (d, J=2.4 Hz, 1H), 8.40 (br s, 1H), 8.31 (d, J=1.6 Hz, 1H), 8.17 (dd, J=1.6, 8.4 Hz, 1H), 7.92 (t, J=2.0 Hz, 1H), 7.51-7.46 (m, 3H), 7.37 (dd, J=1.6, 8.0 Hz, 1H).

Step 2: N-(5-bromo-3-pyridyl)quinolin-8-amine (130 mg, 394 μmol, 1.00 eq.) was dissolved in DMF (2 mL), then sodium hydride (32 mg, 790 μmol, 60.0% purity, 2.00 eq.) was added at 0° C. and the mixture was stirred at 0° C. for 10 minutes. After such time methyl iodide (224 mg, 1.58 mmol, 98 μL, 4.00 eq.) was added and the resulting mixture was stirred at 20° C. for 1 hour. The reaction mixture was then quenched with water (10 mL), extracted with ethyl acetate (20 mL×3) and the combined organic phases were washed with brine (20 mL), dried over anhydrous sodium sulfate, filtered and concentrated to give N-(5-bromo-3-pyridyl)-N-methyl-quinolin-8-amine (150 mg, 334 μmol, 85% yield) as a yellow oil. LCMS [M+1]⁺=314.1; ¹H NMR (400 MHz, CDCl₃) δ=8.90 (dd, J=1.6, 4.4 Hz, 1H), 8.24 (dd, J=1.6, 8.4 Hz, 1H), 8.01 (d, J=2.0 Hz, 1H), 7.92 (d, J=2.8 Hz, 1H), 7.82 (dd, J=1.6, 8.0 Hz, 1H), 7.65-7.62 (m, 1H), 7.61-7.57 (m, 1H), 7.46 (dd, J=4.4, 8.4 Hz, 1H), 7.10 (t, J=2.4 Hz, 1H), 3.49 (s, 3H).

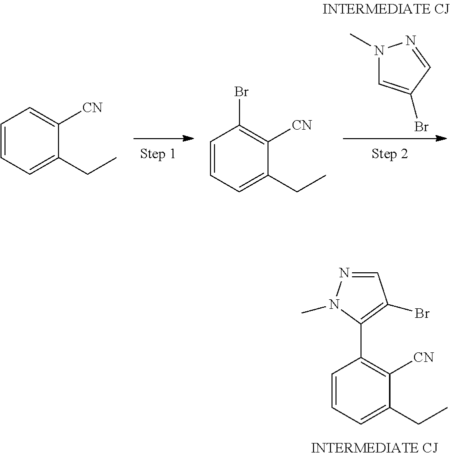

Step 1: A mixture of 2-ethylbenzonitrile (500 mg, 3.81 mmol, 0.51 mL, 1.00 eq.), p-toluenesulfonic acid (363 mg, 1.91 mmol, 0.50 eq.), N-Bromosuccinimide (746 mg, 4.19 mmol, 1.10 eq.) and palladium acetate (85.6 mg, 0.38 mol, 0.10 eq.) in 1,2-dichloroethane (10 mL) was degassed with nitrogen then stirred at 70° C. for 12 hours. After such time the reaction mixture was concentrated under reduced pressure and the residue was purified by column chromatography (SiO₂, petroleum ether/ethyl acetate 0-5%) to give 2-bromo-6-ethyl-benzonitrile (446 mg, 1.15 mmol, 30% yield) as a yellow oil. ¹H NMR (400 MHz, DMSO-d₆) δ=7.76 (dd, J=0.8, 7.6 Hz, 1H), 7.70 (d, J=8.0 Hz, 1H), 7.60-7.54 (m, 1H), 2.81 (m, 2H), 1.22 (m, 3H).

Step 2: A mixture of 2-bromo-6-ethyl-benzonitrile (446 mg, 1.15 mmol, 1.00 eq.), 4-bromo-1-methyl-pyrazole (203 mg, 1.26 mmol, 1.10 eq.), palladium acetate (2.57 mg, 0.12 mmol, 0.01 eq.), DavePhos (9.0 mg, 0.23 mmol, 0.02 eq.), 2-methylpropanoic acid (30.3 mg, 0.34 mmol, 31.9 μL, 0.30 eq.) and tetrabutylammonium acetate (691 mg, 2.29 mmol, 0.70 mL, 2.00 eq.) in N-methyl pyrrolidone (10 mL) was degassed with nitrogen then stirred at 100° C. for 12 hours. After such time the reaction mixture was diluted with water (100 mL), extracted with ethyl acetate (60 mL×3) and the combined organic layers were washed with brine (80 mL), dried over anhydrous sodium sulfate, filtered and concentrated under reduced pressure. The residue was purified by column chromatography (SiO₂, petroleum ether/ethyl acetate 10-20%) to give 2-(4-bromo-2-methyl-pyrazol-3-yl)-6-ethyl-benzonitrile (310 mg, 0.44 mmol, 39% yield) as a yellow solid. LCMS [M+1]⁺=290.1; ¹H NMR (400 Hz, DMSO-d₆) δ=7.83-7.78 (n, 1H), 7.73 (s, 1H), 7.68 (d, J=7.6 Hz, 1H), 7.50-7.47 (m, 1H), 3.71 (s, 3H), 2.90 (q, J=7.6 Hz, 2H), 1.28 (t, J=7.6 Hz, 3H).

The INTERMEDIATES D-1 to D-20 shown in Table I-V were prepared following the teachings of the General Reaction Schemes and the method to prepare INTERMEDIATE CJ.

TABLE I-V

| Intermediate | Structure | Spectral Data |
|---|---|---|
| D-1 | 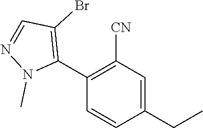 [SMILES: CCc1ccc(-c2c(Br)cnn2C)c(C#N)c1] | 2-(4-bromo-1-methyl-1H-pyrazol-5-y1)-5-ethylbenzonitrile LCMS [M + 1]⁺ = 289.9; ¹H NMR (400 MHz, CDCl₃) δ = 7.67 (d, J = 1.2 Hz, 1H), 7.60-7.55 (m, 2H), 7.38 (d, J = 8.0 Hz, 1H), 5.30 (s, 1H), 3.80 (s, 3H), 2.79 (q, J = 7.6 Hz, 2H), 1.33 (t, J = 7.6 Hz, 3H) |
| D-2I | 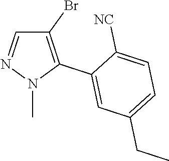 [SMILES: CCc1ccc(C#N)c(-c2c(Br)cnn2C)c1] | 2-(4-bromo-1-methyl-1H-pyrazol-5-y1)-4-ethylbenzonitrile LCMS [M + 1]⁺ = 290.0; ¹H NMR (400 MHz, CDCl₃) δ = 7.67 (d, J = 8.0 Hz, 1H), 7.49 (s, 1H), 7.36 (dd, J = 2.0, 8.0 Hz, 1H), 7.22 (d, J = 1.2 Hz, 1H), 3.72 (s, 3H), 2.72 (q, J = 7.6 Hz, 2H), 1.23 (t, J = 7.6 Hz, 3H) |
| D-3 | 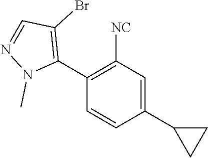 | 2-(4-bromo-1-methyl-1H-pyrazol-5-yl)-5-cyclopropylbenzonitrile LCMS [M + 1]⁺ = 304.1; ¹H NMR (400 MHz, CDCl₃) δ = 7.58 (s, 1H), 7.51 (d, J = 2.0 Hz, 1H), 7.43-7.39 (m, 1H), 7.36-7.31 (m, 1H), 3.79 (s, 3H), 2.01 (tt, J = 5.2, 8.4 Hz, 1H), 1.18-1.11 (m, 2H), 0.86-0.78 (m, 2H) |
| D-4 | 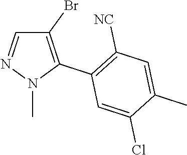 [SMILES: Cc1cc(C#N)c(-c2c(Br)cnn2C)cc1Cl] | 2-(4-bromo-1-methyl-1H-pyrazol-5-y1)-4-chloro-5- methylbenzonitrile LCMS [M + 1]⁺ = 312.0; ¹H NMR (400 MHz, CDCl₃) δ = 7.71 (s, 1H), 7.59 (s, 1H), 7.47 (s, 1H), 3.81 (s, 3H), 2.51 (s, 3H) |
| D-5 | 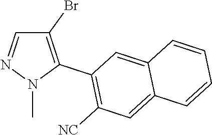 [SMILES: Cn1ncc(Br)c1-c1cc2ccccc2cc1C#N] | 3-(4-bromo-1-methyl-1H-pyrazol-5-y1)-2-naphthonitrile LCMS [M + 1]⁺ = 312.1; ¹H NMR (400 MHz, CDCl₃) δ = 8.43 (s, 1H), 8.04-7.96 (m, 2H), 7.95 (s, 1H), 7.79-7.70 (m, 2H), 7.64 (s, 1H), 3.85 (s, 3H) |
| D-6 | 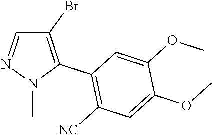 [SMILES: COc1cc(C#N)c(-c2c(Br)cnn2C)cc1OC] | 2-(4-bromo-1-methyl-1H-pyrazol-5-y1)-4,5-dimethoxybenzonitrile LCMS [M + 1]⁺ = 324.0; ¹H NMR (400 MHz, CDCl₃) δ = 7.59 (s, 1H), 7.23 (s, 1H), 6.87 (s, 1H), 3.99 (s, 3H), 3.97 (s, 3H), 3.83 (s, 3H) |
| D-7 | 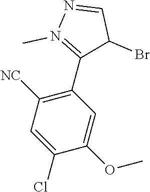 | 2-(4-bromo-1-methyl-1H-pyrazol-5-y1)-5-chloro-4- methoxybenzonitrile LCMS [M + 1]⁺ = 327,9; ¹H NMR (400 MHz, CDCl₃) δ = 7.83 (s, 1H), 7.61 (s, 1H), 6.96 (s, 1H), 4.01 (s, 3H), 3.84 (s, 3H) |
| D-8 | 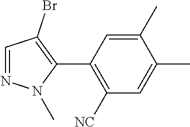 [SMILES: Cc1cc(C#N)c(-c2c(Br)cnn2C)cc1C] | 2-(4-bromo-1-methyl-1H-pyrazol-5-y1)-4,5-dimethylbenzonitrile LCMS [M + 1]⁺ = 292.0; ¹H NMR (400 MHz, CDCl₃) δ = 7.60 (s, 1H), 7.58 (s, 1H), 7.22 (s, 1H), 3.79 (s, 3H), 2.40 (s, 3H), 2.39 (S, 3H) |
| D-9 | 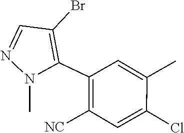 [SMILES: Cc1cc(-c2c(Br)cnn2C)c(C#N)cc1Cl] | 2-(4-bromo-1-methyl-1H-pyrazol-5-yl)-5-chloro-4- methylbenzonitrile LCMS [M + 1]⁺ = 312.0; ¹H NMR (400 MHz, CDCl₃) δ = 7.82 (s, 1H), 7.59 (s, 1H), 7.34 (s, 1H), 3.81 (s, 3H), 2.53 (s, 3H) |
| D-10 | 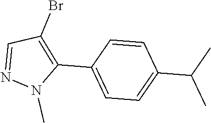 [SMILES: CC(C)c1ccc(-c2c(Br)cnn2C)cc1] | 4-bromo-5-(4-isopropylphenyl)-1-methyl-1H-pyrazole LCMS [M + 1]⁺ = 281.1; ¹H NMR (400 MHz, CDCl₃) δ = 7.54 (s, 1H), 7.39-7.32 (m, 4H), 3.83 (s, 3H), 1.31 (d, J = 6.8 Hz, 6H) |
| D-11 | 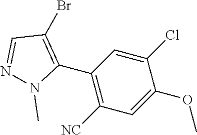 [SMILES: COc1cc(C#N)c(-c2c(Br)cnn2C)cc1Cl] | 2-(4-bromo-1-methyl-1H-pyrazol-5-y1)-4-chloro-5- methoxybenzonitrile LCMS [M + 1]⁺ = 328.0; ¹H NMR (400 MHz, MeOD) δ = 7.67 (s, 1H), 7.64 (s, 1H), 7.62 (s, 1H), 4.03 (s, 3H), 3.77 (s, 3H) |
| D-12 | 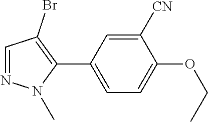 [SMILES: CCOc1ccc(-c2c(Br)cnn2C)cc1C#N] | 2-(4-bromo-1-methyl-1H-pyrazol-5-yl)-5-ethoxyterephthalonitrile LCMS [M + 1]⁺ = 306.1; ¹H NMR (400 MHz, CDCl₃) δ = 7.53 (d, J = 2.0 Hz, 1H), 7.49 (dd, J = 2.4, 8.8 Hz, 1H), 7.44 (s, 1H), 7.03 (d, J = 8.8 Hz, 1H), 4.16 (q, J = 7.2 Hz, 2H), 3.73 (s, 3H), 1.45 (t, J = 7.2 Hz, 3H) |
| D-13 | 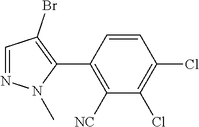 | 6-(4-bromo-1-methyl-1H-pyrazol-5-yl)-2,3-dichlorobenzonitrile LCMS [M + 1]⁺ = 392.3; ¹H NMR (400 MHz, CDCl₃) δ = 7.83 (d, J = 8.4 Hz, 1H), 7.61 (s, 1H), 7.34 (d, J = 8.4 Hz, 1H), 3.82 (s, 3H) |
| D-14 | 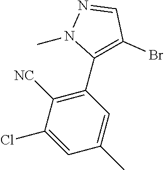 [SMILES: Cc1cc(Cl)c(C#N)c(-c2c(Br)cnn2C)c1] | 2-(4-bromo-1-methyl-1H-pyrazol-5-y1)-6-chloro-4- methylbenzonitrile LCMS [M + 1]⁺ = 311.9; ¹H NMR (400 MHz, CDCl₃) δ = 7.83 (s, 1H), 7.61 (s, 1H), 6.96 (s, 1H), 4.01 (s, 3H), 3.84 (s, 3H) |
| D-15 | 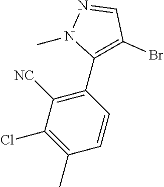 [SMILES: Cc1ccc(-c2c(Br)cnn2C)c(C#N)c1Cl] | 2-(4-bromo-1-methyl-1H-pyrazol-5-y1)-6-chloro-5- methylbenzonitrile LCMS [M + 1]⁺ = 312; ¹H NMR (400 MHz, CDCl₃) δ = 7.62 (d, J = 8.0 Hz, 1H), 7.59 (d, J = 2.0 Hz, 1H), 7.31-7.26 (m, 1H), 3.81 (d, J = 1.2 Hz, 3H), 2.54 (s, 3H) |
| D-16 | 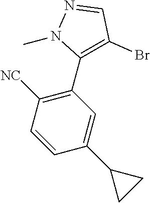 [SMILES: Cn1ncc(Br)c1-c1cc(C2CC2)ccc1C#N] | 2-(4-bromo-1-methyl-1H-pyrazol-5-y1)-4-cyclopropylbenzonitrile LCMS [M + 1]⁺ = 304.1; ¹H NMR (400 MHz, CDCl₃) δ = 7.71 (d, J = 8.4 Hz, 1H), 7.59 (s, 1H), 7.26-7.23 (dd, J = 1.6, 8.4, 1H), 7.11 (d, J = 1.6 Hz, 1H), 3.81 (s, 3H), 2.03-1.99 (m, 1H), 1.20-1.17 (m, 2H), 0.87-0.82 (m, 2H) |
| D-17 | 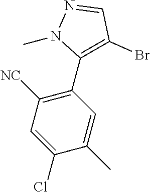 | 2-(4-bromo-1-methyl-1H-pyrazol-5-y1)-4,5-dichlorobenzonitrile LCMS [M + 1]⁺ = 329.9; ¹H NMR (400 MHz, CDCl₃) δ = 7.93 (s, 1H), 7.60 (m, 2H), 3.83 (s, 3H) |
| D-18 | 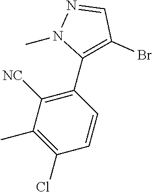 [SMILES: Cc1c(Cl)ccc(-c2c(Br)cnn2C)c1C#N] | 6-(4-bromo-1-methyl-1H-pyrazol-5-y1)-3-chloro-2- methylbenzonitrile LCMS [M + 1]⁺ = 312.0; ¹H NMR (400 MHz, CDCl₃) δ = 7.71 (d, J = 8.4Hz, 1H), 7.59 (s, 1H), 7.24 (d, J = 8.4 Hz, 1H), 3.79 (s, 3H), 2.71 (s, 3H) |
| D-19 | 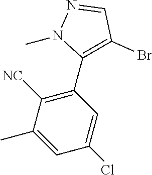 [SMILES: Cc1cc(Cl)cc(-c2c(Br)cnn2C)c1C#N] | 2-(4-bromo-1-methyl-LH-pyrazol-5-y1)-4-chloro-6- methylbenzo nitrile LCMS [M + 1]⁺ = 312.0; ¹H NMR (400 MHz, CDCl₃) δ = 7.59 (s, 1H), 7.48 (d, J = 1.2 Hz, 1H), 7.29 (d, J = 1.6 Hz, 1H), 3.81 (s, 3H) |
| D-20 | 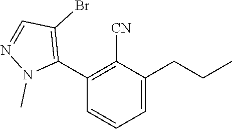 [SMILES: CCCc1cccc(-c2c(Br)cnn2C)c1C#N] | 2-(4-bromo-1-methyl-1H-pyrazol-5-y1)-6-propylbenzonitrile LCMS [M + 1]⁺ = 305.9; ¹H NMR (400 MHz, DMSO-d₆) δ = 7.84- 7.77 (m, 1H), 7.74 (s, 1H), 7.66 (d, J = 7.6 Hz, 1H), 7.49 (d, J = 7.6 Hz, 1H), 3.71 (s, 3H), 2.89-2.83 (m, 2H), 1.75-1.64 (m, 2H), 0.98- 0.92 (m, 3H) |

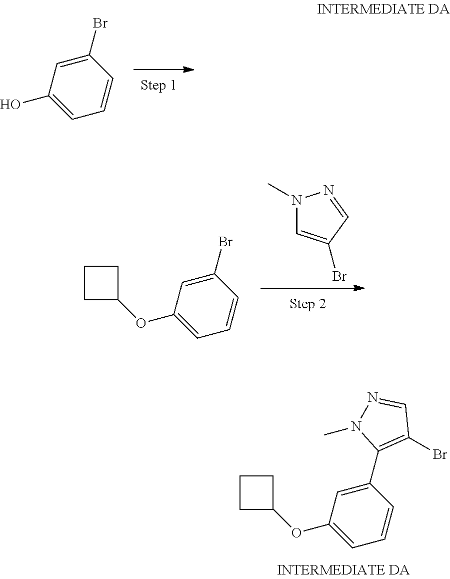

Step 1: A mixture of 3-bromophenol (1.00 g, 5.78 mmol, 1.00 eq.), bromocyclobutane (1.17 g, 8.65 mmol, 0.82 mL, 1.50 eq.) and potassium carbonate (3.20 g, 23.1 mmol, 4.00 eq.) in DMF (10 mL) and stirred at 120° C. for 6 hours. The reaction mixture was diluted with water (80 mL) and extracted with (petroleum ether/ethyl acetate 20%) (50 mL×3) and the combined extracts were washed with aqueous sodium hydroxide (1.00 M, 50 mL), brine (50 mL) and dried over sodium sulfate and concentrated to give 1-bromo-3-(cyclobutoxy)benzene (1.20 g, 5.27 mmol, 91% yield) as a colorless oil. ¹H NMR (400 MHz, CDCl₃) δ=7.04-6.93 (m, 2H), 6.86 (t, J=2.4 Hz, 1H), 6.64 (ddd, J=1.2, 2.4, 8.0 Hz, 1H), 4.60-4.44 (m, 1H), 2.34 (tddd, J=2.8, 6.8, 8.0, 9.6 Hz, 2H), 2.14-1.98 (m, 2H), 1.83-1.71 (m, 1H), 1.66-1.50 (m, 1H).

Step 2: 1-bromo-3-(cyclobutoxy)benzene (300 mg, 1.32 mmol, 1.00 eq.), 4-bromo-1-methyl-pyrazole (213 mg, 1.32 mmol, 1.00 eq.), palladium acetate (2.97 mg, 13.2 μmol, 0.01 eq.), tetrabutylammonium acetate (224 mg, 2.91 mmol, 2.20 eq.), 2-methylpropanoic acid (34.9 mg, 396 μmol, 36.8 μL, 0.30 eq.) and DavePhos (10.4 mg, 26.4 μmol, 0.02 eq.) in NMP (5 mL) was degassed with nitrogen and heated to 100° C. for 12 hours. The reaction mixture was then diluted with water (20 mL), extracted with ethyl acetate (30 mL×3) and the combined organic phases were washed with brine (30 mL), dried over anhydrous sodium sulfate, filtered and concentrated under reduced pressure. The residue was purified by column chromatography (SiO₂, petroleum ether/ethyl acetate 1-5%) to give 4-bromo-5-[3-(cyclobutoxy)phenyl]-1-methyl-pyrazole (40.0 mg) as a yellow oil. ¹H NMR (400 MHz, CDCl₃) δ=7.54 (s, 1H), 7.45 (s, 1H), 6.98-6.90 (m, 2H), 6.86-6.83 (m, 1H), 4.68 (t, J=7.2 Hz, 1H), 3.83 (s, 3H), 2.54-2.40 (m, 2H), 2.27-2.14 (m, 2H), 1.95-1.66 (m, 2H).

The INTERMEDIATES E-1 and E-2 shown in Table I-VI were prepared following the teachings of the General Reaction Schemes and the method to prepare Intermediate DA.

TABLE I-VI

| Intermediate | Structure | Spectral Data |
|---|---|---|
| E-1 | 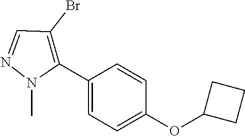 [SMILES: Cn1ncc(Br)c1-c1ccc(OC2CCC2)cc1] | 4-bromo-5-(4-cyclobutoxyphenyl)-1-methyl-1H-pyrazole LCMS [M + 1]⁺ = 307.1; ¹H NMR (400 MHz, CDCl₃) δ = 7.52 (s, 1H), 7.30 (d, J = 8.8 Hz, 2H), 6.93 (d, J = 8.8 Hz, 2H), 4.70 (quin, J = 7.2 Hz, 1H), 3.81 (s, 3H), 2.54-2.47 (m, 2H), 2.28-2.19 (m, 2H), 1.94-1.87 (m, 1H), 1.78-1.72 (m, 1H) |
| E-2 | 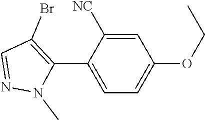 [SMILES: CCOc1ccc(-c2c(Br)cnn2C)c(C#N)c1] | 2-(4-bromo-1-methyl-1H-pyrazol-5-y1)-5-ethoxybenzonitrile LCMS [M + 1]⁺ = 307.9; ¹H NMR (400 MHz, CDCl₃) δ = 7.58 (s, 1H), 7.36 (d, J = 8.8 Hz, 1H), 7.30 (d, J = 2.8 Hz, 1H), 7.26-7.21 (dd, J = 2.8, 8.8 Hz, 1H), 4.16-4.10 (q, J = 7.2 Hz, 2H), 3.79 (s, 3H), 1.49 (t, J = 7.2 Hz, 3H) |

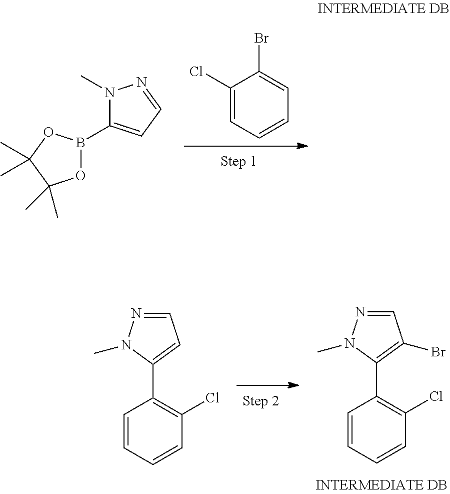

Step 1: A mixture of 1-methyl-5-(4,4,5,5-tetramethyl-1,3,2-dioxaborolan-2-yl)pyrazole (1.50 g, 7.21 mmol, 1.00 eq.), 1-bromo-2-chloro-benzene (1.38 g, 7.21 mmol, 0.84 mL, 1.00 eq.), sodium carbonate (2.29 g, 21.6 mmol, 3.00 eq.), Pd(dppf)Cl₂ (528 mg, 0.72 mmol, 0.10 eq.) in water (2.4 mL) and dioxane (12 mL) was degassed with nitrogen then stirred at 80° C. for 2 hours. After such time the mixture was concentrated under reduced pressure and purified by column chromatography (SiO₂, Petroleum ether/ethyl acetate 10-20%) to give 5-(2-chlorophenyl)-1-methyl-pyrazole (0.56 g, 2.88 mmol, 40% yield) as a yellow solid. LCMS [M+1]⁺=193.1; ¹H NMR (400 MHz, CDCl₃) δ=7.56 (d, J=2.0 Hz, 1H), 7.52 (dd, J=1.2, 7.6 Hz, 1H), 7.43-7.33 (m, 3H), 6.30 (d, J=2.0 Hz, 1H), 3.74 (s, 3H).

Step 2: A mixture of 5-(2-chlorophenyl)-1-methyl-pyrazole (200 mg, 1.04 mmol, 1.00 eq.), N-bromo-succinimide (203 mg, 1.14 mmol, 1.10 eq.) in acetonitrile (2 mL) was degassed with nitrogen then stirred at 0° C. for 2 hours. After such time the mixture was concentrated under reduced pressure and the residue was purified by prep-TLC (Petroleum ether/ethyl acetate 20%) to give 4-bromo-5-(2-chlorophenyl)-1-methyl-pyrazole (220 mg, 0.77 mmol, 74% yield) as a yellow solid. LCMS [M+1]⁺=273.1, ¹H NMR (400 MHz, CDCl₃) δ=7.59-7.55 (m, 2H), 7.50-7.40 (m, 2H), 7.36-7.33 (m, 1H), 3.74 (s, 3H).

The INTERMEDIATES F-1 to F-22 shown in Table I-VII were prepared following the teachings of the General Reaction Schemes and the method to prepare INTERMEDIATE DB.

TABLE I-VI

| Intermediate | Structure | Spectral Data |
|---|---|---|
| F-1 | 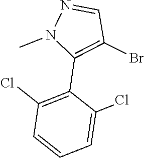 | 4-bromo-5-(2,6-dichloropheny1)-1-methyl-1H-pyrazole LCMS [M + 1]⁺ = 306.9; ¹H NMR (400 MHz, CDCl₃) δ = 7.59 (s, 1H), 7.53-7.51 (m, 1H), 7.50-7.47 (m, 1H), 7.46-7.35 (m, 1H), 3.70 (s, 3H) |
| F-2 | 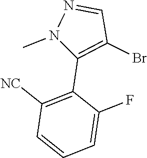 [SMILES: Cn1ncc(Br)c1-c1c(F)cccc1C#N] | 2-(4-bromo-1-methyl-1H-pyrazol-5-y1)-3-fluorobenzonitrile LCMS [M + 1]⁺ = 282.0; ¹H NMR (400 MHz, CDCl₃) δ = 7.72-7.64 (m, 2H), 7.63 (s, 1H), 7.55-7.49 (m, 1H), 3.80 (s, 3H) |
| F-3 | 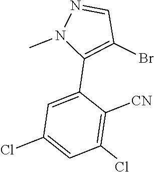 [SMILES: Cn1ncc(Br)c1-c1cc(Cl)cc(Cl)c1C#N] | 2-(4-bromo-1-methyl-1H-pyrazol-5-y1)-4,6-dichlorobenzonitrile LCMS [M + 1]⁺ = 331.9; ¹H NMR (400 MHz, CDCl₃) δ = 7.70 (d, J = 2.0 Hz, 1H), 7.61 (s, 1H), 7.39 (d, J = 2.0 Hz, 1H), 3.84 (s, 3H) |
| F-4 | 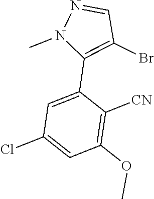 [SMILES: COc1cc(Cl)cc(-c2c(Br)cnn2C)c1C#N] | 2-(4-bromo-1-methyl-1H-pyrazol-5-y1)-4-chloro-6- methoxybenzonitrile LCMS [M + 1⁺] = 327.9; ¹H NMR (400 MHz, CDCl₃) δ = 7.57 (s, 1H), 7.11 (d, J = 2.0 Hz, 1H), 7.03 (d, J = 2.0 Hz, 1H), 4.02 (s, 3H), 3.82 (s, 3H) |
| F-5 | 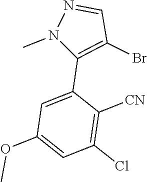 [SMILES: COc1cc(Cl)c(C#N)c(-c2c(Br)cnn2C)c1] | 2-(4-bromo-1-methyl-1H-pyrazol-5-y1)-4-methoxy-6- chlorobenzonitrile LCMS [M + 1]⁺ = 328.0; ¹H NMR (400 MHz, DMSO-d₆) δ = 7.75 (s, 1H), 7.56 (d, J = 2.4 Hz, 1H), 7.28 (d, J = 2.4 Hz, 1H), 3.93 (s, 3H), 3.75 (s, 3H) |
| F-6 | 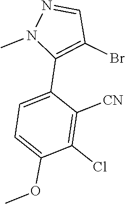 [SMILES: COc1ccc(-c2c(Br)cnn2C)c(C#N)c1Cl] | 2-(4-bromo-1-methyl-1H-pyrazol-5-y1)-5-methoxy-6- chlorobenzonitrile LCMS [M + 1]⁺ = 328.1; ¹H NMR (400 MHz, CDCl₃) δ = 7.58 (s, 1H), 7.34 (d, J = 8.4 Hz, 1H), 7.27 (d, J = 8.8 Hz, 1H), 4.03 (s, 3H), 3.80 (s, 3H) |
| F-7 | 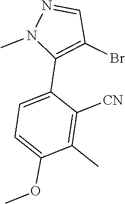 [SMILES: COc1ccc(-c2c(Br)cnn2C)c(C#N)c1C] | 2-(4-bromo-1-methyl-1H-pyrazol-5-y1)-5-methoxy-6- methylbenzonitrile LCMS [M + 1]⁺ = 308.0; ¹H NMR (400 MHz, CDCl₃) δ = 7.58 (s, 1H), 7.34 (d, J = 8.8 Hz, 1H), 7.27 (d, J = 8.8 Hz, 1H), 4.03 (s, 3H), 3.80 (s, 3H), 2.50 (s, 3H) |
| F-8 | 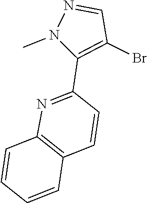 [SMILES: Cn1ncc(Br)c1-c1ccc2ccccc2n1] | 2-(4-bromo-1-methyl-1H-pyrazol-5-y1)quinoline LCMS [M + 1]⁺ = 290.0; ¹H NMR (400 MHz, CDCl₃) δ = 8.21 (d, J = 8.8 Hz, 1H), 8.07 (d, J = 8.4 Hz, 1H), 7.81 (d, J = 8.8 Hz, 2H), 7.70 (dt, J = 1.2, 7.6 Hz, 1H), 7.58-7.51 (m, 1H), 7.50 (s, 1H), 4.08 (s, 3H) |
| F-9 | 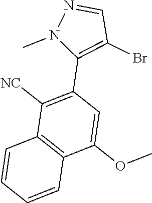 [SMILES: COc1cc(-c2c(Br)cnn2C)c(C#N)c2ccccc12] | 2-(4-bromo-1-methyl-1H-pyrazol-5-yl)-4-methoxy-1-naphthonitrile LCMS [M + 1]⁺ = 344.0; ¹H NMR (400 MHz, CDCl₃) δ = 8.40 (d, J = 8.0 Hz, 1H), 8.29 (d, J = 8.0 Hz, 1H), 7.79 (ddd, J = 1.2, 7,2, 8.4 Hz, 1H), 7.70 (ddd, J = 1,2, 7.2, 8.4 Hz, 1H), 7.64 (s, 1H), 6.80 (s, 1H), 4.12 (s, 3H), 3.88 (s, 3H) |
| F-10 | 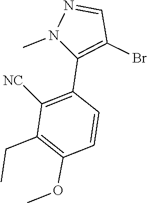 [SMILES: CCc1c(OC)ccc(-c2c(Br)cnn2C)c1C#N] | 6-(4-bromo-1-methyl-1H-pyrazol-5-yl)-2-ethyl-3-methoxybenzonitrile LCMS [M + 1]⁺ = 322.1; ¹H NMR (400 MHz, CDCl₃) δ = 7.56 (s, 1H), 7.26-7.22 (m, 1H), 7.18-7.11 (m, 1H), 3.94 (s, 3H), 3.77 (s, 3H), 2.96 (q, J = 7.6 Hz, 2H), 1.28-1.24 (m, 3H) |
| F-11 | 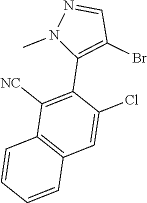 [SMILES: Cn1ncc(Br)c1-c1c(Cl)cc2ccccc2c1C#N] | 2-(4-bromo-1-methyl-1H-pyrazol-5-y1)-3-chloro-1-naphthonitrile LCMS [M + 1]⁺ = 346.0, 348.0; ¹H NMR (400 MHz, DMSO-d₆) δ = 8.78 (s, 1H), 8.23 (t, J = 9.2, 2H), 7.95-7.90 (m, 2H), 7.85 (s, 1H), 3.74 (s, 1H) |
| F-12 | 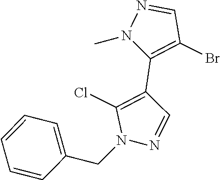 [SMILES: Cn1ncc(Br)c1-c1cnn(Cc2ccccc2)c1Cl] | l′-benzyl-4-bromo-5′-chloro-2-methyl-1′H,2H-3,4′-bipyrazole LCMS [M + 1]⁺ = 353.0; ¹H NMR (400 MHz, CDCl₃) δ = 7.67 (s, 1H), 7.54 (s, 1H), 7.41-7.33 (m, 3H), 7.31-7.27 (m, 2H), 5.43 (s, 2H), 3.79 (s, 3H) |
| F-13 | 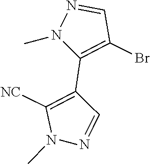 | 4-bromo-1′,2-dimethyl-1′H,2H-[3,4′-bipyrazole]-5′-carbonitrile LCMS [M + 1]⁺ = 265.9; ¹H NMR (400 MHz, CDCl₃) δ = 7.70 (s, 1H), 7.56 (s, 1H), 4.16 (s, 3H), 3.87 (s, 3H) |
| F-14 | 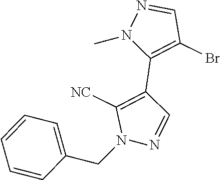 [SMILES: Cn1ncc(Br)c1-c1cnn(Cc2ccccc2)c1C#N] | 1′-benzyl-4-bromo-2-methyl-1′H,2H-[3,4′-bipyrazole]-5′-carbonitrile LCMS [M + 1]⁺ = 342.0 |
| F-15 | 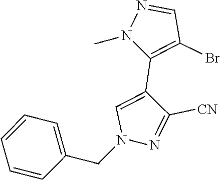 [SMILES: Cn1ncc(Br)c1-c1cn(Cc2ccccc2)nc1C#N] | l′-benzyl-4-bromo-2-methyl-1′H,2H-[3,4′-bipyrazole]-3′-carbonitrile LCMS [M + 1]⁺ = 342.0; ¹H NMR (400 MHz, CDCl₃-d) δ = 7.61 (s, 1H), 7.54 (s, 1H), 7.47-7.39 (m, 1H), 7.36-7.30 (m, 2H), 5.43 (s, 2H), 3.87 (s, 3H) |
| F-16 | 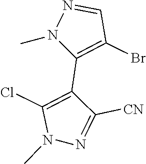 [SMILES: Cn1nc(C#N)c(-c2c(Br)cnn2C)c1Cl] | 4-bromo-5′-chloro-1′,2-dimethyl-1′H,2H-[3,4′-bipyrazole]-3′- carbonitrile LCMS [M + 1]⁺ = 302.0; ¹H NMR (400 MHz, CDCl₃) δ = 7.59 (s, 1H), 4.04 (s, 3H), 3.84 (s, 3H) |
| F-17 | 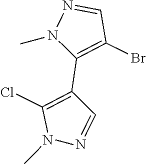 | 4-bromo-5′-chloro-1′,2-dimethyl-1′H,2H-3,4′-bipyrazole LCMS [M + 1]⁺ =277.0; ¹H NMR (400 MHz, CDCl₃) δ = 7.60 (s, 1H), 7.53 (s, 1H), 3.94 (s, 3H), 3.79 (s, 3H) |
| F-18 | 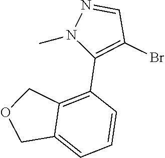 [SMILES: Cn1ncc(Br)c1-c1cccc2c1COC2] | 4-bromo-5-(1,3-dihydroisobenzofuran-4-y1)-1-methyl-1H-pyrazole LCMS [M + 1]⁺ = 280.9; ¹H NMR (400 MHz, CDCl₃) δ = 7.54 (s, 1H), 7.45-7.35 (m, 2H), 7.18 (d, J = 7.2 Hz, 1H), 5.26-5.11 (m, 3H), 4.88- 4.83 (m, 1H), 3.72 (s, 3H) |
| F-19 | 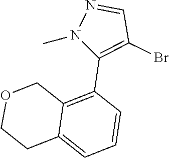 [SMILES: Cn1ncc(Br)c1-c1cccc2c1COCC2] | 4-bromo-5-(isochroman-8-y1)-1-methyl-1H-pyrazole LCMS [M + 1]⁺ = 293.0; ¹H NMR (400 MHz, CDCl₃) δ = 7.54 (s, 1H), 7.34-7.26 (m, 2H), 7.04 (d. J = 6.8 Hz, 1H), 4.65-4.57 (m, 1H), 4.34- 4.28 (m, 1H), 4.07-3.92 (m, 2H), 3.65 (s, 3H), 3.05-2.88 (m, 2H) |
| F-20 | 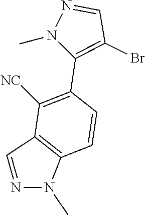 [SMILES: Cn1ncc(Br)c1-c1ccc2c(cnn2C)c1C#N] | 5-(4-bromo-1-methyl-1H-pyrazol-5-yl)-1-methyl-1H-indazole-4- carbonitrile LCMS [M + 1]⁺ = 318.0 |
| F-21 | 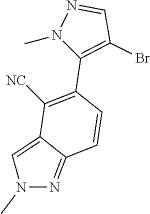 [SMILES: Cn1cc2c(C#N)c(-c3c(Br)cnn3C)ccc2n1] | 5~(4-bromo-1-methyl-H-pyrazol-5-y1)-2-methyl-2H-indazole-4- carbonitrile LCMS [M + 1]⁺ = 318.0 |
| F-22 | 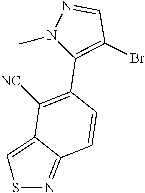 [SMILES: Cn1ncc(Br)c1-c1ccc2nscc2c1C#N] | 5-(4-bromo-1-methyl-1H-pyrazol-5-yl)benzo[c]isothiazole-4- carbonitrile LCMS [M + 1]⁺ = 318.9; ¹H NMR (400 MHz, CDCl₃,) δ = 9.62 (d, J = 0.8 Hz, 1H), 8.22 (dd, J = 1.2, 9.2 Hz, 1H), 7.65 (s, 1H), 7.50 (d, J = 9.2 Hz, 1H), 3.88 (s, 3H) |

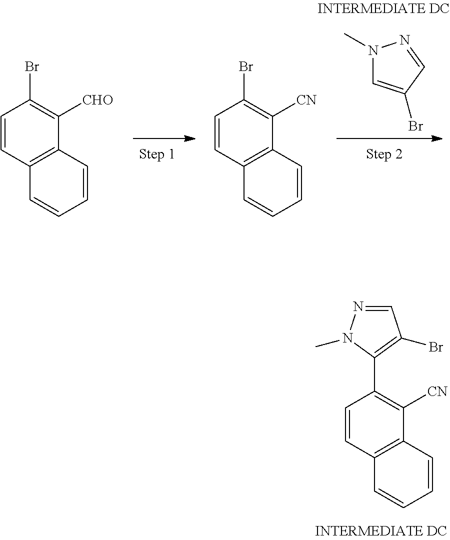

Step 1: To a solution of 2-bromonaphthalene-1-carbaldehyde (220 mg, 0.94 mmol, 1.00 eq.) in water (5 mL) was added amino hydrogen sulfate (212 mg, 1.87 mmol, 2.00 eq.). The mixture was stirred at 50° C. for 12 hours. The suspension was then filtered and the filter cake dried under reduced pressure to give (1E)-2-bromonaphthalene-1-carbaldehyde oxime (220 mg, 0.88 mmol, 94% yield) as a white solid which used without further purification. ¹H NMR (400 MHz, DMSO-d₆) δ=11.79 (s, 1H), 8.58 (s, 1H), 8.55 (dd, J=1.6, 8.0 Hz, 1H), 8.03-7.98 (m, 1H), 7.92 (d, J=8.8 Hz, 1H), 7.77 (d, J=8.8 Hz, 1H), 7.67-7.57 (m, 2H). To a solution of (IE)-2-bromonaphthalene-1-carbaldehyde oxime (220 mg, crude) in THF (5 mL) was added triethylamine (890 mg, 8.80 mmol, 1.22 mL) and trifluoroacetic anhydride (924 mg, 4.40 mmol, 0.61 mL) and the mixture stirred at 20° C. for 1 hour. After such time the reaction mixture was concentrated under reduced pressure and the residue was purified by prep-TLC (SiO₂, petroleum ether/ethyl acetate 10%) to give 2-bromonaphthalene-1-carbonitrile (190 mg, 0.82 mmol, 93% yield) as a white solid. GCMS [M+1]⁺=230.9; ¹H NMR (400 MHz, DMSO-d₆) δ=8.26 (d, J=8.8 Hz, 1H), 8.16 (d, J=8.0 Hz, 1H), 8.09 (d, J=8.0 Hz, 1H), 7.93 (d, J=8.8 Hz, 1H), 7.85 (dt, J=1.2, 8.4 Hz, 1H), 7.79-7.71 (m, 1H).

Step 2: A mixture of 2-bromonaphthalene-1-carbonitrile (190 mg, 0.82 mmol, 1.00 eq.), 4-bromo-1-methyl-pyrazole (132 mg, 0.82 mmol, 1.00 eq.), tetrabutylammonium acetate (494 mg, 1.64 mmol, 0.50 mL, 2.00 eq.), DavePhos (6.4 mg, 16 μmol, 0.02 eq.), 2-methylpropanoic acid (22 mg, 246 μmol, 23 μL, 0.30 eq.) and palladium acetate (1.8 mg, 8.2 μmol, 0.01 eq.) in N-methyl pyrrolidone (NMP) (6 mL) was degassed with nitrogen then the mixture was stirred at 100° C. for 12 hours. After such time the reaction mixture was diluted with water (30 mL) and extracted with ethyl acetate (50 mL×3). The combined organic layers were washed with brine (100 mL×2), dried over anhydrous sodium sulfate, filtered and concentrated under reduced pressure. The residue was purified by reversed-phase HPLC (0.1% formic acid condition) to give 2-(4-bromo-2-methyl-pyrazol-3-yl)naphthalene-1-carbonitrile (190 mg, 0.61 mmol, 74% yield) as a yellow oil. LCMS [M+1]⁺=314.1; ¹H NMR (400 MHz, DMSO-d₆) δ=8.49 (d, J=8.4 Hz, 1H), 8.24 (t, J=8.4 Hz, 2H), 7.92 (dt, J=1.2, 8.4 Hz, 1H), 7.87-7.82 (m, 1H), 7.81 (s, 1H), 7.76 (d, J=8.4 Hz, 1H), 3.79 (s, 3H).

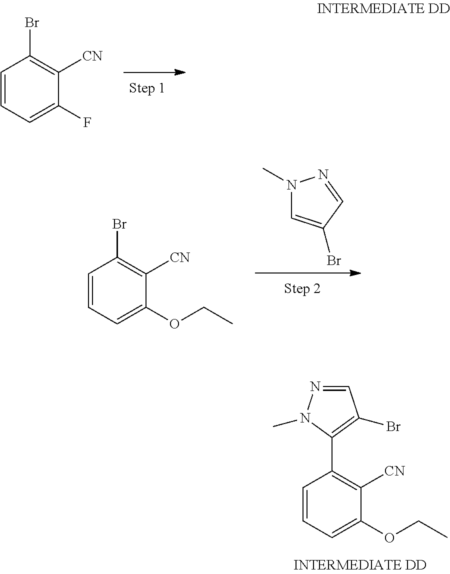

Step 1: To a solution of ethyl alcohol (207 mg, 4.50 mmol, 0.26 mL, 3.00 eq.) in THF (3 mL) was added sodium hydride (180 mg, 4.50 mmol, 60.0% purity, 3.00 eq.), followed by a solution of 2-bromo-6-fluoro-benzonitrile (300 mg, 1.50 mmol, 1.00 eq.) in THF (1 mL) in a dropwise fashion. After the addition was complete the mixture was stirred at 25° C. for 3 hours. After such time the reaction was quenched with water (0.2 mL) and concentrated in vacuum and the residue was purified by column chromatography (SiO₂, petroleum ether/ethyl acetate 10%) to give 2-bromo-6-ethoxy-benzonitrile (200 mg, 0.89 mmol, 59% yield) as a white solid. ¹H NMR (400 MHz, CDCl₃) δ=7.38-7.32 (t, J=8.4 Hz, 1H), 7.20 (d, J=8.0 Hz, 1H), 6.90 (d, J=8.4 Hz, 1H), 4.15 (q, J=7.2 Hz, 2H), 1.47 (t, J=7.2 Hz, 3H).

Step 2: A mixture of 2-bromo-6-ethoxy-benzonitrile (200 mg, 0.89 mmol, 1.00 eq.), 4-bromo-1-methyl-pyrazole (185 mg, 1.15 mmol, 1.30 eq.), palladium acetate (2.0 mg, 8.9 μmol, 0.01 eq.), DavePhos (7.0 mg, 17.7 μmol, 0.02 eq.), 2-methylpropanoic acid (23.4 mg, 265 μmol, 25 μL, 0.30 eq.) and tetrabutylammonium acetate (533 mg, 1.77 mmol, 2.00 eq.) was degassed with nitrogen then the mixture was stirred at 100° C. for 15 hours. After such time the mixture was diluted with ethyl acetate (20 mL), washed with water (20 mL×3) and the combined organic phases were dried over anhydrous sodium sulfate, filtered and concentrated in vacuum. The residue was then purified by prep-TLC (SiO₂, petroleum ether/ethyl acetate 20%) to give 2-(4-bromo-1-methyl-1H-pyrazol-5-yl)-6-ethoxybenzonitrile (60.0 mg, 0.20 mmol, 22% yield) as a white solid. LCMS [M+1]⁺=306.1; ¹H NMR (400 MHz, CDCl₃) δ=7.56 (dd, J=7.6, 8.4 Hz, 1H), 7.49 (s, 1H), 7.02 (d, J=8.4 Hz, 1H), 6.91 (d, J=7.6 Hz, 1H), 4.15 (q, J=7.2 Hz, 2H), 3.73 (s, 3H), 1.46 (t, J=7.2 Hz, 3H).

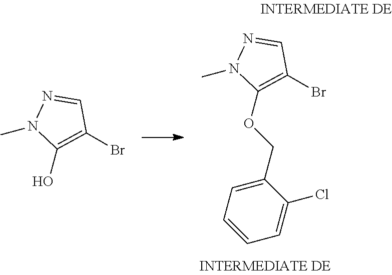

A mixture of 4-bromo-2-methyl-pyrazol-3-ol (300 mg, 1.69 mmol, 1.00 eq.), 1-(bromomethyl)-2-chloro-benzene (348 mg, 1.69 mmol, 0.22 mL, 1.00 eq.), and potassium carbonate (469 mg, 3.39 mmol, 2.00 eq.) in DMF (8 mL) was was stirred at 18° C. for 2 hours. After such time the reaction mixture was diluted with water (20 mL) and extracted with ethyl acetate (20 mL×3). The combined organic extracts were washed with brine (20 mL), dried over anhydrous sodium sulfate, filtered, concentrated under reduced pressure and the residue purified by reversed-phase HPLC (0.1% FA condition) to give 4-bromo-5-[(2-chlorophenyl)methoxy]-1-methyl-pyrazole (220 mg, 0.72 mmol, 42% yield) as a yellow solid. LCMS [M+1]⁺=303.0; ¹H NMR (400 MHz, CDCl₃) δ=7.48-7.43 (m, 2H), 7.37-7.27 (m, 3H), 5.40 (s, 2H), 3.55 (s, 3H).

The INTERMEDIATES to G-1 to G-4 shown in Table I-VIII were prepared following the teachings of the General Reaction Schemes and the method to prepare INTERMEDIATE DE.

TABLE I-VIII

| Intermediate | Structure | Spectral Data |
|---|---|---|
| G-1 | 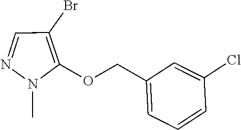 [SMILES: Cn1ncc(Br)c1OCc1cccc(Cl)c1] | 4-bromo-5-((3-chlorobenzyl)oxy)-1-methyl-1H-pyrazole LCMS [M + 1]⁺ = 303.0; ¹H NMR (400 MHz, CDCl₃) δ = 7.43 (d, J = 1.6 Hz, 1H), 7.37-7.35 (m, 1H), 7.34-7.30 (m, 2H), 7.28 (t, J = 1.6 Hz, 1H), 5.26 (s, 2H), 3.54 (s, 3H) |
| G-2 | 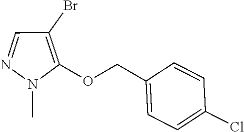 [SMILES: Cn1ncc(Br)c1OCc1ccc(Cl)cc1] | 4-bromo-5-((4-chlorobenzyl)oxy)-1-methyl-1H-pyrazole LCMS [M + 1]⁺ = 303.0; ¹H NMR (400 MHz, CDCl₃) δ = 7.43- 7.35 (m, 2H), 7.35-7.29 (m, 3H), 5.25 (s, 2H), 3.49 (s, 3H) |
| G-3 | 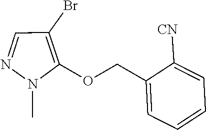 | 4-bromo-5-((2-cyanobenzyl)oxy)-1-methyl-1H-pyrazole LCMS [M + 1]⁺ = 294.1; ¹H NMR (400 MHz, CDCl₃) δ = 7.75 (d, J = 7.6 Hz, 1H), 7.69-7.59 (m, 2H), 7.55-7.49 (m, 1H), 7.32 (s, 1H), 5.46 (s, 2H), 3.64 (s, 3H) |
| G-4 | 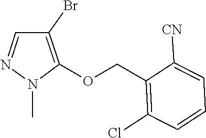 | 2-(((4-bromo-1-methyl-1H-pyrazol-5-y1)oxy)methyl)-3- chlorobenzonitrile LCMS [M + 1]⁺ = 327.9; ¹H NMR (400 MHz, CDCl₃) δ = 7.63- 7.58 (m, 1H), 7.58-7.54 (m, 2H), 7.34-7.30 (m, 1H), 5.46 (s, 2H), 3.68 (s, 3H) |

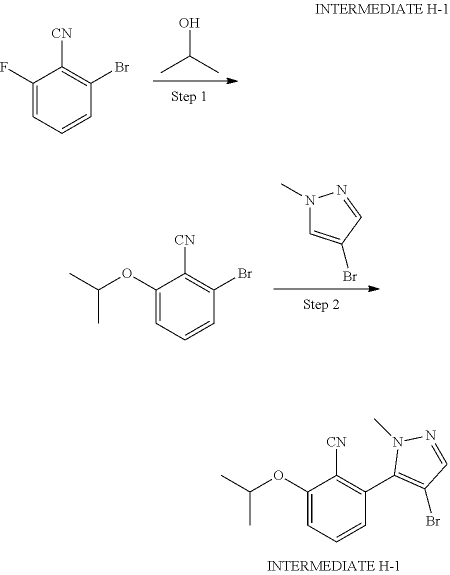

Step 1: A mixture of 2-bromo-6-fluoro-benzonitrile (600 mg, 3.00 mmol, 1.00 eq.), propan-2-ol (225 mg, 3.75 mmol, 0.29 mL, 1.25 eq.), cesium carbonate (1.47 g, 4.50 mmol, 1.50 eq.) in DMF (6 mL) was stirred at 75° C. for 1 hour. After such time the reaction mixture was diluted with water (30 mL) and extracted with ethyl acetate (50 mL×3). The combined organic layers were washed with brine (100 mL×2), dried over anhydrous sodium sulfate, filtered and concentrated under reduced pressure. The residue was purified by reversed-phase HPLC (0.1% formic acid condition) to give 2-bromo-6-isopropoxy-benzonitrile (540 mg, 2.25 mmol, 75% yield) as a white solid. LCMS [M+1]⁺=241.9; ¹H NMR (400 MHz, DMSO-d6) δ=7.56 (t, J=8.4 Hz, 1H), 7.37 (d, J=7.6 Hz, 1H), 7.31 (d, J=8.4 Hz, 1H), 4.90-4.75 (m, 1H), 1.32 (d, J=6.0 Hz, 6H).

Step 2: A mixture of 2-bromo-6-isopropoxy-benzonitrile (500 mg, 2.08 mmol, 1.00 eq.), 4-bromo-1-methyl-pyrazole (335 mg, 2.08 mmol, 1.00 eq.), diacetoxypalladium (4.7 mg, 0.021 mmol, 0.01 eq.), DavePhos (16 mg, 0.042 mmol, 0.02 eq.), tetrabutylammonium; acetate (1.26 g, 4.16 mmol, 2.00 eq.) and 2-methylpropanoic acid (55 mg, 0.63 mmol, 0.06 mL, 0.30 eq.) in 1-methyl-2-pyrrolidinone (7 mL) was degassed with nitrogen then stirred at 100° C. for 12 hours. After such time the reaction mixture was diluted with water (30 mL) and extracted with ethyl acetate (40 mL×3). The combined organic layers were washed with brine (100 mL×2), dried over anhydrous sodium sulfate, filtered and concentrated under reduced pressure. The residue was purified by reversed-phase HPLC (0.1% formic acid condition) to give 2-(4-bromo-2-methyl-pyrazol-3-yl)-6-isopropoxy-benzonitrile (160 mg, 0.50 mmol, 24 yield) as a white solid. LCMS [M+1]⁺=319.9; ¹H NMR (400 MHz, DMSO-d₆) δ=7.81-7.76 (m, 1H), 7.72 (s, 1H), 7.46 (d, J=8.8 Hz, 1H), 7.14 (d, J=7.2 Hz, 1H), 4.88 (td, J=6.0, 12.0 Hz, 1H), 3.71 (s, 3H), 1.36 (d, J=6.0 Hz, 6H).

The INTERMEDIATES to H-2 to H-8 shown in Table I-IX were prepared following the teachings of the General Reaction Schemes and the method to prepare INTERMEDIATE H-1.

TABLE I-IX

| Intermediate | Structure | Spectral Data |
|---|---|---|
| H-2 | 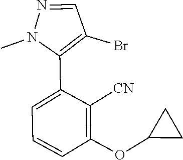 [SMILES: Cn1ncc(Br)c1-c1cccc(OC2CC2)c1C#N] | 2-(4-bromo-1-methyl-1H-pyrazol-5-y1)-6-cyclopropoxybenzonitrile LCMS [M + 1]⁺ = 318.0; ¹H NMR (400 MHz, CDCl₃) δ = 7.71-7.64 (m, 1H), 7.58 (s, 1H), 7.49 (d, J = 8.8 Hz, 1H), 7.05-7.01 (m, 1H), 3.92 (td, J = 2.8, 5,6 Hz, 1H), 0.96-0.91 (m, 4H) |
| H-3 | 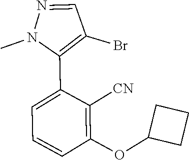 [SMILES: Cn1ncc(Br)c1-c1cccc(OC2CCC2)c1C#N] | 2-(4-bromo-1-methyl-1H-pyrazol-5-y1)-6-cyclobutoxybenzonitrile LCMS [M + 1]⁺ = 334.0; ¹H NMR (400 MHz, CDCl₃) δ = 7.65-7.59 (m, 1H), 7.59 (s, 1H), 7.00-6.93 (m, 2H), 4.80 (q, J = 7.2 Hz, 1H), 3.82- 3.79 (s, 3H), 2.61-2.48 (m, 2H), 2.39-2.27 (m, 2H), 2.03-1.91 (m, 1H), 1.84-1.70 (m, 1H) |
| H-4 | 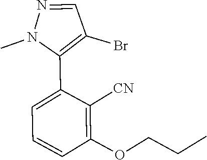 [SMILES: CCCOc1cccc(-c2c(Br)cnn2C)c1C#N] | 2-(4-bromo-1-methyl-1H-pyrazol-5-y1)-6-propoxybenzonitrile LCMS [M + 1]⁺ = 320.0; ¹H NMR (400 MHz, DMSO-d₆) δ = 7.80 (dd, J = 8.0, 8.4 Hz, 1H), 7.73 (s, 1H), 7.43 (d, J = 8.4 Hz, 1H), 7.17 (d, J = 7.6 Hz, 1H), 4.18 (dt, J = 2.0, 6.4 Hz, 2H), 1.81 (q, J = 7.6 Hz, 2H), 1.02 (t, J = 7.6 Hz, 3H) |
| H-5 | 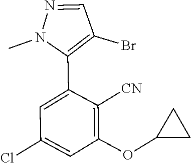 [SMILES: Cn1ncc(Br)c1-c1cc(Cl)cc(OC2CC2)c1C#N] | 2-(4-bromo-1-methyl-1/7-pyrazol-5-y1)-4-chloro-6- cyclopropoxybenzonitrile LCMS [M + 1]⁺ = 353.9; ¹H NMR (400 MHz, CDCl₃) δ = 7.57 (s, 1H), 7.48 (d, J = 2.0 Hz, 1H), 7.04 (d. J = 2.0 Hz, 1H), 3.95-3.90 (m, 1H), 3.81 (s, 3H), 0.99-0.94 (m, 4H) |
| H-6 | 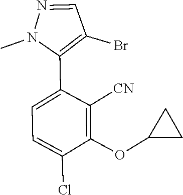 [SMILES: Cn1ncc(Br)c1-c1ccc(Cl)c(OC2CC2)c1C#N] | 6-(4-bromo-1-methyl-1H-pyrazol-5-yl)-3-chloro-2- cyclopropoxybenzonitrile LCMS [M + 1]⁺ = 354.0; ¹H NMR (400 MHz, CDCl₃) δ = 7.73 (d, J = 8.4 Hz, 1H), 7.60 (s, 1H), 7.13 (d, J = 8.4 Hz, 1H), 4.61 (m, 1H), 3.82 (s, 3H), 1.11-1.04 (m, 2H), 0.80-0.72 (m, 2H) |
| H-7 | 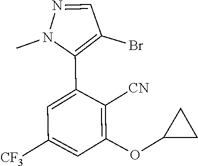 [SMILES: Cn1ncc(Br)c1-c1cc(C(F)(F)F)cc(OC2CC2)c1C#N] | 2-(4-bromo-1-methyl-1H-pyrazol-5-y1)-6-cyclopropoxy-4- (trifluoromethyl)benzonitrile ¹H NMR (500 MHz, CDCl₃) δ = 7.69 (d, J = 1.2 Hz, 1 H), 7.59 (s, 1 H), 7.29 (d, J = 1.2 Hz, 1 H), 4.01-3.97 (m, 1 H), 3.81 (s, 3 H), 1.01- 0.97 (m, 4 H) |
| H-8 | 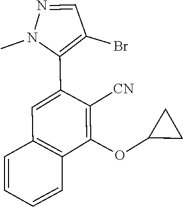 [SMILES: Cn1ncc(Br)c1-c1cc2ccccc2c(OC2CC2)c1C#N] | 3-(4-bromo-1-methyl-1H-pyrazol-5-y1)-1-cyclopropoxy-2- naphthonitrile ¹H NMR (400 MHz, CDCl₃) δ = 8.26 (d, J = 8.4 Hz, 1H), 7.90 (d, J = 8.4 Hz, 1H), 7.76-7.70 (m, 1H), 7.70-7.65 (m, 1H), 7.63 (s, 1H), 7.57 (s, 1H), 4.74 (tt, J = 2.8, 6.0 Hz, 1H), 3.86 (s, 3H), 1.13-1.07 (m, 2H), 0.95-0.87 (m, 2H) |

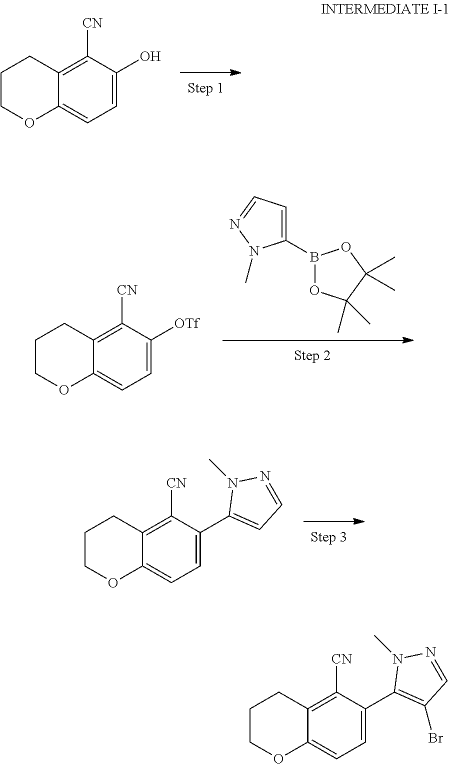 [SMILES: N#Cc1c(O)ccc2c1CCCO2 ; N#Cc1c(OS(=O)(=O)C(F)(F)F)ccc2c1CCCO2 ; Cn1nccc1B1OC(C)(C)C(C)(C)O1 ; Cn1nccc1-c1ccc2c(c1C#N)CCCO2 ; Cn1ncc(Br)c1-c1ccc2c(c1C#N)CCCO2]

Step 1: To a solution of 6-hydroxychromane-5-carbonitrile (150 mg, 0.86 mmol, 1.00 eq.) and triethylamine (2.57 mmol, 0.36 mL, 3.00 eq.) in dichloromethane (2 mL) was added a solution of trifluoromethanesulfonic anhydride (0.86 mmol, 0.141 mL, 1.00 eq.) in dichloromethane (1 mL) dropwise at 0° C., The mixture was then stirred at 0° C. for 0.5 hour. After such time the mixture was diluted with ethyl acetate (50 mL), washed with brine (50 mL×3) and dried over anhydrous sodium sulfate, filtered and concentrated. The residue was purified by prep-TLC (SiO₂, petroleum ether/ethyl acetate 100%) to give (5-cyanochroman-6-yl) trifluoromethanesulfonate (80 mg, 0.26 mmol, 30% yield) as a colorless liquid. ¹H NMR (400 MHz, CDCl₃) δ=7.21-7.15 (m, 1H), 7.09-7.04 (m, 1H), 4.31-4.20 (m, 2H), 3.00 (t, J=6.4 Hz, 2H), 2.18-2.02 (m, 2H).

Step 2: A mixture of (5-cyanochroman-6-yl) trifluoromethanesulfonate (70 mg, 0.23 mmol, 1.00 eq.), 1-methyl-5-(4,4,5,5-tetramethyl-1,3,2-dioxaborolan-2-yl)pyrazole (71. mg, 0.34 mmol, 1.50 eq.), Pd(dtbpf)Cl₂ (15 mg, 0.23 mmol, 0.10 eq.), sodium bicarbonate (38 mg, 0.46 mmol, 2.00 eq.) in DMF (2 mL) was degassed with nitrogen. The mixture was then stirred at 80° C. for 1 hour, cooled to 25° C., diluted with ethyl acetate (30 mL) and washed with brine (30 mL×3), dried over anhydrous sodium sulfate, filtered and concentrated under reduced pressure. The residue was purified by prep-TLC (SiO₂, petroleum ether/ethyl acetate 30%) to give 6-(2-methylpyrazol-3-yl)chromane-5-carbonitrile (40 mg, 0.17 mmol, 73% yield) as a white solid. LCMS [M+1]⁺=240.0; ¹H NMR (400 MHz, CDCl₃)=7.57 (d, J=1.6 Hz, 1H), 7.19-7.14 (m, 1H), 7.12-7.06 (m, 1H), 6.40 (d, J=2.0 Hz, 1H), 4.30-4.24 (m, 2H), 3.82 (s, 3H), 3.03 (t, J=6.4 Hz, 2H), 2.17-2.09 (m, 2H).

Step 3: To a solution of 6-(2-methylpyrazol-3-yl)chromane-5-carbonitrile (30 mg, 0.125 mmol, 1.00 eq.) in acetonitrile (1.5 mL) was added NBS (34 mg, 0.19 mmol, 1.50 eq.). The mixture was stirred at 25° C. for 1 hour then concentrated. The residue was purified by prep-TLC (SiO₂, petroleum ether/ethyl acetate 30%) to give 6-(4-bromo-2-methyl-pyrazol-3-yl)chromane-5-carbonitrile (25 mg, 0.79 mmol, 63% yield) as a yellow solid. LCMS [M+1]⁺=320.1; ¹H NMR (400 MHz, CDCl₃) δ=7.57 (s, 1H), 7.18-7.11 (m, 2H), 4.29 (dd, J=4.4, 6.0 Hz, 2H), 3.79 (s, 3H), 3.05 (dt, J=2.0, 6.4 Hz, 2H), 2.15 (dq, J=4.4, 6.4 Hz, 2H).

The INTERMEDIATES to I-2 to 1-4 shown in Table I-X were prepared following the teachings of the General Reaction Schemes and the method to prepare INTERMEDIATE I-1

TABLE I-X

| Intermediate | Structure | Spectral Data |
|---|---|---|
| I-2 | 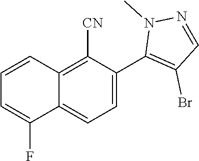 [SMILES: Cn1ncc(Br)c1-c1ccc2c(F)cccc2c1C#N] | 2-(4-bromo-1-methyl-1H-pyrazol-5-y1)-5-fluoro-1-naphthonitrile LCMS [M + 1] = 329.9; ¹H NMR (400 MHz, CDCl₃) δ = 8.49 (d, J = 8.8 Hz, 1H), 8.16 (d, J = 8.4 Hz, 1H), 7.75 (dt, J = 5.2, 8.0 Hz, 1H), 7.66 (s, 1H), 7.59 (d, J = 8.8 Hz, 1H), 7.41 (dd, J = 7.2, 9.2 Hz, 1H), 3.86 (s, 3H) |
| I-3 | 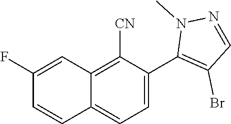 [SMILES: Cn1ncc(Br)c1-c1ccc2ccc(F)cc2c1C#N] | 2-(4-bromo-1-methyl-1H-pyrazol-5-y1)-7-fluoro-1-naphthonitrile LCMS [M + 1] = 329.9; ¹H NMR (400 MHz, CDCl₃) δ = 8.21 (d, J = 8.4 Hz, 1H), 8.04 (dd, J = 5.4, 9.1 Hz, 1H), 8.00 (dd, J = 2.4, 9.5 Hz, 1H), 7.65 (s, 1H), 7.56-7.47 (m, 2H), 3.86 (s, 3H) |
| I-4 | 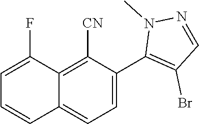 [SMILES: Cn1ncc(Br)c1-c1ccc2cccc(F)c2c1C#N] | 2-(4-bromo-1-methyl-1H-pyrazol-5-y1)-8-fluoro-1-naphthonitrile ¹H NMR (400 MHz, CDCl₃) δ = 8.22 (dd, J = 1.6, 8.4 Hz, 1H), 7.82 (d. J = 8.4 Hz, 1H), 7.71-7.66 (m, 1H), 7.65 (s, 1H), 7.55 (d, J = 8.4 Hz, 1H), 7.49-7.42 (m, 1H), 3.87 (s, 3H) |

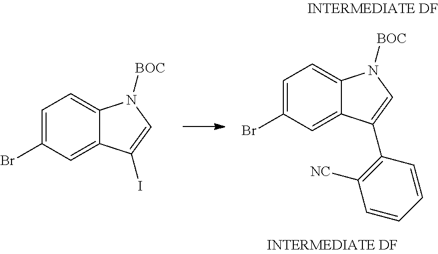 [SMILES: CC(C)(C)OC(=O)n1cc(I)c2cc(Br)ccc21 ; CC(C)(C)OC(=O)n1cc(-c2ccccc2C#N)c2cc(Br)ccc21]

A mixture of tert-butyl 5-bromo-3-iodo-pyrrolo[2,3-b]pyridine-1-carboxylate (120 mg, 0.28 mmol, 1 eq), (2-cyanophenyl)boronic acid (83 mg, 0.57 mmol, 2 eq), Pd(dppf)Cl₂ (21 mg, 0.03 mmol, 0.1 eq), NaHCO₃ (71 mg, 0.85 mmol) in DMF (2 mL) was degassed with nitrogen then stirred at 80° C. for 3 hr. After such time mixture was diluted with ethyl acetate (20 mL) and washed by water (20 mL×3). The organic phase was concentrated and the residue purified by by prep-TLC (SiO₂, petroleum ether/ethyl acetate 15%) to give intermediate E-1, tert-butyl 5-bromo-3-(2-cyanophenyl)pyrrolo[2,3-b]pyridine-1-carboxylate (50 mg, 0.13 mmol, 44% yield) as a white solid. LCMS [M−55]=342.1; ¹H NMR (400 MHz, CDCl₃) δ=8.62 (d, J=2.0 Hz, 1H), 8.06 (d, J=2.4 Hz, 1H), 8.03 (s, 1H), 7.84 (dd, J=1.2, 8.0 Hz, 1H), 7.76-7.70 (m, 1H), 7.63 (d, J=7.2 Hz, 1H), 7.52 (dt, J=1.2, 7.6 Hz, 1H), 1.70 (s, 9H).

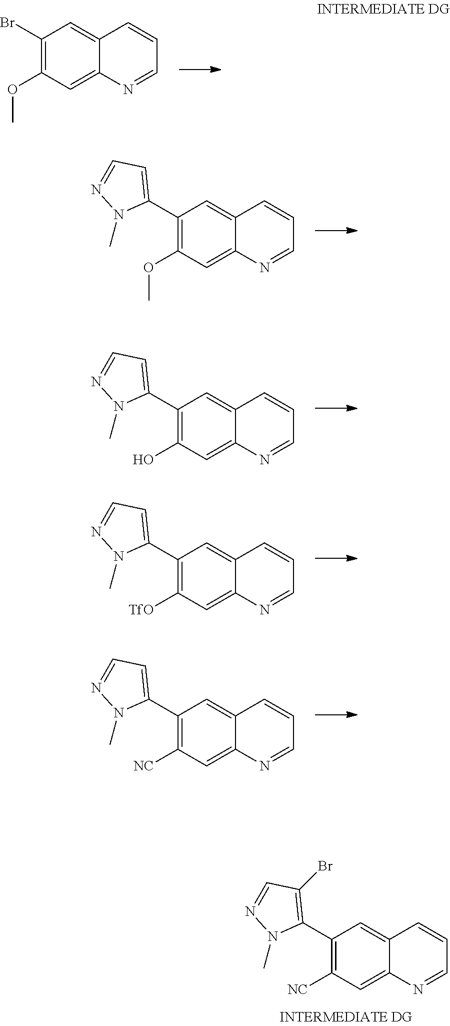 [SMILES: COc1cc2ncccc2cc1Br ; COc1cc2ncccc2cc1-c1ccnn1C ; Cn1nccc1-c1cc2cccnc2cc1O ; Cn1nccc1-c1cc2cccnc2cc1OS(=O)(=O)C(F)(F)F ; Cn1nccc1-c1cc2cccnc2cc1C#N ; Cn1ncc(Br)c1-c1cc2cccnc2cc1C#N]

Step 1: A mixture of 6-bromo-7-methoxy-quinoline (100 mg, 0.420 mmol, 1.00 eq.), 1-methyl-5-(4,4,5,5-tetramethyl-1,3,2-dioxaborolan-2-yl)pyrazole (105 mg, 0.504 mmol, 1.20 eq.), Pd(dtbpf)Cl₂ (27 mg, 0.042 mmol, 0.10 eq.) and sodium carbonate (89 mg, 0.840 mmol, 2.00 eq.) in dioxane (1.0 mL) and water (0.2 mL) was degassed with nitrogen. The mixture was then stirred at 80° C. for 2 hours, concentrated under reduced pressure and the residue purified by prep-TLC (SiO₂, ethyl acetate) to give 7-methoxy-6-(2-methylpyrazol-3-yl)quinoline (80 mg, 0.334 mmol, 80% yield) as a yellow solid. LCMS [M+1]⁺=240.2; ¹H NMR (400 MHz, CDCl₃) δ=8.82 (dd, J=1.6, 4.4 Hz, 1H), 8.04 (d, J=8.0 Hz, 1H), 7.65 (s, 1H), 7.51 (d, J=1.6 Hz, 1H), 7.48 (s, 1H), 7.27 (dd, =4.4, 8.0 Hz, 1H), 6.28 (d, J=1.6 Hz, 1H), 3.91 (s, 3H), 3.70 (s, 3H).

Step 2: A mixture of 7-methoxy-6-(2-methylpyrazol-3-yl)quinoline (500 mg, 2.09 mmol, 1.00 eq.) and pyridine hydrochloride (2.41 g, 20.9 mmol, 10.0 eq.) was stirred at 160° C. for 0.5 hour. After such time the residue was purified by reverse prep-HPLC (0.1% formic acid) to give 6-(2-methylpyrazol-3-yl) quinolin-7-ol (260 mg, 1.07 mmol, 51% yield, 92% purity) as a yellow solid. LCMS [M+1]⁺=226.1.

Step 3: To a solution of 6-(2-methylpyrazol-3-yl)quinolin-7-ol (260 mg, 1.15 mmol, 1.00 eq.) and triethylamine (0.32 mL, 2.31 mmol, 2.00 eq.) in dichloromethane (5 mL) was added trifluoromethanesulfonic anhydride (0.29 mL, 1.73 mmol, 1.50 eq.) in a dropwise fashion at 0° C. The mixture was stirred at 20° C. for 1 hour, quenched with water (12 mL) and extracted with dichloromethane (15 mL×3). The combined organic extracts were washed with brine (12 mL), dried over anhydrous sodium sulfate, filtered and concentrated and the residue purified by column chromatography (SiO₂, petroleum ether/ethyl acetate 10-100%) to give [6-(2-methylpyrazol-3-yl)-7-quinolyl] trifluoromethanesulfonate (0.97 g, 0.706 mmol, 61%) as a yellow oil. LCMS [M+1]⁺=358.1.

Step 4: A mixture of [6-(2-methylpyrazol-3-yl)-7-quinolyl] trifluoromethanesulfonate (970 mg, 0.668 mmol, 1.00 eq.), zinc cyanide (157 mg, 1.34 mmol, 2.00 eq.), Pd₂(dba)₃ (61 mg, 0.67 mmol, 0.1 eq.), DPPF (74 mg, 0.134 mmol, 0.20 eq.) and zinc powder (4.3 mg, 0.67 mmol, 0.10 eq.) in DMF (10 mL) was degassed with nitrogen then stirred at 100° C. for 2 hours. After such time the reaction mixture was diluted with water (20 mL) and extracted with ethyl acetate (20 mL×3). The combined organic phases were washed with brine (20 mL), dried over anhydrous sodium sulfate, filtered, concentrated and the residue purified by HPLC (0.1% formic acid condition) to give 6-(2-methylpyrazol-3-yl)quinoline-7-carbonitrile (100 mg, 0.249 mmol, 37% yield) as a brown solid. LCMS [M+1]⁺=235.2.

Step 5: To a solution of 6-(2-methylpyrazol-3-yl)quinoline-7-carbonitrile (90 mg, 0.384 mmol, 1.00 eq.) in acetonitrile (5 mL) was added N-bromosuccinimide (103 mg, 0.576 mmol, 1.50 eq.). The mixture was stirred at 20° C. for 0.5 hours then concentrated under reduced pressure and the residue was purified by prep-TLC (dichloromethane/methyl alcohol 10%) to give 6-(4-bromo-2-methyl-pyrazol-3-yl)quinoline-7-carbonitrile (50 mg, 0.160 mmol, 41% yield) as a yellow solid. LCMS [M+1]⁺=315.1; ¹H NMR (400 MHz, CDCl₃) δ=9.14 (dd, J=1.6, 4.4 Hz, 1H), 8.68 (s, 1H), 8.31 (d, J=8.4 Hz, 1H), 7.96 (s, 1H), 7.69-7.65 (m, 1H), 7.65 (s, 1H), 3.86 (s, 3H).

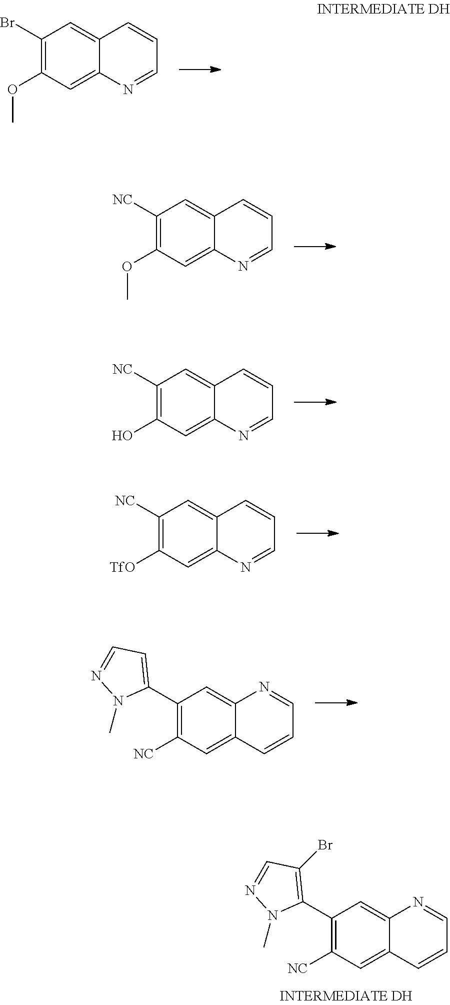 [SMILES: COc1cc2ncccc2cc1Br ; COc1cc2ncccc2cc1C#N ; N#Cc1cc2cccnc2cc1O ; N#Cc1cc2cccnc2cc1OS(=O)(=O)C(F)(F)F ; Cn1nccc1-c1cc2ncccc2cc1C#N ; Cn1ncc(Br)c1-c1cc2ncccc2cc1C#N]

Step 1: A mixture of 6-bromo-7-methoxy-quinoline (100 mg, 0.420 mmol, 1.00 eq.), zinc cyanide (98 mg, 0.840 mmol, 2.00 eq.), Pd₂(dba)₃ (38 mg, 0.042 mmol, 0.10 eq.), DPPF (47 mg, 0.084 mmol, 0.20 eq.) and zinc powder (2.8 mg, 0.042 mmol, 0.10 eq.) in DMF (2 mL) was degassed and purged with nitrogen. The mixture was then stirred at 100° C. for 2 hours then diluted with water (2 mL) and extracted with ethyl acetate (2 mL×3). The combined organic extracts were washed with brine (2 mL), dried over anhydrous sodium sulfate, filtered and concentrated. The residue was purified by HPLC (0.1% formic acid) to give 7-methoxyquinoline-6-carbonitrile (56 mg, 0.304 mmol, 72% yield) as a white solid. LCMS [M+1]⁺=185.2; ¹H NMR (400 MHz, CDCl₃) δ=8.97 (dd, J=1.6, 4.0 Hz, 1H), 8.17 (s, 1H), 8.15 (dd, J=1.2, 8.4 Hz, 1H), 7.53 (s, 1H), 7.40 (dd, J=4.0, 8.4 Hz, 1H), 4.09 (s, 3H).

Step 2: To a solution of 7-methoxyquinoline-6-carbonitrile (1.40 g, 7.60 mmol, 1.00 eq) in toluene (20 mL) was added aluminum trichloride (3.04 g, 22.8 mmol, 1.25 mL, 3.00 eq). The mixture was stirred at 100° C. for 1 hour and then the reaction mixture was diluted with water (3 mL) and pH adjusted to 4-5 with sodium hydroxide (2N, 0.1 mL). The formed solid was filtered and dried under reduced pressure to give 7-hydroxyquinoline-6-carbonitrile (1.20 g, crude) as a black solid which used into next step directly without further purification. LCMS [M+1]⁺=171.1.

Step 3: To a solution of 7-hydroxyquinoline-6-carbonitrile (500 mg, 2.94 mmol, 1 eq.) and triethylamine (0.82 mL, 5.88 mmol, 2.00 eq.) in dichloromethane (10 mL) was added trifluoromethanesulfonic anhydride (0.73 mL, 4.41 mmol, 1.50 eq.) in a dropwise fashion at 0° C. The mixture was stirred at 20° C. for 1 hour and after such time the reaction mixture was quenched with water (20 mL) and extracted with dichloromethane (50 mL×3). The combined organic extracts were washed with brine (20 mL), dried over anhydrous sodium sulfate, filtered and concentrated. The residue was purified by column chromatography (SiO₂, petroleum ether/ethyl acetate 10-50%) to give (6-cyano-7-quinolyl) trifluoromethanesulfonate (250 mg, 0.570 mmol, 19% yield) as a yellow oil. LCMS [M+1]⁺=303.0; ¹H NMR (400 MHz, CDCl₃) δ=9.15 (dd, J=1.6, 4.0 Hz, 1H), 8.37 (s, 1H), 8.32-8.30 (d, J=8.4 Hz 1H), 8.23 (s, 1H), 7.65 (dd, J=4.0, 8.4 Hz, 1H).

Step 4: A mixture of (6-cyano-7-quinolyl) trifluoromethanesulfonate (237 mg, 0.541 mmol, 1.00 eq.), 1-methyl-5-(4,4,5,5-tetramethyl-1,3,2-dioxaborolan-2-yl)pyrazole (135 mg, 0.649 mmol, 1.20 eq.), sodium bicarbonate (91 mg, 1.08 mmol, 2.00 eq.) and Pd(dtbpf)Cl₂ (35 mg, 0.054 mmol, 0.10 eq.) in dioxane (10 mL) and water (2 mL) was degassed with nitrogen and stirred at 80° C. for 1 hour. After such time the mixture was concentrated and the residue was purified by column chromatography (SiO₂, petroleum ether/ethyl acetate 10-1000%) to give 7-(2-methylpyrazol-3-yl)quinoline-6-carbonitrile (120 mg, 0.498 mmol, 92% yield) as a yellow solid. LCMS [M+1]⁺=235.2.

Step 5: A mixture of 7-(2-methylpyrazol-3-yl)quinoline-6-carbonitrile (120 mg, 0.512 mmol, 1.00 eq.) and N-bromosuccinimide (164 mg, 0.922 mmol, 1.80 eq.) in acetonitrile (4 mL) was degassed with nitrogen and stirred at 20° C. for 2 hours. After such time the mixture was concentrated and the residue was purified by prep-TLC (SiO₂, dichloromethane/methyl alcohol 10%) to give 7-(4-bromo-2-methyl-pyrazol-3-yl)quinoline-6-carbonitrile (121 mg, 0.385 mmol, 75% yield) as a yellow solid. LCMS [M+1]⁺=314.9; ¹H NMR (400 MHz, CDCl₃) δ=9.15 (dd, J=2.0, 4.4 Hz, 1H), 8.43 (s, 1H), 8.33 (dd, J=0.8, 8.4 Hz, 1H), 8.23 (s, 1H), 7.68-7.63 (m, 2H), 3.87 (s, 3H).

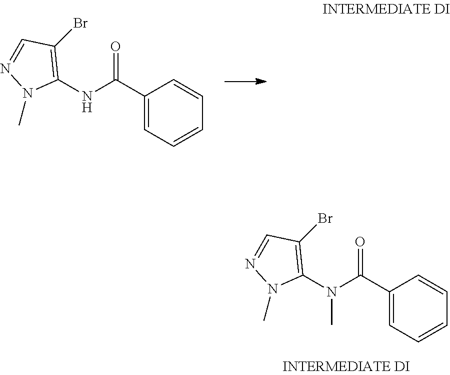 [SMILES: Cn1ncc(Br)c1NC(=O)c1ccccc1 ; CN(C(=O)c1ccccc1)c1c(Br)cnn1C]

To a solution of N-(4-bromo-2-methyl-pyrazol-3-yl)benzamide (500 mg, 1.78 mmol, 1.00 eq.) in DMF (5 mL) at 0° C. was added sodium hydride (143 mg, 3.57 mmol, 60.0% purity, 2.00 eq.) and the mixture stirred at 0° C. for 30 minutes. After such time iodomethane (0.133 mL, 2.14 mmol, 1.20 eq.) in DMF (1 mL) was added and the mixture was stirred at 0° C. for a further 10 minutes. The reaction mixture was then diluted with water (50 mL) and extracted with ethyl acetate (40 mL×3) and the combined organic extracts were washed with brine (70 mL), dried over anhydrous sodium sulfate, filtered and concentrated under reduced pressure. The residue was purified by column chromatography (SiO₂, petroleum ether/ethyl acetate 0-30%) to give N-(4-bromo-2-methyl-pyrazol-3-yl)-N-methyl-benzamide (400 mg, 1.36 mmol, 76% yield) as a white solid. ¹H NMR (400 MHz, DMSO-d₆) δ=7.44 (s, 1H), 7.36-7.41 (m, 1H), 7.26-7.33 (m, 4H), 3.72 (s, 3H), 3.23 (s, 3H).

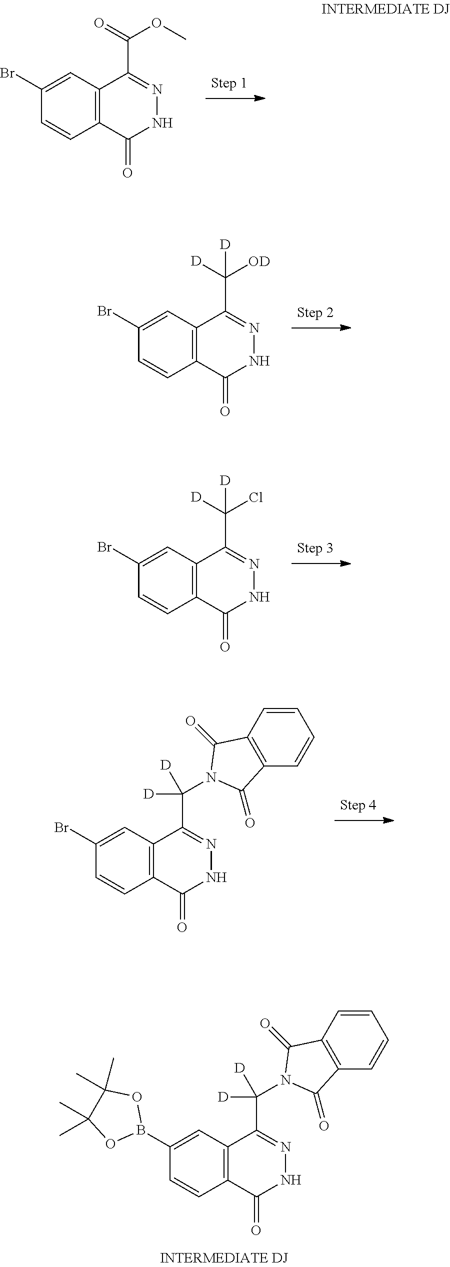

Step 1: To a stirred solution of methyl 7-bromo-4-oxo-3H-phthalazine-1-carboxylate (1.00 g, 3.53 mmol, 1.00 eq.), sodium borodeuteride (347 mg, 9.18 mmol, 2.60 eq.) in methanol-d4 (30 mL) at 0° C. was added calcium chloride (470 mg, 4.24 mmol, 1.20 eq.). The mixture then stirred at 0° C. for 3 hours then at 20° C. for 1 hour. After such time the reaction mixture was concentrated. The residue was diluted with water (30 mL), the pH adjusted to 5 with hydrochloric acid (1N, 5 mL) and the mixture filtered and the filter cake washed with water (5 mL×3) then triturated with ethyl alcohol (20 mL) to give 6-bromo-4-((hydroxy-d)methyl-d2)phthalazin-1 (2H)-one (463 mg, 1.61 mmol, 46% yield) as a white solid. LCMS [M+1]=259.0; ¹H NMR (400 MHz, DMSO-d₆) δ=12.66 (s, 1H), 8.31 (d, J=2.0 Hz, 1H), 8.17 (d, J=8.4 Hz, 1H), 8.02 (dd, J=2.0, 8.4 Hz, 1H), 5.53 (s, 1H).

Step 2: A mixture of 6-bromo-4-((hydroxy-d)methyl-d2)phthalazin-1 (2H)-one (463 mg, 1.61 mmol, 1.00 eq.) and thionyl chloride (10 mL) was stirred at 30° C. for 12 hours. After such time the mixture was concentrated and the residue dissolved in dichloromethane and concentrated 3 times (2 mL×3) to give 6-bromo-4-(chloromethyl-d2)phthalazin-1 (2H)-one (450 mg, 1.43 mmol, 88% yield) as a yellow solid. LCMS [M+1]⁺=277.0.

Step 3: To a solution of 6-bromo-4-(chloromethyl-d2)phthalazin-1 (2H)-one (450 mg, 1.63 mmol, 1.00 eq.) in DMF (3 mL) was added (1,3-dioxoisoindolin-2-yl)potassium (454 mg, 2.45 mmol, 1.50 eq.) and the mixture stirred at 90° C. for 2 hours. After such time the cooled reaction mixture was filtered and the collected solid was triturated with ethyl alcohol (5 mL), filtered and dried to give 2-((7-bromo-4-oxo-3,4-dihydrophthalazin-1-yl)methyl-d2)isoindoline-1,3-dione (300 mg, crude) as a white solid. LCMS [M+1]⁺=386.0, ¹H NMR (400 MHz, DMSO-d₆) δ=12.60 (s, 1H), 8.43 (d, J=1.6 Hz, 1H), 8.17 (d, J=8.4 Hz, 1H), 8.09-8.05 (m, 1H), 7.97-7.92 (m, 2H), 7.92-7.87 (m, 2H).

Step 4: A mixture of 2-((7-bromo-4-oxo-3,4-dihydrophthalazin-1-yl)methyl-d2)isoindoline-1,3-dione (200 mg, crude), 4,4,5,5-tetramethyl-2-(4,4,5,5-tetramethyl-1,3,2-dioxaborolan-2-yl)-1,3,2-dioxaborolane (197 mg, 0.78 mmol), Pd(dppf)Cl₂ (38 mg, 0.052 mmol) and potassium acetate (152 mg, 1.55 mmol) in dioxane (10 mL) was degassed with nitrogen. The mixture was stirred at 100° C. for 2 hours and after such time the mixture was concentrated and the residue triturated with methyl alcohol (3 mL), filtered and dried to give 2-((4-oxo-7-(4,4,5,5-tetramethyl-1,3,2-dioxaborolan-2-yl)-3,4-dihydrophthalazin-1-yl)methyl-d2)isoindoline-1,3-dione (200 mg, 0.303 mmol, 59% yield over 2 steps) as a white solid. LCMS [M+1]=352.1; ¹H NMR (400 MHz, DMSO-d₆) δ=12.70-12.28 (m, 1H), 8.35-8.25 (m, 2H), 8.13 (br s, 1H), 7.93 (br d, J=17.0 Hz, 4H), 1.34 (br s, 12H).

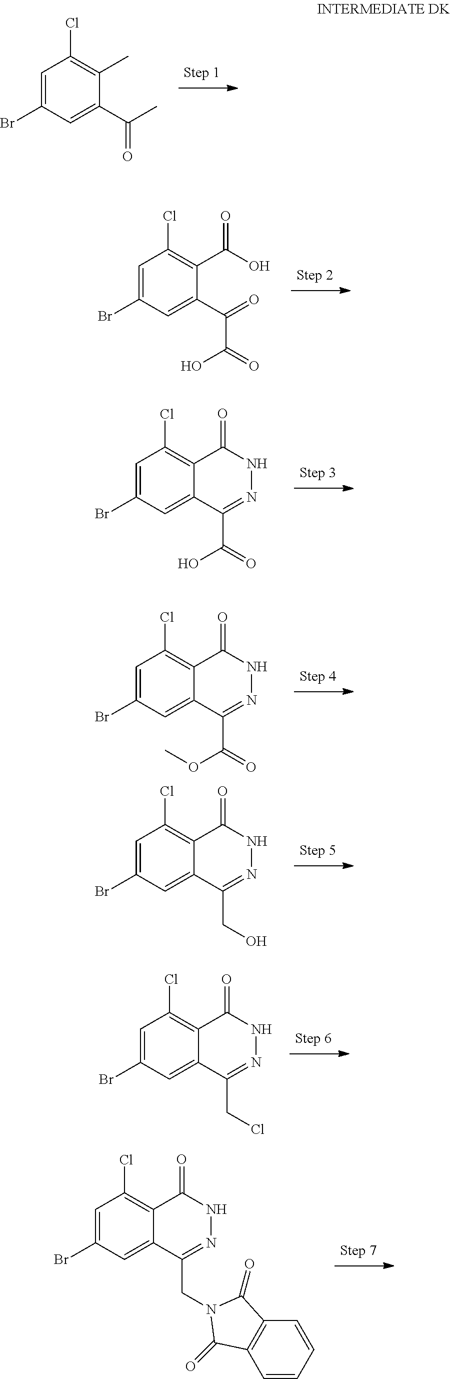

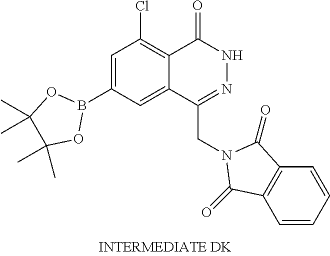 [SMILES: CC1(C)OB(c2cc(Cl)c3c(=O)[nH]nc(CN4C(=O)c5ccccc5C4=O)c3c2)OC1(C)C]

Step 1: To a solution of K₂CO₃ (44.7 g, 323 mmol) in water (500 mL) was added 1-(5-bromo-3-chloro-2-methylphenyl)ethan-1-one (40.0 g, 162 mmol) and warmed to 50° C. KMnO₄ was then added carefully in 10 batches (165 g, 1.04 mol) and the temperature maintained below 80° C. to avoid an uncontrolled exotherm. After completion of the addition the mixture was stirred at 60° C. for 6 hrs. After such time the mixture was cooled to 0° C. and quenched by the dropwise addition of saturated sodium sulfite solution (200 mL) while maintaining the temperature below 10° C. The mixture was then stirred for 30 min at 0° C. After such time the clear colorless mixture was filtrated with celatom and the filter cake was washed with water (100 mL) and the aqueous phase washed with MTBE (200 mL). The aqueous phase was then acidified to pH 2 by the addition of 3M HCl followed by extraction with ethyl acetate (300 mL×3). The combined organic phases were washed with brine (300 mL), dried with anhydrous Na₂SO₄, filtered and concentrated in vacuum to give compound 4-bromo-2-(carboxycarbonyl)-6-chlorobenzoic acid (20.0 g, 65.0 mmol, 36% yield) as white solid. LCMS [M−1]=306.8; ¹H NMR (400 MHz, DMSO-d₆) δ 12.7 (s, 1H), 8.10 (s, 1H), 8.00 (s, 1H).

Step 2: To a solution of compound 4-bromo-2-(carboxycarbonyl)-6-chlorobenzoic acid (20.0 g, 65.0 mmol) in EtOH (200 mL) was added NH₂NH₂·H₂O (4.30 g, 85.9 mmol) in one portion under N₂. The mixture was then stirred at 70° C. for 1 hr. After such time the cooled reaction mixture was filtered, washed and the solid dried in vacuum to give 7-bromo-5-chloro-4-oxo-3,4-dihydrophthalazine-1-carboxylic acid (14.0 g, 46.1 mmol, 71% yield) as white solid. LCMS [M+1]⁺=305.2; ¹H NMR (400 MHz, DMSO-d₆) δ 7.77 (d, J=2.0 Hz, 1H), 7.59 (d, J=2.0 Hz, 1H).

Step 3: To a mixture of 7-bromo-5-chloro-4-oxo-3,4-dihydrophthalazine-1-carboxylic acid (14.0 g, 46.1 mmol) in MeOH (250 mL) was added conc. H₂SO₄ (9.23 g, 92.2 mmol) in one portion under nitrogen. The mixture was then heated to 70° C. for 16 hrs then allowed to cool to ambient temperature, filtered and dried gave methyl 7-bromo-5-chloro-4-oxo-3,4-dihydrophthalazine-1-carboxylate (7.50 g, 23.6 mmol, 51% yield) as a white solid. ¹H NMR (400 MHz, DMSO-d₆) δ 13.2 (s, 1H), 6.68 (d, J=1.6 Hz, 1H), 8.18 (d, J=1.6 Hz, 1H).

Step 4: A solution of methyl 7-bromo-5-chloro-4-oxo-3,4-dihydrophthalazine-1-carboxylate (7.50 g, 23.6 mmol) in EtOH (70 mL) was added NaBH₄ (2.32 g, 61.4 mmol) at 0° C. followed by the careful, slow addition of CaCl₂) (3.15 g, 28.3 mmol) at 0° C. over 2 hours. The mixture was then allowed to warm to 15° C. and stirred for a further 2 hours. After such time the reaction was poured onto sat. NH₄Cl (100 mL) and the solid filtered, washed with water (10 mL) then EtOH (10 mL) and dried to give 6-bromo-8-chloro-4-(hydroxymethyl)phthalazin-1 (2H)-one (4.00 g, 13.8 mmol, 54% yield) as white solid. LCMS [M+1]⁺=291.0; ¹H NMR (400 MHz, DMSO-d₆) δ 12.6 (s, 1H), 8.24 (d, J=1.6 Hz, 1H), 8.14 (d, J=1.6 Hz, 1H), 5.61-5.58 (t, J=2H). Hz, 1H), 4.65-4.63 (d, J=8 Hz, 2H).

Step 5: A mixture of 6-bromo-8-chloro-4-(hydroxymethyl)phthalazin-1 (2H)-one (4.00 g, 13.8 mmol) and SOCl₂ (36.4 g, 306 mmol) was stirred at 65° C. for 1 hr. After such time the mixture was concentrated, and the crude residue triturated with MTBE (30 mL) at 25° C. for 30 min. The solid was then filtered and dried to give 6-bromo-8-chloro-4-(chloromethyl)phthalazin-1 (2H)-one (3.50 g, 11.4 mmol, 82% yield) as light yellow solid. LCMS [M+1]-=309.1; ¹H NMR (400 MHz, DMSO-d₆) δ 12.9 (s, 1H), 8.24 (s, 1H), 8.19 (s, 1H), 5.06 (s, 1H).

Step 6: To a mixture of potassium phthalimide (2.53 g, 13.6 mmol) in DMF (5 mL) was added a solution of 6-bromo-8-chloro-4-(chloromethyl)phthalazin-1 (2H)-one (3.50 g, 11.3 mmol) in DMF (35 mL) at 0° C. and the mixture stirred at 0° C. for 2 hrs. After such time the mixture was poured onto ice-water (200 mL), stirred for 30 min., filtered then the solid was dried and then triturated with MeOH (30 mL) at 15° C. for 30 min. The solid was filtered and dried to give 2-((7-bromo-5-chloro-4-oxo-3,4-dihydrophthalazin-1-yl)methyl)isoindoline-1,3-dione (1.30 g, 3.10 mmol, 27% yield) as light yellow solid. LCMS [M+1]⁺=420.0; ¹H NMR (400 MHz, DMSO-d₆) δ 12.5 (s, 1H), 8.36 (s, 1H), 8.19 (s, 1H), 7.97-7.94 (m, 2H), 7.92-7.89 (m, 2H), 5.14 (s, 2H).

Step 7: A mixture of 2-((7-bromo-5-chloro-4-oxo-3,4-dihydrophthalazin-1-yl)methyl)isoindoline-1,3-dione (1.30 g, 3.10 mmol), bis(pinacolato)diboron (1.20 g, 4.66 mmol) and potassium acetate (762 mg, 7.76 mmol) in dioxane (20 mL) was degassed with nitrogen. Then Pd(dppf)Cl₂ (114 mg, 0.16 mmol) was added and the mixture stirred at 70° C. for 2.5 hrs. After such time the mixture was cooled to room temperature, filtered and the concentrated residue triturated with MeOH (30 mL) at 15° C. for 30 min. The solid was then filtered, washed with MTB and dried to give 2-((5-chloro-4-oxo-7-(4,4,5,5-tetramethyl-1,3,2-dioxaborolan-2-yl)-3,4-dihydrophthalazin-1-yl)methyl)isoindoline-1,3-dione (910 mg, 1.95 mmol, 63% yield) as light yellow solid. LCMS [M+1]⁺=383.9; ¹H NMR (400 MHz, DMSO-d₆) δ 12.5 (s, 1H), 8.20 (s, 1H), 7.79 (s, 1H), 7.96-7.94 (m, 2H), 7.91-7.89 (m, 2H), 5.17 (s, 2H), 1.35 (s, 12H).

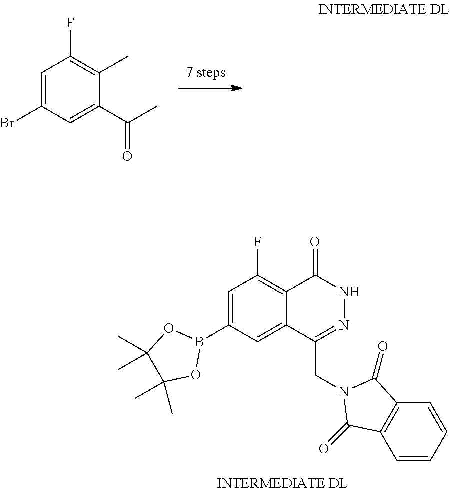 [SMILES: CC(=O)c1cc(Br)cc(F)c1C ; CC1(C)OB(c2cc(F)c3c(=O)[nH]nc(CN4C(=O)c5ccccc5C4=O)c3c2)OC1(C)C]

Following the same procedure for the synthesis of Intermediate DK, 2-((5-fluoro-4-oxo-7-(4,4,5,5-tetramethyl-1,3,2-dioxaborolan-2-yl)-3,4-dihydrophthalazin-1-yl)methyl)isoindoline-1,3-dione, Intermediate DL, was prepared as a white solid (200 mg, 0.42 mmol, 2.1% yield) in 7 steps from 1-(5-bromo-3-fluoro-2-methylphenyl)ethan-1-one. LCMS [M+1]⁺=368.1; ¹H NMR (400 MHz, DMSO-d₆) δ=12.51 (s, 1H), 8.09 (s, 1H), 7.96-7.89 (m, 4H), 7.40 (d, 1H), 5.17 (s, 2H), 1.36 (s, 12H).

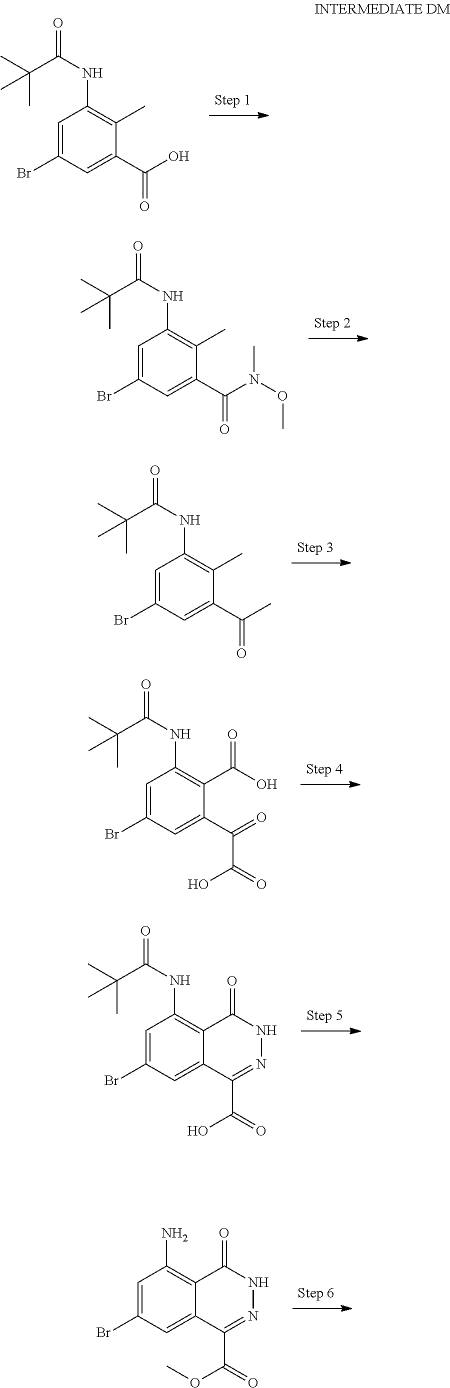

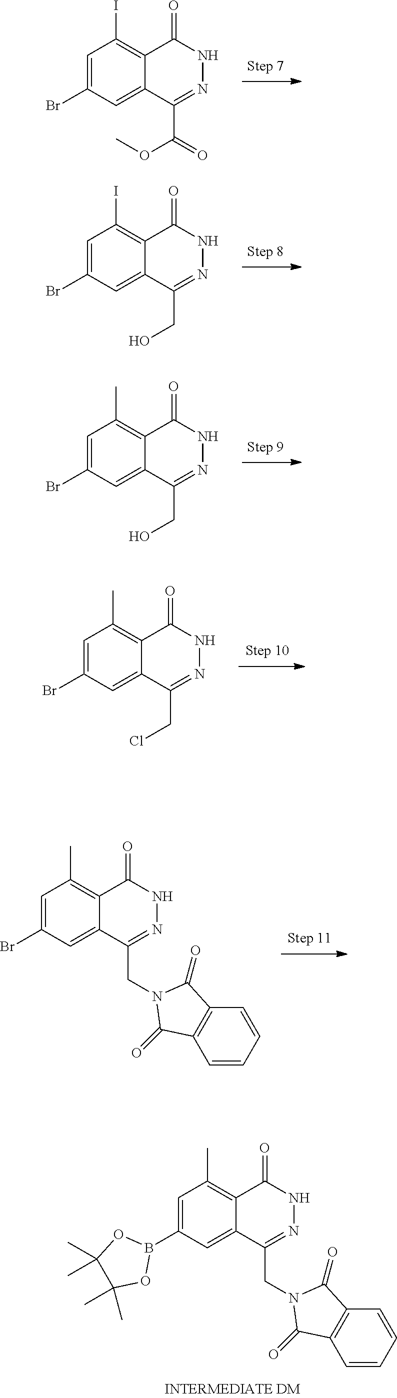

Step 1: To a solution of 5-bromo-2-methyl-3-pivalamidobenzoic acid (120 g, 382 mmol) in DMF (1.20 L) was added DIEA (98.7 g, 764 mmol, 133 mL), HATU (189 g, 497 mmol), followed by N,O-dimethylhydroxylamine (55.9 g, 573 mmol, HCl) at 20° C. The resulting solution was stirred at 20° C. for 2 hrs and after such time the reaction mixture was poured into ice water (5.0 L). The mixture was extracted with ethyl acetate (2.0 L×3) and the combined organic phase was washed with brine (1.0 L), dried with anhydrous Na₂SO₄, filtered and the filtrate was concentrated under reduced pressure to dry to afford 5-bromo-N-methoxy-N,2-dimethyl-3-pivalamidobenzamide (135 g, 378 mmol, 99% yield) as brown oil. ¹H NMR: 400 MHz, DMSO-d₆ δ 9.06 (s, 1H), 7.45 (d, J=2.0 Hz, 1H), 7.35 (d, J=2.0 Hz, 1H), 3.43 (s, 3H), 3.27 (s, 3H), 2.01 (s, 3H), 1.23 (s, 9H).

Step 2: To a solution of 5-bromo-N-methoxy-N,2-dimethyl-3-pivalamidobenzamide (135 g, 378 mmol) in THF (1.5 L) was added MeMgBr (3.0 M, 315 mL) at 0° C. The resulting solution was allowed to warm to 20° C. and stirred for 12 hrs. After such time an additional aliquot of MeMgBr (3 M, 63.0 mL) was added and the mixture stirred for a further 4 hrs. The mixture was then diluted with NH₄Cl (1.5 L), extracted with ethyl acetate (1.0 L×3) and the combined organic phases were washed with brine (1.0 L), dried with anhydrous Na₂SO₄ and filtered. The filtrate was concentrated to afford N-(3-acetyl-5-bromo-2-methylphenyl)pivalamide (115 g, 368 mmol, 98% yield) as yellow solid. ¹H NMR 400 MHz, DMSO-d₆ δ 9.10 (s, 1H), 7.75 (d, J=2.0 Hz, 1H), 7.51 (d, J=2.0 Hz, 1H), 2.55 (s, 3H), 2.10 (s, 3H), 1.23 (s, 9H).

Step 3: To a solution of N-(3-acetyl-5-bromo-2-methylphenyl)pivalamide (57.5 g, 184 mmol) in H₂O (600 mL) was added K₂CO₃ (50.9 g, 368 mmol) and KMnO₄ (204 g, 1.29 mol) at 50° C. The result solution was stirred at 50° C. for 17 hrs. After such time the reaction mixture was quenched by saturated sodium thiosulfate solution and filtered through diatomite. The pH was adjusted to 2 with 2N HCl and the mixture extracted with an ethyl acetate:THF 10:1 mixture (1.00 L×3), washed with brine (500 mL), dried over Na₂SO₄, filtered and concentrated under reduced pressure to give 4-bromo-2-(carboxycarbonyl)-6-pivalamidobenzoic acid (58.0 g, crude) as light yellow oil. ¹H NMR 400 MHz, DMSO-d₆ δ 9.87 (s, 1H), 8.63 (d, J=2.0 Hz, 1H), 7.54 (d, J=2.0 Hz, 1H), 1.27 (s, 9H).

Step 4: To a solution of 4-bromo-2-(carboxycarbonyl)-6-pivalamidobenzoic acid (110 g, 296 mmol) in EtOH (1.10 L) was added NH₂NH₂—H₂O (18.1 g, 355 mmol, 17.6 mL) and the solution was stirred at 75° C. for 3 hrs. After such time the reaction mixture was filtered and the filter cake dried to give 7-bromo-4-oxo-5-pivalamido-3,4-dihydrophthalazine-1-carboxylic acid (30.0 g, 81.5 mmol, 28% yield) as white solid. ¹H NMR 400 MHz, DMSO-d₆ δ 13.0 (s, 1H), 9.06 (d, J=2.0 Hz, 1H), 8.18 (d, J=2.0 Hz, 1H), 1.27 (s, 9H).

Step 5: To a solution of 7-bromo-4-oxo-5-pivalamido-3,4-dihydrophthalazine-1-carboxylic acid (30.0 g, 81.5 mmol) in MeOH (400 mL) was added a solution of HCl/MeOH (4 M, 400 mL). The reaction mixture was warmed to 70° C. and stirred for 36 hrs to form a yellow solid. The reaction was concentrated, diluted with water (100 mL) and the pH adjusted to pH 8 with 1 N NaOH, stirred for 0.5 hr then filtered. The filter cake was washed with water (50 mL) then EtOH (100 mL) and dried to give the crude product methyl 5-amino-7-bromo-4-oxo-3,4-dihydrophthalazine-1-carboxylate (20.0 g, crude) as a yellow solid.

Step 6: To a solution of methyl 5-amino-7-bromo-4-oxo-3,4-dihydrophthalazine-1-carboxylate (15.0 g, 50.3 mmol) in MeCN (500 mL) was added TosOH (34.6 g, 200 mmol) at 0° C. under N₂. To this solution was added a solution of NaNO₂ (8.68 g, 125 mmol) in H₂O (20 mL) and the mixture was stirred at 0° C. for 10 minutes then a solution of KI (25.0 g, 150 mmol) in H₂O (20 mL) was added dropwise. The mixture was stirred at 20° C. for 1 hr and the reaction was quenched by Na₂S₂O₃. The mixture was concentrated to remove the MeCN then diluted with water (200 mL) and filtered. The filter cake was washed with water (50 mL) then EtOH (100 mL) and dried to give the methyl 7-bromo-5-iodo-4-oxo-3,4-dihydrophthalazine-1-carboxylate (15.0 g, crude) as a yellow solid.

Step 7: In 8 batches a solution of methyl 7-bromo-5-iodo-4-oxo-3,4-dihydrophthalazine-1-carboxylate (4.00 g, 9.78 mmol) in EtOH (60 mL) was added NaBH₄ (740 mg, 19.6 mmol) in batches at 0° C. followed by the addition of CaCl₂ (1.30 g, 11.7 mmol) in batches at 0° C. The reaction was stirred at 20° C. for 1 hr. The 8 batches were then combined and quenched with NH₄Cl (200 mL). The mixture was concentrated to remove EtOH, diluted with water (200 mL) and then filtered and the filter cake was washed with water (100 mL) and dried. The residue was triturated in MeOH (200 mL) for 10 hrs, filtered and dried to give the 6-bromo-4-(hydroxymethyl)-8-iodophthalazin-1 (2H)-one (19.0 g, 38.9 mmol, 50% yield) as a yellow solid. ¹H NMR 400 MHz, DMSO-d₆ δ 12.64 (s, 1H), 8.56 (d, J=1.88 Hz, 1H), 8.29 (d, J=1.88 Hz, 1H), 5.62-5.54 (m, 1H), 4.62 (d, J=5.70 Hz, 2H).

Step 8: In three batches, to a mixture of 6-bromo-4-(hydroxymethyl)-8-iodophthalazin-1 (2H)-one (2.00 g, 5.25 mmol) in dioxane (40 mL) was added a solution of 2,4,6-trimethyl-1,3,5,2,4,6-trioxatriborinane in THF (3.67 mL, 13.1 mmol, 50% purity), CS₂CO₃ (4.28 g, 13.1 mmol) and Pd(dppf)Cl₂ (384 mg, 524 μmol). The reaction was stirred at 100° C. for 10 hrs. The three batches were combined and filtered through diatomite. The filtrate was concentrated and the residue purified by Prep-HPLC (Phenomenex luna C18 250×150 mm×15 μm; mobile phase: [water (0.1% TFA)-MeOH]; B %: 30%-60%, 20 min) to give the 6-bromo-4-(hydroxymethyl)-8-methylphthalazin-1 (2H)-one (1.1 g, 4.06 mmol, 26% yield) as a light yellow solid. LCMS [M+1]⁺=271; ¹H NMR 400 MHz, DMSO-d₆ δ 12.44 (s, 1H), 8.10 (s, 1H), 7.82 (s, 1H), 4.63 (s, 3H), 2.81 (s, 3H).

Step 9: A solution of 6-bromo-4-(hydroxymethyl)-8-methylphthalazin-1 (2H)-one (1.20 g, 4.46 mmol) in SOCl₂ (13 mL) was stirred at 70° C. for 2 hr. After such time the mixture was concentrated and the residue triturated in petroleum ether for 0.5 hr, filtered and dried to give 6-bromo-4-(chloromethyl)-8-methylphthalazin-1 (2H)-one (1.20 g, 4.17 mmol, 94% yield) as a light yellow solid. LCMS [M+1]⁺=289; ¹H NMR 400 MHz, DMSO-d₆ δ 12.70 (s, 1H), 8.08 (s, 1H), 7.88 (s, 1H), 5.03 (s, 2H), 2.81 (s, 3H).

Step 10: To a mixture of give 6-bromo-4-(chloromethyl)-8-methylphthalazin-1 (2H)-one (1.10 g, 3.83 mmol) in DMF (30 mL) was added potassium isoindoline-1,3-dione (850 mg, 4.59 mmol) in one portion at 0° C. under N₂. The mixture was stirred at 25° C. for 1 hr and after such time the mixture was slowly poured into ice water (100 mL) and the formed white solid was filtrated, washed with water and dried to give the crude product 2-((7-bromo-5-methyl-4-oxo-3,4-dihydrophthalazin-1-yl)methyl)isoindoline-1,3-dione (1.10 g, 2.32 mmol, 61% yield) as a white solid. LCMS [M+1]⁺=400; ¹H NMR 400 MHz, DMSO-d₆ δ 12.37 (s, 1H), 8.20 (d, J=1.32 Hz, 1H), 7.97-7.87 (m, 5H), 5.12 (s, 2H), 2.81 (s, 3H).

Step 11: 2-((7-bromo-5-methyl-4-oxo-3,4-dihydrophthalazin-1-yl)methyl)isoindoline-1,3-dione (1.10 g, 2.76 mmol), Pd(dppf)Cl₂ (202 mg, 276 μmol), KOAc (542 mg, 5.52 mmol) and bis(pinacolato)diboron (1.05 g, 4.14 mmol) in dioxane (20 mL) was de-gassed with nitrogen then heated at 80° C. for 10 hours. After such time the reaction was filtered through diatomite and the cake washed with MeOH (10 mL) and the filtrate concentrated. The residue was then triturated with MeOH (10 mL) for 1 hr, filtered and the filter cake washed with MeOH and dried to give 2-((5-methyl-4-oxo-7-(4,4,5,5-tetramethyl-1,3,2-dioxaborolan-2-yl)-3,4-dihydrophthalazin-1-yl)methyl)isoindoline-1,3-dione, Intermediate DM (510 mg, 1.15 mmol, 42% yield) as a gray solid. LCMS: Boronic acid [M+1]⁺=364; boronate ester [M+1]⁺=446). ¹H NMR 400 MHz, DMSO-d₆ δ 12.27 (s, 1H), 8.06 (s, 1H), 7.92-7.82 (m, 5H), 5.11 (s, 2H), 2.80 (s, 3H), 1.31 (s, 12H).

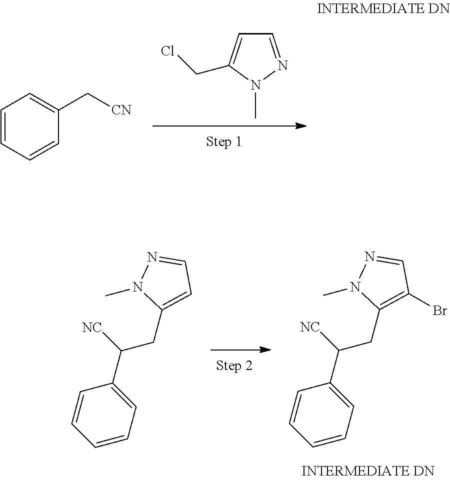

Step 1: To a solution of 5-(chloromethyl)-1-methyl-pyrazole (584 mg, 3.50 mmol, 1.00 eq) and 2-phenylacetonitrile (819 mg, 6.99 mmol, 2.00 eq.) in DMF (10 mL) was added potassium carbonate (966 mg, 6.99 mmol, 2.00 eq.). The mixture was stirred at 120° C. for 4 hours then and concentrated under reduced pressure. The residue was purified by column chromatography (SiO₂, petroleum ether/ethyl acetate 10-50%) to give 3-(2-methylpyrazol-3-yl)-2-phenyl-propanenitrile (300 mg, 1.42 mmol, 41% yield) as a brown oil. LCMS [M+1]⁺=212.0, ¹H NMR (400 MHz, CDCl₃) δ=7.35-7.22 (m, 4H), 7.18-7.10 (m, 2H), 6.11 (d, J=1.6 Hz, 1H), 3.98 (t, J=6.8 Hz, 1H), 3.41 (s, 3H), 3.26-3.18 (m, 1H), 3.16-3.05 (m, 1H).

Step 2: To a mixture of 3-(2-methylpyrazol-3-yl)-2-phenyl-propanenitrile (160 mg, 0.76 mmol, 1.00 eq.) in dry acetonitrile (2.0 mL) was added NBS (121 mg, 0.68 mmol, 0.90 eq.) in several portions. The mixture was stirred at 15° C. for 2 hours. After such time ethyl acetate (40 mL) and water (40 mL) were added and the layers separated. The aqueous phase was extracted with ethyl acetate (30 mL×2) and the combined organic extracts were washed with brine (50 mL), dried over anhydrous sodium sulfate, filtered, and concentrated in vacuum. The residue was purified by prep-HPLC (Phenomenex Gemini-NX C18 75×30 mm×3 μm; mobile phase: [water (10 mM NH₄HCO₃)-ACN]; B %: 30%-60%, 8 min) to give 3-(4-bromo-2-methyl-pyrazol-3-yl)-2-phenyl-propanenitrile (90.0 mg, 0.31 mmol, 41% yield) as a yellow oil. LCMS [M+1]⁺=289.8; ¹H NMR (400 MHz, CDCl₃) δ=7.37 (s, 1H), 7.35-7.26 (m, 3H), 7.20-7.14 (m, 2H), 4.04 (t, 1=7.6 Hz, 1H), 3.40 (s, 3H), 3.30 (dd, J=7.2, 14.8 Hz, 1H), 3.07 (dd, J=8.0, 14.8 Hz, 1H).

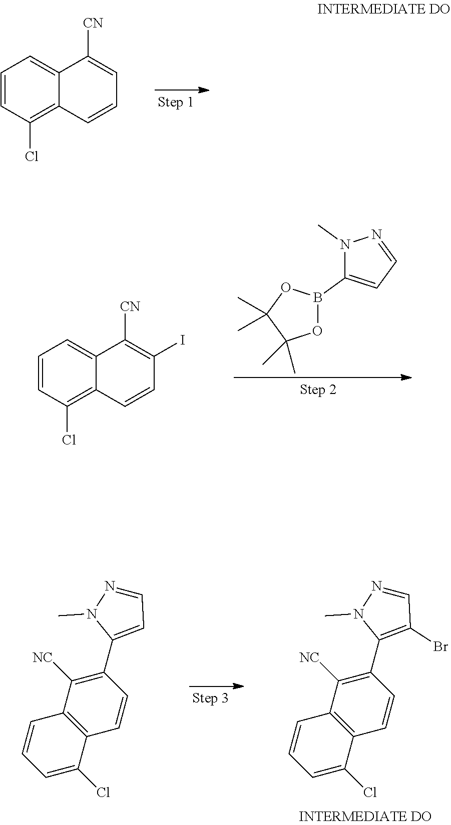 [SMILES: N#Cc1cccc2c(Cl)cccc12 ; N#Cc1c(I)ccc2c(Cl)cccc12 ; Cn1nccc1B1OC(C)(C)C(C)(C)O1 ; Cn1nccc1-c1ccc2c(Cl)cccc2c1C#N ; Cn1ncc(Br)c1-c1ccc2c(Cl)cccc2c1C#N]

Step 1: n-Butyllithium (2.5 M in hexane, 959 μL, 1.50 eq.) was added dropwise over 5 minutes to a solution of 2,2,6,6-tetramethylpiperidine (2.40 mmol, 407 μL, 1.50 eq.) in THF (3 mL) maintained at 0° C. After 30 minutes, the reaction mixture was cooled to −78° C. and a solution of 5-chloronaphthalene-1-carbonitrile (300 mg, 1.60 mmol, 1.00 eq.) in THF (1.0 mL) was added dropwise over 10 minutes. The resulting dark solution was maintained at −78° C. for 2 hours. A solution of iodine (609 mg, 2.40 mmol, 1.50 eq.) in THF (3 mL) was then added dropwise over 10 minutes. The reaction mixture was maintained at −78° C. for 2 hours then allowed to warm to 20° C. for 3 hours. The reaction mixture was quenched with water (1 mL) and the resulting mixture diluted with ethyl acetate (150 mL). The mixture was washed successively with saturated aqueous sodium thiosulfate (3×150 mL), 1 M HCl (2×150 mL), and brine (1×150 mL). The organic layer was dried over anhydrous sodium sulfate, filtered, and concentrated. The residue was purified by column chromatography (SiO₂, petroleum ether:ethyl acetate 5%) to give 5-chloro-2-iodo-1-naphthonitrile (180 mg, 574 μmol, 36% yield) as a yellow solid. GCMS [M+H]⁺=312.9; ¹H NMR (400 MHz, CDCl₃) δ=8.55 (d, J=8.8 Hz, 1H), 8.01-7.97 (m, 1H), 7.73-7.69 (m, 1H), 7.67-7.64 (m, 1H), 7.63-7.58 (m, 1H).

Step 2: A mixture of 1-methyl-5-(4,4,5,5-tetramethyl-1,3,2-dioxaborolan-2-yl)-1H-pyrazole (104 mg, 498 μmol, 1.30 eq.), 5-chloro-2-iodo-1-naphthonitrile (120 mg, 383 μmol, 1.00 eq.), Pd(dtbpf)Cl₂ (25 mg, 38 μmol, 0.10 eq.) and sodium carbonate (81 mg, 766 μmol, 2.00 eq.) in the dioxane (3 mL) and water (0.6 mL) was degassed with nitrogen then stirred at 80° C. for 1 hour. The mixture was then concentrated and the residue purified by prep-TLC (SiO₂, petroleum ether:ethyl acetate 30%) to give 5-chloro-2-(2-methylpyrazol-3-yl)naphthalene-1-carbonitrile (90 mg, 336 μmol, 87% yield) as a yellow solid. LCMS [M+1]⁺=268.2; ¹H NMR (400 MHz, CDCl₃) δ=8.62 (d, J=8.8 Hz, 1H), 8.30 (d, J=8.4 Hz, 1H), 7.81-7.77 (m, 1H), 7.73-7.67 (m, 1H), 7.67-7.63 (m, 2H), 6.61 (d, J=2.0 Hz, 1H), 3.91 (s, 3H).

Step 3: To a solution of 5-chloro-2-(2-methylpyrazol-3-yl)naphthalene-1-carbonitrile (170 mg, 635 μmol, 1.00 eq.) in acetonitrile (3 mL) was added NBS (124 mg, 699 μmol, 1.10 eq.). The mixture was stirred at 35° C. for 2 hours then concentrated and the residue was purified by prep-TLC (SiO₂, petroleum ether/ethyl acetate 30%) to give 2-(4-bromo-1-methyl-1H-pyrazol-5-yl)-5-chloro-1-naphthonitrile, Intermediate DO (130 mg, 375 μmol, 59% yield) as a white solid. LCMS [M+1]⁺=347.8, ¹H NMR (400 MHz, CDCl₃) δ=8.67 (dd, J=0.8, 8.8 Hz, 1H), 8.31 (d, J=8.4 Hz, 1H), 7.83 (dd, J=1.2, 7.6 Hz, 1H), 7.75-7.69 (m, 1H), 7.66 (s, 1H), 7.64 (d, J=8.8 Hz, 1H), 3.86 (s, 3H).

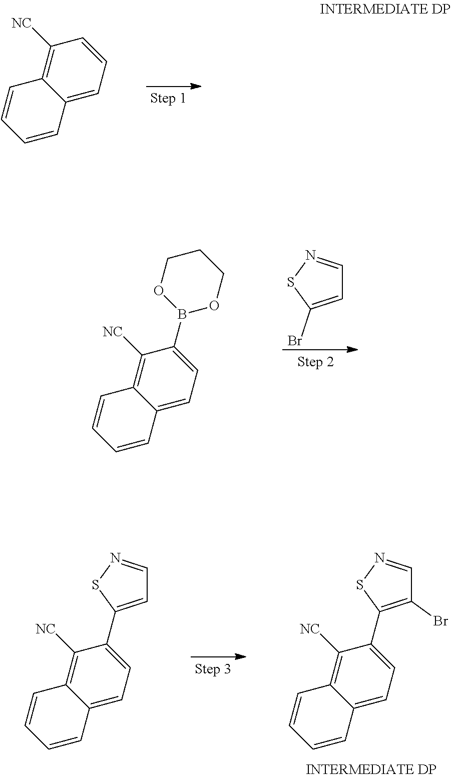 [SMILES: N#Cc1cccc2ccccc12 ; N#Cc1c(B2OCCCO2)ccc2ccccc12 ; Brc1ccns1 ; N#Cc1c(-c2ccns2)ccc2ccccc12 ; N#Cc1c(-c2sncc2Br)ccc2ccccc12]

Step 1: To a solution of 2,2,6,6-tetramethylpiperidine (553 mg, 3.92 mmol, 0.67 mL, 1.20 eq.) in THF (7 mL) was added n-butyl lithium (2.50 M, 1.57 mL, 1.20 eq.) at −10° C. under a nitrogen atmosphere. The mixture was stirred for 10 minutes, cooled to −65° C. and triisopropyl borate (859 mg, 4.57 mmol, 1.05 mL, 1.40 eq.) was added. After 5 minutes, a solution of 1-naphthonitrile (500 mg, 3.26 mmol, 1.00 eq.) in THF (3 mL) was added in a dropwise fashion and the reaction was then allowed to warm slowly to 25° C. and then stirred for 16 hours. After such time acetic acid (392 mg, 6.53 mmol, 0.37 mL, 2.00 eq.) was added followed by the addition of propane-1,3-diol (994 mg, 13.1 mmol, 0.95 mL, 4.00 eq.) then the mixture was stirred at 25° C. for 1 hour. The reaction was then quenched by the addition of saturated ammonium chloride solution (20 mL) and then diluted with water (10 mL) and extracted with ethyl acetate (30 mL×2). The combined organic layers were dried over anhydrous sodium sulfate, filtered and concentrated under reduced pressure to give 2-(1,3,2-dioxaborinan-2-yl)-1-naphthonitrile (600 mg, 2.53 mmol, 78% yield) as a light-yellow solid. ¹H NMR (400 MHz, CDCl₃) δ=8.40 (d, J=8.0 Hz, 1H), 8.04-8.00 (m, 1H), 7.90 (d, J=8.0 Hz, 2H), 7.71-7.60 (m, 2H), 4.30 (t, J=5.6 Hz, 4H), 2.17 (quin, J=5.6 Hz, 2H).

Step 2: To a solution of 5-bromoisothiazole (150 mg, 0.915 mmol, 1.00 eq.) and 2-(1,3,2-dioxaborinan-2-yl)-1-naphthonitrile (217 mg, 0.915 mmol, 1.00 eq.) in toluene (8 mL) and ethyl alcohol (0.8 mL) were added aqueous potassium carbonate (2.00 M, 0.915 mL, 2.00 eq.) and Pd(PPh₃)₄ (106 mg, 0.091 mmol, 0.10 eq.) at 20° C. under a nitrogen atmosphere. The mixture was stirred at 100° C. for 16 hours, concentrated to dryness and the residue was purified by column chromatography (SiO₂, petroleum ether:ethyl acetate 10-15%) to give 2-(isothiazol-5-yl)-1-naphthonitrile (200 mg, 0.85 mmol, 93% yield) as a light yellow solid. ¹H NMR (400 MHz, CDCl₃) δ=8.62 (d, J=1.6 Hz, 1H), 8.37 (d, J=8.8 Hz, 1H), 8.15 (d, J=8.8 Hz, 1H), 7.97 (d, J=8.0 Hz, 1H), 7.89 (d, J=2.0 Hz, 1H), 7.79 (dt, J=1.2, 7.6 Hz, 1H), 7.74-7.66 (m, 2H).

Step 3: To a solution of 2-(isothiazol-5-yl)-1-naphthonitrile (100 mg, 0.42 mmol, 1.00 eq.) in acetonitrile (2 mL) was added N-bromo-succinimide (753 mg, 4.23 mmol, 10.0 eq.) at 20° C. and the mixture was stirred at 100° C. for 48 hours in a sealed tube. The mixture was then concentrated under reduced pressure and the residue diluted with ethyl acetate (30 mL) and washed with water (30 mL×2), dried over anhydrous sodium sulfate, filtered and concentrated. The residue was then purified by prep-HPLC (Waters Xbridge BEH C18 100×30 mm 10 μm; mobile phase: [water (10 mM NH₄HCO₃)-ACN]; B %: 45%-75%, 8 min) to give 2-(4-bromoisothiazol-5-yl)-1-naphthonitrile (50 mg, 0.16 mmol, 38% yield) as a white solid. ¹H NMR (400 MHz, CDCl₃) δ=8.51 (s, 1H), 8.37 (d, J=8.8 Hz, 1H), 8.19 (d, J=8.8 Hz, 1H), 8.02 (d, J=8.0 Hz, 1H), 7.81 (dt, J=1.2, 7.6 Hz, 1H), 7.77-7.69 (m, 1H), 7.59 (d, J=8.4 Hz, 1H).

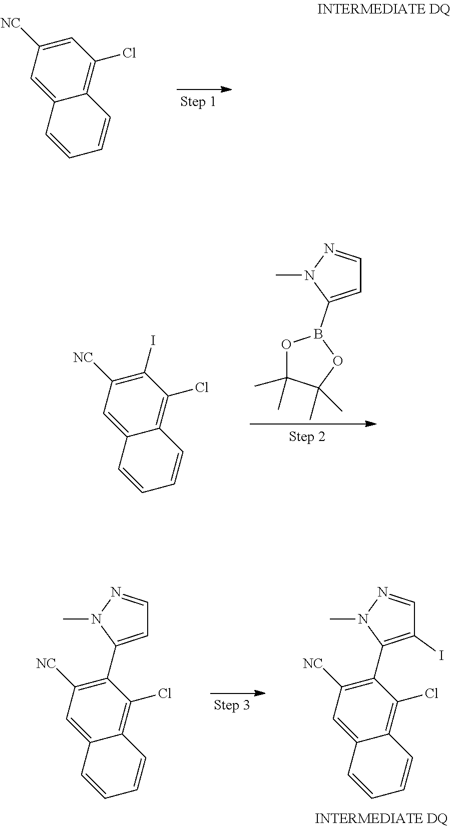 [SMILES: N#Cc1cc(Cl)c2ccccc2c1 ; N#Cc1cc2ccccc2c(Cl)c1I ; Cn1nccc1B1OC(C)(C)C(C)(C)O1 ; Cn1nccc1-c1c(C#N)cc2ccccc2c1Cl ; Cn1ncc(I)c1-c1c(C#N)cc2ccccc2c1Cl]

Step 1: To a solution of LDA (2.00 M, 0.587 mL, 1.10 eq.) in THF (10 mL) was added in a dropwise fashion a solution of 4-chloro-2-naphthonitrile (200 mg, 1.07 mmol, 1.00 eq) in THF (5 mL) at −78° C. Then the mixture was stirred at −78° C. for 1 hour. After such time a solution of iodine (285 mg, 1.12 mmol, 1.05 eq.) in THF (2 mL) was added dropwise at −78° C. The mixture was then allowed to warm to room temperature and stirred at 20° C. for 2 hours. After such time the reaction mixture was quenched by adding saturated ammonium chloride solution (15 mL) and saturated sodium hyposulfite solution (10 mL×3). The mixture was then extracted with ethyl acetate (20 mL×2) and the combined organic layers were dried over anhydrous sodium sulfate, filtered, concentrated and the residue purified by flash chromatography (SiO₂, petroleum ether:ethyl acetate 0-5%) to give 4-chloro-3-iodo-2-naphthonitrile (200 mg, 0.606 mmol, 30% yield) as a white solid. ¹H NMR (400 MHz, CDCl₃) δ=8.35 (d, J=8.4 Hz, 1H), 8.13 (s, 1H), 7.93-7.87 (m, 1H), 7.77 (ddd, J=1.2, 7.2, 8.4 Hz, 1H), 7.72-7.65 (m, 1H).

Step 2: To a solution of 4-chloro-3-iodo-2-naphthonitrile (320 mg, 1.02 mmol, 1.00 eq.) and 1-methyl-5-(4,4,5,5-tetramethyl-1,3,2-dioxaborolan-2-yl)pyrazole (319 mg, 1.53 mmol, 1.50 eq.) in dioxane (30 mL) and water (6 mL) was added potassium carbonate (283 mg, 2.04 mmol, 2.00 eq.) and Pd(dppf)Cl₂ (75 mg, 0.102 mmol, 0.10 eq.) at 25° C. The mixture was degassed with nitrogen then stirred at 100° C. for 16 hours. The reaction mixture was then quenched with water (20 mL) and extracted with ethyl acetate (30 mL×4). The combined organic layers were washed with brine (25 mL×2), dried over anhydrous sodium sulfate, filtered, concentrated and the residue purified by flash chromatography (SiO₂, petroleum ether:ethyl acetate 0-5%) to give 4-chloro-3-(1-methyl-1H-pyrazol-5-yl)-2-naphthonitrile (50 mg, 0.178 mmol, 30% yield) as a yellow solid. LCMS [M+1]⁺=268.0/270.0; ¹H NMR (400 MHz, CDCl₃) δ=8.48-8.39 (m, 1H), 8.34-8.27 (m, 1H), 8.04-7.95 (m, 1H), 7.90-7.81 (m, 1H), 7.78 (br t, J=7.6 Hz, 1H), 7.70-7.63 (m, 1H), 6.52-6.43 (m, 1H), 3.80-3.72 (m, 3H).

Step 3: To a solution of 4-chloro-3-(1-methyl-1H-pyrazol-5-yl)-2-naphthonitrile (100 mg, 0.374 mmol, 1.00 eq.) in acetonitrile (10 mL) was added N-iodosuccinimide (504 mg, 2.24 mmol, 6.00 eq.) at 25° C. and the mixture was stirred at 80° C. for 16 hours. After such time the reaction mixture was quenched with water (2 mL) at 0° C., and then extracted with ethyl acetate (3 mL×3). The combined organic layers were washed with brine (5 mL 2), dried over anhydrous sodium sulfate, filtered and concentrated. The formed residue was purified by prep-TLC (SiO₂, petroleum ether:ethyl acetate 30%) to give 4-chloro-3-(4-iodo-1-methyl-1H-pyrazol-5-yl)-2-naphthonitrile (50 mg, 0.121 mmol, 32% yield) as a white solid. LCMS [M+1]⁺=393.9/395.9; ¹H NMR (400 MHz, CDCl₃) δ=8.46 (d, J=8.4 Hz, 1H), 8.35 (s, 1H), 8.04 (d, J=8.0 Hz, 1H), 7.92-7.86 (m, 1H), 7.84-7.79 (m, 1H), 7.71 (s, 1H), 3.81 (s, 3H).

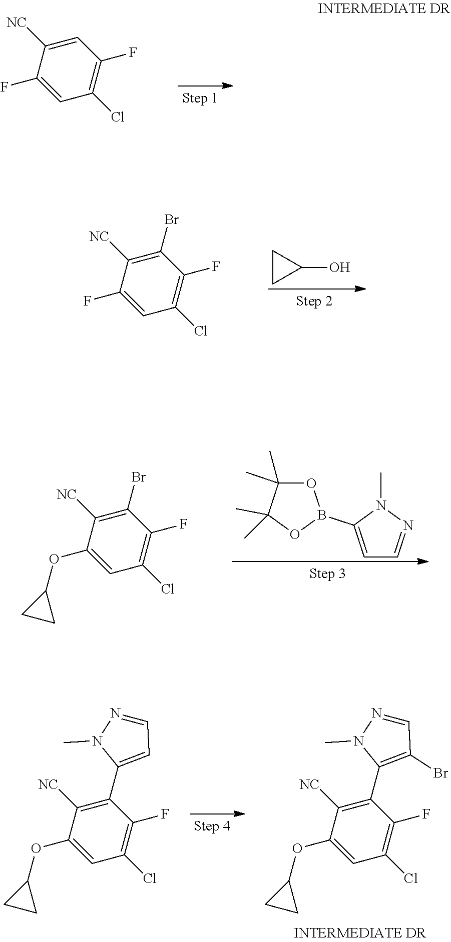

Step 1: A mixture of 4-chloro-2,5-difluoro-benzonitrile (2.00 g, 11.5 mmol, 1.00 eq.), N-bromosuccinimide (4.10 g, 23.1 mmol, 2.00 eq.), palladium acetate (259 mg, 1.15 mmol, 0.10 eq.) and p-toluene sulphonic acid (992 mg, 5.76 mmol, 0.50 eq.) in dichloroethane (50 mL) was degassed with nitrogen then stirred at 75° C. for 12 hours. After such time the cooled mixture was extracted with dichloromethane (50 mL×3), washed with brine (50 mL), dried over anhydrous sodium sulfate, filtered, concentrated under reduced pressure and the residue purified by column chromatography (SiO₂, petroleum ether:ethyl acetate 0-3%) to give 2-bromo-4-chloro-3,6-difluoro-benzonitrile (1.10 g, 4.36 mmol, 38% yield) as a white solid. ¹H NMR (400 MHz, CDCl₃) δ=7.38-7.31 (m, 1H).

Step 2: A mixture of 2-bromo-4-chloro-3,6-difluoro-benzonitrile (1.10 g, 4.36 mmol, 1.00 eq.), cyclopropanol (380 mg, 6.54 mmol, 1.50 eq.) and potassium carbonate (1.51 g, 10.9 mmol, 2.50 eq.) in DMF (10 mL) was degassed with nitrogen then stirred at 75° C. for 2 hours. After such time the mixture was concentrated and the residue was purified by prep-TLC (SiO₂, petroleum ether:ethyl acetate 7%) to give 2-bromo-4-chloro-6-(cyclopropoxy)-3-fluoro-benzonitrile (600 mg, 2.07 mmol, 47% yield) as a white solid. ¹H NMR (400 MHz, CDCl₃) δ=7.36 (d, J=5.6 Hz, 1H), 3.88-3.79 (m, 1H), 0.91 (d, J=4.8 Hz, 4H).

Step 3: A mixture of 1-methyl-5-(4,4,5,5-tetramethyl-1,3,2-dioxaborolan-2-yl)pyrazole (1.29 g, 6.20 mmol, 3.00 eq.), 2-bromo-4-chloro-6-(cyclopropoxy)-3-fluoro-benzonitrile (600 mg, 2.07 mmol, 1.00 eq.), aqueous sodium bicarbonate (694 mg, 8.26 mmol, 0.321 mL, 4.00 eq.), di-tert-butyl(cyclopentyl)phosphane;dichloropalladium-iron (135 mg, 0.207 mmol, 0.10 eq.) in dioxane (20 mL) and water (4 mL) was degassed with nitrogen and the mixture was stirred at 80° C. for 16 hours. After such time the mixture was concentrated and the residue purified by column chromatography (SiO₂, petroleum ether:ethyl acetate 5-20%) to give 4-chloro-6-(cyclopropoxy)-3-fluoro-2-(2-methylpyrazol-3-yl)benzonitrile (180 mg, 0.524 mmol, 25% yield) as a yellow solid. LCMS [M+1]+=292.1; ¹H NMR (400 MHz, CDCl₃) δ=7.62 (d, 0.1=2.0 Hz, 1H), 7.49 (d, J=6.0 Hz, 1H), 6.50 (d, J=2.0 Hz, 1H), 3.92-3.85 (m, 1H), 3.81 (d, J=1.2 Hz, 3H), 0.96-0.92 (m, 4H).

Step 4: A mixture of 4-chloro-6-(cyclopropoxy)-3-fluoro-2-(2-methylpyrazol-3-yl)benzonitrile (180 mg, 0.617 mmol, 1.00 eq) and N-bromosuccinimide (220 mg, 1.23 mmol, 2.00 eq.) in acetonitrile (10 mL) was stirred at 40° C. for 2 hours under a nitrogen atmosphere. After such time the mixture was concentrated and the residue was purified by prep-TLC (SiO₂, petroleum ether:ethyl acetate 20%) to give 2-(4-bromo-2-methyl-pyrazol-3-yl)-4-chloro-6-(cyclopropoxy)-3-fluoro-benzonitrile (170 mg, 0.455 mmol, 74% yield) as a white solid. LCMS [M+1]⁺=371.8; ¹H NMR (400 MHz, CDCl₃) δ=7.61 (s, 1H), 7.55 (d, J=6.0 Hz, 1H), 3.93-3.85 (m, 1H), 3.80 (s, 4H), 0.97-0.94 (m, 4H).

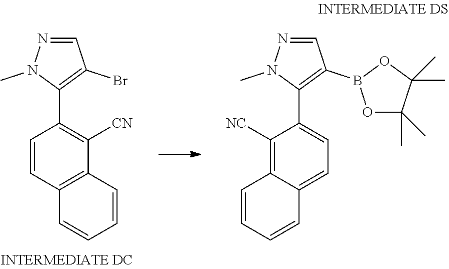

A mixture of 2-(4-bromo-2-methyl-pyrazol-3-yl)naphthalene-1-carbonitrile (150 mg, 0.48 mmol, 1.00 eq.), 4,4,5,5-tetramethyl-2-(4,4,5,5-tetramethyl-1,3,2-dioxaborolan-2-yl)-1,3,2-dioxaborolane (134 mg, 0.528 mmol, 1.10 eq.), potassium acetate (141 mg, 1.44 mmol, 3.00 eq.) and di-tert-butyl(cyclopentyl)phosphane;dichloropalladium-iron (31.3 mg, 0.048 mmol, 0.10 eq.) in dioxane (3 mL) was degassed with nitrogen and then stirred at 80° C. for 2 hours. After such time the reaction mixture was concentrated under reduced pressure to give 2-[2-methyl-4-(4,4,5,5-tetramethyl-1,3,2-dioxaborolan-2-yl)pyrazol-3-yl]naphthalene-1-carbonitrile (160 mg, crude) as brown liquid which used into the next step without further purification. LCMS [M+1]⁺=360.2.

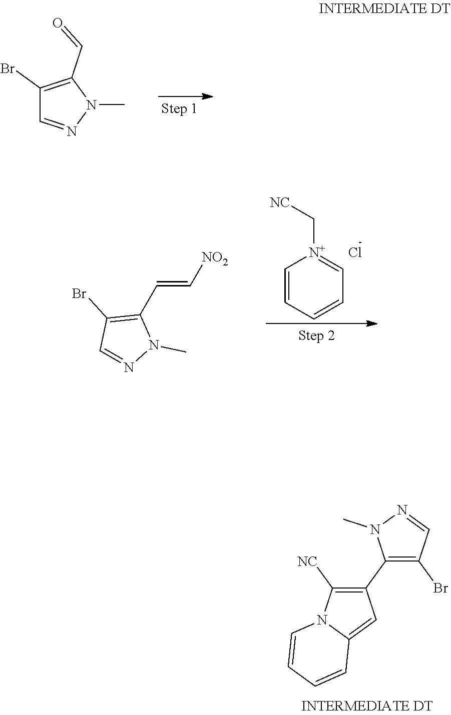

Step 1: To a solution of 4-bromo-2-methyl-pyrazole-3-carbaldehyde (1.00 g, 5.29 mmol, 1.00 eq.) and nitromethane (420 mg, 6.88 mmol, 0.37 mL, 1.30 eq.) in methanol (10 mL) was added in a dropwise fashion a solution of sodium hydroxide (466 mg, 11.6 mmol, 2.20 eq.) in water (1 mL) at 0° C. The reaction mixture was then stirred at 0° C. for 0.5 hour. After such time the reaction mixture was quenched by addition of HCl (1.00 M, 5 mL), filtered and the filtrate concentrated under reduced pressure to give 4-bromo-1-methyl-5-[(E)-2-nitrovinyl]pyrazole (627 mg, crude) as a yellow solid which used into the next step without further purification. ¹H NMR (400 MHz, CDCl₃) δ=8.10 (d, J=13.6 Hz, 1H), 7.93 (d, J=13.6 Hz, 1H), 7.57 (s, 1H), 4.03 (s, 3H).

Step 2: A suspension of 2-pyridin-1-ium-1-ylacetonitrile chloride (627 mg) and 4 Å MS (1.00 g, o.215 mmol) in dichloroethane (30 mL) was cooled to 0° C. then 2,6-lutidine (1.45 g, 13.5 mmol, 1.57 mL, 5.00 eq.) was added. After stirring for 15 minutes, 4-bromo-1-methyl-5-[(E)-2-nitrovinyl]pyrazole (627 mg, 2.70 mmol, 1.00 eq.) was added, followed by the addition of cupric acetate (736 mg, 4.05 mmol, 1.50 eq.). This mixture was then stirred at 0° C. for 15 minutes then warmed to 25° C. and stirred at 25° C. for 5 hours. After such time the reaction mixture was diluted with water (300 mL) and extracted with EtOAc (100 mL×3). The combined organic layers were dried over anhydrous sodium sulfate, filtered, concentrated and the residue was purified by column chromatography (SiO₂, petroleum ether:ethyl acetate 10-20%) to give 2-(4-bromo-2-methyl-pyrazol-3-yl)indolizine-3-carbonitrile (380 mg, 1.26 mmol, 47% yield) as a yellow solid. LCMS [M+1]⁺=301.0; ¹H NMR (400 MHz, CDCl₃) δ=8.34 (d, J=6.0 Hz, 1H), 7.62-7.53 (m, 2H), 7.18-7.11 (m, 1H), 6.96 (dt, J=1.2, 6.8 Hz, 1H), 6.62 (s, 11H), 3.91 (s, 3H).

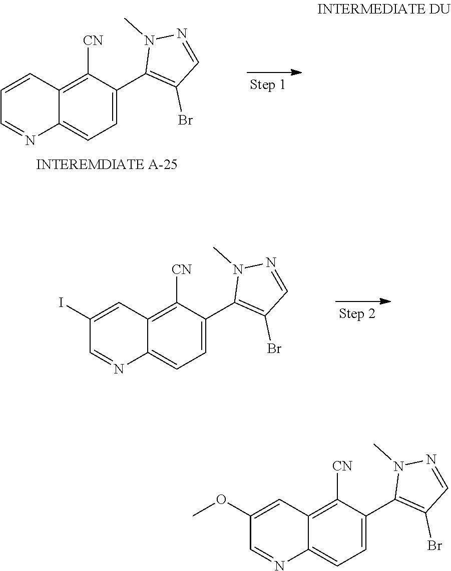 [SMILES: Cn1ncc(Br)c1-c1ccc2ncccc2c1C#N ; Cn1ncc(Br)c1-c1ccc2ncc(I)cc2c1C#N ; COc1cnc2ccc(-c3c(Br)cnn3C)c(C#N)c2c1]

Step 1: A mixture of 6-(4-bromo-1-methyl-1H-pyrazol-5-yl)quinoline-5-carbonitrile, Intermediate A-25 (120 mg, 0.38 mmol, 1.00 eq.), N-iodosuccinimide (517 mg, 2.30 mmol, 6.00 eq.) in acetic acid (5 mL) was stirred at 80° C. for 48 hours under a nitrogen atmosphere. The mixture was then concentrated and to the residue was added saturated sodium sulfite solution (10 mL). The mixture was then extracted with ethyl acetate (5 mL) and the organic phase was washed with brine (10 mL), dried over anhydrous sodium sulfate and concentrated. The residue was purified by prep-TLC (SiO₂, petroleum ether:ethyl acetate 50%) to give 6-(4-bromo-1-methyl-1H-pyrazol-5-yl)-3-iodoquinoline-5-carbonitrile (38 mg, 0.086 mmol, 22% yield) as a white solid. LCMS [M+1]⁺=441.1; ¹H NMR (400 MHz, CDCl₃) δ=9.26 (d, J=2.0 Hz, 1H), 9.03 (dd, J=0.8, 2.0 Hz, 1H), 8.43 (dd, J=0.8, 8.8 Hz, 1H), 7.77 (d, J=8.8 Hz, 1H), 7.67 (s, 1H), 3.87 (s, 3H).

Step 2: A mixture of 6-(4-bromo-1-methyl-1H-pyrazol-5-yl)-3-iodoquinoline-5-carbonitrile (35 mg, 0.080 mmol, 1.00 eq.), sodium methoxide (13 mg, 0.24 mmol, 3.00 eq.), cuprous iodide (1.5 mg, 0.008 mmol, 0.10 eq.) in methanol (1 mL) was degassed with nitrogen then stirred at 105° C. for 16 hours. After such time the mixture was filtered and the filtrate concentrated and the residue was purified by prep-TLC (SiO₂, petroleum ether:ethyl acetate 50%) to give 6-(4-bromo-1-methyl-1H-pyrazol-5-yl)-3-methoxyquinoline-5-carbonitrile (12 mg, 0.035 mmol, 44% yield) as a white solid. LCMS [M+1]⁺=345.1; ¹H NMR (400 MHz, CDCl₃) δ=8.86 (d, J=2.8 Hz, 1H), 8.40 (d, J=8.4 Hz, 1H), 7.76 (d, J=2.8 Hz, 1H), 7.66 (s, 1H), 7.58 (d, J=8.4 Hz, 1H), 4.07 (s, 3H), 3.87 (s, 3H).

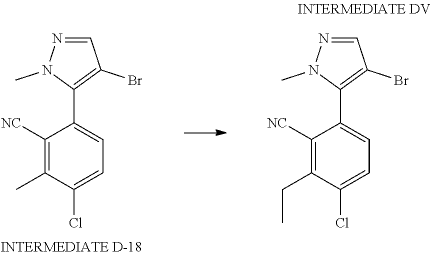 [SMILES: Cc1c(Cl)ccc(-c2c(Br)cnn2C)c1C#N ; CCc1c(Cl)ccc(-c2c(Br)cnn2C)c1C#N]

A mixture of 6-(4-bromo-2-methyl-pyrazol-3-yl)-3-chloro-2-methyl-benzonitrile, Intermediate D-18 (400 mg, 1.29 mmol, 1.00 eq.) in THF (5 mL) was added lithium diisopropyl amine (2.00 M, 1.29 mL, 2.00 eq.) at −78° C. and stirred at −78° C. for 30 minutes. Then methyl iodide (5.15 mmol, 0.32 mL, 4.00 eq.) was added at −78° C. and the mixture stirred for 2 hours. The reaction mixture was then quenched with ammonium chloride solution (10 mL) and extracted with dichloromethane (20 mL 3) and the combined organic extracts were washed with brine (50 mL), dried over anhydrous sodium sulfate, filtered and concentrated. The residue was purified by prep-TLC (SiO₂, petroleum ether:ethyl acetate 20%) to give 6-(4-bromo-2-methyl-pyrazol-3-yl)-3-chloro-2-ethyl-benzonitrile (280 mg, 0.86 mmol, 67% yield) as a yellow oil. LCMS [M+1]⁺=326.0; ¹H NMR (400 MHz, CDCl₃) δ=7.71 (d, J=8.4 Hz, 1H), 7.59 (s, 1H), 7.24 (d, J=8.4 Hz, 1H), 3.80 (s, 3H), 3.14-3.11 (m, 2H), 1.35-1.32 (m, 3H).

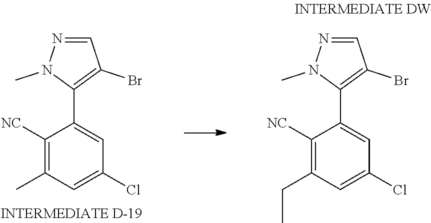 [SMILES: Cc1cc(Cl)cc(-c2c(Br)cnn2C)c1C#N ; CCc1cc(Cl)cc(-c2c(Br)cnn2C)c1C#N]

2-(4-bromo-1-methyl-1H-pyrazol-5-yl)-4-chloro-6-ethylbenzonitrile was prepared using the same method as for the preparation of Intermediate DV using Intermediate D-19 in place of Intermediate D-18 as a white solid (30 mg, 0.074 mmol, 23%). LCMS [M+1]⁺=419.2; ¹H NMR (400 MHz, DMSO-d₆) δ=12.88 (s, 1H), 8.38 (br s, 3H), 8.28 (s, 1H), 8.11 (d, J=8.4 Hz, 1H), 7.85 (d, J=2.0 Hz, 1H), 7.82 (d, J=2.0 Hz, 1H), 7.75 (d, J=1.2 Hz, 1H), 7.42 (dd, J=1.6, 8.4 Hz, 1H), 4.39-4.22 (m, 2H), 3.75 (s, 3H), 2.83 (q, J=7.6 Hz, 2H), 1.21 (t, J=7.6 Hz, 3H).

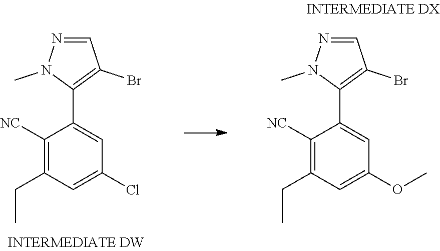

To a mixture of 2-(4-bromo-2-methyl-pyrazol-3-yl)-4-chloro-6-ethyl-benzonitrile, Intermediate DW (270 mg, 0.83 mmol, 1.00 eq.) in methanol (2 mL) was added sodium methoxide (449 mg, 8.32 mmol, 10.0 eq.) in one portion at 20° C. under nitrogen atmosphere. The mixture was stirred at 100° C. for 2 hours in a sealed tube and a light-yellow solution was formed. The mixture was then concentrated and the residue taken up in ethyl acetate (10 mL) and water (5 mL). The layers were separated, and the aqueous phase extracted with ethyl acetate (5 mL×2). The combined organic layers were washed with brine (20 mL), dried over anhydrous sodium sulfate, filtered and concentrated under reduced pressure and the residue was purified by prep-TLC (SiO₂, Petroleum ether:Ethyl acetate 25%) to give 2-(4-bromo-2-methyl-pyrazol-3-yl)-6-ethyl-4-methoxy-benzonitrile (220 mg, ˜70% purity) as a white solid. LCMS [M+1]+=321.9, ¹H NMR (400 MHz, CDCl₃) δ=7.58 (s, 1H), 6.97 (d, J=2.4 Hz, 1H), 6.77 (d, J=2.4 Hz, 1H), 3.91 (s, 3H), 3.81 (s, 3H), 2.97-2.92 (m, 2H), 1.37-1.34 (t, J=6.8 Hz 3H).

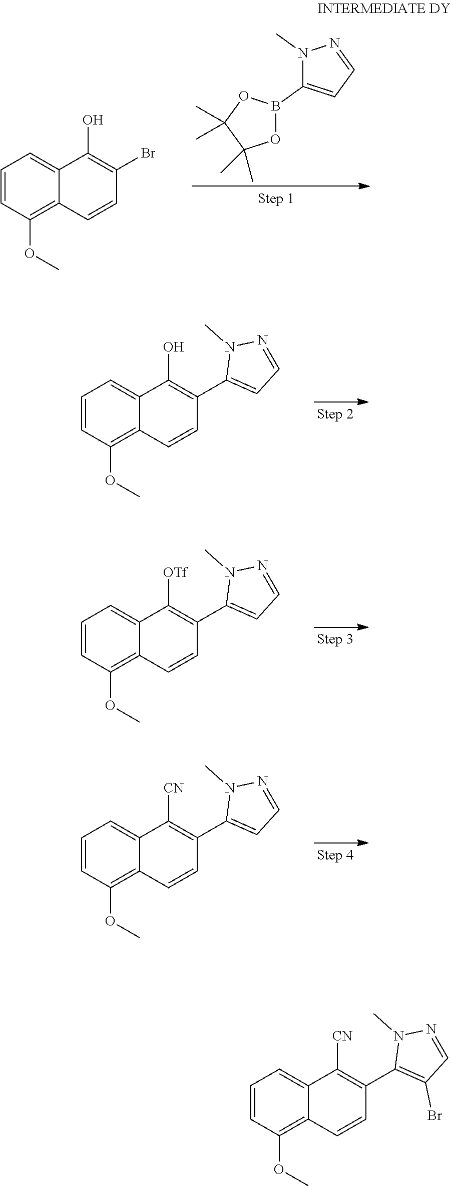 [SMILES: COc1cccc2c(O)c(Br)ccc12 ; Cn1nccc1B1OC(C)(C)C(C)(C)O1 ; COc1cccc2c(O)c(-c3ccnn3C)ccc12 ; COc1cccc2c(OS(=O)(=O)C(F)(F)F)c(-c3ccnn3C)ccc12 ; COc1cccc2c(C#N)c(-c3ccnn3C)ccc12 ; COc1cccc2c(C#N)c(-c3c(Br)cnn3C)ccc12]

Step 1: A mixture of 2-bromo-5-methoxy-naphthalen-1-ol (2.60 g, 10.3 mmol, 1.00 eq.), 1-methyl-5-(4,4,5,5-tetramethyl-1,3,2-dioxaborolan-2-yl)pyrazole (3.21 g, 15.4 mmol, 1.50 eq.), di-tert-butyl(cyclopentyl)phosphane;dichloropalladium;iron (670 mg, 1.03 mmol, 0.10 eq.) and sodium carbonate (2.18 g, 20.6 mmol, 2.00 eq.) in dioxane (30 mL) and water (6 mL) was degassed with nitrogen then stirred at 100° C. for 0.5 hour. After such time the reaction mixture was concentrated under reduced pressure and the residue was purified by column chromatography (SiO₂, petroleum ether:ethyl acetate 10-100%) to give 5-methoxy-2-(2-methylpyrazol-3-yl)naphthalen-1-ol (720 mg, 2.83 mmol, 28% yield) as a yellow solid. ¹H NMR (400 MHz, DMSO-d₆) δ=9.57 (s, 1H), 7.86 (d, J=8.6 Hz, 1H), 7.72 (d, J=8.6 Hz, 1H), 7.50 (d, J=1.6 Hz, 1H), 7.48-7.42 (m, 1H), 7.26 (d, J=8.6 Hz, 1H), 7.03 (d, J=7.6 Hz, 1H), 6.33 (d, J=1.6 Hz, 1H), 3.97 (s, 3H), 3.69 (s, 3H).

Step 2: To a solution of 5-methoxy-2-(2-methylpyrazol-3-yl)naphthalen-1-ol (650 mg, 2.56 mmol, 1.00 eq.), 4 Å molecular sieves (1.00 g) and triethylamine (7.67 mmol, 1.07 mL, 3.00 eq.) in dichloromethane (20 mL) was added Tf₂O (3.83 mmol, 0.63 mL, 1.50 eq.) in a dropwise fashion at −40° C. under nitrogen. The reaction mixture was stirred at −40° C. for 0.5 hour then concentrated under reduced pressure and the residue purified by column chromatography (SiO₂, petroleum ether:ethyl acetate 0-15%) to give [5-methoxy-2-(2-methylpyrazol-3-yl)-1-naphthyl]trifluoromethanesulfonate (341 mg, 0.79 mmol, 30% yield) as a yellow oil. LCMS [M+1]⁺=387.1; ¹H NMR (400 MHz, CDCl₃) δ=8.40 (dd, J=0.8, 8.8 Hz, 1H), 7.76 (d, J=8.8 Hz, 1H), 7.64 (d, J=8.0 Hz, 1H), 7.61 (d, J=2.0 Hz, 1H), 7.43 (d, J=8.8 Hz, 1H), 7.00 (d, J=7.6 Hz, 1H), 6.45 (d, J=2.0 Hz, 1H), 4.07 (s, 3H), 3.82 (s, 3H).

Step 3: A mixture of [5-methoxy-2-(2-methylpyrazol-3-yl)-1-naphthyl]trifluoromethanesulfonate (290 mg, 0.67 mmol, 1.00 eq.), zinc cyanide (0.81 mmol, 51.1 μL, 1.20 eq.), Pd₂(dba)₃ (612 mg, 0.067 mmol, 0.10 eq.), DPPF (74 mg, 0.134 mmol, 0.20 eq.) and zinc powder (4.4 mg, 0.067 mmol, 0.10 eq.) in DMF (10 mL) was degassed with nitrogen then stirred at 120° C. for 1 hour. The reaction mixture was then diluted with water (100 mL) and extracted with ethyl acetate (100 mL×3). The combined organic layers were washed with brine (200 mL), dried over anhydrous sodium sulfate, filtered and concentrated under reduced pressure and the residue purified by column chromatography (SiO₂, petroleum ether:ethyl acetate 10-50%) to give 5-methoxy-2-(2-methylpyrazol-3-yl)naphthalene-1-carbonitrile (156 mg, 0.59 mmol, 88% yield) as an off-white solid. LCMS [M+1]⁺=264.1; ¹H NMR (400 MHz, CDCl₃) δ=8.59 (dd, J=0.8, 8.8 Hz, 1H), 7.90 (d, J=8.4 Hz, 1H), 7.68 (t, J=8.4 Hz, 1H), 7.64 (d, J=2.0 Hz, 1H), 7.49 (d, J=8.4 Hz, 1H), 7.01 (d, J=7.6 Hz, 1H), 6.58 (d, J=2.0 Hz, 1H), 4.07 (s, 3H), 3.89 (s, 3H).

Step 4: To a solution of 5-methoxy-2-(2-methylpyrazol-3-yl)naphthalene-1-carbonitrile (180 mg, 0.68 mmol, 1.00 eq.) in acetonitrile (2 mL) was added N-bromosuccinimide (146 mg, 0.82 mmol, 1.20 eq.). The mixture was stirred at 35° C. for 0.5 hour then concentrated under reduced pressure and the residue purified by prep-TLC (SiO₂, petroleum ether:ethyl acetate 30%) to give 2-(4-bromo-2-methyl-pyrazol-3-yl)-5-methoxy-naphthalene-1-carbonitrile (174 mg, 0.51 mmol, 74% yield) as an off-white solid. LCMS [M+1]⁺=342.0; ¹H NMR (400 MHz, CDCl₃) δ=8.64 (dd, J=0.8, 8.8 Hz, 1H), 7.92 (d, J=8.4 Hz, 1H), 7.70 (t, J=8.4 Hz, 1H), 7.64 (s, 1H), 7.47 (d, J=8.8 Hz, 1H), 7.04 (d, J=7.6 Hz, 1H), 4.08 (s, 3H), 3.84 (s, 3H).

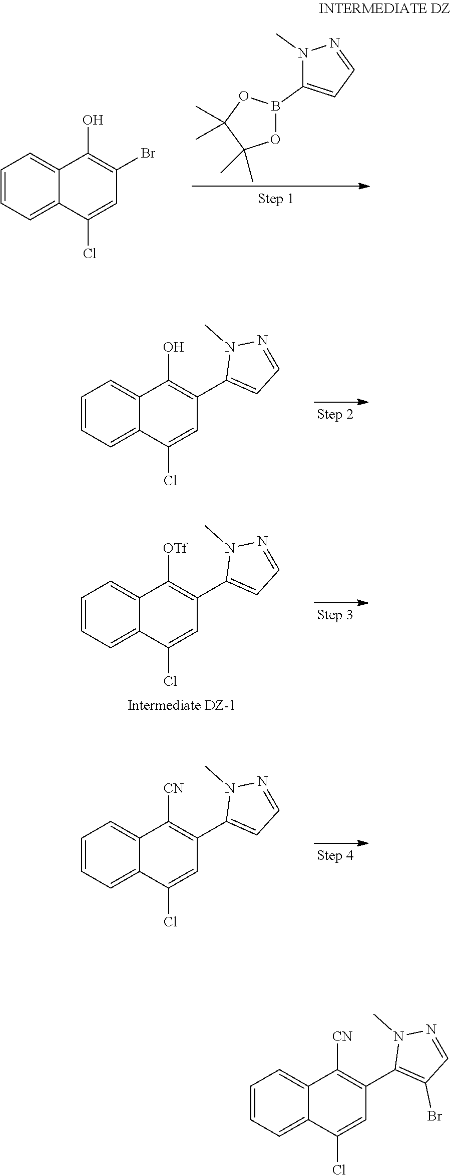 [SMILES: Oc1c(Br)cc(Cl)c2ccccc12 ; Cn1nccc1B1OC(C)(C)C(C)(C)O1 ; Cn1nccc1-c1cc(Cl)c2ccccc2c1O ; Cn1nccc1-c1cc(Cl)c2ccccc2c1OS(=O)(=O)C(F)(F)F ; Cn1nccc1-c1cc(Cl)c2ccccc2c1C#N ; Cn1ncc(Br)c1-c1cc(Cl)c2ccccc2c1C#N]

Intermediate DZ, 2-(4-bromo-1-methyl-1H-pyrazol-5-yl)-4-chloro-1-naphthonitrile was prepared as a yellow solid (25 mg, 0.072 mmol, 2% yield over 4 steps) starting from 2-bromo-4-chloro-naphthalen-1-ol according to the method described for the preparation of Intermediate DX. LCMS [M+1]⁺=347.8; ¹H NMR (400 MHz, CDCl₃d δ=8.48-8.43 (m, 1H), 8.42-8.38 (m, 1H), 7.91-7.82 (m, 2H), 7.65 (d, J=4.4 Hz, 2H), 3.88 (s, 3H).

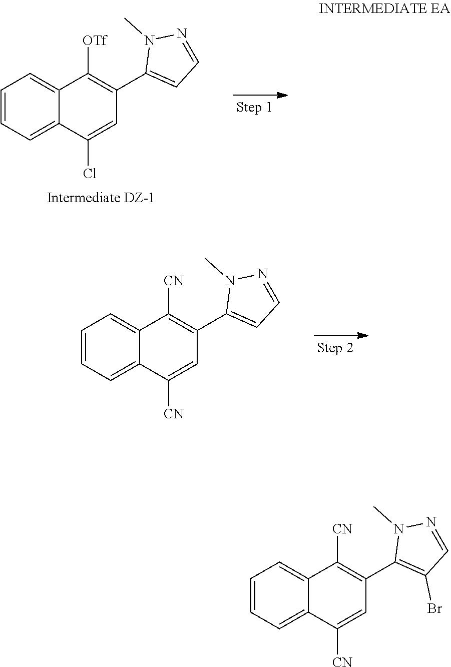 [SMILES: Cn1nccc1-c1cc(Cl)c2ccccc2c1OS(=O)(=O)C(F)(F)F ; Cn1nccc1-c1cc(C#N)c2ccccc2c1C#N ; Cn1ncc(Br)c1-c1cc(C#N)c2ccccc2c1C#N]

Step 1: A mixture of [4-chloro-2-(2-methylpyrazol-3-yl)-1-naphthyl]trifluoromethanesulfonate (38 mg, 0.097 mmol, 1.00 eq.), zinc cyanide (22 mg, 190 μmol, 12.4 μL, 2.00 eq.), DPPF (5.4 mg, 9.7 μmol, 0.10 eq.), zinc powder (640 μg, 9.7 μmol, 0.10 eq.) and Pd₂(dba)₃ (4.5 mg, 4.86 μmol, 0.05 eq.) in DMF (1.0 mL) was degassed with nitrogen then stirred at 100° C. for 4 hours. The mixture was then concentrated and the residue was purified by prep-TLC (SiO₂, petroleum ether:ethyl acetate 20%) to give 2-(2-methylpyrazol-3-yl)naphthalene-1,4-dicarbonitrile (30 mg, 93.4 μmol, 96% yield) as a yellow solid. LCMS [M+1]⁺=259.0; ¹H NMR (400 MHz, CDCl₃) δ=8.48-8.44 (m, 1H), 8.44-8.39 (m, 1H), 7.96 (s, 1H), 7.96-7.94 (m, 1H), 7.94-7.92 (m, 1H), 7.67 (d, J=2.0 Hz, 1H), 6.62 (d, J=2.0 Hz, 1H), 3.92 (s, 3H).

Step 2: A mixture of 2-(2-methylpyrazol-3-yl)naphthalene-1,4-dicarbonitrile (30 mg, 0.093 mmol, 1.00 eq.), N-bromosuccinimide (41 mg, 0.23 mmol, 2.00 eq.) in acetonitrile (2.0 mL) was degassed nitrogen then stirred at 35° C. for 2 hours. The mixture was then concentrated and the residue purified by prep-TLC (SiO₂, petroleum ether:ethyl acetate 25%) to give 2-(4-bromo-2-methyl-pyrazol-3-yl)naphthalene-1,4-dicarbonitrile (25 mg, 0.067 mmol, 58% yield) as a yellow solid. LCMS [M+1]⁺=339.0; ¹H NMR (400 MHz, CDCl₃-d) δ=8.52-8.41 (m, 2H), 8.01-7.96 (m, 2H), 7.95 (s, 1H), 7.68 (s, 1H), 3.88 (s, 3H).

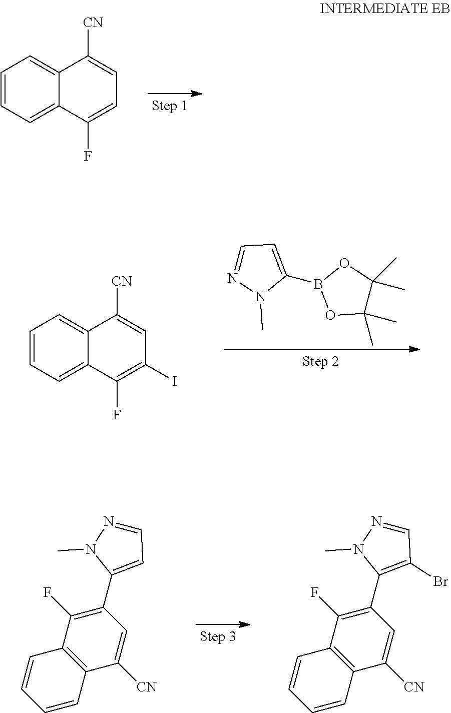 [SMILES: N#Cc1ccc(F)c2ccccc12 ; N#Cc1cc(I)c(F)c2ccccc12 ; Cn1nccc1B1OC(C)(C)C(C)(C)O1 ; Cn1nccc1-c1cc(C#N)c2ccccc2c1F ; Cn1ncc(Br)c1-c1cc(C#N)c2ccccc2c1F]

Step 1: A solution of n-butyl lithium (2.50 M, 1.87 mL, 1.00 eq.) was added dropwise over 30 min to a solution of 2,2,6,6-tetramethylpiperidine (660 mg, 4.67 mmol, 0.79 mL, 1.00 eq.) in THF (10 mL) at 0° C. and the mixture was then was cooled to −78° C. and a solution of 4-fluoronaphthalene-1-carbonitrile (0.80 g, 4.67 mmol, 1.00 eq.) in THF (3 mL) was added over 15 min. The mixture was then stirred at −78° C. for 2 hours. After such time a solution of iodine (1.19 g, 4.67 mmol, 1.00 eq.) in THF (3 mL) was added over 30 min and the reaction mixture was stirred at −78° C. for 2 hours, then allowed to warm-up to 25° C. and stirred for a further 12 hours. The reaction mixture was then quenched with water (50 mL) and extracted with ethyl acetate (30 mL×2). The combine organic layers were washed with saturated sodium thiosulfate (30 mL×3), 1 M hydrochloride (30 mL×3) and brine (50 mL) and dried over anhydrous sodium sulfate and filtered. The filtrate was concentrated, and the residue was purified by flash silica gel chromatography (ethyl acetate:petroleum ether 0-5%) to give 4-fluoro-3-iodo-naphthalene-1-carbonitrile (1.00 g, 3.37 mmol, 72% yield) as a white solid. ¹H NMR (400 MHz, CDCl₃) δ=8.15 (d, J=8.4 Hz, 1H), 8.13-8.07 (m, 2H), 7.72 (dt, J=1.2, 7.7 Hz, 1H), 7.68-7.61 (m, 1H).

Step 2: To a solution of 4-fluoro-3-iodo-naphthalene-1-carbonitrile (900 mg, 3.03 mmol, 1.00 eq.), 1-methyl-5-(4,4,5,5-tetramethyl-1,3,2-dioxaborolan-2-yl)pyrazole (1.58 g, 7.57 mmol, 2.50 eq.) and potassium phosphate (1.29 g, 6.06 mmol, 2.00 eq.) in dioxane (10 mL) and water (2 mL) was added di-tert-butyl(cyclopentyl)phosphane;dichloropalladium-iron (197 mg, 0.30 mmol, 0.10 eq.). The reaction was stirred at 80° C. for 18 hours under nitrogen atmosphere. The reaction mixture was then partitioned between water (20 mL) and ethyl acetate (10 mL), extracted with ethyl acetate (10 mL×2) and the combined the organic layers dried over anhydrous magnesium sulfate, filtered and concentrated. The residue was then purified by flash silica gel chromatography (0-25% ethyl acetate:petroleum ether gradient) to give 4-fluoro-3-(2-methylpyrazol-3-yl)naphthalene-1-carbonitrile (0.80 g, 2.87 mmol, 95% yield) as a white solid. ¹H NMR (400 MHz, CDCl₃) δ=8.33-8.25 (m, 2H), 7.91 (s, 1H), 7.88-7.82 (m, 1H), 7.82-7.75 (m, 1H), 7.63 (d, J=2.0 Hz, 1H), 6.46 (d, J=2.0 Hz, 1H), 3.89 (d, J=1.6 Hz, 3H).

Step 3: To a solution of 4-fluoro-3-(2-methylpyrazol-3-yl)naphthalene-1-carbonitrile (200 mg, 0.80 mmol, 1.00 eq.) in acetonitrile (5 mL) was added 1-bromopyrrolidine-2,5-dione (212 mg, 1.19 mmol, 1.50 eq.) and the reaction was stirred at 25° C. for 12 hours. The reaction was then concentrated and the residue purified by flash silica gel chromatography (0-15% ethyl acetate:petroleum ether) to give 3-(4-bromo-2-methyl-pyrazol-3-yl)-4-fluoro-naphthalene-1-carbonitrile (0.20 g, 0.61 mmol, 76% yield) as a gray solid. LCMS [M+1]⁺=332.1/300.1; ¹H NMR (400 MHz, CDCl₃) δ=8.32 (dd, J=8.4, 13.1 Hz, 2H), 7.95-7.86 (m, 2H), 7.84-7.76 (m, 1H), 7.64 (s, 1H), 3.85 (d, J=1.2 Hz, 3H).

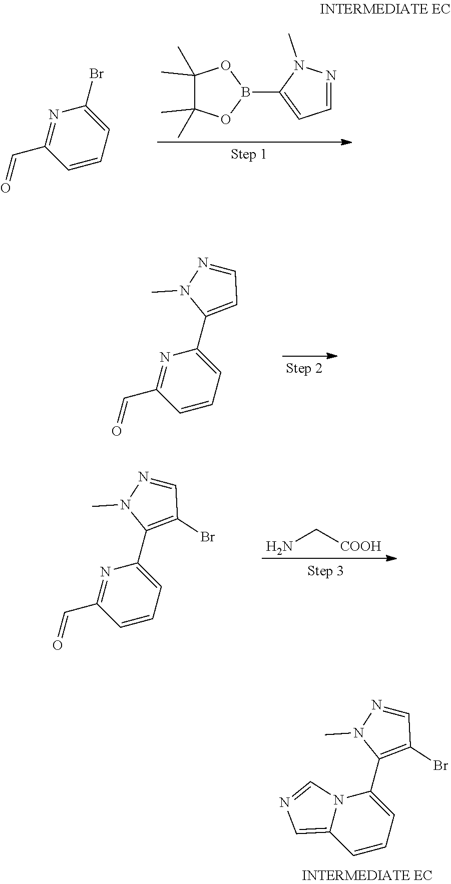 [SMILES: O=Cc1cccc(Br)n1 ; Cn1nccc1B1OC(C)(C)C(C)(C)O1 ; Cn1nccc1-c1cccc(C=O)n1 ; Cn1ncc(Br)c1-c1cccc(C=O)n1 ; NCC(=O)O ; Cn1ncc(Br)c1-c1cccc2cncn12]

Step 1: To a solution of 6-bromopicolinaldehyde (1.00 g, 5.38 mmol, 1.00 eq.) and 1-methyl-5-(4,4,5,5-tetramethyl-1,3,2-dioxaborolan-2-yl)-1H-pyrazole (1.12 g, 5.38 mmol, 1.00 eq.) in dioxane (15 mL) and water (3 mL) was added potassium carbonate (1.49 g, 10.8 mmol, 2.00 eq.) and Pd(dppf)Cl₂ (393 mg, 0.538 mmol, 0.10 eq.) at 20° C. under a nitrogen atmosphere. The mixture was then stirred at 80° C. for 6 hours. After such time the reaction mixture was diluted with water (50 mL) and extracted with dichloromethane (50 mL×2). The combined organic layers were washed with brine (15 mL×2), dried over anhydrous sodium sulfate, filtered and concentrated under reduced pressure and the residue was purified by column chromatography (SiO₂, petroleum ether:ethyl acetate 25-50%) to give 6-(1-methyl-1H-pyrazol-5-yl)picolinaldehyde (800 mg, 4.27 mmol, 80% yield) as a yellow solid. ¹H NMR (400 MHz, CDCl₃) δ=10.13 (d, J=0.8 Hz, 1H), 7.99-7.89 (m, 2H), 7.83 (dd, J=1.6, 7.2 Hz, 1H), 7.55 (d, J=2.0 Hz, 1H), 6.69 (d, J=2.0 Hz, 1H), 4.35 (s, 3H).

Step 2: To a solution of 6-(1-methyl-1H-pyrazol-5-yl)picolinaldehyde (800 mg, 4.27 mmol, 1.00 eq.) in acetonitrile (12 mL) was added N-bromo-succinimide (1.14 g, 6.41 mmol, 1.50 eq.) at 20° C. and the mixture was stirred for 16 hours. After such time the mixture was concentrated and the residue was purified by prep-TLC (SiO₂, petroleum ether:ethyl acetate 25%) to give 6-(4-bromo-1-methyl-1H-pyrazol-5-yl)picolinaldehyde (750 mg, 2.82 mmol, 66% yield) as a white solid. ¹H NMR (400 MHz, CDCl₃) δ=10.13 (s, 1H), 8.08-7.98 (m, 3H), 7.58 (s, 1H), 4.15 (s, 3H).

Step 3: To a solution of 6-(4-bromo-1-methyl-1H-pyrazol-5-yl)picolinaldehyde (250 mg, 0.94 mmol, 1.00 eq.) in DMF (3 mL) was added 2-aminoacetic acid (78 mg, 1.03 mmol, 1.10 eq.), iodine (238 mg, 0.940 mmol, 0.19 mL, 1.00 eq), sodium bicarbonate (158 mg, 1.88 mmol, 2.00 eq.) at 20° C. The mixture was then stirred at 60° C. for 6 hours then diluted with water (20 mL) and extracted with ethyl acetate (20 mL×2). The combined organic layers were washed with brine (10 mL×2), dried over anhydrous sodium sulfate, filtered, concentrated and the residue purified by prep-TLC (SiO₂, ethyl acetate) to give 5-(4-bromo-1-methyl-1H-pyrazol-5-yl)imidazo[1,5-a]pyridine (35 mg, 0.126 mmol, 13% yield) as a yellow solid. ¹H NMR (400 MHz, CDCl₃) δ=7.77 (s, 1H), 7.68 (s, 1H), 7.64-7.59 (m, 2H), 6.87 (dd, J=6.4, 9.2 Hz, 1H), 6.66 (d, J=6.4 Hz, 1H), 3.79 (s, 3H).

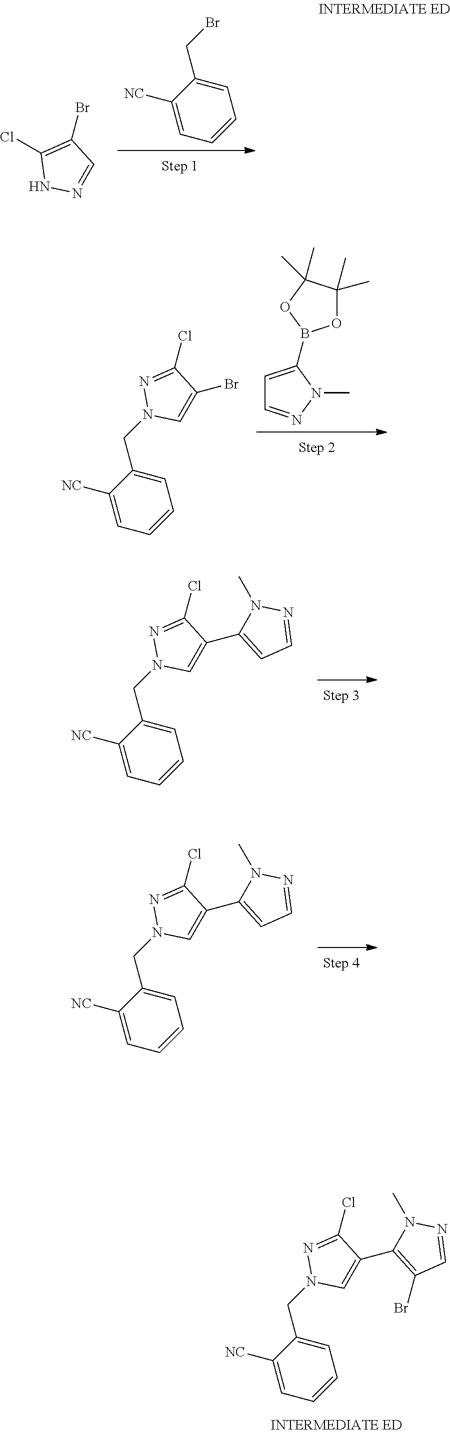

Step 1: To a solution of 4-bromo-5-chloro-1H-pyrazole (1.00 g, 5.51 mmol, 1.00 eq.) 2-(bromomethyl)benzonitrile (1.08 g, 5.51 mmol, 1.00 eq.) in acetonitrile (20 mL) was added potassium carbonate (914 mg, 6.61 mmol, 1.20 eq.) and the mixture was stirred at 80° C. for 10 hours under a nitrogen atmosphere. After such time the reaction was quenched by water (200 mL) and then extracted with ethyl acetate (150 mL×3). The combined organic extracts were washed with brine (200. mL), dried over anhydrous sodium sulfate, filtered, concentrated and the formed residue was purified by column chromatography (SiO₂, petroleum ether:ethyl acetate=10:1) to give 2-((4-bromo-3-chloro-1H-pyrazol-1-yl)methyl)benzonitrile (1.00 g, 3.37 mmol, 61% yield) as a white solid. LCMS [M+1]⁺=297.9, ¹H NMR (400 MHz, DMSO-d₆) δ=8.27 (s, 1H), 7.89 (dd, J=0.8, 7.6 Hz, 1H), 7.77-7.68 (m, 1H), 7.61-7.49 (m, 1H), 7.37 (d, J=7.6 Hz, 1H), 5.52 (s, 2H).

Step 2: To a solution of 2-((4-bromo-3-chloro-1H-pyrazol-1-yl)methyl)benzonitrile (400 mg, 1.35 mmol, 1.00 eq.), 1-methyl-5-(4,4,5,5-tetramethyl-1,3,2-dioxaborolan-2-yl)-1H-pyrazole (561 mg, 2.70 mmol, 2.00 eq.) and sodium bicarbonate (227 mg, 2.70 mmol, 2.00 eq.) in dioxane (10 mL) and water (1 mL) was added Pd(dppf)Cl₂ (99 mg, 0.135 mmol, 0.10 eq.) under a nitrogen atmosphere. The mixture was stirred at 110° C. for 10 hours and then the reaction was quenched by adding water (200 mL) then extracted with ethyl acetate (150 mL×3). The combined organic phases were washed with brine (200 mL), dried over anhydrous sodium sulfate, filtered, concentrated and the residue was purified by column chromatography (SiO₂, petroleum ether:ethyl acetate 30%) to give 2-((3′-chloro-2-methyl-1′H,2H-[3,4′-bipyrazol]-1′-yl)methyl)benzonitrile (200 mg, 0.672 mmol, 50% yield) as a white solid. LCMS [M+1]=298.0; ¹H NMR (400 MHz, DMSO-d₆) δ=8.41 (s, 1H), 7.91 (d, J=7.6 Hz, 1H), 7.79-7.70 (m, 1H), 7.62-7.52 (m, 1H), 7.50-7.42 (m, 2H), 6.42 (d, J=2.0 Hz, 1H), 5.58 (s, 2H), 3.81 (s, 3H).

Step 3: To a solution of 2-((3′-chloro-2-methyl-1′H,2H-[3,4′-bipyrazol]-1′-yl)methyl)benzonitrile (200 mg, 0.672 mmol, 1.00 eq.) in acetonitrile (10 mL) was added N-bromosuccinimide (132 mg, 0.739 mmol, 1.10 eq.) and the mixture was stirred at 25° C. for 10 hours under nitrogen atmosphere. After such time the reaction was quenched with water (50 mL) and extracted with ethyl acetate (40 mL×3). The combined organic phases was washed with brine (50 mL), dried over anhydrous sodium sulfate, filtered, concentrated and the residue was purified by column chromatography (SiO₂, petroleum ether:ethyl acetate 5%) to give 2-((4-bromo-3′-chloro-2-methyl-1′H,2H-[3,4′-bipyrazol]-1′-yl)methyl)benzonitrile (130 mg, 0.345 mmol, 51% yield) as a yellow solid. LCMS [M+1]⁺=377.9; ¹H NMR (400 MHz, DMSO-d₆) δ=8.37 (s, 1H), 7.92 (d, J=7.6 Hz, 1H), 7.83-7.71 (m, 1H), 7.67 (s, 1H), 7.62-7.55 (m, 1H), 7.45 (d, J=8.0 Hz, 1H), 5.61 (s, 2H), 3.73 (s, 3H).

Step 4: To a solution of tert-butyl ((4-oxo-7-(4,4,5-trimethyl-1,3,2-dioxaborolan-2-yl)-3,4-dihydrophthalazin-1-yl)methyl)carbamate (256 mg, 0.637 mmol, 2.00 eq.), 2-((4-bromo-3′-chloro-2-methyl-1′H,2H-[3,4′-bipyrazol]-1′-yl)methyl)benzonitrile (120 mg, 0.319 mmol, 1.00 eq.), sodium bicarbonate (54 mg, 0.637 mmol, 25 μL, 2.00 eq.) in water (0.5 mL) and dioxane (5 mL) was added Pd(dtbpf)Cl₂ (21 mg, 32 μmol, 0.10 eq.) under a nitrogen atmosphere and then the mixture was stirred at 110° C. for 10 hours. The reaction was then quenched with water (50 mL), extracted with ethyl acetate (40 mL×3) and the combined organic phases were washed with brine (50 mL), dried over anhydrous sodium sulfate, filtered and concentrated in vacuo. The residue was purified by column chromatography (SiO₂, petroleum ether:ethyl acetate 15%) to give tert-butyl((7-(3′-chloro-1′-(2-cyanobenzyl)-2-methyl-1′H,2H-[3,4′-bipyrazol]-4-yl)-4-oxo-3,4-dihydrophthalazin-1-yl)methyl)carbamate (115 mg, 0.201 mmol, 63% yield) as a black brown oil. LCMS [M+1]⁺=571.1; ¹H NMR (400 MHz, DMSO-d₆) δ=12.48 (s, 1H), 11.94 (s, 1H), 8.42 (s, 1H), 8.13-8.05 (m, 2H), 7.92 (d, J=7.6 Hz, 1H), 7.80-7.72 (m, 1H), 7.62-7.55 (m, 2H), 7.47 (d, J=8.0 Hz, 1H), 7.40-7.31 (m, 1H), 5.64 (s, 2H), 4.36 (br d, J=5.6 Hz, 2H), 3.74 (s, 3H), 1.38 (s, 9H).

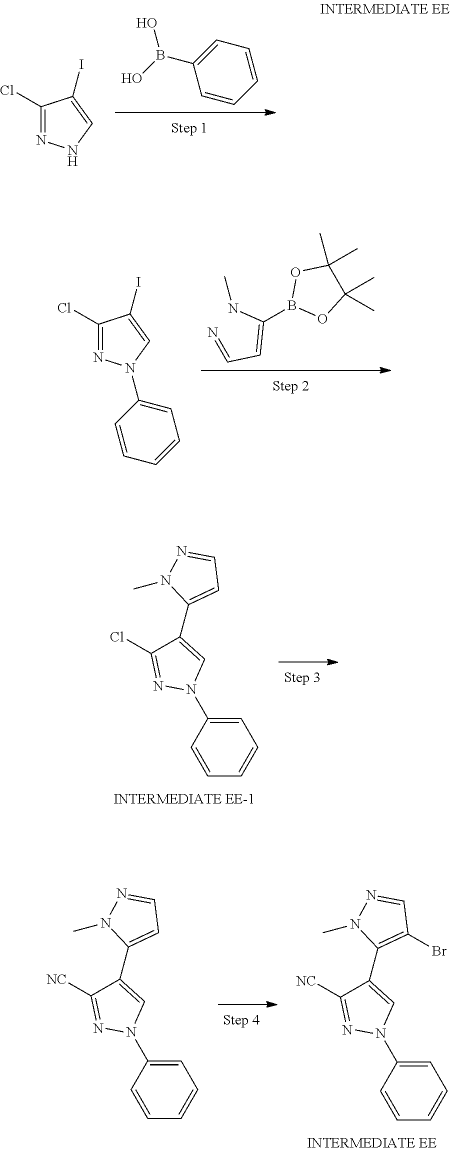

Step 1: To a mixture of phenylboronic acid (1.92 g, 15.8 mmol, 2.00 eq.) and 3-chloro-4-iodo-1H-pyrazole (1.80 g, 7.88 mmol, 1.00 eq.) in dichloromethane (30 mL) was added pyridine (1.86 g, 23.5 mmol, 1.90 mL, 2.99 eq.) and copper acetate (1.72 g, 9.46 mmol, 1.20 eq.) in one portion. The mixture was stirred at 20° C. for 16 hours, then filtered and concentrated and the residue purified by flash silica gel chromatography (0-5% ethyl acetate:petroleum ether) to give 3-chloro-4-iodo-1-phenyl-pyrazole (1.50 g, 4.93 mmol, 63% yield) as a yellow liquid. LCMS [M+1]+=305.0; ¹H NMR (400 MHz, CDCl₃) δ 7.91 (s, 1H), 7.63-7.59 (m, 2H), 7.49-7.43 (m, 2H), 7.36-7.30 (m, 1H).

Step 2: 1-methyl-5-(4,4,5,5-tetramethyl-1,3,2-dioxaborolan-2-yl)pyrazole (1.23 g, 5.91 mmol, 1.50 eq.), 3-chloro-4-iodo-1-phenyl-pyrazole (1.20 g, 3.94 mmol, 1.00 eq.), potassium phosphate (1.67 g, 7.88 mmol, 2.00 eq.) and di-tert-butyl(cyclopentyl)phosphane;dichloropalladium-iron (256 mg, 0.39 mmol, 0.10 eq.) in dioxane (20 mL) and water (4 mL) was de-gassed and then heated to 80° C. for 16 hours under a nitrogen atmosphere. The reaction mixture was then concentrated, and the residue diluted with water (10 mL) and extracted with ethyl acetate (10 mL 3), dried over sodium sulfate, concentrated under reduced pressure and the residue purified by column chromatography (SiO₂, petroleum ether:ethyl acetate 10-15%) to give 3-chloro-4-(2-methylpyrazol-3-yl)-1-phenyl-pyrazole, Intermediate EE-1 (0.80 g, 3.09 mmol, 79% yield) as a yellow oil. LCMS [M+1]⁺=258.9: ¹H NMR (400 MHz, CDCl₃) δ 7.97 (s, 1H), 7.71-7.67 (m, 2H), 7.55 (d, J=2.0 Hz, 1H), 7.49 (t, J=8.0 Hz, 2H), 7.39-7.34 (m, 1H), 6.43 (d, J=2.0 Hz, 1H), 3.91 (s, 3H).

Step 3: A mixture of 3-chloro-4-(2-methylpyrazol-3-yl)-1-phenyl-pyrazole (210 mg, 0.811 mmol, 1.00 eq.), tetrapotassium-hexacyanoiron(4-) trihydrate (1.03 g, 2.44 mmol, 3.00 eq.) and [2-(2-aminophenyl)phenyl]-methylsulfonyloxy-palladium-dicyclohexyl-[3,6-dimethoxy-2-(2,4,6-triisopropylphenyl)phenyl]phosphane (73.6 mg, 0.081 mmol, 0.10 eq.) in dimethylacetamide (6 mL) and water (3 mL) was heated to 100° C. for 16 hours under nitrogen atmosphere. The reaction mixture was then diluted with water (10 mL) and extracted with ethyl acetate (10 mL×3). The combined organic layers were washed with brine (15 mL), dried over sodium sulfate, filtered, concentrated under reduced pressure and the residue purified by column chromatography (SiO₂, petroleum ether:ethyl acetate 10-15%) to give 4-(2-methylpyrazol-3-yl)-1-phenyl-pyrazole-3-carbonitrile (200 mg, 0.80 mmol, 99% yield) as a yellow solid. LCMS [M+1]⁺=249.9; ¹H NMR (400 MHz, CDCl₃) δ=8.09 (s, 1H), 7.76-7.72 (m, 2H), 7.56 (d, J=2.0 Hz, 1H), 7.51-7.56 (m, 2H), 7.43-7.48 (m, 1H), 6.59 (d, J=2.0 Hz, 1H), 3.98 (s, 3H).

Step 4: To a mixture of 4-(2-methylpyrazol-3-yl)-1-phenyl-pyrazole-3-carbonitrile (180 mg, 0.722 mmol, 1.00 eq.) in acetonitrile (5 mL) was added N-bromosuccinimide (192 mg, 1.08 mmol, 1.50 eq.). The mixture was stirred at 20° C. for 16 hours. The reaction mixture was then quenched with saturated sodium sulfite (15 mL), extracted with ethyl acetate (15 mL×3), dried over anhydrous sodium sulfate, concentrated and the residue purified by column chromatography (SiO₂, petroleum ether:ethyl acetate 10-20%) to give 4-(4-bromo-2-methyl-pyrazol-3-yl)-1-phenyl-pyrazole-3-carbonitrile (220 mg, 0.67 mmol, 93% yield) as a yellow solid. LCMS [M+1]⁺=327.9; ¹H NMR (500 MHz, CDCl₃)=8.17 (s, 1H), 7.78-7.74 (m, 2H), 7.59 (s, 1H), 7.58-7.54 (m, 2H), 7.49-7.45 (m, 1H), 3.95 (s, 3H).

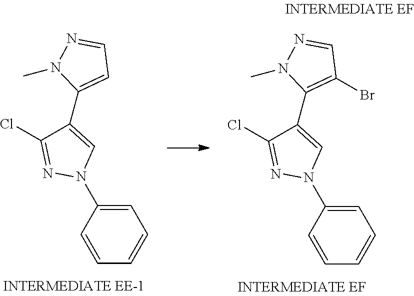 [SMILES: Cn1nccc1-c1cn(-c2ccccc2)nc1Cl ; Cn1ncc(Br)c1-c1cn(-c2ccccc2)nc1Cl]

To a mixture of 3-chloro-4-(2-methylpyrazol-3-yl)-1-phenyl-pyrazole, Intermediate EE-1 (200 mg, 0.773 mmol, 1.00 eq.) in acetonitrile (1 mL) was added 1-bromopyrrolidine-2,5-dione (165 mg, 0.927 mmol, 1.20 eq.) in one portion at 20° C. The mixture was stirred at 20° C. for 16 hours. The reaction mixture was then concentrated and the residue purified by flash silica gel chromatography (0-17% ethyl acetate:petroleum ether) to give 4-bromo-5-(3-chloro-1-phenyl-pyrazol-4-yl)-1-methyl-pyrazole (200 mg, 0.592 mmol, 77% yield) as a yellow oil. ¹H NMR (500 MHz, CDCl₃) δ=8.07-8.04 (m, 1H), 7.72 (dd, J=1.2, 8.4 Hz, 2H), 7.58 (s, 1H), 7.54-7.49 (m, 2H), 7.42-7.35 (m, 1H), 3.88 (s, 3H).

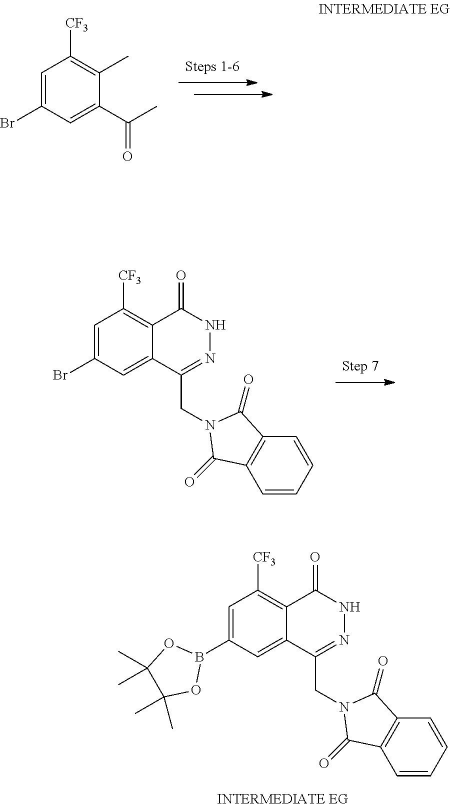 [SMILES: CC(=O)c1cc(Br)cc(C(F)(F)F)c1C ; O=C1c2ccccc2C(=O)N1Cc1n[nH]c(=O)c2c(C(F)(F)F)cc(Br)cc12 ; CC1(C)OB(c2cc(C(F)(F)F)c3c(=O)[nH]nc(CN4C(=O)c5ccccc5C4=O)c3c2)OC1(C)C]

Steps 1-6: 2-((7-bromo-4-oxo-5-(trifluoromethyl)-3,4-dihydrophthalazin-1-yl)methyl)isoindoline-1,3-dione was prepared as a white solid (0.50 g, 1.11 mmol, 6% yield over 6 steps) starting from 1-(5-bromo-2-methyl-3-(trifluoromethyl)phenyl)ethan-1-one following the same procedure described for the first 6 steps of Intermediate DK. LCMS [M+1]⁺=454.0; ¹H NMR (400 MHz, DMSO-d₆) δ=12.75 (s, 1H), 8.74 (d, J=1.6 Hz, 1H), 8.41 (s, 1H), 7.97-7.92 (m, 2H), 7.91-7.86 (m, 2H), 5.22 (s, 2H).

Step 7: A mixture of 2-((7-bromo-4-oxo-5-(trifluoromethyl)-3,4-dihydrophthalazin-1-yl)methyl)isoindoline-1,3-dione (50 mg, 0.111 mmol, 1.00 eq.), bis(pinacolato)diboron (34 mg, 0.133 mmol, 1.20 eq.), Pd(dppf)Cl₂ (8 mg, 0.011 mmol, 0.10 eq.) and potassium acetate (22 mg, 0.221 mmol, 2.00 eq.) in dioxane (2 mL) was degassed with nitrogen and stirred at 100° C. for 1 hour. The reaction mixture was then concentrated under reduced pressure to give [4-[(1,3-dioxoisoindolin-2-yl)methyl]-1-oxo-8-(trifluoromethyl)-2H-phthalazin-6-yl]boronic acid (46.0 mg, crude) as a brown solid. LCMS [M−81]⁺=418.1.

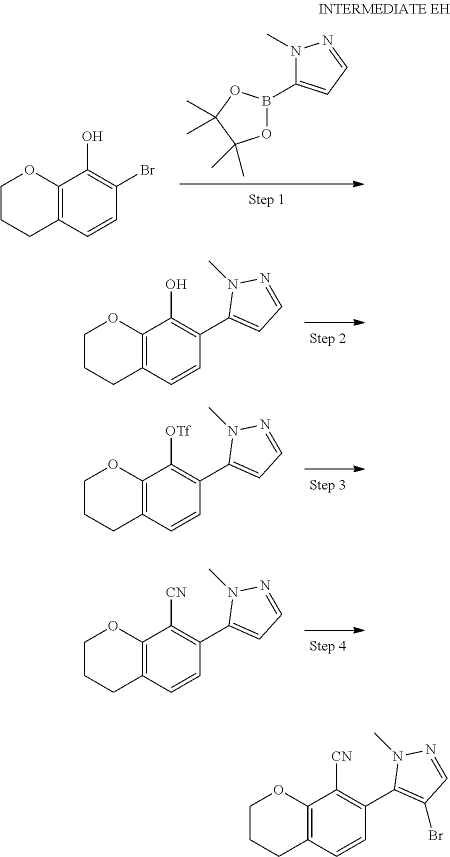

7-(4-bromo-1-methyl-1H-pyrazol-5-yl)chromane-8-carbonitrile, Intermediate EH was prepared as a white solid (21 mg, 0.049 mmol, 84% yield) using the same 4 step procedure as used for the preparation of Intermediate DY but starting with 7-bromochroman-8-ol. LCMS [M+1]⁺=413.2; ¹H NMR (400 MHz, DMSO-d₄) δ=12.40 (br s, 1H), 8.17 (s, 1H), 8.12 (d, J=8.4 Hz, 1H), 7.70 (dd, J=1.6, 8.0 Hz, 1H), 7.60 (d, J=8.0 Hz, 1H), 7.58 (d, J=1.6 Hz, 1H), 7.14 (d, J=7.6 Hz, 1H), 4.35 (br t, J=4.8 Hz, 2H), 3.72 (s, 3H), 3.68 (d, J=2.0 Hz, 2H), 2.89 (br t, J=6.0 Hz, 2H), 2.05-2.00 (m, 2H).

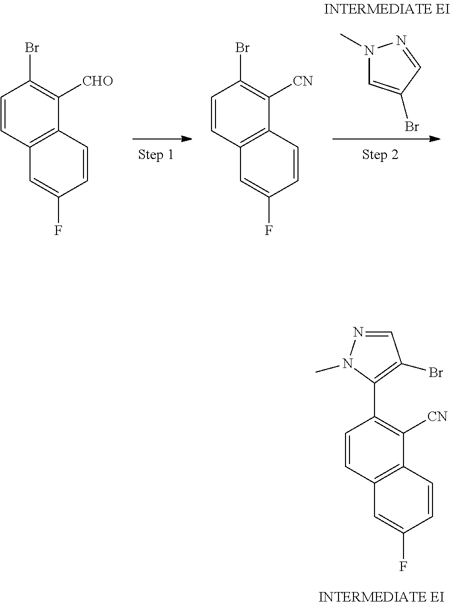

2-(4-bromo-1-methyl-1H-pyrazol-5-yl)-6-fluoro-1-naphthonitrile, Intermediate EI was prepared as a white solid (60 mg, 0.182 mmol, 31% yield over 2 steps) following the same procedure as described for the preparation of Intermediate DC starting from 2-bromo-6-fluoro-1-naphthaldehyde. LCMS [M+1]⁺=329.8/331.8; ¹H NMR (400 MHz, CCDCl₃-d) δ=8.39 (dd, J=5.2, 9.2 Hz, 1H), 8.15 (d, J=8.6 Hz, 1H), 7.68-7.63 (m, 2H), 7.62-7.56 (m, 1H), 7.54 (d, J=8.6 Hz, 1H), 3.86 (s, 3H).

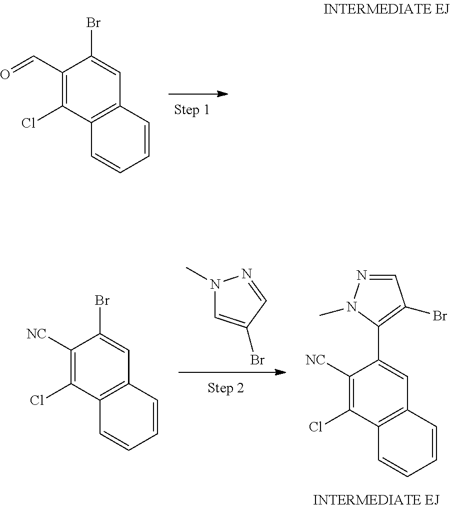

3-(4-bromo-1-methyl-1H-pyrazol-5-yl)-1-chloro-2-naphthonitrile, Intermediate EJ was prepared as a light yellow solid (35 mg, 0.101 mmol, 18% yield over 2 steps) following the same procedure as described for the preparation of Intermediate DC starting from 3-bromo-1-chloro-2-naphthaldehyde. ¹H NMR (400 MHz, CCDCl₃-d) δ=10.60 (s, 1H), 8.47-8.41 (m, 1H), 8.11 (s, 1H), 7.89-7.77 (m, 1H), 7.74-7.66 (m, 2H).

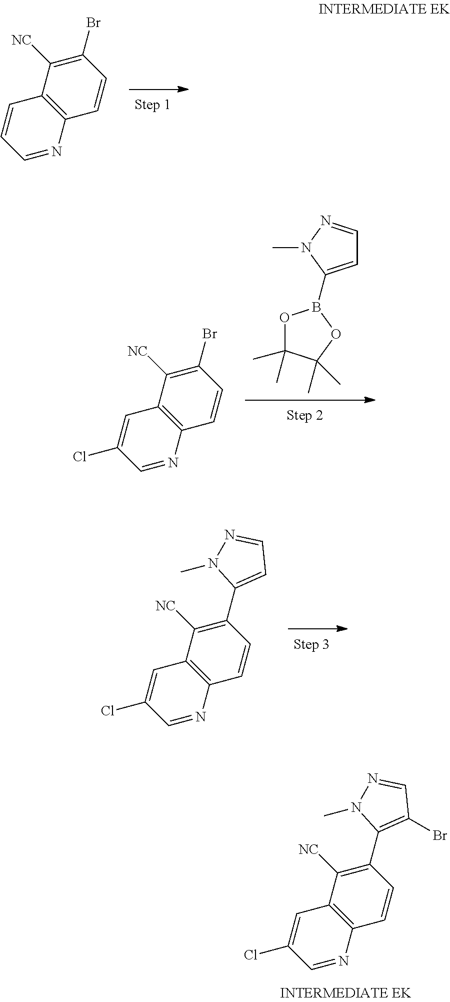

Step 1: To a solution of 6-bromoquinoline-5-carbonitrile (1.00 g, 4.29 mmol, 1.00 eq.) in acetic acid (20 mL) was added N-chlorosuccinimide (5.73 g, 42.9 mmol, 10.0 eq.). The mixture was stirred at 135° C. for 24 hours. The pH of the reaction mixture was then adjusted to pH 7 with 2 N sodium hydroxide aqueous solution (5 mL), diluted with water (50 mL) and extracted with dichloromethane (30 mL×3). The combined organic layers were washed with brine (100 mL), dried over anhydrous sodium sulfate, filtered, concentrated and the residue was purified by column chromatography (SiO₂, petroleum ether:ethyl acetate 10-15%) to give 6-bromo-3-chloro-quinoline-5-carbonitrile (512 mg, 1.91 mmol, 45% yield) as an off-white solid. LCMS [M+1]⁺=269.0; ¹H NMR (400 MHz, CDCl₃) δ=8.94 (s, 1H), 8.49 (dd, J=0.8, 2.4 Hz, 1H), 8.18 (s, 1H), 7.93 (d, J=9.2 Hz, 1H).

Step 2: A mixture of 6-bromo-3-chloro-quinoline-5-carbonitrile (512 mg, 1.91 mmol, 1.00 eq.), 1-methyl-5-(4,4,5,5-tetramethyl-1,3,2-dioxaborolan-2-yl)pyrazole (398 mg, 1.91 mmol, 1.00 eq.), di-tert-butyl(cyclopentyl)phosphane;dichloropalladium-iron (125 mg, 0.19 mmol, 0.10 eq.), sodium bicarbonate (322 mg, 3.83 mmol, 0.15 mL, 2.00 eq.) in dioxane (10 mL) and water (2 mL) was degassed with nitrogen and stirred at 80° C. for 0.5 hour. After such time the reaction mixture was concentrated under reduced pressure and the residue was purified by prep-TLC (SiO₂, petroleum ether:ethyl acetate 30%) to give 3-chloro-6-(2-methylpyrazol-3-yl)quinoline-5-carbonitrile (250 mg, 0.930 mmol, 49/a yield) as a yellow solid. LCMS [M+1]⁺=269.1; ¹H NMR (400 MHz, CDCl₃) δ=9.00 (d, J=2.4 Hz, 1H), 8.61 (d, J=2.0 Hz, 1H), 8.42 (d, J=8.8 Hz, 1H), 7.78 (d, J=8.8 Hz, 1H), 7.66 (d, J=1.6 Hz, 1H), 6.63 (d, J=1.6 Hz, 1H), 3.92 (s, 3H).

Step 3: To a solution of 3-chloro-6-(2-methylpyrazol-3-yl)quinoline-5-carbonitrile (249 mg, 0.927 mmol, 1.00 eq.) in acetonitrile (5 mL) was added N-bromosuccinimide (214 mg, 1.20 mmol, 1.30 eq.). The mixture was stirred at 35° C. for 0.5 hour. The reaction mixture was concentrated under reduced pressure and the residue was purified by column chromatography (SiO₂, petroleum ether:ethyl acetate 10-15%) to give 6-(4-bromo-2-methyl-pyrazol-3-yl)-3-chloro-quinoline-5-carbonitrile (289 mg, 0.831 mmol, 90% yield) as a yellow solid. LCMS [M+1]=349.0; ¹H NMR (400 MHz, CDCl₃) δ=9.04 (d, J=2.4 Hz, 1H), 8.64 (dd, J=0.8, 2.4 Hz, 1H), 8.49-8.46 (m, 1H), 7.76 (d, J=8.8 Hz, 1H), 7.67 (s, 1H), 3.87 (s, 3H).

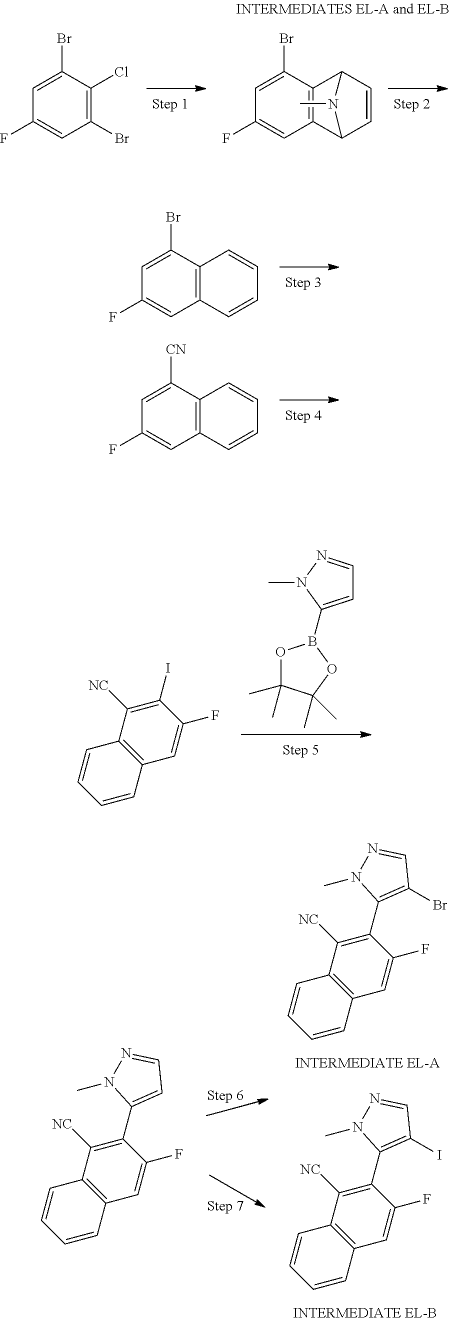

Step 1: To a solution of 1,3-dibromo-2-chloro-5-fluoro-benzene (61.0 g, 212 mmol, 1.00 eq.) and 1-methylpyrrole (34.3 g, 423 mmol, 37.7 mL, 2.00 eq.) in toluene (1500 mL) was added n-butyl lithium (2.50 M in THF, 88.9 mL, 1.05 eq.) dropwise at −30° C. under nitrogen. The mixture was then stirred at −30° C. for 0.5 hour then allowed to warm to 25° C. and stirred for 12 hours. After such time the reaction mixture was quenched with water (20 mL) and concentrated under reduced pressure and the residue was diluted with ethyl acetate (1000 mL), washed with brine (1000 mL), dried over anhydrous sodium sulfate, filtered, concentrated and the residue was purified by column chromatography (SiO₂, petroleum ether:ethyl acetate 10-50/o) to give 3-bromo-5-fluoro-11-methyl-11-azatricyclo[6.2.1.02,7]undeca-2 (7),3,5,9-tetraene (30.0 g, 118 mmol, 56% yield) as a brown liquid. ¹H NMR (400 MHz, CDCl₃) δ=7.10-6.65 (m, 4H), 4.83-4.44 (m, 2H), 2.31-2.09 (m, 3H).

Step 2: To a solution of 3-bromo-5-fluoro-11-methyl-11-azatricyclo[6.2.1.02,7]undeca-2 (7),3,5,9-tetraene (61.5 g, 242 mmol, 1.00 eq.) in chloroform (1300 mL) was carefully added m-CPBA (98.2 g, 484 mmol, 85% purity, 2.00 eq.) in portions maintaining the inner temperature below 40° C. After 2 hours, the brown solution turned yellow and the mixture was stirred at 25° C. for a further 24 hours. After such time the mixture was diluted with dichloromethane (1000 mL) and washed with saturated sodium sulfite (1500 mL×2) followed by brine (1500 mL), then dried over anhydrous sodium sulfate, filtered, concentrated and the residue was purified by column chromatography (SiO₂, petroleum ether:ethyl acetate 0-10%) to give 1-bromo-3-fluoro-naphthalene (37.8 g, 168 mmol, 70% yield) as a colorless liquid. ¹H NMR (400 MHz, CDCl₃) δ=8.25-8.18 (m, 1H), 7.75 (br d, J=3.2 Hz, 1H), 7.63 (dd, J=2.4, 8.0 Hz, 1H), 7.58-7.52 (m, 2H), 7.47-7.40 (m, 1H).

Step 3: A mixture of 1-bromo-3-fluoro-naphthalene (34.8 g, 155 mmol, 1.00 eq.), Pd₂(dba)₃ (14.2 g, 15.5 mmol, 0.10 eq.), zinc cyanide (45.4 g, 387 mmol, 24.5 mL, 2.50 eq.), DPPF (17.1 g, 30.9 mmol, 0.20 eq.) and Zn power (1.01 g, 15.5 mmol, 0.10 eq.) in DMF (400 mL) was degassed with nitrogen and then the mixture was stirred at 115° C. for 4 hours. After such time the mixture was filtered, diluted with ethyl acetate (1000 mL), washed with brine (1000 mL×2), dried over anhydrous sodium sulfate, filtered, concentrated and the residue was purified by column chromatography (SiO₂, petroleum ether:ethyl acetate 0-10%) to give 3-fluoronaphthalene-1-carbonitrile (21.5 g, 126 mmol, 81% yield) as a yellow solid. ¹H NMR (400 MHz, CDCl₃) δ=8.26-8.21 (m, 1H), 7.92-7.85 (m, 1H), 7.76-7.65 (m, 4H).

Step 4: n-butyl lithium (2.50 M in hexane, 2.83 mL, 1.10 eq.) was added to a solution of N-isopropylpropan-2-amine (845 mg, 8.35 mmol, 1.18 mL, 1.30 eq.) in THF (15 mL) at −70° C. and the reaction mixture was stirred at −70° C. for 15 minutes and then 3-fluoronaphthalene-1-carbonitrile (1.10 g, 6.43 mmol, 1.00 eq.) in THF (2 mL) was added to the mixture and the reaction mixture was stirred for 30 minutes at −70° C. A solution of iodine (2.12 g, 8.35 mmol, 1.30 eq.) in THF (2.0 mL) was then added to the reaction mixture at −70° C. and the solution was stirred at −70° C. for a further 30 minutes and then the mixture was warmed to 25° C. and stirred at 25° C. for 10 hours. After such time the reaction was quenched with water (100 mL), and diluted with ethyl acetate (250 mL), washed with saturated sodium thiosulfate (100 mL×2) and brine (250 mL). The organic phase was dried over anhydrous sodium sulfate filtered, concentrated and the formed residue was purified by column chromatography (SiO₂, petroleum ether:ethyl acetate 0-15%) to give 3-fluoro-2-iodo-naphthalene-1-carbonitrile (1.70 g, 5.72 mmol, 89% yield) as a white solid. ¹H NMR (400 MHz, CDCl₃) δ=8.36-8.24 (m, 1H), 7.83-7.71 (m, 1H), 7.59-7.48 (m, 3H).

Step 5: A mixture of 3-fluoro-2-iodo-naphthalene-1-carbonitrile (800 mg, 2.69 mmol, 1.00 eq.), 1-methyl-5-(4,4,5,5-tetramethyl-1,3,2-dioxaborolan-2-yl)pyrazole (1.23 g, 5.92 mmol, 2.20 eq.), Pd(dtbpf)Cl₂ (176 mg, 0.269 mmol, 0.10 eq.), sodium bicarbonate (679 mg, 8.08 mmol, 3.00 eq.) in dioxane (10 mL) and water (2 mL) was degassed with nitrogen and stirred at 80° C. for 12 hours. After such time the mixture was concentrated and the residue purified by column chromatography (SiO₂, petroleum ether:ethyl acetate 5-50%) to give 3-fluoro-2-(2-methylpyrazol-3-yl)naphthalene-1-carbonitrile (500 mg, 1.99 mmol, 74% yield) as a yellow solid. LCMS [M+1]⁺=252.1; ¹H NMR (400 MHz, CDCl₃) δ=8.35-8.27 (m, 1H), 8.00-7.92 (m, 1H), 7.87 (d, J=9.6 Hz, 1H), 7.79-7.70 (m, 2H), 7.68 (d, J=2.0 Hz, 1H), 6.62 (d, J=2.0 Hz, 1H), 3.85 (d, J=1.2 Hz, 3H).

Step 6: To a solution of 3-fluoro-2-(2-methylpyrazol-3-yl)naphthalene-1-carbonitrile (500 mg, 1.99 mmol, 1.00 eq.) in acetonitrile (8 mL) was added N-bromosuccinimide (638 mg, 3.58 mmol, 1.80 eq.). The mixture was stirred at 25° C. for 3 hours. After such time the mixture was concentrated and the residue was purified by column chromatography (SiO₂, petroleum ether:ethyl acetate 10-50%) to give 2-(4-bromo-2-methyl-pyrazol-3-yl)-3-fluoro-naphthalene-1-carbonitrile (550 mg, 1.56 mmol, 78% yield) as a yellow solid. LCMS [M+1]⁺=331.9; ¹H NMR (400 MHz, CDCl₃) δ=8.40-8.31 (m, 1H), 8.01-7.95 (m, 1H), 7.91 (d, J=9.6 Hz, 1H), 7.79-7.73 (m, 2H), 7.68 (s, 1H), 3.84 (s, 3H).

Step 7: To a solution of 3-fluoro-2-(2-methylpyrazol-3-yl)naphthalene-1-carbonitrile (20.0 g, 79.6 mmol, 1.00 eq.) in acetonitrile (300 mL) was added N-iodosuccinimide (89.5 g, 398 mmol, 5.00 eq.). The mixture was stirred at 80° C. for 12 hours. After such time the mixture was concentrated and the residue was triturated with methyl alcohol (100 mL) at 25° C. for 30 min and the mixture filtered and dried to give 3-fluoro-2-(4-iodo-2-methyl-pyrazol-3-yl)naphthalene-1-carbonitrile (25.2 g, 66.8 mmol, 84% yield) as a yellow solid. LCMS [M+1]⁺=378.0; ¹H NMR (400 MHz, CDCl₃) δ=8.35 (br d, J=8.4 Hz, 1H), 7.98 (br d, J=8.4 Hz, 1H), 7.92 (d, J=9.2 Hz, 1H), 7.82-7.74 (m, 2H), 7.74-7.67 (m, 1H), 3.92-3.82 (m, 3H).

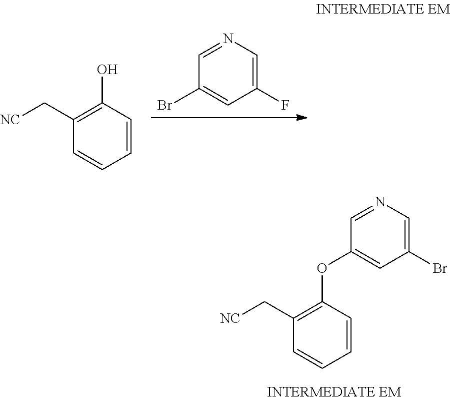

A mixture of 2-(2-hydroxyphenyl)acetonitrile (182 mg, 1.36 mmol, 1.20 eq.), 3-bromo-5-fluoro-pyridine (200 mg, 1.14 mmol, 1.00 eq.), potassium carbonate (393 mg, 2.84 mmol, 2.50 eq.) in DMF (10 mL) was was stirred at 75° C. for 3 hours under a nitrogen atmosphere. The mixture was concentrated and the residue was purified by prep-TLC (SiO₂, petroleum ether:ethyl acetate 15%) to give 2-[2-[(5-bromo-3-pyridyl)oxy]phenyl]acetonitrile (200 mg, 0.43 mmol, 38% yield) as a yellow oil. LCMS [M+1]⁺=289.0.

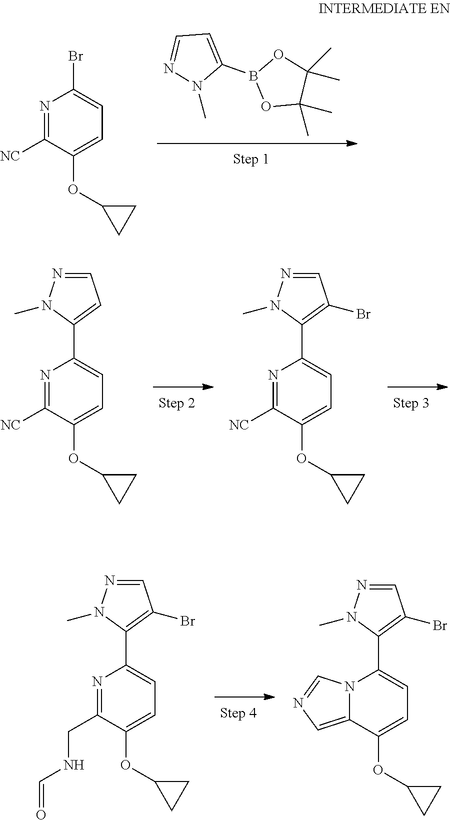 [SMILES: N#Cc1nc(Br)ccc1OC1CC1 ; Cn1nccc1B1OC(C)(C)C(C)(C)O1 ; Cn1nccc1-c1ccc(OC2CC2)c(C#N)n1 ; Cn1ncc(Br)c1-c1ccc(OC2CC2)c(C#N)n1 ; Cn1ncc(Br)c1-c1ccc(OC2CC2)c(CNC=O)n1 ; Cn1ncc(Br)c1-c1ccc(OC2CC2)c2cncn12]

Step 1: To a solution of 6-bromo-3-cyclopropoxypicolinonitrile (800 mg, 3.35 mmol, 1.00 eq.) and 1-methyl-5-(4,4,5,5-tetramethyl-1,3,2-dioxaborolan-2-yl)-1H-pyrazole (732 mg, 3.51 mmol, 1.05 eq.) in dioxane (20 mL) and water (0.4 mL) was added di-tert-butyl(cyclopentyl)phosphane-dichloropalladium-iron (218 mg, 0.335 mmol, 0.10 eq.) and sodium carbonate (709 mg, 6.69 mmol, 2.00 eq.) at 25° C. The mixture was degassed and purged with nitrogen for 3 times, and then stirred at 80° C. for 2 hours. After such time the reaction mixture was quenched with water (20 mL) and then extracted with ethyl acetate (30 mL×3). The combined organic layers were washed with brine (20 mL), dried over anhydrous sodium sulfate, filtered, concentrated and the residue was purified by flash silica gel chromatography (SiO₂, petroleum ether:ethyl acetate 0-10%) to give 3-cyclopropoxy-6-(1-methyl-1H-pyrazol-5-yl)picolinonitrile (750 mg, 2.97 mmol, 89/a yield) as a white solid. ¹H NMR (400 MHz, CD₃OD) δ=7.77 (s, 2H), 7.51 (d, J=2.0 Hz, 1H), 6.57 (d, J=2.0 Hz, 1H), 4.22 (s, 3H), 3.96-3.91 (m, 1H), 0.98-0.92 (m, 4H).

Step 2: To a solution of 3-cyclopropoxy-6-(1-methyl-1H-pyrazol-5-yl)picolinonitrile (650 mg, 2.71 mmol, 1.00 eq.) in acetonitrile (20 mL) was added N-bromosuccinimide (723 mg, 4.06 mmol, 1.50 eq.) at 0° C. and the mixture was stirred at 25° C. for 16 hours. The reaction mixture was quenched with water (2 mL) and extracted with ethyl acetate (3 mL×3). The combined organic layers were washed with brine (10 mL), dried over anhydrous sodium sulfate, filtered, concentrated and the residue purified by prep-TLC (SiO₂, petroleum ether:ethyl acetate=1:1) to give 6-(4-bromo-1-methyl-1H-pyrazol-5-yl)-3-cyclopropoxypicolinonitrile (1.1 g, crude) as a yellow solid. LCMS [M+1]⁺=318.9/320.9; ¹H NMR (400 MHz, CDCl₃) δ=8.00 (d, J=8.8 Hz, 1H), 7.84 (d, J=9.2 Hz, 1H), 7.52 (s, 1H), 4.05 (s, 3H), 3.99-3.92 (m, 1H), 0.99-0.92 (m, 4H).

Step 3: To a solution of 6-(4-bromo-1-methyl-1H-pyrazol-5-yl)-3-cyclopropoxypicolinonitrile (300 mg, 0.94 mmol, 1.00 eq.) in THF (20 mL) was added DIBAL-H (1.00 M, 5.64 mL, 6.00 eq.) at 25° C. and the mixture was stirred at 25° C. for 3 hours. After such time the reaction was quenched by the addition of sodium thiosulfate solution (20 mL). The mixture was extracted with ethyl acetate (30 mL×3) and the combined organic layers were washed with brine (20 mL), dried over anhydrous sodium sulfate, filtered, concentrated and the residue was purified by prep-TLC (SiO₂, petroleum ether:ethyl acetate 1:1) to give (6-(4-bromo-1-methyl-1H-pyrazol-5-yl)-3-cyclopropoxypyridin-2-yl) methanamine (160 mg, 0.495 mmol) as a yellow solid. A mixture of (6-(4-bromo-1-methyl-1H-pyrazol-5-yl)-3-cyclopropoxypyridin-2-yl) methanamine (160 mg, 0.495 mmol, 1.00 eq.) in ethyl formate (3 mL) was stirred at 25° C. for 2 hours followed by the addition of water (2 mL). The mixture was then extracted with ethyl acetate (3 mL×3) and the combined organic layers were washed with brine (10 mL), dried over anhydrous sodium sulfate, filtered, concentrated and the residue was purified by prep-TLC (SiO₂, petroleum ether:ethyl acetate 0-10/6) to give N-((6-(4-bromo-1-methyl-1H-pyrazol-5-yl)-3-cyclopropoxypyridin-2-yl) methyl) formamide (140 mg, 0.359 mmol, 73% yield) as a yellow solid. LCMS [M+1]⁺=351.0/353.0; ¹H NMR (400 MHz, CDCl₃) δ=8.36 (s, 1H), 7.68-7.61 (m, 2H), 7.55-7.52 (m, 1H), 7.03 (br s, 1H), 4.62 (d, J=4.4 Hz, 2H), 3.99 (s, 3H), 3.85 (tt, J=3.2, 5.6 Hz, 1H), 0.92-0.85 (m, 4H).

Step 4: To a solution of N-((6-(4-bromo-1-methyl-1H-pyrazol-5-yl)-3-cyclopropoxypyridin-2-yl) methyl) formamide (140 mg, 0.359 mmol, 1.00 eq) and diisopropylethylamine (104 mg, 0.80 mmol, 0.14 mL, 2.00 eq.) in dichloromethane (8 mL) was added Tf₂O (225 mg, 0.078 mmol, 0.13 mL, 2.00 eq.) at −40° C. then allowed to warm to ambient temperature and stirred at 25° C. for 6 hours. After such time water (2 mL) was added and the mixture extracted with ethyl acetate (3 mL×3). The combined organic layers were washed with brine (10 mL), dried over anhydrous sodium sulfate, filtered, concentrated and the residue was purified by prep-TLC (SiO₂, petroleum ether:ethyl acetate 0-10%) to give 5-(4-bromo-1-methyl-1H-pyrazol-5-yl)-8-cyclopropoxyimidazo[1,5-a]pyridine (110 mg, 0.314 mmol, 79% yield) as a yellow solid. LCMS [M+1]⁺=332.9/334.9; ¹H NMR (400 MHz, CDCl₃) δ=7.72-7.60 (m, 3H), 6.61 (d, J=7.6 Hz, 1H), 6.49 (d, J=7.4 Hz, 1H), 3.98-3.92 (m, 1H), 3.77 (s, 3H), 0.98-0.87 (m, 4H).

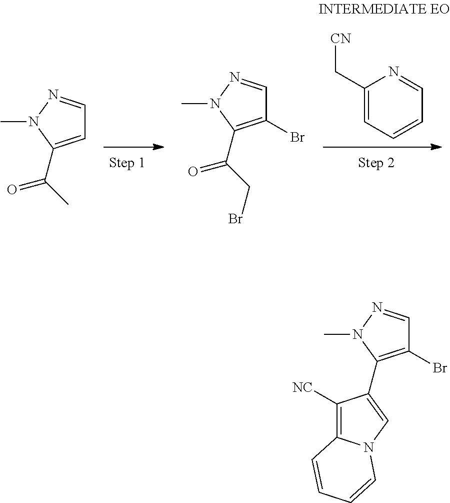

Step 1: To a solution of 1-(2-methylpyrazol-3-yl)ethanone (400 mg, 3.22 mmol, 1.00 eq.) in THF (6 mL) was added 1-bromopyrrolidine-2,5-dione (1.43 g, 8.06 mmol, 2.50 eq.) and the mixture was stirred at 25° C. for 12 hours. After such time the reaction mixture was concentrated and the residue purified by prep-TLC (SiO₂, petroleum ether:ethyl acetate 20/a) to give 2-bromo-1-(4-bromo-2-methyl-pyrazol-3-yl)ethanone (800 mg, 2.84 mmol, 88% yield) as a yellow oil. LCMS [M+1]⁺=282.9; ¹H NMR (400 MHz, CDCl₃) δ=7.54 (s, 1H), 4.62 (s, 2H), 4.16 (s, 3H).

Step 2: A solution of 2-bromo-1-(4-bromo-2-methyl-pyrazol-3-yl)ethanone (400 mg, 1.42 mmol, 1.00 eq.) and 2-(2-pyridyl)acetonitrile (335 mg, 2.84 mmol, 0.31 mL, 2.00 eq.) in acetonitrile (6 mL) was stirred at 70° C. for 11 hours followed by the addition of triethylamine (431 mg, 4.26 mmol, 0.59 mL, 3.00 eq.) and stirred at 70° C. for a further 1 hour. The reaction mixture was then concentrated and the residue purified by prep-TLC (SiO₂, petroleum ether:ethyl acetate 30%) to give 2-(4-bromo-2-methyl-pyrazol-3-yl)indolizine-1-carbonitrile (100 mg, 0.332 mmol, 23% yield) as a yellow solid. LCMS [M+1]⁺=301.0; ¹H NMR (400 MHz, CDCl₃) δ=8.10 (d, J=6.8 Hz, 1H), 7.72 (d, J=9.2 Hz, 1H), 7.59 (s, 1H), 7.44 (s, 1H), 7.22-7.15 (m, 1H), 6.89 (t, J=6.4 Hz, 1H), 3.96 (s, 3H).

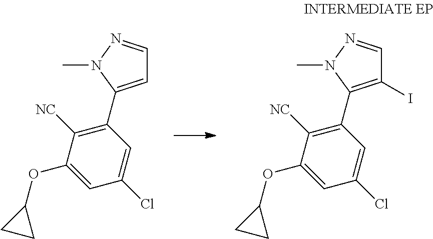 [SMILES: Cn1nccc1-c1cc(Cl)cc(OC2CC2)c1C#N ; Cn1ncc(I)c1-c1cc(Cl)cc(OC2CC2)c1C#N]

To a solution of 4-chloro-2-(cyclopropoxy)-6-(2-methylpyrazol-3-yl)benzonitrile (150 mg, 0.55 mmol, 1.00 eq.) in acetic acid (1.0 mL) was added N-iodosuccinimide (247 mg, 1.10 mmol, 2.00 eq.) and the mixture was stirred at 25° C. for 1 hour. The reaction mixture was then diluted with ethyl acetate (30 mL) and washed with brine (30 mL×3), dried over anhydrous sodium sulfate, filtered, concentrated and the residue purified by prep-TLC (SiO₂, petroleum ether:ethyl acetate 20%) to give 4-chloro-2-(cyclopropoxy)-6-(4-iodo-2-methyl-pyrazol-3-yl)benzonitrile (135 mg, 0.33 mmol, 61% yield) as a yellow solid. LCMS [M+H]⁺=399.9; ¹H NMR (400 MHz, CDCl₃) δ=7.53 (s, 1H), 7.41 (d, J=2.0 Hz, 1H), 6.94 (d, J=2.0 Hz, 1H), 3.90-3.79 (m, 1H), 3.75 (s, 3H), 0.99-0.76 (m, 4H).

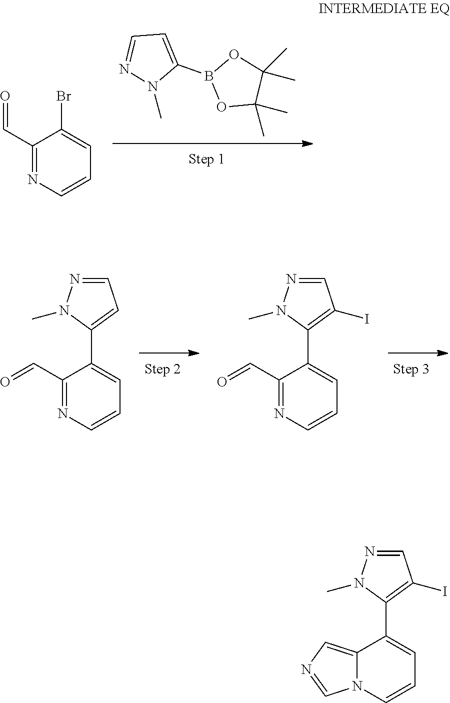 [SMILES: O=Cc1ncccc1Br ; Cn1nccc1B1OC(C)(C)C(C)(C)O1 ; Cn1nccc1-c1cccnc1C=O ; Cn1ncc(I)c1-c1cccnc1C=O ; Cn1ncc(I)c1-c1cccn2cncc12]

Step 1: A mixture of 3-bromopyridine-2-carbaldehyde (1.00 g, 5.38 mmol, 1.00 eq.), 1-methyl-5-(4,4,5,5-tetramethyl-1,3,2-dioxaborolan-2-yl)pyrazole (1.12 g, 5.38 mmol, 1.00 eq.), sodium bicarbonate (1.13 g, 13.4 mmol, 0.52 mL, 2.50 eq.) and triphenyl phosphine (141 mg, 0.54 mmol, 0.10 eq.) in DMF (10 mL) and water (2 mL) was degassed and purged with nitrogen 3 times, then palladium acetate (60 mg, 0.27 mmol, 0.05 eq.) was added to the mixture and stirred at 80° C. for 16 hours. After such time the reaction solution was filtered, poured into water (2 mL) and extracted with ethyl acetate (10 mL×3). The combined organic layers were washed with brine (5 mL), dried over anhydrous sodium sulfate, filtered, concentrated and the residue was purified by column chromatography (SiO₂, petroleum ether:ethyl acetate 20-100%) to give 3-(2-methylpyrazol-3-yl)pyridine-2-carbaldehyde (637 mg, 3.40 mmol, 63% yield) as a brown solid. LCMS [M+1]⁺=188.0; ¹H NMR (400 MHz, CDCl₃) δ=10.14-10.06 (m, 1H), 8.93 (dd, J=1.6, 4.8 Hz, 1H), 7.79 (dd, J=1.2, 7.6 Hz, 1H), 7.64-7.58 (m, 2H), 6.30 (d, J=2.0 Hz, 1H), 3.67 (s, 3H).

Step 2: To a solution of 3-(2-methylpyrazol-3-yl)pyridine-2-carbaldehyde (200 mg, 1.07 mmol, 1.00 eq.) in acetonitrile (5 mL) was added N-iodosuccinimide (480 mg, 2.14 mmol, 2.00 eq.) and the mixture was stirred at 20° C. for 16 hours. The reaction was diluted by ethyl acetate (35 mL), washed with saturated sodium thiosulfate (5 mL×2), dried over anhydrous sodium sulfate, filtered, concentrated and the residue was purified by column chromatography (SiO₂, petroleum ether:ethyl acetate 20-100%) to give 3-(4-iodo-2-methyl-pyrazol-3-yl)pyridine-2-carbaldehyde (290 mg, 0.93 mmol, 87% yield) as a white solid. LCMS [M+1]⁺=313.8; ¹H NMR (400 MHz, CDCl₃) δ=10.07 (s, 1H), 8.97 (dd, J=1.6, 4.8 Hz, 1H), 7.79-7.73 (m, 1H), 7.68 (dd, J=4.8, 7.6 Hz, 1H), 7.63 (s, 1H), 3.69 (s, 3H).

Step 3: To a solution of 3-(4-iodo-2-methyl-pyrazol-3-yl)pyridine-2-carbaldehyde (290 mg, 0.93 mmol, 1.00 eq.) in DMF (5 mL) was added 2-aminoacetic acid (77 mg, 1.02 mmol, 1.10 eq.), iodine (235 mg, 0.93 mmol, 0.18 mL, 1.00 eq.) and sodium bicarbonate (155 mg, 1.85 mmol, 2.00 eq.). Then the mixture was then stirred at 60° C. for 6 hours. The reaction mixture was diluted with ethyl acetate (35 mL), washed with saturated sodium thiosulfate solution (2 mL×3), dried over anhydrous sodium sulfate, filtered, concentrated and the residue was purified by column chromatography (SiO₂, petroleum ether:ethyl acetate 20-100%) to give 8-(4-iodo-2-methyl-pyrazol-3-yl)imidazo[1,5-a]pyridine (100 mg, 0.31 mmol, 33% yield) as a yellow gum. LCMS [M−1]⁻=324.9; ¹H NMR (400 MHz, CDCl₃) δ=8.24 (s, 1H), 8.05 (d, J=6.4 Hz, 1H), 7.65 (s, 1H), 7.19 (s, 1H), 6.78-6.69 (m, 2H), 3.82 (s, 3H).

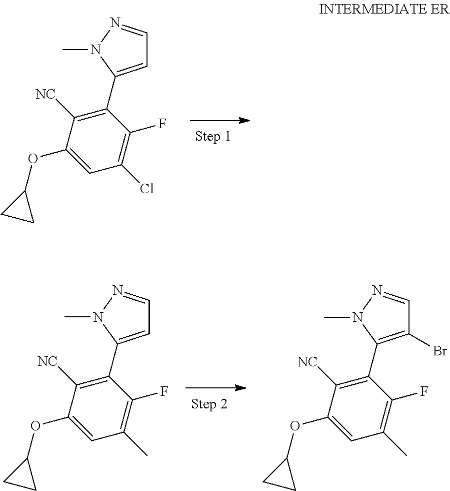 [SMILES: Cn1nccc1-c1c(F)c(Cl)cc(OC2CC2)c1C#N ; Cc1cc(OC2CC2)c(C#N)c(-c2ccnn2C)c1F ; Cc1cc(OC2CC2)c(C#N)c(-c2c(Br)cnn2C)c1F]

Step 1: A mixture of 4-chloro-6-(cyclopropoxy)-3-fluoro-2-(2-methylpyrazol-3-yl)benzonitrile (200 mg, 0.69 mmol, 1.00 eq.), methylboronic acid (205 mg, 3.43 mmol, 5.00 eq.), di-tert-butyl(cyclopentyl)phosphane-dichloropalladium-iron (45 mg, 0.069 mmol, 0.10 eq.) and potassium carbonate (284 mg, 2.06 mmol, 3.00 eq.) in dioxane (2 mL) was degassed, purged with nitrogen 3 times and stirred at 100° C. for 2 hours. The mixture was then concentrated and purified by prep-TLC (SiO₂, petroleum ether:ethyl acetate 25%) to give 6-(cyclopropoxy)-3-fluoro-4-methyl-2-(2-methylpyrazol-3-yl)benzonitrile (35 mg, 0.10 mmol, 15% yield) as a yellow oil. LCMS [M+1]⁺=274.3; ¹H NMR (400 MHz, CDCl₃) δ=7.60 (d, J=2.0 Hz, 1H), 7.24 (d, J=6.0 Hz, 1H), 6.46 (d, J=2.0 Hz, 1H), 3.86 (td, J=2.8, 5.6 Hz, 1H), 3.80 (s, 3H), 2.43 (d, J=2.0 Hz, 3H), 0.93-0.88 (m, 4H).

Step 2: A mixture of 6-(cyclopropoxy)-3-fluoro-4-methyl-2-(2-methylpyrazol-3-yl)benzonitrile (35 mg, 0.13 mmol, 1.00 eq.), N-bromosuccinimide (46 mg, 0.26 mmol, 2.00 eq.) in acetonitrile (3 mL) was stirred at 40° C. for 2 hours under a nitrogen atmosphere. The mixture was then concentrated and the residue was purified by prep-TLC (SiO₂, petroleum ether:ethyl acetate 25%) to give 2-(4-bromo-2-methyl-pyrazol-3-yl)-6-(cyclopropoxy)-3-fluoro-4-methyl-benzonitrile (25 mg, 0.040 mmol, 31% yield) as a white solid. LCMS [M+1]⁺=352.0; ¹H NMR (400 MHz, CDCl₃) δ=7.59 (d, J=2.8 Hz, 1H), 7.31 (br d, J=2.8 Hz, 1H), 3.90-3.83 (m, 1H), 3.78 (d, J=2.8 Hz, 3H), 2.44 (br s, 3H), 0.92 (br dd, J=3.2, 6.4 Hz, 4H).

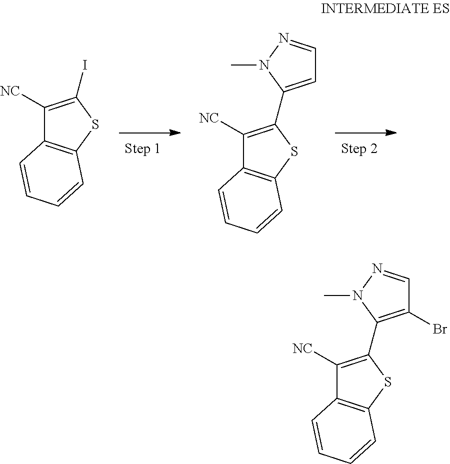 [SMILES: N#Cc1c(I)sc2ccccc12 ; Cn1nccc1-c1sc2ccccc2c1C#N ; Cn1ncc(Br)c1-c1sc2ccccc2c1C#N]

Step 1: To a mixture of 2-iodobenzothiophene-3-carbonitrile (280 mg, 0.98 mmol, 1.00 eq.), sodium carbonate (312 mg, 2.95 mmol, 3.00 eq.) and 1-methyl-5-(4,4,5,5-tetramethyl-1,3,2-dioxaborolan-2-yl)pyrazole (409 mg, 1.96 mmol, 2.00 eq.) in dioxane (4 mL) and water (1 mL) was added di-tert-butyl(cyclopentyl)phosphane-dichloropalladium-iron (64 mg, 0.098 mmol, 0.10 eq.) and sodium carbonate (312 mg, 2.95 mmol, 3.00 eq.) and the mixture was stirred at 80° C. for 2 hours. After such time water (5 mL) was added and the mixture extracted with ethyl acetate (10 mL×3). The combined organic phase were washed with brine (10 mL), dried over anhydrous sodium sulfate, filtered, concentrated and the residue was purified by prep-TLC (SiO₂, petroleum ether:ethyl acetate 10%) to give 2-(2-methylpyrazol-3-yl)benzothiophene-3-carbonitrile (150 mg, 64% yield) as a yellow solid. ¹H NMR (400 MHz, CDCl₃) δ (ppm)=8.06-8.01 (m, 1H), 7.94-7.89 (m, 1H), 7.64-7.61 (m, 1H), 7.61-7.51 (m, 2H), 6.79 (d, J=1.6 Hz, 1H), 4.09 (s, 3H).

Step 2: To a mixture of 2-(2-methylpyrazol-3-yl)benzothiophene-3-carbonitrile (150 mg, 0.63 mmol, 1.00 eq.) in acetonitrile (2 mL) was added N-bromosuccinimide (112 mg, 0.63 mmol, 1.00 eq.) and the mixture was stirred at 25° C. for 12 hours. After such time the reaction mixture was added to a saturated sodium bicarbonate solution (5 mL) and extracted with ethyl acetate (10 mL×3). The combined organic phases were washed with brine (15 mL), dried over anhydrous sodium sulfate, filtered, concentrated and the residue was purified by column chromatography (SiO₂, petroleum ether:ethyl acetate 0-100%) to give 2-(4-bromo-2-methyl-pyrazol-3-yl)benzothiophene-3-carbonitrile (160 mg, 75% yield) as a yellow solid. LCMS [M+1]⁺=319.9: ¹H NMR (400 MHz, CDCl₃) δ (ppm)=8.10-8.05 (m, 1H), 7.98-7.93 (m, 1H), 7.65 (s, 1H), 7.63-7.56 (m, 2H), 3.98 (s, 3H).

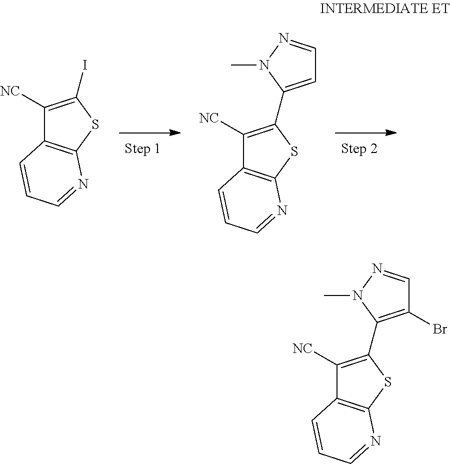 [SMILES: N#Cc1c(I)sc2ncccc12 ; Cn1nccc1-c1sc2ncccc2c1C#N ; Cn1ncc(Br)c1-c1sc2ncccc2c1C#N]

Intermediate ES, 2-(4-bromo-2-methyl-pyrazol-3-yl)thieno[2,3-b]pyridine-3-carbonitrile, was prepared as a yellow solid (80.0 mg, 0.25 mmol, 22% yield over 2 steps) from thieno[2,3-b]pyridine-3-carbonitrile in steps following the procedure described for Intermediate ER. LCMS [M+1]+=320.9; ¹H NMR (400 MHz, CDCl₃) δ (ppm)=8.78 (dd, J=1.6, 4.4 Hz, 1H), 8.34 (dd, J=1.6, 8.0 Hz, 1H), 7.66 (s, 1H), 7.58 (dd, J=4.4, 8.0 Hz, 1H), 3.99 (s, 3H).

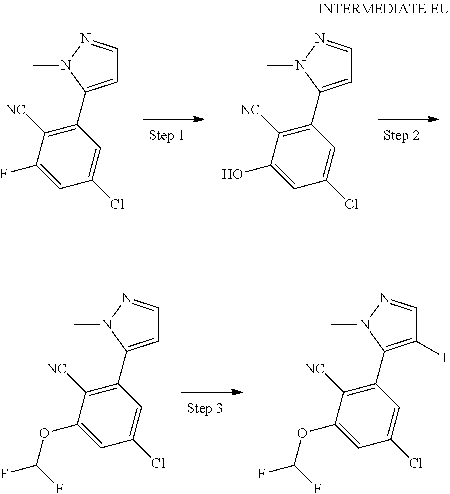 [SMILES: Cn1nccc1-c1cc(Cl)cc(F)c1C#N ; Cn1nccc1-c1cc(Cl)cc(O)c1C#N ; Cn1nccc1-c1cc(Cl)cc(OC(F)F)c1C#N ; Cn1ncc(I)c1-c1cc(Cl)cc(OC(F)F)c1C#N]

Step 1: To a solution of 2-methylsulfonylethanol (569 mg, 4.58 mmol, 1.20 eq.) in DMF (30 mL) was added sodium hydride (183 mg, 4.58 mmol, 60.0% purity, 1.20 eq.) at 0° C. After stirring for 0.5 hour 4-chloro-2-fluoro-6-(2-methylpyrazol-3-yl)benzonitrile (900 mg, 3.82 mmol, 1.00 eq.) in DMF (5 mL) was added in a dropwise fashion at 0° C. The reaction mixture was stirred at 25° C. for 1 hour. After such time the mixture was diluted with water (100 mL), extracted with ethyl acetate (100 mL×3), the aqueous phase adjusted to pH 1 with HCl (10 mL) and further extracted with ethyl acetate (100 mL×3). The combined organic layers were dried over anhydrous sodium sulfate, filtered and concentrated to give 4-chloro-2-hydroxy-6-(2-methylpyrazol-3-yl)benzonitrile (455 mg, crude) as a yellow solid which used into the next step without further purification. LCMS [M]⁺=234.1; ¹H NMR (400 MHz, DMSO-d₆) δ=11.96 (br s, 1H), 7.54 (d, J=2.0 Hz, 1H), 7.16 (d, J=2.0 Hz, 1H), 7.12 (d, J=2.0 Hz, 1H), 6.50 (d, J=2.0 Hz, 1H), 3.76 (s, 3H).

Step 2: To a solution of 4-chloro-2-hydroxy-6-(2-methylpyrazol-3-yl)benzonitrile (150 mg, 0.642 mmol, 1.00 eq.) and sodium 2-chloro-2,2-difluoro-acetate (392 mg, 2.57 mmol, 4.00 eq.) in DMF (2 mL) and water (0.2 mL) was added cesium carbonate (314 mg, 0.96 mmol, 1.50 eq.). The mixture was stirred at 100° C. for 1 hour. The reaction mixture was then quenched by addition water (40 mL) and extracted with ethyl acetate (20 mL×3). The combined organic layers were washed with brine (20 mL), dried over anhydrous sodium sulfate, filtered, concentrated and the residue was purified by prep-TLC (SiO₂, petroleum ether:ethyl acetate 25%) to give 4-chloro-2-(difluoromethoxy)-6-(2-methylpyrazol-3-yl) benzonitrile (85 mg, 0.30 mmol, 47% yield) as a yellow solid. LCMS [M]⁺=284.0; ¹H NMR (400 MHz, DMSO-d₆) S (ppm)=7.76 (s, 1H), 7.74 (d, J=2.0 Hz, 1H), 7.72-7.71 (m, 1H), 7.58 (d, J=2.0 Hz, 1H), 7.54 (s, 1H), 7.36 (s, 1H), 6.60 (d, J=2.0 Hz, 1H), 3.80 (s, 3H).

Step 3: To a solution of 4-chloro-2-(difluoromethoxy)-6-(2-methylpyrazol-3-yl) benzonitrile (85 mg, 0.30 mmol, 1.00 eq.) in acetic acid (2 mL) was added N-iodide succinimide (135 mg, 0.60 mmol, 2.00 eq.). The mixture was stirred at 25° C. for 1 hour then the reaction mixture was quenched by addition of water (40 mL) and extracted with ethyl acetate (20 mL×3). The combined organic layers were washed with brine (40 mL), dried over anhydrous sodium sulfate, filtered, concentrated and the residue was purified by prep-TLC (SiO₂, petroleum ether: ethyl acetate 20%) to give 4-chloro-2-(difluoromethoxy)-6-(4-iodo-2-methyl-pyrazol-3-yl) benzonitrile (90 mg, 0.22 mmol, 73% yield) as a yellow solid. LCMS [M+H]⁺=409.9; ¹H NMR (400 MHz, CD₃OD-d₄) δ (ppm)=7.72-7.68 (m, 1H), 7.67 (s, 1H), 7.52 (d, J=2.0 Hz, 1H), 7.42-7.02 (m, 1H), 3.83 (s, 3H).

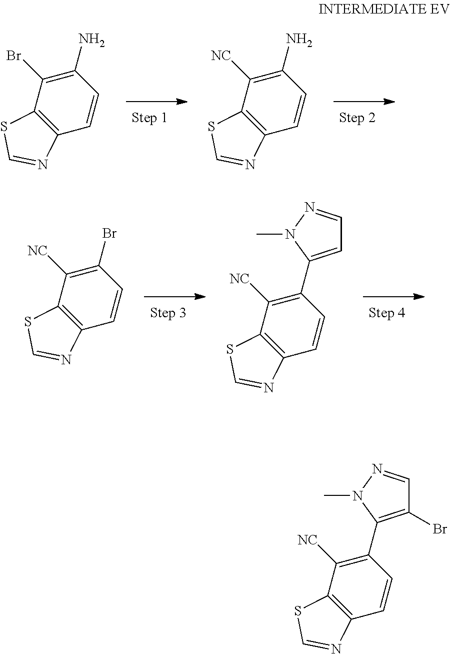 [SMILES: Nc1ccc2ncsc2c1Br ; N#Cc1c(N)ccc2ncsc12 ; N#Cc1c(Br)ccc2ncsc12 ; Cn1nccc1-c1ccc2ncsc2c1C#N ; Cn1ncc(Br)c1-c1ccc2ncsc2c1C#N]

Step 1: A mixture of 7-bromo-1,3-benzothiazol-6-amine (2.00 g, 8.73 mmol, 1.00 eq.), zinc cyanide (1.54 g, 13.1 mmol, 1.50 eq.), Pd₂(dba)₃ (80 mg, 0.87 mmol, 0.01 eq.), DPPF (97 mg, 0.175 mmol, 0.02 eq.) and zinc powder (5.7 mg, 0.087 mmol, 0.01 eq.) in DMF (20 mL) was degassed, purged with nitrogen 3 times, and then stirred at 140° C. for 16 hours. After such time the reaction mixture was extracted with ethyl acetate 150 mL (50 mL×3) and the combined organic layers were washed with brine (50 mL), dried over anhydrous sodium sulfate, filtered, concentrated and the formed residue purified by column chromatography (SiO₂, petroleum ether:ethyl acetate 10-25%) to give 6-amino-1,3-benzothiazole-7-carbonitrile (1.05 g, 4.66 mmol, 53% yield) as a yellow solid. LCMS [M+1]⁺=176.1; ¹H NMR (400 MHz, CDCl₃-d) δ=8.79 (s, 1H), 8.05 (d, J=8.8 Hz, 1H), 6.90 (d, J=8.8 Hz, 1H), 4.69 (br s, 2H).

Step 2: A mixture of 6-amino-1,3-benzothiazole-7-carbonitrile (500 mg, 2.85 mmol, 1.00 eq.), para-toluenesulfonic acid (590 mg, 3.42 mmol, 1.20 eq.), tert-butyl nitrite (353 mg, 3.42 mmol, 407 μL, 1.20 eq.), tetrabutylammonium bromide (1.84 g, 5.71 mmol, 2.00 eq.) and copper bromide (64 mg, 0.286 mmol, 0.10 eq.) in acetonitrile (15 mL) was degassed with nitrogen and stirred at 25° C. for 6 hours. The mixture was then concentrated under reduced pressure and the residue was purified by column chromatography (SiO₂, petroleum ether:ethyl acetate 10-25%) to give 6-bromo-1,3-benzothiazole-7-carbonitrile (300 mg, 1.25 mmol, 44% yield) as a yellow solid. LCMS [M+1]⁺=240.9; ¹H NMR (400 MHz, CDCl₃) δ=9.11 (s, 1H), 8.20 (d, J=8.8 Hz, 1H), 7.81 (d, J=8.8 Hz, 1H).

Step 3: A mixture of 1-methyl-5-(4,4,5,5-tetramethyl-1,3,2-dioxaborolan-2-yl)pyrazole (261 mg, 1.25 mmol, 1.00 eq.), 6-bromo-1,3-benzothiazole-7-carbonitrile (300 mg, 1.25 mmol, 1.00 eq.), di-tert-butyl(cyclopentyl)phosphane;dichloropalladium iron (82 mg, 0.125 mmol, 0.10 eq.) and sodium bicarbonate (316 mg, 3.76 mmol, 3.00 eq.) in dioxane (10 mL) and water (2 mL) was degassed with nitrogen and then stirred at 80° C. for 3 hours under a nitrogen atmosphere. The mixture was then concentrated and the residue purified by column chromatography (SiO₂, petroleum ether:ethyl acetate 5-25%) to give 6-(2-methylpyrazol-3-yl)-1,3-benzothiazole-7-carbonitrile (280 mg, 0.89 mmol, 71% yield) as a yellow solid. LCMS [M+1]⁺=241.0; ¹H NMR (400 MHz, CDCl₃-d) δ=9.21 (s, 1H), 8.43 (d, J=8.4 Hz, 1H), 7.66-7.58 (m, 2H), 6.58 (d, J=2.0 Hz, 1H), 3.91 (s, 3H).

Step 4: A mixture of 6-(2-methylpyrazol-3-yl)-1,3-benzothiazole-7-carbonitrile (140 mg, 0.58 mmol, 1.00 eq.), N-bromosuccinimide (207 mg, 1.17 mmol, 2.00 eq.) in acetonitrile (3 mL) was stirred at 40° C. for 2 hours under a nitrogen atmosphere. The mixture was then concentrated and the residue purified by column chromatography (SiO₂, petroleum ether:ethyl 5-20%) to give 6-(4-bromo-2-methyl-pyrazol-3-yl)-1,3-benzothiazole-7-carbonitrile (300 mg, 0.47 mmol, 81% yield) as a white solid. LCMS [M+1]⁺=321.0; ¹H NMR (400 MHz, CDCl₃-d) δ=9.25 (s, 1H), 8.48 (d, J=8.4 Hz, 1H), 7.65 (s, 1H), 7.61 (d, J=8.4 Hz, 1H), 3.85 (s, 3H).

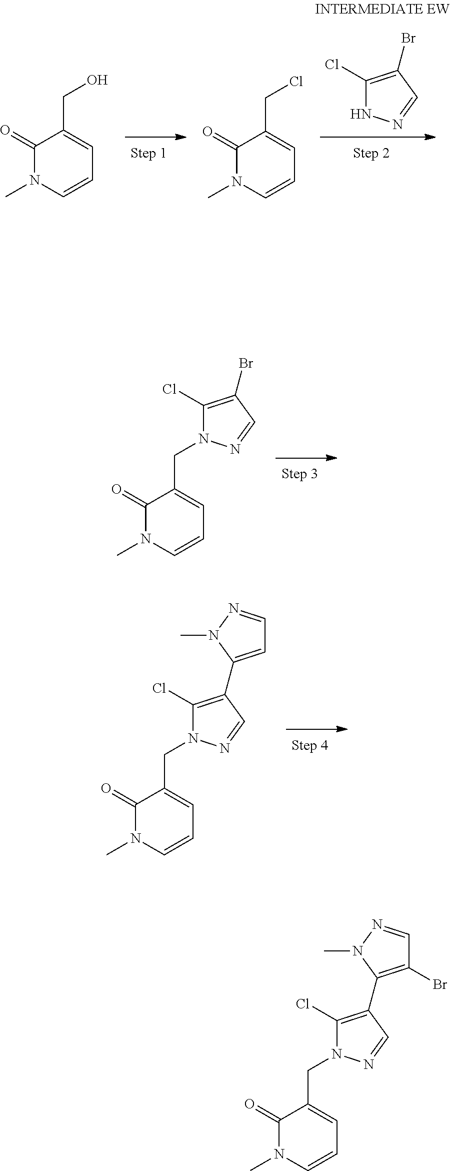

Step 1: To a solution of 3-(hydroxymethyl)-1-methylpyridin-2 (1H)-one (650 mg, 4.67 mmol, 1.00 eq.) in dichloromethane (15 mL) was added thionyl chloride (667 mg, 5.61 mmol, 407 μL, 1.20 eq.) and the mixture was stirred at 25° C. for 2 hrs. The mixture was concentrated in vacuum to give 3-(chloromethyl)-1-methyl-pyridin-2-one (650 mg, crude) as a white solid.

Step 2: To a solution of 3-(chloromethyl)-1-methyl-pyridin-2-one (650 mg, 4.12 mmol, 1.00 eq.), 4-bromo-5-chloro-1H-pyrazole (747 mg, 4.12 mmol, 1.00 eq.) in acetonitrile (20 mL) was added potassium carbonate (683 mg, 4.94 mmol, 1.20 eq.) and the mixture was stirred at 80° C. for 12 hours. The reaction was then quenched with water (100 mL) and extracted with ethyl acetate (50 mL×3). The combined organic phases were washed with brine (50 mL), dried over anhydrous sodium sulfate, filtered and concentrated in vacuo and the residue was purified by column chromatography (SiO₂, petroleum ether:ethyl acetate 5%) to give 3-[(4-bromo-5-chloro-pyrazol-1-yl)methyl]-1-methyl-pyridin-2-one (270 mg, 0.89 mmol, 51% yield) as a yellow solid. LCMS [M+1]⁺=304.0.

Step 3: To a solution of 3-[(4-bromo-5-chloro-pyrazol-1-yl)methyl]-1-methyl-pyridin-2-one (270 mg, 0.89 mmol, 1.00 eq.), 4-bromo-1-methyl-5-(4,4,5,5-tetramethyl-1,3,2-dioxaborolan-2-yl)pyrazole (512 mg, 1.78 mmol, 2.00 eq.) in dioxane (10 mL) and water (1 mL) was added Pd(dppf)Cl₂ (65 mg, 0.089 mmol, 0.10 eq.) and sodium bicarbonate (150 mg, 1.78 mmol, 69 μL, 2.00 eq.). The mixture was stirred at 110° C. for 10 hours. The reaction mixture was then quenched by addition water (50 mL) and extracted with ethyl acetate (30 mL×3). The combined organic layers were washed with brine (30 mL), dried over anhydrous sodium sulfate, filtered and concentrated under reduced pressure and the residue was purified by prep-HPLC (Phenomenex Gemini-NX 80 mm×40 mm×3 μm; mobile phase: [water (10 mM NH₄HCO₃)-ACN]; B %: 5%-35%, 8 min) to give 3-[[5-chloro-4-(2-methylpyrazol-3-yl)pyrazol-1-yl]methyl]-1-methyl-pyridin-2-one (60.0 mg, 0.198 mmol, 22% yield) as a grey solid. LCMS [M+1]⁺=304.1; ¹H NMR (400 MHz, DMSO-d₆) δ=8.24 (s, 1H), 7.73 (d, J=2.0, 6.8 Hz, 1H), 7.46 (d, J=2.0 Hz, 1H), 7.32 (d, J=5.2 Hz, 1H), 6.39 (d, J=2.0 Hz, 1H), 6.25 (t, J=6.8 Hz, 1H), 5.14 (s, 2H), 3.79 (s, 3H), 3.45 (s, 3H)

Step 4: To a solution of 3-((5′-chloro-2-methyl-1′H,2H-[3,4′-bipyrazol]-1′-yl)methyl)-1-methylpyridin-2 (1H)-one (47.0 mg, 0.155 mmol, 1.00 eq.) in acetonitrile (1 mL) was added N-bromosuccinimide (26 mg, 0.147 mmol, 0.95 eq.). The mixture was stirred at 25° C. for 10 hours. The reaction mixture was then quenched by addition of water (5 mL) and extracted with ethyl acetate (5 mL×3). The combined organic layers were washed with brine (5 mL), dried over anhydrous sodium sulfate, filtered, concentrated and the residue was purified by prep-TLC (SiO₂, dichloromethane:methanol 10%) to give 3-((4-bromo-5′-chloro-2-methyl-1′H,2H-[3,4′-bipyrazol]-1′-yl)methyl)-1-methylpyridin-2 (1H)-one (45 mg, 0.118 mmol, 76% yield) as a white solid. LCMS [M+1]⁺=383.9; ¹H NMR (400 MHz, DMSO-d₆) δ=7.89 (s, 1H), 7.71 (d, J=6.8, 1.2 Hz, 1H), 7.67 (s, 1H), 6.93-6.96 (m, 1H), 6.23 (t, J=6.8 Hz, 1H), 5.22 (s, 2H), 3.74 (s, 3H), 3.47 (s, 3H).

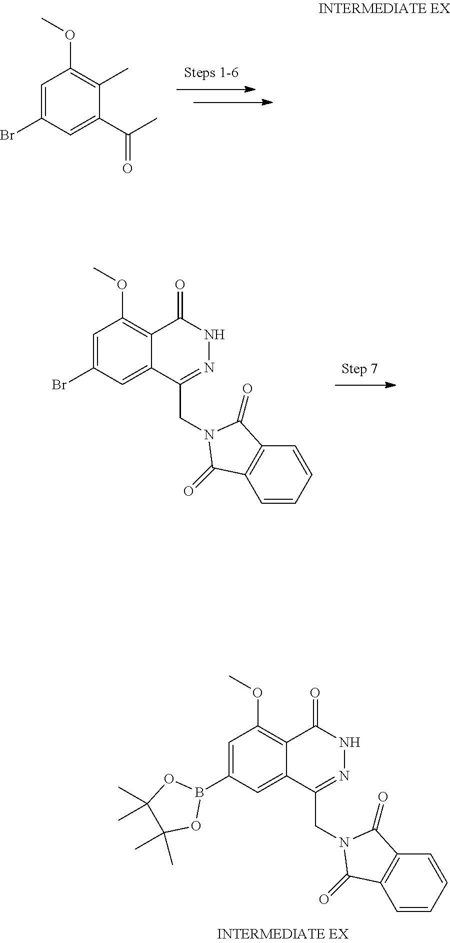 [SMILES: COc1cc(Br)cc(C(C)=O)c1C ; COc1cc(Br)cc2c(CN3C(=O)c4ccccc4C3=O)n[nH]c(=O)c12 ; COc1cc(B2OC(C)(C)C(C)(C)O2)cc2c(CN3C(=O)c4ccccc4C3=O)n[nH]c(=O)c12]

Steps 1-6: 2-((7-bromo-4-oxo-5-(methoxy)-3,4-dihydrophthalazin-1-yl)methyl)isoindoline-1,3-dione was prepared as a white solid (4.22 g, 10.2 mmol, 8% yield over 6 steps) starting from 1-(5-bromo-2-methyl-3-(methoxy)phenyl)ethan-1-one following the same procedure described for the first 6 steps of Intermediate DK. LCMS [M+1]⁺=414.0.

Step 7: A mixture of 2-((7-bromo-4-oxo-5-(methoxy)-3,4-dihydrophthalazin-1-yl)methyl)isoindoline-1,3-dione (4.22 g, 10.2 mmol, 1.0 eq.), bis(pinacolato)diboron (3.88 g, 15.3 mmol, 1.5 eq.), Pd(dppf)Cl₂·CH₂Cl₂ (745 mg, 1.02 mmol, 0.1 eq.) and potassium acetate (3.00 g, 30.6 mmol, 3.0 eq.) in dioxane (60 mL) was degassed and purged with nitrogen 3 times, and stirred at 100° C. for 1 hour. The mixture was then concentrated, and the residue triturated with methanol, filtered and dried to give Intermediate EX as a grey solid (2.01 g, 43% yield). LCMS [M+1]⁺=380.1 (boronic acid from loss of pinicol).

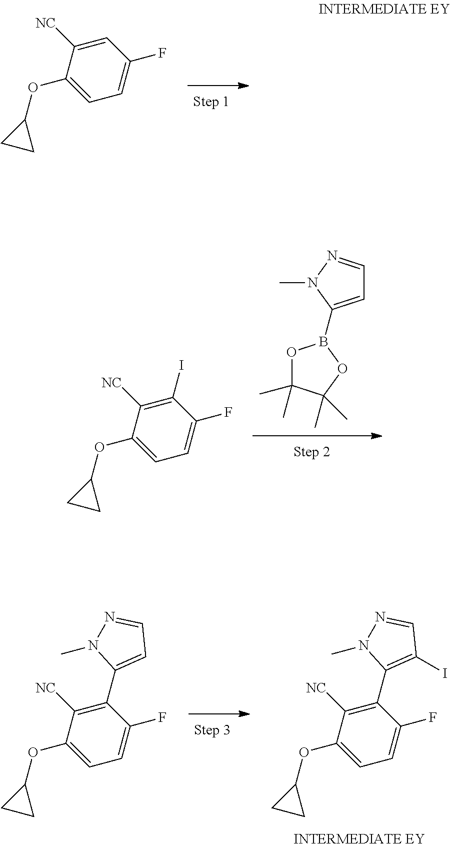

6-cyclopropoxy-3-fiuoro-2-(4-iodo-1-methyl-1H-pyrazol-5-yl)benzonitrile, intermediate EY, was prepared as a white solid (120 mg, 0.30 mmol, 20% over 3 steps) following the procedure described for the preparation of Intermediate DQ. LCMS [M+1]⁺=383.8; ¹H NMR (400 MHz, CDCl₃) δ=7.65 (s, 1H), 7.51-7.40 (m, 2H), 3.93-3.87 (m, 1H), 3.83 (s, 3H), 1.00-0.89 (m, 4H).

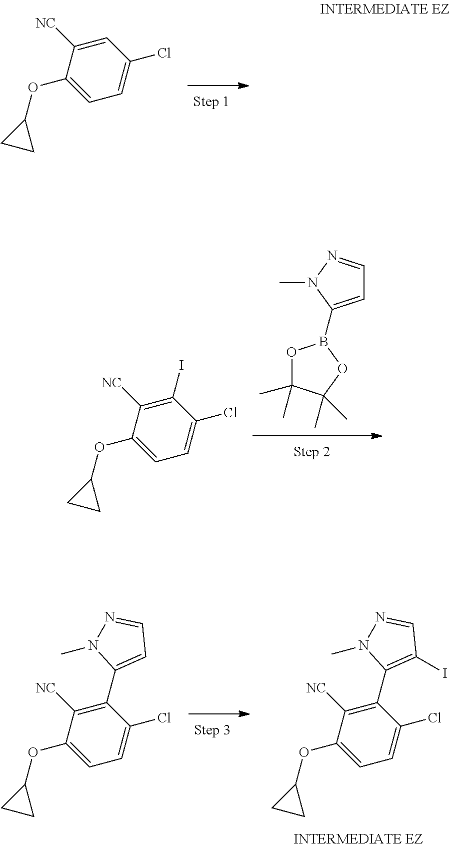

6-cyclopropoxy-3-chloro-2-(4-iodo-1-methyl-1H-pyrazol-5-yl)benzonitrile, intermediate EZ, was prepared as a yellow solid (160 mg, 0.40 mmol, 22;% over 3 steps) following the procedure described for the preparation of Intermediate DQ. LCMS [M+1]⁺=399.9, ¹H NMR (400 MHz, CDCl₃) δ=7.70 (d, J=9.2 Hz, 1H), 7.64 (s, 1H), 7.47 (d, J=9.2 Hz, 1H), 3.92 (td, J=2.8, 5.6 Hz, 1H), 3.78 (s, 3H), 0.99-0.90 (m, 4H).

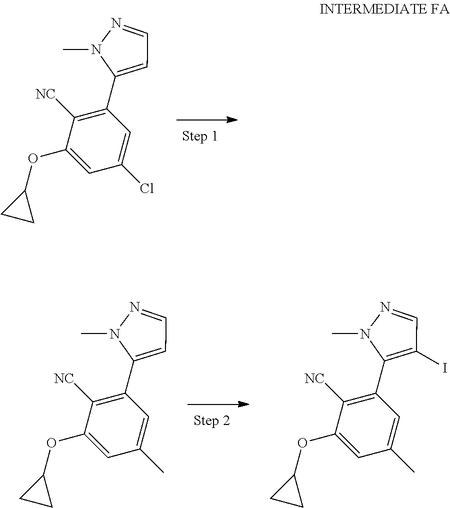 [SMILES: Cn1nccc1-c1cc(Cl)cc(OC2CC2)c1C#N ; Cc1cc(OC2CC2)c(C#N)c(-c2ccnn2C)c1 ; Cc1cc(OC2CC2)c(C#N)c(-c2c(I)cnn2C)c1]

Step 1: To a solution of 4-chloro-2-cyclopropoxy-6-(1-methyl-1H-pyrazol-5-yl)benzonitrile (177 mg, 0.65 mmol, 1.00 eq.) in dioxane (10 mL) was added potassium carbonate (268 mg, 1.94 mmol, 3.00 eq.), di-tert-butyl(cyclopentyl)phosphane;dichloropalladium-iron (42 mg, 0.064 mmol, 0.10 eq.) and methylboronic acid (194 mg, 3.23 mmol, 5.00 eq.). The mixture was stirred at 100° C. for 2 hours then diluted with water (50 mL) and extracted with ethyl acetate (50 mL×3). The combined organic layers were washed with brine (20 mL), dried over anhydrous sodium sulfate, filtered, concentrated and the residue was purified by prep-TLC (SiO₂, petroleum ether:ethyl acetate 25%) to give 2-(cyclopropoxy)-4-methyl-6-(2-methylpyrazol-3-yl)benzonitrile (111 mg, 0.44 mmol, 68% yield) as a white solid. LCMS [M+1]⁺=254.1; ¹H NMR (400 MHz, CDCl₃) δ=7.98 (d, J=2.0 Hz, 1H), 7.65 (d, J=0.8 Hz, 1H), 7.27 (s, 1H), 6.85 (d, J=2.0 Hz, 1H), 4.35-4.29 (m, 1H), 4.27 (s, 3H), 2.91 (s, 3H), 1.36-1.31 (m, 4H).

Step 2: To a solution of 2-(cyclopropoxy)-4-methyl-6-(2-methylpyrazol-3-yl)benzonitrile (100 mg, 0.40 mmol, 1.00 eq.) in acetic acid (2 mL) was added N-iodosuccinimide (178 mg, 0.79 mmol, 2.00 eq.). The mixture was stirred at 25° C. for 1 hour then diluted with water (20 mL) and extracted with ethyl acetate (20 mL×3). The combined organic layers were washed with brine (20 mL), dried over anhydrous sodium sulfate, filtered, concentrated and the residue purified by prep-TLC (SiO₂, petroleum ether:ethyl acetate 25%) to give 2-(cyclopropoxy)-6-(4-iodo-2-methyl-pyrazol-3-yl)-4-methyl-benzonitrile (94 mg, 0.25 mmol, 63% yield) as a yellow solid. LCMS [M+1]⁺=380.0; ¹H NMR (400 MHz, CDCl₃) δ=7.66-7.55 (m, 1H), 7.27 (s, 1H), 6.80 (s, 1H), 3.89 (tt, J=3.2, 6.0 Hz, 1H), 3.85-3.79 (m, 3H), 2.50 (s, 3H), 1.04-0.80 (m, 4H).

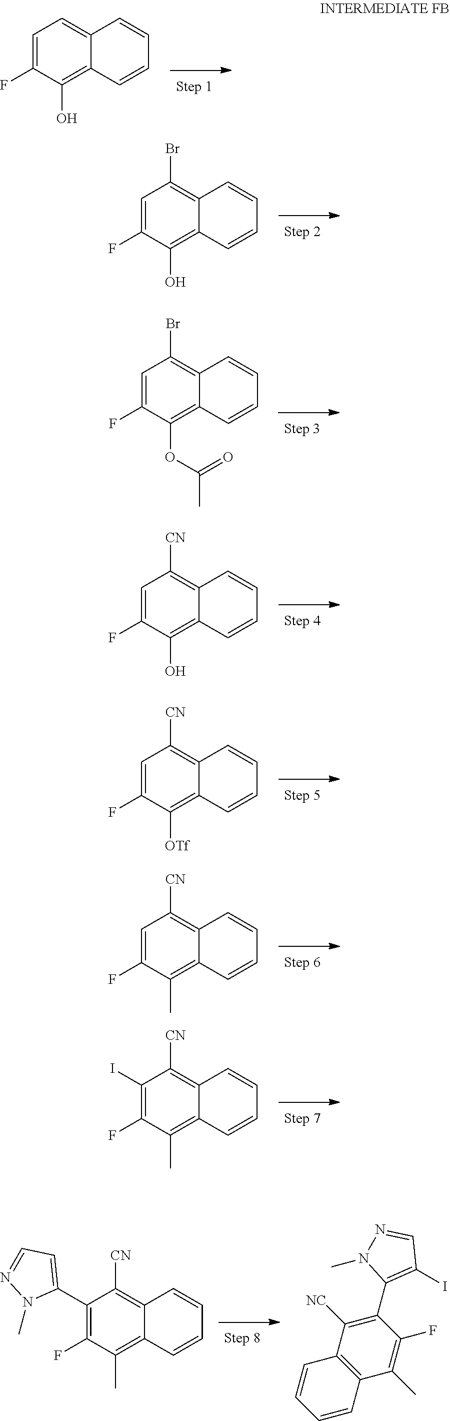

Step 1: To a solution of 2-fluoronaphthalen-1-ol (0.50 g, 3.08 mmol, 1.00 eq.) in dichloromethane (8 mL) was added NBS (521 mg, 2.93 mmol, 0.95 eq.) and the mixture was stirred at −50° C. for 0.25 hr. Water (10 mL) was then added and the separated organic phase was dried, concentrated and the residue purified by column chromatography (SiO₂, petroleum ether/ethyl acetate 1%) to give 4-bromo-2-fluoro-naphthalen-1-ol (500 mg, 2.07 mmol, 67% yield) as a light yellow solid. ¹H NMR (400 MHz, CDCl₃) δ=8.27-8.21 (m, 1H), 8.19-8.11 (m, 1H), 7.63 (d, J=10.2 Hz, 1H), 7.61-7.54 (m, 2H), 5.68 (br d, J=4.0 Hz, 1H).

Step 2: To a solution of 4-bromo-2-fluoro-naphthalen-1-ol (2.30 g, 9.54 mmol, 1.00 eq.), DIEA (21.0 mmol, 3.66 mL, 2.20 eq.) and DMAP (58 mg, 0.48 mmol, 0.05 eq.) in dichloromethane (40 mL) at 0° C. was added acetyl chloride (19.1 mmol, 1.36 mL, 2.00 eq.) in a dropwise fashion. The mixture was stirred at 28° C. for 1 hr before the mixture was concentrated and the formed residue purified by column chromatography (SiO₂, pether/ethyl acetate 1%) to give (4-bromo-2-fluoro-1-naphthyl) acetate (2.50 g, 8.83 mmol, 93% yield) as a white solid. ¹H NMR (400 MHz, CDCl₃) δ=8.26-8.18 (m, 1H), 7.94-7.86 (m, 1H), 7.72 (d, J=9.2 Hz, 1H), 7.66-7.56 (m, 2H), 2.51 (s, 3H).

Step 3: A mixture of (4-bromo-2-fluoro-1-naphthyl) acetate (2.50 g, 8.83 mmol, 1.00 eq.), Pd₂(dba)₃ (809 mg, 0.88 mmol, 0.10 eq.), Zn(CN)₂ (9.71 mmol, 0.62 mL, 1.10 eq.), Zn (29 mg, 0.442 mmol, 0.05 eq.) and DPPF (979 mg, 1.77 mmol, 0.20 eq.) in DMA (40 mL) was degassed with nitrogen 3 times then stirred at 120° C. for 3 hr. The reaction mixture was then diluted with ethyl acetate (100 mL), filtered and the filtrate was washed with brine (50 mL×3), dried, concentrated and the residue was purified by column chromatography (SiO₂, petroleum ether/ethyl acetate 2-100%) to give 3-fluoro-4-hydroxy-naphthalene-1-carbonitrile (980 mg, 5.24 mmol, 59/6 yield) as a yellow solid. ¹H NMR (400 MHz, CDCl₃) δ=8.36-8.27 (m, 1H), 8.18 (d, J=8.0 Hz, 1H), 7.76 (d, J=9.6 Hz, 1H), 7.72-7.62 (m, 2H).

Step 4: To a solution of 3-fluoro-4-hydroxy-naphthalene-1-carbonitrile (880 mg, 4.70 mmol, 1.00 eq.) in dichloromethane (20 mL) was added triethylamine (9.40 mmol, 1.31 mL, 2.00 eq.) and Tf₂O (8.46 mmol, 1.40 mL, 1.80 eq.) at 0° C. The mixture was then stirred at 28° C. for 0.5 hr. The reaction mixture was then concentrated, and the residue purified by column chromatography (SiO₂, petroleum ether/ethyl acetate 2%) to give (4-cyano-2-fluoro-1-naphthyl) trifluoromethanesulfonate (1.15 g, 3.60 mmol, 77% yield) as a yellow solid.

Step 5: A mixture of (4-cyano-2-fluoro-1-naphthyl) trifluoromethanesulfonate (0.38 g, 1.19 mmol, 1.00 eq.), Pd(PPh₃)₄ (138 mg, 0.12 mmol, 0.10 eq.), AlMe₃ (2 M in PhMe, 1.79 mL, 3.00 eq.) in toluene (3.5 mL) was degassed with nitrogen 3 times and stirred at 120° C. for 2 hr. After such time the reaction was quenched by the addition of water (5 mL) then diluted with ethyl acetate (40 ml) and filtered. The filtrate was washed with water (20 mL×3), dried, concentrated and the residue purified by prep-TLC (SiO₂, Petroleum ether/Ethyl acetate 5%) to give compound 3-fluoro-4-methyl-naphthalene-1-carbonitrile (90 mg, 0.49 μmol, 41% yield) as a white solid. ¹H NMR (400 MHz, CDCl₃) δ=8.29-8.22 (m, 1H), 8.11-8.03 (m, 1H), 7.75-7.63 (m, 3H), 2.66 (d, J=2.4 Hz, 3H).

Step 6: To a solution of N-isopropylpropan-2-amine (0.86 mmol, 0.12 mL, 2.00 eq.) in THF (2 mL) was added n-BuLi (2.5 M in THF, 0.31 mL, 1.80 eq.) in a dropwise fashion at −70° C. The mixture was then stirred at −70° C. for 0.5 hr, then 3-fluoro-4-methyl-naphthalene-1-carbonitrile (80 mg, 0.43 mmol, 1 eq.) was added and stirred for a further 0.5 hr before iodine (0.87 mmol, 0.17 mL, 2.00 eq.) was added. After completion of the addition the mixture was stirred at 30° C. for 1 hr. The reaction mixture was then quenched by the addition of water (2 mL) and extracted with ethyl acetate (10 mL×2). The combined organic phases were dried and concentrated to give 3-fluoro-2-iodo-4-methyl-naphthalene-1-carbonitrile (110 mg, 0.35 mmol) as a brown solid.

Step 7: A mixture of 3-fluoro-2-iodo-4-methyl-naphthalene-1-carbonitrile (110 mg, 0.35 mmol, 1.00 eq.), 1-methyl-5-(4,4,5,5-tetramethyl-1,3,2-dioxaborolan-2-yl)pyrazole (96 mg, 0.46 mmol, 1.30 eq.), [2-(2-aminophenyl)phenyl]palladium(1+)-bis(1-adamantyl)-butyl-phosphane-methanesulfonate (26 mg, 0.035 mmol, 0.10 eq.), K₃PO₄ (1.50 M, 0.71 mL, 3.00 eq.) in n-butyl alcohol (2.8 mL) was degassed with nitrogen 3 times then stirred at 60° C. for 6 hr. The reaction mixture was then filtered, concentrated and the residue was purified by prep-TLC (SiO₂, petroleum ether/ethyl acetate 20%) to give 3-fluoro-4-methyl-2-(2-methylpyrazol-3-yl)naphthalene-1-carbonitrile (50 mg, 0.19 mmol, 53% yield) as a brown solid. LCMS [M+1]⁺ 266.1.

Step 8: To a solution of 3-fluoro-4-methyl-2-(2-methylpyrazol-3-yl)naphthalene-1-carbonitrile (50 mg, 0.19 mmol, 1.00 eq.) in acetic acid (2 mL) was added NIS (127 mg, 0.57 mmol, 3.00 eq.). The mixture was stirred at 30° C. for 12 hrs. The pH was then adjusted to pH 7 with saturated sodium bicarbonate aqueous solution and then extracted with ethyl acetate (10 mL×3) and the combined organic phases were dried and concentrated. The residue was purified by prep-TLC (SiO₂, petroleum ether/ethyl acetate 20%) to give compound 3-fluoro-2-(4-iodo-2-methyl-pyrazol-3-yl)-4-methyl-naphthalene-1-carbonitrile (30 mg, 0.076 mmol, 41% yield) as a brown solid. LCMS [M+1]⁺ 392.0.

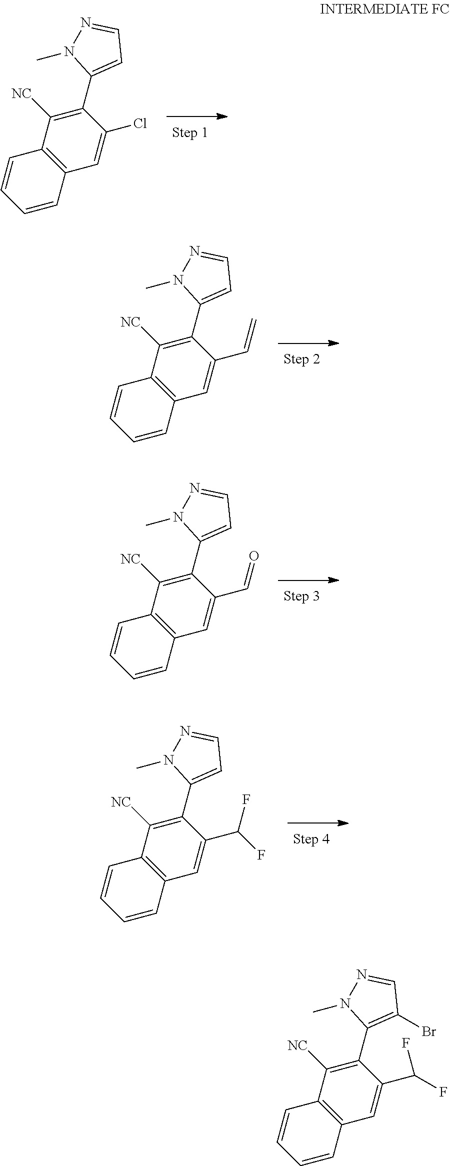 [SMILES: Cn1nccc1-c1c(Cl)cc2ccccc2c1C#N ; C=Cc1cc2ccccc2c(C#N)c1-c1ccnn1C ; Cn1nccc1-c1c(C=O)cc2ccccc2c1C#N ; Cn1nccc1-c1c(C(F)F)cc2ccccc2c1C#N ; Cn1ncc(Br)c1-c1c(C(F)F)cc2ccccc2c1C#N]

Step 1: To a solution of 3-chloro-2-(1-methyl-1H-pyrazol-5-yl)-1-naphthonitrile (200 mg, 0.747 mmol, 1.00 eq), potassium trifluoro(vinyl)borate (120 mg, 0.896 mmol, 1.20 eq), cesium carbonate (730 mg, 2.24 mmol, 3.00 eq) and dicyclohexyl(2′,6′-diisopropoxy-[1,1′-biphenyl]-2-yl)phosphine (34.9 mg, 0.074 mmol, 0.10 eq) in THF (2 mL) and water (0.2 mL) was added palladium(II) chloride (13.3 mg, 0.074 mmol, 0.10 eq) under a nitrogen atmosphere. The resulting mixture was stirred at 90° C. for 16 hours. The mixture was then diluted with water (3 mL) and extracted with ethyl acetate (5 mL×3). The combined organic layers were washed with brine (5 mL×2), dried over anhydrous sodium sulfate, filtered and concentrated under reduced pressure. The residue was purified by column chromatography (SiO₂, Petroleum ether/Ethyl acetate 5-20%) to give 2-(1-methyl-1H-pyrazol-5-yl)-3-vinyl-1-naphthonitrile (130 mg, 0.049 mmol, 66% yield) as a light yellow solid. LCMS [M+1]⁺=260.2.

Step 2: 2-(1-methyl-1H-pyrazol-5-yl)-3-vinyl-1-naphthonitrile (130 mg, 491 μmol, 1.00 eq) were dissolved in dichloromethane (20 mL) and cooled to −70° C. Ozone was bubbled into the reaction solution with stirring for 15 min. Dimethylsulfane (8.46 g, 136 mmol, 10 mL, 277 eq) was then added and the mixture stirred at −70° C. for 15 min. After such time the mixture was concentrated and then purified by column chromatography (SiO₂, Petroleum ether/Ethyl acetate 5-20%) to give 3-formyl-2-(1-methyl-1H-pyrazol-5-yl)-1-naphthonitrile (60 mg, 222 μmol, 45.3% yield) as a white solid. LCMS [M+1]⁺=262.2.

Step 3: A solution of 3-formyl-2-(1-methyl-1H-pyrazol-5-yl)-1-naphthonitrile (20 mg, 74.2 μmol, 1.00 eq) and (bis-(2-methoxyethyl)amino)sulfur trufluoride (41 mg, 185 μmol, 41 μL, 2.50 eq) in dichloromethane (1.0 mL) was stirred at 25° C. for 6 hours. The reaction was then quenched with saturated sodium bicarbonate aqueous (2 mL) and extracted with ethyl acetate (5 mL×3). The combined organic layers were washed by brine (3 mL×3), dried over anhydrous sodium sulfate, filtered, concentrated and the residue purified by Prep-TLC (Petroleum ether/Ethyl acetate 20%) to give 3-(difluoromethyl)-2-(2-methylpyrazol-3-yl)naphthalene-1-carbonitrile (11.0 mg, 38.4 μmol, 52% yield) as a white solid. LCMS [M+1]⁺=284.2.

Step 4: To a solution of 3-(difluoromethyl)-2-(1-methyl-1H-pyrazol-5-yl)-1-naphthonitrile (11 mg, 38 μmol, 1.00 eq) in dichloromethane (1.0 mL) was added 1-bromopyrrolidine-2,5-dione (10 mg, 58 μmol, 1.50 eq). The mixture was stirred at 25° C. for 10 hours. The reaction mixture was the concentrated and purified by Prep-TLC (SiO₂, Petroleum ether/Ethyl acetate 20%) to give 2-(4-bromo-1-methyl-1H-pyrazol-5-yl)-3-(difluoromethyl)-1-naphthonitrile (12 mg, 33 μmol, 86% yield) as a colorless oil. LCMS [M+1]⁺=362.1.

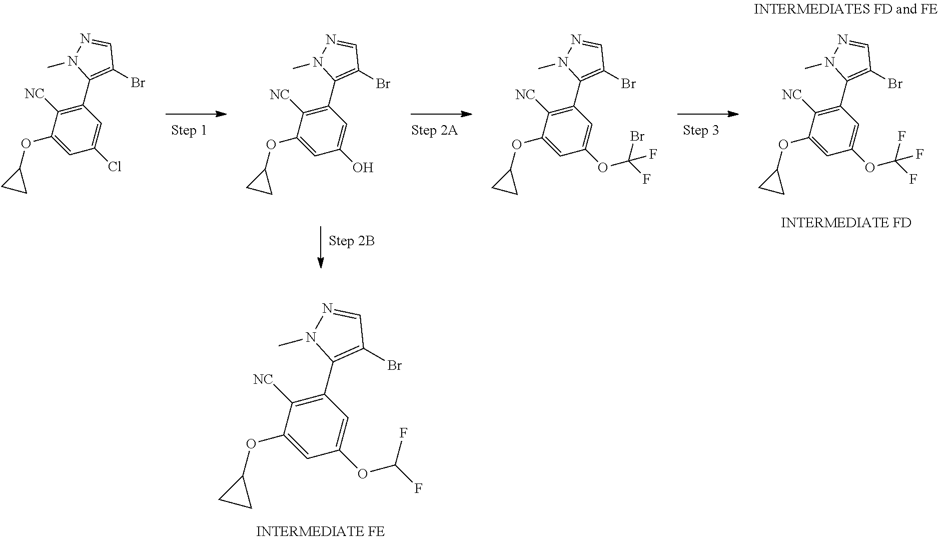 [SMILES: Cn1ncc(Br)c1-c1cc(Cl)cc(OC2CC2)c1C#N ; Cn1ncc(Br)c1-c1cc(O)cc(OC2CC2)c1C#N ; Cn1ncc(Br)c1-c1cc(OC(F)(F)Br)cc(OC2CC2)c1C#N ; Cn1ncc(Br)c1-c1cc(OC(F)(F)F)cc(OC2CC2)c1C#N ; Cn1ncc(Br)c1-c1cc(OC(F)F)cc(OC2CC2)c1C#N]

Step 1: A mixture of sodium hidride (51 mg, 1.28 mmol, 60% purity, 1.20 eq.) and 2-methylsulfonylethanol (158 mg, 1.28 mmol, 1.20 eq.) in DMF (3 mL) at 0° C. under nitrogen was stirred at 0° C. for 30 minutes. Then, a solution of 2-(4-bromo-2-methyl-pyrazol-3-yl)-4-chloro-6-(cyclopropoxy)benzonitrile (375 mg, 1.06 mmol, 1 eq.) in DMF (2 mL) was added dropwise and the reaction mixture was stirred at 25° C. for 2 hours. The mixture was then quenched with water (5 mL) and extracted with ethyl acetate (3×10 mL). The combined organic layers were dried over sodium sulfate, filtered, concentrated and purified by column chromatography (SiO₂, Petroleum ether/Ethyl acetate 10-30%) to give 2-(4-bromo-2-methyl-pyrazol-3-yl)-6-(cyclopropoxy)-4-hydroxy-benzonitrile (120 mg, 33% yield) as colorless oil. LCMS [M+1]⁺=334.0; ¹H NMR (400 MHz, CDCl₃) δ=9.77-9.37 (m, 1H), 7.54 (s, 1H), 6.98 (d, J=2.0 Hz, 1H), 6.51 (d, J=2.0 Hz, 1H), 3.85-3.82 (m, 1H), 3.80 (s, 3H), 0.94-0.84 (m, 5H).

Step 2A: To a mixture of 2-(4-bromo-2-methyl-pyrazol-3-yl)-6-(cyclopropoxy)-4-hydroxy-benzonitrile (20 mg, 59 μmol, 1.00 eq.) in DMF (0.5 mL) was added sodium hydride (4.8 mg, 119 μmol, 60% purity, 2.00 eq.) at 0° C. under nitrogen and the mixture was stirred at 0° C. for 30 minutes. Then dibromo(difluoro)methane (38 mg, 180 μmol, 17 μL, 3.00 eq.) was added to the mixture at 0° C. and the mixture was stirred at 25° C. for 1 hour. Water (3.0 mL) was then added and the mixture extracted with ethyl acetate (3×5 mL). The combined organic layers were dried over sodium sulfate, filtered, concentrated under vacuum and the residue was purified by prep-TLC (SiO₂, petroleum ether:ether acetate 20%) to give 4-[bromo(difluoro)methoxy]-2-(4-bromo-2-methyl-pyrazol-3-yl)-6-(cyclopropoxy)benzonitrile (10.0 mg, 36% yield) as a white solid. LCMS [M+1]⁺=463.9

Step 2B: To a mixture of 2-(4-bromo-2-methyl-pyrazol-3-yl)-6-(cyclopropoxy)-4-hydroxy-benzonitrile (100 mg, 299 μmol, 1.00 eq.) and sodium 2-chloro-2,2-difluoro-acetate (114 mg, 748 μmol, 2.50 eq.) in DMF (1.0 mL) and water (0.1 mL) was added cesium carbonate (146 mg, 449 μmol, 1.50 eq.). The mixture was stirred at 100° C. for 3 hours. The reaction mixture was then quenched with water (5 mL) and extracted with ethyl acetate (3×10 mL). The combined organic phases were washed with brine (15 mL), dried with anhydrous sodium sulfate, filtered, concentrated in vacuum and the residue purified by column chromatography (SiO₂, Petroleum ether/Ethyl acetate 0-30%) to give 2-(4-bromo-2-methyl-pyrazol-3-yl)-6-(cyclopropoxy)-4-(difluoromethoxy)benzonitrile (70 mg, 61% yield) as colorless oil. LCMS [M+1]⁺=386.0; ¹H NMR (400 MHz, CDCl₃) δ=7.57 (s, 1H), 7.23 (d, J=2.4 Hz, 1H), 6.87-6.43 (m, 2H), 3.95-3.87 (m, 1H), 3.82 (s, 3H), 0.99-0.90 (m, 4H)

Step 3: A mixture of 4-[bromo(difluoro)methoxy]-2-(4-bromo-2-methyl-pyrazol-3-yl)-6-(cyclopropoxy)benzonitrile (100 mg, 216 μmol, 1.00 eq.) and silver tetrafluoroborate (273 mg, 1.40 mmol, 6.50 eq.) in DCE (2. mL) was stirred at 65° C. for 3 hours. Then, the reaction mixture was quenched with water (5 mL) and extracted with ethyl acetate (3×10 mL). The combined organic phases were washed with brine (15 mL), dried with anhydrous sodium sulfate, filtered, concentrated in vacuum and the residue purified by reversed phase flash (0.1% FA condition) to give 2-(4-bromo-2-methyl-pyrazol-3-yl)-6-(cyclopropoxy)-4-(trifluoromethoxy)benzonitrile (55 mg, 63% yield) as a yellow solid. LCMS [M+1]⁺=402.1.

In one aspect of the invention, provided herein are Intermediates that may be used in the preparation of compounds of Formula (I), Formula (I-A), Formula (I-B), Formula (I-C), Formula (1-D), Formula HA, Formula IIB, Formula IIC, Formula IVA, Formula IVB, Formula IVC, Formula IIA-1, Formula IIB-1, Formula IIC-1, Formula IVA-1, Formula IVB-1 and/or Formula IVC-1. In one embodiment, the intermediates include Intermediates A-1 through HB.

In one aspect of the invention, provided herein are Intermediates that may be used in the preparation of compounds of Formula IIA, IIB, IIC, IVA, IVB, IVC, IIA-1, IIB-1, IIC-1, IVA-1, IVB-1 and IVC-1.

In one embodiment, the intermediates include Intermediates A-1 through HQ

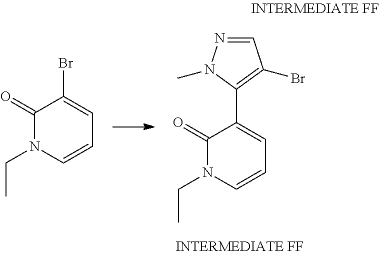

A mixture of 3-bromo-1-ethylpyridin-2 (1H)-one (1.00 g, 4.95 mmol, 1.00 eq.), 4-bromo-1-methyl-1H-pyrazole (797 mg, 4.95 mmol, 1.00 eq.), Pd(OAc)₂ (11.1 mg, 49.5 μmol, 0.01 eq.), DavePhos (39.0 mg, 99.0 μmol, 0.02 eq.), 2-methylpropanoic acid (131 mg, 1.48 mmol, 138 μL, 0.30 eq.) and tetrabutylammonium acetate (2.98 g, 9.90 mmol, 3.01 mL, 2.00 eq.) in N-methyl pyrrolidone (10.0 mL) was degassed and stirred at 100° C. for 12 hours under nitrogen atmosphere. The mixture was poured into water (50 mL). The aqueous phase was extracted with ethyl acetate (20 mL). The combined organic phase was washed with brine (50 mL×2), dried over anhydrous sodium sulfate, filtered, and concentrated under reduced pressure. The residue was purified by column chromatography (SiO₂, Ethyl acetate/Petroleum ether 0-50%) to give 3-(4-bromo-1-methyl-1H-pyrazol-5-yl)-1-ethylpyridin-2 (1H)-one (420 mg, 29% yield) as a white solid. LCMS [M+1]⁺=282.1/284.1. 1H NMR (400 MHz, CDCl₃) δ=7.52 (dd, J=2.0, 7.2 Hz, 1H), 7.51 (s, 1H), 7.46 (dd, J=2.0, 6.8 Hz, 1H), 6.34 (t, J=6.8 Hz, 1H), 4.09 (q, J=7.2 Hz, 2H), 3.84 (s, 3H), 1.43 (t, J=7.2 Hz, 3H).

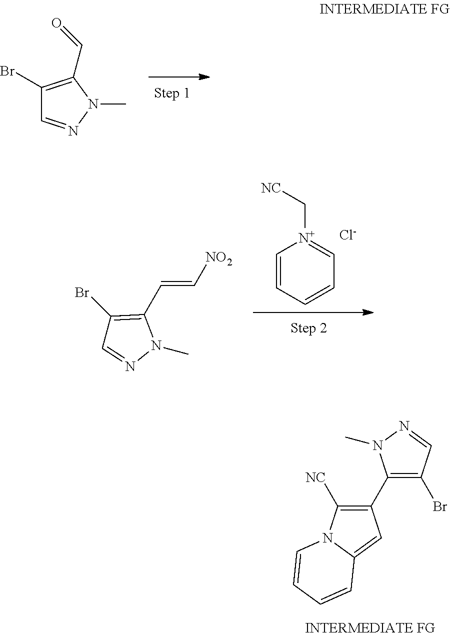

Step 1: To a solution of 4-bromo-2-methyl-pyrazole-3-carbaldehyde (1.00 g, 5.29 mmol, 1.00 eq.) and nitromethane (420 mg, 6.88 mmol, 372 μL, 1.30 eq.) in methyl alcohol (10 mL) was added a solution of sodium hydroxide (466 mg, 11.6 mmol, 2.20 eq.) in water (1.0 mL) drop-wise at 0° C. The reaction mixture was stirred at 0° C. for 0.5 hour. The reaction mixture was quenched by addition hydrochloric acid (1.00 M, 5.0 mL), filtered and concentrated under reduced pressure to give 4-bromo-1-methyl-5-[(E)-2-nitrovinyl]pyrazole (627 mg, crude) as a yellow solid which used into the next step without further purification. ¹H NMR (400 MHz, CDCl₃) δ=8.10 (d, J=13.6 Hz, 1H), 7.93 (d, J=13.6 Hz, 1H), 7.57 (s, 1H), 4.03 (s, 3H)

Step 2: A suspension of 2-pyridin-1-ium-1-ylacetonitrile chloride (627 mg, 4.05 mmol, 1.50 eq.) and 4A MS (1.00 g, 215 μmol) in dichloroethane (30 mL) was cooled to 0° C. using an ice-cooling bath, and then 2,6-lutidine (1.45 g, 13.5 mmol, 1.57 mL, 5.00 eq.) was added. After stirring for 15 minutes, 4-bromo-1-methyl-5-[(E)-2-nitrovinyl]pyrazole (627 mg, 2.70 mmol, 1.00 eq.) was added followed by cupric acetate (736 mg, 4.05 mmol, 1.50 eq.). The mixture was stirred at 0° C. for 15 minutes and then warmed to 25° C. and stirred at 25° C. for 5 hours. The reaction mixture was diluted with water (300 mL) and extracted with EtOAc (100 mL×3). The combined organic layers were dried over anhydrous sodium sulfate, filtered and concentrated under reduced pressure and the residue was purified by column chromatography (SiO₂, Ethyl acetate/Petroleum ether, 10-35%) to give 2-(4-bromo-2-methyl-pyrazol-3-yl)indolizine-3-carbonitrile (380 mg, 47% yield) as a yellow solid. LCMS [M+1]⁺=301.0. ¹H NMR (400 MHz, CDCl₃) δ=8.34 (d, J=6.0 Hz, 1H), 7.62-7.53 (m, 2H), 7.18-7.11 (m, 1H), 6.96 (dt, J=1.2, 6.8 Hz, 1H), 6.62 (s, 1H), 3.91 (s, 3H)

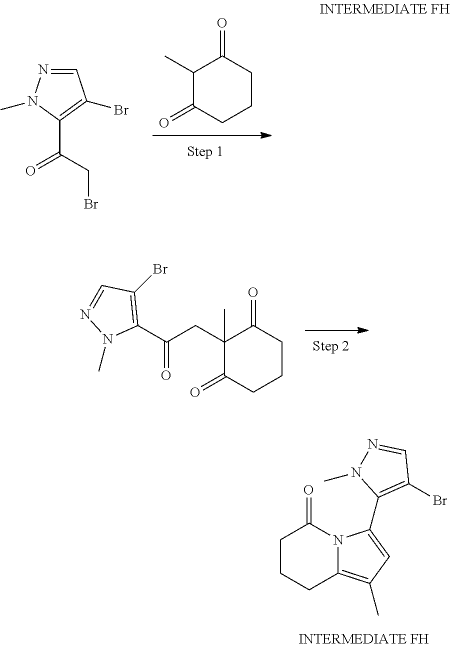

Step 1: To a solution of 2-bromo-1-(4-bromo-2-methyl-pyrazol-3-yl)ethanone (1.60 g, 5.68 mmol, 1.00 eq.) and 2-methylcyclohexane-1,3-dione (859 mg, 6.81 mmol, 1.20 eq.) in dimethyl formamide (26 mL) was added potassium carbonate (1.57 g, 11.4 mmol, 2.00 eq.). The mixture was stirred at 25° C. for 2 hours. The mixture was diluted with water (120 mL) and extracted with ethyl acetate (30 mL×3). The combined organic layers were washed with brine, dried over sodium sulfate, filtered and concentrated. The residue was purified by flash silica gel chromatography (Ethyl acetate/Petroleum ether, 20-25%) to give 2-[2-(4-bromo-2-methyl-pyrazol-3-yl)-2-oxo-ethyl]-2-methyl-cyclohexane-1,3-dione (500 mg, 24% yield) as a white solid. LCMS [M+1]⁺=327.0. ¹H NMR (400 MHz, DMSO-d₆) δ=7.51-7.47 (m, 1H), 4.01 (s, 3H), 3.81 (s, 2H), 2.77 (dd, J=5.6, 7.6 Hz, 4H), 2.37-2.25 (m, 1H), 2.21-2.10 (m, 1H), 1.38 (s, 3H).

Step 2: A mixture of 2-[2-(4-bromo-2-methyl-pyrazol-3-yl)-2-oxo-ethyl]-2-methyl-cyclohexane-1,3-dione (500 mg, 1.53 mmol, 1.00 eq.) and ammonium acetate (2.36 g, 30.6 mmol, 20.0 eq.) in acetic acid (10.0 mL) was stirred at 140° C. for 12 hours. The mixture was adjusted to pH=8-9 with saturated sodium carbonate and extracted with ethyl acetate (20 mL×3). The combined organic layers were washed with brine, dried over sodium sulfate, filtered and concentrated. The residue was purified by flash silica gel chromatography (Ethyl acetate/Petroleum ether, 20-25%) to give 3-(4-bromo-2-methyl-pyrazol-3-yl)-1-methyl-7,8-dihydro-6H-indolizin-5-one (110 mg, 23% yield) as a white solid. LCMS [M+1]⁺=308.0. ¹H NMR (400 MHz, CDCl3-d₆) δ=7.48 (s, 1H), 6.24 (s, 1H), 3.68 (s, 3H), 2.89-2.76 (m, 2H), 2.74-2.63 (m, 2H), 2.15-2.07 (m, 2H), 2.05 (s, 3H).

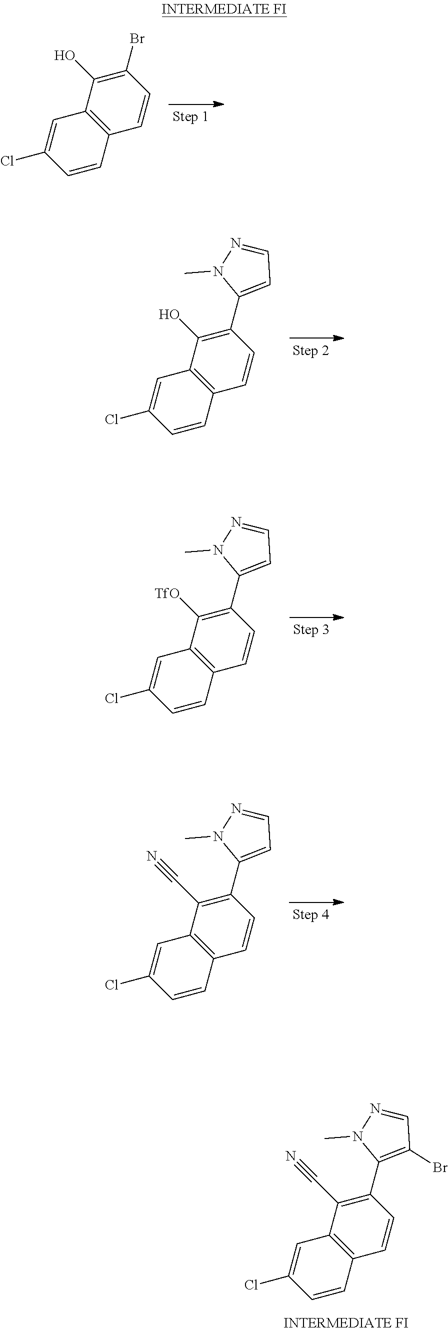 [SMILES: Oc1c(Br)ccc2ccc(Cl)cc12 ; Cn1nccc1-c1ccc2ccc(Cl)cc2c1O ; Cn1nccc1-c1ccc2ccc(Cl)cc2c1OS(=O)(=O)C(F)(F)F ; Cn1nccc1-c1ccc2ccc(Cl)cc2c1C#N ; Cn1ncc(Br)c1-c1ccc2ccc(Cl)cc2c1C#N]

Step 1: A mixture of 2-bromo-7-chloro-naphthalen-1-ol (700 mg, 2.72 mmol, 1.00 eq.), 1-methyl-5-(4,4,5,5-tetramethyl-1,3,2-dioxaborolan-2-yl)pyrazole (679 mg, 3.26 mmol, 1.20 eq.), potassium carbonate (564 mg, 4.08 mmol, 1.50 eq.), Pd(dtbpf)Cl₂ (177 mg, 272 μμmol, 0.10 eq.) in dioxane (5.0 mL) and water (1.0 mL) was degassed and stirred at 80° C. for 2 hours under nitrogen atmosphere. The reaction mixture was concentrated under reduced pressure and the residue was purified by prep-TLC (SiO₂, Petroleum ether/Ethyl acetate 2:1) to give 7-chloro-2-(2-methylpyrazol-3-yl)naphthalen-1-ol (500 mg, 71% yield) as a white solid. LCMS [M+1]⁺=259.0.

Step 2: To a solution of 7-chloro-2-(2-methylpyrazol-3-yl)naphthalen-1-ol (470 mg, 1.82 mmol, 1.00 eq.) and triethylamine (552 mg, 5.45 mmol, 759 μL, 3.00 eq.) in dichloromethane (10.0 mL) was added trifluoromethanesulfonic anhydride (1.03 g, 3.63 mmol, 600 μL, 2.00 eq.) at 0° C. The mixture was stirred at 25° C. for 2 hours. The reaction mixture was concentrated under reduced pressure and the residue was purified by column chromatography (SiO₂, Ethyl acetate/Petroleum ether, 10-25%) to give [7-chloro-2-(2-methylpyrazol-3-yl)-1-naphthyl]trifluoromethanesulfonate (450 mg, 63% yield) as a white solid.

LCMS [M+1]-=390.9. ¹H NMR (400 MHz, CDCl₃) δ=8.17 (d, J=2.0 Hz, 1H), 7.95 (d, J=8.0 Hz, 1H), 7.92 (d, J=8.8 Hz, 1H), 7.65-7.59 (m, 2H), 7.48 (d, J=8.4 Hz, 1H), 6.45 (d, J=1.6 Hz, 1H), 3.82 (s, 3H).

Step 3: A mixture of [7-chloro-2-(2-methylpyrazol-3-yl)-1-naphthyl]trifluoromethanesulfonate (200 mg, 512 μμmol, 1.00 eq.), Pd₂(dba)₃ (46.9 mg, 51.2 μμmol, 0.10 eq.), zinc cyanide (33.1 mg, 282 μmol, 17.9 μL, 0.55 eq.), zinc powder (3.35 mg, 51.2 μmol, 0.10 eq.) and dppf (56.8 mg, 102 μμmol, 0.20 eq.) in dimethyl formamide (5.0 mL) was degassed and stirred at 100° C. for 2 hours under nitrogen atmosphere. The reaction mixture was diluted with water (30 mL) and extracted with ethyl acetate (50 mL×3). The combined organic layers were washed with brine (150 mL), dried over anhydrous sodium sulfate, filtered and concentrated under reduced pressure and the residue was purified by prep-TLC (SiO₂, Petroleum ether/Ethyl acetate 1:1) to give 7-chloro-2-(2-methylpyrazol-3-yl)naphthalene-1-carbonitrile (100 mg, 73% yield) as a white solid. LCMS [M+1]⁺=268.1. ¹H NMR (400 MHz, CDCl₃) δ=8.35 (d, J=2.0 Hz, 1H), 8.14 (d, J=8.4 Hz, 1H), 7.94 (d, J=8.8 Hz, 1H), 7.70-7.61 (m, 2H), 7.54 (d, J=8.4 Hz, 1H), 6.60 (d, J=2.0 Hz, 1H), 3.90 (s, 3H).

Step 4: To a solution of 7-chloro-2-(2-methylpyrazol-3-yl)naphthalene-1-carbonitrile (90 mg, 336 μμmol, 1.00 eq.) in acetonitrile (2.0 mL) was added N-bromo-succinimide (120 mg, 672 μμmol, 2.00 eq.). The mixture was stirred at 27° C. for 1 hour. The reaction mixture was concentrated under reduced pressure and the residue was purified by prep-TLC (SiO₂, Petroleum ether/Ethyl acetate 3:1) to give 2-(4-bromo-2-methyl-pyrazol-3-yl)-7-chloro-naphthalene-1-carbonitrile (90 mg, 77% yield) as a yellow solid. LCMS [M+1]⁺=347.8. ¹H NMR (400 MHz, CDCl₃) δ=8.37 (d, J=2.0 Hz, 1H), 8.19 (d, J=8.4 Hz, 1H), 7.97 (d, J=8.8 Hz, 1H), 7.69 (dd, J=2.0, 8.8 Hz, 1H), 7.65 (s, 1H), 7.52 (d, J=8.4 Hz, 1H), 3.86 (s, 3H).

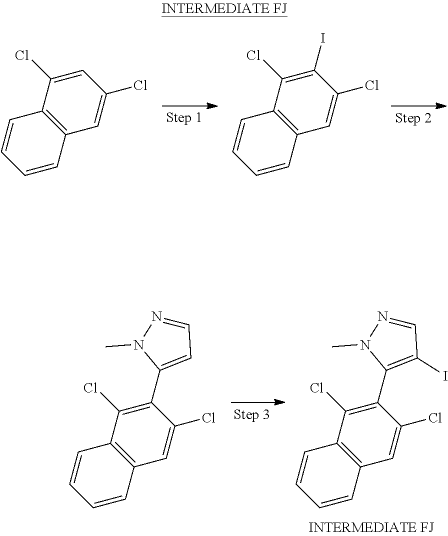 [SMILES: Clc1cc(Cl)c2ccccc2c1 ; Clc1cc2ccccc2c(Cl)c1I ; Cn1nccc1-c1c(Cl)cc2ccccc2c1Cl ; Cn1ncc(I)c1-c1c(Cl)cc2ccccc2c1Cl]

Step 1: A dry 100 mL three-neck round bottom flask was charged with diisopropylamine (1.69 g, 16.8 mmol, 2.37 mL, 1.10 eq.) and THF (20 mL) under nitrogen atmosphere. The temperature was reduced to −70° C., then butyllithium (2.50 M, 6.39 mL, 1.05 eq.) was added drop-wise and maintained the temperature at −70° C. for 30 mins. 1,3-dichloronaphthalene (3.00 g, 15.2 mmol, 1.00 eq.) in THF (5.0 mL) was added to the reaction mixture. After 1 hour, to the reaction mixture was slowly added iodine (4.06 g, 16.0 mmol, 3.22 mL, 1.05 eq.) in THF (5.0 mL). After completion of the addition, the reaction was maintained at −70° C. for 1 hour. The mixture was quenched with saturated sodium sulfite solution (100 mL) and extracted with ethyl acetate (100 mL×3). The combined organic phase was washed with brine (100 mL×3), dried with anhydrous sodium sulfate, filtered and concentrated in vacuum. The residue was purified by prep-TLC (silica gel plate, petroleum ether) to give 1,3-dichloro-2-iodo-naphthalene (450 mg) as a brown oil. ¹H NMR (400 MHz, CDCl₃) δ=8.32-8.21 (m, 1H), 7.99-7.88 (m, 1H), 7.82-7.70 (m, 1H), 7.63-7.52 (m, 2H).

Step 2: A mixture of 1,3-dichloro-2-iodo-naphthalene (450 mg, 1.39 mmol, 1.00 eq.), 1-methyl-5-(4,4,5,5-tetramethyl-1,3,2-dioxaborolan-2-yl)pyrazole (304 mg, 1.46 mmol, 1.05 eq.), Palladium, [2-(amino-κN)[1,1-biphenyl]-2-yl-κC][butylbis(tricyclo[3.3.1.13,7]dec-1-yl)phosphine](methanesulfonato-κO)-(101 mg, 139 μμmol, 0.10 eq.) and ammonium bicarbonate (351 mg, 4.18 mmol, 163 μL, 3.00 eq.) in n-butyl alcohol (10.0 mL) was stirred at 60° C. for 16 hours under nitrogen. The mixture was diluted with water (25 mL) and extracted with ethyl acetate (25 mL×3). The combined organic phase was washed with brine (25 mL 3), dried with anhydrous sodium sulfate, filtered and concentrated in vacuum. The residue was purified by prep-TLC (silica gel plate, petroleum ether/ethyl acetate=5:1) to give 5-(1,3-dichloro-2-naphthyl)-1-methyl-pyrazole (60.0 mg, 15% yield) as a yellow gum. LCMS [M+1]⁺: 277.0 Step 3: A mixture of 5-(1,3-dichloro-2-naphthyl)-1-methyl-pyrazole (60.0 mg, 216 μmol, 1 eq.) and 1-iodopyrrolidine-2,5-dione (244 mg, 1.08 mmol, 5.00 eq.) in N,N-dimethylformamide (3.0 mL) was stirred at 80° C. for 12 hours. The mixture was diluted with water (10 mL) and extracted with ethyl acetate (10 mL×3). The combined organic phase was washed with brine (10 mL×3), dried with anhydrous disodium sulfate, filtered and concentrated in vacuum. The residue was purified by prep-TLC (silica gel plate, petroleum ether/ethyl acetate=10:1) to give 5-(1,3-dichloro-2-naphthyl)-4-iodo-1-methyl-pyrazole (50.0 mg, 57% yield) as a yellow solid. ¹H NMR (400 MHz, CDCl₃) δ=8.39-8.34 (m, 1H), 8.01 (s, 1H), 7.92-7.87 (m, 1H), 7.77-7.65 (m, 3H), 3.77 (s, 3H).

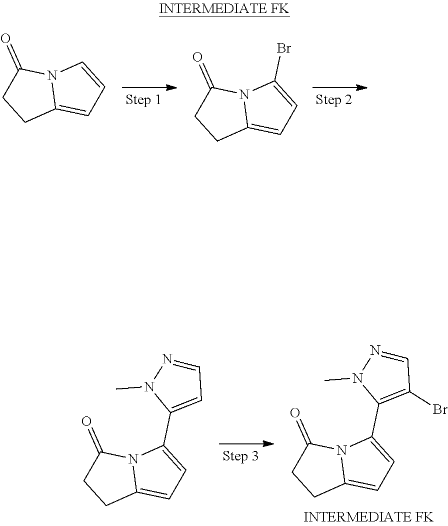 [SMILES: O=C1CCc2cccn21 ; O=C1CCc2ccc(Br)n21 ; Cn1nccc1-c1ccc2n1C(=O)CC2 ; Cn1ncc(Br)c1-c1ccc2n1C(=O)CC2]

Step 1: To a solution of 1,2-dihydropyrrolizin-3-one (200 mg, 1.65 mmol, 1.00 eq.) in THF (8.0 mL) was added N-bromosuccinimide (323 mg, 1.82 mmol, 1.10 eq.). The mixture was stirred at 0° C. for 1 hour. The mixture was concentrated under reduced pressure and the residue was purified by prep-TLC (SiO₂, petroleum ether/ethyl acetate 7:1) to give 5-bromo-1, 2-dihydropyrrolizin-3-one (200 mg, 61% yield) as a white solid. ¹H NMR (400 MHz, CDCl₃) δ=6.31 (d, J=3.2 Hz, 1H), 5.83-5.93 (m, 1H), 2.93-3.04 (m, 2H), 2.82-2.92 (m, 2H)

Step 2: A mixture of 5-bromo-1,2-dihydropyrrolizin-3-one (100 mg, 500 μμmol, 1.00 eq.), 1-methyl-5-(4,4,5,5-tetramethyl-1,3,2-dioxaborolan-2-yl)pyrazole (156 mg, 750 μμmol, 1.50 eq.), Pd(dtbpf)Cl₂ (32.6 mg, 50.0 μμmol, 0.10 eq.), sodium carbonate (106 mg, 1000 μμmol, 2.00 eq.) in dioxane (2.0 mL)/water (0.4 mL) was purged with nitrogen for 3 times and stirred at 80° C. for 1 hour under nitrogen atmosphere. The mixture was concentrated under reduced pressure to give a residue The residue was purified by prep-TLC (SiO₂, petroleum ether/ethyl acetate 3:1) to give 5-(2-methylpyrazol-3-yl)-1,2-dihydropyrrolizin-3-one (70.0 mg, 70% yield) as a yellow solid. LCMS [M+1]⁺=202.1.

Step 3: To a solution of 5-(2-methylpyrazol-3-yl)-1,2-dihydropyrrolizin-3-one (50.0 mg, 248 μμmol, 1.00 eq.) in THF (2.0 mL) was added N-bromosuccinimide (35.4 mg, 200 μmol, 0.80 eq.). The mixture was stirred at 0° C. for 30 minutes. The mixture was concentrated under reduced pressure to give a residue The residue was purified by prep-TLC (SiO₂, petroleum ether/ethyl acetate 2:1) to give 5-(4-bromo-2-methyl-pyrazol-3-yl)-1,2-dihydropyrrolizin-3-one (50.0 mg, 178 μμmol, 72% yield) as a white solid. LCMS [M+1]⁺=280.0. ¹H NMR (400 MHz, CDCl₃) δ=7.52 (s, 1H), 6.61 (d, J=3.2 Hz, 1H), 6.13 (d, J=2.8 Hz, 1H), 3.78 (s, 3H), 3.19-2.97 (m, 4H).

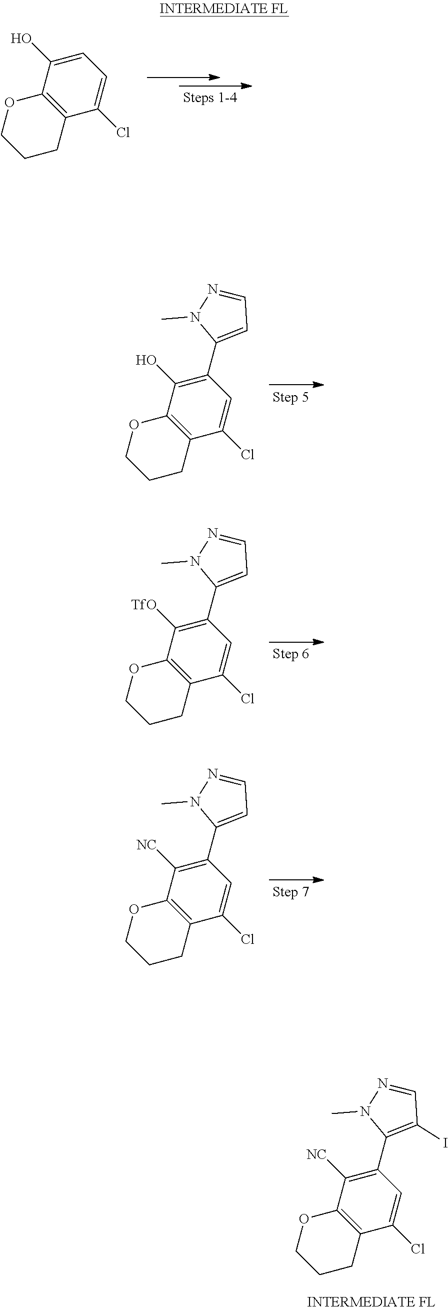 [SMILES: Oc1ccc(Cl)c2c1OCCC2 ; Cn1nccc1-c1cc(Cl)c2c(c1O)OCCC2 ; Cn1nccc1-c1cc(Cl)c2c(c1OS(=O)(=O)C(F)(F)F)OCCC2 ; Cn1nccc1-c1cc(Cl)c2c(c1C#N)OCCC2 ; Cn1ncc(I)c1-c1cc(Cl)c2c(c1C#N)OCCC2]

Step 1: To a solution of 5-chlorochroman-8-ol (100 mg, 542 μμmol, 1.00 eq.) in methanol (1 mL) was added the solution of tetrabutylammonium tribromide (261 mg, 542 μμmol, 1.00 eq.) in dichloromethane (1.5 mL) dropwise under stirring at 25° C. and the reaction mixture was stirred at this temperature for 1 hour. The mixture was concentrated in vacuum. The residue was diluted with ethyl acetate (10 mL) and water (15 mL). The layers were separated and the aqueous phase was extracted with ethyl acetate (3×10 mL). The combined organic layer was dried over sodium sulfate, filtered and concentrated under vacuum to give 7-bromo-5-chloro-chroman-8-ol (160 mg, crude) as a yellow solid. ¹H NMR (400 MHz, CDCl₃)=7.27 (s, 1H), 7.10 (s, 1H), 5.77 (br s, 1H), 4.28-4.23 (m, 2H), 2.73 (t, J=6.8 Hz, 2H), 2.10-2.02 (m, 3H).

Step 2: To a solution of 7-bromo-5-chloro-chroman-8-ol (300 mg, 1.14 mmol, 1.00 eq.) in 2-methylTHF (10.0 mL) was added sodium hydride (91 mg, 2.28 mmol, 60% in mineral oil, 2.00 eq.) at 0° C. After stirring at this temperature for 20 min, chloro(methoxy)methane (458 mg, 5.69 mmol, 432 μL, 5.00 eq.) was added dropwise at 0° C., then the resulting mixture was warmed to 25° C. and stirred for 12 hours. The reaction was quenched by addition of aq. saturated ammonium chloride (20 mL), and washed with aq. saturated sodium hypochlorite (20 mL), diluted with ethyl acetate, then extracted with ethyl acetate (20 mL×3), the combined organic phase was washed with brine (15 mL×3), dried over sodium sulfate, concentrated to deliver the residue. The residue was purified by flash silica gel chromatography (Ethyl acetate/Petroleum ether 1:5) to give 7-bromo-5-chloro-8-(methoxymethoxy)chromane (200 mg, 57% yield) as a colorless oil. ¹H NMR (400 MHz, CDCl₃) □=7.15 (s, 1H), 5.14 (s, 2H), 4.22-4.19 (m, 2H), 3.66 (s, 3H), 2.73 (t, J=6.8 Hz, 2H), 2.07-2.01 (m, 2H).

Step 3: A mixture of 7-bromo-5-chloro-8-(methoxymethoxy)chromane (1.30 g, 4.23 mmol, 1.00 eq.), 1-methyl-5-(4,4,5,5-tetramethyl-1,3,2-dioxaborolan-2-yl)pyrazole (879 mg, 4.23 mmol, 1.00 eq.), sodium bicarbonate (710 mg, 8.45 mmol, 329 μL, 2.00 eq.) and [2-(2-aminophenyl)phenyl]palladium(I)bis(1-adamantyl)-butyl-phosphane methanesulfonate (308 mg, 423 μμmol, 0.10 eq.) in dioxane (100 mL) and water (20 mL) was purged with nitrogen for 3 times and stirred at 80° C. for 20 hours under nitrogen atmosphere. Then the reaction mixture was diluted with water (60 mL), washed with dichloromethane (50 mL×3), brine (50 mL), dried over sodium sulfate, filtered and concentrated to give the crude product. The residue was purified by flash silica gel chromatography (Ethyl acetate/Petroleum ether 0-20%) to give 5-[5-chloro-8-(methoxymethoxy) chroman-7-yl]-1-methyl-pyrazole (500 mg, 38% yield) as white solid.

¹H NMR (400 MHz, CHLOROFORM-d) □=7.50 (t, J=1.6 Hz, 1H), 6.90 (s, 1H), 6.28 (dd, J=2.0, 3.6 Hz, 1H), 4.89 (d, J=9.2 Hz, 2H), 4.27 (td, J=5.2, 10.6 Hz, 2H), 3.80 (s, 3H), 2.99 (d, J=5.2 Hz, 3H), 2.84 (q, J=6.8 Hz, 2H), 2.13-2.05 (m, 2H).

Step 4: To a solution of 5-[5-chloro-8-(methoxymethoxy) chroman-7-yl]-1-methy I-pyrazole (450 mg, 1.46 mmol, 1 eq.), was added trifluoroacetic acid (1.66 g, 14.6 mmol, 1.08 mL, 10.0 eq.) in dichloromethane (5.0 mL). The mixture was stirred at 25° C. for 1 hour. Then the reaction mixture was diluted with dichloromethane (50 mL×3), washed with saturated aqueous sodium bicarbonate (50 mL×3), brine (60 mL), combined organic layers were dried over sodium sulfate, filtered and concentrated under vacuum and the residue was purified by flash silica gel chromatography (Ethyl acetate/Petroleum ether 0-35%) to give 5-chloro-7-(2-methylpyrazol-3-yl) chroman-8-ol (250 mg, 64% yield) as a yellow solid. LC-MS [M+1]⁺=265.1. ¹H NMR (400 MHz, CHLOROFORM-d) δ=7.53-7.52 (m, 1H), 6.74-6.65 (m, 1H), 6.27 (d, J=2.0 Hz, 1H), 4.32-4.27 (m, 2H), 3.81 (d, J=1.6 Hz, 3H), 2.83 (d, J=4.0 Hz, 2H), 2.12-2.06 (m, 2H).

Step 5: To a solution of 5-chloro-7-(2-methylpyrazol-3-yl)chroman-8-ol (250 mg, 944 μmol, 1.00 eq.), triethylamine (287 mg, 2.83 mmol, 394 μL, 3.00 eq.) in dichloromethane (5.0 mL) was stirred at 25° C. for 20 min before being cooled to 0° C. Trifluoromethanesulfonic anhydride (1.07 g, 3.78 mmol, 623 μL, 4.00 eq.) was added and the reaction mixture was then heated to 25° C. and stirred for 19 hours. Then the reaction was quenched with water (20 mL). The mixture was extracted with dichloromethane (15 mL×3), the combined organic extracts were washed brine (10 mL×3) and then dried over anhydrous sodium sulfate, filtered and concentrated under vacuum and the residue was purified by flash silica gel chromatography (Ethyl acetate/Petroleum ether 0-30%) to give [5-chloro-7-(2-methylpyrazol-3-yl) chroman-8-yl]trifluoromethanesulfonate (156 mg, 42% yield) as a yellow oil. LC-MS [M+1]⁺=397.1. ¹H NMR (400 MHz, CHLOROFORM-d) δ=7.54 (d, J=2.0 Hz, 1H), 6.98 (s, 1H), 6.33 (d, J=2.0 Hz, 1H), 4.35-4.30 (m, 2H), 3.78 (s, 3H), 2.88 (t, J=6.8 Hz, 2H), 2.18-2.11 (m, 2H).

Step 6: A degassed solution of [5-chloro-7-(2-methylpyrazol-3-yl)chroman-8-yl]trifluoromethanesulfonate (140 mg, 353 μμmol, 1.00 eq.), zinc cyanide (24.9 mg, 212 μμmol, 13.4 μL, 0.60 eq.), tris(dibenzylideneacetone)dipalladium(0) (64.6 mg, 70.6 μμmol, 0.20 eq.), 1,1′-Bis(diphenylphosphino)ferrocene (19.6 mg, 35.3 μμmol, 0.10 eq.) and zinc powder (4.61 mg, 70.6 μμmol, 0.20 eq.) in N,N-dimethylformamide (2.0 mL) was stirred at 120° C. under nitrogen atmosphere for 4 hours. The mixture was filtered and the filtrate was diluted with ethyl acetate (10 mL) and water (20 mL), the aqueous phase was extracted with ethyl acetate (5.0 mL×3), the organic phase was washed with brine (10 mL×3), dried over sodium sulfate, filtered and concentrated to give the residue. The residue was purified by pre-TLC (Petroleum ether/Ethyl acetate 2:1) to give the 5-chloro-7-(2-methylpyrazol-3-yl)chromane-8-carbonitrile (25.0 mg, 25% yield) as a white solid. LC-MS [M+1]⁺=274.2; [M+23]⁺=296.1.

Step 7: A mixture of 5-chloro-7-(2-methylpyrazol-3-yl)chromane-8-carbonitrile (25.0 mg, 91.3 μmol, 1.00 eq.), NIS (20.6 mg, 91.3 μμmol, 1.00 eq.) in acetonitrile (1.0 mL) was stirred at 40° C. for 2 hour, then heated to 50° C. and stirred for 15 hours. Following that a new portion of NIS (82.2 mg, 365 μμmol, 4.00 eq.) was added to the mixture, then heated to 80° C. and stirred for 8 hours at the same temperature. The reaction was concentrated directly to give the residue, the residue was purified by column chromatography (SiO₂, Petroleum ether/Ethyl acetate 5:1) to give the 5-chloro-7-(4-iodo-2-methyl-pyrazol-3-yl)chromane-8-carbonitrile (21.0 mg, 58% yield) as white solid. LC-MS [M+1]⁺=400.1.

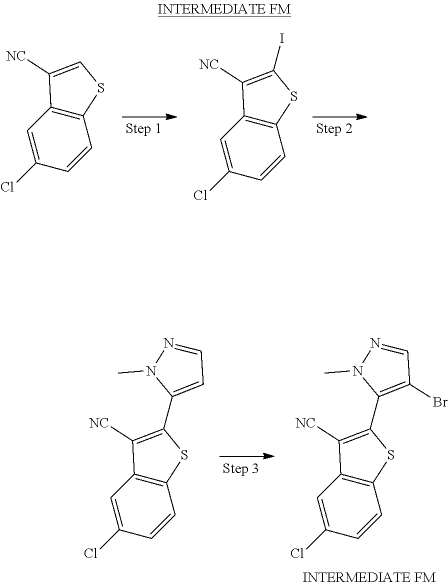 [SMILES: N#Cc1csc2ccc(Cl)cc12 ; N#Cc1c(I)sc2ccc(Cl)cc12 ; Cn1nccc1-c1sc2ccc(Cl)cc2c1C#N ; Cn1ncc(Br)c1-c1sc2ccc(Cl)cc2c1C#N]

Step 1: To a solution of 5-chlorobenzothiophene-3-carbonitrile (200 mg, 1.03 mmol, 1.00 eq.) in THF (10 mL) was added LDA (2.00 M, 775 μL, 1.50 eq.) at −65° C. drop wise and the mixture was stirred for 0.5 hour under nitrogen atmosphere. Following that iodine (524 mg, 2.07 mmol, 416 μL, 2.00 eq.) in THF (10.0 mL) was added drop wise, and then the mixture was warmed up to 25° C. for 2 hours under nitrogen atmosphere. The mixture was quenched with water (1.0 mL) and concentrated under reduced pressure and the residue was purified by column chromatography (SiO₂, Petroleum ether/Ethyl acetate 0-30%) to give 5-chloro-2-iodo-benzothiophene-3-carbonitrile (270 mg, 82% yield) as a white solid. 1H NMR (400 MHz, CDCl₃-d) δ=7.92 (d, J=2.0 Hz, 1H), 7.74 (d, J=8.8 Hz, 1H), 7.42 (dd, J=2.0, 8.8 Hz, 1H).

Step 2: A mixture of 5-chloro-2-iodo-benzothiophene-3-carbonitrile (245 mg, 767 μμmol, 1.00 eq.), 1-methyl-5-(4,4,5,5-tetramethyl-1,3,2-dioxaborolan-2-yl)pyrazole (191 mg, 920 μμmol, 1.20 eq.), sodium bicarbonate (193 mg, 2.30 mmol, 89.5 μL, 3.00 eq.) and Pd(dtbpf)Cl₂ (50.0 mg, 76.7 μmol, 0.10 eq.) in dioxane (5.0 mL) and water (1.0 mL) was purged with nitrogen for 3 times and stirred at 80° C. for 2 hours under nitrogen atmosphere. The mixture was concentrated under reduced pressure and the residue was triturated with Petroleum ether/Ethyl acetate 10:1 (5.0 mL) to give a 5-chloro-2-(2-methylpyrazol-3-yl)benzothiophene-3-carbonitrile (130 mg, 43% yield) as a yellow solid. LCMS [M+1]⁺=273.9.

Step 3: A mixture of 5-chloro-2-(2-methylpyrazol-3-yl)benzothiophene-3-carbonitrile (130 mg, 475 μμmol, 1.00 eq), N-bromosuccinimide (84.5 mg, 475 μμmol, 1.00 eq.) in acetonitrile (3.0 mL) was degassed and stirred at 25° C. for 16 hours under nitrogen atmosphere. The mixture was concentrated under reduced pressure and the residue was triturated with Petroleum ether/Ethyl acetate 2:1 (2.0 mL) then filtered and the filtrate was concentrated under reduced pressure to give 2-(4-bromo-2-methyl-pyrazol-3-yl)-5-chloro-benzothiophene-3-carbonitrile (100 mg, 44% yield, 74% purity) as a yellow solid. LCMS [M+1]⁺=353.9. 1H NMR (400 MHz, CDCl₃-d) δ=8.06 (br d, J=1.6 Hz, 1H), 7.87 (d, J=8.8 Hz, 1H), 7.65 (s, 1H), 7.55 (dd, J=1.6, 8.8 Hz, 1H), 3.98 (s, 3H).

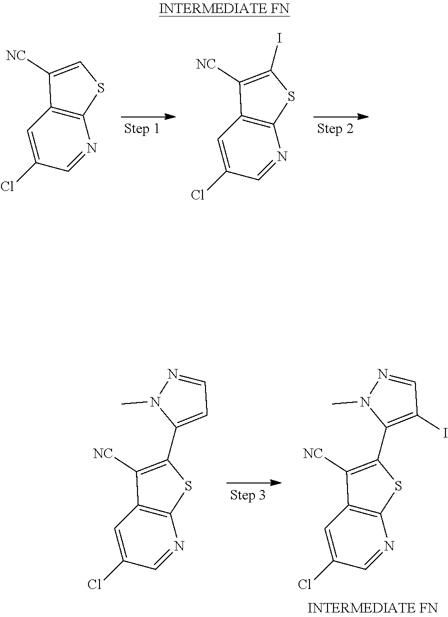 [SMILES: N#Cc1csc2ncc(Cl)cc12 ; N#Cc1c(I)sc2ncc(Cl)cc12 ; Cn1nccc1-c1sc2ncc(Cl)cc2c1C#N ; Cn1ncc(I)c1-c1sc2ncc(Cl)cc2c1C#N]

Step 1: To a solution of diisopropylethylamine (225 mg, 2.22 mmol, 314 μL, 1.60 eq.) in THF (15 mL) was added and n-butyllithium (2.50 M, 832 μL, 1.50 eq.) at −65° C. After 30 min, a solution of 5-chlorothieno[2,3-b]pyridine-3-carbonitrile (270 mg, 1.39 mmol, 1.00 eq.) in THF (3.0 mL) was added to the above solution at −65° C. drop wise, and the resulting mixture was stirred at −40° C. for 1 hour. The solution was cooled to −65° C., and a solution of iodine (528 mg, 2.08 mmol, 419 μL, 1.50 eq.) in THF (3.0 mL) was added to above mixture drop wise. The mixture was warmed to 25° C. and stirred for 12 hours. The reaction mixture was quenched with sat. aqueous ammonium chloride (15 mL), stirred for 15 min under nitrogen atmosphere, then extracted with ethyl acetate (50 mL×3), washed with aqueous sodium sulfite (50 mL), brine (50 mL), dried over sodium sulfate, filtered and concentrated under reduced pressure to give 5-chloro-2-iodo-thieno[2,3-b]pyridine-3-carbonitrile (440 mg, 91% yield) as a yellow solid. LCMS [M+1]⁺=320.9. 1H NMR (400 MHz, CDCl₃-d) δ=8.58 (d, J=2.4 Hz, 1H), 8.17 (d, J=2.4 Hz, 1H).

Step 2: A mixture of 1-methyl-5-(4,4,5,5-tetramethyl-1,3,2-dioxaborolan-2-yl)pyrazole (343 mg, 1.65 mmol, 1.20 eq.), 5-chloro-2-iodo-thieno[2,3-b]pyridine-3-carbonitrile (440 mg, 1.37 mmol, 1.00 eq.), sodium bicarbonate (346 mg, 4.12 mmol, 160 μL, 3.00 eq.), Pd(dtbpf)Cl₂ (89.5 mg, 137 μmol, 0.10 eq.) in dioxane (10.0 mL) and water (2.0 mL) was purged with nitrogen for 3 times and stirred at 80° C. for 2 hours under nitrogen atmosphere. The mixture was concentrated under reduced pressure and the residue was purified by column chromatography (SiO₂, Petroleum ether/Ethyl acetate 5-20%) to give 5-chloro-2-(2-methylpyrazol-3-yl)thieno[2,3-b]pyridine-3-carbonitrile (140 mg, 32% yield) as a yellow solid.

LCMS [M+1]⁺=275.0. ¹H NMR (400 MHz, CDCl₃-d) δ=8.67 (d, J=2.4 Hz, 1H), 8.26 (d, J=2.4 Hz, 1H), 7.66 (d, J=2.0 Hz, 1H), 6.87 (d, J=2.0 Hz, 1H), 4.13 (s, 3H).

Step 3: A mixture of 5-chloro-2-(2-methylpyrazol-3-yl)thieno[2,3-b]pyridine-3-carbonitrile (120 mg, 437 μμmol, 1.00 eq.), N-Iodosuccinimide (491 mg, 2.18 mmol, 5.00 eq.) in acetic acid (8.0 mL) was degassed and stirred at 80° C. for 3 hours under nitrogen atmosphere. The reaction mixture was extracted with ethyl acetate (15 mL×3). The combined organic layers were washed aqueous with sodium sulfite (15 mL×2), brine (15 mL), dried over sodium sulfate, filtered and concentrated under reduced pressure to give 5-chloro-2-(4-iodo-2-methyl-pyrazol-3-yl)thieno[2,3-b]pyridine-3-carbonitrile (170 mg, 68% yield, 70% purity) as a yellow solid. LCMS [M+1]⁺=400.9. 1 H NMR (400 MHz, CDCl₃) δ=8.74 (d, J=2.4 Hz, 1H), 8.33 (d, J=2.4 Hz, 1H), 7.72 (s, 1H), 4.03 (s, 3H).

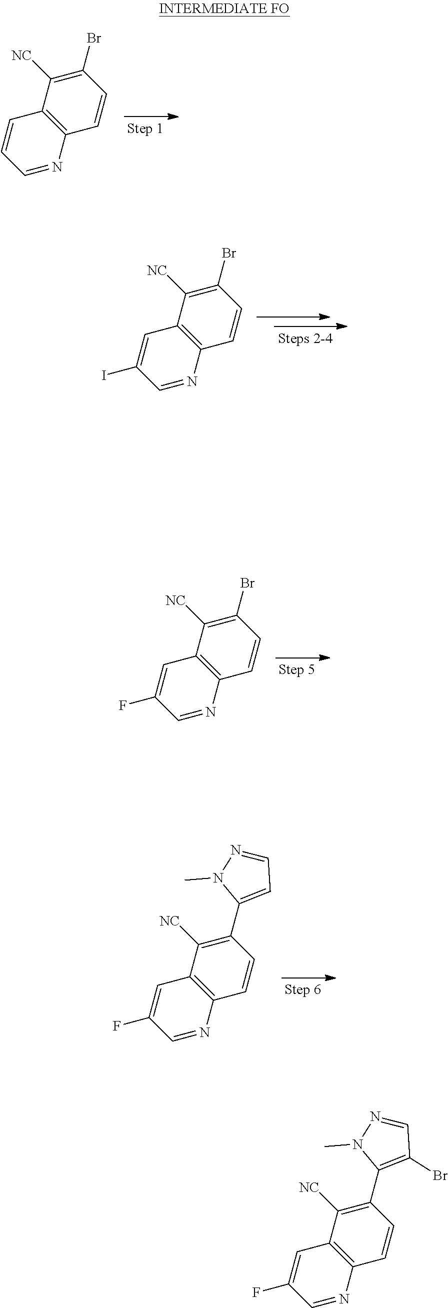 [SMILES: N#Cc1c(Br)ccc2ncccc12 ; N#Cc1c(Br)ccc2ncc(I)cc12 ; N#Cc1c(Br)ccc2ncc(F)cc12 ; Cn1nccc1-c1ccc2ncc(F)cc2c1C#N ; Cn1ncc(Br)c1-c1ccc2ncc(F)cc2c1C#N]

Step 1: To a mixture of 6-bromoquinoline-5-carbonitrile (1.30 g, 5.58 mmol, 1.00 eq.) in acetonitrile (20 mL) was added iodine (4.25 g, 16.7 mmol, 3.00 eq.) and 2-hydroperoxy-2-methyl-propane (2.15 g, 16.7 mmol, 2.29 mL, 70% purity, 3.00 eq.) at 30° C. The reaction mixture was stirred at 80° C. for 16 hours. The mixture was poured into water (40 mL), quenched by saturated sodium sulfite (50 mL), extracted with ethyl acetate (30 mL×4). The combined organic layers were washed with brine (50 mL), dried over sodium sulfate, filtered and concentrated in vacuum to give the residue. The residue was purified by flash silica gel chromatography (Ethyl acetate/petroleum 0-100%) to afford 6-bromo-3-iodo-quinoline-5-carbonitrile (460 mg, 22% yield) as a yellow solid. LCMS [M+1]⁺: 358.9. ¹H NMR (400 MHz, CDCl₃) δ9.15 (d, J=2.0 Hz, 1H), 8.96-8.84 (m, 1H), 8.14 (d, J=9.2 Hz, 1H), 7.93 (d, J=9.2 Hz, 1H).

Step 2: A mixture of 6-bromo-3-iodo-quinoline-5-carbonitrile (300 mg, 0.83 mmol, 1.00 eq.), tert-butyl carbamate (97.9 mg, 0.83 mmol, 1.00 eq.), cesium carbonate (545 mg, 1.67 mmol, 2.00 eq.), 4,5-Bis(diphenylphosphino)-9,9-dimethylxanthene (48.0 mg, 0.08 mmol, 0.10 eq.) and tris(dibenzylideneacetone)dipalladium(0) (38.0 mg, 0.04 mmol, 0.05 eq) in dioxane (15 mL) was degassed and stirred at 85° C. for 1.5 hours under nitrogen atmosphere. The reaction mixture was washed with water (60 mL), extracted with ethyl acetate (20 mL×3). The combined organic layers were dried over sodium sulfate, filtered and concentrated. The residue was purified by flash silica gel chromatography (Ethyl acetate/Petroleum ether 0-80%) to afford tert-butyl N-(6-bromo-5-cyano-3-quinolyl)-carbamate (270 mg, 91% yield) as a yellow solid. LCMS [M+3]⁺: 350.0. ¹H NMR (400 MHz, CDCl₃) δ8.93 (d, J=1.6 Hz, 1H), 8.62 (d, J=2.0 Hz, 1H), 8.08 (d, J=9.2 Hz, 1H), 7.77 (d, J=9.2 Hz, 1H), 7.08 (s, 1H), 1.58 (s, 9H).

Step 3: To a mixture of tert-butyl N-(6-bromo-5-cyano-3-quinolyl) carbamate (270 mg, 0.76 mmol, 1.00 eq.) in dioxane (2.0 mL) was added hydrochloric acid/dioxane (4.00 M, 3.0 mL, 15.4 eq.). The reaction mixture was stirred at 30° C. for 16 hours. The reaction mixture was concentrated, and then adjusted to pH=8 by saturated sodium bicarbonate, and extracted by chroroform/isopropanol 3/1 (6 mL×3). The combined organic layers were dried over sodium sulfate, filtered and concentrated to afford 3-amino-6-bromo-quinoline-5-carbonitrile (170 mg, 88% yield) as a yellow solid. LCMS [M+3]⁺: 250.0. ¹H NMR (400 MHz, DMSO-d₆) δ 8.57 (s, 1H), 7.96 (d, J=8.8 Hz, 1H), 7.62 (d, J=8.8 Hz, 1H), 7.25 (s, 1H), 6.53 (s, 2H).

Step 4: To a mixture of 3-amino-6-bromo-quinoline-5-carbonitrile (170 mg, 0.69 mmol, 1.00 eq.) in pyridine hydrofluoride (4.03 g, 28.4 mmol, 3.70 mL, 70% purity, 41.5 eq.) at −40° C. under nitrogen atmosphere was added sodium nitrite (71.0 mg, 1.03 mmol, 1.50 eq.). The reaction mixture was warmed to 30° C. and stirred for 0.5 h under nitrogen atmosphere followed by stirring at 80° C. for 2 h. The reaction mixture was dissolved in dimethyl formamide (20 mL), filtered and the filtrate was purified by reversed-phase HPLC (column: 330 g Flash Column Welch ultimate XB_C18 20-40 μm; 120 A; condition: water (0.1% formic acid)-acetonitrile; B %=15 to 60, 10 min). The eluent containing desired product was concentrated to remove acetonitrile and then extracted with dichloromethane (20 mL×3). The combined organic layers were dried over sodium sulfate, filtered and concentrated to afford 6-bromo-3-fluoro-quinoline-5-carbonitrile (140 mg, 80% yield) as a yellow solid. LCMS [M+3]⁺: 253.0. ¹H NMR (400 MHz, CDCl₃) δ8.96-8.90 (m, 1H), 8.21 (d, J=9.2 Hz, 1H), 8.14-8.12 (m, 1H), 7.91 (d, J=9.2 Hz, 1H).

Step 5: A mixture of 6-bromo-3-fluoro-quinoline-5-carbonitrile (140 mg, 0.55 mmol, 1.00 eq.), 1-methyl-5-(4,4,5,5-tetramethyl-1,3,2-dioxaborolan-2-yl) pyrazole (348 mg, 1.67 mmol, 3.00 eq.), sodium carbonate (177 mg, 1.67 mmol, 3.00 eq.) and [1,1′-bis(diphenylphosphino)ferrocene]dichloropalladium(II) dichloromethane (23.0 mg, 27.8 μmol, 0.05 eq.) in dioxane (6.0 mL) and water (1.20 mL) was purged with nitrogen stirred at 80° C. for 2 hours under nitrogen atmosphere. The reaction mixture was diluted with water (10.0 mL), extracted with dichloromethane (5.0 mL×3). The combined organic layers were dried over sodium sulfate, filtered and concentrated. The residue was purified by flash silica gel chromatography (Ethyl acetate/Petroleum ether 0-100%) to afford 3-fluoro-6-(2-methylpyrazol-3-yl)quinoline-5-carbonitrile (90.0 mg, 63.2% yield) as a white solid. LCMS [M+1]⁺: 253.2. ¹H NMR (400 MHz, CDCl₃) δ8.99 (d, J=2.8 Hz, 1H), 8.44 (d, J=8.4 Hz, 1H), 8.26-8.23 (m, 1H), 7.75 (d, J=8.8 Hz, 1H), 7.66 (d, J=1.2 Hz, 1H), 6.63 (d, J=1.2 Hz, 1H), 3.92 (s, 3H).

Step 6: To a mixture of 3-fluoro-6-(2-methylpyrazol-3-yl)quinoline-5-carbonitrile (90.0 mg, 0.35 mmol, 1.00 eq) in dimethyl formamide (3.0 mL) was added N-bromosuccinimide (95.0 mg, 0.54 mmol, 1.50 eq). The reaction mixture was stirred at 20° C. for 3 hours. The reaction mixture was washed with water (15 mL), extracted with ethyl acetate (5 mL×3). The combined organic layers were washed with brine (20 mL), dried over sodium sulfate, filtered and concentrated to afford 6-(4-bromo-2-methyl-pyrazol-3-yl)-3-fluoro-quinoline-5-carbonitrile (100 mg, 83% yield) as a yellow oil. LCMS [M+3]⁺: 333.0. ¹H NMR (400 MHz, CDCl₃) δ9.02 (d, J=2.8 Hz, 1H), 8.49 (d, J=8.8 Hz, 1H), 8.27-8.24 (m, 1H), 7.73 (d, J=8.8 Hz, 1H), 7.65 (s, 1H), 3.86 (s, 3H);

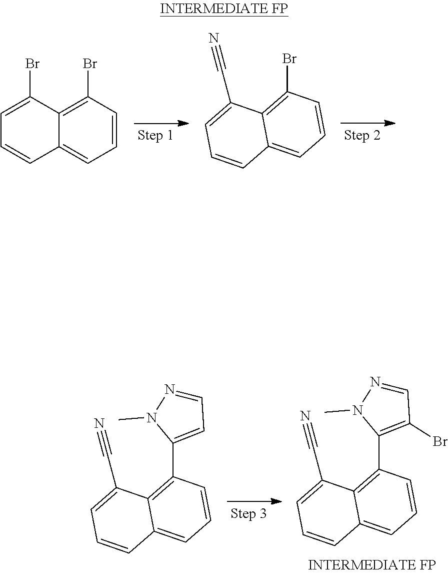 [SMILES: Brc1cccc2cccc(Br)c12 ; N#Cc1cccc2cccc(Br)c12 ; Cn1nccc1-c1cccc2cccc(C#N)c12 ; Cn1ncc(Br)c1-c1cccc2cccc(C#N)c12]

Step 1: A mixture of 1,8-dibromonaphthalene (5.00 g, 17.5 mmol, 1.00 eq.) and cuprous cyanide (1.72 g, 19.2 mmol, 4.20 mL, 1.10 eq.) in dimethyl formamide (10.0 mL) was degassed and stirred at 140° C. for 4 hours under nitrogen atmosphere. The mixture was concentrated under reduced pressure and the residue was purified by column chromatography (SiO₂, Ethyl acetate/Petroleum ether 2-20%) to give 8-bromonaphthalene-1-carbonitrile (2.10 g, 52% yield) as a yellow solid. ¹H NMR (400 MHz, CDCl₃) δ=8.11 (m, 1H), 8.09 (d, J=1.6 Hz, 1H), 7.96 (dd, J=1.2, 7.6 Hz, 1H), 7.90 (dd, J=1.2, 8.4 Hz, 1H), 7.57 (dd, J=7.6, 8.4 Hz, 1H), 7.43 (t, J=8.0 Hz, 1H).

Step 2: A mixture of 1-methyl-5-(4,4,5,5-tetramethyl-1,3,2-dioxaborolan-2-yl)pyrazole (524 mg, 2.52 mmol, 1.30 eq.), 8-bromonaphthalene-1-carbonitrile (450 mg, 1.94 mmol, 1.00 eq.), Pd(dtbpf)Cl₂ (126 mg, 194 μμmol, 0.10 eq.) and sodium bicarbonate (489 mg, 5.82 mmol, 226 μL, 3.00 eq.) in dioxane (10.0 mL) and water (2.0 mL) was purged with nitrogen for 3 times and stirred at 80° C. for 1 hour under nitrogen atmosphere. The mixture was concentrated under reduced pressure and the residue was purified by column chromatography (SiO₂, Ethyl acetate/Petroleum ether 10-35%) to give 8-(2-methylpyrazol-3-yl)naphthalene-1-carbonitrile (400 mg, 88% yield) as a white solid. LCMS [M+1]⁺=234.0. ¹H NMR (400 MHz, CDCl₃) δ=8.19 (dd, J=1.2, 8.4 Hz, 1H), 8.04 (ddd, J=1.2, 7.6, 16.6 Hz, 2H), 7.72-7.65 (m, 2H), 7.64-7.57 (m, 2H), 6.47-6.37 (m, 1H), 3.63 (s, 3H).

Step 3: A mixture of 8-(2-methylpyrazol-3-yl)naphthalene-1-carbonitrile (120 mg, 514 μμmol, 1.00 eq.), N-bromosuccinimide (274 mg, 1.54 mmol, 3 eq) in acetonitrile (2.0 mL) was degassed stirred at 60° C. for 2 hours under nitrogen atmosphere. The mixture was concentrated under reduced pressure and the residue was purified by prep-TLC (SiO₂, Ethyl acetate/Petroleum ether 1:3) to give 8-(4-bromo-2-methyl-pyrazol-3-yl)naphthalene-1-carbonitrile (110 mg, 67% yield) as a yellow solid. LCMS [M+1]⁺=313.9. 1H NMR (400 MHz, CDCl₃) δ=8.21 (dd, J=1.2, 8.4 Hz, 1H), 8.11 (dd, J=1.2, 8.4 Hz, 1H), 8.03 (dd, J=1.6, 7.2 Hz, 1H), 7.74 (dd, J=7.2, 8.4 Hz, 1H), 7.67-7.65 (m, 1H), 7.61 (dt, J=1.2, 7.6 Hz, 2H), 3.67 (s, 3H).

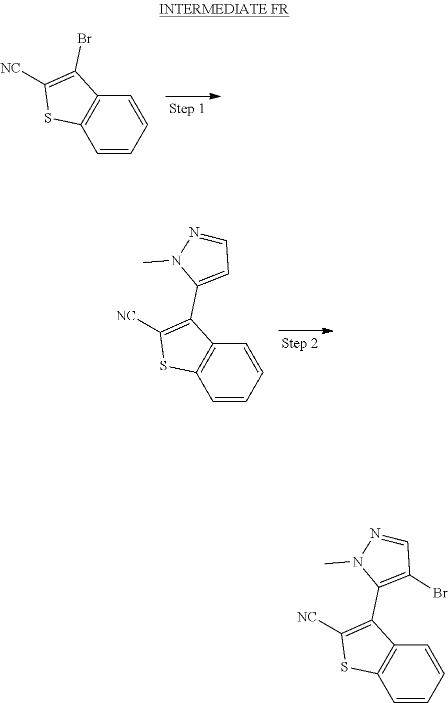 [SMILES: N#Cc1sc2ccccc2c1Br ; Cn1nccc1-c1c(C#N)sc2ccccc12 ; Cn1ncc(Br)c1-c1c(C#N)sc2ccccc12]

Step 1: A mixture of 1-methyl-5-(4,4,5,5-tetramethyl-1,3,2-dioxaborolan-2-yl)pyrazole (1.42 g, 6.80 mmol, 1.20 eq), 3-bromobenzothiophene-2-carbonitrile (1.35 g, 5.67 mmol, 1.00 eq.), Pd(dtbpf)Cl₂ (370 mg, 567 μμmol, 0.10 eq.) and sodium bicarbonate (1.43 g, 17.0 mmol, 662 μL, 3.00 eq.) in dioxane (15 mL) and water (3.0 mL) was degassed stirred at 80° C. for 2 hours under nitrogen atmosphere. The mixture was concentrated under reduced pressure and the residue was purified by column chromatography (SiO₂, Ethyl acetate/Petroleum ether 3-20%) to give 3-(2-methylpyrazol-3-yl)benzothiophene-2-carbonitrile (1.00 g, 68% yield) as a yellow solid. LCMS [M+1]⁺=240.1. ¹H NMR (400 MHz, CDCl₃)=7.94 (d, J=8.4 Hz, 1H), 7.71-7.67 (m, 2H), 7.62 (ddd, J=1.2, 7.2, 8.4 Hz, 1H), 7.56-7.50 (m, 1H), 6.56 (d, J=2.0 Hz, 1H), 3.87 (s, 3H)

Step 2: A mixture of 3-(2-methylpyrazol-3-yl)benzothiophene-2-carbonitrile (500 mg, 2.09 mmol, 1.00 eq.) and N-bromosuccinimide (446 mg, 2.51 mmol, 1.20 eq.) in acetonitrile (5.0 mL) was degassed and stirred at 40° C. for 2 hours under nitrogen atmosphere. The mixture was concentrated under reduced pressure to give 3-(4-bromo-2-methyl-pyrazol-3-yl)benzothiophene-2-carbonitrile (350 mg, crude) as a yellow solid. LCMS [M+1]⁺=319.9. ¹H NMR (400 MHz, CDCl₃-d) δ=7.96 (d, J=8.4 Hz, 1H), 7.70 (s, 1H), 7.64 (ddd, J=2.0, 6.4, 8.4 Hz, 1H), 7.60-7.51 (m, 2H), 3.82 (s, 3H).

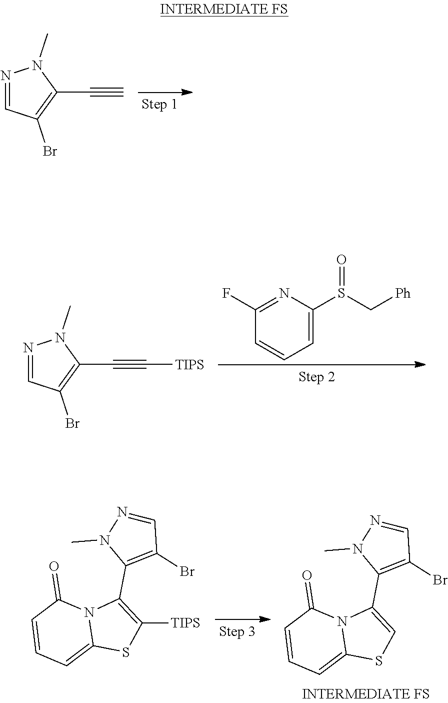

Step 1: To a solution of 4-bromo-5-ethynyl-1-methyl-pyrazole (1.50 g, 8.11 mmol, 1.00 eq.) in THF (20 mL) at −78° C. under nitrogen atmosphere was added lithium diisopropyl amide (2.00 M, 6.10 mL, 1.50 eq.) and the resulting mixture was stirred for 0.5 hours. Triisopropylsilylchloride (2.34 g, 12.1 mmol, 2.60 mL, 1.50 eq.) was added at −60° C. under nitrogen atmosphere. After the addition was completed, the reaction mixture was warmed to 30° C. and stirred at 30° C. for 0.5 hours under nitrogen atmosphere. The reaction mixture was quenched by saturated ammonium chloride solution (50 mL), extracted with ethyl acetate (25 mL×3). The combined organic layers were dried over sodium sulfate, filtered and concentrated. The residue was purified by flash silica gel chromatography (Ethyl acetate/Petroleum ether 0-20%) to afford 2-(4-bromo-2-methyl-pyrazol-3-yl)ethynyl-triisopropyl-silane (1.00 g, 36% yield) as colorless oil. LCMS [ESI, M+1]⁺: 343.1. ¹H NMR (400 MHz, MeOD) δ 7.48 (s, 1H), 3.92 (s, 3H), 1.19-1.14 (m, 21H).

Step 2: To a mixture of 2-benzylsulfinyl-6-fluoro-pyridine (700 mg, 2.98 mmol, 1.00 eq.) in dichloromethane (50 mL) was added 2-(4-bromo-2-methyl-pyrazol-3-yl)ethynyl-triisopropyl-silane (1.52 g, 4.46 mmol, 1.50 eq.) and 2-fluoropyridine (289 mg, 2.98 mmol, 0.30 mL, 1.00 eq.) at 30° C. under nitrogen atmosphere. The mixture was cooled to −60° C. and stirred for 0.1 hours before trifluoromethanesulfonic anhydride (1.26 g, 4.46 mmol, 0.70 mL, 1.50 eq., redistilled) was added dropwise under nitrogen atmosphere. After the addition was completed, the reaction mixture was warmed to 30° C. and stirred at 30° C. for 16 hours. Sodium carbonate (5.93 g, 55.9 mmol, 18.8 eq.) was added and the resulting mixture was stirred at 30° C. for 3 hours. The reaction mixture was washed with water (100 mL) and extracted with dichloromethane (30 mL×3). The combined organic layers were washed with brine (100 mL), dried over sodium sulfate, filtered and concentrated. The residue was purified by flash silica gel chromatography (Ethyl acetate/Petroleum ether 0-100%) to afford 3-(4-bromo-2-methyl-pyrazol-3-yl)-2-triisopropylsilyl-thiazolo [3,2-a]pyridin-5-one (850 mg, 37% yield) as a white solid. LCMS [ESI, M+1]⁺: 466.1. ¹H NMR (400 MHz, CDCl₃) δ7.48 (s, 1H), 7.40-7.36 (m, 1H), 6.64-6.62 (m, 1H), 6.21-6.19 (m, 1H), 3.73 (s, 3H), 1.20-1.02 (m, 21H).

Step 3: To a mixture of 3-(4-bromo-2-methyl-pyrazol-3-yl)-2-triisopropylsilyl-thiazolo[3,2-a]pyridin-5-one (500 mg, 1.07 mmol, 1.00 eq.) in THF (10.0 mL) at −40° C. under nitrogen atmosphere was added tetrabutylammonium fluoride (1.00 M, 4.30 mL, 4.00 eq.). The reaction mixture was stirred at 0.25 hours. The reaction mixture was directly purified by flash silica gel chromatography (Ethyl acetate/Petroleum ether 0-100%) and further purified by Prep-TLC (100% Ethyl acetate) to afford 3-(4-bromo-2-methyl-pyrazol-3-yl)thiazolo[3,2-a]pyridin-5-one (250 mg, 75% yield) as a white solid. LCMS [ESI, M+1]⁺: 312.0. ¹H NMR (400 MHz, CDCl₃) δ7.51 (s, 1H), 7.46 (dd, J=7.6, 9.2 Hz, 1H), 7.00 (s, 1H), 6.70 (dd, J=0.8, 7.6 Hz, 1H), 6.31 (dd, J=0.8, 8.8 Hz, 1H), 3.76 (s, 3H).

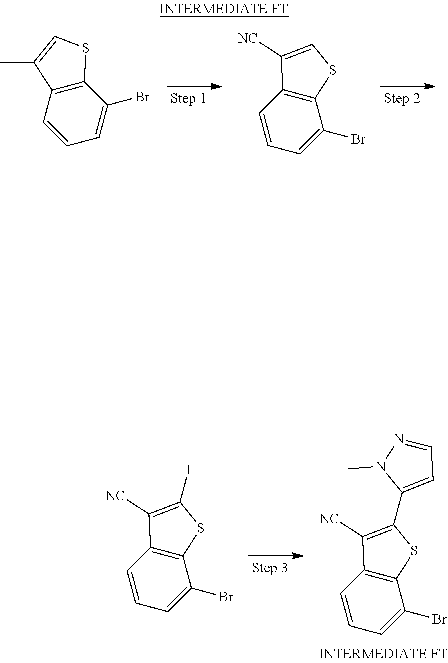 [SMILES: Cc1csc2c(Br)cccc12 ; N#Cc1csc2c(Br)cccc12 ; N#Cc1c(I)sc2c(Br)cccc12 ; Cn1nccc1-c1sc2c(Br)cccc2c1C#N]

Step 1: To a solution of 7-bromo-3-methyl-benzothiophene (9.40 g, 41.4 mmol, 1.00 eq.) and 2-hydroperoxy-2-methyl-propane (47.0 g, 522 mmol, 50 mL, 12.6 eq.) in dimethylsulfoxide (90.0 mL) was added iodine (15.0 g, 41.4 mmol, 11.9 mL, 70% purity, 1.00 eq.) and ammonium fluoride (6.13 g, 166 mmol, 4.00 eq.). The mixture was stirred at 70° C. for 12 hours under oxygen atmosphere. The mixture was treated with saturated sodium sulfite (50 mL) stirred at 0° C. for 10 mins, diluted with water (100 mL) and extracted with ethyl acetate (50 mL×3). The combined organic layers were washed with brine (30 mL), dried over anhydrous sodium sulfate, filtered and concentrated under reduced pressure and the residue was further purified by flash silica gel chromatography (Ethyl acetate/Petroleum ether 0-20%) to give 7-bromobenzothiophene-3-carbonitrile (4.8 g, 39% yield) as a yellow solid. ¹H NMR (400 MHz, CDCl₃) δ=8.19 (s, 1H), 7.97 (d, J=8.0 Hz, 1H), 7.65 (d, J=8.0 Hz, 1H), 7.45 (t, J=8.0 Hz, 1H).

Step 2: A solution of dry N-isopropylpropan-2-amine (1.91 g, 18.9 mmol, 2.67 mL, 1.50 eq.) in 3,4-dimethylTHF (15 mL) was purged with nitrogen for 3 times at −70° C. and treated with n-butyllithium (2.5 M, 7.56 mL, 1.5 eq.) drop-wise. The mixture was stirred at this temperature for 30 mins before 7-bromobenzothiophene-3-carbonitrile (3.00 g, 12.6 mmol, 1.00 eq.) in 3,4-dimethylTHF (30 mL) was added drop-wise at −70° C., followed by iodine (6.40 g, 25.2 mmol, 5.08 mL, 2.00 eq.) in 3,4-dimethylTHF (15 mL). The resulting mixture was stirred at 25° C. for 12 hours. The mixture was quenched with saturated ammonium chloride (30 mL), diluted with water (70 mL) and extracted with ethyl acetate (50 mL×3). The organic layers were washed with brine (50 mL), dried over anhydrous sodium sulfate, filtered and concentrated under reduced pressure and the crude product was purified by re-crystallization from methanol (20 mL) at 20° C. for 0.5 hours to give 7-bromo-2-iodo-benzothiophene-3-carbonitrile (4.10 g, 89% yield) as a brown solid. ¹H NMR (400 MHz, CDCl₃-d) δ=7.89-7.85 (m, 1H), 7.57 (dd, J=0.8, 8.0 Hz, 1H), 7.40-7.35 (m, 1H).

Step 3: A mixture of 7-bromo-2-iodo-benzothiophene-3-carbonitrile (2.00 g, 5.49 mmol, 1.00 eq.), 1-methyl-5-(4,4,5,5-tetramethyl-1,3,2-dioxaborolan-2-yl)pyrazole (1.20 g, 5.77 mmol, 1.05 eq), sodium bicarbonate (914 mg, 10.9 mmol, 423 μL, 1.98 eq.), [1,1-bis(diphenylphosphino)ferrocene]dichloropalladium(II) (402 mg, 549 μmol, 0.10 eq.) in water (3.0 mL) and dioxane (30 mL) was purged with nitrogen for 3 times and stirred at 80° C. for 3 hours under nitrogen atmosphere. After being cooled to room temperature, the mixture was concentrated under vacuum. The residue was purified by flash silica gel chromatography (Ethyl acetate/Petroleum ether 0-30%) to give 7-bromo-2-(2-methylpyrazol-3-yl)benzothiophene-3-carbonitrile (1.20 g, 3.55 mmol, 65% yield) as a yellow solid. ¹H NMR (400 MHz, CDCl₃) δ=7.96 (d, J=8.0 Hz, 1H), 7.66 (d, J=7.8 Hz, 1H), 7.62 (d, J=2.0 Hz, 1H), 7.47 (t, J=7.8 Hz, 1H), 6.80 (d, J=2.0 Hz, 1H), 4.10 (s, 3H).

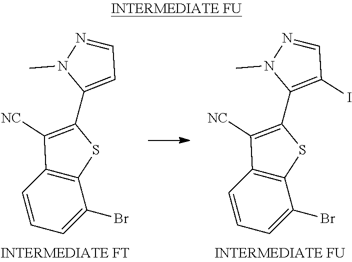 [SMILES: Cn1nccc1-c1sc2c(Br)cccc2c1C#N ; Cn1ncc(I)c1-c1sc2c(Br)cccc2c1C#N]

To a solution of 7-bromo-2-(2-methylpyrazol-3-yl)benzothiophene-3-carbonitrile (1.20 g, 3.77 mmol, 1.00 eq.) in acetonitrile (30 mL) was added N-iodosuccinimide (4.24 g, 18.9 mmol, 5.00 eq.) and acetic acid (2.10 g, 35.0 mmol, 2.0 mL, 9.27 eq.). The mixture was stirred at 50° C. for 12 hours. After being cooled to room temperature, the mixture was concentrated under vacuum. The residue was diluted with ethyl acetate (50 mL) and water (30 mL). The layers were separated and the aqueous phase was extracted with ethyl acetate (3×30 mL). The combined organic layers were dried over sodium sulfate, filtered and concentrated in vacuum to give 7-bromo-2-(4-iodo-2-methyl-pyrazol-3-yl)benzothiophene-3-carbonitrile (1.30 g, 74% yield) as a yellow solid, which was used into next step directly without further purification.

¹H NMR (400 MHz, CDCl₃) δ=8.02 (dd, J=0.8, 8.0 Hz, 1H), 7.75-7.66 (m, 2H), 7.51 (t, J=8.0 Hz, 1H), 3.99 (s, 3H).

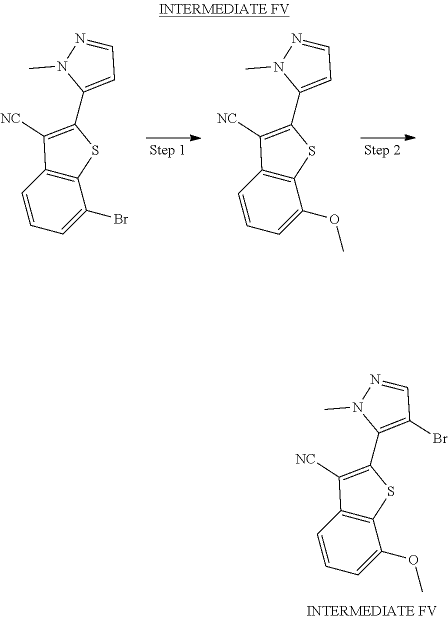 [SMILES: Cn1nccc1-c1sc2c(Br)cccc2c1C#N ; COc1cccc2c(C#N)c(-c3ccnn3C)sc12 ; COc1cccc2c(C#N)c(-c3c(Br)cnn3C)sc12]

Step 1: A mixture of 7-bromo-2-(2-methylpyrazol-3-yl)benzothiophene-3-carbonitrile (300 mg, 943 μμmol, 1.00 eq.), cuprous iodide (35.9 mg, 189 μμmol, 0.20 eq.), sodium methoxide (255 mg, 4.71 mmol, 5.00 eq.) in N,N-dimethylformamide (10.0 mL) was purged with nitrogen for 3 times and stirred at 100° C. for 4 hours under nitrogen atmosphere. The reaction mixture was diluted with water (30 mL) and extracted with ethyl acetate (30 mL×3). The combined organic layers were dried over sodium sulfate, filtered and concentrated in vacuum and the residue was purified by column chromatography by prep-TLC (SiO₂, Petroleum ether/Ethyl acetate 3:2) to give 7-hydroxy-2-(2-methylpyrazol-3-yl)benzothiophene-3-carbonitrile (15.0 mg, 6% yield) as a white solid and 7-methoxy-2-(2-methylpyrazol-3-yl)benzothiophene-3-carbonitrile (95 mg, 37% yield) as a yellow solid. LCMS [M+1]⁺=269.9. ¹H NMR (400 MHz, CDCl₃) δ=7.63-7.60 (m, 3H), 7.65-7.58 (m, 1H), 6.80-6.79 (m, 1H), 4.10 (s, 3H), 4.05 (s, 3H).

Step 2: To a solution of 7-methoxy-2-(2-methylpyrazol-3-yl)benzothiophene-3-carbonitrile (78.0 mg, 261 μμmol, 90% purity, 1.00 eq.) in acetonitrile (8.0 mL) was added N-bromosuccinimide (46.4 mg, 261 μμmol, 1.00 eq.). The mixture was stirred at 20° C. for 12 hours. The mixture was concentrated and the crude material was purified by prep-HPLC column: Phenomenex Luna C18 150×25 mm×10 μm; mobile phase: [water (0.1% TFA)-ACN]; B %: 51%-81%, 10 min to give 2-(4-bromo-2-methyl-pyrazol-3-yl)-7-methoxy-benzothiophene-3-carbonitrile (60 mg, 66% yield) as a white solid. LCMS [M+3]⁺=349.9. ¹H NMR (400 MHz, CDCl₃) δ=7.65 (d, J=8.0 Hz, 1H), 7.63 (s, 1H), 7.55 (t, 1=8.0 Hz, 1H), 6.97 (d, J=8.0 Hz, 1H), 4.05 (s, 3H), 3.96 (s, 3H).

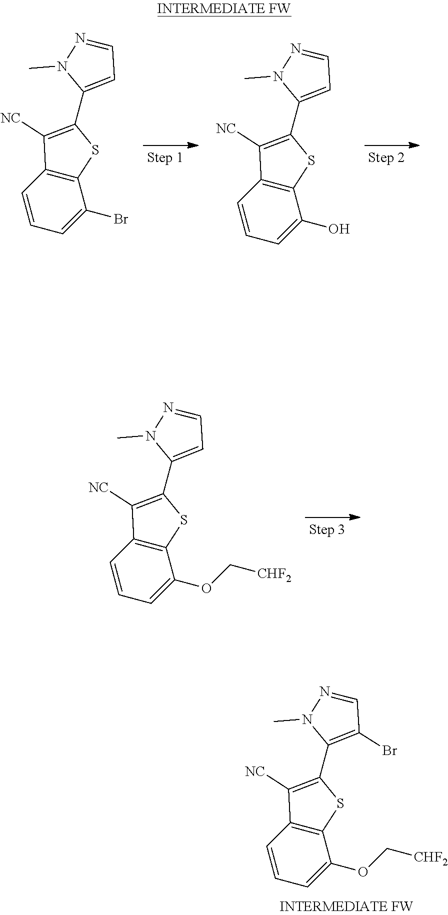 [SMILES: Cn1nccc1-c1sc2c(Br)cccc2c1C#N ; Cn1nccc1-c1sc2c(O)cccc2c1C#N ; Cn1nccc1-c1sc2c(OCC(F)F)cccc2c1C#N ; Cn1ncc(Br)c1-c1sc2c(OCC(F)F)cccc2c1C#N]

Step 1: A mixture of 7-bromo-2-(2-methylpyrazol-3-yl) benzothiophene-3-carbonitrile (700 mg, 2.20 mmol, 1.00 eq.), copper iodide (83.8 mg, 440 μμmol, 0.20 eq.), sodium methoxide (594 mg, 11.0 mmol, 5.00 eq.) in N,N-dimethylformamide (20 mL) was purged with nitrogen for 3 times and stirred at 100° C. for 2 hours under nitrogen atmosphere. The reaction mixture was diluted with water (30 mL) and extracted with ethyl acetate (30 mL×3), dried over anhydrous sodium sulfate. The combined organic phase was concentrated in vacuum and the residue was purified by column chromatography (SiO₂, Ethyl acetate/Petroleum 20-50%) to give 7-methoxy-2-(2-methylpyrazol-3-yl) benzothiophene-3-carbonitrile (200 mg, 33% yield) as a white solid and 7-hydroxy-2-(2-methylpyrazol-3-yl)benzothiophene-3-carbonitrile (275 mg, 49% yield) as a white solid. ¹H NMR (400 MHz, CDCl₃-d) δ=7.66-7.60 (m, 2H), 7.44 (t, J=8.0 Hz, 1H), 6.90 (d, J=8.0 Hz, 1H), 6.80 (d, J=2.0 Hz, 1H), 4.10 (s, 3H).

Step 2: To a solution of 7-hydroxy-2-(2-methylpyrazol-3-yl) benzothiophene-3-carbonitrile (100 mg, 39.2 μμmol, 1.00 eq.) in N,N-dimethylformamide (5.0 mL) was added potassium carbonate (108 mg, 781 μμmol, 1.99 eq.) and 1,1-difluoro-2-iodo-ethane (153 mg, 795 μμmol, 2.03 eq.). The mixture was stirred at 100° C. for 2 hours. After being cooled to room temperature, the mixture was concentrated under vacuum. The residue was diluted with water (20 mL) and extracted with ethyl acetate (20 mL). The layers were separated and the aqueous phase was extracted with ethyl acetate (3×20 mL). The combined organic layers were dried over anhydrous sodium sulfate, filtered and concentrated under vacuum to give 7-(2, 2-difluoroethoxy)-2-(2-methylpyrazol-3-yl) benzothiophene-3-carbonitrile (100 mg, 80% yield) as a yellow solid. ¹H NMR (400 MHz, CDCl₃) δ=7.68 (d, J=8.0 Hz, 1H), 7.62 (d, J=1.6 Hz, 1H), 7.53 (t, J=8.0 Hz, 1H), 6.94 (d, J=8.0 Hz, 1H), 6.80 (s, 1H), 6.40-6.02 (m, 1H), 4.43 (dt, J=4.0, 12.8 Hz, 2H), 4.09 (s, 3H).

Step 3: To a solution of 7-(2, 2-difluoroethoxy)-2-(2-methylpyrazol-3-yl) benzothiophene-3-carbonitrile (50 mg, 157 μμmol, 1.00 eq.) in acetonitrile (2.0 mL) was added N-bromosuccinimide (27.0 mg, 152 μμmol, 0.10 eq.). The mixture was stirred at 15° C. for 12 hours. The mixture was concentrated under reduced pressure and the residue was purified by column chromatography on silica gel (Petroleum ether/Ethyl acetate 2:1) to give 2-(4-bromo-2-methyl-pyrazol-3-yl)-7-(2, 2-difluoroethoxy) benzothiophene-3-carbonitrile (30.0 mg, 48% yield) as a yellow solid. ¹H NMR (400 MHz, CDCl₃-d) δ=7.73 (d, J=8.0 Hz, 1H), 7.64 (s, 1H), 7.56 (t, J=8.0 Hz, 1H), 6.98 (d, J=8.0 Hz, 1H), 6.38-6.04 (m, 1H), 4.44 (dt, J=4.0, 12.8 Hz, 2H), 3.96 (s, 3H).

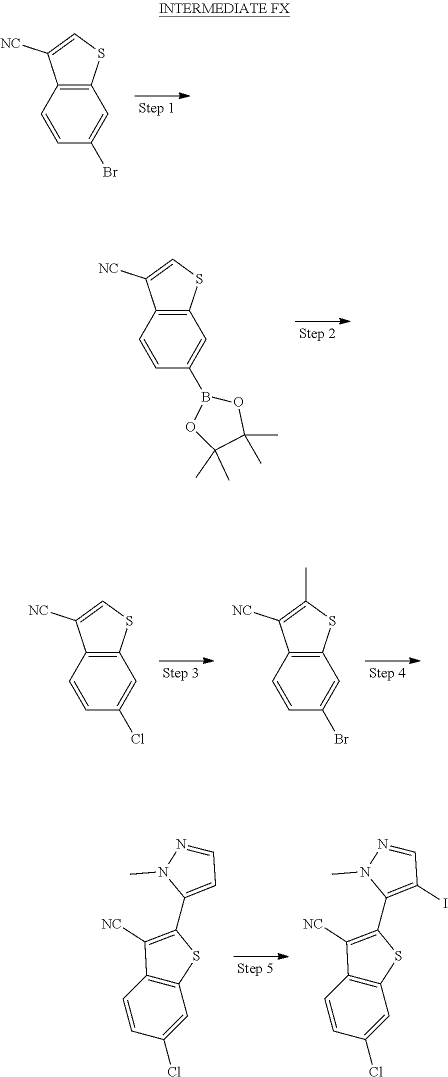 [SMILES: N#Cc1csc2cc(Br)ccc12 ; CC1(C)OB(c2ccc3c(C#N)csc3c2)OC1(C)C ; N#Cc1csc2cc(Cl)ccc12 ; Cc1sc2cc(Br)ccc2c1C#N ; Cn1nccc1-c1sc2cc(Cl)ccc2c1C#N ; Cn1ncc(I)c1-c1sc2cc(Cl)ccc2c1C#N]

Step 1: To a solution of 6-bromobenzo[b]thiophene-3-carbonitrile (750 mg, 3.15 mmol, 1.00 eq.), 4,4,5,5-tetramethyl-2-(4,4,5,5-tetramethyl-1,3,2-dioxaborolan-2-yl)-1,3,2-dioxaborolane (1.04 g, 4.09 mmol, 1.30 eq.) and potassium acetate (1.55 g, 15.8 mmol, 5.00 eq.) in dimethyl sulfoxide (15 mL) was added Pd(dppf)Cl₂ (230 mg, 315 μμmol, 0.10 eq.) at 25° C. under nitrogen atmosphere. The solution was stirred at 80° C. for 12 hours under nitrogen atmosphere. The solution was diluted with water (20 mL), extracted with ethyl acetate (20 mL×3), washed with brine (15.0 mL×3), dried over anhydrous sodium sulfate, filtered and concentrated under reduced pressure and the residue was purified by column chromatography (silica gel, Ethyl acetate/Petroleum ether 1-20%) to get 6-(4,4,5,5-tetramethyl-1,3,2-dioxaborolan-2-yl)benzothiophene-3-carbonitrile (700 mg, 78% yield) as a white solid.

¹H NMR (400 MHz, CDCl₃) δ=8.40 (s, 1H), 8.19 (s, 1H), 8.02-8.00 (m, 1H), 7.96-7.94 (m, 1H), 1.39 (s, 12H).

Step 2: A solution of 6-(4,4,5,5-tetramethyl-1,3,2-dioxaborolan-2-yl)benzothiophene-3-carbonitrile (700 mg, 2.45 mmol, 1.00 eq.), dichlorocopper (990 mg, 7.36 mmol, 3.00 eq.) and 1-chloropyrrolidine-2,5-dione (361 mg, 2.70 mmol, 1.10 eq.) in methanol (10.0 mL) and water (10.0 mL) was stirred at 80° C. for 12 hours. The mixture was concentrated under reduced pressure to remove methanol, diluted with water (30 mL×3) and extracted with ethyl acetate (30 mL×3). The organic layers were washed with brine (8 mL×3), dried over anhydrous sodium sulfate, filtered and concentrated under reduced pressure and the residue was purified by column chromatography (silica gel, Ethyl acetate/Petroleum ether 0-5%) to get 6-chlorobenzo[b]thiophene-3-carbonitrile (580 mg, crude) as a white solid. ¹H NMR (400 MHz, CDCl₃) δ=8.13 (s, 1H), 7.94-7.91 (m, 2H), 7.55-7.51 (m, 1H).

Step 3: To a solution of 6-chlorobenzo[b]thiophene-3-carbonitrile (380 mg, 1.96 mmol, 1.00 eq.) in 2-methyl THF (5.0 mL) purged with nitrogen for 3 times at −70° C. was added lithium diisopropylamine (2.00 M, 1.18 mL, 1.20 eq.) dropwise. After stirring for 1 hour, iodine (996 mg, 3.92 mmol, 791 μL, 2.00 eq.) in 2-methyl THF (10.0 mL) was added to the solution. The resulting mixture was stirred at 25° C. for 2 hours under nitrogen. The reaction mixture was quenched by addition with saturated sodium sulfite (10 mL) at 0° C., then diluted with water (10 mL) and extracted with ethyl acetate (20 mL×3). The combined organic layers were washed with brine (15.0 mL×3), dried over anhydrous sodium sulfate, filtered and concentrated under reduced pressure and the solution was purified by prep-TLC (silica gel plate, Petroleum ether/Ethyl acetate 10:1) to get 6-chloro-2-iodobenzo[b]thiophene-3-carbonitrile (250 mg, crude) as a yellow solid. ¹H NMR (400 MHz, CDCl₃) δ=7.84 (d, J=8.4 Hz, 1H), 7.81 (d, J=1.6 Hz, 1H), 7.46 (dd, J=8.4, 2.0 Hz, 1H).

Step 4: To a solution of 6-chloro-2-iodobenzo[b]thiophene-3-carbonitrile (162 mg, 782 μmol, 1.00 eq.) and 6-chloro-2-iodo-benzothiophene-3-carbonitrile (250 mg, 782 μmol, 1.00 eq.) in 1,4-dioxane (4.5 mL) and water (1.5 mL) was added [2-(2-aminophenyl)phenyl]palladium(1+);bis(1-adamantyl)-butyl-phosphane;methanesulfonate (60.0 mg, 78.2 μμmol, 0.10 eq.) and sodium bicarbonate (197 mg, 2.35 mmol, 91.3 μL, 3.00 eq.) at 25° C. under nitrogen atmosphere. The solution was stirred at 65° C. for 2 hours under nitrogen atmosphere. The solution was concentrated under reduced and the residue was purified by prep-TLC (silica gel plate, Petroleum ether/Ethyl acetate 2:1) to give 6-chloro-2-(1-methyl-1H-pyrazol-5-yl)benzo[b]thiophene-3-carbonitrile (110 mg, 42% yield) as a yellow solid. ¹H NMR (400 MHz, CDCl₃) δ=7.94 (d, J=8.8 Hz, 1H), 7.90 (d, J=2.0 Hz, 1H), 7.63 (d, J=2.0 Hz, 1H), 7.57 (dd, J=10.4, 2.0 Hz, 1H), 6.79 (d, J=2.0 Hz, 1H), 4.09 (s, 3H).

Step 5: A solution of 6-chloro-2-(1-methyl-1H-pyrazol-5-yl)benzo[b]thiophene-3-carbonitrile (110 mg, 330 μμmol, 82% purity, 1.00 eq.), N-iodosuccinimide (222 mg, 989 μμmol, 3.00 eq.) and 4-methylbenzenesulfonic acid hydrate (6.27 mg, 33.0 μμmol, 0.10 eq.) in N,N-dimethylformamide (1.0 mL) and acetonitrile (1.0 mL) was stirred at 90° C. for 2 hours. The solution was quenched by addition with saturated sodium sulfite (2.0 mL) at 25° C., extracted with ethyl acetate (5.0 mL×3), washed with brine (2.0 mL 3), dried over anhydrous sodium sulfate, filtered and concentrated under reduced pressure and the residue was purified by prep-TLC (silica gel plate, Petroleum ether/Ethyl acetate 2:1) to give 6-chloro-2-(4-iodo-1-methyl-1H-pyrazol-5-yl)benzo[b]thiophene-3-carbonitrile (85 mg, 65% yield) as a white solid. ¹H NMR (400 MHz, CDCl₃) δ=7.99 (d, J=8.8 Hz, 1H), 7.95 (d, J=2.0 Hz, 1H), 7.69 (s, 1H), 7.60 (dd, J=8.8, 2.0 Hz, 1H), 3.99 (s, 3H).

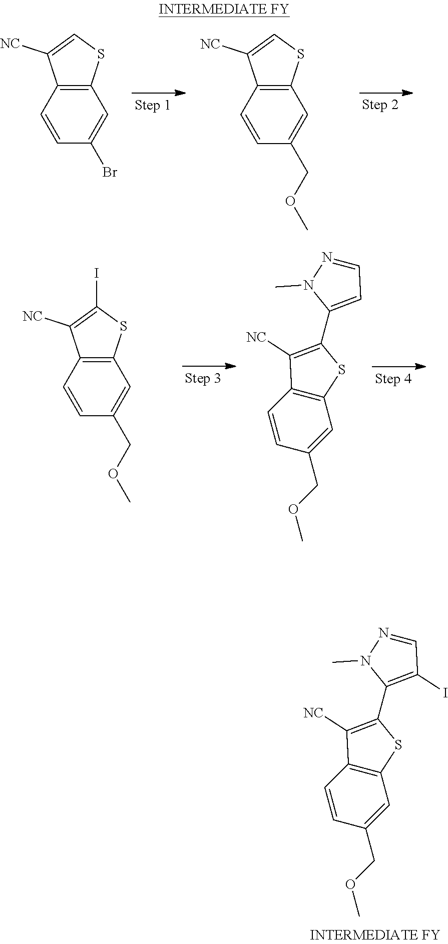

Step 1: To a solution of 6-bromobenzo[b]thiophene-3-carbonitrile (391 mg, 1.64 mmol, 1.00 eq.) and tributyl(methoxymethyl)stannane (550 mg, 1.64 mmol, 1.00 eq.) in 1,4-dioxane (10.0 mL) was added tetrakis[triphenylphosphine]palladium(0) (190 mg, 164 μμmol, 0.10 eq.). The mixture was stirred at 100° C. for 1 hour. The reaction mixture was quenched by addition saturated potassium fluoride solution (20 mL) and then extracted with ethyl acetate (20 mL×3). The combined organic layers were washed with brine (20 mL×2), dried over anhydrous sodium sulfate, filtered and concentrated under reduced pressure and the residue was purified by flash silica gel chromatography (Ethyl acetate/Petroleum ether 0-30%) to give 6-(methoxymethyl)benzo[b]thiophene-3-carbonitrile (120 mg, 36% yield) as a yellow solid. 105701 ¹H NMR (400 MHz, CDCl₃): δ=8.03 (s, 1H), 7.89 (d, J=8.4 Hz, 1H), 7.83 (d, J=0.4 Hz, 1H), 7.42 (dd, J=8.4, 1.2 Hz, 1H), 4.54 (s, 2H), 3.37 (s, 3H).

Step 2: To a solution of 6-(methoxymethyl)benzo[b]thiophene-3-carbonitrile (116 mg, 571 μμmol, 1.00 eq.) in 2-methyl THF (5.0 mL) purged with nitrogen for 3 times was added lithium diisopropylamide (2.00 M, 342 μL, 1.20 eq.) drop wise at −70° C. After addition, the mixture was stirred at this temperature for 1 hour, and then iodine (174 mg, 685 μmol, 138 μL, 1.20 eq.) in 2-methyl THF (5.0 mL) was added dropwise at −70° C. The resulting mixture was stirred at −70° C. for 3 hours. The reaction mixture was quenched by addition saturated sodium thiosulfate solution (10 mL) and extracted with ethyl acetate (10 mL×3). The combined organic layers were washed with brine (10 mL×3), dried over anhydrous sodium sulfate, filtered and concentrated under reduced pressure and the residue was purified by prep-TLC (silica gel plate, Petroleum ether/Ethyl acetate 10:1) to give the compound 2-iodo-6-(methoxymethyl)benzo[b]thiophene-3-carbonitrile (8.0 mg, 4% yield) as a yellow oil. ¹H NMR (400 MHz, CDCl₃): δ=7.86 (d, J=8.0 Hz, 1H), 7.47-7.44 (m, 1H), 7.29 (d, J=7.2 Hz, 1H), 4.74 (s, 2H), 3.43 (s, 3H).

Step 3: A mixture of 2-iodo-6-(methoxymethyl)benzo[b]thiophene-3-carbonitrile (15 mg, 45.6 μμmol, 1.00 eq.), 1-methyl-5-(4,4,5,5-tetramethyl-1,3,2-dioxaborolan-2-yl)-1H-pyrazole (11 mg, 54.7 μμmol, 1.20 eq), sodium bicarbonate (8 mg, 91.1 μμmol, 3.54 μL, 2.00 eq.) and bis(diphenylphosphino)ferrocene]dichloropalladium(II) (3.3 mg, 4.56 μμmol, 0.10 eq.) in 1,4-dioxane (1.0 mL) and water (0.2 mL) was degassed and stirred at 80° C. for 2 hours under nitrogen atmosphere. The reaction mixture was quenched by addition of water (5.0 mL), and then extracted with ethyl acetate (5.0 mL×3). The combined organic layers were washed with brine (5.0 mL×3), dried over anhydrous sodium sulfate, filtered and concentrated under reduced pressure and the residue was purified by prep-TLC (silica gel plate, Petroleum ether/Ethyl acetate 4:1) to give the compound 6-(methoxymethyl)-2-(1-methyl-1H-pyrazol-5-yl)benzo[b]thiophene-3-carbonitrile (20 mg, crude) as a yellow solid. ¹H NMR (400 MHz, CDCl₃): δ=7.97 (d, J=7.6 Hz, 1H), 7.63 (d, J=1.6 Hz, 1H), 7.56 (t, J=7.6 Hz, 1H), 7.42 (d, J=7.2 Hz, 1H), 6.78 (d, J=1.6 Hz, 1H), 4.80 (s, 2H), 4.10 (s, 3H), 3.46 (s, 3H).

Step 4: A mixture of 6-(methoxymethyl)-2-(1-methyl-1H-pyrazol-5-yl) benzo[b]thiophene-3-carbonitrile (20 mg, 70.6 μμmol, 1.00 eq.), N-iodosuccinimide (40 mg, 176.5 μμmol, 2.5. eq.) and 4-methylbenzenesulfonic acid hydrate (1.3 mg, 7.1 μμmol, 0.10 eq.) in N,N-dimethylformamide (0.50 mL) and acetonitrile (0.50 mL) was degassed and purged with nitrogen for 3 times, and then the mixture was stirred at 90° C. for 2 hours under nitrogen atmosphere. The reaction mixture was quenched by addition of saturated sodium thiosulfate solution (5.0 mL), and then extracted with ethyl acetate (5.0 mL×3). The combined organic layers were washed with brine (5.0 mL×3), dried over anhydrous sodium sulfate, filtered and concentrated under reduced pressure to give 2-(4-iodo-1-methyl-1H-pyrazol-5-yl)-6-(methoxymethyl)benzo[b]thiophene-3-carbonitrile (25 mg, crude) that was used in next step without further purification to give the compound as a yellow oil. LCMS [M+1]⁺: 410.2.

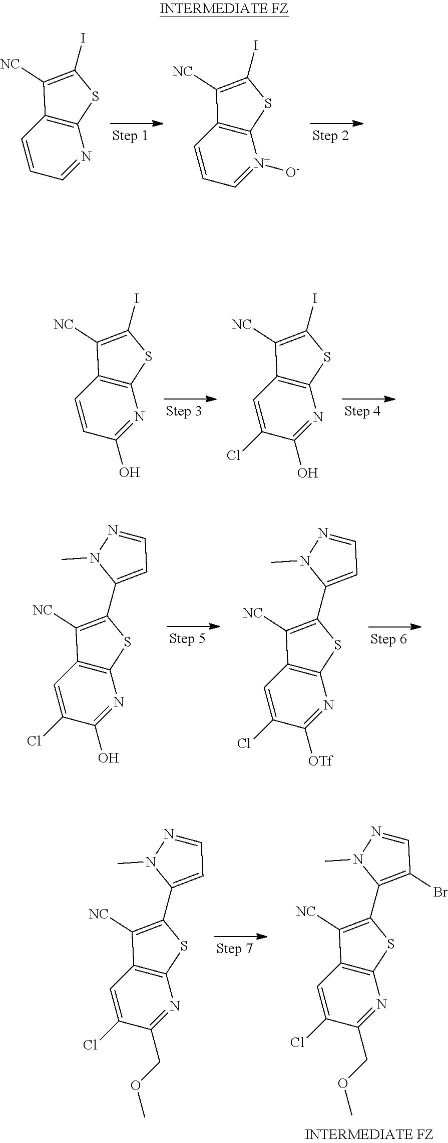

Step 1: To a solution of 2-iodothieno[2,3-b]pyridine-3-carbonitrile (3.00 g, 10.5 mmol, 1.00 eq.) in dichloromethane (80 mL) was added m-chloroperbenzoic acid (6.39 g, 31.5 mmol, 85% purity, 3.00 eq.). The mixture was stirred at 20° C. for 12 hours. During this period a white precipitate was formed. It was collected by filtration and dried under high vacuum to get 2-iodo-7-oxido-thieno [2, 3-b] pyridin-7-ium-3-carbonitrile (4.50 g, crude) as a yellow solid. ¹H NMR (400 MHz, DMSO-d₆) δ=8.49 (d, J=6.4 Hz, 1H), 7.73-7.66 (m, 1H), 7.57-7.50 (m, 1H).

Step 2: To a solution of 2-iodo-7-oxido-thieno [2,3-b]pyridin-7-ium-3-carbonitrile (4.50 g, 14.9 mmol, 1.00 eq.) in dimethylformamide (30 mL) was added trifluoroacetic anhydride (22.7 g, 108 mmol, 15 mL, 7.24 eq.). The mixture was stirred at 20° C. for 1 hour. The reaction mixture was diluted with water (20 mL) and very slowly added to saturated aqueous sodium bicarbonate (30 mL). During this period, white precipitate was formed. It was collected by filtration and dried under high vacuum to get 6-hydroxy-2-iodo-thieno [2,3-b]pyridine-3-carbonitrile (2.20 g, 46% yield,) as a white solid. ¹H NMR (400 MHz, DMSO-d₆) δ=11.94 (s, 1H), 8.02 (d, J=8.8 Hz, 1H), 6.74 (d, J=8.8 Hz, 1H).

Step 3: To a solution of 6-hydroxy-2-iodo-thieno [2,3-b]pyridine-3-carbonitrile (2.00 g, 6.62 mmol, 1.00 eq.) in dimethylformamide (20 mL) was added 1-chloropyrrolidine-2,5-dione (2.21 g, 16.5 mmol, 2.50 eq.). The mixture was stirred at 50° C. for 4 hours. The reaction mixture was diluted with water (50 mL). During this period, white precipitate was formed. It was collected by filtration and dried under high vacuum to get 5-chloro-6-hydroxy-2-iodo-thieno [2,3-b]pyridine-3-carbonitrile (2.40 g, 70% yield) as a white solid. ¹H NMR (400 MHz, DMSO-d₆) δ=12.93 (s, 1H), 8.20 (s, 1H).

Step 4: A mixture of 5-chloro-6-hydroxy-2-iodo-thieno[2,3-b]pyridine-3-carbonitrile (2.40 g, 4.64 mmol, 65% purity, 1.00 eq.), 1-methyl-5-(4,4,5,5-tetramethyl-1,3,2-dioxaborolan-2-yl)pyrazole (1.45 g, 6.95 mmol, 1.50 eq.), [1,1-bis(diphenylphosphino) ferrocene]dichloropalladium(II) (169 mg, 232 μμmol, 0.05 eq.), sodium bicarbonate (774 mg, 9.21 mmol, 2.00 eq.) and water (3.0 mL) in dioxane (30 mL) was degassed and stirred at 90° C. for 12 hours under nitrogen atmosphere. After being cooled to room temperature, the mixture was concentrated under vacuum. The residue was purified by flash silica gel chromatography (Ethyl acetate/Petroleum ether 0-90° %) to give 5-chloro-6-hydroxy-2-(2-methylpyrazol-3-yl) thieno [2,3-b]pyridine-3-carbonitrile (2.00 g, crude) as a brown solid. LCMS [M+H]⁺=291.8

Step 5: To a solution of 5-chloro-6-hydroxy-2-(2-methylpyrazol-3-yl)thieno[2,3-b]pyridine-3-carbonitrile (0.80 g, 2.75 mmol, 1.00 eq.) in dichloromethane (30 mL) was added N,N-diisopropylethylamine (589 mg, 4.57 mmol, 794 μL, 1.20 eq.) and trifluoromethanesulfonic anhydride (3.22 g, 11.40 mmol, 1.88 mL, 3 eq.) at 0° C. for 10 min. A temperature of the mixture was raised to room temperature and the mixture was stirred for 12 hours. The residue was diluted with dichloromethane (50 mL) and water (30 mL). The layers were separated, the aqueous phase was extracted with dichloromethane (3×30 mL). The organic layer was dried over anhydrous sodium sulfate, filtered and concentrated under vacuum. The residue was purified by column chromatography on silica gel (Petroleum ether/Ethyl acetate 5:1) to get [5-chloro-3-cyano-2-(2-methylpyrazol-3-yl)thieno [2,3-b]pyridin-6-yl] trifluoromethanesulfonate (300 mg, 26% yield) as a yellow solid. ¹H NMR (400 MHz, CDCl₃-d) 7=8.52 (s, 1H), 7.91 (s, 1H), 7.01 (s, 1H), 4.23 (s, 3H).

Step 6: A mixture of [5-chloro-3-cyano-2-(2-methylpyrazol-3-yl)thieno[2,3-b]pyridin-6-yl] trifluoromethanesulfonate (300 mg, 709 μμmol, 1.00 eq.), tributyl (methoxymethyl)stannane (285 mg, 852 μμmol, 1.20 eq.), lithium chloride (90 mg, 2.13 mmol, 43.6 μL, 3.00 eq.), bis(triphenylphosphine)palladium(II) chloride (50 mg, 70.9 μμmol, 0.10 eq.) in dioxane (5.0 mL) was degassed and stirred at 90° C. for 12 hours under nitrogen atmosphere. After being cooled to room temperature, the mixture was concentrated under vacuum. The residue was purified by column chromatography on silica gel (Petroleum ether/Ethyl acetate 3:1) to give 5-chloro-6-(methoxymethyl)-2-(2-methylpyrazol-3-yl)thieno [2,3-b]pyridine-3-carbonitrile (200 mg, 88% yield) as a yellow solid. ¹H NMR (400 MHz, CDCl₃) δ=8.25 (s, 1H), 7.64 (d, J=2.0 Hz, 1H), 6.85 (d, J=2.0 Hz, 1H), 4.85 (s, 2H), 4.10 (s, 3H), 3.58 (s, 3H).

Step 7: To a solution of 5-chloro-6-(methoxymethyl)-2-(2-methylpyrazol-3-yl) thieno[2,3-b]pyridine-3-carbonitrile (100 mg, 314 μμmol, 1.00 eq.) in acetonitrile (5.0 mL) was added N-bromosuccinimide (56 mg, 314 μμmol, 1.00 eq.). The mixture was stirred at 70° C. for 2 hours. After being cooled to room temperature, the mixture was concentrated under vacuum. The residue was purified by column chromatography on silica gel (Petroleum ether/Ethyl acetate 5:1) to give 2-(4-bromo-2-methyl-pyrazol-3-yl)-5-chloro-6-(methoxymethyl) thieno [2,3-b]pyridine-3-carbonitrile (50 mg, 111 μμmol, 35% yield, 88% purity) as a white solid. ¹H NMR (400 MHz, CDCl₃-d) δ=8.31 (s, 1H), 7.65 (s, 1H), 4.87 (s, 2H), 3.98 (s, 3H), 3.59 (s, 3H).

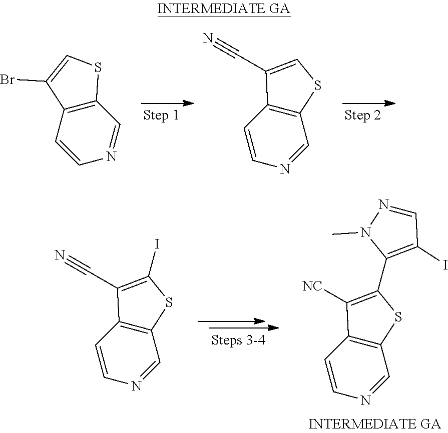 [SMILES: Brc1csc2cnccc12 ; N#Cc1csc2cnccc12 ; N#Cc1c(I)sc2cnccc12 ; Cn1ncc(I)c1-c1sc2cnccc2c1C#N]

Step 1: A mixture of zinc cyanide (197 mg, 1.68 mmol, 107 μL, 0.60 eq.), 3-bromothieno[2,3-c]pyridine (600 mg, 2.80 mmol, 1.00 eq.), tris-(dibenzylideneacetone)dipalladium(0) (513 mg, 561 μμmol, 0.20 eq.), 1,1′-bis(diphenylphosphino)ferrocene (155 mg, 280 μμmol, 0.10 eq.) and zinc (36.7 mg, 561 μμmol, 0.20 eq.) in dimethyl formamide (15 mL) was degassed and stirred at 120° C. for 2 hours under nitrogen atmosphere. The reaction mixture was diluted with water (30 mL) and extracted with ethyl acetate (20 mL×3). Combine organic layers were dried over sodium sulfate, concentrated in vacuo and the residue was purified by flash silica gel chromatography (Ethyl acetate/petroleum ether 0-50/6) to give thieno[2,3-c]pyridine-3-carbonitrile (290 mg, 64% yield) as a brown solid. ¹H NMR (400 MHz, DMSO-d₆) δ=9.46 (s, 1H), 9.20 (s, 1H), 8.66 (d, J=5.6 Hz, 1H), 7.93 (d, J=5.6 Hz, 1H).

Step 2: n-Butyl lithium (2.50 M, 1.09 mL, 1.50 eq.) was added drop wise to a solution of diisopropylamine (275 mg, 2.72 mmol, 384 μL, 1.50 eq.) in THF (15 mL) at −70° C. for 30 minutes. Then thieno[2,3-c]pyridine-3-carbonitrile (290 mg, 1.81 mmol, 1.00 eq.) in THF (5.0 mL) was added to the mixture. The reaction mixture was stirred for 1 hour at −70° C. before a solution of iodine (919 mg, 3.62 mmol, 729 μL, 2.00 eq.) in THF (10.0 mL) was added to the reaction mixture at −70° C. and the solution was stirred for 30 minutes at −70° C. and for 2 hours at 25° C. The mixture was quenched with water (60 mL) and extracted with ethyl acetate (50 mL 3). The combined organic extracts were washed brine (50 mL×3) and then dried over anhydrous sodium sulfate, filtered and concentrated in vacuo to give 2-iodothieno[2,3-c]pyridine-3-carbonitrile (520 mg, crude) as a brown solid. ¹H NMR (400 MHz, DMSO-d₆) δ=9.34 (s, 1H), 8.57 (d, J=5.6 Hz, 1H), 7.85 (d, J=5.6 Hz, 1H).

Step 3: A mixture of 2-iodothieno[2,3-c]pyridine-3-carbonitrile (520 mg, 1.82 mmol, 1.00 eq.), 1-methyl-5-(4,4,5,5-tetramethyl-1,3,2-dioxaborolan-2-yl)pyrazole (567 mg, 2.73 mmol, 1.50 eq.), Pd(dppf)Cl₂ (133 mg, 182 μμmol, 0.10 eq.) and sodium carbonate (578 mg, 5.45 mmol, 3.00 eq.) in dimethyl formamide (10.0 mL) and water (1.0 mL) was degassed and stirred at 90° C. for 14 hours. The reaction mixture was diluted with water (100 mL), extracted with ethyl acetate (300 mL). The combined organic layers were washed with brine (200 mL), dried over sodium sulfate, filtered and concentrated under reduced pressure and the residue was purified by flash silica gel chromatography (Ethyl acetate/Petroleum ether 0-50/6) to give 2-(2-methylpyrazol-3-yl)thieno[2,3-c]pyridine-3-carbonitrile (230 mg, 52% yield,) as a brown solid. ¹H NMR (400 MHz, DMSO-d₆) δ=9.50 (s, 1H), 8.72 (d, J=5.6 Hz, 1H), 7.96 (d, J=5.6 Hz, 1H), 7.72 (d, J=2.0 Hz, 1H), 6.96 (d, J=2.0 Hz, 1H), 4.06 (s, 3H).

Step 4: To a solution of 2-(2-methylpyrazol-3-yl)thieno[2,3-c]pyridine-3-carbonitrile (230 mg, 957 μμmol, 1.00 eq.) in acetonitrile (5.0 mL) and dimethyl formamide (5.0 mL) was added acetic acid (115 mg, 1.91 mmol, 109 μL, 2.00 eq.) and 1-iodopyrrolidine-2,5-dione (1.08 g, 4.79 mmol, 5.00 eq.). The mixture was stirred at 80° C. for 12 hours. The reaction mixture was quenched by addition of saturated ammonium chloride (10.0 mL) at 25° C., further diluted with water (5.0 mL) and extracted with ethyl acetate (30 mL). The combined organic layers were washed with brine (20 mL), dried over sodium sulfate, filtered and concentrated under reduced pressure to produce a residue. The residue was purified by prep-TLC (SiO₂, Dichloromethane/Methanol 15:1) to give 2-(4-iodo-2-methyl-pyrazol-3-yl)thieno[2,3-c]pyridine-3-carbonitrile (260 mg, 74% yield) as a yellow solid. ¹H NMR (400 MHz, DMSO-d₆) δ=9.56 (s, 1H), 8.75 (d, J=5.6 Hz, 1H), 8.03 (d, J=5.6 Hz, 1H), 7.86 (s, 1H), 3.94 (s, 3H).

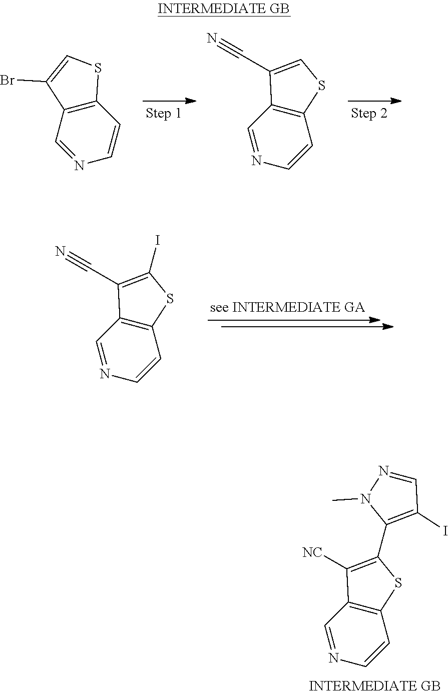 [SMILES: Brc1csc2ccncc12 ; N#Cc1csc2ccncc12 ; N#Cc1c(I)sc2ccncc12 ; Cn1ncc(I)c1-c1sc2ccncc2c1C#N]

Step 1: To a solution of 3-bromothieno[3,2-c]pyridine (1.00 g, 4.67 mmol, 1.00 eq.) in dimethyl formamide (30 mL) was added zinc cyanide (329 mg, 2.80 mmol, 0.60 eq.), 1,1′-Bis(diphenylphosphino)ferrocene (259 mg, 467 μμmol, 0.10 eq.), zinc (61 mg, 934 μμmol, 0.20 eq.) and tris-(dibenzylideneacetone)dipalladium(0) (855 mg, 934 μμmol, 0.20 eq.). The mixture was stirred at 120° C. for 2 hours. The reaction mixture was diluted with water (200 mL) and extracted with ethyl acetate (100 mL 3). The combined organic phase was washed with brine (200 mL), dried over anhydrous sodium sulfate, filtered and concentrated under vacuum to get a residue. The residue was purified by flash silica gel chromatography (Ethyl acetate/Petroleum 0-25%) to give thieno[3,2-c]pyridine-3-carbonitrile (600 mg, 80% yield) as a brown solid. ¹H NMR (400 MHz, DMSO-d₆) δ=9.21 (s, 1H), 9.00 (s, 1H), 8.61 (d, J=5.6 Hz, 1H), 8.28 (d, J=5.6 Hz, 1H).

Step 2: n-Butyl lithium (2.50 M, 1.12 mL, 1.50 eq.) was added to a solution of diisopropylamine (379 mg, 3.75 mmol, 529 μL, 2.00 eq.) in THF (9.00 mL) at −70° C. and the reaction mixture was stirred at −70° C. for 30 minutes. Thieno[3,2-c]pyridine-3-carbonitrile (300 mg, 1.87 mmol, 1.00 eq.) in THF (6.0 mL) was added to the mixture. The reaction mixture was stirred at −70° C. for 1 hour. Then a solution of iodine (713 mg, 2.81 mmol, 566 μL, 1.50 eq.) in THF (3.0 mL) was added to the reaction mixture at −70° C. and the solution was stirred for 30 minutes at −70° C. The reaction mixture was stirred at 25° C. for 2 hours. The reaction mixture was quenched by addition of saturated ammonium chloride (10 mL) at 25° C., further diluted with water (5.0 mL) and extracted with ethyl acetate (30 mL). The combined organic layers were washed with brine (20 mL), dried over anhydrous sodium sulfate, filtered and concentrated under reduced pressure to get 2-iodothieno[3,2-c]pyridine-3-carbonitrile (520 mg, crude) as a brown solid. ¹H NMR (400 MHz, DMSO-d₆) δ=9.09 (s, 1H), 8.54 (d, J=5.6 Hz, 1H), 8.19 (d, J=5.6 Hz, 1H).

Intermediate GB, 2-(4-iodo-2-methyl-pyrazol-3-yl)thieno[3,2-c]pyridine-3-carbonitrile was prepared as a yellow solid (390 mg, 59% yield over 2 steps) from 2-iodothieno[3,2-c]pyridine-3-carbonile following steps 3 and 4 of the procedure described for Intermediate GA. LCMS [M+1]⁺: 367.1. 1H NMR (400 MHz, DMSO-d₆) δ=9.31 (s, 1H), 8.71 (d, J=5.6 Hz, 1H), 8.37 (d, J=5.6 Hz, 11H), 7.85 (s, 1H), 3.94 (s, 3H).

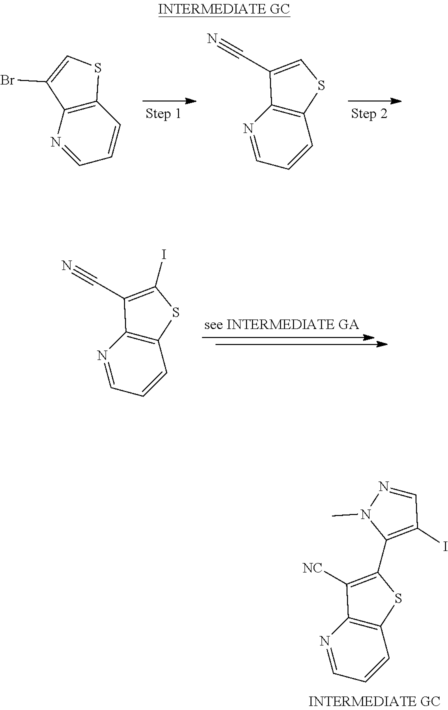 [SMILES: Brc1csc2cccnc12 ; N#Cc1csc2cccnc12 ; N#Cc1c(I)sc2cccnc12 ; Cn1ncc(I)c1-c1sc2cccnc2c1C#N]

Step 1: To a solution of 3-bromothieno[3,2-b]pyridine (50.0 mg, 234 μμmol, 1.00 eq.) in methylpyrrolidone (1.0 mL) was added copper cyanide (42 mg, 465 μμmol, 102 LL, 1.99 eq.). The mixture was stirred at 170° C. for 1 hour. The mixture was diluted with water (60 mL) and extracted with ethyl acetate (50 mL 3). The combined organic extracts were washed with brine (50 mL×3), dried over anhydrous sodium sulfate, filtered and concentrated in vacuum and the residue was purified by prep-TLC (SiO₂, Ethyl acetate/Petroleum ether 1:1) to give compound thieno[3,2-b]pyridine-3-carbonitrile (10 mg, 62.4 μμmol, 27% yield) as an orange solid. LCMS [M+1]⁺: 161.2. ¹H NMR (400 MHz, CDCl₃-d) □=8.88 (dd, J=1.2, 4.4 Hz, 1H), 8.41 (s, 1H), 8.27 (dd, J=1.2, 8.4 Hz, 1H), 7.43 (dd, J=4.8, 8.4 Hz, 1H).

Step 2: n-Butyllithium (2.50 M, 375 μL, 1.50 eq.) was added to a solution of N-isopropylpropan-2-amine (95 mg, 936 μμmol, 132 μL, 1.50 eq.) in THFTHF (3.0 mL) at −70° C. and the reaction mixture was stirred at −70° C. for 30 minutes. Then thieno[3,2-b]pyridine-3-carbonitrile (100 mg, 624 μμmol, 1.00 eq.) in THF (2.0 mL) was added to the mixture. And the reaction mixture was stirred for 1 hour at −70° C. Then a solution of iodine (317 mg, 1.25 mmol, 252 μL, 2.00 eq.) in THF (1.0 mL) was added to the reaction mixture at −70° C. and the solution was stirred for 30 minutes at −70° C. and for 2 hours at 25° C. The mixture was diluted with water (60 mL) and extracted with ethyl acetate (80 mL×3). The combined organic extracts were washed brine (50 mL×3), dried over anhydrous sodium sulfate, filtered and concentrated in vacuum and the residue was purified by prep-TLC (SiO₂, Petroleum ether/Ethyl acetate 3:1) to give compound 2-iodothieno[3,2-b]pyridine-3-carbonitrile (138 mg, 482 μμmol, 77% yield) as an orange solid. LCMS [M+1]⁺286.9. ¹H NMR (400 MHz, CDCl₃-d) δ=8.76 (d, J=4.8 Hz, 1H), 8.14 (dd, J=1.2, 8.4 Hz, 1H), 7.35 (dd, J=4.8, 8.4 Hz, 1H)

Intermediate GC, 2-(4-iodo-1-methyl-1H-pyrazol-5-yl)thieno[3,2-b]pyridine-3-carbonitrile was prepared as a yellow solid (76 mg, 46% yield over 2 steps) from 2-iodothieno[3,2-b]pyridine-3-carbonitrile following steps 3 and 4 of the procedure described for Intermediate GA. LCMS [M+1]⁺=366.9. ¹H NMR (400 MHz, CDCl₃-d) □=8.92 (dd, J=1.2, 4.8 Hz, 1H), 8.29 (dd, J=1.2, 8.4 Hz, 1H), 8.01 (s, 1H), 7.50 (dd, J=4.8, 8.4 Hz, 1H), 4.01 (s, 3H).

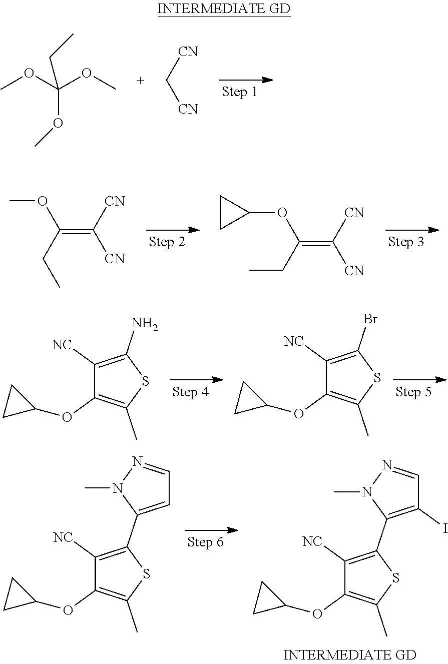

Step 1: A mixture of 1,1,1-trimethoxypropane (20.0 g, 149 mmol, 21.2 mL, 1.00 eq.) and malononitrile (9.85 g, 149 mmol, 9.38 mL, 1.00 eq.) was stirred at 60° C. for 3 hours. The mixture was concentrated. The residue was purified by flash silica gel chromatography (Ethyl acetate/petroleum ether 10-33%) to afford 2-(1-methoxypropylidene)malononitrile (19.0 g, 94% yield) as light-yellow oil. ¹H NMR (400 MHz, CDCl₃) δ=4.17 (s, 3H), 2.69 (q, J=7.6 Hz, 2H), 1.29 (t, J=7.6 Hz, 3H).

Step 2: To a mixture of 2-(1-methoxypropylidene)malononitrile (8.00 g, 58.8 mmol, 1.00 eq.) and cyclopropanol (4.10 g, 70.5 mmol, 1.20 eq.) was added potassium tert-butoxide (659 mg, 5.88 mmol, 0.10 eq.). The mixture was stirred at 100° C. for 16 hours. The mixture was concentrated. The residue was purified by flash silica gel chromatography (Ethyl acetate/petroleum ether 10-33%) to afford 2-(1-cyclopropoxypropylidene) malononitrile (4.00 g, 42% yield) as a light-yellow oil. ¹H NMR (400 MHz, CDCl₃) δ=4.27 (tt, J=3.2, 6.0 Hz, 1H), 2.73 (q, J=7.6 Hz, 2H), 1.29 (t, J=7.6 Hz, 3H), 1.03-0.92 (m, 4H).

Step 3: A mixture of 2-(1-cyclopropoxypropylidene)malononitrile (4.00 g, 24.7 mmol, 1.00 eq.) and sulphur (791 mg, 24.7 mmol, 1.00 eq.) in THF (60 mL) was stirred at 25° C. for 15 min. Then triethylamine (2.50 g, 24.7 mmol, 3.43 mL, 1.00 eq.) was added drop wise at 25° C. The mixture was stirred for 60° C. for 2 hours. The mixture diluted with water (200 mL) and extracted with ethyl acetate (50 mL×2). The organic layer was washed with brine (100 mL), dried over anhydrous sodium sulfate, filtered and concentrated. The residue was purified by flash silica gel chromatography (Ethyl acetate/petroleum ether 0-33%) to afford 2-amino-4-cyclopropoxy-5-methylthiophene-3-carbonitrile (2.50 g, 52% yield) as a light-yellow oil. ¹H NMR (400 MHz, CDCl₃) δ=4.77-4.41 (m, 2H), 4.07 (t, J=2.8, 6.0 Hz, 1H), 2.12 (s, 3H), 1.03-0.94 (m, 2H), 0.88-0.80 (m, 2H).

Step 4: To a mixture of copper(II) bromide (2.76 g, 12.4 mmol, 579 μL, 1.20 eq.) in acetonitrile (40.0 mL) was slowly added tert-butyl nitrite (1.59 g, 15.4 mmol, 1.84 mL, 1.50 eq.) at 0° C. The mixture was stirred at 0° C. for 15 min. A solution of 2-amino-4-cyclopropoxy-5-methylthiophene-3-carbonitrile (2.00 g, 10.3 mmol, 1.00 eq.) in acetonitrile (20 mL) was added to the above reaction mixture at 0° C. and stirred for 1 hour. The mixture was stirred at 20° C. for 5 hours. The mixture diluted with water (200 mL) and extracted with ethyl acetate (80 mL×3). The organic layer was washed with brine (200 mL), dried over anhydrous sodium sulfate, filtered and concentrated. The residue was purified by flash silica gel chromatography (Ethyl acetate/petroleum ether 0-33%) to afford 2-bromo-4-cyclopropoxy-5-methylthiophene-3-carbonitrile (1.10 g, 24.8% yield, 60% purity) as a light-yellow oil. LCMS [M+1]⁺: 259.9. ¹H NMR (400 MHz, CDCl₃) δ=4.16 (tt, J=2.8, 6.0 Hz, 1H), 2.26 (s, 3H), 0.92-0.85 (m, 2H), 0.74-0.67 (m, 2H).

Step 5: To a mixture of 2-bromo-4-cyclopropoxy-5-methylthiophene-3-carbonitrile (600 mg, 2.32 mmol, 1.00 eq.) and 1-methyl-5-(4,4,5,5-tetramethyl-1,3,2-dioxaborolan-2-yl)-1H-pyrazole (580 mg, 2.79 mmol, 1.20 eq.) in dioxane (12.0 mL) and water (2.5 mL) was added potassium carbonate (803 mg, 5.81 mmol, 2.50 eq.) and [1,1′-bis(diphenylphosphino)ferrocene]dichloropalladium(II) (170 mg, 232 μμmol, 0.10 eq.). The mixture was stirred at 80° C. for 16 hours under nitrogen atmosphere. The mixture was diluted with water (50 mL) and extracted with ethyl acetate (20 mL×3). The organic layer was washed with brine (50 mL), dried over anhydrous sodium sulfate, filtered and concentrated. The residue was purified by flash silica gel chromatography (Ethyl acetate/petroleum ether 0-50%) to afford 4-cyclopropoxy-5-methyl-2-(1-methyl-1H-pyrazol-5-yl)thiophene-3-carbonitrile (200 mg, 33% yield) as light yellow oil. ¹H NMR (400 MHz, CDCl₃) δ=7.55 (d, J=2.0 Hz, 1H), 6.60 (d, J=2.0 Hz, 1H), 4.22 (tt, J=2.8, 6.0 Hz, 1H), 3.99 (s, 3H), 2.38 (s, 3H), 0.97-0.90 (m, 2H), 0.77-0.69 (m, 2H).

Step 6: To a solution of 4-cyclopropoxy-5-methyl-2-(1-methyl-1H-pyrazol-5-yl)thiophene-3-carbonitrile (200 mg, 771 μμmol, 1.00 eq.) in dimethylformamide (5.0 mL) was added N-iodosuccinimide (1.74 g, 7.71 mmol, 10.0 eq.) and acetic acid (4.63 mg, 77.1 μμmol, 4.41 μL, 0.10 eq.) at 25° C. The mixture was degassed and stirred at 80° C. for 2 hours. The mixture was diluted with water (50 mL) and extracted with ethyl acetate (20 mL×3). The organic layer was washed with brine (50 mL), dried over anhydrous sodium sulfate, filtered and concentrated. The residue was purified by flash silica gel chromatography (Ethyl acetate/petroleum ether 0-33%) to to afford 4-cyclopropoxy-2-(4-iodo-1-methyl-1H-pyrazol-5-yl)-5-methylthiophene-3-carbonitrile (80 mg, 22% yield, 80% purity) as light yellow oil. LCMS [M+1]⁺:286.0.

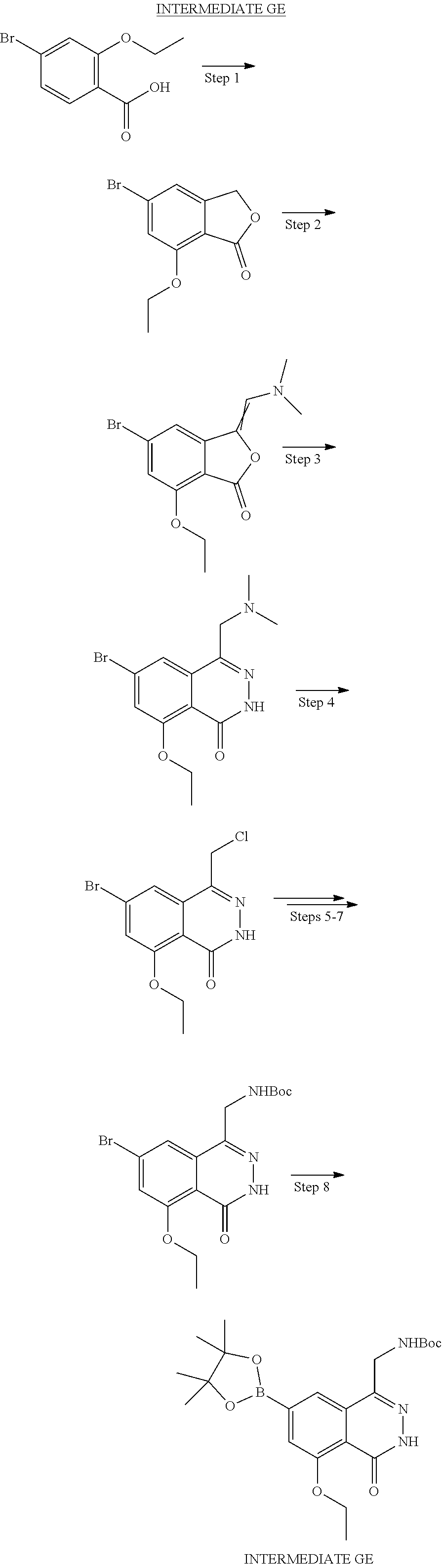

Step 1: Four reactions were carried out in parallel. To a solution of 4-bromo-2-ethoxybenzoic acid (50.0 g, 204 mmol, 1.00 eq) in dibromomethane (500 mL) was added Pd(OAc)₂ (4.58 g, 20.4 mmol, 0.10 eq) and K₂HPO₄ (107 g, 612 mmol, 3.00 eq). The mixture was stirred at 110° C. for 16 hrs. The mixture was cooled to room temperature. The four reaction mixture were combined, filtered and concentrated under reduced pressure and the residue was purified by column chromatography (SiO₂, Petroleum ether/Ethyl acetate 2-11%) to afford a product that was further triturated with Petroleum ether (500 mL, 1.8 V) at 25° C. for 15 mins, filtered and concentrated under reduced pressure to afford 5-bromo-7-ethoxyisobenzofuran-1 (3H)-one (140.5 g, 44% yield, 66% purity) as a light yellow solid. LCMS: M⁺ 259.0. ¹H NMR: (400 MHz, CDCl₃) δ 7.15 (s, 1H), 7.04 (s, 1H), 5.19 (s, 2H), 4.21 (q, J=6.8 Hz, 2H), 1.52 (t, J=7.2 Hz, 3H).

Step 2: To a solution of 5-bromo-7-ethoxyisobenzofuran-1 (3H)-one (150 g, 385 mmol, 66% purity, 1.00 eq) in DMF (1200 mL) was added t-BuOCH(NMe)₂ (101 g, 577 mmol, 119 mL, 1.50 eq). The mixture was stirred at 100° C. for 16 hrs. The reaction mixture was concentrated under reduced pressure to remove most of DMF. The reaction mixture was diluted with water (1000 mL) and EtOAc (800 mL). The mixture was filtered, the filter cake was collected, and the filter liquor extracted with EtOAc (300 mL×3). The combined organic layers were washed with brine (500 mL×3), dried over Na₂SO₄, filtered and concentrated under reduced pressure and the crude product was triturated with MTBE (200 mL, IV) at 25° C. for 30 mins, then filtered and concentrated under reduced pressure to afford 5-bromo-3-((dimethylamino)methylene)-7-ethoxyisobenzofuran-1 (3H)-one (97.0 g, 80/6 yield,) as a yellow solid. LCMS: (M)⁺ 312. ¹H NMR: (400 MHz, CDCl₃) δ7.00 (d, J=1.2 Hz, 1H), 6.62 (d, J=1.2 Hz, 1H), 6.10 (s, 1H), 4.17 (q, J=7.2 Hz, 2H), 3.12 (s, 6H), 1.49 (t, J=6.8 Hz, 3H).

Step 3: To a solution of 5-bromo-3-((dimethylamino)methylene)-7-ethoxyisobenzofuran-1 (3H)-one (100 g, 320 mmol, 1.00 eq) in EtOH (1500 mL) was added NH₂NH₂·H₂O (24.6 g, 481 mmol, 23.8 mL, 1.50 eq). The mixture was stirred at 80° C. for 30 hrs. The reaction mixture was filtered, and the filtrate concentrated under reduced pressure and the residue and filter cake were combined and triturated with EtOH (500 mL) at 20° C. for 30 mins. The mixture was filtered, and the filter cake was dried under reduced pressure to afford 6-bromo-4-((dimethylamino)methyl)-8-ethoxyphthalazin-1 (2H)-one (89.3 g, 85% yield) as a light yellow solid. LCMS: (M)⁺ 326.2. ¹H NMR: (400 MHz, DMSO-d₆) δ 12.3 (s, 1H), 7.81 (d, J=1.6 Hz, 1H), 7.46 (d, J=1.6 Hz, 1H), 4.18 (q, J=6.8 Hz, 2H), 3.52 (s, 2H), 2.16 (s, 6H), 1.37 (t, J=6.8 Hz, 3H).

Step 4: To a solution of 6-bromo-4-((dimethylamino)methyl)-8-ethoxyphthalazin-1 (2H)-one (90.0 g, 276 mmol, 1.00 eq) in THF (1350 mL) was added isobutyl carbonochloridate (56.5 g, 414 mmol, 54.4 mL, 1.50 eq) at 0° C. The mixture was stirred at 20° C. for 16 hrs before being concentrated under reduced pressure to remove THF. Then to the residue was added HCl (0.5M, 900 mL), the mixture was stirred at 20° C. for 30 mins. The mixture was filtered and the filter cake was washed with EtOAc (10 mL×3), and the solid was dried under reduced pressure to afford 6-bromo-4-(chloromethyl)-8-ethoxyphthalazin-1 (2H)-one (80.0 g, 75% yield) as a white solid. LCMS: (M+1)⁺ 318.9. ¹H NMR: (400 MHz, DMSO-d₆) δ 12.5 (s, 1H), 7.69 (d, J=1.2 Hz, 1H), 7.52 (d, J=1.6 Hz, 1H), 4.97 (s, 2H), 4.19 (q, J=6.8 Hz, 2H), 1.38 (t, J=6.8 Hz, 3H).

Step 5: To a solution of potassium phthalimide (57.1 g, 308 mmol, 1.50 eq) in DMF (800 mL) was added 6-bromo-4-(chloromethyl)-8-ethoxyphthalazin-1 (2H)-one (80.0 g, 206 mmol, 81.6% purity, 1.00 eq) at 0° C. The mixture was stirred at 20° C. for 2 hrs. To the mixture was added water (1000 mL), the resulting was stirred at 20° C. for 30 mins, then the mixture was filtered. The solid was dried under reduced pressure to give a crude product that was further triturated with EtOH (300 mL) at 20° C. for 30 mins. Then the mixture was filtered, the filter cake was dried under reduced pressure to afford 2-((7-bromo-5-ethoxy-4-oxo-3,4-dihydrophthalazin-1-yl)methyl)isoindoline-1,3-dione (86.0 g, 84% yield, 86% purity) as a white solid. LCMS: (M+1)+430.0.

Step 6: To a solution of 2-((7-bromo-5-ethoxy-4-oxo-3,4-dihydrophthalazin-1-yl)methyl)isoindoline-1,3-dione (102 g, 204 mmol, 86% purity, 1.00 eq) in EtOH (1500 mL) was added NH₂NH₂·H₂O (41.7 g, 817 mmol, 40.5 mL, 4.00 eq). The mixture was stirred at 75° C. for 4 hrs. The mixture was filtered, and the filter cake was washed with DCM/MeOH (10:1, 500 mL). The filtrate was concentrated under reduced pressure to afford a crude product that was triturated with EtOH (5V) at 25° C. for 30 mins. Then the mixture was filtered and the filter cake was dried under reduced pressure to afford 4-(aminomethyl)-6-bromo-8-ethoxyphthalazin-1 (2H)-one (86.0 g, 80% purity) as a white solid that was used in the next step directly. LCMS: (M+1)⁺ 300.2. ¹H NMR: (400 MHz, DMSO-d₆) δ 7.73 (d, J=1.2 Hz, 1H), 7.45 (d, J=1.6 Hz, 1H), 4.17 (q, J=7.2 Hz, 2H), 3.89 (s, 2H), 1.37 (t, J=6.8 Hz, 3H).

Step 7: To a solution of compound 4-(aminomethyl)-6-bromo-8-ethoxyphthalazin-1 (2H)-one (66.0 g, 221 mmol, 1.00 eq) and triethylamine (67.2 g, 664 mmol, 92.4 mL, 3.00 eq) in DCM (1650 mL) was added Boc₂O (96.6 g, 443 mmol, 102 mL, 2.00 eq). The mixture was stirred at 20° C. for 2 hrs before being filtered. The solid dried under reduced pressure to afford tert-butyl ((7-bromo-5-ethoxy-4-oxo-3,4-dihydrophthalazin-1-yl)methyl)carbamate (38.0 g) as a white solid that was used directly in the next step. ¹H NMR: (400 MHz, DMSO-d₆) δ 12.3 (s, 1H), 7.68 (s, 1H), 7.49 (d, J=1.2 Hz, 1H), 7.37 (t, J=5.6 Hz, 1H), 4.33 (d, J=5.6 Hz, 2H), 4.18 (q, J=6.8 Hz, 2H), 1.36-1.40 (m, 12H).

Step 8: A mixture of tert-butyl ((7-bromo-5-ethoxy-4-oxo-3,4-dihydrophthalazin-1-yl)methyl)carbamate (35.0 g, 87.8 mmol, 1.00 eq), Pd(dppf)Cl₂ (6.43 g, 8.78 mmol, 0.10 eq), KOAc (25.8 g, 263 mmol, 3.00 eq) and bis(pinacolato)diboron (33.4 g, 131.7 mmol, 1.50 eq) in dioxane (700 mL) was purged with N₂ for 3 times, and then the mixture was stirred at 100° C. for 5 hrs under N₂ atmosphere. The reaction mixture was filtered, and the filtrate was concentrated under reduced pressure and the crude product was triturated with Petroleum ether/Ethyl acetate 3:1 (200 mL) at 20° C. for 30 mins. The reaction mixture was filtered and the solid was dried under reduced pressure and the solid was triturated with MeOH (85 mL) at 20° C. for 30 mins. Then the mixture was filtered and the filter cake was dried under reduced pressure to afford tert-butyl ((5-ethoxy-4-oxo-7-(4,4,5,5-tetramethyl-1,3,2-dioxaborolan-2-yl)-3,4-dihydrophthalazin-1-yl)methyl)carbamate (28.5 g, 58.4 mmol, 69% yield, 91% purity) as a gray solid. LCMS: (M+1)⁺ 446.4. ¹H NMR: EW26347-38-P1A (400 MHz, DMSO-d₆) δ 12.2 (s, 1H), 7.71 (s, 1H), 7.42 (s, 1H), 7.25 (t, J=4.8 Hz, 1H), 4.39 (d, J=5.6 Hz, 2H), 4.16 (q, J=6.8 Hz, 2H), 1.37-1.41 (m, 12H), 1.32 (s, 12H).

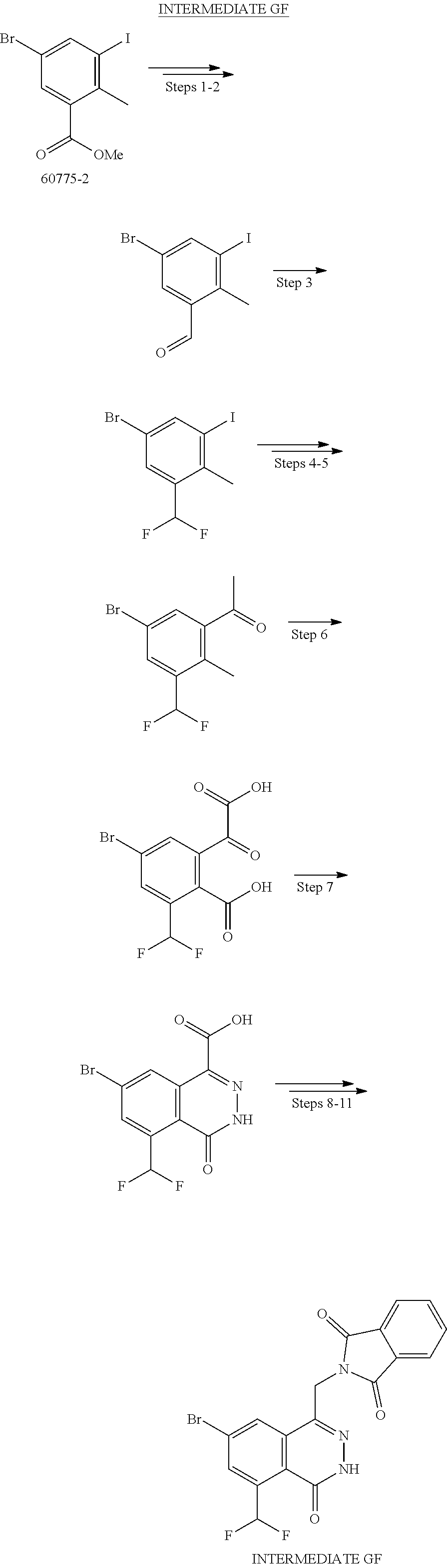

Step 1: To a solution of methyl 5-bromo-3-iodo-2-methyl-benzoate (8.50 g, 23.9 mmol, 1.00 eq.) in methylbenzene (150 mL) was added DIBAL (1 M, 36.0 mL, 1.50 eq.) at −70° C., the mixture was stirred at −70° C. for 0.5 h. The reaction was quenched with water (200 mL) and extracted with ethyl acetate (3×30 mL). The combined organic extracts were washed with saturated ammonium chloride (100 mL) and brine (100 mL), dried over sodium sulfate, filtered and concentrated to give (5-bromo-3-iodo-2-methyl-phenyl)methanol (7.10 g, 91% yield) as a yellow solid. ¹H NMR (400 MHz, (400 MHz, CDCl₃-d) δ ppm 2.36 (s, 3H), 4.67 (d, J=4.0 Hz, 2H), 7.52 (d, J=2.0 Hz, 1H), 7.91 (d, J=2.0 Hz, 1H)

Step 2: To a solution of (5-bromo-3-iodo-2-methyl-phenyl)methanol (3.50 g, 10.7 mmol, 1.00 eq.) in dichloromethane (70.0 mL) was added (1,1,1-Triacetoxy)-1,1-dihydro-1,2-benziodoxol-3 (1H)-one (6.81 g, 16.1 mmol, 5.0 mL, 1.50 eq.) at 20° C., the mixture was stirred at 20° C. for 2 hours. The mixture was diluted with dichloromethane (100 mL), washed with saturated aqueous sodium sulfite (50 mL×3), brine (30 mL), dried over sodium sulfate, filtered and concentrated to give the crude product. The residue was purified by flash silica gel chromatography (Petroleum ether/Ethyl acetate 0-2%) to give 5-bromo-3-iodo-2-methyl-benzaldehyde (3.00 g, 86.3% yield) as white solid. ¹H NMR (400 MHz, CDCl₃-d) δ=10.15 (s, 1H), 8.20 (d, J=2.0 Hz, 1H), 7.91 (d, J=2.0 Hz, 1H), 2.72 (s, 3H).

Step 3: To a solution of 5-bromo-3-iodo-2-methyl-benzaldehyde (6.00 g, 18.5 mmol, 1.00 eq.) in dichloromethane (60 mL) was added diethylaminosulfur trifluoride (8.93 g, 55.4 mmol, 7.32 mL, 3.00 eq.) dropwise at 0° C., the mixture was stirred at 0° C. for 1 hour and 20° C. for 2 h. The reaction was quenched with water (30 mL), extracted with dichloromethane (3-50 mL). The combined organic extracts were washed with brine (50 mL), dried over sodium sulfate, filtered and concentrated. The residue was purified by column chromatography (SiO₂, Petroleum ether) to give 5-bromo-1-(difluoromethyl)-3-iodo-2-methyl-benzene (6.60 g, crude) as a colorless oil. ¹H NMR (400 MHz, CDCl₃-d) δ=8.09 (s, 1H), 7.65 (d, J=1.6 Hz, 1H), 6.69 (t, J=55.2 Hz 1H), 2.46 (s, 3H).

Step 4: To a solution of 5-bromo-1-(difluoromethyl)-3-iodo-2-methyl-benzene (6.60 g, 19.0 mmol, 1.00 eq.), tributyl(1-ethoxyvinyl)stannane (7.30 g, 20.2 mmol, 6.82 mL, 1.06 eq.) in dioxane (10.0 mL) was added dichloropalladium triphenylphosphane (668 mg, 951 μμmol, 0.05 eq.). The mixture was purged with nitrogen for 3 min and then heated at 80° C. under nitrogen for 12 hours. The reaction mixture was diluted with ethyl acetate (100 mL) and very slowly added to saturated aqueous potassium fluoride (100 mL). The mixture was washed with potassium fluoride (3×50 mL). The organic layer was dried over sodium sulfate, filtered and concentrated under vacuum to give 5-bromo-1-(difluoromethyl)-3-(1-ethoxyvinyl)-2-methyl-benzene (5.54 g, crude) as a yellow oil. The crude product was used directly in the next step without further purification.

Step 5: To a solution of 5-bromo-1-(difluoromethyl)-3-(1-ethoxyvinyl)-2-methyl-benzene (5.54 g, 19.0 mmol, 1.00 eq.) in dioxane (5.0 mL) was added hydrochloric acid (1.00 M, 34.6 mL, 1.82 eq.). The mixture was stirred at 25° C. for 1 hour. The residue was diluted with ethyl acetate (50 mL) and water (50 mL). The layers were separated, and the aqueous phase was extracted with ethyl acetate (3×50 mL). The organic layer was dried over sodium sulfate, filtered and concentrated under vacuum to give 1-(5-bromo-3-(difluoromethyl)-2-methylphenyl)ethanone (4.00 g, 80% yield) as a colorless oil. ¹H NMR (400 MHz, CDCl₃-d) δ=7.78 (s, 1H), 7.74 (s, 1H), 6.78 (t, J=54.8 Hz, 1H), 2.57 (s, 3H), 2.41 (s, 3H).

Step 6: To a solution of 1-[5-bromo-3-(difluoromethyl)-2-methyl-phenyl] ethanone (1.40 g, 5.32 mmol, 1.00 eq.) in water (20 mL) was very slowly added potassium permanganate (5.90 g, 37.3 mmol, 7.02 eq.) and potassium carbonate (1.12 g, 8.10 mmol, 1.52 eq.). The mixture was stirred at 50° C. for 3 hours. Then ethanol (10 mL) was added and the resulting mixture was stirred at 25° C. for 10 minutes. The solid was filtered off, the filtrate was extracted with ethyl acetate (30 mL×2). The aqueous phase was adjusted to pH=2 with conc. hydrochloric acid (15 mL). Dichloromethane (30 mL×3) was added, and the organic layer was separated and concentrated to give 4-bromo-2-(difluoromethyl)-6-oxalo-benzoic acid (1.40 g, crude) as a white solid. LCMS [M+1]⁺=322.9

Step 7: To a solution of 4-bromo-2-(difluoromethyl)-6-oxalo-benzoic acid (1.40 g, 4.33 mmol, 1.00 eq.) in ethyl alcohol (10.0 mL) was added hydrazine hydrate (444 mg, 8.69 mmol, 431 μL, 2.01 eq.). The mixture stirred was at 78° C. for 12 hours. The reaction mixture was filtered. The solid was dried under reduced pressure to give 7-bromo-5-(difluoromethyl)-4-oxo-3H-phthalazine-1-carboxylic acid (500 mg, 35% yield) as a white solid. LCMS [M+1]⁺=319.0

¹H NMR (400 MHz, DMSO-d₆) δ ppm 8.99-8.88 (m, 1H), 8.23-7.95 (m, 2H).

Step 8: To a solution of 7-bromo-5-(difluoromethyl)-4-oxo-3H-phthalazine-1-carboxylic acid (500 mg, 1.52 mmol, 97% purity, 1.00 eq.) in methyl alcohol (10.0 mL) was added sulfuric acid (913 mg, 9.12 mmol, 496 μL, 6.00 eq.). The mixture was stirred at 65° C. for 4 hours. The reaction mixture was filtered. The solid was dried under reduced pressure to give methyl 7-bromo-5-(difluoromethyl)-4-oxo-3H-phthalazine-1-carboxylate (720 mg, crude) as a white solid. LCMS [M+1]⁺=334.8. ¹H NMR (400 MHz, DMSO-d₆) δ ppm 13.48 (s, 1H), 8.92 (s, 1H), 8.25 (s, 1H), 8.02 (t, J=54.8 Hz, 1H), 3.92 (s, 3H).

Step 9: To a solution of methyl 7-bromo-5-(difluoromethyl)-4-oxo-3H-phthalazine-1-carboxylate (1.30 g, 3.90 mmol, 1.00 eq.) in ethyl alcohol (20 mL) was added sodium borohydride (894 mg, 23.6 mmol, 6.05 eq.) and calcium chloride (520 mg, 4.68 mmol, 1.20 eq.). The mixture was stirred at 0° C. for 2 hours. The reaction mixture was concentrated under reduced pressure and the residue was diluted with water (5.0 mL) and adjusted pH=5.00 with 1N hydrochloric acid (10 mL). The solid was filtered and washed with water (10 mL×3). The solid was triturated with ethyl alcohol (10 mL) to give 6-bromo-8-(difluoromethyl)-4-(hydroxymethyl)-2H-phthalazin-1-one (600 mg, 48% yield) as a white solid. LCMS [M+1]⁺=304.9. ¹H NMR (400 MHz, DMSO-d₆) δ ppm 12.86 (s, 1H), 8.49 (s, 1H), 8.21 (s, 1H), 8.08 (t, J=54.8 Hz, 1H), 5.62 (s, 1H), 4.70 (s, 2H).

Step 10: To a solution of 6-bromo-8-(difluoromethyl)-4-(hydroxymethyl)-2H-phthalazin-1-one (600 mg, 1.97 mmol, 1.00 eq.) in thionyl chloride (24.6 g, 207 mmol, 15 mL, 105 eq.) was added dimethylformamide (14.4 mg, 196 μμmol, 15.1 μL, 0.10 eq.). The mixture was stirred at 25° C. for 1 hour. The reaction mixture was concentrated under reduced pressure to give 6-bromo-4-(chloromethyl)-8-(difluoromethyl)-2H-phthalazin-1-one (650 mg, crude) as a white solid. ¹H NMR (400 MHz, DMSO-d₆) δ ppm 13.11 (s, 1H), 8.50 (s, 1H), 8.26 (s, 1H), 8.06 (t, J=13.2 Hz, 1H), 5.11 (s, 2H).

Step 11: To a solution of 6-bromo-4-(chloromethyl)-8-(difluoromethyl)-2H-phthalazin-1-one (650 mg, 2.01 mmol, 1.00 eq.) in dimethylformamide (5.0 mL) was added isoindoline-1, 3-dione (443 mg, 3.01 mmol, 1.50 eq.). The mixture was stirred at 90° C. for 2 hours. The reaction mixture was cooled to 20° C. and poured into water (5.0 mL). The solid was filtered and triturated with ethyl alcohol (5.0 mL) at 20° C. for 30 min. The crude product was triturated with dimethylformamide (10 mL) at 25° C. for 60 min to give 2-[[7-bromo-5-(difluoromethyl)-4-oxo-3H-phthalazin-1-yl]methyl]isoindoline-1,3-dione (450 mg, 39% yield, 75% purity) as a white solid. LCMS [M+3]⁺=435.9. ¹H NMR (400 MHz, DMSO-d₆) δ ppm 12.97 (s, 1H), 8.63 (s, 1H), 8.26 (s, 1H), 8.06 (s, 1H), 7.98-7.87 (m, 4H), 5.22 (s, 2H).

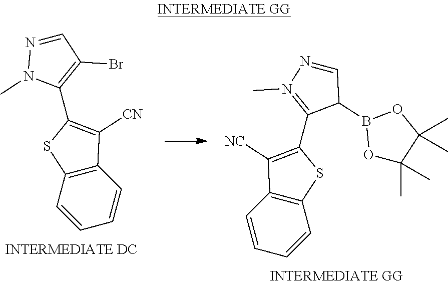

Intermediate GG was prepared from intermediate ES following the same procedure as described for intermediate DS starting from 2-(4-bromo-1-methyl-1H-pyrazol-5-yl)benzo[b]thiophene-3-carbonitrile (150 mg, 471 μμmol) to give 2-(1-methyl-4-(4,4,5,5-tetramethyl-1,3,2-dioxaborolan-2-yl)-1H-pyrazol-5-yl)benzo[b]thiophene-3-carbonitrile (170 mg, 223 μμmol) as a yellow oil. LCMS [M+1]⁺366.2

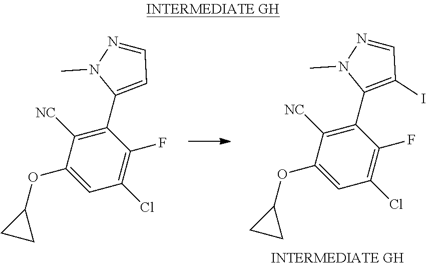 [SMILES: Cn1nccc1-c1c(F)c(Cl)cc(OC2CC2)c1C#N ; Cn1ncc(I)c1-c1c(F)c(Cl)cc(OC2CC2)c1C#N]

A mixture of 4-chloro-6-(cyclopropoxy)-3-fluoro-2-(2-methylpyrazol-3-yl)benzonitrile (5.60 g, 19.2 mmol, 1.00 eq.), N-iodosuccinimide (8.64 g, 38.4 mmol, 2.00 eq.) in acetic acid (60 mL) was degassed and stirred at 80° C. for 3 hours under nitrogen atmosphere. The reaction mixture was concentrated under reduced pressure and the residue was diluted with water (20 mL) and extracted with ethyl acetate (50 mL×3). Combined organic phase was washed with sat. aq. sodium bicarbonate (50 mL×2), brine (50 mL), dried over anhydrous sodium sulfate, filtered, and concentrated and the residue was purified by column chromatography (SiO₂, Petroleum ether/Ethyl acetate 5-20%) to give 4-chloro-6-(cyclopropoxy)-3-fluoro-2-(4-iodo-2-methyl-pyrazol-3-yl)benzonitrile (5.00 g, 62% yield) as a yellow solid. ¹H NMR (400 MHz, CDCl₃) δ=7.65 (s, 1H), 7.56 (d, J=6.0 Hz, 1H), 3.93-3.87 (m, 1H), 3.83 (s, 3H), 0.98-0.93 (m, 4H).

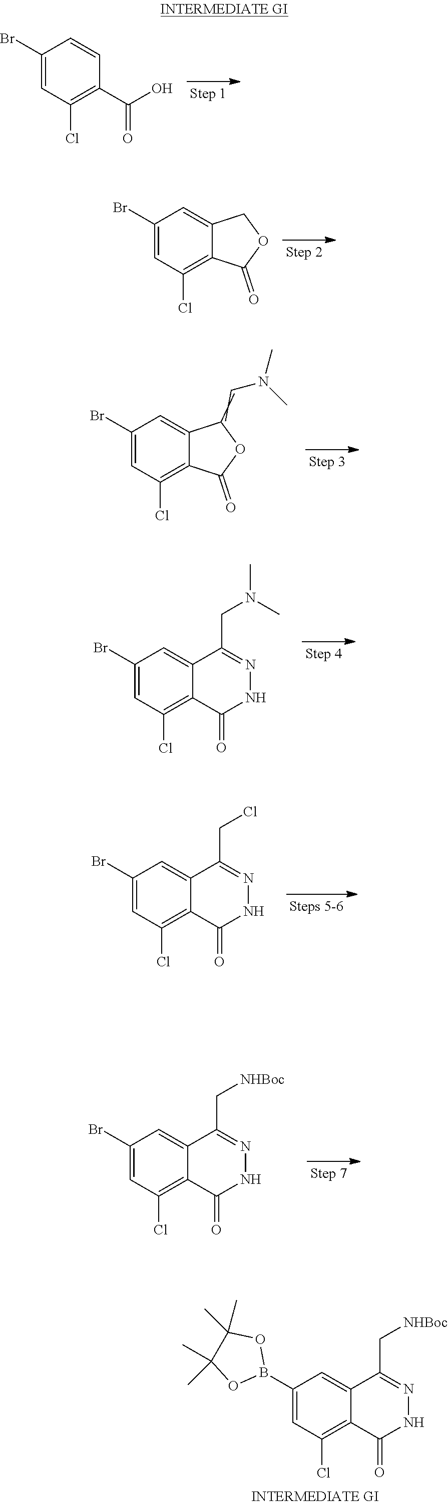

Step 1: To a solution of 4-bromo-2-chloro-benzoic acid (60.0 g, 255 mmol, 1.00 eq.) and dipotassium phosphate (133 g, 764 mmol, 3.00 eq.) in CH₂Br₂ (600 mL) was added a solution of Pd(OAc)₂ (11.4 g, 51.0 mmol, 0.20 eq.) at 20° C. in a high pressure autoclave. The reaction suspension was stirred at 130° C. for 48 hours. The solvent was removed under reduced pressure to give a residue which was then diluted with by water (500 mL) and extracted with ethyl acetate (300 mL×3). The combined organic phase was washed with brine (500 mL), dried over anhydrous sodium sulfate, filtered, and concentrated in vacuo and the residue was triturated with MTBE (50 mL) to give a yellow solid which was further purified by column chromatography (Petroleum ether/Ethyl acetate 5-33%) to give 5-bromo-7-chloro-3H-isobenzofuran-1-one (25.0 g, 40%) as a yellow solid. ¹H NMR (400 MHz, CHLOROFORM-d) δ=7.60 (s, 1H), 7.49 (s, 1H), 1.48 (s, 2H).

Step 2: A mixture of 5-bromo-7-chloroisobenzofuran-1 (3H)-one (15.0 g, 60.6 mmol, 1.00 eq.) and 1-tert-butoxy-N,N,N′,N′-tetramethylmethanediamine (12.7 g, 72.7 mmol, 15 mL, 1.2.0 eq.) in toluene (160 mL) was stirred at 105° C. for 3 hours. The mixture was concentrated. The residue was triturated with MTBE (50 mL). The mixture was filtered. The cake was dried under reduced pressure to afford 5-bromo-7-chloro-3-((dimethylamino)methylene)isobenzofuran-1 (3H)-one (15.6 g, 50% yield, 70% purity) as a yellow solid. LCMS [M+1]⁺: 303.9. ¹H NMR (400 MHz, DMSO-d₆) δ=7.93 (d, J=1.2 Hz, 1H), 7.33 (d, J=1.2 Hz, 1H), 7.12 (s, 1H), 3.13 (s, 6H).

Step 3: To a solution of 5-bromo-7-chloro-3-((dimethylamino)methylene)isobenzofuran-1 (3H)-one (19.0 g, 62.8 mmol, 1.00 eq.) in ethanol (300 mL) was added hydrazine hydrate (6.58 g, 129 mmol, 6.38 mL, 2.05 eq.). Then the mixture was stirred at 80° C. for 16 hours under nitrogen atmosphere. The mixture was concentrated to a volume of 150 mL. The mixture was filtered, and the filter cake was triturated with MTBE (100 mL). The mixture was filtered. The cake was dried under reduced pressure to afford 6-bromo-8-chloro-4-((dimethylamino)methyl)phthalazin-1 (2H)-one (16.0 g, 70% yield, 87% purity) as gray solid. LCMS [M+1]⁺: 317.9. ¹H NMR (400 MHz, DMSO-d₆) δ=12.79-12.45 (m, 1H), 8.30 (d, J=2.0 Hz, 1H), 8.10 (d, J=1.6 Hz, 1H), 3.58 (s, 2H), 2.18 (s, 6H).

Step 4: A mixture of 6-bromo-8-chloro-4-((dimethylamino)methyl)phthalazin-1 (2H)-one (16.0 g, 50.5 mmol, 1.00 eq.) in THF (180 mL) was purged with nitrogen for three times, cooled to 0° C. Isobutyl carbonochloridate (8.28 g, 60.7 mmol, 7.96 mL, 1.20 eq.) was added to the mixture drop-wise at the same temperature. The resulting mixture was stirred at 25° C. for 16 hours under nitrogen atmosphere. The mixture was filtered. The organic layer was collected. The filter cake was diluted with THF (150 mL). Then aq. hydrochloric acid (0.50 M, 150 mL) was added to the mixture and the mixture was filtered. The filter cake was washed with ethanol (50 mL) and petroleum ether (100 mL). The cake was dried under reduced pressure. The aqueous layer with aq. hydrochloric acid was adjusted to pH=8 with aq. saturated sodium bicarbonate. Then the mixture was filtered. The filter cake was washed with ethanol (50 mL) and dried under reduced pressure. The starting material was recycled. The collected organic layer was concentrated. The residue was triturated with Petroleum ether/Ethyl acetate (10:1, 50 mL). The mixture was filtered and concentrated under reduced pressure. The obtained solid from organic layer was combined with the above batch from filter cake. The solid was dried under reduced pressure to afford 6-bromo-8-chloro-4-(chloromethyl)phthalazin-1 (2H)-one (10.3 g, 60% yield) as gray solid. LCMS [M+1]⁺: 308.9. ¹H NMR (400 MHz, DMSO-d₆) δ=12.88 (s, 1H), 8.23 (d, J=1.6 Hz, 1H), 8.18 (d, J=1.6 Hz, 1H), 5.05 (s, 2H).

Step 5: To a solution of tert-butyl N-tert-butoxycarbonylcarbamate (9.19 g, 42.3 mmol, 1.05 eq.) in THF (250 mL) was added lithium bis(trimethylsilyl)amide (1.00 M, 44.3 mL, 1.10 eq.) at −30° C. under nitrogen. The mixture was stirred at 0° C. for 0.5 hour. Then a mixture of 6-bromo-8-chloro-4-(chloromethyl)phthalazin-1 (2H)-one (12.4 g, 40.3 mmol, 1.00 eq.) in THF (300 mL) was added to the above reaction mixture. The resulting reaction mixture was stirred at 20° C. for 2 hours. The mixture was quenched with aq. saturated ammonium chloride (1000 mL) and extracted with ethyl acetate (300 mL×2). The organic layer was washed with brine (600 mL), dried over sodium sulfate, filtered and concentrated. The crude material was triturated with ethyl acetate (300 mL). The mixture was filtered. The filter cake was washed with petroleum ether/ethyl acetate (5:1, 100 mL). The filtrate was concentrated. The residue was triturated with ethyl acetate (100 mL) and filtered again. The cake was washed with petroleum ether/ethyl acetate (5:1, 30 mL). Then the combined cake was dried under residue pressure to afford tert-butyl N-[(7-bromo-5-chloro-4-oxo-3H-phthalazin-1-yl)methyl]-N-tert-butoxycarbonyl-carbamate (16.0 g, 78% yield) as light yellow solid. LCMS [M+1]⁺: 490.0. ¹H NMR (400 MHz, DMSO-d₆) δ=12.68 (s, 1H), 8.21 (d, J=1.6 Hz, 1H), 8.16 (d, J=1.6 Hz, 1H), 4.99 (s, 2H), 1.39 (s, 18H).

Step 6: To a solution of tert-butyl N-[(7-bromo-5-chloro-4-oxo-3H-phthalazin-1-yl)methyl]-N-tert-butoxycarbonyl-carbamate (15.0 g, 30.7 mmol, 1.00 eq.) in acetonitrile (250 mL) was added magnesium diperchlorate hexahydrate (2.03 g, 6.14 mmol, 0.20 eq.). The mixture was stirred at 50° C. for 3 hours. The mixture was diluted with water (250 mL) and filtered. The filter cake was washed with water (100 mL), acetonitrile (100 mL) and petroleum ether (150 mL). The cake was dried under reduced pressure to afford tert-butyl ((7-bromo-5-chloro-4-oxo-3,4-dihydrophthalazin-1-yl)methyl)carbamate (11.5 g, 94% yield) as a white solid. LCMS [M+1]⁺: 390.0 ¹H NMR (400 MHz, DMSO-d₆) δ=12.67 (s, 1H), 8.18 (s, 1H), 8.13 (d, J=1.6 Hz, 1H), 7.43 (br t, J=5.6 Hz, 1H), 4.38 (br d, J=6.0 Hz, 2H), 1.40 (s, 9H).

Step 7: To a solution of tert-butyl ((7-bromo-5-chloro-4-oxo-3,4-dihydrophthalazin-1-yl)methyl)carbamate (6.00 g, 15.4 mmol, 1.00 eq.) and 4,4,4′,4′,5,5,5′,5′-octamethyl-2,2′-bi(1,3,2-dioxaborolane) (5.88 g, 23.2 mmol, 1.50 eq.) in dioxane (120 mL) was added potassium acetate (3.79 g, 38.6 mmol, 2.50 eq.) and [1,1′-bis(diphenylphosphino)ferrocene]dichloropalladium(II) (565 mg, 772 μμmol, 0.05 eq.). The mixture was stirred at 80° C. for 1 hour under nitrogen atmosphere. The mixture was filtered, and the filtrate was concentrated. The residue was triturated with methanol (80 mL) and filtered. The filter cake was washed with Petroleum ether/Ethyl acetate (50 mL, 5:1). The cake was dried under reduced pressure to afford tert-butyl ((5-chloro-4-oxo-7-(4,4,5,5-tetramethyl-1,3,2-dioxaborolan-2-yl)-3,4-dihydrophthalazin-1-yl)methyl)carbamate (5.00 g, 73% yield) as a white solid. LCMS [M+1]⁺: 354.1. ¹H NMR (400 MHz, DMSO-d₆) δ=12.63 (s, 1H), 8.13 (s, 1H), 7.91 (s, 1H), 7.34 (br t, J=5.2 Hz, 1H), 4.43 (br d, J=5.6 Hz, 2H), 1.41 (s, 9H), 1.32 (s, 12H).

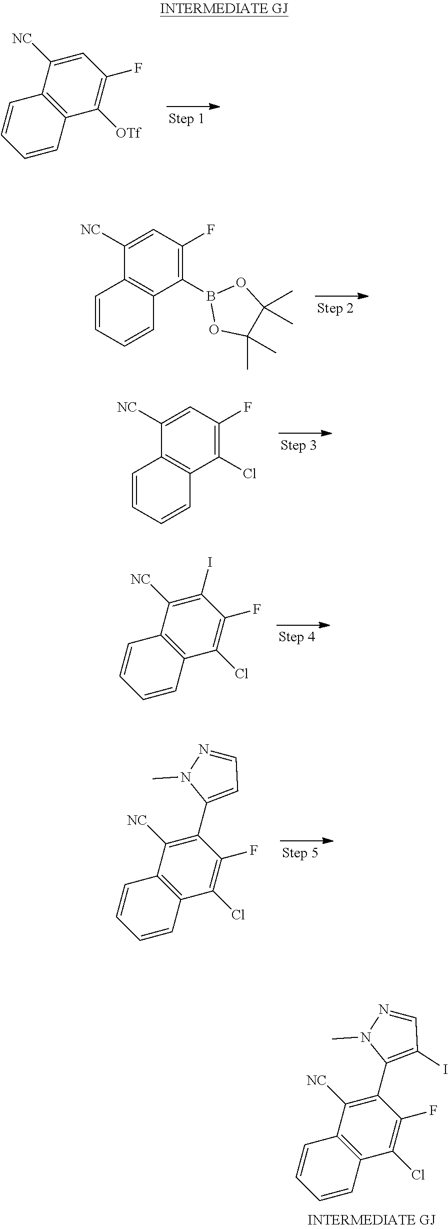

Step 1: A mixture of (4-cyano-2-fluoro-1-naphthyl)trifluoromethanesulfonate (500 mg, 1.57 mmol, 1.00 eq.), 4,4,5,5-tetramethyl-2-(4,4,5,5-tetramethyl-1,3,2-dioxaborolan-2-yl)-1,3,2-dioxaborolane (477 mg, 1.88 mmol, 1.20 eq.), potassium acetate (307 mg, 3.13 mmol, 2.00 eq.), Pd(dppf)Cl₂ (172 mg, 235 μμmol, 0.15 eq.) in dioxane (6.0 mL) was degassed and stirred at 100° C. for 2 hrs under nitrogen atmosphere. The reaction mixture was filtered and concentrated in vacuum. The residue was purified by column chromatography (SiO₂, Petroleum ether/Ethyl acetate 0-2%) to give 3-fluoro-4-(4,4,5,5-tetramethyl-1,3,2-dioxaborolan-2-yl)naphthalene-1-carbonitrile (450 mg, 97% yield) as a yellow solid.

Step 2: To a solution of 3-fluoro-4-(4,4,5,5-tetramethyl-1,3,2-dioxaborolan-2-yl)naphthalene-1-carbonitrile (450 mg, 1.51 mmol, 1.00 eq.) in methyl alcohol (10.0 mL) and water (10.0 mL) was added copper chloride (448 mg, 3.33 mmol, 2.20 eq.) and NCS (222 mg, 1.67 mmol, 1.10 eq.), the mixture was stirred at 80° C. for 5 hrs. The reaction mixture was filtered, the precipitate was dried in vacuum to give a crude product. The crude product was purified by column chromatography (SiO₂, Petroleum ether/Ethyl acetate 2-10%) to give 4-chloro-3-fluoro-naphthalene-1-carbonitrile (280 mg, 90% yield) as a light yellow solid. ¹H NMR (400 MHz, CDCl₃) δ=8.36 (d, J=8.0 Hz, 1H), 8.28 (d, J=7.6 Hz, 1H), 7.85-7.72 (m, 3H).

Step 3: To a solution of N-isopropylpropan-2-amine (251 mg, 2.48 mmol, 351 IL, 3.00 eq.) in THF (2.0 mL) was added n-butyllithium (2.5 M, 695 μL, 2.10 eq.) drop-wise at −65° C., stirring continue for 0.25 hr, then 4-chloro-3-fluoro-1-naphthonitrile (170 mg, 826.79 μmol, 1 eq.) in THF (1.0 mL) was added. After addition the mixture was stirred at −65° C. for 0.25 hr, before iodine (420 mg, 1.65 mmol, 333 μL, 2.00 eq.) was added and the resulting mixture was slowly warmed to 28° C. and stirred for 0.5 hour. The reaction mixture was quenched by addition of saturated sodium sulfite aqueous solution (10.0 mL) and extracted with ethyl acetate (15 mL×2), the combined organic phase was dried and concentrated in vacuum. The residue was purified by column chromatography (SiO₂, Petroleum ether/Ethyl acetate 1-10%) to give 4-chloro-3-fluoro-2-iodo-naphthalene-1-carbonitrile (200 mg, 73.0% yield) as a yellow solid.

Step 3: A mixture of 4-chloro-3-fluoro-2-iodo-naphthalene-1-carbonitrile (150 mg, 452 μmol, 1.00 eq.), 1-methyl-5-(4,4,5,5-tetramethyl-1,3,2-dioxaborolan-2-yl)pyrazole (122 mg, 588 μmol, 1.30 eq.), sodium bicarbonate (114 mg, 1.36 mmol, 52.8 LL, 3.00 eq.), Pd(dtbpf)Cl₂ (29.5 mg, 45.3 μmol, 0.10 eq.) in dioxane (2.0 mL) and water (0.4 mL) was degassed and mixture was stirred at 60° C. for 12 hrs under nitrogen atmosphere. The reaction mixture was filtered and concentrated in vacuum. The residue was purified by prep-TLC (SiO₂, Petroleum ether/Ethyl acetate 4:1) to give 4-chloro-3-fluoro-2-(2-methylpyrazol-3-yl)naphthalene-1-carbonitrile (100 mg, 77% yield) as an orange solid.

Step 5: To a solution of 4-chloro-3-fluoro-2-(2-methylpyrazol-3-yl)naphthalene-1-carbonitrile (100 mg, 350 μmol, 1.00 eq.) in acetic acid (5.0 mL) was added NIS (236 mg, 1.05 mmol, 3.00 eq.), the mixture was stirred at 30° C. for 12 hrs. The reaction mixture was adjusted to pH=7 with saturated sodium bicarbonate aqueous solution and extracted with ethyl acetate (5.0 mL 3), the combined organic phase was dried and concentrated. The residue was purified by column chromatography (SiO₂, Petroleum ether/Ethyl acetate 0-5%) to give 4-chloro-3-fluoro-2-(4-iodo-2-methyl-pyrazol-3-yl)naphthalene-1-carbonitrile (120 mg, 83% yield) as a yellow solid. LCMS [M+1]⁺: 412.0.

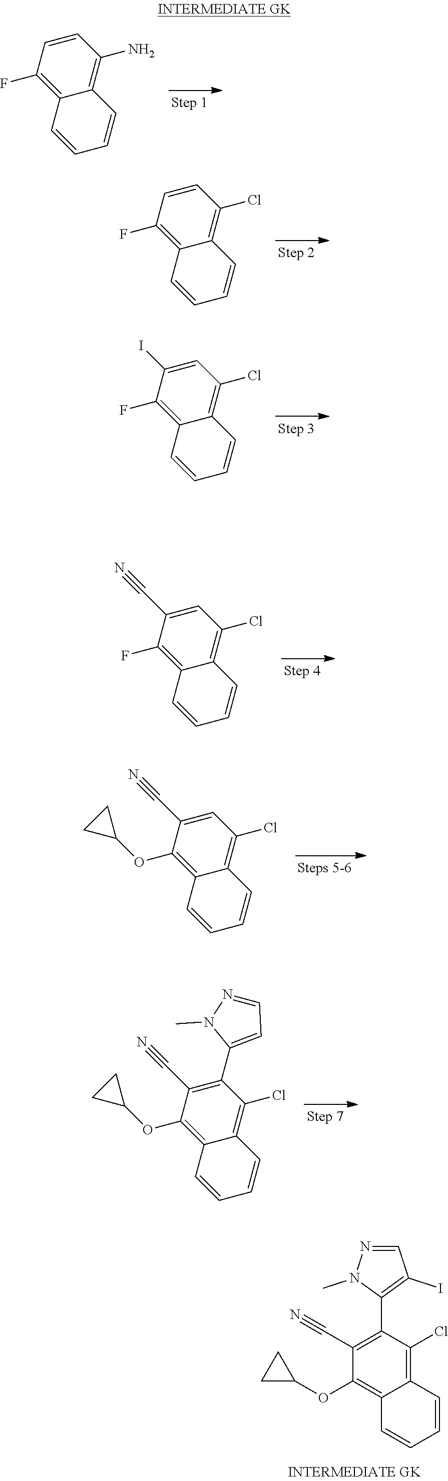

Step 1: To a stirred solution of 4-fluoronaphthalen-1-amine (1.65 g, 10.2 mmol, 1.00 eq.) in water (10.0 mL) was added hydrochloric acid (6.00 M, 33.0 mL, 19.3 eq.) followed by sodium nitrite (848 mg, 12.3 mmol, 1.20 eq.) at 0° C. (brine ice bath), and the stirring was continued for 2 hours below 5° C. The solution of cuprous chloride (4.05 g, 40.9 mmol, 979 μL, 4.00 eq.) in water (10.0 mL) was slowly added dropwise, the resulting solution was stirred for 14 hours at 25° C. The reaction was diluted with water (50 mL), extracted with ethyl acetate (50 mL×3), the combined organic phases were washed with saturated aqueous sodium bicarbonate (20 mL×3), then dried over anhydrous sodium sulfate, filtered and concentrated to produce a residue. The residue was purified by column chromatography (SiO₂, petroleum ether) to afford 1-chloro-4-fluoro-naphthalene (1.27 g, 69% yield) as a colorless oil. ¹H NMR (400 MHz, CDCl₃) δ=8.29-8.24 (m, 1H), 8.14 (dd, J=1.2, 8.8 Hz, 1H), 7.70-7.59 (m, 2H), 7.50 (dd, J=4.8, 8.4 Hz, 1H), 7.09 (dd, J=8.0, 10.0 Hz, 1H).

Step 2: To a solution of 1-chloro-4-fluoro-naphthalene (1.5 g, 8.31 mmol, 1.00 eq.) in THF (10 mL) was added n-butyllithium (2.50 M, 13.3 mL, 4.00 eq.) dropwise at −70° C. ad the resulting mixture was stirred at this temperature for 1 hour. Following that the solution of iodine (6.32 g, 24.9 mmol, 5.02 mL, 3 eq.) in THF (10.0 mL) was added slowly to at −70° C. The mixture was stirred at the same temperature for 30 min at 20° C. for 15 hours. The reaction mixture was quenched with ice water (10.0 mL) and saturated aqueous ammonium chloride (20 mL), the aqueous layer was extracted with ethyl acetate (15 mL×3), the combined organic phases were dried over anhydrous sodium sulfate, filtered and concentrated to deliver crude 4-chloro-1-fluoro-2-iodo-naphthalene (2.20 g) as a light-yellow solid that was used into next step without further purification. ¹H NMR (400 MHz, CDCl₃) δ=8.24-8.20 (m, 1H), 8.09 (dd, J=0.8, 8.4 Hz, 1H), 7.83 (d, J=5.2 Hz, 1H), 7.71-7.61 (m, 2H).

Step 3: A degassed mixture of 4-chloro-1-fluoro-2-iodo-naphthalene (2.20 g, 7.18 mmol, 1.00 eq.), zinc cyanide (506 mg, 4.31 mmol, 0.60 eq.), Pd(PPh₃)₄ (829 mg, 0.72 mmol, 0.10 eq.) in 1-methylpyrrolidin-2-one (22.0 mL) was stirred at 120° C. for 3 hours under nitrogen atmosphere. The reaction mixture was quenched with water (20 mL), the aqueous layer was extracted with ethyl acetate (10 mL×3). The combined organic phases were washed with brine (20 mL×3), dried over anhydrous sodium sulfate, filtered and concentrated to deliver a residue. The residue was purified by column chromatography (SiO₂, ethyl acetate/petroleum ether 0-5%) to give 4-chloro-1-fluoro-naphthalene-2-carbonitrile (1.00 g, 68% yield) as a white solid. ¹H NMR (400 MHz, CDCl₃) δ=8.31 (d, J=8.4 Hz, 1H), 8.23 (d, J=8.4 Hz, 1H), 7.86 (dt, J=1.6, 8.4 Hz, 1H), 7.79-7.74 (m, 1H), 7.63 (d, J=5.6 Hz, 1H)

Step 4: A solution of 4-chloro-1-fluoro-naphthalene-2-carbonitrile (1.05 g, 5.11 mmol, 1.00 eq.), cesium carbonate (3.33 g, 10.2 mmol, 2.00 eq.), cyclopropanol (593 mg, 10.2 mmol, 2.00 eq.) in N,N-dimethylformamide (20 mL) was stirred at 80° C. for 3 hours. The reaction was diluted with ethyl acetate (20 mL) and water (20 mL), then extracted with ethyl acetate (15 mL×3), the combined organic phase was washed with brine (15 mL×2), dried over anhydrous sodium sulfate, filtered and concentrated and the residue was purified by column chromatography (SiO₂, ethyl acetate/petroleum ether 0-5%) to give 4-chloro-1-cyclopropoxy-2-naphthonitrile (1.00 g, 80% yield) as a yellow solid. ¹H NMR (400 MHz, CDCl₃) δ=8.23 (t, J=7.6 Hz, 2H), 7.80-7.73 (m, 1H), 7.67-7.61 (m, 1H), 7.59 (s, 1H), 4.68-4.63 (m, 1H), 1.08-1.04 (m, 2H), 0.95-0.91 (m, 2H).

Step 5: To the cooled solution of diisopropylamine (623 mg, 6.16 mmol, 870 μL, 1.50 eq.) in 2-methylTHF (5.0 mL) at −70° C. was added n-butyllithium (2.5 M, 2.46 mL, 1.50 eq.) dropwise. After being stirred at −70° C. for 30 min, the mixture was treated with solution of 4-chloro-1-cyclopropoxy-2-naphthonitrile (1.00 g, 4.10 mmol, 1.00 eq.) in 2-methylTHF (10.0 mL). After 1 hour, the solution of iodine (1.56 g, 6.16 mmol, 1.24 mL, 1.50 eq.) in 2-methylTHF (5.0 mL) was added dropwise to the reaction, the mixture was stirred at −70° C. for 30 min, then warmed to 25° C. and stirred for 2 hours. The reaction was quenched with water (10.0 mL), diluted with ethyl acetate (10.0 mL), the aqueous layer was extracted with ethyl acetate (15 mL×3), washed with a solution of sodium thiosulfate (10 mL×2) and then washed with brine (10 mL). The organic phase was dried over anhydrous sodium sulfate, filtered and concentrated under reduced pressure and the residue was purified by column chromatography (SiO₂, petroleum ether/ethyl acetate 20:1) to give 4-chloro-1-cyclopropoxy-3-iodo-2-naphthonitrile (1.16 g, 77% yield) as a light-yellow solid. ¹H NMR (400 MHz, CDCl₃) δ=8.31 (d, J=8.4 Hz, 1H), 8.16 (d, J=8.4 Hz, 1H), 7.75 (dt, J=1.2, 6.8 Hz, 1H), 7.68-7.63 (m, 1H), 4.72-4.64 (m, 1H), 1.10-1.02 (m, 2H), 0.86-0.81 (m, 2H).

Step 6: A degassed mixture of 4-chloro-1-cyclopropoxy-3-iodo-2-naphthonitrile (200 mg, 541 μmol, 1.00 eq.), 1-methyl-5-(4,4,5,5-tetramethyl-1,3,2-dioxaborolan-2-yl)pyrazole (169 mg, 812 μmol, 1.50 eq.), (Ad₂n-BuP)—Pd G3 (40 mg, 54.1 μmol, 0.1 eq.) and sodium bicarbonate (90.9 mg, 1.08 mmol, 42.1 μL, 2.00 eq.) in dioxane (4.00 mL) and water (0.80 mL) was stirred at 80° C. under nitrogen for 12 hours. The reaction was concentrated directly and the residue was purified by column chromatography (SiO₂, petroleum ether/ethyl acetate 5:1) to give 4-chloro-1-cyclopropoxy-3-(1-methyl-1H-pyrazol-5-yl)-2-naphthonitrile (155 mg, 89% yield) as a white solid. LCMS [M+1]⁺=324.3. ¹H NMR (400 MHz, CDCl₃) δ=8.37 (d, J=8.4 Hz, 1H), 8.27 (d, J=8.4 Hz, 1H), 7.84 (td, J=1.2, 8.4 Hz, 1H), 7.73 (td, J=1.2, 8.4 Hz, 1H), 7.67 (d, J=2.0 Hz, 1H), 6.48 (d, J=2.0 Hz, 1H), 4.74-6.70 (m, 1H), 3.79 (s, 3H), 1.13-1.04 (m, 2H), 0.93-0.86 (m, 2H).

Step 7: To a solution of 4-chloro-1-(cyclopropoxy)-3-(2-methylpyrazol-3-yl) naphthalene-2-carbonitrile (100 mg, 309 μmol, 1.00 eq) in acetonitrile (2.0 mL) was added NBS (55.0 mg, 309 μmol, 1.00 eq). The mixture was stirred at 25° C. for 12 hours. The mixture was concentrated under vacuum. The residue was diluted with ethyl acetate (10.0 mL) and water (10.0 mL). The layers were separated, and the aqueous phase was extracted with ethyl acetate (3×10.0 mL). The organic layer was dried over sodium sulfate, filtered and concentrated under vacuum. The residue was purified by flash silica gel chromatography (SiO₂, ethyl acetate/petroleum ether 0-50%) to give 3-(4-bromo-2-methyl-pyrazol-3-yl)-4-chloro-1-(cyclopropoxy)naphthalene-2-carbonitrile (60.0 mg, 47% yield) as a yellow solid. ¹H NMR (400 MHz, CDCl₃) δ=8.38 (d, J=8.4 Hz, 1H), 8.30 (d, J=8.4 Hz, 1H), 7.88-7.81 (m, 1H), 7.78-7.71 (m, 1H), 7.66 (s, 1H), 4.73 (td, J=3.2, 6.0 Hz, 1H), 3.79 (s, 3H), 1.13-1.03 (m, 2H), 0.97-0.87 (m, 2H).

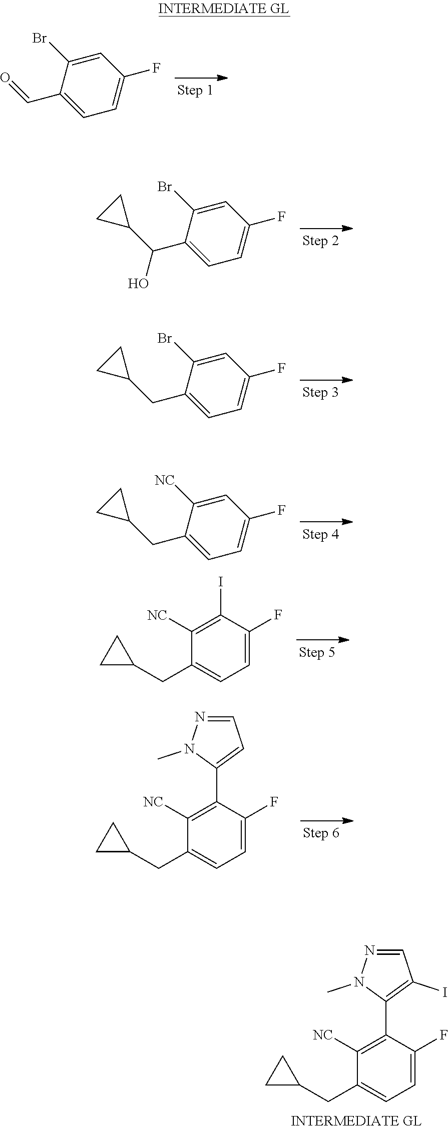

Step 1: To a mixture of 2-bromo-4-fluorobenzaldehyde (4.00 g, 19.7 mmol, 1.00 eq.) in 2-methyl THF (100 mL) was added bromo(cyclopropyl)magnesium (0.5 M, 39.4 mL, 1.00 eq.) dropwise at −78° C., the mixture was stirred at 20° C. for 3 hours under nitrogen atmosphere. After that, saturated ammonium chloride solution (100 mL) was added to the mixture and the mixture was stirred at 20° C. for 10 minutes. The mixture was extracted with ethyl acetate (100 mL×3), organic layers dried with anhydrous sodium sulfate, filtered and concentrated in vacuum and the residue was purified by column chromatography on silica gel (Petroleum ether/Ethyl acetate 1-33%) to give the compound (2-bromo-4-fluoro-phenyl)-cyclopropyl-methanol (4.50 g, 93% yield) as a colorless oil. ¹H NMR (400 MHz, CDCl₃): δ=7.52 (dd, J=8.8, 6.0 Hz, 1H), 7.29-7.13 (m, 1H), 6.99 (td, J=8.4, 2.8 Hz, 1H), 4.54 (d, J=7.2 Hz, 1H), 1.19-1.15 (m, 1H), 0.55-0.39 (m, 4H).

Step 2: To a solution of (2-bromo-4-fluoro-phenyl)-cyclopropyl-methanol (4.44 g, 18.1 mmol, 1.00 eq.) in dichloromethane (20 mL) was added triethylsilane (3.16 g, 27.2 mmol, 4.34 mL, 1.50 eq.) and trifluoroacetic acid (12.4 g, 109 mmol, 8.05 mL, 6.00 eq.) at 0° C., the resulting reaction mixture was stirred at 40° C. for 2 hours. The mixture was concentrated under reduced pressure and the residue was purified by flash silica gel chromatography (Petroleum ether/Ethyl acetate 0-20%) to give 2-bromo-1-(cyclopropylmethyl)-4-fluoro-benzene (1.80 g, 43% yield) as a colorless oil. ¹H NMR (400 MHz, CDCl₃): δ=7.38-7.34 (m, 1H), 7.30 (dd, J=8.4, 2.8 Hz, 1H), 7.00 (td, J=8.4, 2.4 Hz, 1H), 2.63 (d, J=7.2 Hz, 2H), 1.08-1.04 (m, 1H), 0.60-0.54 (m, 2H), 0.26-0.23 (m, 2H).

Step 3: A mixture of 2-bromo-1-(cyclopropylmethyl)-4-fluoro-benzene (500 mg, 2.18 mmol, 1.00 eq.), tetrakis[triphenylphosphine]palladium(0) (252 mg, 218 μmol, 0.10 eq.), zinc cyanide (384 mg, 3.27 mmol, 208 μL, 1.50 eq.) in N,N-Dimethylformamide (10.0 mL) was degassed and stirred at 110° C. for 2 hours under nitrogen atmosphere. The pH of mixture was adjusted pH to 11 by sodium hydroxide solution (1 N). The mixture was treated with sodium hypochlorite solution (50 mL) at 25° C., and then extracted with ethyl acetate (30 mL×3). The combined organic layers were washed with brine (30 mL×3), dried over anhydrous sodium sulfate, filtered and concentrated under reduced pressure and the residue was purified by flash silica gel (Petroleum ether/Ethyl acetate 0-10%) to give 2-(cyclopropylmethyl)-5-fluoro-benzonitrile (300 mg, 79% yield) as a yellow oil. ¹H NMR (400 MHz, CDCl₃): δ=7.22 (dd, J=8.4, 5.2 Hz, 1H), 7.10-7.02 (m, 2H), 2.53 (d, J=7.2 Hz, 2H), 0.83-0.78 (m, 1H), 0.38-0.32 (m, 2H), 0.07-0.04 (m, 2H).

Step 4: To a solution of 2-(cyclopropylmethyl)-5-fluoro-benzonitrile (300 mg, 1.71 mmol, 1.00 eq.) in degassed 2-methyl THF (12.0 mL) was added lithium diisopropylamine (2.00 M, 1.03 mL, 1.20 eq.) drop wise at −78° C. After addition, the mixture was stirred at this temperature for 40 minutes, and then iodine (522 mg, 2.05 mmol, 414 μL, 1.20 eq.) in 2-methyl THF (12.0 mL) was added dropwise at −78° C. The resulting mixture was stirred at 25° C. for 12 hours. The reaction mixture was quenched by addition saturated ammonium chloride solution (20 mL) at 25° C., and then extracted with ethyl acetate (20 mL×2). The combined organic layers were washed with brine (20 mL 2), dried over anhydrous sodium sulfate, filtered and concentrated under reduced pressure and the residue was purified by prep-TLC (silica gel plate, Petroleum ether/Ethyl acetate 10:1, R_{f}=0.4) to give 6-(cyclopropylmethyl)-3-fluoro-2-iodo-benzonitrile (295 mg, 57.22% yield) as a white solid. ¹H NMR (400 MHz, CDCl₃): δ=7.44 (dd, J=8.4, 4.8 Hz, 1H), 7.25-7.20 (m, 1H), 2.81 (d, J=7.2 Hz, 2H), 1.07-1.03 (m, 1H), 0.62-0.57 (m, 2H), 0.32-0.27 (m, 2H).

Step 5: A mixture of 6-(cyclopropylmethyl)-3-fluoro-2-iodo-benzonitrile (295 mg, 980 μmol, 1.00 eq.), 1-methyl-5-(4,4,5,5-tetramethyl-1,3,2-dioxaborolan-2-yl)-1H-pyrazole (245 mg, 1.18 mmol, 1.20 eq.), potassium carbonate (271 mg, 1.96 mmol, 2.00 eq.) and [1,1-bis(diphenylphosphino)ferrocene]dichloropalladium(II) (71.7 mg, 98.0 μmol, 0.10 eq.) in 1,4-dioxane (5.0 mL) and water (0.50 mL) was degassed and stirred at 80° C. for 2 hours under nitrogen atmosphere. The reaction mixture was quenched by addition of water (10.0 mL) at 25° C., and then extracted with ethyl acetate (20 mL×3). The combined organic layers were washed with brine (20 mL×2), dried over anhydrous sodium sulfate filtered and concentrated under reduced pressure and the residue was purified by prep-TLC (silica gel plate, Petroleum ether/Ethyl acetate 4:1, R_{f}=0.3) to give compound 6-(cyclopropylmethyl)-3-fluoro-2-(1-methyl-1H-pyrazol-5-yl)benzonitrile (100 mg, 38.8% yield) as a yellow solid. ¹H NMR (400 MHz, CDCl₃): δ=7.65 (d, J=2.0 Hz, 1H), 7.60 (dd, J=8.4, 5.2 Hz, 1H), 7.41 (t, J=8.8 Hz, 1H), 6.54 (d, J=1.6 Hz, 1H), 3.83 (s, 3H), 2.85 (d, J=6.8 Hz, 2H), 1.15-1.09 (m, 1H), 0.68-0.63 (m, 2H), 0.37-0.34 (m, 2H).

Step 6: A mixture of 6-(cyclopropylmethyl)-3-fluoro-2-(1-methyl-1H-pyrazol-5-yl)benzonitrile (80.0 mg, 313 μmol, 1.00 eq.), N-Iodosuccinimide (176 mg, 783 μmol, 2.50 eq.) and 4-methylbenzenesulfonic acid hydrate (5.96 mg, 31.3 μmol, 0.10 eq.) in N,N-dimethylformamide (3.0 mL) and acetonitrile (2.0 mL) was degassed and stirred at 90° C. for 12 hours under nitrogen atmosphere. The reaction mixture was quenched by addition saturated sodium thiosulfate solution (20 mL) and then extracted with ethyl acetate (20 mL×3). The combined organic layers were washed with brine (20 mL×2), dried over anhydrous sodium sulfate, filtered and concentrated under reduced pressure and the residue was purified by prep-TLC (silica gel plate, Petroleum ether/Ethyl acetate 3:1, Rf=0.4) to give 6-(cyclopropylmethyl)-3-fluoro-2-(4-iodo-1-methyl-1H-pyrazol-5-yl)benzonitrile (100 mg, 262 μmol, 83.7% yield) as a yellow solid. LCMS [M+1]⁺: 382.3

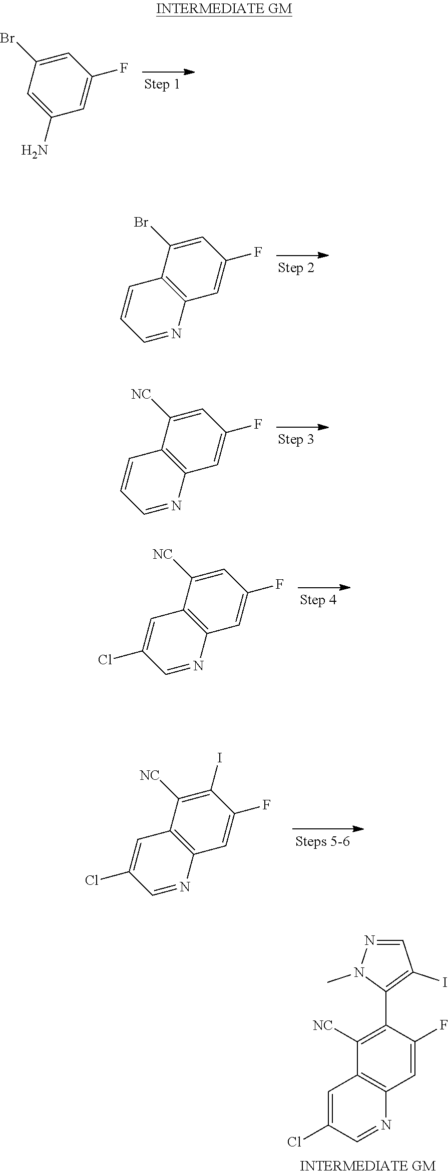

Step 1: To a solution of 3-bromo-5-fluoro-aniline (40.0 g, 210 mmol, 1.00 eq.) in sulfuric acid (413 g, 4.21 mol, 224 mL, 20.0 eq.) was added glycerol (77.6 g, 842 mmol, 63.1 mL, 4.00 eq.), nitrobenzene (51.8 g, 421 mmol, 43.2 mL, 2.00 eq.), followed by ferrous sulfate (3.84 g, 25.3 mmol, 0.12 eq.) carefully at 25° C. The reaction mixture was highly exothermic and the temperature rose rapidly. The reaction mixture was allowed to naturally cooled down for 5 minutes and then the reaction was stirred at 80° C. for 16 hours. The mixture was poured into ice/water (300 ml) slowly. The resulting mixture was basified with sodium hydroxide (12 N) solution at 0° C. until pH=9 and filtered to give a filtrate. The filtrate was extracted with ethyl acetate (200 mL×3). The combined organic layers were washed with brine (50 mL×2). The organic phase was dried over anhydrous sodium sulfate, filtered and concentrated in vacuo. The residue was purified by reverse phase chromatography (Column: I.D. 100 mm×H400 mm Welch ultimate XB_C18 20-40 μm; 120 A; Flow rate: 200 ml/min; Mobile phase: acetonitrile/water (0.225% formic acid), Gradient B %: 5-70%). The collected fractions were concentrated in vacuo to remove acetonitrile. The resulting mixture was basified with saturated sodium bicarbonate solution to pH=8. The resulting mixture was extracted with dichloromethane (150 mL×3). The combined organic layers were dried over anhydrous sodium sulfate, filtered and concentrated in vacuo to afford 5-bromo-7-fluoro-quinoline as a 3:1 mixture with 7-bromo-5-fluoroquinoline (25.0 g, 52% yield) as a brown solid. ¹H NMR (400 MHz, CDCl₃) δ=9.02-8.85 (m, 1H), 8.51 (br d, J=8.4 Hz, 1H), 7.83-7.59 (m, 1H), 7.57-7.43 (m, 1H), 7.38 (dd, J=1.2, 9.2 Hz, 1H); LCMS [ESI, M+1]⁺: 225.9/227.9

Step 2: To a solution of 5-bromo-7-fluoro-quinoline (25.0 g, 111 mmol, 1.00 eq.) in dimethyl formamide (500 mL) was added Zn(CN)₂ (26.0 g, 221 mmol, 14.0 mL, 2.00 eq.), tris(dibenzylideneacetone)dipalladium(0) (10.1 g, 11.0 mmol, 0.10 eq.), 1,1′-bis(diphenylphosphino)ferocene (12.3 g, 22.1 mmol, 0.20 eq.) and zinc powder (723 mg, 11.1 mmol, 0.10 eq.) at 25° C. under nitrogen atmosphere. The mixture was stirred at 100° C. for 2 hours. The reaction mixture was filtered, and the filter cake was washed with ethyl acetate (50 mL×3). Then the filtrate was poured into water (3.00 L). The mixture was extracted with ethyl acetate (700 mL×2). The organic phase was washed with water (100 mL×2), dried over anhydrous sodium sulfate, concentrated in vacuum and the residue was purified by two times flash silica gel chromatography (23% Ethyl acetate in Petroleum ether). The collected mixture was concentrated in vacuo to afford 7-fluoroquinoline-5-carbonitrile as a 3:1 mixture with 5-fluoroquinoline-7-carbonitrile, 15.0 g, 78% yield) as a yellow solid. LCMS [ESI, M+1]⁺: 173.1

Step 3: To a solution of 7-fluoroquinoline-5-carbonitrile (6.00 g, 34.9 mmol, 1.00 eq.) in dimethyl formamide (150 mL) was added N-chlorosuccinimide (14.0 g, 105 mmol, 3.00 eq.) at 25° C. The mixture was stirred at 130° C. for 32 hours. The resulting mixture was poured into water (1.20 L) and filtered to give a filtrate. The filtrate was extracted with ethyl acetate (500 mL×3). The combined organic layers were washed with water (300 mL×2). The organic layer was dried over anhydrous sodium sulfate, filtered and concentrated in vacuo. The residue was purified by two times flash silica gel chromatography (Ethyl acetate/Petroleum ether 17-45%). The collected fractions were concentrated in vacuo to give 12.0 g of a crude residue. The residue was purified by reverse-phase (formic acid condition; Column: I.D. 95 mm×H365 mm Welch ultimate XB_C18 20-40 μm; 120 A; Flow rate: 200 ml/min; Mobile phase: acetronitrile/water (0.225% formic acid); Gradient B %: 10-70% 50 min; 70% 30 min). The collected fractions were concentrated in vacuo to remove acetronitrile. The resulting mixture was extracted with dichloromethane (300 mL×2). The combined organic layers were dried over anhydrous sodium sulfate, filtered and concentrated in vacuo to afford 3-chloro-7-fluoro-quinoline-5-carbonitrile as a 1:1 mixture with 3-chloro-5-fluoroquinoline-7-carbonitrile, 6.60 g, 28.6 mmol, 32% yield, 89% purity) as a yellow solid. ¹H NMR (400 MHz, DMSO-d₆) δ=9.11 (dd, J=2.4, 7.6 Hz, 1H), 8.71 (dd, J=0.8, 2.4 Hz, 1H), 8.50-8.45 (m, 1H), 8.02 (dd, J=1.2, 9.6 Hz, 1H); LCMS [ESI, M+1]⁺: 207.0.

Step 4: To a solution of lithium diisopropylamide (2.00 M, 9.83 mL, 1.40 eq.) in THF (45 mL) was added 3-chloro-7-fluoro-quinoline-5-carbonitrile (2.90 g, 14.0 mmol, 1.00 eq.) in THF (20 mL) at −65° C. under nitrogen atmosphere. The mixture was stirred at −65° C. for 0.5 hour before iodine (5.34 g, 21.1 mmol, 4.24 mL, 1.50 eq.) in THF (10.0 mL) was added to the mixture at −65° C. over a course of 1 hour with stirring under nitrogen atmosphere. The mixture was stirred for 0.5 hour at −65° C. and then slowly warmed up to 20° C. and stirred at 20° C. for 14 hours. The mixture was slowly poured into water (150 mL) and extracted with ethyl acetate (100 mL×3). The combined organic layers were washed with water (20 mL×2), dried over anhydrous sodium sulfate, filtered and concentrated in vacuo. The residue was purified by flash silica gel chromatography (Ethyl acetate/Petroleum ether 27-37%) to afford 3-chloro-7-fluoro-6-iodo-quinoline-5-carbonitrile a 1:1 mixture with 3-chloro-5-fluoro-6-iodoquinoline-7-carbonitrile (2.40 g, crude) as a brown solid; LCMS [ESI, M+1]⁺: 332.8

Step 5: To a solution of 3-chloro-7-fluoro-6-iodo-quinoline-5-carbonitrile (2.40 g, 7.22 mmol, 1.00 eq.) and 1-methyl-5-(4,4,5,5-tetramethyl-1,3,2-dioxaborolan-2-yl)pyrazole (3.00 g, 14.4 mmol, 2.00 eq.) in dioxane (25 mL) and water (5.0 mL) was added saturated sodium bicarbonate (2.43 g, 28.9 mmol, 1.12 mL, 4.00 eq.), followed by Pd(dtbpf)Cl₂ (941 mg, 1.44 mmol, 0.20 eq.) at 20° C. under nitrogen atmosphere. The mixture was stirred at 80° C. for 18 hours. The resulting mixture was poured into water (300 mL) and extracted with ethyl acetate (60 mL×3). The combined organic layers were washed with water (50 mL×3). The organic layer was separated, dried over anhydrous sodium sulfate, filtered and concentrated in vacuo. The residue was purified by flash silica gel chromatography chromatography (Ethyl acetate/Petroleum ether 42%) The collected mixture was concentrated in vacuo to give a residue which was further purified by Prep-HPLC (column: Welch ultimate XB-CN 250×70×10 μm; mobile phase: [hexane-ethanol (0.1% ammonium hydroxide)]; B %: 1%/6-40%, 15 min). The collected fractions were concentrated in vacuo to afford 3-chloro-7-fluoro-6-(2-methylpyrazol-3-yl)quinoline-5-carbonitrile (490 mg, 12% yield) as a brown solid. LCMS [ESI, M+1]⁺: 287.0. ¹H NMR (400 MHz, DMSO-d₆) δ=9.19 (d, J=2.4 Hz, 1H), 8.64-8.53 (m, 1H), 8.49 (d, J=10.4 Hz, 1H), 7.70 (d, J=2.0 Hz, 1H), 6.73 (d, J=1.6 Hz, 1H), 3.79 (d, J=0.8 Hz, 3H) and 3-chloro-5-fluoro-6-(2-methylpyrazol-3-yl)quinoline-7-carbonitrile (620 mg, 15% yield) as a yellow solid. LCMS [ESI, M+1]⁺: 287.0. ¹H NMR (400 MHz, DMSO-d₆) δ=9.21 (d, J=2.2 Hz, 1H), 8.83 (d, J=1.7 Hz, 1H), 8.75 (s, 1H), 7.68 (d, J=1.8 Hz, 1H), 6.66 (d, J=2.0 Hz, 1H).

Step 6: To a solution of 3-chloro-7-fluoro-6-(2-methylpyrazol-3-yl)quinoline-5-carbonitrile (110 mg, 384 μmol, 1.00 eq.) in dimethyl formamide (1.5 mL) was added N-iodosuccinimide (863 mg, 3.84 mmol, 10.0 eq.) at 20° C., followed by acetic acid (38.4 μmol, 2.2 μL, 0.10 eq.) dropwise. The mixture was stirred at 80° C. for 16 hours under nitrogen atmosphere. The mixture was poured into water (10 mL) and extracted with ethyl acetate (10 mL×3). The combined organic layers were washed with water (10 mL×2). The organic layer was dried over anhydrous sodium sulfate, filtered and concentrated in vacuo. The residue was purified by flash silica gel chromatography (Ethyl acetate/Petroleum ether 42%) to afford 3-chloro-7-fluoro-6-(4-iodo-2-methyl-pyrazol-3-yl)quinoline-5-carbonitrile (135 mg, 83% yield,) as a brown solid. LCMS [ESI, M+1]⁺: 412.8.

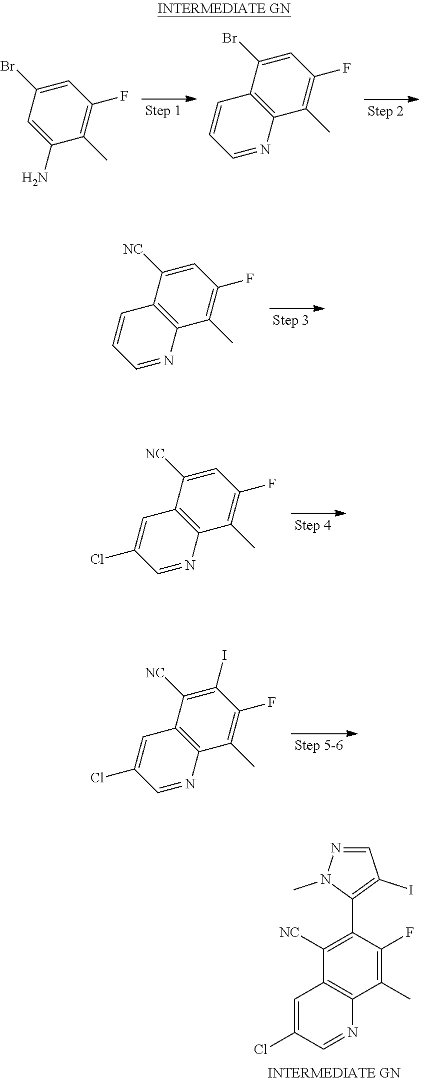

Intermediate GN was prepared following the same procedures for Steps 1-6 as Intermediate GM starting from 5-bromo-3-fluoro-2-methyl-aniline (7.00 g, 34.3 mmol) to give 3-chloro-7-fluoro-6-(4-iodo-2-methyl-pyrazol-3-yl)-8-methyl-quinoline-5-carbonitrile (50 mg, 117 μmol) as a yellow solid. LCMS [M+1]⁺: 427.0

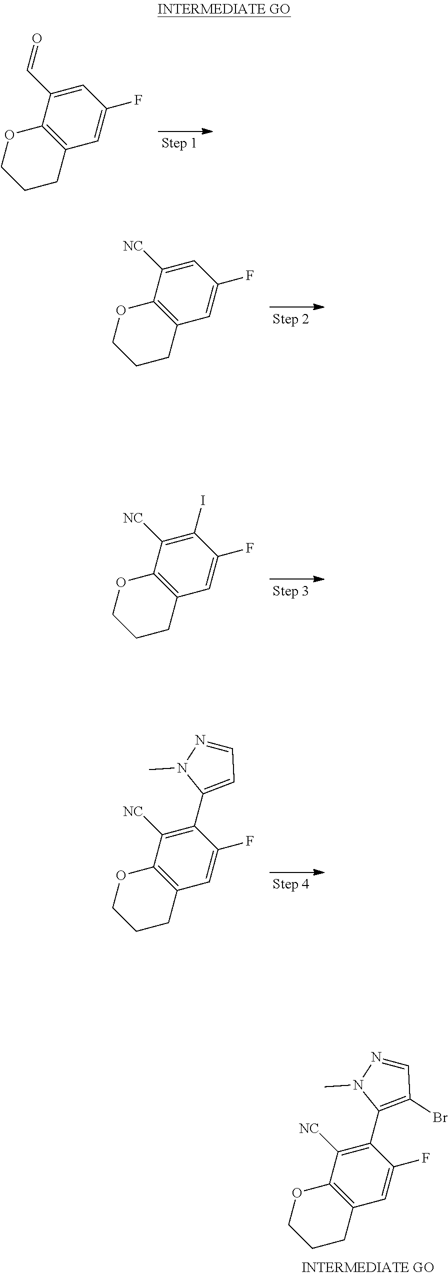

Step 1: To a solution of 6-fluorochromane-8-carbaldehyde (1.50 g, 8.33 mmol, 1.00 eq.) in THF (30 mL) was added ammonium hydroxide (146 g, 1.17 mol, 161 mL, 28% purity, 140 eq.) and iodine (2.54 g, 9.99 mmol, 2.01 mL, 1.20 eq.) at 25° C., the mixture was stirred at 25° C. for 1 hour. The mixture was quenched with saturated aq sodium sulfite. (10.0 mL). The mixture was extracted with ethyl acetate (50 mL×3), the combined organic extracts were washed brine (20 mL), dried over anhydrous sodium sulfate, filtered and concentrated under vacuum to give 6-fluorochromane-8-carbonitrile (1.54 g, crude) as a brown solid. The crude product was used directly without further purification. ¹H NMR (400 MHz, MeOD) δ=7.21-7.15 (m, 2H), 4.31 (t, J=4.2 Hz, 1H)), 2.84 (t, J=6.4 Hz, 1H)), 2.06-2.00 (m, 2H).

Step 2: n-Butyllithium (2.50 M, 2.37 mL, 1.50 eq.) was added to a solution of diisopropylamine (560 mg, 5.93 mmol, 838 μL, 1.50 eq.) in THF (10.0 mL) at −70° C. and the reaction mixture was stirred at −70° C. for 30 minutes. Then 6-fluorochromane-8-carbonitrile (700 mg, 3.95 mmol, 1.00 eq.) in THF (5.0 mL) was added to the mixture. The reaction mixture was stirred for 2 hours at −70° C. A solution of iodine (1.20 g, 4.74 mmol, 955 μL, 1.20 eq.) in THF (5.0 mL) was added to the reaction mixture at −70° C. and the solution was stirred for 30 minutes at −70° C. and for 2 hours at 25° C. The reaction was quenched with saturated ammonium chloride (20 mL). The mixture was extracted with ethyl acetate (3×30 mL). The combined organic extracts were washed with saturated ammonium chloride (20 mL) and brine (20 mL) and then dried over anhydrous sodium sulfate. The residue was purified by flash silica gel chromatography (Ethyl acetate/Petroleum ether 0-20%) to give 6-fluoro-7-iodo-chromane-8-carbonitrile (470 mg, 39% yield) as a yellow oil.

Step 3: A mixture of 6-fluoro-7-iodo-chromane-8-carbonitrile (470 mg, 1.55 mmol, 1.00 eq.), 1-methyl-5-(4,4,5,5-tetramethyl-1,3,2-dioxaborolan-2-yl)pyrazole (1.45 g, 6.98 mmol, 4.50 eq.), sodium bicarbonate (260 mg, 3.09 mmol, 120 μL, 2.00 eq.), (Ad₂n-BuP)—Pd G3 (113 mg, 155 μmol, 0.1 eq.) in dioxane (10.0 mL) and water (1.0 mL) was degassed stirred at 80° C. for 3 hours under nitrogen atmosphere. After being cooled to room temperature, the mixture was concentrated under vacuum. The residue was purified by flash silica gel chromatography (Ethyl acetate/Petroleum ether 0-40%) to give 6-fluoro-7-(2-methylpyrazol-3-yl)chromane-8-carbonitrile (240 mg, 60% yield) as a yellow oil. ¹H NMR (400 MHz, CDCl₃) 5 ppm 7.52 (d, J=1.6 Hz, 1H), 7.04 (d, J=9.2 Hz, 1H), 6.40 (d, J=2.0 Hz, 1H) 4.30 (t, J=4.2 Hz, 2H), 3.72 (s, 3H), 2.80 (t, J=6.4 Hz, 2H), 2.05-1.99 (m, 2H).

Step 4: To a solution of 6-fluoro-7-(2-methylpyrazol-3-yl)chromane-8-carbonitrile (240 mg, 933 μmol, 1.00 eq.) in acetonitrile (5.0 mL) was added NBS (166 mg, 933 μmol, 1.00 eq.). The mixture was stirred at 25° C. for 2 hours. After being cooled to room temperature, the mixture was concentrated under vacuum. The residue was purified by flash silica gel chromatography (Ethyl acetate/Petroleum ether 0-60%) to give 7-(4-bromo-2-methyl-pyrazol-3-yl)-6-fluoro-chromane-8-carbonitrile (220 mg, 69% yield) as a brown solid. ¹H NMR (400 MHz, CDCl₃-d) δ=7.58 (s, 1H), 7.15 (d, J=9.2 Hz, 1H), 4.38 (dd, J=4.8, 5.6 Hz, 2H), 3.78 (s, 3H), 2.89 (t, J=6.4 Hz, 2H), 2.11 (dd, J=4.0, 6.4 Hz, 2H).

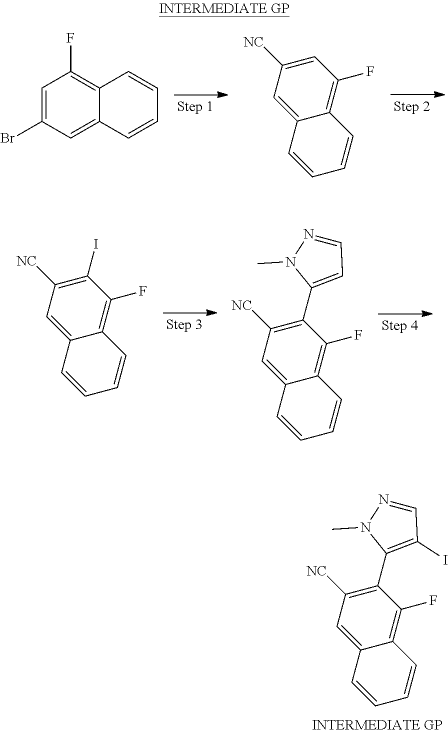 [SMILES: Fc1cc(Br)cc2ccccc12 ; N#Cc1cc(F)c2ccccc2c1 ; N#Cc1cc2ccccc2c(F)c1I ; Cn1nccc1-c1c(C#N)cc2ccccc2c1F ; Cn1ncc(I)c1-c1c(C#N)cc2ccccc2c1F]

Step 1: A mixture of 3-bromo-1-fluoro-naphthalene (1.20 g, 5.33 mmol, 1.00 eq.), Pd(PPh₃)₄ (493 mg, 427 μmol, 0.08 eq.), zinc cyanide (626 mg, 5.33 mmol, 1.00 eq.) in dimethyl formamide (20 mL) was degassed and stirred at 115° C. for 4 hours under nitrogen atmosphere. The mixture was cooled to 25° C., diluted with ethyl acetate (100 mL). The mixture was washed with brine (100 mL×2), dried over anhydrous sodium sulfate, filtered and concentrated and the residue was purified by column chromatography (SiO₂, Ethyl acetate/Petroleum ether 0-10%) to give 4-fluoronaphthalene-2-carbonitrile (750 mg, 82% yield) as a white solid.

¹H NMR (400 MHz, CDCl₃) δ=8.08 (br d, J=8.4 Hz, 1H), 7.96 (br d, J=0.8 Hz, 1H), 7.90-7.80 (m, 1H), 7.73-7.55 (m, 2H), 7.22 (td, J=2.0, 9.8 Hz, 1H)

Step 2: n-Butyllithium (2.5 M, 607.58 μL, 1.30 eq.) was added to a solution of diisopropylamine (166 mg, 1.64 mmol, 1.40 eq.) in THF (5.0 mL) at −70° C. and the reaction mixture was stirred at −70° C. for 15 minutes before 4-fluoronaphthalene-2-carbonitrile (200 mg, 1.17 mmol, 1.00 eq.) in THF (5.0 mL) was added to the mixture. The reaction mixture was stirred for 2 hours at −40° C. Then a solution of iodine (386 mg, 1.52 mmol, 1.30 eq.) in THF (5.0 mL) was added to the reaction mixture at −70° C. and the solution was stirred for 30 minutes at −70° C. and for 10 hours at 25° C. The reaction was quenched with water (2.0 mL), and diluted with ethyl acetate (300 mL), washed with aq. sodium thiosulfate (300 mL×2) and brine (250 mL). The organic phase was dried over anhydrous sodium sulfate filtered and concentrated under reduced pressure and the residue was purified by prep-TLC (SiO₂, Petroleum ether/Ethyl acetate 10:1) to give 4-fluoro-3-iodo-naphthalene-2-carbonitrile (240 mg, 69% yield) as a yellow solid. ¹H NMR (400 MHz, CDCl₃) δ=8.08 (d, J=8.4 Hz, 1H), 7.98 (s, 1H), 7.87-7.83 (m, 1H), 7.67 (dd, J=1.2, 8.4 Hz, 1H), 7.23 (dd, J=1.2, 9.8 Hz, 1H).

Step 3: A mixture of 4-fluoro-3-iodo-naphthalene-2-carbonitrile (400 mg, 1.35 mmol, 1.00 eq.), 1-methyl-5-(4,4,5,5-tetramethyl-1,3,2-dioxaborolan-2-yl)pyrazole (364 mg, 1.75 mmol, 1.3 eq.), (Ad₂n-BuP)—Pd G3 (98.1 mg, 135 μmol, 0.10 eq.) and potassium phosphate (857 mg, 4.04 mmol, 3.00 eq.) in n-butyl alcohol (10.0 mL) was degassed and stirred at 60° C. for 6 hours under nitrogen atmosphere. The reaction mixture was concentrated under reduced pressure and the residue was purified by column chromatography (SiO₂, Ethyl acetate/Petroleum ether 10-30%) to give 4-fluoro-3-(2-methylpyrazol-3-yl)naphthalene-2-carbonitrile (230 mg, 68% yield) as a white solid. LCMS [M+1]⁺=252.0.

Step 4: To a solution of 4-fluoro-3-(2-methylpyrazol-3-yl)naphthalene-2-carbonitrile (210 mg, 836 μmol, 1.00 eq.) in acetonitrile (1.0 mL) was added NIS (1.50 g, 6.69 mmol, 8.00 eq.). The mixture was stirred at 80° C. for 12 hours. The reaction mixture was concentrated under reduced pressure and the residue was purified by column chromatography (SiO₂, Ethyl acetate/Petroleum ether 10-30%) to give 4-fluoro-3-(4-iodo-2-methyl-pyrazol-3-yl)naphthalene-2-carbonitrile (250 mg, 79% yield) as a yellow solid. LCMS [M+1]⁺=377.9. ¹H NMR (400 MHz, CDCl₃) δ=8.29-8.21 (m, 2H), 8.08-8.00 (m, 1H), 7.82 (dt, J=1.2, 7.6 Hz, 2H), 7.71 (s, 1H), 3.87 (s, 3H).

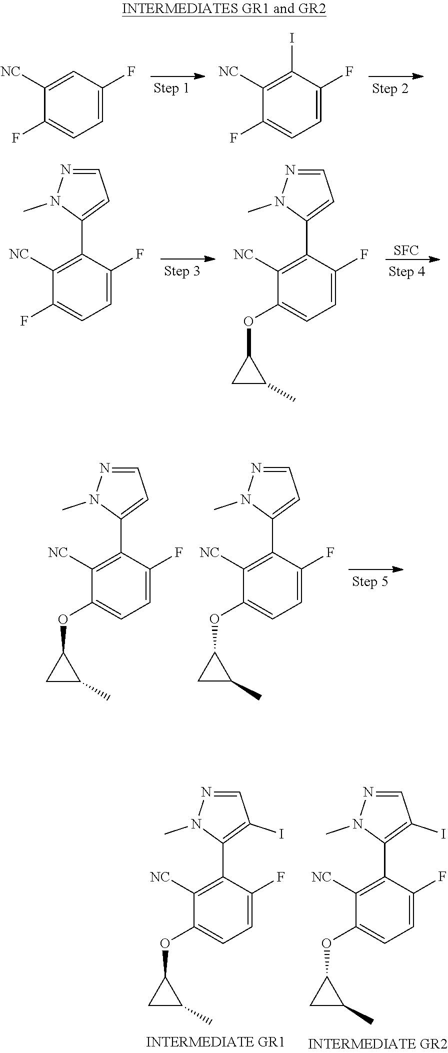

Step 1: To a solution of lithium diisopropylamide (2.00 M, 53.9 mL, 1.50 eq.) in THF (40.0 mL) was added 2,5-difluorobenzonitrile (10.0 g, 71.8 mmol, 1.00 eq.) in THF (40.0 mL) at −65° C. The mixture was stirred at −65° C. for 0.5 h before iodine (36.4 g, 143 mmol, 28.9 mL, 2.00 eq.) in THF (20 mL) was added at −65° C. The mixture was stirred at −65° C. for 1 hour. The mixture was diluted with water (200 mL), extracted with Ethyl acetate (200 mL), organic phase was dried over sodium sulfate, filtered and concentrated. The residue was purified by silica gel chromatography (Ethyl acetate/Petroleum ether 0-50%) to give 3, 6-difluoro-2-iodo-benzonitrile (19.0 g, 50% yield, 50% purity) as a brown oil. LCMS [ESI, M+1]⁺=347.

Step 2: To a solution of 3,6-difluoro-2-iodo-benzonitrile (18.9 g, 71.3 mmol, 1.00 eq.) and 1-methyl-5-(4,4,5,5-tetramethyl-1,3,2-dioxaborolan-2-yl)pyrazole (17.8 g, 85.5 mmol, 1.20 eq.) in dioxane (100 mL) and water (20 mL) was added sodium bicarbonate (15.1 g, 142 mmol, 2.00 eq.) and Pd(dtbpf)Cl₂ (2.32 g, 3.57 mmol, 0.05 eq.) at 25° C. The mixture was stirred at 80° C. for 16 hours. The mixture was diluted with water (200 mL), extracted with ethyl acetate (200 mL), organic phase was dried over sodium sulfate, filtered and concentrated. The residue was purified by silica gel chromatography with (Ethyl acetate/Petroleum ether 0-70%) to give 3,6-difluoro-2-(2-methylpyrazol-3-yl)benzonitrile (8.00 g, 50% yield) as a brown solid. LCMS [ESI, M+1]⁺=220.1. ¹H NMR (400 MHz, DMSO-d₆) δ=7.97-7.84 (m, 1H), 7.77 (dt, J=4.0, 9.0 Hz, 1H), 7.63 (d, J=2.0 Hz, 1H), 6.62 (d, J=2.0 Hz, 1H), 3.74 (d, J=1.2 Hz, 3H).

Step 3: To a solution of trans-2-methylcyclopropanol (300 mg, 4.16 mmol, 1.00 eq.) and 3,6-difluoro-2-(2-methylpyrazol-3-yl)benzonitrile (1.00 g, 4.58 mmol, 1.10 eq.) in dimethyl formamide (9.00 mL) was added cesium carbonate (2.03 g, 6.24 mmol, 1.50 eq). The mixture was stirred at 80° C. for 16 hours. The mixture was diluted with water (100 mL), extracted with ethyl acetate (100 mL), organic phase was dried over sodium sulfate, filtered and concentrated. The residue was purified by prep-HPLC (column: Waters Xbridge C18 150×50 mm×10 μm; mobile phase: [water (10 mM NH₄HCO₃)-ACN]; B %: 35%-65%) to give 3-fluoro-6-[trans-2-methylcyclopropoxy]-2-(2-methylpyrazol-3-yl)benzonitrile (530 mg, 45% yield) as a white solid. LCMS [ESI, M+1]⁺=272.1. ¹HNMR (400 MHz, DMSO-d₆) δ=7.78 (t, J=9.2 Hz, 1H), 7.61-7.51 (m, 2H), 6.53 (d, J=2.0 Hz, 1H), 3.88-3.78 (m, 1H), 3.75-3.66 (m, 3H), 1.21-1.06 (m, 4H), 1.00-0.86 (m, 1H), 0.70 (q, J=6.0 Hz, 1H).

Step 4: 3-fluoro-6-[trans-2-methylcyclopropoxy]-2-(2-methylpyrazol-3-yl)benzonitrile (1.40 g, 5.16 mmol) was purified by SFC (column: DAICEL CHIRALPAK AD-H (250 mm×30 mm, 5 μm); mobile phase: [0.1% NH₃H₂O EtOH]; B %: 15%-15%, 2.3; 240 min) to give 3-fluoro-6-[(1R,2R)-2-methylcyclopropoxy]-2-(2-methylpyrazol-3-yl)benzonitrile (490 mg, 35% yield) as a white solid and 3-fluoro-6-[(1S,2S)-2-methylcyclopropoxy]-2-(2-methylpyrazol-3-yl)benzonitrile (690 mg, 49% yield) as a white solid. LCMS [ESI, M+1]⁺=272.2.

Step 5: To a solution of 3-fluoro-6-[(1R,2R)-2-methylcyclopropoxy]-2-(2-methylpyrazol-3-yl)benzonitrile (490 mg, 1.81 mmol, 1.00 eq.) in dimethyl formamide (10.0 mL) was added NIS (4.06 g, 18.0 mmol, 10.0 eq.) and acetic acid (21.6 mg, 361 μmol, 20.6 μL, 0.20 eq.). The mixture was stirred at 80° C. for 2 h. The mixture was diluted with ethyl acetate (100 mL), washed with sat. sodium thiosulfate (50 mL), organic phase was dried over sodium sulfate, filtered and concentrated. The residue was purified by silica gel chromatography (Ethyl acetate/Petroleum ether 0-50/6) to give 3-fluoro-2-(4-iodo-2-methyl-pyrazol-3-yl)-6-[(1R,2R)-2-methylcyclopropoxy]benzonitrile (640 mg, 71% yield) as a yellow solid. LCMS [ESI, M+1]⁺=397.9. ¹HNMR (400 MHz, CDCl₃) δ=7.65 (s, 1H), 7.47-7.36 (m, 3H), 3.82 (s, 3H), 3.55 (dd, J=2.4, 5.6 Hz, 1H), 1.20-1.16 (m, 4H), 1.12-1.04 (m, 1H), 0.70 (q, J=6.0 Hz, 1H)

The same procedure starting from 3-fluoro-6-[(1S,2S)-2-methylcyclopropoxy]-2-(2-methylpyrazol-3-yl)benzonitrile (350 mg, 1.29 mmol) afforded 3-fluoro-2-(4-iodo-2-methyl-pyrazol-3-yl)-6-[(1S,2S)-2-methylcyclopropoxy]benzonitrile (460 mg, 72% yield) as a yellow solid. LCMS [ESI, M+1]⁺=397.9. ¹H NMR (400 MHz, CDCl₃) δ=7.66-7.63 (m, 1H), 7.48-7.35 (m, 2H), 3.86-3.81 (m, 3H), 3.58-3.51 (m, 1H), 1.22-1.16 (m, 3H), 1.12-1.04 (m, 1H), 0.71 (q, J=6.0 Hz, 1H).

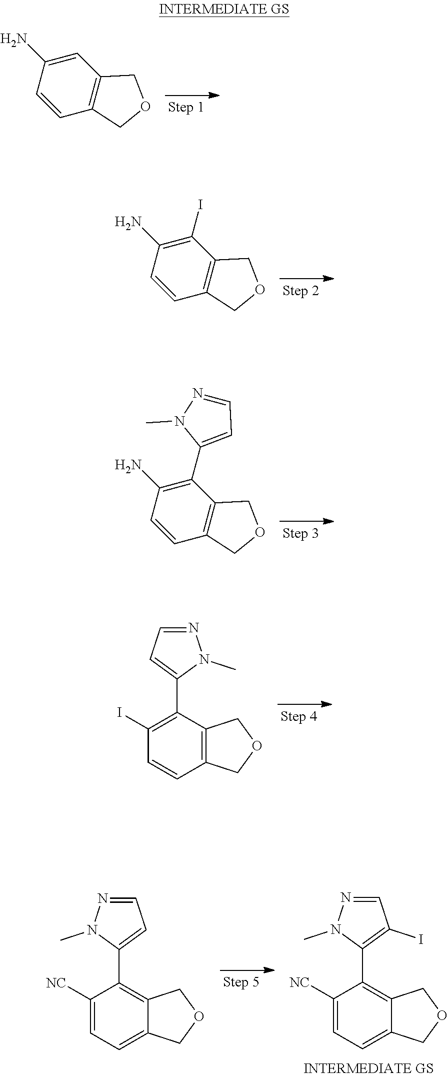 [SMILES: Nc1ccc2c(c1)COC2 ; Nc1ccc2c(c1I)COC2 ; Cn1nccc1-c1c(N)ccc2c1COC2 ; Cn1nccc1-c1c(I)ccc2c1COC2 ; Cn1nccc1-c1c(C#N)ccc2c1COC2 ; Cn1ncc(I)c1-c1c(C#N)ccc2c1COC2]

Step 1: A mixture of 1,3-dihydroisobenzofuran-5-amine (4.50 g, 33.3 mmol, 1.00 eq.) and sodium hydrogen carbonate (4.20 g, 49.9 mmol, 1.94 mL, 1.50 eq.) in dichloromethane (90.0 mL) and methanol (22.5 mL) was degassed and then iodine chloride (5.95 g, 36.6 mmol, 1.87 mL, 1.10 eq.) was added into the above mixture. The mixture was stirred at 15° C. for 4 hours under nitrogen atmosphere. The reaction mixture was diluted with water (50 mL) and extracted with ethyl acetate (50 mL×3). The combined organic layers were washed with brine (50 mL×3), dried over sodium sulfate, filtered and concentrated under reduced pressure and the residue was purified by column chromatography (SiO₂, Petroleum ether/Ethyl acetate 1-50%) to give 4-iodo-1,3-dihydroisobenzofuran-5-amine (3.40 g, 34% yield) as a yellow solid. LCMS [M+1]⁺=261.9. ¹H NMR (400 MHz, CDCl₃) δ=6.88 (d, J=8.0 Hz, 1H), 6.56 (d, J=8.0 Hz, 1H), 5.11 (t, J=1.6 Hz, 2H), 4.88 (s, 2H), 4.39-3.64 (s, 2H).

Step 2: A mixture of 4-iodo-1,3-dihydroisobenzofuran-5-amine (2.00 g, 7.66 mmol, 1.00 eq.), 1-methyl-5-(4,4,5,5-tetramethyl-1,3,2-dioxaborolan-2-yl)pyrazole (1.91 g, 9.19 mmol, 1.20 eq.), potassium phosphate (4.88 g, 23.0 mmol, 3.00 eq.) and (Ad₂ n-BuP)—Pd G3 (558 mg, 766 μmol, 0.10 eq.) in dioxane (30 mL) and water (6.0 mL) was degassed and stirred at 80° C. for 16 hours under nitrogen atmosphere. After cooling down to 15° C., the reaction mixture was diluted with water (50 mL) and extracted with ethyl acetate (50 mL×3). The combined organic layers were washed with brine (50 mL 3), dried over sodium sulfate, filtered and concentrated under reduced pressure and the residue was purified by column chromatography (SiO₂, Methanol/Dichloromethane 1-5%) to give 4-(2-methylpyrazol-3-yl)-1,3-dihydroisobenzofuran-5-amine (1.02 g, 95% yield) as a yellow solid. LCMS [M+1]⁺=216.0.

Step 3: A mixture of 4-(2-methylpyrazol-3-yl)-1,3-dihydroisobenzofuran-5-amine (1.00 g, 4.65 mmol, 1.00 eq.), sodium nitrite (385 mg, 5.57 mmol, 1.20 eq.) and hydrochloric acid solution (687 mg, 6.97 mmol, 673 μL, 37.0% purity, 1.50 eq.) in water (20 mL) was purged with nitrogen for 3 times, The mixture was stirred at 0° C. for 1 hour under nitrogen atmosphere before potassium iodide (3.08 g, 18.6 mmol, 4.00 eq.) was added. The mixture was stirred at 0-15° C. for 4 hours under nitrogen atmosphere. The reaction mixture was quenched by addition disodium sulfite solution (30 mL) at 0° C. The reaction mixture was extracted with ethyl acetate (20 mL×3). The combined organic layers were washed with brine (20 mL×3), dried over sodium sulfate, filtered and concentrated under reduced pressure and the residue was purified by column chromatography (SiO₂, Ethyl acetate/Petroleum 20-60%) to give 5-(5-iodo-1,3-dihydroisobenzofuran-4-yl)-1-methyl-pyrazole (860 mg, 54% yield) as a yellow solid. LCMS [M+1]⁺=326.9.

Step 4: A mixture of 5-(5-iodo-1,3-dihydroisobenzofuran-4-yl)-1-methyl-pyrazole (500 mg, 1.53 mmol, 1.00 eq.), tetrakis(triphenylphosphine)palladium(0) (177 mg, 153 μmol, 0.10 eq.) and zinc cyanide (270. mg, 2.30 mmol, 1.50 eq.) in N,N-dimethylformamide (10.0 mL) was degassed and stirred at 100° C. for 2 hours under nitrogen atmosphere. After cooling down to 15° C., the reaction mixture was filtered and diluted with water (50 mL) and extracted with ethyl acetate (50 mL×3). The combined organic layers were washed with brine (50 mL×3), dried over sodium sulfate, filtered and concentrated under reduced pressure and the residue was purified by column chromatography (SiO₂, Ethyl acetate/Petroleum 20-60%) to give 4-(2-methylpyrazol-3-yl)-1,3-dihydroisobenzofuran-5-carbonitrile (330 mg, 95% yield) as a white solid. LCMS [M+1]⁺: 226.0

Step 5: A mixture of 4-(2-methylpyrazol-3-yl)-1,3-dihydroisobenzofuran-5-carbonitrile (300 mg, 1.33 mmol, 1.00 eq.), N-iodosuccinimide (599 mg, 2.66 mmol, 2.00 eq.), acetic acid (2.10 g, 35.0 mmol, 2.0 mL, 26.3 eq.) in acetonitrile (2.0 mL) was degassed and stirred at 15° C. for 2 hours under nitrogen atmosphere. The reaction mixture was concentrated under reduced pressure to remove acetonitrile. The residue was diluted with water (50 mL) and extracted with ethyl acetate (30 mL×3). The combined organic layers were washed with brine (30 mL 3), dried over sodium sulfate, filtered and concentrated under reduced pressure and the residue was purified by column chromatography (SiO₂, Ethyl acetate/Petroleum 20-60%) to give 4-(4-iodo-2-methyl-pyrazol-3-yl)-1,3-dihydroisobenzofuran-5-carbonitrile (460 mg, 1.30 mmol, 97% yield) as a white solid. LCMS [M+1]⁺: 351.9.

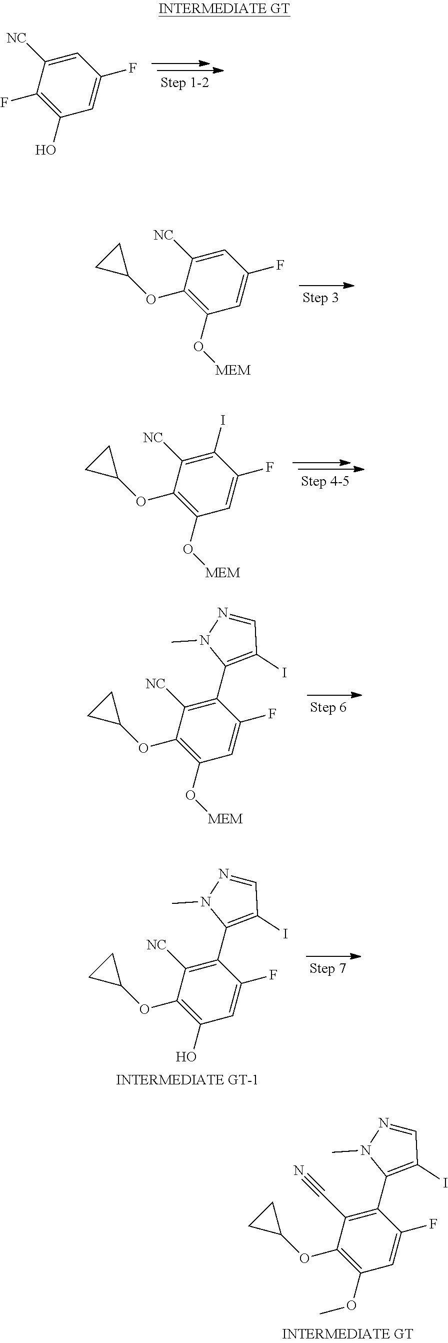

Step 1: To a solution of 2,5-difluoro-3-hydroxy-benzonitrile (32.0 g, 206 mmol, 1 eq.) in N,N-dimethylformamide (100 mL) was added potassium carbonate (71.3 g, 516 mmol, 2.50 eq.) at 20° C. After addition, the mixture was stirred at this temperature for 0.5 hr, and then 1-(chloromethoxy)-2-methoxy-ethane (38.6 g, 309 mmol, 335 mL, 1.5 eq.) was added dropwise at 20° C. The resulting mixture was stirred at 20° C. for 1.5 hr. The reaction mixture was quenched by addition water (100.0 mL) at 20° C., and then diluted with ethyl acetate (800 mL). The solution was washed by brine (100 mL×2), aq. calcium chloride (200.0 mL×3) and brine (100.0 mL×3). The organic layers was dried over Sodium sulfate, filtered and concentrated under reduced pressure to give 2,5-difluoro-3-(2-methoxyethoxymethoxy)benzonitrile (48.0 g, 96% yield) as a light yellow oil. ¹H NMR (400 MHz, DMSO-d) 6:7.58-7.76 (m, 2H), 5.53 (s, 2H), 3.85-3.91 (m, 2H), 3.54-3.60 (m, 2H), 3.31 (s, 3H).

Step 2: To a solution of cyclopropanol (21.5 g, 370 mmol, 2.00 eq.) in N,N-dimethylformamide (200 mL) was added NaH (18.5 g, 463 mmol, 60% purity, 2.50 eq.) at 0° C. over 15 min. After addition, the mixture was stirred at this temperature for 15 min, and then 2,5-difluoro-3-(2-methoxyethoxymethoxy)benzonitrile (45.0 g, 185 mmol, 1.00 eq.) was added at 0° C. The resulting mixture was stirred at 0° C. for 1 hr. The mixture was poured into ice-water (w/w=1/1) (500 mL) and stirred for 20 min. The aqueous phase was extracted with ethyl acetate (300 mL×3). The combined organic phase was washed with aq. calcium chloride (300 mL×2, w/w=10.0%), brine (300 mL×3), dried over anhydrous sodium sulfate, filtered and concentrated in vacuum. The residue was purified by silica gel chromatography (Ethyl acetate/Petroleum ether 5-30%) and prep-HPLC (TFA as additive). The organic solvent was concentrated under reduced pressure and the aqueous phase was extracted with ethyl acetate (200 mL×3). The combined organic phase was washed with brine (100 mL×3), dried with anhydrous sodium sulfate, filtered and concentrated to give 2-(cyclopropoxy)-5-fluoro-3-(2-methoxyethoxymethoxy)benzonitrile (16.0 g, 31% yield) as a colorless oil. ¹H NMR (400 MHz, DMSO-d₆) δ: 7.27-7.56 (m, 2H), 5.42 (s, 2H), 4.37 (tt, J=6.0, 2.8 Hz, 1H), 3.77-3.85 (m, 2H), 3.45-3.57 (m, 2H), 3.22 (s, 3H), 0.76-0.88 (m, 2H), 0.61 (q, J=6.00 Hz, 2H).

Step 3: A solution of lithium 2,2,6,6-tetramethylpiperidin-1-ide (1.10 M, 44.4 mL, 1.1 eq.) in THF (100 mL) at −78° C. was added a solution of 2-(cyclopropoxy)-5-fluoro-3-(2-methoxyethoxymethoxy)benzonitrile (12.5 g, 44.4 mmol, 1.00 eq.) in THF (100 mL) at −78° C. over 15 min. The dark yellow solution was stirred at −78° C. for 30 min before a solution of iodine (11.8 g, 46.7 mmol, 9.40 mL, 1.05 eq.) in THF (100 mL) was added dropwise over the course of 15 min. The mixture was stirred for 30 min at −78° C. before being allowed to warm up to 20° C. Stirring was continued for 1 h. The reaction was quenched with aqueous aq. hydrochloric acid (0.2 N). The mixture was diluted with water (100.0 mL) and extracted by ethyl acetate (200.0 mL×3). The combined organic layer was washed again with aqueous sodium sulfite, brine (100.0 mL 3) and dried with sodium sulfate, filtered and evaporated. The residue was purified by silica gel chromatography (Ethyl acetate/Petroleum ether 10-33%) to give 2-(cyclopropoxy)-5-fluoro-6-iodo-3-(2-methoxyethoxymethoxy)benzonitrile (5.60 g, 31% yield) as a yellow oil. ¹H NMR (400 MHz, DMSO-d₆) δ: 7.53 (d, J=9.6 Hz, 1H), 5.42 (s, 2H), 4.40 (tt, J=6.0, 2.8 Hz, 1H), 3.75-3.80 (m, 2H), 3.46-3.49 (m, 2H), 3.22 (s, 3H), 0.79-0.83 (m, 2H), 0.59-0.65 (m, 2H).

Step 4: To a mixture of 2-(cyclopropoxy)-5-fluoro-6-iodo-3-(2-methoxyethoxymethoxy)benzonitrile (5.60 g, 13.8 mmol, 1 eq.) and 1-methyl-5-(4,4,5,5-tetramethyl-1,3,2-dioxaborolan-2-yl)pyrazole (8.58 g, 41.3 mmol, 3.0 eq.) in dioxane (40.0 mL) and water (4.00 mL) was added potassium phosphate (8.76 g, 41.3 mmol, 3.00 eq.) and (Ad₂n-BuP)—Pd G3 (460 mg, 688 μmol, 0.05 eq.) in one portion at 20° C. under N₂. The mixture was heated to 80° C. and stirred for 3 hours. The mixture was cooled to 20° C., diluted with ethyl acetate (200 mL) and washed with brine (100 mL×3), dried with anhydrous sodium sulfate, filtered and concentrated in vacuum. The residue was purified by silica gel chromatography (Ethyl acetate/Petroleum ether 10-50%) to give 2-(cyclopropoxy)-5-fluoro-3-(2-methoxyethoxymethoxy)-6-(2-methylpyrazol-3-yl) benzonitrile (4.50 g, 91% yield) as a light yellow oil. LCMS [M+1]⁺: 362.3.

Step 5: To a mixture of 2-(cyclopropoxy)-5-fluoro-3-(2-methoxyethoxymethoxy)-6-(2-methylpyrazol-3-yl) benzonitrile (4.40 g, 12.2 mmol, 1 eq.) and NIS (4.11 g, 18.3 mmol, 1.50 eq) in N,N-dimethylformamide (20 mL) was added p-toluenesulfonic acid monohydrate (232 mg, 1.22 mmol, 0.10 eq.) in one portion at 20° C. under N₂. The mixture was heated to 45° C. and stirred for 4 hours. The mixture was diluted with ethyl acetate (250 mL), washed with aq. sodium sulfite (80 mL×4, 10.0% wt) and brine (100 mL×3), dried with anhydrous sodium sulfate, filtered and concentrated in vacuum to give 2-(cyclopropoxy)-5-fluoro-6-(4-iodo-2-methyl-pyrazol-3-yl)-3-(2-methoxyethoxymethoxy)benzonitrile (5.80 g, 98% yield) as a yellow oil. LCMS [M+1]⁺: 488.1. ¹H NMR (400 MHz, DMSO-d₆) δ: 7.72 (s, 1H), 7.64 (d, J=11.2 Hz, 1H), 5.49-5.54 (m, 2H), 4.47 (tt, J=6.0, 2.8 Hz, 1H), 3.82 (dd, J=5.2, 3.6 Hz, 2H), 3.74 (s, 3H), 3.48-3.51 (m, 2H), 3.22 (s, 3H), 0.81-0.89 (m, 2H), 0.60-0.70 (m, 2H).

Step 6: To the 2-(cyclopropoxy)-5-fluoro-6-(4-iodo-2-methyl-pyrazol-3-yl)-3-(2-methoxyethoxymethoxy)benzonitrile (5.50 g, 11.3 mmol, 1.00 eq.) was added TFA (123 g, 1.08 mol, 80 mL, 95.7 eq.) in one portion at 20° C. under N₂. The mixture was stirred at 20° C. for 2.5 hrs. The mixture was concentrated under reduced pressure to give a crude product that was purified by re-crystallization from petroleum ether/tert-butyl methyl ether (5:1, 80 mL) at 15° C. to give 2-(cyclopropoxy)-5-fluoro-3-hydroxy-6-(4-iodo-2-methyl-pyrazol-3-yl)benzonitrile (4.10 g, 91% yield) as a yellow solid. LCMS [M+1]⁺: 400.1. ¹H NMR (400 MHz, DMSO-d₆) δ: 11.39-11.57 (m, 1H), 7.62-7.75 (m, 1H), 7.16-7.25 (m, 1H), 4.39-4.52 (m, 1H), 3.67-3.81 (m, 3H), 0.75-0.88 (m, 2H), 0.54-0.70 (m, 2H).

Step 7: To a mixture of 2-(cyclopropoxy)-5-fluoro-3-hydroxy-6-(4-iodo-2-methyl-pyrazol-3-yl)benzonitrile (0.20 g, 501 μmol, 1.00 eq.) and iodomethane (142 mg, 1.00 mmol, 62.4 μL, 2.00 eq.) in dimethylformamide (5.0 mL) was added potassium carbonate (208 mg, 1.50 mmol, 3.00 eq.) in one portion at 20° C. under nitrogen. The mixture was heated to 20° C. and stirred for 12 hours. The mixture was diluted with ethyl acetate (200 mL) and washed with water (50 mL×2), aq. calcium chloride (50 mL×2) and brine (50 mL×2), dried with anhydrous sodium sulfate, filtered and concentrated in vacuum to give crude 2-(cyclopropoxy)-5-fluoro-6-(4-iodo-2-methyl-pyrazol-3-yl)-3-methoxy-benzonitrile (0.20 g, 97% yield) as a light yellow oil which was used into the next step without further purification. LCMS [M+1]⁺: 414.2.

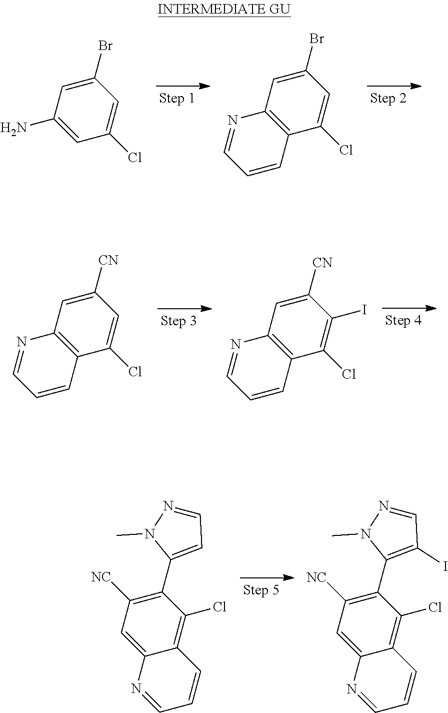 [SMILES: Nc1cc(Cl)cc(Br)c1 ; Clc1cc(Br)cc2ncccc12 ; N#Cc1cc(Cl)c2cccnc2c1 ; N#Cc1cc2ncccc2c(Cl)c1I ; Cn1nccc1-c1c(C#N)cc2ncccc2c1Cl ; Cn1ncc(I)c1-c1c(C#N)cc2ncccc2c1Cl]

Step 1: 3-bromo-5-chloro-aniline (5.00 g, 24.2 mmol, 1.00 eq.) was dissolved in sulfuric acid (50 mL, 70.0% purity). Following that glycerol (5.58 g, 60.5 mmol, 4.53 mL, 2.50 eq.) and nitrobenzene (2.98 g, 24.2 mmol, 2.48 mL, 1.00 eq.) were added. The reaction mixture was degassed and stirred at 130° C. for 4 hours. The reaction mixture was cooled to 15° C. and then dissolved in ethyl acetate (200 mL). The pH of the mixture was adjusted to 6-7 with 30% sodium hydroxide. The solid was filtered off over celite and the organic layer was separated and concentrated to get a crude product. The crude product was purified by silica gel column chromatography (Ethyl acetate/Petroleum ether 5-30%) to get 7-bromo-5-chloro-quinoline and 5-bromo-7-chloroquinoline (5.22 g, 10.8 mmol, 89% yield) as a 1:1 mixture with 7-bromo-5-chloro-quinoline as a white solid. ¹H NMR (400 MHz, CDCl₃) δ=8.97-8.94 (m, 1H), 8.62-8.42 (m, 1H), 8.31-8.04 (m, 1H), 7.89-7.70 (m, 1H), 7.59-7.45 (m, 1H).

Step 2: To a solution of 5-bromo-7-chloro-quinoline (2.80 g, 11.6 mmol, 0.50 eq.) in dimethylformamide (60 mL) was added zinc cyanide (1.63 g, 13.9 mmol, 879 μL, 0.60 eq.) and tetrakis[triphenylphosphine]palladium(0) (2.67 g, 2.31 mmol, 0.10 eq.) and the mixture was stirred at 100° C. for 3 hours under nitrogen. The reaction mixture was cooled to 15° C. and dissolved in ethyl acetate (200 mL). The mixture was washed with brine (100 mL×2). The organic layer was separated and evaporated. The residue was purified by flash silica gel chromatography (Ethyl acetate/Petroleum ether 0-50%). The residue was purified by prep-NPLC (column: Welch Ultimate XB-CN 250×50×10 μm; mobile phase: [Hexane-EtOH]; B %: 1%-10%, 13 min) to give 5-chloroquinoline-7-carbonitrile (600 mg, 3.18 mmol, 14% yield, ¹H NMR (400 MHz, CDCl₃) δ=9.09 (dd, J=1.6, 4.4 Hz, 1H), 8.63 (d, J=8.8 Hz, 1H), 8.42 (s, 1H), 7.79 (d, J=1.6 Hz, 1H), 7.67 (dd, J=4.8, 8.8 Hz, 1H) and 7-chloroquinoline-5-carbonitrile (600 mg, 3.18 mmol, 14% yield, ¹H NMR (400 MHz, CDCl₃) δ=9.07 (dd, J=1.6, 4.4 Hz, 1H), 8.54 (d, J=8.4 Hz, 1H), 8.37 (d, J=1.6 Hz, 1H), 7.96 (d, J=2.0 Hz, 1H), 7.64 (dd, J=4.4, 8.4 Hz, 1H) as white solids. LCMS [M+1]⁺: 189.1.

Step 3: n-Butyllithium (2.50 M, 700 μL, 1.50 eq.) was added to a solution of diisopropylamine (177 mg, 1.75 mmol, 247 μL, 1.50 eq.) in THF (6.0 mL) at −70° C. and the reaction mixture was stirred at −70° C. for 30 minutes. Then 5-chloroquinoline-7-carbonitrile (220 mg, 1.17 mmol, 1.00 eq.) in THF (4.00 mL) was added to the mixture. And the reaction mixture was stirred for 1 hour at −70° C. Then a solution of iodine (592 mg, 2.33 mmol, 470 μL, 2.00 eq.) in THF (2.0 mL) was added to the reaction mixture at −70° C. and the reaction mixture was stirred for 30 minutes at −70° C. and for 2 hours at 25° C. The reaction was quenched with saturated ammonium chloride (5.0 mL). The mixture was extracted with ethyl acetate (3×20 mL). The combined organic extracts were washed with saturated ammonium chloride (6.0 mL) and brine (20 mL) and then dried over sodium sulfate, filtered and concentrated in vacuum and the crude product 5-chloro-6-iodo-quinoline-7-carbonitrile (200 mg, crude), a brown oil, was used directly without further purification. LCMS [M+1]⁺: 314.9. ¹H NMR (400 MHz, CDCl₃) δ=9.06 (dd, J=1.6, 4.0 Hz, 1H), 8.64 (d, J=8.8 Hz, 1H), 8.37 (s, 1H), 7.64 (dd, J=4.0, 8.8 Hz, 1H).

Step 4: To a solution of 5-chloro-6-iodo-quinoline-7-carbonitrile (300 mg, 954 μmol, 1.00 eq.) and 1-methyl-5-(4,4,5,5-tetramethyl-1,3,2-dioxaborolan-2-yl)pyrazole (298 mg, 1.43 mmol, 1.50 eq.) in dioxane (6.0 mL) and water (3.0 mL) was added sodium bicarbonate (240 mg, 2.86 mmol, 111 μL, 3.00 eq.) (Ad₂n-BuP)—Pd G3 (69.5 mg, 95.4 μmol, 0.10 eq.) at 20° C. under nitrogen. The mixture was stirred at 65° C. for 4 hours before being diluted with water (40.0 mL) and extracted with ethyl acetate (50 mL×3). The combined organic extracts were washed with brine (20.0×3 mL) and then dried over anhydrous sodium sulfate, filtered and concentrated in vacuum and the crude product 5-chloro-6-(1-methyl-1H-pyrazol-5-yl)quinoline-7-carbonitrile (180 mg, crude), a yellow solid, was used directly without further purification. LCMS [M+1]⁺: 269.1. ¹H NMR (400 MHz, CDCl₃) δ=9.15 (dd, J=1.6, 4.0 Hz, 1H), 8.73 (d, J=8.8 Hz, 1H), 8.56 (s, 1H), 7.74 (dd, J=4.0, 8.8 Hz, 1H), 7.67 (d, J=2.0 Hz, 1H), 6.49 (d, J=2.0 Hz, 1H), 3.77 (s, 3H).

Step 5: To a solution of 5-chloro-6-(1-methyl-1H-pyrazol-5-yl)quinoline-7-carbonitrile (180 mg, 670 μmol, 1.00 eq.) in acetonitrile (6.0 mL) and dimethylformamide (3.0 mL) was added NIS (301 mg, 1.34 mmol, 2.00 eq.) and p-toluenesulfonic acid monohydrate (127 mg, 670 μmol, 1.00 eq.) at 20° C. The mixture was stirred at 80° C. for 4 hours. The mixture was diluted with water (20 mL) and extracted with ethyl acetate (30 mL×3). The combined organic extracts were washed brine (10 mL×3) and then dried over anhydrous sodium sulfate, filtered and concentrated in vacuum to give 5-chloro-6-(4-iodo-2-methyl-pyrazol-3-yl)-7-cyano-quinoline (290 mg, crude) as a yellow solid. LCMS [M+1]⁺: 395.1. ¹H NMR (400 MHz, CDCl₃-d) δ=9.18 (dd, J=1.2, 4.0 Hz, 1H), 8.73 (d, J=8.8 Hz, 1H), 8.59 (s, 1H), 8.01 (s, 1H), 7.76 (dd, J=4.4, 8.8 Hz, 1H), 3.80 (s, 3H).

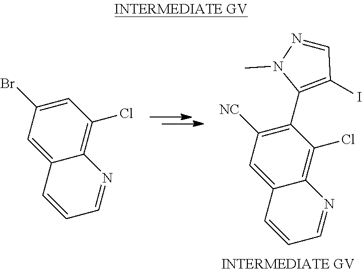 [SMILES: Clc1cc(Br)cc2cccnc12 ; Cn1ncc(I)c1-c1c(C#N)cc2cccnc2c1Cl]

Intermediate GV was prepared following the same procedure as for Steps 2-5 of Intermediate GU starting from 6-bromo-8-chloroquinoline (10.0 g, 41.2 mmol) to give 8-chloro-7-(4-iodo-1-methyl-1H-pyrazol-5-yl)quinoline-6-carbonitrile (130 mg, 329 μmol) as a yellow solid. ¹H NMR (400 MHz, CDCl₃) δ=9.29 (dd, J=4.4, 2.0 Hz, 1H), 8.38 (dd, J=8.4, 1.6 Hz, 1H), 8.35 (s, 1H), 7.75 (dd, J=8.0, 4.0 Hz, 1H), 7.73 (s, 1H), 3.83 (s, 3H).

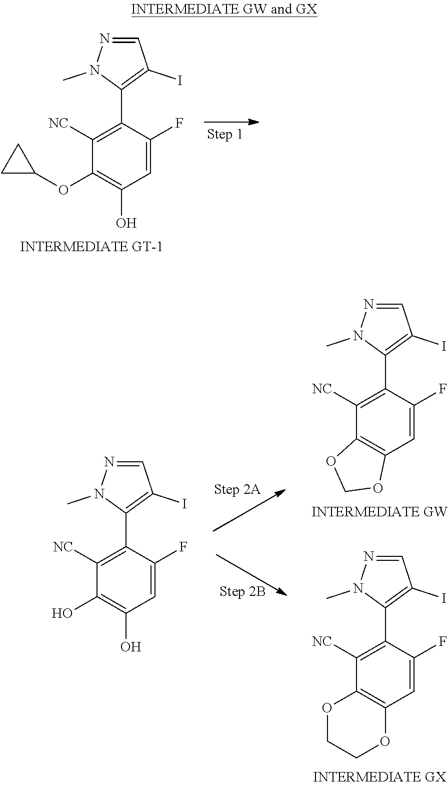 [SMILES: Cn1ncc(I)c1-c1c(F)cc(O)c(OC2CC2)c1C#N ; Cn1ncc(I)c1-c1c(F)cc(O)c(O)c1C#N ; Cn1ncc(I)c1-c1c(F)cc2c(c1C#N)OCCO2 ; Cn1ncc(I)c1-c1c(F)cc2c(c1C#N)OCO2]

Step 1: To a mixture of 2-(cyclopropoxy)-5-fluoro-3-hydroxy-6-(4-iodo-2-methyl-pyrazol-3-yl)benzonitrile (300 mg, 752 μmol, 1.00 eq.) in trifluoromethanesulfonic acid (6.0 mL) was stirred at 50° C. for 3 hours. The reaction mixture was diluted with water (5.0 mL) and adjusted to pH=7 with saturated bicarbonate (10.0 mL). The mixture was then extracted with ethyl acetate (20 mL×3). Combined organic phase was washed with brine (20 mL), dried over anhydrous sodium sulfate, filtered and concentrated to give 5-fluoro-2,3-dihydroxy-6-(4-iodo-2-methyl-pyrazol-3-yl)benzonitrile (300 mg, crude) as a yellow solid which was used in the next step directly without further purification. LC-MS [M+1]⁺=360.0

Step 2A: A mixture of 5-fluoro-2,3-dihydroxy-6-(4-iodo-2-methyl-pyrazol-3-yl)benzonitrile (150 mg, 418 mol, 1.00 eq.), diiodomethane (280 mg, 1.04 mmol, 84.3 μL, 2.50 eq.) and potassium carbonate (144 mg, 1.04 mmol, 2.50 eq.) in dimethyl formamide (3.0 mL) was degassed and stirred at 95° C. for 3 hours under nitrogen atmosphere. The mixture was extracted with ethyl acetate (10.0 mL×3), washed with brine, dried over sodium sulfate, filtered and concentrated under reduced pressure to give 6-fluoro-5-(4-iodo-2-methyl-pyrazol-3-yl)-1,3-benzodioxole-4-carbonitrile, Intermediate GW (105 mg, 37% yield, 54% purity) as a yellow solid. LCMS [M+1]⁺=372.0. ¹H NMR (400 MHz, CDCl₃-d) δ=7.64 (s, 1H), 6.94 (d, J=8.4 Hz, 1H), 6.28 (d, J=2.0 Hz, 2H), 3.83 (s, 3H).

Step 2B: To a solution of 5-fluoro-2,3-dihydroxy-6-(4-iodo-2-methyl-pyrazol-3-yl)benzonitrile (100 mg, 278 μmol, 1 eq.) in DMF (2.0 mL) was added potassium carbonate (77.0 mg, 557 μmol, 2.00 eq.) and 1,2-dibromoethane (105 mg, 557 μmol, 42.0 μL, 2.00 eq.). The mixture was stirred at 80° C. for 2 hours. The reaction mixture was diluted with water (20 mL) and extracted with ethyl acetate (20 mL×3). Combined organic phase was washed with brine (20 mL×3), dried over anhydrous sodium sulfate, filtered and concentrated to give 7-fluoro-6-(4-iodo-2-methyl-pyrazol-3-yl)-2,3-dihydro-1,4-benzodioxine-5-carbonitrile, Intermediate GX (100 mg, 93% yield) as a yellow solid which was used in the next step directly without further purification. LC-MS [M+1]⁺=386.1

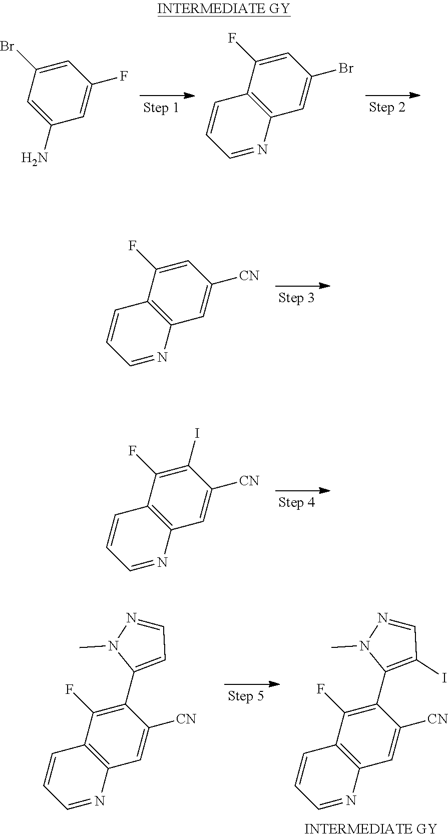

Intermediate GY was prepared following the procedure for Steps 1-5 of Intermediate GU (and Steps 1-2 of Intermediate GM) starting from 3-bromo-5-fluoro-aniline (50.0 g, 263 mmol) to give 5-fluoro-6-(4-iodo-2-methyl-pyrazol-3-yl)quinoline-7-carbonitrile (190 mg, 440 μmol) as brown solid. LCMS [ESI, M+1]⁺378.9. ¹H NMR (400 MHz, CDCl₃) δ=9.23 (dd, J=1.6, 4.4 Hz, 1H), 8.62 (dd, J=1.2, 8.8 Hz, 1H), 8.58 (s, 1H), 7.79-7.72 (m, 2H), 3.88 (s, 3H).

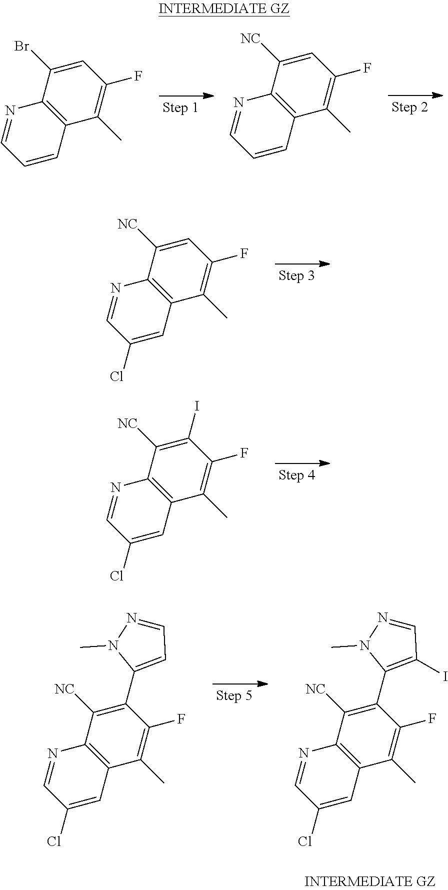

Step 1: A mixture of 5-bromo-7-fluoro-8-methyl-quinoline (37.0 g, 154 mmol, 1.00 eq.), zinc cyanide (21.7 g, 185 mmol, 11.7 mL, 1.20 eq.) and Pd(PPh₃)₄ (17.8 g, 15.4 mmol, 0.10 eq.) in DMF (600 mL) was degassed and stirred at 100° C. for 2 hours under nitrogen atmosphere. The reaction mixture was diluted with water (1.00 L) and extracted with dichloromethane (1.00 L×3). The combined organic layers were washed with brine (2.00 L×2), dried over anhydrous sodium sulfate, filtered and concentrated under reduced pressure and the crude product was triturated with ethyl acetate (300 mL) at 20° C. for 15 min to give 7-fluoro-8-methyl-quinoline-5-carbonitrile (26.0 g, 91% yield) as a white solid. LCMS [M+1]⁺: 187.0. ¹H NMR (400 MHz, CDCl₃) δ=9.08 (dd, J=1.6, 4.0 Hz, 1H), 8.40 (dd, J=1.6, 8.8 Hz, 1H), 7.90 (d, J=8.8 Hz, 1H), 7.61 (dd, J=4.0, 8.8 Hz, 1H), 2.66 (d, J=2.4 Hz, 3H).

Step 2: A mixture of 7-fluoro-8-methyl-quinoline-5-carbonitrile (0.50 g, 2.69 mmol, 1.00 eq.), NCS (1.08 g, 8.06 mmol, 3.00 eq.) in DMF (589 mg, 8.06 mmol, 620 μL, 3.00 eq.) was degassed and stirred at 100° C. for 3 hours under nitrogen atmosphere. The reaction mixture diluted with water (20 mL) and extracted with dichloromethane (30 mL×3). The combined organic layers were washed with brine (100 mL), dried over anhydrous sodium sulfate, filtered and concentrated under reduced pressure and the residue was purified by column chromatography (SiO₂, Ethyl acetate/Petroleum ether 0-20%) to give 3-chloro-7-fluoro-8-methyl-quinoline-5-carbonitrile (300 mg, 51% yield) as a white solid. LCMS [M+1]⁺: 221.0. ¹H NMR (400 MHz, CDCl₃) δ=8.96 (d, J=2.4 Hz, 1H), 8.35 (d, J=2.4 Hz, 1H), 7.88 (d, J=8.8 Hz, 1H), 2.63 (d, J=2.4 Hz, 3H).

Step 3: To a solution of diisopropylamine (7.45 g, 73.65 mmol, 10.41 mL, 1.3 eq.) in THF (200 mL) was added n-butyllithium (2.5 M, 27.19 mL, 1.2 eq.) at −70° C. for 0.5 h. Following that 3-chloro-7-fluoro-8-methyl-quinoline-5-carbonitrile (12.5 g, 56.7 mmol, 1.00 eq.) was added and the mixture was stirred at −40° C. for 1 hour. At that time iodine (15.8 g, 62.3 mmol, 12.5 mL, 1.10 eq.) in THF (150 mL) was added, the mixture was stirred at 25° C. for 1.5 hours. The reaction was quenched by ice water (200 mL) slowly and then extracted with ethyl acetate (100.0 mL×3). The combined organic phase was washed with brine (300.0 mL), dried over anhydrous sodium sulfate, filtered and concentrated in vacuo. The residue was purified by column chromatography (SiO₂, Dichloromethane/Petroleum ether 20-50%) to give 3-chloro-7-fluoro-6-iodo-8-methyl-quinoline-5-carbonitrile (13.3 g, 68% yield) as a white solid. LCMS [M+1]⁺: 346.9. ¹H NMR (400 MHz, CDCl₃) δ=8.90 (d, J=2.0 Hz, 1H), 8.31 (d, J=2.0 Hz, 1H), 2.66 (d, J=2.8 Hz, 3H).

Step 4: A mixture of 3-chloro-7-fluoro-6-iodo-8-methyl-quinoline-5-carbonitrile (2.00 g, 5.77 mmol, 1.00 eq.), 1-methyl-5-(4,4,5,5-tetramethyl-1,3,2-dioxaborolan-2-yl)pyrazole (1.44 g, 6.93 mmol, 1.20 eq.), Pd(dtbpf)Cl₂ (376 mg, 577 μmol, 0.10 eq.) and potassium carbonate (1.20 g, 8.66 mmol, 1.50 eq.) in dioxane (30 mL) and water (6.0 mL) was degassed and stirred at 80° C. for 2 hours under nitrogen atmosphere. The reaction mixture was concentrated under reduced pressure and the residue was purified by column chromatography (SiO₂, Dichloromethane/Petroleum ether 20-35%) to give 3-chloro-7-fluoro-8-methyl-6-(2-methylpyrazol-3-yl)quinoline-5-carbonitrile (0.70 g, 40.3% yield) as a white solid. LCMS [M+1]⁺: 301.1. ¹H NMR (400 MHz, CDCl₃) δ=9.03 (d, J=2.0 Hz, 1H), 8.39 (d, J=2.4 Hz, 1H), 7.69 (d, J=2.0 Hz, 1H), 6.65 (d, J=2.0 Hz, 1H), 3.88 (d, J=1.2 Hz, 3H), 2.70 (d, J=2.4 Hz, 3H).

Step 5: To a solution of 3-chloro-7-fluoro-8-methyl-6-(2-methylpyrazol-3-yl)quinoline-5-carbonitrile (2.00 g, 6.65 mmol, 1.00 eq.) in acetic acid (30 mL) was added NIS (7.48 g, 33.3 mmol, 5.00 eq.). The mixture was stirred at 80° C. for 10 hours. The reaction mixture was concentrated under reduced pressure to remove acetic acid. The residue was quenched by addition saturated sodium bicarbonate solution (50 mL) at 25° C. and extracted with dichloromethane (100 mL×3). The combined organic layers were washed with brine (300 mL×2), dried over anhydrous sodium sulfate, filtered and concentrated under reduced pressure and the crude product was triturated with ethyl acetate (30 mL) at 15° C. for 30 min to give 3-chloro-7-fluoro-6-(4-iodo-2-methyl-pyrazol-3-yl)-8-methyl-quinoline-5-carbonitrile (2.60 g, 91.6% yield) as a yellow solid. LCMS [M+1]⁺: 427.0. ¹H NMR (400 MHz, CDCl₃) δ=9.05 (d, J=2.0 Hz, 1H), 8.42 (d, J=2.0 Hz, 1H), 7.72 (s, 1H), 3.88 (s, 3H), 2.72 (d, J=2.4 Hz, 3H).

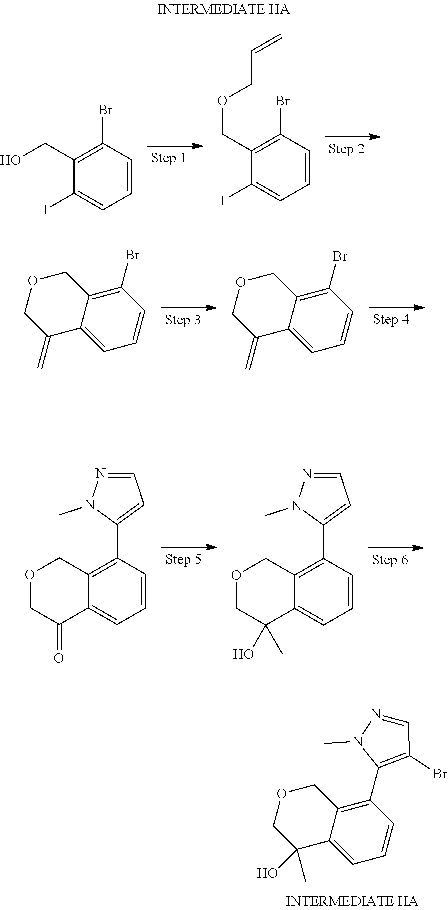

Step 1: To a stirred solution of (2-bromo-6-iodo-phenyl)methanol (2.30 g, 7.35 mmol, 1.00 eq.) in 2-methylTHF (100 mL) was added NaH (882 mg, 22.1 mmol, 60% purity, 3.00 eq.) in portions at 0° C. After being stirred at this temperature for 30 min the mixture was dropwise treated with 3-bromoprop-1-ene (3.56 g, 29.4 mmol, 4.00 eq.), the resulting mixture was stirred at 25° C. for 12 hours. The reaction mixture was quenched with water (20 mL). The mixture was extracted with ethyl acetate (3×30 mL). The combined organic extracts were washed with water (20 mL) and brine (20 mL) and then dried over sodium sulfate, filtered and concentrate under vacuum. The residue was purified by flash silica gel chromatography (Ethyl acetate/Petroleum 1:100) to give 2-(allyloxymethyl)-1-bromo-3-iodo-benzene (2.30 g, 89% yield) as a yellow oil. ¹H NMR (400 MHz, CDCl₃) δ=7.85 (d, J=8.0 Hz, 1H), 7.59 (d, J=8.0 Hz, 1H), 6.84 (t, J=8.0 Hz, 1H), 6.12-5.96 (m, 1H), 5.39 (d, J=17.2 Hz, 1H), 5.25 (d, J=10.4 Hz, 1H), 4.83 (s, 2H), 4.15 (d, J=5.6 Hz, 2H).

Step 2: To a mixture of 2-(allyloxymethyl)-1-bromo-3-iodo-benzene (1.90 g, 5.38 mmol, 1.00 eq.), benzyltrimethylammonium chloride (1.20 g, 6.46 mmol, 1.12 mL, 1.20 eq.), Pd(PPh₃)₄ (311 mg, 269 μmol, 0.05 eq.) and silver carbonate (2.97 g, 10.8 mmol, 488 μL, 2.00 eq.) in N,N-dimethylformamide (60 mL) and water (21.0 mL) was degassed and stirred at 80° C. for 6 hours under nitrogen atmosphere. After being cooled to room temperature, the mixture was concentrated under vacuum. The residue was diluted with ethyl acetate (10.0 mL) and water (20 mL). The layers were separated, and the aqueous phase was extracted with ethyl acetate (3×20 mL). The organic layer was dried over sodium sulfate, filtered and concentrated under vacuum. The residue was purified by flash silica gel chromatography (Petroleum ether) to give 8-bromo-4-methylene-isochromane (1.00 g, 83% yield) as a colorless oil. ¹H NMR (400 MHz, CDCl₃) δ=7.56 (d, J=7.8 Hz, 1H), 7.38 (dd, J=0.8, 7.8 Hz, 1H), 7.05 (t, J=8.0 Hz, 1H), 5.57 (s, 1H), 5.01 (s, 1H), 4.73 (s, 2H) 4.31 (s, 2H).

Step 3: To a solution of 8-bromo-4-methylene-isochromane (1.00 g, 4.44 mmol, 1.00 eq.) in THF (10.0 mL) and water (5.0 mL) was added sodium periodate (2.85 g, 13.3 mmol, 738 μL, 3.00 eq.) and osmium tetroxide (13.0 mg, 51.1 μmol, 2.65 μL, 1.15e-2 eq.). The mixture was stirred at 0° C. for 2 hours. The reaction was quenched with saturated sodium sulfite (10.0 mL). The mixture was extracted with ethyl acetate (3×10.0 mL). The combined organic extracts were washed with saturated brine (20 mL), dried over sodium sulfate and concentrated. The crude product 8-bromoisochroman-4-one (900 mg, 89% yield), a yellow solid, was used directly in the next step without further purification. ¹H NMR (400 MHz, CDCl₃) δ=8.02 (d, J=8.0 Hz, 1H), 7.76 (dd, J=1.2, 8.0 Hz, 1H), 7.31 (t, J=7.8 Hz, 1H), 4.93 (s, 2H), 4.35 (m, 2H).

Step 4: A mixture of 8-bromoisochroman-4-one (900 mg, 3.96 mmol, 1.00 eq.), 1-methyl-5-(4,4,5,5-tetramethyl-1,3,2-dioxaborolan-2-yl)pyrazole (907 mg, 4.36 mmol, 1.10 eq.), sodium bicarbonate (666 mg, 7.93 mmol, 308 μL, 2.00 eq.), (Ad₂n-BuP)—Pd G3 (288 mg, 395 μmol, 9.98e-2 eq.) in dioxane (20 mL) and water (4.0 mL) was degassed and stirred at 80° C. for 12 hours under nitrogen atmosphere. After being cooled to room temperature, the mixture was concentrated under vacuum. The residue was diluted with ethyl acetate (10.0 mL) and water (10.0 mL). The layers were separated and the aqueous phase was extracted with ethyl acetate (3×10.0 mL). The organic layer was dried over sodium sulfate, filtered and concentrated under vacuum. The residue was purified by silica gel chromatography (Ethyl acetate/Petroleum 0-12%) to give 8-(2-methylpyrazol-3-yl)isochroman-4-one (600 mg, 66% yield) as a yellow solid. ¹H NMR (400 MHz, CDCl₃) δ=8.16 (dd, J=2.0, 7.2 Hz, 1H), 7.63-7.47 (m, 3H), 6.24 (d, J=2.0 Hz, 1H), 4.69 (s, 2H), 4.38 (s, 2H), 3.73 (s, 3H).

Step 5: To a solution of 8-(1-methyl-1H-pyrazol-5-yl)isochroman-4-one (300 mg, 1.31 mmol, 1.00 eq.) in 2-methylTHF (5 mL) was added magnesium methyl bromide (3 M, 2.63 mL, 6.00 eq.) at −70° C. The mixture was stirred at 25° C. for 12 hours. The reaction was quenched with saturated ammonium chloride (20 mL). The mixture was extracted with ethyl acetate (3×20 mL). The combined organic extracts were washed with saturated ammonium chloride (10.0 mL) and brine (20 mL), dried over sodium sulfate and concentrated. The residue was purified by flash silica gel chromatography (Ethyl acetate/Petroleum 0-50%) to give 4-methyl-8-(1-methyl-1H-pyrazol-5-yl)isochroman-4-ol (200 mg, 62% yield) as a yellow oil. ¹H NMR (400 MHz, CDCl₃) δ=7.71 (dd, J=1.2, 8.0 Hz, 1H), 7.53 (d, J=2.0 Hz, 1H), 7.39 (t, J=7.60 Hz, 1H), 7.15 (dd, J=1.2, 7.6 Hz, 1H), 6.18 (d, J=2.0 Hz, 1H), 4.52 (d, J=3.2 Hz, 2H), 3.90-3.82 (m, 1H), 3.73 (d, J=11.6 Hz, 1H), 3.68 (s, 3H), 2.50 (s, 1H), 1.58 (s, 3H).

Step 6: To a solution of 4-methyl-8-(2-methylpyrazol-3-yl)isochroman-4-ol (200 mg, 819 μmol, 1.00 eq.) in acetonitrile (10 mL) was added NBS (145 mg, 815 μmol, 0.10 eq.). The mixture was stirred at 25° C. for 12 hours. After being cooled to room temperature, the mixture was concentrated under vacuum. The residue was diluted with ethyl acetate (10.0 mL) and water (10.0 mL). The layers were separated, and the aqueous phase was extracted with ethyl acetate (3×10.0 mL). The organic layer was dried over sodium sulfate, filtered and concentrated under vacuum The residue was purified by silica gel chromatography (Ethyl acetate/Petroleum 0-50%) to give 8-(4-bromo-2-methyl-pyrazol-3-yl)-4-methyl-isochroman-4-ol (240 mg, 91% yield) as a yellow solid. ¹H NMR (400 MHz, CDCl₃) δ=7.76 (d, J=8.0 Hz, 1H), 7.54 (s, 1H), 7.43 (t, J=8.0 Hz, 1H), 7.12 (d, J=7.6 Hz, 1H), 4.65 (d, J=15.6 Hz, 1H), 4.34 (d, J=15.6 Hz, 1H), 3.91 (d, J=11.6 Hz, 1H), 3.75 (d, J=15.6 Hz, 1H), 3.66 (s, 3H), 1.59 (s, 3H).

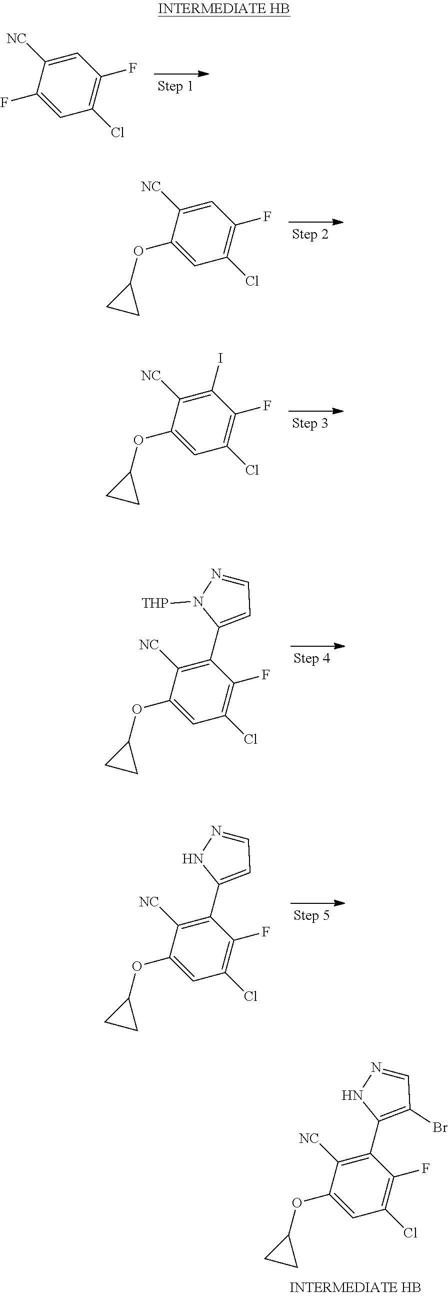

Step 1: To a solution of cyclopropanol (14.3 g, 247 mmol, 1.00 eq.) in THF (400 mL) was added sodium hydride (10.4 g, 259 mmol, 60% purity, 1.05 eq.) at 0° C. After stirring for 30 min, 4-chloro-2,5-difluoro-benzonitrile (42.8 g, 247 mmol, 1.00 eq.) was added to the reaction mixture. The mixture was stirred at 60° C. for 1 hour. The reaction mixture was diluted with water (200 mL) and extracted with ethyl acetate (200 mL×3). Combined organic phase was washed with brine (200 mL), dried over anhydrous sodium sulfate, filtered and concentrated and the residue was purified by column chromatography (SiO₂, Ethyl acetate/Petroleum ether 0-5%) to give 4-chloro-2-(cyclopropoxy)-5-fluoro-benzonitrile (31.5 g, 60% yield) as a white solid. ¹H NMR (400 MHz, CDCl=) δ=7.38 (d, J=6.0 Hz, 1H), 7.34 (d, J=8.0 Hz, 1H), 3.86-3.79 (m, 1H), 0.93-0.88 (m, 4H).

Step 2: To a solution of 4-chloro-2-(cyclopropoxy)-5-fluoro-benzonitrile (6.40 g, 30.2 mmol, 1.00 eq.) in THF (200 mL) was added lithium diisopropylamide (2.00 M, 22.7 mL, 1.50 eq.) at −78° C. After stirring for 0.5 hr, iodine (15.4 g, 60.5 mmol, 12.2 mL, 2.00 eq.) in THF (200 mL) was added to the reaction mixture dropwise. The reaction mixture was stirred at 25° C. for 12 hours. The reaction mixture was quenched with water (200 mL) and extracted with ethyl acetate (200 mL×3). Combined organic phase was washed with brine (200 mL), dried over anhydrous sodium sulfate, filtered and concentrated and the residue was purified by column chromatography (SiO₂, Ethyl acetate/Petroleum ether 0-5%) to give 4-chloro-6-cyclopropoxy-3-fluoro-2-iodobenzonitrile (9.10 g, 89% yield) as a brown solid. ¹H NMR (400 MHz, CDCl₃) δ=7.39 (d, J=6.0 Hz, 1H), 3.87-3.81 (m, 1H), 0.91 (d, J=4.4 Hz, 4H)

Step 3: To a solution of 4-chloro-6-cyclopropoxy-3-fluoro-2-iodobenzonitrile (1.90 g, 5.63 mmol, 1.00 eq.) and 1-tetrahydropyran-2-yl-5-(4,4,5,5-tetramethyl-1,3,2-dioxaborolan-2-yl)pyrazole (1.88 g, 6.76 mmol, 1.20 eq.) in dioxane (20 mL) and water (4.00 mL) was added to sodium bicarbonate (1.42 g, 16.9 mmol, 3.00 eq.) and (Ad₂n-BuP)—Pd G3 (410 mg, 563 μmol, 0.10 eq.) at 20° C., the mixture was stirred at 65° C. for 16 hours under nitrogen. The mixture was extracted with ethyl acetate (80 mL×3), water (60 mL). The combined organic extracts were washed brine (30 mL×3) and then dried over anhydrous sodium sulfate, filtered and concentrated in vacuum and the residue was purified by column chromatography (silicon dioxide, Ethyl acetate/Petroleum ether 10-35%) to give 4-chloro-6-cyclopropoxy-3-fluoro-2-(1-(tetrahydro-2H-pyran-2-yl)-1H-pyrazol-5-yl)benzonitrile (1.65 g, 81% yield) as a yellow solid. ¹H NMR (400 MHz, CDCl₃-d) δ=7.74-7.40 (m, 2H), 6.56-6.25 (m, 1H), 5.44-5.13 (m, 1H), 3.87 (d, J=2.0 Hz, 1H), 3.69 (dd, J=3.2, 12 Hz, 1H), 3.50-3.36 (m, 1H), 2.59-2.39 (m, 1H), 2.13-2.02 (m, 2H), 1.69 (d, J=4.0 Hz, 2H), 1.45-1.06 (m, 1H), 0.93 (s, 4H)

Step 4: To a solution of 4-chloro-6-cyclopropoxy-3-fluoro-2-(1-(tetrahydro-2H-pyran-2-yl)-1H-pyrazol-5-yl)benzonitrile (1.65 g, 4.56 mmol, 1.00 eq.) in methanol (10.0 mL) was added to concentrated hydrochloric acid (12.0 M, 38.0 μL, 0.10 eq.) at 20° C. The mixture was stirred at 20° C. for 1 hour. The reaction mixture was concentrated under reduced pressure and the residue was purified by column chromatography (silicon dioxide, Ethyl acetate/Petroleum ether 20-100%) to give 4-chloro-6-cyclopropoxy-3-fluoro-2-(1H-pyrazol-5-yl)benzonitrile (1.00 g, 3.60 mmol, 79% yield) as a yellow solid. LCMS [M+1]⁺: 278.0. ¹H NMR (400 MHz, CDCl₃d) δ=7.75 (d, J=2.4 Hz, 1H), 7.37 (d, J=6.0 Hz, 1H), 6.83 (t, J=2.8 Hz, 1H), 3.89-3.85 (m, 1H), 1.03-0.80 (m, 4H).

Step 5: To a solution of 4-chloro-6-cyclopropoxy-3-fluoro-2-(1H-pyrazol-5-yl)benzonitrile (300 mg, 1.08 mmol, 1.00 eq.) in dimethylformamide (6.0 mL) was added to N-bromosuccinimide (192 mg, 1.08 mmol, 1.00 eq.) at 15° C. The mixture was stirred at 15° C. for 4 h. The mixture was extracted with ethyl acetate (20 mL×3), water (20 mL). The combined organic extracts were washed brine (20 mL×3) and then dried over anhydrous sodium sulfate, filtered and concentrated in vacuum and the residue was purified by column chromatography (silicon dioxide, Ethyl acetate/Petroleum ether 10-35%) to give 2-(4-bromo-1H-pyrazol-5-yl)-4-chloro-6-cyclopropoxy-3-fluorobenzonitrile (408 mg, crude) as a white solid. LCMS [M+3]⁺: 357.8. ¹H NMR (400 MHz, CDCl₃-d) δ=7.75 (s, 1H), 7.47 (d, J=6.4 Hz, 1H), 4.00-3.77 (m, 1H), 0.96-0.90 (m, 4H).

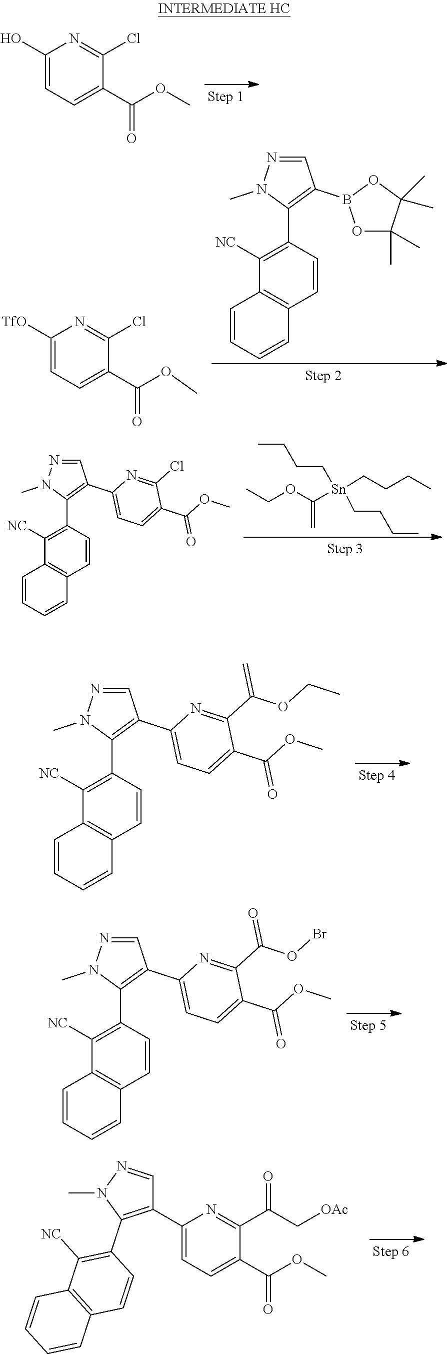

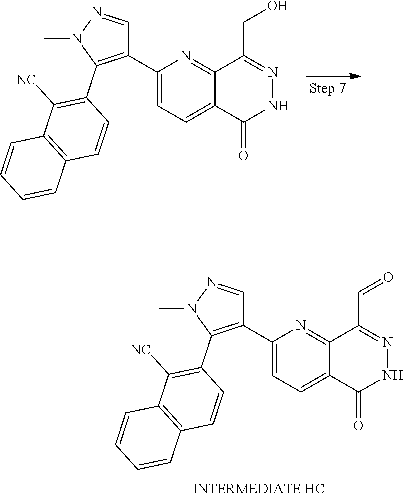 [SMILES: Cn1ncc(-c2ccc3c(=O)[nH]nc(CO)c3n2)c1-c1ccc2ccccc2c1C#N ; Cn1ncc(-c2ccc3c(=O)[nH]nc(C=O)c3n2)c1-c1ccc2ccccc2c1C#N]

Step 1: To a solution of methyl 2-chloro-6-hydroxy-pyridine-3-carboxylate (2.00 g, 10.7 mmol, 1.00 eq.) in DCM (30 mL) was added trifluoromethanesulfonic anhydride (3.61 g, 12.79 mmol, 2.11 mL, 1.20 eq.) and N, N-diisopropylethylamine (2.07 g, 16.0 mmol, 2.79 mL, 1.50 eq.) at 0° C. under nitrogen. Then the mixture was stirred at 20° C. for 1 hour under nitrogen atmosphere. The reaction mixture was concentrated under reduced pressure and the residue was purified by column chromatography (SiO₂, petroleum ether/EtOAc 10/1 to 5/1) to give methyl 2-chloro-6-(trifluoromethylsulfonyloxy) pyridine-3-carboxylate (1.70 g, 5.32 mmol, 50% yield) as a yellow oil. ¹H NMR (400 MHz, CDCl₃) δ=8.39 (d, J=8.0, 1H), 7.21 (d, J=8.0, 1H), 4.00 (s, 3H).

Step 2: To a solution of methyl 2-chloro-6-(trifluoromethylsulfonyloxy)pyridine-3-carboxylate (7.12 g, 22.2 mmol, 1.00 eq.) and 2-[2-methyl-4-(4,4,5,5-tetramethyl-1,3,2-dioxaborolan-2-yl)pyrazol-3-yl]naphthalene-1-carbonitrile (9.60 g, 26.7 mmol, 1.20 eq.) in dioxane (120 mL) and water (10 mL) were added [1,1-bis(diphenylphosphino)ferrocene]dichloropalladium(II) (814.75 mg, 1.11 mmol, 0.05 eq.) and potassium carbonate (6.16 g, 44.5 mmol, 2.00 eq.) at 25° C. under nitrogen. Then the mixture was stirred at 80° C. for 12 hours under nitrogen atmosphere. The reaction mixture was concentrated under reduced pressure to give the crude product. The residue was purified by column chromatography (SiO₂, petroleum ether/EtOAc 10/1 to 2/1) to give methyl 2-chloro-6-[5-(1-cyano-2-naphthyl)-1-methyl-pyrazol-4-yl] pyridine-3-carboxylate (6.00 g, 14.9 mmol, 67% yield) as a light-yellow solid. ¹H NMR (400 MHz, DMSO-d₆) δ=8.47 (d, J=8.4 Hz, 1H), 8.36 (s, 1H), 8.25 (d, J=8.0 Hz, 1H), 8.17 (d, J=8.4 Hz, 1H), 8.13 (d, J=8.0 Hz, 1H), 7.88 (dt, J=1.2, 7.6 Hz, 1H), 7.84-7.79 (m, 1H), 7.78 (d, J=8.4 Hz, 1H), 7.54 (d, J=8.0 Hz, 11H), 3.79 (s, 3H), 3.75 (s, 3H).

Step 3: A mixture of methyl 2-chloro-6-[5-(1-isocyano-2-naphthyl)-1-methyl-pyrazol-4-yl]pyridine-3-carboxylate (6.00 g, 14.9 mmol, 1.00 eq.), tributyl(1-ethoxyvinyl) stannane (8.07 g, 22.3 mmol, 7.54 mL, 1.50 eq.), bis(triphenylphosphine)palladium(II) chloride (1.05 g, 1.50 mmol, 0.10 eq.) in dioxane (80 mL) was degassed and purged with nitrogen for 3 times, and then the mixture was stirred at 80° C. for 12 hours under nitrogen atmosphere. The reaction mixture was diluted with EtOAc (50 mL) and slowly added to saturated aqueous potassium fluoride (50 mL). The mixture was extracted with EtOAc (3×50 mL). The combined organic layers were dried over sodium sulfate, filtered, and concentrated under vacuum. The residue was purified by column chromatography on silica gel (petroleum ether/EtOAc=5/1 to 2/1) to give methyl 2-(1-ethoxyvinyl)-6-[5-(1-isocyano-2-naphthyl)-1-methyl-pyrazol-4-yl] pyridine-3-carboxylate (7.2 g, crude) as a light white solid. ¹H NMR (400 MHz, CDCl₃) δ=8.30 (d, J=8.4 Hz, 1H), 8.13-8.11 (m, 1H), 8.02-7.98 (m, 1H), 7.80-7.74 (m, 2H), 7.72 (dd, J=1.2, 8.0 Hz, 1H), 7.71-7.68 (m, 1H), 7.51 (d, J=8.4 Hz, 1H), 7.23 (d, J=8.0 Hz, 1H), 3.96 (d, J=2.0 Hz, 1H), 3.81-3.79 (m, 6H), 3.76 (d, J=2.0 Hz, 1H), 3.67-3.58 (m, 2H), 1.22-1.18 (m, 3H).

Step 4: To a solution of methyl 2-(1-ethoxyvinyl)-6-[5-(1-isocyano-2-naphthyl)-1-methyl-pyrazol-4-yl] pyridine-3-carboxylate (6.20 g, 14.1 mmol, 1.00 eq.) in tetrahydrofuran (50 mL) was added 1-bromopyrrolidine-2,5-dione (2.52 g, 14.1 mmol, 1.00 eq.) and water (20.0 mL). The mixture was stirred at 15° C. for 12 hours. The mixture was concentrated under vacuum. The residue was diluted with EtOAc (50 mL) and water (50 mL). The layers were separated, and the aqueous phase was extracted with EtOAc (3×50 mL). The organic layers were combined and dried over anhydrous sodium sulfate, filtered and concentrated under vacuum to give methyl 2-(2-bromoacetyl)-6-[5-(1-isocyano-2-naphthyl)-1-methyl-pyrazol-4-yl]pyridine-3-carboxylate (6.90 g, 14.1 mmol, 99% yield) as a yellow solid. ¹H NMR (400 MHz, CDCl₃) δ=8.31 (d, J=8.4 Hz, 1H), 8.23 (d, J=8.4 Hz, 1H), 8.13 (s, 1H), 8.06 (d, J=8.0 Hz, 1H), 7.95 (d, J=8.4 Hz, 1H), 7.85-7.78 (m, 1H), 7.78-7.72 (m, 1H), 7.60 (d, J=8.4 Hz, 1H), 7.52 (d, J=8.4 Hz, 1H), 3.82 (s, 3H), 3.81 (s, 3H), 3.65-3.51 (m, 2H).

Step 5: To a solution of methyl 2-(2-bromoacetyl)-6-[5-(1-isocyano-2-naphthyl)-1-methyl-pyrazol-4-yl] pyridine-3-carboxylate (6.90 g, 14.1 mmol, 1.00 eq.) in DMF (40 mL) and acetonitrile (40 mL) was added sodium acetate (3.47 g, 42.3 mmol, 3.00 eq.). The mixture was stirred at 80° C. for 2 hours. After being cooled to room temperature, the mixture was concentrated under vacuum. The residue was diluted with EtOAc (50 mL) and water (50 mL). The layers were separated, and the aqueous phase was extracted with EtOAc (3×50 mL). The organic layer was dried over anhydrous sodium sulfate, filtered, and concentrated under vacuum. The residue was purified by column chromatography (SiO₂, petroleum ether to petroleum ether:EtOAc=5:1 to 1:1) to give methyl 2-(2-acetoxyacetyl)-6-[5-(1-isocyano-2-naphthyl)-1-methyl-pyrazol-4-yl] pyridine-3-carboxylate (6.5 g, 13.88 mmol, 98% yield) as a white solid. ¹H NMR (400 MHz, CDCl₃) δ=8.30 (d, J=8.4 Hz, 1H), 8.23 (d, J=8.4 Hz, 1H), 8.13 (s, 1H), 8.01 (d, J=8.0 Hz, 1H), 7.88 (d, J=8.4 Hz, 1H), 7.83-7.75 (m, 1H), 7.75-7.67 (m, 1H), 7.56 (d, J=8.4 Hz, 1H), 7.52 (d, J=8.4 Hz, 1H), 4.41-4.24 (m, 2H), 3.82-3.80 (m, 6H), 1.97 (s, 3H).

Step 6: To a solution of methyl 2-(2-acetoxyacetyl)-6-[5-(1-isocyano-2-naphthyl)-1-methyl-pyrazol-4-yl] pyridine-3-carboxylate (6.50 g, 13.9 mmol, 1.00 eq.) in ethanol (50 mL) was added hydrazine hydrate (8.12 g, 159 mmol, 7.89 mL, 11.5 eq.). The mixture was stirred at 75° C. for 5 hours. During this period a white precipitate was formed. It was collected by filtration and dried under high vacuum to give 8-(hydroxymethyl)-2-[5-(1-isocyano-2-naphthyl)-1-methyl-pyrazol-4-yl]-6H-pyrido [2, 3-d]pyridazin-5-one (4.10 g, 10.0 mmol, 72% yield) as a white solid. ¹H NMR (400 MHz, DMSO-d₆) δ=12.61 (s, 1H), 8.52 (s, 1H), 8.50 (d, J=8.6 Hz, 1H), 8.46 (d, J=8.6 Hz, 1H), 8.27 (d, J=8.0 Hz, 1H), 8.16 (d, J=8.4 Hz, 1H), 8.08 (d, J=8.6 Hz, 1H), 7.93-7.82 (m, 2H), 7.80 (d, J=8.6 Hz, 1H), 4.24 (t, J=6.4 Hz, 1H), 3.74 (s, 3H), 3.70-3.55 (m, 2H).

Step 7: To a solution of 8-(hydroxymethyl)-2-[5-(1-isocyano-2-naphthyl)-1-methyl-pyrazol-4-yl]-6H-pyrido [2,3-d]pyridazin-5-one (1.00 g, 2.45 mmol, 1.00 eq.) in DMF (20 mL) was added manganese dioxide (4.26 g, 49.0 mmol, 20.0 eq.). The mixture was stirred at 80° C. for 24 hours. The solid was then removed by filtration through a pad of celite and washed with DCM followed by DMF. The filtrate was concentrated under vacuum. The residue was purified by prep-TLC (SiO₂, DCM:methanol=10:1) to give 2-[5-(1-isocyano-2-naphthyl)-1-methyl-pyrazol-4-yl]-5-oxo-6H-pyrido [2,3-d]pyridazine-8-carbaldehyde (300 mg, 738 μmol, 30% yield) as a white solid. ¹H NMR (400 MHz, DMSO-d₆) δ=12.77 (s, 1H), 8.53-8.45 (m, 3H), 8.31-8.24 (m, 1H), 8.16 (d, J=8.6 Hz, 1H), 8.17-8.12 (m, 1H), 7.91-7.75 (m, 4H), 3.76-3.71 (m, 3H).

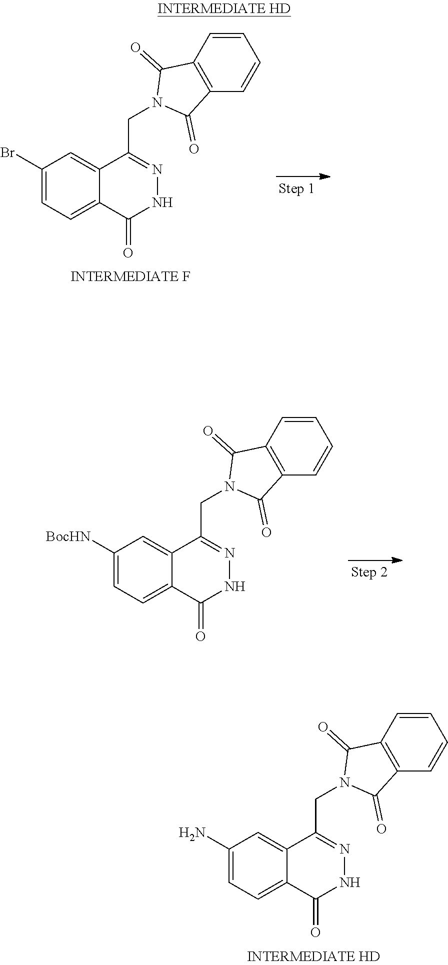 [SMILES: O=C1c2ccccc2C(=O)N1Cc1n[nH]c(=O)c2ccc(Br)cc12 ; CC(C)(C)OC(=O)Nc1ccc2c(=O)[nH]nc(CN3C(=O)c4ccccc4C3=O)c2c1 ; Nc1ccc2c(=O)[nH]nc(CN3C(=O)c4ccccc4C3=O)c2c1]

Step 1: A mixture of 2-[(7-bromo-4-oxo-3H-phthalazin-1-yl)methyl]isoindoline-1,3-dione (1.50 g, 3.90 mmol, 1.0 eq.), tert-butyl carbamate (686 mg, 5.86 mmol, 1.50 eq.), cesium carbonate (2.54 g, 7.81 mmol, 2.00 eq.), Pd(OAc)₂ (88 mg, 390 μmol, 0.1 eq.) and Xantphos (226 mg, 390 μmol, 0.1 eq.) in dioxane (50 mL) was degassed and purged with nitrogen 3 times, and then the mixture was stirred at 100° C. for 2 hours under a nitrogen atmosphere. The mixture was concentrated under reduced pressure to give a residue and the residue was tritiated with EtOAc (20 mL) for 30 minutes to give tert-butyl N-[4-[(1,3-dioxoisoindolin-2-yl)methyl]-1-oxo-2H-phthalazin-6-yl]carbamate (1.10 g, crude) as a yellow solid. The crude product was used for the next step without further purification. LCMS [M+1]⁺=421.2.

Step 2: To a solution of tert-butyl N-[4-[(1,3-dioxoisoindolin-2-yl)methyl]-1-oxo-2H-phthalazin-6-yl]carbamate (1.00 g, 2.38 mmol, 1.00 eq.) was added HCl/MeOH (4 M, 30 mL). The mixture was stirred at 20° C. for 1 hour. The mixture was then concentrated under reduced pressure to give a residue. The crude product was purified by reversed-phase flash chromatography (0.1% formic acid conditions) to give 2-[(7-amino-4-oxo-3H-phthalazin-1-yl)methyl]isoindoline-1,3-dione (100 mg, 312 μmol, two steps 13% yield) as a yellow solid. LCMS [M+1]⁺=321.1.

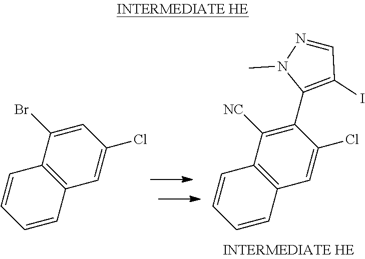 [SMILES: Clc1cc(Br)c2ccccc2c1 ; Cn1ncc(I)c1-c1c(Cl)cc2ccccc2c1C#N]

Intermediate HE was prepared following the same procedure as for Steps 2-5 of Intermediate GU starting from 1-bromo-3-chloro-naphthalene (7.30 g, 30.2 mmol) to give 8-3-chloro-2-(2-methylpyrazol-3-yl)naphthalene-1-carbonitrile (1.50 g, 5.60 mmol) as a yellow solid. ¹H NMR (400 MHz, DMSO-d₆) δ=8.71 (s, 1H), 8.19 (d, J=8.4, 11.6 Hz, 2H), 7.95-7.80 (m, 2H) 7.64 (d, J=1.8 Hz, 1H), 6.60 (d, J=1.8 Hz 1H) 3.69 (s, 3H).

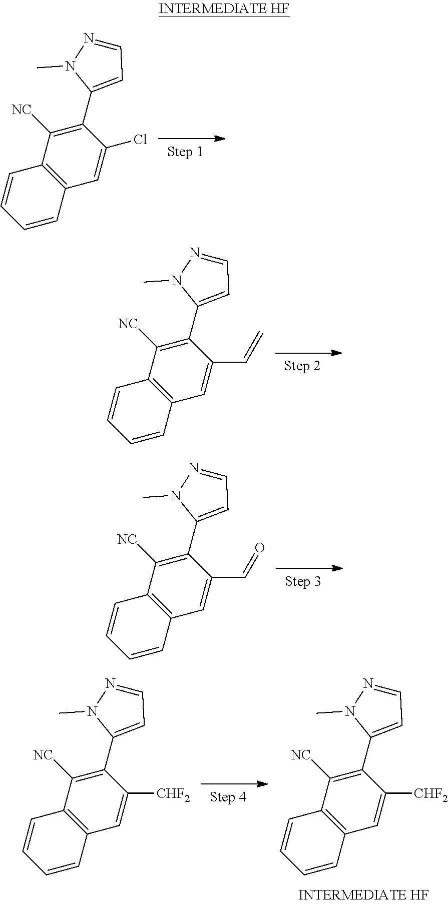

Step 1: To a solution of 3-chloro-2-(2-methylpyrazol-3-yl)naphthalene-1-carbonitrile (500 mg, 1.87 mmol, 1.00 eq) and potassium difluoro(vinyl)borane fluoride (300 mg, 2.24 mmol, 1.20 eq) in tetrahydrofuran (5 mL) and water (0.5 mL) was added dichloropalladium (33 mg, 187 μmol, 0.10 eq) and dicyclohexyl-[2-(2,6-diisopropoxyphenyl)phenyl]phosphane (87 mg, 187 μmol, 0.10 eq) and cesium carbonate (1.83 g, 5.60 mmol, 3.00 eq). The mixture was stirred at 90° C. for 16 hours. The solution was poured into water (100 mL) and extracted with EtOAc (150 mL×3). The combined organic layer was concentrated under vacuum to give the crude product. The residue was purified by flash silica gel choursomatography (0-100% EtOAc/Petroleum ether) to give 2-(2-methylpyrazol-3-yl)-3-vinyl-naphthalene-1-carbonitrile (480 mg, 1.85 mmol, 99% yield) as a yellow solid. ¹HNMR (400 MHz, DMSO-d₆) δ=8.71 (s, 1H), 8.22 (d, J=8.0 Hz, 1H), 8.14 (d, J=8.4 Hz, 1H), 7.88-7.76 (m, 2H), 7.65 (d, J=1.6 Hz, 1H), 6.55 (d, J=1.6 Hz, 1H), 6.36 (dd, J=12.0, 18.0 Hz, 1H), 5.98 (d, J=18.0 Hz, 1H), 5.45 (d, J=12.0 Hz, 1H), 3.58 (s, 3H).

Step 2: To a solution of 2-(2-methylpyrazol-3-yl)-3-vinyl-naphthalene-1-carbonitrile (450 mg, 1.74 mmol, 1.00 eq) in tetrahydrofuran (15 mL) and water (15 mL) were added dipotassium osmium tetraoxide dihydrate (32 mg, 86 μmol, 0.05 eq) and sodium periodate (1.48 g, 6.94 mmol, 4.00 eq). The mixture was stirred at 20° C. for 3 hours. The solution was poured into water (10 mL) and extracted with EtOAc (30 mL×3). The combined organic layer was concentrated and the residue purified by flash silica gel choursomatography (0-100% EtOAc/Petroleum ether) to give 3-formyl-2-(2-methylpyrazol-3-yl)naphthalene-1-carbonitrile (450 mg, 1.72 mmol, 99% yield) as a yellow solid. ¹HNMR (400 MHz, DMSO-d₆) δ=8.17-8.08 (m, 2H), 8.02-7.93 (m, 2H), 7.76-7.64 (m, 2H).

Step 3: To a solution of 3-formyl-2-(2-methylpyrazol-3-yl)naphthalene-1-carbonitrile (400 mg, 1.53 mmol, 1.00 eq) in dichloromethane (10 mL) was added 2-methoxy-N-(2-methoxyethyl)-N-(trifluoro-k-sulfanyl)ethanamine (847 mg, 3.83 mmol, 2.50 eq.). The mixture was stirred at 20° C. for 6 hours. The solution was poured into water (10 mL) and extracted with dichloromethane (30 mL×3), the combined organic layer was concentrated under vacuum and purified by flash silica gel choursomatography (0-100% EtOAc/Petroleum ether) to give 3-(difluoromethyl)-2-(2-methylpyrazol-3-yl)naphthalene-1-carbonitrile (400 mg, 1.41 mmol, 92% yield) as a yellow solid. ¹HNMR (400 MHz, DMSO-d₆) δ=8.78 (s, 1H), 8.40 (d, J=8.2 Hz, 1H), 8.24 (d, J=8.0 Hz, 1H), 8.03-7.97 (m, 1H), 7.94-7.87 (m, 1H), 7.65 (d, J=1.9 Hz, 1H), 6.56 (d, J=1.9 Hz, 1H), 3.64 (s, 3H).

Step 4: To a solution of 3-(difluoromethyl)-2-(2-methylpyrazol-3-yl)naphthalene-1-carbonitrile (300 mg, 1.06 mmol, 1.00 eq) in acetonitrile (4 mL) was added N-iodosuccinimide (595 mg, 2.65 mmol, 2.50 eq.) and acetic acid (530 umol, 30 uL, 0.50 eq). The mixture was stirred at 80° C. for 6 hours. The solution was poured into water (10 mL) and extracted with EtOAc (30 mL×3) and the combined organic layers were concentrated under vacuum to give the crude product. The residue was purified by flash silica gel choursomatography (0-100% EtOAc/Petroleum ether) to give 3-(difluoromethyl)-2-(4-iodo-2-methyl-pyrazol-3-yl)naphthalene-1-carbonitrile (400 mg, 978 μmol, 92% yield) was obtained as a yellow solid. ¹H NMR (400 MHz, DMSO-d₆) δ=8.85 (s, 1H), 8.42 (d, J=8.1 Hz, 1H), 8.26 (d, J=8.3 Hz, 1H), 8.07-7.91 (m, 2H), 7.80 (s, 1H), 7.04-6.67 (m, 1H), 3.69 (s, 3H).

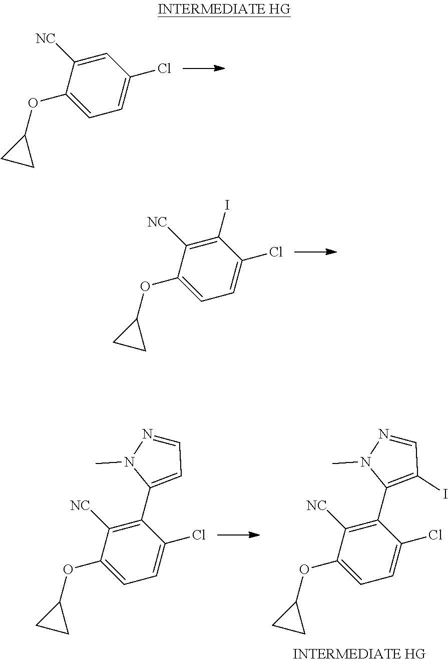 [SMILES: N#Cc1cc(Cl)ccc1OC1CC1 ; N#Cc1c(OC2CC2)ccc(Cl)c1I ; Cn1nccc1-c1c(Cl)ccc(OC2CC2)c1C#N ; Cn1ncc(I)c1-c1c(Cl)ccc(OC2CC2)c1C#N]

3-chloro-6-(cyclopropoxy)-2-(4-iodo-2-methyl-pyrazol-3-yl)benzonitrile, intermediate HG, was prepared as a yellow solid (160 mg, 400 μmol), following the procedure described for the preparation of Intermediate DQ. LCMS [M+1]⁺=399.9; ¹H NMR (400 MHz, CDCl₃) δ=7.70 (d, J=9.2 Hz, 1H), 7.64 (s, 1H), 7.47 (d, J=9.2 Hz, 1H), 3.92 (td, J=2.8, 5.6 Hz, 1H), 0.99-0.90 (m, 4H).

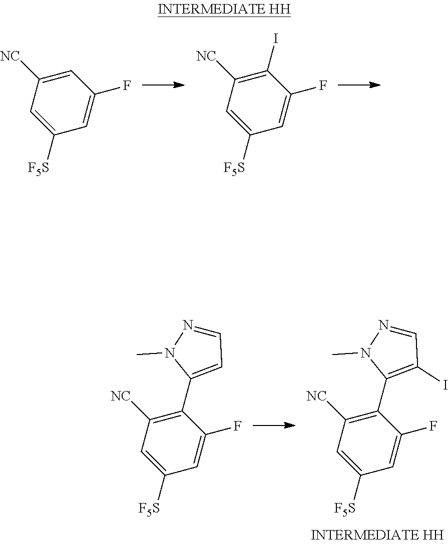

3-fluoro-2-(4-iodo-2-methyl-pyrazol-3-yl)-5-(pentafluoro-λ⁶-sulfanyl)benzonitrile, intermediate HH, was prepared as a yellow solid (260 mg, 574 μmol), following the procedure described for the preparation of Intermediate DQ. LCMS [M+1]⁺=454.0; ¹H NMR (400 MHz, CDCl₃) δ=8.06 (s, 1H) 7.92 (dd, J=8.8, 2.0 Hz, 1H) 7.71 (s, 1H) 3.86 (s, 3H).

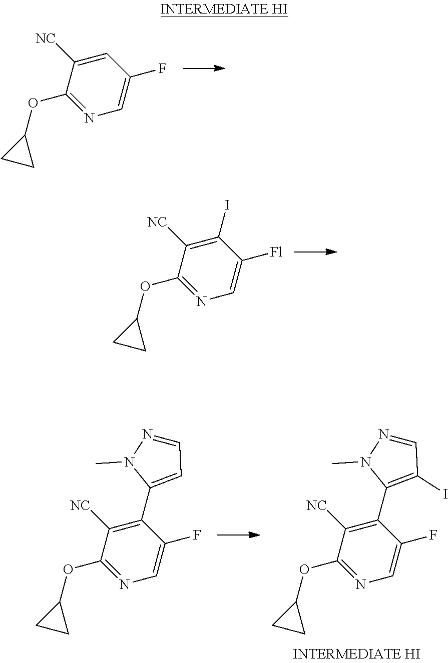

2-(cyclopropoxy)-5-fluoro-4-(2-methylpyrazol-3-yl)pyridine-3-carbonitrile, intermediate HI, was prepared as a yellow solid (170 mg, 658 μmol), following the procedure described for the preparation of Intermediate DQ. LCMS [M+1]⁺=259.0; ¹H NMR (400 MHz, CDCl₃) δ=8.38 (s, 1H), 7.64 (d, J=2.0 Hz, 1H), 6.61 (d, J=2.0 Hz, 1H), 4.42 (dq, J=3.2, 6.4 Hz, 1H), 3.86 (d, J=1.6 Hz, 3H), 0.91-0.87 (m, 4H).

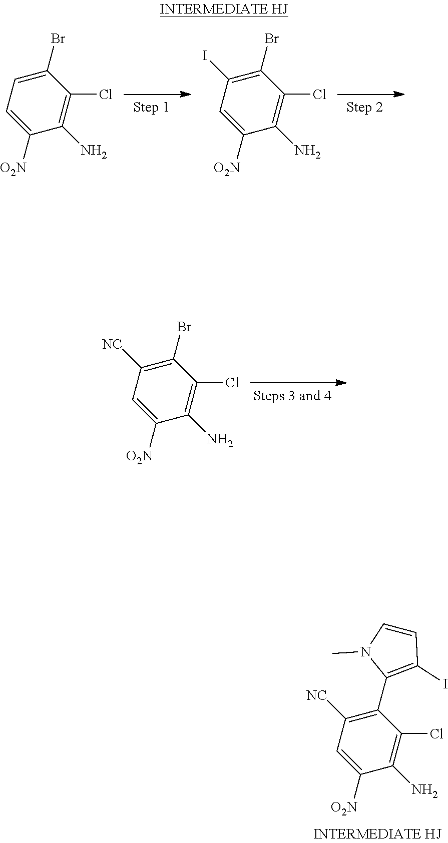 [SMILES: Nc1c([N+](=O)[O-])ccc(Br)c1Cl ; Nc1c([N+](=O)[O-])cc(I)c(Br)c1Cl ; N#Cc1cc([N+](=O)[O-])c(N)c(Cl)c1Br ; Cn1ccc(I)c1-c1c(C#N)cc([N+](=O)[O-])c(N)c1Cl]

Step 1: To a solution of 3-bromo-2-chloro-6-nitro-aniline (3.70 g, 14.7 mmol) in acetic acid (20 mL) was added 1-iodopyrrolidine-2,5-dione (6.62 g, 29.4 mmol, 2.00 eq.) and stirred at 70° C. for 3 hours. The reaction mixture was diluted with EtOAc (10 mL) and very slowly added to a saturated aqueous sodium bicarbonate (20 mL). The mixture was extracted with EtOAc (3×20 mL). The combined organic layers were dried over anhydrous sodium sulfate, filtered and concentrated under vacuum to give 3-bromo-2-chloro-4-iodo-6-nitro-aniline (3.6 g, 9.54 mmol, 65% yield) as a yellow solid. ¹H NMR (400 MHz, DMSO-d₆) δ=8.41 (s, 1H), 7.56 (s, 2H).

Step 2: To a solution of 3-bromo-2-chloro-4-iodo-6-nitro-aniline (3.00 g, 7.95 mmol) in dimethylsulfoxide (30 mL) was added cuprous cyanide (854 mg, 9.54 mmol, 1.2 eq.). The mixture was stirred at 90° C. for 12 hours then cooled to room temperature and concentrated. The residue was diluted with water (30 mL) and the mixture extracted with EtOAc (3×50 mL). The combined organic layers were dried over anhydrous sodium sulfate, filtered and concentrated. The residue was purified by column chromatography on silica gel (petroleum ether/EtOAc=10/1 to 5/1) to give 3-bromo-2-chloro-4-isocyano-6-nitro-aniline (1.97 g, 7.13 mmol, 89.6% yield) as a yellow solid. ¹H NMR (400 MHz, CDCl₃-d) δ=8.47 (s, 1H).

Step 3 and 4: 2-chloro-3-(4-iodo-2-methyl-pyrazol-3-yl)-4-isocyano-6-nitro-aniline, intermediate HJ, was prepared as a yellow solid (100 mg, 247 μmol) as a yellow solid following the procedure described for the preparation of Intermediate DQ. ¹H NMR (400 MHz, CDCl₃-d) δ=8.60 (s, 1H), 7.67 (s, 1H), 3.80 (s, 3H).

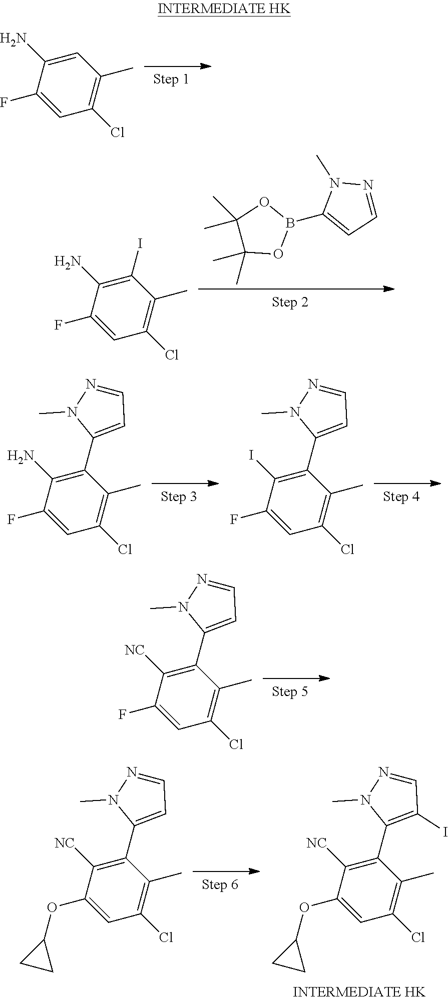

Step 1: To a solution of 4-chloro-2-fluoro-5-methyl-aniline (4.90 g, 30.7 mmol) in EtOH (450 mL) was added silver sulfate (10.1 g, 32.2 mmol, 1.05 eq.) followed by addition of iodine (8.18 g, 32.2 mmol, 1.05 eq.) in portions. After the addition was completed, the mixture was stirred at 20° C. for 2 hours. The mixture was filtered, and the filtrate was evaporated to give a dark oil which was dissolved in EtOAc (125 mL). The solution was washed with 2M sodium hydroxide (40 mL×2), saturated sodium thiosulfate (40 mL×2) and water (40 mL×2). The resulting solution was dried over anhydrous sodium sulfate and then concentrated under reduced pressure and the residue was purified by column chromatography (SiO₂, petroleum ether/EtOAc 30/1 to 5/1) to give 4-chloro-6-fluoro-2-iodo-3-methyl-aniline (7.20 g, 25.2 mmol, 82% yield) as a white solid LCMS [M+1]⁺=286.0; ¹H NMR (400 MHz, CDCl₃) δ=7.08 (d, J=10.39 Hz, 1H), 4.19 (br s, 2H), 2.53 (s, 3H).

Step 2: A mixture of 4-chloro-6-fluoro-2-iodo-3-methyl-aniline (6.00 g, 21.0 mmol), 1-methyl-5-(4,4,5,5-tetramethyl-1,3,2-dioxaborolan-2-yl)pyrazole (6.56 g, 31.5 mmol, 1.5 eq.), Pd(dtbpf)Cl₂ (1.37 g, 2.10 mmol, 0.1 eq.), sodium carbonate (6.68 g, 63.1 mmol, 3.0 eq.) in dioxane (60 mL) and water (12 mL) was degassed and purged with nitrogen for 3 times, and then the mixture was stirred at 80° C. for 5 hours under nitrogen atmosphere. The mixture was concentrated under reduced pressure and the residue purified by column chromatography (SiO₂, petroleum ether/EtOAc 20/1 to 2/1) to give 4-chloro-6-fluoro-3-methyl-2-(2-methylpyrazol-3-yl) aniline (3.00 g, 12.5 mmol, 59% yield) as a white solid. LCMS [M+1]⁺=240.2; ¹H NMR (400 MHz, CDCl₃) δ=7.61 (d, J=1.88 Hz, 1H) 7.12 (d, J=10.51 Hz, 1H) 6.23 (d, J=1.75 Hz, 1H) 3.14-3.98 (m, 5H) 1.99 (s, 3H).

Step 3: To a solution of p-methylbenzene sulfonic acid (9.34 g, 54.2 mmol, 5.00 eq.) in acetonitrile (50 mL) was added 4-chloro-6-fluoro-3-methyl-2-(2-methylpyrazol-3-yl) aniline (2.60 g, 10.9 mmol). The mixture was cooled to 0° C. and to this was added a solution of sodium nitrite (1.50 g, 21.7 mmol, 2.0 eq.) and potassium iodide (5.40 g, 32.5 mmol, 3.0 eq.) in water (500 mL). The dark suspension was stirred at 0-5° C. for 10 minutes and then at 20° C. for 1 hour. The mixture was concentrated under reduced pressure and the residue was purified by column chromatography (SiO₂, petroleum ether/EtOAc 20/1 to 5/1) to give 5-(5-chloro-3-fluoro-2-iodo-6-methyl-phenyl)-1-methyl-pyrazole (1.85 g, 5.28 mmol, 42% yield) as a yellow solid. LCMS [M+1]⁺=351.0; ¹H NMR (400 MHz, CDCl₃) δ=7.55-7.62 (m, 1H) 7.19-7.26 (m, 1H) 6.12-6.18 (m, 1H) 3.59 (t, J=2.51 Hz, 3H) 2.11-2.14 (m, 3H).

Step 4: A mixture of 5-(5-chloro-3-fluoro-2-iodo-6-methyl-phenyl)-1-methyl-pyrazole (1.80 g, 5.13 mmol), zinc cyanide (362 mg, 3.08 mmol, 0.6 eq.), Pd(PPh₃)₄ (593 mg, 513 μmol, 0.1 eq.) in DMF (15 mL) was degassed and purged with nitrogen for 3 times, and then the mixture was stirred at 100° C. for 12 hours under nitrogen atmosphere. The mixture was cooled to 25° C., diluted with EtOAc (100 mL) and filtered. The filtrate was washed with brine (50 mL×3). The organic phase was dried over anhydrous sodium sulfate filtered and concentrated under reduced pressure and the residue was purified by column chromatography (SiO₂, petroleum ether/EtOAc 20/1 to 1/1) to give 4-chloro-6-fluoro-3-methyl-2-(2-methylpyrazol-3-yl)benzonitrile (850 mg, 3.40 mmol, 66% yield) as a yellow solid. LCMS [M+1]⁺=250.1; ¹H NMR (400 MHz, CDCl₃) δ=7.60 (d, J=1.88 Hz, 1H) 7.40 (d, J=8.25 Hz, 1H) 6.34 (d, J=1.88 Hz, 1H) 3.68 (s, 3H) 2.14 (d, J=0.75 Hz, 3H).

Step 5: To a solution of cyclopropanol (209 mg, 3.60 mmol, 1.20 eq.) in THF (8.00 mL) was added NaH (144 mg, 3.60 mmol, 60% purity, 1.20 eq.) at 0° C. The mixture was stirred at 0° C. for 0.5 hour. Then 4-chloro-6-fluoro-3-methyl-2-(2-methylpyrazol-3-yl)benzonitrile (750 mg, 3.00 mmol, 1.00 eq.) was added to the mixture and the result mixture was stirred at 60° C. for 1 hour. The reaction mixture was diluted with water (50 mL) and extracted with EtOAc (50 mL×3). The combined organic phase was washed with brine 50 mL, dried over anhydrous sodium sulfate, filtered and concentrated to give a residue. The residue was purified by column chromatography (SiO₂, petroleum ether/EtOAc 10/1 to 1/1) to give 4-chloro-6-(cyclopropoxy)-3-methyl-2-(2-methylpyrazol-3-yl)benzonitrile (700 mg, 2.43 mmol, 81% yield) as a yellow solid. LCMS [M+1]⁺=288.2; ¹H NMR (400 MHz, CDCl₃) δ=7.58 (d, J=1.88 Hz, 1H) 7.47 (s, 1H) 6.29 (d, J=1.88 Hz, 1H) 3.80-3.92 (m, 1H) 3.66 (s, 3H) 2.07-2.09 (m, 3H) 0.90 (d, J=5.00 Hz, 4H).

Step 6: To a solution of 4-chloro-6-(cyclopropoxy)-3-methyl-2-(2-methylpyrazol-3-yl)benzonitrile (600 mg, 2.09 mmol) in acetic acid (10 mL) was added NIS (938 mg, 4.17 mmol, 2.00 eq.). The mixture was stirred at 80° C. for 0.5 hour. The mixture was concentrated under reduced pressure and the residue was purified by column chromatography (SiO₂, petroleum ether/EtOAc=10/1 to 1/1) to give 4-chloro-6-(cyclopropoxy)-2-(4-iodo-2-methyl-pyrazol-3-yl)-3-methyl-benzonitrile (650 mg, 1.57 mmol, 75% yield) as a white solid. LCMS [M+1]⁺=414.1; ¹H NMR (400 MHz, CDCl₃) δ=7.62 (s, 1H) 7.52 (s, 1H) 3.84-3.91 (m, 1H) 3.71-3.73 (m, 3H) 2.07 (s, 3H) 0.89-0.96 (m, 4H).

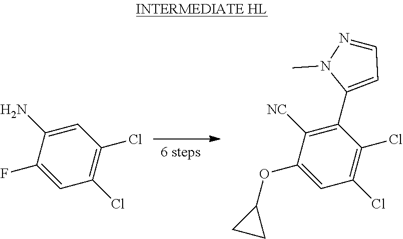 [SMILES: Nc1cc(Cl)c(Cl)cc1F ; Cn1nccc1-c1c(Cl)c(Cl)cc(OC2CC2)c1C#N]

INTERMEDIATE HL, 3,4-dichloro-6-(cyclopropoxy)-2-(2-methylpyrazol-3-yl)benzonitrile (80 mg, 260 μmol) was prepared as a white solid in an analogs 6-step procedure to INTERMEDIATE HK. LCMS [M+1]⁺=433.8; ¹H NMR (400 MHz, CDCl₃-d) δ=7.64 (s, 1H), 7.27-7.26 (m, 1H), 3.97-3.87 (m, 1H), 3.78 (s, 2H), 1.01-0.94 (m, 3H).

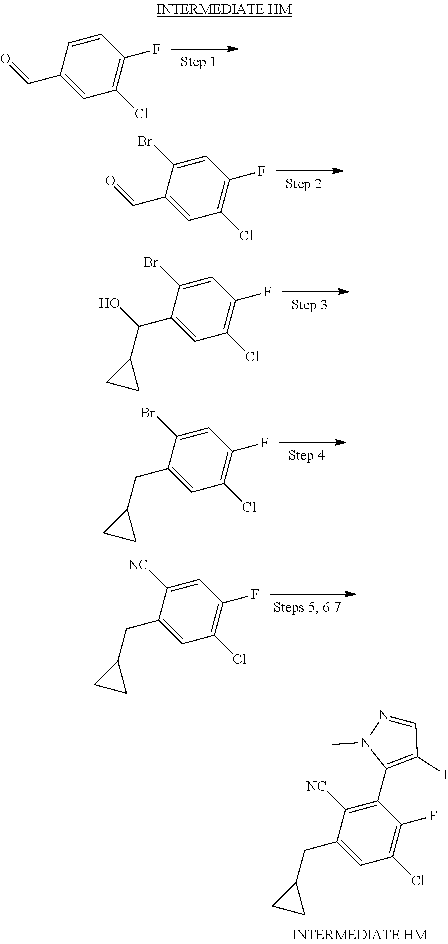

Step 1: To a solution of 3-chloro-4-fluoro-benzaldehyde (10.0 g, 63.1 mmol) in 1, 2-dichloroethane (30 mL) and TFA (6 mL) were added 4-chloro-2-(trifluoromethyl) aniline (2.47 g, 12.6 mmol, 1.77 mL, 0.20 eq.), palladium acetate (1.42 g, 6.31 mmol, 0.10 eq.) and N-bromosuccinimide (12.4 g, 69.4 mmol, 1.10 eq.) at 20° C. Then the reaction mixture was stirred at 60° C. for 12 hours. The mixture was diluted with water (80 mL) and extracted with EtOAc (80 mL×3). The combined organic extracts were washed with brine (20 mL×3) and then dried over anhydrous sodium sulfate, filtered and concentrated in vacuum and the residue was purified by flash silica gel chromatography (0-60% EtOAc/Petroleum) to give 2-bromo-5-chloro-4-fluoro-benzaldehyde (1.80 g, 7.58 mmol, 12% yield) as a yellow solid. ¹H NMR (400 MHz, CDCl₃-d) δ=10.22 (s, 1H), 7.99 (d, J=8.0 Hz, 1H), 7.47 (d, J=8.0 Hz, 1H).

Step 2: To a solution of 2-bromo-5-chloro-4-fluoro-benzaldehyde (1.60 g, 6.74 mmol) in 2-methyltetrahydrofuran (10 mL) was added bromo(cyclopropyl)magnesium (0.5 M, 13.5 mL, 1.00 eq.) at −70° C. Then the reaction mixture was stirred at 20° C. for 3 hours under nitrogen. The mixture was diluted with water (30 mL) and extracted with ethyl acetate (30 mL×3). The combined organic phase was dried with anhydrous sodium sulfate, filtered and concentrated in vacuum to give a residue. The residue was purified by column chromatography (SiO₂, petroleum ether/ethyl acetate 1/0 to 10/1) to give (2-bromo-5-chloro-4-fluoro-phenyl)-cyclopropyl-methanol (1.5.0 g, 5.37 mmol, 79% yield) as a white solid.

Step 3: To a solution of (2-bromo-5-chloro-4-fluoro-phenyl)-cyclopropyl-methanol (800 mg, 2.86 mmol, 1.00 eq.) in dichloromethane (5 mL) were added triethyl silicane (499 mg, 4.29 mmol, 686 μL, 1.50 eq.) and TFA (1.96 g, 17.2 mmol, 1.27 mL, 6.00 eq.) at 0° C. Then the reaction mixture was stirred at 40° C. for 2 hours. The mixture was diluted with water (30 mL) and extracted with ethyl acetate (30 mL×3). The combined organic extracts were washed brine (20 mL×3) and dried over anhydrous sodium sulfate, filtered and concentrated in vacuum to give a residue. The residue was purified by column chromatography (SiO₂, petroleum ether/ethyl acetate 1/0 to 10/1) to give 1-bromo-4-chloro-2-(cyclopropylmethyl)-5-fluoro-benzene (500 mg, 1.90 mmol, 66% yield) as a colorless liquid.

Step 4: To a solution of 1-bromo-4-chloro-2-(cyclopropylmethyl)-5-fluoro-benzene (500 mg, 1.90 mmol) in DMF (5 mL) were added zinc cyanide (111 mg, 949 μmol, 0.50 eq.) and tetrakis[triphenylphosphine]palladium (219 mg, 190 μmol, 0.10 eq.) at 20° C. The reaction mixture was degassed and purged with nitrogen for 3 times, and then the mixture was stirred at 110° C. for 4 hours under nitrogen atmosphere. The reaction mixture was adjusted to pH 11 by sodium hydroxide solution (1 N), and quenched by addition sodium hypochlorite solution (5 mL) at 25° C. The mixture was diluted with water (30 mL) and extracted with ethyl acetate (30 mL×3). The combined organic layers were washed with brine (20 mL×3), dried over anhydrous sodium sulfate, filtered and concentrated under reduced pressure to give a residue. The residue was purified by column chromatography (SiO₂, petroleum ether/EtOac 1/0 to 20/1) to give 1-chloro-5-(cyclopropylmethyl)-2-fluoro-4-isocyano-benzene (150 mg, 715 μmol, 37% yield) as a yellow oil. ¹H NMR (400 MHz, CDCl₃-d) δ=7.47 (d, J=7.2 Hz, 1H), 7.32 (d, J=8.4 Hz, 1H), 2.66 (d, J=7.2 Hz, 2H), 0.95 (m, 1H), 0.62-0.49 (m, 2H), 0.22 (q, J=4.8 Hz, 2H).

Step 5, 6 and 7: INTERMEDIATE HM, 4-(aminomethyl)-6-[5-[3-chloro-5-(cyclopropylmethyl)-2-fluoro-6-isocyano-phenyl]-1-methyl-pyrazol-4-yl]-2H-phthalazin-1-one (13 mg, 27 μmol, 91% yield, hydrochloride) was prepared as a white solid starting from 1-chloro-5-(cyclopropylmethyl)-2-fluoro-4-isocyano-benzene in a analogous 3-step procedure to that which was used for the preparation of INTERMEDIATE DQ. LCMS [M+1]⁺=461.3; ¹H NMR (400 MHz, DMSO-d₆) δ=12.87 (s, 1H) 8.46-8.60 (m, 3H) 8.38-8.45 (m, 1H) 8.13 (d, J=8.31 Hz, 1H) 7.87 (s, 1H) 7.78 (s, 1H) 4.30-4.45 (m, 2H) 4.23 (br d, J=2.32 Hz, 1H) 3.61-3.70 (m, 3H) 2.02 (s, 3H) 0.79-0.95 (m, 4H).

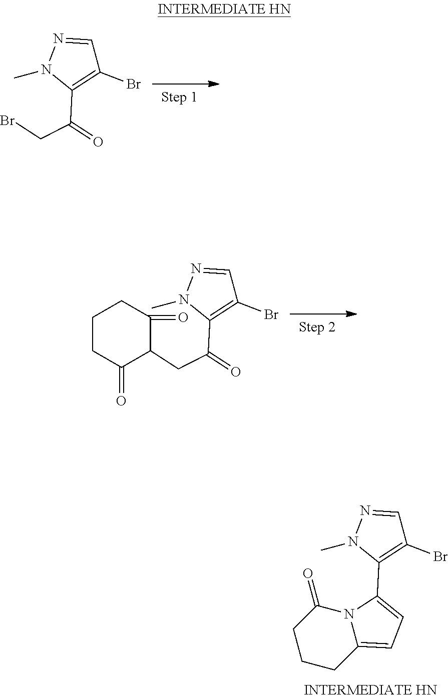 [SMILES: Cn1ncc(Br)c1C(=O)CBr ; Cn1ncc(Br)c1C(=O)CC1C(=O)CCCC1=O ; Cn1ncc(Br)c1-c1ccc2n1C(=O)CCC2]

Step 1: To a solution of 2-bromo-1-(4-bromo-2-methyl-pyrazol-3-yl)ethanone (1.30 g, 4.61 mmol) and cyclohexane-1,3-dione (620 mg, 5.53 mmol, 1.20 eq.) in DMF (20 mL) was added potassium carbonate (1.27 g, 9.22 mmol, 2.00 eq.). The mixture was stirred at 25° C. for 2 hours. The mixture was filtered, concentrated and the residue was purified by flash silica gel chromatography (80-100% ethyl acetate/petroleum ether) to give 2-[2-(4-bromo-2-methyl-pyrazol-3-yl)-2-oxo-ethyl]cyclohexane-1,3-dione (350 mg, 849 μmol, 18% yield) as a yellow solid. LCMS [ESI, M+1]⁺=312.9; ¹H NMR (400 MHz, CD₃OD-d₆) δ=7.53 (s, 1H), 4.01 (s, 3H), 3.98-3.79 (m, 3H), 2.47 (t, J=6.4 Hz, 4H), 2.06-1.97 (m, 2H).

Step 2: A mixture of 2-[2-(4-bromo-2-methyl-pyrazol-3-yl)-2-oxo-ethyl] cyclohexane-1,3-dione (300 mg, 958 μmol, 1.00 eq.) and ammonium acetate (1.48 g, 19.2 mmol, 20.0 eq.) in acetic acid (10 mL) was stirred at 140° C. for 12 hours. The mixture was adjusted to pH=8-9 with aq sodium carbonate and extracted with ethyl acetate (30 mL×3). The combined organic layers were washed with brine, dried over sodium sulfate, filtered and concentrated. The residue was purified by flash silica gel chromatography (60-80% ethyl acetate/petroleum ether) to give -(4-bromo-2-methyl-pyrazol-3-yl)-7,8-dihydro-6H-indolizin-5-one (100 mg, 323 μmol, 33% yield) as a light-yellow solid. ¹H NMR (400 MHz, CDCl₃) δ=8.75 (br s, 1H), 7.05 (s, 1H), 6.55 (d, J=2.4 Hz, 1H), 3.75 (s, 3H), 2.70 (t, J=6.0 Hz, 2H), 2.42-2.27 (m, 2H), 2.02 (m, J=6.4 Hz, 2H). LCMS [ESI, M+1]⁺=294.1.

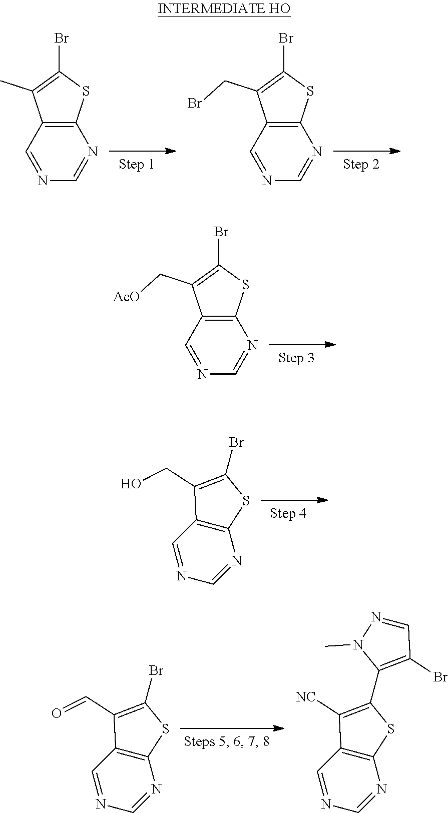 [SMILES: Cc1c(Br)sc2ncncc12 ; BrCc1c(Br)sc2ncncc12 ; CC(=O)OCc1c(Br)sc2ncncc12 ; OCc1c(Br)sc2ncncc12 ; O=Cc1c(Br)sc2ncncc12 ; Cn1ncc(Br)c1-c1sc2ncncc2c1C#N]

Step 1: To a cooled solution of trichloromethane (80 mL) were added 6-bromo-5-methyl-thieno[2,3-d]pyrimidine (2.20 g, 9.60 mmol, 1.00 eq.), N-bromosuccinimide (2.05 g, 11.5 mmol, 1.20 eq.) and benzoyl peroxide (233 mg, 960 μmol, 0.10 eq.) at 0° C. Then the reaction mixture was allowed to warm to 20° C. for 16 hours. The residue was extracted with dichloromethane (80 mL×3), water (60 mL), the combined organic extracts were washed brine (30 mL×3) and then dried over anhydrous sodium sulfate, filtered and concentrated in vacuum to give 6-bromo-5-(bromomethyl)thieno[2,3-d]py-rimidine (2.10 g, 6.82 mmol, 71% yield) as a brown solid. LCMS [M+1]⁺: 308.6; ¹H NMR (400 MHz, MeOD-d₄) δ=9.20 (s, 1H), 9.07 (s, 1H), 4.86 (s, 2H).

Step 2: To a solution of 6-bromo-5-(bromomethyl)thieno[2,3-d]pyrimidine (600 mg, 1.95 mmol, 1.00 eq.) in dimethylformamide DMF (10 mL) was added sodium acetate (479 mg, 5.84 mmol, 3.00 eq.) at 20° C. Then the mixture was then stirred at 80° C. for 4 hours. Water (40 mL) was added and the mixture was extracted with ethyl acetate (30 mL×3). The combined organic extracts were washed brine (10 mL×3) and then dried over anhydrous sodium sulfate, filtered and concentrated in vacuum to give a residue. The residue was purified by prep-TLC (SiO₂, petroleum ether/ethyl acetate=3/1) to give (6-bromothieno[2,3-d]pyrimidin-5-yl)methyl acetate (258 mg, 899 μmol, 46% yield) as a yellow solid. LCMS [M+1]⁺=286.8; ¹H NMR (400 MHz, CDCl₃) δ=9.18 (s, 1H), 9.09 (s, 1H), 5.36 (s, 2H), 2.11 (s, 3H).

Step 3: To a solution of (6-bromothieno[2,3-d]pyrimidin-5-yl)methyl acetate (250 mg, 871 μmol, 1.00 eq.) in THF (5 mL) and water (0.5 mL) was added to lithium hydroxide monohydrate (110 mg, 2.61 mmol, 3.00 eq.) at 20° C. for 2 hours. The residue was extracted with ethyl acetate (30 mL×3), water (20 mL). The combined organic extracts were washed brine (10 mL×3) and then dried over anhydrous sodium sulfate, filtered and concentrated in vacuum to give (6-bromothieno[2,3-d]pyrimidin-5-yl)methanol (180 mg, 734 μmol, 84% yield) as yellow solid. LCMS [M+1]⁺=247.1; ¹H NMR (400 MHz, CDCl₃-d) δ=9.33 (s, 1H), 9.07 (s, 1H), 4.98 (s, 2H).

Step 4: To a solution of (6-bromothieno[2,3-d]pyrimidin-5-yl)methanol (180 mg, 734 μmol, 1.00 eq.) in dichloromethane (10 mL) was added Dess-Martin periodinane (467 mg, 1.10 mmol, 1.50 eq.) at 20° C. The reaction mixture was stirred at 20° C. for 6 hours. The mixture was diluted with dichloromethane (20 mL), washed with saturated aqueous sodium bicarbonate (10 mL×3), brine (10 mL), dried over sodium sulfate, filtered and concentrated to give the crude product. The residue was purified by column chromatography (SiO₂, dichloromethane/methanol 20/1 to 3/1) to give 6-bromothieno[2,3-d]pyrimidine-5-carbaldehyde (160 mg, 658 μmol, 89% yield) as yellow solid. LCMS [M+1]⁺: 245.0; ¹H NMR (400 MHz, CDCl₃-d) δ=10.19 (s, 1H), 9.86 (s, 1H), 9.15 (s, 1H).

Steps 5, 6, 7 and 8: 4-(aminomethyl)-6-[5-(5-isocyanothieno[2,3-d]pyrimidin-6-yl)-1-methyl-pyrazol-4-yl]-2H-phthalazin-1-one (3 mg, 6.2 μmol, hydrochloride) was prepared as a white solid from 6-bromothieno[2,3-d]pyrimidine-5-carbaldehyde in an analogous procedure to that which INTERMEDIATE DC was prepared. LCMS [M+1]⁺: 415.1; ¹H NMR (400 MHz, MeOD-d₄) δ=9.41 (s, 1H), 9.28 (s, 1H), 8.26 (d, J=8.4 Hz, 1H), 8.17 (s, 1H), 7.94 (s, 1H), 7.65 (d, J=8.4 Hz, 1H), 7.51-7.32 (m, 2H), 4.50 (s, 2H), 4.03 (s, 3H).

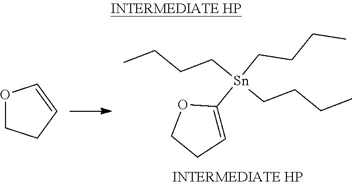 [SMILES: C1=COCC1 ; CCC[CH2][Sn]([CH2]CCC)([CH2]CCC)[C]1=CCCO1]

A solution of 2,3-dihydrofuran (5.0 g, 71.3 mmol, 5.4 mL, 1.0 eq.) in tetrahydrofuran (8.0 mL) was cooled to −60° C. Then a solution of tertiary butyl lithium (1.3 M in heptane, 71.3 mL, 1.3 eq.) was added dropwise. The yellow solution was stirred at −60° C. for 10 minutes then at 0° C. for 50 minutes. The reaction mixture was cooled down to −60° C. and tributylchlorostannane (27.3 g, 83.8 mmol, 22.6 mL, 1.18 eq.) was added dropwise to give a colorless solution that was stirred at 0° C. for 90 minutes. The reaction mixture was poured slowly into saturated aqueous ammonium chloride and the resulting mixture was extracted with ethyl acetate (3-80 mL). The combined organic layers were washed with brine (100 mL), dried over sodium sulfate, filtered and concentrated under reduced pressure to give a residue. The crude product was distilled in vacuum (140° C., −0.09 MPa) to give tributyl(4,5-dihydrofuran-2-yl)stannane (25.0 g, 69.6 mmol, 97% yield) as a colorless oil. ¹H NMR (400 MHz, CHLOROFORM-d) δ=5.06 (t, J=2.4 Hz, 1H), 4.23 (t, J=9.6 Hz, 2H), 2.62-2.50 (m, 2H), 1.70-1.60 (m, 3H), 1.40-1.28 (m, 12H), 0.93-0.88 (m, 12H).

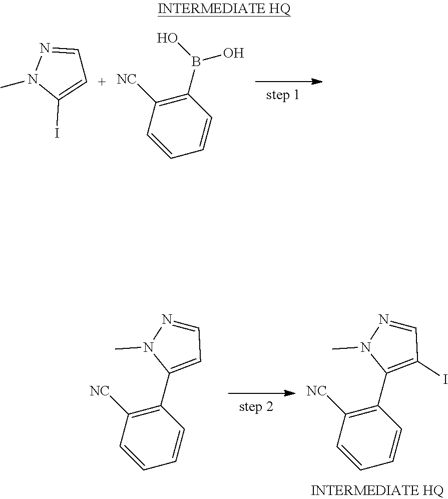

Step 1: A solution of 5-iodo-1-methyl-1H-pyrazole (500 mg, 2.40 mmol, 1.0 eq.), (2-cyanophenyl)boronic acid (706 mg, 4.81 mmol, 2.0 eq.), potassium phosphate (1.5 M, 4.01 mL, 2.5 eq.), cataCXium Pd G3 (175 mg, 240 μmol, 0.1 eq.) in toluene (10.0 mL) was degassed and stirred at 90° C. for 4 hours under nitrogen atmosphere. The mixture was diluted with ethyl acetate (150 mL) and water (100 mL). The organic layer was dried over sodium sulfate, filtered and concentrated under vacuum. The crude product was purified by column chromatography on silica gel (petroleum ether/ethyl acetate 3-50%) to give 2-(1-methyl-1H-pyrazol-5-yl)benzonitrile (450 mg, 2.36 mmol, 98% yield) as a yellow solid. LCMS [M+H]⁺: 183.8.

Step 2: To a solution of 2-(1-methyl-1H-pyrazol-5-yl)benzonitrile (390 mg, 2.13 mmol, 1.0 eq.) in acetic acid (5.0 mL) was added N-Iodosuccinimide (958 mg, 4.26 mmol, 2.0 eq.). The mixture was stirred at 20° C. for 1 hour. The mixture was diluted with ethyl acetate (50 mL) and water (20 mL). The organic layer was dried over sodium sulfate, filtered and concentrated under vacuum. The residue was purified by column chromatography on silica gel (petroleum ether/ethyl acetate 3-50%) to give 2-(4-iodo-1-methyl-1H-pyrazol-5-yl)benzonitrile, Intermediate HQ (545 mg, 1.76 mmol, 83% yield) as a brown solid. LCMS [M+H]⁺: 309.7. ¹H NMR (400 MHz, chloroform-d) δ=7.86 (d, J=8.0 Hz, 1H), 7.79-7.74 (m, 1H), 7.66-7.60 (m, 2H), 7.47 (d, J=7.6 Hz, 1H), 3.84 (s, 3H);

The following Examples are intended to illustrate further certain embodiments of the invention and are not intended to limit the scope of the invention.

### General Reaction Methods for the Preparation of Examples 1-1 to 1-8

### Examples 1-1 and 1-2

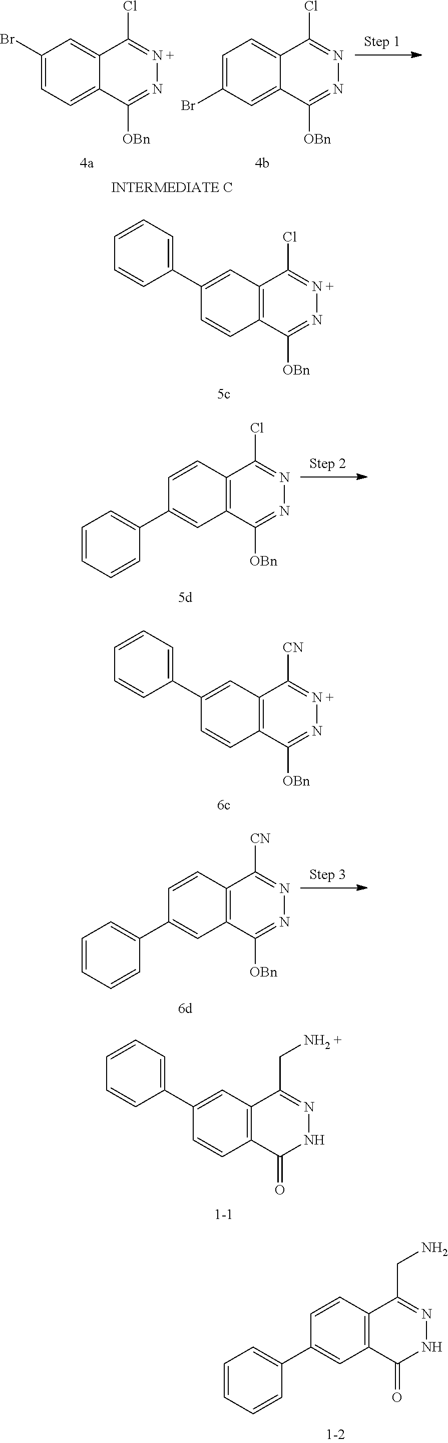

Step 1: A mixture of Intermediate C, a 1:1 mixture of 4c and 4d (587 mg, 1.68 mmol, 1.00 eq.), phenylboronic acid pinacol ester, Pd(dppf)Cl₂ (168 μmol, 0.10 eq.) and sodium carbonate (3.36 mmol, 2.00 eq.) in DMF (10 mL) was purged with nitrogen 3 times and stirred at 100° C. for 2 hours. After such time the reaction mixture was diluted with water (50 mL) and extracted with ethyl acetate (50 mL×3). The combined organic layers were washed with brine (50 mL), dried over anhydrous sodium sulfate, filtered and concentrated under reduced pressure. The residue was purified by prep-TLC (SiO₂, Petroleum ether/Ethyl acetate 5:1) to give a 1:1 mixture of 4-(benzyloxy)-1-chloro-7-phenylphthalazine 5c and 5d 4-benzyloxy-1-chloro-6-phenyl-phthalazine (355 mg, 818 μmol, 48% yield) as a yellow solid.

Step 2: To a 1:1 mixture of 4-(benzyloxy)-1-chloro-6-phenylphthalazine 5c and 4-(benzyloxy)-1-chloro-7-phenylphthalazine 5d (350 mg, 806 μmol, 1.00 eq.) in DMF (10 mL) was added zinc cyanide (1.21 mmol, 1.50 eq.), 1,1′-bis(diphenylphosphino)ferrocene (80.6 μmol, 0.10 eq.), Pd₂(dba) (40.3 μmol, 0.05 eq.) and zinc powder (80.6 μmol, 0.10 eq.). The mixture was purged with nitrogen 3 times and stirred at 100° C. for 3 hours. After such time the reaction mixture was diluted with water (50 mL) and extracted with ethyl acetate (50 mL×3). The combined organic layers were washed with brine (50 mL), dried over anhydrous sodium sulfate, filtered and concentrated under reduced pressure to a residue. The residue was purified by column chromatography (SiO₂, Ethyl acetate/Petroleum ether 10-35%) to give a 1:1 mixture of 4-(benzyloxy)-7-phenylphthalazine-1-carbonitrile 6c and 4-benzyloxy-6-phenyl-phthalazine-1-carbonitrile 6d (158 mg, 468 μmol, 58% yield) as a yellow solid.

Step 3: To a 1:1 mixture of 4-(benzyloxy)-7-phenylphthalazine-1-carbonitrile 6c and 4-(benzyloxy)-6,6-phenylphthalazine-1-carbonitrile 6d (100 mg, 296 μmol, 1.00 eq.) was added hydrochloric acid (6.00 M, 10.1 eq.) and methyl alcohol (3.0 mL) followed by palladium on activated carbon (29.6 μmol, 10% by mass) under nitrogen. The suspension was degassed under vacuum and purged with hydrogen several times. The mixture was then stirred under a hydrogen atmosphere (50 psi) at 40° C. for 2 hours. After such time the reaction mixture was filtered, concentrated under reduced pressure and the residue purified by prep-HPLC (Phenomenex Luna C18 150×25 mm×10 μm; mobile phase: [water (0.1% TFA)-ACN]; B %: 2%-25%, 10 min) followed by separation of the regioisomers by SFC (column: DAICEL CHIRALPAK AD (250 mm×30 mm, 10 μm); mobile phase: [0.1% NH₃H₂O MeOH]; B %: 60/6-60%, 40 min] to give the desired compounds 4-(aminomethyl)-6-phenyl-phthalazin-1-ol, Example 1-1 (13.7 mg, 53.6 μmol, 26% yield) LCMS [M+1]⁺=252.2; ¹H NMR (400 MHz, MeOD) δ=8.37 (d, J=8.0 Hz, 1H), 8.09-8.02 (m, 2H), 7.76 (d, J=7.2 Hz, 2H), 7.54-7.48 (m, 2H), 7.47-7.41 (m, 1H), 4.22 (s, 2H). LCMS [M+1]: 252.2 and 4-(aminomethyl)-7-phenyl-phthalazin-1-ol, Example 1-2 (23.6 mg, 91.8 μmol, 46% yield) LCMS [M+1]: 252.3; ¹H NMR (400 MHz, MeOD) δ=8.64 (d, J=2.0 Hz, 1H), 8.28 (dd, J=2.0, 8.4 Hz, 1H), 8.01 (d, J=8.4 Hz, 1H), 7.83-7.74 (d, J=7.2 Hz, 2H), 7.58-7.51 (m, 2H), 7.50-7.44 (m, 1H), 4.61 (s, 2H).

Following the teachings of the General Reaction Schemes, the synthesis procedure for Examples 1-1 & 1-2 and using the Intermediates disclosed herein, the Examples 1-3 to 1-8 were prepared as shown in Table 1:

TABLE 1

| Example | Structure | Compound Name and Characterization |
|---|---|---|
| 1-3 | 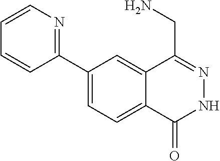 [SMILES: NCc1n[nH]c(=O)c2ccc(-c3ccccn3)cc12] | 4-(aminomethyl)-6-(pyridin-2-yl)phthalazin-1(2H)-one LCMS [M + 1]⁺ = 253.2; ¹H NMR (400 MHz, MeOD) δ = 8.76-8.72 (m, 1H), 8.55 (s, 1H), 8.50-8.43 (m, 2H), 8.14-8.09 (m, 1H), 8.00 (dt, J = 2.0, 7.6 Hz, 1H), 7.49 (ddd, J = 0.8, 5.2, 7.6 Hz, 1H), 4.29 (br s, 2H) |
| 1-4 | 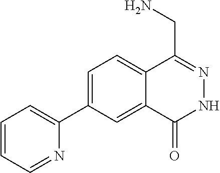 [SMILES: NCc1n[nH]c(=O)c2cc(-c3ccccn3)ccc12] | 4-(aminomethyl)-7-(pyridin-2-yl)phthalazin-1(2H)-one LCMS [M + 1]⁺ = 253.2; ¹H NMR (400 MHz, MeOD) δ = 8.96 (d, J = 1.6 Hz, 1H), 8.74-8.70 (m, 1H), 8.60 (dd, J = 2.0, 8.4 Hz, 1H), 8.12-8.05 (m, 2H), 8.02-7.95 (m, 1H), 7.46 (ddd, J = 1.2, 4.8, 7.6 Hz, 1H), 4.28 (s, 2H). |
| 1-5 | 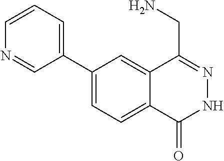 [SMILES: NCc1n[nH]c(=O)c2ccc(-c3cccnc3)cc12] | 4-(aminomethyl)-6-(pyridin-3-yl)phthalazin-1(2H)-one LCMS [M + 1]⁺ = 253.2; ¹H NMR (400 MHz, MeOD) δ = 8.91 (dd, J = 0.8, 2.4 Hz, 1H), 8.54 (dd, J = 1.6, 4.8 Hz, 1H), 8.39 (d, J = 8.4 Hz, 1H), 8.24-8.18 (m, 1H), 8.13 (d, J = 1.2 Hz, 1H), 8.09 (dd, J = 1.6, 8.4 Hz, 1H), 7.52 (ddd, J = 0.8, 4.8, 8.0 Hz, 1H), 4.24 (s, 2H). |
| 1-6 | 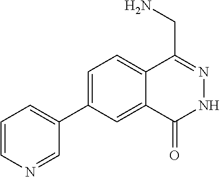 [SMILES: NCc1n[nH]c(=O)c2cc(-c3cccnc3)ccc12] | 4-(aminomethyl)-7-(pyridin-3-yl)phthalazin-1(2H)-one LCMS [M + 1]⁺ = 253.2; ¹H NMR (400 MHz, MeOD) δ = 8.95 (d, J = 1.6 Hz, 1H), 8.66-8.60 (m, 2H), 8.32- 8.22 (m, 2H), 8.11 (d, J = 8.4 Hz, 1H), 7.60 (ddd, J = 0.8, 4.8, 8.0 Hz, 1H), 4.23 (s, 2H). |
| 1-7 | 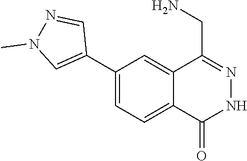 [SMILES: Cn1cc(-c2ccc3c(=O)[nH]nc(CN)c3c2)cn1] | 4-(aminomethyl)-6-(1-methyl-1H-pyrazol-4-yl)phthal- azin-1(2H)-one LCMS [M + 1]⁺ = 256.2; ¹H NMR (400 MHz, MeOD) δ = 8.38 (d, J = 8.4 Hz, 1H), 8.28 (s, 1H), 8.16-8.10 (m, 2H), 8.05 (s, 1H), 4.64 (s, 2H), 3.99 (s, 3H) |
| 1-8 | 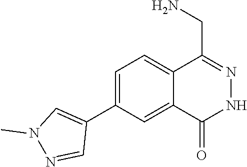 [SMILES: Cn1cc(-c2ccc3c(CN)n[nH]c(=O)c3c2)cn1] | 4-(aminomethyl)-7-(1-methyl-1H-pyrazol-4-yl)phthal- azin-1(2H)-one LCMS [M + 1]⁺ = 256.2; ¹H NMR (400 MHz, MeOD) δ = 8.50 (d, J = 2.0 Hz, 1H), 8.23 (s, 1H), 8.18 (dd, J = 2.0, 8.4 Hz, 1H), 8.02 (s, 1H), 7.91 (d, J = 8.4 Hz, 1H), 4.57 (s, 2H), 3.98 (s, 3H). |

### Example 2-1

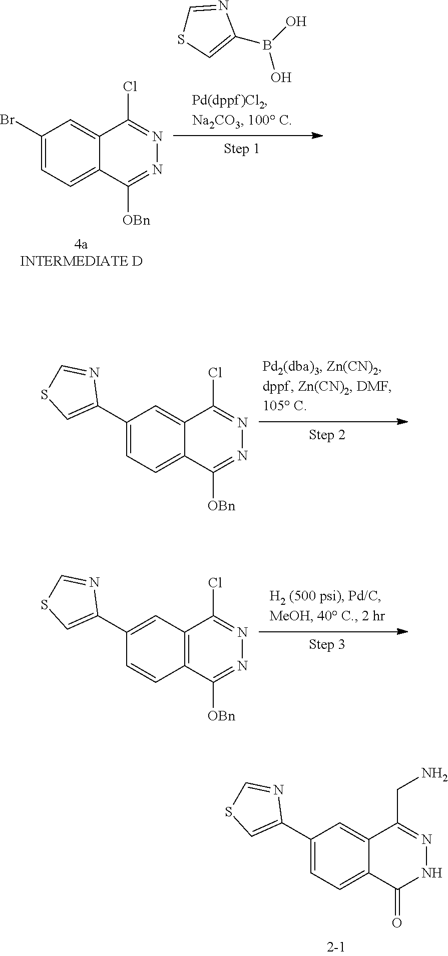 [SMILES: OB(O)c1cscn1 ; Clc1nnc(OCc2ccccc2)c2ccc(Br)cc12 ; Clc1nnc(OCc2ccccc2)c2ccc(-c3cscn3)cc12 ; Clc1nnc(OCc2ccccc2)c2ccc(-c3cscn3)cc12 ; NCc1n[nH]c(=O)c2ccc(-c3cscn3)cc12]

Step 1: A mixture of Intermediate D (150 mg, 429 μmol, 1.00 eq.), thiazol-4-ylboronic acid (66 mg, 515 μmol, 1.20 eq.), sodium carbonate (91 mg, 858 μmol, 2.00 eq.) and Pd(dppf)Cl₂ (31 mg, 42.9 μmol, 0.10 eq.) in DMF (1.0 mL) was purged with nitrogen 3 times and stirred at 100° C. for 12 hours under nitrogen. After such time the reaction mixture was diluted with water (20 mL) and extracted with ethyl acetate (20 mL×3). The combined organic layers were washed with brine (30 mL), dried over sodium sulfate, filtered and concentrated under reduced pressure to a residue. The residue was then purified by column chromatography (SiO₂, petroleum ether:ethyl acetate 5:1 to 1:1) to give 4-(1-(benzyloxy)-4-chlorophthalazin-6-yl)thiazole (76 mg, 214 μmol, 50% yield).

Step 2: A mixture of 4-(1-(benzyloxy)-4-chlorophthalazin-6-yl)thiazole (76 mg, 214 μmol, 1.00 eq.), zinc cyanide (38 mg, 321 μmol, 20 μL, 1.50 eq.), DPPF (12 mg, 21 μmol, 0.10 eq.), Pd₂(dba)₃ (10 mg, 10 μmol, 0.05 eq.) and zinc powder (1 mg, 21 μmol, 0.10 eq.) in DMF (1.0 mL) was degassed and purged with nitrogen 3 times. The mixture was then stirred at 105° C. for 2 hours under a nitrogen atmosphere. After such time the reaction mixture was diluted with water (20 mL), extracted with ethyl acetate (20 mL×3) and the combined organic layers washed with brine (30 mL), dried (Na₂SO₄ anhydrous), filtered and concentrated under reduced pressure. The residue was purified by column chromatography (SiO₂, petroleum ether:ethyl acetate=5:1 to 1:1) to give 4-(benzyloxy)-7-(thiazol-4-yl)phthalazine-1-carbonitrile (51 mg, 148 μmol, 69% yield).

Step 3: To a solution of 4-(benzyloxy)-7-(thiazol-4-yl)phthalazine-1-carbonitrile (103 mg, 299 μmol, 1.00 eq), HCl (6.0 M, 1.00 eq.) in MeOH (10 mL) was added palladium on activated carbon (296 μmol, 10.0% purity) under nitrogen. The suspension was degassed under vacuum and purged with hydrogen several times. The mixture was then stirred vigorously in a hydrogen atmosphere (50.0 psi) at 40° C. for 2 hours. After such time the reaction mixture was filtered, concentrated under reduced pressure and purified by prep-HPLC (Waters Xbridge 150×25 mm×5 μm; mobile phase: [water (0.05% ammonia hydroxide v/v)-ACN]; B %: 12/6-42%, 10 min) to furnish 4-(aminomethyl)-6-(thiazol-4-yl)phthalazin-1 (2H)-one, Example 2-1 (3 mg, 12 μmol, 4% yield, 97% purity) as a white solid. LCMS [M+1]: 259.2; ¹H NMR (400 MHz, MeOD) δ=9.17 (d, J=2.0 Hz, 1H), 8.56 (s, 1H), 8.47-8.43 (m, 2H), 8.33 (d, J=2.0 Hz, 1H), 4.29 (s, 2H).

### General Reaction Method 2 for the Preparation of Examples 2-2 to 2-5

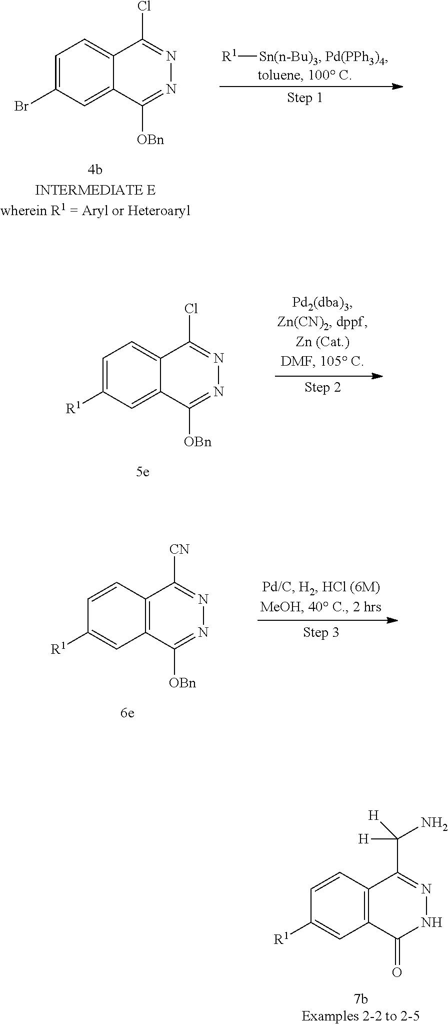

Step 1: A mixture of Intermediate E (429 μmol, 1.00 eq.), the appropriate aryl/heteroaryl-tributyltin reagent (644 μmol, 1.50 eq) and Pd(PPh₃)₄ (43 μmol, 0.10 eq.) in toluene (2 mL) was purged with nitrogen 3 times and stirred at 100° C. for 12 hours. After such time the mixture was diluted with water (20 mL) and extracted with ethyl acetate (20 mL×3). The combined organic layers were washed with brine (30 mL), dried over anhydrous sodium sulfate, filtered and concentrated under reduced pressure to a residue. The residue was then purified by column chromatography (SiO₂, petroleum ether:ethyl acetate 5:1 to 1:1) to give appropriate R¹-coupled product 5e.

Step 2: A mixture of the R¹-coupled product 5e (214 μmol, 1.00 eq.), zinc cyanide (321 μmol, 20 μL, 1.50 eq.), DPPF (21 μmol, 0.10 eq.), Pd₂(dba)₃ (10 μmol, 0.05 eq.) and zinc powder (21 μmol, 0.10 eq.) in DMF (1.0 mL) was purged with nitrogen 3 times. The mixture was then stirred at 105° C. for 2 hours. After such time the mixture was diluted with water (20 mL) and extracted with ethyl acetate (20 mL×3). The combined organic layers were washed with brine (30 mL), dried over anhydrous sodium sulfate, filtered and concentrated under reduced pressure to a residue. The concentrated residue was then purified by column chromatography (SiO₂, petroleum ether:ethyl acetate 5:1 to 1:1) to give the appropriate R¹-cyanide 6e which were used in the next step without further purification.

Step 3: To a solution of the appropriate R¹-cyanide 6e (287 μmol, 1.00 eq), HCl (6.0 M, 1.00 eq.) in MeOH (10 mL) was added palladium on activated carbon (296 μmol, 10/6 Pd) under nitrogen. The suspension was degassed under vacuum and purged with hydrogen several times. The mixture was then vigorously stirred under a hydrogen atmosphere (50 psi) at 40° C. for 2 hours before being filtered and concentrated under reduced pressure. The residue was purified by prep-HPLC (Waters Xbridge 150×25 mm×5 μm; mobile phase: [water (0.05% ammonia hydroxide v/v)-ACN]; B %: 12%-42%, 10 min) to return the desired compounds shown in Table 2.

Following the teachings of the General Reaction Schemes, the general reaction method 2 and the Intermediates disclosed herein, the Examples 2-2 to 2-5 are prepared as shown in Table 2.

TABLE 2

| Example | Structure | Compound Name and Characterization |
|---|---|---|
| 2-2 | 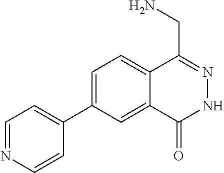 [SMILES: NCc1n[nH]c(=O)c2cc(-c3ccncc3)ccc12] | 4-(aminomethyl)-7-(pyridin-4-yl)phthalazin-1(2H)-one LCMS [M + 1]⁺ = 253.2; ¹H NMR (400 MHz, MeOD) δ = 8.73 (d, J = 2.0 Hz, 1H), 8.71-8.68 (m, 2H), 8.34 (dd, J = 2.0, 8.4 Hz, 1H), 8.15 (d, J = 8.6 Hz, 1H), 7.89-7.86 (m, 2H), 4.21 (s, 2H). |
| 2-3 | 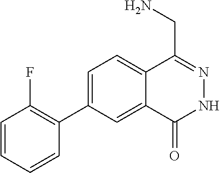 [SMILES: NCc1n[nH]c(=O)c2cc(-c3ccccc3F)ccc12] | 4-(aminomethyl)-7-(2-fluorophenyl)phthalazin-1(2H)- one LCMS [M + 1]⁺ = 270.2; ¹H NMR (400 MHz, MeOD) δ = 8.55 (s, 1H), 8.19-8.14 (m, 1H), 8.11-8.06 (m, 1H), 7.63 (dt, J = 1.6, 8.0 Hz, 1H), 7.52-7.45 (m, 1H), 7.35 (dt, J = 1.2, 7.6 Hz, 1H), 7.28 (ddd, J = 1.2, 8.4, 11.2 Hz, 1H), 4.25 (s, 2H). |
| 2-4 | 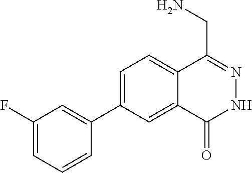 [SMILES: NCc1n[nH]c(=O)c2cc(-c3cccc(F)c3)ccc12] | 4-(aminomethyl)-7-(3-fluorophenyl)phthalazin-1(2H)- one LCMS [M + 1]⁺ = 270.2; ¹H NMR (400 MHz, MeOD) δ = 8.62 (d, J = 2.0 Hz, 1H), 8.25 (dd, J = 2.0, 8.6 Hz, 1H), 8.10 (d, J = 8.6 Hz, 1H), 7.65-7.61 (m, 1H), 7.60-7.53 (m, 2H), 7.26-7.18 (m, 1H), 4.22 (s, 2H). |
| 2-5 | 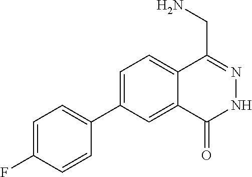 [SMILES: NCc1n[nH]c(=O)c2cc(-c3ccc(F)cc3)ccc12] | 4-(aminomethyl)-7-(4-fluorophenyl)phthalazin-1(2H)- one LCMS [M + 1]⁺ = 270.2; ¹H NMR (400 MHz, MeOD) δ = 8.58 (d, J = 2.0 Hz, 1H), 8.22 (dd, J = 2.0, 8.6 Hz, 1H), 8.08 (d, J = 8.4 Hz, 1H), 7.86-7.78 (m, 2H), 7.33-7.25 (m, 2H), 4.24 (s, 2H). |

### General Coupling Methods (CM) and Purification Methods (PM) for the Preparation of Examples 3-1 to 3-61

### CM 3A:

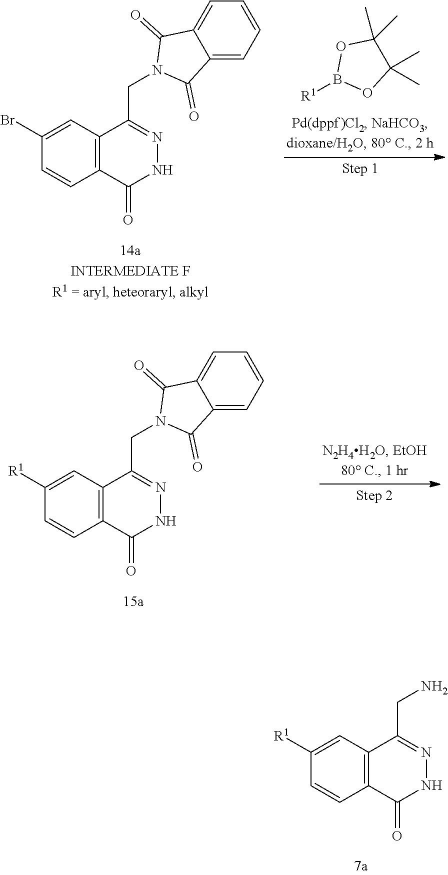

Step 1: A mixture of the appropriate aryl/heteroaryl/alkyl-boronic ester (390 μmol, 1.5 eq.), intermediate F (260 μmol, 1.00 eq.), Pd(dppf)Cl₂ (26 μmol, 0.10 eq.), sodium bicarbonate (43 mg, 521 μmol, 20.3 μL, 2.00 eq.) in dioxane (1.0 mL) and water (0.20 mL) was purged with nitrogen 3 times. The mixture was then stirred at 80° C. for 2 hours. After such time the mixture was filtered, washed with a dichloromethane:methyl alcohol 10:1 mixture and the filtrate concentrated under reduced pressure. The concentrated residue was then triturated with methyl alcohol (3.0 mL) to give the corresponding Suzuki coupling product 15a as a black solid.

Step 2: To a solution of corresponding Suzuki coupling product 15a in ethyl alcohol (1.0 mL) was added hydrazine hydrate (242 μmol, 14 μL). The mixture was stirred at 80° C. for 1 hour, cooled and concentrated under reduced pressure. The concentrated residue was then purified by prep-HPLC according to one of the purification methods 3-1, 3-2, 3-3 or 3-4 described herein to furnish 7a.

### CM 3B:

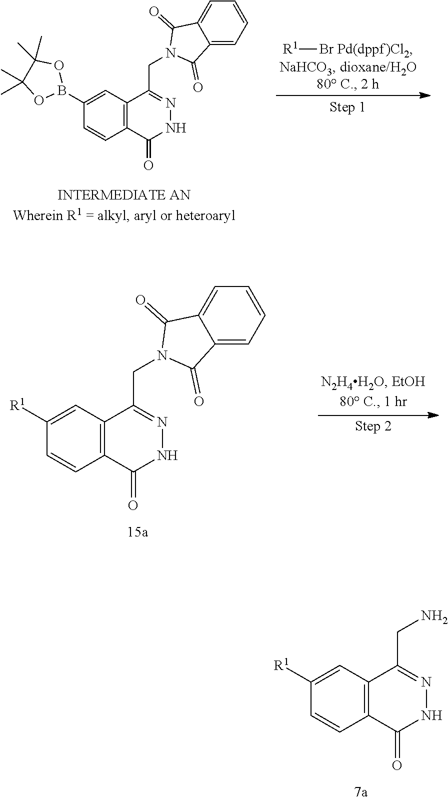

Step 1: Intermediate AN, the appropriate alkyl/aryl/heteroaryl bromide (464 μmol), sodium bicarbonate (464 μmol, 18.0 μL, 2.00 eq.) and Pd(dppf)Cl₂ (17 mg, 23 μmol, 0.10 eq.) in dioxane (2 mL) and water (0.40 mL) was purged with nitrogen 3 times. The mixture was then stirred at 80° C. for 1 hour. After such time the reaction mixture was diluted with water (2 mL), filtered under reduced pressure and the filter cake triturated with ethyl alcohol (3 mL) to give the corresponding R¹-Suzuki coupling product 15a as a black solid which was directly in the next step without further purification.

Step 2: To a solution of corresponding R¹-Suzuki coupling product 15a in ethyl alcohol (1.0 mL) was added hydrazine hydrate (242 μmol, 14 μL). The mixture was stirred at 80° C. for 1 hour. After such time the mixture was concentrated under reduced pressure and the residue purified by prep-HPLC according to one of the purification methods 3-1 to 3-4.

### CM 3C

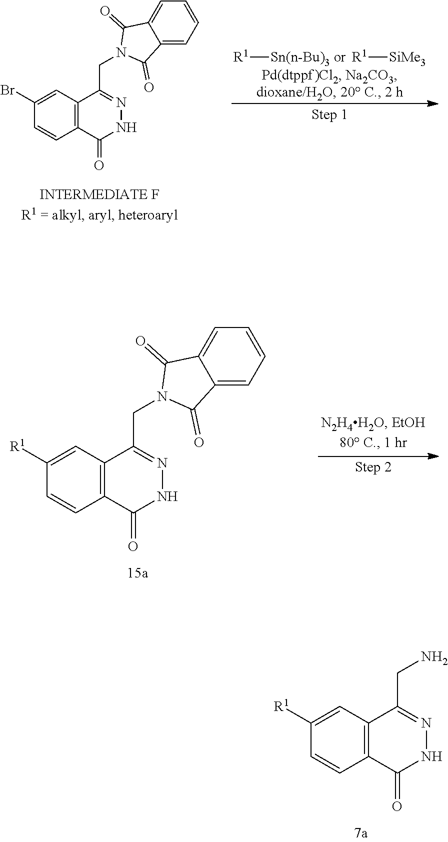

Step 1: A mixture of Intermediate F (312 μmol, 1.00 eq.), the appropriate alkyl/aryl/heteroaryl tributyltin or alkyl/aryl/heteroaryl trimethyl silicon reagent (625 μmol, 2.00 eq.), Pd(PPh₃)₄ (72 mg, 62 μmol, 0.20 eq.) in dioxane (3.0 mL) was purged with nitrogen 3 times. The mixture was then stirred at 100° C. for 1 hour. After such time the reaction mixture was diluted with a potassium fluoride solution (3.0 mL) to form a suspension. The suspension was filtered, and the filtrate extracted with ethyl acetate (8 mL×3). The combined organic layers were washed with brine (10 mL), dried over anhydrous sodium sulfate, filtered and concentrated under reduced pressure. The residue was purified by prep-TLC (SiO₂, dichloromethane:methyl alcohol 10:1) to give the corresponding C—C bond formation compound 15a as a black solid.

Step 2: To a solution of 15a in ethyl alcohol (1.0 mL) was added hydrazine hydrate (242 μmol, 14 μL). The mixture was stirred at 80° C. for 1 hour. After such time the mixture was concentrated under reduced pressure and the residue was purified by prep-HPLC according to one of the purification methods 3-1 to 3-4.

### Purification Methods (PM)

PM 3-1: column: Phenomenex Synergi C18 150×25×10 μm; mobile phase: [water (0.05% HCl)-ACN]; B %: 5%-25%, 11 min.

PM 3-2: column: Waters Xbridge 150×25 mm×5 μm; mobile phase: [water (0.05% ammonia hydroxide v/v)-ACN]; B %: 12%-42%, 10 min.

PM 3-3: column: Waters Xbridge 150×25 mm 5 μm; mobile phase: [water (10 mM NH₄HCO₃)-ACN]; B %: 2%-32%, 10 min.

PM 3-4: column: Phenomenex Luna C18 75×10 mm×3 μm; mobile phase: [water (0.05% HCl)-ACN]; B %: 0.1%-31%, 6.5 min.

Following the teachings of the General Reaction Schemes, the coupling methods 3A, 3B and 3C and using purification methods 3-1, 3-2, 3-3 and 3-4 and the Intermediates disclosed herein, the Examples 3-1 to 3-61 are prepared as shown in Table 3.

TABLE 3

| Example | Structure | CM | PM | Compound Name and Characterization |
|---|---|---|---|---|
| 3-1 | 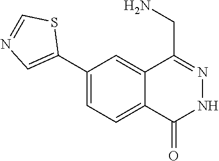 [SMILES: NCc1n[nH]c(=O)c2ccc(-c3cncs3)cc12] | 3C | 3-1 | 4-(aminomethyl)-6-(thiazol-5-yl)phthalazin- 1(2H)-one LCMS [M +1 ]⁺ = 259.2; ¹H NMR (400 MHz, DMSO-d6) δ = 12.97 (s, 1H), 9.27 (s, 1H), 8.71 (s, 1H), 8.59 (br s, 3H), 8.33 (d, J = 8.4 Hz, 1H), 8.27-8.24 (d, J = 1.6 Hz, 1H), 8.19 (dd, J = 1.6, 8.4 Hz, 1H), 4.59-4.50 (d, J = 5.6 Hz, 2H) |
| 3-2 | 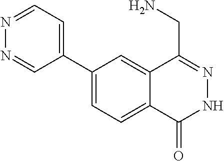 [SMILES: NCc1n[nH]c(=O)c2ccc(-c3ccnnc3)cc12] | 3B | 3-1 | 4-(aminomethyl)-6-(pyridazin-4-yl)phthal- azin-1(2H)-one LCMS [M + 1]⁺ = 253.26; ¹H NMR(400 MHz, DMSO-d6) δ = 13.06 (s, 1H), 9.97 (d, J = 1.2 Hz, 1H), 9.48 (d, J = 4.4 Hz, 1H), 8.68 (br s, 3H), 8.54 (s, 1H), 8.44 (m, 2H), 8.42 (dd, J = 2.4, 5.2 Hz, 1H), 4.59 (br d, J = 5.6 Hz, 2H) |
| 3-3 | 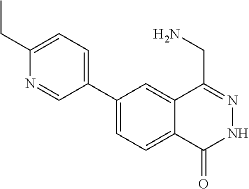 [SMILES: CCc1ccc(-c2ccc3c(=O)[nH]nc(CN)c3c2)cn1] | 3A | 3-1 | 4-(aminomethyl)-6-(6-ethylpyridin-3- yl)phthalazin-1(2H)-one LCMS [M + 1]⁺ = 281.3; ¹H NMR (400 MHz, DMSO-d6) δ = 13.01 (s, 1H), 9.33 (d, J = 2.0 Hz, 1H), 8.85 (br d, J = 7.6 Hz, 1H), 8.66 (br s, 3H), 8.43 (d, J = 1.2 Hz, 1H), 8.42-8.39 (m, 1H), 8.37- 8.32 (m, 1H), 7.95 (br d, J = 8.4 Hz, 1H), 4.70-4.48 (m, 2H), 3.07 (q, J = 7.6 Hz, 2H), 1.34 (t, J = 7.6 Hz, 3H) |
| 3-4 | 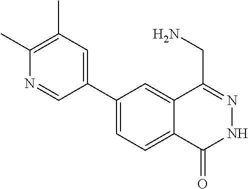 [SMILES: Cc1cc(-c2ccc3c(=O)[nH]nc(CN)c3c2)cnc1C] | 3A | 3-3 | 4-(aminomethyl)-6-(5,6-dimethylpyridin-3- yl)phthalazin-1(2H)-one LCMS [M + 1]⁺ = 281.3; ¹H NMR (400 MHz, DMSO-d6) δ = 8.72 (s, 1H), 8.35 (d, J = 8.4 Hz, 1H), 8.28 (s, 1H), 8.11 (d, J = 8.8 Hz, 1H), 7.96 (s, 1H), 4.12 (s, 2H), 2.52 (s, 3H), 2.39 (s, 3H) |
| 3-5 | 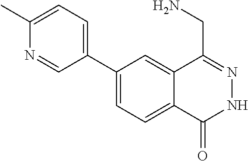 [SMILES: Cc1ccc(-c2ccc3c(=O)[nH]nc(CN)c3c2)cn1] | 3A | 3-3 | 4-(aminomethyl)-6-(6-methylpyridin-3- yl)phthalazin-1(2H)-one LCMS [M + 1]⁺ = 267.3; ¹H NMR (400 MHz, DMSO-d6) δ = 8.91 (d, J = 2.0 Hz, 1H), 8.36 (d, J = 8.0 Hz, 1H), 8.30 (d, J = 1.6 Hz, 1H), 8.11 (dd, J = 2.0, 8.0 Hz, 2H), 7.40 (d, J = 8.0 Hz, 1H), 4.12 (s, 2H), 2.57 (s, 3H) |
| 3-6 | 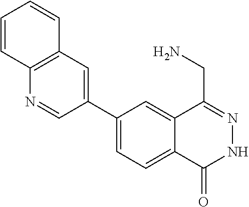 [SMILES: NCc1n[nH]c(=O)c2ccc(-c3cnc4ccccc4c3)cc12] | 3A | 3-2 | 4-(aminomethyl)-6-(quinolin-3-yl)phthalazin- 1(2H)-one LCMS [M + 1]⁺ = 303.2; ¹H NMR (400 MHz, DMSO-d6) δ = 12.55 (br s, 1H), 9.43 (d, J = 2.4 Hz, 1H), 8.90 (d, J = 2.4 Hz, 1H), 8.52 (d, J = 1.2 Hz, 1H), 8.43-8.38 (m, 1H), 8.38-8.33 (m, 1H), 8.12 (dd, J = 4.8, 7.6 Hz, 2H), 7.84 (dt, J = 1.2, 7.6 Hz, 1H), 7.75- 7.67 (m, 1H), 4.14 (s, 2H) |
| 3-7 | 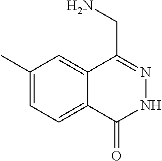 | 3A | 3-1 | 4-(aminomethyl)-6-methylphthalazin-1(2H)- one LCMS [M+1] + = 189.21; ¹H NMR (400 MHz, DMSO-d6) δ = 12.39 (brs, 1H), 8.14 (d, J = 8.0 Hz, 1H), 7.88 (s, 1H), 7.66 (dd, J = 0.8, 8.0 Hz, 1H), 3.98 (s, 2H), 2.52 (br s, 3H) |
| 3-8 | 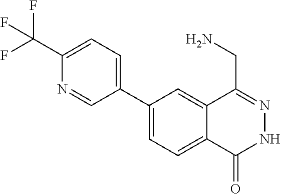 [SMILES: NCc1n[nH]c(=O)c2ccc(-c3ccc(C(F)(F)F)nc3)cc12] | 3A | 3-1 | 4-(aminomethyl)-6-(6-(trifluoromethyl)- pyridin-3-yl)phthalazin-1(2H)-one LCMS [M + 1]⁺ = 320.9; ¹H NMR (400 MHz, DMSO-d6) δ= 13.04 (s, 1H), 9.35 (d, J = 2.0 Hz, 1H), 8.64 (dd, J = 2.0, 8.4 Hz, 1H), 8.50 (br s, 3H), 8.46-8.41 (m, 1H), 8.40 (s, 1H), 8.38-8.34 (m, 1H), 8.13 (d, J = 8.0 Hz, 1H), 4.60 (br s, 2H) |
| 3-9 | 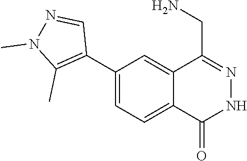 [SMILES: Cc1c(-c2ccc3c(=O)[nH]nc(CN)c3c2)cnn1C] | 3A | 3-1 | 4-(aminomethyl)-6-(1,5-dimethyl-1H-pyrazol- 4-yl)phthalazin-1(2H)-one LCMS [M + 1]⁺ = 270.1; ¹H NMR (400 MHz, DMSO-d6) δ = 12.9 (s, 1H), 8.48 (br s, 3H), 8.29 (d, J = 8.4 Hz, 1H), 7.97 (dd, J = 1.6, 8.4 Hz, 1H), 7.90 (s, 2H), 4.52 (br d, J = 5.6 Hz, 2H), 3.83 (s, 3H), 2.52 (br s, 3H) |
| 3-10 | 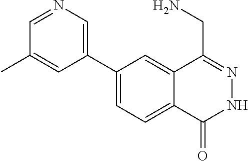 [SMILES: Cc1cncc(-c2ccc3c(=O)[nH]nc(CN)c3c2)c1] | 3A | 3-1 | 4-(aminomethyl)-6-(5-methylpyridin-3- yl)phthalazin-1(2H)-one LCMS [M + 1]⁺ = 267.0; ¹H NMR (400 MHz, DMSO-d6) δ = 13.0 (s, 1H), 9.18 (s, 1H), 8.71 (s, 1H), 8.59 (br s, 1H), 8.56 (br s, 3H), 8.45-8.39 (m, 1H), 8.38 (s, 1H), 8.36-8.32 (m, 1H), 4.59 (br d, J = 5.6 Hz, 2H), 2.54-2.52 (s, 3H) |
| 3-11 | 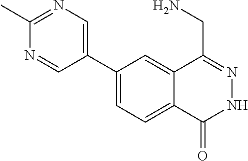 [SMILES: Cc1ncc(-c2ccc3c(=O)[nH]nc(CN)c3c2)cn1] | 3A | 3-1 | 4-(aminomethyl)-6-(2-methylpyrimidin-5- yl)phthalazin-1 (2H)-one LCMS [M + 1]⁺ = 268.3; ¹H NMR (400 MHz, DMSO-d6) δ = 12.99 (s, 1H), 9.34 (s, 2H), 8.71 (br s, 3H), 8.43-8.30 (m, 3H), 4.56 (br d, J = 5.6 Hz, 2H), 2.73 (s, 3H) |
| 3-12 | 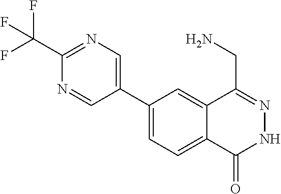 [SMILES: NCc1n[nH]c(=O)c2ccc(-c3cnc(C(F)(F)F)nc3)cc12] | 3A | 3-2 | 4-(aminomethyl)-6-(2-(trifluoromethyl)- pyrimidin-5-yl)phthalazin-1(2H)-one LCMS [M + 1]⁺ = 322.2; ¹H NMR (400 MHz, DMSO-d6) δ = 9.53 (s, 2H), 8.51 (s, 1H), 8.43 (d, J = 8.4 Hz, 1H), 8.29 (dd, J = 1.6, 8.4 Hz, 1H), 4.13 (s, 2H) |
| 3-13 | 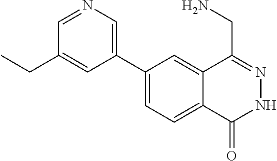 [SMILES: CCc1cncc(-c2ccc3c(=O)[nH]nc(CN)c3c2)c1] | 3A | 3-2 | 4-(aminomethyl)-6-(5-ethylpyridin-3-yl)- phthalazin-1(2H)-one LCMS [M + 1]⁺ = 281.3; ¹H NMR (400 MHz, DMSO-d6) δ = 8.86 (d, J = 2.0 Hz, 1H), 8.54 (d, J = 2.0 Hz, 1H), 8.36 (d, J = 8.4 Hz, 1H), 8.33 (d, J = 1.6 Hz, 1H), 8.15 (dd, J = 2.0, 8.4 Hz, 1H), 8.06 (s, 1H), 4.13 (s, 2H), 2.77 (q, J = 7.6 Hz, 2H), 1.31 (t, J = 7.6 Hz, 3H) |
| 3-14 | 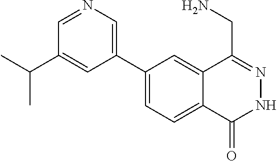 [SMILES: CC(C)c1cncc(-c2ccc3c(=O)[nH]nc(CN)c3c2)c1] | 3B | 3-1 | 4-(aminomethyl)-6-(5-isopropylpyridin-3- yl)phthalazin-1(2H)-one LCMS [M + 1]⁺ = 295.1; ¹H NMR (400 MHz, DMSO-d6) δ = 13.03 (s, 1H), 9.28 (br s, 1H), 8.82 (s, 1H), 8.79-8.74 (m, 1H), 8.65 (br s, 3H), 8.44-8.37 (m, 3H), 4.60 (q, J = 5.6 Hz, 2H), 3.25-3.17 (m, 1H), 1.37 (d, J = 7.2 Hz, 6H) |
| 3-15 | 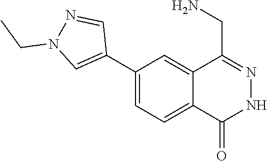 [SMILES: CCn1cc(-c2ccc3c(=O)[nH]nc(CN)c3c2)cn1] | 3A | 3-1 | 4-(aminomethyl)-6-(1-ethyl-1H-pyrazol-4- yl)phthalazin-1(2H)-one LCMS [M + 1]⁺ = 270.0; ¹H NMR (400 MHz, DMSO-d6) δ = 12.81 (s, 1H), 8.56 (m, 4H), 8.24 (d, J = 8.8 Hz, 1H), 8.20 (s, 1H), 8.14-8.09 (m, 2H), 4.48 (br d, J = 5.6 Hz, 2H), 4.19 (d, J = 7.2 Hz, 2H), 1.43 (t, J = 7.2 Hz, 3H). |
| 3-16 | 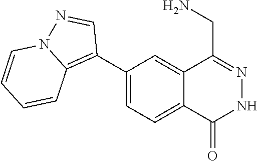 [SMILES: NCc1n[nH]c(=O)c2ccc(-c3cnn4ccccc34)cc12] | 3A | 3-1 | 4-(aminomethyl)-6-(pyrazolo[1,5-a]pyridin- 3-yl)phthalazin-1(2H)-one LCMS [M + 1]⁺ = 292.0; ¹H NMR (400 MHz, DMSO-d6) δ = 12.88 (s, 1H), 8.84 (d, J = 6.8 Hz, 1H), 8.74 (s, 1H), 8.59 (br s, 3H), 8.37-8.31 (m, 1H), 8.30-8.24 (m, 2H), 8.16 (d, J = 1.2 Hz, 1H), 7.48 (ddd, J = 1.2, 6.8, 9.2 Hz, 1H), 7.08 (dt, J = 1.2, 6.8 Hz, 1H), 4.61 (br s, 2H) |
| 3-17 | 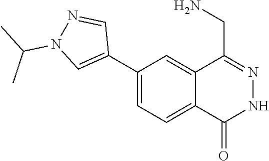 [SMILES: CC(C)n1cc(-c2ccc3c(=O)[nH]nc(CN)c3c2)cn1] | 3A | 3-2 | 4-(aminomethyl)-6-(1-isopropyl-1H-pyrazol- 4-yl)phthalazin-1(2H)-one LCMS [M + 1]⁺ = 284.3; ¹H NMR (400 MHz, DMSO-d6) δ = 8.54 (s, 1H), 8.20 (d, J = 8.4 Hz, 1H), 8.15 (d, J = 1.2 Hz, 1H), 8.13 (s, 1H), 8.06 (dd, J = 1.6, 8.4 Hz, 1H), 4.59-4.47 (m, 1H), 4.05 (s, 2H), 1.48 (d, J = 6.8 Hz, 6H) |
| 3-18 | 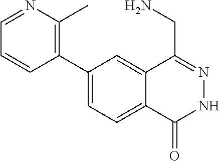 [SMILES: Cc1ncccc1-c1ccc2c(=O)[nH]nc(CN)c2c1] | 3A | 3-2 | 4-(aminomethyl)-6-(2-methylpyridin-3- yl)phthalazin-1(2H)-one LCMS [M + 1]⁺ = 267.3; ¹H NMR (400 MHz, DMSO-d6) δ = 12.53 (br s, 1H), 8.54 (dd, J = 1.6, 4.8 Hz, 1H), 8.32 (d, J = 8.4 Hz, 1H), 8.11 (d, J = 1.6 Hz, 1H), 7.88 (dd, J = 1.6, 8.0 Hz, 1H), 7.76 (dd, J = 1.6, 7.6 Hz, 1H), 7.38 (dd, J = 4.8, 7.6 Hz, 1H), 4.02 (s, 2H), 2.47 (s, 3H) |
| 3-19 | 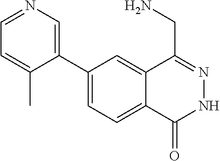 [SMILES: Cc1ccncc1-c1ccc2c(=O)[nH]nc(CN)c2c1] | 3A | 3-3 | 4-(aminomethyl)-6-(4-methylpyridin-3-yl)- phthalazin-1(2H)-one LCMS [M + 1]⁺ = 267.3; ¹H NMR (400 MHz, DMSO-d6) δ = 8.52-8.49 (m, 2H), 8.36 (d, J = 8.0 Hz, 1H), 8.08 (d, J = 1.2 Hz, 1H), 7.84 (dd, J = 1.6, 8.0 Hz, 1H), 7.38 (d, J = 5.2 Hz, 1H), 4.07 (s, 2H), 2.32 (s, 3H) |
| 3-20 | 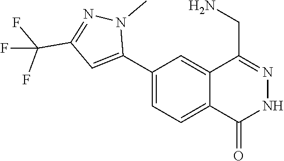 [SMILES: Cn1nc(C(F)(F)F)cc1-c1ccc2c(=O)[nH]nc(CN)c2c1] | 3A | 3-2 | 4-(aminomethyl)-6-(1-methyl-3-(trifluoro- methyl)-1H-pyrazol-5-yl)phthalazin-1(2H)- one LCMS [M + 1] ⁺ 323.9; ¹H NMR (400 MHz, DMSO-d6) δ = 12.71 (br s, 1H), 8.37 (d, J = 8.0 Hz, 1H), 8.26 (d, J = 1.2 Hz, 1H), 8.08 (dd, J = 1.6, 8.4 Hz, 1H), 7.18 (s, 1H), 4.17 (s, 2H), 4.02 (s, 3H), 2.52 (br s, 2H) |
| 3-21 | 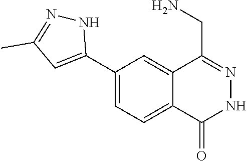 [SMILES: Cc1cc(-c2ccc3c(=O)[nH]nc(CN)c3c2)[nH]n1] | 3A | 3-1 | 4-(aminomethyl)-6-(5-methyl-1H-pyrazol-3- yl)phthalazin-1(2H)-one LCMS [M + 1]⁺ = 256.0; ¹H NMR (400 MHz, DMSO-d6) δ = 12.86 (s, 1H), 8.55 (br s, 3H), 8.32-8.26 (m, 2H), 8.24 (s, 1H), 6.82 (d, J = 0.8 Hz, 1H), 4.60-4.43 (m, 2H), 2.37-2.22 (m, 3H) |
| 3-22 | 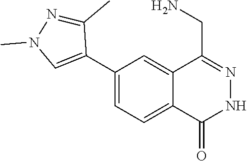 [SMILES: Cc1nn(C)cc1-c1ccc2c(=O)[nH]nc(CN)c2c1] | 3A | 3-2 | 4-(aminomethyl)-6-(1,3-dimethyl-1H- pyrazol-4-yl)phthalazin-1(2H)-one LCMS [M + 1]⁺ = 270.3; ¹H NMR (400 MHz, DMSO-d6) δ = 12.43 (br s, 1H), 8.23 (d, J = 8.4 Hz, 1H), 8.19 (s, 1H), 8.04 (d, J = 1.2 Hz, 1H), 7.91 (dd, J = 1.6, 8.4 Hz, 1H), 4.06 (s, 2H), 3.83 (s, 3H), 2.40 (s, 3H) |
| 3-23 | 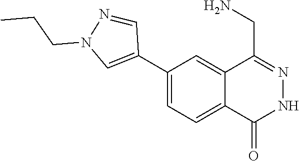 [SMILES: CCCn1cc(-c2ccc3c(=O)[nH]nc(CN)c3c2)cn1] | 3A | 3-2 | 4-(aminomethyl)-6-(1-propyl-1H-pyrazol-4- yl)phthalazin-1(2H)-one LCMS [M + 1]⁺ = 284.3; ¹H NMR (400 MHz, DMSO-d6) δ = 12.49-12.35 (s, 1H), 8.49 (s, 1H), 8.20 (d, J = 8.4 Hz, 1H), 8.15 (s, 2H), 8.05 (dd, J = 1.6, 8.4 Hz, 1H), 4.12 (t, J = 6.8 Hz, 2H), 4.08 (s, 2H), 1.89-1.81 (m, 2H), 0.87 (t, J = 7.2 Hz, 3H) |
| 3-24 | 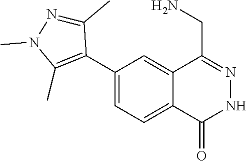 [SMILES: Cc1nn(C)c(C)c1-c1ccc2c(=O)[nH]nc(CN)c2c1] | 3A | 3-1 | 4-(aminomethyl)-6-(1,3,5-trimethyl-1H- pyrazol-4-yl)phthalazin-1(2H)-one LCMS [M + 1]⁺ = 284.0; ¹H NMR (400 MHz, DMSO-d6) δ = 12.9 (s, 1H), 8.63 (br s, 3H), 8.31 (d, J = 8.4 Hz, 1H), 7.81 (dd, J = 1.2, 8.4 Hz, 1H), 7.76 (s, 1H), 4.46 (br d, J = 5.6 Hz, 2H), 3.76 (s, 3H), 2.32 (s, 3H), 2.22 (s, 3H) |
| 3-25 | 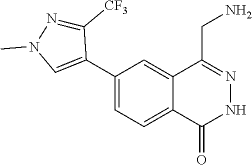 [SMILES: Cn1cc(-c2ccc3c(=O)[nH]nc(CN)c3c2)c(C(F)(F)F)n1] | 3A | 3-2 | 4-(aminomethyl)-6-(1-methyl-3-(trifluoro- methyl)-1H-pyrazol-4-yl)phthalazin-1(2H)- one LCMS [M + 1]⁺ = 324.2 ¹H NMR (400 MHz, DMSO-d6) δ = 12.52 (br s, 1H), 8.40 (s, 1H), 8.30 (d, J = 8.0 Hz, 1H), 8.07 (s, 1H), 7.83 (br d, J = 8.8 Hz, 1H), 4.00 (s, 5H) |
| 3-26 | 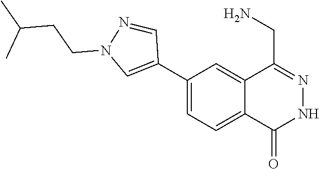 [SMILES: CC(C)CCn1cc(-c2ccc3c(=O)[nH]nc(CN)c3c2)cn1] | 3A | 3-1 | 4-(aminomethyl)-6-(1-isopentyl-1H-pyrazol- 4-yl)phthalazin-1(2H)-one LCMS [M + 1]⁺ = 312.1 ¹H NMR (400 MHz, DMSO-d6) δ = 12.84 (s, 1H), 8.59 (s, 4H), 8.26-8.20 (m, 2H), 8.14 (d, J = 1.6 Hz, 1H), 8.12 (s, 1H), 4.57-4.43 (m, 2H), 4.19 (t, J = 7.2 Hz, 2H), 1.81-1.69 (m, 2H), 1.52 (td, J = 6.8, 13.2 Hz, 1H), 0.93 (d, J = 6.4 Hz, 6H) |
| 3-27 | 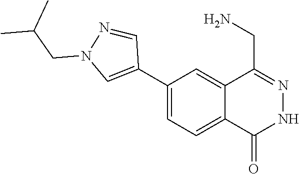 [SMILES: CC(C)Cn1cc(-c2ccc3c(=O)[nH]nc(CN)c3c2)cn1] | 3A | 3-1 | 4-(aminomethyl)-6-(1-isobutyl-1H-pyrazol- 4-yl)phthalazin-1(2H)-one LCMS [M + 1]⁺ = 296.0 ¹H NMR (400 MHz, DMSO-d6) δ = 12.9-12.8 (s, 1H), 8.49 (s, 1H), 8.44-8.36 (s, 3H), 8.25 (d, J = 8.4 Hz, 1H), 8.20 (s, 1H), 8.13 (dd, J = 1.6, 8.4 Hz, 1H), 8.08 (s, 1H), 4.51 (q, J = 5.6 Hz, 2H), 3.98 (d, J = 7.2 Hz, 2H), 2.22-2.14 (m, 1H), 0.89 (d, J = 6.8 Hz, 6H). |
| 3-28 | 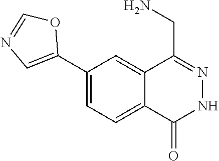 [SMILES: NCc1n[nH]c(=O)c2ccc(-c3cnco3)cc12] | 3A | 3-3 | 4-(aminomethyl)-6-(oxazol-5-yl)phthalazin- 1(2H)-one LCMS [M + 1]⁺ = 243.2; ¹H NMR (400 MHz, DMSO-d6) δ = 8.51 (s, 1H), 8.35 (br s, 2H), 8.15 (br d, J = 8.4 Hz, 1H), 7.96 (s, 1H), 4.09 (s, 2H). |
| 3-29 | 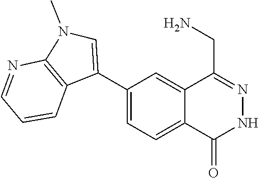 [SMILES: Cn1cc(-c2ccc3c(=O)[nH]nc(CN)c3c2)c2cccnc21] | 3A | 3-1 | 4-(aminomethyl)-6-(1-methyl-1H-pyrrolo- [2,3-b]pyridin-3-yl)phthalazin-1(2H)-one LCMS [M + 1]⁺ = 306.1 ¹H NMR (400 MHz, DMSO-d6) δ = 12.86 (s, 1H), 8.57 (dd, J = 1.6, 8.0 Hz, 1H), 8.51 (br s, 3H), 8.40 (dd, J = 1.2, 4.4 Hz, 1H), 8.37 (s, 1H), 8.35-8.31 (m, 1H), 8.30-8.26 (m, 1H), 8.14 (d, J = 1.2 Hz, 1H), 7.29 (dd, J = 4.8, 8.0 Hz, 1H), 4.61 (br d, J = 5.6 Hz, 2H), 3.94 (s, 3H) |
| 3-30 | 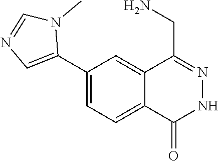 [SMILES: Cn1cncc1-c1ccc2c(=O)[nH]nc(CN)c2c1] | 3A | 3-1 | 4-(aminomethyl)-6-(1-methyl-1H-imidazol- 5-yl)phthalazin-1(2H)-one LCMS [M + 1]⁺ = 256.1 ¹H NMR (400 MHz, DMSO-d6) δ = 13.08 (s, 1H), 9.15 (s, 1H), 8.62-8.53 (br s, 3H), 8.43 (d, J = 8.4 Hz, 1H), 8.22 (d, J = 1.2 Hz, 1H), 8.14 (dd, J = 1.6, 8.4 Hz, 1H), 8.07 (s, 1H), 4.54-4.47 (m, 2H), 3.95 (s, 3H) |
| 3-31 | 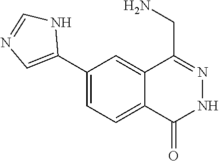 [SMILES: NCc1n[nH]c(=O)c2ccc(-c3cnc[nH]3)cc12] | 3A | 3-1 | 4-(aminomethyl)-6-(1H-imidazol-5-yl)- phthalazin-1(2H)-one LCMS [M + 1]⁺ = 242.0 ¹H NMR (400 MHz, DMSO-d6) δ = 12.99 (s, 1H), 9.11 (br s, 1H), 8.62 (s, 1H), 8.58 (br s, 3H), 8.46 (s, 1H), 8.38 (m, 2H), 4.53 (br s, 2H) |
| 3-32 | 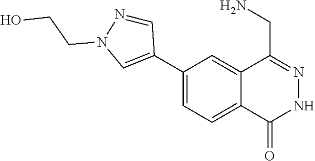 [SMILES: NCc1n[nH]c(=O)c2ccc(-c3cnn(CCO)c3)cc12] | 3A | 3-1 | 4-(aminomethyl)-6-(1-(2-hydroxyethyl)-1H- pyrazol-4-yl)phthalazin-1(2H)-one LCMS [M + 1]⁺ = 286.1 ¹H NMR (400 MHz, DMSO-d6) δ = 12.84 (s, 1H), 8.53-8.49 (s, 1H), 8.46 (br s, 3H), 8.27-8.19 (m, 2H), 8.16- 8.11 (m, 1H), 8.09 (s, 1H), 4.55-4.47 (m, 2H), 4.20 (br t, J = 5.6 Hz, 2H), 3.79 (br t, J = 5.6 Hz, 2H) |
| 3-33 | 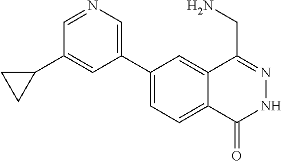 [SMILES: NCc1n[nH]c(=O)c2ccc(-c3cncc(C4CC4)c3)cc12] | 3C | 3-1 | 4-(aminomethyl)-6-(5-cyclopropylpyridin-3- yl)phthalazin-1(2H)-one LCMS [M + 1]⁺ = 293.1 ¹H NMR (400 MHz, DMSO-d6) δ = 13.04 (s, 1H), 9.32 (d, J = 2.0 Hz, 1H), 8.86- 8.69 (m, 4H), 8.65 (s, 1H), 8.48 (s, 1H), 8.40 (s, 2H), 4.59 (br d, J = 5.6 Hz, 2H), 2.36- 2.25 (m, 1H), 1.29-1.03 (m, 4H) |
| 3-34 | 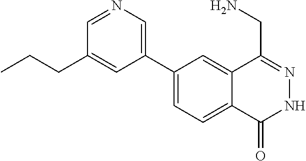 [SMILES: CCCc1cncc(-c2ccc3c(=O)[nH]nc(CN)c3c2)c1] | 3A | 3-1 | 4-(aminomethyl)-6-(5-propylpyridin-3-yl)- phthalazin-1(2H)-one ¹H NMR (400 MHz, MeOD) δ = 8.82 (s, 1H), 8.51-8.47 (m, 2H), 8.24 (s, 1H), 8.20-8.13 (m, 2H), 4.27 (s, 2H), 2.78 (t, J = 7.6 Hz, 2H), 1.02 (t, J = 7.6 Hz, 3H) |
| 3-35 | 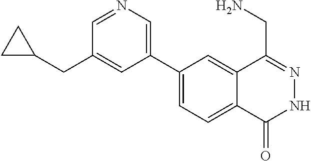 [SMILES: NCc1n[nH]c(=O)c2ccc(-c3cncc(CC4CC4)c3)cc12] | 3A | 3-2 | 4-(aminomethyl)-6-(5-(cyclopropylmethyl)- pyridin-3-yl)phthalazin-1(2H)-one LCMS [M + 1]⁺ 307.2 ¹H NMR (400 MHz, DMSO- d6) δ = 12.76-12.38 (m, 1H), 8.93 (d, J = 2.0 Hz, 1H), 8.59 (d, J = 1.6 Hz, 1H), 8.39-8.32 (m, 2H), 8.23-8.19 (m, 1H), 8.17 (t, J = 2.0 Hz, 1H), 4.17 (s, 2H), 2.64 (d, J = 6.8 Hz, 2H), 1.14-1.04 (m, 1H), 0.55-0.48 (m, 2H), 0.33-0.26 (m, 2H) |
| 3-36 | 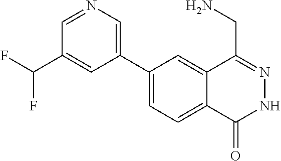 [SMILES: NCc1n[nH]c(=O)c2ccc(-c3cncc(C(F)F)c3)cc12] | 3B | 3-2 | 4-(aminomethyl)-6-(5-difluoromethyl)- pyridin-3-yl)phthalazin-1(2H)-one LCMS [M + 1]⁺ = 303.2 ¹H NMR (400 MHz, DMSO-d6) δ = 9.26-9.18 (m, 1H), 8.87 (d, J = 1.6 Hz, 1H), 8.44-8.36 (m, 3H), 8.22- 8.18 (m, 1H), 7.36-7.07 (m, 1H), 4.14 (s, 2H) |
| 3-37 | 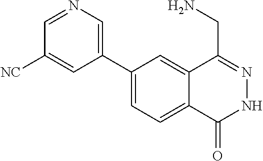 [SMILES: N#Cc1cncc(-c2ccc3c(=O)[nH]nc(CN)c3c2)c1] | 3B | 3-3 | 5-(4-(aminomethyl)-1-oxo-1,2-dihydro- phthalazin-6-yl)nicotinonitrile LCMS [M + 1]⁺ = 278.0 ¹H NMR (400 MHz, DMSO-d6) δ = 13.02 (s, 1H), 9.46 (d, J = 2.4 Hz, 1H), 9.14 (d, J = 2.0 Hz, 1H), 8.97 (t, J = 2.0 Hz, 1H), 8.62 (br s, 3H), 8.42-8.39 (m, 2H), 8.38-8.34 (m, 1H), 4.57 (q, J = 5.6 Hz, 2H) |
| 3-39 | 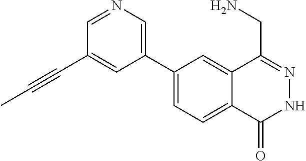 [SMILES: CC#Cc1cncc(-c2ccc3c(=O)[nH]nc(CN)c3c2)c1] | 3A | 3-1 | 4-(aminomethyl)-6-(5-(prop-1-yn-1-yl)- pyridin-3-yl)phthalazin-1(2H)-one LCMS [M + 1]⁺ = 291.0 ¹H NMR (400 MHz, DMSO-d6) δ = 12.55 (s, 1H), 9.02 (d, J = 2.4 Hz, 1H), 8.67 (d, J = 1.6 Hz, 1H), 8.38 (d, J = 1.2 Hz, 1H), 8.36-8.32 (m, 2H), 8.22 (dd, J = 1.6, 8.4 Hz, 1H), 4.12 (s, 2H), 2.13 (s, 3H) |
| 3-40 | 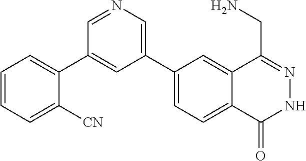 [SMILES: N#Cc1ccccc1-c1cncc(-c2ccc3c(=O)[nH]nc(CN)c3c2)c1] | 3A | 4-3 | 2-(5-(4-(aminomethyl)-1-oxo-1,2-dihydro- phthalazin-6-yl)pyridin-3-yl)benzonitrile LCMS [M + 1]⁺ = 354.0 ¹H NMR (400 MHz, DMSO-d6) δ = 13.02 (s, 1H), 9.35 (d, J = 2.0 Hz, 1H), 8.98 (d, J = 2.0 Hz, 1H), 8.74 (t, J = 2.0 Hz, 1H), 8.63 (br s, 3H), 8.46-8.40 (m, 3H), 8.08 (d, J = 7.6 Hz, 1H), 7.93-7.87 (m, 2H), 7.75-7.68 (m, 1H), 4.63-4.53 (m, 2H) |
| 3-41 | 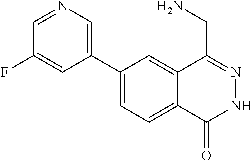 [SMILES: NCc1n[nH]c(=O)c2ccc(-c3cncc(F)c3)cc12] | 3A | 3-1 | 4-(aminomethyl)-6-(5-fluoropyridin-3-yl)- phthalazin-1(2H)-one LCMS [M + 1]⁺ = 271.1 ¹H NMR (400 MHz, DMSO-d6) δ = 13.0 (s, 1H), 9.08 (t, J = 1.6 Hz, 1H), 8.73 (d, J = 2.4 Hz, 1H), 8.51 (br s, 3H), 8.42 (m, 2H), 8.37-8.34 (m, 2H), 4.63-4.57 (m, 2H) |
| 3-42 | 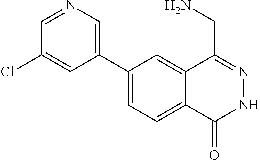 [SMILES: NCc1n[nH]c(=O)c2ccc(-c3cncc(Cl)c3)cc12] | 3A | 3-1 | 4-(aminomethyl)-6-(5-chloropyridin-3-yl)- phthalazin-1(2H)-oneLCMS [M + 1]⁺ 287.0 ¹H NMR (400 MHz, DMSO-d6) δ = 12.6 (br s, 1H), 9.06 (d, J = 2.0 Hz, 1H), 8.73 (d, J = 2.4 Hz, 1H), 8.51 (t, J = 2.0 Hz, 1H), 8.40 (d, J = 1.6 Hz, 1H), 8.34 (d, J = 8.4 Hz, 1H), 8.24 (dd, J = 1.6, 8.4 Hz, 1H), 4.10 (s, 2H), 2.31-1.93 (br s, 2H) |
| 3-43 | 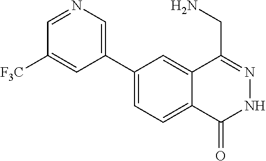 [SMILES: NCc1n[nH]c(=O)c2ccc(-c3cncc(C(F)(F)F)c3)cc12] | 3A | 4-3 | 4-(aminomethyl)-6-(5-(trifluoromethyl)- pyridin-3-yl)phthalazin-1(2H)-one LCMS [M + 1]⁺ = 321.0 ¹H NMR (400 MHz, DMSO-d6) δ = 13.03 (s, 1H), 9.47 (d, J = 2.4 Hz, 1H), 9.12-9.10 (s, 1H), 8.79 (s, 1H), 8.50 (br s, 3H), 8.42-8.39 (m, 3H), 4.64-4.58 (m, 2H) |
| 3-44 | 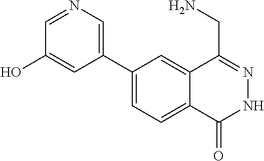 [SMILES: NCc1n[nH]c(=O)c2ccc(-c3cncc(O)c3)cc12] | 3A | 3-1 | 4-(aminomethyl)-6-(5-hydroxypyridin-3- yl)phthalazin-1(2H)-one LCMS [M + 1]⁺ = 269.1 ¹H NMR (400 MHz, DMSO-d6) δ = 13.02 (s, 1H), 11.61 (s, 1H), 8.91 (d, J = 1.2 Hz, 1H), 8.61 (br s, 3H), 8.47 (d, J = 2.4 Hz, 1H), 8.42-8.36 (m, 2H), 8.29 (dd, J = 1.6, 8.0 Hz, 1H), 8.24 (br s, 1H), 4.58 (br d, J = 5.6 Hz, 2H) |
| 3-45 | 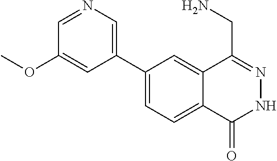 [SMILES: COc1cncc(-c2ccc3c(=O)[nH]nc(CN)c3c2)c1] | 3A | 3-1 | 4-(aminomethyl)-6-(5-methoxypyridin-3-yl)- phthalazin-1(2H)-one LCMS [M + 1]⁺ = 282.9 ¹H NMR (400 MHz, DMSO-d6) δ = 13.01 (s, 1H), 8.95 (d, J = 1.6 Hz, 1H), 8.67 (br s, 3H), 8.57 (d, J = 2.8 Hz, 1H), 8.42-8.34 (m, 3H), 8.28 (s, 1H), 4.69-4.44 (m, 2H), 4.03 (s, 3H) |
| 3-46 | 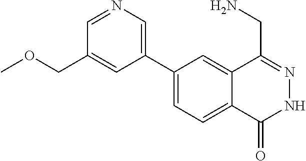 [SMILES: COCc1cncc(-c2ccc3c(=O)[nH]nc(CN)c3c2)c1] | 3A | 3-1 | 4-(aminomethyl)-6-(5-(methoxymethyl)- pyridin-3-yl)phthalazin-1(2H)-one LCMS [M + 1]⁺ = 297.2 ¹H NMR (400 MHz, DMSO-d6) δ = 12.54 (br s, 1H), 9.02 (d, J = 2.0 Hz, 1H), 8.63 (d, J = 2.0 Hz, 1H), 8.38- 8.34 (m, 2H), 8.23-8.18 (m, 2H), 4.57 (s, 2H), 4.11 (s, 2H), 3.37 (s, 3H), 2.30-1.93 (m, 2H) |
| 3-47 | 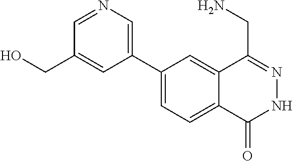 [SMILES: NCc1n[nH]c(=O)c2ccc(-c3cncc(CO)c3)cc12] | 3A | 3-2 | 4-(aminomethyl)-6-(5-(hydroxymethyl)- pyridin-3-yl)phthalazin-1(2H)-one LCMS [M + 1]⁺ = 283.2 ¹H NMR (400 MHz, DMSO-d6) δ = 12.48-12.06 (m, 1H), 8.92 (d, J = 2.0 Hz, 1H), 8.63 (d, J = 2.0 Hz, 1H), 8.38 (d, J = 8.4 Hz, 1H), 8.33 (d, J = 1.2 Hz, 1H), 8.16-8.12 (m, 2H), 5.15 (br s, 1H), 4.68 (s, 2H), 4.13 (s, 2H) |
| 3-48 | 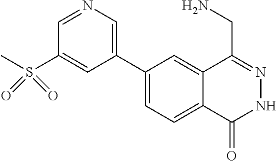 [SMILES: CS(=O)(=O)c1cncc(-c2ccc3c(=O)[nH]nc(CN)c3c2)c1] | 3A | 3-2 | 4-(aminomethyl)-6-(5-(methylsulfonyl)- pyridin-3-yl)phthalazin-1(2H)-one LCMS [M + 1]⁺ = 331.0 ¹H NMR (400 MHz, DMSO-d6) δ = 13.03 (s, 1H), 9.49 (d, J = 2.0 Hz, 1H), 9.17 (d, J = 2.0 Hz, 1H), 8.84 (t, J = 2.0 Hz, 1H), 8.61 (br s, 3H), 8.46-8.37 (m, 3H), 4.60 (s, 2H), 3.46 (s, 3H) |
| 3-49 | 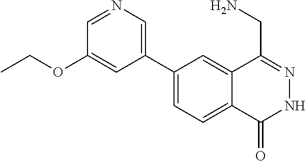 [SMILES: CCOc1cncc(-c2ccc3c(=O)[nH]nc(CN)c3c2)c1] | 3A | 3-1 | 4-(aminomethyl)-6-(5-ethoxypyridin-3-yl)- phthalazin-1(2H)-one LCMS [M + 1]⁺ = 297.0 ¹H NMR (400 MHz, DMSO-d6) δ = 13.02 (s, 1H), 8.97 (d, J = 1.6 Hz, 1H), 8.68 (br s, 3H), 8.58 (d, J = 2.8 Hz, 1H), 8.43-8.35 (m, 3H), 8.33 (s, 1H), 4.71-4.53 (m, 2H), 4.35 (q, J = 7.2 Hz, 2H), 1.43 (t, J = 7.2 Hz, 3H) |
| 3-50 | 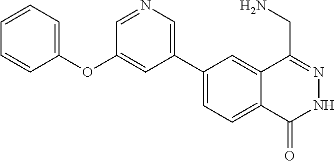 [SMILES: NCc1n[nH]c(=O)c2ccc(-c3cncc(Oc4ccccc4)c3)cc12] | 3A | 3-1 | 4-(aminomethyl)-6-(5-phenoxypyridin-3-yl)- phthalazin-1(2H)-one LCMS [M + 1]⁺ = 345.1 ¹H NMR (400 MHz, MeOH-d4) δ = 8.91 (s, 1H), 8.54 (d, J = 9.2 Hz, 1H), 8.46 (d, J = 2.4 Hz, 1H), 8.26-8.22 (m, 2H), 8.19 (t, J = 2.0 Hz, 1H), 7.52-7.47 (m, 2H), 7.31-7.26 (m, 1H), 7.23-7.19 (m, 2H), 4.69 (s, 2H) |
| 3-51 | 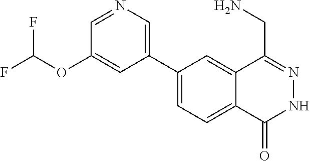 [SMILES: NCc1n[nH]c(=O)c2ccc(-c3cncc(OC(F)F)c3)cc12] | 3B | 3-1 | 4-(aminomethyl)-6-(5-(difluoromethoxy)- pyridin-3-yl)phthalazin-1(2H)-one LCMS [M + 1]⁺ = 318.9 ¹H NMR (400 MHz, DMSO-d6) δ = 13.00 (s, 1H), 9.08 (d, J = 2.0 Hz, 1H), 8.63 (br d, J = 2.8 Hz, 4H), 8.42- 8.36 (m, 2H), 8.35-8.30 (m, 2H), 7.77-7.31 (d, J = 73.2 Hz, 1H), 4.67-4.47 (m, 2H) |
| 3-52 | 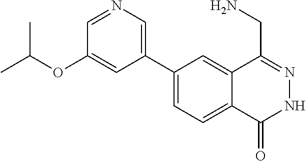 [SMILES: CC(C)Oc1cncc(-c2ccc3c(=O)[nH]nc(CN)c3c2)c1] | 3A | 3-2 | 4-(aminomethyl)-6-(5-isopropoxypyridin-3- yl)phthalazin-1(2H)-one LCMS [M + 1]⁺ = 311.2 ¹H NMR (400 MHz, DMSO-d6) δ = 12.54 (br s, 1H), 8.63 (d, J = 1.6 Hz, 1H), 8.38-8.27 (m, 3H), 8.19 (dd, J = 1.6, 8.4 Hz, 1H), 7.81 (t, J = 2.4 Hz, 1H), 4.90 (spt, J = 6.0 Hz, 1H), 4.10 (s, 2H), 1.33 (d, J = 6.0 Hz, 6H). |
| 3-53 | 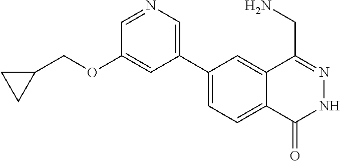 [SMILES: NCc1n[nH]c(=O)c2ccc(-c3cncc(OCC4CC4)c3)cc12] | 3A | 3-1 | 4-(aminomethyl)-6-(5-(cyclopropylmethoxy)- pyridin-3-yl)phthalazin-1(2H)-one LCMS [M + 1]⁺ = 323.1 ¹H NMR (400 MHz, DMSO-d6) δ = 13.02 (s, 1H), 9.00 (d, J = 1.6 Hz, 1H), 8.72 (br s, 3H), 8.59 (d, J = 2.4 Hz, 1H), 8.46-8.29 (m, 4H), 4.62-4.56 (m, 2H), 4.15 (d, J = 7.2 Hz, 2H), 1.40-1.16 (m, 1H), 0.76-0.56 (m, 2H), 0.45-0.29 (m, 2H) |
| 3-54 | 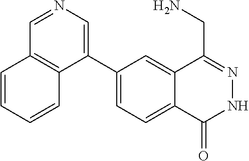 [SMILES: NCc1n[nH]c(=O)c2ccc(-c3cncc4ccccc34)cc12] | 3A | 3-1 | 4-(aminomethyl)-6-(isoquinolin-4-yl)- phthalazin-1(2H)-one LCMS [M + 1]⁺ = 303.0 ¹H NMR (400 MHz, DMSO-d6) δ = 13.08 (s, 1H), 9.85 (s, 1H), 8.82 (s, 1H), 8.66 (br s, 3H), 8.57 (d, J = 8.4 Hz, 1H), 8.49 (d, J = 8.4 Hz, 1H), 8.29 (d, J = 1.0 Hz, 1H), 8.19-7.93 (m, 4H), 4.51 (br d, J = 5.6 Hz, 2H) |
| 3-55 | 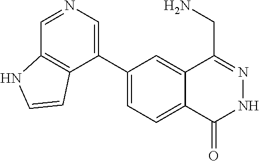 [SMILES: NCc1n[nH]c(=O)c2ccc(-c3cncc4[nH]ccc34)cc12] | 3A | 3-1 | 4-(aminomethyl)-6-(1H-pyrrolo[2,3-c]- pyridin-4-yl)phthalazin-1(2H)-one LCMS [M + 1]⁺ = 292.0; ¹H NMR (400 MHz, DMSO-d6) δ = 13.46 (br s, 1H), 13.05 (s, 1H), 9.27 (s, 1H), 8.77 (s, 1H), 8.67 (br s, 3H), 8.53-8.42 (m, 2H), 8.40-8.31 (m, 2H), 7.21 (br s, 1H), 4.60 (br d, J = 4.4 Hz, 2H) |
| 3-56 | 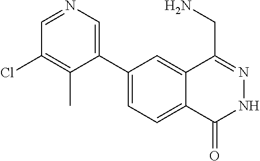 [SMILES: Cc1c(Cl)cncc1-c1ccc2c(=O)[nH]nc(CN)c2c1] | 3A | 3-1 | 4-(aminomethyl)-6-(5-chloro-4-methyl- pyridin-3-yl)phthalazin-1(2H)-one LCMS [M + 1]⁺ = 301.0; ¹H NMR (400 MHz, DMSO-d6) δ = 13.04-13.00 (s, 1H), 8.71 (s, 1H), 8.59-8.44 (m, 4H), 8.39 (d, J = 8.4 Hz, 1H), 8.07 (d, J = 1.2 Hz, 1H), 7.99 (dd, J = 1.6, 8.4 Hz, 1H), 4.54-4.45 (m, 2H), 2.33 (s, 3H) |
| 3-57 | 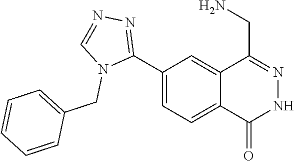 [SMILES: NCc1n[nH]c(=O)c2ccc(-c3nncn3Cc3ccccc3)cc12] | 3A | 3-1 | 4-(aminomethyl)-6-(4-benzyl-4H-1,2,4- triazol-3-yl)phthalazin-1(2H)-one LCMS [M + 1]⁺; 333.1; ¹H NMR (400 MHz, DMSO-d6) δ = 13.08 (s, 1H), 9.05 (s, 1H), 8.59 (br s, 3H), 8.37 (d, J = 8.4 Hz, 1H), 8.17 (s, 1H), 8.14 (dd, J = 1.6, 8.4 Hz, 1H), 7.35- 7.23 (m, 3H), 7.10-7.05 (m, 2H), 5.56 (s, 2H), 4.47-4.35 (m, 2H). |
| 3-58 | 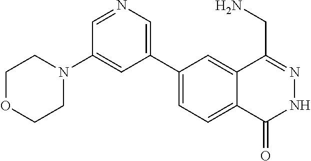 [SMILES: NCc1n[nH]c(=O)c2ccc(-c3cncc(N4CCOCC4)c3)cc12] | 3A | 3-1 | 4-(aminomethyl)-6-(5-morpholinopyridin-3- yl)phthalazin-1(2H)-one LCMS [M + 1]⁺ = 338.1; ¹H NMR (400 MHz, DMSO-d6) δ = 13.03 (s, 1H), 8.71 (s, 1H), 8.53 (br s, 3H), 8.49 (d, J = 2.8 Hz, 1H), 8.43-8.39 (d, J = 8.4 Hz, 1H), 8.38-8.34 (dd, J = 1.6, 8.4 Hz, 1H), 8.34 (s, 1H), 8.17-8.07 (s, 1H), 4.61 (q, J = 6.0 Hz, 2H), 3.83-3.79 (t, J = 4.8 Hz, 4H), 3.47-3.45 (m, 4H) |
| 3-59 | 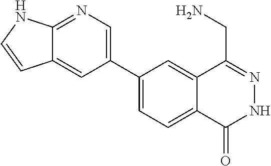 [SMILES: NCc1n[nH]c(=O)c2ccc(-c3cnc4[nH]ccc4c3)cc12] | 3A | 3-2 | 4-(aminomethyl)-6-(1H-pyrrolo[2,3-b]- pyridin-5-yl)phthalazin-1(2H)-one LCMS [M + 1]⁺ = 292.0; ¹H NMR (400 MHz, DMSO-d6) δ = 12.6 (br s, 1H), 11.85 (br s, 1H), 8.73 (d, J = 2.0 Hz, 1H), 8.46 (d, J = 2.0 Hz, 1H), 8.37-8.31 (m, 2H), 8.27-8.17 (m, 1H), 7.57 (dd, J = 2.0, 3.2 Hz, 1H), 6.60-6.54 (m, 1H), 4.20 (s, 2H), 3.88-3.54 (s, 2H) |
| 3-60 | 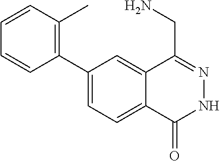 [SMILES: Cc1ccccc1-c1ccc2c(=O)[nH]nc(CN)c2c1] | 3A | 3-1 | 4-(aminomethyl)-6-(o-tolyl)phthalazin- 1(2H)-one LCMS [M + 1]⁺ = 266.1; ¹H NMR (400 MHz, DMSO-d6) δ = 13.0 (s, 1H), 8.39- 8.33 (m, 4H), 7.94-7.90 (m, 2H), 7.40-7.34 (m, 4H), 4.51 (s, 2H), 2.28 (s, 3H) |
| 3-61 | 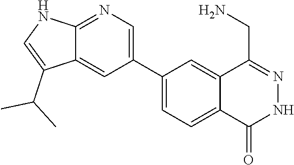 [SMILES: CC(C)c1c[nH]c2ncc(-c3ccc4c(=O)[nH]nc(CN)c4c3)cc12] | 3A | 3-4 | 4-(aminomethyl)-6-(3-isopropyl-1H-pyrrolo- [2,3-b]pyridin-5-yl)phthalazin-1(2H)-one LCMS [M + 1]⁺ = 334.1; ¹H NMR (400 MHz, DMSO-d6) δ = 12.94 (s, 1H), 11.80 (br s, 1H), 8.83 (d, J = 2.0 Hz, 1H), 8.64 (d, J = 2.0 Hz, 1H), 8.60 (br s, 3H), 8.41-8.36 (m, 1H), 8.36-8.31 (m, 1H), 8.29 (s, 1H), 7.36 (d, J = 2.0 Hz, 1H), 4.63 (br d, J = 5.6 Hz, 2H), 3.36-3.19 (m, 1H), 1.37 (d, J = 6.8 Hz, 6H) |

### Examples 4-1 to 4-180

### Coupling Methods (CM) and Purification Methods (PM) for the Preparation of Examples in Table 4

### CM 4λ:

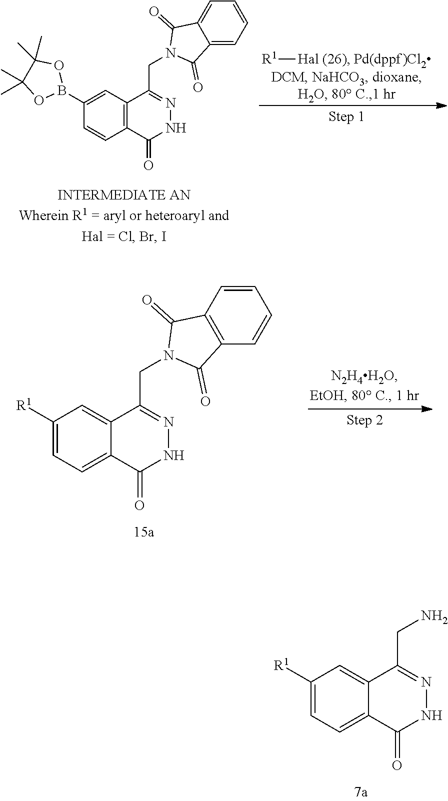

Step 1: A mixture of the appropriate aryl/heteroaryl-halide 26 (390 μmol, 1.5 eq.), intermediate AN (260 μmol, 1.00 eq.), Pd(dppf)Cl₂ (26 μmol, 0.10 eq.), sodium bicarbonate (43.7 mg, 521 μmol, 20.3 μL, 2.00 eq.) in dioxane (1.0 mL) and water (0.2 mL) was purged with nitrogen 3 times. The mixture was then stirred at 80° C. for 2 hours. After such time the mixture was filtered, washed with a dichloromethane:methyl alcohol 10:1 mixture and the filtrate concentrated under reduced pressure to give a residue. This concentrated residue was triturated with methyl alcohol (3.0 mL), filtered and dried to give the corresponding coupling product 15a as a black solid.

Step 2: To a solution of corresponding coupling product 15a in ethyl alcohol (1.0 mL) was added hydrazine hydrate (242 μmol, 14 μL). The mixture was stirred at 80° C. for 1 hour. After such time the mixture was concentrated in vacuo and the residue purified by prep-HPLC according to one of the purification methods 4-1 to 4-25.

### CM 4B:

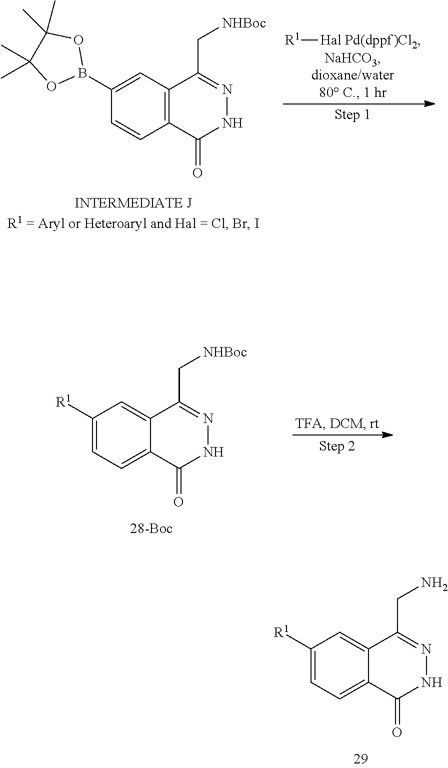

Step 1: Intermediate J (189 mg, 470 μmol, 1.00 eq.), aryl/heteroaryl-halide 26 (564 μmol, 1.20 eq.), Pd(dppf)Cl₂ (34 mg, 47 μmol, 0.10 eq.), sodium bicarbonate (79 mg, 940 μmol, 37 μL, 2.00 eq.) in dioxane (1 mL) and water (0.20 mL) was purged with nitrogen 3 times, and stirred at 80° C. for 2 hours. Upon completion, the reaction mixture was poured into water (40 mL), filtered and the filter cake was dried under reduced pressure to give R²-Pyridyl-Suzuki coupling product 28-Boc (71 mg, crude) as a gray solid and used into the next step directly without further purification.

Step 2: To a solution of tert-butyl N-[[4-oxo-7-(5-pyrimidin-2-yloxy-3-pyridyl)-3H-phthalazin-1-yl]methyl]carbamate R²-Pyridyl-Suzuki coupling product 28-Boc (60 mg, crude) in dichloromethane (1 mL) was added trifluoroacetic acid (462 mg, 4.05 mmol, 0.30 mL). The mixture was stirred at 30° C. for 0.5 hour and upon completion concentrated to a residue under reduced pressure. The concentrated residue 29 was purified by prep-HPLC according to one of the purification methods 4-1 through 4-25. Alternatively, to a solution of tert-butyl N-[[4-oxo-7-(5-pyrimidin-2-yloxy-3-pyridyl)-3H-phthalazin-1-yl]methyl]carbamate R²-Pyridyl-Suzuki coupling product 28-Boc (60 mg, crude) in methanol (1 mL) was added HCl in methanol (4M, 1.0 mL). The mixture was stirred at 25° C. for 0.5 hour and upon completion concentrated to a residue under reduced pressure. The concentrated residue 29 was purified by prep-HPLC according to one of the purification methods 4-1 through 4-25.

### CM 4C

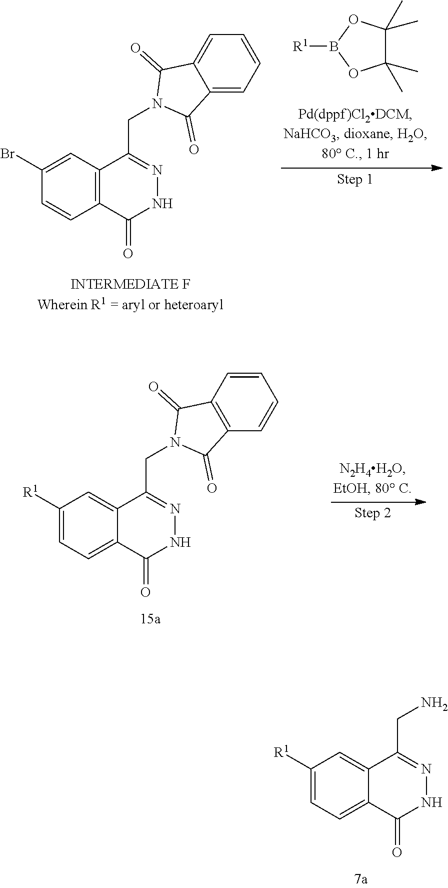

Step 1: A mixture of the appropriate aryl/heteroaryl boronic ester (303 μmol, 1.16 eq.), intermediate F (100 mg, 260 μmol, 1.00 eq.), Pd(dppf)Cl₂ (19 mg, 26 μmol, 0.10 eq.), sodium bicarbonate (66 mg, 781 μmol, 30 μL, 3.00 eq.) in dioxane (1 mL) and water (0.20 mL) was degassed and purged with nitrogen 3 times and stirred at 80° C. for 1 hour under a nitrogen atmosphere. After such time the mixture was filtered and concentrated under reduced pressure and theand the concentrated residue was purified by column chromatography (SiO₂, petroleum ether:ethyl acetate 10:1 to 0:1) to give the corresponding Suzuki coupling product 15a (12.0 mg, crude) as a yellow solid.

Step 2: To a solution of corresponding Suzuki coupling product 15a (10 mg, crude), in ethyl alcohol (1.0 mL) was added hydrazine hydrate (10 mg, 207 μmol, 10 μL). The mixture was stirred at 80° C. for 1 hour, cooled and concentrated under reduced pressure. The concentrated residue 7a was then purified by prep-HPLC according to one of the purification methods 4-1 to 4-25 described herein.

### CM 4D

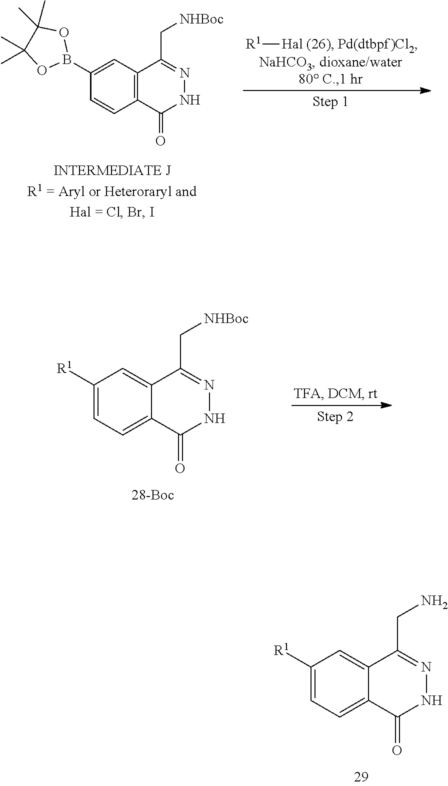

Step 1: Intermediate J (69 mg, 17 μmol, 1.30 eq.), aryl/heteroaryl-halide 26 (132 μmol, 1.0 eq.), Pd(dtbpf)Cl₂ (9 mg, 13 μmol, 0.10 eq.), sodium carbonate (28 mg, 263 μmol, 2.00 eq.) in dioxane (1.5 mL) and water (0.30 mL) was purged with nitrogen 3 times, and stirred at 80° C. for 2 hours. After such time the mixture was concentrated and the residue purified by prep-TLC (SiO₂, CH₂Cl₂:MeOH 20:1) to give R¹-Suzuki coupling product 28-Boc and used into the next step directly without further purification.

Step 2: To a solution of tert-butyl N-[[4-oxo-7-(R¹)-3H-phthalazin-1-yl]methyl]carbamate Suzuki coupling product 28-Boc (40 mg) in dichloromethane (1.5 mL) was added trifluoroacetic acid (5.4 mmol, 0.4 mL). The mixture was stirred at 25° C. for 0.5 hour and upon completion concentrated to a residue under reduced pressure. The concentrated residue 29 was purified by prep-HPLC according to one of the purification methods 4-1 through 4-25.

### CM 4E

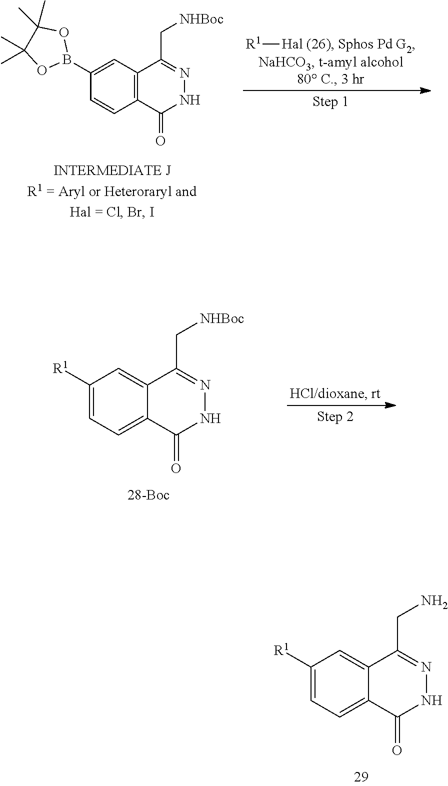

Step 1: Intermediate J (80 mg, 0.20 mmol, 1.00 eq.), aryl/heteroaryl-halide 26 (239 μmol, 1.2 eq.), [2-(2-aminophenyl)phenyl]-chloro-palladium;dicyclohexyl-[2-(2,6-dimethoxyphenyl)phenyl]phosphane (14.4 mg, 19.9 μmol, 0.10 eq.), sodium bicarbonate (34 mg, 398 μmol, 2.00 eq.) in 2-methyl-2-butanol (2.0 mL) and water (0.4 mL) was purged with nitrogen 3 times, and stirred at 80° C. for 3 hours. After such time the mixture was diluted with water (5 mL) and extracted with ethyl acetate (10 mL×3) and the combined organic extracts were dried over anhydrous sodium sulfate, filtered and concentrated under reduced pressure. The residue was then purified by prep-TLC (SiO₂, petroleum ether:ethyl acetate=1:1.5) to give R¹-Suzuki coupling product 28-Boc which was used in the next.

Step 2: A solution of tert-butyl N-[[4-oxo-7-(R¹)-3H-phthalazin-1-yl]methyl]carbamate Suzuki coupling product 28-Boc (0.08 mmol, 1.00 eq) in HCl·dioxane (2.0 mL, 101 eq.) was stirred at 25° C. for 12 hours. The reaction was concentrated in vacuo and the residue purified by prep-HPLC according to one of the purification methods 4-1 through 4-25.

### CM 4F:

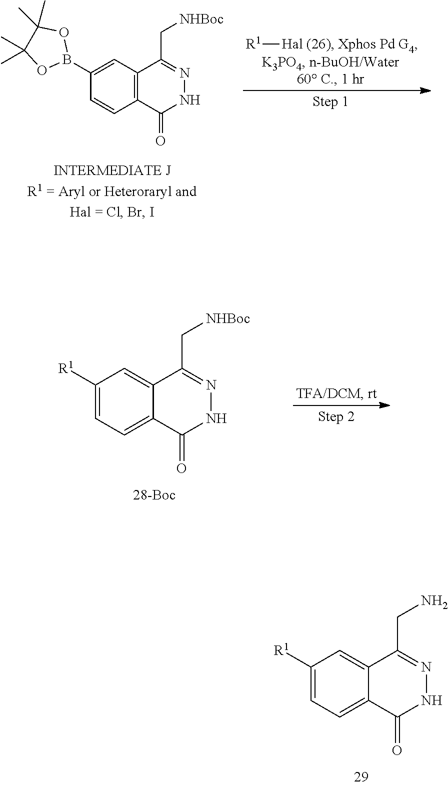

Step 1: Intermediate J (80 mg, 0.20 mmol, 1.00 eq.), aryl/heteroaryl-halide 26 (0.24 mmol, 1.2 eq.), methanesulfonato(2-dicyclohexylphosphino-2′,4′,6′-tri-i-propyl-1,1′-biphenyl)(2′-methylamino-1,1′-biphenyl-2-yl)palladium(II) (20 μmol, 0.10 eq.), potassium phosphate, 0.40 mmol, 2.00 eq.) in n-butanol (2.0 mL) and water (0.4 mL) was purged with nitrogen 3 times, and stirred at 60° C. for 1 hour. After such time the mixture was diluted with water (5 mL) and extracted with ethyl acetate (10 mL×3) and the combined organic extracts were dried over anhydrous sodium sulfate, filtered and concentrated under reduced pressure. The residue was then purified by prep-TLC (SiO₂, petroleum ether ethyl acetate 200%) to give R¹-Suzuki coupling product 28-Boc which was used in the next step.

Step 2: To a solution of tert-butyl N-[[4-oxo-7-(R¹)-3H-phthalazin-1-yl]methyl]carbamate Suzuki coupling product 28-Boc (40.0 mg) in dichloromethane (1.5 mL) was added trifluoroacetic acid (5.4 mmol, 0.40 mL). The mixture was stirred at 25° C. for 0.5 hour and upon completion concentrated to a residue under reduced pressure. The concentrated residue 29 was purified by prep-HPLC according to one of the purification methods 4-1 through 4-25.

### CM 4G:

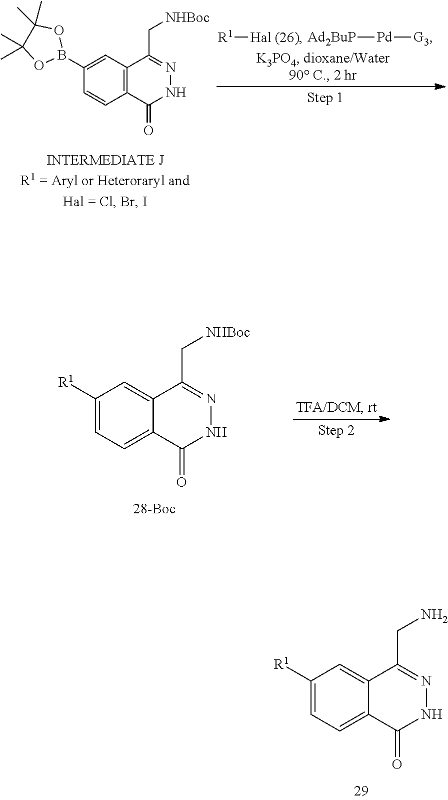

Step 1: Intermediate J (40 mg, 0.10 mmol, 1.25 eq.), aryl/heteroaryl-halide 26 (0.076 mmol, 1.0 eq), [2-(2-aminophenyl)phenyl]palladium(1+);bis(1-adamantyl)-butyl-phosphane methanesulfonate (8 μmol, 0.10 eq.), potassium phosphate, (0.23 mmol, 3.00 eq.) in dioxane (1.5 mL) and water (0.3 mL) was purged with nitrogen 3 times, and stirred at 90° C. for 2 hours. After such time the mixture was filtered, concentrated and the residue was then purified by prep-TLC (SiO₂, petroleum ether:ethyl acetate 60%) to give R¹-Suzuki coupling product 28-Boc which was used in the next step.

Step 2: To a solution of tert-butyl N-[[4-oxo-7-(R¹)-3H-phthalazin-1-yl]methyl]carbamate Suzuki coupling product 28-Boc (30.0 mg) in dichloromethane (2.0 mL) was added trifluoroacetic acid (0.2 mL). The mixture was stirred at 25° C. for 0.5 hour and upon completion concentrated to a residue under reduced pressure. The concentrated residue 29 was purified by prep-HPLC according to one of the purification methods 4-1 through 4-25.

### CM 4H:

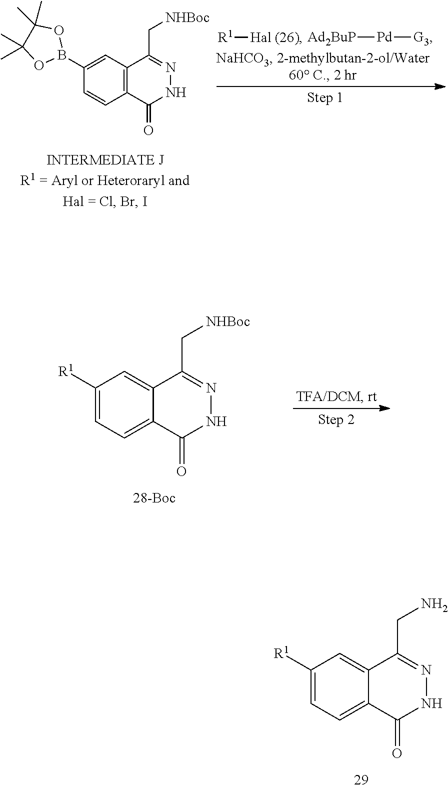

Step 1: Intermediate J (40 mg, 0.10 mmol, 1.0 eq.), aryl/heteroaryl-halide 26 (0.10 mmol, 1.0 eq), (Ad₂ n-BuP)—Pd G3 (8 μmol, 0.10 eq.), potassium phosphate, (0.23 mmol, 3.00 eq.) in dioxane (1.5 mL) and water (0.3 mL) was purged with nitrogen 3 times, and stirred at 90° C. for 2 hours. The mixture was diluted with water (20 mL) and extracted with ethyl acetate (20 mL×3). The combined organic phase was washed with brine (20 mL×3), dried with anhydrous sodium sulfate, filtered and concentrated in vacuum. The residue was purified by prep-TLC (SiO₂, petroleum ether/ethyl acetate 1:1) to give R¹-Suzuki coupling product 28-Boc which was used in the next step.

Step 2: To a solution of tert-butyl N-[[4-oxo-7-(R¹)-3H-phthalazin-1-yl]methyl]carbamate Suzuki coupling product 28-Boc (30.0 mg) in dichloromethane (2.0 mL) was added trifluoroacetic acid (0.2 mL). The mixture was stirred at 25° C. for 0.5 hour and upon completion concentrated to a residue under reduced pressure. The concentrated residue 29 was purified by prep-HPLC according to one of the purification methods 4-1 through 4-23.

### CM 4I:

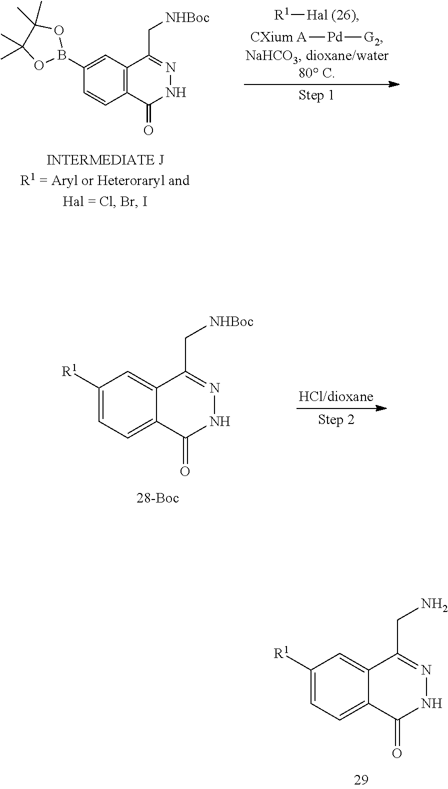

Step 1: Intermediate J (1.0 eq.), aryl/heteroaryl-halide 26 (1.0 eq), [2-(2-aminophenyl)phenyl]palladium(1+):bis(1-adamantyl)-butyl-phosphane;methanesulfonate (8 μmol, 0.20 eq.), sodium bicarbonate, (2.0 eq.) in dioxane (1.5 mL) and water (0.3 mL) was purged with nitrogen 3 times, and stirred at 80° C. for 12 hours. The mixture was then filtered, concentrated and purified by prep-TLC (SiO₂, petroleum ether/EtOAc 1:1) to give R¹-Suzuki coupling product 28-Boc which was used in the next step.

Step 2: To a solution of tert-butyl N-[[4-oxo-7-(R¹)-3H-phthalazin-1-yl]methyl]carbamate Suzuki coupling product 28-Boc (20 mg) in 1,4-dioxane (0.5 mL) was added 2M HCl in 1,4-dioxane (0.5 mL). The mixture was stirred at 20° C. for 1 hour and upon completion concentrated to a residue under reduced pressure. The concentrated residue 29 was purified by one of the purification methods 4-1 through 4-25.

### Purification Methods (PM)

PM 4-1: column: Phenomenex Synergi C18 150×25 mm×10 μm; mobile phase: [water (0.05% HCl)-ACN]; B %: 5/6-30%, 10 min.
PM 4-2: column: Waters Xbridge 150×25 mm×5 μm; mobile phase: [water (0.05% ammonia hydroxide v/v)-ACN]; B %: 5%-40%, 10 min.
PM 4-3: column: Waters Atlantis T3 150×30 mm×5 μm; mobile phase: [water (0.225% FA)-ACN]; B %: 1%-20%, 10 min.
PM 4-4: column: Phenomenex luna C18 150×25 mm×10 μm; mobile phase: [water (0.1% TFA)-ACN]; B %: 1%-30%, 10 min.
PM 4-5: column: Phenomenex Gemini NX—C18 75×30 mm×3 μm; mobile phase: [water (0.05% ammonia hydroxide v/v)-ACN]; B %: 5%-40%, 7 min.
PM 4-6: column: Phenomenex Luna C18 75×30 mm×3 μm; mobile phase: [water (0.05% HCl)-ACN]; B %: 10%-35%, 6.5 min.
PM 4-7: column: Xtimate C18 150×40 mm×10 μm; mobile phase: [water (0.05% ammonia hydroxide v/v)-ACN]; B %: 5%-35%, 10 min.
PM 4-8: column: Waters Xbridge BEH C18 150×25 mm×5 μm; mobile phase: [water (0.05% NH₄HCO₃ v/v)-ACN]; B %: 5%-40%, 10 min.
PM 4-9: column: Nano-micro Kromasil C18 100 mm×40 mm×10 μm; mobile phase: [water (0.1% TFA)-ACN]; B %: 1%-40%, 8 min.
PM 4-10: SFC (column: Daicel ChiralPak IG (250 mm×30 mm, 10 μm); mobile phase: [0.1% NH₃H₂O/MeOH]; B %: 40% isocratic, 7.8 min; 109 min).
PM 4-11: SFC (column: Phenomenex-Cellulose-2 (250 mm×30 mm, 10 μm); mobile phase: [0.1% NH₃H₂O/MeOH]; B %: 50% isocratic, 11.0 min; 95 min).

PM-4-12: The residue was diluted with 50% sodium bicarbonate (20 mL) and extracted with dichloromethane (15 mL×3). The combined organic layers were dried over anhydrous sodium sulfate, filtered and concentrated under reduced pressure to give title compound.
PM 4-13: column: Welch Xtimate C18 150 mm×25 mm×5 μm; mobile phase: [water (0.05% HCl)-ACN]; B %: 10%-40%, 8 min.
PM 4-14: column: Phenomenex Gemini-NX C18 75×30 mm×3 μm; mobile phase: [water (0.1% TFA)-ACN]; B %: 1%-20%, 7 min.
PM 4-15: SFC (column: Chiralpak AS-3 50×4.6 mm I.D., 3 μm; mobile phase: (CO₂-0.05% DEA/MeOH); B %: 5-40% gradient 3 mL/min.
PM 4-16: column: Shim-pack C18 150×25 mm×10 μm; mobile phase:[water (0.225% formic acid)-acetonitrile]; B %: 10%-30%, 10 min
PM 4-17: column: Unisil 3-100 C18 ultra 150×50 mm×3 μm; mobile phase:[water (0.225% formic acid)-acetonotrile]; B %: 1%-30%, 35 min
PM 4-18: column: Phenomenex Luna C18 250×70 mm×10 μm; mobile phase: [water (0.225% formic acid]; B %: 5-40%, 25 min.
PM 4-19: column: Phenomenex Synergi C18 150×25 mm, 10 μm; mobile phase: [water (0.225% formic acid)-ACN]; B %: 15%-45%, 10 min
PM 4-20: The residue was triturated with acetonitrile (15 mL) at 25° C. for 1 hour and the mixture was filtered. The filter cake was dissolved in water (20 mL) and.
PM 4-21: The residue was diluted with methanol (1.0 mL) and water (10.0 mL). The resulting mixture was lyophilized.
PM 4-22: column: Phenomenex Gemini 150*25 mm*10 μm; mobile phase: [water (0.225% formic acid)-ACN]; B %: 5%-35%, 10 min
PM 4-23: The residue was triturated with Petroleum ether (2 mL) and then Ethyl acetate (2 mL), filtered. The filter cake was dissolved in water and lyophilized.
PM 4-24: No further purification.
PM 4-25: column: YMC Triart C118 150×30 mm×7 μm; mobile phase: [water (0.05% HCl)-ACN], B %: 20%-40%, 7 min.

Following the teachings of the General Reaction Schemes, the coupling methods 4A-H, and using purification methods 4-1 to 4-23 and the Intermediates disclosed herein, the Examples 4-1 to 4-286 were prepared as shown in Tables 4A and 4B, or using similar methods:

TABLE 4A

| Exam- | | | | |
|---|---|---|---|---|
| ple | Structure | CM | PM | Compound Name and Characterization |
| 4-1 | 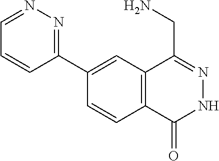 [SMILES: NCc1n[nH]c(=O)c2ccc(-c3cccnn3)cc12] | 4A | 4-1 | 4-(aminomethyl)-6-(pyridazin-3-yl)phthalazin- 1(2H)-one LCMS [M + 1]⁺ = 254.2; ¹H NMR (400 MHz, DMSO-d6) δ = 13.04 (s, 1H), 9.35 (dd, J = 1.6, 5.2 Hz, 1H), 8.71 (dd, J = 1.6, 8.4 Hz, 1H), 8.65 (d, J = 1.2 Hz, 1H), 8.62 (br dd, J = 1.6, 8.8 Hz, 4H), 8.46 (d, J = 8.4 Hz, 1H), 7.95 (dd, J = 4.8, 8.8 Hz, 1H), 4.61 (q, J = 5.2 Hz, 2H) |
| 4-2 | 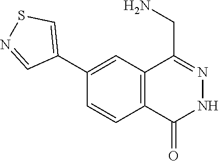 [SMILES: NCc1n[nH]c(=O)c2ccc(-c3cnsc3)cc12] | 4A | 4-1 | 4-(aminomethyl)-6-(isothiazol-4-yl)phthalazin- 1(2H)-one LCMS [M + 1]⁺ = 258.8; ¹H NMR (400 MHz, DMSO-d6) δ = 12.96 (s, 1H), 9.76 (s, 1H), 9.35 (s, 1H), 8.52 (br s, 3H), 8.39-8.34 (m, 3H), 4.56 (br d, J = 5.6 Hz, 2H) |
| 4-3 | 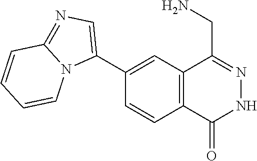 [SMILES: NCc1n[nH]c(=O)c2ccc(-c3cnc4ccccn34)cc12] | 4A | 4-1 | 4-(aminomethyl)-6-(imidazo[1,2-a]pyridin-3- yl)phthalazin-1(2H)-one LCMS [M + 1]⁺ = 292.0; ¹H NMR (400 MHz, DMSO-d6) δ = 13.08 (s, 1H), 9.14 (d, J = 6.8 Hz, 1H), 8.80-8.60 (m, 4H), 8.51-8.39 (m, 2H), 8.24 (dd, J = 1.2, 8.4 Hz, 1H), 8.10 (d, J = 9.2 Hz, 1H), 8.03-7.92 (m, 1H), 7.50 (t, J = 6.4 Hz, 1H), 4.53 (br d, J = 5.6 Hz, 2H) |
| 4-4 | 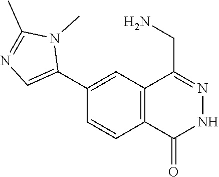 [SMILES: Cc1ncc(-c2ccc3c(=O)[nH]nc(CN)c3c2)n1C] | 4A | 4-1 | 4-(aminomethyl)-6-(1,2-dimethyl-1H-imidazol-5- yl)phthalazin-1(2H)-one LCMS [M + 1]⁺ = 270.1; ¹H NMR (400 MHz, DMSO-d6) δ = 13.07 (s, 1H), 8.69 (br s, 3H), 8.43 (d, J = 8.4 Hz, 1H), 8.20 (d, J = 1.2 Hz, 1H), 8.08 (dd, J = 1.6, 8.4 Hz, 1H), 8.03 (s, 1H), 4.49 (br d, J = 4.8 Hz, 2H), 3.79 (s, 3H), 2.72 (s, 3H) |
| 4-5 | 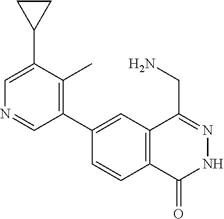 [SMILES: Cc1c(-c2ccc3c(=O)[nH]nc(CN)c3c2)cncc1C1CC1] | 4A | 4-2 | 4-(aminomethyl)-6-(5-cyclopropyl-4- methylpyridin-3-yl)phthalazin-1(2H)-one LCMS [M + 1]⁺ = 307.3; ¹H NMR (400 MHz, DMSO-d6) δ = 8.37-8.28 (m, 3H), 8.05 (d, J = 1.2 Hz, 1H), 7.80 (dd, J = 1.6, 8.0 Hz, 1H), 4.05 (s, 2H), 2.35 (s, 3H), 2.02-1.94 (m, 1H), 1.08- 1.00 (m, 2H), 0.81-0.72 (m, 2H) |
| 4-6 | 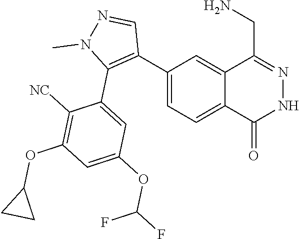 [SMILES: Cn1ncc(-c2ccc3c(=O)[nH]nc(CN)c3c2)c1-c1cc(OC(F)F)cc(OC2CC2)c1C#N] | 4D | 4-6 | 2-(4-(4-(aminomethyl)-1-oxo-1,2- dihydrophthalazin-6-yl)-1-methyl-1H-pyrazol-5- yl)-6-cyclopropoxy-4- (difluoromethoxy)benzonitrile LCMS [M + 1]⁺ = 479.1; ¹H NMR (400 MHz, DMSO-d₆) δ = 12.88 (s, 1H), 8.48 (br s, 3H), 8.29 (s, 1H), 8.13 (d, J = 8.4 Hz, 1H), 7.81-7.39 (m, 4H), 7.21 (d, J = 2.0 Hz, 1H), 4.39-4.13 (m, 3H), 3.75 (s, 3H), 0.98-0.70 (m, 4H). |
| 4-7 | 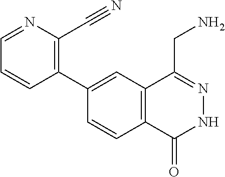 [SMILES: N#Cc1ncccc1-c1ccc2c(=O)[nH]nc(CN)c2c1] | 4A | 4-1 | 3-(4-(aminomethyl)-1-oxo-1,2-dihydrophthalazin- 6-yl)picolinonitrile LCMS [M + 1]⁺ = 278.0; ¹H NMR (400 MHz, DMSO-d6) δ = 13.08 (s, 1H), 8.88 (dd, J = 1.2, 4.4 Hz, 1H), 8.57 (br s, 3H), 8.45 (d, J = 8.4 Hz, 1H), 8.37-8.31 (m, 2H), 8.23 (dd, J = 1.6, 8.0 Hz. 1H), 7.95 (dd, J = 4.8, 8.0 Hz, 1H), 4.52-4.45 (m. 2H) |
| 4-8 | 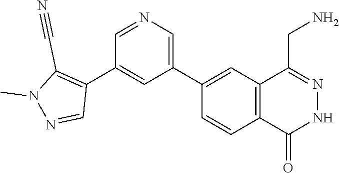 [SMILES: Cn1ncc(-c2cncc(-c3ccc4c(=O)[nH]nc(CN)c4c3)c2)c1C#N] | 4A | 4-1 | 4-(5-(4-(aminomethyl)-1-oxo-1,2- dihydrophthalazin-6-yl)pyridin-3-yl)-1-methyl- 1H-pyrazole-5-carbonitrile LCMS [M + 1]⁺ = 358.1; ¹H NMR (400 MHz, DMSO-d6) δ = 13.03 (s, 1H), 9.19 (d, J = 2.0 Hz, 1H), 9.02 (d, J = 2.0 Hz, 1H), 8.58 (t, J = 2.0 Hz, 1H), 8.50-8.42 (m, 4H), 8.39-8.33 (m, 3H), 4.67-4.55 (s, 2H), 4.13 (s, 3H) |
| 4-9 | 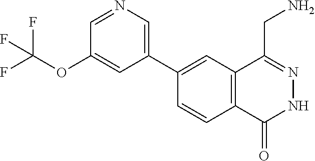 [SMILES: NCc1n[nH]c(=O)c2ccc(-c3cncc(OC(F)(F)F)c3)cc12] | 4A | 4-1 | 4-(aminomethyl)-6-(5-(trifluoromethoxy)pyridin- 3-yl)phthalazin-1(2H)-one LCMS [M + 1]⁺ = 337.0; ¹H NMR (400 MHz, DMSO-d6) δ = 13.02 (s, 1H), 9.25 (d, J = 2.0 Hz, 1H), 8.82 (d, J = 1.6 Hz, 1H), 8.65-8.49 (m, 4H), 8.42-8.38 (m, 1H), 8.38-8.33 (m, 2H), 4.65- 4.54 (m, 2H) |
| 4-10 | 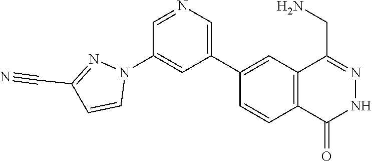 [SMILES: N#Cc1ccn(-c2cncc(-c3ccc4c(=O)[nH]nc(CN)c4c3)c2)n1] | 4A | 4-2 | 1-(5-(4-(aminomethyl)-1-oxo-1,2- dihydrophthalazin-6-yl)pyridin-3-yl)-1H- pyrazole-3-carbonitrile LCMS [M + 1]⁺ = 344.2; ¹H NMR (400 MHz, DMSO-d6) δ = 12.65-12.51 (m, 1H), 9.23 (d, J = 2.4 Hz, 1H), 9.18 (d, J = 2.0 Hz, 1H), 9.02 (d, J = 2.8 Hz, 1H), 8.76 (t, J = 2.0 Hz, 1H), 8.48 (s, 1H), 8.41-8.37 (m, 1H), 8.35-8.30 (m, 1H), 7.40 (d, J = 2.4 Hz, 1H), 4.13 (s, 2H) |
| 4-11 | 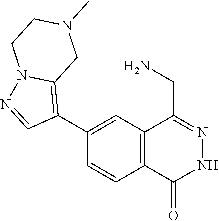 [SMILES: CN1CCn2ncc(-c3ccc4c(=O)[nH]nc(CN)c4c3)c2C1] | 4A | 4-1 | 4-(aminomethyl)-6-(5-methyl-4,5,6,7- tetrahydropyrazolo[1,5-a]pyrazin-3-yl)phthalazin- 1(2H)-one LCMS [M + 1]⁺ = 311.2; ¹H NMR (400 MHz, DMSO-d6) δ = 12.91 (s, 1H), 12.28 (br s, 1H), 8.57 (br s, 3H), 8.36-8.23 (m, 2H), 7.98-7.84 (m, 2H), 4.84 (br s, 2H), 4.54 (br d, J = 4.0 Hz, 4H), 3.74 (br s, 2H), 2.99 (br s, 3H) |
| 4-12 | 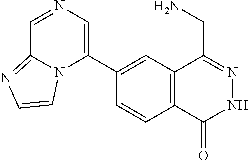 [SMILES: NCc1n[nH]c(=O)c2ccc(-c3cncc4nccn34)cc12] | 4A | 4-1 | 4-(aminomethyl)-6-(imidazo[1,2-a]pyrazin-5- yl)phthalazin-1(2H)-one LCMS [M + 1]⁺ = 293.1; ¹H NMR (400 MHz, DMSO-d6) δ = 13.12 (s, 1H), 9.29-9.23 (s, 1H), 8.52 (br s, 3H), 8.49 (s, 1H), 8.46 (d, J = 1.2 Hz, 1H), 8.39 (s, 1H), 8.33 (dd, J = 1.6, 8.4 Hz, 1H), 8.23 (s, 1H), 8.04-8.01 (m, 1H), 4.59-4.51 (d, J = 5.6 Hz, 2H) |
| 4-13 | 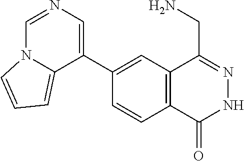 [SMILES: NCc1n[nH]c(=O)c2ccc(-c3cncn4cccc34)cc12] | 4A | 4-1 | 4-(aminomethyl)-6-(pyrrolo[1,2-c]pyrimidin-4- yl)phthalazin-1(2H)-one LCMS [M + 1]⁺ = 292.1; ¹H NMR (400 MHz, DMSO-d6) δ = 13.00 (s, 1H), 9.41 (s, 1H), 8.62 (br s, 3H), 8.42 (d, J = 8.4 Hz, 1H), 8.30-8.25 (m, 2H), 7.90 (dd, J = 1.2, 2.8 Hz, 1H), 7.78 (s, 1H), 7.06 (dd, J = 2.8, 3.6 Hz, 1H), 6.83 (d, J = 3.6 Hz, 1H), 4.57 (q, J = 5.4 Hz, 2H) |
| 4-14 | 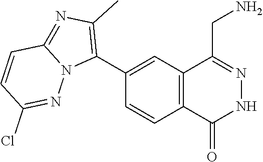 [SMILES: Cc1nc2ccc(Cl)nn2c1-c1ccc2c(=O)[nH]nc(CN)c2c1] | 4A | 4-1 | 4-(aminomethyl)-6-(6-chloro-2- methylimidazo[1,2-b]pyridazin-3-yl)phthalazin- 1(2H)-one LCMS [M + 1]⁺ = 341.1 ¹H NMR (400 MHz, DMSO-d6) δ = 13.02 (s, 1H), 8.62 (br s, 3H), 8.45 (d, J = 8.4 Hz, 1H), 8.31 (d, J = 9.6 Hz, 1H), 8.29-8.22 (m, 2H), 7.56 (d, J = 9.2 Hz, 1H), 4.47 (br d, J = 5.6 Hz, 2H), 2.61 (s, 3H) |
| 4-15 | 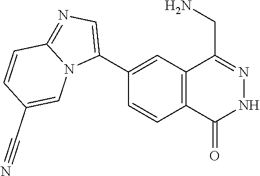 [SMILES: N#Cc1ccc2ncc(-c3ccc4c(=O)[nH]nc(CN)c4c3)n2c1] | 4A | 4-2 | 3-(4-(aminomethyl)-1-oxo-1,2-dihydrophthalazin- 6-yl)imidazo[1,2-a]pyridine-6-carbonitrile LCMS [M + 1]⁺ = 317.2; ¹H NMR (400 MHz, DMSO-d6) δ = 12.56 (s, 1H), 9.53-9.43 (m, 1H), 8.42-8.36 (m, 2H), 8.23-8.12 (m, 2H), 7.88 (dd, J = 0.8, 9.2 Hz, 1H), 7.63 (dd, J = 1.2, 9.2 Hz, 1H), 4.10 (s, 2H) |
| 4-16 | 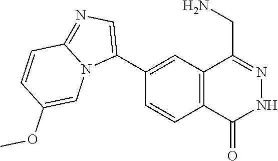 [SMILES: COc1ccc2ncc(-c3ccc4c(=O)[nH]nc(CN)c4c3)n2c1] | 4A | 4-2 | 4-(aminomethyl)-6-(6-methoxyimidazo[1,2- a]pyridin-3-yl)phthalazin-1(2H)-one LCMS [M + 1]⁺ = 322.1; ¹H NMR (400 MHz, DMSO-d6) δ = 12.51 (br s, 1H), 8.47 (s, 1H), 8.36 (d, J = 8.0 Hz, 1H), 8.26 (s, 1H), 8.13 (d, J = 8.4 Hz, 1H), 7.97 (s, 1H), 7.65 (d, J = 10.0 Hz, 1H), 7.17 (dd, J = 2.0, 9.6 Hz, 1H), 4.07 (s, 2H), 3.83 (s, 3H) |
| 4-17 | 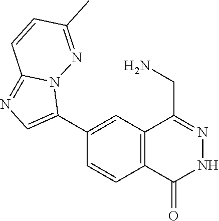 [SMILES: Cc1ccc2ncc(-c3ccc4c(=O)[nH]nc(CN)c4c3)n2n1] | 4A | 4-1 | 4-(aminomethyl)-6-(6-methylimidazo[1,2- b]pyridazin-3-yl)phthalazin-1(2H)-one LCMS [M + 1]⁺ = 307.0; ¹H NMR (400 MHz, DMSO-d6) δ = 12.98 (s, 1H), 8.87 (s, 1H), 8.75 (dd, J = 1.2, 8.4 Hz, 1H), 8.71 (d, J = 1.2 Hz, 1H), 8.69 (br s, 3H), 8.42 (d, J = 8.4 Hz, 1H), 8.36 (d, J = 9.6 Hz, 1H), 7.58 (d, J = 9.6 Hz, 1H), 4.58- 4.51 (m, 2H), 2.71 (s, 3H). |
| 4-18 | 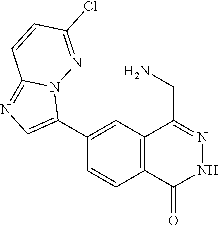 [SMILES: NCc1n[nH]c(=O)c2ccc(-c3cnc4ccc(Cl)nn34)cc12] | 4A | 4-1 | 4-(aminomethyl)-6-(6-chloroimidazo[1,2- b]pyridazin-3-yl)phthalazin-1(2H)-one LCMS [M + 1]⁺ = 327.0; ¹H NMR (400 MHz, DMSO-d6) δ = 12.97 (s, 1H), 8.74 (s, 1H), 8.71- 8.61 (m, 4H), 8.59 (d, J = 1.2 Hz, 1H), 8.42 (m, 2H), 7.58 (d, J = 9.6 Hz, 1H), 4.59-4.47 (s, 2H). |
| 4-19 | 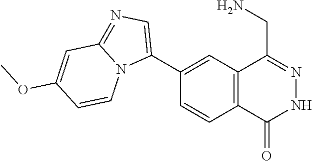 [SMILES: COc1ccn2c(-c3ccc4c(=O)[nH]nc(CN)c4c3)cnc2c1] | 4A | 4-2 | 4-(aminomethyl)-6-(7-methoxyimidazo[1,2- a]pyridin-3-yl)phthalazin-1(2H)-one LCMS [M + 1]⁺ = 322.0; ¹H NMR (400 MHz, DMSO-d6) δ = 12.57 (br s, 1H), 8.72 (d, J = 7.6 Hz, 1H), 8.39 (d, J = 8.4 Hz, 1H), 8.34 (d, J = 1.2 Hz, 1H), 8.14 (dd, J = 1.6, 8.4 Hz, 1H), 7.95 (s, 1H), 7.16 (d, J = 2.4 Hz, 1H), 6.81 (dd, J = 2.8, 7.6 Hz, 1H), 4.14 (s, 2H), 3.95 (s, 3H) |
| 4-20 | 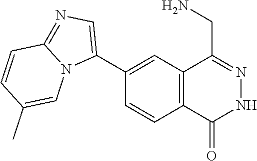 [SMILES: Cc1ccc2ncc(-c3ccc4c(=O)[nH]nc(CN)c4c3)n2c1] | 4A | 4-2 | 4-(aminomethyl)-6-(6-methylimidazo[1,2- a]pyridin-3-yl)phthalazin-1(2H)-one LCMS [M + 1]⁺ = 306.2; ¹H NMR (400 MHz, DMSO-d6) δ = 12.52 (br s, 1H), 8.60 (s, 1H), 8.43- 8.31 (m, 2H), 8.21-8.06 (m, 1H), 8.03-7.89 (m, 1H), 7.64 (d, J = 9.2 Hz, 1H), 7.26 (dd, J = 1.2, 9.2 Hz, 1H), 4.09 (s, 2H), 2.36-2.33 (s, 3H) |
| 4-21 | 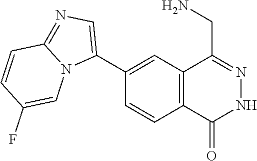 [SMILES: NCc1n[nH]c(=O)c2ccc(-c3cnc4ccc(F)cn34)cc12] | 4A | 4-1 | 4-(aminomethyl)-6-(6-fluoroimidazo[1,2- a]pyridin-3-yl)phthalazin-1(2H)-one LCMS [M + 1]⁺ = 310.0; ¹H NMR (400 MHz, DMSO-d6) δ = 13.04-13.01 (s, 1H), 9.20-9.16 (m, 1H), 8.45 (br d, J = 0.8 Hz, 3H), 8.43 (d, J = 8.4 Hz, 1H), 8.29 (m, 2H), 8.22 (dd, J = 1.6, 8.4 Hz, 1H), 7.96-7.89 (m, 1H), 7.74-7.64 (m, 1H), 4.61-4.55 (m, 2H) |
| 4-22 | 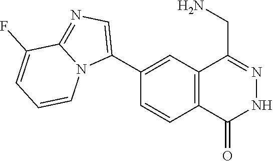 [SMILES: NCc1n[nH]c(=O)c2ccc(-c3cnc4c(F)cccn34)cc12] | 4A | 4-1 | 4-(aminomethyl)-6-(8-fluoroimidazo[1,2- a]pyridin-3-yl)phthalazin-1(2H)-one LCMS [M + 1]⁺ = 310.0; ¹H NMR (400 MHz, DMSO-d6) δ = 13.03 (s, 1H), 8.81 (d, J = 6.8 Hz, 1H), 8.58 (br s, 3H), 8.44 (d, J = 8.4 Hz, 1H), 8.30 (s, 1H), 8.27-8.19 (m, 2H), 7.44 (dd, J = 7.6, 10.8 Hz, 1H), 7.11 (dt, J = 5.2, 7.2 Hz, 1H), 4.76- 4.34 (m, 2H) |
| 4-23 | 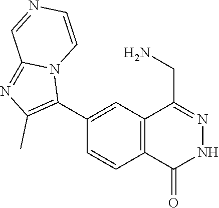 [SMILES: Cc1nc2cnccn2c1-c1ccc2c(=O)[nH]nc(CN)c2c1] | 4A | 4-1 | 4-(aminomethyl)-6-(2-methylimidazo[1,2- a]pyrazin-3-yl)phthalazin-1(2H)-one LCMS [M + 1]⁺ = 307.0; ¹H NMR (400 MHz, DMSO-d6) δ = 13.08 (s, 1H), 9.41-9.25 (m, 1H), 8.90-8.79 (m, 1H), 8.71 (s, 3H), 8.48 (d, J = 8.4 Hz, 1H), 8.33 (br s, 1H), 8.14 (dd, J = 1.2, 8.2 Hz, 1H), 8.12-8.04 (m, 1H), 4.52 (br d, J = 5.6 Hz, 2H), 2.59 (s, 3H) |
| 4-24 | 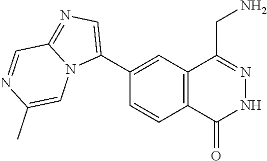 [SMILES: Cc1cn2c(-c3ccc4c(=O)[nH]nc(CN)c4c3)cnc2cn1] | 4A | 4-1 | 4-(aminomethyl)-6-(6-methylimidazo[1,2- a]pyrazin-3-yl)phthalazin-1(2H)-one LCMS [M + 1]⁺ = 307.1 ¹H NMR (400 MHz, DMSO-d6) δ = 13.05 (s, 1H), 9.25 (d, J = 0.8 Hz, 1H), 8.89 (s, 1H), 8.61 (br s, 3H), 8.45 (d, J = 8.4 Hz, 1H), 8.40 (s, 1H), 8.36 (s, 1H), 8.27 (dd, J = 1.2, 8.4 Hz, 1H), 4.65-4.52 (d, J = 5.6 Hz, 2H), 2.54 (s, 3H) |
| 4-25 | 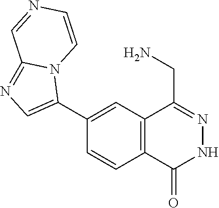 [SMILES: NCc1n[nH]c(=O)c2ccc(-c3cnc4cnccn34)cc12] | 4A | 4-2 | 4-(aminomethyl)-6-(imidazo[1,2-a]pyrazin-3- yl)phthalazin-1(2H)-one LCMS [M + 1]⁺ = 293.2; ¹H NMR (400 MHz, DMSO-d6) δ = 9.17 (d, J = 1.6 Hz, 1H), 8.71 (dd, J = 1.6, 4.8 Hz, 1H), 8.45-8.39 (m, 2H), 8.24 (s, 1H), 8.16 (dd, J = 1.6, 8.4 Hz, 1H), 8.00 (d, J = 4.8 Hz, 1H), 4.12 (s, 2H) |
| 4-26 | 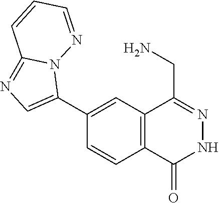 [SMILES: NCc1n[nH]c(=O)c2ccc(-c3cnc4cccnn34)cc12] | 4A | 4-1 | 4-(aminomethyl)-6-(imidazo[1,2-b]pyridazin-3- yl)phthalazin-1(2H)-one LCMS [M + 1]⁺ = 293.2; ¹H NMR (400 MHz, DMSO-d6) δ = 12.98 (s, 1H), 8.87 (dd, J = 1.6, 4.4 Hz, 1H), 8.84 (s, 1H), 8.79 (dd, J = 1.6, 8.4 Hz, 1H), 8.64 (br d, J = 1.2 Hz, 4H), 8.47-8.39 (m, 2H), 7.57 (dd, J = 4.4, 9.2 Hz, 1H), 4.56 (br d, J = 5.6 Hz, 2H) |
| 4-27 | 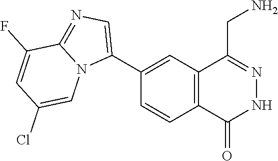 [SMILES: NCc1n[nH]c(=O)c2ccc(-c3cnc4c(F)cc(Cl)cn34)cc12] | 4A | 4-1 | 4-(aminomethyl)-6-(6-chloro-8- fluoroimidazo[1,2-a]pyridin-3-yl)phthalazin- 1(2H)-one LCMS [M + 1]⁺ = 344.0; ¹H NMR (400 MHz, DMSO-d6) δ = 13.03 (s, 1H), 8.93 (d, J = 1.6 Hz, 1H), 8.55 (br s, 3H), 8.43 (d. J = 8.4 Hz, 1H), 8.31 (d, J = 1.2 Hz, 1H), 8.22 (dd, J = 1.6, 8.4 Hz, 1H), 8.17 (s, 1H), 7.65 (dd, J = 1.6, 10.8 Hz, 1H), 4.63- 4.42 (d, J = 5.6 Hz, 2H) |
| 4-28 | 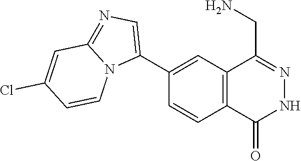 [SMILES: NCc1n[nH]c(=O)c2ccc(-c3cnc4cc(Cl)ccn34)cc12] | 4A | 4-1 | 4-(aminomethyl)-6-(7-chloroimidazo[1,2- a]pyridin-3-yl)phthalazin-1(2H)-one LCMS [M + 1]⁺ = 326.0 ¹H NMR (400 MHz, DMSO-d6) δ = 13.02 (s, 1H), 8.92 (d, J = 7.2 Hz, 1H), 8.43-8.41 (d, J = 8.4 Hz, 1H), 8.40 (s, 3H), 8.23 (s, 1H), 8.23-8.20 (dd, J = 1.2, 8.4 Hz, 1H), 8.11 (s, 1H), 7.94 (d, J = 1.6 Hz, 1H), 7.10 (dd, J = 2.0, 7.2 Hz, 1H), 4.58 (br d, J = 2.0 Hz, 2H) |
| 4-29 | 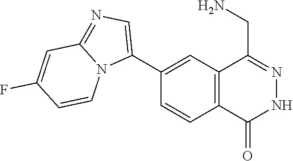 [SMILES: NCc1n[nH]c(=O)c2ccc(-c3cnc4cc(F)ccn34)cc12] | 4A | 4-1 | 4-(aminomethyl)-6-(7-fluoroimidazo[1,2- a]pyridin-3-yl)phthalazin-1(2H)-one LCMS [M + 1]⁺ = 310.0 ¹H NMR (400 MHz, DMSO-d6) δ = 12.80 (br s, 1H), 8.92 (br t, J = 6.4 Hz, 1H), 8.53 (m, 1H), 8.50-8.43 (brs, 3H), 8.25 (s, 1H), 8.22-8.17 (m, 1H), 8.15 (s, 1H), 7.65-7.60 (m, 1H), 7.15 (br t, J = 7.3 Hz, 1H), 4.53 (br s, 2H) |
| 4-30 | 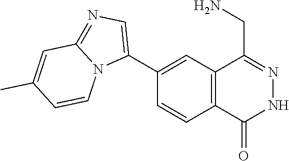 [SMILES: Cc1ccn2c(-c3ccc4c(=O)[nH]nc(CN)c4c3)cnc2c1] | 4A | 4-1 | 4-(aminomethyl)-6-(7-methylimidazo[1,2- a]pyridin-3-yl)phthalazin-1(2H)-one LCMS [M + 1]⁺ = 306.1; ¹H NMR (400 MHz, DMSO-d6) δ = 12.59-12.48 (s, 1H), 8.71 (d, J = 7.2 Hz, 1H), 8.34 (d, J = 8.4 Hz, 1H), 8.31 (d, J = 1.2 Hz, 1H), 8.11 (dd, J = 1.6, 8.4 Hz, 1H), 7.97 (s, 1H), 7.50 (s, 1H), 6.90 (dd, J = 1.6, 7.2 Hz, 1H), 4.11-4.05 (s, 2H), 2.41 (s, 3H) |
| 4-31 | 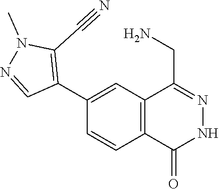 [SMILES: Cn1ncc(-c2ccc3c(=O)[nH]nc(CN)c3c2)c1C#N] | 4A | 4-1 | 4-(4-(aminomethyl)-1-oxo-1,2-dihydrophthalazin- 6-yl)-1-methyl-1H-pyrazole-5-carbonitrile LCMS [M + 1]⁺ = 281.1; ¹H NMR (400 MHz, DMSO-d6) δ = 13.00 (s, 1H), 8.50 (br s, 3H), 8.45 (s, 1H), 8.43-8.40 (d, J = 8.8 Hz, 1H), 8.23 (s, 1H), 8.22-8.20 (dd, J = 1.6, 8.8 Hz 1H), 4.51 (br d, J = 5.6 Hz, 2H), 4.12 (s, 3H) |
| 4-32 | 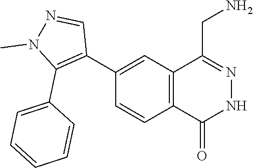 [SMILES: Cn1ncc(-c2ccc3c(=O)[nH]nc(CN)c3c2)c1-c1ccccc1] | 4A | 4-1 | 4-(aminomethyl)-6-(1-methyl-5-phenyl-1H- pyrazol-4-yl)phthalazin-1(2H)-one LCMS [M + 1]⁺ = 332.0; ¹H NMR (400 MHz, DMSO-d6) δ = 12.82 (s, 1H), 8.45 (br s, 3H), 8.386 (s, 1H), 8.12 (d, J = 8.8 Hz, 1H), 7.7 (s, 1H), 7.6 (m, 3H), 7.501 (s, 1H), 7.437 (m, 2H), 4.23 (bs, 2H), 3.74 (s, 3H) |
| 4-33 | 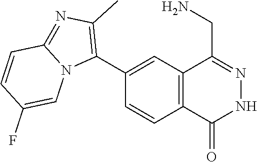 [SMILES: Cc1nc2ccc(F)cn2c1-c1ccc2c(=O)[nH]nc(CN)c2c1] | 4A | 4-2 | 4-(aminomethyl)-6-(6-fluoro-2- methylimidazo[1,2-a]pyridin-3-yl)phthalazin- 1(2H)-one LCMS [M + 1]⁺ = 324.0; ¹H NMR (400 MHz, DMSO-d6) δ = 11.70-11.56 (s, 1H), 7.70 (dd, J = 2.0, 4.8 Hz, 1H), 7.46 (d, J = 8.4 Hz, 1H), 7.35 (d, J = 1.2 Hz, 1H), 7.08 (dd, J = 1.6, 8.4 Hz, 1H), 6.73 (dd, J = 5.2, 9.8 Hz, 1H), 6.51-6.43 (m, 1H), 3.12 (s, 2H), 1.52 (s, 3H) |
| 4-34 | 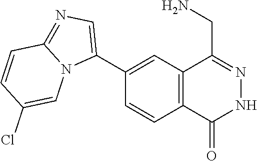 [SMILES: NCc1n[nH]c(=O)c2ccc(-c3cnc4ccc(Cl)cn34)cc12] | 4A | 4-4 | 4-(aminomethyl)-6-(6-chloroimidazo[1,2- a]pyridin-3-yl)phthalazin-1(2H)-one LCMS [M + 1]⁺ = 326.1; ¹H NMR (400 MHz, DMSO-d6) δ = 13.01 (s, 1H), 9.04 (d, J = 1.2 Hz, 1H), 8.45-8.42 (m, 1H), 8.42-8.35 (s, 3H), 8.30- 8.21 (m, 2H), 8.16-8.13 (m, 1H), 7.82 (d, J = 9.2 Hz, 1H), 7.50 (dd, J = 2.0, 9.6 Hz, 1H), 4.59 (q, J = 5.2 Hz, 2H) |
| 4-35 | 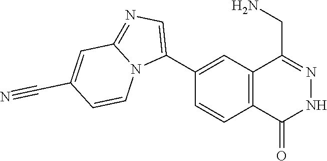 [SMILES: N#Cc1ccn2c(-c3ccc4c(=O)[nH]nc(CN)c4c3)cnc2c1] | 4B | 4-1 | 3-(4-(aminomethyl)-1-oxo-1,2-dihydrophthalazin- 6-yl)imidazo[1,2-a]pyridine-7-carbonitrile LCMS [M + 1]⁺ = 317.0; ¹H NMR (400 MHz, DMSO-d6) δ = 13.05 (s, 1H), 9.13 (d, J = 6.8 Hz, 1H), 8.68 (br s, 3H), 8.59 (s, 1H), 8.49 (s, 1H), 8.43 (d, J = 8.4 Hz, 1H), 8.35 (d, J = 1.2 Hz, 1H), 8.23 (dd, J = 1.2, 8.4 Hz, 1H), 7.45 (dd, J = 1.6, 7.2 Hz, 1H), 4.55 (br d, J = 5.6 Hz, 2H), 2.54 (s, 1H) |
| 4-36 | 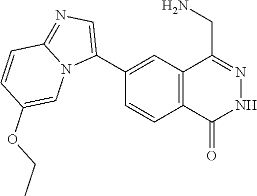 [SMILES: CCOc1ccc2ncc(-c3ccc4c(=O)[nH]nc(CN)c4c3)n2c1] | 4A | 4-2 | 4-(aminomethyl)-6-(6-ethoxyimidazo[1,2- a]pyridin-3-yl)phthalazin-1(2H)-one LCMS [M + 1]⁺ = 336.1; ¹H NMR (400 MHz, DMSO-d6) δ = 12.84 (s, 1H), 8.40 (d, J = 8.4 Hz, 1H), 8.32 (s, 1H), 8.28 (d, J = 2.4 Hz, 1H), 8.21 (d, J = 8.2 Hz, 1H), 7.99 (s, 1H), 7.67 (d, J = 9.6 Hz, 1H), 7.20 (dd, J = 2.4, 9.6 Hz, 1H), 4.42 (br s, 2H), 4.09 (q, J = 7.2 Hz, 2H), 1.37 (t, J = 7.2 Hz, 3H) |
| 4-37 | 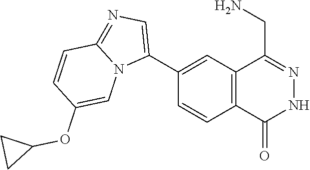 [SMILES: NCc1n[nH]c(=O)c2ccc(-c3cnc4ccc(OC5CC5)cn34)cc12] | 4A | 4-5 | 4-(aminomethyl)-6-(6-cyclopropoxyimidazo[1,2- a]pyridin-3-yl)phthalazin-1(2H)-one LCMS [M + 1]⁺ = 348.1; ¹H NMR (400 MHz, DMSO-d6) δ = 12.81 (s, 1H), 8.51 (d, J = 2.0 Hz, 1H), 8.41 (d, J = 8.4 Hz, 1H), 8.35 (d, J = 1.2 Hz, 1H), 8.22 (dd, J = 1.6, 8.4 Hz, 1H), 8.03 (s, 1H), 7.68 (d, J = 9.6 Hz, 1H), 7.20 (dd, J = 2.0, 9.6 Hz, 1H), 6.87-6.32 (br s, 2H), 4.37 (d, J = 5.6 Hz, 1H), 4.00 (tt, J = 3.2, 5.6 Hz, 1H), 0.83-0.74 (m, 4H). |
| 4-38 | 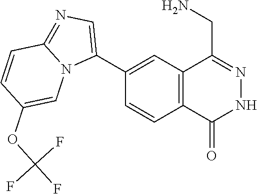 [SMILES: NCc1n[nH]c(=O)c2ccc(-c3cnc4ccc(OC(F)(F)F)cn34)cc12] | 4A | 4-1 | 4-(aminomethyl)-6-(6- (trifluoromethoxy)imidazo[1,2-a]pyridin-3- yl)phthalazin-1(2H)-one LCMS [M + 1]⁺ = 376.0; ¹H NMR (400 MHz, DMSO-d6) δ = 13.05 (s, 1H), 9.34 (s, 1H), 8.66 (br s, 3H), 8.52 (s, 1H), 8.49-8.43 (dd, J = 2.0, 8.4 Hz 1H), 8.38 (s, 1H), 8.21 (d, J = 8.4 Hz, 1H), 8.09 (d, J = 9.6 Hz, 1H), 7.89 (d, J = 9.6 Hz, 1H), 4.52 (br d, J = 5.2 Hz, 2H). |
| 4-39 | 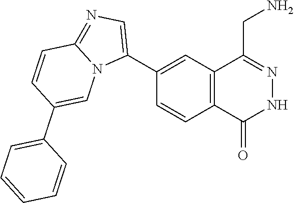 [SMILES: NCc1n[nH]c(=O)c2ccc(-c3cnc4ccc(-c5ccccc5)cn34)cc12] | 4A | 4-5 | 4-(aminomethyl)-6-(6-phenylimidazo[1,2- a]pyridin-3-yl)phthalazin-1(2H)-one LCMS [M + 1]⁺ = 368.1; ¹H NMR (400 MHz, DMSO-d6) δ = 13.05 (s, 1H), 9.01 (s, 1H), 8.61- 8.38 (m, 5H), 8.36-8.29 (m, 2H), 8.02 (m, 2H), 7.84-7.77 (m, 2H), 7.57-7.49 (m, 2H), 7.48- 7.42 (m, 1H), 4.62-4.55 (s, 2H) |
| 4-40 | 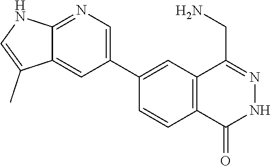 [SMILES: Cc1c[nH]c2ncc(-c3ccc4c(=O)[nH]nc(CN)c4c3)cc12] | 4A | 4-1 | 4-(aminomethyl)-6-(3-methyl-1H-pyrrolo[2,3- b]pyridin-5-yl)phthalazin-1(2H)-one LCMS [M + 1]⁺ = 306.1; ¹H NMR (400 MHz, DMSO-d6) δ = 12.93 (s, 1H), 11.69 (br s, 1H), 8.82 (d, J = 2.0 Hz, 1H), 8.57 (br s, 4H), 8.39- 8.37 (m, 1H), 8.36-8.34 (s, 1H), 8.29 (s, 1H), 7.37 (s, 1H), 4.65-4.59 (d, J = 5.6 Hz, 2H), 2.36 (s, 3H) |
| 4-41 | 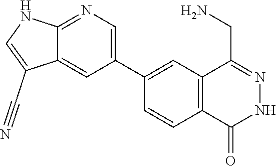 [SMILES: N#Cc1c[nH]c2ncc(-c3ccc4c(=O)[nH]nc(CN)c4c3)cc12] | 4A | 4-1 | 5-(4-(aminomethyl)-1-oxo-1,2-dihydrophthalazin- 6-yl)-1H-pyrrolo[2,3-b]pyridine-3-carbonitrile LCMS [M + 1]⁺ = 317.1 ¹H NMR (400 MHz, DMSO-d6) δ = 13.12-13.06 (s, 1H), 12.97 (s, 1H), 8.97 (d, J = 2.0 Hz, 1H), 8.71 (d, J = 2.0 Hz, 1H), 8.57 (d, J = 2.8 Hz, 1H), 8.49 (br s, 3H), 8.39 (m, 2H), 8.34 (s, 1H), 4.71- 4.59 (d, J = 5.6 Hz, 2H) |
| 4-42 | 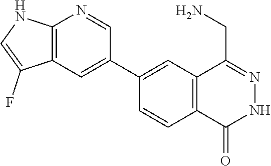 [SMILES: NCc1n[nH]c(=O)c2ccc(-c3cnc4[nH]cc(F)c4c3)cc12] | 4A | 4-4 | 4-(aminomethyl)-6-(3-fluoro-1H-pyrrolo[2,3- b]pyridin-5-yl)phthalazin-1(2H)-one LCMS [M + 1]⁺ = 310.0; ¹H NMR (400 MHz, DMSO-d6) δ = 12.95 (s, 1H), 11.75-11.72 (s, 1H), 8.85 (d, J = 2.0 Hz, 1H), 8.59 (d, J = 2.0 Hz, 1H), 8.39 (br s, 3H), 8.37 (m, 1H), 8.36 (m, 1H), 8.29 (s, 1H), 7.59 (t, J = 2.4 Hz, 1H), 4.63 (br d, J = 5.6 Hz, 2H) |
| 4-43 | 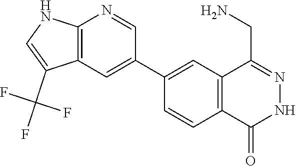 [SMILES: NCc1n[nH]c(=O)c2ccc(-c3cnc4[nH]cc(C(F)(F)F)c4c3)cc12] | 4A | 4-1 | 4-(aminomethyl)-6-(3-(trifluoromethyl)-1H- pyrrolo[2,3-b]pyridin-5-yl)phthalazin-1(2H)-one LCMS [M + 1]⁺ = 360.0; ¹H NMR (400 MHz, DMSO-d6) δ = 12.97 (s, 1H), 12.77 (s, 1H), 8.94 (d, J = 2.0 Hz, 1H), 8.52 (s, 1H), 8.47 (s, 3H), 8.40- 8.36 (m, 1H), 8.36-8.31 (m, 1H), 8.28 (m, 2H), 4.65 (d, J = 5.6 Hz, 2H) |
| 4-44 | 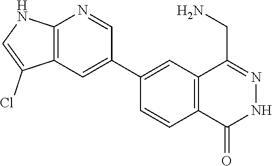 [SMILES: NCc1n[nH]c(=O)c2ccc(-c3cnc4[nH]cc(Cl)c4c3)cc12] | 4A | 4-2 | 4-(aminomethyl)-6-(3-chloro-1H-pyrrolo[2,3- b]pyridin-5-yl)phthalazin-1(2H)-one LCMS [M + 1]⁺ = 326.0; ¹H NMR (400 MHz, DMSO-d6) δ = 12.95 (s, 1H), 12.23 ( s, 1H), 8.88 (d, J = 2.4 Hz, 1H), 8.47 (d, J = 1.6 Hz, 1H), 8.46- 8.38 (s, 3H), 8.38-8.34 (m, 2H), 8.30 (s, 1H), 7.81 (d, J = 2.8 Hz, 1H), 4.65 (br d, J = 4.0 Hz, 2H) |
| 4-45 | 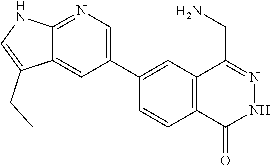 [SMILES: CCc1c[nH]c2ncc(-c3ccc4c(=O)[nH]nc(CN)c4c3)cc12] | 4A | 4-5 | 4-(aminomethyl)-6-(3-ethyl-1H-pyrrolo[2,3- b]pyridin-5-yl)phthalazin-1(2H)-one LCMS [M + 1]⁺ = 320.1; ¹H NMR (400 MHz, DMSO-d6) δ = 12.95-12.88 (s, 1H), 11.59- 11.49 (s, 1H), 8.76 (d, J = 2.0 Hz, 1H), 8.46 (d, J = 2.0 Hz, 1H), 8,43-8.36 (br s, 3H), 8.35-8.30 (m, 2H), 8.23 (s, 1H), 7.33 (s, 1H), 4.71-4.57 (m, 2H), 2.79 (q, J = 7.2 Hz, 2H), 1.34-1.27 (q, J = 7.6 Hz, 3H) |
| 4-46 | 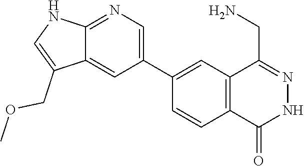 | 4A | 4-2 | 4-(aminomethyl)-6-(3-(methoxymethyl)-1H- pyrrolo[2,3-b]pyridin-5-yl)phthalazin-1(2H)-one LCMS [M + 1]⁺ = 366.1; ¹H NMR (400 MHz, DMSO-d6) δ = 12.75-12.60 (s, 1H), 11.86- 11.78 (s, 1H), 8.77 (d, J = 2.4 Hz, 1H), 8.46 (d, J = 2.4 Hz, 1H), 8.38-8.34 (m, 1H), 8.31 (d, J = 1.2 Hz, 1H), 8.27-8.23 (m, 1H), 7.59 (d, J = 2.0 Hz, 1H), 4.65 (s, 2H), 4.34 (s, 2H), 3.28 (s, 3H) |
| 4-47 | 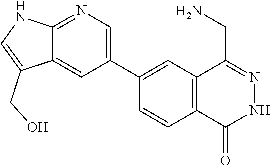 [SMILES: NCc1n[nH]c(=O)c2ccc(-c3cnc4[nH]cc(CO)c4c3)cc12] | 4A | 4-5 | 4-(aminomethyl)-6-(3-(hydroxymethyl)-1H- pyrrolo[2,3-b]pyridin-5-yl)phthalazin-1(2H)-one LCMS [M + 1]⁺ = 322.3; ¹H NMR (400 MHz, DMSO-d6) δ = 8.68 (d, J = 2.0 Hz, 1H), 8.44 (d, J = 2.4 Hz, 1H), 8.36 (d, J = 8.4 Hz, 1H), 8.32 (d, J = 1.6 Hz, 1H), 8.17 (dd, J = 1.6, 8.4 Hz, 1H), 7.41 (s, 1H), 4.75 (s, 2H), 4.14 (s, 2H) |
| 4-48 | 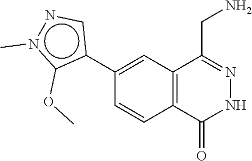 | 4A | 4-2 | 4-(aminomethyl)-6-(5-methoxy-1-methyl-1H- pyrazol-4-yl)phthalazin-1(2H)-one LCMS [M + 1]⁺ = 286.1; ¹H NMR (400 MHz, DMSO-d6) δ = 12.41 (br s, 1H), 8.23 (d, J = 8.4 Hz, 1H), 8.18 (d, J = 1.2 Hz, 1H), 8.03 (dd, J = 1.6, 8.4 Hz, 1H), 8.00 (s, 1H), 4.09-3.97 (s, 2H), 3.90 (s, 3H), 3.73 (s, 3H). |
| 4-49 | 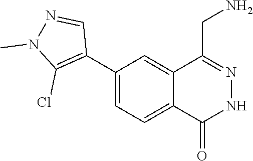 [SMILES: Cn1ncc(-c2ccc3c(=O)[nH]nc(CN)c3c2)c1Cl] | 4A | 4-1 | 4-(aminomethyl)-6-(5-chloro-1-methyl-1H- pyrazol-4-yl)phthalazin-1(2H)-one LCMS [M + 1]⁺ = 290.0; ¹H NMR (400 MHz, DMSO-d6) δ = 12.92 (s, 1H), 8.53 (br s, 3H), 8.34 (d, J = 8.4 Hz, 1H), 8.26 (s, 1H), 8.20 (dd, J = 1.6, 8.4 Hz, 1H), 8.10 (d, J = 1.2 Hz, 1H), 4.52 (br d, J = 5.6 Hz, 2H), 3.91 (s, 3H) |
| 4-50 | 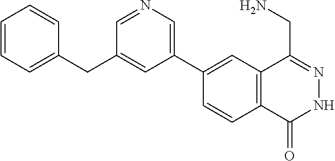 [SMILES: NCc1n[nH]c(=O)c2ccc(-c3cncc(Cc4ccccc4)c3)cc12] | 4A | 4-1 | 4-(aminomethyl)-6-(5-benzylpyridin-3- yl)phthalazin-1(2H)-one LCMS [M + 1]⁺ = 343.1; ¹H NMR (400 MHz, DMSO-d6) δ = 13.01 (s, 1H), 9.05 (d, J = 2.0 Hz, 1H), 8.67 (d, J = 1.6 Hz, 1H), 8.48 (br s, 3H), 8.41- 8.37 (m, 1H), 8.35 (t, J = 2.0 Hz, 1H), 8.29- 8.20 (m, 2H), 7.37-7.27 (m, 4H), 7.23-7.18 (m, 1H), 4.60 (br d, J = 5.6 Hz, 2H), 4.12 (s, 2H) |
| 4-51 | 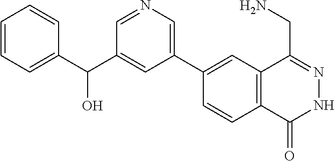 [SMILES: NCc1n[nH]c(=O)c2ccc(-c3cncc(C(O)c4ccccc4)c3)cc12] | 4A | 4-5 | 4-(aminomethyl)-6-(5- (hydroxy(phenyl)methyl)pyridin-3-yl)phthalazin- 1(2H)-one LCMS [M + 1]⁺ = 359.1; ¹H NMR (400 MHz, DMSO-d6) δ = 12.7 (br s, 1H), 8.96 (d, J = 2.4 Hz, 1H), 8.67 (d, J = 2.0 Hz, 1H), 8.36 (d, J = 8.4 (Hz, 1H), 8.29 (d, J = 1.2 Hz, 1H), 8.24 (s, 1H), 8.17 (dd, J = 1.6, 8.4 Hz, 1H), 7.48 (d, J = 7.2 Hz, 2H), 7.38-7.30 (m, 2H), 7.27-7.16 (m, 1H), 6.22 (br d, J = 3.6 Hz, 1H), 5.91 (br s, 1H), 4.27 (br s, 2H) |
| 4-52 | 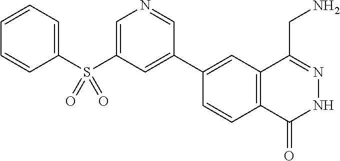 [SMILES: NCc1n[nH]c(=O)c2ccc(-c3cncc(S(=O)(=O)c4ccccc4)c3)cc12] | 4A | 4-6 | 4-(aminomethyl)-6-(5-(phenylsulfonyl)pyridin-3- yl)phthalazin-1(2H)-one LCMS [M + 1]⁺ = 393.1; ¹H NMR (400 MHz, DMSO-d6) δ = 13.02 (s, 1H), 9.43 (d, J = 1.2 Hz, 1H), 9.24 (d, J = 1.2 Hz, 1H), 8.87 (s, 1H), 8.63 (br s, 3H), 8.42-8.32 (m, 3H), 8.15 (br d, J = 7.6 Hz, 2H), 7.74 (br d, J = 7.2 Hz, 1H), 7.70-7.64 (m, 2H), 4.60 (br d, J = 5.2 Hz, 2H). |
| 4-53 | 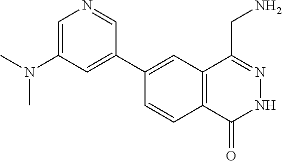 [SMILES: CN(C)c1cncc(-c2ccc3c(=O)[nH]nc(CN)c3c2)c1] | 4A | 4-1 | 4-(aminomethyl)-6-(5-(dimethylamino)pyridin-3- yl)phthalazin-1(2H)-one LCMS [M + 1]⁺ = 296.0; ¹H NMR (400 MHz, DMSO-d6) δ = 13.06 (s, 1H), 8.70 (s, 1H), 8.68 (s, 3H), 8.46-8.35 (m, 3H), 8.26 (d, J = 2.4 Hz, 1H), 8.09 (s, 1H), 4.60 (br d, J = 5.6 Hz, 2H), 3.17 (s, 6H). |
| 4-54 | 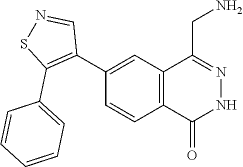 [SMILES: NCc1n[nH]c(=O)c2ccc(-c3cnsc3-c3ccccc3)cc12] | 4A | 4-6 | 4-(aminomethyl)-6-(5-phenylisothiazol-4- yl)phthalazin-1(2H)-one LCMS [M + 1]⁺ = 335.0; ¹H NMR (400 MHz, DMSO-d6) δ = 12.96 (s, 1H), 8.96 (s, 1H), 8.45 (br s, 3H), 8.18 (d, J = 8.2 Hz, 1H), 8.10 (d, J = 1.2 Hz, 1H), 7.65 (dd, J = 1.6, 8.2 Hz, 1H), 7.49- 7.41 (m, 3H), 7.37 (m, 1H), 7.35 (m, 1H), 4.38 (s, 2H) |
| 4-55 | 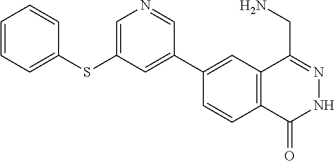 [SMILES: NCc1n[nH]c(=O)c2ccc(-c3cncc(Sc4ccccc4)c3)cc12] | 4A | 4-1 | 4-(aminomethyl)-6-(5-(phenylthio)pyridin-3- yl)phthalazin-1(2H)-one LCMS [M + 1]⁺ = 361.1; ¹H NMR (400 MHz, DMSO-d6) δ = 13.01 (s, 1H), 9.12 (d, J = 2.0 Hz, 1H), 8.61 (br s, 3H), 8.55 (d, J = 2.0 Hz, 1H), 8.46 (t, J = 2.0 Hz, 1H), 8.39-8.35 (m, 1H), 8.32 (d, J = 1.2 Hz, 1H), 8,30-8.26 (m, 1H), 7.49-7.36 (m, 5H), 4.58 (br d, J = 5.6 Hz, 2H). |
| 4-56 | 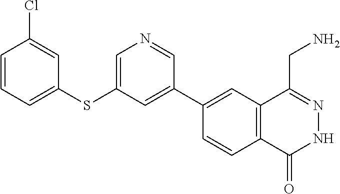 [SMILES: NCc1n[nH]c(=O)c2ccc(-c3cncc(Sc4cccc(Cl)c4)c3)cc12] | 4A | 4-4 | 4-(aminomethyl)-6-(5-((3- chlorophenyl)thio)pyridin-3-yl)phthalazin-1(2H)- one LCMS [M + 1]⁺ = 395.0; ¹H NMR (400 MHz, DMSO-d6) δ = 13.01 (s, 1H), 9.15 (d, J = 2.0 Hz, 1H), 8.69 (d, J = 2.4 Hz, 1H), 8.50 (t, J = 2.0 Hz, 1H), 8.49-8.41 (m, 3H), 8.40-8.37 (m, 1H), 8.33-8.30 (m, 2H), 7.45-7.43 (m, 1H), 7.43- 7.36 (m, 2H), 7.36-7.32 (m, 1H), 4.60 (br d, J = 5.6 Hz, 2H). |
| 4-57 | 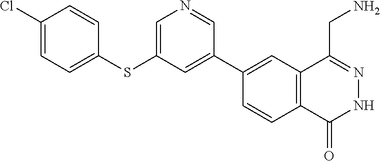 [SMILES: NCc1n[nH]c(=O)c2ccc(-c3cncc(Sc4ccc(Cl)cc4)c3)cc12] | 4A | 4-4 | 4-(aminomethyl)-6-(5-((4- chlorophenyl)thio)pyridin-3-yl)phthalazin-1(2H)- one LCMS [M + 1]⁺ = 395.0; ¹H NMR (400 MHz, DMSO-d6) δ = 13.01 (s, 1H), 9.11 (d, J = 2.0 Hz, 1H), 8.62 (d, J = 2.4 Hz, 1H), 8.44 (t, J = 2.0 Hz, 1H), 8.43-8.33 (m, 4H), 8.31-8.28 (m, 2H), 7.50-7.46 (m, 2H), 7.45-7.42 (m, 2H), 4.60 (d, J = 5.2 Hz, 2H). |
| 4-58 | 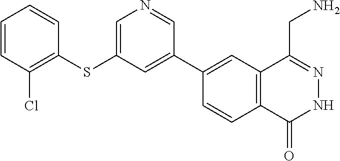 [SMILES: NCc1n[nH]c(=O)c2ccc(-c3cncc(Sc4ccccc4Cl)c3)cc12] | 4A | 4-5 | 4-(aminomethyl)-6-(5-((2- chlorophenyl)thio)pyridin-3-yl)phthalazin-1(2H)- one LCMS [M + 1]⁺ = 395.1; ¹H NMR (400 MHz, DMSO-d6) δ = 13.06-12.98 (s, 1H), 9.19 (d, J = 2.4 Hz, 1H), 8.64 (d, J = 2.0 Hz, 1H), 8.52 (t, J = 2.0 Hz, 1H), 8.49-8.38 (s, 3H), 8.38-8.35 (m, 1H), 8.34-8.30 (m, 2H), 7.65-7.57 (m, 1H), 7.40-7.30 (m, 2H), 7.20-7.12 (m, 1H), 4.61 (d, J = 5.2 Hz, 2H) |
| 4-59 | 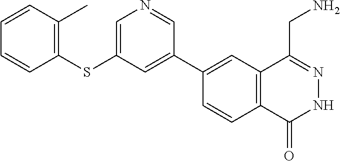 [SMILES: Cc1ccccc1Sc1cncc(-c2ccc3c(=O)[nH]nc(CN)c3c2)c1] | 4A | 4-4 | 4-(aminomethyl)-6-(5-(o-tolylthio)pyridin-3- yl)phthalazin-1(2H)-one LCMS [M + 1]⁺ = 375.1; ¹H NMR (400 MHz, DMSO-d6) δ = 13.00 (s, 1H), 9.03 (d, J = 2.0 Hz, 1H), 8.51-8.38 (m, 3H), 8.38-8.31 (m, 2H), 8.27 (br s, 1H), 8.27-8.26 (m, 1H), 8.24 (br d, J = 1.6 Hz, 1H), 7.42-7.37 (m, 1H), 7.36-7.31 (m, 2H), 7.29-7.24 (m, 1H), 4.59 (br d, J = 5.2 Hz, 2H), 2.40 (s, 3H) |
| 4-60 | 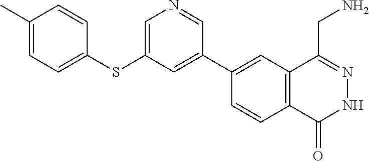 [SMILES: Cc1ccc(Sc2cncc(-c3ccc4c(=O)[nH]nc(CN)c4c3)c2)cc1] | 4A | 4-5 | 4-(aminomethyl)-6-(5-(p-tolylthio)pyridin-3- yl)phthalazin-1(2H)-one LCMS [M + 1]⁺ = 375.1; ¹H NMR (400 MHz, DMSO-d6) δ = 8.91 (d, J = 2.0 Hz, 1H), 8.50 (d, J = 2.0 Hz, 1H), 8.35 (d, J = 8.8 Hz, 1H), 8.31 (s. 1H), 8.13-8.09 (m, 2H), 7.43 (d, J = 8.0 Hz, 2H), 7.27 (d, J = 8.0 Hz, 2H), 4.15-4.09 (m, 2H), 2.35 (s, 3H) |
| 4-61 | 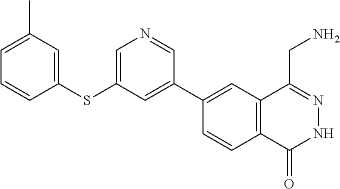 [SMILES: Cc1cccc(Sc2cncc(-c3ccc4c(=O)[nH]nc(CN)c4c3)c2)c1] | 4A | 4-2 | 4-(aminomethyl)-6-(5-(m-tolylthio)pyridin-3- yl)phthalazin-1(2H)-one LCMS [M + 1]⁺ = 375.1; ¹H NMR (400 MHz, DMSO-d6) δ = 8.95 (s, 1H), 8.55 (d, J = 1.6 Hz, 1H), 8.36 (d, J = 8.4 Hz, 1H), 8.33 (s, 1H), 8.19 (s, 1H), 8.13 (br d, J = 8.4 Hz, 1H), 7.35 (s, 1H), 7.32 (d, J = 7.2 Hz, 1H), 7.30-7.26 (m, 1H), 7.21 (br d, J = 7.2 Hz, 1H), 4.19-4.05 (m, 2H), 2.33 (s, 3H) |
| 4-62 | 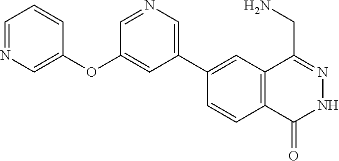 [SMILES: NCc1n[nH]c(=O)c2ccc(-c3cncc(Oc4cccnc4)c3)cc12] | 4A | 4-2 | 4-(aminomethyl)-6-(5-(pyridin-3-yloxy)pyridin-3- yl)phthalazin-1(2H)-one LCMS [M + 1]⁺ = 346.1; ¹H NMR (400 MHz, DMSO-d6) δ = 8.91 (d, J = 1.6 Hz, 1H), 8.51 (d, J = 2.8 Hz, 1H), 8.49 (d, J = 2.4 Hz, 1H), 8.43 (dd, J = 1.2, 4.8 Hz, 1H), 8.38-8.33 (m, 2H), 8.16 (dd, J = 1.6, 8.4 Hz, 1H), 8.02 (t, J = 2.4 Hz, 1H), 7.59 (ddd, J = 1.2, 2.8, 8.4 Hz, 1H), 7.46 (dd, J = 4.4, 8.4 Hz, 1H), 4.12 (s, 2H) |
| 4-63 | 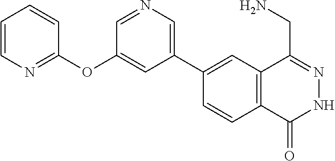 [SMILES: NCc1n[nH]c(=O)c2ccc(-c3cncc(Oc4ccccn4)c3)cc12] | 4A | 4-5 | 4-(aminomethyl)-6-(5-(pyridin-2-yloxy)pyridin-3- yl)phthalazin-1(2H)-one LCMS [M + 1]⁺ = 346.1; ¹H NMR (400 MHz, DMSO-d6) δ = 13.00 (s, 1H), 9.03 (d, J = 1.6 Hz, 1H), 8.59 (d, J = 2.4 Hz, 1H), 8.38 (br d, J = 8.0 Hz, 4H), 8.35-8.29 (m, 2H), 8.26 (t, J = 2.0 Hz, 1H), 8.16 (dd, J = 1.2, 4.9 Hz, 1H), 7.94 (td, J = 2.0, 7.2, 8.4 Hz, 1H), 7.24-7.16 (m, 2H), 4.64- 4.56 (d, J = 5.6 Hz, 2H). |
| 4-64 | 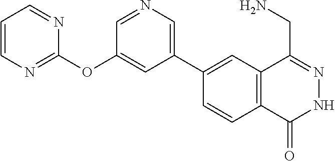 [SMILES: NCc1n[nH]c(=O)c2ccc(-c3cncc(Oc4ncccn4)c3)cc12] | 4B | 4-2 | 4-(aminomethyl)-6-(5-(pyrimidin-2- yloxy)pyridin-3-yl)phthalazin-1(2H)-one LCMS [M + 1]⁺ = 347.2; ¹H NMR (400 MHz, DMSO-d6) δ = 12.55 (br s, 1H), 9.02 (d, J = 2.0 Hz, 1H), 8,70 (d, J = 4.8 Hz, 2H), 8,64 (d, J = 2.4 Hz, 1H), 8.40 (d, J = 1.6 Hz, 1H), 8.36-8.32 (m, 2H), 8.26-8.22 (m, 1H), 7.37-7.32 (m, 1H), 4.09 (s, 2H). |
| 4-65 | 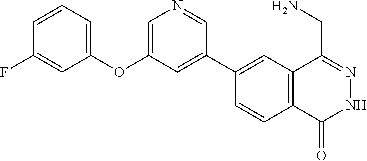 [SMILES: NCc1n[nH]c(=O)c2ccc(-c3cncc(Oc4cccc(F)c4)c3)cc12] | 4A | 4-1 | 4-(aminomethyl)-6-(5-(3-fluorophenoxy)pyridin- 3-yl)phthalazin-1(2H)-one LCMS [M + 1]⁺ = 363.1; ¹H NMR (400 MHz, DMSO-d6) δ = 13.00 (s, 1H), 9.09 (d, J = 2.0 Hz, 1H), 8.65 (br s, 3H), 8.55 (d, J = 2.8 Hz, 1H), 8.39- 8.34 (m, 2H), 8.33-8.27 (m, 2H), 7.51-7.43 (m, 1H), 7.10-7.02 (m, 2H), 7.00-6.96 (d, J = 7.2 Hz, 1H), 4.61-4.54 (d, J = 6.0 Hz, 2H). |
| 4-66 | 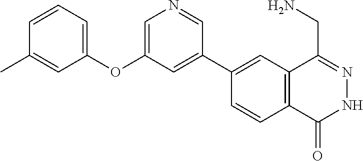 [SMILES: Cc1cccc(Oc2cncc(-c3ccc4c(=O)[nH]nc(CN)c4c3)c2)c1] | 4A | 4-1 | 4-(aminomethyl)-6-(5-(m-tolyloxy)pyridin-3- yl)phthalazin-1(2H)-one LCMS [M + 1]⁺ 359.1; ¹H NMR (400 MHz, DMSO-d6) δ = 12.76 (s, 1H), 8.93 (d, J = 2.0 Hz, 1H), 8.54-8.31 (m, 5H), 8.31-8.26 (m, 1H), 8.24 (d, J = 8.4 Hz, 1H), 8.01 (t, J = 2.0 Hz, 1H), 7.32 (t, J = 8.0 Hz, 1H), 7.03 (d, J = 7.6 Hz, 1H), 6.97 (s, 1H), 6.93 (dd, J = 2.8, 8.0 Hz, 1H), 4.55 (s, 2H), 2.34 (s, 3H) |
| 4-67 | 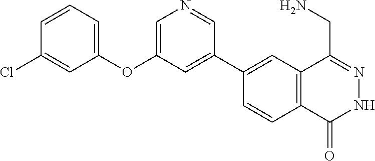 [SMILES: NCc1n[nH]c(=O)c2ccc(-c3cncc(Oc4cccc(Cl)c4)c3)cc12] | 4A | 4-1 | 4-(aminomethyl)-6-(5-(3-chlorophenoxy)pyridin- 3-yl)phthalazin-1(2H)-one LCMS [M + 1]⁺ = 379.1; ¹H NMR (400 MHz, DMSO-d6) δ = 13.00 (s, 1H), 9.03 (d, J = 2.0 Hz, 1H), 8.52 (d, J = 2.8 Hz, 1H), 8.44 (br s, 3H), 8.40- 8.36 (d, J = 8.8 Hz, 1H), 8.33-8.28 (m, 2H), 8.18 (t, J = 2.4 Hz, 1H), 7.48-7.43 (t, J = 8.0 Hz, 1H), 7.28-7.24 (m, 1H), 7.22 (t, J = 2.0 Hz, 1H), 7.10 (td, J = 0.8, 8.4 Hz, 1H), 4.60 (br d, J = 5.6 Hz, 2H) |
| 4-68 | 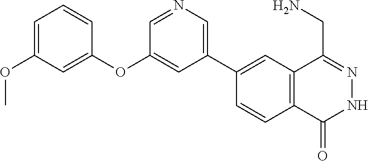 [SMILES: COc1cccc(Oc2cncc(-c3ccc4c(=O)[nH]nc(CN)c4c3)c2)c1] | 4A | 4-1 | 4-(aminomethyl)-6-(5-(3- methoxyphenoxy)pyridin-3-yl)phthalazin-1(2H)- one LCMS [M + 1]⁺ 375.1; ¹H NMR (400 MHz, DMSO-d6) δ = 12.76 (s, 1H), 8.94 (d, J = 2.0 Hz, 1H), 8.48 (br s, 3H), 8.45 (d, J = 2.4 Hz, 1H), 8.40 (d, J = 8.0 Hz, 1H), 8.29 (d, J = 1.6 Hz, 1H), 8.24 (dd, J = 1.6, 8.4 Hz, 1H), 8.04 (t, J = 2.4 Hz, 1H), 7.34 (t, J = 8.0 Hz, 1H), 6.80 (dd, J = 2.0, 8.0 Hz, 1H), 6.72 (t, J = 2.4 Hz, 1H), 6.71-6.67 (dd, J = 2.0, 6.8 Hz, 1H), 4.55 (s, 2H), 3.79 (s, 3H) |
| 4-69 | 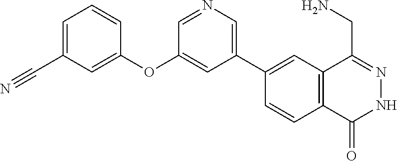 [SMILES: N#Cc1cccc(Oc2cncc(-c3ccc4c(=O)[nH]nc(CN)c4c3)c2)c1] | 4A | 4-1 | 3-((5-(4-(aminomethyl)-1-oxo-1,2- dihydrophthalazin-6-yl)pyridin-3- yl)oxy)benzonitrile LCMS [M + 1]⁺ = 370.1; ¹H NMR (400 MHz, DMSO-d6) δ = 13.00 (s, 1H), 9.12 (d, J = 2.0 Hz, 1H), 8.65 (br s, 3H), 8.60 (d, J = 2.8 Hz, 1H), 8.39- 8.30 (m, 4H), 7.70-7.62 (m, 3H), 7.54-7.49 (m, 1H), 4.57 (br d, J = 5.6 Hz, 2H). |
| 4-70 | 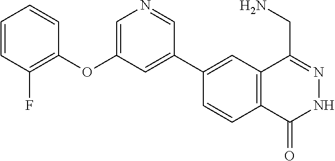 [SMILES: NCc1n[nH]c(=O)c2ccc(-c3cncc(Oc4ccccc4F)c3)cc12] | 4A | 4-5 | 4-(aminomethyl)-6-(5-(2-fluorophenoxy)pyridin- 3-yl)phthalazin-1(2H)-one LCMS [M + 1]⁺ = 363.0; ¹H NMR (400 MHz, DMSO-d6) δ = 13.01 (s, 1H), 8.97 (d, J = 1.6 Hz, 1H), 8.43 (br d, J = 2.4 Hz, 3H), 8.42-8.35 (m, 2H), 8.32-8.26 (m, 2H), 8.11-8.05 (m, 1H), 7.50-7.42 (m, 1H), 7.35-7.24 (m, 3H), 4.61 (br d, J = 5.6 Hz, 2H) |
| 4-71 | 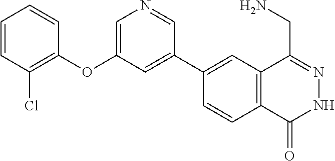 [SMILES: NCc1n[nH]c(=O)c2ccc(-c3cncc(Oc4ccccc4Cl)c3)cc12] | 4A | 4-1 | 4-(aminomethyl)-6-(5-(2-chlorophenoxy)pyridin- 3-yl)phthalazin-1(2H)-one LCMS [M + 1]⁺ = 379.1; ¹H NMR (400 MHz, DMSO-d6) δ = 13.00 (s, 1H), 9.07 (d, J = 2.0 Hz, 1H), 8.67 (br s, 3H), 8.42 (d, J = 2.8 Hz, 1H), 8.39- 8.35 (m, 2H), 8.31-8.27 (m, 1H), 8.25 (t, J = 2.0 Hz, 1H), 7.67 (dd, J = 1.2, 8.0 Hz, 1H), 7.42 (dd, J = 1.6, 7.8 Hz, 1H), 7.34-7.26 (m, 2H), 4.58 (br d, J = 5.6 Hz, 2H). |
| 4-72 | 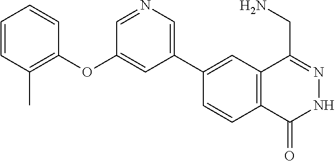 [SMILES: Cc1ccccc1Oc1cncc(-c2ccc3c(=O)[nH]nc(CN)c3c2)c1] | 4A | 4-1 | 4-(aminomethyl)-6-(5-(o-tolyloxy)pyridin-3- yl)phthalazin-1(2H)-one LCMS [M + 1]⁺ = 359.1; ¹H NMR (400 MHz, DMSO-d6) δ = 13.00 (s, 1H), 8.96 (d, J = 2.0 Hz, 1H), 8.54 (br s, 3H), 8.37 (d, J = 8.4 Hz, 1H), 8.33- 8.30 (m, 2H), 8.26 (dd, J = 1.6, 8.4 Hz, 1H), 8.05 (t, J = 2.4 Hz, 1H), 7.39 (d, J = 7.6 Hz, 1H), 7.31- 7.23 (td, J = 0.8, 7.2 Hz, 1H), 7.20-7.14 (td, J = 0.8, 7.2 Hz, 1H), 7.02 (d, J = 7.6 Hz, 1H), 4.59 (br d, J = 5.6 Hz, 2H), 2.27 (s, 3H) |
| 4-73 | 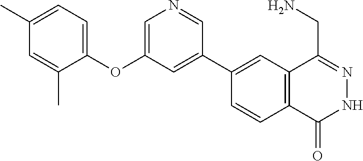 [SMILES: Cc1ccc(Oc2cncc(-c3ccc4c(=O)[nH]nc(CN)c4c3)c2)c(C)c1] | 4A | 4-6 | 4-(aminomethyl)-6-(5-(2,4- dimethylphenoxy)pyridin-3-yl)phthalazin-1(2H)- one LCMS [M + 1]⁺ = 373.1; ¹H NMR (400 MHz, DMSO-d6) δ = 13.00 (s, 1H), 8.94 (d, J = 1.8 Hz, 1H), 8.56 (br s, 3H), 8.38 (d, J = 8.4 Hz, 1H), 8.32 (d, J = 1.2 Hz, 1H), 8.29 (d, J = 2.4 Hz, 1H), 8.25 (dd, J = 1.6, 8.4 Hz, 1H), 8.01 (t, J = 2.2 Hz, 1H), 7.20 (s, 1H), 7.08 (dd, J = 2.0, 8.0 Hz, 1H), 6.95 (d, J = 8.0 Hz, 1H), 4.59 (br d, J = 5.6 Hz, 2H), 2.31 (s, 3H), 2.21 (s, 3H) |
| 4-74 | 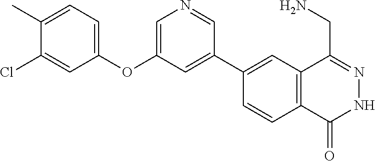 [SMILES: Cc1ccc(Oc2cncc(-c3ccc4c(=O)[nH]nc(CN)c4c3)c2)cc1Cl] | 4A | 4-2 | 4-(aminomethyl)-6-(5-(3-chloro-4- methylphenoxy)pyridin-3-yl)phtbalazin-1(2H)- one LCMS [M + 1]⁺ = 393.0; ¹H NMR (400 MHz, DMSO-d6) δ = 12.60-12.48 (s, 1H), 8.93 (d, J = 2.0 Hz, 1H), 8.46 (d, J = 2.4 Hz, 1H), 8.38 (d, J = 1.6 Hz, 1H), 8.34 (d, J = 8.4 Hz, 1H), 8.20 (dd, J = 1.6, 8.4 Hz, 1H), 8.07 (t, J = 2.4 Hz, 1H), 7.41 (d, J = 8.4 Hz, 1H), 7.26 (d, J = 2.4 Hz, 1H), 7.06 (dd, J = 2.4, 8.4 Hz, 1H), 4,09 (s, 2H), 2.33 (s, 3H) |
| 4-75 | 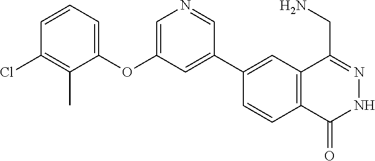 [SMILES: Cc1c(Cl)cccc1Oc1cncc(-c2ccc3c(=O)[nH]nc(CN)c3c2)c1] | 4A | 4-2 | 4-(aminomethyl)-6-(5-(3-chloro-2- methylphenoxy)pyridin-3-yl)phthalazin-1(2H)- one LCMS [M + 1]⁺ = 393.0; ¹H NMR (400 MHz, DMSO-d6) δ = 12.55 (br s, 1H), 8.91 (d, J = 2.0 Hz, 1H), 8.38 (d, J = 2.4 Hz, 2H), 8.34 (d, J = 8.4 Hz, 1H), 8.18 (dd, J = 1.6, 8.4 Hz, 1H), 7.99 (t, J = 2.4 Hz, 1H), 7.36-7.32 (dd, J = 1.6, 8.4 Hz, 1H), 7.31-7.25 (t, J = 8.0 Hz, 1H), 7.01 (d, J = 7.6 Hz, 1H), 4.09 (s, 2H), 2.34 (s, 3H) |
| 4-76 | 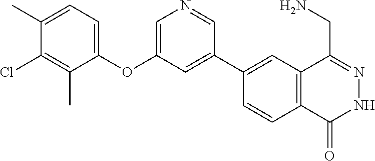 [SMILES: Cc1ccc(Oc2cncc(-c3ccc4c(=O)[nH]nc(CN)c4c3)c2)c(C)c1Cl] | 4A | 4-2 | 4-(aminomethyl)-6-(5-(3-chloro-2.4- dimethylphenoxy)pyridin-3-yl)phthalazin-1(2H)- one LCMS [M + 1]⁺ = 407.0; ¹H NMR (400 MHz, DMSO-d6) δ = 12.54 (br s, 1H), 8.85 (d, J = 1.6 Hz, 1H), 8.34 (s, 1H), 8.31 (m, 2H), 8.14 (br d, J = 8.4 Hz, 1H), 7.90 (s, 1H), 7.25 (br d, J = 8.4 Hz, 1H), 6.96 (d, J = 8.4 Hz, 1H), 4.08 (s, 2H), 2.33 (s, 3H), 2.30 (s, 3H) |
| 4-77 | 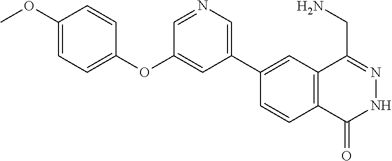 [SMILES: COc1ccc(Oc2cncc(-c3ccc4c(=O)[nH]nc(CN)c4c3)c2)cc1] | 4A | 4-1 | 4-(aminomethyl)-6-(5-(4- methoxyphenoxy)pyridin-3-yl)phthalazin-1(2H)- one LCMS [M + 1]⁺ = 375.1; ¹H NMR (400 MHz, DMSO-d6) δ = 13.00 (s, 1H), 8.95 (d, J = 2.0 Hz, 1H), 8.55 (br s, 3H), 8.39-8.36 (m, 2H), 8.31 (d, J = 1.2 Hz, 1H), 8.26 (dd, J = 1.6, 8.4 Hz, 1H), 8.05 (t, J = 2.2 Hz, 1H), 7.17-7.14 (m, 2H), 7.04- 7.00 (m, 2H), 4.58 (br d, J = 5.6 Hz, 2H), 3.77 (s, 3H) |
| 4-78 | 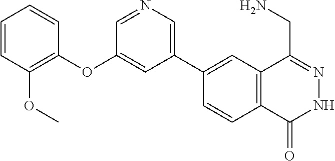 [SMILES: COc1ccccc1Oc1cncc(-c2ccc3c(=O)[nH]nc(CN)c3c2)c1] | 4A | 4-1 | 4-(aminomethyl)-6-(5-(2- methoxyphenoxy)pyridin-3-yl)phthalazin-1(2H)- one LCMS [M + 1]⁺ = 375.1; ¹H NMR (400 MHz, DMSO-d6) δ = 12.99 (s, 1H), 8.89 (d, J = 1.6 Hz, 1H), 8.53 (br s, 3H), 8.37 (d, J = 8.4 Hz, 1H), 8,29 (d, J = 1.2 Hz, 1H), 8.25-8.21 (m, 2H), 7.96- 7.93 (t, J = 2.0 Hz, 1H), 7.32-7.27 (dt, J = 1.6, 7.6 Hz, 1H), 7.26-7.22 (dd, J = 1.6, 8,4, 1H), 7.21 (dd, J = 1.6, 7.6 Hz, 1H), 7.04 (dt, J = 1.6, 7.6 Hz, 1H), 4.59 (br d, J = 5.6 Hz, 2H), 3.78 (s, 3H) |
| 4-79 | 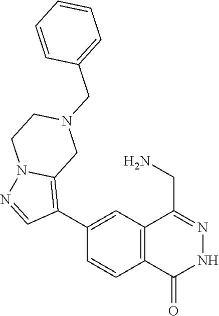 [SMILES: NCc1n[nH]c(=O)c2ccc(-c3cnn4c3CN(Cc3ccccc3)CC4)cc12] | 4B | 4-2 | 4-(aminomethyl)-6-(5-benzyl-4,5,6,7- tetrahydropyrazolo[1,5-a]pyrazin-3-yl)phthalazin- 1(2H)-one LCMS [M + 1]⁺ 387.1; ¹H NMR (400 MHz, DMSO-d6) δ = 12.40 (br s, 1H), 8.20 (d, J = 8.0 Hz, 1H), 8.05 (s, 1H), 7.88 (s, 1H), 7.85 (dd, J = 1.6, 8.4 Hz, 1H), 7.42-7.31 (m, 4H), 7.30-7.23 (m, 1H), 4.18 (br t, J = 5.4 Hz, 2H), 3.99 (s, 2H), 3.95 (s, 2H), 3.81 (s, 2H), 2.98 (br t, J = 5.4 Hz, 2H) |
| 4-80 | 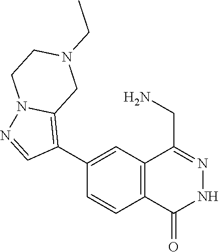 [SMILES: CCN1CCn2ncc(-c3ccc4c(=O)[nH]nc(CN)c4c3)c2C1] | 4B | 4-4 | 4-(aminomethyl)-6-(5-ethyl-4,5,6,7- tetrahydropyrazolo[1,5-a]pyrazin-3-yl)phthalazin- 1(2H)-one LCMS [M + 1]⁺: 325.3; ¹H NMR (400 MHz, MeOD) δ = 8.44 (d, J = 8.4 Hz, 1H), 8.14 (s, 1H), 7.97 (dd, J = 1.6, 8.4 Hz, 1H), 7.90 (s, 1H), 5.02 (s, 2H), 4.71 (s, 2H), 4.64 (br t, J = 5.6 Hz, 2H), 4.01 (br s, 2H), 3.58 (q, J = 7.2 Hz, 2H), 1.51 (t, J = 7.2 Hz, 3H). |
| 4-81 | 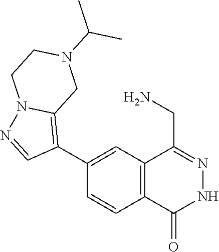 [SMILES: CC(C)N1CCn2ncc(-c3ccc4c(=O)[nH]nc(CN)c4c3)c2C1] | 4C | 4-2 | 4-(aminomethyl)-6-(5-isopropyl-4,5,6,7- tetrahydropyrazolo[1,5-a]pyrazin-3-yl)phthalazin- 1(2H)-one LCMS [M + 1]⁺ = 339.1; ¹H NMR (400 MHz, DMSO-d6) δ = 8.25 (d, J = 8.2 Hz, 1H), 7.98 (d, J = 1.6 Hz, 1H), 7.95 (s, 1H), 7.87 (dd, J = 1.6, 8.4 Hz, 1H), 4.16 (t, J = 5.6 Hz, 2H), 4.08 (s, 2H), 4.02 (s, 2H), 3.03 (t, J = 5.6 Hz, 2H), 2.55 (td, J = 2.0, 4.0 Hz, 1H), 1.13 (d, J = 6.8 Hz, 6 H) |
| 4-82 | 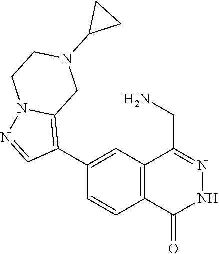 [SMILES: NCc1n[nH]c(=O)c2ccc(-c3cnn4c3CN(C3CC3)CC4)cc12] | 4B | 4-2 | 4-(aminomethyl)-6-(5-cyclopropyl-4,5,6,7- tetrahydropyrazolo[1,5-a]pyrazin-3-yl)phthalazin- 1(2H)-one LCMS [M + 1]⁺ 337.3; ¹H NMR (400 MHz, MeOD) δ = 8.36 (d, J = 8.4 Hz, 1H), 7.99 (s, 1H), 7.87 (m, 2H), 4.23 (br s, 2H), 4.19 (s, 2H), 4.14 (s, 2H), 3.24 (br s, 2H), 2.07 (br s, 1H), 0.62 (br d, J = 5.2 Hz, 2H), 0.54 (br s, 2H). |
| 4-83 | 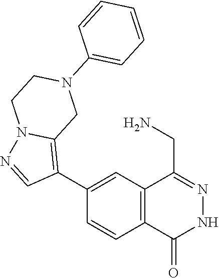 [SMILES: NCc1n[nH]c(=O)c2ccc(-c3cnn4c3CN(c3ccccc3)CC4)cc12] | 4A | 4-1 | 4-(aminomethyl)-6-(5-phenyl-4,5,6,7- tetrahydropyrazolo[1,5-a]pyrazin-3-yl)phthalazin- 1(2H)-one LCMS [M + 1]⁺ = 373.1; ¹H NMR (400 MHz, DMSO-d6) δ = 12.88 (s, 1H), 8.56 (br s, 3H), 8.31 (d, J = 8.4 Hz, 1H), 8.19 (s, 1H), 8.05 (dd, J = 1.2, (8.4 Hz, 1H), 7.93 (d, J = 1.2, 1H), 7.31-7.23 (m, 2H), 7.15 (d, J = 8.0 Hz, 2H), 6.86 (t, J = 7.2 Hz, 1H), 4.82 (s, 2H), 4.58 (br d, J = 5.6 Hz, 2H), 4.29 (br t, J = 5.2 Hz, 2H), 3.85 (br t, J = 5.2 Hz, 2H) |
| 4-84 | 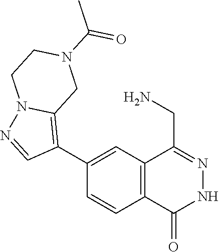 [SMILES: CC(=O)N1CCn2ncc(-c3ccc4c(=O)[nH]nc(CN)c4c3)c2C1] | 4B | 4-1 | 6-(5-acetyl-4,5,6,7-tetrahydropyrazolo[1,5- a]pyrazin-3-yl)-4-(aminomethyl)phthalazin- 1(2H)-one LCMS [M + 1]⁺ 339.1; ¹H NMR (400 MHz, DMSO-d6) δ = 12.64 (br s, 1H), 8.55 (br s, 3H), 8.33 (d, J = 8.0 Hz, 1H), 8.11 (s, 1H), 7.94 (br d, J = 8.4 Hz, 1H), 7.91 (d, J = 1.2 Hz, 1H), 5.05 (br s, 2H), 4.48 (br s, 2H), 4.26 (br s, 2H), 4.01 (t, J = 5.6 Hz, 2H), 2.18 (s, 3H) |
| 4-85 | 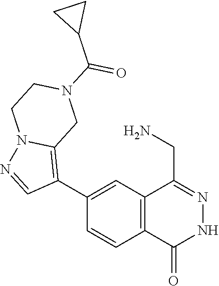 [SMILES: NCc1n[nH]c(=O)c2ccc(-c3cnn4c3CN(C(=O)C3CC3)CC4)cc12] | 4A | 4-5 | 4-(aminomethyl)-6-(5-(cyclopropanecarbonyl)- 4,5,6,7-tetrahydropyrazolo[1,5-a]pyrazin-3- yl)phthalazin-1(2H)-one LCMS [M + 1]⁺ = 365.2; ¹H NMR (400 MHz, DMSO-d6) δ = 8.41 (br s, 3H), 8.33 (d, J = 8.4 Hz, 1H), 8.09 (s, 1H), 7,96 (dd, J = 1.2, 8.4 Hz, 1H), 7.87 (d, J = 1.2 Hz, 1H), 5.14 (s, 2H), 4.51 (s, 2H), 4.33-4.24 (m, 2H), 4.17 (br s, 2H), 2.18- 2.11 (m, 1H), 0.84-0.79 (m, 4H). |
| 4-86 | 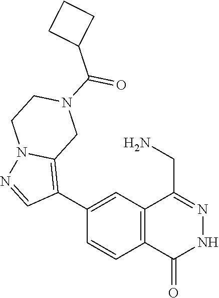 [SMILES: NCc1n[nH]c(=O)c2ccc(-c3cnn4c3CN(C(=O)C3CCC3)CC4)cc12] | 4A | 4-5 | 4-(aminomethyl)-6-(5-(cyclobutanecarbonyl)- 4,5,6,7-tetrahydropyrazolo[1,5-a]pyrazin-3- yl)phthalazin-1(2H)-one LCMS [M + 1]⁺ = 379.1; ¹H NMR (400 MHz, DMSO-d6) δ = 12.2 (s, 1H), 8.29 (d, J = 8.4 Hz, 1H), 8.01 (s, 1H), 8.01 (d, J = 1.6 Hz, 1H), 7.89 (dd, J = 1.6, 8.4 Hz, 1H), 4.97 (s, 2H), 4.21 (t, J = 5.6 Hz, 2H), 4.09 (s, 2H), 3.95 (br s, 2H), 3.53 (t, J = 8.4 Hz, 1H), 2.30-2.18 (m, 4H), 2.00-1.89 (m, 1H), 1.87-1.75 (m, 1H) |
| 4-87 | 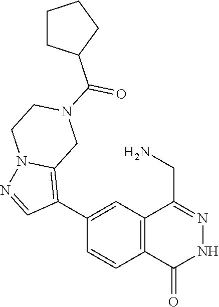 [SMILES: NCc1n[nH]c(=O)c2ccc(-c3cnn4c3CN(C(=O)C3CCCC3)CC4)cc12] | 4A | 4-6 | 4-(aminomethyl)-6-(5-(cyclopentanecarbonyl)- 4,5,6,7-tetrahydropyrazolo[1,5-a]pyrazin-3- yl)phthalazin-1(2H)-one LCMS [M + 1]⁺ = 393.1; ¹H NMR (400 MHz, DMSO-d6) δ = 12.64 (br s, 1H), 8.53 (br s, 3H), 8.33 (d, J = 8.4 Hz, 1H), 8.11 (s, 1H), 7.94 (br d, J = 9.2 Hz, 1H), 7.91 (s, 1H), 5.07 (br s, 2H), 4.48 (s, 2H), 4.24 (br t, J = 5.2 Hz, 2H), 4.07 (br t, J = 5.2 Hz, 2H), 3.23-3.16 (m, 1H), 1.82 (m, 2H), 1.77-1.68 (m, 2H), 1.63-1.62 (m, 2H), 1.60- 1.51 (m, 2H) |
| 4-88 | 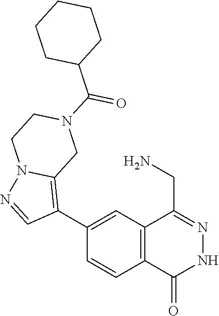 [SMILES: NCc1n[nH]c(=O)c2ccc(-c3cnn4c3CN(C(=O)C3CCCCC3)CC4)cc12] | 4A | 4-4 | 4-(aminomethyl)-6-(5-(cyclohexanecarbonyl)- 4,5,6,7-tetrahydropyrazolo[1,5-a]pyrazin-3- yl)phthalazin-1(2H)-one LCMS [M + 1]⁺ = 407.3; ¹H NMR (400 MHz, DMSO-d6) δ = 12.65 (br s, 1H), 8.52 (br s, 3H), 8.34 (d, J = 8.0 Hz, 1H), 8.12 (s, 1H), 7.95 (d, J = (8.0 Hz, 1H), 7.92 (s, 1H), 5.06 (s, 2H), 4.49 (br s, 2H), 4.28-4.22 (m, 2H), 4.09-4.04 (m, 2H), 2.84-2.75 (m, 1H), 1.73-1.70 (m, 5H), 1.45- 1.23 (m, 5H) |
| 4-89 | 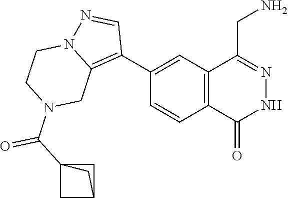 [SMILES: NCc1n[nH]c(=O)c2ccc(-c3cnn4c3CN(C(=O)C35CC(C3)C5)CC4)cc12] | 4A | 4-5 | 4-(aminomethyl)-6-(5-(bicyclo[1.1.1]pentane-1- carbonyl)-4,5,6,7-tetrahydropyrazolo[1,5- a]pyrazin-3-yl)phthalazin-1(2H)-one LCMS [M + 1]⁺ = 391.3; ¹H NMR (400 MHz, DMSO-d6) δ = 12.88 (s, 1H), 8.45 (br s, 3H), 8.32 (br d, J = 8.4 Hz, 1H), 8.16 (s, 1H), 8.00-7.88 (m, 2H), 5.24-4.95 (m, 2H), 4.52 (br s, 2H), 4.32- 4.09 (m, 4H), 2.22-2.06 (m, 7H). |
| 4-90 | 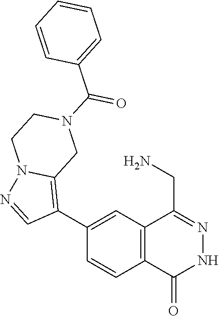 [SMILES: NCc1n[nH]c(=O)c2ccc(-c3cnn4c3CN(C(=O)c3ccccc3)CC4)cc12] | 4A | 4-5 | 4-(aminomethyl)-6-(5-benzoyl-4,5,6,7- tetrahydropyrazolo[1,5-a]pyrazin-3-yl)phthalazin- 1(2H)-one LCMS [M + 1]⁺ = 401.0 ¹H NMR (400 MHz, DMSO-d6) δ = 12.90 (br s, 1H), 8.59-8.37 (s, 3H), 8.36-8.25 (m, 1H), 8.18 (s, 1H), 8.04-7.79 (m, 2H), 7.52 (br d, J = 4.0 Hz, 5H), 5.15 (br s, 2H), 4.52 (br s, 2H), 4.29 (br d, J = 4.8 Hz, 2H), 4.08-3.70 (m, 2H) |
| 4-91 | 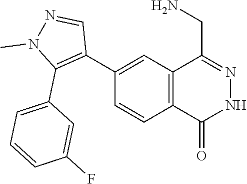 [SMILES: Cn1ncc(-c2ccc3c(=O)[nH]nc(CN)c3c2)c1-c1cccc(F)c1] | 4A | 4-5 | 4-(aminomethyl)-6-(5-(3-fluorophenyl)-1-methyl- 1H-pyrazol-4-yl)phthalazin-1(2H)-one LCMS [M + 1]⁺ = 350.0 ¹H NMR (400 MHz, DMSO-d6) δ = 12.84 (s, 1H), 8.40 (br s, 3H), 8.14 (s, 1H), 8.09 (d, J = 8.4 Hz, 1H), 7.74 (d, J = 1.2 Hz, 1H), 7.64-7.54 (m, 1H), 7.51 (dd, J = 1.6, 8.4 Hz, 1H), 7.44-7.32 (m, 2H), 7.24 (d, J = 7.6 Hz, 1H), 4.27 (s, 2H), 3.76 (s, 3H) |
| 4-92 | 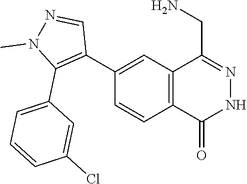 [SMILES: Cn1ncc(-c2ccc3c(=O)[nH]nc(CN)c3c2)c1-c1cccc(Cl)c1] | 4A | 4-5 | 4-(aminomethyl)-6-(5-(3-chlorophenyl)-1-methyl- 1H-pyrazol-4-yl)phthalazin-1(2H)-one LCMS [M + 1]⁺ = 366.1; ¹H NMR (400 MHz, DMSO-d6) δ = 12.84 (s, 1H), 8.46 (br s, 3H), 8.16 (s, 1H), 8.09 (d, J = 8.4 Hz, 1H), 7.79 (d, J = 1.2 Hz, 1H), 7.65-7.61 (m, 1H), 7.60-7.55 (m, 2H), 7.47 (dd, J = 1.6, 8.4 Hz, 1H), 7.40 (td, J = 1.2, 7.6 Hz, 1H), 4.32-4.27 (d, J = 5.6 Hz, 2H), 3.76 (s, 3H) |
| 4-93 | 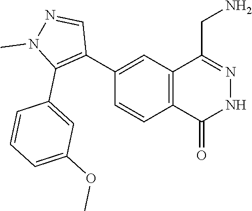 [SMILES: COc1cccc(-c2c(-c3ccc4c(=O)[nH]nc(CN)c4c3)cnn2C)c1] | 4A | 4-4 | 4-(aminomethyl)-6-(5-(3-methoxyphenyl)-1- methyl-1H-pyrazol-4-yl)phthalazin-1(2H)-one LCMS [M + 1]⁺ = 362.1; ¹H NMR (400 MHz, DMSO-d6) δ = 12.83 (s, 1H), 8.39-8.29 (s, 3H), 8.14 (s, 1H), 8.08 (d, J = 8.4 Hz, 1H), 7.75 (d, J = 1.2 Hz, 1H), 7.54 (dd, J = 1.6, 8.4 Hz, 1H), 7.47 (t, J = 8.0 Hz, 1H), 7.15-7.10 (dd, J = 2.8, 8.4 Hz, 1H), 7.01-6.99 (t, J = 1.2 Hz, 1H), 6.98-6.94 (dt, J = 7.6 Hz, 1H), 4.27 (br d, J = 4.8 Hz, 2H), 3.77 (s, 3H), 3.75 (s, 3H). |
| 4-94 | 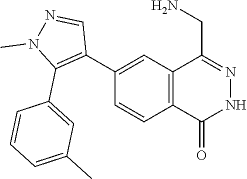 [SMILES: Cc1cccc(-c2c(-c3ccc4c(=O)[nH]nc(CN)c4c3)cnn2C)c1] | 4A | 4-4 | 4-(aminomethyl)-6-(1-methyl-5-(m-tolyl)-1H- pyrazol-4-yl)phthalazin-1(2H)-one LCMS [M + 1]⁺ = 346.1; ¹H NMR (400 MHz, DMSO-d6) δ = 12.83 (s, 1H), 8.35 (br s, 3H), 8.15 (s, 1H), 8.06 (d, J = 8.4 Hz, 1H), 7.72 (d, J = 1.2 Hz, 1H), 7.53 (dd, J = 1.2, 8.4 Hz, 1H), 7.44 (t, J = 7.6 Hz,, 1H), 7.38 (d, J = 7.6 Hz, 1H), 7.26 (s, 1H), 7.21 (d, J = 7.2 Hz, 1H), 4.24 (br s, 2H), 3.73 (s, 3H), 2.36 (s, 3H) |
| 4-95 | 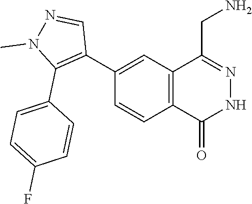 [SMILES: Cn1ncc(-c2ccc3c(=O)[nH]nc(CN)c3c2)c1-c1ccc(F)cc1] | 4A | 4-1 | 4-(aminomethyl)-6-(5-(4-fluorophenyl)-1-methyl- 1H-pyrazol-4-yl)phthalazin-1(2H)-one LCMS [M + 1]⁺ = 350.1; ¹H NMR (400 MHz, DMSO-d6) δ = 12.84 (s, 1H), 8.49 (br s, 3H), 8.16 (s, 1H), 8.08 (d, J = 8.4 Hz, 1H), 7.78 (d, J = 1.2 Hz, 1H), 7.53-7.46 (m, 3H), 7.43-7.37 (m, 2H), 4.28 (br d, J = 5.6 Hz, 2H), 3.74 (s, 3H) |
| 4-96 | 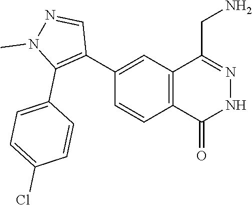 [SMILES: Cn1ncc(-c2ccc3c(=O)[nH]nc(CN)c3c2)c1-c1ccc(Cl)cc1] | 4A | 4-1 | 4-(aminomethyl)-6-(5-(4-chlorophenyl)-1-methyl- 1H-pyrazol-4-yl)phthalazin-1(2H)-one LCMS [M + 1]⁺ = 366.0; ¹H NMR (400 MHz, DMSO-d6) δ = 12.84 (s, 1H), 8.54 (br s, 3H), 8.16 (s, 1H), 8.08 (d, J = 8.4 Hz, 1H), 7.85 (d, J = 1.2 Hz, 1H), 7.63-7.58 (m, 2H), 7.48-7.45 (dd, J = 1.2, 8.4 Hz, 2H), 7.43 (dd, J = 1.2, 8.4 Hz, 1H), 4.33 (br d, J = 5.6 Hz, 2H), 3.75 (s, 3H) |
| 4-97 | 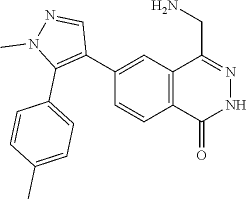 [SMILES: Cc1ccc(-c2c(-c3ccc4c(=O)[nH]nc(CN)c4c3)cnn2C)cc1] | 4A | 4-4 | 4-(aminomethyl)-6-(1-methyl-5-(p-tolyl)-1H- pyrazol-4-yl)phthalazin-1(2H)-one LCMS [M + 1]⁺ = 346.1; ¹H NMR (400 MHz, DMSO-d6) δ = 12.83 (s, 1H), 8.34 (br s, 3H), 8.13 (s, 1H), 8.07 (d, J = 8.4 Hz, 1H), 7.74 (d, J = 1.2 Hz, 1H), 7.52 (dd, J = 1.6, 8.4 Hz, 1H), 7.40- 7.34 (m, 2H), 7.33-7.28 (m, 2H), 4.27 (br s, 2H), 3.73 (s, 3H), 2.42 (s, 3H) |
| 4-98 | 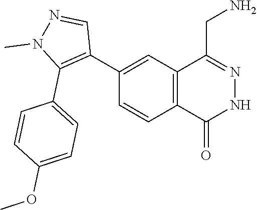 [SMILES: COc1ccc(-c2c(-c3ccc4c(=O)[nH]nc(CN)c4c3)cnn2C)cc1] | 4A | 4-5 | 4-(aminomethyl)-6-(5-(4-methoxyphenyl)-1- methyl-1H-pyrazol-4-yl)phthalazin-1(2H)-one LCMS [M + 1]⁺ = 362.1; ¹H NMR (400 MHz, DMSO-d6) δ = 12.83 (s, 1H), 8.36 (br s, 3H), 8.12 (s, 1H), 8.07 (d, J = 8.4 Hz, 1H), 7.77 (d, J = 1.2 Hz, 1H), 7.52 (dd, J = 1.6, 8.4 Hz, 1H), 7.38- 7.31 (m, 2H), 7.15-7.06 (m, 2H), 4.30 (br d, J = 4.0 Hz, 2H), 3.84 (s, 3H), 3.72 (s, 3H). |
| 4-99 | 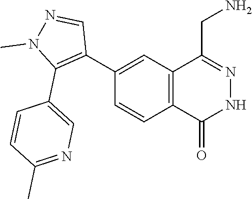 [SMILES: Cc1ccc(-c2c(-c3ccc4c(=O)[nH]nc(CN)c4c3)cnn2C)cn1] | 4A | 4-1 | 4-(aminomethyl)-6-(1-methyl-5-(6-methylpyridin- 3-yl)-1H-pyrazol-4-yl)phthalazin-1(2H)-one LCMS [M + 1]⁺ = 347.1; ¹H NMR (400 MHz, DMSO-d6) δ = 12.86 (s, 1H), 8.73 (s, 1H), 8.58 (br s, 3H), 8.22 (s, 1H), 8.17 (br d, J = 7.2 Hz, 1H), 8.10 (d, J = 8.4 Hz, 1H), 7.83 (d, J = 1.2 Hz, 1H), 7.77 (br d, J = 8.4 Hz, 1H), 7.52 (dd, J = 1.2, 8.4 Hz, 1H), 4.31 (br d, J = 5.6 Hz, 2H), 3.81 (s, 3H), 2.73 (s, 3H) |
| 4-100 | 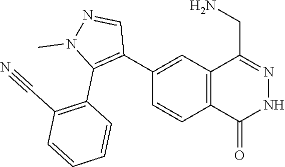 [SMILES: Cn1ncc(-c2ccc3c(=O)[nH]nc(CN)c3c2)c1-c1ccccc1C#N] | 4A | 4-4 | 2-(4-(4-(aminomethyl)-1-oxo-1,2- dihydrophthalazin-6-yl)-1-methyl-1H-pyrazol-5- yl)benzonitrile LCMS [M + 1]⁺ = 357.1; ¹H NMR (400 MHz, DMSO-d6) δ = 12.86 (s, 1H), 8.36 (br s, 3H), 8.28 (s, 1H), 8.09(m, 2H), 7.99-7.89 (td, J = 1.2 Hz, 7.6 Hz, 1H), 7.80 (t, J = 7.6 Hz, 2H), 7.68 (d, J = 1.2 Hz, 1H), 7.45 (dd, J = 1.6, 8.4 Hz, 1H), 4.24 (br d, J = 12.4 Hz, 2H), 3.74 (s, 3H). |
| 4-101 | 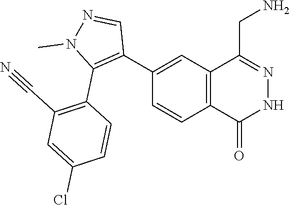 [SMILES: Cn1ncc(-c2ccc3c(=O)[nH]nc(CN)c3c2)c1-c1ccc(Cl)cc1C#N] | 4A | 4-6 | 2-(4-(4-(aminomethyl)-1-oxo-1,2- dihydrophthalazin-6-yl)-1-methyl-1H-pyrazol-5- yl)-5-chlorobenzonitrile LCMS [M + 1]⁺ = 391.0; ¹H NMR (400 MHz, DMSO-d6) δ = 12.86 (s, 1H), 8.59-8.44 (br s, 3H), 8.31 (s, 1H), 8.29 (d, J = 2.4 Hz, 1H), 8.11 (d, J = 8.4 Hz, 1H), 8.03 (dd, J = 2.4, 8.4 Hz, 1H), 7.84 (d, J = 1.2 Hz, 1H), 7.82 (d, J = 8.4 Hz, 1H), 7.38 (dd, J = 1.2, 8.4 Hz, 1H), 4.34 (d, J = 5.6 Hz, 2H), 3.74 (s, 3H) |
| 4-102 | 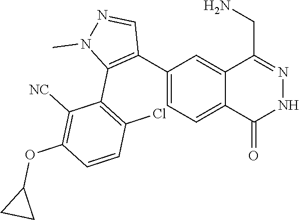 [SMILES: Cn1ncc(-c2ccc3c(=O)[nH]nc(CN)c3c2)c1-c1c(Cl)ccc(OC2CC2)c1C#N] | 4G | 4-6 | 2-(4-(4-(aminomethyl)-1-oxo-1,2- dihydrophthalazin-6-yl)-1-methyl-1H-pyrazol-5- yl)-3-chloro-6-cyclopropoxybenzonitrile LCMS [M + 1]⁺ = 447.1; ¹H NMR (400 MHz, DMSO-d₆) δ = 12.86 (s, 1H), 8.52 (br s, 3H), 8.39 (s, 1H), 8.13 (d, J = 8.4 Hz, 1H), 8.10 (d, J = 9.2 Hz, 1H), 7.81 (d, J = 9.2 Hz, 1H), 7.75 (d, J = 1.2 Hz, 1H), 7.42 (dd, J = 1.6, 8.4 Hz, 1H), 4.29 (br s, 2H), 4.24-4.17 (m, 1H), 3.69 (s, 3H), 0.90 (br dd, J = 3.4, 5.9 Hz, 2H), 0.87-0.74 (m, 2H) |
| 4-103 | 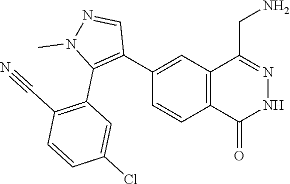 [SMILES: Cn1ncc(-c2ccc3c(=O)[nH]nc(CN)c3c2)c1-c1cc(Cl)ccc1C#N] | 4A | 4-6 | 2-(4-(4-(aminomethyl)-1-oxo-1,2- dihydrophthalazin-6-yl)-1-methyl-1H-pyrazol-5- yl)-4-chlorobenzonitrile LCMS [M + 1]⁺ = 391.2; ¹H NMR (400 MHz, DMSO-d6) δ = 12.86 (s, 1H), 8.50 (br s, 3H), 8.31 (s, 1H), 8.11 (m, 2H), 8.01 (d, J = 2.0 Hz, 1H), 7.91 (dd, J = 2.0, 8.6 Hz, 1H), 7.78 (d, J = 1.6 Hz, 1H), 7.42 (dd, J = 1.6, 8.4 Hz, 1H), 4.29 (d, J = 6.4 Hz, 2H), 3.77 (s, 3H) |
| 4-104 | 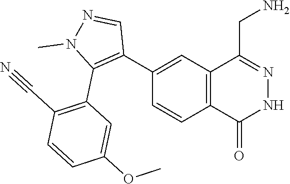 [SMILES: COc1ccc(C#N)c(-c2c(-c3ccc4c(=O)[nH]nc(CN)c4c3)cnn2C)c1] | 4A | 4-6 | 2-(4-(4-(aminomethyl)-1-oxo-1,2- dihydrophthalazin-6-yl)-1-methyl-1H-pyrazol-5- yl)-4-methoxybenzonitrile LCMS [M + 1]⁺ = 387.1; ¹H NMR (400 MHz, DMSO-d6) δ = 12.86 (s, 1H), 8.58-8.45 (s, 3H), 8.30 (s, 1H), 8.10 (d, J = 8.4 Hz, 1H), 7.99 (d, J = 8.8 Hz, 1H), 7.74 (d, J = 1.6 Hz, 1H), 7.47 (dd, J = 1.6, 8.4 Hz, 1H), 7.37-7.36 (m, 1H), 7.35-7.32 (m, 1H), 4.34-4.19 (m, 2H), 3.92 (s, 3H), 3.74 (s, 3H) |
| 4-105 | 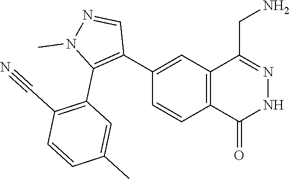 [SMILES: Cc1ccc(C#N)c(-c2c(-c3ccc4c(=O)[nH]nc(CN)c4c3)cnn2C)c1] | 4A | 4-4 | 2-(4-(4-(aminomethyl)-1-oxo-1,2- dihydrophthalazin-6-yl)-1-methyl-1H-pyrazol-5- yl)-4-methylbenzonitrile LCMS [M + 1]⁺ = 371.1; ¹H NMR (400 MHz, MeOD) δ = 8.20 (d, J = 8.4 Hz, 1H), 8.14 (s, 1H), 7.83 (d, J = 8.0 Hz, 1H), 7.70 (d, J = 1.6 Hz, 1H), 7.63-7.59 (m, 1H), 7.59 (s, 1H), 7.54 (dd, J = 1.6, 8.4 Hz, 1H), 4.45-4.29 (m, 2H), 3.81 (s, 3H), 2.54 (s, 3H) |
| 4-106 | 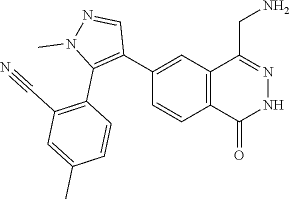 [SMILES: Cc1ccc(-c2c(-c3ccc4c(=O)[nH]nc(CN)c4c3)cnn2C)c(C#N)c1] | 4A | 4-4 | 2-(4-(4-(aminomethyl)-1-oxo-1,2- dihydrophthalazin-6-yl)-1-methyl-1H-pyrazol-5- yl)-5-methylbenzonitrile LCMS [M + 1]⁺ = 371.2; ¹H NMR (400 MHz, DMSO-d6) δ = 12.85 (s, 1H), 8.44 (br s, 3H), 8.26 (s, 1H), 8.09 (d, J = 8.4 Hz, 1H), 7.90 (s, 1H), 7.79-7.72 (m, 1H), 7.71-7.59 (m, 2H), 7.45 (br d, J = 8.4 Hz, 1H), 4.31 (d, J = 11.2 Hz, 2H), 3.72 (s, 3H), 2.47 (s, 3H) |
| 4-107 | 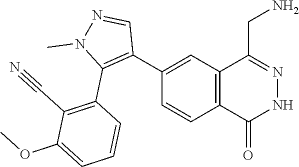 [SMILES: COc1cccc(-c2c(-c3ccc4c(=O)[nH]nc(CN)c4c3)cnn2C)c1C#N] | 4A | 4-4 | 2-(4-(4-(aminomethyl)-1-oxo-1,2- dihydrophthalazin-6-yl)-1-methyl-1H-pyrazol-5- yl)-6-methoxybenzonitrile LCMS [M + 1]⁺ = 387.2; ¹H NMR (400 MHz, DMSO-d6) δ = 12.86 (s, 1H), 8.59 (br s, 3H), 8.31 (s, 1H), 8.09 (d, J = 8.4 Hz, 1H), 7.89 (d, 8.4 Hz, 1H), 7.71 (d, J = 1.2 Hz, 1H), 7.52-7.46 (m, 2H), 7.28 (d, J = 7.6 Hz, 1H), 4.24 (br d, J = 5.6 Hz, 2H), 4.00 (s, 3H), 3.72 (s, 3H) |
| 4-108 | 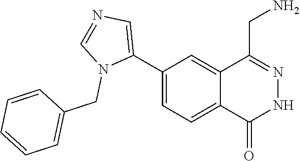 [SMILES: NCc1n[nH]c(=O)c2ccc(-c3cncn3Cc3ccccc3)cc12] | 4B | 4-7 | 4-(aminomethyl)-6-(1-benzyl-1H-imidazol-5- yl)phthalazin-1(2H)-one LCMS [M + 1]⁺ = 332. 1; ¹H NMR (400 MHz, MeOD) δ = 8.35 (d, J = 8.4 Hz, 1H), 7.99 (s, 1H), 7.88 (dd, J = 1.6, 8.4Hz, 1H), 7.76 (d, J = 1.6 Hz, 1H), 7.37 (d, J = 0.8 Hz, 1H), 7.34-7.24 (m, 3H), 7.02 (d, J = 6.8 Hz, 2H), 5.45 (s, 2H), 3.92 (s, 2H) |
| 4-109 | 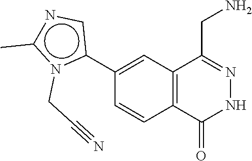 | 4A | 4-3 | 2-(5-(4-(aminomethyl)-1-oxo-1,2- dihydrophthalazin-6-yl)-2-methyl-1H-imidazol-1- yl)acetonitrile ¹H NMR (400 MHz, MeOD) δ = 8.59 (d, J = 8.4 Hz, 1H), 8.13 (d, J = 1.2 Hz, 1H), 8.07 (dd, J = 1.6, 8.4 Hz, 1H), 7.75 (s, 1H), 5.45 (s, 2H), 4.65 (s, 2H), 2.83 (s, 3H) |
| 4-110 | 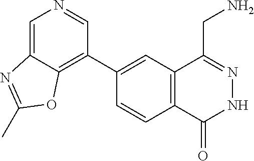 [SMILES: Cc1nc2cncc(-c3ccc4c(=O)[nH]nc(CN)c4c3)c2o1] | 4B | 4-2 | 4-(aminomethyl)-6-(2-methyloxazolo[4,5- c]pyridin-7-yl)phthalazin-1(2H)-one LCMS [M + 1]⁺ = 308.3; ¹H NMR (400 MHz, DMSO-d6) δ = 12.59 (br s, 1H), 9.05 (br s, 1H), 9.01 (br s, 1H), 8.58 (br s, 1H), 8.41 (br s, 1H), 8.38 (br s, 1H), 4.10 (d, J = 5.6Hz, 2H), 2.73 (s, 3H) |
| 4-111 | 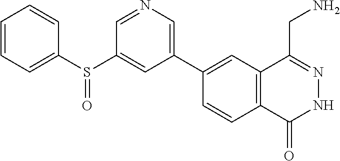 [SMILES: NCc1n[nH]c(=O)c2ccc(-c3cncc(S(=O)c4ccccc4)c3)cc12] | 4A | 4-6 | 4-(aminomethyl)-6-(5-(phenylsulfinyl)pyridin-3- yl)phthalazin-1(2H)-one LCMS [M + 1]⁺ = 377.0; ¹H NMR (400 MHz, DMSO-d6) δ = 13.0 (s, 1H), 9.27 (d, J = 2.0 Hz, 1H), 8.97 (d, J = 2.0 Hz, 1H), 8.72-8.66 (m, 1H), 8.65-8.45 (br s, 3H), 8.44-8.37 (m, 1H), 8.36- 8.32 (m, 1H), 8.30 (dd, J = 1.6, 8.4 Hz, 1H), 7.90 (d, J = 1.6 Hz, 1H), 7.89-7.84 (m, 1H), 7.62- 7.52(m, 3H), 4.59 (br d, J = 5.6 Hz, 2H) |
| 4-112 | 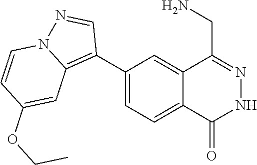 [SMILES: CCOc1ccn2ncc(-c3ccc4c(=O)[nH]nc(CN)c4c3)c2c1] | 4B | 4-4 | 4-(aminomethyl)-6-(5-ethoxypyrazolo[1,5- a]pyridin-3-yl)phthalazin-1(2H)-one LCMS [M + 1]⁺ = 336.2; ¹H NMR (400 MHz, DMSO-d6) δ = 12.86 (s, 1H), 8.68 (d, J = 7.6 Hz, 1H), 8.62 (m, 4H), 8.32-8.29 (m, 1H), 8.25- 8.21 (m, 1H), 8.09 (s, 1H), 7.41 (d, J = 2.4 Hz, 1H), 6.73 (dd, J = 2.4, 7.6 Hz, 1H), 4.58 (br d, J = 5.6 Hz, 2H), 4.24 (q, J = 6.8 Hz, 2H), 1.41 (t, J = 6.8 Hz, 3H) |
| 4-113 | 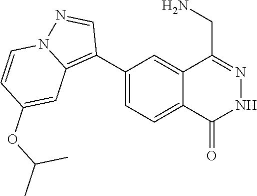 [SMILES: CC(C)Oc1ccn2ncc(-c3ccc4c(=O)[nH]nc(CN)c4c3)c2c1] | 4B | 4-4 | 4-(aminomethyl)-6-(5-isopropoxypyrazolo[1,5- a]pyridin-3-yl)phthalazin-1(2H)-one LCMS [M + 1]⁺ = 350.2; ¹H NMR (400 MHz, DMSO-d6) δ = 12.84 (s, 1H), 8.68 (d, J = 7.6 Hz, 1H), 8.60 (s, 4H), 8.33-8.28 (m, 1H), 8.21 (dd, J = 1.2, 8.4 Hz, 1H), 8.08 (s, 1H), 7.42 (d, J = 2.4 Hz, 1H), 6.71 (dd, J = 2.4, 7.6 Hz, 1H), 4.94 (td, J = 6.0, 12.0 Hz, 1H), 4.58 (br d, J = 5.2 Hz, 2H), 1.37 (s, 3H), 1.36 (s, 3H) |
| 4-114 | 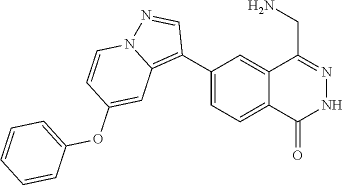 [SMILES: NCc1n[nH]c(=O)c2ccc(-c3cnn4ccc(Oc5ccccc5)cc34)cc12] | 4B | 4-4 | 4-(aminomethyl)-6-(5-phenoxypyrazolo[1,5- a]pyridin-3-yl)phthalazin-1(2H)-one LCMS [M + 1]⁺ = 384.1; ¹H NMR (400 MHz, DMSO-d6) δ = 12.86 (s, 1H), 8.84 (d, J = 7.6 Hz, 1H), 8.66 (s, 1H), 8.43 (br s, 3H), 8.28 (d, J = 8.4 Hz, 1H), 8.16 (br d, J = 7.6 Hz, 1H), 8.01 (d, J = 1.2 Hz, 1H), 7.63 (br s, 1H), 7.53-7.46 (m, 2H), 7.29-7.25 (m, 1H), 7.24-7.21 (m, 2H), 6.81 (dd, J = 2.4, 7.6 Hz, 1H), 4.53 (br s, 2H). |
| 4-115 | 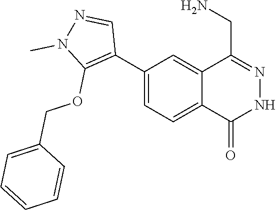 [SMILES: Cn1ncc(-c2ccc3c(=O)[nH]nc(CN)c3c2)c1OCc1ccccc1] | 4B | 4-4 | 4-(aminomethyl)-6-(5-(benzyloxy)-1-methyl-1H- pyrazol-4-yl)phthalazin-1(2H)-one LCMS [M + 1]⁺ = 362.1; ¹H NMR (400 MHz, DMSO-d6) δ = 12.87 (s, 1H), 8.41 (br s, 3H), 8.27 (d, J = 8.4 Hz, 1H), 8.12 (d, J = 8.4 Hz, 1H), 8.05 (s, 1H), 7.97 (d, J = 0.8 Hz, 1H), 7.43-7.36 (m, 5H), 5.07 (s, 2H), 4.45 (br s, 2H), 3.61 (s, 3H) |
| 4-116 | 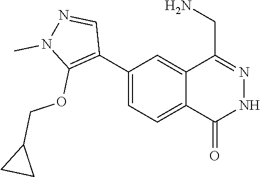 [SMILES: Cn1ncc(-c2ccc3c(=O)[nH]nc(CN)c3c2)c1OCC1CC1] | 4A | 4-2 | 4-(aminomethyl)-6-(5-(cyclopropylmethoxy)-1- methyl-1H-pyrazol-4-yl)phthalazin-1(2H)-one LCMS [M + 1]⁺ = 326.2; ¹H NMR (400 MHz, DMSO-d6) δ = 12.78 (br s, 1H), 8.28 (d, J = 8.4 Hz, 1H), 8.14 (dd, J = 1.6, 8.4 Hz, 1H), 8.06 (s, 1H), 8.03 (d, J = 1.2 Hz, 1H), 8.00-7.43 (br s, 2H), 4.43 (s, 2H), 3.90 (d, J = 7.6 Hz, 2H), 3.75 (s, 3H), 1.26-1.21 (m, 1H), 0.53 (m, 2H), 0.27- 0.20 (m, 2H) |
| 4-117 | 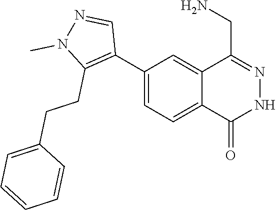 [SMILES: Cn1ncc(-c2ccc3c(=O)[nH]nc(CN)c3c2)c1CCc1ccccc1] | 4B | 4-4 | 4-(aminomethyl)-6-(1-methyl-5-phenethyl-1H- pyrazol-4-yl)phthalazin-1(2H)-one LCMS [M + 1]⁺ = 360.3; ¹H NMR (400 MHz, DMSO-d6) δ = 12.88 (s, 1H), 8.63 (br s, 3H), 8.26 (d, J = 8.4 Hz, 1H), 7.89 (s, 1H), 7.87 (dd, J = 1.2, 8.4 Hz, 1H), 7.82 (s, 1H), 7.23 (d, J = 7.6 Hz, 2H), 7.19-7.13 (m, 3H), 4.50 (br d, J = 5.6 Hz, 2H), 3.76 (s, 3H), 3.19 (brt, J = 7.6 Hz, 2H), 2.86 (br t, J = 7.6 Hz, 2H) |
| 4-118 | 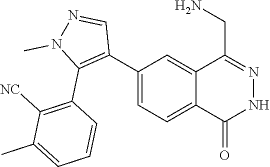 [SMILES: Cc1cccc(-c2c(-c3ccc4c(=O)[nH]nc(CN)c4c3)cnn2C)c1C#N] | 4A | 4-5 | 2-(4-(4-(aminomethyl)-1-oxo-1,2- dihydrophthalazin-6-yl)-1-methyl-1H-pyrazol-5- yl)-6-methylbenzonitrile LCMS [M + 1]⁺ = 371.1; ¹H NMR (400 MHz, DMSO-d6) δ = 12.85 (s, 1H), 8.43-8.29 (br s, 3H), 8.27 (s, 1H), 8.08 (d, J = 8.4 Hz, 1H), 7.83- 7.77 (m, 1H), 7.72-7.66 (m, 2H), 7.55 (d, J = 7.2 Hz, 1H), 7.46 (dd, J = 1.6, 8.4 Hz, 1H), 4.24 (br s, 2H), 3.70 (s, 3H), 2.55 (s, 3H) |
| 4-119 | 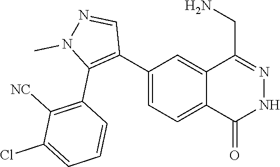 | 4A | 4-5 | 2-(4-(4-(aminomethyl)-1-oxo-1,2- dihydrophthalazin-6-yl)-1-methyl-1H-pyrazol-5- yl)-6-chlorobenzonitrile LCMS [M + 1]⁺ = 298.0; ¹H NMR (400 MHz, DMSO-d6) δ = 7.98 (dd, J = 1.2, 6.8 Hz, 1H), 7.94 (d, J = 7.6 Hz, 1H), 7.77 (s, 1H), 7.69 (dd, J = 1.2 Hz, 1H), 3.75 (s, 3H) |
| 4-120 | 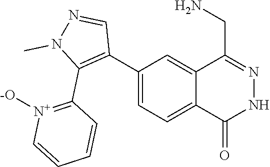 | 4B | 4-2 | 2-(4-(4-(aminomethyl)-1-oxo-1,2- dihydrophthalazin-6-yl)-1-methyl-1H-pyrazol-5- yl)pyridine 1-oxide LCMS [M + 1]⁺ = 349.3; ¹H NMR (400 MHz, DMSO-d6) δ = 12.72 (br s, 1H), 8.54 (d, J = 6.4 Hz, 1H), 8.19 (s, 1H), 8.13 (d. J = 8.4 Hz, 1H), 7.78 (d, J = 1.2 Hz, 1H), 7.69-7.61 (m, 1H), 7.59- 7.52 (m, 2H), 7.48-7.41 (td, J = 0.8, 7.6 Hz, 1H), 6.97 (br s, 2H), 4.14 (br d, J = 4.8 Hz, 2H), 3.74 (s, 3H) |
| 4-121 | 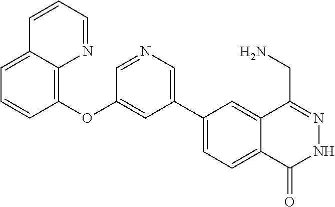 [SMILES: NCc1n[nH]c(=O)c2ccc(-c3cncc(Oc4cccc5cccnc45)c3)cc12] | 4B | 4-4 | 4-(aminomethyl)-6-(5-(quinolin-8-yloxy)pyridin- 3-yl)phthalazin-1(2H)-one LCMS [M + 1]⁺ = 396.1; ¹H NMR (400 MHz, DMSO-d6) δ = 12.98 (s, 1H), 8.88 (d, J = 2.0 Hz, 1H), 8.87 (dd, J = 1.6, 4.4 Hz, 1H), 8.49 (dd, J = 1.2, 8.4 Hz, 1H), 8.43-8.33 (m, 4H), 8.28 (d, J = 2.8 Hz, 2H), 8.24 (d, J = 8.4 Hz, 1H), 8.05-8.01 (t, J = 2.0 Hz, 1H), 7.94-7.90 (dd, J = 1.2, 8.0 Hz 1H), 7.67 (t, J = 8.0 Hz, 1H), 7.62 (dd, J = 4.0, 8.4 Hz, 1H), 7.55 (dd, J = 1.2, 7.6 Hz, 1H), 4.61- 4.57 (m, 2H) |
| 4-122 | 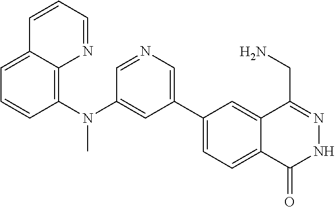 [SMILES: CN(c1cncc(-c2ccc3c(=O)[nH]nc(CN)c3c2)c1)c1cccc2cccnc12] | 4B | 4-4 | 4-(aminomethyl)-6-(5-(methyl(quinolin-8- yl)amino)pyridin-3-yl)phthalazin-1(2H)-one LCMS [M + 1]⁺ = 409.1; ¹H NMR (400 MHz, DMSO-d6) δ = 13.04 (s, 1H), 8.90 (dd, J = 1.6, 4.4 Hz, 1H), 8.76 (s, 1H), 8.73-8.60 (m, 4H), 8.41-8.37 (m, 2H), 8.29-8.25 (m, 1H), 8.17 (dd, J = 0.8. 8.4 Hz, 1H), 8.11 (s, 1H), 7.98 (dd, J = 1.2, 7.2 Hz, 1H), 7.84 (d, J = 8.0 Hz, 1H), 7.82 (d, J = 2.4 Hz, 1H), 7.72 (dd, J = 4.4, 8.4 Hz, 1H), 4.57 (br d, J = 5.6 Hz, 2H), 3.65 (s, 3H) |
| 4-123 | 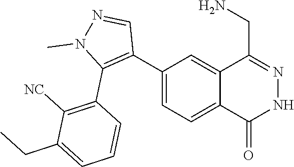 [SMILES: CCc1cccc(-c2c(-c3ccc4c(=O)[nH]nc(CN)c4c3)cnn2C)c1C#N] | 4B | 4-6 | 2-(4-(4-(aminomethyl)-1-oxo-1,2- dihydrophthalazin-6-yl)-1-methyl-1H-pyrazol-5- yl)-6-ethylbenzonitrile LCMS [M + 1]⁺ = 385.2; ¹H NMR (400 MHz, DMSO-d6) δ = 12.84 (s, 1H), 8.54 (br s, 3H), 8.32 (s, 1H), 8.07 (d, J = 8.4 Hz, 1H), 7.87 (t, J = 8.0 Hz, 1H), 7.73 (d. J = 7.6 Hz, 1H), 7.68 (s, 1H), 7.60 (d, J = 7.2 Hz, 1H), 7.48-7.42 (m, 1H), 4.20 (brd, J = 11.6 Hz, 2H), 3.72 (s, 3H), 2.86 (q, J = 7.6 Hz, 2H), 1.23 (t, J = 7.6 Hz, 3H) |
| 4-124 | 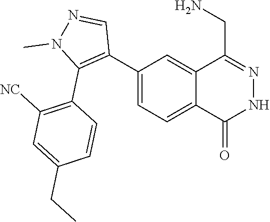 [SMILES: CCc1ccc(-c2c(-c3ccc4c(=O)[nH]nc(CN)c4c3)cnn2C)c(C#N)c1] | 4B | 4-5 | 2-(4-(4-(aminomethyl)-1-oxo-1,2- dihydrophthalazin-6-yl)-1-methyl-1H-pyrazol-5- yl)-5-ethylbenzonitrile LCMS [M + 1]⁺ = 385.2; ¹H NMR (400 MHz, DMSO-d6) δ = 12.86 (s, 1H), 8.36 (m, 3H), 8.25 (s, 1H), 8.09 (d, J = 8.4 Hz, 1H), 7.93 (s, 1H), 7.78 (dd, J = 1.6, 8.0 Hz, 1H), 7.70 (br s, 1H), 7.69- 7.66 (m, 1H), 7.43 (br d, J = 8.4 Hz, 1H), 4.36 (d, J = 14.4 Hz, 2H), 3,73 (s, 3H), 2.78 (q, J = 7.6 Hz, 2H), 1.27 (t, J = 7.6 Hz, 3H) |
| 4-125 | 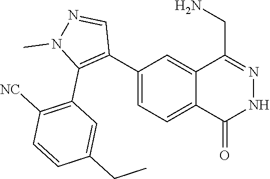 [SMILES: CCc1ccc(C#N)c(-c2c(-c3ccc4c(=O)[nH]nc(CN)c4c3)cnn2C)c1] | 4B | 4-5 | 2-(4-(4-(aminomethyl)-1-oxo-1,2- dihydrophthalazin-6-yl)-1-methyl-1H-pyrazol-5- yl)-4-ethylbenzonitrile LCMS [M + 1]⁺ = 385.2; ¹H NMR (400 MHz, DMSO-d6) δ = 12.85 (s, 1H), 8.50-8.29 (m, 3H), 8.27 (s, 1H), 8.10 (d, J = 8.4 Hz, 1H), 7.97 (d, J = 8.4 Hz, 1H), 7.71-7.63 (m, 2H), 7.63 (s, 1H), 7.47 (br d, J = 8.4 Hz, 1H), 4.35-4.10 (d, J = 9.6 Hz, 2H), 3.73 (s, 3H), 2.78 (q, J = 7.6Hz, 2H), 1.22 (t, J = 7.6 Hz, 3H) |
| 4-126 | 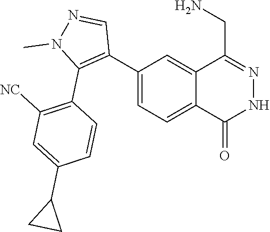 [SMILES: Cn1ncc(-c2ccc3c(=O)[nH]nc(CN)c3c2)c1-c1ccc(C2CC2)cc1C#N] | 4B | 4-5 | 2-(4-(4-(aminomethyl)-1-oxo-1,2- dihydrophthalazin-6-yl)-1-methyl-1H-pyrazol-5- yl)-5-cyclopropylbenzonitrile LCMS [M + 1]⁺ = 397.2; ¹H NMR (400 MHz, MeOD) δ = 8.20 (d, J = 8.4 Hz, 1H), 8.11 (s, 1H), 7.74 ( s, 1H), 7.64 (d, J = 1.2 Hz, 1H), 7.62-7.56 (m, 2H), 7.50 (br d, J = 8.4 Hz, 1H), 4.49-4.29 (m, 2H), 3.79 (s, 3H), 2.10 (s, 1H), 1.23-1.09 (m, 2H), 0.95-0.79 (m, 2H) |
| 4-127 | 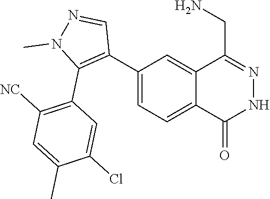 [SMILES: Cc1cc(C#N)c(-c2c(-c3ccc4c(=O)[nH]nc(CN)c4c3)cnn2C)cc1Cl] | 4B | 4-4 | 2-(4-(4-(aminomethyl)-1-oxo-1,2- dihydrophthalazin-6-yl)-1-methyl-1H-pyrazol-5- yl)-4-chloro-5-methylbenzonitrile LCMS [M + 1]⁺ = 405.2; ¹H NMR (400 MHz, DMSO-d6) δ = 12.87 (s, 1H), 8.36 (br s, 3H), 8.26 (s, 1H), 8.15-8.07 (m, 2H), 7.97 (s, 1H), 7.78 (s, 1H), 7.42 (dd, J = 1.6, 8.4 Hz, 1H), 4.34 (d, J = 10.8 Hz, 1H), 3.75 (s, 3H), 2.48 (s, 3H) |
| 4-128 | 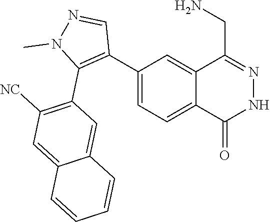 [SMILES: Cn1ncc(-c2ccc3c(=O)[nH]nc(CN)c3c2)c1-c1cc2ccccc2cc1C#N] | 4B | 4-4 | 3-(4-(4-(aminomethyl)-1-oxo-1,2- dihydrophthalazin-6-yl)-1-methyl-1H-pyrazol-5- yl)-2-naphthonitrile LCMS [M + 1]⁺ = 407.1; ¹H NMR (400 MHz, DMSO-d6) δ = 12.83 (s, 1H), 8.82 (s, 1H), 8.37 (s, 1H), 8.32 (s, 4H), 8.22-8.18 (m, 1H), 8.15- 8.11 (m, 1H), 8.02 (d, J = 8.4 Hz, 1H), 7.90 (d, J = 1.6 Hz, 1H), 7.87-7.79 (m, 2H), 7.36 (dd, J = 1.2, 8.0 Hz, 1H), 4.31 (br d, J = 0.8 Hz, 2H), 3.77 (s, 3H) |
| 4-129 | 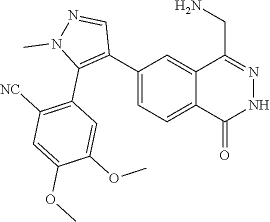 [SMILES: COc1cc(C#N)c(-c2c(-c3ccc4c(=O)[nH]nc(CN)c4c3)cnn2C)cc1OC] | 4B | 4-4 | 2-(4-(4-(aminomethyl)-1-oxo-1,2- dihydrophthalazin-6-yl)-1-methyl-1H-pyrazol-5- yl)-4,5-dimethoxybenzonitrile LCMS [M + 1]⁺ = 417.1; ¹H NMR (400 MHz, DMSO-d6) δ = 12.85 (s, 1H), 8.37 (br s, 3H), 8.26 (s, 1H), 8.11 (d, J = 8.4 Hz, 1H), 7.80 (d, J = 1.2 Hz, 1H), 7.57 (s, 1H), 7.47 (dd, J = 1.6, 8.4 Hz, 1H), 7.35 (s, 1H), 4.40-4.26 (br d, J = 5.6 Hz, 2H), 3.90 (s, 3H), 3.88 (s, 3H), 3.74 (s, 3H) |
| 4-130 | 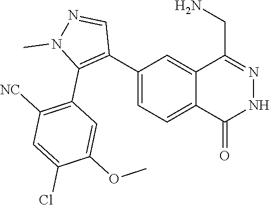 [SMILES: COc1cc(-c2c(-c3ccc4c(=O)[nH]nc(CN)c4c3)cnn2C)c(C#N)cc1Cl] | 4B | 4-4 | 2-(4-(4-(aminomethyl)-1-oxo-1,2- dihydrophthalazin-6-yl)-1-methyl-1H-pyrazol-5- yl)-5-chloro-4-methoxybenzonitrile LCMS [M + 1]⁺ = 421.1; ¹H NMR (400 MHz, DMSO-d6) δ = 12.87 (s, 1H), 8.37 (br s, 3H), 8.28 (s, 1H), 8.22 (s, 1H), 8.13 (d, J = 8.4 Hz, 1H), 7.85 (d, J = 1.2 Hz, 1H), 7.60 (s, 1H), 7.45 (dd, J = 1.6, 8.4 Hz, 1H), 4.45-4.29 (m, 2H), 4.01 (s, 3H), 3.77 (s, 3H) |
| 4-131 | 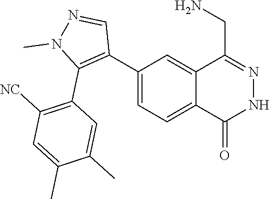 [SMILES: Cc1cc(C#N)c(-c2c(-c3ccc4c(=O)[nH]nc(CN)c4c3)cnn2C)cc1C] | 4B | 4-4 | 2-(4-(4-(aminomethyl)-1-oxo-1,2- dihydrophthalazin-6-yl)-1-methyl-1H-pyrazol-5~ yl)-4-5-dimethylbenzonitrile LCMS [M + 1]⁺ = 385.1; ¹H NMR (400 MHz, DMSO-d6) δ = 12.87 (s, 1H), 8.34 (br s, 3H), 8.27 (s, 1H), 8.09 (d, J = 8.4 Hz, 1H), 7.85 (s, 1H), 7.72 (d, J = 1.2 Hz, 1H), 7.58 (s, 1H), 7.44 (dd, J = 1.6, 8.4 Hz, 1H), 4.32 (d, J = 22.0 Hz, 2H), 3.71 (s, 3H), 2.39 (s, 3H), 2.38 (s, 3H) |
| 4-132 | 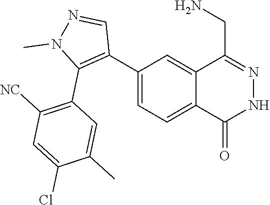 [SMILES: Cc1cc(-c2c(-c3ccc4c(=O)[nH]nc(CN)c4c3)cnn2C)c(C#N)cc1Cl] | 4B | 4-5 | 2-(4-(4-(aminomethyl)-1-oxo-1,2- dihydrophthalazin-6-yl)-1-methyl-1H-pyrazol-5- yl)-5-chloro-4-methylbenzonitrile LCMS [M + 1]⁺ = 405.2; ¹H NMR (400 MHz, MeOD) δ = 8.22 (d, J = 8.4 Hz, 1H), 8.12 (s, 1H), 7.99 (s, 1H), 7.77 (s, 1H), 7.71 (s, 1H), 7.51 (d, J = 8.4 Hz, 1H), 4.45 (d, J = 9.2 Hz, 2H), 3.81 (s, 3H), 2.55 (s, 3H) |
| 4-133 | 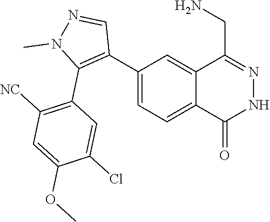 [SMILES: COc1cc(C#N)c(-c2c(-c3ccc4c(=O)[nH]nc(CN)c4c3)cnn2C)cc1Cl] | 4B | 4-6 | 2-(4-(4-(aminomethyl)-1-oxo-1,2- dihydrophthalazin-6-yl)-1-methyl-1H-pyrazol-5- yl)-4-chloro-5-methoxybenzonitrile LCMS [M + 1]⁺ = 421.1; ¹H NMR (400 MHz, DMSO-d6) δ = 12.86 (s, 1H), 8.43 (br s, 3H), 8.27 (s, 1H), 8.12 (d, J = 8.4 Hz, 1H), 7.97 (s, 1H), 7.86 (s, 1H), 7.84-7.82 (d, J = 1.6 Hz,, 1H), 7.41 (dd, J = 1.6, 8.4 Hz, 1H), 4.40-4.30 (m, 2H), 4.01 (s, 3H), 3.74 (s, 3H) |
| 4-134 | 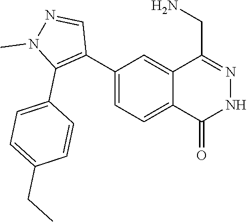 [SMILES: CCc1ccc(-c2c(-c3ccc4c(=O)[nH]nc(CN)c4c3)cnn2C)cc1] | 4B | 4-4 | 4-(aminomethyl)-6-(5-(4-ethylphenyl)-1-methyl- 1H-pyrazol-4-yl)phthalazin-1(2H)-one LCMS [M + 1]⁺ = 360.2; ¹H NMR (400 MHz, DMSO-d6) δ = 12.83 (s, 1H), 8.51-8.28 (br s, 3H), 8.12 (s, 1H), 8.07 (d, J = 8.4 Hz, 1H), 7.73 (d, J = 1.2 Hz, 1H), 7.52 (dd, J = 1.2, 8.4 Hz, 1H), 7.42-7.37 (m, 2H), 7.36-7.30 (m, 2H), 4.26 (d, J = 5.6 Hz, 2H), 3.74 (s, 3H), 2.72 (q, J = 7.6 Hz, 2H), 1.25 (t, J = 7.6 Hz, 3H) |
| 4-135 | 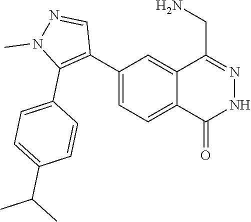 [SMILES: CC(C)c1ccc(-c2c(-c3ccc4c(=O)[nH]nc(CN)c4c3)cnn2C)cc1] | 4B | 4-5 | 4-(aminomethyl)-6-(5-(4-isopropylphenyl)-1- methyl-1H-pyrazol-4-yl)phthalazin-1(2H)-one LCMS [M + 1]⁺ = 374.2; ¹H NMR (400 MHz, MeOD) δ = 8.13 (d, J = 8.4 Hz, 1H), 8.02 (s, 1H), 7.66-7.60 (m, 2H), 7.44 (d, J = 8.4 Hz, 2H), 7.31 (d, J = 8.4 Hz, 2H), 4.27 (s, 2H), 3.77 (s, 3H), 3.07- 2.98 (m, 1H), 1.33 (s, 3H), 1.31 (s, 3H) |
| 4-136 | 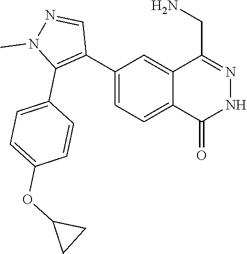 [SMILES: Cn1ncc(-c2ccc3c(=O)[nH]nc(CN)c3c2)c1-c1ccc(OC2CC2)cc1] | 4B | 4-4 | 4-(aminomethyl)-6-(5-(4-cyclopropoxyphenyl)-1- methyl-1H-pyrazol-4-yl)phthalazin-1(2H)-one LCMS [M + 1]⁺ = 388.2; ¹H NMR (400 MHz, DMSO-d6) δ = 12.83 (s, 1H), 8.40 (br s, 3H), 8.11 (s, 1H), 8.08 (d, J = 8.4 Hz, 1H), 7.76 (d, J = 1.2 Hz, 1H), 7.53 (dd, J = 1.6, 8.4 Hz, 1H), 7.35 (d, J = 8.8 Hz, 2H), 7.21 (d, J = 8.8 Hz, 2H), 4.29 (br s, 2H), 3.93 (td, J = 3.2, 6.0 Hz, 1H), 3.73 (s, 3H), 0.88-0.79 (m, 2H), 0.75-0.67 (m, 2H) |
| 4-137 | 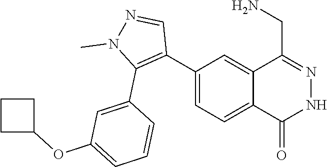 [SMILES: Cn1ncc(-c2ccc3c(=O)[nH]nc(CN)c3c2)c1-c1cccc(OC2CCC2)c1] | 4B | 4-4 | 4-(aminomethyl)-6-(5-(3-cyclobutoxyphenyl)-1- methyl-1H-pyrazol-4-yl)phthalazin-1(2H)-one LCMS [M + 1]⁺ = 402.2; ¹H NMR (400 MHz, DMSO-d6) δ = 12.84 (s, 1H), 8.36 (br s, 3H), 8.13 (s, 1H), 8.07 (d, J = 8.4 Hz, 1H), 7.73 (d, J = 1.2 Hz, 1H), 7.52 (dd, J = 1.6, 8.4 Hz, 1H), 7.44 (t, J = 8.0 Hz, 1H), 7.02 (dd, J = 2.0, 8.4 Hz, 1H), 6.96 (d, J = 7.6 Hz, 1H), 6.85-6.82 (m, 1H), 4.69 (t, J = 7.2 Hz, 1H), 4.27 (s, 2H), 3.75 (s, 3H), 2.37- 2.25 (m, 2H), 2.03-1.87 (m, 2H), 1.73 (q, J = 10.4 Hz, 1H), 1.64-1.50 (m, 1H) |
| 4-138 | 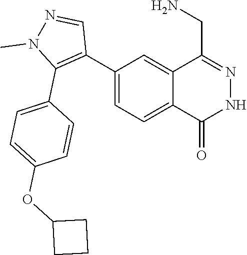 [SMILES: Cn1ncc(-c2ccc3c(=O)[nH]nc(CN)c3c2)c1-c1ccc(OC2CCC2)cc1] | 4B | 4-6 | 4-(aminomethyl)-6-(5-(4-cyclobutoxyphenyl)-1- methyl-1H-pyrazol-4-yl)phthalazin-1(2H)-one LCMS [M + 1]⁺ = 402.2; ¹H NMR (400 MHz, DMSO-d6) δ = 12.82 (s, 1H), 8.60-8.36 (br s, 3H), 8.12 (s, 1H), 8.06 (dd, J = 1.2, 8.4 Hz, 1H), 7.79 (s, 1H), 7.52-7.46 (m, 1H), 7.31 (d, J = 8.8 Hz, 2H), 7.04-6.94 (m, 2H), 4.77 (quin, J = 7.2 Hz, 1H), 4.29 (br d, J = 5.6 Hz, 2H), 3.72 (s, 3H), 2.47-2.41 (m, 2H), 2.17-2.03 (m, 2H), 1.81 (q, J = 10.0 Hz, 1H), 1.72-1.60 (m, 1H) |
| 4-139 | 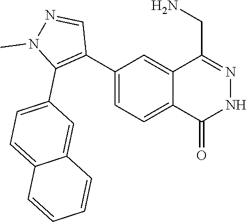 [SMILES: Cn1ncc(-c2ccc3c(=O)[nH]nc(CN)c3c2)c1-c1ccc2ccccc2c1] | 4B | 4-4 | 4-(aminomethyl)-6-(1-methyl-5-(naphthalen-2- yl)-1H-pyrazol-4-yl)phthalazin-1(2H)-one LCMS [M + 1]⁺ = 382.1; ¹H NMR (400 MHz, DMSO-d6) δ = 12.82 (s, 1H), 8.35 (s, 3H), 8.19 (s, 1H), 8.10-7.95 (m, 5H), 7.89 (s, 1H), 7.68- 7.57 (m, 2H), 7.48 (dd, J = 1.6, 8.8 Hz, 1H), 7.43 (d, J = 8.4 Hz, 1H), 4.31 (br s, 2H), 3.80 (s, 3H) |
| 4-140 | 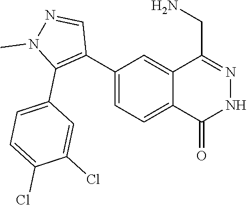 [SMILES: Cn1ncc(-c2ccc3c(=O)[nH]nc(CN)c3c2)c1-c1ccc(Cl)c(Cl)c1] | 4B | 4-4 | 4-(aminomethyl)-6-(5-(3,4-dichlorophenyl)-1- methyl-1H-pyrazol-4-yl)phthalazin-1(2H)-one LCMS [M + 1]⁺ = 400.1; ¹H NMR (400 MHz, DMSO-d6) δ = 12.86 (s, 1H), 8.36 (br s, 3H), 8.14- 8.10 (d, J = 8.4 Hz, 2H), 7.85 (d, J = 1.2 Hz, 1H), 7.79 (m, 2H), 7.47 (dd, J = 1.6, 8.4 Hz, 1H), 7.40 (dd, J = 2.0, 8.4 Hz, 1H), 4.37 (s, 2H), 3.78 (s, 3H) |
| 4-141 | 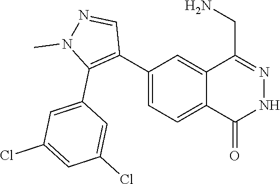 [SMILES: Cn1ncc(-c2ccc3c(=O)[nH]nc(CN)c3c2)c1-c1cc(Cl)cc(Cl)c1] | 4B | 4-4 | 4-(aminomethyl)-6-(5-(3,5-dichlorophenyl)-1- methyl-1H-pyrazol-4-yl)phthalazin-1(2H)-one LCMS [M + 1]⁺ = 400.0; ¹H NMR (400 MHz, MeOD) δ = 8.26 (br d, J = 8.4 Hz, 1H), 8.04 (s, 1H), 7.71 (d, J = 1.2 Hz, 1H), 7.70-7.62 (m, 2H), 7.46 (d, J = 1.2 Hz, 2H), 4.39 (s, 2H), 3.83 (s, 3H) |
| 4-142 | 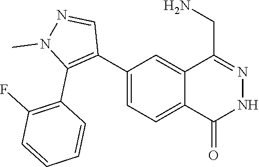 [SMILES: Cn1ncc(-c2ccc3c(=O)[nH]nc(CN)c3c2)c1-c1ccccc1F] | 4B | 4-4 | 4-(aminomethyl)-6-(5-(2-fluorophenyl)-1-methyl- 1H-pyrazol-4-yl)phthalazin-1(2H)-one LCMS [M + 1]⁺ = 350.2; ¹H NMR (400 MHz, DMSO-d6) δ = 12.85 (s, 1H), 8.40-8.28 (br s, 3H), 8.23 (s, 1H), 8.10 (d, J = 8.4 Hz, 1H), 7.75 (d, J = 1.2 Hz, 1H), 7.71-7.64 (m, 1H), 7.56- 7.50 (m, 2H), 7.49-7.40 (m, 2H), 4.28 (br d, J = 5.6 Hz, 2H), 3.73 (s, 3H) |
| 4-143 | 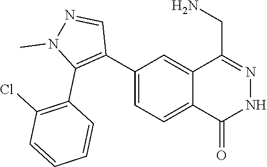 [SMILES: Cn1ncc(-c2ccc3c(=O)[nH]nc(CN)c3c2)c1-c1ccccc1Cl] | 4B | 4-4 | 4-(aminomethyl)-6-(5-(2-chlorophenyl)-1-methyl- 1H-pyrazol-4-yl)phthalazin-1(2H)-one LCMS [M + 1]⁺ = 366.1; ¹H NMR (400 MHz, DMSO-d6) δ = 12.83 (s, 1H), 8.42 (br s, 3H), 8.26 (s, 1H), 8.08 (d, J = 8.4 Hz, 1H), 7.76-7.72 (m, 1H), 7.68 (d, J = 1.6 Hz, 1H), 7.67-7.61 (m, 1H), 7.57-7.56 (m, 1H), 7.55 (d, J = 0.8 Hz, 1H), 7.49 (dd, J = 1.6, 8.4 Hz, 1H), 4.28-4.18 (m, 2H), 3.65 (s, 3H) |
| 4-144 | 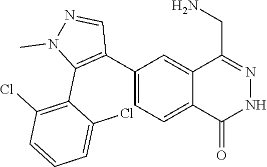 [SMILES: Cn1ncc(-c2ccc3c(=O)[nH]nc(CN)c3c2)c1-c1c(Cl)cccc1Cl] | 4B | 4-5 | 4-(aminomethyl)-6-(5-(2,6-dichlorophenyl)-1- methyl-1H-pyrazol-4-yl)phthalazin-1(2H)-one LCMS [M + 1]⁺ = 400.0; ¹H NMR (400 MHz, DMSO-d6) δ = 12.85 (s, 1H), 8.46-8.30 (m, 4H), 8.11 (d, J = 8.4 Hz, 1H), 7.79-7.75 (m, 2H), 7.74 (d, J = 1.2 Hz, IH), 7.72-7.65 (m, 1H), 7.43 (dd, J = 1.6, 8.4 Hz, 1H), 4.30 (br d, J = 5.2 Hz, 2H), 3.64 (s, 3H) |
| 4-145 | 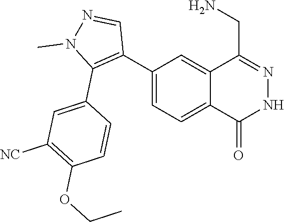 [SMILES: CCOc1ccc(-c2c(-c3ccc4c(=O)[nH]nc(CN)c4c3)cnn2C)cc1C#N] | 4B | 4-5 | 5-(4-(4-(aminomethyl)-1-oxo-1,2- dihydrophthalazin-6-yl)-1-methyl-1H-pyrazol-5- yl)-2-ethoxybenzonitrile LCMS [M + 1]⁺ = 401.2; ¹H NMR (400 MHz, DMSO-d6) δ = 12.85 (s, 1H), 8.40 (br s, 3H), 8.13 (s, 1H), 8.10 (d, J = 8.4 Hz, 1H), 7.88 (d, J = 2.0 Hz, 1H), 7.80 (s, 1H), 7.64 (dd, J = 2.0, 8.8 Hz, 1H), 7.48 (dd, J = 1.2, 8.4 Hz, 1H), 7.36 (d, J = 8.8 Hz, 1H), 4.34 (br s, 2H), 4.28 (q, J = 6.8 Hz, 2H), 3.75 (s, 3H), 1.41 (t, J = 6.8 Hz, 3H) |
| 4-146 | 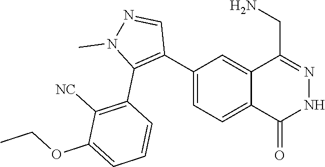 [SMILES: CCOc1cccc(-c2c(-c3ccc4c(=O)[nH]nc(CN)c4c3)cnn2C)c1C#N] | 4B | 4-4 | 2-(4-(4-(aminomethyl)-1-oxo-1,2- dihydrophthalazin-6-yl)-1-methyl-1H-pyrazol-5- yl)-6-ethoxybenzonitrile LCMS [M + 1]⁺ = 401.2; ¹H NMR (400 MHz, DMSO-d6) δ = 12.87 (s, 1H), 8.38 (br s, 3H), 8.27 (s, 1H), 8.09 (d, J = 8.4 Hz, 1H), 7.85 (dd, J = 7.6, 8.4 Hz, 1H), 7.76 (s, 1H), 7.46 (d, J = 8.8 Hz, 1H), 7.45-7.40 (m. 1H), 7.26 (d, J = 7.6 Hz, 1H), 4.38- 4.28 (m, 2H), 4.28-4.21 (m, 2H), 3.72 (s, 3H), 1.38 (t, J = 7.2 Hz, 3H) |
| 4-147 | 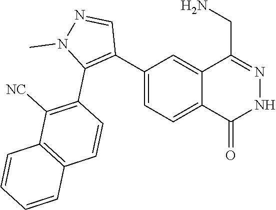 [SMILES: Cn1ncc(-c2ccc3c(=O)[nH]nc(CN)c3c2)c1-c1ccc2ccccc2c1C#N] | 4B | 4-4 | 2-(4-(4-(aminomethyl)-1-oxo-1,2- dibydrophthalazin-6-yl)-1-methyl-1H-pyrazol-5- yl)-1-naphthonitrile LCMS [M + 1]⁺ = 407.2; ¹H NMR (400 MHz, DMSO-d6) δ = 12.85 (s, 1H), 8.52 (d, J = 8.4 Hz, 1H), 8.41-8.30 (m, 4H), 8.28 (d, J = 7.6 Hz, 1H), 8.14 (d, J = 8.0 Hz, 1H), 8.01 (d, J = 8.0 Hz, 1H), 7.93-7.84 (m, 3H), 7.83 (d, J = 8.8 Hz, 1H), 7.33 (dd, J = 1.2, 8.4 Hz, 1H), 4.42-4.20 (m, 2H), 3.78 (s, 3H) |
| 4-148 | 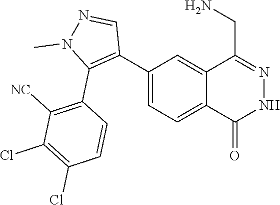 [SMILES: Cn1ncc(-c2ccc3c(=O)[nH]nc(CN)c3c2)c1-c1ccc(Cl)c(Cl)c1C#N] | 4B | 4-4 | 6-(4-(4-(aminomethyl)-1-oxo-1,2- dihydrophthalazin-6-yl)-1-methyl-1H-pyrazol-5- yl)-2,3-dichlorobenzonitrile LCMS [M + 1]⁺ = 425.0; ¹H NMR (400 MHz, DMSO-d6) δ = 12.89 (s, 1H), 8.36 (br s, 3H), 8.29 (s, 1H), 8.21 (d, J = 8.4 Hz, 1H), 8.14 (d, J = 8.4 Hz, 1H), 7.87 (d, J = 1.2 Hz, 1H), 7.79 (d, J = 8.4 Hz, 1H), 7.44 (dd, J = 1.6, 8.4 Hz, 1H), 4.40 (br s, 2H), 3.78 (s, 3H) |
| 4-149 | 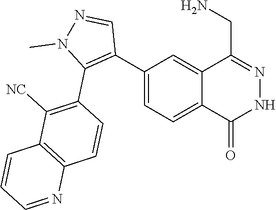 [SMILES: Cn1ncc(-c2ccc3c(=O)[nH]nc(CN)c3c2)c1-c1ccc2ncccc2c1C#N] | 4B | 4-4 | 6-(4-(4-(aminomethyl)-1-oxo-1,2- dihydrophthalazin-6-yl)-1-methyl-1H-pyrazol-5- yl)quinoline-5-carbonitrile LCMS [M + 1]⁺ = 408.1; ¹H NMR (400 MHz, DMSO-d6) δ = 12.84 (s, 1H), 9.20 (dd, J = 1.6, 4.0 Hz, 1H), 8.58-8.52 (m, 2H), 8.33 (s, 1H), 8.30 (br s, 3H), 8.06 (d, J = 8.8 Hz, 1H), 8.03 (d, J = 8.4 Hz, 1H), 7.88 (dd, J = 4.0, 8.4 Hz, 1H), 7.85 (d, J = 1.2 Hz, 1H), 7.39 (dd, J = 1.2, 8.4 Hz, 1H), 4.39-4.19 (m, 2H), 3.80 (s, 3H) |
| 4-150 | 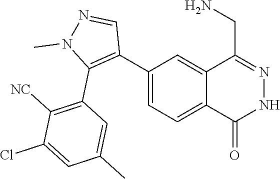 [SMILES: Cc1cc(Cl)c(C#N)c(-c2c(-c3ccc4c(=O)[nH]nc(CN)c4c3)cnn2C)c1] | 4B | 4-4 | 2-(4-(4-(aminomethyl)-1-oxo-1,2- dihydrophthalazin-6-yl)-1-methyl-1H-pyrazol-5- yl)-6-chloro-4-methylbenzonitrile LCMS [M + 1]⁺ = 405.1; ¹H NMR (400 MHz, DMSO-d6) δ = 8.23 (d, J = 8.0 Hz, 1H), 8.12 (s, 1H), 7.77 (d, J = 1.2 Hz, 1H), 7.73 (s, 1H), 7.55 (s, 1H), 7.52 (dd, J = 1.2, 8.0 Hz, 1H), 4.51-4.37 (m, 2H), 3.82 (s, 3H), 2.53 (s, 3H) |
| 4-151 | 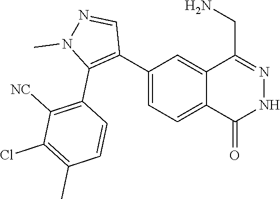 [SMILES: Cc1ccc(-c2c(-c3ccc4c(=O)[nH]nc(CN)c4c3)cnn2C)c(C#N)c1Cl] | 4B | 4-4 | 2-(4-(4-(aminomethyl)-1-oxo-1,2- dihydrophthalazin-6-yl)-1-methyl-1H-pyrazol-5- yl)-6-chloro-5-methylbenzonitrile LCMS [M + 1]⁺ = 405.1; ¹H NMR (400 MHz, DMSO-d6) δ = 12.87 (s, 1H), 8.53-8.33 (m, 3H), 8.28 (s, 1H), 8.12 (d, J = 8.4 Hz, 1H), 7.92 (d, J = 8.0Hz, 1H), 7,78 (s, 1H), 7.68 (d, J = 8.0 Hz, 1H), 7.45 (br d, J = 8.4 Hz, 1H), 4.23 (d, J = 12Hz, 2H), 3.75 (s, 3H), 3.33 (br s, 3H) |
| 4-152 | 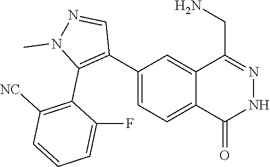 [SMILES: Cn1ncc(-c2ccc3c(=O)[nH]nc(CN)c3c2)c1-c1c(F)cccc1C#N] | 4B | 4-5 | 2-(4-(4-(aminomethyl)-1-oxo-1,2- dihydrophthalazin-6-yl)-1-methyl-1H-pyrazol-5- yl)-3-fluorobenzonitrile LCMS [M + 1]⁺ = 375.2; ¹H NMR (400 MHz, DMSO-d6) δ = 12.88 (s, 1H), 8.39 (br s, 3H), 8.35 (s, 1H), 8.13 (d, J = 8.4 Hz, 1H), 8.01-7.95 (m, 1H), 7.94-7.87 (m, 2H), 7.74 (s, 1H), 7.45 (br d, J = 8.4 Hz, 1H), 4.30 (br d, J = 7.2 Hz, 2H), 3.77 (s, 3H) |
| 4-153 | 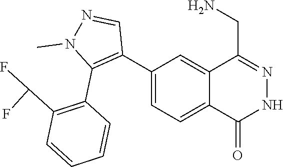 [SMILES: Cn1ncc(-c2ccc3c(=O)[nH]nc(CN)c3c2)c1-c1ccccc1C(F)F] | 4B | 4-4 | 4-(aminomethyl)-6-(5-(2- (difluoromethyl)phenyl)-1-methyl-1H-pyrazol-4- yl)phthalazin-1(2H)-one LCMS [M + 1]⁺ = 382.1; ¹H NMR (400 MHz, DMSO-d6) δ = 12.82 (s, 1H), 8.34 (br s, 3H), 8.28 (s, 1H), 8.04 (d, J = 8.4 Hz, 1H), 7.87-7.83 (m, 1H), 7.82-7.78 (m, 2H), 7.63-7.58 (m, 2H), 7.46 (d, J = 8.4 Hz, 1H), 6.77-6.46 (t, J = 54.4 Hz, 1H), 4.21-4.13 (m, 2H), 3.60 (s, 3H) |
| 4-154 | 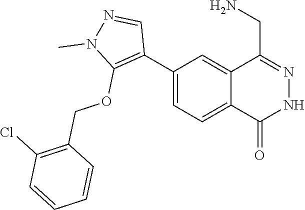 [SMILES: Cn1ncc(-c2ccc3c(=O)[nH]nc(CN)c3c2)c1OCc1ccccc1Cl] | 4B | 4-4 | 4-(aminomethyl)-6-(5-((2-chlorobenzyl)oxy)-1- methyl-1H-pyrazol-4-yl)phthalazin-1(2H)-one LCMS [M + 1]⁺ = 396.1; ¹H NMR (400 MHz, DMSO-d6) δ = 12.87 (s, 1H), 8.47-8.29 (br s, 3H), 8.24 (d, J = 8.4 Hz, 1H), 8.07 (dd, J = 1.6, 8.4 Hz, 1H), 8.03 (s, 1H), 7.93 (d, J = 1.6 Hz, 1H), 7.50-7.45 (m, 2H), 7.38 (dt, J = 2.0, 7.6 Hz, 1H), 7.36-7.30 (dt, J = 2.0, 7.6 Hz, 1H), 5.16 (s, 2H), 4.45 (s, 2H), 3.63 (s, 3H) |
| 4-155 | 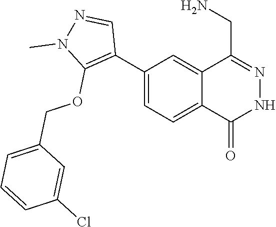 [SMILES: Cn1ncc(-c2ccc3c(=O)[nH]nc(CN)c3c2)c1OCc1cccc(Cl)c1] | 4B | 4-4 | 4-(aminomethyl)-6-(5-((3-chlorobenzyl)oxy)-1- methyl-1H-pyrazol-4-yl)phthalazin-1(2H)-one LCMS [M + 1]⁺ = 396.1; ¹H NMR (400 MHz, DMSO-d6) δ = 12.86 (s, 1H), 8.43-8.30 (br s, 3H), 8.25 (d, J = 8.4 Hz, 1H), 8.05 (dd, J = 1.6, 8.4 Hz, 1H), 8.02 (s, 1H), 7.92 (d, J = 1.6 Hz, 1H), 7.46 (d, J = 1.2 Hz, 1H), 7.40-7.35 (m, 3H), 5.08 (s, 2H), 4.45 (s, 2H), 3.67 (s, 3H) |
| 4-156 | 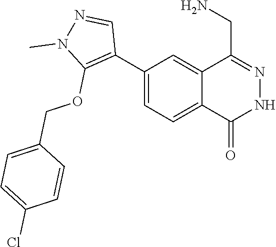 [SMILES: Cn1ncc(-c2ccc3c(=O)[nH]nc(CN)c3c2)c1OCc1ccc(Cl)cc1] | 4B | 4-4 | 4-(aminomethyl)-6-(5-((4-chlorobenzyl)oxy)-1- methyl-1H-pyrazol-4-yl)phthalazin-1(2H)-one LCMS [M + 1]⁺ = 396.1; ¹H NMR (400 MHz, DMSO-d6) δ = 12.86 (s, 1H), 8.53-8.32 (br s, 3H), 8.26 (d, J = 8.4 Hz, 1H), 8.06 (dd, J = 1.6, 8.4 Hz, 1H), 8.03 (s, 1H), 7.92 (d, J = 1.6 Hz, 1H), 7.44-7.39 (m, 4H), 5.07 (s, 2H), 4.47 (br s, 2H), 3.65 (s, 3H) |
| 4-157 | 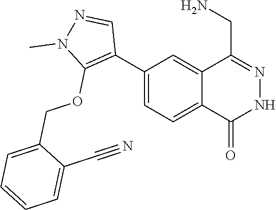 [SMILES: Cn1ncc(-c2ccc3c(=O)[nH]nc(CN)c3c2)c1OCc1ccccc1C#N] | 4B | 4-5 | 2-(((4-(4-(aminomethyl)-1-oxo-1,2- dihydrophthalazin-6-yl)-1-methyl-1H-pyrazol-5- yl)oxy)methyl)benzonitrile LCMS [M + 1]⁺ = 387.2; ¹H NMR (400 MHz, DMSO-d6) δ = 12.88 (s, 1H), 8.57-8.34 (m, 3H), 8.23 (d, J = 8.4 Hz, 1H), 8.07-8.00 (m, 2H), 7.91 (s, 1H), 7.84 (br d, J = 7.6 Hz, 1H), 7.74-7.66 (m, 1H), 7.66-7.61 (m, 1H), 7.54 (br t, J = 7.6 Hz, 1H), 5.22 (s, 2H), 4.46 (br s, 2H), 3.69 (s, 3H) |
| 4-158 | 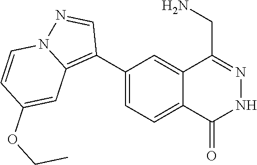 | 4B | 4-6 | 4-(aminomethyl)-6-(5-ethoxypyrazolo[1,5- a]pyridin-3-yl)phthalazin-1(2H)-one LCMS [M + 1]⁺ = 322.2; ¹H NMR (400 MHz, DMSO-d6) δ = 12.84 (s, 1H), 8.68 (d, J = 7.6 Hz, 1H), 8.62 (s, 1H), 8.59 (br s, 3H), 8.34-8.29 (d, J = 8.4 Hz, 1H), 8.27-8.23 (d, J = 8.4 Hz, 1H), 8.11 (s, 1H), 7.42 (d, J = 2.0 Hz, 1H), 6.74 (dd, J = 2.4, 7.6 Hz, 1H), 4.58 (br d, J = 5.6 Hz, 2H), 3.96 (s, 3H) |
| 4-159 | 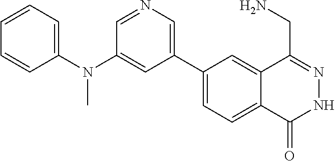 [SMILES: CN(c1ccccc1)c1cncc(-c2ccc3c(=O)[nH]nc(CN)c3c2)c1] | 4A | 4-7 | 4-(aminomethyl)-6-(5- (methyl(phenyl)amino)pyridin-3-yl)phthalazin- 1(2H)-one LCMS [M + 1]⁺ 358.1; ¹H NMR (400 MHz, DMSO-d6) δ = 13.00 (s, 1H), 8.63 (d, J = 1.6 Hz, 1H), 8.43 (br s, 3H), 8.38 (d, J = 8.8 Hz, 1H), 8.27 (d, J = 2.8 Hz, 1H), 8.26-8.19 (m, 2H), 7.84 (t. J = 2.4 Hz. 1H), 7.47-7.38 (m, 2H), 7.24 (dd, J = 0.8, 8.4 Hz, 2H), 7.15 (t, J = 7.2 Hz, 1H), 4.60 (br d, J = 5.6 Hz, 2H), 3.42 (s, 3H) |
| 4-160 | 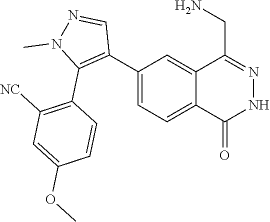 [SMILES: COc1ccc(-c2c(-c3ccc4c(=O)[nH]nc(CN)c4c3)cnn2C)c(C#N)c1] | 4A | 4-1 | 2-(4-(4-(aminomethyl)-1-oxo-1,2- dihydrophthalazin-6-yl)-1-methyl-1H-pyrazol-5- yl)-5-methoxybenzonitrile LCMS [M + 1]⁺ = 387.2; ¹H NMR (400 MHz, DMSO-d6) δ = 12.86 (s, 1H), 8.50-8.35 (br s, 3H), 8.26 (s, 1H), 8.11 (d, J = 8.4 Hz, 1H), 7.76 (s, 1H), 7.70-7.66 (m, 2H), 7.49 (dd, J = 2.8, 8.8 Hz, 1H), 7.44 (d, J = 8.4 Hz, 1H), 4.40-4.22 (m, 2H), 3.92 (s, 3H), 3.72 (s, 3H) |
| 4-161 | 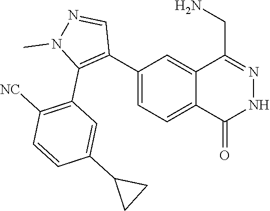 [SMILES: Cn1ncc(-c2ccc3c(=O)[nH]nc(CN)c3c2)c1-c1cc(C2CC2)ccc1C#N] | 4B | 4-1 | 2-(4-(4-(aminomethyl)-1-oxo-1,2- dihydrophthalazin-6-yl)-1-methyl-1H-pyrazol-5- yl)-4-cyclopropylbenzonitrile LCMS [M + 1]⁺ = 397.2; ¹H NMR (400 MHz, DMSO-d6) δ = 12.86 (s, 1H), 8.36 (br s, 3H), 8.27 (s, 1H), 8.10 (d, J = 8.4 Hz, 1H), 7.91 (d, J = 8.0 Hz, 1H), 7.72 (d, J = 1.6 Hz, 1H), 7.48 (s, 1H), 7.47-7.44 (dd, J = 8.4, 2.0 Hz, 1H), 7.43 (dd, J = 1.6, 8.4 Hz, 1H), 4.27 (br s, 2H), 3.72 (s, 3H), 2.15-2.07 (m, 1H), 1.12 (dd, J = 2.8, 8.4 Hz, 2H), 0.95-0.81 (m, 2H) |
| 4-162 | 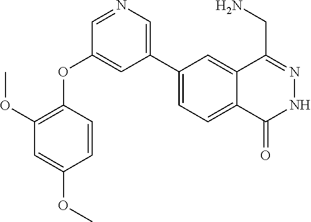 [SMILES: COc1ccc(Oc2cncc(-c3ccc4c(=O)[nH]nc(CN)c4c3)c2)c(OC)c1] | 4A | 4-1 | 4-(aminomethyl)-6-(5-(2,4- dimethoxyphenoxy)pyridin-3-yl)phthalazin- 1(2H)-one LCMS [M + 1]⁺ = 405.1; ¹H NMR (400 MHz, DMSO-d6) δ = 12.99 (s, 1H), 8.84 (d, J = 1.6 Hz, 1H), 8.51 (br s, 3H), 8.37 (d, J = 8.4 Hz, 1H), 8.28 (d, J = 1.2 Hz, 1H), 8.21 (dd, J = 1.6, 8.4 Hz, 1H), 8.19 (d, J = 2.8 Hz, 1H), 7.87 (t, J = 2.0 Hz, 1H), 7.17 (d, J = 8.8 Hz, 1H), 6.79 (d, J = 2.8 Hz, 1H), 6.59 (dd, J = 2.8, 8.8 Hz, 1H), 4.59 (br d, J = 5.6 Hz, 2H), 3.80-3.79 (s, 3H), 3.76 (s, 3H) |
| 4-163 | 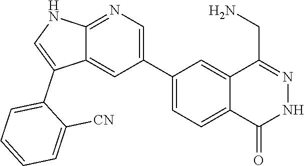 [SMILES: N#Cc1ccccc1-c1c[nH]c2ncc(-c3ccc4c(=O)[nH]nc(CN)c4c3)cc12] | 4B | 4-5 | 2-(5-(4-(aminomethyl)-1-oxo-1,2- dihydrophthalazin-6-yl)-1H-pyrrolo[2,3- b]pyridin-3-yl)benzonitrile LCMS [M + 1]⁺ = 393.2; ¹H NMR (400 MHz, DMSO-d6) δ = 12.95 (s, 1H), 12.46 (d, J = 2.4 Hz, 1H), 8.90 (d, J = 2.0 Hz, 1H), 8.53 (d, J = 2.0 Hz, 1H), 8.48-8.38 (m, 3H), 8.37-8.30 (m, 2H), 8.28 (s, 1H), 8.03 (d, J = 2.8 Hz, 1H), 7.97 (d, J = 6.8 Hz, 1H), 7.91-7.85 (m, 1H), 7.84-7.78 (m, 1H), 7.54 (dt, J = 1.2, 7.6 Hz, 1H), 4.62 (br s, 2H) |
| 4-164 | 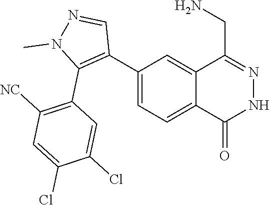 [SMILES: Cn1ncc(-c2ccc3c(=O)[nH]nc(CN)c3c2)c1-c1cc(Cl)c(Cl)cc1C#N] | 4B | 4-5 | 2-(4-(4-(aminomethyl)-1-oxo-1.2- (dihydrophthalazin-6-yl)-1-methyl-1H-pyrazol-5- yl)-4,5-dichlorobenzonitrile LCMS [M + 1]⁺ = 424.9; ¹H NMR (400 MHz, DMSO-d6) δ = 12.87 (s, 1H), 8.52 (br s, 3H), 8.49 (s, 1H), 8.31 (s, 1H), 8.24 (s, 1H), 8.13 (d, J = 8.4 Hz, 1H), 7.91 (d, 4 = 1.2 Hz, 1H), 7.40 (dd, J = 1.6, 8.4 Hz, 1H), 4.39 (br t, J = 5.2 Hz, 2H), 3.78 (s, 3H) |
| 4-165 | 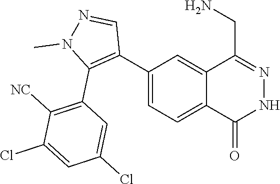 [SMILES: Cn1ncc(-c2ccc3c(=O)[nH]nc(CN)c3c2)c1-c1cc(Cl)cc(Cl)c1C#N] | 4B | 4-4 | 2-(4-(4-(aminomethyl)-1-oxo-1,2- dihydrophthalazin-6-yl)-1-methyl-1H-pyrazol-5- yl)-4,6-dichlorobenzonitrile LCMS [M + 1]⁺ = 425.1; ¹H NMR (400 MHz, DMSO-d6) δ = 12.90 (s, 1H), 8.46-8.31 (br s, 3H), 8.28 (s, 1H), 8.28-8.26 (m, 1H), 8.14 (d, J = 8.4 Hz, 1H), 8.03 (d, J = 2.0 Hz, 1H), 7.84 (d, J = 1.6 Hz, 1H), 7.45 (dd, J = 1.6, 8.4 Hz, 1H), 4.46-4.30 (m, 2H), 3.79 (s, 3H) |
| 4-166 | 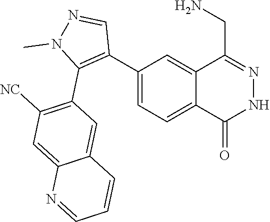 [SMILES: Cn1ncc(-c2ccc3c(=O)[nH]nc(CN)c3c2)c1-c1cc2cccnc2cc1C#N] | 4B | 4-4 | 6-(4-(4-(aminomethyl)-1-oxo-1,2- dihydrophthalazin-6-yl)-1-methyl-1H-pyrazol-5- yl)quinoline-7-carbonitrile LCMS [M + 1]⁺ = 408.2; ¹H NMR (400 MHz, DMSO-d6) δ = 12.84 (s, 1H), 9.18 (dd, J = 1.6, 4.4 Hz, 1H), 8.82 (s, 1H), 8.56 (d, J = 8.8 Hz, 1H), 8.47 (s, 1H), 8.34 (s, 1H), 8.33-8.23 (m, 3H), 8.03 (d, J = 8.4 Hz, 1H), 7.86 (d, J = 1.2 Hz, 1H), 7.83 (dd, J = 4.4, 8.4 Hz, 1H), 7.40 (dd, J = 1.6, 8.4 Hz, 1H), 4.33-4.23 (m, 2H), 3.78 (s, 3H) |
| 4-167 | 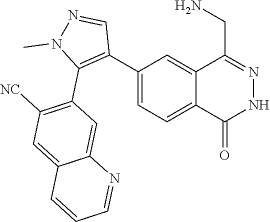 [SMILES: Cn1ncc(-c2ccc3c(=O)[nH]nc(CN)c3c2)c1-c1cc2ncccc2cc1C#N] | 4B | 4-4 | 7-(4-(4-(aminomethyl)-1-oxo-1,2- dihydrophthalazin-6-yl)-1-methyl-1H-pyrazol-5- yl)quinoline-6-carbonitrile LCMS [M + 1]⁺ = 408.2; ¹H NMR (400 MHz, DMSO-d6) δ = 12.84 (s, 1H), 9.17 (dd, J = 1.6, 4.4 Hz, 1H), 8.90 (s, 1H), 8.62 (d, J = 7.6 Hz, 1H), 8.40 (s, 1H), 8.36-8.25 (m, 4H), 8.02 (d, J = 8.4 Hz, 1H), 7.89 (d, J = 1.2 Hz, 1H), 7.83 (dd, J = 4.4, 8.4 Hz, 1H), 7.37 (dd, J = 1.6, 8.4 Hz, 1H), 4.30 (br t, J = 5.6 Hz, 2H), 3.80 (s, 3H) |
| 4-168 | 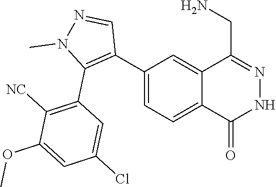 [SMILES: COc1cc(Cl)cc(-c2c(-c3ccc4c(=O)[nH]nc(CN)c4c3)cnn2C)c1C#N] | 4B | 4-5 | 2-(4-(4-(aminomethyl)-1-oxo-1,2- dihydrophthalazin-6-yl)-1-methyl-1H-pyrazol-5- yl)-4-chloro-6-methoxybenzonitrile LCMS [M + 1]⁺ = 421.2; ¹H NMR (400 MHz, DMSO-d6) δ = 12.88 (s, 1H), 8.39 (br s, 3H), 8.26 (s, 1H), 8.13 (d, J = 8.4 Hz, 1H), 7.81 (s, 1H), 7.64 (d, J = 1.6 Hz, 1H), 7.48 (d, J = 1.6 Hz, 1H), 7.44 (d, J = 8.4 Hz, 1H), 4.48-4.25 (m, 2H), 4.01 (s, 3H), 3.75 (s, 3H) |
| 4-169 | 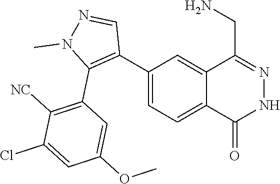 [SMILES: COc1cc(Cl)c(C#N)c(-c2c(-c3ccc4c(=O)[nH]nc(CN)c4c3)cnn2C)c1] | 4B | 4-6 | 2-(4-(4-(aminomethyl)-1-oxo-1,2- dihydrophthalazin-6-yl)-1-methyl-1H-pyrazol-5- yl)-6-chloro-4-methoxybenzonitrile LCMS [M + 1]⁺ = 421.1; ¹H NMR (400 MHz, DMSO-d₆) δ = 12.86 (s, 1H), 8.60 (br s, 3H), 8.32 (s, 1H), 8.12 (d, J = 8.4 Hz, 1H), 7.81 (d, J = 1.2 Hz, 1H), 7.59 (d, J = 2.4 Hz, 1H), 7.47 (dd, J = 1.6, 8.4 Hz, 1H), 7.40 (d, J = 2.4 Hz, 1H), 4.40- 4.22 (m, 2H), 3.96 (s, 3H), 3.76 (s, 3H) |
| 4-170 | 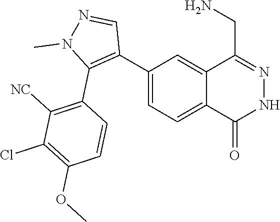 [SMILES: COc1ccc(-c2c(-c3ccc4c(=O)[nH]nc(CN)c4c3)cnn2C)c(C#N)c1Cl] | 4B | 4-4 | 6-(4-(4-(aminomethyl)-1-oxo-1,2- dihydrophthalazin-6-yl)-1-methyl-1H-pyrazol-5- yl)-2-chloro-3-methoxybenzonitrile LCMS [M + 1]⁺ = 421.1; ¹H NMR (400 MHz, DMSO-d6) δ = 12.87 (s, 1H), 8.37 (br s, 3H), 8.27 (s, 1H), 8.11 (d, J = 8.4 Hz, 1H), 7.83 (d, J = 1.2 Hz, 1H), 7.73 (d, J = 8.8 Hz, 1H), 7.70 (d, J = 8.8 Hz, 1H), 7.43 (dd, J = 1.2, 8.4 Hz, 1H), 4.43- 4.29 (m, 2H), 4.04 (s, 3H), 3.73 (s, 3H) |
| 4-171 | 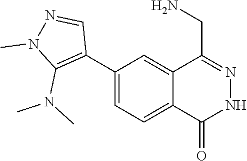 [SMILES: CN(C)c1c(-c2ccc3c(=O)[nH]nc(CN)c3c2)cnn1C] | 4B | 4-8 | 4-(aminomethyl)-6-(5-(dimethylamino)-1- methyl-1H-pyrazol-4-yl)phthalazin-1(2H)-one LCMS [M + 1]⁺ = 299.1; ¹H NMR (400 MHz, DMSO-d6) δ = 12.43 (s, 1H), 8.22 (d, J = 8.4 Hz, 1H), 8.07 (s, 1H), 7.86-8.92 (m, 1H), 7.77 (s, 1H), 4.03 (s, 2H), 3.74 (s, 3H), 2.80 (s, 6H), 2.12- 2.31 (m, 2H) |
| 4-172 | 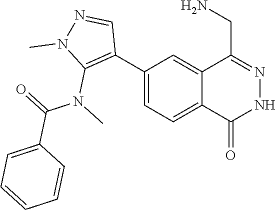 [SMILES: CN(C(=O)c1ccccc1)c1c(-c2ccc3c(=O)[nH]nc(CN)c3c2)cnn1C] | 4B | 4-8 | N-(4-(4-(aminomethyl)-1-oxo-1,2- dihydrophthalazin-6-yl)-1-methyl-1H-pyrazol-5- yl)-N-methylbenzamide LCMS [M + 1]⁺ = 389.2; ¹H NMR (400 MHz, DMSO-d6) δ = 12.47 (s, 1H), 8.17 (d, J = 8.0 Hz, 1H), 7.92 (s, 1H), 7.80 (d, J = 1.2 Hz, 1H), 7.63- 7.72 (m, 1H), 7.19-7.31 (m, 1H), 7.02-7.12 (m, 2H), 6.83-6.93 (m, 2H), 3.99 (s, 2H), 3.83 (s, 3H), 3.45 (s, 3H), 1.80-2.24 (m, 2H). |
| 4-173 | 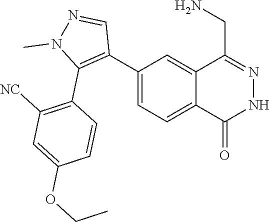 [SMILES: CCOc1ccc(-c2c(-c3ccc4c(=O)[nH]nc(CN)c4c3)cnn2C)c(C#N)c1] | 4B | 4-4 | 2-(4-(4-(aminomethyl)-1-oxo-1,2- dihydrophthalazin-6-yl)-1-methyl-1H-pyrazol-5- yl)-5-ethoxybenzonitrile LCMS [M + 1]⁺ = 401.1; ¹H NMR (400 MHz, DMSO-d6) δ = 12.85 (s, 1H), 8.37 (br s, 3H), 8.25 (s, 1H), 8.10 (d, J = 8.4 Hz, 1H), 7.78 (d, J = 1.2 Hz, 1H), 7.66 (d, J = 8.8 Hz, 1H), 7.64 (d, J = 2.8 Hz, 1H), 7.46 (dd, J = 2.8, 8.8 Hz, 1H), 7.42 (dd, J = 1.6, 8.4 Hz, 1H), 4.39-4.27 (m, 2H), 4.19 (q, J = 6.8 Hz, 2H), 3.71 (s, 3H), 1.38 (t, J = 7.2 Hz, 3H) |
| 4-174 | 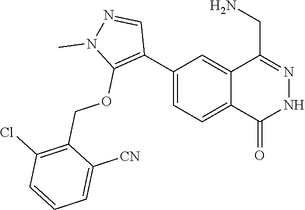 [SMILES: Cn1ncc(-c2ccc3c(=O)[nH]nc(CN)c3c2)c1OCc1c(Cl)cccc1C#N] | 4B | 4-5 | 2-(((4-(4-(aminomethyl)-1-oxo-1,2- dihydrophthalazin-6-yl)-1-methyl-1H-pyrazol-5- yl)oxy)methyl)-3-chlorobenzonitrile LCMS [M + 1]⁺ = 421.2; ¹H NMR (400 MHz, DMSO-d6) δ = 12.87 (s, 1H), 8.44 (br s, 3H), 8.18 (d, J = 8.4 Hz, 1H), 7.94 (s, 1H), 7.89 (dd, J = 1.6, 8.4 Hz, 1H), 7.82 (d, J = 1.2 Hz, 1H), 7.68- 7.63 (m, 1H), 7.63-7.59 (m, 1H), 7.55 (dd, J = 1.6, 7.2 Hz, 1H), 5.23 (s, 2H), 4.46 (s, 2H), 3.75 (s, 3H) |
| 4-175 | 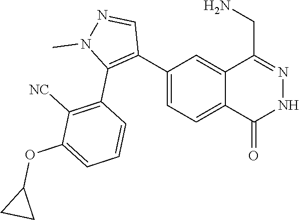 [SMILES: Cn1ncc(-c2ccc3c(=O)[nH]nc(CN)c3c2)c1-c1cccc(OC2CC2)c1C#N] | 4B | 4-4 | 2-(4-(4-(aminomethyl)-1-oxo-1.2- dihydrophthalazin-6-yl)-1-methyl-1H-pyrazol-5- yl)-6-cyclopropoxybenzonitrile LCMS [M + 1]⁺ = 413.0; ¹H NMR (400 MHz, DMSO-d6) δ = 12.87 (s, 1H), 8.41-8.29 (br s, 3H), 8.26 (s, 1H), 8.10 (d, J = 8.4 Hz, 1H), 7.93- 7.88 (m, 1H), 7.75-7.71 (m, 2H), 7.44 (dd, J = 1.6, 8.4 Hz, 1H), 7.32 (d, J = 7.2 Hz, 1H), 4.29 (m, 2H), 4.13 (tt, J = 2.8, 6.4 Hz, 1H), 3.72 (s, 3H), 0.91-0.78 (m, 4H) |
| 4-176 | 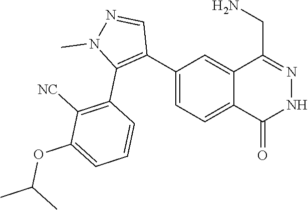 [SMILES: CC(C)Oc1cccc(-c2c(-c3ccc4c(=O)[nH]nc(CN)c4c3)cnn2C)c1C#N] | 4B | 4-4 | 2-(4-(4-(aminomethyl)-1-oxo-1,2- dihydrophthalazin-6-yl)-1-methyl-1H-pyrazol-5- yl)-6-isopropoxybenzonitrile LCMS [M + 1]⁺ = 415.1; ¹H NMR (400 MHz, DMSO-d6) δ = 12.87 (s, 1H), 8.41 (br s, 3H), 8.27 (s, 1H), 8.09 (d, J = 8.4 Hz, 1H), 7.84 (dd, J = 7.6, 8.8 Hz, 1H), 7.79 (d, J = 0.8 Hz, 1H), 7.50 (d, J = 8.8 Hz, 1H), 7.42 (dd, J = 1.2, 8.0 Hz, 1H), 7.26 (d, J = 7.2 Hz, 1H), 4.89 (td, J = 6.0, 12.0 Hz, 1H), 4.39-4.23 (m, 2H), 3.72 (s, 3H), 1.34 (dd, J = 1.2, 6.0 Hz, 6H) |
| 4-177 | 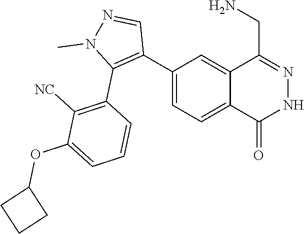 [SMILES: Cn1ncc(-c2ccc3c(=O)[nH]nc(CN)c3c2)c1-c1cccc(OC2CCC2)c1C#N] | 4B | 4-4 | 2-(4-(4-(aminomethyl)-1-oxo-1,2- dihydrophthalazin-6-yl)-1-methyl-1H-pyrazol-5- yl)-6-cyclobutoxybenzonitrile LCMS [M + 1]⁺ = 427.3; ¹H NMR (400 MHz, DMSO-d6) δ = 12.87 (s, 1H), 8.38 (br s, 3H), 8.26 (s, 1H), 8.10 (d, J = 8.4 Hz, 1H), 7.82 (dd, J = 7.6, 8.4 Hz, 1H), 7.75 (s, 1H), 7.43 (d, J = 8.4 Hz, 1H), 7.29 (d, J = 8.8 Hz, 1H), 7.26 (d, J = 7.6 Hz, 1H), 4.93 (t, J = 7.2 Hz, 1H), 4.38-4.21 (m, 2H), 3.72 (s, 3H), 2.55-2.51 (m, 2H), 2.21-2.05 (m, 2H), 1.83 (q, J = 10.2 Hz, 1H), 1.75-1.59 (m, 1H) |
| 4-178 | 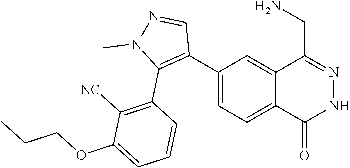 [SMILES: CCCOc1cccc(-c2c(-c3ccc4c(=O)[nH]nc(CN)c4c3)cnn2C)c1C#N] | 4B | 4-4 | 2-(4-(4-(aminomethyl)-1-oxo-1,2- dihydrophthalazin-6-yl)-1-methyl-1H-pyrazol-5- yl)-6-propoxybenzonitrile LCMS [M + 1]⁺ = 415.3; ¹H NMR (400 MHz, DMSO-d6) δ = 12.86 (s, 1H), 8.59 (br s, 3H), 8.31 (s, 1H), 8.09 (d, J = 8.4 Hz, 1H), 7.86 (dd, J = 8.0, 8.4 Hz, 1H), 7.76 (d, J = 1.2 Hz, 1H), 7.48 (d, J = 8.8 Hz, 1H), 7.45 (dd, J = 1.6, 8.4 Hz, 1H), 7.27 (d, J = 7.6 Hz, 1H), 4.30-4.13 (m, 4H), 3.72 (s, 3H), 1.77 (q, J = 7.6 Hz, 2H), 0.99 (t, J = 7.6 Hz, 3H) |
| 4-179 | 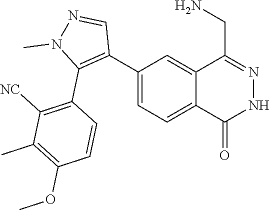 [SMILES: COc1ccc(-c2c(-c3ccc4c(=O)[nH]nc(CN)c4c3)cnn2C)c(C#N)c1C] | 4B | 4-4 | 6-(4-(4-(aminomethyl)-1-oxo-1,2- dihydrophthalazin-6-yl)-1-methyl-1H-pyrazol-5- yl)-3-methoxy-2-methylbenzonitrile LCMS [M + 1]⁺ = 401.2; ¹H NMR (400 MHz, DMSO-d6) δ = 12.85 (s, 1H), 8.35 (br s, 3H), 8.26 (s, 1H), 8.09 (d, J = 8.4 Hz, 1H), 7.78 (s, 1H), 7.54 (d, J = 8.4 Hz, 1H), 7.48 (d, J = 8.4 Hz, 1H), 7.44 (d, J = 9.6 Hz, 1H), 4.37-4.26 (m, 2H), 3.96 (s, 3H), 3.69 (s, 3H), 2.39 (s, 3H) |
| 4-180 | 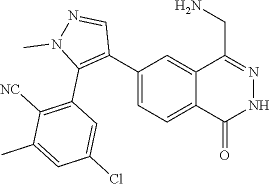 [SMILES: Cc1cc(Cl)cc(-c2c(-c3ccc4c(=O)[nH]nc(CN)c4c3)cnn2C)c1C#N] | 4B | 4-4 | 2-(4-(4-(aminomethyl)-1-oxo-1,2- dihydrophthalazin-6-yl)-1-methyl-1H-pyrazol-5- yl)-4-chloro-6-methylbenzonitrile LCMS [M + 1]⁺ = 405.1; ¹H NMR (400 MHz, DMSO-d6) δ = 12.87 (s, 1H), 8.37 (br s, 3H), 8.28 (s, 1H), 8.12 (d, J = 8.4 Hz, 1H), 7.86 (d, J = 1.6 Hz, 1H), 7.77 (dd, J = 1.6, 7.2 Hz, 2H), 7.46 (dd, J = 1.6, 8.4 Hz, 1H), 4.36-4.26 (m, 2H), 3.74 (s, 3H), 2.54 (s, 3H) |
| 4-181 | 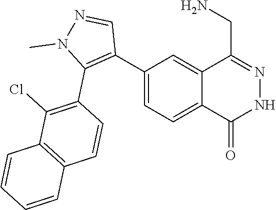 [SMILES: Cn1ncc(-c2ccc3c(=O)[nH]nc(CN)c3c2)c1-c1ccc2ccccc2c1Cl] | 4D | 4-4 | 4-(aminomethyl)-6-(5-(1-chloronaphthalen-2-yl)- 1-methyl-1H-pyrazol-4-yl)phthalazin-1(2H)-one LCMS [M + 1]⁺ = 416.3; ¹H NMR (400 MHz, DMSO- d6) δ = 12.82 (s, 1H), 8.38-8.26 (m, 5H), 8.21- 8.11 (m, 2H), 7.99 (d, J = 8.5 Hz, 1H), 7.89 (d, J = 1.5 Hz, 1H), 7.83-7.77 (m, 2H), 7.60 (d, J = 8.5 Hz, 1H), 7.35 (dd, J = 1.5, 8.0 Hz, 1H), 4.44- 4.23 (m, 2H), 3.67 (s, 3H) |
| 4-182 | 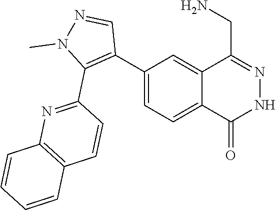 [SMILES: Cn1ncc(-c2ccc3c(=O)[nH]nc(CN)c3c2)c1-c1ccc2ccccc2n1] | 4D | 4-4 | 4-(aminomethyl)-6-(1-methyl-5-(quinolin-2-yl)- 1H-pyrazol-4-yl)phthalazin-1(2H)-one LCMS [M + 1]⁺ = 383.2; ¹H NMR (400 MHz, DMSO- d₆) δ = 12.86 (s, 1H), 8.45 (s, 1H), 8.40 (br s, 3H), 8.16-8.11 (m, 2H), 8.10-8.04 (m, 2H), 7.98 (d, J = 1.2 Hz, 1H), 7.87 (dt, J = 1.2, 7.6 Hz, 1H), 7.76-7.69 (m, 1H), 7.53 (dd, J = 1.2, 8.4 Hz, 1H), 7.47 (d, J = 8.4 Hz, 1H), 4.36 (br d, J = 4.8 Hz, 2H), 3.99 (s, 3H) |
| 4-183 | 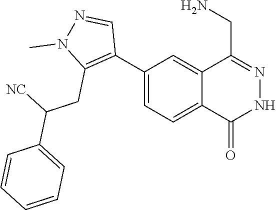 [SMILES: Cn1ncc(-c2ccc3c(=O)[nH]nc(CN)c3c2)c1CC(C#N)c1ccccc1] | 4D | 4-4 | 3-(4-(4-(aminomethyl)-1-oxo-1,2- dihydrophthalazin-6-yl)-1-methyl-1H-pyrazol-5- yl)-2-phenylpropanenitrile LCMS [M + 1]⁺ = 385.1; ¹H NMR (400 MHz, DMSO- d₆) δ = 12.90 (s, 1H), 8.47 (br d, J = 14.0 Hz, 3H), 8.23 (d, J = 8.4 Hz, 1H), 7.85 (d, J = 8.4 Hz, 1H), 7.81 (s, 1H), 7.75 (d, J = 1.2 Hz, 1H), 7.27-7.21 (m, 3H), 7.21-7.14 (m, 2H), 4.65-4.45 (m, 2H), 4.42 (t, J = 7.6 Hz, 1H), 3.82 (s, 3H), 3.76- 3.67 (m, 1H), 3.66-3.57 (m, 1H) |
| 4-184 | 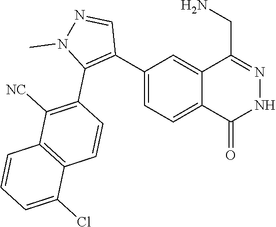 [SMILES: Cn1ncc(-c2ccc3c(=O)[nH]nc(CN)c3c2)c1-c1ccc2c(Cl)cccc2c1C#N] | 4D | 4-6 | 2-(4-(4-(aminomethyl)-1-oxo-1,2- dihydrophthalazin-6-yl)-1-methyl-1H-pyrazol-5- yl)-5-chloro-1-naphthonitrile LCMS [M + 1]⁺ = 441.1; ¹H NMR (400 MHz, DMSO- d₆) δ = 12.84 (s, 1H), 8.70 (d, J = 8.8 Hz, 1H), 8.54-8.39 (m, 3H), 8.38-8.34 (m, 1H), 8.16 (d, J = 8.4 Hz, 1H), 8.04 (d, J = 7.6 Hz, 1H), 8.02- 7.93 (m, 3H), 7.90-7.85 (m, 1H), 7.36-7.24 (m, 1H), 4.34 (br s, 2H), 3.78 (s, 3H) |
| 4-185 | 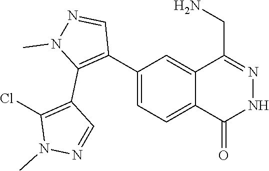 [SMILES: Cn1ncc(-c2c(-c3ccc4c(=O)[nH]nc(CN)c4c3)cnn2C)c1Cl] | 4D | | 4-(aminomethyl)-6-(5′-chloro-1′,2-dimethyl- 1′H,2H-[3,4′-bipyrazol]-4-yl)phthalazin-1(2H)- one LCMS [M + 1]⁺ = 370.0; ¹H NMR (400 MHz, DMSO-d₆) δ = 12.86 (s, 1H), 8.43-8.30 (br s, 3H), 8.19 (s, 1H), 8.16 (d, J = 8.4 Hz, 1H), 7.92 (s, 1H), 7.81 (d, J = 1.2 Hz, 1H), 7.68 (dd, J = 1.6, 8.4 Hz, 1H), 4.40-4.32 (m, 2H), 3.90 (s, 3H), 3.74 (s, 3H) |
| 4-186 | 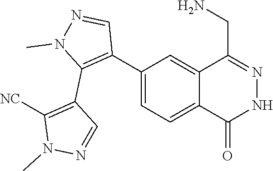 [SMILES: Cn1ncc(-c2c(-c3ccc4c(=O)[nH]nc(CN)c4c3)cnn2C)c1C#N] | 4D | | 4-(4-(aminomethyl)-1-oxo-1,2- dihydrophthalazin-6-yl)-1′,2-dimethyl-1′H,2H- [3,4′-bipyrazole]-5′-carbonitrile LCMS [M + 1] = 361.1; ¹H NMR (400 MHz, DMSO-d₆) δ = 12.89 (s, 1H), 8.40-8.31 (br s, 3H), 8.19-8.17 (m, 2H), 8.10 (s, 1H), 7.83 (d, J = 1.2 Hz, 1H), 7.67 (dd, J = 1.6, 8.4 Hz, 1H), 4.42-4.34 (m, 2H), 4.09 (s, 3H), 3.83 (s, 3H) |
| 4-187 | 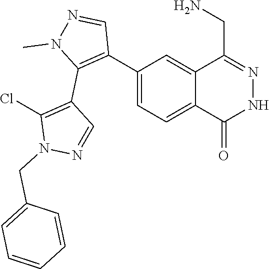 [SMILES: Cn1ncc(-c2ccc3c(=O)[nH]nc(CN)c3c2)c1-c1cnn(Cc2ccccc2)c1Cl] | 4D | | 4-(aminomethyl)-6-(1′-benzyl-5′-chloro-2- methyl-1′H,2H-[3,4′-bipyrazol]-4-yl)phthalazin- 1(2H)-one LCMS [M + 1]⁺ = 446.1; ¹H NMR (400 MHz, DMSO-d₆) δ = 12.86 (s, 1H), 8.50 (br s, 3H), 8.21 (s, 1H), 8.07 (d, J = 8.4 Hz, 1H), 8.05 (s, 1H), 7.89 (d, J = 1.2 Hz, 1H), 7.53 (dd, J = 1.6, 8.4 Hz, 1H), 7.42-7.27 (m, 3H), 7.19-7.11 (m, 2H), 5.48 (s, 2H), 4.44-4.31 (m, 2H), 3.75 (s, 3H |
| 4-188 | 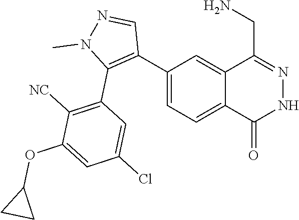 [SMILES: Cn1ncc(-c2ccc3c(=O)[nH]nc(CN)c3c2)c1-c1cc(Cl)cc(OC2CC2)c1C#N] | 4D | | 2-(4-(4-(aminomethyl)-1-oxo-1,2- dibydrophthalazin-6-yl)-1-methyl-1H-pyrazol-5- yl)-4-chloro-6-cyclopropoxybenzonitrile LCMS [M + 1]⁺ = 447.1; ¹H NMR (400 MHz, DMSO-d₆) δ = 12.87 (s, 1H), 8.53 (br s, 3H), 8.28 (s, 1H), 8.12 (d, J = 8.4 Hz, 1H), 7.80 (d, J = 1.8 Hz, 2H), 7.51 (d, J = 1.8 Hz, 1H), 7.44 (dd, J = 1.6, 8.4 Hz, 1H), 4.48-4.26 (m, 2H), 4.21 (td, J = 3.2, 6.0 Hz, 1H), 3.74 (s, 3H), 0.98-0.87 (m, 2H), 0.86-0.73 (m, 2H) |
| 4-189* | 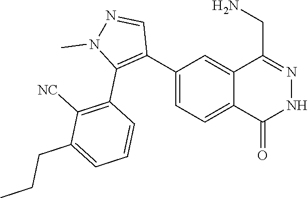 [SMILES: CCCc1cccc(-c2c(-c3ccc4c(=O)[nH]nc(CN)c4c3)cnn2C)c1C#N] | 4D | 4-4 | 2-(4-(4-(aminomethyl)-1-oxo-1,2- dihydrophthalazin-6-yl)-1-methyl-1H-pyrazol-5- yl)-6-propylbenzonitrile LCMS [M + 1]⁺ = 399.2; ¹H NMR (400 MHz, CD₃OD-d₄) δ = 8.19-8.13 (m, 2H), 7.87-7.81 (m, 1H), 7.74 (s, 1H), 7.67 (d, J = 8.0 Hz, 1H), 7.58 (d, J = 7.6 Hz, 1H), 7.51-7.45 (m, 1H), 4.48-4.32 (m, 2H), 3.80 (s, 3H), 2.90-2.83 (m, 2H), 1.74-1.63 (m, 2 H) 0.97-0.91 (m, 3H) |
| 4-190 | 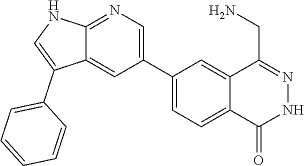 [SMILES: NCc1n[nH]c(=O)c2ccc(-c3cnc4[nH]cc(-c5ccccc5)c4c3)cc12] | 4B | 4-6 | 4-(aminomethyl)-6-(3-phenyl-1H-pyrrolo[2,3- b]pyridin-5-yl)phthalazin-1(2H)-one LCMS [M + 1]⁺ = 368.2; ¹H NMR (400 MHz, DMSO-d₆) δ = 12.94 (s, 1H), 12.19 (br d, J = 7.2 Hz, 1H), 8.85 (s, 1H), 8.71 (s, 1H), 8.57 (br d, J = 1.2 Hz, 3H), 8.38 (s, 2H), 8.31 (br s, 1H), 7.99 (d, J = 2.4 Hz, 1H), 7.84 (d, J = 7.6 Hz, 2H), 7.47 (t, J = 7.6 Hz, 2H), 7.29 (t, J = 7.2 Hz, 1H), 4.63 (br s, 2H) |
| 4-191 | 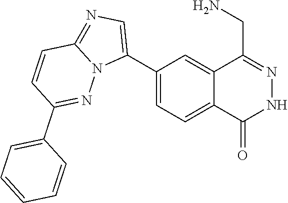 [SMILES: NCc1n[nH]c(=O)c2ccc(-c3cnc4ccc(-c5ccccc5)nn34)cc12] | 4B | 4-4 | 4-(aminomethyl)-6-(6-phenylimidazo[1,2- b]pyridazin-3-yl)phthalazin-1(2H)-one LCMS [M + 1]⁺ = 369.1; ¹H NMR (400 MHz, DMSO-d₆) δ = 12.98 (s, 1H), 8.88 (d, J = 1.2 Hz, 1H), 8.78 (dd, J = 1.6, 8.4 Hz, 1H), 8.67 (s, 1H), 8.50 (br s, 3H), 8.44 (d, J = 8.4 Hz, 1H), 8.41 (d, J = 9.6 Hz, 1H), 8.22 (d, J = 7.2 Hz, 2H), 8.02 (d, J = 9.6 Hz, 1H), 7.71-7.57 (m, 3H), 4.63 (br s, 2H) |
| 4-192 | 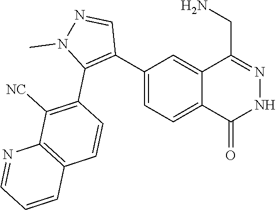 [SMILES: Cn1ncc(-c2ccc3c(=O)[nH]nc(CN)c3c2)c1-c1ccc2cccnc2c1C#N] | 4D | 4-9 | 7-(4-(4-(aminomethyl)-1-oxo-1,2- dihydrophthalazin-6-yl)-1-methyl-1H-pyrazol-5- yl)quinoline-8-carbonitrile LCMS [M + 1]⁺ = 408.2; ¹H NMR (400 MHz, CD₃OD) δ = 9.09 (dd, J = 1.6, 4.0 Hz, 1H), 8.59 (dd, J = 1.6, 8.4 Hz, 1H), 8.47 (d, J = 8.4 Hz, 1H), 8.17 (s, 1H), 8.12 (d, J = 8.4 Hz, 1H), 7.87 (d, J = 8.4 Hz, 1H), 7.84 (s, 1H), 7.79 (dd, J = 4.4, 8.4 Hz, 1H), 7.46 (dd, J = 1.6, 8.4 Hz, 1H), 4.46-4.30 (m, 2H), 3.88 (s, 3H) |
| 4-193 | 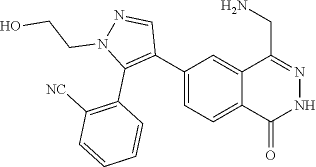 [SMILES: N#Cc1ccccc1-c1c(-c2ccc3c(=O)[nH]nc(CN)c3c2)cnn1CCO] | 4D | 4-2 | 2-(4-(4-(aminomethyl)-1-oxo-1,2- dihydrophthalazin-6-yl)-1-(2-hydroxyethyl)-1H- pyrazol-5-yl)benzonitrile LCMS [M + 1]⁺ = 387.3; ¹H NMR (400 MHz, DMSO-d₆) δ = 12.50-12.33 (br s, 1H), 8.24 (s, 1H), 8.11 (d, J = 8.4 Hz, 1H), 8.04 (d, J = 7.2 Hz, 1H), 7.98-7.92 (m, 1H), 7.85-7.76 (m, 2H), 7.65 (dd, J = 1.6, 8.4 Hz, 1H), 7.54 (s, 1H), 4.98- 4.76 (m, 1H), 4.08-4.01 (br s, 1H), 3.99-3.92 (m, 1H), 3.79-3.73 (m, 1H), 3.70-3.65 (m, 1H), 3.63 (br s, 2H) |
| 4-194 | 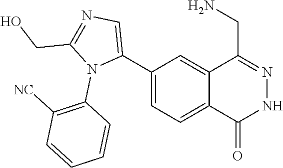 [SMILES: N#Cc1ccccc1-n1c(-c2ccc3c(=O)[nH]nc(CN)c3c2)cnc1CO] | 4D | 4-2 | 2-(5-(4-(aminomethyl)-1-oxo-1,2- dihydrophthalazin-6-yl)-2-(hydroxymethyl)-1H- imidazol-1-yl)benzonitrile LCMS [M + 1]⁺ = 373.3; ¹H NMR (400 MHz, DMSO-d₆) δ = 12.91 (s, 1H), 8.50-8.33 (br s, 3H), 8.13 (d, J = 8.8 Hz, 1H), 8.00-7.93 (m, 3H), 7.75 (ddd, J = 2.8, 6.4, 7.6 Hz, 1H), 7.69 (s, 1H), 7.58-7.50 (m, 2H), 5.37 (t, J = 5.6 Hz, 1H), 4.46 (br d, J = 5.6 Hz, 1H), 4.34 (dd, J = 5.6, 13.2 Hz, 1H), 4.26-4.18 (m, 1H), 4.04 (br d, J = 16.0 Hz, 1H) |
| 4-195 | 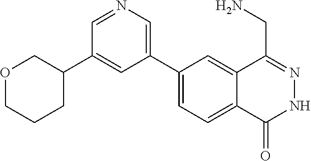 [SMILES: NCc1n[nH]c(=O)c2ccc(-c3cncc(C4CCCOC4)c3)cc12] | 4D | 4-4 | 4-(aminomethyl)-6-(5-(tetrahydro-2H-pyran-3- yl)pyridin-3-yl)phthalazin-1(2H)-one LCMS [M + 1]⁺ = 337.1; ¹H NMR (400 MHz, DMSO-d₆) δ = 13.01 (s, 1H), 9.07 (d, J = 2.0 Hz, 1H), 8.69 (d, J = 2.0 Hz, 1H), 8.49 (br s, 3H), 8.40 (d, J = 2.0 Hz, 1H), 8.34 (t, J = 1.6 Hz, 1H), 8.32 (dd, J = 1.6, 8.0 Hz, 1H), 8.28 (s, 1H), 4.62 (br d, J = 5.6 Hz, 2H), 3.98-3.85 (m, 2H), 3.56- 3.40 (m, 2H), 3.09-2.90 (m, 1H), 2.07-1.86 (m, 2H), 1.74-1.63 (m, 2H) |
| 4-196 | 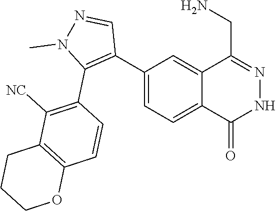 [SMILES: Cn1ncc(-c2ccc3c(=O)[nH]nc(CN)c3c2)c1-c1ccc2c(c1C#N)CCCO2] | 4D | 4-4 | 6-(4-(4-(aminomethyl)-1-oxo-1,2- dihydrophthalazin-6-yl)-1-methyl-1H-pyrazol-5- yl)chromane-5-carbonitrile LCMS [M + 1]⁺ = 435.2; ¹H NMR (400 MHz, DMSO-d₆) δ = 12.85 (s, 1H), 8.39 (br s, 3H), 8.25 (s, 1H), 8.11 (d, J = 8.4 Hz, 1H), 7.72 (d, J = 1.2 Hz, 1H), 7.50 (dd, J = 1.6, 8.4 Hz, 1H), 7.42 (d, J = 8.4 Hz, 1H), 7.28 (d, J = 8.4 Hz, 1H), 4.33- 4.22 (m, 4H), 3.70 (s, 3H), 2.90 (br t, J = 6.4 Hz, 2H), 2.07-1.98 (m, 2H) |
| 4-197 | 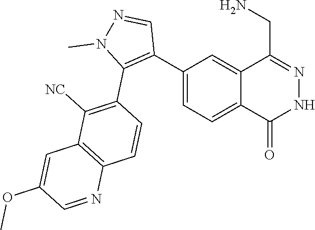 [SMILES: COc1cnc2ccc(-c3c(-c4ccc5c(=O)[nH]nc(CN)c5c4)cnn3C)c(C#N)c2c1] | 4D | 4-4 | 6-(4-(4-(aminomethyl)-1-oxo-1,2- dihydrophthalazin-6-yl)-1-methyl-1H-pyrazol-5- yl)-3-methoxyquinoline-5-carbonitrile LCMS [M + 1]⁺ = 438.2; ¹H NMR (500 MHz, CD₃OD) δ = 8.86 (d, J = 3.0 Hz, 1H), 8.48 (d, J = 8.5 Hz, 1H), 8.19 (s, 1H), 8.15 (d, J = 8.5 Hz, 1H), 7.85 (d, J = 8.5 Hz, 1H), 7.77 (s, 1H), 7.73 (d, J = 2.5 Hz, 1H), 7.55-7.49 (m, 1H), 4.50- 4.20 (m, 2H), 4.05 (s, 3H), 3.87 (s, 3H) |
| 4-198 | 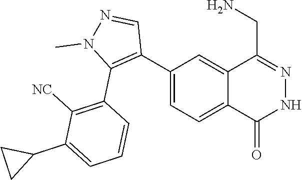 [SMILES: Cn1ncc(-c2ccc3c(=O)[nH]nc(CN)c3c2)c1-c1cccc(C2CC2)c1C#N] | 4E | 4-2 | 2-(4-(4-(aminomethyl)-1-oxo-1,2- dihydrophthalazin-6-yl)-1-methyl-1H-pyrazol-5- yl)-6-cyclopropylbenzonitrile LCMS [M + 1]⁺ = 397.3; ¹H NMR (400 MHz, CD₃OD) δ = 8.23 (d, J = 8.4 Hz, 1H), 8.14 (s, 1H), 7.81-7.75 (m, 1H), 7.70 (d, J = 8.4 Hz, 1H), 7.57 (s, 1H), 7.50-7.45 (m, 1H), 7.30 (d, J = 8.0 Hz, 1H), 3.85 (s, 2H), 3.80 (s, 3H), 2,36- 2.19 (m, 1H), 1.24-1.17 (m, 2H), 0.98-0.86 (m, 2H) |
| 4-199 | 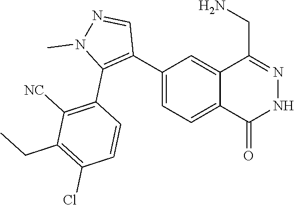 [SMILES: CCc1c(Cl)ccc(-c2c(-c3ccc4c(=O)[nH]nc(CN)c4c3)cnn2C)c1C#N] | 4D | 4-4 4-11 | 6-(4-(4-(aminomethyl)-1-oxo-1,2- dihydrophthalazin-6-yl)-1-methyl-1H-pyrazol-5- yl)-3-chloro-2-ethylbenzonitrile LCMS [M + 1]⁺ = 419.1; ¹H NMR (400 MHz, DMSO-d₆) δ = 12.83 (br s, 1H), 8.28 (s, 1H), 8.10 (d, J = 8.4 Hz, 1H), 8.01 (d, J = 8.0 Hz, 1H), 7.79 (d, J = 1.2 Hz, 1H), 7.66 (d, J = 8.4 Hz, 1H), 7.41 (dd, J = 1.2, 8.4 Hz, 1H), 4.36-4.20 (m, 2H), 3.74 (s, 3H), 2.95 (q, J = 7.6 Hz, 2H), 1.17 (t, J = 7.6 Hz, 3H) |
| 4-200 | 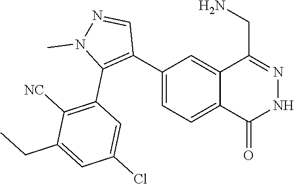 [SMILES: CCc1cc(Cl)cc(-c2c(-c3ccc4c(=O)[nH]nc(CN)c4c3)cnn2C)c1C#N] | 4D | 4-4 | 2-(4-(4-(aminomethyl)-1-oxo-1,2- dihydrophthalazin-6-yl)-1-methyl-1H-pyrazol-5- yl)-4-chloro-6-ethylbenzonitrile LCMS [M + 1]⁺ = 419.2; ¹H NMR (400 MHz, DMSO-d6) δ = 12.88 (s, 1H), 8.38 (br s, 3H), 8.28 (s, 1H), 8.11 (d, J = 8.4 Hz, 1H), 7.85 (d, J = 2.0 Hz, 1H), 7.82 (d, J = 2.0 Hz, 1H), 7.75 (d, J = 1.2 Hz, 1H), 7.42 (dd, J = 1.6, 8.4 Hz, 1H), 4.39- 4.22 (m, 2H), 3.75 (s, 3H), 2.83 (q, J = 7.6 Hz, 2H), 1.21 (t, J = 7.6 Hz, 3H) |
| 4-201 | 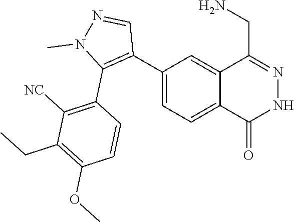 [SMILES: CCc1c(OC)ccc(-c2c(-c3ccc4c(=O)[nH]nc(CN)c4c3)cnn2C)c1C#N] | 4D | 4-4 | 2-(4-(4-(aminomethyl)-1-oxo-1,2- dihydrophthalazin-6-yl)-1-methyl-1H-pyrazol-5- yl)-6-ethyl-5-methoxybenzonitrile LCMS [M + 1]⁺ = 415.3; ¹H NMR (400 MHz, DMSO-d₆) δ = 12.85 (s, 1H), 8.40 (br d, J = 9.6 Hz, 3H), 8,26 (s, 1H), 8.08 (d, J = 8.4 Hz, 1H), 7.77 (s, 1H), 7.62-7.55 (m, 1H), 7.53-7.47 (m, 1H), 7.41 (d, J = 8.4 Hz, 1H), 4.40-4.19 (m, 2H), 3.97 (s, 3H), 3.71 (s, 3H), 2.80(q, J = 7.6 Hz, 2H), 1.12 (t, J = 7.6 Hz, 3H) |
| 4-202 | 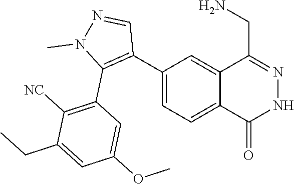 [SMILES: CCc1cc(OC)cc(-c2c(-c3ccc4c(=O)[nH]nc(CN)c4c3)cnn2C)c1C#N] | 4D | 4-1 4-10 | 2-(4-(4-(aminomethyl)-1-oxo-1,2- dibydrophthalazin-6-yl)-1-methyl-1H-pyrazol-5- yl)-6-ethyl-4-methoxybenzonitrile LCMS [M + 1]⁺ = 415.1; ¹H NMR (400 MHz, DMSO-d₆) δ = 12.85 (br s, 1H), 8.47-8.15 (m, 4H), 8.11 (d, J = 8.4 Hz, 1H), 7.73 (d, J = 0.8 Hz, 1H), 7.50 (dd, J = 1.2, 8.4 Hz, 1H), 7.26 (d, J = 2.4 Hz, 1H), 7.20 (d, J = 2.4 Hz, 1H), 4.36-4.16 (m, 2H), 3.92 (s, 3H), 3.74 (s, 3H), 2.80 (q, J = 7.6 Hz, 2H), 1.22 (t, J = 7.6 Hz, 3H) |
| 4-203 | 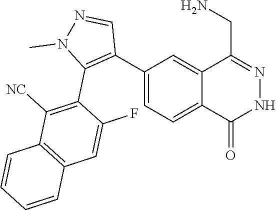 [SMILES: Cn1ncc(-c2ccc3c(=O)[nH]nc(CN)c3c2)c1-c1c(F)cc2ccccc2c1C#N] | 4E | 4-1 | 2-(4-(4-(aminomethyl)-1-oxo-1,2- dibydrophthalazin-6-yl)-1-methyl-1H-pyrazol-5- yl)-3-fluoro-1-naphthonitrile LCMS [M + 1]⁺ = 425.2; ¹H NMR (400 MHz, CD₃OD) δ = 8.28 (d, J = 10.0 Hz, 1H), 8.23 (s, 1H), 8.18 (br dd, J = 3.6, 5.2 Hz, 2H), 8.14 (d, J = 8.4 Hz, 1H), 7.86-7.79 (m, 3H), 7.46 (dd, J = 1.6, 8.0 Hz, 1H), 4.47-4.31 (m, 2H), 3.86 (s, 3H). ¹⁹F NMR (471 MHz, CD₃OD) δ −116.45 (s, 1F) |
| 4-204 | 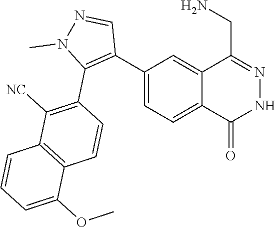 [SMILES: COc1cccc2c(C#N)c(-c3c(-c4ccc5c(=O)[nH]nc(CN)c5c4)cnn3C)ccc12] | 4D | 4-6 | 2-(4-(4-(aminomethyl)-1-oxo-1,2- dihydrophthalazin-6-yl)-1-methyl-1H-pyrazol-5- yl)-5-methoxy-1-naphthonitrile LCMS [M + 1]⁺ = 437.2; ¹H NMR (400 MHz, DMSO-d₆) δ = 12.83 (s, 1H), 8.64 (d, J = 8.8 Hz, 1H), 8.51 (br s, 3H), 8.37 (s, 1H), 7.99 (d, J = 8.4 Hz, 1H), 7.96 (d, J = 1.2 Hz, 1H), 7.84-7.78 (m, 1H), 7.76 (d, J = 8.8 Hz, 1H), 7.69 (d, J = 8.4 Hz, 1H), 7.30 (d, J = 8.0 Hz, 1H), 7.27 (dd, J = 1.2, 8.4 Hz, 1H), 4.34 (br dd, J = 5.6, 10.0 Hz, 2H), 4.07 (s, 3H), 3.76 (s, 3H) |
| 4-205 | 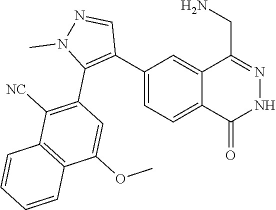 [SMILES: COc1cc(-c2c(-c3ccc4c(=O)[nH]nc(CN)c4c3)cnn2C)c(C#N)c2ccccc12] | 4D | 4-1 | 2-(4-(4-(aminomethyl)-1-oxo-1,2- dihydrophthalazin-6-yl)-1-methyl-1H-pyrazol-5- yl)-4-methoxy-1-naphthonitrile LCMS [M + 1]⁺ = 437.2; ¹H NMR (400 MHz, DMSO-d₆) δ = 12.83 (s, 1H), 8.39 (d, J = 8.4 Hz, 1H), 8.33 (s, 4H), 8.07-8.01 (m, 2H), 7.92 (d, J = 1.2 Hz, 1H), 7.88 (ddd, J = 1.2, 7.2, 8.4 Hz, 1H), 7.80 (dt, J = 1.2, 7.6 Hz, 1H), 7.41 (dd, J = 1.6, 8.4 Hz, 1H), 7.35 (s, 1H), 4.43-4.23 (m, 2H), 4.12 (s, 3H), 3.80 (s, 3H) |
| 4-206 | 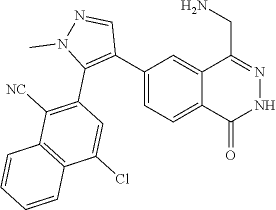 | 4D | 4-6 | 2-(4-(4-(aminomethyl)-1-oxo-1,2- dihydrophthalazin-6-yl)-1-methyl-1H-pyrazol-5- yl)-4-chloro-1-naphthonitrile LCMS [M + 1]⁺ = 444.1; ¹H NMR (400 MHz, DMSO-d₆) δ = 12.84 (s, 1H), 8.57 (br s, 3H), 8.49-8.44 (m, 1H), 8.38 (s, 1H), 8.23-8.19 (m, 1H), 8.16 (s, 1H), 8.04-8.00 (m, 2H), 7.99 (s, 2H), 7.32 (dd, J = 1.6, 8.4 Hz, 1H), 4.45-4.20 (m, 2H), 3.81 (s, 3H) |
| 4-207 | 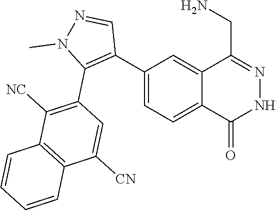 [SMILES: Cn1ncc(-c2ccc3c(=O)[nH]nc(CN)c3c2)c1-c1cc(C#N)c2ccccc2c1C#N] | 4D | 4-4 | 2-(4-(4-(aminomethyl)-1-oxo-1,2- dibydrophthalazin-6-yl)-1-methyl-1H-pyrazol-5~ yl)naphthalene-1,4-dicarbonitrile LCMS [M + 1]⁺ = 432.2; ¹H NMR (400 MHz, DMSO-d₆) δ = 12.84 (s, 1H), 8.64 (s, 1H), 8.53 (br s, 3H), 8.42-8.37 (m, 2H), 8.27 (d, J = 7.6 Hz, 1H), 8.13-8.07 (m, 1H), 8.07-8.02 (m, 1H), 8.00 (d, J = 8.4 Hz, 1H), 7.95 (d, J = 1.6 Hz, 1H), 7.35 (dd, J = 1.6, 8.4 Hz, 1H), 4.42-4.21 (m, 2H), 3.82 (s, 3H) |
| 4-208 | 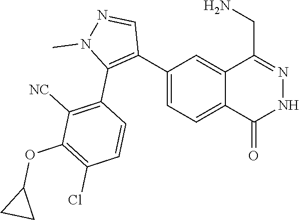 [SMILES: Cn1ncc(-c2ccc3c(=O)[nH]nc(CN)c3c2)c1-c1ccc(Cl)c(OC2CC2)c1C#N] | 4D | 4-1 | 6-(4-(4-(aminomethyl)-1-oxo-1,2- dihydrophthalazin-6-yl)-1-methyl-1H-pyrazol-5- yl)-3-chloro-2-cyclopropoxybenzonitrile LCMS [M + 1]⁺ = 447.0; ¹H NMR (400 MHz, DMSO-d₆) δ = 12.90 (s, 1H), 8.37 (br s, 3H), 8.27 (s, 1H), 8.12 (dd, J = 8.4, 17.6 Hz, 2H), 7.83 (d, J = 0.8 Hz, 1H), 7.59 (d, J = 8.4 Hz, 1H), 7.40 (dd, J = 1.2, 8.0 Hz, 1H), 4.47 (tt, J = 2.8, 6.0 Hz, 1H), 4.43-4.27 (m, 2H), 3.78 (s, 3H), 0.88-0.80 (m, 1H), 0.79-0.70 (m, 1H), 0.65 (td, J = 2.8, 6.0 Hz, 2H) |
| 4-209 | 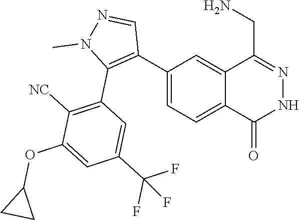 [SMILES: Cn1ncc(-c2ccc3c(=O)[nH]nc(CN)c3c2)c1-c1cc(C(F)(F)F)cc(OC2CC2)c1C#N] | 4D | 4-1 | 2-(4-(4-(aminomethyl)-1-oxo-1,2- dihydrophthalazin-6-yl)-1-methyl-1H-pyrazol-5- yl)-6-cyclopropoxy-4- (trifluoromethyl)benzonitrile LCMS [M + 1]⁺ = 481.2; ¹H NMR (500 MHz, DMSO-d₆) δ = 12.88 (s, 1 H), 8.55 (br s, 3 H), 8.33 (s, 1 H), 8.11 (d, J = 8.4 Hz, 1 H), 7.98 (s, 1 H), 7.84-7.77 (m, 2 H), 7.41 (dd, J = 8.5, 1.5 Hz, 1 H), 4.38-4.29 (m, 3 H), 3.76 (s, 3 H), 0.98-0.91 (m, 2 H), 0.89-0.79 (m, 2 H) |
| 4-210 | 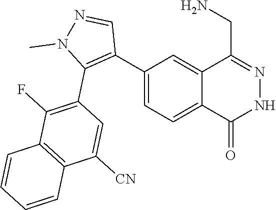 [SMILES: Cn1ncc(-c2ccc3c(=O)[nH]nc(CN)c3c2)c1-c1cc(C#N)c2ccccc2c1F] | 4E | 4-1 | 3-(4-(4-(aminomethyl)-1-oxo-1,2- dihydrophthalazin-6-yl)-1-methyl-1H-pyrazol-5- yl)-4-fluoro-1-naphthonitrile LCMS [M + 1]⁺ = 425.2; ¹H NMR (500 MHz, DMSO-d₆) δ = 12.85 (s, 1H), 8.53 (d, J = 18.5 Hz, 3H), 8.44 (d, J = 6.5 Hz, 1H), 8.35-8.31 (m, 1H), 8.29-8.23 (m, 2H), 8.06-7.97 (m, 3H), 7.90 (t, J = 7.5 Hz, 1H), 7.44 (d, J = 8.0 Hz, 1H), 4.37 (s, 2H), 3.82 (s, 3H) |
| 4-211 | 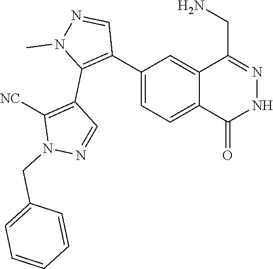 [SMILES: Cn1ncc(-c2ccc3c(=O)[nH]nc(CN)c3c2)c1-c1cnn(Cc2ccccc2)c1C#N] | 4D | 4-6 | 4-(4-(aminomethyl)-1-oxo-1,2- dihydrophthalazin-6-yl)-1′-benzyl-2-methyl- 1′H,2H-[3,4′-bipyrazole]-5′-carbonitrile LCMS [M + 1]⁺ = 437.1; ¹H NMR (400 MHz, DMSO-d₆) δ = 12.89 (s, 1H), 8.48 (br s, 3H), 8.25 (s, 1H), 8.19 (s, 1H), 8.09 (d, J = 8.4 Hz, 1H), 7.89 (d, J = 1.6 Hz, 1H), 7.49 (dd, J = 1.6, 8.4 Hz, 1H), 7.43-7.33 (m, 3H), 7.24-7.20 (m, 2H), 5.62 (s, 2H), 4.41-4.32 (m, 2H), 3.84 (s, 3H) |
| 4-212 | 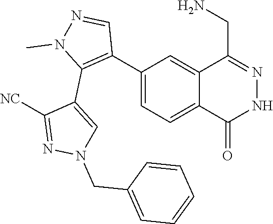 [SMILES: Cn1ncc(-c2ccc3c(=O)[nH]nc(CN)c3c2)c1-c1cn(Cc2ccccc2)nc1C#N] | 4D | 4-6 | 4-(4-(aminomethyl)-1-oxo-1,2- dihydrophthalazin-6-yl)-1′-benzyl-2-methyl- 1′H,2H-[3,4′-bipyrazole]-3′-carbonitrile LCMS [M + 1]⁺ = 437.3; ¹H NMR (400 MHz, DMSO-d₆) δ = 12.89 (s, 1H), 8.63 (s, 1H), 8.48 (br s, 3H), 8.20 (s, 1H), 8.14 (d, J = 8.4 Hz, 1H), 7.89 (d, J = 1.2 Hz, 1H), 7.56 (dd, J = 1.5, 8.4 Hz, 1H), 7.45-7.36 (m, 3H), 7.36-7.30 (m, 2H) 5.58 (s, 2H), 4.38 (br s, 2H), 3.81 (s, 3H) |
| 4-213 | 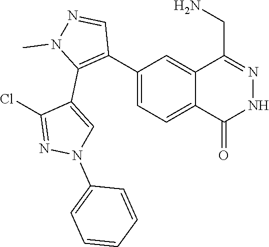 [SMILES: Cn1ncc(-c2ccc3c(=O)[nH]nc(CN)c3c2)c1-c1cn(-c2ccccc2)nc1Cl] | 4D | 4-1 | 4-(aminomethyl)-6-(3′-chloro-2-methyl-1′- phenyl-1′H,2H-[3,4′-bipyrazol]-4-yl)phthalazin- 1(2H)-one LCMS [M + 1]⁺ = 432.2; ¹H NMR (400 MHz, DMSO-d₆) δ = 12.84 (s, 1H), 8.44-8.26 (m, 4H), 7.89 (t, J = 8.0 Hz, 1H), 7.61 (br d, J = 4.8 Hz, 1H), 7.50 (d, J = 8.8 Hz, 1H), 7.40 (br d, J = 2.8 Hz, 1H), 7.32 (d, J = 7.6 Hz, 1H), 4.37-4.27 (m, 2H), 4.25-4.06 (m, 2H), 3.72 (s, 3H), 1.38 (t, J = 7.2 Hz, 3H) |
| 4-214 | 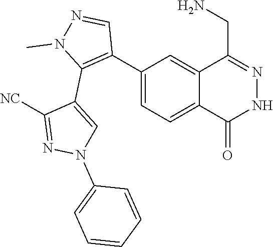 [SMILES: Cn1ncc(-c2ccc3c(=O)[nH]nc(CN)c3c2)c1-c1cn(-c2ccccc2)nc1C#N] | 4D | 4-1 | 4-(4-(aminomethyl)-1-oxo-1,2- dibydrophthalazin-6-yl)-2-methyl-1′-phenyl- 1′H,2H-[3,4′-bipyrazole]-3′-carbonitrile LCMS [M + 1]⁺ = 423.2; ¹H NMR (500 MHz, DMSO-d₆) δ 12.90 (s, 1H), 9.36 (s, 1H), 8.56 (br s, 3H), 8.28 (s, 1H), 8.20-8.17 (m, 1H), 8.03-7.98 (m, 3H), 7.70 (dd, J = 1.6, 8.0 Hz, 1H), 7.64 (t, J = 8.0 Hz, 2H), 7.55-7.50 (m, 1H), 4.41 (q, J = 5.6 Hz, 2H), 3.92 (s, 3H) |
| 4-215 | 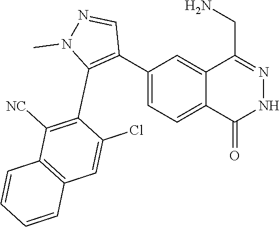 [SMILES: Cn1ncc(-c2ccc3c(=O)[nH]nc(CN)c3c2)c1-c1c(Cl)cc2ccccc2c1C#N] | 4E | 4-6 | 2-(4-(4-(aminomethyl)-1-oxo-1,2- dihydrophthalazin-6-yl)-1-methyl-1H-pyrazol-5- yl)-3-chloro-1-naphthonitrile LCMS [M + 1]⁺ = 441.3; ¹H NMR (400 MHz, CD₃OD) δ = 8.67 (s, 1H), 8.27 (s, 1H), 8.23- 8.18 (m, 2H), 8.15 (d, J = 8.4 Hz, 1H), 7.91- 7.85 (m, 2H), 7.84 (s, 1H), 7.47 (dd, J = 1.6, 8.4 Hz, 1H), 4.55-4.30 (m, 2H), 3.82 (s, 3H) |
| 4-216 | 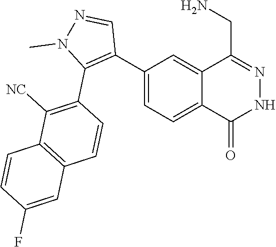 [SMILES: Cn1ncc(-c2ccc3c(=O)[nH]nc(CN)c3c2)c1-c1ccc2cc(F)ccc2c1C#N] | | 4-12 | 2-(4-(4-(aminomethyl)-1-oxo-1,2- dihydrophthalazin-6-yl)-1-methyl-1H-pyrazol-5- yl)-6-fluoro-1-naphthonitrile LCMS [M + 1]⁺ = 425.1; ¹H NMR (400 MHz, DMSO-d₆) δ = 12.37 (br s, 1H), 8.51 (d, J = 8.4 Hz, 1H), 8.26 (s, 1H), 8.20 (dd, J = 5.2, 9.2 Hz. 1H), 8.12 (dd, J = 2.4, 9.2 Hz, 1H), 8.06 (d, J = 8.4 Hz, 1H), 7.89 (d, J = 8.4 Hz, 1H), 7.80 (dt, J = 2.4, 8.8 Hz, 1H), 7.65 (d, J = 1.2 Hz, 1H), 7.60 (dd, J = 1.6, 8.4 Hz, 1H), 3.78 (s, 3H), 3.54 (s, 2H) |
| 4-217 | 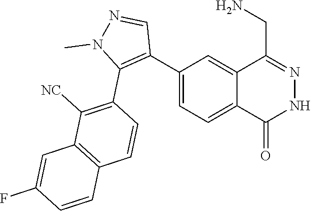 [SMILES: Cn1ncc(-c2ccc3c(=O)[nH]nc(CN)c3c2)c1-c1ccc2ccc(F)cc2c1C#N] | 4D | 4-1 | 2-(4-(4-(aminomethyl)-1-oxo-1,2- dihydrophthalazin-6-yl)-1-methyl-1H-pyrazol-5- yl)-7-fluoro-1-naphthonitrile LCMS [M + 1]⁺ = 425.0; ¹H NMR (400 MHz, DMSO-d₆) δ = 12.83 (s, 1H), 8.58 (d, J = 8.4 Hz, 1H), 8.52-8.38 (m, 4H), 8.36 (s, 1H), 8.01 (d, J = 8.4 Hz, 1H), 7.87 (d, J = 1.2 Hz, 1H), 7.81 (dd, J = 8.8, 17.2 Hz, 3H), 7.35 (dd, J = 1.6, 8.4 Hz, 1H), 4.37-4.17 (m, 2H), 3.78 (s, 3H) |
| 4-218 | 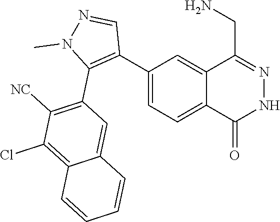 [SMILES: Cn1ncc(-c2ccc3c(=O)[nH]nc(CN)c3c2)c1-c1cc2ccccc2c(Cl)c1C#N] | 4D | 4-1 | 3-(4-(4-(aminomethyl)-1-oxo-1,2- dihydrophthalazin-6-yl)-1-methyl-1H-pyrazol-5- yl)-1-chloro-2-naphthonitrile LCMS [M + 1]⁺ = 441.2; ¹H NMR (400 MHz, DMSO-d₆) δ = 12.85 (s, 1H), 8.47-8.39 (m, 5H), 8.37 (s, 1H), 8.26-8.20 (m, 1H), 8.03 (d, J = 8.4 Hz, 1H), 8.01-7.92 (m, 3H), 7.38 (dd, J = 1.2, 8.4 Hz, 1H), 4.43-4.28 (m, 2H), 3.79 (s, 3H) |
| 4-219 | 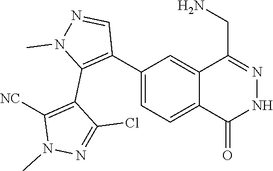 [SMILES: Cn1nc(Cl)c(-c2c(-c3ccc4c(=O)[nH]nc(CN)c4c3)cnn2C)c1C#N] | 4D | 4-6 | 4-(4-(aminomethyl)-1-oxo-1,2- dihydrophthalazin-6-yl)-3′-chloro-1′,2-dimethyl- 1′H,2H-[3,4′-bipyrazole]-5′-carbonitrile LCMS [M + 1]⁺ = 395.2; ¹H NMR (400 MHz, DMSO-d₆) δ = 12.89 (s, 1H), 8.55 (br d, J = 1.6 Hz, 3H), 8.34 (s, 1H), 8.17 (d, J = 8.4 Hz, 1H), 7.87 (d, J = 1.2 Hz, 1H), 7.61 (dd, J = 1.6, 8.0 Hz, 1H), 4.39 (br d, J = 5.2 Hz, 2H), 4.05 (s, 3H), 3.80 (s, 3H) |
| 4-220 | 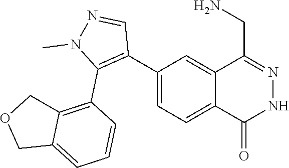 [SMILES: Cn1ncc(-c2ccc3c(=O)[nH]nc(CN)c3c2)c1-c1cccc2c1COC2] | 4D | 4-6 | 4-(aminomethyl)-6-(5-(1,3- dihydroisobenzofuran-4-yl)-1-methyl-1H- pyrazol-4-yl)phthalazin-1(2H)-one LCMS [M + 1]⁺ = 374.1; ¹H NMR (400 MHz, MeOD) δ = ¹H NMR (400 MHz, CDCl₃) δ = 8.21 (m, J = 8.4 Hz, 1H), 8.15 (s, 1H), 7.71 (dd, J = 8.4, 1.2 Hz, 1H), 7.63-7.53 (m, 3H), 7.44 (d, J = 7.2 Hz, 1H), 5.09-5.18 (m, 2H), 4.80 (m, J = 12.4 Hz, 1H), 4.31-4.21 (m, 1H), 4.21-4.14 (m, 1H), 3.76 (s, 3 H); (400 MHz, T = 353K, DMSO- d₆) δ = 12.65 (s, 1H), 8.51 (s, 3H), 8.19 (s, 1H), 8.08 (d, J = 8.4 Hz, 1H), 7.60-7.51 (m, 4H), 7.41- 7.40 (m, 1H), 5.11-5.09 (m, 2H), 4.77-4.74 (m, 1H), 4.40-4.34 (m, 1H), 4.11 (s, 2H), 3.73 (s, 3H) |
| 4-221 | 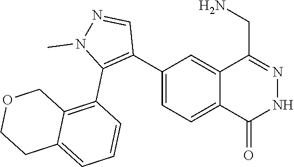 [SMILES: Cn1ncc(-c2ccc3c(=O)[nH]nc(CN)c3c2)c1-c1cccc2c1COCC2] | 4D | 4-6 | 4-(aminomethyl)-6-(5-(isochroman-8-yl)-1- methyl-1H-pyrazol-4-yl)phthalazin-1(2H)-one LCMS [M + 1]⁺ = 388.1; ¹H NMR (400 MHz, MeOD) δ = 8.24-8.19 (m, 2H), 7.77 (dd, J = 1.2, 8.4 Hz, 1H), 7.52 (s, 1H), 7.51-7.43 (m, 2H), 7.28 (d, J = 6.8 Hz, 1H), 4.39-4.33 (m, 1H), 4.23-4.12 (m, 3H), 3.94-3.80 (m, 2H), 3.69 (s, 3H), 3.04-2.89 (m, 2H); (400 MHz, T = 352K, DMSO-d₆) δ = 12.64 (s, 1H), 8.48 (s, 3H), 8.24 (s, 1H), 8.08 (d, J = 8.0 Hz, 1H), 7.61 (d, J = 8.4 Hz, 1H), 7.56 (s, 1H), 7.47-7.39 (m, 2H), 7.27-7.22 (m, 1H), 4.33-4.27 (m, 1H), 4.12- 4.05 (m, 3H), 3.91-3.73 (m, 2H), 3.78 (s, 3H), 3.03-2.84 (m, 2H) |
| 4-222 | 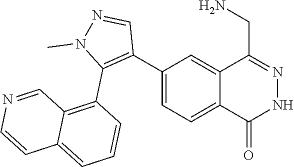 [SMILES: Cn1ncc(-c2ccc3c(=O)[nH]nc(CN)c3c2)c1-c1cccc2ccncc12] | 4D | 4-6 | 4-(aminomethyl)-6-(5-(isoquinolin-8-yl)-1- methyl-1H-pyrazol-4-yl)phthalazin-1(2H)-one LCMS [M + 1]⁺ = 383.1; ¹H NMR (400 MHz, MeOD) δ = 9.20 (s, 1H), 8.68-8.60 (m, 2H), 8.57 (d, J = 8.4 Hz, 1H), 8.44 (t, J = 8.0 Hz, 1H), 8.31 (s, 1H), 8.27 (d, J = 7.2 Hz, 1H), 8.03 (d, J = 8.4 Hz, 1H), 7.77 (s, 1H), 7.39 (d, J = 8.4 Hz, 1H), 4.49-4.39 (m, 1H), 4.31-4.22 (m, 1H), 3.80 (s, 3H) |
| 4-223 | 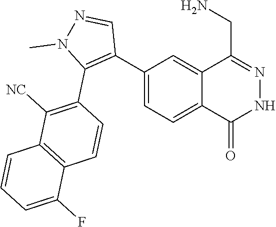 [SMILES: Cn1ncc(-c2ccc3c(=O)[nH]nc(CN)c3c2)c1-c1ccc2c(F)cccc2c1C#N] | 4D | 4-6 | 2-(4-(4-(aminomethyl)-1-oxo-1,2- dihydrophthalazin-6-yl)-1-methyl-1H-pyrazol-5- yl)-5-fluoro-1-naphthonitrile LCMS [M + 1]⁺ = 425.1; ¹H NMR (400 MHz, DMSO-d₆) δ = 12.85 (s, 1H), 8.61 (d, J = 8,4 Hz, 1H), 8.41-8.25 (m, 4H), 8.07-7.96 (m, 2H), 7.96-7.84 (m, 3H), 7.71 (dd, J = 8.0, 10.0 Hz, 1H), 7.36 (dd, J = 1.6, 8.4 Hz, 1H), 4.39-4.20 (m, 2H), 3.80 (s, 3H) |
| 4-224 | 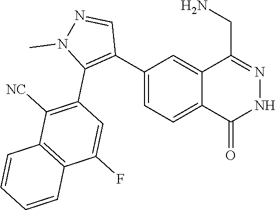 [SMILES: Cn1ncc(-c2ccc3c(=O)[nH]nc(CN)c3c2)c1-c1cc(F)c2ccccc2c1C#N] | 4D | 4-13 | 2-(4-(4-(aminomethyl)-1-oxo-1,2- dihydrophthalazin-6-yl)-1-methyl-1H-pyrazol-5- yl)-4-fluoro-1-naphthonitrile LCMS [M + 1]⁺ = 425.1; ¹H NMR (400 MHz, DMSO-d₆) δ = 12.84 (s, 1H), 8.44 (br s, 3H), 18.37-8.33 (m, 2H), 8.18 (d, J = 8.0 Hz, 1H), 18.04-7.98 (m, 2H), 7.98-7.95 (m, 1H), 7.95- 7.91 (m, 2H), 7.89 (d, J = 10.0 Hz, 1H), 7.73- 7.65 (m, 1H), 7.36 (dd, J = 1.6, 8.4 Hz, 1H), 4.42- 4.27 (m, 2H), 3.80 (s, 3H) |
| 4-225 | 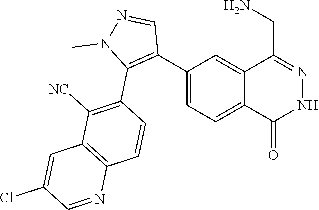 [SMILES: Cn1ncc(-c2ccc3c(=O)[nH]nc(CN)c3c2)c1-c1ccc2ncc(Cl)cc2c1C#N] | 4D | 4-6 | 6-(4-(4-(aminomethyl)-1-oxo-1,2- dihydrophthalazin-6-yl)-1-methyl-1H-pyrazol-5- yl)-3-chloroquinoline-5-carbonitrile LCMS [M + 1]⁺ = 442.1; ¹H NMR (400 MHz, DMSO-d₆) δ = 12.84 (s, 1H), 9.21 (d, J = 2.4 Hz, 1H), 8.57 (d, J = 8.8 Hz, 1H), 8.54-8.52 (m, 1H), 8.44 (br s, 3H), 8.37 (s, 1H), 8.08 (d, J = 8.8 Hz, 1H), 8.02 (d, J = 8.4 Hz, 1H), 7.85 (d, J = 1.2 Hz, 1H), 7.40 (dd, J = 1.6, 8.4 Hz, 1H), 4.34- 4.19 (m, 2H), 3.80 (s, 3H) |
| 4-226 | 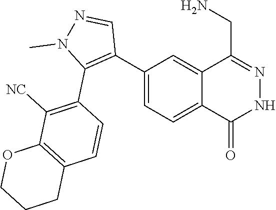 [SMILES: Cn1ncc(-c2ccc3c(=O)[nH]nc(CN)c3c2)c1-c1ccc2c(c1C#N)OCCC2] | 4D | 4-2 | 7-(4-(4-(aminomethyl)-1-oxo-1,2- dihydrophthalazin-6-yl)-1-methyl-1H-pyrazol-5- yl)chromane-8-carbonitrile LCMS [M + 1]⁺ = 413.2; ¹H NMR (400 MHz, DMSO-d₆) δ = 12.40 (br s, 1H), 8.17 (s, 1H), 8.12 (d, J = 8.4 Hz, 1H), 7.70 (dd, J = 1.6, 8.0 Hz, 1H), 7.60 (d, J = 8.0 Hz, 1H), 7.58 (d, J = 1.6) Hz, 1H), 7.14 (d, J = 7.6 Hz, 1H), 4.35 (br t, J = 4.8 Hz, 2H), 3.72 (s, 3H), 3.68 (d, J = 2.0 Hz, 2H), 2.89 (br t, J = 6.0 Hz, 2H), 2.05-2.00 (m, 2H) |
| 4-227 | 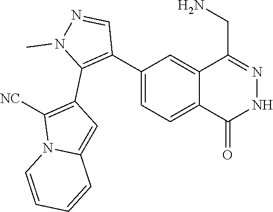 [SMILES: Cn1ncc(-c2ccc3c(=O)[nH]nc(CN)c3c2)c1-c1cc2ccccn2c1C#N] | 4D | 4-6 | 2-(4-(4-(aminomethyl)-1-oxo-1,2- dihydrophthalazin-6-yl)-1-methyl-1H-pyrazol-5- yl)indolizine-3-carbonitrile LCMS [M + 1]⁺ = 396.2; ¹H NMR (400 MHz, DMSO-d6) δ = 12.84 (s, 1H), 8.48-8.45 (m, 1H), 8.41 (br s, 3H), 8.24 (s, 1H), 8.08 (d, J = 8.0 Hz, 1H), 7.94 (d, J = 1.2 Hz, 1H), 7.80 (d, J = 9.2 Hz, 1H), 7.50 (dd, J = 1.6, 8.3 Hz, 1H), 7.30- 7.25 (m, 1H), 7.08 (dt, J = 1.2, 6.8 Hz, 1H), 6.93 (s, 1H), 4.37 (br d, J = 5.2 Hz, 2H), 3.84 (s, 3H) |
| 4-228 | 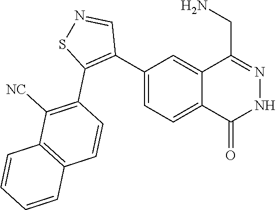 [SMILES: N#Cc1c(-c2sncc2-c2ccc3c(=O)[nH]nc(CN)c3c2)ccc2ccccc12] | 4D | 4-6 | 2-(4-(4-(aminomethyl)-1-oxo-1,2- dibydrophthalazin-6-yl)isothiazol-5-yl)-1- naphthonitrile LCMS [M + 1]⁺ = 409.9; ¹H NMR (400 MHz, DMSO-d₆) δ = 12.96 (s, 1H), 9.21 (s, 1H), 8.49- 8.36 (m, 4H), 8.21 (d, J = 7.6 Hz, 1H), 8.17 (s, 1H), 8.10 (d, J = 8.4 Hz, 2H), 7.90-7.77 (m, 2H), 7.69 (d, J = 8.4 Hz, 1H), 7.52 (dd, J = 1.6, 8.4 Hz, 1H), 4.36 (s, 2H) |
| 4-229 | 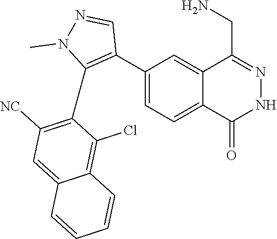 [SMILES: Cn1ncc(-c2ccc3c(=O)[nH]nc(CN)c3c2)c1-c1c(C#N)cc2ccccc2c1Cl] | 4D | 4-6 | 3-(4-(4-(aminomethyl)-1-oxo-1,2- dihydrophthalazin-6-yl)-1-methyl-1H-pyrazol-5- yl)-4-chloro-2-naphthonitrile LCMS [M + 1]⁺ = 441.0/443.0; ¹H NMR (400 MHz, DMSO-d₆) δ = 12.82 (s, 1H), 8.94 (s, 1H), 8.49-8.39 (m, 5H), 8.32 (d, J = 8.0 Hz, 1H), 8.06-8.00 (m, 2H), 7.99-7.93 (m, 2H), 7.27 (d, J = 8.4 Hz, 1H), 4.41-4.32 (m, 2H), 3.71 (s, 3H) |
| 4-230 | 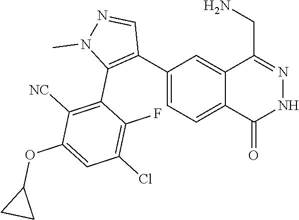 [SMILES: Cn1ncc(-c2ccc3c(=O)[nH]nc(CN)c3c2)c1-c1c(F)c(Cl)cc(OC2CC2)c1C#N] | 4D | 4-6 | 2-(4-(4-(aminomethyl)-1-oxo-1,2- dihydrophthalazin-6-yl)-1-methyl-1H-pyrazol-5- yl)-4-chloro-6-cyclopropoxy-3-fluorobenzonitrile LCMS [M + 1]⁺ = 465.1; ¹H NMR (400 MHz, DMSO-d6) δ = 12.88 (s, 1H), 8.50 (br s, 3H), 8.37 (s, 1H), 8.14 (d, J = 8.4 Hz, 1H), 8.01 (d, J = 6.0 Hz, 1H), 7.85 (s, 1H), 7.47 (d, J = 8.4 Hz, 1H), 4.42-4.30 (m, 2H), 4.22 (br d, J = 3.2 Hz, 1H), 3.78 (s, 3H), 0.96-0.87 (m, 2H), 0.87-0.76 (m, 2H) |
| 4-231 | 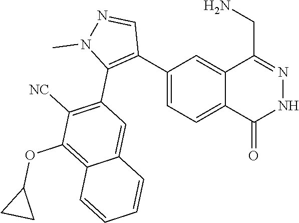 | 4D | 4-6 | 3-(4-(4-(aminomethyl)-1-oxo-1,2- dihydrophthalazin-6-yl)-1-methyl-1H-pyrazol-5- yl)-1-cyclopropoxy-2-naphthonitrile LCMS [M + 1]⁺ = 215.0; ¹H NMR (400 MHz, DMSO-d₆) δ = 12.84 (s, 1H), 8.39 (br s, 3H), 8.35 (s, 1H), 8.29 (d, J = 8.0 Hz, 1H), 8.11 (d. J = 7.6 Hz, 1H), 8.08-8.02 (m, 2H), 7.96 (d, J = 1.2 Hz, 1H), 7.89-7.78 (m, 2H), 7.38 (dd, J = 1.2, 8.4 Hz, 1H), 4.61 (tt, J = 2.8, 6.0 Hz, 1H), 4.42- 4.28 (m, 2H), 3.80 (s, 3H), 1.00-0.90 (m, 2H), 0.80-0.64 (m, 2H) |
| 4-232 | 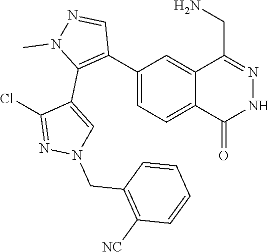 [SMILES: Cn1ncc(-c2ccc3c(=O)[nH]nc(CN)c3c2)c1-c1cn(Cc2ccccc2C#N)nc1Cl] | 4D | 4-6 | 2-((4-(4-(aminomethyl)-1-oxo-1,2- dihydrophthalazin-6-yl)-3′-chloro-2-methyl- 1′H,2H-[3,4′-bipyrazol]-1′-yl)methyl)benzonitrile LCMS [M + 1]⁺ = 471.0; ¹H NMR (400 MHz, DMSO-d₆) δ = 12.86 (s, 1H), 8.43 (s, 4H), 8.21 (s, 1H), 8.12 (d, J = 8.4 Hz, 1H), 7.96-7.87 (m, 2H), 7.77 (dt, J = 1.2, 7.6 Hz, 1H), 7.67-7.55 (m, 2H), 7.49 (d, J = 7.6 Hz, 1H), 5.65 (s, 2H), 4.41 (br s, 2H), 3.74 (s, 3H) |
| 4-233 | 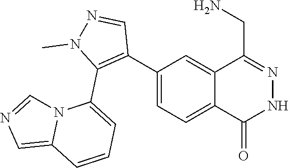 [SMILES: Cn1ncc(-c2ccc3c(=O)[nH]nc(CN)c3c2)c1-c1cccc2cncn12] | 4D | 4-6 | 4-(aminomethyl)-6-(5-(imidazo[1,5-a]pyridin-5- yl)-1-methyl-1H-pyrazol-4-yl)phthalazin-1(2H)- one LCMS [M + 1]⁺ = 372.0; ¹H NMR (400 MHz, DMSO-d₆) δ = 8.72 (s, 1H), 8.39 (s, 1H), 8.07- 7.97 (m, 3H), 7.76 (s, 1H), 7.52-7.38 (m, 2H), 7.35-7.26 (m, 1H), 4.34-4.11 (m, 2H), 3.82 (s, 3H) |
| 4-234 | 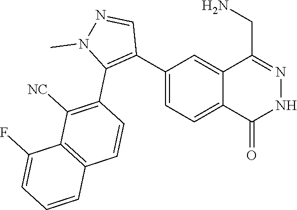 [SMILES: Cn1ncc(-c2ccc3c(=O)[nH]nc(CN)c3c2)c1-c1ccc2cccc(F)c2c1C#N] | 4D | 4-6 | 2-(4-(4-(aminomethyl)-1-oxo-1,2- dihydrophthalazin-6-yl)-1-methyl-1H-pyrazol-5- yl)-8-fluoro-1-naphthonitrile LCMS [M + 1]⁺ = 425.1; ¹H NMR (400 MHz, DMSO-d₆) δ = 12.84 (s, 1H), 8.57 (dd, J = 1.6, 8.4 Hz, 1H), 8.47 (br s, 3H), 8.37 (s, 1H), 8.11 (d, J = 8.0 Hz, 1H), 8.01 (d, J = 8.4 Hz, 1H), 7.90 (d, J = 1.2 Hz, 1H), 7.87-7.78 (m, 2H), 7.67 (dd, J = 7.6, 12.2 Hz, 1H), 7.34 (dd, J = 1.6, 8.4 Hz, 1H), 4.43-4.21 (m, 2H), 3.76 (s, 3H) |
| 4-235 | 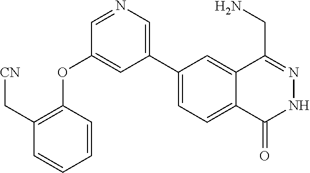 [SMILES: N#CCc1ccccc1Oc1cncc(-c2ccc3c(=O)[nH]nc(CN)c3c2)c1] | 4D | 4-6 | 2-(2-((5-(4-(aminomethyl)-1-oxo-1,2- dihydrophthalazin-6-yl)pyridin-3- yl)oxy)phenyl)acetonitrile LCMS [M + 1]⁺ = 384.0; ¹H NMR (400 MHz, DMSO-d₆) δ = 13.00 (s, 1H), 9.05 (d, J = 1.7 Hz, 1H), 8.60 (br s, 3H), 8.48-8.43 (m, 1H), 8.40- 8.36 (m, 1H), 8.35-8.31 (m, 1H), 8.30-8.22 (m, 1H), 8.19 (t, J = 2.1 Hz, 1H), 7.57 (dd, J = 1.2, 7.6 Hz, 1H), 7.41 (dt, J = 1.5, 7.9 Hz, 1H), 7.34- 7.18 (m, 1H), 7.09-7.01 (m, IH), 4.58 (br d, J = 5.6 Hz, 2H), 4.11 (s, 2H) |
| 4-236 | 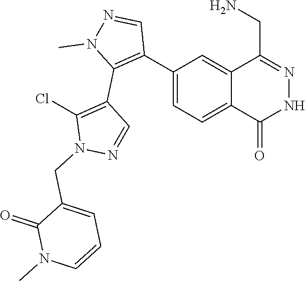 [SMILES: Cn1ncc(-c2ccc3c(=O)[nH]nc(CN)c3c2)c1-c1cnn(Cc2cccn(C)c2=O)c1Cl] | 4D | 4-6 | 4-(aminomethyl)-6-(5′-chloro-2-methyl-1′-((1- methyl-2-oxo-1,2-dihydropyridin-3- yl)methyl)-1′H,2H-[3,4′-bipyrazol]-4- yl)phthalazin-1(2H)-one LCMS [M + 1]⁺ = 477.1; ¹H NMR (400 MHz, DMSO-d₆) δ = 12.85 (s, 1H), 8.43 (s, 3H), 8.28 (s, 1H), 8.20 (s, 1H), 8.14 (d, J = 8.4 Hz, 1 H), 7.85 (d, J = 1.2 Hz, 1 H), 7.75 (d, J = 6.8, 2.0 Hz, 1 H), 7.67 (d, J = 8.4, 1.2 Hz, 1 H), 7.36 (d, J = 6.8, 2.0 Hz, 1 H), 6.28 (t, J = 6.8 Hz, 1 H), 5.20 (s, 2H), 4.41 (d, J = 5.6 Hz, 2 H), 3.73 (s, 3H), 3.45 (s, 3H) |
| 4-237 | 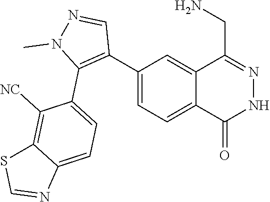 [SMILES: Cn1ncc(-c2ccc3c(=O)[nH]nc(CN)c3c2)c1-c1ccc2ncsc2c1C#N] | 4D | 4-6 | 6-(4-(4-(aminomethyl)-1-oxo-1,2- dihydrophthalazin-6-yl)-1-methyl-1H-pyrazol- 5-yl)benzo[d]thiazole-7-carbonitrile LCMS [M + 1]⁺ = 414.1; ¹H NMR (400 MHz, DMSO-d₆) δ = 12.84 (s, 1H), 9.73 (s, 1H), 8.63 (d, J = 8.4 Hz, 1H), 8.45 (br s, 3H), 8.35 (s, 1H), 8.04 (d, J = 8.4 Hz, 1H), 7.92 (d, J = 8.4 Hz, 1H), 7.87 (s, 1H), 7.36 (dd, J = 1.2, 8.4 Hz, 1H), 4.39-4.19 (m, 2H), 3.78 (s, 3H) |
| 4-238 | 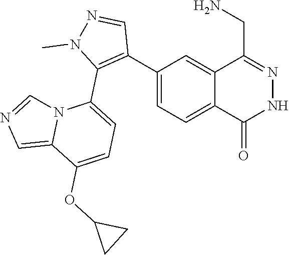 [SMILES: Cn1ncc(-c2ccc3c(=O)[nH]nc(CN)c3c2)c1-c1ccc(OC2CC2)c2cncn12] | 4B | 4-13 | 4-(aminomethyl)-6-(5-(8- cyclopropoxyimidazo[1,5-a]pyridin-5-yl)-1- methyl-1H-pyrazol-4-yl)phthalazin-1(2H)-one LCMS [M + 1]⁺ = 428.0; ¹H NMR (400 MHz, DMSO-d₆) δ = 12.85 (s, 1H), 8.48 (br s, 3H), 8.42-8.37 (m, 1H), 8.15 (br s, 1H), 8.05-7.99 (m, 1H), 7.87 (br s, 1H), 7.72 (br s, 1H), 7.40 (br d, J = 8.4 Hz, 1H), 7.26-7.19 (m, 1H), 6.85- 6.78 (m, 1H), 4.42-4.31 (m. 1H), 4.29-4.17 (m, 2H), 3.81-3.79 (m, 2H), 3.81-3.79 (m, 1H), 2.15-2.07 (m, 1H), 0.99-0.79 (m, 4H) |
| 4-239 | 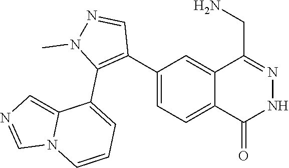 [SMILES: Cn1ncc(-c2ccc3c(=O)[nH]nc(CN)c3c2)c1-c1cccn2cncc12] | 4D | 4-13 | 4-(aminomethyl)-6-(5-(imidazo[1,5-a]pyridin- 8-yl)-1-methyl-1H-pyrazol-4-yl)phthalazin- 1(2H)-one LCMS [M + 1]⁺ = 372.1; ¹H NMR (400 MHz, DMSO-d₆) δ = 12.83 (s, 1H), 9.31 (s, 1H), 8.68 (d, J = 7.2 Hz, 1H), 8.50 (br s, 3H), 8.31 (s, 1H), 8.00 (d, J = 8.4 Hz, 1H), 7.89 (d, J = 1.2 Hz, 1H), 7.48 (dd, J = 1.2, 8.4 Hz, 1H), 7.42 (s, 1H), 7.30 (d, J = 6.8 Hz, 1H), 7.15 (t, J = 6.8 Hz, 1H), 4.40-4.23 (m, 2H), 3.77 (s, 3H) |
| 4-240 | 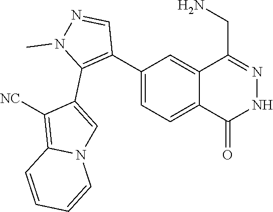 [SMILES: Cn1ncc(-c2ccc3c(=O)[nH]nc(CN)c3c2)c1-c1cn2ccccc2c1C#N] | 4F | 4-6 | 2-(4-(4-(aminomethyl)-1-oxo-1,2- dihydrophthalazin-6-yl)-1-methyl-1H-pyrazol- 5-yl)indolizine-1-carbonitrile LCMS [M + 1]⁺ = 396.1; ¹H NMR (400 MHz, DMSO-d₆) δ = 12.84 (s, 1H), 8.64-8.58 (m, 1H), 8.49 (br s, 3H), 8.27 (s, 1H), 8.11-8.05 (m, 2H), 7.97 (d, J = 1.2 Hz, 1H), 7.70 (d, J = 8.8 Hz, 1H), 7.54 (dd, J = 1.6, 8.0 Hz, 1H), 7.32 (ddd, J = 1.2, 6.8, 9.2 Hz, 1H), 7.04 (dt, J = 1.2, 7.2 Hz, 1H), 4.37 (br d, J = 5.6 Hz, 2H), 3.83 (s, 3H) |
| 4-241 | 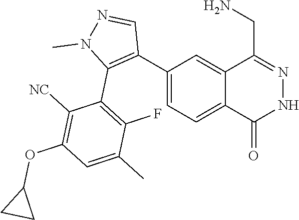 [SMILES: Cc1cc(OC2CC2)c(C#N)c(-c2c(-c3ccc4c(=O)[nH]nc(CN)c4c3)cnn2C)c1F] | 4D | 4-6 | 2-(4-(4-(aminomethyl)-1-oxo-1,2- dihydrophthalazin-6-yl)-1-methyl-1H-pyrazol- 5-yl)-6-cyclopropoxy-3-fluoro-4- methylbenzonitrile LCMS [M + 1]⁺ = 445.2; ¹H NMR (400 MHz, DMSO-d₆) δ = 12.87 (s, 1H), 8.77-8.36 (m, 3H), 8.34 (s, 1H), 8.14 (d, J = 8.4 Hz, 1H), 7.76 (s, 1H), 7.74 (d, J = 6.4 Hz, 1H), 7.49 (d, J = 8.4 Hz, 1H), 4.37-4.24 (m, 2H), 4.11 (td, J = 2.8, 5.6 Hz, 1H), 3.73 (s, 3H), 2.45 (s, 3H), 0.92-0.86 (m, 2H), 0.82-0.74 (m, 2H) |
| 4-242 | 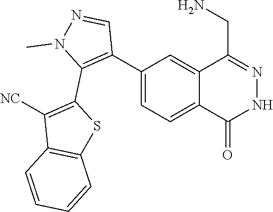 [SMILES: Cn1ncc(-c2ccc3c(=O)[nH]nc(CN)c3c2)c1-c1sc2ccccc2c1C#N] | 4D | 4-8 | 2-(4-(4-(aminomethyl)-1-oxo-1,2- dihydrophthalazin-6-yl)-1-methyl-1H-pyrazol- 5-yl)benzo[b]thiophene-3-carbonitrile LCMS [M + 1]⁺ = 413.1; ¹H NMR (400 MHz, CD₃OD) δ (ppm) = 8.24 (d, J = 8.4 Hz, 1H), 8.15 (s, 1H), 8.13-8.09 (m, 1H), 8.02-7.98 (m, 1H), 7.78-7.75 (m, 1H), 7.72 (dd, J = 1.6, 8.4 Hz, 1H), 7.70-7.61 (m, 2H), 3.98 (s, 3H), 3.83 (s, 2H) |
| 4-243 | 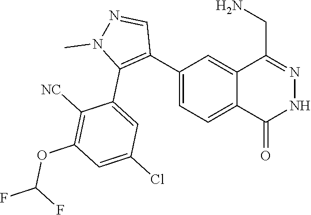 [SMILES: Cn1ncc(-c2ccc3c(=O)[nH]nc(CN)c3c2)c1-c1cc(Cl)cc(OC(F)F)c1C#N] | 4D | 4-6 | 2-(4-(4-(aminomethyl)-1-oxo-1,2- dihydrophthalazin-6-yl)-1-methyl-1H-pyrazol- 5-yl)-4-chloro-6-(difluoromethoxy)benzonitrile LCMS [M]⁺ = 457.1; ¹H NMR (400 MHz, DMSO-d₆) δ (ppm) = 12.89 (s, 1H), 8.55 (br s, 3H), 8.32 (s, 1H), 8.11 (d, J = 8.4 Hz, 1H), 7.90- 7.88 (m, 1H), 7.88-7.85 (m, 1H), 7.83-7.79 (m, 1H), 7.79-7.52 (m, 1H), 7.47-7.42 (m, 1H), 4.39-4.27 (m, 2H), 3.81-3.74 (m, 3H) |
| 4-244 | 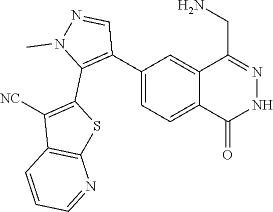 [SMILES: Cn1ncc(-c2ccc3c(=O)[nH]nc(CN)c3c2)c1-c1sc2ncccc2c1C#N] | 4D | 4-8 | 2-(4-(4-(aminomethyl)-1-oxo-1,2- dihydrophthalazin-6-yl)-1-methyl-1H-pyrazol- 5-yl)thieno[2,3-b]pyridine-3-carbonitrile LCMS [M + 1]⁺ = 414.1; ¹H NMR (400 MHz, CD₃OD) δ (ppm) = 8.79 (dd, J = 1.6, 4.8 Hz, 1H), 8.40 (dd, J = 1.6, 8.0 Hz, 1H), 8.26 (d, J = 8.4 Hz, 1H), 8.16 (s, 1H), 7.85 (d, J = 1.2 Hz, 1H), 7.74-7.67 (m, 2H), 4.01 (s, 3H), 3.92 (s, 2H) |
| 4-245 | 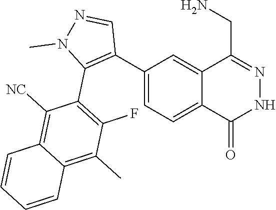 [SMILES: Cc1c(F)c(-c2c(-c3ccc4c(=O)[nH]nc(CN)c4c3)cnn2C)c(C#N)c2ccccc12] | 4G | 4-6 | 2-(4-(4-(aminomethyl)-1-oxo-1,2- dihydrophthalazin-6-yl)-1-methyl-1H-pyrazol- 5-yl)-3-fluoro-4-methyl-1-naphthonitrile LCMS [M + 1]⁺ = 439.2; ¹H NMR (400 MHz, CD₃OD) δ = 8.34 (d, J = 8.0 Hz, 1H), 8.23 (s, 1H), 8.18 (d, J = 8.4 Hz, 1H), 8.12 (d, J = 8.4 Hz, 1H), 7.90-7.83 (m, 2H), 7.83-7.76 (m, 1H), 7.49-7.37 (m, 1H), 4.52-4.29 (m, 2H), 3.85 (s, 3H), 2.79 (d, J = 2.4 Hz, 3H) |
| 4-246 | 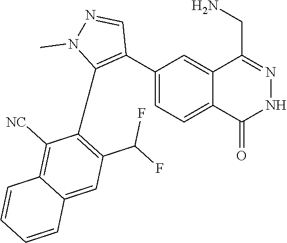 [SMILES: Cn1ncc(-c2ccc3c(=O)[nH]nc(CN)c3c2)c1-c1c(C(F)F)cc2ccccc2c1C#N] | 4D | 4-6 | 2-(4-(4-(aminomethyl)-1-oxo-1,2- dihydrophthalazin-6-yl)-1-methyl-1H-pyrazol- 5-yl)-3-(difluoromethyl)-1-naphthonitrile LCMS [M + 1]⁺ = 457.3; ¹H NMR (400 MHz, DMSO-d₆) δ = 12.70 (br s, 1H), 8.89 (s, 1H), 8.49-8.41 (m, 2H), 8.25 (d, J = 8.1 Hz, 1H), 8.08-7.92 (m, 3H), 7.82 (s, 1H), 7.34 (d, J = 8.4 Hz, 1H), 7.09-6.77 (m, 1H), 4.15-4.04 (m, 2H), 3.66 (s, 3H) |
| 4-247 | 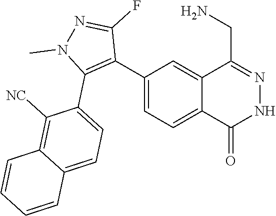 [SMILES: Cn1nc(F)c(-c2ccc3c(=O)[nH]nc(CN)c3c2)c1-c1ccc2ccccc2c1C#N] | 4D | 4-6 | 2-(4-(4-(aminomethyl)-1-oxo-1,2- dihydrophthalazin-6-yl)-3-fluoro-1-methyl-1H- pyrazol-5-yl)-1-naphthonitrile LCMS [M + 1]⁺ = 425.1; ¹H NMR (400 MHz, DMSO-d₆) δ = 12.88 (s, 1H), 8.54 (d, J = 8.4 Hz, 1H), 8.46 (br s, 3H), 8.30-8.24 (m, 1H), 8.13 (d, J = 8.4 Hz, 1H), 8.11 (d, J = 8.4 Hz, 1H), 7.91-7.82 (m, 3H), 7.70 (s, 1H), 7.45 (dd, J = 1.2, 8.4 Hz, 1H), 4.25 (br d, J = 16.0 Hz, 1H), 4.06 (br d, J = 16.0 Hz, 1H), 3.71 (s, 3H) |
| 4-248 | 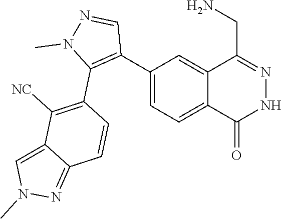 [SMILES: Cn1cc2c(C#N)c(-c3c(-c4ccc5c(=O)[nH]nc(CN)c5c4)cnn3C)ccc2n1] | 4D | 4-6 | 5-(4-(4-(aminomethyl)-1-oxo-1,2- dihydrophthalazin-6-yl)-1-methyl-1H-pyrazol- 5-yl)-2-methyl-2H-indazole-4-carbonitrile LCMS [M + 1]⁺ = 411.2; ¹H NMR (400 MHz, DMSO-d₆) δ = 12.83 (s, 1H), 8.80 (s, 1H), 8.49 (br s, 3H), 8.33 (s, 1H), 8.19 (dd, J = 0.8, 8.8 Hz, 1H), 8.02 (d, J = 8.4 Hz, 1H), 7.86 (d, J = 1.2 Hz, 1H), 7.49 (d, J = 8.8 Hz, 1H), 7.36 (dd, J = 2.4, 8.4 Hz, 1H), 4.33-4.19 (m, 5H), 3.75 (s, 3H) |
| 4-249 | 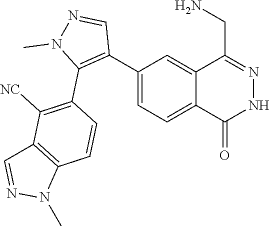 [SMILES: Cn1ncc(-c2ccc3c(=O)[nH]nc(CN)c3c2)c1-c1ccc2c(cnn2C)c1C#N] | 4D | 4-6 | 5-(4-(4-(aminomethyl)-1-oxo-1,2- dihydrophthalazin-6-yl)-1-methyl-1H-pyrazol- 5-yl)-1-methyl-1H-indazole-4-carbonitrile LCMS [M + 1]⁺ = 411.2; ¹H NMR (400 MHz, DMSO-d₆) δ = 12.83 (s, 1H), 8.48-8.37 (m, 3H), 8.36-8.26 (m, 3H), 8.02 (d, J = 8.4 Hz, 1H), 7.83 (s, 1H), 7.72 (d, J = 8.8 Hz, 1H), 7.34 (br d, J = 8.4 Hz, 1H), 4.22 (m, 5H), 3.74 (s, 3H) |
| 4-250 | 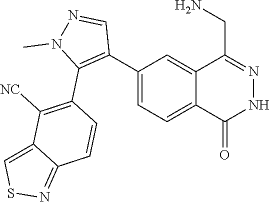 [SMILES: Cn1ncc(-c2ccc3c(=O)[nH]nc(CN)c3c2)c1-c1ccc2nscc2c1C#N] | 4D | 4-6 | 5-(4-(4-(aminomethyl)-1-oxo-1,2- dihydrophthalazin-6-yl)-1-methyl-1H-pyrazol- 5-yl)benzo[c]isothiazole-4-carbonitrile LCMS [M + 1]⁺ = 414.2; ¹H NMR (400 MHz, DMSO-d₆) δ = 12.87 (s, 1H), 10.07 (d, J = 1.2 Hz, 1H), 8.39-8.25 (m, 5H), 8.07 (d, J = 8.4 Hz, 1H), 7.88 (s, 1H), 7.77 (d, J = 9.2 Hz, 1H), 7.45 (dd, J = 1.6, 8.4 Hz, 1H), 4.37-4.23 (m, 2H), 3.84-3.80 (m, 3H) |
| 4-251 | 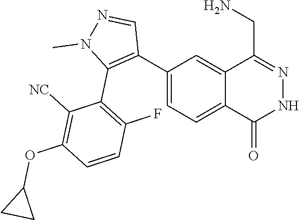 [SMILES: Cn1ncc(-c2ccc3c(=O)[nH]nc(CN)c3c2)c1-c1c(F)ccc(OC2CC2)c1C#N] | 4D | 4-6 | 2-(4-(4-(aminomethyl)-1-oxo-1,2- dihydrophthalazin-6-yl)-1-methyl-1H-pyrazol- 5-yl)-6-cyclopropoxy-3-fluorobenzonitrile LCMS [M + 1]⁺ = 431.1; ¹H NMR (400 MHz, DMSO-d₆) δ = 12.88 (s, 1 H), 8.45 (s, 3 H), 8.35 (s, 1 H), 8.14 (d, J = 8.4 Hz, 1 H), 7.86-7.93 (m, 1 H), 7.77-7.83 (m, 2 H), 7.47 (d, J = 8.4, 1.10 Hz, 1 H), 4.25-4.39 (m, 2 H), 4.12-4.20 (m, 1 H), 3.75 (s, 3 H), 0.85-0.93 (m, 2 H), 0.75-0.84 (m, 2 H) |
| 4-252 | 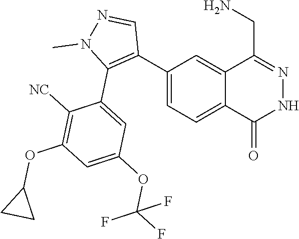 [SMILES: Cn1ncc(-c2ccc3c(=O)[nH]nc(CN)c3c2)c1-c1cc(OC(F)(F)F)cc(OC2CC2)c1C#N] | 4D | 4-6 | 2-(4-(4-(aminomethyl)-1-oxo-1,2- dihydrophthalazin-6-yl)-1-methyl-1H-pyrazol- 5-yl)-6-cyclopropoxy-4- (trifluoromethoxy)benzonitrile LCMS [M + 1]⁺ = 497.1; ¹H NMR (400 MHz, DMSO-d₆) δ = 8.51 (br s, 3H), 8.30 (s, 1H), 8.11 (d, J = 8.4 Hz, 1H), 7.80 (s, 1H), 7.68 (d, J = 1.2 Hz, 1H), 7.46-7.39 (m, 2H), 4.32 (br d, J = 5.6 Hz, 2H), 4.28-4.21 (m, 1H), 3.76 (s, 3H), 0.96-0.77 (m, 4H) |

TABLE 4B

| Example | Structure | CM | PM | Compound Name and Characterization |
|---|---|---|---|---|
| 4-255 | 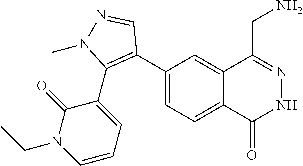 [SMILES: CCn1cccc(-c2c(-c3ccc4c(=O)[nH]nc(CN)c4c3)cnn2C)c1=O] | 4B | 4-14 | 4-(aminomethyl)-6-(5-(1-ethy1-2-oxo-1,2- dihydropyridin-3-yl)-1-methyl-1H-pyrazol- 4-yl)phthalazin-1(2H)-one. LCMS [M + 1]+ = 377.3; ¹H NMR (400 MHz, DMSO-d₆) δ = 12.84 (s, 1H), 8.34 (brs, 3H), 8.16-8.08 (m, 2H), 7.97 (dd, J = 2.0, 6.8 Hz, 1H), 7.83 (s, 1H), 7.63 (dd, J = 1.6, 8.4 Hz, 1H), 7.52 (dd, J = 2.0, 7.2 Hz, 1H), 6.37 (t, J = 6.8 Hz, 1H), 4.37 (br d, J = 4.8 Hz, 2H), 4.08-3.97 (m, 2H), 3.69 (s, 3H), 1.27 (t, J = 7.2 Hz, 3H) |
| 4-256 | 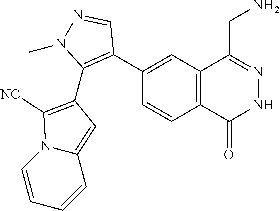 [SMILES: Cn1ncc(-c2ccc3c(=O)[nH]nc(CN)c3c2)c1-c1cc2ccccn2c1C#N] | 4F | 4-6 | 2-[4-[4-(aminomethyl)-1-oxo-2H- phthalazin-6-yl]-2-methyl-pyrazol-3- yl]indolizine-3-carbonitrile. LCMS [M + 1]⁺ = 396.2 ¹H NMR (400 MHz, DMSO-d6) δ = 12.84 (s, 1H), 8.48- 8.45 (m, 1H), 8.41 (br s, 3H), 8.24 (s, 1H), 8.08 (d, J = 8.0 Hz, 1H), 7.94 (d, J = 1.2 Hz, 1H), 7.80 (d, J = 9.2 Hz, 1H), 7.50 (dd, J = 1.6, 8.3 Hz, 1H), 7.30-7.25 (m, 1H), 7.08 (dt, J = 1.2, 6.8 Hz, 1H), 6.93 (s, 1H), 4.37 (br d, J = 5.2 Hz, 2H), 3.84 (s, 3H) |
| 4-257 | 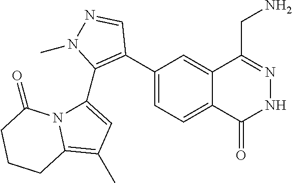 [SMILES: Cc1cc(-c2c(-c3ccc4c(=O)[nH]nc(CN)c4c3)cnn2C)n2c1CCCC2=O] | 4G | 4-6 | 4-(aminomethyl)-6-[1-methyl-5-(1-methyl- 5-oxo-7,8-dihydro-6H-indolizin-3- yl)pyrazol-4-yl]-2H-phthalazin-1-one. LCMS [M + 1]+ = 403.1. ¹H NMR (400 MHz, CD₃OD-d₆) δ = 8.22 (d, J = 9.2 Hz, 1H), 8.09 (s, 1H), 7.69-7.63 (m, 2H), 6.34 (s, 1H), 4.42 (d, J = 1.2 Hz, 2H), 3.70 (s, 3H), 3.00-2.85 (m, 2H), 2.65 (ddd, J = 5.2, 7.6, 12.4 Hz, 2H), 2.13-2.01 (m, 5H). |
| 4-258 | 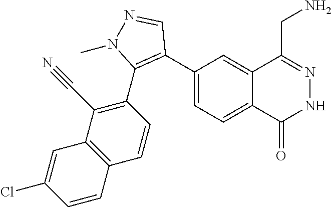 [SMILES: Cn1ncc(-c2ccc3c(=O)[nH]nc(CN)c3c2)c1-c1ccc2ccc(Cl)cc2c1C#N] | 4D | 4-6 | 2-[4-[4-(aminomethyl)-1-oxo-2H- phthalazin-6-yl]-2-methyl-pyrazol-3-yl]-7- chloro-naphthalene-1-carbonitrile. LCMS [M + 1]⁺ = 441.1. ¹H NMR (400 MHz, DMSO-d₆) δ = 12.83 (s, 1H), 8.57 (br d, J = 8.8 Hz, 1H), 8.45 (br s, 3H), 8.37- 8.31 (m, 2H), 8.08 (s, 1H), 8.01 (d, J = 8.4 Hz, 1H), 7.92-7.83 (m, 3H), 7.36 (d, J = 8.4 Hz, 1H), 4.38-4.18 (m, 2H), 3.78 (s, 3H) |
| 4-259 | 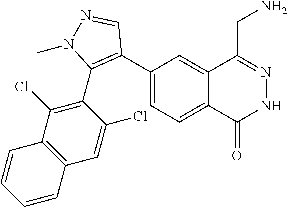 [SMILES: Cn1ncc(-c2ccc3c(=O)[nH]nc(CN)c3c2)c1-c1c(Cl)cc2ccccc2c1Cl] | 4H | 4-15 | 4-(aminomethyl)-6-[5-(1,3-dichloro-2- naphthyl)-1-methyl-pyrazol-4-yl]-2H- phthalazin-1-one. LCMS [M + 1]⁺: 450.0. ¹H NMR (400 MHz, DMSO-d₆) δ = 12.86-12.76 (m, 1H), 8.56- 8.40 (m, 5H), 8.33-8.26 (m, 1H), 8.23-8.15 (m, 1H), 8.02 (d, J = 8.4 Hz, 1H), 7.94 (s, 1H), 7.89-7.80 (m, 2H), 7.32 (d, J = 8.4 Hz, 1H), 4.44-4.26 (m, 2H), 3.65 (s, 3H). |
| 4-260 | 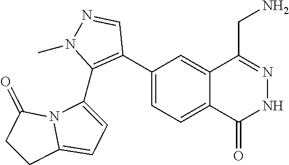 [SMILES: Cn1ncc(-c2ccc3c(=O)[nH]nc(CN)c3c2)c1-c1ccc2n1C(=O)CC2] | 4D | 4-6 | 4-(aminomethyl)-6-[1-methyl-5-(5-oxo-6,7- dihydropyrrolizin-3-yl)pyrazol-4-yl]-2H- phthalazin-1-one. LCMS [M + 1]⁺ = 375.1. ¹H NMR (400 MHz, CD₃OD) δ = 2.89-3.07 (m, 2 H) 3.08- 3.18 (m, 2 H) 3.79 (s, 3 H) 4.43 (s, 2 H) 6.25 (d, J = 3.2 Hz, 1 H) 6.70 (d, J = 3.2 Hz, 1 H) 7.65-7.74 (m, 2 H) 8.11 (s, 1 H) 8.23 (d, J = 8.4 Hz, 1 H) |
| 4-261 | 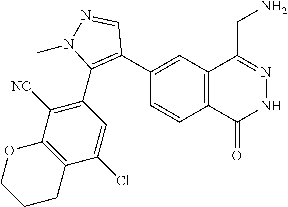 [SMILES: Cn1ncc(-c2ccc3c(=O)[nH]nc(CN)c3c2)c1-c1cc(Cl)c2c(c1C#N)OCCC2] | 4B | 4-6 | 7-[4-[4-(aminomethyl)-1-oxo-2H- phthalazin-6-yl]-2-methyl-pyrazol-3-yl]-5- chloro-chromane-8-carbonitrile. LCMS [M + 1]⁺ = 447.2. ¹H NMR (400 MHz, MeOD-d₄): δ = 8.23 (d, J = 8.4 Hz, 1H), 8.10 (s, 1H), 7.85 (s, 1H), 7.52 (dd, J = 1.6, 8.4 Hz, 1H), 7.31 (s, 1H), 4.57-4.45 (m, 2H), 4.41-4.36 (m, 2H), 3.82 (s, 3H), 2.94 (t, J = 6.4 Hz, 2H), 2.19-2.10 (m, 2H). |
| 4-262 | 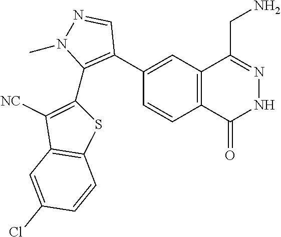 [SMILES: Cn1ncc(-c2ccc3c(=O)[nH]nc(CN)c3c2)c1-c1sc2ccc(Cl)cc2c1C#N] | 4D | 4-6 | 2-[4-[4-(aminomethyl)-1-oxo-2H- phthalazin-6-yl]-2-methyl-pyrazol-3-yl]-5- chloro-benzothiophene-3-carbonitrile. LCMS [M + 1]⁺ = 447.1. 1H NMR (400 MHz, MeOH-d₄) δ = 8.25 (d, J = 8.4 Hz, 1H), 8.16 (s, 1H), 8.12 (d, J = 8.8 Hz, 1H), 7.99 (d, J = 1.6 Hz, 1H), 7.87 (d. J = 1.2 Hz, 1H), 7.64 (ddd, J = 2.0, 3.6, 8.4 Hz, 2H), 4.43 (s, 2H), 4.00 (s, 3H) |
| 4-263 | 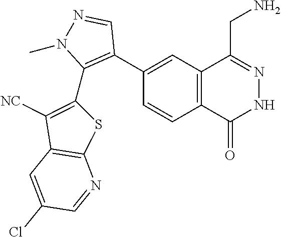 [SMILES: Cn1ncc(-c2ccc3c(=O)[nH]nc(CN)c3c2)c1-c1sc2ncc(Cl)cc2c1C#N] | 4D | 4-6 | 2-[4-[4-(aminomethyl)-1-oxo-2H- phthalazin-6-yl]-2-methyl-pyrazol-3-yl]-5- chloro-thieno[2,3-b]pyridine-3-carbonitrile. LCMS [M + 1]+ = 448.0. ¹H NMR (400 MHz, MeOD-d₄) δ = 8.86 (d, J = 2.0 Hz, 1H), 8.47 (d, J = 8.4 Hz, 1H), 8.21 (d, J = 1.6 Hz, 1H), 8.18 (dd, J = 2.4, 8.4 Hz, 1H), 8.15 (dd, J = 1.6, 8.4 Hz, 1H), 7.48 (d, J = 8.4 Hz, 1H), 4.26 (s, 2H), 2.62 (s, 3H) |
| 4-264 | 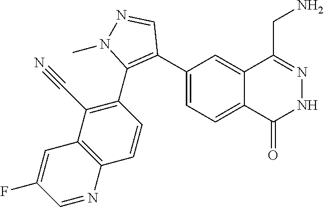 [SMILES: Cn1ncc(-c2ccc3c(=O)[nH]nc(CN)c3c2)c1-c1ccc2ncc(F)cc2c1C#N] | 4D | 4-16 | 6-[4-[4-(aminomethyl)-1-oxo-2H- phthalazin-6-yl]-2-methyl-pyrazol-3-yl]-3- fluoro-quinoline-5-carbonitrile. LCMS [M + 1]: 426.3. ¹H NMR (400 MHz, DMSO-d₆) δ 9.22 (d, J = 2.4 Hz, 1H), 8.58 (d, J = 8.8 Hz, 1H), 8.34 (s, 1H), 8.31-8.22 (m, 2H), 8.05-8.02 (m, 2H), 7.71 (s, 1H), 7.50 (d, J = 8.2 Hz, 1H), 3.99-3.84 (m, 2H), 3.78 (s, 3H) |
| 4-265 | 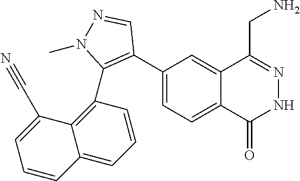 [SMILES: Cn1ncc(-c2ccc3c(=O)[nH]nc(CN)c3c2)c1-c1cccc2cccc(C#N)c12] | 4D | 4-6 | 8-[4-[4-(aminomethyl)-1-oxo-2H- phthalazin-6-yl]-2-methyl-pyrazol-3- yl]naphthalene-1-carbonitrile. LCMS [M + 1]⁺ = 407.2. ¹H NMR (400 MHz, MeOD) δ = 8.43 (dd, J = 1.2, 8.4 Hz. 1H), 8.35 (dd, J = 1.2, 8.4 Hz, 1H), 8.20 (s, 1H), 8.09 (dd, J = 1.2, 7.2 Hz, 1H), 8.04 (d, J = 8.4 Hz, 1H), 7.90-7.84 (m, 1H), 7.83- 7.77 (m, 1H), 7.73 (dd, J = 7.3, 8.2 Hz, 1H), 7.51 (dd, J = 1.5, 8.4 Hz, 1H), 7.48 (s, 1H), 4.18-3.95 (m, 2H), 3.68 (s, 3H) |
| 4-266 | 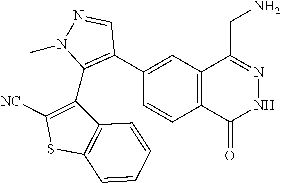 [SMILES: Cn1ncc(-c2ccc3c(=O)[nH]nc(CN)c3c2)c1-c1c(C#N)sc2ccccc12] | 4D | 4-6 | 3-[4-[4-(aminomethyl)-1-oxo-2H- phthalazin-6-yl]-2-methyl-pyrazol-3- yl]benzothiophene-2-carbonitrile. LCMS [M + 1]+ = 413.0. ¹H NMR (400 MHz, MeOD) δ = 8.28 (d, J = 1.6 Hz, 1H), 8.20-8.14 (m, 2H), 7.70 (ddd, J = 1.6, 6.8, 8.4 Hz, 1H), 7.64 (s, 1H), 7.59-7.49 (m, 3H), 4.35-4.06 (m, 2H), 3.84 (s, 3H) |
| 4-267 | 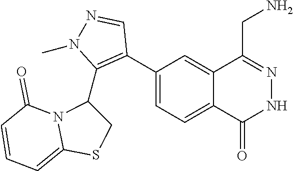 [SMILES: Cn1ncc(-c2ccc3c(=O)[nH]nc(CN)c3c2)c1C1CSc2cccc(=O)n21] | 4D | 4-17 | 3-[4-[4-(aminomethyl)-1-oxo-2H- phthalazin-6-yl]-2-methyl-pyrazol-3-yl] thiazolo[3,2-a]pyridin-5-one. LCMS [ESI, M + 1]: 405.1. ¹H NMR (400 MHz, MeOD) δ 8.54 (s, 1H), 8.18 (d, J = 8.4 Hz, 1H), 8.02 (s, 1H), 7.75-7.65 (m, 2H), 7.63-7.52 (m, 2H), 7.12 (d, J = 7.6 Hz, 1H), 6.34 (d, J = 8.8 Hz, 1H), 4.16 (s, 2H), 3.79 (s, 3H). |
| 4-268 | 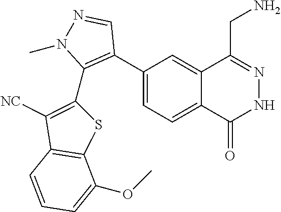 [SMILES: COc1cccc2c(C#N)c(-c3c(-c4ccc5c(=O)[nH]nc(CN)c5c4)cnn3C)sc12] | 4G | 4-13 | 2-[4-[4-(aminomethyl)-1-oxo-2H- phthalazin-6-yl]-2-methyl-pyrazol-3-yl]-7- methoxy-benzothiophene-3-carbonitrile. LCMS [M + 1]⁺ = 443.1. ¹H NMR (400 MHz, DMSO-d₆) δ = 12.90 (s, 1H), 8.37 ( s, 3H), 8.32 (s, 1H), 8.12 (d, J = 8.4 Hz, 1H), 8.00 (s, 1H), 7.65 (d, J = 8.0 Hz, 1H), 7.59- 7.53 (m, 1H), 7.48 (d, J = 8.4 Hz, 1H), 7.26 (d, J = 8.0 Hz, 1H), 4.45-4.36 (m, 2H), 4.02 (s, 3H), 3.93 (s, 3H) |
| 4-269 | 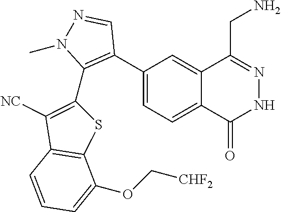 [SMILES: Cn1ncc(-c2ccc3c(=O)[nH]nc(CN)c3c2)c1-c1sc2c(OCC(F)F)cccc2c1C#N] | 4G | 4-13 | 2-(4-(4-(aminomethyl)-1-oxo-1,2- dihydrophthalazin-6-yl)-1-methyl-1H- pyrazol-5-yl)-7-(2,2- difluoroethoxy)benzo[b]thiophene-3- carbonitrile. LCMS [M + 1]⁺ = 493.2. ¹H NMR (400 MHz, DMSO-d₆) δ = 12.89 (s, 1H), 8.47 ( s, 3H), 8.34 (s, 1H), 8.11 (d, J = 8.4 Hz, 1H), 8.04 (s, 1H), 7.70-7.59 (m, 2H), 7.50-7.42 (m, 1H), 7.36 (d, J = 7.6 Hz, 1H), 6.66- 6.31 (m, 1H), 4.64 (dt, J = 3.2, 14.6 Hz, 2H), 4.41 (d, J = 5.2 Hz, 2H), 3.94 (s, 3H). |
| 4-270 | 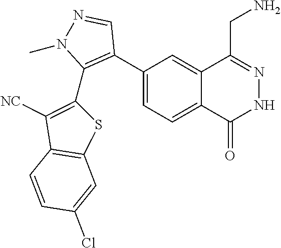 [SMILES: Cn1ncc(-c2ccc3c(=O)[nH]nc(CN)c3c2)c1-c1sc2cc(Cl)ccc2c1C#N] | 4B | 4-13 | 2-(4-(4-(aminomethyl)-1-oxo-1,2- dihydrophthalazin-6-yl)-1-methyl-1H- pyrazol-5-yl)-6-chlorobenzo-[b]thiophene- 3-carbonitrile. LCMS [M + 1]⁺: 447.2. ¹H NMR (400 MHz, CD3OD) δ = 8.24 (d, J = 8.4 Hz, 1H), 8.21 (d, J = 2.0 Hz, 1H), 8.16 (s, 1H), 7.96 (d, J = 8.4 Hz, 1H), 7.89 (s, 1H), 7.69-7.64 (m, 2H), 4.44 (s, 2H), 4.00 (s, 3H). |
| 4-271 | 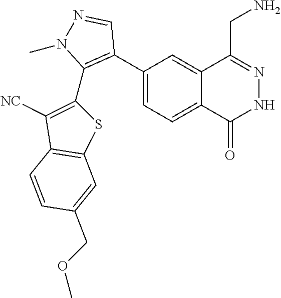 [SMILES: COCc1ccc2c(C#N)c(-c3c(-c4ccc5c(=O)[nH]nc(CN)c5c4)cnn3C)sc2c1] | 4B | 4-13 | 2-(4-(4-(aminomethyl)-1-oxo-1,2- dihydrophthalazin-6-yl)-1-methyl-1H- pyrazol-5-yl)-6- (methoxymethyl)benzo[b]thiophene-3- carbonitrile. LCMS [M + 1]+: 457.4. ¹H NMR (400 MHz, MeOD): δ = 8.23 (d, J = 8.4 Hz, 1H), 8.17 (s, 1H), 7.94 (d, J = 8.0 Hz, 1H), 7,88 (d, J = 1.2 Hz, 1H), 7.66-7.60 (m, 2H), 7.58-7.54 (m, 1H), 4.84 (s, 2H), 4.42 (s, 2H), 3.99 (s, 3H), 3.42 (s, 3H). |
| 4-272 | 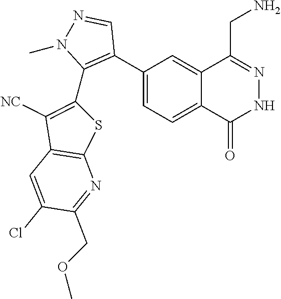 [SMILES: COCc1nc2sc(-c3c(-c4ccc5c(=O)[nH]nc(CN)c5c4)cnn3C)c(C#N)c2cc1Cl] | 4G | 4-6 | 2-(4-(4-(aminomethyl)-1-oxo-1,2- dihydrophthalazin-6-yl)-1-methyl-1H- pyrazol-5-yl)-5-chloro-6- (methoxymethyl)thieno[2,3-b]pyridine-3- carbonitrile. LCMS [M + 1]⁺ = 492.2. ¹H NMR (400 MHz, DMSO-d₆) δ = 12.89 (s, 1H), 8.62 (s, 1H), 8.47 (s, 3H), 8.38 (s, 1H), 8.12 (d, J = 8.4 Hz, 1H), 8.01 (d, J = 1.6 Hz, 1H), 7.57 (dd, J = 1.6, 8.4 Hz, 1H), 4.76 (s, 2H), 4.39 (d, J = 5.2 Hz, 2H), 3.96 (s, 3H), 3.41 (s, 3H). |
| 4-273 | 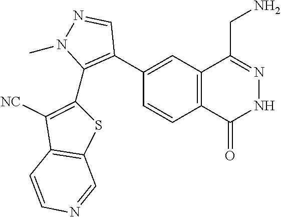 [SMILES: Cn1ncc(-c2ccc3c(=O)[nH]nc(CN)c3c2)c1-c1sc2cnccc2c1C#N] | 4B | 4-13 | 2-[4-[4-(aminomethyl)-1-oxo-2H- phthalazin-6-yl]-2-methyl-pyrazol-3- yl]thieno[2,3-c]pyridine-3-carbonitrile. LCMS [M + 1]⁺ = 414.1. ¹H NMR (400 MHz, DMSO-d₆) δ = 12.90 (s, 1H), 9.61 (s, 1H), 8.77 (d, J = 5.6 Hz, 1H), 8.50 (m, 3H), 8.38 (s, 1H), 8.11 (d, J = 8.4 Hz, 1H), 8.07 (d, J = 5.6 Hz, 1H), 8.01 (s, 1H), 7.53 (d, J = 8.0 Hz, 1H), 4.37 (q, J = 5.6 Hz, 2H), 3.97 (s, 3H). |
| 4-274 | 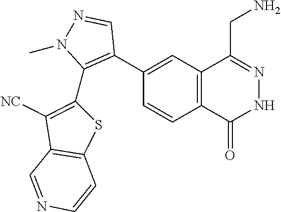 [SMILES: Cn1ncc(-c2ccc3c(=O)[nH]nc(CN)c3c2)c1-c1sc2ccncc2c1C#N] | 4B | 4-13 | 2-[4-[4-(aminomethyl)-1-oxo-2H- phthalazin-6-yl]-2-methyl-pyrazol-3- yl]thieno[3,2-c]pyridine-3-carbonitrile. LCMS [M + 1]⁺ = 413.9. ¹H NMR (400 MHz, DMSO-d₆) δ = 12.89 (s, 1H), 9.30 (s, 1H), 8.72 (d, J = 5.6 Hz, 1H), 8.44 (br s, 3H), 8.38 (d, J = 5.6 Hz, 1H), 8.36 (s, 1H), 8.12 (d, J = 8.4 Hz, 1H), 7.99 (s, 1H), 7.55 (dd, J = 1.6, 8.4 Hz, 1H), 4.37 (br d, J = 5.6 Hz, 2H), 3.96 (s, 3H). |
| 4-275 | 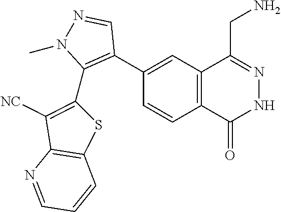 [SMILES: Cn1ncc(-c2ccc3c(=O)[nH]nc(CN)c3c2)c1-c1sc2cccnc2c1C#N] | 4B | 4-13 | 2-(4-(4-(aminomethyl)-1-oxo-1,2- dihydrophthalazin-6-yl)-1-methyl-1H- pyrazol-5-yl)thieno[3,2-b]pyridine-3- carbonitrile. LCMS [M + 1]^{+ =} 414.1.¹H NMR (400 MHz, DMSO-d₆) δ = 12.89 (s, 1H), 8.90 (dd, J = 1.6, 4.8 Hz, 1H), 8.75 (dd, J = 1.6, 8.4 Hz, 1H), 8.44 (m, 3H), 8.36 (s, 1H), 8.12 (d, J = 8.4 Hz, 1H), 8.01 (d, J = 1.2 Hz, 1H), 7.68 (dd, J = 4.4, 8.4 Hz, 1H), 7.53 (dd, J = 1.6, 8.4 Hz, 1H), 4.38 (q, J = 5.2 Hz, 2H), 3.97 (s, 3H). |
| 4-276 | 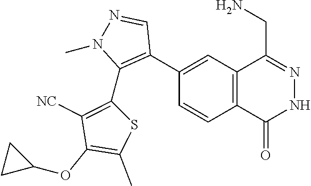 [SMILES: Cc1sc(-c2c(-c3ccc4c(=O)[nH]nc(CN)c4c3)cnn2C)c(C#N)c1OC1CC1] | 4B | 4-6 | 2-(4-(4-(aminomethyl)-1-oxo-1,2- dihydrophthalazin-6-yl)-1-methyl-1H- pyrazol-5-yl)-4-cyclopropoxy-5- methylthiophene-3-carbonitrile. LCMS [M + 1]⁺: 433.2. ¹H NMR (400 MHz, CD₃OD) δ = 8.30 (d, J = 8.4 Hz, 1H), 8.10 (s, 1H), 7.84 (d, J = 1.2 Hz, 1H), 7,62 (dd, J = 1.6, 8,4 Hz, 1H), 4.51 (s, 2H), 4.19 (tt, J = 2.8, 6.0 Hz, 1H), 3.93 (s, 3H), 2.45 (s, 3H), 0.88-0.81 (m, 2H), 0.73-0.65 (m, 2H). |
| 4-277 | 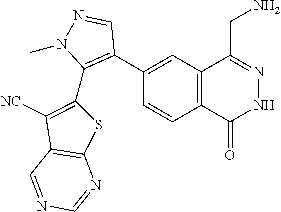 [SMILES: Cn1ncc(-c2ccc3c(=O)[nH]nc(CN)c3c2)c1-c1sc2ncncc2c1C#N] | — | — | 6-(4-(4-(aminomethyl)-1-oxo-1,2- dihydrophthalazin-6-yl)-1-methyl-1H- pyrazol-5-yl)thieno[2,3-d]pyrimidine-5- carbonitrile LCMS [M + 1]⁺ 415.1; ¹H NMR (400 MHz, MeOD-d₄) δ = 9.41 (s, 1H), 9.28 (s, 1H), 8.26 (d, J = 8.4 Hz, 1H), 8.17 (s, 1H), 7.94 (s, 1H), 7.65 (d, J = 8.4 Hz, 1H), 7.51-7.32 (m, 2H), 4.50 (s, 2H), 4.03 (s, 3H) |
| 4-278 | 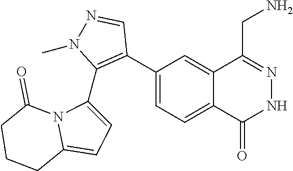 [SMILES: Cn1ncc(-c2ccc3c(=O)[nH]nc(CN)c3c2)c1-c1ccc2n1C(=O)CCC2] | — | — | 4-(aminomethyl)-6-(1-methyl-5-(5-oxo- 5,6,7,8-tetrahydroindolizin-3-yl)-1H- pyrazol-4-yl)phthalazin-1(2H)-one LCMS [M + 1]⁺ 389.2; ¹H NMR (400 MHz, DMSO-d₆) δ = 12.78 (s, 1H), 11.85 (br s, 1H), 8.44 (br s, 3H), 8.11-8.02 (m, 2H), 7.70 (dd, J = 2.8, 4.0 Hz, 2H), 6.88 (d, J = 2.4 Hz, 1H), 4.33-4.22 (m, 2H), 3.59 (s, 3H), 2.97-2.85 (m, 2H), 2.35-2.31 (m, 2H), 2.11-2.05 (m, 2H). |
| 4-279 | 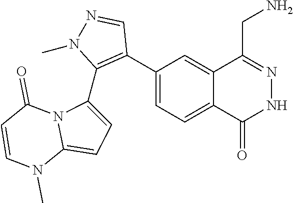 [SMILES: Cn1ncc(-c2ccc3c(=O)[nH]nc(CN)c3c2)c1-c1ccc2n(C)ccc(=O)n12] | 4G | 4-6 | 4-(aminomethyl)-6-[1-methyl-5-(1-methyl- 4-oxo-pyrrolo[1,2-a]pyrimidin-6-yl)pyrazol- 4-yl]-2H-phthalazin-1-one. LCMS [M + 1]⁺: 402.1. ¹H NMR f (400 MHz, DMSO-d₆) δ = 12.77 (s, 1H), 8.58 (br s, 3H), 8.17 (s, 1H), 8.03 (d, J = 8.4 Hz, 1H), 7.86-7.80 (m, 1H), 7.56 (d, J = 1.2 Hz, 1H), 7.51 (dd, J = 1.6, 8.4 Hz, 1H), 6.80- 6.75 (m, 1H), 6.29 (d, J = 4.0 Hz, 1H), 5.42 (d, J = 7.6 Hz, 1H), 4.24-4.10 (m, 2H), 3.76 (s, 3H), 3.62-3.58 (m, 3H). |
| 4-280 | 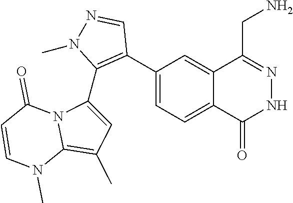 [SMILES: Cc1cc(-c2c(-c3ccc4c(=O)[nH]nc(CN)c4c3)cnn2C)n2c(=O)ccn(C)c12] | 4G | 4-13 | 4-(aminomethyl)-6-[5-(1,8-dimethyl-4-oxo- pyrrolo[1,2-a] pyrimidin-6-yl)-1-methyl-pyrazol-4-yl]-2H- phthalazin-1-one. LCMS [M + 1]⁺: 416.0. ¹H NMR (400 MHz, DMSO-d₆) δ = 12.79 (s, 1H), 8.41 (br s, 3H), 8.12 (s, 1H), 8.05 (d, J = 8.4 Hz, 1H), 7.69 (d, J = 7.6 Hz, 1H), 7.56 (d, J = 1.2 Hz, 1H), 7.50 (dd, J = 1.6, 8.4 Hz, 1H), 6.57 (s, 1H), 5.31 (d, J = 7.6 Hz, 1H), 4.30-4.14 (m, 2H), 3.97 (s, 3H), 3.55 (s, 3H), 3.17 (s, 3H). |
| 4-281 | 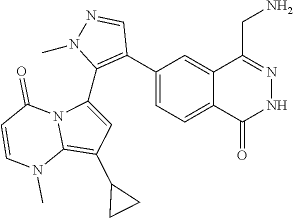 [SMILES: Cn1ncc(-c2ccc3c(=O)[nH]nc(CN)c3c2)c1-c1cc(C2CC2)c2n(C)ccc(=O)n12] | 4G | 4-6 | 4-(aminomethyl)-6-[5-(8-cyclopropy1-1- methyl-4-oxo- pyrrolo[1,2-a]pyrimidin-6-yl)-1-methyl- pyrazol-4-yl]-2H-phthalazin-1-one. LCMS [M + 1]⁺: 442.4. ¹H NMR (400 MHz, DMSO-d₆) δ = 12.78 (s, 1H), 8.61 (br s, 3H), 8.14 (s, 1H), 8.04 (d, J = 8.4 Hz, 1H), 7.74 (d, J = 7.4 Hz, 1H), 7.51 (d, J = 1.6 Hz, 1H), 7.45 (dd, J = 1.6, 8.4 Hz, 1H), 6.51 (s, 1H), 5.34 (d, J = 7.6 Hz, 1H), 4.31-4.16 (m, 2H), 4.15 (s, 3H), 3.54 (s, 3H), 2.29 (ddd, J = 3.2, 5.4, 8.4 Hz, 1H), 0.86 (qd, J = 2.4, 8.4 Hz, 2H), 0.76-0.69 (m, 1H), 0.68- 0.61 (m, 1H). |
| 4-282 | 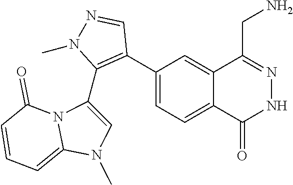 [SMILES: Cn1ncc(-c2ccc3c(=O)[nH]nc(CN)c3c2)c1-c1cn(C)c2cccc(=O)n12] | 4D | 4-6 | 4-(aminomethyl)-6-[1-methyl-5-(1-methyl- 5-oxo-imidazo[1,2-a]pyridin-3-yl)pyrazol- 4-yl]-2H-phthalazin-1-one. LCMS [M + 1]⁺ = 402.2. ¹H NMR (400 MHz, CD₃OD) δ = 8.21 (d, J = 8.4 Hz, 1H), 8.14-8.02 (m, 1H), 7.98-7.73 (m, 4H), 7.67 (br d, J = 8.4 Hz, 1H), 7.18-6.90 (m, 1H), 4.39 (s, 2H), 3.96-3.91 (m, 3H), 3.80 (s, 3H). |
| 4-283 | 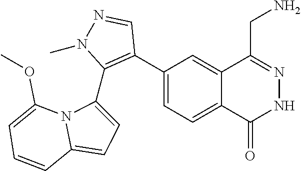 [SMILES: COc1cccc2ccc(-c3c(-c4ccc5c(=O)[nH]nc(CN)c5c4)cnn3C)n12] | 4D | 4-6 | 4-(aminomethyl)-6-[5-(5-methoxyindolizin- 3-yl)-1-methyl-pyrazol-4-yl]-2H-phthalazin- 1-one. LCMS [M + 1]⁺: 401.2. ¹H NMR (400 MHz, DMSO-d₆) δ = 12.77 (s, 1H), 8.54 (br s, 3H), 8.23 (s, 1H), 7.98 (d, J = 8.4 Hz, 1H), 7.41 (dd, J = 1.6, 8.4Hz, 1H), 7.38 (s, 1H), 7.26 (d, J = 8.8 Hz, 1H), 7.00 (d, J = 4.0 Hz, 1H), 6.87 (dd, J = 7.2, 8.8 Hz, 1H), 6.69 (d, J = 4.0 Hz, 1H), 6.00 (d, J = 7.2 Hz, 1H), 4.21-4.08 (m, 1H), 4.00-3.90 (m, 1H), 3.63 (s, 3H), 3.51 (s, 3H). |
| 4-284 | 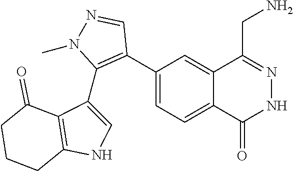 [SMILES: Cn1ncc(-c2ccc3c(=O)[nH]nc(CN)c3c2)c1-c1c[nH]c2c1C(=O)CCC2] | 4D | 4-6 | 4-(aminomethyl)-6-(1-methyl-5-(4-oxo- 4,5,6,7-tetrahydro-1H-indol-3-yl)-1H- pyrazol-4-yl)phthalazin-1(2H)-one. LCMS [M + 1]⁺ = 389.2. ¹H NMR (400 MHz, DMSO-d₆) δ = 12.78 (s, 1H), 11.84 (s, 1H), 8.44 (s, br, 3H), 8.07 (m, 2H), 7.69 (m, 2H), 6.88 (d, J = 2 Hz, 1H), 4.24-4.29 (m, 2H), 3.59 (s, 3H), 2.86-2.93 (m, 2H), 2.33 (m, 2H), 2.08 (m, 2H). |
| 4-285 | 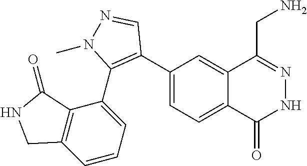 [SMILES: Cn1ncc(-c2ccc3c(=O)[nH]nc(CN)c3c2)c1-c1cccc2c1C(=O)NC2] | 4D | 4-6 | 4-(aminomethyl)-6-[1-methyl-5-(3- oxoisoindolin-4-yl)pyrazol-4-yl]-2H- phthalazin-1-one. LCMS [M + 1]⁺ = 387.2. ¹H NMR (400 MHz, DMSO-d₆) δ = 12.78 (s, 1H), 8.72 (s, 1H), 8.54 (br s, 3H), 8.20 (s, 1H), 8.01 (d, J = 8.4 Hz, 1H), 7.80 (d, J = 7.6 Hz, 1H), 7.71 (t, J = 7.6 Hz, 1H), 7,54 (s, 1H), 7.47 (dd, J = 1.2, 8.4 Hz, 1H), 7.31 (d, J = 7.2 Hz, 1H), 4.63-4.44 (m, 2H), 4.16-4.01 (m, 2H), 3.59 (s, 3H). |
| 4-286 | 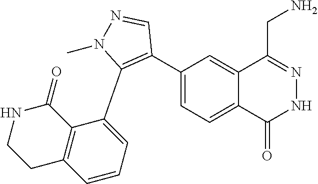 [SMILES: Cn1ncc(-c2ccc3c(=O)[nH]nc(CN)c3c2)c1-c1cccc2c1C(=O)NCC2] | 4D | 4-6 | 4-(aminomethyl)-6-(1-methyl-5-(1-oxo- 1,2,3,4-tetrahydroisoquinolin-8-yl)-1H- pyrazol-4-yl)phthalazin-1(2H)-one. LCMS [M + 1]⁺ = 401.2. ¹H NMR (400 MHz, DMSO-d₆) δ = 12.76 (s, 1H), 8.67 (s, br 3H), 8.15 (s, 1H), 7.98-8.02 (m, 2H), 7.58 (m, 2H), 7.45-7.50 (m, 2H), 7.14 (d, J = 4 Hz, 1H), 4.06 (m, 2H), 3.52 (s, 3H), 3.35 (m, 2H), 3.00-3.15 (m, 2H). |
| 4-287 | 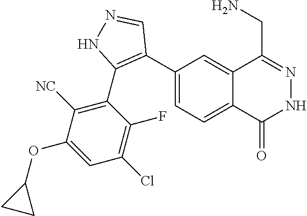 [SMILES: N#Cc1c(OC2CC2)cc(Cl)c(F)c1-c1[nH]ncc1-c1ccc2c(=O)[nH]nc(CN)c2c1] | 4D | 4-6 | 2-(4-(4-(aminomethyl)-1-oxo-1,2- dihydrophthalazin-6-yl)-1H-pyrazol-5-yl)-4- chloro-6-cyclopropoxy-3-fluorobenzonitrile LCMS [M + 1]⁺ 451.0; ¹H NMR (400 MHz, DMSO-d₆) δ = 13.88 (br s, 1H), 12.88 (s, 1H), 8.63 (br s, 1H), 8.44 (br s, 3H), 8.15 (d. J = 8.0 Hz, 1H), 7.91-7.81 (m, 2H), 7.54 (dd, J = 1.2, 8.4 Hz, 1H), 4,36 (br s, 2H), 4.22-4.12 (m, 1H), 0.96-0.85 (m, 2H), 0.81 (br s, 2H) |
| 4-288 | 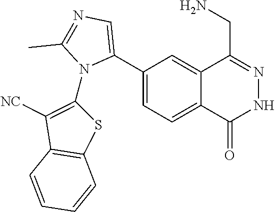 [SMILES: Cc1ncc(-c2ccc3c(=O)[nH]nc(CN)c3c2)n1-c1sc2ccccc2c1C#N] | 4D | 4-25 | 2-(5-(4-(aminomethyl)-1-oxo-1,2- dihydrophthalazin-6-yl)-2-methyl-1H- imidazol-1-yl)benzo[b]thiophene-3- carbonitrile LCMS [M + 1]⁺ 413.2; ¹H NMR (400 MHz, DMSO-d₆) δ = 12.99 (s, 1 H) 8.71 (br s, 3 H) 8.27-8.35 (m, 1 H) 8.24 (s, 1 H) 8.09- 8.18 (m, 2 H) 7.87-7.98 (m, 1 H) 7.62- 7.73 (m, 2 H) 7.54 (d, J = 9.2 Hz, 1 H) 4.32 (br d, J = 5.2 Hz, 2 H) 2.63 (s, 3 H) |
| 4-289 | 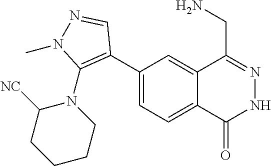 [SMILES: Cn1ncc(-c2ccc3c(=O)[nH]nc(CN)c3c2)c1N1CCCCC1C#N] | 4D | 4-25 | 1-(4-(4-(aminomethyl)-1-oxo-1,2- dihydrophthalazin-6-yl)-1-methyl-1H- pyrazol-5-yl)piperidine-2-carbonitrile LCMS [M + 1]⁺ 364.2; ¹H NMR (400 MHz, DMSO-d₆) δ = 12.91 (s, 1H), 8.46 (br s, 3H), 8.30 (d, J = 8.4 Hz, 1H), 8.08 (dd, J = 1.2, 8.4 Hz, 1H), 7.97 (d, J = 1.2 Hz, 1H), 7.80 (s, 1H), 4.58 (br s, 1H), 4.49 (br d, J = 5.6 Hz, 2H), 3.83 (s, 3H), 3.24-3.13 (m, 1H), 3.02 (br d, J = 12.0 Hz, 1H), 2.04- 1.94 (m, 1H), 1.89-1.82 (m, 1H), 1.80- 1.71 (m, 1H), 1.68-1.54 (m, 3H) |
| 4-290 | 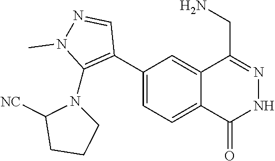 [SMILES: Cn1ncc(-c2ccc3c(=O)[nH]nc(CN)c3c2)c1N1CCCC1C#N] | 4D | 4-25 | 1-(4-(4-(aminomethyl)-1-oxo-1,2- dihydrophthalazin-6-yl)-1-methyl-1H- pyrazol-5-yl)pyrrolidine-2-carbonitrile LCMS [M + 1]⁺ 350.2; ¹H NMR (400 MHz, CD₃OD) δ = 8.42 (d, J = 8.2 Hz, 1H), 8.07 (s, 1H), 8.04 (dd, J = 1.4, 8.2 Hz, 1H), 7.91 (s, 1H), 4.63 (s, 2H), 4.51 (dd, J = 3.5, 8.0 Hz, 1H), 3.85 (s, 3H), 3.54 (td, J = 5.9, 8.2 Hz, 1H), 2.59-2.44 (m, 1H), 2.40-2.32 (m, 1H), 2.24 (br t, J = 7.2 Hz, 2H) |
| 4-291 | 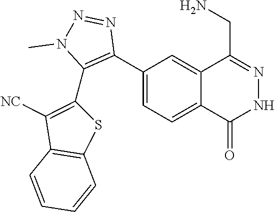 [SMILES: Cn1nnc(-c2ccc3c(=O)[nH]nc(CN)c3c2)c1-c1sc2ccccc2c1C#N] | 4B | 4-6 | 2-(4-(4-(aminomethyl)-1-oxo-1,2- dihydrophthalazin-6-yl)-1-methy1-1H-1,2,3- triazol-5-yl)benzo[b]thiophene-3- carbonitrile LCMS [M + 1]⁺ 414.1; ¹H NMR (400 MHz, MeOD-d₄) δ = 8.34-8.28 (m, 2H), 8.21- 8.15 (m, 1H), 8.06-7.99 (m, 1H), 7.82- 7.77 (m, 1H), 7.74-7.66 (m, 2H), 4.50 (s, 2H), 4.20 (s, 3H) |

### Example 4-253

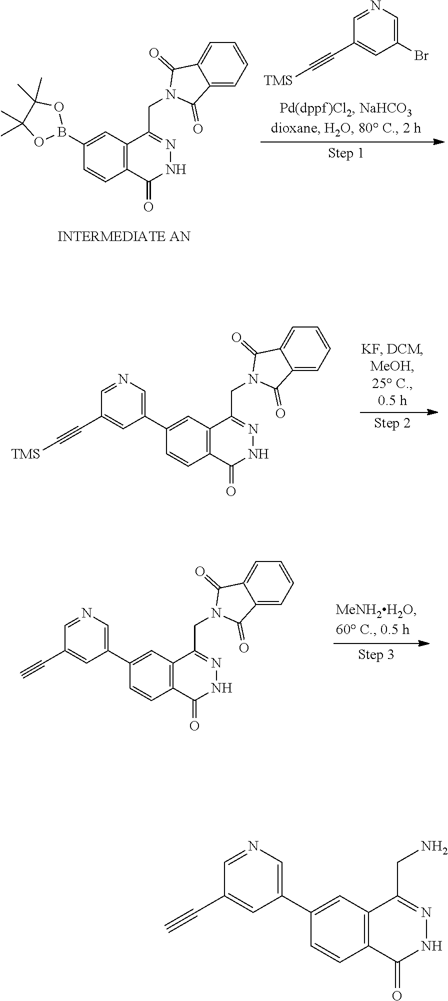 [SMILES: CC1(C)OB(c2ccc3c(=O)[nH]nc(CN4C(=O)c5ccccc5C4=O)c3c2)OC1(C)C ; C[Si](C)(C)C#Cc1cncc(Br)c1 ; C[Si](C)(C)C#Cc1cncc(-c2ccc3c(=O)[nH]nc(CN4C(=O)c5ccccc5C4=O)c3c2)c1 ; C#Cc1cncc(-c2ccc3c(=O)[nH]nc(CN4C(=O)c5ccccc5C4=O)c3c2)c1 ; C#Cc1cncc(-c2ccc3c(=O)[nH]nc(CN)c3c2)c1]

Step 1: To a solution of Intermediate AN (136 mg, 315 μmol, 1.00 eq.) in dioxane (4 mL) and water (0.80 mL) was added Pd(dppf)Cl₂ (23 mg, 32 μmol, 0.10 eq.) and sodium bicarbonate (79 mg, 944 μmol, 37 μL, 3.00 eq.) and the reaction mixture was stirred at 80° C. for 2 hours. After such time the reaction was filtered, the filtrate was concentrated in vacuum to a residue, the residue was purified by prep-TLC (petroleum ether:ethyl acetate 10-100%) to give 2-[[4-oxo-7-[5-(2-trimethylsilylethynyl)-3-pyridyl]-3H-phthalazin-1-yl]methyl] isoindoline-1,3-dione (100 mg, 209 μmol, 66% yield) as a brown solid. ¹H NMR (400 MHz, DMSO-d₆) δ=12.32 (s, 1H), 8.96 (d, J=2.4 Hz, 1H), 8.55 (d, J=2.0 Hz, 1H), 8.36-8.31 (m, 2H), 8.14 (s, 2H), 7.79-7.74 (m, 2H), 7.73-7.69 (m, 2H), 5.19 (s, 2H), 0.09 (s, 9H).

Step 2: To a solution of 2-[[4-oxo-7-[5-(2-trimethylsilylethynyl)-3-pyridyl]-3H-phthalazin-1-yl]methyl]isoindoline-1,3-dione (100 mg, 209 μmol, 1.00 eq.) in methanol (2 mL) and dichloromethane (2 mL) was added potassium fluoride (36 mg, 627 μmol, 14.7 μL, 3.00 eq.), and the reaction was stirred at 25° C. for 0.5 hour. The reaction was concentrated under vacuum and the residue was triturated with water (5 mL), filtered and the filter cake was dried in vacuum to give 2-[[7-(5-ethynyl-3-pyridyl)-4-oxo-3H-phthalazin-1-yl] methyl]isoindoline-1,3-dione (80 mg, 197 μmol, 94% yield) as a brown solid. LCMS [M+1]⁺=407.0.

Step 3: A mixture of 2-[[7-(5-ethynyl-3-pyridyl)-4-oxo-3H-phthalazin-1-yl]methyl]isoindoline-1,3-dione (80 mg, 197 μmol, 1.00 eq.) in methylamine aqueous solution (4 mL) was stirred at 60° C. for 0.5 hour. After such time the reaction was concentrated under vacuum and the residue was purified by prep-HPLC (column: Phenomenex Synergi C18 150×25×10 μm; mobile phase: [water (0.05% HCl)-ACN]; B %: 0%-25%, 10 min) and lyophilization to give 4-(aminomethyl)-6-(5-ethynyl-3-pyridyl)-2H-phthalazin-1-one, example 4-253 (10 mg, 32.1 μmol, 16% yield) as a yellow solid. LCMS [M+1]⁺=277.2. ¹H NMR (400 MHz, DMSO-d₆) δ=13.01 (s, 1H), 9.17 (d, J=2.4 Hz, 1H), 8.81 (d, J=1.6 Hz, 1H), 8.60-8.49 (m, 4H), 8.41-8.37 (m, 1H), 8.36-8.30 (m, 2H), 4.65-4.55 (m, 3H).

### Example 4-254

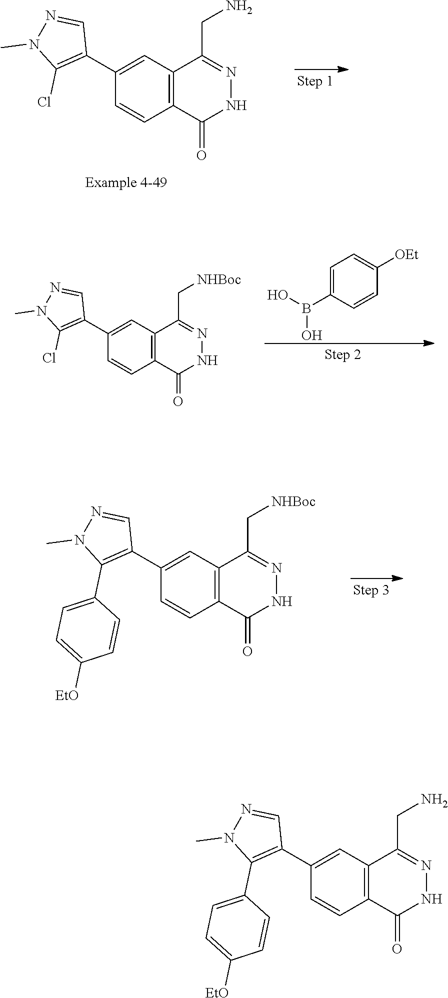 [SMILES: Cn1ncc(-c2ccc3c(=O)[nH]nc(CN)c3c2)c1Cl ; Cn1ncc(-c2ccc3c(=O)[nH]nc(CNC(=O)OC(C)(C)C)c3c2)c1Cl ; CCOc1ccc(B(O)O)cc1 ; CCOc1ccc(-c2c(-c3ccc4c(=O)[nH]nc(CNC(=O)OC(C)(C)C)c4c3)cnn2C)cc1 ; CCOc1ccc(-c2c(-c3ccc4c(=O)[nH]nc(CN)c4c3)cnn2C)cc1]

Step 1: A mixture of 4-(aminomethyl)-6-(5-chloro-1-methyl-pyrazol-4-yl)-2H-phthalazin-1-one, Example 4-49 (45 mg, 155 μmol, 1.00 eq.), di-tert-butyl dicarbonate (68 mg, 311 μmol, 71 μL, 2.00 eq.) and triethylamine (47 mg, 466 μmol, 65 μL, 3.00 eq.) in dichloromethane (1.0 mL) was purged with nitrogen 3 times and stirred at 25° C. for 3 hours. After such time the mixture was concentrated under reduced pressure to a residue. The mixture was triturated with petroleum ether (3 mL) and filtered to give tert-butyl N-[[7-(5-chloro-1-methyl-pyrazol-4-yl)-4-oxo-3H-phthalazin-1-yl]methyl]carbamate (40 mg, 96 μmol, 62% yield) as a yellow solid. LCMS [M+1]⁺=390.2. ¹H NMR (400 MHz, MeOD) δ=8.41 (d, J=8.4 Hz, 1H), 8.33 (s, 1H), 8.19 (br d, J=8.4 Hz, 1H), 8.05 (s, 1H), 4.62 (s, 2H), 3.96-3.92 (s, 3H), 1.48-1.40 (s, 9H).

Step 2: A mixture of (4-ethoxyphenyl)boronic acid (16 mg, 98.5 μmol, 1.20 eq.), tert-butyl N-[[7-(5-chloro-1-methyl-pyrazol-4-yl)-4-oxo-3H-phthalazin-1-yl]methyl]carbamate (32 mg, 82 μmol, 1.00 eq.), sodium carbonate (26 mg, 246 μmol, 3.00 eq.) and Pd(dppf)Cl₂ (6 mg, 8 μmol, 0.10 eq.) in dioxane (3 mL) and water (0.6 mL) was purged with nitrogen 3 times and stirred at 110° C. for 12 hours. The mixture was concentrated under reduced pressure and the residue was purified by column chromatography (SiO₂, petroleum ether/ethyl acetate 10-100%) to give tert-butyl-N-[[7-[5-(4-ethoxyphenyl)-1-methyl-pyrazol-4-yl]-4-oxo-3H-phthalazin-1-yl]methyl]carbamate (28 mg, 45 μmol, 54% yield) as a white solid. LCMS [M+1]⁺=476.2.

Step 3: A mixture of tert-butyl-N-[[7-[5-(4-ethoxyphenyl)-1-methyl-pyrazol-4-yl]-4-oxo-3H-phthalazin-1-yl]methyl]carbamate (28 mg, 59 μmol, 1.00 eq.) and trifluoroacetic acid (1.08 g, 9.45 mmol, 700 μL, 161 eq.), in dichloromethane (3 mL) was purged with nitrogen 3 times and stirred at 25° C. for 1 hour. After such time the mixture was concentrated under reduced pressure and the residue was purified by prep-HPLC (Phenomenex Gemini-NX C18 75×30 mm×3 μm; mobile phase: [water (0.1% TFA)-ACN]; B %: 20%-30/o, 7 min) to give 4-(aminomethyl)-6-[5-(4-ethoxyphenyl)-1-methyl-pyrazol-4-yl]-2H-phthalazin-1-one, Example 4-254 (25 mg, 49 μmol, 83% yield, TFA salt) as an off-white solid. LCMS [M+1]⁺=376.1. ¹H NMR (400 MHz, DMSO-d₆) δ=12.84 (s, 1H), 8.34 (br s, 3H), 8.12 (s, 1H), 8.07 (d, J=8.4 Hz, 1H), 7.79 (d, J=1.2 Hz, 1H), 7.51 (dd, J=1.6, 8.4 Hz, 1H), 7.33 (d, J=8.8 Hz, 2H), 7.08 (d, J=8.8 Hz, 2H), 4.31 (br d, J=4.4 Hz, 2H), 4.11 (q, J=6.8 Hz, 2H), 3.72 (s, 3H), 1.37 (t, J=6.8 Hz, 3H).

### Examples 5-1 and 5-2

### General Coupling Method for the Preparation of Examples 5-1 and 5-2

### Coupling Method 5:

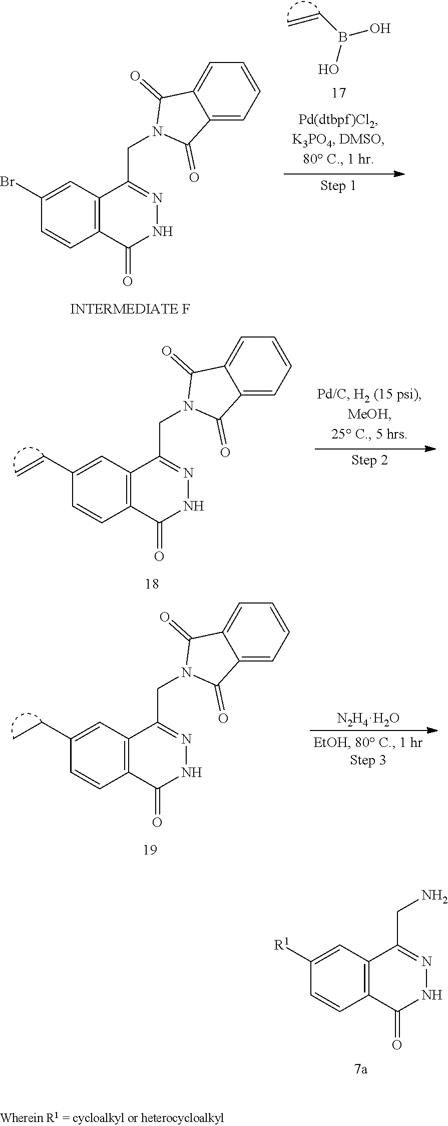

Step 1: A mixture of Intermediate F (200 mg, 521 μmol, 1.00 eq.), R^{1a}-boronic acid 17 where R^{1a} is cycloalkenyl or heterocycloalkenyl (781 μmol, 1.50 eq.), di-tert-butyl(cyclopentyl)phosphane;dichloropalladium;iron (34 mg, 52 μmol, 0.10 eq.), potassium phosphate (221 mg, 1.04 mmol, 2.00 eq.) in dimethylsulfoxide (2.5 mL) was purged with nitrogen 3 times and the mixture stirred at 80° C. for 1 hour and cooled to room temperature. Water (50 mL) was then added and the mixture extracted with ethyl acetate (40 mL×3). The combined organic extracts were washed with brine (50 mL), dried over anhydrous sodium sulfate, filtered, and concentrated in vacuo and the residue purified by column chromatography (SiO₂, dichloromethane/methanol 0 to 10% methanol) to give the corresponding coupled product 18 as a light-yellow solid.

Step 2: To a solution of the corresponding coupled product 18 (296 μmol, 1.00 eq.) in methanol (6 mL) was added 10% palladium on activated carbon (10 mg). The mixture was purged with hydrogen several times and stirred under a hydrogen atmosphere (15 psi) at 25° C. for 5 hours. After such time the reaction mixture was filtered and concentrated to give the corresponding reduced product 19 as a white solid which was used in the next step without further purification.

Step 3: A mixture of the corresponding reduced product 19 and hydrazine hydrate (27 mg, 536 μmol, 26 μL) in ethanol (5 mL) was stirred at 80° C. for 1 hour. The reaction mixture was evaporated and the residue was purified by prep-HPLC (Phenomenex Synergi C18 150×25×10 μm; mobile phase: [water (0.05% HCl)-ACN]; B %: 16%-36%, 9 min) to give the desired R¹ amino compounds where R¹ is cycloalkyl or heterocycloalkyl.

Following the teachings of the General Reaction Schemes, the coupling method 5 and the Intermediates disclosed herein, the Examples 5-1 to 5-2 are prepared as shown in Table 5:

TABLE 5

| Example | Structure | Compound Name and Characterization |
|---|---|---|
| 5-1 | 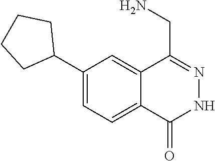 [SMILES: NCc1n[nH]c(=O)c2ccc(C3CCCC3)cc12] | 4-(aminomethyl)-6-cyclopentylphthalazin-1(2H)-one; LCMS [M + 1]⁺ 244.2; ¹H NMR (400 Hz, DMSO-d₆) δ = 12.83 (s, 1H), 8.43 (br s, 3H), 8.22 (d, J = 8.4 Hz, 1H), 7.83 (d, J = 8.4 Hz, 1H), 7.78 (s, 1H), 4.47 (br d, J = 4.8 Hz, 2H), 3.27-3.16 (m, 1H), 2.18-2.05 (m, 2H), 1.84 (m, 2H), 1.77-1.61 (m, 4H). |
| 5-2 | 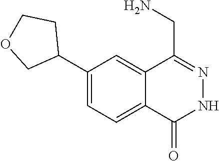 [SMILES: NCc1n[nH]c(=O)c2ccc(C3CCOC3)cc12] | (R,S)-4-(aminomethyl)-6-(THF-3-yl)phthalazin-1(2H)- one; LCMS [M + 1]⁺ 246.1; ¹H NMR (400 Hz, DMSO- d₆) δ = 12.86 (s, 1H), 8.53 (s, 3H), 8.24 (d, J = 8.8 Hz, 1H), 7.89-7.81 (m, 2H), 4.46 (br d, J = 5.2 Hz, 2H), 4.14-3.98 (m, 2H), 3.85 (q, J = 7.6 Hz, 1H), 3.75-3.58 (m, 2H), 3.44-3.36 (m, 1H), 2.11-2.02 (m, 1H). |

### Preparation of Examples 6-1 to 6-5

### Example 6-1

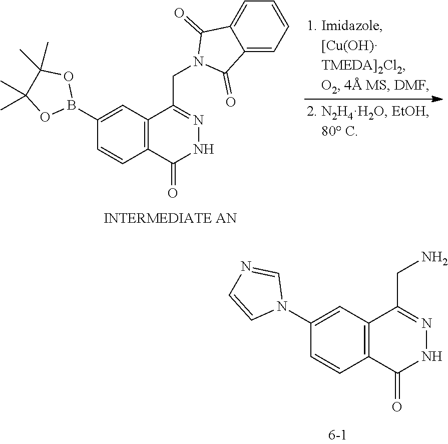 [SMILES: CC1(C)OB(c2ccc3c(=O)[nH]nc(CN4C(=O)c5ccccc5C4=O)c3c2)OC1(C)C ; NCc1n[nH]c(=O)c2ccc(-n3ccnc3)cc12]

Step 1: A mixture of Intermediate AN (86 mg, 200 μmol, 1.00 eq.), imidazole (68 mg, 1.00 mmol, 5.00 eq.), chloro(hydroxy)copper-N,N,N′,N′-tetramethylethane-1,2-diamine (9 mg, 20 μmol, 0.10 eq.) and 4 Å molecular sieve (10 mg) in DMF (5 mL) was degassed and purged with oxygen 3 times. The mixture was then stirred at 25° C. for 12 hours under an oxygen atmosphere (15 psi). After such time the mixture was filtered and concentrated under reduced pressure and the residue purified by prep-HPLC (Phenomenex Synergi C18 150×25×10 μm; mobile phase: [water (0.225% FA)-ACN]; B %: 3%-33%, 10 min) to give 2-[(7-imidazol-1-yl-4-oxo-3H-phthalazin-1-yl)methyl]isoindoline-1,3-dione (4 mg, 10.8 μmol, 5% yield) as a white solid LCMS [M+1]⁺=372.1.

Step 2: To a solution of 2-[(7-imidazol-1-yl-4-oxo-3H-phthalazin-1-yl)methyl]isoindoline-1,3-dione (3 mg, 8 μmol, 1.00 eq.) in ethyl alcohol (1.0 mL) was added hydrazine hydrate (4 mg, 81 μmol, 3 μL, 10.0 eq.). The mixture was stirred at 80° C. for 1 hour and then the mixture was concentrated under reduced pressure. The concentrated residue was then purified by prep-HPLC (Phenomenex Synergi C18 150×25 10 μm; mobile phase: [water (0.05% HCl)-ACN]; B %: 0/6-20%, 9 min) to give 4-(aminomethyl)-6-imidazol-1-yl-2H-phthalazin-1-one, example 6-1 (1.4 mg, 5 μmol, 61% yield) as an off-white solid. LCMS [M+1]⁺ =242.1. ¹H NMR (400 MHz, DMSO-d₆) δ=13.12 (s, 1H), 9.71-9.57 (s, 1H), 8.70-8.61 (brs, 31H), 8.49 (d, J=8.8 Hz 1H), 8.47 (s, 1H), 8.44 (s, 1H), 8.33 (dd, J=2.0, 8.8 Hz, 1H), 7.77 (br s, 1H), 4.53 (br d, J=5.6 Hz, 2H).

### Example 6-2

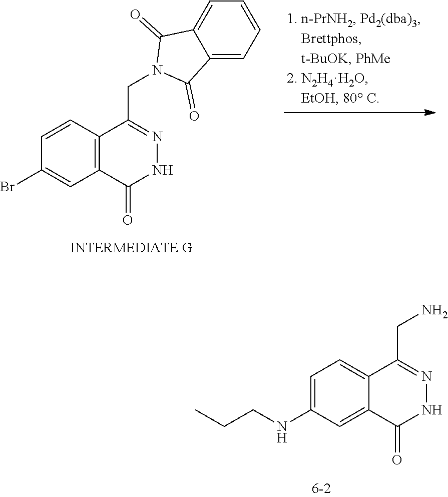 [SMILES: O=C1c2ccccc2C(=O)N1Cc1n[nH]c(=O)c2cc(Br)ccc12 ; CCCNc1ccc2c(CN)n[nH]c(=O)c2c1]

Step 1: To a solution of Intermediate G (50 mg, 130 μmol, 1.00 eq.) and propan-1-amine (9 mg, 156 μmol, 13 μL, 1.20 eq.) in toluene (2 mL) was added Pd₂(dba)₃ (24 mg, 26 μmol, 0.20 eq.), potassium tert-butoxide (29 mg, 260 μmol, 2.00 eq.) and BrettPhos (140 mg, 260 μmol, 2.00 eq.) under a nitrogen atmosphere. The mixture was stirred at 100° C. for 12 hours. After such time the mixture was concentrated in vacuo and the residue dissolved in a water:methyl alcohol 2:1 mixture (3 mL) and filtered. The filtrate was concentrated in vacuo to give 2-[[4-oxo-6-(propylamino)-3H-phthalazin-1-yl]methylcarbamoyl]benzoic acid (35 mg, crude) as a yellow solid, which was used in the next step without further purification. LCMS [M+1]=381.1.

### Example 6-3

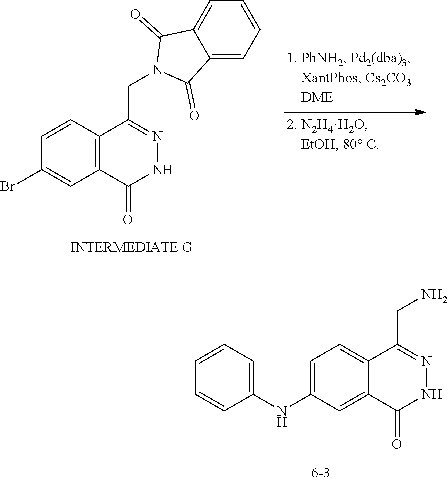 [SMILES: O=C1c2ccccc2C(=O)N1Cc1n[nH]c(=O)c2cc(Br)ccc12 ; NCc1n[nH]c(=O)c2cc(Nc3ccccc3)ccc12]

Step 1 To a solution of Intermediate G (200 mg, 521 μmol, 1.00 eq.) and aniline (58 mg, 624 μmol, 57.0 μL, 1.20 eq.) in DME (3 mL) was added cesium carbonate (678 mg, 2.08 mmol, 4.00 eq.), Pd₂(dba)₁ (48 mg, 52 μmol, 0.10 eq.) and Xantphos (331 mg, 572 μmol, 1.10 eq.). The mixture was stirred at 95° C. for 12 hours under nitrogen atmosphere. After such time the mixture was concentrated in vacuo and the residue taken up in water (5 mL) and methyl alcohol (5 mL) then filtered. The filtrate was concentrated in vacuum to give 2-[(6-anilino-4-oxo-3H-phthalazin-1-yl)methylcarbamoyl]benzoic acid (200 mg, crude) as a yellow solid, which was used in the next step without further purification.

Step 2: To a solution of 2-[(6-anilino-4-oxo-3H-phthalazin-1-yl)methylcarbamoyl]benzoic acid (50 mg, crude) in ethyl alcohol (1 mL) was added hydrazine hydrate (12.9 mg, 252 μmol, 12.5 μL). The mixture was stirred at 80° C. for 12 hours. After such time the mixture was concentrated in vacuo and the residue purified by prep-HPLC (Phenomenex Synergi C18 150×25×10 μm; mobile phase: [water (0.05% HCl)-ACN]; B %: 2%-32%, 10 min) to give 4-(aminomethyl)-7-anilino-2H-phthalazin-1-one, example 6-3 (10 mg, 33 μmol, 24% yield) as a yellow solid. LCMS [M+1]⁺=267.0. ¹H NMR (400 MHz, DMSO-d₆) δ=12.63 (s, 1H), 9.17 (s, 1H), 8.43 (br s, 3H), 7.82 (d, J=8.8 Hz, 1H), 7.77 (d, J=2.4 Hz, 1H), 7.52 (dd, J=2.4, 8.8 Hz, 1H), 7.42-7.36 (m, 2H), 7.28-7.23 (m, 2H), 7.07 (t, J=7.2 Hz, 1H), 4.37 (br s, 2H).

### Example 6-4

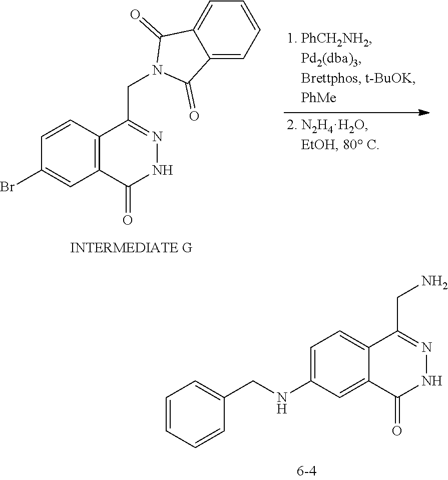 [SMILES: O=C1c2ccccc2C(=O)N1Cc1n[nH]c(=O)c2cc(Br)ccc12 ; NCc1n[nH]c(=O)c2cc(NCc3ccccc3)ccc12]

Step 1: To a solution of Intermediate G (50 mg, 130 μmol, 1.00 eq.) and phenylmethanamine (17 mg, 156 μmol, 17.0 μL, 1.20 eq.) in toluene (2 mL) was added Pd₂(dba)₃ (24 mg, 26 μmol, 0.20 eq.), potassium tert-butoxide (29 mg, 260 μmol, 2.00 eq.) and BrettPhos (140 mg, 260 μmol, 2.00 eq.) under a nitrogen atmosphere. The mixture was stirred at 100° C. for 12 hours. After such time the mixture was concentrated in vacuo and the residue dissolved in a water:methyl alcohol 2:1 mixture (3 mL) and filtered. The filtrate was concentrated in vacuo to give 2-[[6-(benzylamino)-4-oxo-3H-phthalazin-1-yl]methylcarbamoyl]benzoic acid (35 mg, crude) as a yellow solid, which was used directly in the next step without further purification. LCMS [M+1]⁺=429.1. To a solution of 2-[[6-(benzylamino)-4-oxo-3H-phthalazin-1-yl]methylcarbamoyl] benzoic acid (35 mg, crude) in ethyl alcohol (1 mL) was added hydrazine hydrate (9 mg, 171 μmol, 8 μL). The mixture was stirred at 80° C. for 12 hours. After such time the mixture was concentrated in vacuo and the residue purified by prep-HPLC (Phenomenex Synergi C18 150×25×10 μm; mobile phase: [water (0.05% HCl)-ACN]; B %: 2%-32%, 10 min) to give 4-(aminomethyl)-7-(benzylamino)-2H-phthalazin-1-one, example 6-4 (7 mg, 22 μmol, 17% yield) as a yellow solid. LCMS [M+1]⁺=281.2. ¹H NMR (400 MHz, DMSO-d₆) δ=12.49 (s, 1H), 8.32 (br s, 3H), 7.67 (d, J=8.8 Hz, 1H), 7.58-7.50 (m, 1H), 7.37-7.32 (m, 4H), 7.27-7.19 (m, 3H), 4.45 (br s, 2H), 4.31 (br d, J=6.0 Hz, 2H).

### Example 6-5

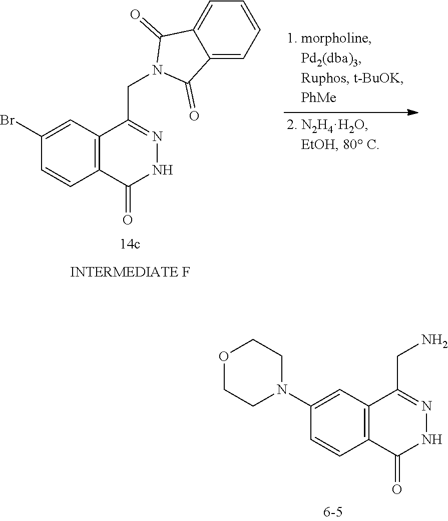 [SMILES: O=C1c2ccccc2C(=O)N1Cc1n[nH]c(=O)c2ccc(Br)cc12 ; NCc1n[nH]c(=O)c2ccc(N3CCOCC3)cc12]

Step 1: To a solution of Intermediate F (50 mg, 130 μmol, 1.00 eq.) and morpholine (17 mg, 195 μmol, 17 μL, 1.50 eq.) in toluene (2 mL) was added Pd₂(dba)₃ (12 mg, 13 μmol, 0.10 eq.), potassium tert-butoxide (44 mg, 390 μmol, 3.00 eq.) and RuPhos (121 mg, 260 μmol, 2.00 eq.) under a nitrogen atmosphere. The mixture was stirred at 100° C. for 2 hours. After such time the mixture was concentrated in vacuo and the residue dissolved in a water:methyl alcohol 2:1 mixture (3 mL) and filtered. The filtrate was concentrated in vacuo to give 2-(morpholine-4-carbonyl)-N-[(7-morpholino-4-oxo-3H-phthalazin-1-yl)methyl]benzamide (35.0 mg, crude) as a yellow solid which was used directly in the next step without further purification. LCMS [M+1]⁺=478.2. To a solution of 2-(morpholine-4-carbonyl)-N-[(7-morpholino-4-oxo-3H-phthalazin-1-yl)methyl]benzamide (35 mg, crude) in ethyl alcohol (1 mL) was added hydrazine hydrate (9 mg, 176 μmol, 9 μL). The mixture was stirred at 80° C. for 12 hours. After such time the mixture was concentrated in vacuo and the residue purified by prep-HPLC (Phenomenex Synergi C18 150-25×10 μm; mobile phase: [water (0.05% HCl)-ACN]; B %: 3%-23%, 9 min) to give 4-(aminomethyl)-6-morpholino-2H-phthalazin-1-one, example 6-5 (9 mg, 28 μmol, 32% yield) as a white solid. LCMS [M+1]⁺=261.0. ¹H NMR (400 MHz, DMSO-d₄) δ=12.56 (s, 1H), 8.50 (br s, 3H), 8.08 (d, J=9.2 Hz, 1H), 7.51 (dd, J=2.4, 9.2 Hz, 1H), 7.09 (d, J=2.4 Hz, 1H), 4.38 (q, J=5.6 Hz, 2H), 3.79-3.76 (m, 4H), 3.47-3.41 (m, 4H).

### General Coupling Method and Purification Methods for the Preparation of Examples 7-1 to 7-6

### General Method 7

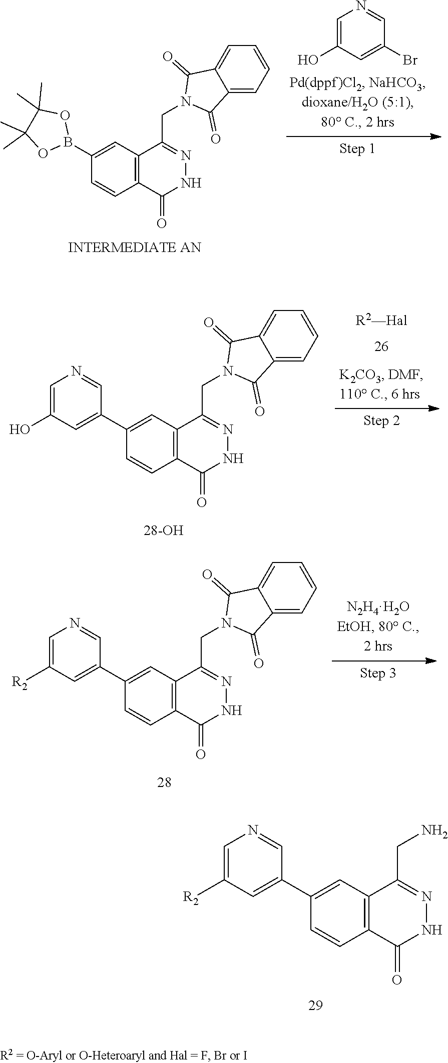

Step 1: A mixture of Intermediate AN (150 mg, 348 μmol, 1.00 eq.), 5-bromopyridin-3-ol (522 μmol, 1.5 eq), Pd(dppf)Cl₂ (26 mg, 35 μmol, 0.10 eq.), sodium bicarbonate (58 mg, 696 μmol, 27 μL, 2.00 eq.) in dioxane (1 mL) and water (0.2 mL) was degassed and purged with nitrogen 3 times. The mixture was stirred at 80° C. for 3 hours under a nitrogen atmosphere. Upon completion the reaction mixture was diluted with water (30 mL) and extracted with ethyl acetate (30 mL×3). The aqueous phase was filtered, and the filter cake dried to give the corresponding R²-Pyridyl-Suzuki coupling product 28-OH which was used directly in the next step without further purification.

Step 2: To a solution of Pyridyl-Suzuki coupling product 28-OH (80 mg, crude) in DMF (1 mL) was added potassium carbonate (83 mg, 602 μmol) and aryl/heteroaryl-substituted fluoride/bromide 26 (54 mg, 402 μmol). The mixture was stirred at 120° C. for 6 hours to furnish R²-Pyridyl-S_{N}Ar coupling product 28. The reaction mixture was used in the next step directly without further purification.

Step 3: To a solution of corresponding R²-Pyridyl-S_{N}Ar coupling product 28 (50 mg, crude) in ethyl alcohol (10 mL) was added hydrazine hydrate (11.0 mg, 210 μmol, 10 μL). The mixture was stirred at 80° C. for 2 hours and the cooled mixture was concentrated in vacuo. The concentrated residue 29 was purified by prep-HPLC according to one of the purification methods 7-1 through 7-3 to return desired compounds shown in table 7.

### Purification Methods (PM)

PM 7-1: column: Phenomenex Synergi C18 150×25 mm×10 μm; mobile phase: [water (0.05% HCN)-ACN]; B %: 5%-25%, 11 min.
PM 7-2: column: Waters Xbridge 150×25 mm×5 μm; mobile phase: [water (100 mM NH₄HCO₃)ACN]; B %: 2%-32%, 10 min,
PM 7-3: column: Shim-pack C18 150.25 mm×10 μm; mobile phase: [water (0.1% TFA)-ACN]; B %: 5) 3,208, 10 min.

Following the teachings of the General Reaction Schemes, coupling method 7 and using purification methods 7-1 through 7-3 and the Intermediates disclosed herein, the Examples 7-1 to 7-6 are prepared as shown in Table 7.

TABLE 7

| Examples | Structure | PM | Compound Name and Characterization |
|---|---|---|---|
| 7-1 | 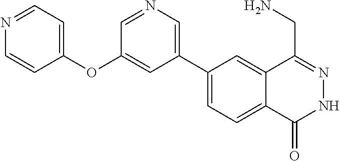 [SMILES: NCc1n[nH]c(=O)c2ccc(-c3cncc(Oc4ccncc4)c3)cc12] | 7-3 | 4-(aminomethyl)-6-(5-(pyridin-4-yloxy)pyridin- 3-yl)phthalazin-1(2H)-one LCMS [M + 1]⁺ = 346.0; ¹H NMR (400 MHz, DMSO-d₆) δ = 13.02 (s, 1H), 9.17 (s, 1H), 8.69 (s, 1H), 8.63 (br d, J = 5.2 Hz, 2H), 8.46-8.37 (m, 5H), 8.36 (s, 1H), 8.33 (s, 1H), 7.25 (br s, 2H), 4.59 (br d, J = 5.6 Hz, 2H). |
| 7-2 | 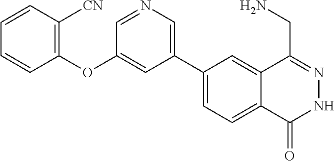 [SMILES: N#Cc1ccccc1Oc1cncc(-c2ccc3c(=O)[nH]nc(CN)c3c2)c1] | 7-2 | 2-((5-(4-(aminomethyl)-1-oxo-1,2-dihydro- phthalazin-6-yl)pyridin-3-yl)oxy)benzonitrile LCMS [M + 1]⁺ = 370.0; ¹H NMR (400 MHz, DMSO-d₆) δ = 8.99 (s, 1H), 8.55 (d, J = 2.8 Hz, 1H), 8.41-8.36 (m, 2H), 8.20 (d, J = 8.4 Hz, 1H), 8.15 (s, 1H), 7.92 (d, J = 7.6 Hz, 1H), 7.73 (t, J = 8.0 Hz, 1H), 7.37 (t, J = 7.6 Hz, 1H), 7.20 (d, J = 8.4 Hz, 1H), 4.13 (s, 2H). |
| 7-3 | 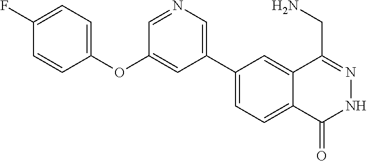 [SMILES: NCc1n[nH]c(=O)c2ccc(-c3cncc(Oc4ccc(F)cc4)c3)cc12] | 7-1 | 4-(aminomethyl)-6-(5-(4-fluorophenoxy)- pyridin-3-yl)phthalazin-1(2H)-one LCMS [M + 1]⁺ = 363.1; ¹H NMR (400 MHz, DMSO- d₆) δ = 13.00 (s, 1H), 9.02 (d, J = 1.6 Hz, 1H), 8.66-8.51 (brs, 3H), 8.47 (d, J = 2.8 Hz, 1H), 8.40-8.25 (m, 3H), 8.17 (s, 1H), 7.33-7.21 (m, 4H), 4.57 (br d, J = 5.6 Hz, 2H). |
| 7-4 | 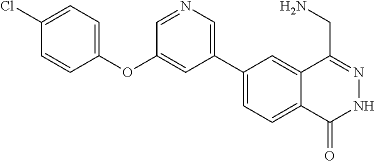 [SMILES: NCc1n[nH]c(=O)c2ccc(-c3cncc(Oc4ccc(Cl)cc4)c3)cc12] | 7-1 | 4-(aminomethyl)-6-(5-(4-chlorophenoxy)- pyridin-3-yl)phthalazin-1(2H)-one LCMS [M + 1]⁺ = 379.0; ¹H NMR (400 MHz, DMSO- d₆) δ = 13.00 (s, 1H), 9.05 (d, J = 2.0 Hz, 1H), 8.60 (br s, 3H), 8.53 (d, J = 2.4 Hz, 1H), 8.40- 8.35 (d, J = 8.0 Hz, 1H), 8.34-8.27 (m, 2H), 8.22 (t, J = 2.4 Hz, 1H), 7.54-7.45 (d, J = 8.8 Hz, 2H), 7.22-7.15 (d, J = 8.8 Hz, 2H), 4.63- 4.53 (d, J = 5.6 Hz, 2H). |
| 7-5 | 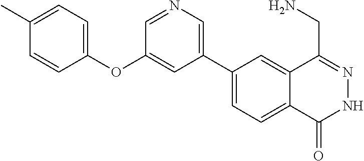 [SMILES: Cc1ccc(Oc2cncc(-c3ccc4c(=O)[nH]nc(CN)c4c3)c2)cc1] | 7-2 | 4-(aminomethyl)-6-(5-(p-tolyloxy)pyridin-3- yl)phthalazin-1(2H)-one LCMS [M + 1]⁺ = 359.3; ¹H NMR (400 MHz, DMSO-d₆) δ = 8.82 (d, J = 1.6 Hz, 1H), 8.38 (d, J = 2.4 Hz, 1H), 8.37-8.32 (m, 2H), 8.14-8.09 (m, 1H), 7.87 (s, 1H), 7.25 (d, J = 8.0 Hz, 2H), 7.05 (d, J = 8.0 Hz, 2H), 4.11 (s, 2H), 2.33 (s, 3H). |
| 7-6 | 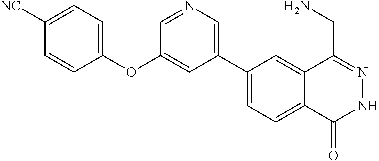 [SMILES: N#Cc1ccc(Oc2cncc(-c3ccc4c(=O)[nH]nc(CN)c4c3)c2)cc1] | 7-2 | 4-((5-(4-(aminomethyl)-1-oxo-1,2-dihydro- phthalazin-6-yl)pyridin-3-yl)oxy)benzonitrile LCMS [M + 1]⁺ = 370.0; ¹H NMR (400 MHz, DMSO-d₆) δ = 8.98 (d, J = 2.0 Hz, 1H), 8.53 (d, J = 2.8 Hz, 1H), 8.39-8.34 (m, 2H), 8.18 (dd, J = 2.0, 8.4 Hz, 1H), 8.11 (t, J = 2.4 Hz, 1H), 7.90-7.83 (dd, J = 2.0, 7.2 Hz, 2H), 7.31- 7.25 (dd, J = 2.4, 6.8 Hz, 2H), 4.12 (s, 2H). |

### Coupling Methods (CM) and Purification Methods for the Preparation of Examples 8-1 to 8-9

### CM 8A

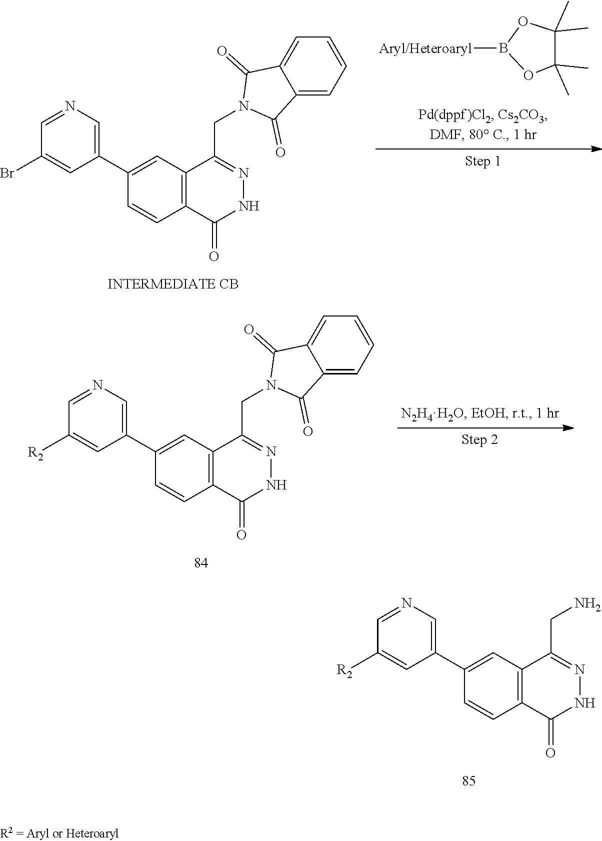

Step 1: A mixture of Intermediate CB (100 mg, 217 μmol, 1.00 eq.), an appropriate aryl/heteroaryl boronic ester (260 μmol, 1.20 eq.), cesium carbonate (141 mg, 434 μmol, 2.00 eq.) and Pd(dppf)Cl₂ (16.0 mg, 22 μmol, 0.10 eq.) in dimethylformamide (2 mL) was purged with nitrogen 3 times and stirred at 80° C. for 1 hour. Upon completion, the reaction mixture was poured into water (50 mL), filtered and filter cake dried under reduced pressure to give the appropriate R²-pyridyl coupled product 84 (113 mg, crude) as a yellow solid that was used in the next step without further purification.

Step 2: To a solution of 84 (63 mg, crude) in ethyl alcohol (10 mL) was added hydrazine hydrate (13 mg, 251 μmol, 12 μL). The mixture was stirred at 35° C. for 1 hour and upon completion the mixture was adjusted pH to 1 with hydrochloric acid (1 M, 1 mL) and concentrated under reduced pressure. The residue was diluted with hydrochloric acid (1 M, 40.0 mL) and washed with ethyl acetate (30 mL×3) and the aqueous phase was concentrated under reduced pressure. The residue was purified by prep-HPLC according to one of the purification methods 8-1 or 8-2 to give desired compounds 85.

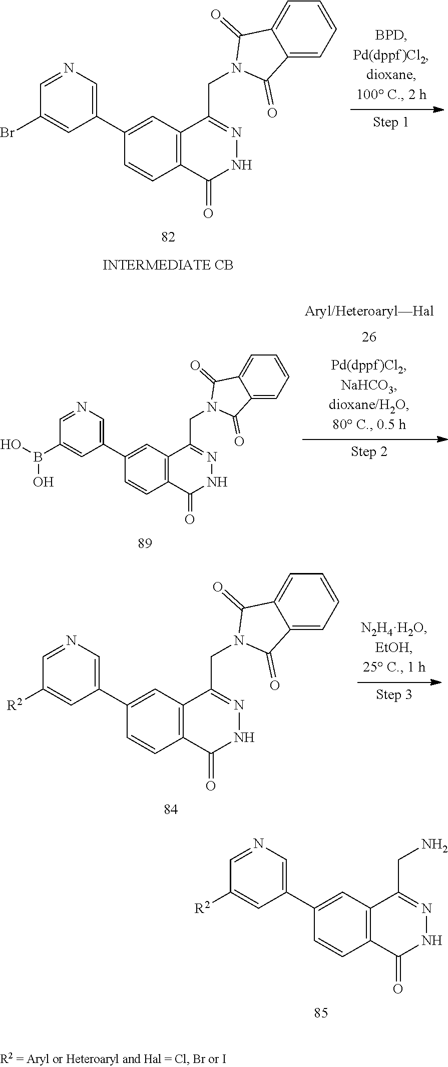

Step 1: A mixture of Intermediate CB (300 mg, 650 μmol, 1.00 eq), 4,4,5,5-tetramethyl-2-(4,4,5,5-tetramethyl-1,3,2-dioxaborolan-2-yl)-1,3,2-dioxaborolane (BPD) (198 mg, 780 μmol, 1.20 eq), Pd(dppf)Cl₂ (47 mg, 65 μmol, 0.10 eq) and potassium acetate (128 mg, 1.30 mmol, 2.00 eq) in dioxane (5 mL) was degassed and purged with nitrogen 3 times, and stirred at 100° C. for 2 hours under a nitrogen atmosphere to furnish [5-[4-[(1,3-dioxoisoindolin-2-yl)methyl]-1-oxo-2H-phthalazin-6-yl]-3-pyridyl]boronic acid 89. The reaction mixture was used directly in next step. LCMS [M+1]⁺=427.2.

Step 2: A mixture of [5-[4-[(1,3-dioxoisoindolin-2-yl)methyl]-1-oxo-2H-phthalazin-6-yl]-3-pyridyl]boronic acid 89 (130 mg, 305 μmol, 1.00 eq), an appropriate R² bromide 42 (610 μmol, 2.00 eq), Pd(dppf)Cl₂ (22 mg, 31 μmol, 0.10 eq), sodium bicarbonate (51 mg, 610 μmol, 24 μL, 2.00 eq) in dioxane (4 mL) and water (0.8 mL) was degassed and purged with nitrogen 3 times, stirred at 80° C. for 0.5 hour under a nitrogen atmosphere. Upon completion the reaction mixture was concentrated under reduced pressure to give a residue that was purified by column chromatography (SiO₂, petroleum ether/ethyl acetate 10:1 to 0:1, then dichloromethane/methanol 10/1) to give R²-pyridyl coupled product 84 (150 mg, crude) as a black solid.

Step 3: To a solution of R²-pyridyl coupled product 84 (109 mg, 215 μmol, 1.00 eq) in ethyl alcohol (3.0 mL) was added hydrazine hydrate (110 mg, 2.15 mmol, 106 μL, 10.0 eq). The reaction mixture was stirred at 25° C. for 1 hour. Upon completion the reaction mixture was concentrated under reduced pressure and the residue was acidified to pH 3 with hydrochloric acid (1M, 2 mL) and extracted with ethyl acetate (2 mL×3). The aqueous phase was concentrated under reduced pressure and the residue was purified by prep-HPLC according to one of the purification methods 8-1 or 8-2 to give desired compounds 85 as off-white solids.

### Purification Methods:

PM 8-1: column: Phenomenex Luna C18 75×30 mm×3 μm; mobile phase: [water (0.05% HCl)-ACN]; B %: 10%-35%, 6.5 min.
PM 8-2: column: Phenomenex Luna C18 150×25 mm×10 μm; mobile phase: [water (0.1% TFA)-ACN]; B %: 10%-40%, 10 min.

Following the teachings of the General Reaction Schemes, the coupling methods 8A and 8B and using purification methods 8-1 and 8-2 and the Intermediates disclosed herein, the Examples 8-1 to 8-9 are prepared as shown in Table 8.

TABLE 8

| Example | | | | |
|---|---|---|---|---|
| # | Structure | CM | PM | Compound Name and Characterization |
| 8-1 | 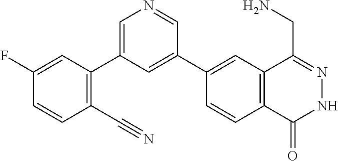 [SMILES: N#Cc1ccc(F)cc1-c1cncc(-c2ccc3c(=O)[nH]nc(CN)c3c2)c1] | 8A | 8-1 | 2-(5-(4-(aminomethyl)-1-oxo-1,2-dihydro- phthalazin-6-yl)pyridin-3-yl)-4-fluoro- benzonitrile; LCMS [M + 1]⁺ = 372.2; ¹H NMR (400 MHz, DMSO-d₆) δ = 13.01 (s, 1H), 9.33 (d, J = 2.0 Hz, 1H), 8.97 (d, J = 2.0 Hz, 1H), 8.68 (t, J = 2.0 Hz, 1H), 8.59 (br s, 3H), 8.42 (m, 3H), 8.18 (dd, J = 5.6, 8.8 Hz, 1H), 7.88 (dd, J = 2.8, 9.6 Hz, 1H), 7.60 (dt, J = 2.8, 8.4 Hz, 1H), 4.59 (br d, J = 5.6 Hz, 2H). |
| 8-2 | 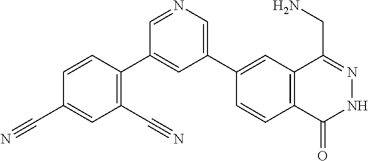 [SMILES: N#Cc1ccc(-c2cncc(-c3ccc4c(=O)[nH]nc(CN)c4c3)c2)c(C#N)c1] | 8B | 8-1 | 4-(5-(4-(aminomethyl)-1-oxo-1,2-dihydro- phthalazin-6-yl)pyridin-3-yl)isophthalo- nitrile LCMS [M + 1]⁺ = 379.0; ¹H NMR (400 MHz, DMSO-d₆) δ = 13.02 (s, 1H), 9.33 (d, J = 2.0 Hz, 1H), 8.97 (d, J = 2.0 Hz, 1H), 8.72 (d, J = 1.6 Hz, 1H), 8.67 (t, J = 2.0 Hz, 1H), 8.52 (br s, 3H), 8.45-8.41 (d, J = 8.4 Hz, 1H), 8.40 (s, 2H), 8.37 (d, J = 1.6 Hz, 1H), 8.10 (d, J = 8.0 Hz, 1H), 4.59 (s, 2H) |
| 8-3 | 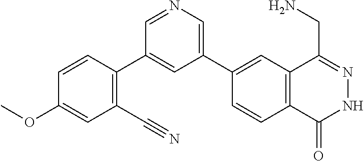 [SMILES: COc1ccc(-c2cncc(-c3ccc4c(=O)[nH]nc(CN)c4c3)c2)c(C#N)c1] | 8A | 8-3 | 2-(5-(4-(aminomethyl)-1-oxo-1,2-dihydro- phthalazin-6-yl)pyridin-3-yl)-5-methoxy- benzonitrile; LCMS [M + 1]⁺ = 384.1; 1H NMR (400 MHz, DMSO-d₆) δ = 13.01 (s, 1H), 9.22 (d, J = 2.0 Hz, 1H), 8.88 (d, J = 2.0 Hz, 1H), 8.53 (t, J = 2.0 Hz, 1H), 8.47- 8.41 (m, 2H), 8.41-8.36 (s, 3H), 8.36-8.34 (m, 1H), 7.78 (d, J = 8.8 Hz, 1H), 7.66 (d, J = 2.8 Hz, 1H), 7.47 (dd, J = 2.8, 8.8 Hz, 1H), 4.61 (br d, J = 2.8 Hz, 2H), 3.90 (s, 3H) |
| 8-4 | 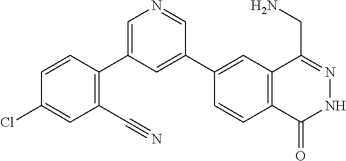 [SMILES: N#Cc1cc(Cl)ccc1-c1cncc(-c2ccc3c(=O)[nH]nc(CN)c3c2)c1] | 8B | 8-1 | 2-(5-(4-(aminomethyl)-1-oxo-1,2-dihydro- phthalazin-6-yl)pyridin-3-yl)-5-chloro- benzonitrile; LCMS [M + 1]⁺ = 388.0: ¹H NMR (400 MHz, DMSO-d₆) δ = 13.01 (s, 1H), 9.33 (d, J = 2.0 Hz, 1H), 8.95 (d, J = 2.0 Hz, 1H), 8.68 (t, J = 2.0 Hz, 1H), 8.60 (br s, 3H), 8.44-8.37 (m, 3H), 8.29 (d, J = 2.0 Hz, 1H), 8.03-7.98 (dd, J = 8.4, 2.4 Hz, 1H), 7.94-7.89 (d, J = 8.4 Hz, 1H), 4.66- 4.49 (d, J = 5.6 Hz, 2H) |
| 8-5 | 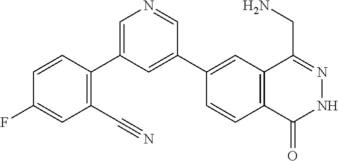 [SMILES: N#Cc1cc(F)ccc1-c1cncc(-c2ccc3c(=O)[nH]nc(CN)c3c2)c1] | 8A | 8-1 | 2-(5-(4-(aminomethyl)-1-oxo-1,2-dihydro- phthalazin-6-yl)pyridin-3-yl)-5-fluoro- benzonitrile; LCMS [M + 1]⁺ = 372.2; ¹H NMR (400 MHz, DMSO-d₆) δ = 13.01 (s, 1H), 9.32 (d, J = 2.0 Hz, 1H), 8.94 (d, J = 2.0 Hz, 1H), 8.68 (t, J = 2.0 Hz, 1H), 8.61 (br s, 3H), 8.44-8.37 (m, 3H), 8.11 (dd, J = 2.8, 8.8 Hz, 1H), 7.95 (dd, J = 5.6, 8.8 Hz, 1H), 7.82 (dt, J = 2.8, 8.8 Hz, 1H), 4.58 (br d, J = 5.6 Hz, 2H). |
| 8-6 | 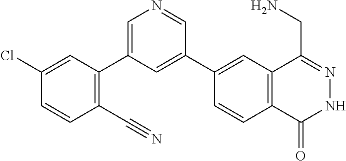 [SMILES: N#Cc1ccc(Cl)cc1-c1cncc(-c2ccc3c(=O)[nH]nc(CN)c3c2)c1] | 8B | 8-3 | 2-(5-(4-(aminomethyl)-1-oxo-1,2-dihydro- phthalazin-6-yl)pyridin-3-yl)-4-chloro- benzonitrile; LCMS [M + 1]⁺ = 388.2; ¹H NMR (400 MHz, DMSO-d₆) δ = 13.01 (s, 1H), 9.33 (d, J = 2.0 Hz, 1H), 8.97 (d, J = 2.0 Hz, 1H), 8.67 (t, J = 2.0 Hz, 1H), 8.58 (br s, 3H), 8.42 (m, 3H), 8.12 (d, J = 8.4 Hz, 1H), 8.06 (d, J = 2.0 Hz, 1H), 7.81 (dd, J = 2.0, 8.4 Hz, 1H), 4.59 (br d, J = 5.6 Hz, 2H) |
| 8-7 | 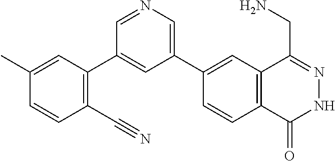 [SMILES: Cc1ccc(C#N)c(-c2cncc(-c3ccc4c(=O)[nH]nc(CN)c4c3)c2)c1] | 8A | 8-3 | 2-(5-(4-(aminomethyl)-1-oxo-1,2-dihydro- phthalazin-6-yl)pyridin-3-yl)-4-methyl- benzonitrile; LCMS [M + 1]⁺ = 368.1; ¹H NMR (400 MHz, MeOD) δ = 9.11 (d, J = 2.0 Hz, 1H), 8.87 (d, J = 2.0 Hz, 1H), 8.62- 8.52 (m, 2H), 8.34 (dd, J = 1.2, 8.4 Hz, 1H), 8.26 (d, J = 1.2 Hz, 1H), 7.83 (d, J = 8.0 Hz, 1H), 7.64 (s, 1H), 7.50 (d, J = 7.6 Hz, 1H), 4.69 (s, 2H), 2.54 (s, 3H) |
| 8-8 | 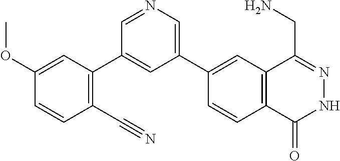 [SMILES: COc1ccc(C#N)c(-c2cncc(-c3ccc4c(=O)[nH]nc(CN)c4c3)c2)c1] | 8A | 8-1 | 2-(5-(4-(aminomethyl)-1-oxo-1,2-dihydro- phthalazin-6-yl)pyridin-3-yl)-4-methoxy- benzonitrile; LCMS [M + 1]⁺ = 384.1; ¹H NMR (400 MHz, DMSO-d₆) δ = 13.01 (s, 1H), 9.29 (d, J = 2.0 Hz, 1H), 8.93 (d, J = 2.0 Hz, 1H), 8.63 (t, J = 2.0 Hz, 1H), 8.59 (br s, 3H), 8.45-8.36 (m, 3H), 7.98 (d, J = 8.8 Hz, 1H), 7.41 (d, J = 2.4 Hz, 1H), 7.24 (dd, J = 2.4, 8.8 Hz, 1H), 4.64 (d, J = 5.6 Hz, 2H), 3.94 (s, 3H) |
| 8-9 | 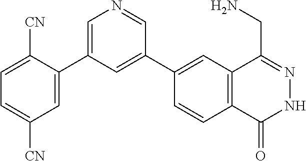 [SMILES: N#Cc1ccc(C#N)c(-c2cncc(-c3ccc4c(=O)[nH]nc(CN)c4c3)c2)c1] | 8B | 8-2 | 2-(5-(4-(aminomethyl)-1-oxo-1,2-dihydro- phthalazin-6-yl)pyridin-3-yl)terephthalo- nitrile LCMS [M + 1]+ = 379.1; ¹H NMR (400 MHz, DMSO-d₆) δ = 13.02 (s, 1H), 9.32 (d, J = 2.0 Hz, 1H), 8.99 (d, J = 2.0 Hz, 1H), 8.65 (t, J = 2.0 Hz, 1H), 8.45 (m, 2H), 8.44-8.40 (m, 4H), 8.38 (s, 1H), 8.31 (d, J = 8.0 Hz, 1H), 8.20 (dd, J = 1.6, 8.0 Hz, 1H), 4.60 (br d, J = 5.2 Hz, 2H) |

### Preparation of Examples 9-1 to 9-3

### Example 9-1

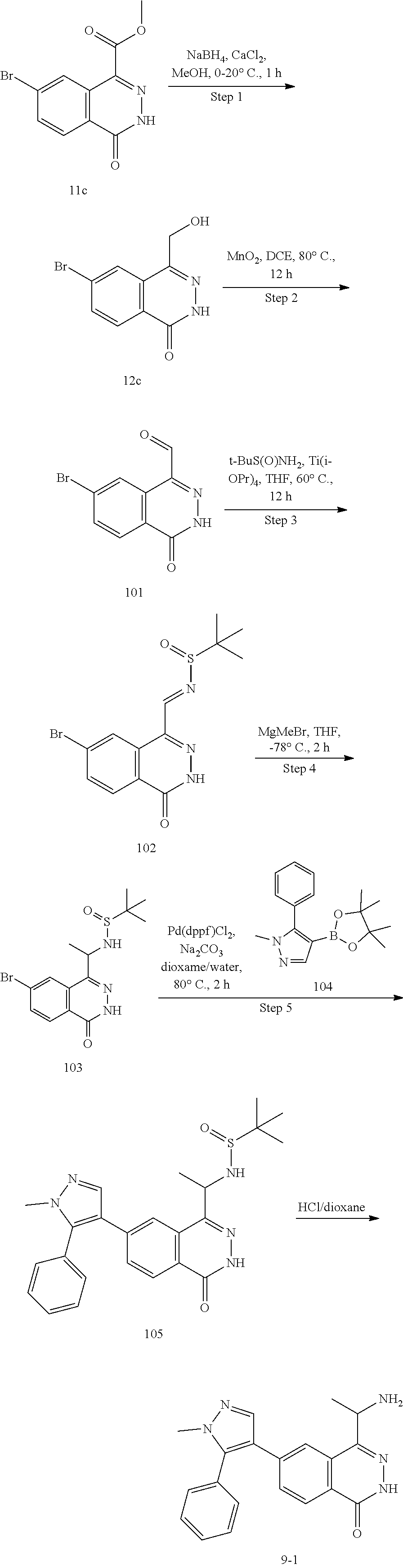

Step 1: A stirred solution of sodium borohydride (382 mg, 10.1 mmol, 2.60 eq.) in ethyl alcohol (150 mL) was treated portion-wise with methyl 7-bromo-4-oxo-3H-phthalazine-1-carboxylate 11c (1.10 g, 3.89 mmol, 1.00 eq.) at 0° C. followed by a dropwise addition of a solution of calcium chloride (518 mg, 4.66 mmol, 1.20 eq.) in ethyl alcohol (150 mL). Stirring was continued for additional 3 hours at the 0° C., and then 1 hour at 20° C. After such time the reaction mixture was concentrated under reduced pressure and the residue was diluted with water (30 mL) and adjusted to pH 5 with 1N hydrochloric acid (5 mL). The resulting solid was filtered and washed with water (5 mL×3) and then triturated with ethyl alcohol (20 mL) to afford 6-bromo-4-(hydroxymethyl)-2H-phthalazin-1-one 12c (990 mg, 3.88 mmol, 100% yield) as a white solid.

Step 2: To a solution of 6-bromo-4-(hydroxymethyl)-2H-phthalazin-1-one 12c (300 mg, 1.18 mmol, 1.00 eq.) in DCE (10 mL) was added manganese dioxide (1.02 g, 11.8 mmol, 10.0 eq.) and the mixture was stirred at 80° C. for 12 hours. After such time the reaction mixture was filtered, and the filtrate concentrated under reduced pressure and the residue was purified by column chromatography (SiO₂, petroleum ether:ethyl acetate 10:1 to 1:1) to give 7-bromo-4-oxo-3H-phthalazine-1-carbaldehyde 101 (73 mg, 288 μmol, 25% yield) as a white solid. ¹H NMR (400 MHz, DMSO-d₆) δ=13.66 (br s, 1H), 9.82 (s, 1H), 9.05 (d, J=2.0 Hz, 1H), 8.22-8.18 (d, J=8.4 Hz, 1H), 8.10 (dd, J=2.0, 8.4 Hz, 1H).

Step 3: To a solution of 7-bromo-4-oxo-3H-phthalazine-1-carbaldehyde 101 (300 mg, 1.19 mmol, 1.00 eq.) in THF (50 mL) was added titanium iso-propoxide (674 mg, 2.37 mmol, 700 μL, 2.00 eq.) and 2-methylpropane-2-sulfinamide (216 mg, 1.78 mmol, 1.50 eq.) and the mixture was stirred at 60° C. for 12 hours. After such time the reaction mixture was quenched with water (1 mL) and the resulting solid was filtered. The filtrate was dried over sodium sulfate, filtered and concentrated and the residue was purified by column chromatography (SiO₂, petroleum ether/ethyl acetate=5/1 to 1/1) to give N-[(7-bromo-4-oxo-3H-phthalazin-1-yl)methylene]-2-methyl-propane-2-sulfinamide 102 (100 mg, 171 μmol, 14% yield) as a white solid. LCMS [M+1]⁺=358.1. ¹H NMR (400 MHz, DMSO-d₆) δ=13.58 (br s, 1H), 9.32 (d, J=2.0 Hz, 1H), 8.37 (s, 1H), 8.23 (d, J=8.4 Hz, 1H), 8.12-8.10 (dd, J=2.0, 8.4 Hz, 1H), 1.26 (s, 9H).

Step 4: To a solution of N-[(7-bromo-4-oxo-3H-phthalazin-1-yl)methylene]-2-methyl-propane-2-sulfinamide 102 (100 mg, 281 μmol, 1.00 eq.) in THF (5 mL) was added methylmagnesium bromide (3M, 281 μL, 3.00 eq.) dropwise at −78° C. The reaction mixture was then stirred at −78° C. for 2 hours. After such time the reaction mixture was diluted with water (2 mL) and extracted with ethyl acetate (2 mL×3). The combined organic portions were washed with brine (2 mL), dried over anhydrous sodium sulfate, filtered and concentrated and the residue was purified by prep-TLC (petroleum ether/ethyl acetate=10/1) to give N-[1-(7-bromo-4-oxo-3H-phthalazin-1-yl)ethyl]-2-methyl-propane-2-sulfinamide 103 (40 mg, 98.2 μmol, 35% yield) as a yellow solid. LCMS [M+1]⁺=374.2.

Step 5: A mixture of N-[1-(7-bromo-4-oxo-3H-phthalazin-1-yl)ethyl]-2-methyl-propane-2-sulfinamide 103 (35 mg, 94 μmol, 1.00 eq.), 1-methyl-5-phenyl-4-(4,4,5,5-tetramethyl-1,3,2-dioxaborolan-2-yl)pyrazole 104 (35 mg, 122 μmol, 1.30 eq.), Pd(dppf)Cl₃ (7 mg, 9 μmol, 0.10 eq.), sodium carbonate (20 mg, 188 μmol, 2.00 eq.) in water (0.2 mL) and dioxane (1 mL) was degassed and purged with nitrogen 3 times, and then stirred at 80° C. for 2 hours. After such time the reaction mixture was diluted with water (2 mL) and extracted with ethyl acetate (2 mL×3) and the combined organic phases were washed with brine (2 mL), dried over anhydrous sodium sulfate, filtered and concentrated and the residue was purified by prep-TLC (petroleum ether:ethyl acetate 1:1) to give 2-methyl-N-[1-[7-(1-methyl-5-phenyl-pyrazol-4-yl)-4-oxo-3H-phthalazin-1-yl]ethyl]propane-2-sulfinamide 105 (40 mg, 60 μmol, 63% yield) as a brown oil. LCMS [M+1]⁺=450.4.

Step 6: A mixture of 2-methyl-N-[1-[7-(1-methyl-5-phenyl-pyrazol-4-yl)-4-oxo-3H-phthalazin-1-yl]ethyl]propane-2-sulfinamide 105 (36 mg, 80 μmol, 1.00 eq.) and hydrochloric acid/dioxane (4M, 9 mL) was stirred at 25° C. for 1 hour. The reaction mixture was concentrated under reduced pressure and the residue was purified by prep-HPLC (Phenomenex Luna C18 75×30 mm×3 μm; mobile phase: [water (0.05% hydrochloric acid)-ACN]; B %: 12/0-32%, 6.5 min) to give 4-(1-aminoethyl)-6-(1-methyl-5-phenyl-pyrazol-4-yl)-2H-phthalazin-1-one, Example 9-1 (7 mg, 19 μmol, 23% yield, HCl) as a white solid. LCMS [M+1]⁺=346.1. ¹H NMR (400 MHz, MeOD) δ=8.30 (d, J=8.4 Hz, 1H), 8.13 (s, 1H), 7.91 (dd, J=1.6, 8.4 Hz, 1H), 7.66-7.60 (m, 3H), 7.55 (d, J=1.6 Hz, 1H), 7.50-7.45 (m, 2H), 4.66-4.53 (m, 1H), 3.81 (s, 3H), 1.32 (d, J=6.8 Hz, 3H).

### Examples 9-2 & 9-3

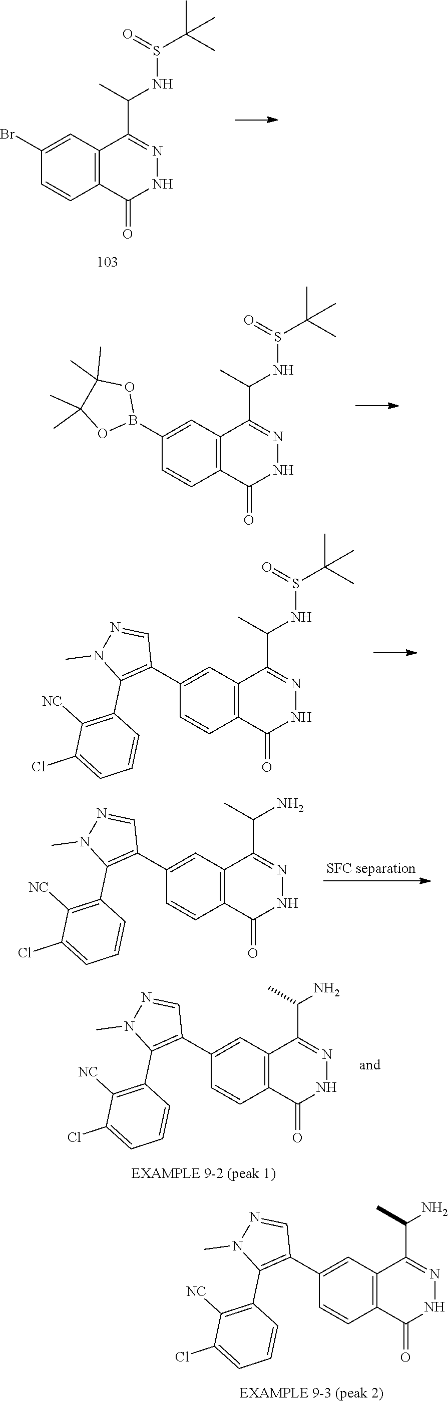

Step 1: A mixture of (R)—N-(1-(7-bromo-4-oxo-3,4-dihydrophthalazin-1-yl)ethyl)-2-methylpropane-2-sulfinamide 103 (410 mg, 1.10 mmol, 1.00 eq.), 4,4,5,5-tetramethyl-2-(4,4,5,5-tetramethyl-1,3,2-dioxaborolan-2-yl)-1,3,2-dioxaborolane (839 mg, 3.30 mmol, 3.00 eq.), Pd(dppf)Cl₂ (81 mg, 0.11 mmol, 0.10 eq.) and potassium acetate (324 mg, 3.30 mmol, 3.00 eq.) in dioxane (10 mL) was degassed and purged with nitrogen for 3 times, and then the mixture was stirred at 100° C. for 0.5 hour under nitrogen atmosphere. The reaction mixture was concentrated under reduced pressure and the residue was triturated with petroleum ether (40 mL) to give (R)-2-methyl-N-(1-(4-oxo-7-(4,4,5,5-tetramethyl-1,3,2-dioxaborolan-2-yl)-3,4-dihydrophthalazin-1-yl)ethyl)propane-2-sulfinamide (724 mg, crude) as a gray solid which used into the next step without further purification. LCMS [M−81]⁻=338.2.

Step 2: A mixture of (R)-2-methyl-N-(1-(4-oxo-7-(4,4,5,5-tetramethyl-1,3,2-dioxaborolan-2-yl)-3,4-dihydrophthalazin-1-yl)ethyl)propane-2-sulfinamide (670 mg, 1.60 mmol, 1.00 eq.), 2-(4-bromo-2-methyl-pyrazol-3-yl)-6-chloro-benzonitrile (308. mg, 1.04 mmol, 0.65 eq.), di-tert-butyl(cyclopentyl)phosphane;dichloropalladium;iron (104 mg, 0.160 mmol, 0.10 eq.), sodium carbonate (339 mg, 3.20 mmol, 2.00 eq.) in dioxane (10 mL) and water (2 mL) was degassed and purged with nitrogen for 3 times, and then the mixture was stirred at 80° C. for 0.5 hour under nitrogen atmosphere. The reaction mixture was diluted with water (200 mL) and extracted with ethyl acetate (150 mL×3). The combined organic layers were washed with brine (100 mL), dried over anhydrous sodium sulfate, filtered and concentrated under reduced pressure and the residue was purified by column chromatography (SiO₂, petroleum ether:ethyl acetate=1:1 to 0:1, dichloromethane:methyl alcohol=10:1) to give (R)—N-(1-(7-(5-(3-chloro-2-cyanophenyl)-1-methyl-1H-pyrazol-4-yl)-4-oxo-3,4-dihydrophthalazin-1-yl)ethyl)-2-methylpropane-2-sulfinamide (281 mg, 0.552 mmol, 34% yield) as a brown solid. LCMS [M+1]⁺=509.2.

Step 3: To a solution of (R)—N-(1-(7-(5-(3-chloro-2-cyanophenyl)-1-methyl-1H-pyrazol-4-yl)-4-oxo-3,4-dihydrophthalazin-1-yl)ethyl)-2-methylpropane-2-sulfinamide (140 mg, 0.275 mmol, 1.00 eq.) in hydrochloride/dioxane (4.00 M, 5.4 mL) and the mixture was stirred at 15° C. for 0.5 hour. The reaction mixture was concentrated under reduced pressure and the residue was purified by prep-HPLC (Phenomenex Luna C18 75×30 mm×3 μm; mobile phase: [water (0.05% HCl)-ACN]; B %: 12%-32%, 7 min) to give 2-[4-[4-(1-aminoethyl)-1-oxo-2H-phthalazin-6-yl]-2-methyl-pyrazol-3-yl]-6-chloro-benzonitrile (40 mg, 0.097 mmol, 36% yield) as a yellow solid. LCMS [M+1]⁺=405. 1; ¹H NMR (400 MHz, CD₃OD) δ=8.33-8.23 (m, 1H), 8.13 (d, J=4.4 Hz, 1H), 8.02-7.84 (m, 2H), 7.82-7.66 (m, 2H), 7.64-7.57 (m, 1H), 4.87 (br s, 1H), 3.83 (d, J=2.8 Hz, 3H), 1.54-1.41 (m, 3H).

Step 4: Enantiomers were separated by chiral SFC (DAICEL CHIRALPAK IC (250 mm×30 mm, 10 μm); mobile phase: [0.1% NH₄OH/EtOH]; B %: 50%-50%, 4.2 min; 46 min) followed by chiral SFC Chiralpak IC-3 (50×4.6 mm I.D., 3 μm); Mobile phase: Phase A for CO₂, and Phase B for EtOH (0.05% DEA); Gradient elution: 50% EtOH (0.05% DEA) in CO₂ Flow rate: 3 mL/min; Detector: PDA; Column Temp: 35° C.; Back Pressure: 100 Bar to give peak 1, Example 9-2, (11 mg, 0.025 mmol, 30% yield) as a yellow solid and peak 2, Example 9-3 (18 mg, 0.444 mmol, 50% yield) as an off-white solid. Each example was converted to the HCl salt. Example 9-2, Peak 1, single enantiomer, stereochemistry unassigned: LCMS [M+1]⁺=405.1; ¹H NMR (400 MHz, CD₃OD) δ=8.31 (t, J=8.4 Hz, 1H), 8.12 (d, J=6.4 Hz, 1H), 7.92-7.87 (m, 2H), 7.80-7.67 (m, 3H), 4.88-4.84 (m, 1H), 3.83 (d, J=1.2 Hz, 3H), 1.51-1.43 (m, 3H). Example 9-3, Peak 2, single enantiomer, stereochemistry unassigned: LCMS [M+1]⁺=405.1; ¹H NMR (400 MHz, CD₃OD) δ=8.29 (dd, J=8.2, 10.4 Hz, 1H), 8.13 (d, J=7.6 Hz, 1H), 7.90-7.85 (m, 2H), 7.79-7.60 (m, 3H), 4.91-4.85 (m, 1H), 3.82 (d, J=2.8 Hz, 3H), 1.52-1.41 (m, 3H).

### Examples 9-4 & 9-5

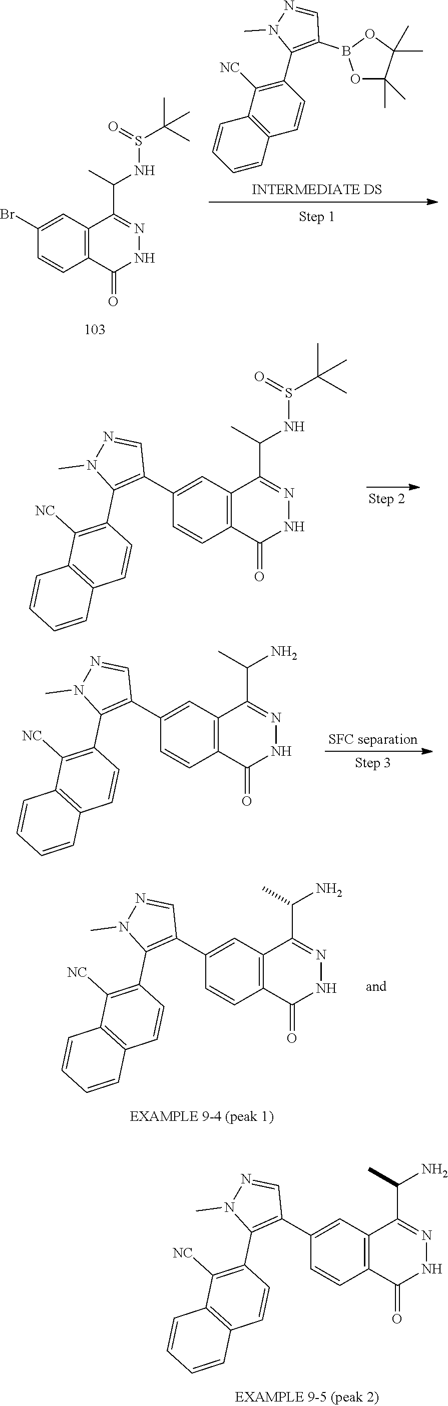

Step 1: A mixture of 2-[2-methyl-4-(4,4,5,5-tetramethyl-1,3,2-dioxaborolan-2-yl)pyrazol-3-yl]naphthalene-1-carbonitrile (80 mg, 0.222 mmol, 1.00 eq.), N-[1-(7-bromo-4-oxo-3H-phthalazin-1-yl)ethyl]-2-methyl-propane-2-sulfinamid0.e (100 mg, 0.267 mmol, 1.20 eq.), di-tert-butyl(cyclopentyl)phosphane-dichloropalladium-iron (29 mg, 0.044 mmol, 0.20 eq.), potassium phosphate (142 mg, 0.668 mmol, 3.00 eg.) in dioxane (4 mL) and water (0.8 mL) and was degassed and purged with nitrogen and then stirred at 100° C. for 1 hour. After such time the reaction mixture was concentrated under reduced pressure and the residue was purified by prep-HPLC (Phenomenex Synergi C18 150×30 mm×4 μm; mobile phase: [water (0.1% TA)-ACN]; B %: 35%-64%, 11 mi) to give N-[1-[7-[5-(1-cyano-2-naphthyl)-1-methyl-pyrazol-4-yl]-4-oxo-3H-phthalazin-1-yl]ethyl]-2-methyl-propane-2-sulfinamide (48 mg, 0.091 mmol, 41% yield) was obtained as a white solid. LCMS [M+1]⁺=525.2.

Step 2: A mixture of N-[a-[7-[5-(1-cyano-2-naphthyl)-1-methyl-pyrazol-4-yl]-4-oxo-3H-phthalazin-1-yl]ethyl]-2-methyl-propane-2-sulfinamide (40. mg, 0.076 mmol, 1.00 eq.) in HCl/dioxane (1 mL, 4 M) and methanol (1 mL) was stirred at 20° C. for 1 hour. The reaction mixture was the concentrated to give 2-[4-[4-(1-aminoethyl)-1-oxo-2H-phthalazin-6-yl]-2-methyl-pyrazol-3-yl]naphthalene-1-carbonitrile (30 mg, crude) as a white solid which used into the next step without further purification. LCMS [M+1]⁺=421.2.

Step 3: SFC purification, Chiralpak IG-3 50 Á 4.6 mm, 3 μm Mobile phase: A: CO₂ B: ethanol (0.05% DEA) Isocratic: 40% B. Flow rate: 4 mL/min. Column temp. 35° C. ABPR 1500 psi gave peak 1, Example 9-4 2-[4-[4-(1-aminoethyl)-1-oxo-2H-phthalazin-6-yl]-2-methyl-pyrazol-3-yl]naphthalene-1-carbonitrile (12 mg, 29 μmol, 33% yield) as a white solid ¹H NMR (400 MHz, DMSO-d₆) δ=12.42 (br s, 1H), 8.59-8.50 (m, 1H), 8.30-8.22 (m, 2H), 8.20-8.09 (m, 2H), 7.78-7.54 (m, 5H), 3.95-3.70 (m, 4H), 0.50-0.80 (m, 3H). And peak 2, Example 9-5 and 2-[4-[4-(1-aminoethyl)-1-oxo-2H-phthalazin-6-yl]-2-methyl-pyrazol-3-yl]naphthalene-1-carbonitrile (12 mg, 29 μmol, 33% yield) as a white solid ¹H NMR (400 MHz, DMSO-d₆) δ=12.41 (br s, 1H), 8.60-8.50 (m, 1H), 8.30-8.23 (m, 2H), 8.19-8.09 (m, 2H), 7.92-7.54 (m, 6H), 3.90-3.70 (m, 4H), 0.83-0.55 (m, 3H).

### Examples 9-6 & 9-7

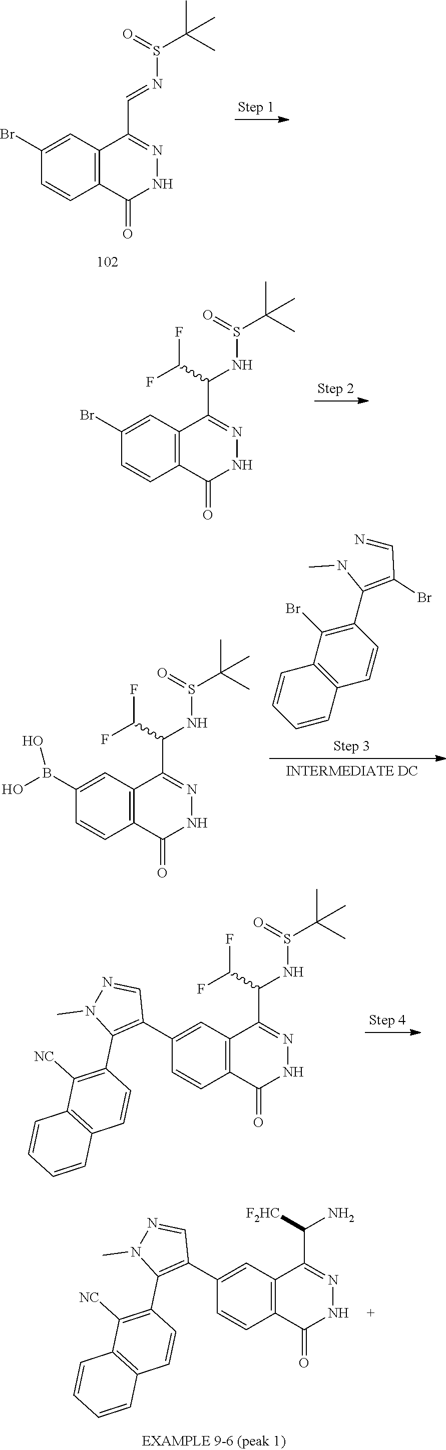

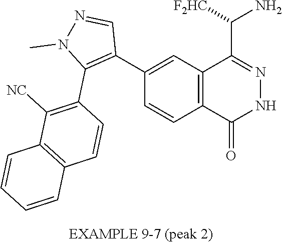 [SMILES: Cn1ncc(-c2ccc3c(=O)[nH]nc([C@@H](N)C(F)F)c3c2)c1-c1ccc2ccccc2c1C#N]

Step 1: To a solution of N-[(7-bromo-4-oxo-3H-phthalazin-1-yl)methylene]-2-methyl-propane-2-sulfinamide (276 mg, 0.775 mmol, 1.00 eq.) and difluoromethyl(trimethyl)silane (289 mg, 2.32 mmol, 3.00 eq.) in THF (2.0 mL) was added potassium tert-butoxide (1 M, 2.3 mL, 3.00 eq.) and the mixture was stirred at −78° C. for 12 hours. The reaction mixture was then quenched with water (2 mL) and extracted with ethyl acetate (2 mL×3). The combined organic phase were then washed with brine (2 mL), dried over anhydrous sodium sulfate, filtered, concentrated and the residue was purified by prep-TLC (SiO₂, dichloromethane:methyl alcohol 10:1) to give N-[1-(7-bromo-4-oxo-3H-phthalazin-1-yl)-2,2-difluoro-ethyl]-2-methyl-propane-2-sulfinamide (107 mg, 0.182 mmol, 24% yield) as a black solid. LCMS [M+]⁺=410.1; ¹H NMR (400 MHz, CDCl₃-d) δ=10.58-10.26 (m, 1H), 8.29-8.22 (m, 1H), 8.06 (s, 1H), 7.93-7.86 (m, 1H), 6.22-5.86 (m, 1H), 4.72 (d, J=7.2 Hz, 1H), 1.30 (s, 9H).

Step 2: A mixture of N-[1-(7-bromo-4-oxo-3H-phthalazin-1-yl)-2,2-difluoro-ethyl]-2-methyl-propane-2-sulfinamide (97 mg, 0.238 mmol, 1.00 eq.), bis(pinacolato)diboron (78 mg, 0.309 mmol, 1.30 eq.), Pd(dppf)Cl₂ (17 mg, 0.024 mmol, 0.10 eq.), potassium acetate (67 mg, 0.713 mmol, 3.00 eq.) in dioxane (1.0 mL) was degassed and purged with nitrogen 3 times, and then stirred at 100° C. for 1 hour. The mixture was then concentrated under reduced pressure to give [4-[1-(tert-butylsulfinylamino)-2,2-difluoro-ethyl]-1-oxo-2H-phthalazin-6-yl]boronic acid (100 mg, crude) as a black solid. LCMS [M+1]⁺=374.1.

Step 3: A mixture of [4-[1-(tert-butylsulfinylamino)-2,2-difluoro-ethyl]-1-oxo-2H-phthalazin-6-yl]boronic acid (100 mg, crude), 2-(4-bromo-2-methyl-pyrazol-3-yl)naphthalene-1-carbonitrile (77 mg, 0.247 mmol), sodium bicarbonate (62 mg, 0.740 mmol), di-tert-butyl(cyclopentyl)phosphane;dichloropalladium;iron (16 mg, 0.024 mmol, 0.10 eq.) in dioxane (3.0 mL) and water (0.6 mL) was degassed and purged with nitrogen 3 times, and then stirred at 100° C. for 2 hours under nitrogen atmosphere. The mixture was then concentrated under reduced pressure and the residue was purified by prep-TLC (dichloromethane:methyl alcohol 10:1) to give N-[1-[7-[5-(1-cyano-2-naphthyl)-1-methyl-pyrazol-4-yl]-4-oxo-3H-phthalazin-1-yl]-2,2-difluoro-ethyl]-2-methyl-propane-2-sulfinamide (36 mg, 0.037 mmol, 15% yield) as a yellow solid. LCMS [M+1]⁺=561.2.

Step 4: A mixture of N-[1-[7-[5-(1-cyano-2-naphthyl)-1-methyl-pyrazol-4-yl]-4-oxo-3H-phthalazin-1-yl]-2,2-difluoro-ethyl]-2-methyl-propane-2-sulfinamide (30 mg, 0.053 mmol, 1.00 eq.) in hydrochloric acid/dioxane (1.0 mL) was degassed and purged with nitrogen 3 times, and the mixture stirred at 0° C. for 2 hours. The mixture was then concentrated and the residue purified by prep-HPLC (Phenomenex Luna C18 75×30 mm×3 μm; mobile phase: [water (0.05% hydrochloric acid)-ACN]; B %: 18%-38%, 7 min) then further separated by SFC (Daicel ChiralPak IG (250×30 mm, 10 μm); mobile phase: [Mobile phase: A: CO₂ B: ACN/EtOH (0.1% NH₃·water)] isocratic 60/6, 50 min) to give Example 9-6, 2-[4-[4-[(1S)-1-amino-2,2-difluoro-ethyl]-1-oxo-2H-phthalazin-6-yl]-2-methyl-pyrazol-3-yl]naphthalene-1-carbonitrile (5.2 mg, 0.011 mmol, 21% yield) as a brown solid and Example 9-7, 2-[4-[4-[(1R)-1-amino-2,2-difluoro-ethyl]-1-oxo-2H-phthalazin-6-yl]-2-methyl-pyrazol-3-yl]naphthalene-1-carbonitrile (4.3 mg, 0.009 mmol, 17% yield) as a brown solid. Example 9-6: LCMS [M+1]⁺=457.1; ¹H NMR (400 MHz, DMSO-d₆) δ=12.61 (br d, J=3.6 Hz, 1H), 8.53 (dd, J=8.8, 10.4 Hz, 1H), 8.29 (s, 1H), 8.27 (d, J=7.6 Hz, 1H), 8.14 (dd, J=8.2, 14.4 Hz, 1H), 8.07 (dd, J=6.8, 8.4 Hz, 1H), 7.92-7.74 (m, 4H), 7.63 (ddd, J=1.6, 8.4, 17.2 Hz, 1H), 6.14-5.59 (m, 1H), 4.17-3.95 (m, 1H), 3.78 (d, J=1.6 Hz, 3H), 3.31 (br s, 2H). ¹H NMR (400 MHz, MeOD) 5=8.43 (dd, J=8.4, 18.0 Hz, 1H), 8.30-8.11 (m, 4H), 7.89-7.61 (m, 5H), 5.95-5.29 (m, 1H), 4.13-3.98 (m, 1H), 3.84 (d, J=2.4 Hz, 3H). Example 9-7: LCMS [M+1]⁺=457.0; ¹H NMR (400 MHz, DMSO-d₆) δ=12.71-12.36 (m, 1H), 8.58-8.48 (m, 1H), 8.29 (s, 1H), 8.27 (d, J=7.6 Hz, 1H), 8.14 (dd, J=8.0, 14.4 Hz, 1H), 8.07 (dd, J=6.8, 8.4 Hz, 1H), 7.93-7.74 (m, 4H), 7.63 (ddd, J=1.6, 8.4, 17.2 Hz, 1H), 6.10-5.59 (m, 1H), 4.10 (q, J=5.6 Hz, 1H), 3.78 (d, J=1.6 Hz, 3H), 3.31 (br s, 2H). ¹H NMR (400 MHz, MeOD) δ=8.44 (dd, J=8.4, 17.6 Hz, 1H), 8.29-8.12 (m, 4H), 7.90-7.62 (m, 5H), 5.94-5.36 (m, 1H), 4.12-4.06 (m, 1H), 3.85 (d, J=2.4 Hz, 3H).

### Example 9-8

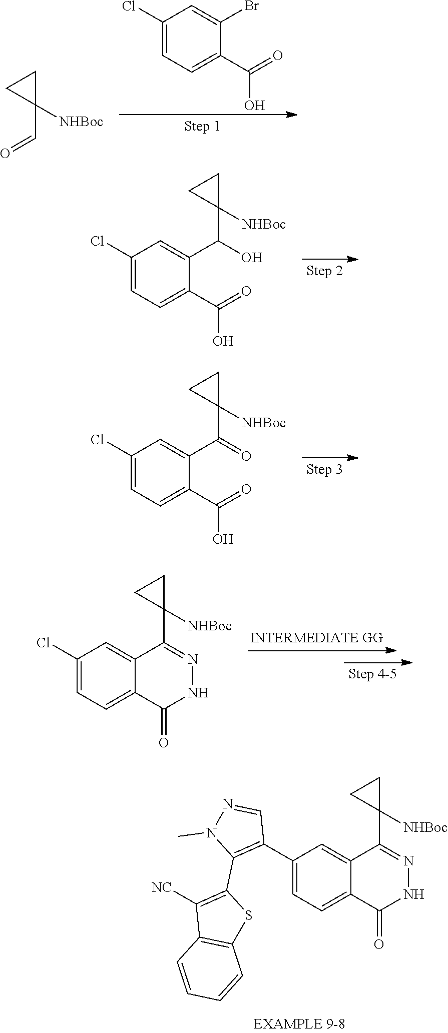

Step 1: To a solution of 2-bromo-4-chloro-benzoic acid (3.80 g, 16.1 mmol, 1.20 eq.) in 3,4-dimethylTHF (5.0 mL) was added dropwise n-butyllithium (2.50 M, 13.5 mL, 2.50 eq.) at −70° C. After addition, the mixture was stirred at this temperature for 30 min before tert-butyl N-(1-formylcyclopropyl)carbamate (2.50 g, 13.5 mmol, 1.00 eq.) in 3,4-dimethylTHF (5.0 mL) was added dropwise at −70° C. The resulting mixture was stirred at −70° C. for 2 hours followed by stirring at 20° C. for 12 hours. The reaction was quenched with saturated ammonium chloride (20 mL). The mixture was extracted with ethyl acetate (3×50 mL). The combined organic extracts were washed with saturated ammonium chloride (30 mL), brine (30 mL), dried over sodium sulfate, filtered and concentrated in vacuum. The residue was purified by prep-HPLC (column: 3_Phenomenex Luna C18 75×30 mm×3 μm; mobile phase: [water (0.225% FA)-ACN]; B %: 25%-55%, 10 min) to give 2-[[1-(tert-butoxycarbonylamino) cyclopropyl]-hydroxy-methyl]-4-chloro-benzoic acid (340 mg, 6% yield) as a white solid. ¹H NMR (400 MHz, DMSO-d₆) δ=12.97 (s, 1H), 7.74-7.70 (m, 1H), 7.68-7.53 (m, 2H), 7.43-7.32 (m, 1H), 6.94 (m, 1H), 5.46 (d, J=3.2 Hz, 1H), 1.29 (s, 9H), 1.05-0.94 (m, 2H), 0.92-0.83 (m, 2H).

Step 2: To a solution of 2-[[1-(tert-butoxycarbonylamino)cyclopropyl]-hydroxy-methyl]-4-chloro-benzoic acid (300 mg, 877 μmol, 1.00 eq.) in dichloromethane (5.0 mL) was added Dess-Martin periodinane (745 mg, 1.76 mmol, 544 μL, 2.00 eq.). The mixture was stirred at 20° C. for 12 hours. The reaction was quenched with saturated sodium sulfite (20 mL). The mixture was extracted with ethyl acetate (3×30 mL). The combined organic extracts were washed with saturated sodium bicarbonate (20 mL) and brine (20 mL) and then dried over sodium sulfate. The residue was purified by column chromatography on silica gel (petroleum ether/Ethyl acetate 5:1 (0.1% of formic acid))] to give 2-[1-(tert-butoxycarbonylamino) cyclopropanecarbonyl]-4-chloro-benzoic acid (120 mg, 38% yield) as a white solid. ¹H NMR (400 MHz, DMSO-d₆) δ=13.35 (s, 1H), 7.89 (d, J=8.4 Hz, 1H), 7.63 (s, 1H), 7.57 (dd, J=2.0, 8.4 Hz, 1H), 7.26 (d, J=2.0 Hz, 1H), 1.55 (q, J=4.0 Hz, 2H), 1.31-1.25 (m, 2H), 1.20-1.13 (m, 9H).

Step 3: To a solution of 2-[1-(tert-butoxycarbonylamino)cyclopropanecarbonyl]-4-chloro-benzoic acid (120 mg, 353 μmol, 1.00 eq.) in ethanol (2.0 mL) was added hydrazine hydrate (88.4 mg, 1.77 mmol, 85.8 μL, 5.00 eq.). The mixture was stirred at 78° C. for 12 hours. The reaction mixture was filtered. The solid was dried under reduced pressure to give tert-butyl N-[1-(7-chloro-4-oxo-3H-phthalazin-1-yl)cyclopropyl]carbamate (70 mg, 58% yield) as a white solid. ¹H NMR (400 MHz, DMSO-d₆) δ=12.60 (s, 1H), 8.62-8.49 (m, 1H), 8.27-8.22 (m, 1H), 8.10-7.93 (m, 1H), 7.90-7.86 (m, 1H), 1.29 (s, 9H), 1.24-1.22 (m, 2H), 1.14-1.09 (m, 2H).

Step 4: A mixture of tert-butyl N-[1-(7-chloro-4-oxo-3H-phthalazin-1-yl)cyclopropyl]carbamate (50.0 mg, 149 μmol, 1.00 eq.), Intermediate GG (163 mg, 447 μmol, 3.00 eq.), cesium carbonate (97.0 mg, 298 μmol, 2.00 eq.), SPhos-Pd-G2 (10.7 mg, 14.9 μmol, 0.10 eq.) and water (0.20 mL) in dioxane (2.0 mL) was degassed and stirred at 80° C. for 3 hours under nitrogen atmosphere. After being cooled to room temperature, the mixture was concentrated under vacuum. The residue was purified by column chromatography on silica gel (petroleum ether/ethyl acetate 1:2) to give tert-butyl N-[1-[7-[5-(3-cyanobenzothiophen-2-yl)-1-methyl-pyrazol-4-yl]-4-oxo-3H-phthalazin-1-yl]cyclopropyl]carbamate (33 mg, 40% yield) as a yellow solid. LCMS [M+1]⁺=539.3

Step 5: To a solution of tert-butyl N-[1-[7-[5-(3-cyanobenzothiophen-2-yl)-1-methyl-pyrazol-4-yl]-4-oxo-3H-phthalazin-1-yl] cyclopropyl]carbamate (33.0 mg, 61.3 μmol, 1.00 eq.) in dichloromethane (10.0 mL) was added TFA (1.54 g, 13.5 mmol, 1.0 mL, 220 eq.). The mixture was stirred at 20° C. for 1 hour before being concentrated under vacuum. The residue was purified by prep-HPLC (column: Welch Xtimate C18 150×25 mm×5 μm; mobile phase: [water (0.05% HCl)-ACN]; B %: 8%-38%, 10 min) to give 2-(4-(4-(1-aminocyclopropyl)-1-oxo-1, 2-dihydrophthalazin-6-yl)-1-methyl-1H-pyrazol-5-yl)benzo[b]thiophene-3-carbonitrile (9 mg, 35% yield) as a white solid. LCMS [M+1]⁺=439.1. ¹H NMR (400 MHz, MeOD-d₄) δ=8.34 (d, J=8.4 Hz, 1H), 8.16 (s, 1H), 8.15-8.11 (m, 1H), 8.05-8.00 (m, 1H), 7.93 (d, J=1.2 Hz, 1H), 7.88 (dd, J=1.6, 8.4 Hz, 1H), 7.73-7.63 (m, 2H), 4.01 (s, 3H), 1.10-1.03 (m, 2H), 1.02-0.96 (m, 2H).

### Example 9-11

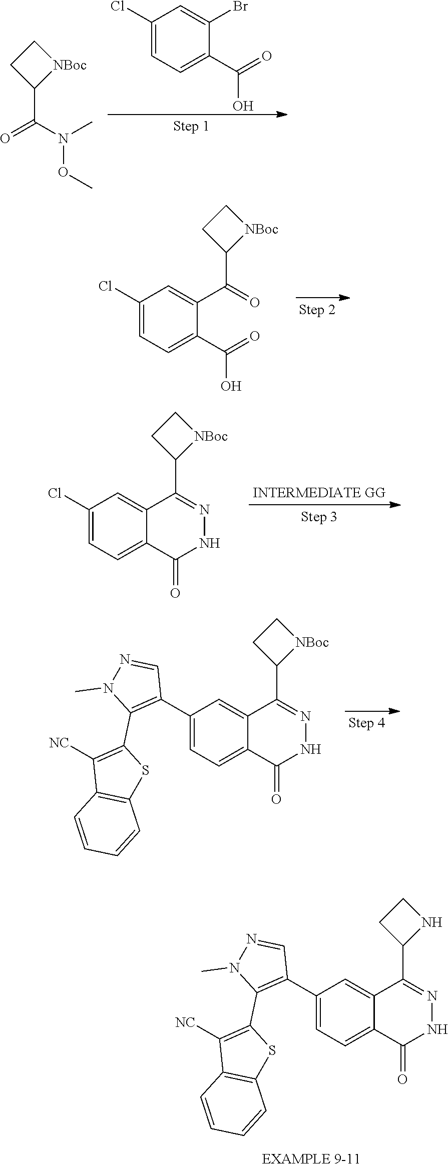 [SMILES: CON(C)C(=O)C1CCN1C(=O)OC(C)(C)C ; O=C(O)c1ccc(Cl)cc1Br ; CC(C)(C)OC(=O)N1CCC1C(=O)c1cc(Cl)ccc1C(=O)O ; CC(C)(C)OC(=O)N1CCC1c1n[nH]c(=O)c2ccc(Cl)cc12 ; Cn1ncc(-c2ccc3c(=O)[nH]nc(C4CCN4C(=O)OC(C)(C)C)c3c2)c1-c1sc2ccccc2c1C#N ; Cn1ncc(-c2ccc3c(=O)[nH]nc(C4CCN4)c3c2)c1-c1sc2ccccc2c1C#N]

Step 1: To a solution of 2-bromo-4-chlorobenzoic acid (530 mg, 2.25 mmol, 1.10 eq.) in 2-methyl THF (5.0 mL) was added N-butyllithium (2.50 M, 2.46 mL, 3.00 eq.) drop-wise at −60° C. and stirred for 1 hour, then tert-butyl 2-(methoxy(methyl)carbamoyl)azetidine-1-carboxylate (500 mg, 2.05 mmol, 1.00 eq) was added and the mixture was stirred at −60° C. for 1 hour. After that, the solution was warmed up to 25° C. and stirred for 1 hour. After completion, the mixture was diluted with saturated ammonium chloride aqueous solution (20 mL), extracted with ethyl acetate (20 mL×3), dried with anhydrous sodium sulfate and filtered. The filtrate was concentrated under reduced pressure and the residue was purified by prep-TLC (silica gel plate, petroleum ether/ethyl acetate 1:1) to give 2-(1-tert-butoxycarbonyl)azetidine-2-carbonyl)-4-chlorobenzoic acid (120 mg, 18% yield) as a yellow oil. ¹H NMR (400 MHz, DMSO-d₆) δ=7.81 (d, J=8.0 Hz, 1H), 7.78-7.72 (m, 1H), 7.69 (d, J=8.4 Hz, 1H), 4.68-4.58 (m, 1H), 3.75-3.62 (m, 2H), 2.69-2.61 (m, 1H), 2.36-2.28 (m, 1H), 1.22-1.05 (m, 9H).

Step 2: To a solution of 2-(1-(tert-butoxycarbonyl)azetidine-2-carbonyl)-4-chlorobenzoic acid (120 mg, 353 μmol, 1.00 eq.) in ethanol (2.0 mL) was added hydrazine hydrate (90.2 mg, 1.77 mmol, 87.6 μL, 98.0% purity, 5.00 eq.). The mixture was stirred at 90° C. for 2 hours under nitrogen atmosphere. After completion of reaction, the mixture was concentrated under reduced pressure to dryness. The residue was purified by prep-TLC (silica gel, petroleum ether/ethyl acetate 1:1) to give tert-butyl 2-(7-chloro-4-oxo-3,4-dihydrophthalazin-1-yl)azetidine-1-carboxylate (83 mg, 69% yield) as a white solid. LCMS [M+1]⁺: 336.1

Step 3: To a solution of tert-butyl 2-(7-chloro-4-oxo-3,4-dihydrophthalazin-1-yl)azetidine-1-carboxylate (70.0 mg, 208 μmol, 1.00 eq.) and Intermediate GG (228 mg, 625 μmol, 3.00 eq.) in dioxane (1.0 mL) was added water (0.30 mL), cesium carbonate (204 mg, 625 μmol, 3.00 eq.) and SPhos-Pd-G2 (30.0 mg, 41.7 μmol, 0.20 eq.). The mixture was degassed and stirred at 90° C. for 3 hours under nitrogen atmosphere. The mixture was diluted with water (2.0 mL), extracted with ethyl acetate (2.0 mL×3), dried with anhydrous sodium sulfate and filtered. The filtrate was concentrated under reduced pressure. The residue was purified by prep-TLC (silica gel plate, petroleum ether/ethyl acetate 1:2) to give tert-buty/2-(7-(5-(3-cyanobenzo[b]thiophen-2-yl)-1-methyl-1H-pyrazol-4-yl)-4-oxo-3,4-dihydrophthalazin-1-yl)azetidine-1-carboxylate (85 mg, 60% yield) as a yellow solid. LCMS [M+1]⁺: 539.4. ¹H NMR (400 MHz, DMSO-d₆) δ=12.63 (s, 1H), 8.31-8.28 (m, 1H), 8.27 (s, 1H), 8.19 (d, J=8.4 Hz, 1H), 8.01-7.98 (m, 1H), 7.76 (d, J=8.0 Hz, 1H), 7.70-7.66 (m, 2H), 7.65 (d, J=1.6 Hz, 1H), 5.31-5.14 (m, 1H), 3.95 (s, 3H), 3.73-3.64 (m, 2H), 2.10-2.00 (m, 1H), 1.94-1.82 (m, 1H), 1.07 (s, 9H).

Step 4: To a solution of tert-butyl 2-(7-(5-(3-cyanobenzo[b]thiophen-2-yl)-1-methyl-1H-pyrazol-4-yl)-4-oxo-3,4-dihydrophthalazin-1-yl)azetidine-1-carboxylate (50.0 mg, 92.8 μmol, 1.00 eq.) in dichloromethane (1.50 mL) was added trifluoroacetic acid (770 mg, 6.75 mmol, 0.50 mL, 73.0 eq.). The mixture was stirred at 25° C. for 1 hour before being concentrated. The residue was purified by prep-HPLC (HCl condition; column: Phenomenex luna C18 150×25 mm×10 μm; mobile phase: [water (0.05% HCl)-ACN]; B %: 11%-41%, 11 min) to give 2-(4-(4-(azetidin-2-yl)-1-oxo-1,2-dihydrophthalazin-6-yl)-1-methyl-1H-pyrazol-5-yl)benzo[b]thiophene-3-carbonitrile (15.9 mg, 34.4% yield) as a white solid. LCMS [M+1]⁺: 439.3. ¹H NMR (400 MHz, DMSO-d₆) δ=12.97 (s, 1H), 10.34-10.11 (m, 1H), 9.22 (s, 1H), 8.48-8.43 (m, 1H), 8.31-8.25 (m, 1H), 8.13 (d, J=8.4 Hz, 1H), 8.03-7.97 (m, 1H), 7.89 (s, 1H), 7.72-7.64 (m, 2H), 7.51 (dd, J=8.4, 1.2 Hz, 1H), 6.04-5.96 (m, 1H), 4.08-3.99 (m, 1H), 3.93 (s, 3H), 3.79-3.69 (m, 1H), 2.87-2.73 (m, 1H), 2.44 (s, 1H).

### Example 9-9, Example 9-10 and Example 9-12

Examples 9-9 and 9-10 were prepared following the same Steps 1-5 as for preparation of Example 9-8 starting from tert-butyl (3-formyloxetan-3-yl)carbamate (610 mg, 3.03 mmol, 1.00 eq.) to produce Example 9-9 (2.24 mg) as an off-white solid and starting from tert-butyl N-(1-formylcyclobutyl)carbamate (3.00 g, 15.06 mmol, 1.00 eq.) to produce Example 9-10 (6.00 mg) as a yellow gum. The properties and the structures are described in Table 9.

Example 9-12 was prepared following the same Steps 1-4 as for preparation of Example 9-11 starting from tert-butyl 3-(methoxy(methyl)carbamoyl)azetidine-1-carboxylate (684 mg, 2.80 mmol, 1.10 eq.) to produce Example 9-12 (11.7 mg) as a white solid. The properties and the structure are described in Table 9

TABLE 9

| Example | Structure | Compound Name and Characterization |
|---|---|---|
| 9-8 | 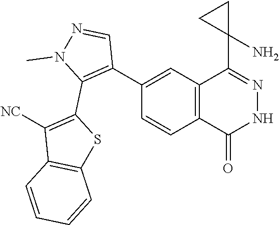 [SMILES: Cn1ncc(-c2ccc3c(=O)[nH]nc(C4(N)CC4)c3c2)c1-c1sc2ccccc2c1C#N] | 2-(4-(4-(1-aminocyclopropyl)-1-oxo-1,2- dihydrophthalazin-6-yl)-1-methyl-1H- pyrazol-5-yl)benzo[b]thiophene-3- carbonitrile. LCMS [M + 1]⁺ = 439. ¹H NMR (400 MHz, MeOD-d₄) δ = 8.34 (d, J = 8.4 Hz, 1H), 8.16 (s, 1H), 8.15-8.11 (m, 1H), 8.05-8.00 (m, 1H), 7.93 (d, J = 1.2 Hz, 1H), 7.88 (dd, J = 1.6, 8.4 Hz, 1H), 7.73-7.63 (m, 2H), 4.01 (s, 3H), 1.10-1.03 (m, 2H), 1.02-0.96 (m, 2H). |
| 9-9 | 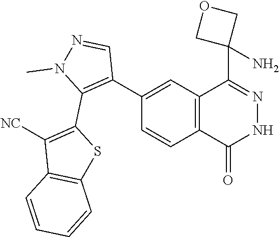 [SMILES: Cn1ncc(-c2ccc3c(=O)[nH]nc(C4(N)COC4)c3c2)c1-c1sc2ccccc2c1C#N] | 2-(4-(4-(3-aminooxetan-3-yl)-1-oxo-1,2- dihydrophthalazin-6-yl)-1-methyl-1H- pyrazol-5-yl)benzo[b]thiophene-3- carbonitrile. LCMS [M + 1]⁺: 454.9. ¹H NMR (400 MHz, CD₃OD) δ = 8.49 (d, J = 8.4 Hz, 1H), 8.29 (d, J = 1.2 Hz, 1H), 8.12 (s, 1H), 8.08-8.03 (m, 2H), 8.01- 7.96 (m, 1H), 7.70-7.60 (m, 2H), 4.72- 4.43 (m, 4H), 4.01 (s, 3H). |
| 9-10 | 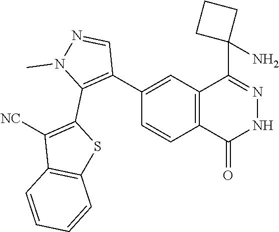 [SMILES: Cn1ncc(-c2ccc3c(=O)[nH]nc(C4(N)CCC4)c3c2)c1-c1sc2ccccc2c1C#N] | 2-(4-(4-(1-aminocyclobutyl)-1-oxo-1,2- dihydrophthalazin-6-yl)-1-methyl-1H- pyrazol-5-yl) benzo[b]thiophene-3- carbonitrile. LCMS [M + 1]⁺ = 453.1.¹H NMR (400 MHz, DMSO-d₆) δ = 12.92 (s, 1H), 8.71 (s, 3H), 8.30 (d, J = 8.4 Hz, 1H), 8.25 (s, 1H), 8.25-8.19 (m, 1H), 7.98-7.92 (m, 1H), 7.86 (d, J = 8.4 Hz, 1H), 7.69-7.60 (m, 2H), 7.47 (s, 1H), 3.99 (s, 3H), 2.44 (s, 1H), 2.40-2.36 (m, 1H), 2.10 (d, J = 4.8 Hz, 2H), 1.93-1.86 (m, 1H), 1.44-1.35 (m,l1H). |
| 9-11 | 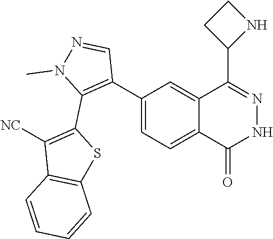 [SMILES: Cn1ncc(-c2ccc3c(=O)[nH]nc(C4CCN4)c3c2)c1-c1sc2ccccc2c1C#N] | 2-(4-(4-(azetidin-2-yl)-1-oxo-1,2- dihydrophthalazin-6-yl)-1-methyl-1H- pyrazol-5-yl)benzo[b]thiophene-3- carbonitrile. LCMS [M + 1]⁺: 439.3. ¹H NMR (400 MHz, DMSO-d₆) δ = 12.97 (s, 1H), 10.34-10.11 (m, 1H), 9.22 (s, 1H), 8.48-8.43 (m, 1H), 8.31-8.25 (m, 1H), 8.13 (d, J = 8.4 Hz, 1H), 8.03-7.97 (m, 1H), 7.89 (s, 1H), 7.72-7.64 (m, 2H), 7.51 (dd, J = 8.4, 1.2 Hz, 1H), 6.04-5.96 (m, 1H), 4.08-3.99 (m, 1H), 3.93 (s, 3H), 3.79-3.69 (m, 1H), 2.87-2.73 (m, 1H), 2.44 (s, 1H). |
| 9-12 | 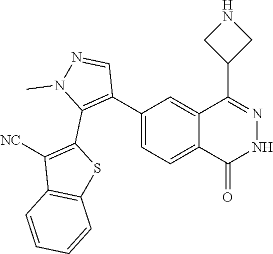 [SMILES: Cn1ncc(-c2ccc3c(=O)[nH]nc(C4CNC4)c3c2)c1-c1sc2ccccc2c1C#N] | 2-[4-[4-(azetidin-3-yl)-1-oxo-2H- phthalazin-6-yl]-2-methyl-pyrazol-3- yl]benzothiophene-3-carbonitrile. LCMS [M + 1]⁺: 439.1. ¹H NMR (400 MHz, DMSO-d₆) δ = 12.77 (s, 1H), 9.38-8.43 (m, 2H), 8.34-8.22 (m, 2H), 8.16 (d, J = 8.0 Hz, 1H), 8.03-7.93 (m, 1H), 7.74- 7.64 (m, 2H), 7.62-7.54 (m, 2H), 4.51- 4.37 (m, 1H), 4.18-4.06 (m, 4H), 3.96 (s, 3H). |

### Coupling Method (CM) and Purification Methods (PM) for the Preparation of Examples in Table 10

### General Coupling Method 10

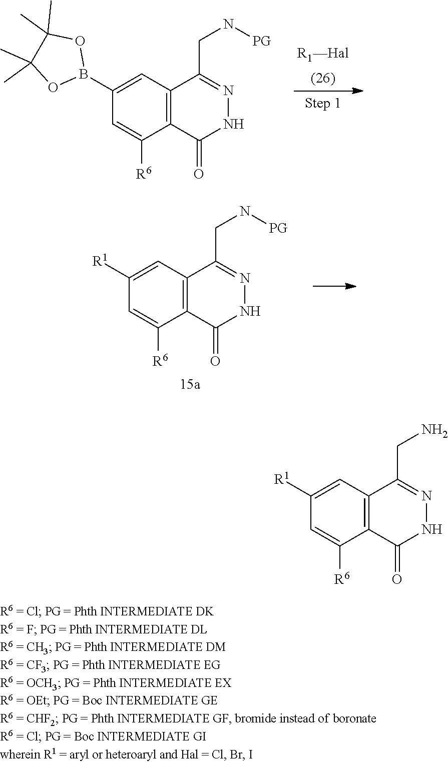

Step 1: A mixture of the appropriate aryl/heteroaryl-halide 26 (390 μmol, 1.5 eq.), intermediate AN (260 μmol, 1.00 eq.), palladium catalyst such as Pd(dppf)Cl₂, Pd(dtbpf)Cl₂ or Ad₂ n-BuP)—Pd (26.0 μmol, 0.10 eq.), sodium bicarbonate (0.521 μmol, 2.0-3.0 eq.) in dioxane (1.0 mL) and water (0.2 mL) was purged with nitrogen 3 times. The mixture was then stirred at 80° C. for 2 hours. After such time the mixture was purified using the methods described for CM 10A-1 or CM 10A-2 to give coupling product 15a.

Step 2: To a solution of corresponding coupling product 15a in ethyl alcohol (1.0 mL) was added hydrazine hydrate (20 eq.). The mixture was stirred at 80° C. for 1 hour. After such time the mixture was concentrated in vacuo and the residue purified by prep-HPLC according to one of the purification methods (PM) 4-1 through 4-25.

### CM 10A-1

### Example 10-1

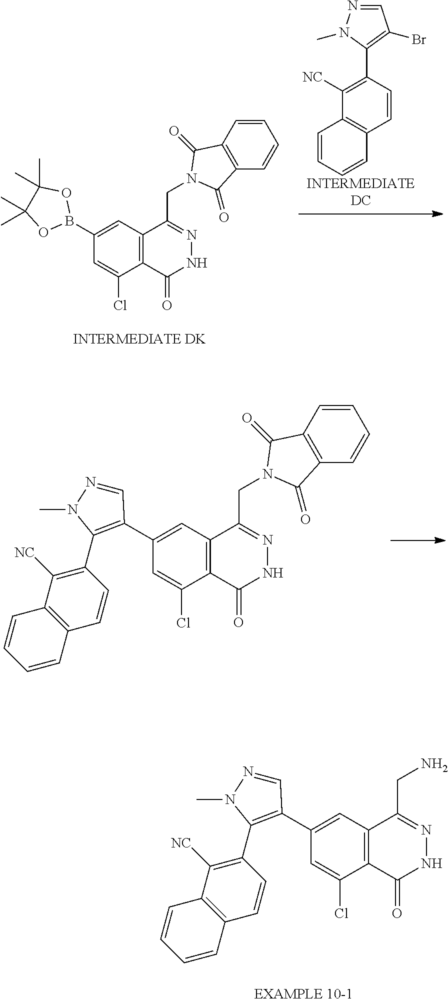

Step 1: A mixture of 2-((5-chloro-4-oxo-7-(4,4,5,5-tetramethyl-1,3,2-dioxaborolan-2-yl)-3,4-dihydrophthalazin-1-yl)methyl)isoindoline-1,3-dione, Intermediate DK (35 mg, 0.75 mmol, 1.00 eq.), 2-(4-bromo-2-methyl-pyrazol-3-yl)naphthalene-1-carbonitrile (24 mg, 0.75 mmol, 1.00 eq.), Pd(dtbpf)Cl₂ (4.9 mg, 0.08 mmol, 0.10 eq.), sodium bicarbonate (19 mg, 0.23 mmol, 3.00 eq.) in dioxane (2.0 mL) and water (0.4 mL) was degassed and purged with nitrogen for 3 times, and then the mixture was stirred at 80° C. for 2 hours. The mixture was then concentrated under reduced pressure and the residue purified by prep-TLC (SiO₂, dichloromethane/methyl alcohol 10:1) to give 2-[4-[8-chloro-4-[(1,3-dioxoisoindolin-2-yl)methyl]-1-oxo-2H-phthalazin-6-yl]-2-methyl-pyrazol-3-yl]naphthalene-1-carbonitrile (20 mg, 0.035 mmol, 47% yield) as a yellow solid. LCMS [M+1]⁺=571.2.

Step 2: A mixture of 2-[4-[8-chloro-4-[(1,3-dioxoisoindolin-2-yl)methyl]-1-oxo-2H-phthalazin-6-yl]-2-methyl-pyrazol-3-yl]naphthalene-1-carbonitrile (15 mg, 0.026 mmol, 1.00 eq.) and hydrazine hydrate (0.525 mmol, 25 μL, 20.0 eq.) in ethyl alcohol (0.8 mL) was degassed and purged with nitrogen for 3 times, and then the mixture was stirred at 80° C. for 1 hour. The mixture was then concentrated and the residue purified by prep-HPLC methods PM 4-6 to give to give 2-[4-[4-(aminomethyl)-8-chloro-1-oxo-2H-phthalazin-6-yl]-2-methyl-pyrazol-3-yl]naphthalene-1-carbonitrile, Example 10-1 (10 mg, 0.018 mmol, 69% yield) as an off-white solid.

### CM 10A-2

### Example 10-16

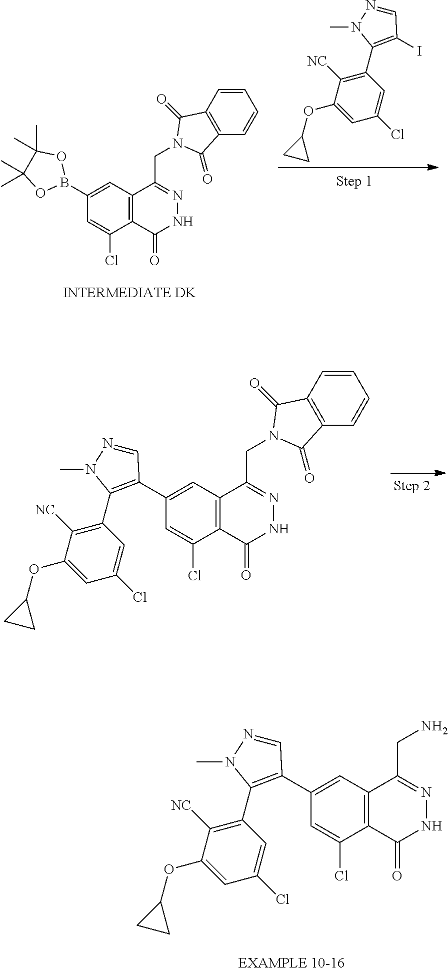 [SMILES: CC1(C)OB(c2cc(Cl)c3c(=O)[nH]nc(CN4C(=O)c5ccccc5C4=O)c3c2)OC1(C)C ; Cn1ncc(I)c1-c1cc(Cl)cc(OC2CC2)c1C#N ; Cn1ncc(-c2cc(Cl)c3c(=O)[nH]nc(CN4C(=O)c5ccccc5C4=O)c3c2)c1-c1cc(Cl)cc(OC2CC2)c1C#N ; Cn1ncc(-c2cc(Cl)c3c(=O)[nH]nc(CN)c3c2)c1-c1cc(Cl)cc(OC2CC2)c1C#N]

Step 1: A mixture of 4-chloro-2-(cyclopropoxy)-6-(4-iodo-2-methyl-pyrazol-3-yl)benzonitrile, Intermediate DK (18 mg, 0.45 mmol, 1.00 eq.), 2-[[5-chloro-4-oxo-7-(4,4,5,5-tetramethyl-1,3,2-dioxaborolan-2-yl)-3H-phthalazin-1-yl]methyl]isoindoline-1,3-dione (25 mg, 0.54 mmol, 1.20 eq.), sodium bicarbonate (11 mg, 0.13 mmol, 3.00 eq.), [2-(2-aminophenyl)phenyl]palladium(1+);bis(1-adamantyl)-butyl-phosphane;methanesulfonate (Ad₂ n-BuP)—Pd (3.3 mg, 0.005 mmol, 0.10 eq.) in water (0.2 mL) and dioxane (1.0 mL) was degassed and purged with nitrogen 3 times, and then the mixture was stirred at 80° C. for 2 hours. After such time the reaction mixture was concentrated and the residue was diluted with ethyl acetate (30 mL) and washed with brine (40 mL×3), dried over sodium sulfate, filtered, concentrated and the residue was purified by prep-TLC (SiO₂, Petroleum ether:Ethyl acetate 3:1) to give 4-chloro-2-[4-[8-chloro-4-[(1,3-dioxoisoindolin-2-yl)methyl]-1-oxo-2H-phthalazin-6-yl]-2-methyl-pyrazol-3-yl]-6-(cyclopropoxy)benzonitrile (20 mg, 73% yield) as a yellow solid. LCMS [M+H]⁺=611.1.

Step 2: To a solution of 4-chloro-2-[4-[8-chloro-4-[(1,3-dioxoisoindolin-2-yl)methyl]-1-oxo-2H-phthalazin-6-yl]-2-methyl-pyrazol-3-yl]-6-(cyclopropoxy)benzonitrile (15 mg, 0.025 mmol, 1.00 eq.) in ethyl alcohol (2 mL) was added hydrazine hydrate (2.5 mg, 0.049 mmol, 2.00 eq.). The mixture was stirred at 25° C. for 2 hours. The pH of the reaction mixture was then adjusted to pH 7 with hydrochloric acid (6.0 M, 0.5 mL), then concentrated and the residue was purified by prep-HPLC methods PM 4-6 to give 2-[4-[4-(aminomethyl)-8-chloro-1-oxo-2H-phthalazin-6-yl]-2-methyl-pyrazol-3-yl]-4-chloro-6-(cyclopropoxy)benzonitrile (3 mg, 21% yield, HCl) as a yellow gum.

### CM 10A-3

Following the same method as for CM 10A-2, with sodium bicarbonate (3 eq.) replaced by K₃PO₄ (3 eq.) and using coupling intermediates GF and GG

### CM 10A-4

Following the same method as for CM 10A-1, with solvent dioxane/water (5:1) replaced by DMF.

### CM 10A-5

Step 1: Following the same method as for CM 10A-1, with sodium bicarbonate (3 eq.) replaced by potassium carbonate (2.5 eq.).

Step 2: To a solution of tert-butyl ((5-R⁶-4-oxo-7-R1-3,4-dihydrophthalazin-1-yl)methyl)carbamate Suzuki coupling product 15a from step 1 (180 μmol, 1.00 eq) in dioxane (1.0 mL) was added HCl in dioxane (4M, 0.82 mL, 18.3 eq.). The mixture was stirred at 25° C. for 2 hours. The reaction was concentrated in vacuo and the residue purified by prep-HPLC according to purification methods PM 4-1 through PM 4-19

### CM 10A-6

Step 1: Following the same method as for CM 10A-2, with sodium bicarbonate (3 eq.) replaced by potassium carbonate (3 eq.) and Intermediate DK replaced by Intermediate GI.

Step 2: A mixture of tert-butyl ((5-R⁶-4-oxo-7-R1-3,4-dihydrophthalazin-1-yl)methyl)carbamate Suzuki coupling product 15a from step 1 (18 μmol, 1.00 eq) in hydrochloric acid/ethyl acetate (4.00 M, 0.50 mL, 109 eq.) was stirred at 25° C. for 2 h. The reaction was concentrated in vacuo and the residue purified by prep-HPLC according to purification method PM 4-1 through PM 4-19

### CM 10A-7

Following the same method as for CM 10A-2, with sodium bicarbonate (3 eq.) replaced by K₃PO₄ (3 eq.) and Dioxane/Water solvent was replaced by DMAc.

Following the teachings of the General Reaction Schemes, the general coupling method CM 10A-1, CM 10A-2, CM 10A-3, CM 10A-4, CM 10A-5, CM 10A-6 and CM 10-A7 and using purification methods 4-4 to 4-19, the Examples 10-1 to 10-26 were prepared as shown in Table 10.

TABLE 10

| Example | Structure | CM | PM | Compound Name and Characterization |
|---|---|---|---|---|
| 10-1 | 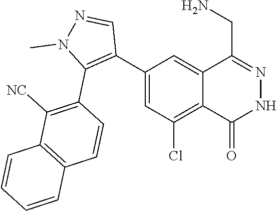 [SMILES: Cn1ncc(-c2cc(Cl)c3c(=O)[nH]nc(CN)c3c2)c1-c1ccc2ccccc2c1C#N] | 10A-1 | 4-4 | 2-(4-(4-(aminomethyl)-8-chloro-1-oxo-1,2- dihydrophthalazin-6-yl)-1-methyl-1H-pyrazol- 5-yl)-1-naphthonitrile LCMS [M + 1]⁺ = 441.1; ¹H NMR (400 MHz, DMSO-d₆) δ = 12.81 (s, 1H), 8.57 (br d, J = 8.4 Hz, 1H), 8.42 (s, 1H), 8.40-8.24 (m, 4H), 8.16 (br d, J = 8.0 Hz, 1H), 7.95-7.81 (m, 3H), 7.72 (s, 1H), 7.30 (s, 1H), 4.33-4.02 (m, 2H), 3.78 (s, 3H) |
| 10-2 | 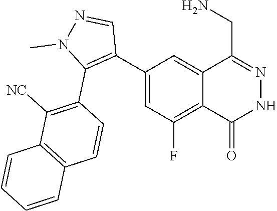 [SMILES: Cn1ncc(-c2cc(F)c3c(=O)[nH]nc(CN)c3c2)c1-c1ccc2ccccc2c1C#N] | 10A-1 | 4-4 | 2-(4-(4-(aminomethyl)-8-fluoro-1-oxo-1,2- dihydrophthalazin-6-yl)-1-methyl-1H-pyrazol- 5-yl)-1-naphthonitrile LCMS [M + 1]⁺ = 425.1; ¹H NMR (400 MHz, DMSO-d₆) δ = 12.81 (s, 1H), 8.57 (d, 1H), 8.39 (s, 1H), 8.30-8.25 (m, 4H), 8.15 (d, J = 8.0 Hz, 1H), 7.93-7.80 (m, 3H), 7.60 (s, 1H), 7.05 (d, 1H), 4.27-4.08 (m, 2H), 3.78 (s, 3H) |
| 10-3 | 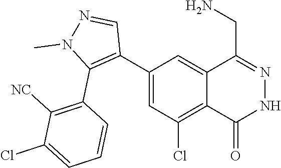 [SMILES: Cn1ncc(-c2cc(Cl)c3c(=O)[nH]nc(CN)c3c2)c1-c1cccc(Cl)c1C#N] | 10A-1 | 4-5 | 2-(4-(4-(aminomethyl)-8-chloro-1-oxo-1,2- dihydrophthalazin-6-yl)-1-methyl-1H-pyrazol- 5-yl)-6-chlorobenzonitrile LCMS [M + 1]⁺ = 425.2; ¹H NMR (400 MHz, DMSO-d₆) δ = 12.86 (s, 1H), 8.39-8.31 (m, 4H), 8.04-7.96 (m, 2H), 7.84 (dd, J = 1.2, 7.2 Hz, 1H), 7.59 (d, J = 1.6 Hz, 1H), 7.42 (d, J = 1.6 Hz, 1H), 4.27-4.12 (m, 2H), 3.77 (s, 3H) |
| 10-4 | 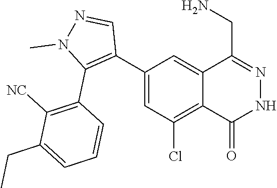 [SMILES: CCc1cccc(-c2c(-c3cc(Cl)c4c(=O)[nH]nc(CN)c4c3)cnn2C)c1C#N] | 10A-1 | 4-5 | 2-(4-(4-(aminomethyl)-8-chloro-1-oxo-1,2- dihydrophthalazin-6-yl)-1-methyl-1H-pyrazol- 5-yl)-6-ethylbenzonitrile LCMS [M + 1]⁺ = 419.2; ¹H NMR (400 MHz, DMSO-d₆) δ = 12.82 (s, 1H), 8.48 (br s, 3H), 8.42 (s, 1H), 7.91 (t, J = 8.0 Hz, 1H), 7.77 (d, J = 7.6 Hz, 1H), 7.65 (d, J = 7.6 Hz, 1H), 7.58 (d, J = 1.6 Hz, 1H), 7.34 (s, 1H), 4.23-4.06 (m, 2H), 3.73 (s, 3H), 2.87 (q, J = 7.6 Hz, 2H), 1.24 (t, J = 7.6 Hz, 3H) |
| 10-5 | 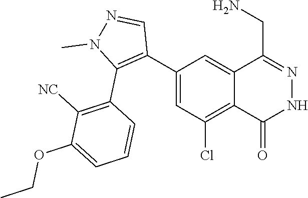 [SMILES: CCOc1cccc(-c2c(-c3cc(Cl)c4c(=O)[nH]nc(CN)c4c3)cnn2C)c1C#N] | 10A-1 | 4-5 | 2-(4-(4-(aminomethyl)-8-chloro-1-oxo-1,2- dihydrophthalazin-6-yl)-1-methyl-1H-pyrazol- 5-yl)-6-ethoxybenzonitrile LCMS [M + 1]⁺ = 435.2; ¹H NMR (400 MHz, DMSO-d₆) δ = 12.84 (s, 1H), 8.44-8.26 (m, 4H), 7.89 (t, J = 8.0 Hz, 1H), 7.61 (br d, J = 4.8 Hz, 1H), 7.50 (d, J = 8.8 Hz, 1H), 7.40 (br d, J = 2.8 Hz, 1H), 7.32 (d, J = 7.6 Hz, 1H), 4.37-4.27 (m, 2H), 4.25-4.06 (m, 2H), 3.72 (s, 3H), 1.38 (t, J = 7.2 Hz, 3H) |
| 10-6 | 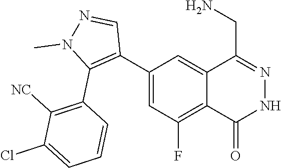 [SMILES: Cn1ncc(-c2cc(F)c3c(=O)[nH]nc(CN)c3c2)c1-c1cccc(Cl)c1C#N] | 10A-1 | 4-5 | 2-(4-(4-(aminomethyl)-8-fluoro-1-oxo-1,2- dihydrophthalazin-6-yl)-1-methyl-1H-pyrazol- 5-yl)-6-chlorobenzonitrile LCMS [M + 1]⁺ = 409.2; ¹H NMR (400 MHz, DMSO-d₆) δ = 12.86 (s, 1H), 8.39-8.28 (m, 4H), 8.04-7.93 (m, 2H), 7.81 (dd, J = 1.2, 7.2 Hz, 1H), 7.44 (s, 1H), 7.24 (d, J = 12.4 Hz, 1H), 4.25-4.12 (m, 2H), 3.76 (s, 3H) |
| 10-7 | 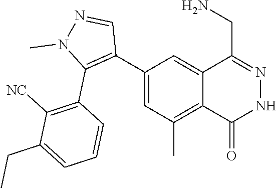 [SMILES: CCc1cccc(-c2c(-c3cc(C)c4c(=O)[nH]nc(CN)c4c3)cnn2C)c1C#N] | 10A-1 | 4-4 | 2-(4-(4-(aminomethyl)-8-methyl-1-oxo-1,2- dihydrophthalazin-6-yl)-1-methyl-1H-pyrazol- 5-yl)-6-ethylbenzonitrile LCMS [M + 1]⁺ = 399.2; ¹H NMR (400 MHz, DMSO-d₆) δ = 12.64 (s, 1H), 8.31 (s, 4H), 7.92-7.85 (m, 1H), 7.75 (d, J = 7.2 Hz, 1H), 7.62 (dd, J = 0.8, 7.6 Hz, 1H), 7.39 (s, 1H), 7.23 (s, 1H), 4.18-4.01 (m, 2H), 3.74 (s, 3H), 2.87 (q, J = 7.6 Hz, 2H), 2.64 (s, 3H), 1.24 (L, J = 7.6 Hz, 3H) |
| 10-8 | 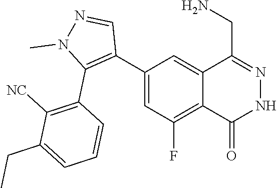 [SMILES: CCc1cccc(-c2c(-c3cc(F)c4c(=O)[nH]nc(CN)c4c3)cnn2C)c1C#N] | 10A-1 | 4-4 | 2-(4-(4-(aminomethyl)-8-fluoro-1-oxo-1,2- dihydrophthalazin-6-yl)-1-methyl-1H-pyrazol- 5-yl)-6-ethylbenzonitrile LCMS [M + 1]⁺ = 403.2; ¹H NMR (400 MHz, DMSO-d₆) δ = 12.82 (s, 1H), 8.48 (br s, 3H), 8.39 (s, 1H), 7.90 (t, J = 7.6 Hz, 1H), 7.76 (d, J = 7.6 Hz, 1H), 7.64 (d, J = 7.6 Hz, 1H), 7.40 (d, J = 0.8 Hz, 1H), 7.18 (d, J = 12.4 Hz, 1H), 4.20-3.99 (m, 2H), 3.73 (s, 3H), 2.88 (q, J = 7.6 Hz, 2H), 1.24 (t, J = 7.6 Hz, 3H) |
| 10-9 | 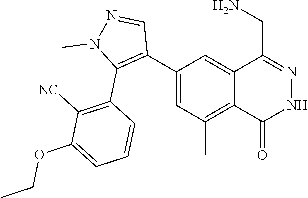 [SMILES: CCOc1cccc(-c2c(-c3cc(C)c4c(=O)[nH]nc(CN)c4c3)cnn2C)c1C#N] | 10A-1 | 4-4 | 2-(4-(4-(aminomethyl)-8-methyl-1-oxo-1,2- dihydrophthalazin-6-yl)-1-methyl-1H-pyrazol- 5-yl)-6-ethoxybenzonitrile LCMS [M + 1]⁺ = 415.2; ¹H NMR (400 MHz, DMSO-d₆) δ = 12.64 (s, 1H), 8.37-8.26 (m, 3H), 7.87 (dd, J = 7.6, 8.8 Hz, 1H), 7.48 (d, J = 8.8 Hz, 1H), 7.41 (s, 1H), 7.35-7.26 (m, 2H), 4.30 (dq, J = 2.8, 6.8 Hz, 2H), 4.21-4.01 (m, 2H), 3.72 (s, 3H), 2.67 (s, 3H), 1.39 (t, J = 7.2 Hz, 3H) |
| 10-10 | 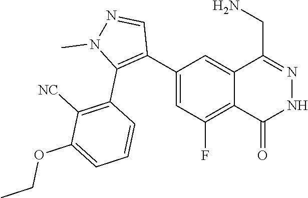 [SMILES: CCOc1cccc(-c2c(-c3cc(F)c4c(=O)[nH]nc(CN)c4c3)cnn2C)c1C#N] | 10A-1 | 4-4 | 2-(4-(4-(aminomethyl)-8-fluoro-1-oxo-1,2- dihydrophthalazin-6-yl)-1-methyl-1H-pyrazol- 5-yl)-6-ethoxybenzonitrile LCMS [M + 1]⁺ = 419.2; ¹H NMR (400 MHz, DMSO-d₆) δ = 12.84 (s, 1H), 8.45-8.25 (m, 4H), 7.89 (dd, J = 7.6, 8.4 Hz, 1H), 7.49 (d, J = 8.0 Hz, 1H), 7.31 (d, J = 7.6 Hz, 1H), 7.17 (d, J = 12.4 Hz, 1H), 4.35-4.26 (m, 2H), 4.25-4.07 (m, 2H), 3.72 (s, 3H), 1.39 (t, J = 6.8 Hz, 3H) |
| 10-11 | 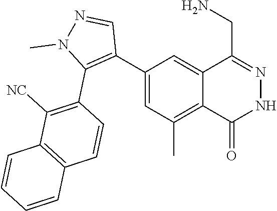 [SMILES: Cc1cc(-c2cnn(C)c2-c2ccc3ccccc3c2C#N)cc2c(CN)n[nH]c(=O)c12] | 10A-1 | 4-6 | 2-(4-(4-(aminomethyl)-8-methyl-1-oxo-1,2- dihydrophthalazin-6-yl)-1-methyl-1H-pyrazol- 5-yl)-1-naphthonitrile LCMS [M + 1]⁺ = 421.1; ¹H NMR (400 MHz, DMSO-d₆) δ = 12.60 (s, 1H), 8.56 (d, J = 8.4 Hz, 1H), 8.42 (br s, 3H), 8.34 (s, 1H), 8.29 (d, J = 7.6 Hz, 1H), 8.15 (d, J = 8.4 Hz, 1H), 7.93-7.82 (m, 3H), 7.54 (s, 1H), 7.19 (s, 1H), 4.25-4.12 (m, 1H), 4.07-3.94 (m, 1H), 3.77 (s, 3H), 2.52 (s, 3H) |
| 10-12 | 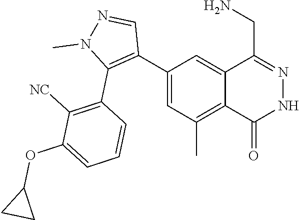 [SMILES: Cc1cc(-c2cnn(C)c2-c2cccc(OC3CC3)c2C#N)cc2c(CN)n[nH]c(=O)c12] | 10A-1 | 4-6 | 2-(4-(4-(aminomethyl)-8-methyl-1-oxo-1,2- dihydrophthalazin-6-yl)-1-methyl-1H-pyrazol- 5-yl)-6-cyclopropoxybenzonitrile LCMS [M + 1]⁺ = 427.2; ¹H NMR (400 MHz, DMSO- d₆) δ = 12.63 (s, 1H), 8.44 (br s, 3H), 8.28 (s, 1H), 7.93 (dd, J = 7.6, 8.8 Hz, 1H), 7.75 (dd, J = 0.8, 8.8 Hz, 1H), 7.37 (d, J = 1.2 Hz, 1H), 7.35 (dd, J = 0.8, 7.6 Hz, 1H), 7.31 (s, 1H), 4.22-4.09 (m, 2H), 4.08-3.95 (m, 1H), 3.72 (s, 3H), 2.67 (s, 3H), 0.94-0.86 (m, 2H), 0.83-0.74 (m, 2H) |
| 10-13 | 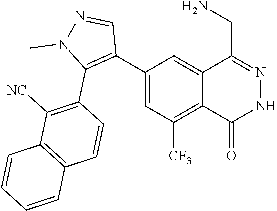 [SMILES: Cn1ncc(-c2cc(C(F)(F)F)c3c(=O)[nH]nc(CN)c3c2)c1-c1ccc2ccccc2c1C#N] | 10A-1 | 4-6 | 2-(4-(4-(aminomethyl)-1-oxo-8-(trifluoro- methyl)-1,2-dihydrophthalazin-6-yl)-1-methyl- 1H-pyrazol-5-yl)-1-naphthonitrile LCMS [M + 1]⁺ = 475.2; ¹H NMR (400 MHz, DMSO- d₆) δ = 12.98 (s, 1H), 8.58 (d, J = 8.4 Hz, 1H), 8.52 (s, 1H), 8.39 (br d, J = 1.2 Hz, 3H), 8.30 (d, J = 7.6 Hz, 1H), 8.19 (s, 1H), 8.14 (d, J = 8.0 Hz, 1H), 7.93-7.84 (m, 3H), 7.53 (s, 1H), 4.48-4.24 (m, 2H), 3.80 (s, 3H) |
| 10-14 | 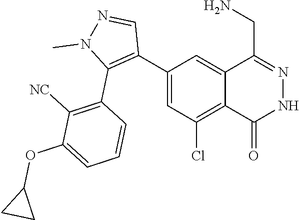 [SMILES: Cn1ncc(-c2cc(Cl)c3c(=O)[nH]nc(CN)c3c2)c1-c1cccc(OC2CC2)c1C#N] | 10A-1 | 4-6 | 2-(4-(4-(aminomethyl)-8-chloro-1-oxo-1,2- dihydrophthalazin-6-yl)-1-methyl-1H-pyrazol- 5-yl)-6-cyclopropoxybenzonitrile LCMS [M + 1]⁺ = 447.2; ¹H NMR (400 MHz, DMSO- d₆) δ = 12.83 (s, 1H), 8.47 (br s, 3H), 8.38 (s, 1H), 7.96 (dd, J = 7.7, 8.6 Hz, 1H), 7.81-7.75 (m, 1H), 7.57 (d, J = 1.6 Hz, 1H), 7.42 (d, J = 1.6 Hz, 1H), 7.39 (dd, J = 0.6, 7.6 Hz, 1H), 4.26-4.16 (m, 2H), 4.15-4.06 (m, 1H), 3.73 (s, 3H), 0.94-0.76 (m, 4H) |
| 10-15 | 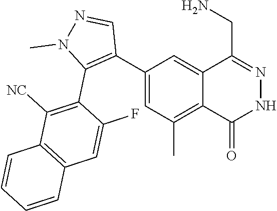 [SMILES: Cc1cc(-c2cnn(C)c2-c2c(F)cc3ccccc3c2C#N)cc2c(CN)n[nH]c(=O)c12] | 10A-1 | 4-6 | 2-(4-(4-(aminomethyl)-8-methyl-1-oxo-1,2- dihydrophthalazin-6-yl)-1-methyl-1H-pyrazol- 5-yl)-3-fluoro-1-naphthonitrile LCMS [M + 1]⁺ = 439.2; ¹H NMR (400 MHz, DMSO- d6) δ = 12.15 (s, 1H), 8.53 (d, J = 10.0 Hz, 1H), 8.31 (s, 1H), 8.27-8.23 (m, 1H), 8.19-8.13 (m, 1H), 7.92-7.83 (m, 2H), 7.53 (d, J = 0.8 Hz, 1H), 7.38 (d, J = 1.6 Hz, 1H), 3.81 (s, 3H), 3.35 (br s, 2H), 2.69 (s, 3H) |
| 10-16 | 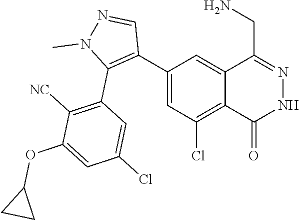 [SMILES: Cn1ncc(-c2cc(Cl)c3c(=O)[nH]nc(CN)c3c2)c1-c1cc(Cl)cc(OC2CC2)c1C#N] | 10A-2 | 4-6 | 2-(4-(4-(aminomethyl)-8-chloro-1-oxo-1,2- dihydrophthalazin-6-yl)-1-methyl-1H-pyrazol- 5-yl)-4-chloro-6-cyclopropoxybenzonitrile LCMS [M + H]⁺ = 481.1; ¹H NMR (400 MHz, DMSO-d6) δ = 12.86 (s, 1H), 8.45 (br s, 3H), 8.36 (s, 1H), 7.85 (d, J = 1.6 Hz, 1H), 7.61 (d, J = 1.2 Hz, 2H), 7.43 (d, J = 1.2 Hz, 1H), 4.30- 4.23 (m, 2H), 4.19 (br d, J = 4.4 Hz, 1H), 3.76 (s, 3H), 0.96-0.88 (m, 2H), 0.75-0.85 (m, 2H) |
| 10-17 | 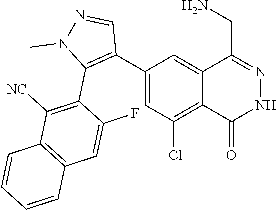 [SMILES: Cn1ncc(-c2cc(Cl)c3c(=O)[nH]nc(CN)c3c2)c1-c1c(F)cc2ccccc2c1C#N] | 10A-2 | 4-6 | 2-(4-(4-(aminomethyl)-8-chloro-1-oxo-1,2- dihydrophthalazin-6-yl)-1-methyl-1H-pyrazol- 5-yl)-3-fluoro-1-naphthonitrile LCMS [M + 1]⁺ = 459.1; ¹H NMR (400 MHz, DMSO- d6) δ = 12.82 (s, 1H), 8.58 (d, J = 10.0 Hz, 1H), 8.52 (s, 1H), 8.49 (br s, 3H), 8.31-8.26 (m, 1H), 8.20-8.14 (m, 1H), 7.92-7.85 (m, 2H), 7.76 (d, J = 1.6 Hz, 1H), 7.30 (d, J = 1.6 Hz, 1H), 4.35-4.06 (m, 2H), 3.82 (s, 3H) |
| 10-18 | 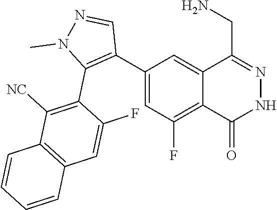 [SMILES: Cn1ncc(-c2cc(F)c3c(=O)[nH]nc(CN)c3c2)c1-c1c(F)cc2ccccc2c1C#N] | 10A-2 | 4-6 | 2-(4-(4-(aminomethyl)-8-fluoro-1-oxo-1,2- dihydrophthalazin-6-yl)-1-methyl-1H-pyrazol- 5-yl)-3-fluoro-1-naphthonitrile LCMS [M + 1]⁺ = 443.1 |
| 10-19 | 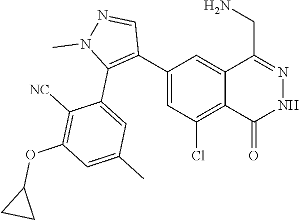 [SMILES: Cc1cc(OC2CC2)c(C#N)c(-c2c(-c3cc(Cl)c4c(=O)[nH]nc(CN)c4c3)cnn2C)c1] | 10A-2 | 4-6 | 2-(4-(4-(aminomethyl)-8-chloro-1-oxo-1,2- dihydrophthalazin-6-yl)-1-methyl-1H-pyrazol- 5-yl)-6-cyclopropoxy-4-methylbenzonitrile LCMS [M + 1]⁺ = 461.1; ¹H NMR (400 MHz, DMSO-d₆) δ = 12.83 (s, 1H), 8.50 (br s, 3H), 8.38 (s, 1H), 7.61 (s, 1H), 7.53 (d, J = 1.6 Hz, 1H), 7.49 (s, 1H), 7.23 (s, 1H), 4.23-4.12 (m, 2H), 4.11-4.01 (m, 1H), 3.72 (s, 3H), 2.53 (s, 3H), 0.94-0.86 (m, 2H), 0.80-0.74 (m, 2H) |
| 10-20 | 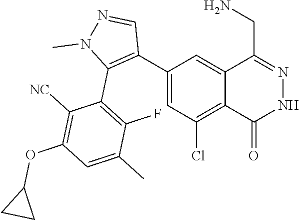 [SMILES: Cc1cc(OC2CC2)c(C#N)c(-c2c(-c3cc(Cl)c4c(=O)[nH]nc(CN)c4c3)cnn2C)c1F] | 10A-1 | 4-6 | 2-(4-(4-(aminomethyl)-8-chloro-1-oxo-1,2- dihydrophthalazin-6-yl)-1-methyl-1H-pyrazol- 5-yl)-6-cyclopropoxy-3-fluoro-4-methylbenzo- nitrile LCMS [M + 1]⁺ = 479.1; ¹H NMR (400 MHz, DMSO-d₆) δ = 12.85 (s, 1H), 8.52 (br s, 3H), 8.46 (s, 1H), 7.80 (d, J = 6.0 Hz, 1H), 7.54 (d, J = 1.6 Hz, 1H), 7.50 (d, J = 1.6 Hz, 1H), 4.27-4.05 (m, 3H), 3.76 (s, 3H), 2.48 (d, J = 1.6 Hz, 3H), 0.94-0.85 (m, 2H), 0.83-0.68 (m, 2H) |
| 10-21 | 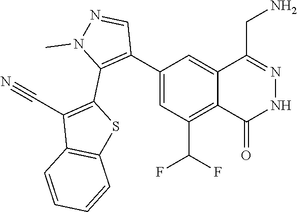 [SMILES: Cn1ncc(-c2cc(C(F)F)c3c(=O)[nH]nc(CN)c3c2)c1-c1sc2ccccc2c1C#N] | 10A-3 | 4-13 | 2-[4-[4-(aminomethyl)-8-(difluoromethyl)-1- oxo-2H-phthalazin-6-yl]-2-methyl-pyrazol-3- yl]benzothiophene-3-carbonitrile. LCMS [M + 1]⁺ = 463.0. ¹H NMR (400 MHz, DMSO- d₆) δ = 13.07 (s, 1H), 8.50 (s, 4H), 8.29-8.35 (m, 1H), 8.22 (s, 1 H), 8.07-7.99 (m, 1H), 7.99- 7.77 (m, 1H), 7.74-7.64 (m, 3H), 4.45 (d, J = 4.8 Hz, 2H), 3.94 (s, 3H). |
| 10-22 | 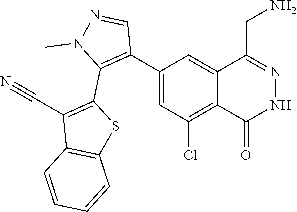 [SMILES: Cn1ncc(-c2cc(Cl)c3c(=O)[nH]nc(CN)c3c2)c1-c1sc2ccccc2c1C#N] | 10A-4 | 4-18 | 2-(4-(4-(aminomethyl)-8-chloro-1-oxo-1,2- dihydrophthalazin-6-yl)-1-methyl-1H-pyrazol- 5-yl)benzo[b]thiophene-3-carbonitrile. LCMS [M + 1]⁺: 447.1 ¹H NMR (400 MHz, DMSO- d₆) δ = 12.72-12.18 (m, 1H), 8.37 (s, 1H), 8.34- 8.28 (m, 1H), 8.25 (s, 1H), 8.06-7.96 (m, 1H), 7.75 (d, J = 1.6 Hz, 1H), 7.72-7.65 (m, 3H), 3.93 (s, 3H), 3.68 (s, 2H). |
| 10-23 | 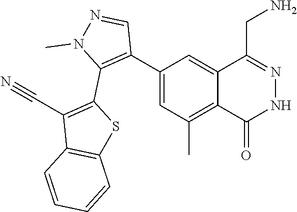 [SMILES: Cc1cc(-c2cnn(C)c2-c2sc3ccccc3c2C#N)cc2c(CN)n[nH]c(=O)c12] | 10A-1 | 4-6 | 2-[4-[4-(aminomethyl)-8-methyl-1-oxo-2H- phthalazin-6-yl]-2-methyl-pyrazol-3-yl]benzo- thiophene-3-carbonitrile. LC-MS [M + 1]⁺ = 427.2. ¹H NMR (400 MHz, CD₃OD) δ = 8.17- 8.10 (m, 2H), 8.01 (dd, J = 2.4, 6.8 Hz, 1H), 7.71-7.63 (m, 2H), 7.55 (s, 1H), 7.45 (s, 1H), 4.24 (s, 2H), 3.98 (s, 3H), 2.72 (s, 3H) |
| 10-24 | 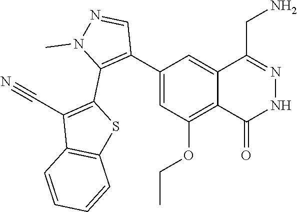 [SMILES: CCOc1cc(-c2cnn(C)c2-c2sc3ccccc3c2C#N)cc2c(CN)n[nH]c(=O)c12] | 10A-5 | 4-6 | 2-(4-(4-(aminomethyl)-8-ethoxy-1-oxo-1,2- dihydrophthalazin-6-yl)-1-methyl-1H-pyrazol- 5-yl)benzo[b]thiophene-3-carbonitrile. LCMS [M + 1]⁺ = 457.2 1H NMR (400 MHz, CD3OD) δ = 8.22 (s, 1H), 8.19-8.13 (m, 1H), 8.05-7.97 (m, 1H), 7.73-7.62 (m, 2H), 7.39 (d, J = 1.2 Hz, 1H), 6.96 (d, J = 1.2 Hz, 1H), 4.36 (s, 2H), 3.98 (s, 3H), 3.71 (q, J = 7.2 Hz, 2H), 1.05 (t, J = 7.2 Hz, 3H). |
| 10-25 | 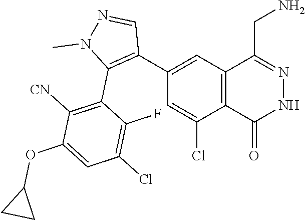 | 10A-6 | 4-19 | 2-[4-[4-(aminomethyl)-8-chloro-1-oxo-2H- phthalazin-6-yl]-2-methyl-pyrazol-3-yl]-4- chloro-6-(cyclopropoxy)-3-fluoro-benzonitrile. LCMS [ESI, M + 1]: 499.1. ¹H NMR (400 MHz, MeOD) δ = 8.53 (br d, J = 3.2 Hz, 1H), 8.19 (s, 1H), 7.95 (d, J = 6.0 Hz, 1H), 7.68 (d, J = 1.2 Hz, 1H), 7.46 (d, J = 1.2 Hz, 1H), 4.36- 4.19 (m, 2H), 4.11 (tt, J = 2.4, 5.9 Hz, 1H), 3.84 (s, 3H), 1.01-0.80 (m, 4H); |
| 10-26 | 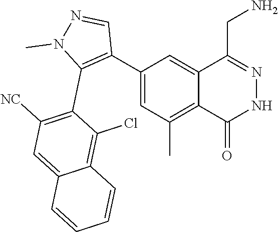 [SMILES: Cc1cc(-c2cnn(C)c2-c2c(C#N)cc3ccccc3c2Cl)cc2c(CN)n[nH]c(=O)c12] | 10A-7 | 4-6 | 3-[4-[4-(aminomethyl)-8-methyl-1-oxo-2H- phthalazin-6-yl]-2-methyl-pyrazol-3-yl]-4- chloro-naphthalene-2-carbonitrile (3.25 mg, 6.55 μmol, 7.66% yield, 99.0% purity, HCl) as a yellow solid. LCMS [M + 1]⁺ = 455.1. ¹H NMR (400 MHz, DMSO-d6) δ ppm 12.62 (s, 1 H) 8.94 (s, 1 H) 8.40-8.45 (m, 2 H) 8.23- 8.36 (m, 4 H) 8.02-8.07 (m, 1 H) 7.94-8.00 (m, 1 H) 7.59 (s, 1 H) 7.08 (s, 1 H) 4.17 (br d, J = 5.6 Hz, 2 H) 3.72 (s, 3 H) 2.52 (d, J = 2.0 Hz, 3 H) |

### Example 11-1

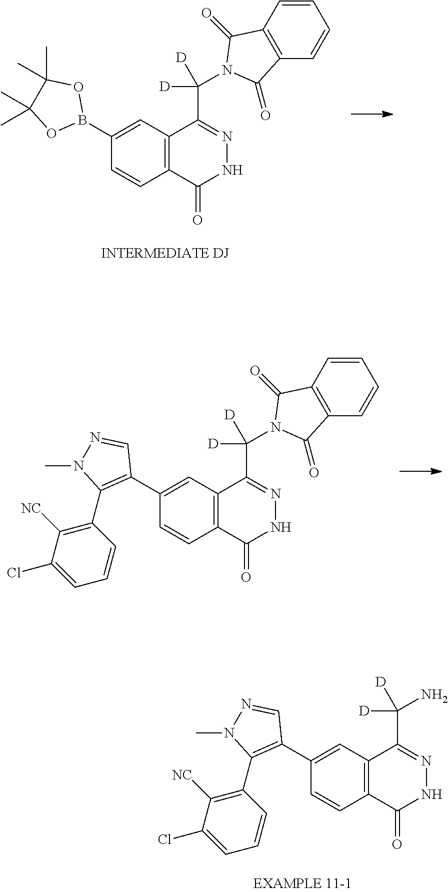 [SMILES: [2H]C([2H])(c1n[nH]c(=O)c2ccc(B3OC(C)(C)C(C)(C)O3)cc12)N1C(=O)c2ccccc2C1=O ; [2H]C([2H])(c1n[nH]c(=O)c2ccc(-c3cnn(C)c3-c3cccc(Cl)c3C#N)cc12)N1C(=O)c2ccccc2C1=O ; [2H]C([2H])(N)c1n[nH]c(=O)c2ccc(-c3cnn(C)c3-c3cccc(Cl)c3C#N)cc12]

Step 1: A mixture of 2-((4-oxo-7-(4,4,5,5-tetramethyl-1,3,2-dioxaborolan-2-yl)-3,4-dihydrophthalazin-1-yl)methyl-d2)isoindoline-1,3-dione, Intermediate DJ (100 mg, 0.152 mmol, 1.00 eq.), 2-(4-bromo-2-methyl-pyrazol-3-yl)-6-chloro-benzonitrile (58 mg, 0.197 mmol, 1.30 eq.), Pd(dtbpf)Cl₂ (10 mg, 0.015 mmol, 0.10 eq) and sodium bicarbonate (38 mg, 0.455 mmol, 3.00 eq.) in dioxane (3.0 mL) and water (0.6 mL) was degassed and purged with nitrogen 3 times and stirred at 80° C. for 2 hours. After such time the mixture was concentrated and the residue was purified by column chromatography (SiO₂, petroleum ether/ethyl acetate 10/1 to 0/1) to give 2-chloro-6-(4-(4-((1,3-dioxoisoindolin-2-yl)methyl-d2)-1-oxo-1,2-dihydrophthalazin-6-yl)-1-methyl-1H-pyrazol-5-yl)benzonitrile (50 mg, 0.089 mmol, 59% yield) as a yellow solid. LCMS [M+1]⁺=523.1.

Step 2: A mixture of 2-chloro-6-(4-(4-((1,3-dioxoisoindolin-2-yl)methyl-d2)-1-oxo-1,2-dihydrophthalazin-6-yl)-1-methyl-1H-pyrazol-5-yl)benzonitrile (50 mg, 0.089 mmol, 1.00 eq.) and hydrazine hydrate (0.044 mL, 0.89 mmol, 10.0 eq.) in ethyl alcohol (2 mL) was stirred at 80° C. for 1 hour under nitrogen atmosphere. After such time the mixture was concentrated under reduced pressure and the residue purified by prep-HPLC (Phenomenex luna C18 150×25 mm 10 μm; mobile phase: [water (0.1% trifluoroacetic acid)-ACN]; B %: 7%-37%, 10 min). to give 2-(4-(4-(aminomethyl-d2)-1-oxo-1,2-dihydrophthalazin-6-yl)-1-methyl-1H-pyrazol-5-yl)-6-chlorobenzonitrile (31 mg, 0.059 mmol, 66% yield) as a white solid. LCMS [M+1]⁺=393.1; ¹H NMR (400 MHz, DMSO-d₆) δ=12.88 (s, 1H), 8.34 (br s, 3H), 8.28 (s, 1H), 8.12 (d, J=8.4 Hz, 1H), 8.00-7.97 (dd, J=1.6, 8.4 Hz, 1H), 7.97-7.92 (t, J=7.2 Hz, 1H), 7.79-7.76 (m, 2H), 7.43 (dd, J=1.6, 8.4 Hz, 1H), 3.76 (s, 3H).

### Example 11-2

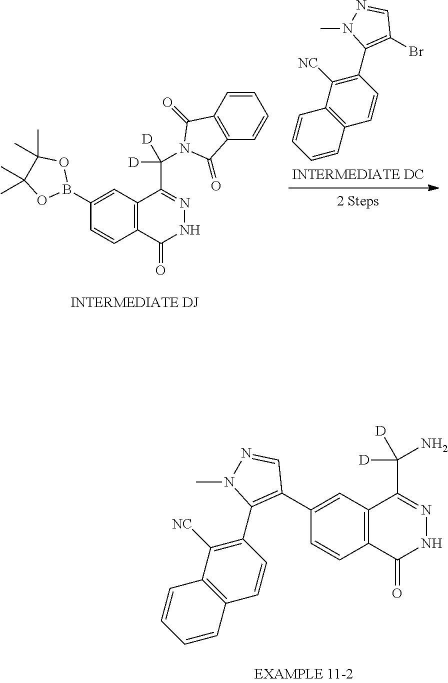

2-(4-(4-(aminomethyl-d2)-1-oxo-1,2-dihydrophthalazin-6-yl)-1-methyl-1H-pyrazol-5-yl)-1-naphthonitrile, Example 11-2 was prepared as a white solid (19 mg, 0.046 mmol, 35% yield) following the same procedure as example 11-1 using intermediate DJ (60 mg, 0.138 mol, 1.00 eq.) and Intermediate DC 2-(4-bromo-2-methyl-pyrazol-3-yl)naphthalene-1-carbonitrile (56 mg, 0.18 mmol, 1.30 eq.). LCMS [M+1]⁺=409.3; ¹H NMR (400 MHz, DMSO-d₆) δ=12.85 (s, 1H), 8.53 (d, J=8.4 Hz, 1H), 8.33-8.27 (m, 4H), 8.14 (d, J=8.0 Hz, 1H), 8.00 (d, J=8.0 Hz, 1H), 7.92-7.82 (m, 4H), 7.32 (dd, J=1.6, 8.0 Hz, 1H), 3.78 (s, 3H).

### Example 12-1

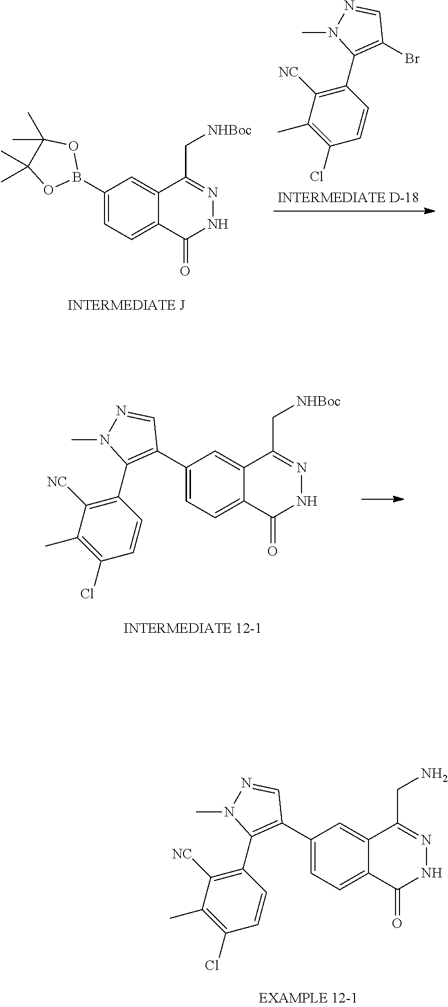 [SMILES: CC(C)(C)OC(=O)NCc1n[nH]c(=O)c2ccc(B3OC(C)(C)C(C)(C)O3)cc12 ; Cc1c(Cl)ccc(-c2c(Br)cnn2C)c1C#N ; Cc1c(Cl)ccc(-c2c(-c3ccc4c(=O)[nH]nc(CNC(=O)OC(C)(C)C)c4c3)cnn2C)c1C#N ; Cc1c(Cl)ccc(-c2c(-c3ccc4c(=O)[nH]nc(CN)c4c3)cnn2C)c1C#N]

Step 1: A mixture of 6-(4-bromo-2-methyl-pyrazol-3-yl)-3-chloro-2-methyl-benzonitrile, Intermediate D-18 (200 mg, 0.64 mmol, 1.00 eq.), intermediate J, (310 mg, 0.77 mmol, 1.20 eq.), sodium bicarbonate (108 mg, 1.29 mmol, 2.00 eq.) and Pd(dtbpf)Cl₂ (42 mg, 0.064 mmol, 0.10 eq.) in dioxane (3 mL) and water (0.6 mL) was degassed with nitrogen and stirred at 80° C. for 1 hour. After such time the mixture was concentrated and the residue purified by prep-TLC (SiO₂, dichloromethane/methyl alcohol 20/1) to give tert-butyl N-[[7-[5-(4-chloro-2-cyano-3-methyl-phenyl)-1-methyl-pyrazol-4-yl]-4-oxo-3H-phthalazin-1-yl]methyl]carbamate, Intermediate 12-1 (150 mg, 0.30 mmol, 46% yield) as a yellow solid. LCMS [M+1]⁺=505.2.

Step 2: To a solution of tert-butyl ((7-(5-(4-chloro-2-cyano-3-methylphenyl)-1-methyl-1H-pyrazol-4-yl)-4-oxo-3,4-dihydrophthalazin-1-yl)methyl)carbamate (50 mg, 0.10 mmol, 1.00 eq.) in dichloromethane (1.5 mL) was added trifluoroacetic acid (0.4 mL). The mixture was stirred at 25° C. for 0.5 hour, concentrated in vacuum and the residue was purified by prep-HPLC (Phenomenex Gemini-NX C18 75×30 mm×3 μm; mobile phase: [water (0.1% TFA)-ACN]; B %: 22%-32%, 7 min) to give 6-(4-(4-(aminomethyl)-1-oxo-1,2-dihydrophthalazin-6-yl)-1-methyl-1H-pyrazol-5-yl)-3-chloro-2-methylbenzonitrile, Example 12-1 (14 mg, 0.026, mol, 27% yield) as a white solid. LCMS [M+1]⁺=405.2; ¹H NMR (400 MHz, DMSO-d₆) δ=12.87 (s, 1H), 8.55-8.34 (br s, 3H), 8.28 (s, 1H), 8.10 (d, J=8.4 Hz, 1H), 7.99 (d, J=8.4 Hz, 1H), 7.81 (s, 1H), 7.61 (d, J=8.4 Hz, 1H), 7.43 (d, J=8.4 Hz, 1H), 4.35 (br s, 2H), 3.73 (s, 3H), 2.58 (s, 3H).

### Example 12-2

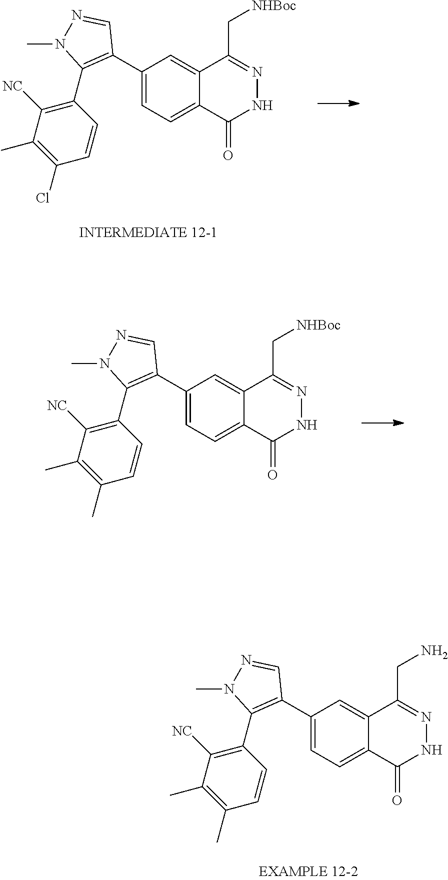 [SMILES: Cc1c(Cl)ccc(-c2c(-c3ccc4c(=O)[nH]nc(CNC(=O)OC(C)(C)C)c4c3)cnn2C)c1C#N ; Cc1ccc(-c2c(-c3ccc4c(=O)[nH]nc(CNC(=O)OC(C)(C)C)c4c3)cnn2C)c(C#N)c1C ; Cc1ccc(-c2c(-c3ccc4c(=O)[nH]nc(CN)c4c3)cnn2C)c(C#N)c1C]

Step 1: A mixture of Intermediate 12-1 (50 mg, 0.10 mmol, 1.00 eq.), methylboronic acid (18 mg, 0.30 mmol, 3.00 eq.), Pd(dtbpf)Cl₂ (6.5 mg, 0.010 mmol, 0.10 eq.) and potassium carbonate (41 mg, 0.30 mmol, 3.00 eq.) in dioxane (2 mL) was degassed with nitrogen and stirred at 100° C. for 1 hour. After such time the mixture was concentrated and the residue purified by prep-TLC (SiO₂, dichloromethane/methyl alcohol 20/1) to give tert-butyl N-[[7-[5-(2-cyano-3,4-dimethyl-phenyl)-1-methyl-pyrazol-4-yl]-4-oxo-3H-phthalazin-1-yl]methyl]carbamate (30 mg, 0.062 mmol, 63% yield) as a yellow solid. LCMS [M+1]⁺=485.3; ¹H NMR (400 MHz, DMSO-d₆) δ=10.22 (s, 1H), 8.25 (d, J=8.4 Hz, 1H), 7.99 (s, 1H), 7.60 (s, 1H), 7.57-7.46 (m, 2H), 7.20 (d, J=7.6 Hz, 1H), 5.42-5.33 (m, 1H), 3.81 (s, 3H), 3.50 (s, 2H), 2.59 (s, 3H), 2.46 (s, 3H), 1.49 (s, 9H).

Step 2: To a solution of tert-butyl N-[[7-[5-(2-cyano-3,4-dimethyl-phenyl)-1-methyl-pyrazol-4-yl]-4-oxo-3H-phthalazin-1-yl]methyl]carbamate (30 mg, 0.062 mmol, 1.00 eq.) in dichloromethane (1.5 mL) was added trifluoroacetic acid (0.5 mL). The mixture was stirred at 25° C. for 0.5 hour then concentrated in vacuum. The formed residue was purified by prep-HPLC (Column: Phenomenex luna C18 150×25 mm×10 μm; mobile phase: [water (0.1% TFA)-ACN]; B %: 6%-36%, 10 min) to give 6-(4-(4-(aminomethyl)-1-oxo-1,2-dihydrophthalazin-6-yl)-1-methyl-1H-pyrazol-5-yl)-2,3-dimethylbenzonitrile, Example 12-2 (7.8 mg, 0.016 mmol, 25% yield) as a white solid. LCMS [M+1]⁺=385.2; ¹H NMR (400 MHz, DMSO-d₆) δ=12.84 (s, 1H), 8.32 (br s, 3H), 8.26 (s, 1H), 8.08 (d, J=8.4 Hz, 1H), 7.74-7.65 (m, 2H), 7.46 (d, J=8.0 Hz, 2H), 4.26 (br d, J=1.2 Hz, 2H), 3.70 (s, 3H), 2.48 (br s, 3H), 2.42 (s, 3H).

### Example 12-3

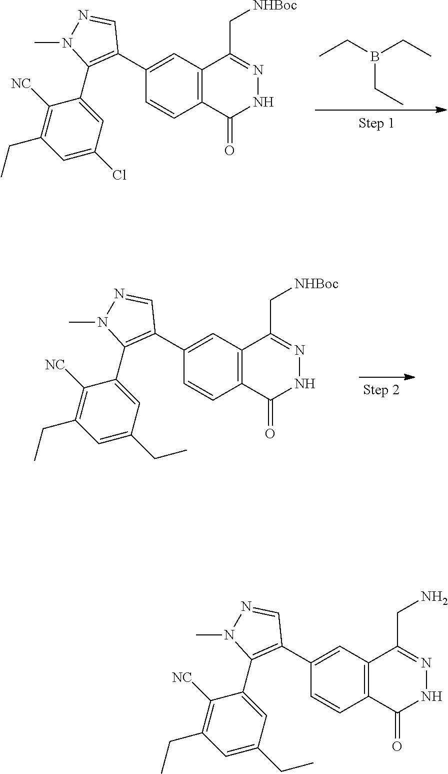

Step 1: To a solution of tert-butyl N-[[7-[5-(5-chloro-2-cyano-3-ethyl-phenyl)-1-methyl-pyrazol-4-yl]-4-oxo-3H-phthalazin-1-yl]methyl]carbamate (80 mg, 0.154 mmol, 1.00 eq.) and triethylborane (1.00 M, 0.450 mmol, 3.00 eq.) in THF (5 mL) and water (0.5 mL) was added potassium carbonate (64 mg, 0.462 mmol, 3.00 eq.) and XPhos Pd G3 (13 mg, 15 μmol, 0.10 eq.). The mixture was stirred at 85° C. for 8 hours then concentrated and the residue was diluted with water (10 mL) and extracted with ethyl acetate (20 mL×2). The combined organic extracts were washed with brine (20 mL), dried over anhydrous sodium sulfate, filtered and concentrated and the residue purified by prep-TLC (SiO₂, dichloromethane:methanol 20:1) to give tert-butyl N-[[7-[5-(2-cyano-3,5-diethyl-phenyl)-1-methyl-pyrazol-4-yl]-4-oxo-3H-phthalazin-1-yl]methyl]carbamate (40 mg crude) as a white solid. LCMS [M+1]⁺=513.5.

Step 2: To a mixture of tert-butyl N-[[7-[5-(2-cyano-3,5-diethyl-phenyl)-1-methyl-pyrazol-4-yl]-4-oxo-3H-phthalazin-1-yl]methyl]carbamate (40 mg, 78 μmol, 1.00 eq.) in dichloromethane (2 mL) was added trifluoroacetic acid (0.8 mL) in one portion at 0° C. under a nitrogen atmosphere. The mixture was stirred at 20° C. for 1 hour then concentrated under reduced pressure and the formed residue was purified by Prep-HPLC (Phenomenex luna C18 150×25 mm×10 μm; mobile phase: [water (0.1% TFA)-ACN]; B %: 17%-47%, 10 min) and further purified by SFC (DAICEL CHIRALCEL OD-H (250 mm×30 mm, 5 μm); mobile phase: [0.1% NH₃H₂O EtOH]; B %: 40%) to give 2-[4-[4-(aminomethyl)-1-oxo-2H-phthalazin-6-yl]-2-methyl-pyrazol-3-yl]-4,6-diethyl-benzonitrile, Example 12-3 (14 mg, 34 μmol, 70% yield) as a light yellow solid. LCMS [M+1]⁺=413.3; ¹H NMR (400 MHz, CDCl₃) δ=8.27 (br d, J=8.0 Hz, 1H), 7.93 (s, 1H), 7.54 (br d, J=8.4 Hz, 1H), 7.45 (s, 1H), 7.36 (s, 1H), 7.13 (s, 1H), 3.88 (br s, 2H), 3.82 (s, 3H), 2.91 (q, J=8.0 Hz, 2H), 2.74 (q, J=7.6 Hz, 2H), 2.12-1.99 (m, 2H), 1.32 (br t, J=7.6 Hz, 3H), 1.28-1.23 (m, 3H).

### Example 12-4

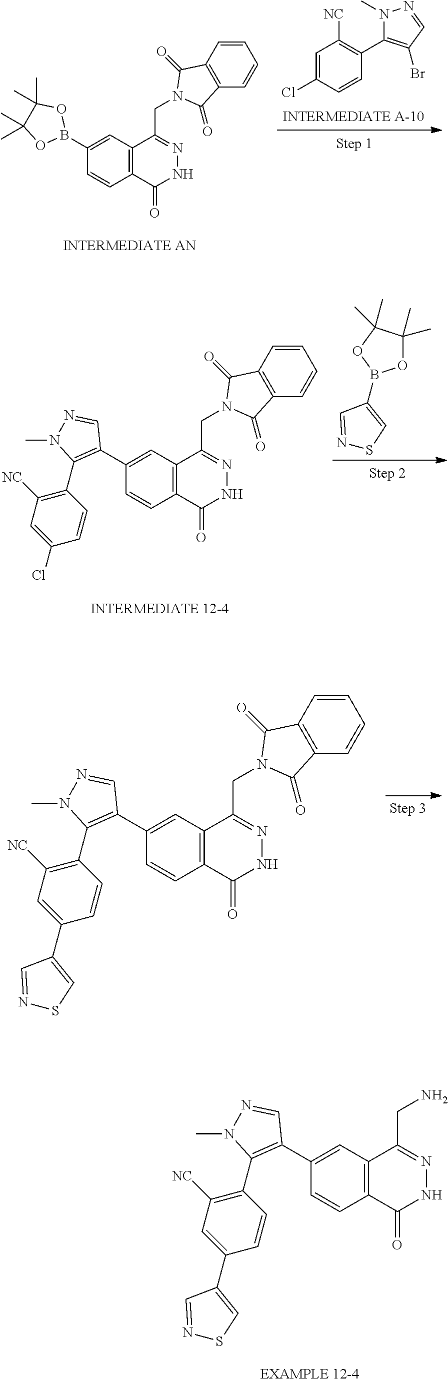 [SMILES: CC1(C)OB(c2ccc3c(=O)[nH]nc(CN4C(=O)c5ccccc5C4=O)c3c2)OC1(C)C ; Cn1ncc(Br)c1-c1ccc(Cl)cc1C#N ; Cn1ncc(-c2ccc3c(=O)[nH]nc(CN4C(=O)c5ccccc5C4=O)c3c2)c1-c1ccc(Cl)cc1C#N ; CC1(C)OB(c2cnsc2)OC1(C)C ; Cn1ncc(-c2ccc3c(=O)[nH]nc(CN4C(=O)c5ccccc5C4=O)c3c2)c1-c1ccc(-c2cnsc2)cc1C#N ; Cn1ncc(-c2ccc3c(=O)[nH]nc(CN)c3c2)c1-c1ccc(-c2cnsc2)cc1C#N]

Step 1: A mixture of 2-(4-bromo-2-methyl-pyrazol-3-yl)-5-chloro-benzonitrile, Intermediate A-10 (300 mg, 1.01 mmol, 1.00 eq.), 2-[[4-oxo-7-(4,4,5,5-tetramethyl-1,3,2-dioxaborolan-2-yl)-3H-phthalazin-1-yl]methyl]isoindoline-1,3-dione, Intermediate J (523 mg, 1.21 mmol, 1.20 eq.), di-tert-butyl(cyclopentyl)phosphane-dichloropalladium iron (66 mg, 0.101 mmol, 0.10 eq.), sodium bicarbonate (170 mg, 2.02 mmol, 79 μL, 2.00 eq.) in 1,4-dioxane (10 mL) and water (2 mL) was degassed and purged with nitrogen 3 times then stirred at 80° C. for 2 hours. After such time the reaction mixture was quenched by the addition of water (30 mL) at 20° C. then extracted with dichloromethane (30 mL×2). The combined organic layers were washed with brine (30 mL), dried over anhydrous sodium sulfate, filtered and concentrated under reduced pressure to give 5-chloro-2-[4-[4-[(1,3-dioxoisoindolin-2-yl)methyl]-1-oxo-2H-phthalazin-6-yl]-2-methyl-pyrazol-3-yl]benzonitrile, Intermediate 12-4 (300 mg, crude) as brown liquid which used into the next step without further purification. LCMS [M+1]⁺=521.1

Step 2: A mixture of 4-(4,4,5,5-tetramethyl-1,3,2-dioxaborolan-2-yl)isothiazole (39 mg, 0.184 mmol, 1.20 eq.), 5-chloro-2-[4-[4-[(1,3-dioxoisoindolin-2-yl)methyl]-1-oxo-2H-phthalazin-6-yl]-2-methyl-pyrazol-3-yl]benzonitrile (80 mg, 0.153 mmol, 1.00 eq.), Xphos precatalyst G2 (36 mg, 0.046 mmol, 0.30 eq.) and potassium phosphate (65 mg, 0.307 mmol, 2.00 eq.) in 1,4-dioxane (4 mL) and water (0.4 mL) was degassed and purged with nitrogen 3 times then stirred at 110° C. for 1 hour. After such time the reaction was cooled to room temperature, diluted with dichloromethane (20 mL), washed with water (5 mL×2) and the organic layer was dried over anhydrous sodium sulfate, filtered and concentrated to give 2-[4-[4-[(1,3-dioxoisoindolin-2-yl)methyl]-1-oxo-2H-phthalazin-6-yl]-2-methyl-pyrazol-3-yl]-5-isothiazol-4-yl-benzonitrile (66 mg, crude) as a brown oil which used into the next step without further purification. LCMS [M+1]⁺=570.1.

Step 3: To a solution of 2-[4-[4-[(1,3-dioxoisoindolin-2-yl)methyl]-1-oxo-2H-phthalazin-6-yl]-2-methyl-pyrazol-3-yl]-5-isothiazol-4-yl-benzonitrile (60 mg, 0.105 mmol, 1.00 eq.) in 1,4-dioxane (5 mL) was added hydrazine hydrate (53 mg, 1.05 mmol, 51 μL, 10 eq.). The mixture was stirred at 20° C. for 15 hours then concentrated under reduced pressure. The residue was purified by prep-HPLC (YMC-Actus Triart C18 150×30 mm×5 μm; mobile phase: [water (0.1% TFA)-ACN]; B %: 17%-43%, 11 min) to give 2-[4-[4-(aminomethyl)-1-oxo-2H-phthalazin-6-yl]-2-methyl-pyrazol-3-yl]-5-isothiazol-4-yl-benzonitrile, Example 12-4 (6 mg, 14% yield) as a white solid. LCMS [M+1]⁺=440.0; ¹H NMR (400 MHz, DMSO-d₆) δ=12.87 (s, 1H), 9.69 (s, 1H), 9.27 (s, 1H), 8.59 (d, J=1.6 Hz, 1H), 8.37 (br d, J=2.0 Hz, 2H), 8.40-8.24 (m, 4H), 8.11 (d, J=8.4 Hz, 1H), 7.89 (d, J=8.0 Hz, 1H), 7.85-7.78 (m, 1H), 7.45 (dd, J=1.6, 8.0 Hz, 1H), 4.39-4.26 (m, 2H), 3.78 (s, 3H).

### Example 12-5

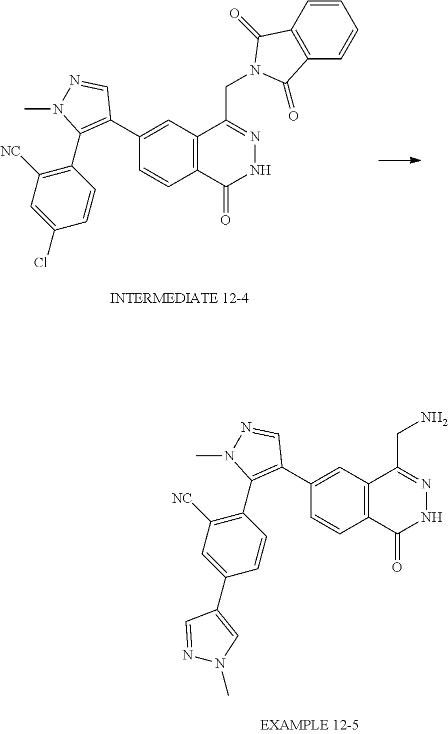 [SMILES: Cn1ncc(-c2ccc3c(=O)[nH]nc(CN4C(=O)c5ccccc5C4=O)c3c2)c1-c1ccc(Cl)cc1C#N ; Cn1cc(-c2ccc(-c3c(-c4ccc5c(=O)[nH]nc(CN)c5c4)cnn3C)c(C#N)c2)cn1]

Example 12-5, 2-(4-(4-(aminomethyl)-1-oxo-1,2-dihydrophthalazin-6-yl)-1-methyl-1H-pyrazol-5-yl)-5-(1-methyl-1H-pyrazol-4-yl)benzonitrile was prepared from 5-chloro-2-[4-[4-[(1,3-dioxoisoindolin-2-yl)methyl]-1-oxo-2H-phthalazin-6-yl]-2-methyl-pyrazol-3-yl]benzonitrile, Intermediate 12-4 and 1-methyl-4-(4,4,5,5-tetramethyl-1,3,2-dioxaborolan-2-yl)pyrazole following the procedure described for the preparation of Example 12-4, steps 2 and 3 as a white solid (6 mg, 13 μmol, 25% yield). LCMS [M+1]⁺=437.2. ¹H NMR (400 MHz, CD₃OD) δ=8.24 (s, 1H), 8.19 (s, 1H), 8.16 (d, J=1.6 Hz, 1H), 8.12 (s, 1H), 8.09-8.04 (m, 1H), 8.01 (s, 1H), 7.72-7.64 (m, 3H), 3.96 (s, 3H), 3.88 (s, 2H), 3.83 (s, 3H).

### Example 12-6

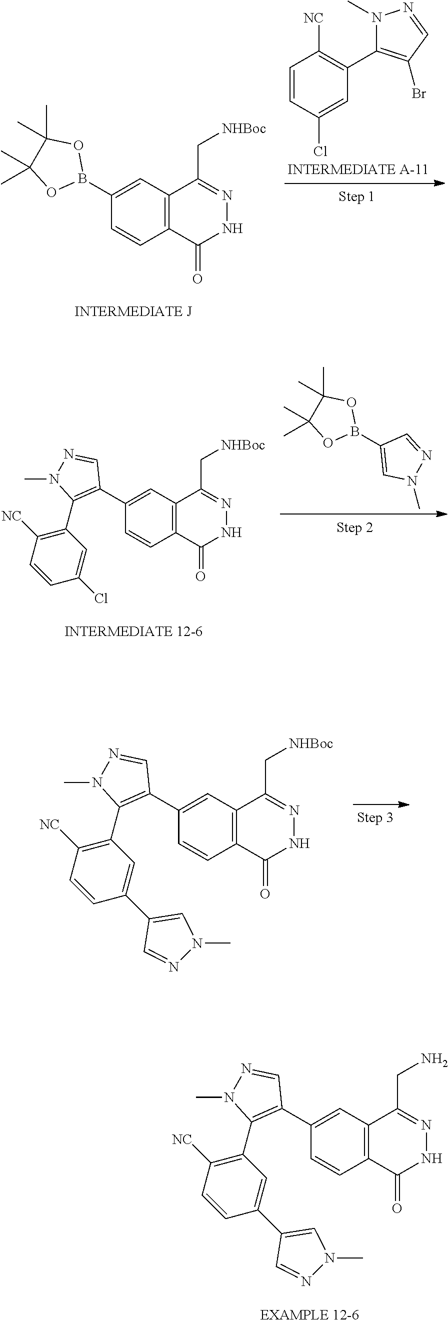 [SMILES: CC(C)(C)OC(=O)NCc1n[nH]c(=O)c2ccc(B3OC(C)(C)C(C)(C)O3)cc12 ; Cn1ncc(Br)c1-c1cc(Cl)ccc1C#N ; Cn1ncc(-c2ccc3c(=O)[nH]nc(CNC(=O)OC(C)(C)C)c3c2)c1-c1cc(Cl)ccc1C#N ; Cn1cc(B2OC(C)(C)C(C)(C)O2)cn1 ; Cn1cc(-c2ccc(C#N)c(-c3c(-c4ccc5c(=O)[nH]nc(CNC(=O)OC(C)(C)C)c5c4)cnn3C)c2)cn1 ; Cn1cc(-c2ccc(C#N)c(-c3c(-c4ccc5c(=O)[nH]nc(CN)c5c4)cnn3C)c2)cn1]

Step 1: To a solution of 2-(4-bromo-2-methyl-pyrazol-3-yl)-4-chloro-benzonitrile, Intermediate A-11 (160 mg, 0.539 mmol, 1.00 eq.), sodium bicarbonate (91 mg, 1.08 mmol, 41.9 μL, 2.00 eq.) and tert-butyl-N-[[4-oxo-7-(4,4,5,5-tetramethyl-1,3,2-dioxaborolan-2-yl)-3H-phthalazin-1-yl]methyl]carbamate, Intermediate J (281 mg, 0.701 mmol, 1.30 eq.) in 1,4-dioxane (5 mL) and water (1 mL) was added di-tert-butyl(cyclopentyl)phosphane-dichloropalladium iron (35 mg, 54 μmol, 0.10 eq.) and the mixture was stirred at 80° C. for 2 hours under nitrogen. After such time the reaction mixture was partitioned between ethyl acetate (10 mL) and water (10 mL). The organic phase was separated, washed with brine (10 mL), dried over anhydrous sodium sulfate, filtered and concentrated. The residue was purified by flash silica gel chromatography (50 to 100% petroleum ether:ethyl acetate gradient) to give tert-butyl N-[[7-[5-(5-chloro-2-cyano-phenyl)-1-methyl-pyrazol-4-yl]-4-oxo-3H-phthalazin-1-yl]methyl]carbamate, Intermediate 12-6 (160 mg, 0.313 mmol, 58% yield) as a gray solid. LCMS [M+1]⁺=491.2.

Step 2: To a solution of tert-butyl N-[[7-[5-(5-chloro-2-cyano-phenyl)-1-methyl-pyrazol-4-yl]-4-oxo-3H-phthalazin-1-yl]methyl]carbamate (50 mg, 0.102 mmol, 1.00 eq.), potassium carbonate (42 mg, 0.305 mmol, 3.00 eq.) and 1-methyl-4-(4,4,5,5-tetramethyl-1,3,2-dioxaborolan-2-yl)pyrazole (42 mg, 0.204 mmol, 2.00 eq.) in 1,4-dioxane (1.0 mL) and water (0.1 mL) was added di-tert-butyl(cyclopentyl)phosphane dichloropalladium iron (13 mg, 20.4 μmol, 0.20 eq.) and the mixture was stirred at 80° C. for 3 hours under nitrogen. After such time the reaction was cooled to 10° C. and the salt was removed by filtration and the filtrate concentrated. The residue was purified by prep-TLC (SiO₂, petroleum ether:ethyl acetate 1:1.5) to give tert-butyl N-[[7-[5-[2-cyano-5-(1-methylpyrazol-4-yl)phenyl]-1-methyl-pyrazol-4-yl]-4-oxo-3H-phthalazin-1-yl]methyl]carbamate (40 mg, 64 μmol, 63% yield) as a white solid. LCMS [M+1]⁺=537.3.

Step 3: To a solution of tert-butyl N-[[7-[5-[2-cyano-5-(1-methylpyrazol-4-yl)phenyl]-1-methyl-pyrazol-4-yl]-4-oxo-3H-phthalazin-1-yl]methyl]carbamate (37 mg, 63 μmol, 1.00 eq.) in methanol (2 mL) was added a solution of hydrogen chloride in 1,4-dioxane (4 M, 2 mL) and the mixture was stirred at 25° C. for 2 hours. After such time the reaction mixture was concentrated and the residue was purified by prep-HPLC (Phenomenex Synergi C18 150×30 mm×4 μm; mobile phase: [water (0.05% HCl)-ACN]; B %: 15%-35%, 9 min) to give 2-[4-[4-(aminomethyl)-1-oxo-2H-phthalazin-6-yl]-2-methyl-pyrazol-3-yl]-4-(1-methylpyrazol-4-yl)benzonitrile, Example 12-6 (7 mg, 16 μmol, 23% yield) as a white solid. LCMS [M+1]⁺=437.2; ¹H NMR (400 MHz, CD₃OD) δ=8.26 (s, 1H), 8.21-8.13 (m, 2H), 8.09-8.04 (m, 1H), 8.01-7.94 (m, 2H), 7.92-7.87 (m, 1H), 7.74 (d, J=1.2 Hz, 1H), 7.58-7.55 (m, 1H), 4.38 (s, 2H), 3.96 (s, 3H), 3.85 (s, 3H).

### Example 12-7

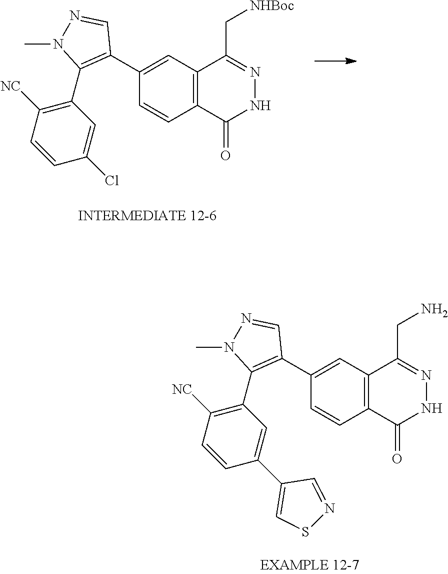 [SMILES: Cn1ncc(-c2ccc3c(=O)[nH]nc(CNC(=O)OC(C)(C)C)c3c2)c1-c1cc(Cl)ccc1C#N ; Cn1ncc(-c2ccc3c(=O)[nH]nc(CN)c3c2)c1-c1cc(-c2cnsc2)ccc1C#N]

Example 12-7, 2-(4-(4-(aminomethyl)-1-oxo-1,2-dihydrophthalazin-6-yl)-1-methyl-1H-pyrazol-5-yl)-4-(isothiazol-4-yl)benzonitrile was prepared from tert-butyl N-[[7-[5-(5-chloro-2-cyano-phenyl)-1-methyl-pyrazol-4-yl]-4-oxo-3H-phthalazin-1-yl]methyl]carbamate, Intermediate 12-6 and 4-(4,4,5,5-tetramethyl-1,3,2-dioxaborolan-2-yl)isothiazole following the procedure described for the preparation of Example 12-6, steps 2 and 3 as a white solid (10 mg, 23 μmol, 21% yield). LCMS [M+1]⁺=440.2. ¹H NMR (400 MHz, CD₃OD) δ=9.43 (s, 1H), 9.05 (s, 1H), 8.21-8.14 (m, 4H), 8.00 (d, J=8.0 Hz, 1H), 7.77 (d, J=1.2 Hz, 1H), 7.58-7.56 (m, 1H), 4.46-4.33 (m, 2H), 3.87 (s, 3H).

### Example 12-8

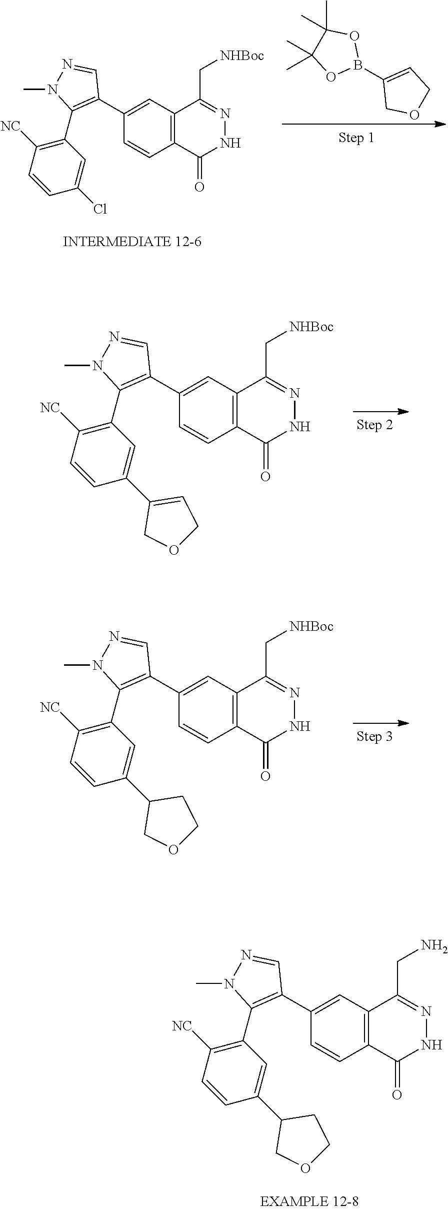 [SMILES: Cn1ncc(-c2ccc3c(=O)[nH]nc(CNC(=O)OC(C)(C)C)c3c2)c1-c1cc(Cl)ccc1C#N ; CC1(C)OB(C2=CCOC2)OC1(C)C ; Cn1ncc(-c2ccc3c(=O)[nH]nc(CNC(=O)OC(C)(C)C)c3c2)c1-c1cc(C2=CCOC2)ccc1C#N ; Cn1ncc(-c2ccc3c(=O)[nH]nc(CNC(=O)OC(C)(C)C)c3c2)c1-c1cc(C2CCOC2)ccc1C#N ; Cn1ncc(-c2ccc3c(=O)[nH]nc(CN)c3c2)c1-c1cc(C2CCOC2)ccc1C#N]

Step 1: To a solution of tert-butyl N-[[7-[5-(5-chloro-2-cyano-phenyl)-1-methyl-pyrazol-4-yl]-4-oxo-3H-phthalazin-1-yl]methyl]carbamate, Intermediate 12-6 (100 mg, 0.204 mmol, 1.00 eq.), potassium carbonate (56 mg, 0.407 mmol, 2.00 eq.) and 2-(2,5-dihydrofuran-3-yl)-4,4,5,5-tetramethyl-1,3,2-dioxaborolane (80 mg, 0.407 mmol, 2.00 eq.) in 1,4-dioxane (5 mL) and water (1 mL) was added di-tert-butyl(cyclopentyl)phosphanedichloropalladium iron (26.6 mg, 40.7 μmol, 0.20 eq.). The mixture was stirred at 90° C. for 16 hours then cooled to 10° C. and the salts was removed by filtration. The filtrate was diluted with water (10 mL) and extracted with ethyl acetate (10 mL×2) and the combined organic layers were washed with brine (20 mL), dried over anhydrous magnesium sulfate, filtered and concentrated. The residue was purified by prep-TLC (SiO₂, dichloromethane:methanol 20:1) to give tert-butyl N-[[7-[5-[2-cyano-5-(2,5-dihydrofuran-3-yl)phenyl]-1-methyl-pyrazol-4-yl]-4-oxo-3H-phthalazin-1-yl]methyl]carbamate (0.08 g, 0.114 mmol, 56% yield) as a gray solid. LCMS [M+1]⁺=525.3.

Step 2: To a solution of tert-butyl N-[[7-[5-[2-cyano-5-(2,5-dihydrofuran-3-yl)phenyl]-1-methyl-pyrazol-4-yl]-4-oxo-3H-phthalazin-1-yl]methyl]carbamate (80 mg, 0.114 mmol, 1.00 eq.) in methanol (20 mL) was added palladium on activated carbon (6.6 mg, 11 μmol, 10% Pd, 0.10 eq.) and the mixture was vigorously stirred at 25° C. for 2 hours. After such time the mixture was filtered and the filtrated was concentrated to give tert-butyl N-[[7-[5-(2-cyano-5-THF-3-yl-phenyl)-1-methyl-pyrazol-4-yl]-4-oxo-3H-phthalazin-1-yl]methyl]carbamate (75 mg, 105 μmol, 92% yield) as a white solid which used directly for the next step without further purification. LCMS [M+1]⁺=527.3

Step 3: To a solution of tert-butyl N-[[7-[5-(2-cyano-5-THF-3-yl-phenyl)-1-methyl-pyrazol-4-yl]-4-oxo-3H-phthalazin-1-yl]methyl]carbamate (74.0 mg, 105 μmol, 1.00 eq.) in dichloromethane (2 mL) was added a solution of hydrogen chloride in 1,4-dioxane (4.00 M, 0.26 mL, 10.0 eq.). The mixture was stirred at 25° C. for 1 hr then concentrated under reduced pressure and the residue purified by prep-HPLC (Welch Xtimate C18 150×25 mm×5 μm; mobile phase: [water (10 mM NH₄HCO₃)-ACN]; B %: 12%-42%, 10 min) to give 2-[4-[4-(aminomethyl)-1-oxo-2H-phthalazin-6-yl]-2-methyl-pyrazol-3-yl]-4-THF-3-yl-benzonitrile, Example 12-8 (6 mg, 13 μmol, 12% yield) as a white solid. LCMS [M+1]⁺=427.3; ¹H NMR (400 MHz, DMSO) δ=12.37 (s, 1H), 8.17 (d, J=0.8 Hz, 1H), 8.09 (d, J=8.4 Hz, 1H), 7.97 (d, J=8.0 Hz, 1H), 7.70-7.64 (m, 3H), 7.47 (d, J=1.2 Hz, 1H), 4.05-3.84 (m, 3H), 3.79-3.72 (m, 1H), 3.70 (s, 3H), 3.65-3.48 (m, 5H), 2.39-2.26 (m, 1H), 2.01-1.87 (m, 1H).

### Example 12-9

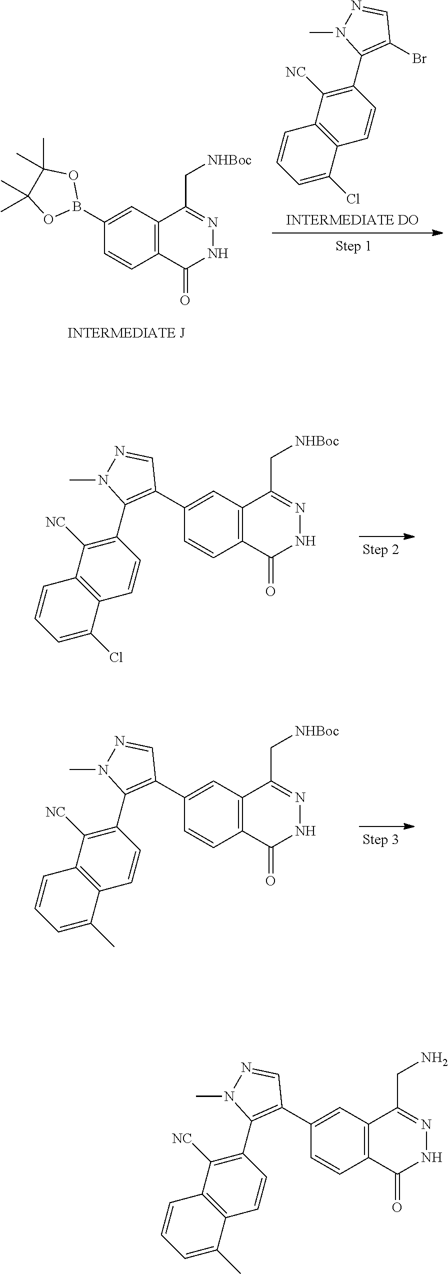 [SMILES: CC(C)(C)OC(=O)NCc1n[nH]c(=O)c2ccc(B3OC(C)(C)C(C)(C)O3)cc12 ; Cn1ncc(Br)c1-c1ccc2c(Cl)cccc2c1C#N ; Cn1ncc(-c2ccc3c(=O)[nH]nc(CNC(=O)OC(C)(C)C)c3c2)c1-c1ccc2c(Cl)cccc2c1C#N ; Cc1cccc2c(C#N)c(-c3c(-c4ccc5c(=O)[nH]nc(CNC(=O)OC(C)(C)C)c5c4)cnn3C)ccc12 ; Cc1cccc2c(C#N)c(-c3c(-c4ccc5c(=O)[nH]nc(CN)c5c4)cnn3C)ccc12]

Step 1: A mixture of 2-(4-bromo-1-methyl-1H-pyrazol-5-yl)-5-chloro-1-naphthonitrile, Intermediate DO, (130 mg, 0.375 mmol, 1.00 eq.), tert-butyl N-[[4-oxo-7-(4,4,5,5-tetramethyl-1,3,2-dioxaborolan-2-yl)-3H-phthalazin-1-yl]methyl]carbamate, Intermediate J (181 mg, 0.45 mmol, 1.20 eq.), Pd(dtbpf)Cl₂ (24 mg, 38 μmol, 0.10 eq.), sodium carbonate (80 mg, 0.75 mmol, 2.00 eq.) in the mixed solvents dioxane (5 mL) and water (1 mL) was degassed with nitrogen and stirred at 80° C. for 1 hour under nitrogen atmosphere. After such time the mixture was concentrated and the residue was purified by prep-TLC (SiO₂, dichloromethane:methyl alcohol 20:1) to give tert-butyl N-[[7-[5-(5-chloro-1-cyano-2-naphthyl)-1-methyl-pyrazol-4-yl]-4-oxo-3H-phthalazin-1-yl]methyl]carbamate (120 mg, 0.222 mmol, 59% yield) as a white solid. LCMS [M+1]⁺=541.1.

Step 2: A mixture of tert-butyl N-[[7-[5-(5-chloro-1-cyano-2-naphthyl)-1-methyl-pyrazol-4-yl]-4-oxo-31H-phthalazin-1-yl]methyl]carbamate (70 mg, 129 μmol, 1.00 eq.), methylboronic acid (23 mg, 0.388 mmol, 3.00 eq.), Pd(dtbpf)Cl₂ (8.4 mg, 12 μmol, 0.10 eq.), potassium carbonate (36 mg, 259 μmol, 2.00 eq.) in dioxane (2 mL) was degassed and purged with nitrogen and stirred at 100° C. for 1 hour under nitrogen atmosphere. The mixture was concentrated and purified by prep-TLC (SiO₂, dichloromethane:methyl alcohol 20:1) to give tert-butyl N-[[7-[5-(1-cyano-5-methyl-2-naphthyl)-1-methyl-pyrazol-4-yl]-4-oxo-3H-phthalazin-1-yl]methyl]carbamate (50 mg, 96 μmol, 74% yield) as a white solid. LCMS [M+1]⁺=521.5.

Step 3: To a solution of tert-butyl N-[[7-[5-(1-cyano-5-methyl-2-naphthyl)-1-methyl-pyrazol-4-yl]-4-oxo-3H-phthalazin-1-yl]methyl]carbamate (50 mg, 96 μmol, 1.00 eq.) in dichloromethane (1.5 mL) was added trifluoroacetic acid (417 μL) and the mixture stirred at 25° C. for 0.5 hour. After such time the mixture was concentrated and the residue was purified by prep-HPLC (Phenomenex Luna C18 75×30 mm×3 μm; mobile phase: [water (0.05% HCl)-ACN]; B %: 17%-37%, 7 min) to give 2-(4-(4-(aminomethyl)-1-oxo-1,2-dihydrophthalazin-6-yl)-1-methyl-1H-pyrazol-5-yl)-5-methyl-1-naphthonitrile, Example 12-9 (21 mg, 45 μmol, 47% yield, HCl) as a white solid. LCMS [M+1]⁺=421.1; ¹H NMR (400 MHz, DMSO-d₆) δ=12.83 (s, 1H), 8.59 (dd, J=0.8, 8.8 Hz, 1H), 8.45 (br s, 3H), 8.35 (s, 1H), 8.05-7.96 (m, 2H), 7.92 (d, J=1.6 Hz, 1H), 7.85 (d, J=8.8 Hz, 1H), 7.81-7.74 (m, 1H), 7.68 (d, J=7.2 Hz, 1H), 7.31 (dd, J=1.6, 8.4 Hz, 1H), 4.44-4.17 (m, 2H), 3.77 (s, 3H), 2.79 (s, 3H).

### Example 12-10

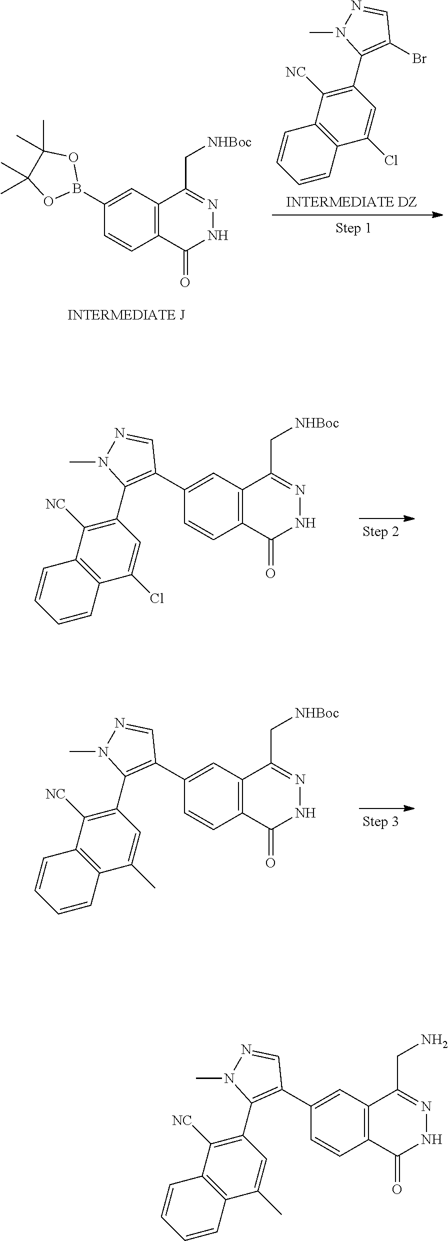 [SMILES: CC(C)(C)OC(=O)NCc1n[nH]c(=O)c2ccc(B3OC(C)(C)C(C)(C)O3)cc12 ; Cn1ncc(Br)c1-c1cc(Cl)c2ccccc2c1C#N ; Cn1ncc(-c2ccc3c(=O)[nH]nc(CNC(=O)OC(C)(C)C)c3c2)c1-c1cc(Cl)c2ccccc2c1C#N ; Cc1cc(-c2c(-c3ccc4c(=O)[nH]nc(CNC(=O)OC(C)(C)C)c4c3)cnn2C)c(C#N)c2ccccc12 ; Cc1cc(-c2c(-c3ccc4c(=O)[nH]nc(CN)c4c3)cnn2C)c(C#N)c2ccccc12]

Example 12-10, 2-(4-(4-(aminomethyl)-1-oxo-1,2-dihydrophthalazin-6-yl)-1-methyl-1H-pyrazol-5-yl)-4-methyl-1-naphthonitrile was prepared from Intermediate J (346 mg, 0.863 mmol, 1.30 eq.), and Intermediate DZ (230 mg, 0.664 μmol, 1.00 eq.), according to the procedure described for the synthesis of Example 12-9 as a yellow solid (20 mg, 0.042 mmol, final step 55% yield). LCMS [M+1]⁺=421.1; ¹H NMR (400 MHz, DMSO-d₆) δ=12.83 (s, 1H), 8.55-8.43 (m, 3H), 8.36 (s, 1H), 8.36-8.32 (m, 1H), 8.18-8.12 (m, 1H), 8.01 (d, J=8.4 Hz, 1H), 7.93-7.86 (m, 3H), 7.75 (d, J=0.8 Hz, 1H), 7.37 (dd, J=1.6, 8.4 Hz, 1H), 4.45-4.11 (m, 2H), 3.78 (s, 3H), 2.86 (s, 3H).

### Example 12-11

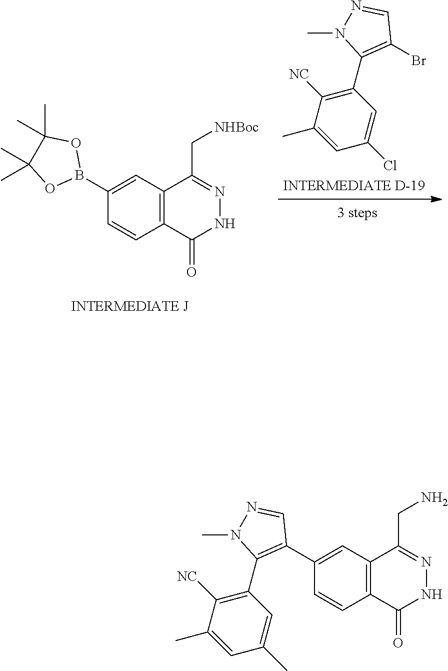 [SMILES: CC(C)(C)OC(=O)NCc1n[nH]c(=O)c2ccc(B3OC(C)(C)C(C)(C)O3)cc12 ; Cc1cc(Cl)cc(-c2c(Br)cnn2C)c1C#N ; Cc1cc(C)c(C#N)c(-c2c(-c3ccc4c(=O)[nH]nc(CN)c4c3)cnn2C)c1]

2-(4-(4-(aminomethyl)-1-oxo-1,2-dihydrophthalazin-6-yl)-1-methyl-1H-pyrazol-5-yl)-4,6-dimethylbenzonitrile, Example 12-11 was prepared using the same method as Example 12-9 starting from Intermediate DO for Intermediate D-19, as a white solid (7.6 mg, 0.015 mmol, 37% yield). LCMS [M+1]⁺=385.2; ¹H NMR (400 MHz, DMSO-d₆) δ=12.89-12.85 (m, 1H), 8.36 (br s, 3H), 8.28 (s, 1H), 8.10 (d, J=8.4 Hz, 1H), 7.69 (d, J=1.6 Hz, 1H), 7.52 (s, 1H), 7.49 (dd, J=1.6, 8.4 Hz, 1H), 7.41 (s, 1H), 4.35-4.11 (m, 2H), 3.71 (s, 3H), 2.52-2.51 (m, 3H), 2.44 (s, 3H).

### Example 12-12

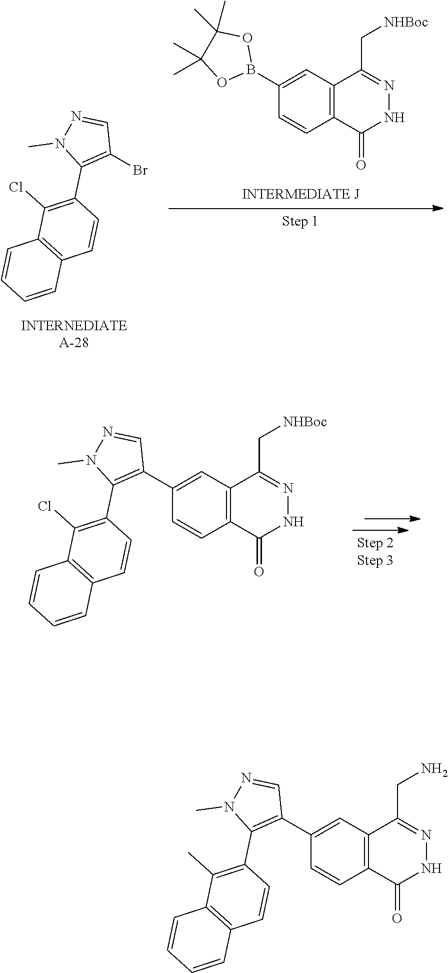 [SMILES: Cn1ncc(Br)c1-c1ccc2ccccc2c1Cl ; CC(C)(C)OC(=O)NCc1n[nH]c(=O)c2ccc(B3OC(C)(C)C(C)(C)O3)cc12 ; Cn1ncc(-c2ccc3c(=O)[nH]nc(CNC(=O)OC(C)(C)C)c3c2)c1-c1ccc2ccccc2c1Cl ; Cc1c(-c2c(-c3ccc4c(=O)[nH]nc(CN)c4c3)cnn2C)ccc2ccccc12]

Step 1: A mixture of 4-bromo-5-(1-chloro-2-naphthyl)-1-methyl-pyrazole (1.20 g, 3.73 mmol, 1.00 eq.), tert-butyl N-[[4-oxo-7-(4,4,5,5-tetramethyl-1,3,2-dioxaborolan-2-yl)-3H-phthalazin-1-yl]methyl]carbamate (1.50 g, 3.73 mmol, 1.00 eq.), sodium bicarbonate (627 mg, 7.46 mmol, 2.00 eq.) and di-tert-butyl(cyclopentyl)phosphane-dichloropalladium;iron (243 mg, 0.373 mmol, 0.10 eq.) in dioxane (10 mL) and water (2 mL) was degassed and purged with nitrogen 3 times, and stirred at 80° C. for 0.5 hour. The reaction mixture was then filtered and concentrated and the residue was diluted with water (30 mL) and extracted with ethyl acetate (30 mL×2), dried over anhydrous sodium sulfate, filtered and concentrated and the residue was purified by column chromatography (SiO₂, Petroleum ether:Ethyl acetate 10-1000%) to give tert-butyl N-[[7-[5-(1-chloro-2-naphthyl)-1-methyl-pyrazol-4-yl]-4-oxo-3H-phthalazin-1-yl]methyl]carbamate (1.20 g, 2.33 mmol, 62% yield) as a white solid. LCMS [M+1]⁺=516.1; ¹H NMR (400 MHz, CDCl₃) δ=10.08 (s, 1H), 8.41 (d, J=8.0 Hz, 1H), 8.18-8.10 (m, 1H), 8.04 (s, 1H), 7.96 (dd, J=8.4, 16.0 Hz, 2H), 7.84-7.77 (m, 1H), 7.76-7.66 (m, 2H), 7.47-7.39 (m, 1H), 7.36 (d, J=8.8 Hz, 1H), 5.27-5.15 (m, 1H), 4.46-4.38 (m, 1H), 4.33-4.26 (m, 1H), 3.77 (s, 3H), 1.49 (s, 9H).

Step 2: To a solution of tert-butyl N-[[7-[5-(1-chloro-2-naphthyl)-1-methyl-pyrazol-4-yl]-4-oxo-3H-phthalazin-1-yl]methyl]carbamate (100 mg, 0.194 mmol, 1.00 eq.) in toluene (2 mL) was added potassium phosphate (123 mg, 0.581 mmol, 3.00 eq.), 2,4,6-trimethyl-1,3,5,2,4,6-trioxatriborinane (292 mg, 1.16 mmol, 50% purity, 6.00 eq.) and SPhos Pd G2 (15 mg, 0.194 mmol, 0.10 eq.) and the mixture was stirred at 100° C. for 10 hours under nitrogen atmosphere. After such time the mixture was filtered, diluted with water (5 mL) and extracted with ethyl acetate (5 mL) and the organic layer dried over anhydrous sodium sulfate, filtered and concentrated under reduced pressure to give tert-butyl N-[[7-[1-methyl-5-(1-methyl-2-naphthyl)pyrazol-4-yl]-4-oxo-3H-phthalazin-1-yl]methyl]carbamate (150 mg, crude) as a yellow solid. LCMS [M+1]⁺=496.5.

Step 3: To a solution of tert-butyl N-[[7-[1-methyl-5-(1-methyl-2-naphthyl)pyrazol-4-yl]-4-oxo-3H-phthalazin-1-yl]methyl]carbamate (150 mg, 0.303 mmol, 1.00 eq.) in the dichloromethane (1 mL) was added trifluoroacetic acid (462 mg, 4.05 mmol, 0.30 mL, 13.4 eq.). The mixture was then stirred at 25° C. for 0.5 hour then concentrated and the residue purified by prep-HPLC (Phenomenex Synergi C18 150×30 mm×4 μm; mobile phase: [water (0.1% TFA)-ACN]; B %: 23%-43%, 10 min) to give 4-(aminomethyl)-6-[1-methyl-5-(1-methyl-2-naphthyl)pyrazol-4-yl]-2H-phthalazin-1-one (16 mg, 0.038. mmol, 13% yield) as an off-white solid. LCMS [M+1]⁺=396.1; ¹H NMR (500 MHz, MeOD) δ=8.23-8.21 (m, 1H), 8.19-8.13 (m, 2H), 8.03-7.96 (m, 2H), 7.73-7.69 (m, 1H), 7.68-7.64 (m, 2H), 7.60-7.58 (m, 1H), 7.41 (d, J=8.5 Hz, 1H), 4.14 (d, J=13.5 Hz, 1H), 3.98 (d, J=13.0 Hz, 1H), 3.70 (s, 3H), 2.43 (s, 3H).

### Example 12-13

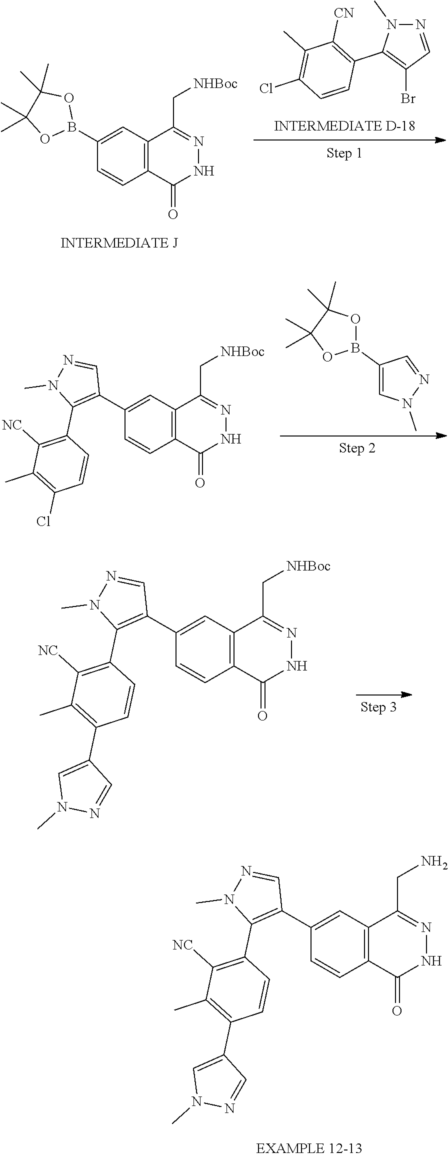 [SMILES: CC(C)(C)OC(=O)NCc1n[nH]c(=O)c2ccc(B3OC(C)(C)C(C)(C)O3)cc12 ; Cc1c(Cl)ccc(-c2c(Br)cnn2C)c1C#N ; Cc1c(Cl)ccc(-c2c(-c3ccc4c(=O)[nH]nc(CNC(=O)OC(C)(C)C)c4c3)cnn2C)c1C#N ; Cn1cc(B2OC(C)(C)C(C)(C)O2)cn1 ; Cc1c(-c2cnn(C)c2)ccc(-c2c(-c3ccc4c(=O)[nH]nc(CNC(=O)OC(C)(C)C)c4c3)cnn2C)c1C#N ; Cc1c(-c2cnn(C)c2)ccc(-c2c(-c3ccc4c(=O)[nH]nc(CN)c4c3)cnn2C)c1C#N]

### Example 12-13

Example 12-13, 6-(4-(4-(aminomethyl)-1-oxo-1,2-dihydrophthalazin-6-yl)-1-methyl-1H-pyrazol-5-yl)-2-methyl-3-(1-methyl-1H-pyrazol-4-yl)benzonitrile was prepared as a white solid (10 mg, 0.018 mmol, 33% yield), starting with Intermediate J and Intermediate D-18 following the same procedure as described for Example 12-6. LCMS [M+1]⁺=451.2; ¹H NMR (500 MHz, DMSO-d₆) δ=12.86 (s, 1H), 8.33 (br s, 3H), 8.27 (s, 1H), 8.16 (s, 1H), 8.10 (d, J=8.5 Hz, 1H), 7.88 (d, J=8.5 Hz, 1H), 7.86 (d, J=0.5 Hz, 1H), 7.78 (d, J=1.5 Hz, 1H), 7.58 (d, J=8.5 Hz, 1H), 7.48 (dd, J=1.5, 8.5 Hz, 1H), 4.31 (br t, J=6.0 Hz, 2H), 3.93 (s, 3H), 3.74 (s, 3H), 2.62 (s, 3H).

### Example 12-14

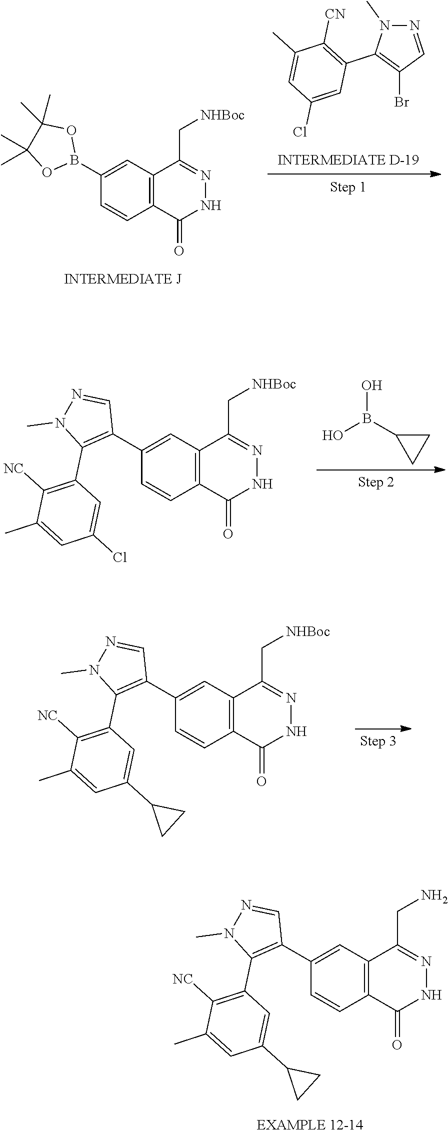 [SMILES: CC(C)(C)OC(=O)NCc1n[nH]c(=O)c2ccc(B3OC(C)(C)C(C)(C)O3)cc12 ; Cc1cc(Cl)cc(-c2c(Br)cnn2C)c1C#N ; Cc1cc(Cl)cc(-c2c(-c3ccc4c(=O)[nH]nc(CNC(=O)OC(C)(C)C)c4c3)cnn2C)c1C#N ; OB(O)C1CC1 ; Cc1cc(C2CC2)cc(-c2c(-c3ccc4c(=O)[nH]nc(CNC(=O)OC(C)(C)C)c4c3)cnn2C)c1C#N ; Cc1cc(C2CC2)cc(-c2c(-c3ccc4c(=O)[nH]nc(CN)c4c3)cnn2C)c1C#N]

### Example 12-14

Step 1: A mixture of Intermediate J (350 mg, 0.872 mmol, 1.00 eq.), 2-(4-bromo-2-methyl-pyrazol-3-yl)-4-chloro-6-methyl-benzonitrile, intermediate D-19 (271 mg, 0.872 mmol, 1.00 eq.), sodium bicarbonate (220 mg, 2.62 mmol, 3.00 eq.) and di-tert-butyl(cyclopentyl)phosphane;dichloropalladium;iron (57 mg, 0.087 mmol, 0.10 eq.) in dioxane (3 mL) and water (0.6 mL) was degassed and purged with nitrogen 3 times and stirred at 80° C. for 1 hour. The mixture was then concentrated and the residue purified by prep-TLC (SiO₂, dichloromethane:methyl alcohol=10:1) to give tert-butyl N-[[7-[5-(5-chloro-2-cyano-3-methyl-phenyl)-1-methyl-pyrazol-4-yl]-4-oxo-3H-phthalazin-1-yl]methyl]carbamate (135 mg, 0.174 mmol, 20% yield) as a yellow solid. LCMS [M+1]⁺=505.2.

Step 2: A mixture of tert-butyl N-[[7-[5-(5-chloro-2-cyano-3-methyl-phenyl)-1-methyl-pyrazol-4-yl]-4-oxo-3H-phthalazin-1-yl]methyl]carbamate (35 mg, 0.069 mmol, 1.00 eq.), cyclopropylboronic acid (24 mg, 0.277 mmol, 4.00 eq.), tricyclohexyl-phosphine (2 mg, 0.007 mmol, 2.3 μL, 0.10 eq.), Pd₂(dba)₃ (6.4 mg, 0.007 mmol, 0.10 eq.) and potassium phosphate (44 mg, 0.208 mmol, 3.00 eq.) in dioxane (2 mL) was degassed and purged with nitrogen 3 times, and stirred at 100° C. for 12 hours. The mixture was then concentrated and the residue purified by prep-TLC (SiO₂, dichloromethane:methyl alcohol=10:1) to give tert-butyl N-[[7-[5-(2-cyano-5-cyclopropyl-3-methyl-phenyl)-1-methyl-pyrazol-4-yl]-4-oxo-3H-phthalazin-1-yl]methyl]carbamate (23 mg, 0.038 mmol, 54% yield) as a yellow solid. LCMS [M+1]⁺=511.3.

Step 3: A mixture of tert-butyl N-[[7-[5-(2-cyano-5-cyclopropyl-3-methyl-phenyl)-1-methyl-pyrazol-4-yl]-4-oxo-3H-phthalazin-1-yl]methyl]carbamate (18 mg, 0.035 mmol, 1.00 eq.) in trifluoroacetic acid (0.5 mL) and dichloromethane (1.5 mL) was purged with nitrogen 3 times, and stirred at 15° C. for 1 hour. The mixture was then concentrated and the residue purified by prep-HPLC (Phenomenex luna C18 150×25 mm 10 μm; mobile phase: [water (0.1% trifluoroacetic acid)-ACN]; B %: 15%-45%, 10 min) to give 2-[4-[4-(aminomethyl)-1-oxo-2H-phthalazin-6-yl]-2-methyl-pyrazol-3-yl]-4-cyclopropyl-6-methyl-benzonitrile (11 mg, 0.021 mmol, 61% yield) as a white solid. LCMS [M+1]⁺=411.2; ¹H NMR (400 MHz, DMSO-d₆) δ=12.88 (s, 1H), 8.37 (br s, 3H), 8.28 (s, 1H), 8.10 (d, J=8.4 Hz, 1H), 7.73 (d, J=1.6 Hz, 1H), 7.46 (dd, J=1.6, 8.4 Hz, 1H), 7.37 (s, 1H), 7.28 (d, J=1.6 Hz, 1H), 4.29 (br s, 2H), 3.71 (s, 3H), 3.34-3.34 (m, 3H), 2.10-1.98 (m, 1H), 1.10 (dd, J=2.8, 8.4 Hz, 2H), 0.95-0.80 (m, 2H); ¹H NMR (400 MHz, CD₃OD) δ=8.20 (d, J=8.4 Hz, 1H), 8.13 (s, 1H), 7.75 (d, J=1.6 Hz, 1H), 7.54-7.47 (m, 1H), 7.33 (s, 1H), 7.22 (d, J=1.6 Hz, 1H), 4.41 (s, 2H), 3.79 (s, 3H), 2.53 (s, 3H), 2.09-2.01 (m, 1H), 1.18-1.11 (m, 2H), 0.92-0.79 (m, 2H).

### Example 12-15

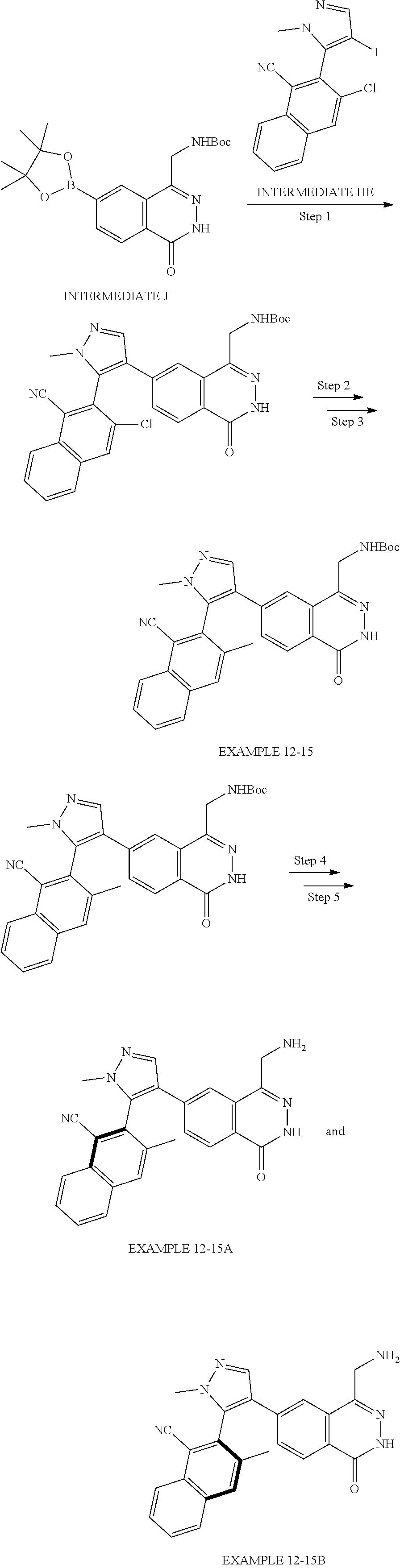

Steps 1-3: Example 12-15, 2-[4-[4-(aminomethyl)-1-oxo-2H-phthalazin-6-yl]-2-methyl-pyrazol-3-yl]-3-methyl-naphthalene-1-carbonitrile was prepared as a yellow gum starting from Intermediate HE and Intermediate J according to the method described for Example 12-9 (2.6 mg, 0.006 mmol, 3% yield over 3 steps). LCMS [M+H]⁺=421.2; ¹H NMR (400 MHz, DMSO-d₆) δ=12.80 (s, 1H), 8.43 (d, J=8.0 Hz, 2H), 8.19-8.15 (m, 1H), 8.13-8.10 (m, 1H), 8.00 (d, J=8.4 Hz, 1H), 7.86 (s, 1H), 7.85-7.79 (m, 2H), 7.26 (d, J=8.4 Hz, 1H), 4.36-4.18 (m, 2H), 4.09 (s, 2H), 3.66 (s, 3H), 2.21 (s, 3H).

Step 4: tert-butyl ((7-(5-(1-cyano-3-methylnaphthalen-2-yl)-1-methyl-1H-pyrazol-4-yl)-4-oxo-3,4-dihydrophthalazin-1-yl)methyl)carbamate (40 mg, 75 μmol) was purified by prep-SFC (Method: column: DAICEL CHIRALPAK AS (250 mm×30 mm, 10 μm); mobile phase: [0.1% ammonium hydroxide/methanol]; B %: 35%-35%, 2.9 mL/min; 55 min) to afford tert-butyl N-[[7-[(1R)-5-(1-cyano-3-methyl-2-naphthyl)-1-methyl-pyrazol-4-yl]-4-oxo-3H-phthalazin-1-yl]methyl]carbamate (14 mg, 26.9 μmol, 35% yield) as yellow solid; LCMS [ESI, M+1]⁺: 521 and tert-butyl N-[[7-[(1R)-5-(1-cyano-3-methyl-2-naphthyl)-1-methyl-pyrazol-4-yl]-4-oxo-3H-phthalazin-1-yl]methyl]carbamate (14 mg, 26.9 μmol, 35% yield) as yellow solid; LCMS [ESI, M+1]⁺: 521.

Step 5: EXAMPLE 12-15A. To a solution of tert-butyl N-[[7-[(1R)-5-(1-cyano-3-methyl-2-naphthyl)-1-methyl-pyrazol-4-yl]-4-oxo-3H-phthalazin-1-yl]methyl]carbamate (14 mg, 26.9 μmol, 1.00 eq.) in methanol (1.0 mL) was added hydrochloride/methanol (4.00 M, 1.0 mL). The mixture was stirred at 20° C. for 5 hours. The mixture was concentrated in vacuo and the residue was diluted with methanol (2 mL) and water (15 mL). The resulting mixture was then dried via lyophilization to afford 2-[(2R)-4-[4-(aminomethyl)-1-oxo-2H-phthalazin-6-yl]-2-methyl-pyrazol-3-yl]-3-methyl-naphthalene-1-carbonitrile (15.6 mg) as yellow solid. LCMS [ESI, M+1]+: 421.2. ¹H NMR (400 MHz, MeOD-d₄) δ=8.34 (d, J=7.2 Hz, 2H), 8.20-8.15 (m, 1H), 8.14-8.08 (m, 2H), 7.81-7.75 (m, 2H), 7.73 (s, 1H), 7.50-7.45 (m, 1H), 4.40-4.19 (m, 2H), 3.75 (s, 3H), 2.29 (s, 3H). Same method was used to prepare EXAMPLE 12-15A from tert-butyl N-[[7-[(1S)-5-(1-cyano-3-methyl-2-naphthyl)-1-methyl-pyrazol-4-yl]-4-oxo-3H-phthalazin-1-yl]methyl]carbamate as a yellow solid (13.6 mg). LCMS [ESI, M+1]⁺: 421.2, ¹H NMR (400 MHz, MeOD-d₄) δ=8.37-8.30 (m, 2H), 8.21-8.15 (m, 1H), 8.14-8.09 (m, 2H), 7.82-7.76 (m, 2H), 7.73 (s, 1H), 7.50-7.45 (m, 1H), 4.42-4.19 (m, 2H), 3.75 (s, 3H), 2.29 (s, 3H).

### Example 12-16

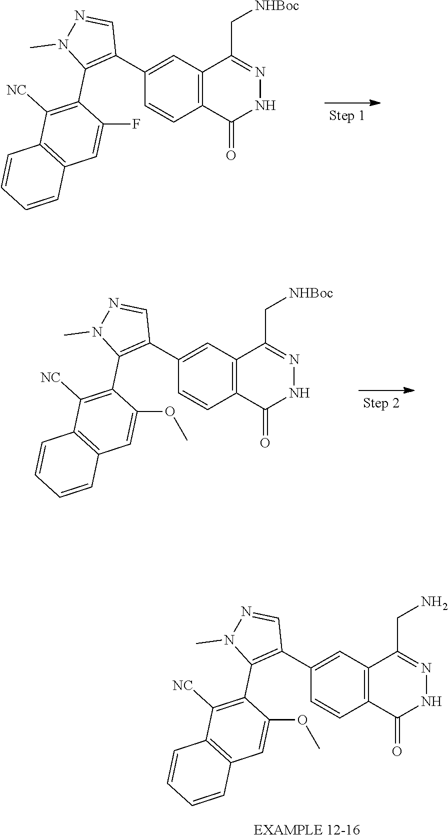 [SMILES: Cn1ncc(-c2ccc3c(=O)[nH]nc(CNC(=O)OC(C)(C)C)c3c2)c1-c1c(F)cc2ccccc2c1C#N ; COc1cc2ccccc2c(C#N)c1-c1c(-c2ccc3c(=O)[nH]nc(CNC(=O)OC(C)(C)C)c3c2)cnn1C ; COc1cc2ccccc2c(C#N)c1-c1c(-c2ccc3c(=O)[nH]nc(CN)c3c2)cnn1C]

Step 1: To a solution of tert-butyl N-[[7-[5-(1-cyano-3-fluoro-2-naphthyl)-1-methyl-pyrazol-4-yl]-4-oxo-3H-phthalazin-1-yl]methyl]carbamate (100 mg, 0.19 mmol, 1.00 eq.) in methyl alcohol (3 mL) was added sodium methoxide (309 mg, 5.72 mmol, 30.0 eq.) and the mixture was stirred at 110° C. for 5 hours. After such time the mixture was diluted with ethyl acetate (20 mL) and washed with brine (20 mL×3), dried over anhydrous sodium sulfate, filtered, concentrated and the residue was purified by prep-TLC (SiO₂, dichloromethane:methyl alcohol=5%) to give tert-butyl N-[[7-[5-(1-cyano-3-methoxy-2-naphthyl)-1-methyl-pyrazol-4-yl]-4-oxo-3H-phthalazin-1-yl]methyl]carbamate (30 mg, 0.056 mmol, 29% yield) as a yellow solid. LCMS [M+1]⁺=537.2.

Step 2: To a solution of tert-butyl N-[[7-[5-(1-cyano-3-methoxy-2-naphthyl)-1-methyl-pyrazol-4-yl]-4-oxo-3H-phthalazin-1-yl]methyl]carbamate (30 mg, 0.056 mmol, 1.00 eq.) in dichloromethane (1.5 mL) was added trifluoroacetic acid (0.5 mL) and the mixture was stirred at 25° C. for 0.5 hour. The mixture was then concentrated and the residue was purified by prep-HPLC, purification method 4-6 to give 2-[4-[4-(aminomethyl)-1-oxo-2H-phthalazin-6-yl]-2-methyl-pyrazol-3-yl]-3-methoxy-naphthalene-1-carbonitrile (8.33 mg, 0.017 mmol, 31% yield, HCl) as a yellow solid. LCMS [M+1]⁺=437.2; ¹H NMR (400 MHz, DMSO-d₆) δ=12.81 (s, 1H), 8.51 (br s, 3H), 8.36 (s, 1H), 8.23-8.10 (m, 2H), 8.00 (br t, J=8.0 Hz, 2H), 7.85 (s, 1H), 7.81-7.61 (m, 2H), 7.33 (br d, J=8.4 Hz, 1H), 4.41-4.14 (m, 2H), 3.97 (s, 3H), 3.68 (s, 3H).

### Example 12-17

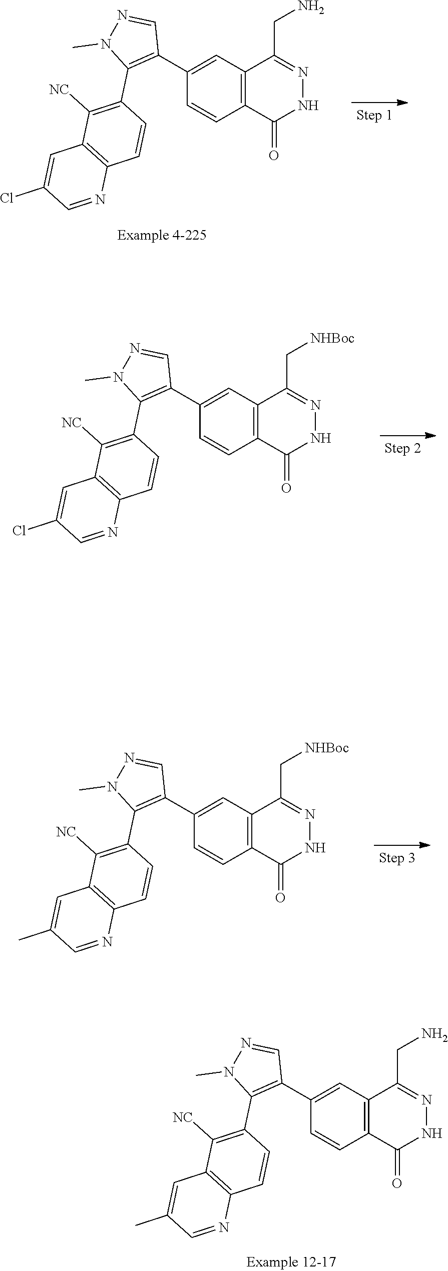 [SMILES: Cn1ncc(-c2ccc3c(=O)[nH]nc(CN)c3c2)c1-c1ccc2ncc(Cl)cc2c1C#N ; Cn1ncc(-c2ccc3c(=O)[nH]nc(CNC(=O)OC(C)(C)C)c3c2)c1-c1ccc2ncc(Cl)cc2c1C#N ; Cc1cnc2ccc(-c3c(-c4ccc5c(=O)[nH]nc(CNC(=O)OC(C)(C)C)c5c4)cnn3C)c(C#N)c2c1 ; Cc1cnc2ccc(-c3c(-c4ccc5c(=O)[nH]nc(CN)c5c4)cnn3C)c(C#N)c2c1]

Step 1: To a solution of Example 4-225, 6-[4-[4-(aminomethyl)-1-oxo-2H-phthalazin-6-yl]-2-methyl-pyrazol-3-yl]-3-chloro-quinoline-5-carbonitrile (150 mg, 0.34 mmol, 1.00 eq.) in dichloromethane (10 mL) was added diisopropylethylamine (1.02 mmol, 0.18 mL, 3.00 eq.) and di-tert-butyl dicarbonate (111 mg, 0.51 mmol, 1.50 eq.) and the mixture was stirred at 25° C. for 2 hours. The reaction mixture was then diluted with water (50 mL), extracted with ethyl acetate (50 mL×3) and the combined organic layers were washed with brine (30 mL), dried over sodium sulfate, filtered, concentrated and the residue was purified by prep-TLC (SiO₂, petroleum ether:ethyl acetate 50%) to give tert-butyl N-[[7-[5-(3-chloro-5-cyano-6-quinolyl)-1-methyl-pyrazol-4-yl]-4-oxo-3H-phthalazin-1-yl] methyl]carbamate (130 mg, 0.24 mmol, 71% yield) as a yellow solid. LCMS [M+1]⁺=542.1.

Step 2: To a solution of tert-butyl N-[[7-[5-(3-chloro-5-cyano-6-quinolyl)-1-methyl-pyrazol-4-yl]-4-oxo-3H-phthalazin-1-yl]methyl]carbamate (120 mg, 0.22 mmol, 1.00 eq.) in dioxane (5 mL) was added potassium carbonate (92 mg, 0.66 mmol, 3.00 eq.), di-tert-butyl(cyclopentyl)phosphane;dichloropalladium-iron (14 mg, 0.022 mmol, 0.10 eq.) and methylboronic acid (66 mg, 1.11 mmol, 5.00 eq.). The mixture was stirred at 100° C. for 2 hours and then the reaction mixture was diluted with water (50 mL) and extracted with ethyl acetate (50 mL×3). The combined organic layers were washed with brine (30 mL), dried over sodium sulfate, filtered, concentrated and the residue purified by prep-TLC (SiO₂, ethyl acetate) to give tert-butyl N-[[7-[5-(5-cyano-3-methyl-6-quinolyl)-1-methyl-pyrazol-4-yl]-4-oxo-3H-phthalazin-1-yl]methyl]carbamate (56 mg, 0.107 mmol, 49% yield) as a yellow solid. LCMS [M+1]522.3.

Step 3: To a solution of tert-butyl N-[[7-[5-(5-cyano-3-methyl-6-quinolyl)-1-methyl-pyrazol-4-yl]-4-oxo-3H-phthalazin-1-yl]methyl]carbamate (66 mg, 0.127 mmol, 1.00 eq.) in dichloromethane (1 mL) was added trifluoroacetic acid (0.3 mL) and the mixture was stirred at 25° C. for 0.5 hour. The reaction mixture was then concentrated and the residue purified by prep-HPLC (purification method 4-6) to give 6-[4-[4-(aminomethyl)-1-oxo-2H-phthalazin-6-yl]-2-methyl-pyrazol-3-yl]-3-methyl-quinoline-5-carbonitrile (14 mg, 0.030 mmol, 24% yield, HCl) as a yellow solid. LCMS [M+1]⁺=422.1; ¹H NMR (400 MHz, DMSO-d₆) δ=12.83 (s, 1H), 9.08 (d, J=2.0 Hz, 1H), 8.52 (br d, J=8.0 Hz, 4H), 8.37 (s, 1H), 8.33-8.30 (m, 1H), 8.01 (d, J=5.2 Hz, 1H), 7.99 (d, J=5.2 Hz, 1H), 7.90 (d, J=1.2 Hz, 1H), 7.34 (dd, J=1.6, 8.4 Hz, 1H), 4.33-4.23 (m, 2H), 3.79 (s, 3H), 2.61 (s, 3H).

### Example 12-18

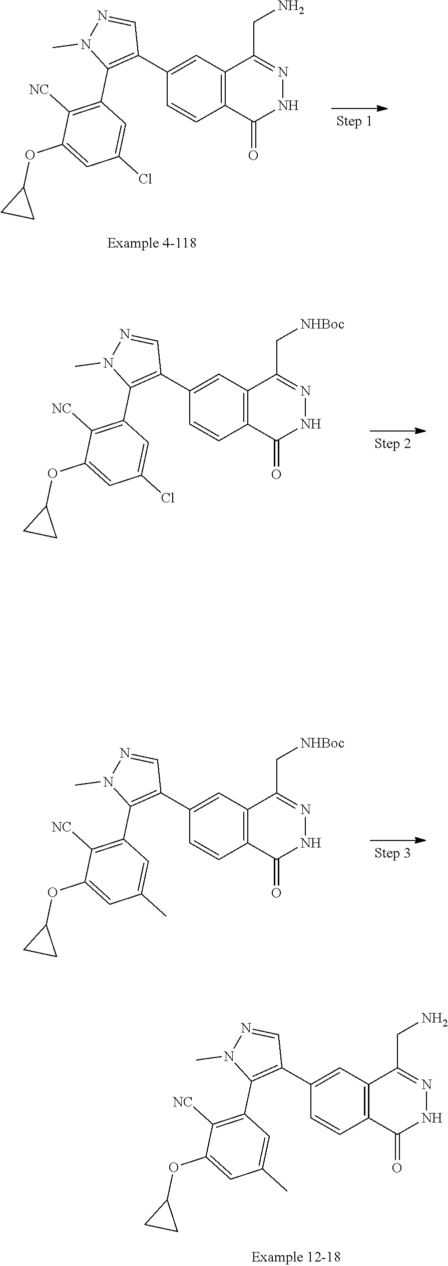 [SMILES: Cn1ncc(-c2ccc3c(=O)[nH]nc(CN)c3c2)c1-c1cc(Cl)cc(OC2CC2)c1C#N ; Cn1ncc(-c2ccc3c(=O)[nH]nc(CNC(=O)OC(C)(C)C)c3c2)c1-c1cc(Cl)cc(OC2CC2)c1C#N ; Cc1cc(OC2CC2)c(C#N)c(-c2c(-c3ccc4c(=O)[nH]nc(CNC(=O)OC(C)(C)C)c4c3)cnn2C)c1 ; Cc1cc(OC2CC2)c(C#N)c(-c2c(-c3ccc4c(=O)[nH]nc(CN)c4c3)cnn2C)c1]

Example 12-18, 2-(4-(4-(aminomethyl)-1-oxo-1,2-dihydrophthalazin-6-yl)-1-methyl-1H-pyrazol-5-yl)-6-cyclopropoxy-4-methylbenzonitrile, was prepared as a white solid starting from Example 4-118 according to the same method as described for Example 12-17 (11 mg, 0.022 mmol, 65% yield, 3% yield over 3 steps). LCMS [M+1]⁺=427.2; ¹H NMR (400 MHz, DMSO-d₆) δ=12.85 (s, 1H), 8.54 (br s, 3H), 8.28 (s, 1H), 8.11 (d, J=8.4 Hz, 1H), 7.70 (d, J=1.2 Hz, 1H), 7.58 (s, 1H), 7.51 (dd, J=1.6, 8.4 Hz, 1H), 7.17 (s, 1H), 4.34-4.10 (m, 3H), 3.71 (s, 3H), 2.50 (br s, 3H), 0.94-0.85 (m, 2H), 0.84-0.71 (m, 2H); ¹H NMR (400 MHz, MeOD) δ=8.20 (d, J=8.4 Hz, 1H), 8.12 (s, 1H), 7.78 (s, 1H), 7.56 (s, 1H), 7.52 (dd, J=1.6, 8.4 Hz, 1H), 7.13 (s, 1H), 4.54-4.35 (m, 2H), 4.05 (tt, J=2.8, 6.0 Hz, 1H), 3.79 (s, 3H), 2.55 (s, 3H), 0.92 (m, 2H), 0.86-0.73 (m, 2H).

### Example 12-19

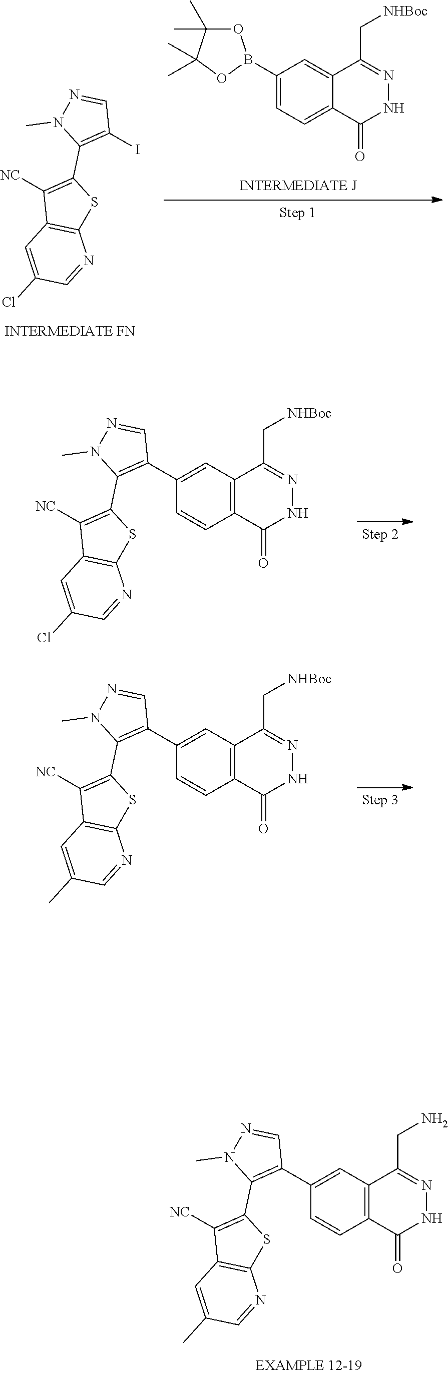 [SMILES: Cn1ncc(I)c1-c1sc2ncc(Cl)cc2c1C#N ; CC(C)(C)OC(=O)NCc1n[nH]c(=O)c2ccc(B3OC(C)(C)C(C)(C)O3)cc12 ; Cn1ncc(-c2ccc3c(=O)[nH]nc(CNC(=O)OC(C)(C)C)c3c2)c1-c1sc2ncc(Cl)cc2c1C#N ; Cc1cnc2sc(-c3c(-c4ccc5c(=O)[nH]nc(CNC(=O)OC(C)(C)C)c5c4)cnn3C)c(C#N)c2c1 ; Cc1cnc2sc(-c3c(-c4ccc5c(=O)[nH]nc(CN)c5c4)cnn3C)c(C#N)c2c1]

Step 1: A mixture of 5-chloro-2-(4-iodo-2-methyl-pyrazol-3-yl)thieno[2,3-b]pyridine-3-carbonitrile (168 mg, 419 μmol, 1.00 eq.), INTERMEDIATE J (219 mg, 545 μmol, 1.30 eq.), Pd(dtbpf)Cl₂ (27.3 mg, 41.9 μmol, 0.10 eq.), sodium bicarbonate (106 mg, 1.26 mmol, 48.9 μL, 3.00 eq.) in dioxane (1.0 mL) and water (0.20 mL) was purged with nitrogen for 3 times and stirred at 80° C. for 1 hour under nitrogen atmosphere. The mixture was concentrated under reduced pressure and the residue was purified by column chromatography (SiO₂, Ethyl acetate/Petroleum ether 10-100%) to give tert-butyl N-[[7-[5-(5-chloro-3-cyano-thieno[2,3-b]pyridin-2-yl)-1-methyl-pyrazol-4-yl]-4-oxo-3H-phthalazin-1-yl]methyl]carbamate (150 mg, 56% yield) as a white solid. LCMS [M+1]⁺=548.1

Step 2: A mixture of tert-butyl N-[[7-[5-(5-chloro-3-cyano-thieno[2,3-b]pyridin-2-yl)-1-methyl-pyrazol-4-yl]-4-oxo-3H-phthalazin-1-yl]methyl]carbamate (100 mg, 182 μmol, 1.00 eq.), methylboronic acid (76.5 mg, 1.28 mmol, 7.00 eq.), Pd(dtbpf)Cl₂ (11.9 mg, 18.3 μmol, 0.10 eq.) and potassium phosphate (116 mg, 547 μmol, 3.00 eq.) in water (0.60 mL) and dioxane (3.0 mL) was degassed and stirred at 100° C. for 2 hours under nitrogen atmosphere. The mixture was concentrated under reduced pressure and the residue was purified by column chromatography (SiO₂, Ethyl acetate/Petroleum ether 10-100%) to give tert-butyl N-[[7-[5-(3-cyano-5-methyl-thieno[2,3-b]pyridin-2-yl)-1-methyl-pyrazol-4-yl]-4-oxo-3H-phthalazin-1-yl]methyl]carbamate (40 mg, 26% yield) as a white solid. LCMS [M+1]⁺=528.2

Step 3: A solution of tert-butyl N-[[7-[5-(3-cyano-5-methyl-thieno[2,3-b]pyridin-2-yl)-1-methyl-pyrazol-4-yl]-4-oxo-3H-phthalazin-1-yl]methyl]carbamate (35.0 mg, 66.3 μmol, 1.00 eq.) in trifluoroacetic acid (1.0 mL) and dichloromethane (3.0 mL) was degassed and stirred at 10° C. for 2 hours under nitrogen atmosphere. The mixture was concentrated under reduced pressure and the residue was purified by prep-HPLC (column: 3_Phenomenex Luna C18 75×30 mm, 3 μm; mobile phase: [water (0.05% HCl)-ACN]; B %: 16%-36%, 6.5 min) to give 2-[4-[4-(aminomethyl)-1-oxo-2H-phthalazin-6-yl]-2-methyl-pyrazol-3-yl]-5-methyl-thieno[2,3-b]pyridine-3-carbonitrile hydrochloride (12 mg, 37% yield) as a white solid. LCMS [M+1]=428.1. ¹H NMR (400 MHz, MeOD-d₄) δ=7.97 (d, J=1.6 Hz, 1H), 7.53 (d, J=8.4 Hz, 1H), 7.51 (d, J=1.2 Hz, 1H), 7.47 (s, 1H), 7.22 (d, J=1.6 Hz, 1H), 6.93 (dd, J=1.6, 8.4 Hz, 1H), 3.77 (s, 2H), 3.31 (s, 3H), 1.87 (s, 3H).

### Example 12-20

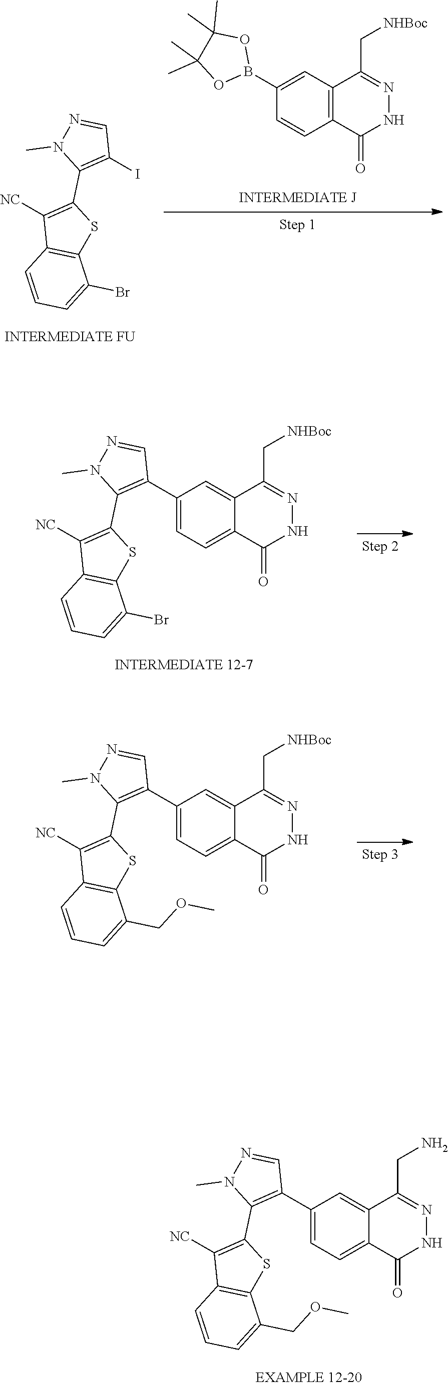 [SMILES: Cn1ncc(I)c1-c1sc2c(Br)cccc2c1C#N ; CC(C)(C)OC(=O)NCc1n[nH]c(=O)c2ccc(B3OC(C)(C)C(C)(C)O3)cc12 ; Cn1ncc(-c2ccc3c(=O)[nH]nc(CNC(=O)OC(C)(C)C)c3c2)c1-c1sc2c(Br)cccc2c1C#N ; COCc1cccc2c(C#N)c(-c3c(-c4ccc5c(=O)[nH]nc(CNC(=O)OC(C)(C)C)c5c4)cnn3C)sc12 ; COCc1cccc2c(C#N)c(-c3c(-c4ccc5c(=O)[nH]nc(CN)c5c4)cnn3C)sc12]

Step 1: A mixture of Intermediate FU (600 mg, 1.35 mmol, 1.00 eq.), Intermediate J (650 mg, 1.62 mmol, 1.20 eq.), Pd(dppf)Cl₂ (99.0 mg, 135 μmol, 0.10 eq.), sodium bicarbonate (227 mg, 2.70 mmol, 105 μL, 2.00 eq.) in water (0.20 mL) and dioxane (2.0 mL) was degassed and stirred at 70° C. for 3 hours under nitrogen atmosphere. After being cooled to room temperature, the mixture was concentrated under vacuum. The residue was diluted with ethyl acetate (30 mL) and water (30 mL). The layers were separated and the aqueous phase was extracted with ethyl acetate (3×20 mL). The organic layer was dried over sodium sulfate, filtered and concentrated under vacuum. The residue was purified by flash silica gel chromatography (Ethyl acetate/Petroleum ether 60-70%) to give a crude product. The crude product was further triturated with Petroleum ether/Ethyl acetate 1:1 (10.0 mL) at 20° C. for 30 mins to give tert-butyl N-[[7-[5-(7-bromo-3-cyano-benzothiophen-2-yl)-1-methyl-pyrazol-4-yl]-4-oxo-3H-phthalazin-1-yl]methyl]carbamate, Intermediate 12-7 (400 mg, 50% yield) as a white solid. ¹H NMR (400 MHz, DMSO-d₆) δ=12.53 (s, 1H), 8.26-8.16 (m, 1H), 8.14-8.06 (m, 2H), 8.05-7.99 (m, 1H), 7.96-7.90 (m, 1H), 7.68-7.59 (m, 1H), 7.54-7.47 (m, 1H), 7.37 (s, 1H), 4.31 (d, J=6.0 Hz, 2H), 3.96 (s, 3H), 1.38 (s, 9H).

Step 2: A mixture of Intermediate 12-7 (50.0 mg, 84.5 μmol, 1.00 eq.), tributyl(methoxymethyl)stannane (141.6 mg, 423 μmol, 5.00 eq.), and tetrakis[triphenylphosphine]palladium(0) (9.77 mg, 8.45 μmol, 0.1 eq.) in N,N-dimethylformamide (2.0 mL) was degassed and stirred at 100° C. for 12 hours under nitrogen atmosphere. Another batch of tetrakis[triphenylphosphine]palladium(0) (20.0 mg, 17.3 μmol, 0.02 eq.) was added and the mixture was stirred at 100° C. for additional 12 hours under nitrogen atmosphere. The reaction mixture was diluted with water (10.0 mL) and extracted with ethyl acetate (10.0 mL×3). The organic layers were washed with brine (10.0 mL), dried over anhydrous sodium sulfate, filtered and concentrated under reduced pressure and the residue was purified by prep-TLC (SiO₂, Petroleum ether/Ethyl acetate 1:2) to give tert-butyl N-[[7-[5-[3-cyano-7-(methoxymethyl)benzothiophen-2-yl]-1-methyl-pyrazol-4-yl]-4-oxo-3H-phthalazin-1-yl]methyl]carbamate (30 mg, 64% yield) as a white solid. LCMS [M+1]⁺=557.2. ¹H NMR (400 MHz, CDCl₃-d) δ=10.20 (s, 1H), 8.23 (d, J=8.4 Hz, 1H), 7.98-7.94 (m, 1H), 7.68-7.66 (m, 3H), 7.56-7.54 (m, 3H), 4.79 (s, 2H), 3.98 (s, 3H), 3.43 (s, 3H), 2.04 (s, 2H), 1.47 (s, 9H).

Step 3: To a solution of tert-butyl N-[[7-[5-[3-cyano-7-(methoxymethyl)benzothiophen-2-yl]-1-methyl-pyrazol-4-yl]-4-oxo-3H-phthalazin-1-yl]methyl]carbamate (30.0 mg, 53.9 μmol, 1.00 eq.) in dichloromethane (0.50 mL) was added trifluoroacetic acid (61.5 mg, 539 μmol, 39.9 μL, 10.0 eq.). The mixture was stirred at 20° C. for 4 hours. The mixture was concentrated to get the crude product which was purified by pre-HPLC (column: Welch Xtimate C18 150×25 mm×5 μm; mobile phase: [water (0.05% HCl)-ACN]; B %: 7%-37%, 10 min to give 2-[4-[4-(aminomethyl)-1-oxo-2H-phthalazin-6-yl]-2-methyl-pyrazol-3-yl]-7-(methoxymethyl) benzothiophene-3-carbonitrile hydrochloride (8 mg, 28% yield) as a white solid. LCMS [M+1]⁺=457.2. ¹H NMR (400 MHz, DMSO-d₆) δ=12.89 (s, 1H), 8.45 (s, 3H), 8.36 (s, 1H), 8.12 (dd, J=8.4 Hz, 1H), 7.96 (s, 1H), 7.95 (d, J=8.0 Hz, 1H), 7.68 (d, J=7.6 Hz, 1H), 7.61 (d, J=8.0 Hz, 1H), 7.59-7.52 (m, 1H), 4.81 (s, 2H), 4.38 (q, J=6.0 Hz, 2H), 3.93 (s, 3H), 3.32 (s, 3H).

### Example 12-21

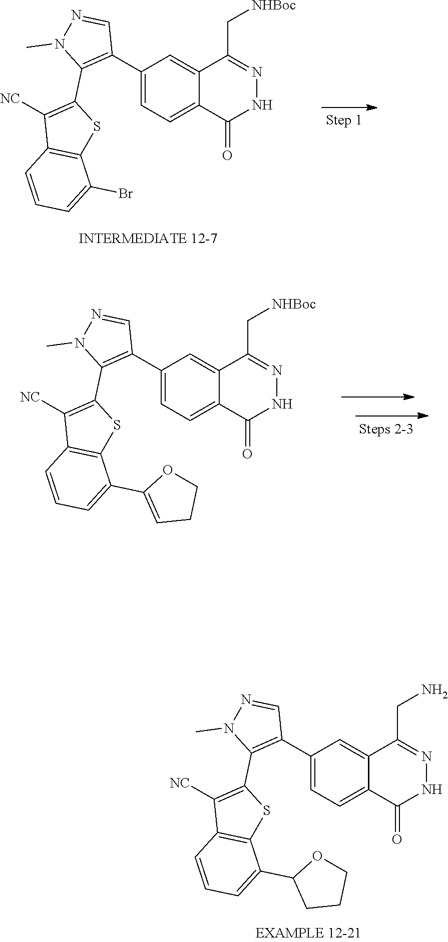 [SMILES: Cn1ncc(-c2ccc3c(=O)[nH]nc(CNC(=O)OC(C)(C)C)c3c2)c1-c1sc2c(Br)cccc2c1C#N ; Cn1ncc(-c2ccc3c(=O)[nH]nc(CNC(=O)OC(C)(C)C)c3c2)c1-c1sc2c(C3=CCCO3)cccc2c1C#N ; Cn1ncc(-c2ccc3c(=O)[nH]nc(CN)c3c2)c1-c1sc2c(C3CCCO3)cccc2c1C#N]

Step 1: A mixture of Intermediate 12-7 (100 mg, 169 μmol, 1.00 eq.), tributyl(2,3-dihydrofuran-5-yl)stannane (100 mg, 278 μmol, 1.65 eq.), tetrakis[triphenylphosphine]palladium(0) (20.0 mg, 17.3 μmol, 1.02e-1 eq.) in dioxane (5.0 mL) was degassed and stirred at 100° C. for 12 hours under nitrogen atmosphere. After being cooled to room temperature, the mixture was concentrated under vacuum. The residue was quenched with saturated potassium fluoride (20 mL). The mixture was extracted with ethyl acetate (3×30 mL). The combined organic extracts were washed with saturated brine (20 mL) and then dried over sodium sulfate. The residue was purified by prep-TLC (SiO₂, Dichloromethane/Methanol 10:1) to give tert-butyl N-[[7-[5-[3-cyano-7-(2,3-dihydrofuran-5-yl)benzothiophen-2-yl]-1-methyl-pyrazol-4-yl]-4-oxo-3H-phthalazin-1-yl]methyl]carbamate (130 mg, 150 μmol, 89% yield, 67% purity) as a white solid. ¹H NMR (400 MHz, CDCl₃-d) δ=9.94 (s, 1H), 8.22 (d, J=8.0 Hz, 1H), 7.94 (d, J=8.0 Hz, 1H), 7.79 (d, J=7.6 Hz, 1H), 7.68-7.64 (m, 2H), 7.58 (t, J=7.6 Hz, 1H), 7.49-7.43 (m, 3H), 5.60 (t, J=2.8 Hz, 1H), 5.29-5.28 (m, 1H), 4.57-4.55 (m, 2H), 3.98 (s, 3H), 2.91 (dt, J=2.9, 9.5 Hz, 2H), 1.48 (s, 9H).

Step 2: To a solution of tert-butyl N-[[7-[5-[3-cyano-7-(2,3-dihydrofuran-5-yl)benzothiophen-2-yl]-1-methyl-pyrazol-4-yl]-4-oxo-3H-phthalazin-1-yl]methyl]carbamate (80.0 mg, 138 μmol, 1.00 eq.) in THF (5.0 mL) was added acetic acid (16.6 mg, 276 μmol, 15.8 μL, 2.00 eq.) and palladium on carbon (80.0 mg, 10%% purity, 1.00 eq.). The mixture was stirred at 40° C. for 12 hours under H₂ atmosphere (15 psi). The resulting mixture was filtered over through a pad of celite, and the filtrate was concentrated under reduced pressure to give tert-butyl N-[[7-[5-(3-cyano-7-THF-2-yl-benzothiophen-2-yl)-1-methyl-pyrazol-4-yl]-4-oxo-3H-phthalazin-1-yl]methyl]carbamate (17 mg, 13% yield) as a yellow solid. LCMS [M+1]⁺=583.7.

Step 3: To a solution of tert-butyl N-[[7-[5-(3-cyano-7-THF-2-yl-benzothiophen-2-yl)-1-methyl-pyrazol-4-yl]-4-oxo-3H-phthalazin-1-yl]methyl]carbamate (17.0 mg, 29.2 μmol, 1.00 eq.) in dichloromethane (3.0 mL) was added trifluoroacetic acid (924 mg, 8.10 mmol, 0.6 mL, 278 eq.). The mixture was stirred at 20° C. for 1 hour before being concentrated in vacuum. The residue was purified by prep-HPLC (column: Welch Xtimate C18 150×25 mm×5 μm; mobile phase: [water (0.05% HCl)-ACN];B %: 10%-40%, 10 min) to give 2-[4-[4-(aminomethyl)-1-oxo-2H-phthalazin-6-yl]-2-methyl-pyrazol-3-yl]-7-THF-2-yl-benzothiophene-3-carbonitrile hydrochloride (6 mg, 38% yield) as a light pink solid. LCMS [M+1]⁺=483.2. ¹H NMR (400 MHz, MeOD-d₄) δ=8.24 (d, J=8.4 Hz, 1H), 8.16 (s, 1H), 7.91-7.85 (m, 2H), 7.67-7.60 (m, 2H), 7.53 (d, J=7.6 Hz, 1H), 5.27 (t, J=7.2 Hz, 1H), 4.47-4.34 (m, 2H), 4.21-4.09 (m, 1H), 4.03-3.94 (m, 4H), 2.54-2.43 (m, 1H), 2.19-2.01 (m, 2H), 1.99-1.87 (m, 1H).

### Example 13-1

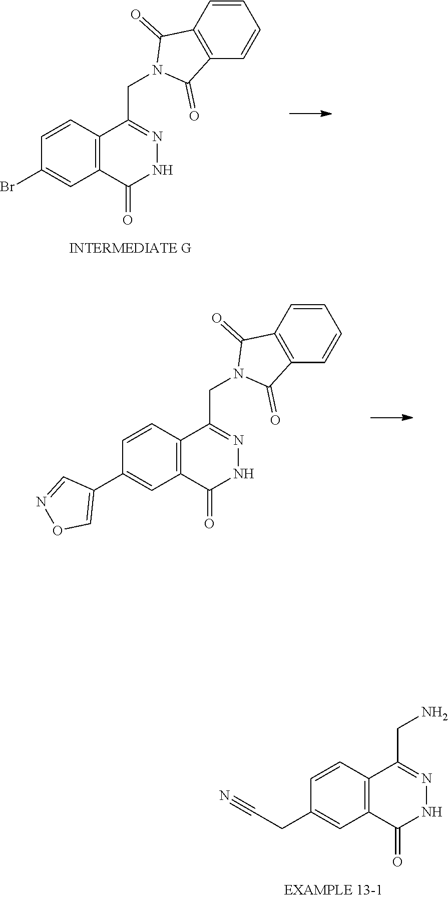 [SMILES: O=C1c2ccccc2C(=O)N1Cc1n[nH]c(=O)c2cc(Br)ccc12 ; O=C1c2ccccc2C(=O)N1Cc1n[nH]c(=O)c2cc(-c3cnoc3)ccc12 ; N#CCc1ccc2c(CN)n[nH]c(=O)c2c1]

Step 1: A mixture of Intermediate G (1.00 g, 2.60 mmol, 1.00 eq.), 4-(4,4,5,5-tetramethyl-1,3,2-dioxaborolan-2-yl)isoxazole (609 mg, 3.12 mmol, 1.20 eq.), potassium fluoride (1.00 M, 7.81 mL, 3.00 eq.) and Pd(dppf)Cl₂ (170 mg, 0.26 mmol, 0.10 eq.) in DMSO (30 mL) was degassed and purged with nitrogen 3 times then stirred at 130° C. for 16 hours. After such time the reaction mixture was diluted with water (100 mL) and extracted with ethyl acetate (30 mL×3). The combined organic extracts were washed with brine (80 mL), dried over anhydrous sodium sulfate, filtered and concentrated under reduced pressure. The residue was purified by column chromatography (SiO₂, petroleum ether/ethyl acetate 1/1 to 1/0, then methyl alcohol/ethyl acetate 1/10 followed by dichloromethane/methyl alcohol 10/1) to give 2-[(6-isoxazol-4-yl-4-oxo-3H-phthalazin-1-yl)methyl]isoindoline-1,3-dione (239 mg, 0.642 mmol, 25% yield) as a red solid. LCMS [M+1]⁺=373.1.

Step 2: To a solution of 2-[(6-isoxazol-4-yl-4-oxo-3H-phthalazin-1-yl)methyl]isoindoline-1,3-dione (200 mg, 0.537 mmol, 1.00 eq.) in ethyl alcohol (10 mL) was added hydrazine hydrate (0.053 mL, 1.07 mmol, 2.00 eq.) and the mixture stirred at 80° C. for 1 hour. After such time the pH of the cooled reaction mixture was adjusted to pH 1 with hydrochloric acid (1.00 M, 2.0 mL) and concentrated under reduced pressure. The residue diluted with hydrochloric acid (1.00 M, 40 mL) and extracted with ethyl acetate (30 mL×3) and the aqueous phase was concentrated under reduced pressure. The residue was purified by prep-HPLC (Waters Xbridge 150-25 mm×5 μm; mobile phase: [water (0.05% ammonia hydroxide v/v)-ACN]; B %: 0/6-20%, 10 min) to give 2-[1-(aminomethyl)-4-oxo-3H-phthalazin-6-yl]acetonitrile, example 13-1 (32 mg, 0.145 mmol, 27% yield) as a white solid. LCMS [M+1]=215.2; ¹H NMR (400 MHz, DMSO-dc) δ=8.27 (d, J=1.2 Hz, 1H), 8.12 (d, J=8.4 Hz, 1H), 7.87 (dd, J=2.0, 8.4 Hz, 1H), 4.26 (s, 2H), 4.03 (s, 2H).

### Example 13-2

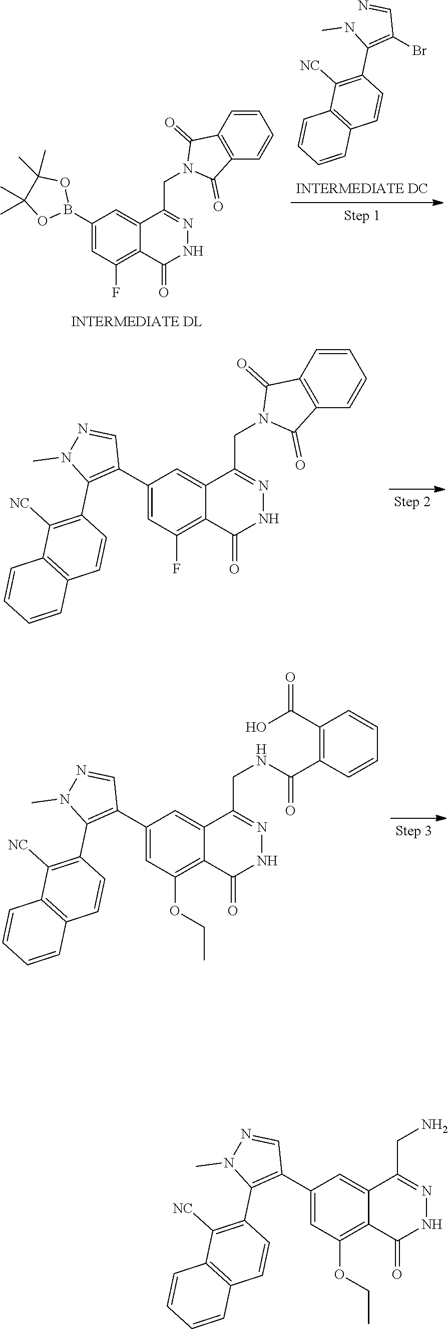

Step 1: To a mixture of 2-[[5-fluoro-4-oxo-7-(4,4,5,5-tetramethyl-1,3,2-dioxaborolan-2-yl)-3H-phthalazin-1-yl]methyl]isoindoline-1,3-dione, Intermediate DL (100 mg, 0.223 mmol, 1.00 eq.), 2-(4-bromo-2-methyl-pyrazol-3-yl)naphthalene-1-carbonitrile, Intermediate DC (70 mg, 0.223 mmol, 1.00 eq.) and sodium bicarbonate (56 mg, 0.668 mmol, 26.0 μL, 3.00 eq) in dioxane (4 mL) and water (0.8 mL) was added di-tert-butyl(cyclopentyl)phosphane;dichloropalladium-iron (14.5 mg, 0.022 mmol, 0.10 eq.) and the reaction mixture was degassed and purged with nitrogen and stirred at 80° C. for 1 hour. After such time the reaction mixture was concentrated under reduced pressure and the residue was purified by prep-TLC (SiO₂, dichloromethane:methanol=10:1) to give 2-[4-[4-[(1,3-dioxoisoindolin-2-yl)methyl]-8-fluoro-1-oxo-2H-phthalazin-6-yl]-2-methyl-pyrazol-3-yl]naphthalene-1-carbonitrile (50 mg, 0.074 mmol, 34% yield) as a white solid. LCMS [M+1]=555.2.

Step 2: To a mixture of 2-[4-[4-[(1,3-dioxoisoindolin-2-yl)methyl]-8-fluoro-1-oxo-2H-phthalazin-6-yl]-2-methyl-pyrazol-3-yl]naphthalene-1-carbonitrile (30 mg, 0.054 mmol, 1.00 eq.) in ethyl alcohol (2 mL) was added sodium ethoxide (74 mg, 3.20 mmol, 76 μL, 59 eq.) in one portion at 20° C. under nitrogen atmosphere. The mixture was stirred at 110° C. for 2 hours in a sealed tube then quenched with concentrated HCl (0.1 mL) to pH=5. The mixture was then concentrated under reduce pressure to give 2-[[7-[5-(1-cyano-2-naphthyl)-1-methyl-pyrazol-4-yl]-5-ethoxy-4-oxo-3H-phthalazin-1-yl]methylcarbamoyl]benzoic acid (20.0 mg, crude) as a white solid which used for the next step without further purification. LCMS [M+1]⁺=599.1.

Step 3: To a mixture of 2-[[7-[5-(1-cyano-2-naphthyl)-1-methyl-pyrazol-4-yl]-5-ethoxy-4-oxo-3H-phthalazin-1-yl]methylcarbamoyl]benzoic acid (20 mg, crude) in ethyl alcohol (4 mL) was added hydrazine hydrate (334 μmol, 16 μL, 10.0 eq.) in one portion at 20° C. under nitrogen. Then the mixture was stirred at 70° C. for 12 hours and after such time the solvent was removed under reduce pressure and the residue dissolved in dimethylsulfoxide (2 mL) to which concentrated HCl (0.2 mL) was added to the reaction mixture to pH 6, and the resulting mixture was purified by Prep-HPLC (Phenomenex Luna C18 75×30 mm×3 μm; mobile phase: [water (0.05% HCl)-ACN]; B %: 17%-37%, 7 min) to give 2-[4-[4-(aminomethyl)-8-ethoxy-1-oxo-2H-phthalazin-6-yl]-2-methyl-pyrazol-3-yl]naphthalene-1-carbonitrile (6 mg, 12 μmol, 36% yield) as a yellow solid. LCMS [M+1]⁺=451.4; 1 H NMR (400 MHz, CD₃OD) δ=8.49 (d, J=8.4 Hz, 1H), 8.25-8.19 (m, 3H), 7.91-7.82 (m, 2H), 7.80 (d, J=8.4 Hz, 1H), 7.33 (s, 1H), 6.78 (d, J=1.2 Hz, 1H), 4.46-4.19 (m, 2H), 3.86 (s, 3H), 3.68-3.38 (m, 2H), 0.94 (t, J=7.2 Hz, 3H).

### Example 13-3

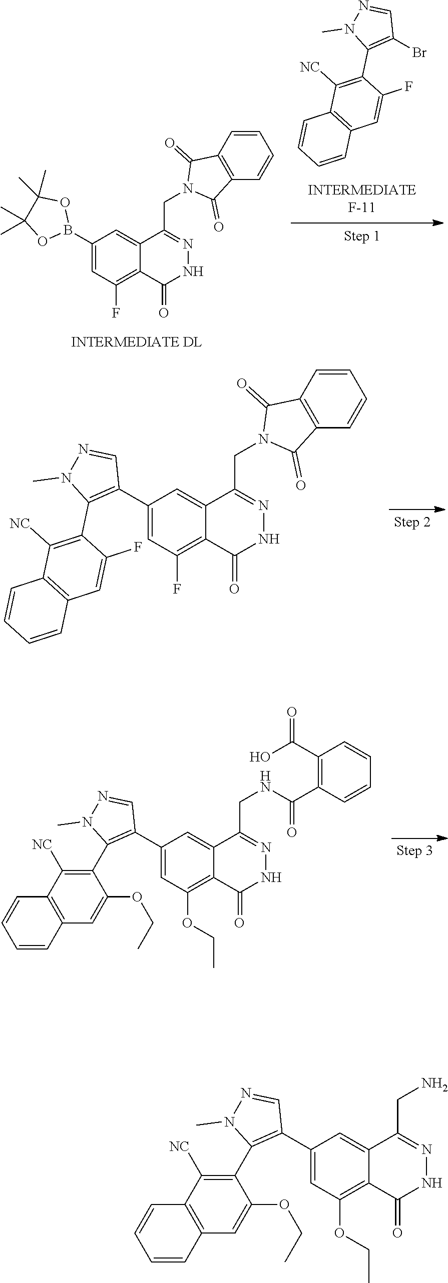

Step 1: To a mixture of 2-[[5-fluoro-4-oxo-7-(4,4,5,5-tetramethyl-1,3,2-dioxaborolan-2-yl)-3H-phthalazin-1-yl]methyl]isoindoline-1,3-dione (61 mg, 0.136 mmol, 1.50 eq), 2-(4-bromo-2-methyl-pyrazol-3-yl)-3-fluoro-naphthalene-1-carbonitrile (30 mg, 0.090 mmol, 1.00 eq) and sodium bicarbonate (23 mg, 0.273 mmol, 3.00 eq) in dioxane (3.0 mL) and H₂O (0.6 mL) was added SPhos Pd G3 (7 mg, 0.009 mmol, 0.1 eq) then stirred at 80° C. for 1 hour under nitrogen atmosphere. The reaction mixture was then concentrated under reduced pressure and purified by prep-TLC (SiO₂, dichloromethane:methanol=10:1) to give 2-[4-[4-[(1,3-dioxoisoindolin-2-yl)methyl]-8-fluoro-1-oxo-2H-phthalazin-6-yl]-2-methyl-pyrazol-3-yl]-3-fluoro-naphthalene-1-carbonitrile (10 mg, 0.005 mmol, 6% yield) as a white solid. LCMS [M+1]⁺=573.3.

Step 2: To a mixture of 2-[4-[4-[(1,3-dioxoisoindolin-2-yl)methyl]-8-fluoro-1-oxo-2H-phthalazin-6-yl]-2-methyl-pyrazol-3-yl]-3-fluoro-naphthalene-1-carbonitrile (10 mg, 0.016 mmol) in ethanol (2.0 mL) was added freshly prepared sodium ethoxide (50 mg in 0.5 mL EtOH) in one portion at 20° C. under N₂ atmosphere. The mixture was stirred at 110° C. for 2 hours in a sealed tube. The reaction was then quenched with concentrated HCl then concentrated under reduce pressure to give 2-[[7-[5-(1-cyano-3-ethoxy-2-naphthyl)-1-methyl-pyrazol-4-yl]-5-ethoxy-4-oxo-3H-phthalazin-1-yl]methylcarbamoyl]benzoic acid (10 mg, crude) as a white solid which used for the next step without further purification. LCMS [M+1]⁺=643.6.

Step 3: To a mixture of 2-[[7-[5-(1-cyano-3-ethoxy-2-naphthyl)-1-methyl-pyrazol-4-yl]-5-ethoxy-4-oxo-3H-phthalazin-1-yl]methylcarbamoyl]benzoic acid (10 mg, 0.016 mmol, 1.00 eq) in ethanol (2.0 mL) was added hydrazine hydrate (0.155 mmol, 8 μL, 10 eq.) in one portion at 20° C. under N₂ atmosphere. The mixture was then stirred at 70° C. for 12 hours, concentrated and the residue dissolved in DMSO (2.0 mL) and the pH adjusted to pH 6 via the addition of concentrated HCl. The resulting mixture was then purified by prep-HPLC (Shim-pack C18 150×25 mm×10 μm; mobile phase: [water (0.1% TFA)-ACN]; B %: 10%-40%, 10 min) to give 2-[4-[4-(aminomethyl)-8-ethoxy-1-oxo-2H-phthalazin-6-yl]-2-methyl-pyrazol-3-yl]-3-ethoxy-naphthalene-1-carbonitrile (3 mg, 0.005 mmol, 31% yield) as a yellow solid. LCMS [M+1]⁺=495.4; ¹H NMR (400 MHz, MeOD) δ=8.25 (s, 1H), 8.10 (t, J=8.0 Hz, 2H), 8.00 (s, 1H), 7.79-7.73 (m, 1H), 7.70-7.63 (m, 1H), 7.37 (d, J=1.2 Hz, 1H), 6.77 (d, J=0.8 Hz, 1H), 4.45-4.27 (m, 4H), 3.82 (s, 3H), 3.68-3.60 (m, 1H), 3.48-3.41 (m, 1H), 1.37 (s, 4H), 0.99 (t, J=6.8 Hz, 3H).

### Example 14-1

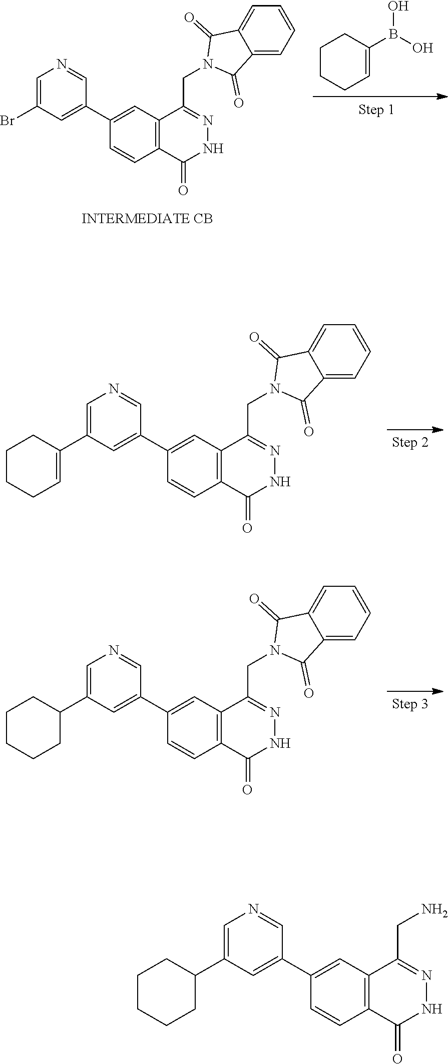

Step 1: A mixture of 2-[[7-(5-bromo-3-pyridyl)-4-oxo-3H-phthalazin-1-yl]methyl]isoindoline-1,3-dione, Intermediate CB (300 mg, 0.650 mmol, 1.00 eq.), cyclohexen-1-ylboronic acid (164 mg, 1.30 mmol, 2.00 eq.), Pd(dppf)Cl₂ (48 mg, 65 μmol, 0.10 eq.), sodium bicarbonate (109 mg, 1.30 mmol, 51 μL, 2.00 eq.) in dioxane (4 mL) and water (0.8 mL) was degassed and purged with nitrogen 3 times and stirred at 80° C. for 2 hours under nitrogen atmosphere. After such time the reaction mixture was concentrated and the residue was purified by column chromatography (SiO₂, petroleum ether:ethyl acetate 5:1 to 0:1) then by prep-HPLC (Phenomenex luna C18 150×25 mm×10 μm; mobile phase: [water (0.1% TFA)-ACN]; B %: 34%-54%, 10 min) to give 2-[[7-[5-(cyclohexen-1-yl)-3-pyridyl]-4-oxo-3H-phthalazin-1-yl]methyl]isoindoline-1,3-dione (20 mg, 7% yield) as a yellow solid. LCMS [M+1]⁺=463.2.

Step 2: To a solution of 2-[[7-[5-(cyclohexen-1-yl)-3-pyridyl]-4-oxo-3H-phthalazin-1-yl]methyl]isoindoline-1,3-dione (10 mg, 22 μmol, 1.00 eq.) in methyl alcohol (2 mL) was added 10% palladium on activated carbon (5 mg, 0.1 eq) and the suspension was degassed and purged with hydrogen 3 times. The mixture was then stirred under hydrogen (15 Psi) at 25° C. for 2 hours. After such time the reaction mixture was filtered and concentrated under reduced pressure to give 2-[[7-(5-cyclohexyl-3-pyridyl)-4-oxo-3H-phthalazin-1-yl]methyl]isoindoline-1,3-dione (10 mg, crude) as a yellow solid which was used into next step directly without further purification. LCMS [M+1]⁺=465.3.

Step 3: To a solution of 2-[[7-(5-cyclohexyl-3-pyridyl)-4-oxo-3H-phthalazin-1-yl]methyl]isoindoline-1,3-dione (10 mg, 22 μmol, 1.00 eq.) in ethyl alcohol (1 mL) was added hydrazine hydrate (11 mg, 0.215 mmol, 11 μL, 10.0 eq.) and the reaction mixture stirred at 80° C. for 0.5 hour. After such time the reaction mixture was concentrated under reduced and purified by prep-HPLC (Phenomenex Gemini-NX C18 75×30 mm×3 μm; mobile phase: [water (0.1% TFA)-ACN]; B %: 15%-25%, 7 min) to give 4-(aminomethyl)-6-(5-cyclohexyl-3-pyridyl)-2H-phthalazin-1-one (3.6 mg, 36% yield) as a yellow solid. LCMS [M+1]⁺=335.2; ¹H NMR (400 MHz, DMSO-dc) δ=12.99 (s, 1H), 8.98 (d, J=2.0 Hz, 1H), 8.60 (d, J=2.0 Hz, 1H), 8.45 (br s, 3H), 8.41-8.37 (m, 1H), 8.33-8.29 (m, 1H), 8.25 (d, J=1.2 Hz, 1H), 8.18 (t, J=2.0 Hz, 1H), 4.63 (br d, J=5.6 Hz, 2H), 2.71-2.65 (m, 1H), 1.92-1.81 (m, 4H), 1.79-1.71 (m, 1H), 1.66-1.51 (m, 2H), 1.48-1.36 (m, 2H), 1.34-1.23 (m, 1H).

### Example 15-1

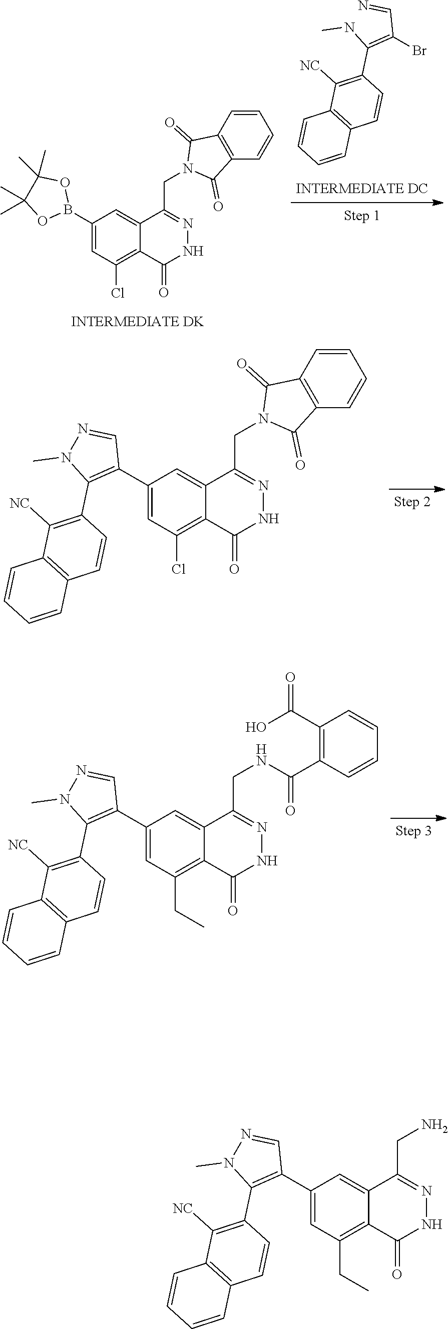

Step 1: A mixture of Intermediate DK (60 mg, 0.129 mmol, 1.00 eq.), 2-(4-bromo-2-methyl-pyrazol-3-yl)naphthalene-1-carbonitrile (40 mg, 0.129 mmol, 1.00 eq.), Pd(dtbpf)Cl₂ (8.4 mg, 0.012 mmol, 0.10 eq.), sodium bicarbonate (33 mg, 0.39 mmol, 3.00 eq.) in dioxane (3.0 mL) and water (0.5 mL) was degassed and purged with nitrogen 3 times, and then stirred at 80° C. for 1 hour under nitrogen atmosphere. The mixture was then concentrated and the residue purified by prep-TLC (SiO₂, dichloromethane:methyl alcohol 10:1) to give 2-[4-[8-chloro-4-[(1,3-dioxoisoindolin-2-yl)methyl]-1-oxo-2H-phthalazin-6-yl]-2-methyl-pyrazol-3-yl]naphthalene-1-carbonitrile (40 mg, 50% yield) as a yellow solid. LCMS [M+1]⁺=571.2.

Step 2: A mixture of 2-[4-[8-chloro-4-[(1,3-dioxoisoindolin-2-yl)methyl]-1-oxo-2H-phthalazin-6-yl]-2-methyl-pyrazol-3-yl]naphthalene-1-carbonitrile (40 mg, 0.070 mmol, 1.00 eq.), triethylborane (69 mg, 0.701 mmol, 0.101 mL, 10.0 eq.), Xantphos (4 mg, 0.007 mmol, 0.10 eq.), potassium carbonate (29 mg, 0.210 mmol, 3.00 eq.) in water (0.6 mL) and THF (3.0 mL) was degassed and purged with nitrogen 3 times, and then the mixture was stirred at 80° C. in a sealed tube for 5 hours. The mixture was then concentrated under reduced pressure and the pH of the residue was adjusted to pH 7 by the addition of hydrochloric acid (1 M) aqueous solution then further concentrated to give 2-[[7-[5-(1-cyano-2-naphthyl)-1-methyl-pyrazol-4-yl]-5-ethyl-4-oxo-3H-phthalazin-1-yl]methylcarbamoyl]benzoic acid (100 mg, crude) as a black solid, which was used into next step directly without further purification. LCMS [M+1]⁺=583.0.

Step 3: A mixture of 2-[[7-[5-(1-cyano-2-naphthyl)-1-methyl-pyrazol-4-yl]-5-ethyl-4-oxo-3H-phthalazin-1-yl]methylcarbamoyl]benzoic acid (100 mg, 0.172 mmol, 1.00 eq.), hydrazine hydrate (3.43 mmol, 0.17 mL, 20.0 eq.) in ethyl alcohol (5 mL) was degassed and purged with nitrogen 3 times, and then the mixture was stirred at 80° C. for 18 hours. The mixture was then concentrated, and the residue acidified with hydrochloric acid (1 M) to pH 6, diluted with ethyl acetate (8 mL) and extracted with water (5 mL×3). The aqueous layers were concentrated under reduced pressure and the residue was purified by prep-HPLC (Phenomenex Luna C18 75×30 mm×3 μm; mobile phase: [water (0.05% hydrochloric acid)-ACN]; B %: 18%-38%, 7 min) to give 2-[4-[4-(aminomethyl)-8-ethyl-1-oxo-2H-phthalazin-6-yl]-2-methyl-pyrazol-3-yl]naphthalene-1-carbonitrile (13 mg, 16% yield) as a yellow solid. LCMS [M+1]435.3; ¹H NMR (400 MHz, DMSO-d₆) δ=12.61 (s, 1H), 8.56 (d, J=8.4 Hz, 1H), 8.48 (br s, 3H), 8.39 (s, 1H), 8.29 (d, J=7.6 Hz, 1H), 8.14 (d, J=8.4 Hz, 1H), 7.93-7.81 (m, 3H), 7.72 (d, J=1.6 Hz, 1H), 7.01 (d, J=1.2 Hz, 1H), 4.38-4.09 (m, 2H), 3.80 (s, 3H), 3.09-2.81 (m, 2H), 0.65 (t, J=7.6 Hz, 3H).

### Example 15-2

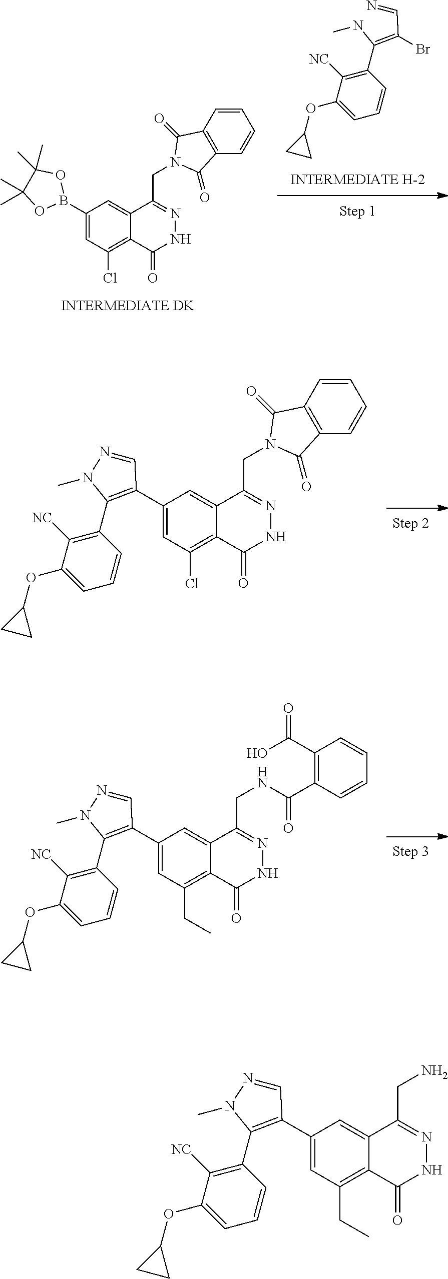

2-(4-(4-(aminomethyl)-8-ethyl-1-oxo-1,2-dihydrophthalazin-6-yl)-1-methyl-1H-pyrazol-5-yl)-6-cyclopropoxybenzonitrile, Example 15-2 was prepared as a white solid (9 mg, 0.019 mmol, 12% yield, HCl salt) following the same 3 step procedure as for example 15-1 starting with Intermediate H-2. LCMS [M+1]⁺ 441.3; ¹H NMR (400 MHz, DMSO-d₆) δ=12.63 (s, 1H), 8.51 (br s, 3H), 8.32 (s, 1H), 7.98-7.88 (m, 1H), 7.75 (d, J=8.4 Hz, 1H), 7.56 (d, J=1.6 Hz, 1H), 7.35 (d, J=7.2 Hz, 1H), 7.17 (d, J=1.2 Hz, 1H), 4.30-4.12 (m, 3H), 3.73 (s, 3H), 3.25-3.01 (m, 2H), 0.94 (t, J=7.2 Hz, 3H), 0.92-0.85 (m, 2H), 0.82-0.67 (m, 2H).

### Example 15-3 and Example 15-4

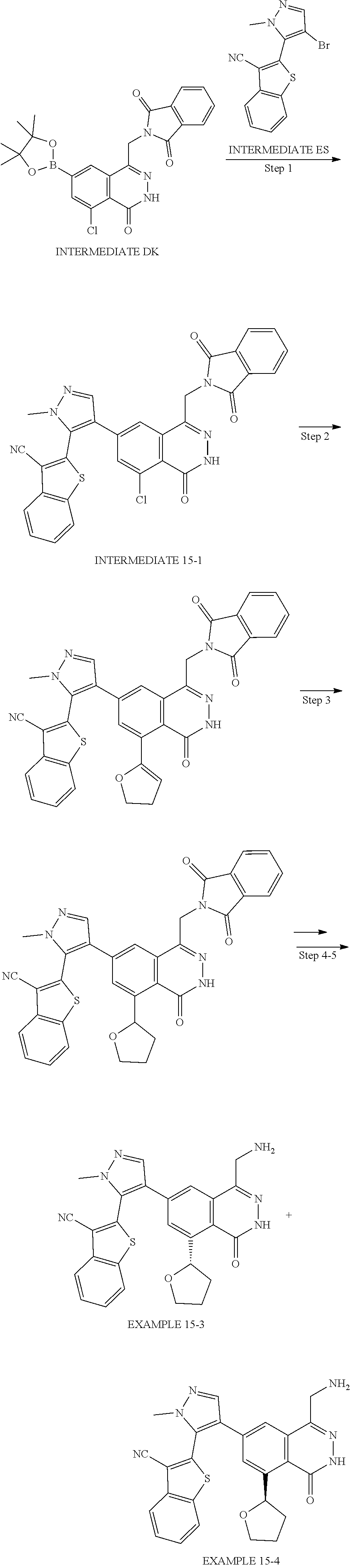

Step 1: To a solution of Intermediate DK (3.07 g, 6.60 mmol, 1.20 eq.) and 2-(4-bromo-1-methyl-1H-pyrazol-5-yl)benzo[b]thiophene-3-carbonitrile (1.75 g, 5.50 mmol, 1.00 eq.) in dimethylformamide (50 mL) was added sodium bicarbonate (924 mg, 11.0 mmol, 428 μL, 2.00 eq.) and Pd(dtbpf)Cl₂ (358 mg, 550 μmol, 0.10 eq.). The mixture was degassed and stirred at 70° C. for 4 hours under nitrogen before being diluted with water (400 mL) and extracted with ethyl acetate (100 mL×3). The organic layer was washed with brine (200 mL×2), dried over anhydrous sodium sulfate, filtered and concentrated in vacuum. The residue was purified by flash silica gel chromatography (Petroleum ether/Ethyl acetate 10-100%) to afford 2-(4-(8-chloro-4-((1,3-dioxoisoindolin-2-yl)methyl)-1-oxo-1,2-dihydrophthalazin-6-yl)-1-methyl-1H-pyrazol-5-yl)benzo[b]thiophene-3-carbonitrile, Intermediate 15-1 (2.20 g, 61% yield) as a light yellow solid. LCMS [M+1]⁺: 577.2. ¹H NMR (400 MHz, DMSO-d₆) δ=12.39 (s, 1H), 8.41 (s, 1H), 8.33-8.26 (m, 1H), 8.07-7.98 (m, 1H), 7.96-7.86 (m, 5H), 7.70-7.61 (m, 2H), 7.57 (d, J=1.6 Hz, 1H), 4.87 (s, 2H), 3.96 (s, 3H).

Step 2: To a solution of Intermediate 15-1 (1.00 g, 1.73 mmol, 1.00 eq.) and tributyl(2,3-dihydrofuran-5-yl)stannane (934 mg, 2.60 mmol, 1.50 eq.) in dioxane (20 mL) was added tetrakis[triphenylphosphine]palladium(0) (200 mg, 173 μmol, 0.10 eq.) at 20° C., the mixture was degassed and stirred at 100° C. for 16 hours under nitrogen atmosphere. The mixture was diluted with water (50 mL) and extracted with ethyl acetate (70.0 mL 3). The combined organic extracts were washed brine (50 mL), dried over anhydrous sodium sulfate, filtered and concentrated in vacuum and the residue was purified by flash silica gel chromatography (Petroleum ether/Ethyl acetate 0-40/6), to give 2-[4-[8-(2,3-dihydrofuran-5-yl)-4-[(1,3-dioxoisoindolin-2-yl)methyl]-1-oxo-2H-phthalazin-6-yl]-2-methyl-pyrazol-3-yl]benzothiophene-3-carbonitrile (580 mg, 55% yield) as a yellow solid. LCMS [M+1]⁺: 611.1. ¹H NMR (400 MHz, CDCl₃-d) δ=8.11-8.02 (m, 1H), 8.01-7.97 (m, 1H), 7.93 (d, J=8.0 Hz, 1H), 7.89-7.83 (m, 2H), 7.76-7.71 (m, 2H), 7.63-7.63 (m, 1H), 7.63-7.52 (m, 3H), 7.51-7.43 (m, 1H), 4.86-4.81 (m, 1H), 4.77 (s, 1H), 4.41 (t, J=9.2 Hz, 1H), 4.05-3.98 (m, 3H), 3.65 (t, J=5.6 Hz, 1H), 2.78-2.58 (m, 2H), 1.92-1.80 (m, 1H).

Step 3: To a solution of 2-[4-[8-(2,3-dihydrofuran-5-yl)-4-[(1,3-dioxoisoindolin-2-yl)methyl]-1-oxo-2H-phthalazin-6-yl]-2-methyl-pyrazol-3-yl]benzothiophene-3-carbonitrile (200 mg, 328 μmol, 1.00 eq.) in methanol (4.00 mL) and THF (8.00 mL) was added palladium on carbon (200 mg, 164 μmol, 10.0/6 purity) and triethylamine (99.4 mg, 983 μmol, 137 μL, 3.00 eq.) under nitrogen, the mixture was stirred at 40° C. for 16 hours under hydrogen (15 psi). The mixture was filtered, the filtrate concentrated in vacuum to give compound 2-[4-[4-[(1,3-dioxoisoindolin-2-yl)methyl]-1-oxo-8-THF-2-yl-2H-phthalazin-6-yl]-2-methyl-pyrazol-3-yl]benzothiophene-3-carbonitrile (140 mg, crude) as white solid. LCMS [M+1]⁺: 613.1

Step 4: 2-(4-(4-((1,3-dioxoisoindolin-2-yl)methyl)-1-oxo-8-(THF-2-yl)-1,2-dihydrophthalazin-6-yl)-1-methyl-1H-pyrazol-5-yl)benzo[b]thiophene-3-carbonitrile (140 mg, 229 μmol, 1.00 eq.) was separated by SFC (column: DAICEL CHIRALPAK AD (250 mm×30 mm, 10 μm); mobile phase: [0.1% NH₃·H₂O, IPA]; B %: 50%-50%, 4.6; 45 min), to give two compounds. Peak 1:2-(4-(4-((1,3-dioxoisoindolin-2-yl)methyl)-1-oxo-8-[(2S)-THF-2-yl]-1,2-dihydrophthalazin-6-yl)-1-methyl-1H-pyrazol-5-yl)benzo[b]thiophene-3-carbonitrile (60.0 mg, 42.2% yield) and Peak 2:2-(4-(4-((1,3-dioxoisoindolin-2-yl)methyl)-1-oxo-8-[(2R)-THF-2-yl]-1,2-dihydrophthalazin-6-yl)-1-methyl-1H-pyrazol-5-yl)benzo[b]thiophene-3-carbonitrile (80.0 mg, 57% yield) were obtained as white solids. LCMS [M+1]⁺: 613.2

Step 5: To a solution of 2-(4-(4-((1,3-dioxoisoindolin-2-yl)methyl)-1-oxo-8-[(2S)-THF-2-yl]-1,2-dihydrophthalazin-6-yl)-1-methyl-1H-pyrazol-5-yl)benzo[b]thiophene-3-carbonitrile (40.0 mg, 65.3 μmol, 1.00 eq.) in ethanol (1.0 mL) and THF (1.0 mL) was added hydrazine hydrate (35.0 mg, 685 μmol, 34.0 μL, 98.0% purity, 10.5 eq.) in ethanol (0.50 mL) at 20° C., then the mixture was stirred at 55° C. for 1 hour. The reaction mixture was concentrated under reduced pressure and the residue was purified by prep-HPLC (column: Welch Xtimate C18 150×25 mm×5 μm; mobile phase: [water (0.05% HCl)-ACN]; B %: 12%-42%, 10 min), to give (S)-2-(4-(4-(aminomethyl)-1-oxo-8-(THF-2-yl)-1,2-dihydrophthalazin-6-yl)-1-methyl-1H-pyrazol-5-yl)benzo[b]thiophene-3-carbonitrile, Example 15-3 (10 mg, 30% yield) as a white solid. LCMS [M+1]⁺: 483.0. ¹H NMR (400 MHz, MeOD-d₄) δ=8.24 (s, 1H), 8.20-8.12 (m, 1H), 8.05-7.97 (m, 1H), 7.95-7.85 (m, 1H), 7.74 (d, J=1.6 Hz, 2H), 7.64-7.58 (m, 1H), 5.98-5.98 (m, 1H), 6.07-5.80 (m, 1H), 4.62-4.48 (m, 2H), 3.99 (s, 3H), 3.40 (q, J=8.0 Hz, 1H), 3.01-2.91 (m, 1H), 2.54-2.39 (m, 1H), 1.60-1.48 (m, 1H), 1.38-1.26 (m, 1H), 1.21-1.06 (m, 1H). 4 Example 15-4 was obtained from 2-(4-(4-((1,3-dioxoisoindolin-2-yl)methyl)-1-oxo-8-[(2R)-THF-2-yl]-1,2-dihydrophthalazin-6-yl)-1-methyl-1H-pyrazol-5-yl)benzo[b]thiophene-3-carbonitrile (80.0 mg, 131 μmol.) in a same fashion to give (R)-2-(4-(4-(aminomethyl)-1-oxo-8-(THF-2-yl)-1,2-dihydrophthalazin-6-yl)-1-methyl-1H-pyrazol-5-yl)benzo[b]thiophene-3-carbonitrile, Example 15-4 (30 mg, 45% yield) as a yellow solid. LCMS [M+1]⁺: 483.3. ¹H NMR (400 MHz, MeOD-d₄) δ=8.24 (s, 1H), 8.16 (dd, J=2.0, 6.8 Hz, 1H), 8.01 (dd, J=2.0, 6.4 Hz, 1H), 7.90 (s, 1H), 7.74-7.64 (m, 2H), 7.62 (s, 1H), 6.02-5.89 (m, 1H), 4.64-4.46 (m, 2H), 3.99 (s, 3H), 3.40 (d, J=6.8 Hz, 1H), 3.01-2.89 (m, 1H), 2.55-2.38 (m, 1H), 1.61-1.48 (m, 1H), 1.37-1.27 (m, 1H), 1.21-1.08 (m, 1H).

### Example 15-5

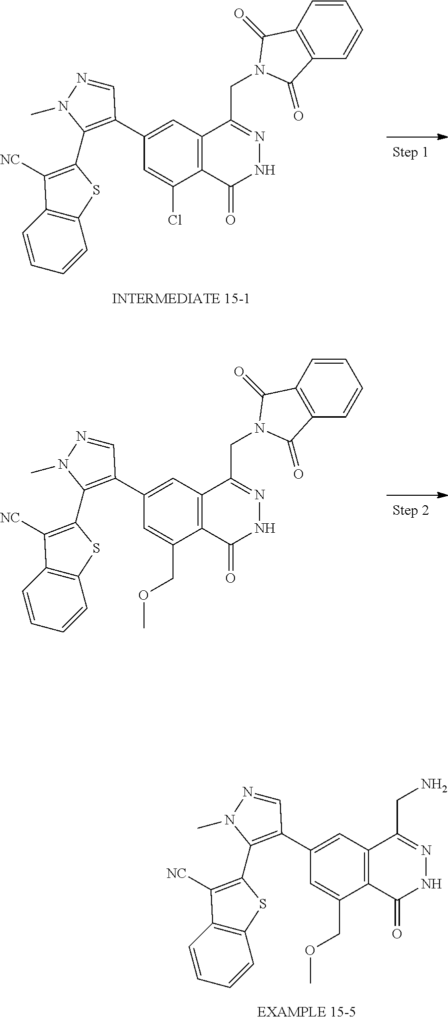 [SMILES: Cn1ncc(-c2cc(Cl)c3c(=O)[nH]nc(CN4C(=O)c5ccccc5C4=O)c3c2)c1-c1sc2ccccc2c1C#N ; COCc1cc(-c2cnn(C)c2-c2sc3ccccc3c2C#N)cc2c(CN3C(=O)c4ccccc4C3=O)n[nH]c(=O)c12 ; COCc1cc(-c2cnn(C)c2-c2sc3ccccc3c2C#N)cc2c(CN)n[nH]c(=O)c12]

Step 1: To a solution of tributyl(methoxymethyl)stannane (600 mg, 298 μmol, 9.00 eq), Intermediate 15-1 (115 mg, 199 μmol, 1 eq) in dioxane (3.0 mL) was added tetrakis[triphenylphosphine]palladium(0) (69.0 mg, 59.7 μmol, 0.30 eq) at 20° C., the mixture was degassed and stirred at 100° C. for 16 hours under nitrogen. The mixture was diluted with ethyl acetate (100 mL 3), water (80 mL). The combined organic extracts were washed brine (80 mL) and then dried over anhydrous sodium sulfate, filtered and concentrated in vacuum and the residue was purified by prep-TLC (silicon dioxide, Dichloromethane/Ethyl acetate 3:1). To give compound 2-[4-[4-[(1,3-dioxoisoindolin-2-yl)methyl]-8-(methoxymethyl)-1-oxo-2H-phthalazin-6-yl]-2-methyl-pyrazol-3-yl]benzothiophene-3-carbonitrile (25 mg, 14% yield, 63% purity) as a white solid. LC-MS [M+1]⁺: 587.3

Step 2: To a solution of 2-[4-[4-[(1,3-dioxoisoindolin-2-yl)methyl]-8-(methoxymethyl)-1-oxo-2H-phthalazin-6-yl]-2-methyl-pyrazol-3-yl]benzothiophene-3-carbonitrile (13.0 mg, 22.2 μmol, 1.00 eq) in ethanol (0.50 mL) and THF (0.50 mL) was added hydrazine hydrate (11.3 mg, 222 μmol, 11.0 μL, 10.0 eq) in ethanol (0.10 mL) at 20° C., then the mixture was stirred at 55° C. for 1 hour. The reaction mixture was concentrated under reduced pressure and the reside was purified by perp-HPLC (column: Welch Xtimate C18 150×25 mm×5 μm; mobile phase: [water (0.05% HCl)-ACN]; B %: 11%-41%, 10 min), to give compound 2-[4-[4-(aminomethyl)-8-(methoxymethyl)-1-oxo-2H-phthalazin-6-yl]-2-methyl-pyrazol-3-yl]benzothiophene-3-carbonitrile (2 mg, 19% yield) as an off-white solid. LCMS [M+1]⁺: 457.2. ¹H NMR (400 MHz, MeOD-d₄) δ=8.23 (s, 1H), 8.16-8.14 (m, 1H), 8.02-8.00 (m, 1H), 7.84 (d, J=1.6 Hz, 1H), 7.74 (d, J=1.2 Hz, 1H), 7.69-7.67 (m, 2H), 4.94 (s, 2H), 4.47 (s, 2H), 3.98 (s, 3H), 2.91 (s, 3H).

### Example 15-6

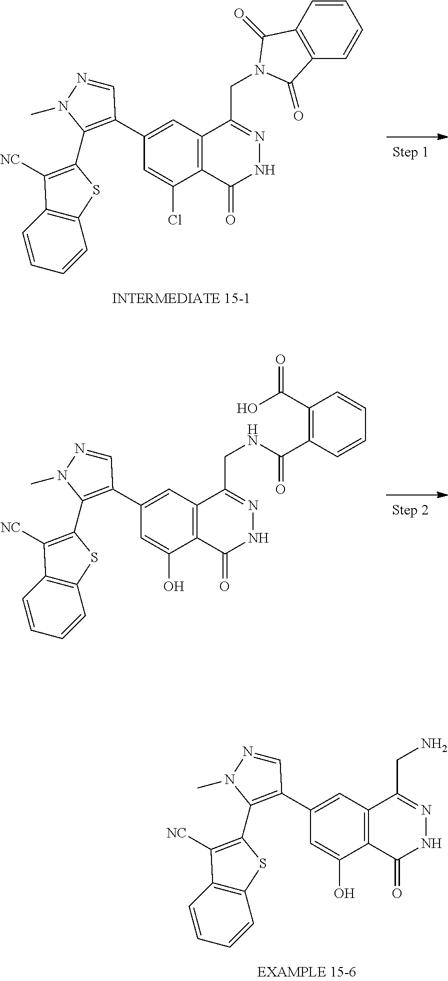 [SMILES: Cn1ncc(-c2cc(Cl)c3c(=O)[nH]nc(CN4C(=O)c5ccccc5C4=O)c3c2)c1-c1sc2ccccc2c1C#N ; Cn1ncc(-c2cc(O)c3c(=O)[nH]nc(CNC(=O)c4ccccc4C(=O)O)c3c2)c1-c1sc2ccccc2c1C#N ; Cn1ncc(-c2cc(O)c3c(=O)[nH]nc(CN)c3c2)c1-c1sc2ccccc2c1C#N]

Step 1: To a solution of Intermediate 15-1 (150 mg, 260 μmol, 1.00 eq.) and N,N-bis(4-hydroxy-2,6-dimethyl-phenyl)oxamide (4 mg, 13.0 μmol, 0.05 eq.) in dimethylsulfoxide (2.5 mL) and water (0.6 mL) was added lithium hydroxide (23 mg, 546 μmol, 2.10 eq.) and bis[(Z)-1-methyl-3-oxo-but-1-enoxy]copper (3.40 mg, 13.0 μmol, 0.05 eq.). The mixture was stirred at 120° C. for 16 hrs. The mixture was filtered. The filtrate was purified by prep-HPLC (column: Shim-pack C18 150×25×10 μm; mobile phase: [water (0.225% formic acid)-acetonitrile]; B %: 39/6-57%, 9 min). The eluted fraction containing [M+1]⁺ 577.2 was extracted with ethyl acetate (10 mL×3). The combined organic phase was washed with brine (20 mL), dried over anhydrous sodium sulfate, filtered and concentrated to afford 2-(((7-(5-(3-cyanobenzo[b]thiophen-2-yl)-1-methyl-1H-pyrazol-4-yl)-5-hydroxy-4-oxo-3,4-dihydrophthalazin-1-yl)methyl)carbamoyl)benzoic acid (21 mg, 26.2 μmol, formic acid salt) as a yellow solid. LCMS [ESI, M+1]⁺: 577.2.

Step 2: To a solution of 2-(((7-(5-(3-cyanobenzo[b]thiophen-2-yl)-1-methyl-1H-pyrazol-4-yl)-5-hydroxy-4-oxo-3,4-dihydrophthalazin-1-yl)methyl)carbamoyl)benzoic acid (20.0 mg, 32.1 μmol, 1.00 eq., formic acid salt) in ethanol (0.80 mL) was added hydrazine hydrate (16.1 mg, 321 μmol, 15.6 μL, 10.0 eq.). The mixture was stirred at 80° C. for 3 hours. The mixture was concentrated. The residue was purified by prep-HPLC (column: 3_Phenomenex Luna C18 75×30 mm×3 μm; mobile phase: [water (0.05% hydrochloric acid)-acetonitrile]; B %: 20%-40%, 6.5 min) to afford 2-(4-(4-(aminomethyl)-8-hydroxy-1-oxo-1,2-dihydrophthalazin-6-yl)-1-methyl-1H-pyrazol-5-yl)benzo[b]thiophene-3-carbonitrile (4 mg, 26% yield) as an off-white solid. LCMS [M+1]⁺: 429.1. ¹H NMR (400 MHz, CD₃OD) δ=8.16-8.11 (m, 2H), 8.01 (dd, J=2.4, 7.2 Hz, 1H), 7.72-7.62 (m, 2H), 7.24 (d, J=1.2 Hz, 1H), 6.92 (d, J=1.2 Hz, 1H), 4.30 (s, 2H), 3.97 (s, 3H).

### Example 15-7

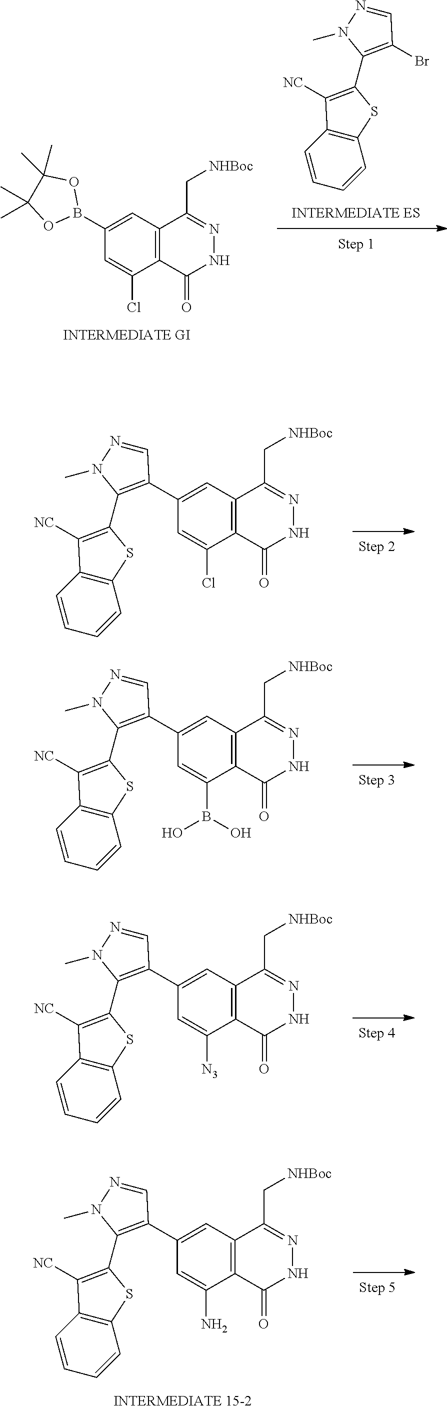

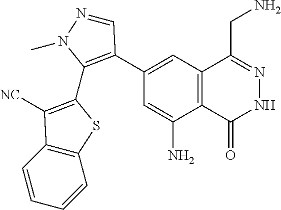 [SMILES: Cn1ncc(-c2cc(N)c3c(=O)[nH]nc(CN)c3c2)c1-c1sc2ccccc2c1C#N]

Step 1: To a mixture of Intermediate GI (1.64 g, 3.77 mmol, 1.20 eq.) and Intermediate ES (1.00 g, 3.14 mmol, 1.00 eq.) in dioxane (30 mL) and water (6.0 mL) was added sodium carbonate (833 mg, 7.86 mmol, 2.50 eq.) and Pd(dppf)Cl₂ (230 mg, 314 μmol, 0.10 eq.). The mixture was degassed and stirred at 80° C. for 3 hours under nitrogen atmosphere. The mixture was diluted with water (200 mL) and extracted with ethyl acetate (50 mL×3). The organic layer was washed with brine (100 mL), dried over anhydrous sodium sulfate, filtered and concentrated in vacuum. The residue was purified by flash silica gel chromatography (Petroleum ether/Ethyl acetate 10-100%), to afford tert-butyl ((5-chloro-7-(5-(3-cyanobenzo[b]thiophen-2-yl)-1-methyl-1H-pyrazol-4-yl)-4-oxo-3,4-dihydrophthalazin-1-yl)methyl)carbamate (1.00 g, 58% yield) as light a yellow solid. LCMS [M+1]⁺: 547.2. ¹H NMR (400 MHz, CDCl₃) δ=9.91 (br s, 1H), 8.09-7.91 (m, 3H), 7.85 (br s, 1H), 7.69-7.56 (m, 2H), 7.46 (s, 1H), 5.29-5.10 (m, 1H), 4.39 (br d, J=4.4 Hz, 2H), 3.99 (s, 3H), 1.47 (s, 9H).

Step 2: To a solution of tert-butyl ((5-chloro-7-(5-(3-cyanobenzo[b]thiophen-2-yl)-1-methyl-1H-pyrazol-4-yl)-4-oxo-3,4-dihydrophthalazin-1-yl)methyl)carbamate (1.50 g, 2.74 mmol, 1.00 eq.) and 4,4,5,5-tetramethyl-2-(4,4,5,5-tetramethyl-1,3,2-dioxaborolan-2-yl)-1,3,2-dioxaborolane (1.04 g, 4.11 mmol, 1.50 eq.) in dioxane (30 mL) was added potassium acetate (673 mg, 6.86 mmol, 2.50 eq.) and Pd(dppf)Cl₂ (201 mg, 274 μmol, 0.10 eq.). Then the mixture was stirred at 100° C. for 16 hours under nitrogen atmosphere. The mixture was concentrated. The residue was diluted with dimethylsulfoxide (10.0 mL) and was purified by prep-HPLC (column: Phenomenex luna C18 150×25 mm×10 μm; mobile phase: [water (0.2% formic acid)-acetonitrile]; B %: 34%-64%, 12 min) and lyophilzed to afford (1-(((tert-butoxycarbonyl)amino)methyl)-7-(5-(3-cyanobenzo[b]thiophen-2-yl)-1-methyl-1H-pyrazol-4-yl)-4-oxo-3,4-dihydrophthalazin-5-yl)boronic acid (300 mg, 19% yield,) as a white solid. LCMS [M+1]⁺: 557.2. ¹H NMR (400 MHz, DMSO-d₆) δ=12.62-12.46 (m, 1H), 8.31-8.23 (m, 1H), 8.20 (s, 1H), 8.05-7.95 (m, 2H), 7.90 (s, 2H), 7.72-7.63 (m, 2H), 7.51 (s, 1H), 7.32 (br t, J=5.2 Hz, 1H), 4.23 (br d, J=5.6 Hz, 2H), 3.93-3.87 (m, 3H), 1.37 (s, 9H).

Step 3: To a solution of [1-[(tert-butoxycarbonylamino)methyl]-7-[5-(3-cyanobenzothiophen-2-yl)-1-methyl-pyrazol-4-yl]-4-oxo-3H-phthalazin-5-yl]boronic acid (150 mg, 269.59 μmol, 1 eq) and NaN₃ (26.3 mg, 404 μmol, 1.50 eq.) in methanol (3.0 mL) was added copper acetate (4.90 mg, 27.0 μmol, 0.10 eq.). Then the reaction was stirred at 45° C. for 16 hours under oxygen (15 psi) atmosphere. The mixture was diluted with methanol (15 mL). The mixture was filtered. The filtrate was diluted with water (2.0 mL) and the mixture was concentrated to 2.0 mL to afford tert-butyl ((5-azido-7-(5-(3-cyanobenzo[b]thiophen-2-yl)-1-methyl-1H-pyrazol-4-yl)-4-oxo-3,4-dihydrophthalazin-1-yl)methyl)carbamate (120 mg, crude, dissolved in water) as a yellow liquid. LCMS [M+1]⁺: 528.2.

Step 4: To a solution of tert-butyl ((5-azido-7-(5-(3-cyanobenzo[b]thiophen-2-yl)-1-methyl-1H-pyrazol-4-yl)-4-oxo-3,4-dihydrophthalazin-1-yl)methyl)carbamate (100 mg, 181 μmol, 1.00 eq.) in THF (6.0 mL) and water (2.0 mL) was added triphenylphosphine (284 mg, 1.08 mmol, 6.00 eq.). Then the mixture was stirred at 55° C. for 2 hours. The mixture was concentrated. The residue was purified by prep-HPLC (column: Waters Xbridge 150×25 mm×5 μm; mobile phase: [water (0.05% ammonia hydroxide v/v)-acetonitrile];B %: 34/6-64%, 8 min) and then SFC (column: DAICEL CHIRALCEL OD (250 mm×30 mm, 10 μm); mobile phase: [0.1% ammonium hydroxide isopropanol]; B %: 45/6-45%, 2.9 min; 40 min) to afford tert-butyl ((5-amino-7-(5-(3-cyanobenzo[b]thiophen-2-yl)-1-methyl-1H-pyrazol-4-yl)-4-oxo-3,4-dihydrophthalazin-1-yl)methyl)carbamate, Intermediate 15-2 (35 mg, 37% yield) as a white solid. LCMS [M+1]⁺: 528.2. ¹H NMR (400 MHz, DMSO-d₆) δ=12.09 (br s, 1H), 8.31-8.18 (m, 1H), 8.08-7.96 (m, 2H), 7.66 (m, 2H), 7.37-7.14 (m, 3H), 6.99 (s, 1H), 6.59 (s, 1H), 4.13 (br d, J=5.6 Hz, 2H), 3.88 (s, 3H), 1.38 (s, 9H).

Step 5: To a solution of Intermediate 15-2 (10 mg, 19.0 μmol, 1.00 eq.) in methanol (0.3 mL) was added hydrochloric acid/methanol (4 M, 0.3 mL, 63.3 eq.) and the mixture was stirred at 20° C. for 3 hours. The mixture was concentrated. The residue was purified by prep-HPLC (Phenomenex Luna C18 75×30 mm×3 μm, mobile phase: [water (0.05% hydrochloric acid)-acetonitrile]; B %: 17%-37%, 6 min) and lyophilized to afford 2-(4-(8-amino-4-(aminomethyl)-1-oxo-1,2-dihydrophthalazin-6-yl)-1-methyl-1H-pyrazol-5-yl)benzo[b]thiophene-3-carbonitrile (6 mg, 65% yield) as a white solid. LCMS [M+1]⁺: 428.3. ¹H NMR (400 MHz, METHANOL-d₄) δ=8.11 (br d, J=7.6 Hz, 1H), 8.02 (s, 2H), 7.73-7.59 (m, 2H), 6.78 (s, 1H), 6.71 (s, 1H), 4.13 (s, 2H), 3.96 (s, 3H).

### Example 15-8

Example 15-8 is obtained from Intermediate 15-2 via acetylation reaction with acetyl chloride (3 eq.) in presence of triethylamine (3 eq.) in DCM followed by removal of Boc-protecting group by reacting with HCl in Methanol.

### Example 15-9

Example 15-9 is obtained from Intermediate 15-1 via Pd-catalyzed borylation then Pd-catalyzed reaction with 2-(difluoromethylene)-1,1,1-trifluoro-3,5,8-trioxa-1□⁴-boranonane, potassium salt, followed by removal of Boc-protecting group by reacting with HCl in Methanol.

### Examples 15-10 and 15-11

Examples 15-10 and 15-11 are prepared in the similar reaction sequence as Examples 15-3 and 15-4 from Intermediate 15-1.

TABLE 15

| Example | Structure | Compound Name |
|---|---|---|
| 15-8 | 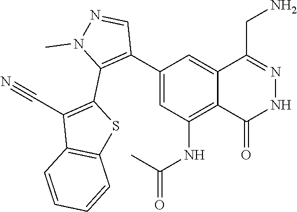 [SMILES: CC(=O)Nc1cc(-c2cnn(C)c2-c2sc3ccccc3c2C#N)cc2c(CN)n[nH]c(=O)c12] | N-(1-(aminomethyl)-7-(5-(3-cyano- benzo[b]thiophen-2-yl)-1-methyl-1H- pyrazol-4-yl)-4-oxo-3,4-dihydrophthal- azin-5-yl)acetamide |
| 15-9 | 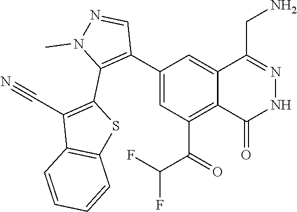 [SMILES: Cn1ncc(-c2cc(C(=O)C(F)F)c3c(=O)[nH]nc(CN)c3c2)c1-c1sc2ccccc2c1C#N] | 2-(4-(4-(aminomethyl)-8-(2,2-difluoro- acetyl)-1-oxo-1,2-dihydrophthalazin-6- yl)--methyl-1H-pyrazol-5-yl)benzo[b]- thiophene-3-carbonitrile |
| 15-10 | 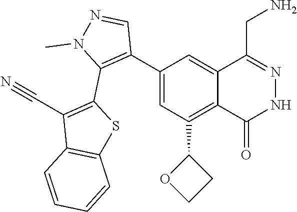 [SMILES: Cn1ncc(-c2cc([C@@H]3CCO3)c3c(=O)[nH]nc(CN)c3c2)c1-c1sc2ccccc2c1C#N] | (S)-2-(4-(4-(aminomethyl)-8-(oxctan-2- yl)-1-oxo-1,2-dihydrophthalazin-6-yl)-1- methyl-1H-pyrazol-5-yl)benzo[b]thio- phene-3-carbonitrile |
| 15-11 | 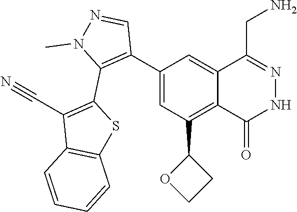 [SMILES: Cn1ncc(-c2cc([C@H]3CCO3)c3c(=O)[nH]nc(CN)c3c2)c1-c1sc2ccccc2c1C#N] | (R)-2-(4-(4-(aminomethyl)-8-(oxetan-2- yl)-1-oxo-1,2-dihydrophthalazin-6-yl)-1- methyl-1H-pyrazol-5-yl)benzo[b]thio- phene-3-carbonitrile |

### Example 16-1 & 16-2

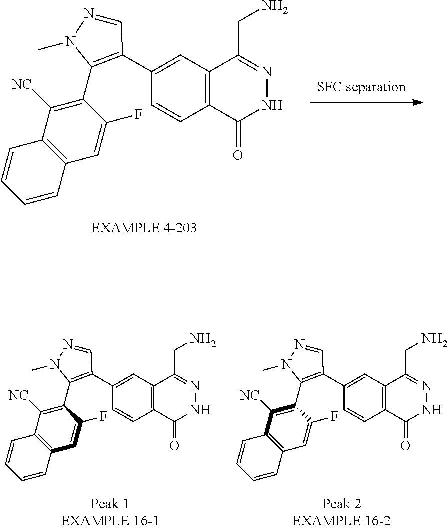

Example 4-203 (30 mg, 0.071 mmol) was separated by SFC (Daicel Chiralpak AD (250 mm×30 mm, 10 μm); mobile phase: [0.1% NH₃H₂O EtOH]; B %: 40/6-40%%, 4.9 min; 40 min) to provide Example 16-1 (6 mg, 0.014 mmol, 19% yield) as a white solid LCMS [M+1]⁺=425.2; ¹H NMR (400 MHz, CD₃OD) δ=8.20-8.15 (m, 1H), 8.13-8.03 (m, 4H), 7.76-7.67 (m, 2H), 7.59 (d, J=1.2 Hz, 1H), 7.49 (dd, J=1.6, 8.4 Hz, 1H), 3.85-3.79 (m, 1H), 3.76 (s, 3H), 3.75-3.70 (m, 1H) and Example 16-2 (6 mg, 0.014 mmol, 19% yield) as a white solid LCMS [M+1]⁺=425.2; ¹H NMR (400 MHz, CD₃OD) δ=8.18 (d, J=10.0 Hz, 1H), 8.13 (s, 1H), 8.12-8.02 (m, 3H), 7.79-7.67 (m, 3H), 7.36 (dd, J=1.2, 8.4 Hz, 1H), 4.44-4.08 (m, 2H), 3.77 (s, 3H).

### Example 16-3 and 16-4

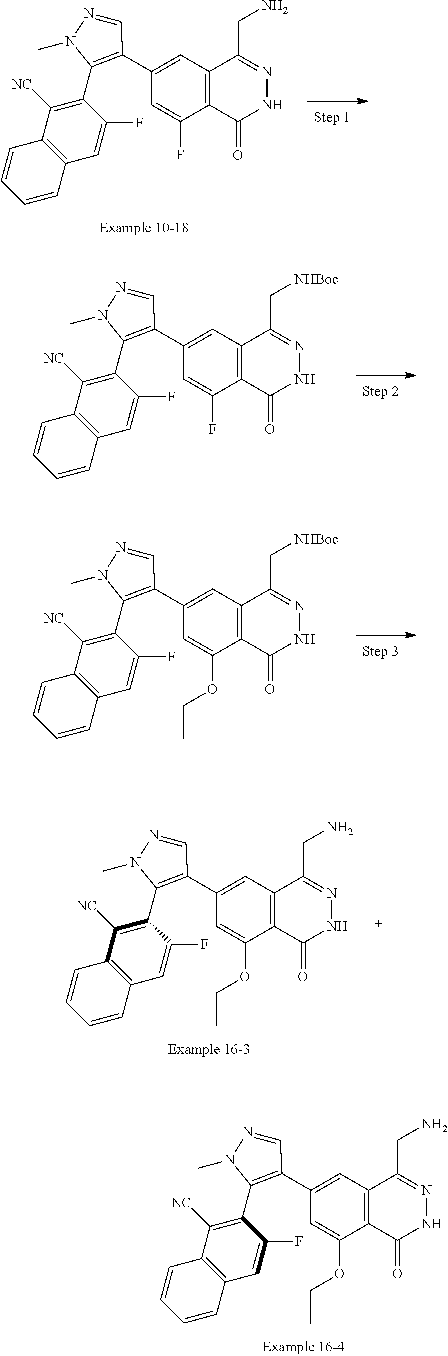

Step 1: To a solution of 2-[4-[4-(aminomethyl)-8-fluoro-1-oxo-2H-phthalazin-6-yl]-2-methyl-pyrazol-3-yl]-3-fluoro-naphthalene-1-carbonitrile (35.0 mg, 0.079 mmol, 1.00 eq.) in THF (4 mL) was added Boc₂O (17 mg, 0.079 mmol, 1.00 eq.) and the mixture was stirred at rt for 12 hrs. The reaction mixture was then concentrated and the residue purified by prep-TLC (SiO₂, dichloromethane:methanol 10%) to give tert-butyl N-[[7-[5-(1-cyano-3-fluoro-2-naphthyl)-1-methyl-pyrazol-4-yl]-5-fluoro-4-oxo-3H-phthalazin-1-yl]methyl]carbamate (30 mg, 0.055 mmol, 70% yield) as a yellow solid. LCMS [M+1]⁺=543.2.

Step 2: To a solution of tert-butyl N-[[7-[5-(1-cyano-3-fluoro-2-naphthyl)-1-methyl-pyrazol-4-yl]-5-fluoro-4-oxo-3H-phthalazin-1-yl]methyl]carbamate (30 mg, 0.055 mmol, 1.00 eq.) in alcohol (2 mL) was added sodium ethoxide (19 mg, 0.276 mmol, 5.00 eq.) and the mixture was stirred at 110° C. for 0.75 hr in a sealed tube. The cooled reaction mixture was then filtered, concentrated and the residue triturated with MTBE (1 mL), filtered and dried to give compound tert-butyl N-[[7-[5-(1-cyano-3-fluoro-2-naphthyl)-1-methyl-pyrazol-4-yl]-5-ethoxy-4-oxo-3H-phthalazin-1-yl]methyl]carbamate (30 mg, 0.052 mmol, 96% yield) as a yellow solid. LCMS [M+1]⁺=569.3.

Step 3: To a solution of tert-butyl N-[[7-[5-(1-cyano-3-fluoro-2-naphthyl)-1-methyl-pyrazol-4-yl]-5-ethoxy-4-oxo-3H-phthalazin-1-yl]methyl]carbamate (30 mg, 0.052 mmol, 1.00 eq.) in dichloromethane (0.2 mL) was added TFA (0.75 mL) and the mixture was stirred at 30° C. for 0.25 hr. The reaction mixture was then concentrated and the residue purified by prep-HPLC (purification method 4-6) then sterosiomers were separated by SFC (DAICEL CHIRALPAK IG (250 mm×30 mm, 10 μm); mobile phase: [Heptane-alcohol (0.1% NH₃H₂O)]; B %: 80% isocratic; 7 min cycle, 150 min total) then each stereosiomer was further purified by prep-HPLC (Unisil 3-100 C18 ultra 150 mm×50 mm×3 μm; mobile phase: [water (0.225% formic acid)-ACN]; B %: 15%-35%, 10 min) to give example 16-3, 2-[4-[4-(aminomethyl)-8-ethoxy-1-oxo-2H-phthalazin-6-yl]-2-methyl-pyrazol-3-yl]-3-fluoro-naphthalene-1-carbonitrile (1.4 mg, 0.002 mmol, 20% yield,) as a white solid and example 16-4 2-[4-[4-(aminomethyl)-8-ethoxy-1-oxo-2H-phthalazin-6-yl]-2-methyl-pyrazol-3-yl]-3-fluoro-naphthalene-1-carbonitrile (1.7 mg, 0.003 mmol, 25% yield,) as a white solid. Spectra data for Example 16-3: LCMS [M+1]⁺=569.3; ¹H NMR (400 MHz, MeOD) 5=8.53 (br s, 2H), 8.33 (d, J=10.0 Hz, 1H), 8.27 (s, 1H), 8.25-8.18 (m, 2H), 7.90-7.84 (m, 2H), 7.35 (s, 1H), 6.83 (s, 1H), 4.38-4.19 (m, 2H), 3.90 (s, 3H), 3.77-3.66 (m, 1H), 3.60-3.48 (m, 1H), 1.03 (t, J=6.8 Hz, 3H). Spectra data for Example 16-4: LCMS [M+1]⁺=569.3; ¹H NMR (400 MHz, CD₃OD) δ=8.59-8.47 (m, 1H), 8.31 (d, J=10.0 Hz, 1H), 8.25 (s, 1H), 8.24-8.16 (m, 2H), 7.88-7.81 (m, 2H), 7.32 (s, 1H), 6.82 (s, 1H), 4.33-4.13 (m, 2H), 3.88 (s, 3H), 3.76-3.64 (m, 1H), 3.59-3.46 (m, 1H), 1.01 (t, J=6.8 Hz, 3H).

### Example 16-5 and 16-6

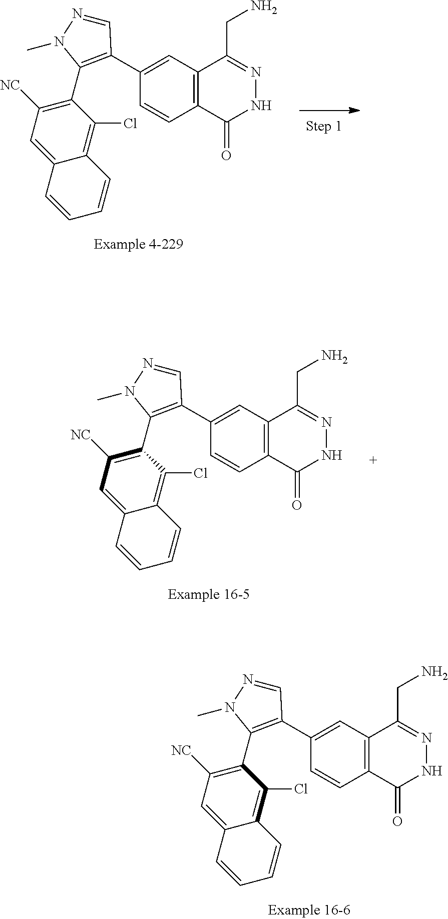

Example 16-5 (49 mg, 0.112 mmol, 31% yield) and Example 16-6 (48 mg, 0.109 mmol, 30% yield) was separated from Example 4-229 (160 mg, 0.324 mmol) by SFC. MeOH (20 ml) was added to the sample. Waters 80Q instrument, Supercritical CO₂ Flow Rate: 80 g/min Cycle Time: 7 min, total time: 50 min. Single injection volume: 3.0 ml. Back Pressure: 100 bar to keep the CO₂ in Supercritical flow. Phenomenex-Cellulose-2 (250 mm×30 mm, 10 μm); mobile phase: [0.1% NH₃H₂O MeOH]; B %: 60% isocratic). Spectra data for Example 16-5: LCMS [M+1]⁺=441.2; ¹H NMR (400 MHz, DMSO-d₆) δ=12.82 (s, 1H), 8.94 (s, 1H), 8.48 (s, 1H), 8.44-8.39 (m, 1H), 8.32 (br d, J=8.0 Hz, 1H), 8.05-7.99 (m, 2H), 7.98-7.89 (m, 2H), 7.26 (br d, J=8.4 Hz, 1H), 4.35 (br s, 2H), 3.71 (s, 3H). Spectra data for Example 16-5: LCMS [M+1]⁺=441.2; ¹H NMR (400 MHz, DMSO-d₆) δ=12.39 (br s, 1H), 8.92 (s, 1H), 8.42 (d, J=8.4 Hz, 1H), 8.34 (s, 1H), 8.30 (d, J=8.0 Hz, 1H), 8.07 (d, J=8.4 Hz, 1H), 8.06-8.00 (m, 1H), 7.98-7.92 (m, 1H), 7.67 (d, J=1.6 Hz, 1H), 7.64 (dd, J=1.6, 8.4 Hz, 1H), 3.73 (s, 3H), 3.54 (s, 2H).

### Example 16-7 and 16-8

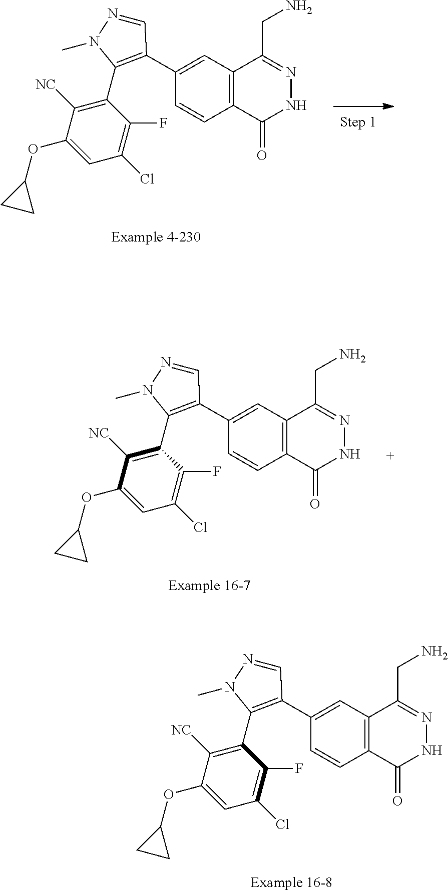

Example 4-230, 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (30 mg, 0.065 mmol) separated by SFC (DAICEL CHIRALPAK IC (250 mm×30 mm×10 μm); mobile phase: [0.1% NH₃H₂O isopropanol]; B %: 40% isocratic, 4.1 min cycle; 120 min total) to give example 16-7 (ee>99/a, 13 mg, 0.026 mmol, 25% yield) as a yellow solid and example 16-8 (8 mg, ee=84%). Example 16-8 was then then further separated by SFC (DAICEL CHIRALPAK IC (250 mm×30 mm×10 m); mobile phase: [0.1% NH₃H₂O EtOH]; B %: 60% isocratic, 3.1 min cycle; total 50 min) to give Example 16-8 (ee>99%, 4 mg, 0.007 mmol, 7% yield) as a yellow gum. Spectra data for Example 16-7: LCMS [M+1]⁺=465.1; ¹H NMR (400 MHz, DMSO-d₆) δ=12.59-12.44 (s, 1H), 8.29 (s, 1H), 8.15 (d, J=8.4 Hz, 1H), 8.01 (d, J=6.0 Hz, 1H), 7.75 (s, 1H), 7.67 (br d, J=7.6 Hz, 1H), 4.23-4.17 (m, 1H), 3.86 (br s, 2H), 3.78 (s, 3H), 0.94-0.88 (m, 2H), 0.84-0.79 (m, 2H). Spectra data for Example 16-8: LCMS [M+1]⁺=465.1; ¹H NMR (400 MHz, DMSO-d₆) δ=12.49-12.37 (s, 1H), 8.26 (s, 1H), 8.15 (d, J=8.4 Hz, 1H), 8.00 (d, J=6.0 Hz, 1H), 7.73 (d, J=1.6 Hz, 1H), 7.72-7.68 (m, 1H), 4.19 (m, 1H), 3.80 (s, 2H), 3.77 (s, 3H), 0.93-0.87 (m, 2H), 0.83-0.78 (m, 2H).

Coupling Method (CM) for the Preparation of Examples in Table 16 General Coupling Method CM-16-1

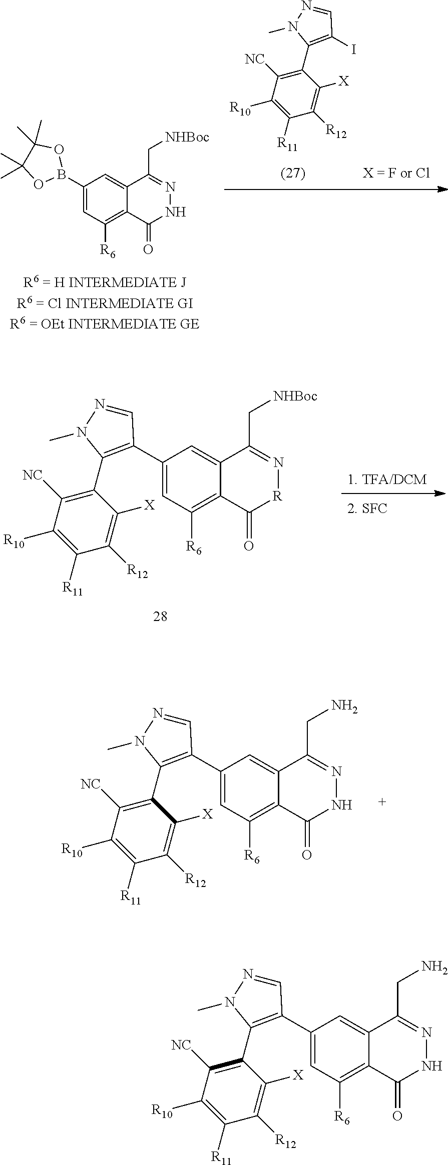

Step 1: A mixture of the appropriate aryl/heteroaryl-iodide 27 (390 μmol, 1.5 eq.), intermediate J or GI or GE (260 μmol, 1.00 eq.), palladium catalyst (See Table 16-CM) (26.0 μmol, 0.10 eq.), base (See Table 16-CM) (0.521 μmol, 2.0-3.0 eq.) in solvent (1.2 mL, See Table 16-CM) was purged with nitrogen 3 times. The mixture was then stirred at 80° C. for 2 hours. After such time the mixture was purified using the methods described for CM 10A-1 or CM 10A-2 to give coupling product 28.

Step 2: To a solution of corresponding coupling product 28 in DCM (2.0 mL) was added TFA (0.4 mL). The mixture was stirred at 25° C. for 0.5 hour. After such time the mixture was concentrated in vacuo and the residue purified by prep-HPLC according to one of the purification methods (PM) 4-1 through 4-23.

Step 3: The mixture of stereoisomers was separated by SFC purification (DAICEL CHIRALPAK IC (250 mm×30 mm×10 μm); mobile phase: [0.1% NH₃ water IPA];B %: isocratic)

### General Coupling Method CM-16-2

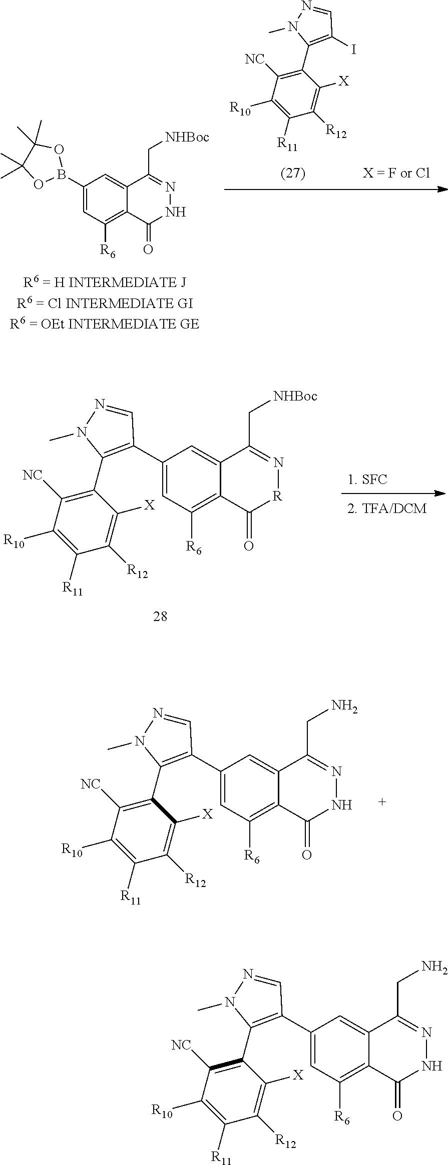

Step 1: A mixture of the appropriate aryl/heteroaryl-iodide 27 (390 μmol, 1.5 eq.), intermediate J or GI or GE (260 μmol, 1.00 eq.), palladium catalyst (See Table 16-CM) (26.0 μmol, 0.10 eq.), base (See Table 16-CM) (0.521 μmol, 2.0-3.0 eq.) in solvent (1.2 mL, See Table 16-CM) was purged with nitrogen 3 times. The mixture was then stirred at 80° C. for 2 hours. After such time the mixture was purified using the methods described for CM 10A-1 or CM 10A-2 to give coupling product 28.

Step 2: The mixture of stereoisomers 28 was separated by SFC purification (DAICEL CHIRALPAK IC (250 mm×30 mm, 10 μm); mobile phase: [0.1% NH₃ water [PA];B %: isocratic)

Step 3: To each of the separated isomers 28 in DCM (2.0 mL) was added TFA (0.4 mL). The mixture was stirred at 25° C. for 0.5 hour. After such time the mixture was concentrated in vacuo and the residue purified by prep-HPLC according to one of the purification methods (PM) 4-1 through 4-23.

### General Coupling Method CM-16-2*

Step 1 and Step 2 are identical to GENERAL COUPLING METHOD CM-16-2

Step 3: To each of the separated isomers 28 in dioxane (1.0 mL) was added 4M hydrochloric acid/dioxane (1.0 mL) or 1M hydrochloric acid/methyl alcohol (2 mL). The mixture was stirred at 20° C. for 2 hours. The mixture was concentrated. in vacuo and the residue purified according to one of the purification methods (PM) 4-1 through 4-23. GENERAL COUPLING METHOD CM-16-3

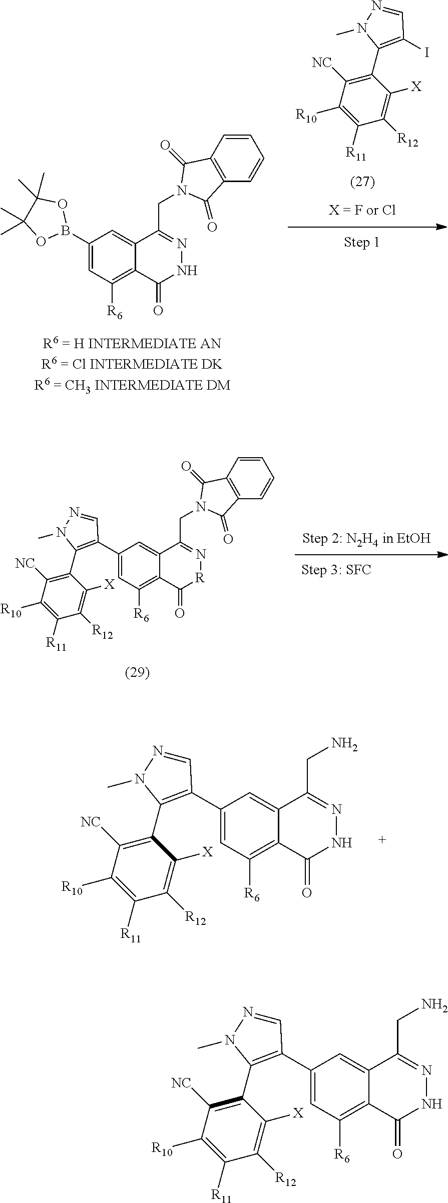

Step 1: A mixture of the appropriate aryl/heteroaryl-iodide 27 (390 μmol, 1.5 eq.), intermediate AN or DK or DM (260 μmol, 1.00 eq.), palladium catalyst (See Table 16-CM) (26.0 μmol, 0.10 eq.), base (See Table 16-CM) (0.521 μmol, 2.0-3.0 eq.) in solvent (1.2 mL, See Table 16-CM) was purged with nitrogen 3 times. The mixture was then stirred at 80° C. for 2 hours. After such time the mixture was purified using the methods described for CM 10A-1 or CM 10A-2 to give coupling product 29.

Step 2: To a solution of corresponding coupling product 29 in ethyl alcohol (1.0 mL) was added hydrazine hydrate (20 eq.). The mixture was stirred at 80° C. for 1 hour. After such time the mixture was concentrated in vacuo and the residue purified by prep-HPLC according to one of the purification methods (PM) 4-1 through 4-23.

Step 3: The mixture of stereoisomers was separated by SFC purification (column: DAICEL CHIRALPAK IC (250 mm 30 mm, 10 μm); mobile phase: [0.1% NH₃ water IPA];B %: isocratic)

### General Coupling Method CM-16-4

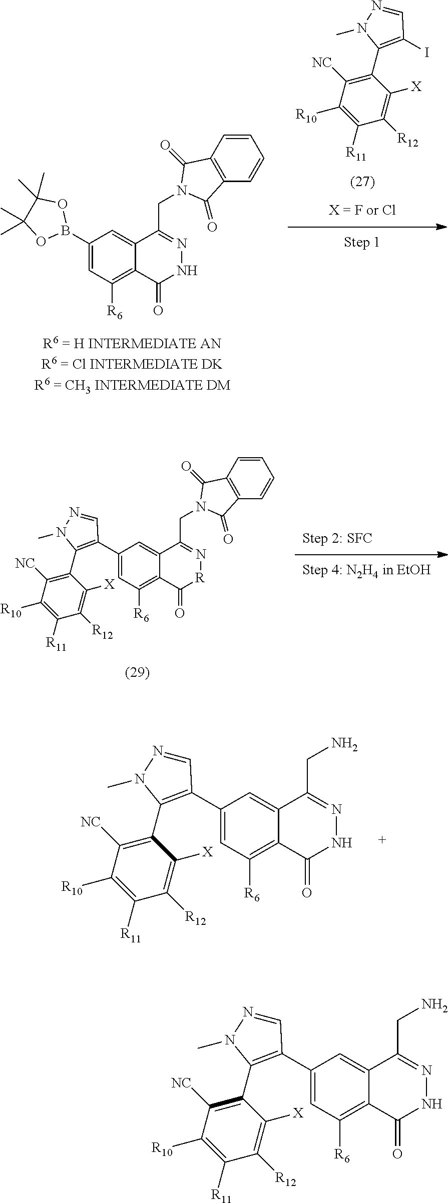

Step 1: A mixture of the appropriate aryl/heteroaryl-iodide 27 (390 μmol, 1.5 eq.), intermediate AN or DK or DM (260 μmol, 1.00 eq.), palladium catalyst (See Table 16-CM) (26.0 μmol, 0.10 eq.), base (See Table 16-CM) (0.521 μmol, 2.0-3.0 eq.) in solvent (1.2 mL, See Table 16-CM) was purged with nitrogen 3 times. The mixture was then stirred at 80° C. for 2 hours. After such time the mixture was purified using the methods described for CM 10A-1 or CM 10A-2 to give coupling product 29.

Step 2: The mixture of stereoisomers 29 was separated by SFC purification (DAICEL CHIRALPAK IC (250 mm×30 mm, 10 μm); mobile phase: [0.1% NH₃ water IPA]; B %: isocratic)

Step 2: To each of the separated isomers 29 in ethyl alcohol (1.0 mL) was added hydrazine hydrate (20 eq.). The mixture was stirred at 80° C. for 1 hour. After such time the mixture was concentrated in vacuo and the residue purified by prep-HPLC according to one of the purification methods (PM) 4-1 through 4-23.

### General Coupling Method CM-16-5

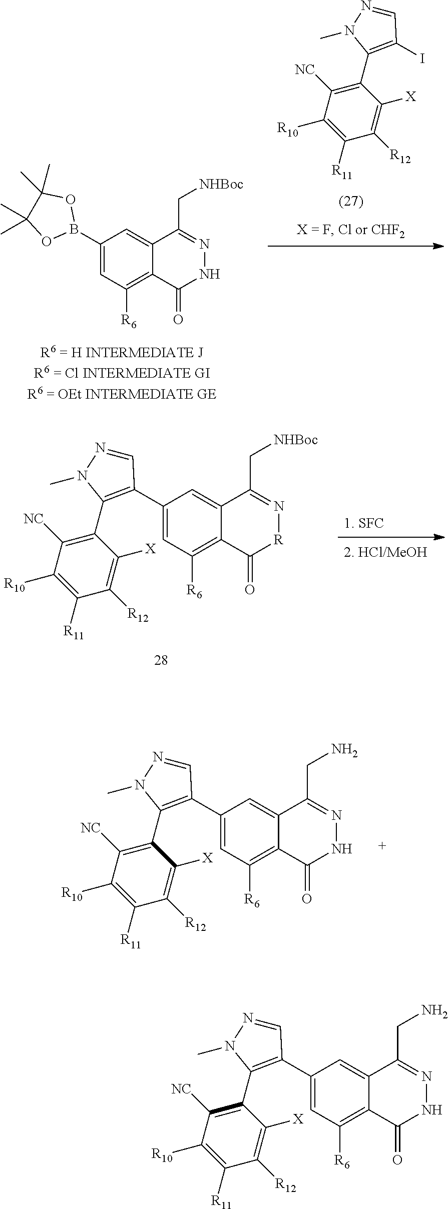

Step 1: A mixture of the appropriate aryl/heteroaryl-iodide 27 (390 μmol, 1.5 eq.), intermediate J or GI or GE (260 μmol, 1.00 eq.), palladium catalyst (See Table 16-CM) (26.0 μmol, 0.10 eq.), base (See Table 16-CM) (0.521 μmol, 2.0-3.0 eq.) in solvent (1.2 mL, See Table 16-CM) was purged with nitrogen 3 times. The mixture was then stirred at 80° C. for 2 hours. After such time the mixture was purified using the methods described for CM 10A-1 or CM 10A-2 to give coupling product 28.

Step 2: The mixture of stereoisomers 28 was separated by SFC purification (DAICEL CHIRALPAK IC (250 mm×30 mm×10 μm); mobile phase: [0.1% NH₃ water IPA]; B %: isocratic)

Step 3: To each of the separated isomers 28 in DCM (2.0 mL) was added TFA (0.4 mL). The mixture was stirred at 25° C. for 0.5 hour. After such time the mixture was concentrated in vacuo and the residue purified by prep-HPLC according to one of the purification methods (PM) 4-1 through 4-24.

TABLE 16-CM
GENRAL COUPLING CONDITIONS FOR CM-16-1,
CM-16-2, CM-16-2* CM-16-3, CM-16-4

| ID | catalyst | base | solvent |
|---|---|---|---|
| A | Pd(dtbpf)Cl₂ | NaHCO₃ | dioxane-water (5:1) |
| B | (Ad₂ n-BuP)-Pd G3 | NaHCO₃ | DMF |
| C | (Ad₂ n-BuP)-Pd G3 | K₃PO₄ | dioxane-water (5:1) |
| D | Pd(dppf)Cl₂ | K₂CO₃ | dioxane-water (5:1) |
| E | Pd(dppf)Cl₂*DCM | NaHCO₃ | dioxane-water (5:1) |
| F | (Ad₂ n-BuP)-Pd G3 | K₃PO₄ | DMF |
| G | (Ad₂ n-BuP)-Pd G3 | K₃PO₄ | DMAc |
| H | (Ad₂ n-BuP)-Pd G3 | K₃PO₄ | n-BuOH |
| I | Pd(dppf)Cl₂*DCM | NaHCO₃ | DMF |
| J | (Ad₂ n-BuP)-Pd G2 | CsF | dioxane-water (5:1) |
| K | (Ad₂ n-BuP)-Pd G3 | NaHCO₃ | dioxane-water (5:1) |
| L | cataXum-Pd G3 | NaHCO₃ | dioxane-water (5:1) |
| M | (Ad₂ n-BuP)-Pd G3 | Na₂CO₃ | dioxane-water (5:1) |
| N | Pd(dppf)Cl₂ | Cs₂CO₃ | dioxane-water (5:1) |
| O | Pd(dtbpf)Cl₂ | Na₂CO₃ | dioxane-water (5:1) |

Following the general coupling methods CM 16-1X, CM 16-2X, CM 16-2*X, CM-16-3X and CM 16-4X (where X is A through K from Table 16-CM) the Examples 16-9 to 16-138 were prepared as shown in Table 16. Odd example numbers were peak 1 and even numbers were peak 2 following SFC separation step.

TABLE 16

| Example | Structure | CM | PM | Compound Name and Characterization |
|---|---|---|---|---|
| 16-9 | 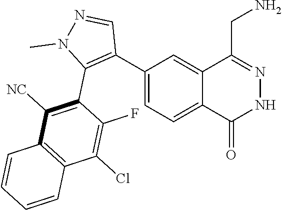 | 16-1A | 4-6 | (P)-2-[4-[4-(aminomethyl)-1-oxo-2H- phthalazin-6-yl]-2-methyl-pyrazol-3- yl]-4-chloro-3-fluoro-naphthalene-1- carbonitrile LCMS [M + 1] ⁺ 459.1. 1H NMR (400 MHz, MeOD) δ = 8.53 (d, J = 8.4 Hz, 1H), 8.27 (d, J = 8.0 Hz, 1H), 8.24 (s, 1H), 8.16 (d, J = 8.4 Hz, 1H), 8.02- 7.96 (m, 1H), 7.92 (d, J = 7.6 Hz, 1H), 7.90-7.87 (m, 1H), 7.52-7.42 (m, 1H), 4.50-4.32 (m, 2H), 3.89 (s, 3H) |
| 16-10 | 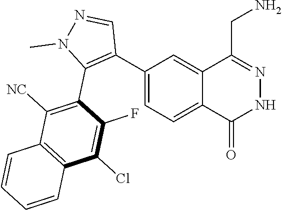 | 16-1A | 4-6 | (M)-2-[4-[4-(aminomethyl)-1-oxo-2H- phthalazin-6-yl]-2-methyl-pyrazol-3- yl]-4-chloro-3-fluoro-naphthalene-1- carbonitrile. LCMS [M + 1] ⁺ 459.1. 1H NMR (400 MHz, MeOD) δ = 8.53 (d, J = 8.4 Hz, 1H), 8.27 (d, J = 8.0 Hz, 1H), 8.24 (s, 1H), 8.16 (d, J = 8.4 Hz, 1H), 8.02- 7.96 (m, 1H), 7.92 (d, J = 7.6 Hz, 1H), 7.90-7.87 (m, 1H), 7.52-7.42 (m, 1H), 4.50-4.32 (m, 2H), 3.89 (s, 3H) |
| 16-11 | 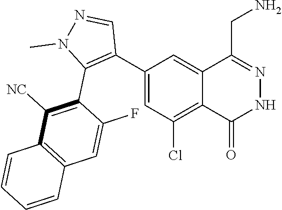 | 16-3B | 4-6 | (P)-2-(4-(4-(aminomethyl)-8-chloro- 1-oxo-1,2-dihydrophthalazin-6-yl)-1- methyl-1H-pyrazol-5-yl)-3-fluoro-1- naphthonitrile LC-MS [M + 1]⁺ = 459.1. ¹H NMR (400 MHz, MeOD) δ = 8.31 (d, J = 10.0 Hz, 1H), 8.26 (s, 1H), 8.24-8.15 (m, 2H), 7.88-7.80 (m, 2H), 7.67 (d, J = 1.6 Hz, 1H), 7.40 (d, J = 1.6 Hz, 1H), 4.42-4.17 (m, 2H), 3.86 (s, 3H) |
| 16-12 | 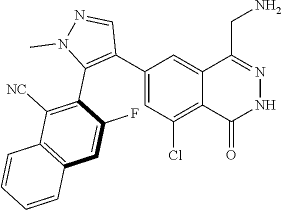 | 16-3B | 4-6 | (M)-(4-(4-(aminomethyl)-8-chloro-1- oxo-1,2-dihydrophthalazin-6-yl)-1- methyl-1H-pyrazol-5-yl)-3-fluoro-1- naphthonitrile LC-MS [M + 1]⁺ = 459.1. ¹H NMR (400 MHz, MeOD-d₄) δ = 8.30 (d, J = 10.0 Hz, 1H), 8.23 (s, 1H), 8.23-8.15 (m, 2H), 7.86-7.79 (m, 2H), 7.61 (d, J = 1.6 Hz, 1H), 7.51 (d, J = 1.6 Hz, 1H), 3.86 (s, 3H), 3.74-3.56 (m, 2H) |
| 16-13 | 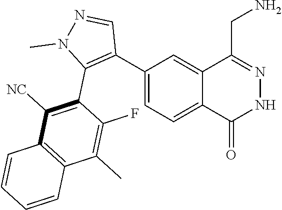 | 16-1C | 4-6 | (P)-2-[4-[4-(aminomethyl)-1-oxo-2H- phthalazin-6-yl]-2-methyl-pyrazol-3- yl]-3-fluoro-4-methyl-naphthalene-1- carbonitrile. LCMS [M + 1] ⁺ 439.2. ¹H NMR (400 MHz, CD₃OD) δ = 8.34 (d, J = 8.4 Hz, 1H), 8.23 (s, 1H), 8.18 (d, J = 8.0 Hz, 1H), 8.13 (d, J = 8.4 Hz, 1H), 7.91-7.84 (m, 2H), 7.84-7.78 (m, 1H), 7.45 (dd, J = 1.6, 8.4 Hz, 1H), 4.51- 4.31 (m, 2H), 3.85 (s, 3H), 2.80 (d, J = 2.4 Hz, 3H) |
| 16-14 | 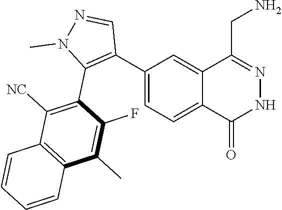 | 16-1C | 4-6 | (M)-2-[4-[4-(aminomethyl)-1-oxo-2H- phthalazin-6-yl]-2-methyl-pyrazol-3- yl]-3-fluoro-4-methyl-naphthalene-1- carbonitrile. LCMS [M + 1] ⁺ 439.2. ¹H NMR (400 MHz, CD₃OD) δ = 8.34 (br d, J = 8.4 Hz, 1H), 8.23 (s, 1H), 8.18 (br d, J = 8.4 Hz, 1H), 8.13 (br d, J = 8.0 Hz, 1H), 7.92-7.84 (m, 2H), 7.84-7.77 (m, 1H), 7.49-7.42 (m, 1H), 4.51- 4.31 (m, 2H), 3.85 (s, 3H), 2.80 (d, J = 2.4 Hz, 3H) |
| 16-15 | 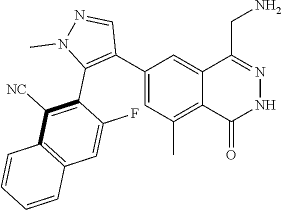 | 16-4B | 4-6 | (P)-2-[4-[4-(aminomethyl)-8-methyl- 1-oxo-2H-phthalazin-6-yl]-2-methyl- pyrazol-3-yl]-3-fluoro-naphthalene-1- carbonitrile LC-MS [M + 1]⁺ = 439.2. ¹H NMR (400 MHz, MeOD) δ = 8.30 (d, J = 10.0 Hz, 1H), 8.22-8.16 (m, 3H), 7.87- 7.79 (m, 2H), 7.51 (d, J = 1,2 Hz, 1H), 7.27 (s, 1H), 4.38-4.10 (m, 2H), 3.86 (s, 3H), 2.63 (s, 3H) |
| 16-16 | 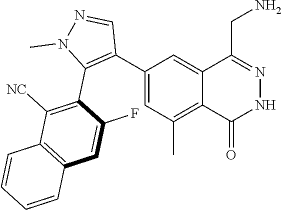 | 16-4B | 4-6 | (M)-2-[4-[4-(aminomethyl)-8-methyl- 1-oxo-2H-phthalazin-6-yl]-2-methyl- pyrazol-3-yl]-3-fluoro-naphthalene-1- carbonitrile LC-MS [M + 1]⁺ = 439.2. ¹H NMR (400 MHz, MeOD-d₄) δ = 8.29 (d, J = 10.0 Hz, 1H), 8.22-8.16 (m, 3H), 7.87- 7.80 (m, 2H), 7.52 (s, 1H), 7.27 (s, 1H), 4.34-4.11 (m, 2H), 3.86 (s, 3H), 2.63 (s, 3H). |
| 16-17 | 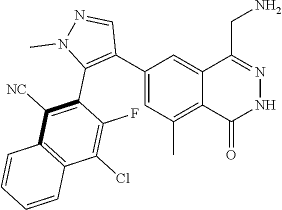 | 16-3A | 4-18 | (P)-2-[4-[4-(aminomethyl)-8-methyl- 1-oxo-2H-phthalazin-6-yl]-2-methyl- pyrazol-3-yl]-4-chloro-3-fluoro- naphthalene-1-carbonitrile. LCMS [M + 1] ⁺ 473.2. ¹H NMR (400 MHz, MeOD) δ = 8.52 (d, J = 8.4 Hz, 1H), 8.28 (d, J = 8.4 Hz, 1H), 8.18 (s, 1H), 8.03-7,96 (m, 1H), 7.94-7.88 (m, 1H), 7.45 (s, 1H), 7.37 (s, 1H), 3.97-3.79 (m, 5H), 2.69 (s, 3H) |
| 16-18 | 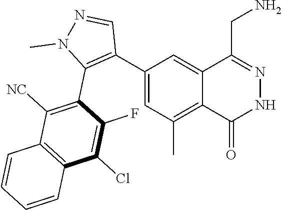 | 16-3A | 4-18 | (M)-2-[4-[4-(aminomethyl)-8-methyl- 1-oxo-2H-phthalazin-6-yl]-2-methyl- pyrazol-3-yl]-4-chloro-3-fluoro- naphthalene-1-carbonitrile. LCMS [M + 1] ⁺ 473.2. ¹H NMR (400 MHz, MeOD) δ = 8.52 (br d, J = 8.4 Hz, 1H), 8.28 (d, J = 8.0 Hz, 1H), 8.19 (s, 1H), 8.02-7.96 (m, 1H), 7.95- 7.89 (m, 1H), 7.46 (s, 1H), 7.37 (s, 1H), 3.98-3.81 (m, 5H), 2.70 (s, 3H) |
| 16-19 | 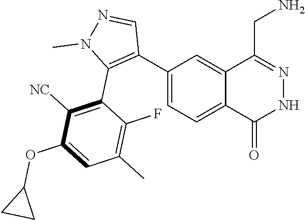 | 16-2C | 4-6 | (P)-2-[4-[4-(aminomethyl)-1-oxo-2H- phthalazin-6-yl]-2-methyl-pyrazol-3- yl]-6-(cyclopropoxy)-3-fluoro-4- methyl-benzonitrile. LC-MS [M + 1]⁺ = 445.3. ¹H NMR (400 MHz, DMSO-d₆) δ = 12.86 (s, 1H), 8.61 (br s, 3H), 8.37 (s, 1H), 8.13 (d, J = 8.4 Hz, 1H), 7.75 (d, J = 6.0 Hz, 1H), 7.73 (d, J = 1.2 Hz, 1H), 7.51 (dd, J = 1.2, 8.4 Hz, 1H), 4.37-4.18 (m, 2H), 4.17-4.10 (m, 1H), 4.02 (br s, 3H), 2.45 (d, J = 1.6 Hz, 3H), 0.93- 0.83 (m, 2H), 0.83-0.72 (m, 2H) |
| 16-20 | 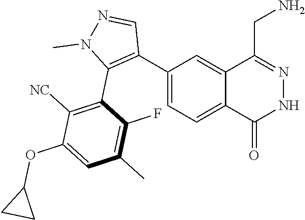 | 16-2C | 4-6 | (M)-2-[4-[4-(aminomethyl)-1-oxo-2H- phthalazin-6-yl]-2-methyl-pyrazol-3- yl]-6-(cyclopropoxy)-3-fluoro-4- methyl-benzonitrile. LC-MS [M + 1]⁺ = 445.3. ¹H NMR (400 MHz, MeOD) δ = 8.23 (br d, J = 8.4 Hz, 1H), 8.18-8.13 (m, 1H), 7.90- 7.81 (m, 1H), 7.65 (br d, J = 6.4 Hz, 1H), 7.49-7.44 (m, 1H), 4.56-4.47 (m, 2H), 4.06-3.98 (m, 1H), 3.83- 3.76 (m, 3H), 2.49 (br d, J = 2.0 Hz, 3H), 0.95-0.86 (m, 2H), 0.82-0.76 (m, 2H) |
| 16-21 | 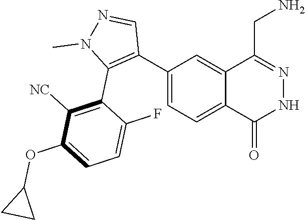 | 16- 2*D | 4-20 | (P)-2-[4-[4-(aminomethyl)-1-oxo-2H- phthalazin-6-yl]-2-methyl-pyrazol-3- yl]-6-(cyclopropoxy)-3-fluoro- benzonitrile. LCMS [ESI, M + 1]⁺ = 431.2. ¹H NMR (400 MHz, MeOD-d₆) δ = 7.72 (d, J = 8.4 Hz, 1H), 7.65 (s, 1H), 7.32 (s, 1H), 7.30-7.18 (m, 2H), 7.03-6.95 (m, 1H), 4.07-3.88 (m, 2H), 3.54 (td, J = 3.2, 5.6 Hz, 1H), 3.31 (s, 3H), 0.41 (br dd, J = 3.2, 5.2 Hz, 2H), 0.31 (br dd, J = 2.8, 5.2 Hz, 2H). |
| 16-22 | 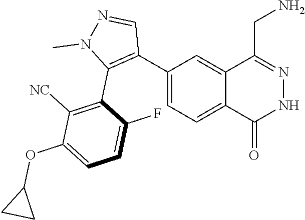 | 16- 2*D | 4-20 | (M)-2-[4-[4-(aminomethyl)-1-oxo- 2H-phthalazin-6-yl]-2-methyl- pyrazol-3-yl]-6-(cyclopropoxy)-3- fluoro-benzonitrile. LCMS [ESI, M +1] ⁺ = 431.2. ¹H NMR (400 MHz, MeOD-d6) δ = 8.21 (d, J = 8.4 Hz, 1H), 8.16 (s, 1H), 7.81 (d, J = 1.2 Hz, 1H), 7.80-7.69 (m, 2H), 7.49 (dd, J = 1.2, 8.4 Hz, 1H), 4.56-4.41 (m, 2H), 4.05 (tt, J = 2.8, 6.0 Hz, 1H), 3.81 (s, 3H), 0.91 (br dd, J = 3.6, 5.6 Hz, 2H), 0.86-0.77 (m, 2H). |
| 16-23 | 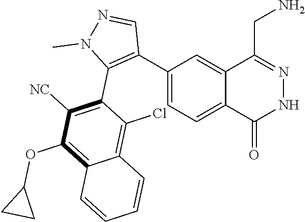 | 12- 2*K | 4-13 | (P)-3-[4-[4-(aminomethyl)-1-oxo-2H- phthalazin-6-yl]-2-methyl-pyrazol-3- yl]-4-chloro-1-(cyclopropoxy) naphthalene-2-carbonitrile. LCMS [M + 1] ⁺ = 497.1. ¹H NMR (400 MHz, MeOD-d₄) δ = 8.46 (d, J = 8.5 Hz, 1H), 8.40 (d, J = 8.4 Hz, 1H), 8.25 (s, 1H), 8.15 (d, J = 8.4 Hz, 1H), 8.02- 7.94 (m, 1H), 7.92-7.81 (m, 2H), 7.49-7.39 (m, 1H), 4.64 (tt, J = 2.9, 6.0 Hz, 1H), 4.51-4.33 (m, 2H), 3.82 (s, 3H), 1.07-0.98 (m, 1H), 0.98- 0.89 (m, 1H), 0.86-0.78 (m, 1H), 0.78-0.68 (m, 1H). |
| 16-24 | 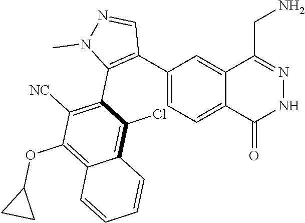 | 12- 2*K | 4-13 | (M)-3-[4-[4-(aminomethyl)-1-oxo- 2H-phthalazin-6-yl]-2-methyl- pyrazol-3-yl]-4-chloro-1- (cyclopropoxy)naphthalene-2- carbonitrile. LCMS [M + 1] ⁺ = 497.1. ¹H NMR (400 MHz, MeOD-d₄) δ = 8.45 (d, J = 8.4 Hz, 1H), 8.40 (d, J = 8.4 Hz, 1H), 8.25 (s, 1H), 8.15 (d, J = 8.4 Hz, 1H), 8.01-7.94 (m, 1H), 7.92-7.82 (m, 2H), 7.48 (d, J = 8.4 Hz, 1H), 4.64 (tt, J = 2.8, 6.0 Hz, 1H), 4.52-4.34 (m, 2H), 3.82 (s, 3H), 1.05-0.98 (m, 1H), 0.97-0.89 (m, 1H), 0.87-0.78 (m, |
| | | | | 1H), 0.78-0.66 (m, 1H). |
| 16-25 | 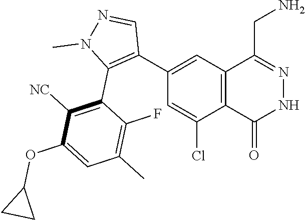 | 16-4A | 4-6 | (P)-2-[4-[4-(aminomethyl)-8-chloro- 1-oxo-2H-phthalazin-6-yl]-2-methyl- pyrazol-3-yl]-6-(cyclopropoxy)-3- fluoro-4-methyl-benzonitrile LCMS [M + 1] ⁺ = 479.1. ¹H NMR (400 MHz, MeOD-d₄) δ ppm 8.20 (s, 1 H) 7.70 (d, J = 6.0 Hz, 1 H) 7.65 (s, 1 H) 7.36 (d, J = 1.6 Hz, 1 H) 4.30-4.47 (m, 2 H) 4.07 (tt, J = 6.0, 3.2 Hz, 1 H) 3.81 (s, 3 H) 2.51 (d, J = 2.0 Hz, 3 H) 0.87-0.98 (m, 2 H) 0.72-0.85 (m, 2 H) |
| 16-26 | 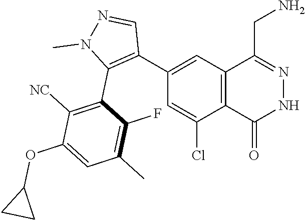 | 16-4A | 4-6 | (M)-2-[4-[4-(aminomethyl)-8-chloro- 1-oxo-2H-phthalazin-6-yl]-2-methyl- pyrazol-3-yl]-6-(cyclopropoxy)-3- fluoro-4-methyl-benzonitrile LCMS [M + 1] ⁺ = 479.1. ¹H NMR (400 MHz, MeOD-d₄) δ ppm 8.20 (s, 1 H) 7.70 (d, J = 6.0 Hz, 1 H) 7.65 (d, J = 1.6 Hz, 1 H) 7,36 (d, J = 1.6 Hz, 1 H) 4.30-4.48 (m, 2 H) 4.07 (dt, J = 6.0, 3.2 Hz, 1 H) 3.81 (s, 3 H) 2.51 (d, J = 2.0 Hz, 3 H) 0.86-0.94 (m, 2 H) 0.73- 0.84 (m, 2 H) |
| 16-27 | 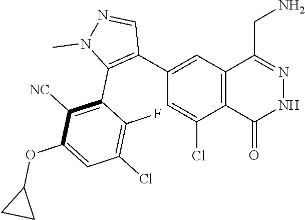 | 16-4E | 4-1 | (P)-2-[4-[4-(aminomethyl)-8-chloro- 1-oxo-2H-phthalazin-6-yl]-2-methyl- pyrazol-3-yl]-4-chloro-6- (cyclopropoxy)-3-fluoro-benzonitrile. LCMS [ESI, M + 1]⁺ = 499.2. ¹H NMR (400 MHz, MeOD) δ = 8.21 (s, 1H), 7.96 (d, J = 6.0 Hz, 1H), 7.71 (d, J = 1.2 Hz, 1H), 7.38 (d, J = 1.2 Hz, 1H), 4.53-4.35 (m, 2H), 4.12 (tt, J = 2.4, 5.9 Hz, 1H), 3.84 (s, 3H), 2.66 (s, 1H), 1.05-0.75 (m, 4H). |
| 16-28 | 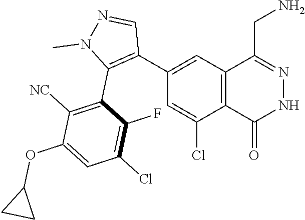 | 16-4E | 4-1 | (M)-2-[4-[4-(aminomethyl)-8-chloro- 1-oxo-2H-phthalazin-6-yl]-2-methyl- pyrazol-3-yl]-4-chloro-6- (cyclopropoxy)-3-fluoro-benzonitrile. LCMS [ESI, M + 1]⁺ = 499.2. ¹H NMR (400 MHz, MeOD) δ = 8.20 (s, 1H), 7.96 (d, J = 6.0 Hz, 1H), 7.71 (d, J = 1.6 Hz, 1H), 7.38 (d, J = 1.2 Hz, 1H), 4.54-4.37 (m, 2H), 4.12 (tt, J = 3.2, 5.9 Hz, 1H), 3.84 (s, 3H), 1.01-0.76 (m, 4H). |
| 16-29 | 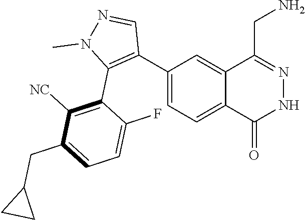 | 16-2D | 4-13 | (P)-2-(4-(4-(aminomethyl)-1-oxo-1,2- dihydrophthalazin-6-yl)-1-methyl-1H- pyrazol-5-yl)-6-(cyclopropylmethyl)- 3-fluorobenzonitrile LCMS [M + 1] ⁺: 429.2. ¹H NMR (400 MHz, CD₃OD): δ = 8.23-8.18 (m, 2H), 7.85-7.80 (m, 2H), 7.70 (t, J = 8.8 Hz, 1H), 7.46 (d, J = 8.4 Hz, 1H), 4.53-4.39 (m, 2H), 3.83 (s, 3H), 2.83- 2.69 (m, 2H), 1.05-1.01 (m, 1H), 0.58- 0.55 (m, 2H), 0.25-0.20 (m, 2H). |
| 16-30 | 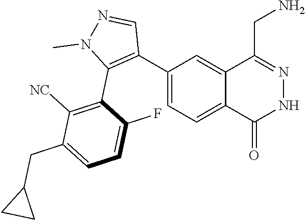 | 16-2D | 4-13 | (M)-2-(4-(4-(aminomethyl)-1-oxo-1,2- dihydrophthalazin-6-yl)-1-methyl-1H- pyrazol-5-yl)-6-(cyclopropylmethyl)- 3-fluorobenzonitrile LCMS [M + 1] ⁺: 429.2. ¹H NMR (400 MHz, CD₃OD): δ = 8.23-8.18 (m, 2H), 7.85-7.80 (m, 2H), 7.70 (t, J = 8.8 Hz, 1H), 7.47 (d, J = 8.4 Hz, 1H), 4.53-4.39 (m, 2H), 3.83 (s, 3H), 2.83- 2.69 (m, 2H), 1.04-1.01 (m, 1H), 0.55- 0.49 (m, 2H), 0.27-0.20 (m, 2H). |
| 16-31 | 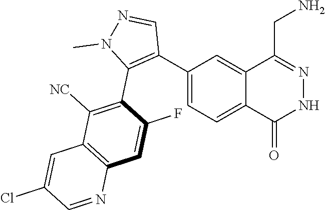 | 16- 2*A | 4-21 | (M)-6-[4-[4-(aminomethyl)-1-oxo-2H- phthalazin-6-yl]-2-methyl-pyrazol-3- yl]-3-chloro-7-fluoro-quinoline-5- carbonitrile. LCMS [ESI, M + 1] ⁺: 460.1. ¹H NMR (400 MHz, DMSO-d₆) δ = 12.86 (s, 1H), 9.25 (d, J = 2.0 Hz, 1H), 8.60- 8.54 (m, 2H), 8.45-8.37 (m, 4H), 8.05 (d, J = 8.4 Hz, 1H), 7,90 (s, 1H), 7.44 (br d, J = 8.4 Hz, 1H), 4.37-4.25 (m, 2H), 3.83 (s, 3H). |
| 16-32 | 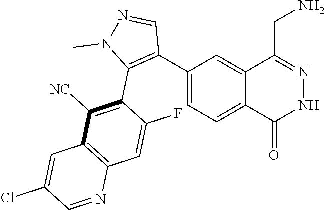 | 16- 2*A | 4-21 | (P)-6-[4-[4-(aminomethyl)-1-oxo-2H- phthalazin-6-yl]-2-methyl-pyrazol-3- yl]-3-chloro-7-fluoro-quinoline-5- carbonitrile. LCMS [ESI, M + 1] ⁺: 460.1. ¹H NMR (400 MHz, DMSO-d₆) δ = 12.86 (s, 1H), 9.25 (d, J = 2.4 Hz, 1H), 8.62- 8.54 (m, 2H), 8.43 (s, 1H), 8.38 (br s, 2H), 8.05 (d, J = 8.4 Hz, 1H), 7.89 (d, J = 1.2 Hz, 1H), 7.44 (dd, J = 1.2, 8.4 Hz, 1H), 4.39-4.26 (m, 2H), 3.83 (s, 3H) |
| 16-33 | 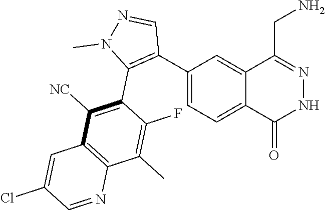 | 16-1F | 4-6 | (P)-6-14-[4-(aminomethyl)-1-oxo-2H- phthalazin-6-yl]-2-methyl-pyrazol-3- yl]-3-chloro-7-fluoro-8-methyl- quinoline-5-carbonitrile. LCMS [M + 1] ⁺: 474.1. 1H NMR (400 MHz, MeOD-d₄) δ = 9.10 (d, J = 2.4 Hz, 1H), 8.51 (d, J = 2.4 Hz, 1H), 8.21 (s, 1H), 8.16 (d, J = 8.4 Hz, 1H), 7.78 (d, J = 1.2 Hz, 1H), 7.53 (dd, J = 1.6, 8.4 Hz, 1H), 4.03 (q, J = 16.0 Hz, 2H), 3.86 (s, 3H), 3.60 (q, J = 7.2 Hz, 1H), 2.86 (d, J = 2.8 Hz, 3H). |
| 16-34 | 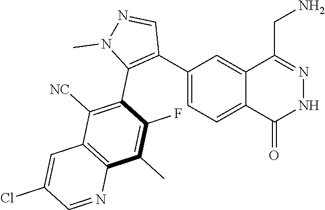 | 16-1F | 4-6 | (M)-6-[4-[4-(aminomethyl)-1-oxo-2H- phthalazin-6-yl]-2-methyl-pyrazol-3- yl]-3-chloro-7-fluoro-8-methyl- quinoline-5-carbonitrile. LCMS [M + 1] ⁺: 474.1. ¹H NMR (400 MHz, MeOD-d₄) δ = 9.11 (d, J = 2.4 Hz, 1H), 8.51 (d, J = 2.4 Hz, 1H), 8.22 (s, 1H), 8.16 (d, J = 8.4 Hz, 1H), 7.79 (s, 1H), 7.53 (d, J = 8.4 Hz, 1H), 4.20- 4.00 (m, 2H), 3.87 (s, 3H), 2.86 (d, J = 2.8 Hz, 3H). |
| 16-35 | 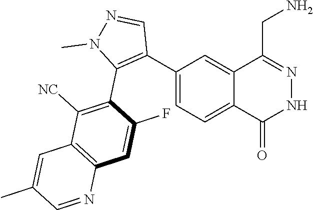 | 16- 2*A | 4-21 | (M)-6-[4-[4-(aminomethyl)-1-oxo-2H- phthalazin-6-yl]-2-methyl-pyrazol-3- yl]-7-fluoro-3-methyl-quinoline-5- carbonitrile. LCMS [ESI, M + 1] ⁺: 440.2. ¹H NMR (400 MHz, MeOD-d₄) δ = 9.14 (br s, 1H), 8.52 (br s, 1H), 8.36 (br d, J = 8.8 Hz, 1H), 8.25 (s, 1H), 8.14 (br d, J = 7.2 Hz, 1H), 7.88 (s, 1H), 7.46 (br d, J = 7.2 Hz, 1H), 4.55-4.34 (m, 2H), 3.89 (s, 3H), 2.67 (s, 3H). |
| 16-36 | 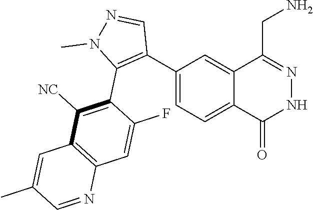 | 16- 2*A | 4-21 | (P)-6-[4-[4-(aminomethyl)-1-oxo-2H- phthalazin-6-yl]-2-methyl-pyrazol-3- yl]-7-fluoro-3-methyl-quinoline-5- carbonitrile. LCMS [ESI, M + 1] ⁺: 440.2. ¹H NMR (400 MHz, MeOD-d₄) δ = 9.13 (s, 1H), 8.49 (s, 1H), 8.35 (br d, J = 9.6 Hz, 1H), 8.25 (s, 1H), 8.14 (br d, J = 8.0 Hz, 1H), 7.88 (s, 1H), 7.46 (br d, J = 8.0 Hz, 1H), 4,56-4.37 (m, 2H), 3,88 (s, 3H), 2.66 (s, 3H). |
| 16-37 | 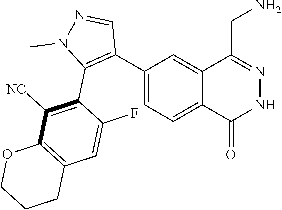 | 16-1C | 4-13 | (P)-7-[4-[4-(aminomethyl)-1-oxo-2H- phthalazin-6-yl]-2-methyl-pyrazol-3- yl]-6-fluoro-chromane-8-carbonitrile. LCMS [M + 1] ⁺ = 431.0. ¹H NMR (400 MHz, MeOD-d₄) δ = 8.22 (d, J = 8.4 Hz, 1H), 8.15 (s, 1H), 7.83 (d, J = 1.2 Hz, 1H), 7.52 (dd, J = 1.6, 8.4 Hz, 1H), 7.47 (d, J = 9.4 Hz, 1H), 4.57- 4.43 (m, 2H), 4.55-4.45 (m, 2H), 3.81 (s, 3H), 3.04-2.91 (m, 2H), 2.12- 2.07 (m, 2H). |
| 16-38 | 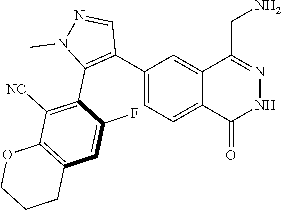 | 16-1C | 4-13 | (M)-7-[4-[4-(aminomethyl)-1-oxo-2H- phthalazin-6-yl]-2-methyl-pyrazol-3- yl]-6-fluoro-chromane-8-carbonitrile. LCMS [M + 1] ⁺ = 431.1. ¹H NMR (400 MHz, MeOD-d₄) δ = 8.23 (d, J = 8.4 Hz, 1H), 8.15 (s, 1H), 7,84 (d, J = 1.2 Hz, 1H), 7.52 (dd, J = 1.2, 8.4 Hz, 1H), 7.47 (d, J = 9.2 Hz, 1H), 4.59- 4.42 (m, 2H), 4.54-4.44 (m, 2H), 3.81 (s, 3H), 3.04-2.91 (m, 2H), 2.20- 1.99 (m, 2H). |
| 16-39 | 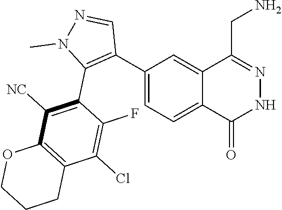 | 16-2C | 4-22 | (P)-7-(4-(4-(aminomethyl)-1-oxo-1,2- dihydrophthalazin-6-yl)-1-methyl-1H- pyrazol-5-yl)-5-chloro-6- fluorochroman-8-carbonitrile. LCMS [M + 1] ⁺: 465.3. ¹H NMR (400 MHz, DMSO-d₆) δ = 12.88 (s, 1H), 8.60-8.47 (m, 3H), 8.37 (s, 1H), 8.15 (d, J = 8.4 Hz, 1H), 7.83 (d, J = 1.2 Hz, 1H), 7.51 (dd, J = 8.4, 1.6 Hz, 1H), 4.43-4.27 (m, 4H), 3.77 (s, 3H), 3.04-2.82 (m, 2H), 2.17-2.00 (m, 2H) |
| 16-40 | 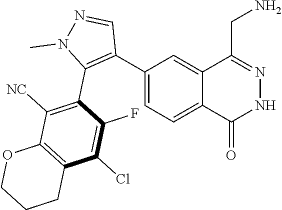 | 16-2C | 4-22 | (M)-7-(4-(4-(aminomethyl)-1-oxo-1,2- dihydrophthalazin-6-yl)-1-methyl-1H- pyrazol-5-yl)-5-chloro-6- fluorochroman-8-carbonitrile LCMS [M + 1] ⁺: 465.3. ¹H NMR (400 MHz, DMSO-d₆) δ = 12.89 (s, 1H), 8.53- 8.36 (m, 3H), 8.36-8.33 (m, 1H), 8.15 (d, J = 8.8 Hz, 1H), 7.84 (s, 1H), 7.53- 7.47 (m, 1H), 4.43-4.30 (m, 4H), 3.77 (s, 3H), 2,97-2.84 (m, 2H), 2.13-2.03 (m, 2H) |
| 16-41 | 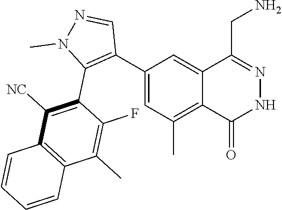 | 16-3C | 4-18 | (P)-2-[4-[4-(aminomethyl)-8-methyl- 1-oxo-2H-phthalazin-6-yl]-2-methyl- pyrazol-3-yl]-3-fluoro-4-methyl- naphthalene-1-carbonitrile. LCMS [M + 1] ⁺ 453.2. ¹H NMR (400 MHz, MeOD) δ = 8.52 (s, 1H), 8.35 (d, J = 8.4 Hz, 1H), 8.24-8.16 (m, 2H), 7.91-7,86 (m, 1H), 7.84-7.79 (m, 1H), 7,48 (s, 1H), 7.30 (s, 1H), 4.25-4.15 (m, 1H), 4.08-3.98 (m, 1H), 3.85 (s, 3H), 2.81 (d, J = 2.5 Hz, 3H), 2.63 (s, 3H) |
| 16-42 | 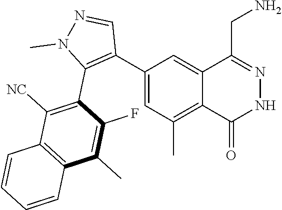 | 16-3C | 4-18 | (M)-2-[4-[4-(aminomethyl)-8-methyl- 1-oxo-2H-phthalazin-6-yl]-2-methyl- pyrazol-3-yl]-3-fluoro-4-methyl- naphthalene-1-carbonitrile. LCMS [M + 1] ⁺ 453.2. ¹H NMR (400 MHz, MeOD) δ = 8.54 (s, 1H), 8.35 (d, J = 8.4 Hz, 1H), 8.24-8.18 (m, 2H), 7.91-7.85 (m, 1H), 7.84-7.79 (m, 1H), 7.41 (s, 1H), 7.37 (s, 1H), 3.99-3.91 (m, 1H), 3.85 (s, 3H), 3.83- 3.74 (m, 1H), 2.81 (d, J = 2.5 Hz, 3H), 2.67 (s, 3H) |
| 16-43 | 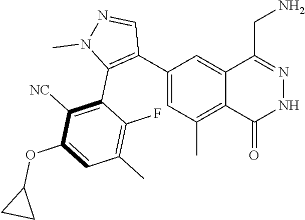 | 16-4A | 4-6 | (P)-2-(4-(4-(aminomethyl)-8-methyl- 1-oxo-1,2-dihydrophthalazin-6-yl)-1- methyl-1H-pyrazol-5-yl)-6- cyclopropoxy-3-fluoro-4- methylbenzonitrile. LCMS [M + 1]⁺: 459.2. ¹H NMR (400 MHz, CD₃OD) δ = 8.13 (s, 1H), 7.66 (d, J = 6.0 Hz, 1H), 7.54 (s, 1H), 7.26 (s, 1H), 4.45-4.28 (m, 2H), 4.09- 4.00 (m, 1H), 3.80 (s, 3H), 2.74 (s, 3H), 2.50 (d, J = 1.6 Hz, 3H), 0.94- 0.88 (m, 2H), 0.82-0.76 (br d, J = 2.8 Hz, 2H) |
| 16-44 | 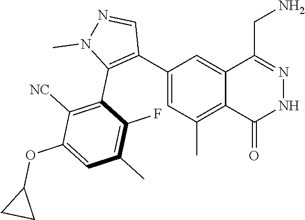 | 16-4A | 4-6 | (M)-2-(4-(4-(aminomethyl)-8-methyl- 1-oxo-1,2-dihydrophthalazin-6-yl)-1- methyl-1H-pyrazol-5-yl)-6- cyclopropoxy-3-fluoro-4- methylbenzonitrile. LCMS [M + 1]⁺: 459.2. ¹H NMR (400 MHz, CD₃OD) δ = 8.15 (s, 1H), 7.68 (d, J = 6.0 Hz, 1H), 7.57 (br s, 1H), 7.28 (s, 1H), 4.48-4.30 (m, 2H), 4.10- 4.03 (m, 1H), 3.82 (s, 3H), 2.76 (s, 3H), 2.52 (d, J = 1.6 Hz, 3H), 0.97- 0.89 (m, 2H), 0.86-0.76 (m, 2H) |
| 16-45 | 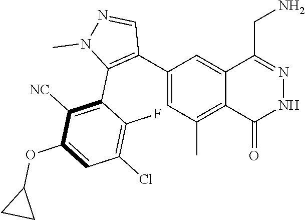 | 16-4A | 46 | (P)-2-[4-[4-(aminomethyl)-8-methyl- 1-oxo-2H-phthalazin-6-yl]-2-methyl- pyrazol-3-yl]-4-chloro-6- (cyclopropoxy)-3-fluoro-benzonitrile LCMS [M + 1]⁺: 479.2. ¹H NMR (400 MHz, CD₃OD) δ = 8.13 (s, 1H), 7.93 (d, J = 6.0 Hz, 1H), 7.57 (br s, 1H), 7.25 (s, 1H), 4.48-4.33 (m, 2H), 4.10 (dt, J = 2.9, 5.8 Hz, 1H), 3.83 (s, 3H), 2.76 (s, 3H), 0.98-0.90 (m, 2H), 0.86- 0.79 (m, 2H) |
| 16-46 | 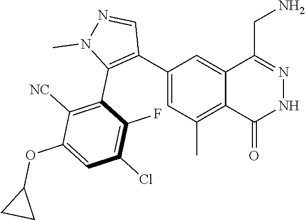 | 16-4A | 4-6 | (M)-2-[4-[4-(aminomethyl)-8-methyl- 1-oxo-2H-phthalazin-6-yl]-2-methyl- pyrazol-3-yl]-4-chloro-6- (cyclopropoxy)-3-fluoro-benzonitrile LCMS [M + 1]⁺: 479.2. ¹H NMR (400 MHz, CD₃OD) δ = 8.13 (s, 1H), 7.93 (d, J = 6.0 Hz, 1H), 7.56 (br s, 1H), 7.25 (s, 1H), 4.49-4.33 (m, 2H), 4.10 (br s, 1H), 3.83 (s, 3H), 2.76 (s, 3H), 1.00-0.91 (m, 2H), 0.87-0.76 (m, 2H) |
| 16-47 | 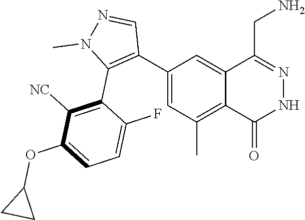 | 16-4E | 4-18 | (P)-2-[4-[4-(aminomethyl)-8-methyl- 1-oxo-2H-phthalazin-6-yl]-2-methyl- pyrazol-3-yl]-6-(cyclopropoxy)-3- fluoro-benzonitrile. LCMS [ESI, M + 1]⁺ = 445.2. ¹H NMR (400 MHz, CDCl3) δ = 9.44 (s, 1H), 7.96 (s, 1H), 7.93-7.88 (m, 2H), 7.81- 7.72 (m, 2H), 7.60-7.45 (m,2H), 7.39-7.32 (m, 2H), 4.95-4.63 (m, 2H), 3,95-3.89 (m, 1H), 3.86 (s, 3H), 2.82 (s, 3H), 0.99-0.86 (m, 4H) |
| 16-48 | 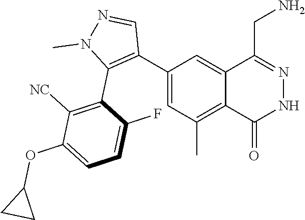 | 16-4E | 4-18 | (M)-2-[4-[4-(aminomethyl)-8-methyl- 1-oxo-2H-phthalazin-6-yl]-2-methyl- pyrazol-3-yl]-6-(cyclopropoxy)-3- fluoro-benzonitrile. LCMS [ESI, M + 1]⁺ = 445.2. ¹H NMR (400 MHz, MeOD-d4) δ = 8.54 (s, 1H), 8.12 (s, 1H), 7.84-7.68 (m, 2H), 7.47 (s, 1H), 7.33 (s, 1H), 4.22-4.09 (m, 2H), 4.08-4.02 (m, 1H), 3.81 (s, 3H), 2.76 (s, 3H), 1.00-0.75 (m, 4H) |
| 16-49 | 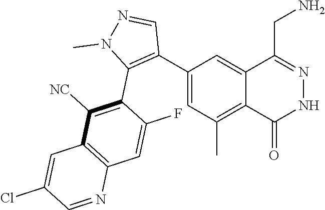 | 16-4E | 4-6 | (P)-6-[4-[4-(aminomethyl)-8-methyl- 1-oxo-2H-phthalazin-6-yl]-2-methyl- pyrazol-3-yl]-3-chloro-7-fluoro- quinoline-5-carbonitrile. LCMS [ESI, M + 1] ⁺: 474.1. ¹H NMR (400 MHz, MeOD) δ = 9.12 (d, J = 2.0 Hz, 1H), 8.57 (d, J = 1.2 Hz, 1H), 8.40 (d, J = 10.0 Hz, 1H), 8.20 (s, 1H), 7.51 (s, 1H), 7.30 (s, 1H), 4.36-4.28 (m, 1H), 4.26-4.17 (m, 1H), 3.88 (s, 3H), 2.67 (s, 3H). |
| 16-50 | 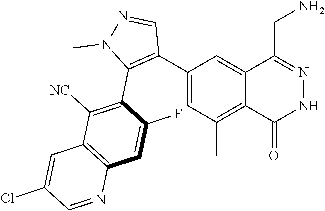 | 16-4E | 4-6 | (M)-6-[4-[4-(aminomethyl)-8-methyl- 1-oxo-2H-phthalazin-6-yl]-2-methyl- pyrazol-3-yl]-3-chloro-7-fluoro- quinoline-5-carbonitrile. LCMS [ESI, M + 1] ⁺: 474.1. ¹H NMR (400 MHz, MeOD) δ = 9.12 (s, 1H), 8.57 (d, J = 0.4 Hz, 1H), 8.40 (d, J = 9.6 Hz, 1H), 8.20 (s, 1H), 7.51 (s, 1H), 7.30 (s, 1H), 4.39-4.27 (m, 1H), 4.27- 4.15 (m, 1H), 3.88 (s, 3H), 2.67 (s, 3H) |
| 16-51 | 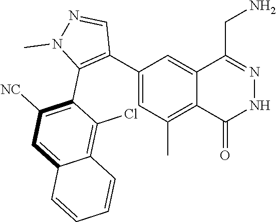 | 16-4G | 4-6 | (P)-3-[4-[4-(aminomethyl)-8-methyl- 1-oxo-2H-phthalazin-6-yl]-2-methyl- pyrazol-3-yl]-4-chloro-naphthalene-2- carbonitrile. LCMS [M + 1] ⁺ 455.1. ¹H NMR (400 MHz, CD₃OD) δ = 8.65 (s, 1H), 8.52 (d, J = 8.4 Hz, 1H), 8.26-8.19 (m, 2H), 8.00 (ddd, J = 1,2, 7.1, 8.4 Hz, 1H), 7.95-7,88 (m, 1H), 7.52 (d, J = 1.2 Hz, 1H), 7.27 (d, J = 0.4 Hz, 1H), 4.28-4.15 (m, 2H), 3.79 (s, 3H), 2.61 (s, 3H) |
| 16-52 | 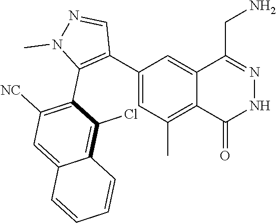 | 16-4G | 4-6 | (M)-3-[4-[4-(aminomethyl)-8-methyl- 1-oxo-2H-phthalazin-6-yl]-2-methyl- pyrazol-3-yl]-4-chloro-naphthalene-2- carbonitrile. LCMS [M + 1] ⁺ 455.0. ¹H NMR (400 MHz, CD₃OD) δ = 8.65 (s, 1H), 8.51 (d, J = 8.4 Hz, 1H), 8.26-8.20 (m, 2H), 8.00 (ddd, J = 1.2, 7.2, 8.4 Hz, 1H), 7.95-7.89 (m, 1H), 7.52 (d, J = 1.2 Hz, 1H), 7.27 (d, J = 0.8 Hz, 1H), 4.28-4.14 (m, 2H), 3.79 (s, 3H), 2.61 (s, 3H) |
| 16-53 | 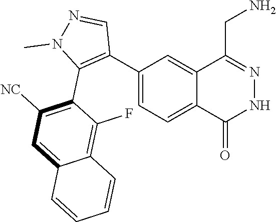 | 16-2H | 4-6 | (P)-3-[4-[4-(aminomethyl)-1-oxo-2H- phthalazin-6-yl]-2-methyl-pyrazol-3- yl]-4-fluoro-naphthalene-2- carbonitrile. LCMS [M + 1] ⁺ = 425.2. ¹H NMR (400 MHz, CD₃OD) δ = 8.48 (s, 1H), 8.30 (d, J = 8.0 Hz, 1H), 8.23 (d, J = 0.8 Hz, 1H), 8.20 (br d, J = 8.4 Hz, 1H), 8.15 (d, J = 8.4 Hz, 1H), 7.92 (dt, J = 1.2, 8.4 Hz, 2H), 7.86 (s, 1H), 7.50 (d, J = 8.4 Hz, 1H), 4.41 (br d, J = 4.4 Hz, 2H), 3.86 (s, 3H) |
| 16-54 | 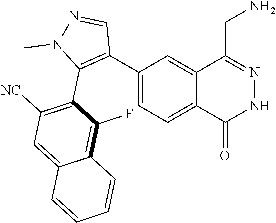 | 16-2H | 4-6 | (M)-3-[4-[4-(aminomethyl)-1-oxo-2H- phthalazin-6-yl]-2-methyl-pyrazol-3- yl]-4-fluoro-naphthalene-2- carbonitrile. LCMS [M + 1] ⁺ = 425.1. ¹H NMR (400 MHz, CD₃OD) δ = 8.48 (s, 1H), 8.30 (d, J = 8.0 Hz, 1H), 8.23 (d, J = 3.2 Hz, 1H), 8.20 (br d, J = 8.4 Hz, 1H), 8.15 (d, J = 8.4 Hz, 1H), 7.97- 7.87 (m, 2H), 7.86 (s, 1H), 7.50 (d, J = 8.4 Hz, 1H), 4.41 (br d, J = 4.0 Hz, 2H), 3.86 (s, 3H) |
| 16-55 | 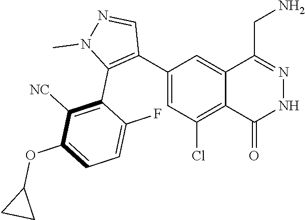 | 16-4I | 4-19 | (P)-2-[4-[4-(aminomethyl)-8-chloro- 1-oxo-2H-phthalazin-6-yl]-2-methyl- pyrazol-3-yl]-6-(cyclopropoxy)-3- fluoro-benzonitrile. LCMS [ESI, M + 1]⁺ = 465.2. ¹H NMR (400 MHz, MeOD) δ = 8.52 (s, 1H), 8.19 (s, 1H), 7.85-7.71 (m, 2H), 7.66 (d, J = 1.2 Hz, 1H), 7.43 (d, J = 1.2 Hz, 1H), 4.40-4.18 (m, 2H), 4.07 (tt, J = 2.4, 5.9 Hz, 1H), 3.82 (s, 3H), 1.07- 0.74 (m, 4H). |
| 16-56 | 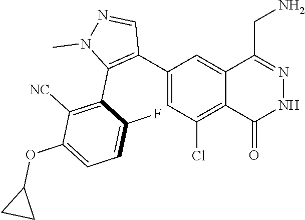 | 16-4I | 4-19 | (M)-2-[4-[4-(aminomethyl)-8-chloro- 1-oxo-2H-phthalazin-6-yl]-2-methyl- pyrazol-3-yl]-6-(cyclopropoxy)-3- fluoro-benzonitrile. LCMS [ESI, M + 1]⁺ = 465.2. ¹H NMR (400 MHz, MeOD) δ = 8.55 (s, 1H), 8.21 (s, 1H), 7.89-7.73 (m, 2H), 7.67 (d, J = 1.2 Hz, 1H), 7.46 (d, J = 1.2 Hz, 1H), 4.37-4.17 (m, 2H), 4.09 (tt, J = 2.4, 5.9 Hz, 1H), 1.02-0.77 (m, 4H). |
| 16-57 | 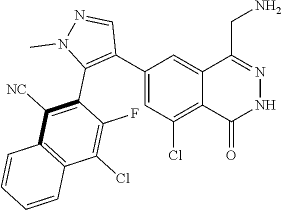 | 16-2A | 4-6 | (P)-2-[4-[4-(aminomethyl)-8-chloro- 1-oxo-2H-phthalazin-6-yl]-2-methyl- pyrazol-3-yl]-4-chloro-3-fluoro- naphthalene-1-carbonitrile. LCMS [M + 1] ⁺ 493.1. ¹H NMR (400 MHz, MeOD) δ = 8.54 (d, J = 8.4 Hz, 1H), 8.30 (d, J = 8.4 Hz, 1H), 8.27 (s, 1H), 8.04-7.98 (m, 1H), 7.96-7.91 (m, 1H), 7.70 (d, J = 1.6 Hz, 1H), 7.41 (d, J = 1.6 Hz, 1H), 4.43-4.34 (m, 1H), 4.32-4.23 (m, 1H), 3.89 (s, 3H). |
| 16-58 | 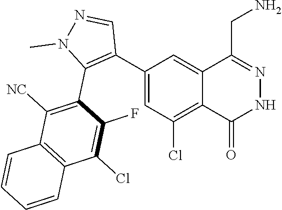 | 16-2A | 4-6 | (M)-2-[4-[4-(aminomethyl)-8-chloro- 1-oxo-2H-phthalazin-6-yl]-2-methyl- pyrazol-3-yl]-4-chloro-3-fluoro- naphthalene-1-carbonitrile. LCMS [M + 1] ⁺ 493.1. ¹H NMR (400 MHz, MeOD) δ = 8.54 (d, J = 8.8 Hz, 1H), 8.29 (d, J = 8.0 Hz, 1H), 8.27 (s, 1H), 8.05-7,98 (m, 1H), 7.97-7.90 (m, 1H), 7,70 (d, J = 1.6 Hz, 1H), 7.41 (d, J = 1.6 Hz, 1H), 4.44-4.35 (m, 1H), 4.33-4.23 (m, 1H), 3.89 (s, 3H). |
| 16-59 | 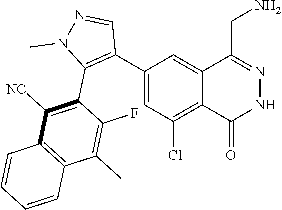 | 16-3F | 4-6 | (P)-2-[4-[4-(aminomethyl)-8-chloro- 1-oxo-2H-phthalazin-6-yl]-2-methyl- pyrazol-3-yl]-3-fluoro-4-methyl- naphthalene-1-carbonitrile. LCMS [M + 1] ⁺: 473.2. ¹H NMR (400 MHz, MeOD-d₄) δ ppm 8.36 (d, J = 8.4 Hz, 1 H) 8.25 (s, 1 H) 8.22 (d, J = 8.0 Hz, 1 H) 7.83-7.89 (m, 2 H) 7.60 (d, J = 1.2 Hz, 1 H) 7.46 (d, J = 1.2 Hz, 1 H) 3.94-4.16 (m, 2 H) 3.85 (s, 3 H) 2.81 (d, J = 2.4 Hz, 3 H) |
| 16-60 | 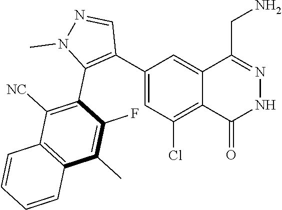 | 16-3F | 4-6 | (M)-2-[4-[4-(aminomethyl)-8-chloro- 1-oxo-2H-phthalazin-6-yl]-2-methyl- pyrazol-3-yl]-3-fluoro-4-methyl- naphthalene-1-carbonitrile. LCMS [M + 1] ⁺: 473.2. ¹H NMR (400 MHz, MeOD-d₄) δ ppm 8.36 (d, J = 8.4 Hz, 1 H) 8.26 (s, 1 H) 8.21 (d, J = 8.0 Hz, 1 H) 7.89-7.83 (m, 2 H) 7.66 (d, J = 1.2 Hz, 1 H) 7.40 (d, J = 1.2 Hz, 1 H) 4.19-4.38 (m, 2 H) 3.85 (s, 3 H) 2.81 (d, J = 2.4 Hz, 3 H) |
| 16-61 | 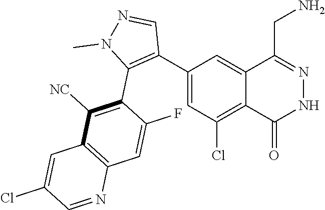 | 16-4J | 4-6 | (P)-6-[4-[4-(aminomethyl)-8-chloro- 1-oxo-2H-phthalazin-6-yl]-2-methyl- pyrazol-3-yl]-3-chloro-7-fluoro- quinoline-5-carbonitrile. LCMS [ESI, M + 1] ⁺: 494.1. ¹H NMR (400 MHz, MeOD) δ = 9.13 (d, J = 2.4 Hz, 1H), 8.58 (d, J = 1.6 Hz, 1H), 8.42 (d, J = 9.6 Hz, 1H), 8.26 (s, 1H), 7.64 (d, J = 1.6 Hz, 1H), 7.45 (d, J = 1.6 Hz, 1H), 4.40-4.32 (m, 1H), 4.30- 4.19 (m, 1H), 3.89 (s, 3H). |
| 16-62 | 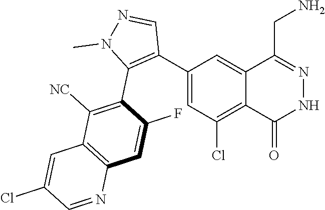 | 16-4J | 4-6 | (M)-6-[4-[4-(aminomethyl)-8-chloro- 1-oxo-2H-phthalazin-6-yl]-2-methyl- pyrazol-3-yl]-3-chloro-7-fluoro- quinoline-5-carbonitrile. LCMS [ESI, M + 1] ⁺: 494.1. ¹H NMR (400 MHz, MeOD) δ = 9.13 (d, J = 2.0 Hz, 1H), 8.58 (d, J = 2.0 Hz, 1H), 8.42 (d, J = 10.0 Hz, 1H), 8.26 (s, 1H), 7.64 (d, J = 1.2 Hz, 1H), 7.45 (d, J = 1.2 Hz, 1H), 4.40-4.33 (m, 1H), 4.30- 4.22 (m, 1H), 3.89 (s, 3H); |
| 16-63 | 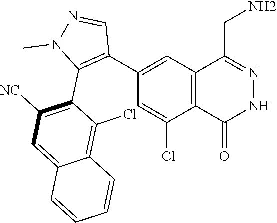 | 16-4B | 4-6 | (P)-3-[4-[4-(aminomethyl)-8-chloro- 1-oxo-2H-phthalazin-6-yl]-2-methyl- pyrazol-3-yl]-4-chloro-naphthalene-2- carbonitrile. LCMS [M + 1] ⁺: 475,2. ¹H NMR (400 MHz, MeOD) δ = 8.69 (s, 1H), 8.53- 8.48 (m, 1H), 8.29 (s, 1H), 8.25 (d, J = 8.2 Hz, 1H), 8,04-7.97 (m, 1H), 7.96- 7.89 (m, 1H), 7.69-7.64 (m, 1H), 7.40-7.35 (m, 1H), 4.28 (s, 2H), 3.79 (s, 3H) |
| 16-64 | 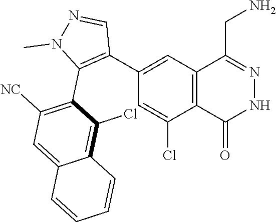 | 16-4B | 4-6 | (M)-3-[4-[4-(aminomethyl)-8-chloro- 1-oxo-2H-phthalazin-6-yl]-2-methyl- pyrazol-3-yl]-4-chloro-naphthalene-2- carbonitrile. LCMS [M + 1] ⁺: 475.2. ¹H NMR (400 MHz, MeOD) δ = 8.69 (s, 1H), 8.51 (br d, J = 8.4 Hz, 1H), 8.29 (s, 1H), 8.25 (d, J = 8.0 Hz, 1H), 8.04-7.98 (m, 1H), 7.95-7.89 (m, 1H), 7.66 (d, J = 1.2 Hz, 1H), 7.37 (d, J = 1.2 Hz, 1H), 4.28 (s, 2H), 3.79 (s, 3H) |
| 16-65 | 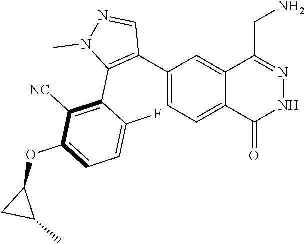 | 16-2*J | 4-21 | (P)-2-(4-(4-(aminomethyl)-1-oxo-1,2- dihydrophthalazin-6-yl)-1-methyl-1H- pyrazol-5-yl)-3-fluoro-6-((1R,2R)-2- methylcyclopropoxy)benzonitrile. LCMS [ESI, M + 1]⁺ = 445.3. ¹H NMR (400 MHz, MeOD) δ = 8.22 (d, J = 8.4 Hz, 1H), 8.18-8.10 (m, 1H), 7.85 (d, J = 1.2 Hz, 1H), 7.77-7.61(m, 2H), 7.47 (dd, J = 1.6, 8.3 Hz, 1H), 4.57-4.40 (m, 2H), 3.81 (s, 3H), 3.75- 3.67 (m, 1H), 1.25-1.11 (m, 4H), 1.01-0.90(m, 1H), 0.76-0.66 (m, 1H). |
| 16-66 | 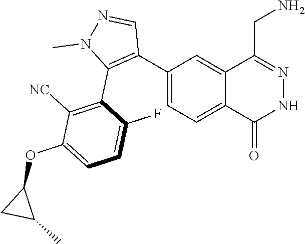 | 16-2*J | 4-21 | (M)-2-(4-(4-(aminomethyl)-1-oxo- 1,2-dihydrophthalazin-6-yl)-1-methyl- 1H-pyrazol-5-yl)-3-fluoro-6-((1R,2R)- 2-methylcyclopropoxy)benzonitrile. LCMS [ESI, M + 1]⁺ = 445.2. ¹H NMR (400 MHz, MeOD) δ = 8.22 (d, J = 8.4 Hz, 1H), 8.16 (s, 1H), 7.83 (d, J = 1.2 Hz, 1H), 7,76-7.62 (m, 2H), 7.48 (dd, J = 1.6, 8.3 Hz, 1H), 4.60-4,38 (m, 2H), 3,81 (s, 3H), 3.76-3.66 (m, 1H), 1.22-1.08 (m, 4H), 1.04-0.92 (m, 1H),0.78-0.62 (m, 1H). |
| 16-67 | 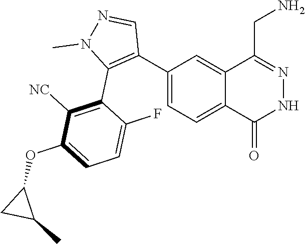 | 16-2*J | 4-21 | (P)-2-(4-(4-(aminomethyl)-1-oxo-1,2- dihydrophthalazin-6-yl)-1-methyl-1H- pyrazol-5-yl)-3-fluoro-6-((1S,2S)-2- methylcyclopropoxy)benzonitrile. LCMS [ESI, M + 1]⁺ = 445.2. ¹H NMR (400 MHz, MeOD) δ = 8.22 (d, J = 8.4 Hz, 1H), 8.15 (s, 1H), 7.87-7.82 (m, 1H), 7.77-7.59 (m, 2H), 7.51- 7.44 (m, 1H), 4.59-4.41 (m, 2H), 3.81 (s, 3H), 3.74-3.68 (m, 1H), 1.20- 1.10 (m, 4H), 1.01-0.91 (m, 1H), 0.76-0.63 (m, 1H). |
| 16-68 | 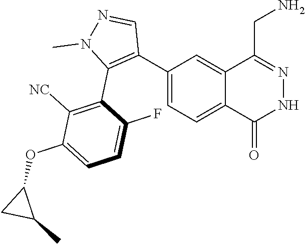 | 16-2*J | 4-21 | (M)-2-((R)-4-(4-(aminomethyl)-1- oxo-1,2-dihydrophthalazin-6-yl)-1- methyl-1H-pyrazol-5-yl)-3-fluoro-6- ((1S,2S)-2- methylcyclopropoxy)benzonitrile. LCMS [ESI, M + 1]⁺ = 445.2. ¹H NMR (400 MHz, MeOD) δ = 8.22 (d, J = 8.4 Hz, 1H), 8.16 (s, 1H), 7.84 (d, J = 1.2 Hz, 1H), 7.78-7.62 (m, 2H), 7.48 (dd, J = 1.6, 8.4 Hz, 1H), 4.59-4.42 (m, 2H), 3.81 (s, 3H), 3.76-3.68 (m, 1H), 1.22-1.13 (m, 4H), 0.99-0.90 (m, 1H),0.75-0.66 (m, 1H). |
| 16-69 | 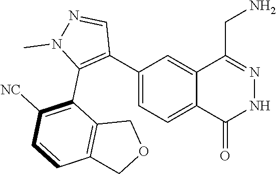 | 16- 2*C | 4-21 | (M)-4-[4-[4-(aminomethyl)-1-oxo-2H- phthalazin-6-yl]-2-methyl-pyrazol-3- yl]-1,3-dihydroisobenzofuran-5- carbonitrile. LCMS [M + 1] ⁺: 399.1. ¹H NMR (400 MHz, CD₃OD) δ = 8.23 (d, J = 8.4 Hz, 1H), 8.21 (s, 1H), 7.98 (d, J = 7.6 Hz, 1H), 7.74 (d, J = 8.0 Hz, 1H), 7.71 (d, J = 1.2 Hz, 1H), 7.54 (dd, J = 1.6, 8.4 Hz, 1H), 5.23 (s, 2H), 4.92 (br d, J = 13.6 Hz, 1H), 4.59 (br d, J = 13.6 Hz, 1H), 4.46-4.35 (m, 2H), 3.82 (s, 3H) |
| 16-70 | 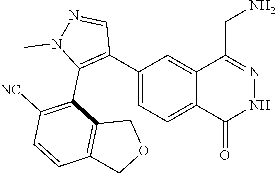 | 16- 2*C | 4-21 | (P)-4-[4-[4-(aminomethyl)-1-oxo-2H- phthalazin-6-yl]-2-methyl-pyrazol-3- yl]-1,3-dihydroisobenzofuran-5- carbonitrile. LCMS [M + 1] ⁺: 399.1. ¹H NMR (400 MHz, CD₃OD) δ = 8.24 (s, 1H), 8.21 (s, 1H), 7,98 (d, J = 7.6 Hz, 1H), 7.75 (d, J = 8.0 Hz, 1H), 7.70 (d, J = 1.2 Hz, 1H), 7.54 (dd, J = 1,2, 8.4 Hz, 1H), 5.23 (s, 2H), 4.92 (br d, J = 13.6 Hz, 1H), 4.59 (br d, J = 13.6 Hz, 1H), |
| | | | | 4.46-4.34 (m, 2H), 3.82 (s, 3H) |
| 16-71 | 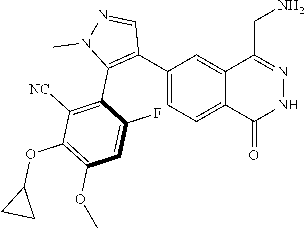 | 16-2J | 4-6 | (M)-2-[4-[4-(aminomethyl)-1-oxo-2H- phthalazin-6-yl]-2-methyl-pyrazol-3- yl]-6-(cyclopropoxy)-3-fluoro-5- methoxy-benzonitrile. LCMS [M + 1] ⁺: 461.2. ¹H NMR (400 MHz, MeOD) δ = 8.23 (d, J = 8.4 Hz, 1H), 8.16 (s, 1H), 7.85 (d, J = 1.4 Hz, 1H), 7.51 (d, J = 11.0 Hz, 1H), 7.47 (dd, J = 1.6, 8.3 Hz, 1H), 4.58-4.49 (m, 2H), 4.47-4.43 (m, 1H), 4.06 (s, 3H), 3.82 (s, 3H), 0.85-0.72 (m, 1H), 0.66-0.48 (m, 3H) |
| 16-72 | 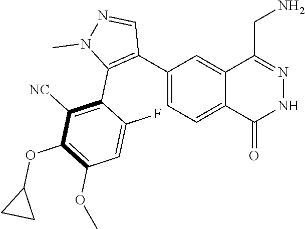 | 16-2J | 4-6 | (P)-2-[4-[4-(aminomethyl)-1-oxo-2H- phthalazin-6-yl]-2-methyl-pyrazol-3- yl]-6-(cyclopropoxy)-3-fluoro-5- methoxy-benzonitrile. LCMS [M + 1] ⁺: 461.2. ¹H NMR (400 MHz, MeOD) δ = 8.22 (d, J = 8.4 Hz, 1H), 8.16 (s, 1H), 7.84 (d, J = 1.6 Hz, 1H), 7.51 (d, J = 11.0 Hz, 1H), 7.47 (dd, J = 1.5, 8.4 Hz, 1H), 4.58-4.49 (m, 2H), 4.47-4.42 (m, 1H), 4.06 (s, 3H), 3.82 (s, 3H), 0.84-0.71 (m, 1H), 0.64-0.46 (m, 3H) |
| 16-73 | 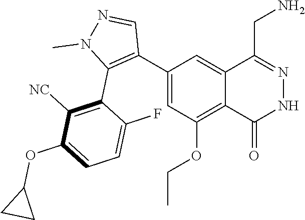 | 16-2*J | 4-6 | (P)-2-[4-[4-(aminomethyl)-8-ethoxy- 1-oxo-2H-phthalazin-6-yl]-2-methyl- pyrazol-3-yl]-6-(cyclopropoxy)-3- fluoro-benzonitrile. LCMS [ESI, M + 1] ⁺ 475.2. ¹H NMR (400 MHz, MeOD) δ = 8.20 (s, 1H), 7.87-7.68 (m, 2H), 7.31 (d, J = 1.2 Hz, 1H), 6.86 (d, J = 0.8 Hz, 1H), 4.49- 4.28 (m, 2H), 4.06-4.05 (m, 1H), 3.96-3.76 (m, 5H), 1.32-1.28 (m, 3H), 1.00-0.70 (m, 4H). |
| 16-74 | 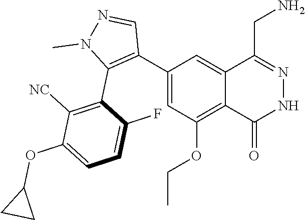 | 16-2*J | 4-6 | (M)-2-[4-[4-(aminomethyl)-8-ethoxy- 1-oxo-2H-phthalazin-6-yl]-2-methyl- pyrazol-3-yl]-6-(cyclopropoxy)-3- fluoro-benzonitrile. LCMS [ESI, M + 1] ⁺ 475.2. ¹H NMR (400 MHz, MeOD) δ = 8.19 (s, 1H), 7.86-7.71 (m, 2H), 7.33 (d, J = 1.2 Hz, 1H), 6.87 (s, 1H), 4.52-4.32 (m, 2H), 4.06-4.05 (m, 1H), 3.96-3.79 (m, 5H), 1,32-1.29 (m, 3H), 0.97- 0.75 (m, 4H). |
| 16-75 | 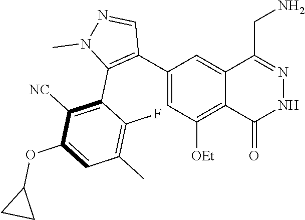 | 16- 2*C | 4-21 | (P)-2-[4-[4-(aminomethyl)-8-ethoxy- 1-oxo-2H-phthalazin-6-yl]-2-methyl- pyrazol-3-yl]-6-(cyclopropoxy)-3- fluoro-4-methyl-benzonitrile. LCMS [M + 1] ⁺ = 489.2. ¹H NMR (400 MHz, MeOD-d₄) δ = 8.18 (s, 1H), 7.68 (d, J = 6.0 Hz, 1H), 7.37 (s, 1H), 6.87 (d, J = 0.8 Hz, 1H), 4.50-4.36 (m, 2H), 4.03 (tt, J = 2.8, 5.9 Hz, 1H), 3.97-3.82 (m, 2H), 3.80 (s, 3H), 2.49 (d, J = 2.0 Hz, 3H), 1.30 (t, J = 7.2 Hz, 3H), 0.96-0.88 (m, 2H), 0.86- 0.73 (m, 2H) |
| 16-76 | 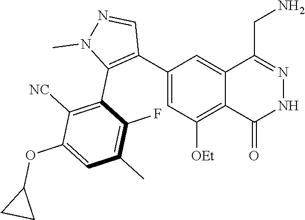 | 16- 2*C | 4-21 | (M)-2-[4-[4-(aminomethyl)-8-ethoxy- 1-oxo-2H-phthalazin-6-yl]-2-methyl- pyrazol-3-yl]-6-(cyclopropoxy)-3- fluoro-4-methyl-benzonitrile. LCMS [M + 1] ⁺ = 489.2. ¹H NMR (400 MHz, MeOD-d₄) δ = 8.20 (s, 1H), 7.70 (d, J = 6.0 Hz, 1H), 7.39 (s, 1H), 6.89 (d, J = 0.8 Hz, 1H), 4.52- 4.38 (m, 2H), 4.05 (tt, J = 2.8, 6.0 Hz, 1H), 3.98-3.83 (m, 2H), 3.82 (s, 3H), 2.51 (d, J = 2.0 Hz, 3H), 1.32 (t, J = 7.2 Hz, 3H), 0.97-0.91 (m, 2H), 0.87- 0.75 (m, 2H) |
| 16-77 | 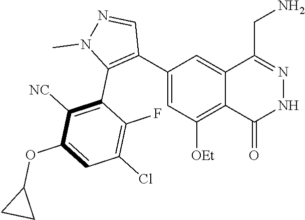 | 16- 2*C | 4-21 | (P)-2-[4-[4-(aminomethyl)-8-ethoxy- 1-oxo-2H-phthalazin-6-yl]-2-methyl- pyrazol-3-yl]-4-chloro-6- (cyclopropoxy)-3-fluoro-benzonitrile. LC-MS [M + 1]⁺ = 509.2. ¹H NMR (400 MHz, MeOD) δ = 8.20-8.16 (m, 1H), 7.98-7.92 (m, 1H), 7,39-7.31 (m, 1H), 6.92-6.84 (m, 1H), 4.50- 4.36 (m, 2H), 4.09 (tt, J = 2.8, 6.0 Hz, 1H), 4.01-3.87 (m, 2H), 3.85-3.81 (m, 3H), 1.33 (t, J = 7.2 Hz, 3H), 0.97- 0.90 (m, 2H), 0.89-0.83 (m, 1H), 0.82-0.76 (m, 1H) |
| 16-78 | 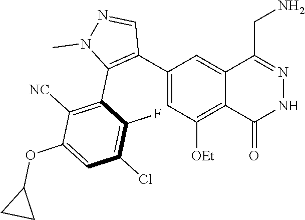 | 16- 2*C | 4-21 | (M)-2-[4-[4-(aminomethyl)-8-ethoxy- 1-oxo-2H-phthalazin-6-yl]-2-methyl- pyrazol-3-yl]-4-chloro-6- (cyclopropoxy)-3-fluoro-benzonitrile. LC-MS [M + 1]⁺ = 509.2. ¹H NMR (400 MHz, MeOD) δ = 8.22-8.17 (m, 1H), 7.99-7.94 (m, 1H), 7.40-7.35 (m, 1H), 6.90 (s, 1H), 4.57-4.34 (m, 2H), 4.17-4.04 (m, 1H), 4.02-3.89 (m, 2H), 3.85 (s, 3H), 1.35 (t, J = 7.2 Hz, 3H), 1.01-0.92 (m, 2H), 0.91- 0.85 (m, 1H), 0.84-0.77 (m, 1H) |
| 16-79 | 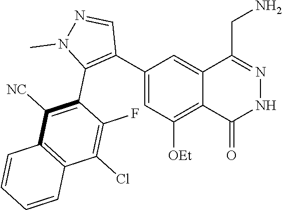 | 16-2C | 4-6 | (P)-2-[4-[4-(aminomethyl)-8-ethoxy- 1-oxo-2H-phthalazin-6-yl]-2-methyl- pyrazol-3-yl]-4-chloro-3-fluoro- naphthalene-1-carbonitrile. LCMS [M + 1] ⁺: 503.2. ¹H NMR (400 MHz, METHANOL-d₄) δ = 8.52 (d, J = 8.4 Hz, 1H), 8.30 (d, J = 8.4 Hz, 1H), 8.27 (s, 1H), 8.05-7,98 (m, 1H), 7.97-7.89 (m, 1H), 7.36 (d, J = 1.3 Hz, 1H), 6.78 (s, 1H), 4.47-4.26 (m, 2H), 3.90 (s, 3H), 3.68 (qd, J = 7.0, 9.9 Hz, 1H), 3.60-3,49 (m, 1H), 1.00 (t, J = 7,0 Hz, 3H) |
| 16-80 | 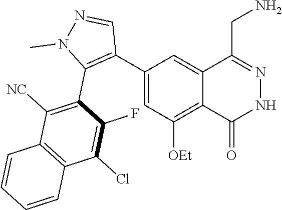 | 16-2C | 4-6 | (M)-2-[4-[4-(aminomethyl)-8-ethoxy- 1-oxo-2H-phthalazin-6-yl]-2-methyl- pyrazol-3-yl]-4-chloro-3-fluoro- naphthalene-1-carbonitrile. LCMS [M + 1] ⁺: 503.2. ¹H NMR (400 MHz, METHANOL-d4) δ = 8.52 (d, J = 8.4 Hz, 1H), 8.30 (d, J = 8.4 Hz, 1H), 8.27 (s, 1H), 8.06-7.98 (m, 1H), 7.97-7.90 (m, 1H), 7.36 (d, J = 1.1 Hz, 1H), 6.78 (s, 1H), 4.47-4.20 (m, 2H), 3.90 (s, 3H), 3.74-3.63 (m, 1H), 3.62-3.50 (m, 1H), 1.00 (t, J = 7.0 Hz, 3H) |
| 16-81 | 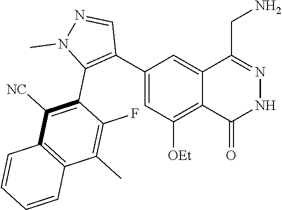 | 16-2C | 4-6 | (P)-2-[4-[4-(aminomethyl)-8-ethoxy- 1-oxo-2H-phthalazin-6-yl]-2-methyl- pyrazol-3-yl]-3-fluoro-4-methyl- naphthalene-1-carbonitrile. LC-MS [M + 1]⁺ = 483.3. ¹H NMR (400 MHz, MeOD) δ = 8.36 (d, J = 8.4 Hz, 1H), 8.27 (s, 1H), 8.23 (d, J = 8.4 Hz, 1H), 7.94-7.87 (m, 1H), 7.86-7.81 (m, 1H), 7.36 (d, J = 1.2 Hz, 1H), 6.78 (d, J = 0.8 Hz, 1H), 4.44-4.24 (m, 2H), 3.86 (s, 3H), 3.68-3.57 (m, 1H), 3.52-3.42 (m, 1H), 2.80 (d, J = 2.8 Hz, 3H), 0.96 (t, J = 7.2 Hz, 3H) |
| 16-82 | 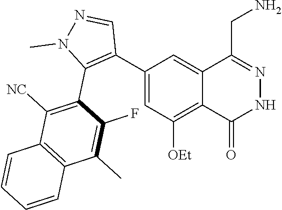 | 16-2C | 4-6 | (M)-2-[4-[4-(aminomethyl)-8-ethoxy- 1-oxo-2H-phthalazin-6-yl]-2-methyl- pyrazol-3-yl]-3-fluoro-4-methyl- naphthalene-1-carbonitrile. LC-MS [M + 1]⁺ = 483.3. ¹H NMR (400 MHz, MeOD) δ = 8.36 (d, J = 8.0 Hz, 1H), 8.26 (s, 1H), 8.25-8.21 (m, 1H), 7.94-7.88 (m, 1H), 7.87-7.82 (m, 1H), 7.37 (d, J = 1.2 Hz, 1H), 6.79 (d, J = 1.2 Hz, 1H), 4.46-4.25 (m, 2H), 3.87 (s, 3H), 3.63 (qd, J = 7.2, 10.0 Hz, 1H), 3.52-3.46 (m, 1H), 2.81 (d, J = 2.8 Hz, 3H), 0.96 (t, J = 7.2 Hz, 3H) |
| 16-83 | 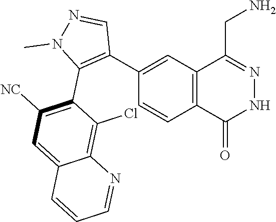 | 16-2K | 4-13 | (P)-7-(4-(4-(aminomethyl)-1-oxo-1,2- dihydrophthalazin-6-yl)-1-methyl-1H- pyrazol-5-yl)-8-chloroquinoline-6- carbonitrile. LCMS [M + 1] ⁺: 442.3. ¹H NMR (400 MHz, CD₃OD) δ = 9.27 (dd, J = 4.4, 1.6 Hz, 1H), 8.73-8.71 (m, 2H), 8.27 (s, 1H), 8.12 (d, J = 8.4 Hz, 1H), 7.96- 7.94 (m, 1H), 7.88-7.87 (m, 1H), 7.45 (dd, J = 8.4, 1.2 Hz, 1H), 4.47-4.38 (m, 2H), 3.81 (s, 3H). |
| 16-84 | 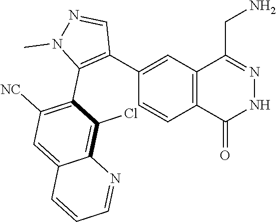 | 16-2K | 4-13 | (M)-7-(4-(4-(aminomethyl)-1-oxo-1,2- dihydrophthalazin-6-yl)-1-methyl-1H- pyrazol-5-yl)-8-chloroquinoline-6- carbonitrile. LCMS [M + 1] ⁺: 442.3. ¹H NMR (400 MHz, CD₃OD) δ = 9.25-9.24 (m, 1H), 8.68-8.63 (m, 2H), 8.25 (s, 1H), 8.13 (d, J = 8.0 Hz, 1H), 7.91-7.88 (m, 2H), 7.45(dd, J = 8.0, 1.6 Hz, 1H), 4.48-4.37 (m, 2H), 3.81 (s, 3H). |
| 16-85 | 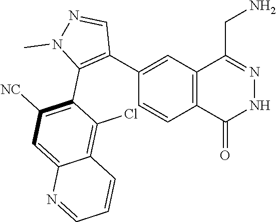 | 16-2B | 4-13 | (P)-6-(4-(4-(aminomethyl)-1-oxo-1,2- dihydrophthalazin-6-yl)-1-methyl-1H- pyrazol-5-yl)-5-chloroquinoline-7- carbonitrile. LCMS [M + 1] ⁺: 442.3. ¹H NMR (400 MHz, MeOD-d₄) δ = 9.28 (dd, J = 1.6, 4.4 Hz, 1H), 9.01 (d, J = 8.8 Hz, 1H), 8.71 (s, 1H), 8.27 (s, 1H), 8.13 (d, J = 8.4 Hz, 1H), 8.02 (dd, J = 4.4, 8.8 Hz, 1H), 7.88 (d, J = 1.2 Hz, 1H), 7.44 (dd, J = 1.6, 8.4 Hz, 1H), 4.45 (s, 2H), 3.81 (s, 3H). |
| 16-86 | 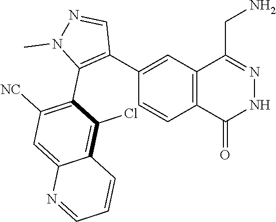 | 16-2B | 4-13 | (M)-6-(4-(4-(aminomethyl)-1-oxo-1,2- dihydrophthalazin-6-yl)-1-methyl-1H- pyrazol-5-yl)-5-chloroquinoline-7- carbonitrile LCMS [M + 1] ⁺: 442.1. ¹H NMR (400 MHz, MeOD-d₄) δ = 9.27 (dd, J = 1.6, 4.4 Hz, 1H), 9,13-8.90 (m, 1H), 8.70 (s, 1H), 8.27 (s, 1H), 8.14 (d, J = 8.4 Hz, 1H), 8.01 (dd, J = 4.4, 8.8 Hz, 1H), 7.88 (d, J = 1.6 Hz, 1H), 7.44 (dd, J = 1.6, 8.4 Hz, 1H), 4.44 (s, 2H), 3.81 (s, 3H). |
| 16-87 | 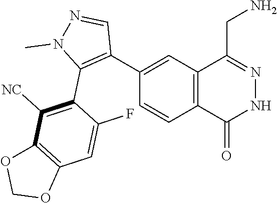 | 16-2A | 4-6 | (P)-5-[4-[4-(aminomethyl)-1-oxo-2H- phthalazin-6-yl]-2-methyl-pyrazol-3- yl]-6-fluoro-1,3-benzodioxole-4- carbonitrile. LCMS [M + 1] ⁺ = 419.1. ¹H NMR (400 MHz, MeOD-d₄) δ = 8,27 (d, J = 8.4 Hz, 1H), 8.15 (s, 1H), 7,87 (d, J = 1.2 Hz, 1H), 7.58 (dd, J = 1.6, 8.4 Hz, 1H), 7.28 (d, J = 8.8 Hz, 1H), 6.33 (s, 2H), 4.53 (d, J = 2.4 Hz, 2H), 3.82 (s, 3H) |
| 16-88 | 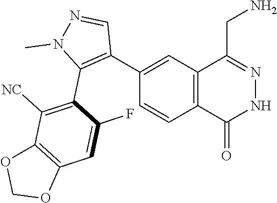 | 16-2A | 4-6 | (M)-5-[4-[4-(aminomethyl)-1-oxo- 2H-phthalazin-6-yl]-2-methyl- pyrazol-3-yl]-6-fluoro-1,3- benzodioxole-4-carbonitrile. LCMS [M + 1] ⁺ = 419.1. ¹H NMR (400 MHz, MeOD-d₄) δ = 8.27 (d, J = 8.4 Hz, 1H), 8.16 (s, 1H), 7.87 (d, J = 1.2 Hz, 1H), 7.58 (dd, J = 1.6, 8.4 Hz, 1H), 7.28 (d, J = 8.8 Hz, 1H), 6.31 (s, 2H), 4.55 (d, J = 2.4 Hz, 2H), 3.82 (s, 3H) |
| 16-89 | 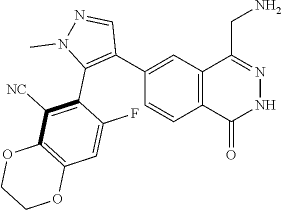 | 16- 2*C | 4-23 | (P)-6-[4-[4-(aminomethyl)-1-oxo-2H- phthalazin-6-yl]-2-methyl-pyrazol-3- yl]-7-fluoro-2,3-dihydro-1,4- benzodioxine-5-carbonitrile. LC-MS [M + 1]⁺ = 433.2. ¹H NMR (400 MHz, MeOD-d₄) δ = 8.33-8.04 (m, 2H), 7.91-7.69 (m, 1H), 7.61-7.42 (m, 1H), 7.31-7.15 (m, 1H), 4.43 (br s, 6H), 3.78 (br s, 3H) |
| 16-90 | 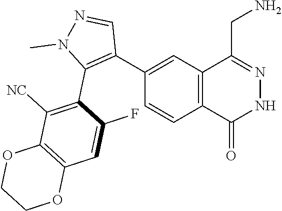 | 16- 2*C | 4-23 | (M)-6-[4-[4-(aminomethyl)-1-oxo-2H- phthalazin-6-yl]-2-methyl-pyrazol-3- yl]-7-fluoro-2,3-dihydro-1,4- benzodioxine-5-carbonitrile. LC-MS [M + 1]⁺ = 433.2. ¹H NMR (400 MHz, MeOD-d₄) δ= 8.29-8.09 (m, 2H), 7.83 (br s, 1H), 7.59-7.49 (m, 1H), 7.25 (br d, J = 8.4 Hz, 1H), 4.57- 4.41 (m, 6H), 3.79 (br s, 3H) |
| 16-91 | 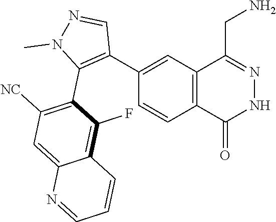 | 16-2*J | 4-6 | (P)-6-[4-[4-(aminomethyl)-1-oxo-2H- phthalazin-6-yl]-2-methyl-pyrazol-3- yl]-5-fluoro-quinoline-7-carbonitrile. LCMS [ESI, M + 1] ⁺: 426.2. ¹H NMR (400 MHz, MeOD) δ = 9.33 (d, J = 3.6 Hz, 1H), 8.95 (d, J = 8.4 Hz, 1H), 8.60 (s, 1H), 8.27 (s, 1H), 8.16 (d, J = 8.4 Hz, 1H), 8.06 (dd, J = 4.4, 8.8 Hz, 1H), 7.89 (s, 1H), 7.50 (d, J = 7.6 Hz, 1H), 4.46 (s, 2H), 3.88 (s, 3H). |
| 16-92 | 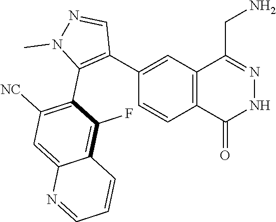 | 16-2*J | 4-6 | (M)-6-[4-[4-(aminomethyl)-1-oxo- 2H-phthalazin-6-yl]-2-methyl- pyrazol-3-yl]-5-fluoro-quinoline-7- carbonitrile. LCMS [ESI, M + 1] ⁺: 426.2. ¹H NMR (400 MHz, MeOD) δ = 9.39 (d, J = 4.0 Hz, 1H), 9.04 (br d, J = 8.4 Hz, 1H), 8.64 (s, 1H), 8.29 (s, 1H), 8.20-8.07 (m, 2H), 7.91 (s, 1H), 7.52 (d, J = 8.0 Hz, 1H), 4.49 (s, 2H), 3.91 (s, 3H). |
| 16-93 | 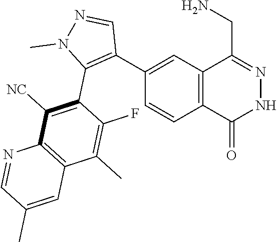 | 16-4A | 4-21 | (P)-7-(4-(4-(aminomethyl)-1-oxo-1,2- dihydrophthalazin-6-yl)-1-methyl-1H- pyrazol-5-yl)-6-fluoro-3,5- dimethylquinoline-8-carbonitrile. LCMS [ESI, M + 1] ⁺ 454.2. ¹H NMR (400 MHz, MeOD) δ = 8.91 (s, 1H), 8.52 (s, 1H), 8.21 (s, 1H), 8.10 (d, J = 8.4 Hz, 1H), 7.88 (s, 1H), 7.43 (d, 1H), 4.44 (q, J = 16.0 Hz, 2H), 3.86 (s, 3H), 2.76 (d, J = 1.6 Hz, 3H), 2,65 (s, 3H) |
| 16-94 | 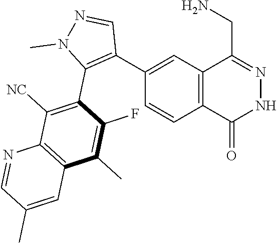 | 16-4A | 4-21 | (M)-7-(4-(4-(aminomethyl)-1-oxo-1,2- dihydrophthalazin-6-yl)-1-methyl-1H- pyrazol-5-yl)-6-fluoro-3,5- dimethylquinoline-8-carbonitrile. LCMS [ESI, M + 1] ⁺ 454.2. ¹H NMR (400 MHz, MeOD) δ = 8.92 (s, 1H), 8.53 (s, 1H), 8.22 (s, 1H), 8.11 (d, J = 8.4 Hz, 1H), 7.89 (s, 1H), 7.44 (d, J = 8.4 Hz, 1H), 4.45 (g, J = 16.4 Hz, 2H), 3.86 (s, 3H), 2.77 (d, J = 2.0 Hz, 3H), 2.65 (s, 3H). |
| 16-95 | 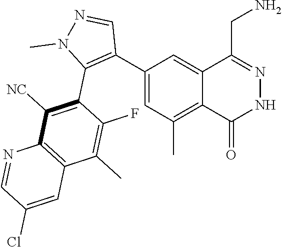 | 16-4A | 4-6 | (P)-7-(4-(4-(aminomethyl)-8-methyl- 1-oxo-1,2-dihydrophthalazin-6-yl)-1- methyl-1H-pyrazol-5-yl)-3-chloro-6- fluoro-5-methylquinoline-8- carbonitrile. LCMS [M + 1] ⁺: 488.1. ¹H NMR (400 MHz, CD₃OD) δ = 9.02 (d, J = 2.0 Hz, 1H), 8.82 (d, J = 2.4 Hz, 1H), 8.18 (s, 1H), 7.55 (s, 1H), 7.28 (s, 1H), 4.39-4.20 (m, 2H), 3.87 (s, 3H), 2.77 (d, J = 2.4 Hz, 3H), 2.64 (s, 3H). |
| 16-96 | 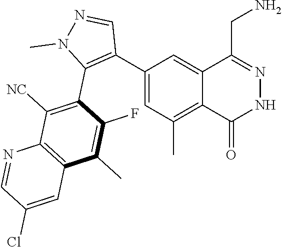 | 16-4A | 4-6 | (M)-7-(4-(4-(aminomethyl)-8-methyl- 1-oxo-1,2-dihydrophthalazin-6-yl)-1- methyl-1H-pyrazol-5-yl)-3-chloro-6- fluoro-5-methylquinoline-8- carbonitrile. LCMS [M + 1] ⁺: 488.1. ¹H NMR (400 MHz, CD3OD) 8 = 9.02 (d, J = 2.0 Hz, 1H), 8.82 (d, J = 2.4 Hz, 1H), 8.18 (s, 1H), 7.55 (d, J = 0.8 Hz, 1H), 7.28 (s, 1H), 4.43-4.19 (m, 2H), 3.87 (s, 3H), 2.77 (d, J = 2.4 Hz, 3H), 2.65 (s, 3H) |
| 16-97 | 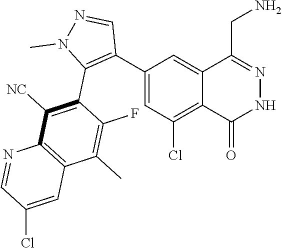 | 16-4A | 4-6 | (P)-7-(4-(4-(aminomethyl)-8-chloro- 1-oxo-1,2-dihydrophthalazin-6-yl)-1- methyl-1H-pyrazol-5-yl)-3-chloro-6- fluoro-5-methylquinoline-8- carbonitrile. LCMS [M + 1] ⁺: 508.1. ¹H NMR (400 MHz, DMSO-d₆) δ ppm 12.81 (s, 1 H) 9.18 (d, J = 2.0 Hz, 1 H) 8.99 (d, J = 2.0 Hz, 1 H) 8.51 (s, 1 H) 8.42-8.50 (m, 3 H) 7.57 (d, J = 1.6 Hz, 1 H) 7.53 (d, J = 1.2 Hz, 1 H) 4.11 (br d, J = 16.0 Hz, 1 H) 3.91 (s, 1 H) 3.83 (s, 3 H) 2.77 (d, J = 2.0 Hz, 3 H) |
| 16-98 | 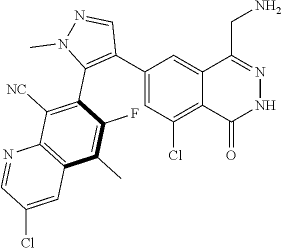 | 16-4A | 4-6 | (M)-7-(4-(4-(aminomethyl)-8-chloro- 1-oxo-1,2-dihydrophthalazin-6-yl)-1- methyl-1H-pyrazol-5-yl)-3-chloro-6- fluoro-5-methylquinoline-8- carbonitrile. LCMS [M + 1] ⁺: 508.1. ¹H NMR (400 MHz, DMSO-d₆) δ ppm 12.82 (s, 1 H) 9.18 (d, J = 2.0 Hz, 1 H) 8.99 (d, J = 2.0 Hz, 1 H) 8.52 (s, 1 H) 8.37- 8.51 (m, 3 H) 7.58 (s, 1 H) 7.53 (s, 1 H) 4.12 (br dd, J = 16.0 Hz, 1 H) 3.92 (br d, J = 4.4 Hz, 1 H) 3.83 (s, 3 H) 2.78 (d, J = 2.0 Hz, 3 H). |
| 16-99 | 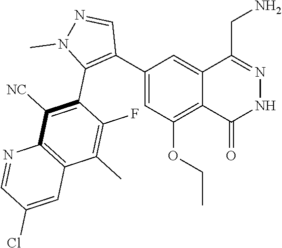 | 16-2C | 4-6 | (P)-7-(4-(4-(aminomethyl)-8-ethoxy- 1-oxo-1,2-dihydrophthalazin-6-yl)-1- methyl-1H-pyrazol-5-yl)-3-chloro-6- fluoro-5-methylquinoline-8- carbonitrile. LCMS [M +1] ⁺: 518.1. ¹H NMR (400 MHz, CD₃OD) δ = 9.03 (d, J = 2.4 Hz, 1H), 8.83 (d, J = 2.4 Hz, 1H), 8.24 (s, 1H), 7.32 (d, J = 1.2 Hz, 1H), 6.87 (d, J = 1.2 Hz, 1H), 4.44-4.22 (m, 2H), 3.87 (s, 3H), 3.82-3.72 (m, 1H), 3.69-3.59 (m, 1H), 2.77 (d, J = 2.8 Hz, 3H), 1.07 (t, J = 6.8 Hz, 3H) |
| 16-100 | 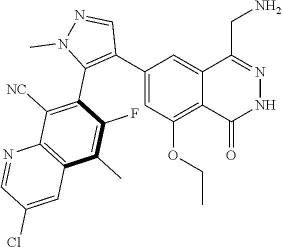 | 16-2C | 4-6 | (M)-7-(4-(4-(aminomethyl)-8-ethoxy- 1-oxo-1,2-dihydrophthalazin-6-yl)-1- methyl-1H-pyrazol-5-yl)-3-chloro-6- fluoro-5-methylquinoline-8- carbonitrile. LCMS [M + 1] ⁺: 518.1. ¹H NMR (400 MHz, CD₃OD) δ = 9.03 (d, J = 2.4 Hz, 1H), 8.83 (d, J = 2.4 Hz, 1H), 8.24 (s, 1H), 7.31 (d, J = 1.2 Hz, 1H), 6.87 (d, J = 0.8 Hz, 1H), 4.40-4.24 (m, 2H), 3.87 (s, 3H), 3.83-3.73 (m, 1H), 3.69-3.60 (m, 1H), 2.77 (d, J = 2.4 Hz, 3H), 1.07 (t, J = 7.2 Hz, 3H) |
| 16-101 | 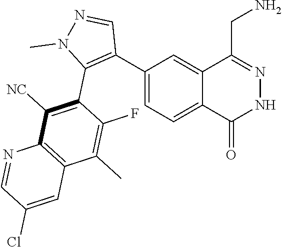 | | | (P)-6-[4-[4-(aminomethyl)-1-oxo-2H- phthalazin-6-yl]-2-methyl-pyrazol-3- yl]-3-chloro-7-fluoro-8-methyl- quinoline-5-carbonitrile. |
| 16-102 | 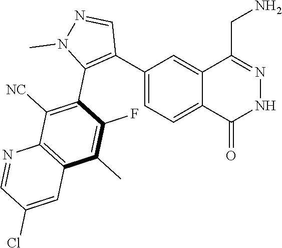 | 16- 2*C | 4-23 | (M)-6-[4-[4-(aminomethyl)-1-oxo-2H- phthalazin-6-yl]-2-methyl-pyrazol-3- yl]-3-chloro-7-fluoro-8-methyl- quinoline-5-carbonitrile. LCMS [M + 1] ⁺: 474.1. ¹H NMR (400 MHz, MeOD) δ = 9.01 (t, J = 2.0 Hz, 1H), 8.81 (s, 1H), 8.22 (s, 1H), 8.13 (dd, J = 2.8, 8.4 Hz, 1H), 7.91 (s, 1H), 7.44 (d, J = 8.4 Hz, 1H), 4.54-4.38 (m, 2H), 3.87 (s, 3H), 2.76 (d, J = 2.4 Hz, 3H) |
| 16-103 | 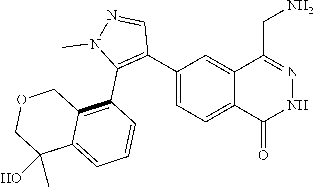 [SMILES: Cn1ncc(-c2ccc3c(=O)[nH]nc(CN)c3c2)c1-c1cccc2c1COCC2(C)O] | 16-1K | 4-18 | (M)-4-(aminomethyl)-6-(5-(4- hydroxy-4-methylisochroman-8-yl)-1- methyl-1H-pyrazol-4-yl)phthalazin- 1(2H)-one. LCMS [M + 1] ⁺: 418.2. ¹H NMR (400 MHz, CD3OD-d₄) δ ppm 8.52 (s, 1H), 8.20-8.17 (m, 2H), 7.90 (d, J = 8.0 Hz, 1H), 7.73 (dd, J = 1.6, 8.4 Hz, 1H), 7.60 (t, J = 8.0 Hz, 1H), 7.55 (d, J = 1,2 Hz, 1H), 7.36 (dd, J = 1.2, 7.6 Hz, 1H), 4.38 (d, J = 16.0 Hz, 1H), 4.24- 4.17 (m, 1H), 4.14 (d, J = 15.6 Hz, |
| | | | | 1H), 4.08-4.00 (m, 1H), 3.71-3.68 |
| | | | | (m, 3H), 3.67 (s, 1H), 3.65-3.59 (m, |
| | | | | 1H), 1.53 (s, 3H). |
| 16-104 | 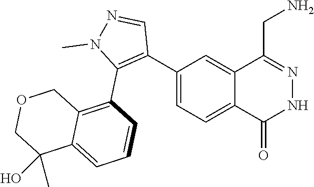 | 16-1K | 4-18 | (P)-4-(aminomethyl)-6-(5-(4- hydroxy-4-methylisochroman-8-yl)-1- methyl-1H-pyrazol-4-yl)phthalazin- 1(21)-one. LCMS [M + 1] ⁺: 418.2. ¹H NMR (400 MHz, CD3OD-d₄) δ ppm 8.50 (s, 1H), 8.23 (d, J = 8.4 Hz, 1H), 8.17 (s, 1H), 7.92-7.88 (m, 1H), 7.83 (dd, J = 1.2, 8.4 Hz, 1H), 7.60 (t. J = 7.6 Hz, 1H), 7.41 (d, J = 1.2 Hz, 1H), 7.34 (dd, J = 1.2, 7.6 1H), 4.40-4.32 (m, 1H), 4.24- 4.174 (m, 1H), 4.09 (s, 2H), 3.78- |
| | | | | 3.71 (m, 1H), 3.71-3.68 (m, 3H), |
| | | | | 3.59 (d, J = 11.2 Hz, 1H), 1.58 (s, 3H). |
| 16-105 | 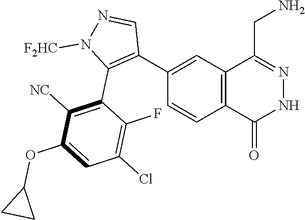 | 16-2K | 4-13 | (P)-2-(4-(4-(aminomethyl)-1-oxo-1,2- dihydrophthalazin-6-yl)-1- (difluoromethyl)-1H-pyrazol-5-yl)-4- chloro-6-cyclopropoxy-3- fluorobenzonitrile. LCMS [M + 1] ⁺: 501.1. ¹H NMR (400 MHz, MeOD-d₄) δ = 8.40 (s, 1H), 8.31 (d, J = 8.0 Hz, 1H), 8.01-7.89 (m, 2H), 7.76-7.58 (m, 1H), 7.57- 7.42 (m, 1H), 4.61-4.45 (m, 2H), 4.07 (td, J = 3.2, 6,0 Hz, 1H), 0.98- 0.88 (m, 2H), 0.88-0.79 (m, 2H). |
| 16-106 | 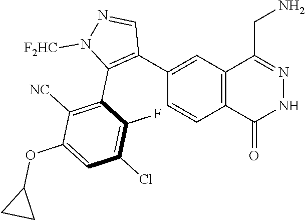 | 16-2K | 4-13 | (M)-2-(4-(4-(aminomethyl)-1-oxo-1,2- dihydrophthalazin-6-yl)-1- (difluoromethyl)-1H-pyrazol-5-yl)-4- chloro-6-cyclopropoxy-3- fluorobenzonitrile. LCMS [M + 1] ⁺: 501.0. ¹H NMR (400 MHz, MeOD-d₄) δ = 8.39 (s, 1H), 8.31 (d, J = 8.0 Hz, 1H), 7.98-7.90 (m, 2H), 7,75-7.57 (m, 1H), 7.55 (dd, J = 1.6, 8.4 Hz, 1H), 4.54 (d, J = 4.8 Hz, 2H), 4.07 (td, J = 3.2, 6.0 Hz, 1H), 1.03-0.88 (m, 2H), 0.87-0.70 (m, 2H). |
| 16-107 | 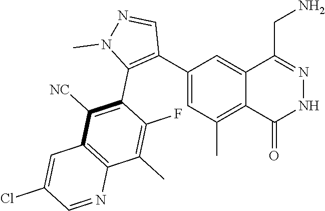 | 16-4F | 4-6 | (P)-6-(4-(4-(aminomethyl)-8-methyl- 1-oxo-1,2-dihydrophthalazin-6-yl)-1- methyl-1H-pyrazol-5-yl)-3-chloro-7- fluoro-8-methylquinoline-5- carbonitrile LC-MS [M + 1]⁺ = 488.1; ¹H NMR (400 MHz, DMSO-d₆) δ = 12.62 (s, 1H), 9.25 (d, J = 2.4 Hz, 1H), 8.55 (d, J = 2.4 Hz, 1H), 8.42 (s, 4H), 7.48 (s, 1H), 7.32 (s, 1H), 4.16-3.99 (m, 2H), 3.80 (s, 3H), 2.80 (d, J = 2.8 Hz, 3H), 2.61 (s, 3H). |
| 16-108 | 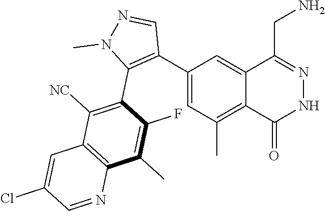 | 16-4F | 4-6 | (M)-6-(4-(4-(aminomethyl)-8-methyl- 1-oxo-1,2-dihydrophthalazin-6-yl)-1- methyl-1H-pyrazol-5-yl)-3-chloro-7- fluoro-8-methylquinoline-5- carbonitrile LC-MS [M + 1]⁺ = 488.1; ¹H NMR (400 MHz, DMSO-d₆) δ = 12.64 (s, 1H), 9.26 (d, J = 2.4 Hz, 1H), 8.56 (d, J = 2.4 Hz, 1H), 8.42 (s, 1H), 8.39 (br s, 3H), 7.48 (s, 1H), 7.34 (s, 1H), 4.18- 3.99 (m, 2H), 3.81 (s, 3H), 2.81 (d, J = 2.4 Hz, 3H), 2.62 (s, 3H). |
| 16-109 | 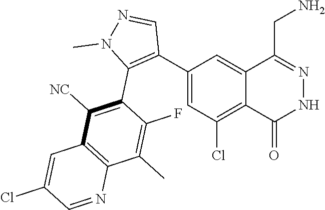 | 16-2C | 4-6 | (P)-6-(4-(4-(aminomethyl)-8-chloro- 1-oxo-1,2-dihydrophthalazin-6-yl)-1- methyl-1H-pyrazol-5-yl)-3-chloro-7- fluoro-8-methylquinoline-5- carbonitrile LCMS [M + 1] ⁺ = 508.2; ¹H NMR (400 MHz, DMSO-d₆) δ (ppm) = 12.83 (s, 1 H) 9.23-9.29 (m, 1 H) 8.56 (d, J = 2.4 Hz, 1 H) 8.51 (brd, J = 2.4 Hz, 1 H) 8.33-8.46 (m, 3 H) 7,58 (br d, J = 4.0 Hz, 1H) 7.53 (brs, 1 H) 4.12 (brd, J = 5.6 Hz, 1 H) 3.99-4.05 (m, 1 H) 3.82 (s, 3 H) 2.81 (d, J = 2.4 Hz, 3 H) |
| 16-110 | 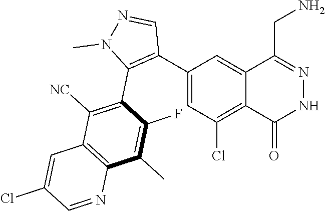 | 16-2C | 4-6 | (M)-6-(4-(4-(aminomethyl)-8-chloro- 1-oxo-1,2-dihydrophthalazin-6-yl)-1- methyl-1H-pyrazol-5-yl)-3-chloro-7- fluoro-8-methylquinoline-5- carbonitrile ¹H NMR (400 MHz, DMSO-d₆) δ (ppm) = 12.83 (s, 1 H) 9.27 (d, J = 2.0 Hz, 1 H) 8.57 (d, J = 2.4 Hz, 1 H) 8.49 (d, J = 2.0 Hz, 1 H) 8.20-8.41 (m, 3 H) 7.52-7.62 (m, 2 H) 4.13 (br d, J = 5.6 Hz, 1 H) 3.99-4.04 (m, 1 H) 3.82 (s, 3 H) 2.81 (d, J = 2.4 Hz, 3 H) |
| 16-111 | 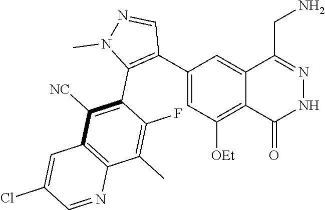 | 16-2C | 4-6 | (P)-6-(4-(4-(aminomethyl)-8-ethoxy- 1-oxo-1,2-dihydrophthalazin-6-yl)-1- methyl-1H-pyrazol-5-yl)-3-chloro-7- fluoro-8-methylquinoline-5- carbonitrile LCMS [M + 1] ⁺ = 518.3 1H NMR (400 MHz, DMSO-d₆) δ = 12.46 (s, 1H), 9.27 (d, J = 2.4 Hz, 1H), 8.57 (d, J = 2.4 Hz, 1H), 8.48 (s, 1H), 8.36 (br s, 3H), 7.27 (d, J = 1.6 Hz, 1H), 6.81 (s, 1H), 4.23-4.05 (m, 2H), 3.82 (s, 3H), 3,73-3.55 (m, 2H), 2.80 (d, J = 2.4 Hz, 3H), 0.96 (t, J = |
| | | | | 7.2 Hz, 3H) |
| 16-112 | 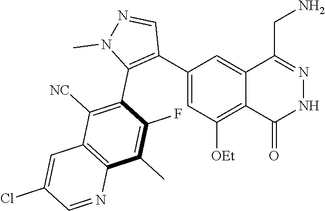 | 16-2C | 4-6 | (M)-6-(4-(4-(aminomethyl)-8-ethoxy- 1-oxo-1,2-dihydrophthalazin-6-yl)-1- methyl-1H-pyrazol-5-yl)-3-chloro-7- fluoro-8-methylquinoline-5- carbonitrile LCMS [M + 1] ⁺ = 518.3 1H NMR (400 MHz, DMSO-d₆) δ = 12.46 (s, 1H), 9.27 (d, J = 2.4 Hz, 1H), 8.57 (d, J = 2.4 Hz, 1H), 8.48 (s, 1H), 8.35 (br s, 3H), 7.27 (d, J = 1.6 Hz, 1H), 6.81 (d, J = 1.2 Hz, 1H), 4.22- 4.09 (m, 2H), 3.82 (s, 3H), 3.74- 3.56 (m, 2H), 2.80 (d, J = 2.8 Hz, 3H), |
| | | | | 0.96 (t, J = 7.2 Hz, 3H) |
| 16-113 | 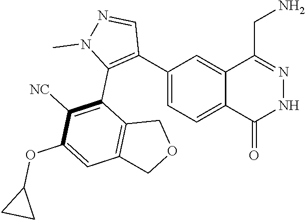 | | | (M)-4-(4-(4-(aminomethyl)-1-oxo-1,2- dihydrophthalazin-6-yl)-1-methyl-1H- pyrazol-5-yl)-6-cyclopropoxy-1,3- dihydroisobenzofuran-5-carbonitrile |
| 16-114 | 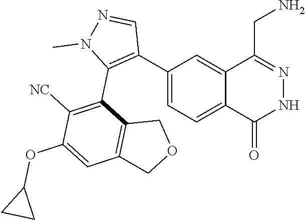 | | | (P)-4-(4-(4-(aminomethyl)-1-oxo-1,2- dihydrophthalazin-6-yl)-1-methyl-1H- pyrazol-5-yl)-6-cyclopropoxy-1,3- dihydroisobenzofuran-5-carbonitrile |
| 16-115 | 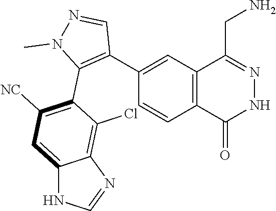 | | | (M)-5-(4-(4-(aminomethyl)-1-oxo-1,2- dihydrophthalazin-6-yl)-1-methyl-1H- pyrazol-5-yl)-4-chloro-1H- benzo[d]imidazole-6-carbonitrile |
| 16-116 | 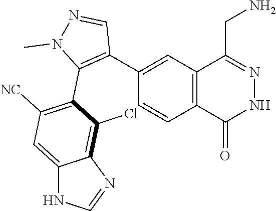 | | | (P)-5-(4-(4-(aminomethyl)-1-oxo-1,2- dihydrophthalazin-6-yl)-1-methyl-1H- pyrazol-5-yl)-4-chloro-1H- benzo[d]imidazole-6-carbonitrile |
| 16-117 | 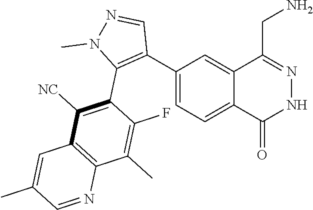 | | | (M)-6-(4-(4-(aminomethyl)-1-oxo-1,2- dihydrophthalazin-6-yl)-1-methyl-1H- pyrazol-5-yl)-7-fluoro-3,8- dimethylquinoline-5-carbonitrile |
| 16-118 | 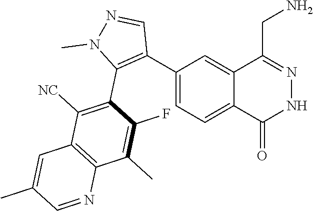 | | | (P)-6-(4-(4-(aminomethyl)-1-oxo-1,2- dihydrophthalazin-6-yl)-1-methyl-1H- pyrazol-5-yl)-7-fluoro-3,8- dimethylquinoline-5-carbonitrile |
| 16-119 | 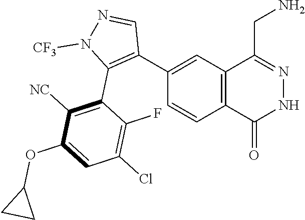 | 16-2K | 4-13 | (P)-2-(4-(4-(aminomethyl)-1-oxo-1,2- dihydrophthalazin-6-yl)-1- (trifluoromethyl)-1H-pyrazol-5-yl)-4- chloro-6-cyclopropoxy-3- fluorobenzonitrile LCMS [M + 1] ⁺: 519.1; ¹H NMR (400 MHz, MeOD-d₄) δ = 8.53 (s, 1H), 8.39-8.19 (m, 1H), 8.02-7.87 (m, 2H), 7.52 (d, J = 8.0 Hz, 1H), 4.56 (d, J = 3.2 Hz, 2H), 4.22-3.93 (m, 1H), 0.96-0.87 (m, 2H), 0.87-0.76 (m, 2H) |
| 16-120 | 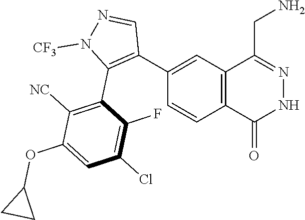 | 16-2K | 4-13 | (M)-2-(4-(4-(aminomethyl)-1-oxo-1,2- dihydrophthalazin-6-yl)-1- (trifluoromethyl)-1H-pyrazol-5-yl)-4- chloro-6-cyclopropoxy-3- fluorobenzonitrile LCMS [M + 1] ⁺: 519.1; ¹H NMR (400 MHz, MeOD-d₄) δ = 8.52 (d, J = 4.0 Hz, 1H), 8.38-8.24 (m, 1H), 8.01- 7.90 (m, 2H), 7.53 (dd, J = 1.6, 8.0 Hz, 1H), 4.55 (d, J = 3.6 Hz, 2H), 4.24- 3.94 (m, 1H), 0.91 (s, 2H), 0.83 (d, J = 2.8 Hz, 2H). |
| 16-121 | 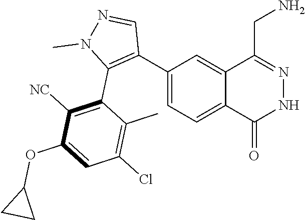 | 16-2M | 4-6 | (M)-2-(4-(4-(aminomethyl)-1-oxo-1,2- dihydrophthalazin-6-yl)-1-methyl-1H- pyrazol-5-yl)-4-chloro-6- cyclopropoxy-3-methylbenzonitrile LCMS [M + 1]⁺ 461.3; ¹H NMR (400 MHz, DMSO-d₆) δ = 12.85 (s, 1 H) 8.46-8.65 (m, 3 H) 8.42 (d, J = 1.38 Hz, 1 H) 8.12 (d, J = 8.38 Hz, 1 H) 7.86 (s, 1 H) 7.77 (d, J = 1.13 Hz, 1 H) 7.39 (dd, J = 8.32, 1.44 Hz, 1 H) 4.29- 4.37 (m, 2 H) 4.23 (b r d, J = 3.00 Hz, 1 H) 3.65 (s, 3 H) 2.01 (s, 3 H) 0.77- 0.94 (m, 4 H) |
| 16-122 | 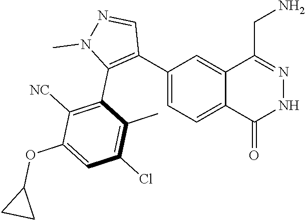 | 16-2M | 4-6 | (P)-2-(4-(4-(aminomethyl)-1-oxo-1,2- dihydrophthalazin-6-yl)-1-methyl-1H- pyrazol-5-yl)-4-chloro-6- cyclopropoxy-3-methylbenzonitrile LCMS [M + 1]⁺ 461.3; ¹H NMR (400 MHz, DMSO-d₆) δ = 12.87 (s, 1 H) 8.46-8.60 (m, 3 H) 8.38-8.45 (m, 1 H) 8.13 (d, J = 8.31 Hz, 1 H) 7.87 (s, 1 H) 7.78 (s, 1 H) 4.30-4.45 (m, 2 H) 4.23 (br d, J = 2.32 Hz, 1 H) 3.61- 3.70 (m, 3 H) 2.02 (s, 3 H) 0.79-0.95 (m, 4 H) |
| 16-123 | 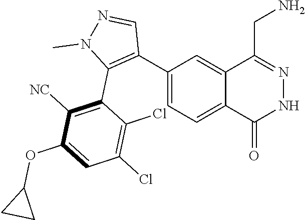 | 16-2L | 4-6 | (P)-2-(4-(4-(aminomethyl)-1-oxo-1,2- dihydrophthalazin-6-yl)-1-methyl-1H- pyrazol-5-yl)-3,4-dichloro-6- cyclopropoxybenzonitrile LCMS [M + 1]⁺ 480.9; ¹H NMR (400 MHz, MeOD) δ = 8.28-8.22 (m, 1H), 8.20-8.15 (m, 1H), 8.02-7.96 (m, 1H), 7.86 (br s, 1H), 7.44 (br d, J = 8.0 Hz, 1H), 4.52 (br s, 2H), 4.14- 4.07 (m, 1H), 3.77 (s, 3H), 0.99-0.91 (m, 2H), 0.90-0.82 (m, 2H) |
| 16-124 | 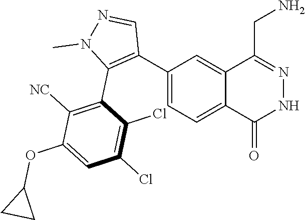 | 16-2L | 4-6 | (M)-2-(4-(4-(aminomethyl)-1-oxo-1,2- dihydrophthalazin-6-yl)-1-methyl-1H- pyrazol-5-yl)-3,4-dichloro-6- cyclopropoxybenzonitrile LCMS [M + 1]⁺ 480.9 ¹H NMR (400 MHz, MeOD-d₄) δ = 8.28-8.15 (m, 2H), 7.98 (s, 1H), 7.85 (br s, 1H), 7.44 (br d, J = 8.8 Hz, 1H), 4.59-4.48 (m, 2H), 4.20-4.06 (m, 1H), 3.76 (s, 3H), 0.94 (br dd, J = 2.8, 5.6 Hz, 2H), 0.86 (br d, J = 3.2 Hz, 2H) |
| 16-125 | 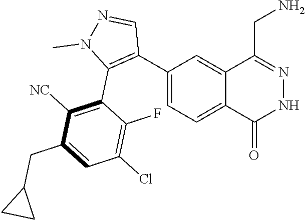 | 16-2K | 4-13 | (P)-2-(4-(4-(aminomethyl)-1-oxo-1,2- dihydrophthalazin-6-yl)-1-methyl-1H- pyrazol-5-yl)-4-chloro-6- (cyclopropylmethyl)-3- fluorobenzonitrile LCMS [M + 1]⁺ 463.2; ¹H NMR (400 MHz, MeOD-d4) δ = 8.23 (d, J = 8.4 Hz, 1H), 8.19 (s, 1H), 7.99 (d, J = 7.2 Hz, 1H), 7.85 (d, J = 1.2 Hz, 1H), 7.45 (dd, J = 1.6, 8.4 Hz, 1H), 4.58-4.44 (m, 2H), 3.84 (s, 3H), 2.75 (qd, J = 15.2, 7.2 Hz, 2H), 1.09-0.97 (m, 1H), 0.61-0.48 (m, 2H), 0.31-0.18 (m, 2H) |
| 16-126 | 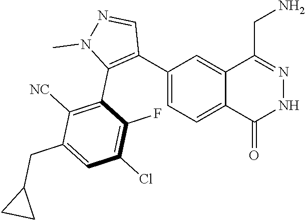 | 16-2K | 4-13 | (M)-2-(4-(4-(aminomethyl)-1-oxo-1,2- dihydrophthalazin-6-yl)-1-methyl-1H- pyrazol-5-yl)-4-chloro-6- (cyclopropylmethyl)-3- fluorobenzonitrile LCMS [M + 1]⁺ 463.3; ¹H NMR (400 MHz, DMSO-d₆) δ = 12.87 (s, 1 H) 8.46-8.60 (m, 3 H) 8.38-8.45 (m, 1 H) 8.13 (d, J = 8.31 Hz, 1 H) 7.87 (s, 1 H) 7.78 (s, 1 H) 4.30-4.45 (m, 2 H) 4.23 (br d, J = 2.32 Hz, 1 H) 3.61- 3.70 (m, 3 H) 2.02 (s, 3 H) 0.79-0.95 (m, 4 H) |
| 16-127 | 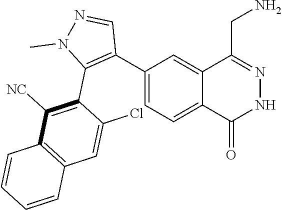 | 16-5N | 4-24 | (P)-2-(4-(4-(aminomethyl)-1-oxo-1,2- dihydrophthalazin-6-yl)-1-methyl-1H- pyrazol-5-yl)-3-chloro-1- naphthonitrile LCMS [M + 1]⁺ 441.0; ¹H NMR (400 MHz, MeOD) δ 8.65 (s, 1H), 8.26 (s, 1H), 8.21-8.11 (m, 3H), 7.90-7.81 (m, 3H), 7.45 (dd, J = 1.6, 8.4 Hz, 1H), 4.48-4.31 (m, 2H), 3.80 (s, 3H) |
| 16-128 | 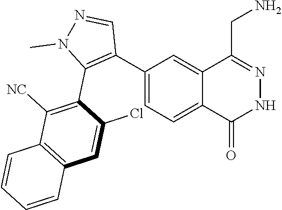 | 16-5N | 4-24 | (M)-2-(4-(4-(aminomethyl)-1-oxo-1,2- dihydrophthalazin-6-yl)-1-methyl-1H- pyrazol-5-yl)-3-chloro-1- naphthonitrile LCMS [M + 1]⁺ = 441.0; ¹H NMR (400 MHz, MeOD) δ = 8.65 (s, 1H), 8.26 (s, 1H), 8.18 (d, J = 2.4, 4.8 Hz, 2H), 8.12 (d, J = 8.4 Hz, 1H), 7.91-7.79 (m, 3H), 7.45 (d, J = 1.2, 8.4 Hz, 1H), 4.49- 4.30 (m, 2H), 3.80 (s, 3H) |
| 16-129 | 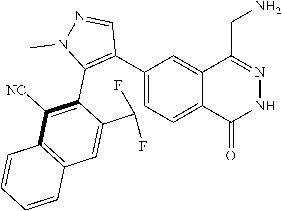 | 16-5K | 4-24 | (P)-2-(4-(4-(aminomethyl)-1-oxo-1,2- dihydrophthalazin-6-yl)-1-methyl-1H- pyrazol-5-yl)-3-(difluoromethyl)-1- naphthonitrile LCMS [M + 1]⁺ 457; ¹H NMR (400 MHz, MeOD) δ = 8.75 (s, 1H), 8.37- 8.26 (m, 3H), 8.11 (d, J = 8.4 Hz, 1H), 8.04-7.89 (m, 2H), 7.80 (s, 1H), 7.49- 7.39 (m, 1H), 6.90-6.60 (m, 1H), 4.35 (d, J = 4.4 Hz, 2H), 3.74 (s, 3H). |
| 16-130 | 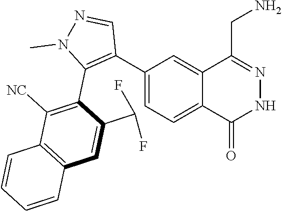 | 16-5K | 4-24 | (M)-2-(4-(4-(aminomethyl)-1-oxo-1,2- dihydrophthalazin-6-yl)-1-methyl-1H- pyrazol-5-yl)-3-(difluoromethyl)-1- naphthonitrile LCMS [M+1]⁺ 457; ¹H NMR (400 MHz, MeOD) δ = 8.65 (s, 1H), 8.26 (s, 1H), 8.18 (d, J = 2.4, 4.8 Hz, 2H), 8.12 (d, J = 8.4 Hz, 1H), 7.91-7.79 (m, 3H), 7.45 (d, J = 1.2, 8.4 Hz, 1H), 4.49-4.30 (m, 2H), 3.80 (s, 3H) |
| 16-131 | 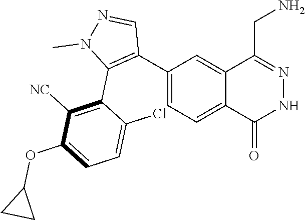 | 16-5L | 4-23 | (P)-2-(4-(4-(aminomethyl)-1-oxo-1,2- dihydrophthalazin-6-yl)-1-methyl-1H- pyrazol-5-yl)-3-chloro-6- cyclopropoxybenzonitrile LCMS [M + 1]⁺ 446.9; ¹H NMR (400 MHz, DMSO-d₆) δ = 12.85 (s, 1H), 8.50 (br s, 3H), 8.39 (s, 1H), 8.11 (dd, J = 8.8, 12.4 Hz, 2H), 7.81 (d, J = 9.2 Hz, 1H), 7.75 (s, 1H), 7.42 (d, J = 9.2 Hz, 1H), 4.29 (s, 2H), 4.23-4.15 (m, 1H), 3.69 (s, 3H), 0.98-0.74 (m, 4H) |
| 16-132 | 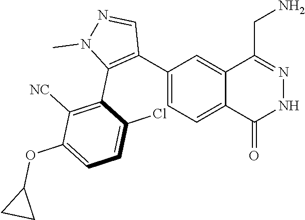 | 16-5L | 4-23 | (M)-2-(4-(4-(aminomethyl)-1-oxo-1,2- dihydrophthalazin-6-yl)-1-methyl-1H- pyrazol-5-yl)-3-chloro-6- cyclopropoxybenzonitrile LCMS [M + 1]⁺ 447.0; ¹H NMR (400 MHz, DMSO-d₆) δ = 12.87 (s, 1H), 8.51 (br s, 3H), 8.40 (s, 1H), 8.12 (dd, J = 8.8, 11.9 Hz, 2H), 7.81 (d, J = 9.2 Hz, 1H), 7.77 (d, J = 1.2 Hz, 1H), 7.43 (dd, J = 1.2, 8.4 Hz, 1H), 4.31 (br d, J = 5.6 Hz, 2H), 4.21 (td, J = 3.2, 6.0 Hz, 1H), 3.70 (s, 3H), 0.94-0.88 (m, 2H), 0.87-0.78 (m, 2H) |
| 16-133 | 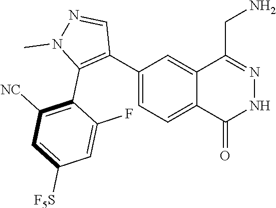 | 16-5O | 4-25 | (P)-2-(4-(4-(aminomethyl)-1-oxo-1,2- dihydrophthalazin-6-yl)-1-methyl-1H- pyrazol-5-yl)-3-fluoro-5-(pentafluoro- 16-sulfanyl)benzonitrile LCMS [M + 1]⁺ 501.2; ¹H NMR (400 MHz, DMSO-d₆) δ (ppm) = 12.91 (s, 1H), 8.78-8.67 (m, 2H), 8.57-8.43 (m, 3H), 8.39 (d, J = 1.6 Hz, 1H), 8.13 (d, J = 8.4 Hz, 1H), 7.94 (s, 1H), 7.39 (d, J = 8.4 Hz, 1H), 4.41 (br d, J = 5.2 Hz, 2H), 3.82 (s, 3H) |
| 16-134 | 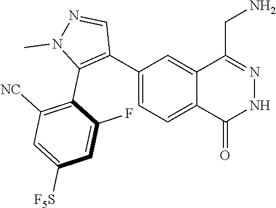 | 16-1M | 4-25 | (M)-2-(4-(4-(aminomethyl)-1-oxo-1,2- dihydrophthalazin-6-yl)-1-methyl-1H- pyrazol-5-yl)-3-fluoro-5-(pentafluoro- 16-sulfanyl)benzonitrile LCMS [M + 1]⁺ 501.2; ¹H NMR (400 MHz, DMSO-d₆) δ (ppm) = 12.90 (s, 1H), 8.76-8.66 (m, 2H), 8.54 (br s, 3H), 8.40 (s, 1H), 8.12 (d, J = 8.4 Hz, 1H), 7.95 (d, J = 1.2 Hz, 1H), 7.39 (dd, J = 1.6, 8.4 Hz, 1H), 4.41 (br d, J = 5.6 Hz, 2H), 3.81 (s, 3H) |
| 16-135 | 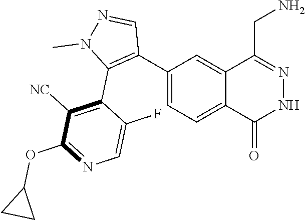 | 16-1M | 4-25 | (P)-4-(4-(4-(aminomethyl)-1-oxo-1,2- dihydrophthalazin-6-yl)-1-methyl-1H- pyrazol-5-yl)-2-cyclopropoxy-5- fluoronicotinonitrile LCMS [M + 1]⁺ 432.3; ¹H NMR (400 MHz, DMSO-d₆) δ = 12.59-12.41 (m, 1H), 8.84 (s, 1H), 8.28 (s, 1H), 8.17 (d, J = 8.4 Hz, 1H), 7.79 (d, J = 1.6 Hz, 1H), 7.72 (dd, J = 1.6, 8.4 Hz, 1H), 4.50-4.29 (m, 1H), 3.90-3.81 (m, 5H), 0.87-0.79 (m, 4H) |
| 16-136 | 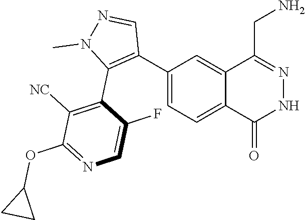 | 16-5L | 4-25 | (M)-4-(4-(4-(aminomethyl)-1-oxo-1,2- dihydrophthalazin-6-yl)-1-methyl-1H- pyrazol-5-yl)-2-cyclopropoxy-5- fluoronicotinonitrile LCMS [M + 1]⁺ 432.2; ¹H NMR (400 MHz, DMSO-d₆) δ = 12.64-12.41 (m, 1H), 8.84 (s, 1H), 8.28 (s, 1H), 8.17 (d, J = 8.3 Hz, 1H), 7.79 (s, 1H), 7.72 (dd, J = 1.6, 8.4 Hz, 1H), 4,40 (tt, J = 3.2, 6.0 Hz, 1H), 3,84 (s, 5H), 0.87- 0.79 (m, 4H) |
| 16-137 | 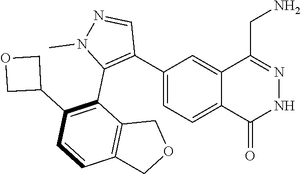 | 16-2C | 4-8 | (P)-4-(aminomethyl)-6-(1-methyl-5- (5-(oxetan-3-yl)-1,3- dihydroisobenzofuran-4-yl)-1H- pyrazol-4-yl)phthalazin-1(2H)-one LCMS [M + 1]⁺ 430.2; ¹H NMR (400 MHz, CD₃OD) δ = 8.25 (d, J = 8.4 Hz, 1H), 8.21 (s, 1H), 7.87 (d, J = 8.0 Hz, 1H), 7.81 (dd, J = 1.6, 8.4 Hz, 1H), 7.69 (d, J = 8.0 Hz, 1H), 7.39 (d, J = 1.6 Hz, 1H), 5.29-5.18 (m, 2H), 4.90 (br s, 1H), 4.81 (dd, J = 6.0, 8.4 Hz, |
| | | | | 1H), 4.78-4.72 (m, 1H), 4.66 (br |
| | | | | d, J = 13.2 Hz, 1H), 4.51-4.39 (m, |
| | | | | 2H), 4.01 (quin, J = 8.0 Hz, 1H), 3.75- |
| | | | | 3.68 (m, 2H), 3.66 (s, 3H) |
| 16-138 | 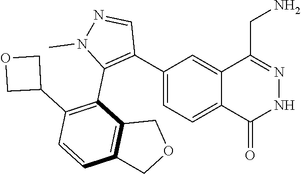 | 16-2C | 4-8 | (M)-4-(aminomethyl)-6-(1-methyl-5- (5-(oxetan-3-yl)-1,3- dihydroisobenzofuran-4-yl)-1H- pyrazol-4-yl)phthalazin-1(2H)-one LCMS [M + 1]⁺ 430.2; ¹H NMR (400 MHz, CD₃OD) δ = 8.25 (d, J = 8.4 Hz, 1H), 8.21 (s, 1H), 7.87 (d, J = 8.0 Hz, 1H), 7.80 (dd, J = 1.6, 8.4 Hz, 1H), 7.69 (d, J = 8.0 Hz, 1H), 7.39 (d, J = 1.2 Hz, 1H), 5.29-5.17 (m, 2H), 4.90 (br s, 1H), 4.81 (dd, J = 6.0, 8.4 Hz, |
| | | | | 1H), 4.78-4.73 (m, 1H), 4.66 (br |
| | | | | d, J = 13.2 Hz, 1H), 4.51-4.39 (m, |
| | | | | 2H), 4.00 (quin, J = 8.0 Hz, 1H), 3.76- |
| | | | | 3.68 (m, 2H), 3.66 (s, 3H) |

### Example 16-35 and 16-36

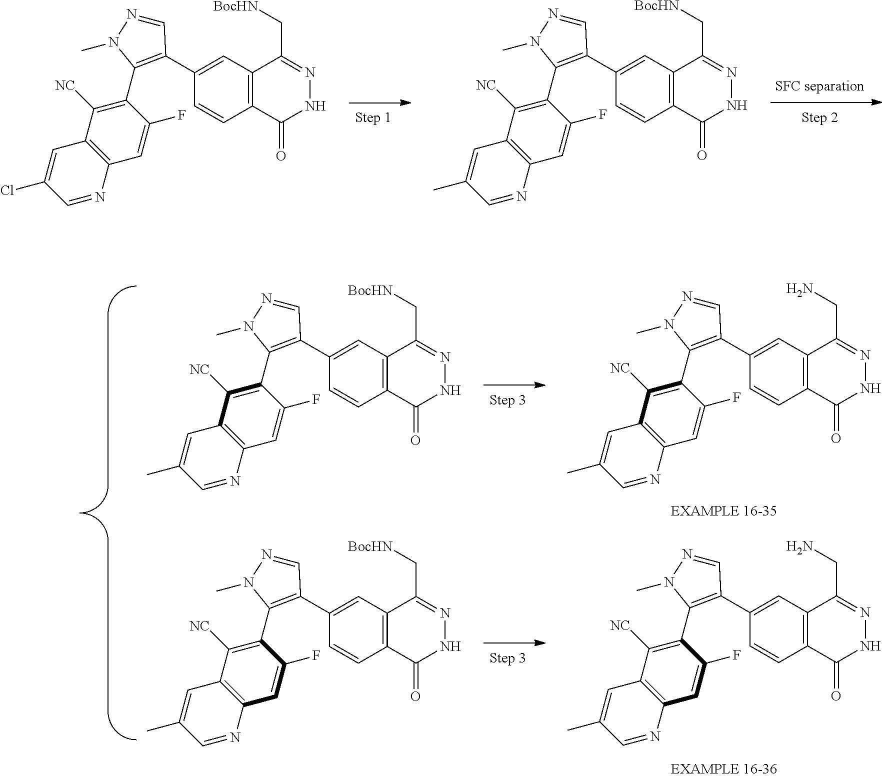

Step 1: To a solution of tert-butyl N-[[7-[5-(3-chloro-5-cyano-7-fluoro-6-quinolyl)-1-methyl-pyrazol-4-yl]-4-oxo-3H-phthalazin-1-yl]methyl]carbamate (obtained en route to Examples 16-31 and 16-32) (65.0 mg, 116 μmol, 1.00 eq.) and 2,4,6-trimethyl-1,3,5,2,4,6-trioxatriborinane (146 mg, 580 μmol, 162 μL, 50% purity, 5.00 eq.) in dioxane (1.0 mL) was added potassium carbonate (48.1 mg, 348 μmol, 3.00 eq.), followed by [1,1′-bis(diphenylphosphino)ferrocene]dichloropalladium(II) (8.49 mg, 11.6 μmol, 0.10 eq.) at 10° C. under nitrogen atmosphere. The mixture was stirred at 100° C. for 16 hours before being poured into water (10.0 mL) and extracted with ethyl acetate (10.0 mL×3). The combined organic layers were anhydrous sodium sulfate, filtered and concentrated in vacuo. The residue was purified by flash silica gel chromatography (Ethyl acetate/Petroleum ether 86%) to afford tert-butyl N-[[7-[5-(5-cyano-7-fluoro-3-methyl-6-quinolyl)-1-methyl-pyrazol-4-yl]-4-oxo-3H-phthalazin-1-yl]methyl]carbamate (40.0 mg, 62% yield) as yellow solid. LCMS [ESI, M+1]⁺: 540.3.

Step 2: Tert-butyl N-[[7-[5-(5-cyano-7-fluoro-3-methyl-6-quinolyl)-1-methyl-pyrazol-4-yl]-4-oxo-3H-phthalazin-1-yl]methyl]carbamate (43.0 mg, 79.7 μmol, 1.00 eq.) was purified by Prep-SFC (daicel chiralcel OJ (250 mm 30 mm, 10 μm); mobile phase: [0.1% ammonia hydrate metanol]; B %: 30%-30%, 3; 50 min). The collected two peaks were concentrated in vacuo to afford tert-butyl N-[[7-[5-(5-cyano-7-fluoro-3-methyl-6-quinolyl)-1-methyl-pyrazol-4-yl]-4-oxo-3H-phthalazin-1-yl]methyl]carbamate (18 mg, 41% yield peak one) as a yellow solid. (LCMS [ESI, M+1]⁺: 540.3) and Tert-butylN-[[7-[5-(5-cyano-7-fluoro-3-methyl-6-quinolyl)-1-methyl-pyrazol-4-yl]-4-oxo-3H-phthalazin-1-yl]methyl]carbamate (22 mg, 50% yield) as a yellow solid (LCMS [ESI, M+1]⁺: 540.3)

Step 3: To a solution of tert-butyl N-[[7-[5-(5-cyano-7-fluoro-3-methyl-6-quinolyl)-1-methyl-pyrazol-4-yl]-4-oxo-3H-phthalazin-1-yl]methyl]carbamate (18.0 mg, 33.4 μmol, 1.00 eq.) in methanol (1.0 mL) was added hydrogen chloride/methanol (4 M, 1.0 mL) at 10° C. The mixture was stirred at 10° C. for 1.5 hours. The mixture was concentrated in vacuo and the residue was diluted with methanol (1.0 mL) and water (10.0 mL). The resulting mixture was lyophilized to afford (P)-6-[4-[4-(aminomethyl)-1-oxo-2H-phthalazin-6-yl]-2-methyl-pyrazol-3-yl]-7-fluoro-3-methyl-quinoline-5-carbonitrile, Example 16-35 (12 mg, 74% yield) as yellow solid. LCMS [ESI, M+1]⁺: 440.2. ¹H NMR (400 MHz, MeOD-d₄) δ=9.14 (br s, 1H), 8.52 (br s, 1H), 8.36 (br d, J=8.8 Hz, 1H), 8.25 (s, 1H), 8.14 (br d, J=7.2 Hz, 1H), 7.88 (s, 1H), 7.46 (br d, J=7.2 Hz, 1H), 4.55-4.34 (m, 2H), 3.89 (s, 3H), 2.67 (s, 3H). To a solution of tert-butyl N-[[7-[5-(5-cyano-7-fluoro-3-methyl-6-quinolyl)-1-methyl-pyrazol-4-yl]-4-oxo-3H-phthalazin-1-yl]methyl]carbamate (22 mg, 40.8 μmol, 1.00 eq.) in methanol (1.0 mL) was added hydrogen chloride/methanol (4 M, 1.0 mL) at 10° C. The mixture was stirred at 10° C. for 1.5 hours. The mixture was concentrated in vacuo and the residue was diluted with methanol (1.0 mL) and water (10.0 mL). The resulting mixture was lyophilized to afford (M)-6-[4-[4-(aminomethyl)-1-oxo-2H-phthalazin-6-yl]-2-methyl-pyrazol-3-yl]-7-fluoro-3-methyl-quinoline-5-carbonitrile, Example 16-36 (18 mg, 90% yield) as a yellow solid. LCMS [ESI, M+1]⁺: 440.2. ¹H NMR (400 MHz, MeOD-d₄) δ=9.13 (s, 1H), 8.49 (s, 1H), 8.35 (br d, J=9.6 Hz, 1H), 8.25 (s, 1H), 8.14 (br d, J=8.0 Hz, 1H), 7.88 (s, 1H), 7.46 (br d, J=8.0 Hz, 1H), 4.56-4.37 (m, 2H), 3.88 (s, 3H), 2.66 (s, 3H).

### Example 16-93 and Example 16-94

Examples 16-93 and 16-94 were prepared via the same sequence as Examples 16-35 and 16-36 from tert-butyl ((7-(5-(8-cyano-6-fluoro-3,5-dimethylquinolin-7-yl)-1-methyl-1H-pyrazol-4-yl)-4-oxo-3,4-dihydrophthalazin-1-yl)methyl)carbamate (obtained en route to Examples 16-101 and 16-102) (190 mg, 331 μmol) to give (M)-7-(4-(4-(aminomethyl)-1-oxo-1,2-dihydrophthalazin-6-yl)-1-methyl-1H-pyrazol-5-yl)-6-fluoro-3,5-dimethylquinoline-8-carbonitrile (6 mg, 11.88 μmol, 65% yield) as a yellow solid; LCMS [ESI, M+1]:454.2. ¹H NMR (400 MHz, MeOD) δ=8.91 (s, 1H), 8.52 (s, 1H), 8.21 (s, 1H), 8.10 (d, J=8.4 Hz, 1H), 7.88 (s, 1H), 7.43 (d, 1H), 4.44 (q, J=16.0 Hz, 2H), 3.86 (s, 3H), 2.76 (d, J=1.6 Hz, 3H), 2.65 (s, 3H); and (P)-7-(4-(4-(aminomethyl)-1-oxo-1,2-dihydrophthalazin-6-yl)-1-methyl-1H-pyrazol-5-yl)-6-fluoro-3,5-dimethylquinoline-8-carbonitrile (7.5 mg, 8% yield) as a yellow solid. LCMS [ESI, M+1]⁺:454.2. ¹H NMR (400 MHz, MeOD) δ=8.92 (s, 1H), 8.53 (s, 1H), 8.22 (s, 1H), 8.11 (d, J=8.4 Hz, 1H), 7.89 (s, 1H), 7.44 (d, J=8.4 Hz, 1H), 4.45 (q, J=16.4 Hz, 2H), 3.86 (s, 3H), 2.77 (d, J=2.0 Hz, 3H), 2.65 (s, 3H).

### Example 16-105 and Example 16-106

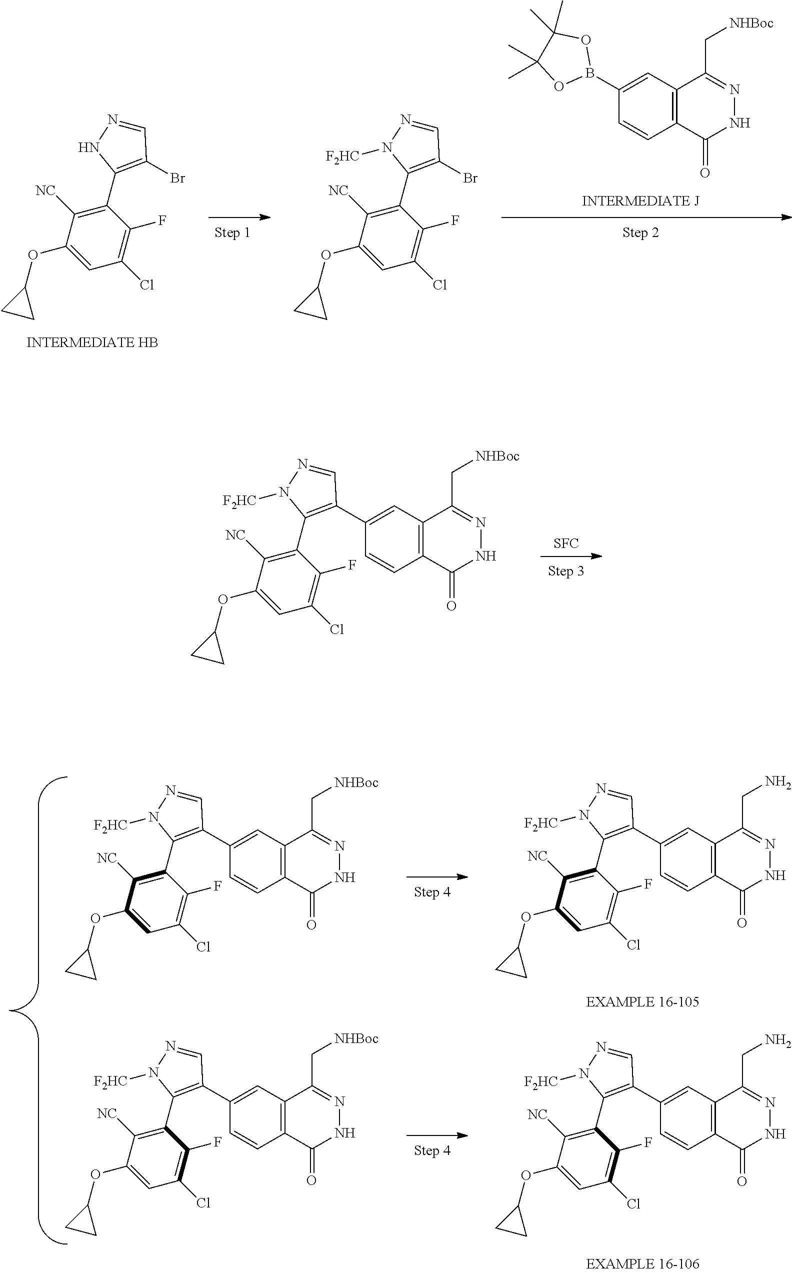

Step 1: To a solution of Intermediate HB (408 mg, 1.14 mmol, 1.00 eq.) and 1-[[bromo(difluoro)methyl]-ethoxy-phosphoryl]oxyethane (306 mg, 1.14 mmol, 1.00 eq.) in acetonitrile (8.00 mL) was added to potassium fluoride (133 mg, 2.29 mmol, 53.6 μL, 2.00 eq.) at 20° C. The mixture was stirred at 30° C. for 16 hours. The reaction mixture was concentrated under reduced pressure and the residue was purified by column chromatography (silicon dioxide, (Ethyl acetate/Petroleum ether 10-35%) to give a mixture of 2-(4-bromo-1-(difluoromethyl)-1H-pyrazol-5-yl)-4-chloro-6-cyclopropoxy-3-fluorobenzonitrile and 2-(4-bromo-1-(difluoromethyl)-1H-pyrazol-3-yl)-4-chloro-6-cyclopropoxy-3-fluorobenzonitrile (300 mg, 65% yield). The mixture was further separated by SFC (column: Phenomenex-Cellulose-2 (250 mm*30 mm, 10 μm); mobile phase: [0.1% NH₃×H₂O MeOH];B %: 5%-5%, 3; 80 min) to give 2-(4-bromo-1-(difluoromethyl)-1H-pyrazol-5-yl)-4-chloro-6-cyclopropoxy-3-fluorobenzonitrile (70.0 mg, 23.3% yield) as a white solid. ¹H NMR (400 MHz, CDCl₃) δ=7.78 (s, 1H), 7.58 (d, J=5.9 Hz, 1H), 7.17-6.98 (m, 1H), 3.94-3.83 (m, 1H), 0.98-0.92 (m, 4H).

Step 2: To a solution of 2-(4-bromo-1-(difluoromethyl)-1H-pyrazol-5-yl)-4-chloro-6-cyclopropoxy-3-fluorobenzonitrile (70.0 mg, 172 μmol, 1.00 eq.) and Intermediate J (62.2 mg, 155 μmol, 0.90 eq.) in dioxane (2.0 mL) and water (0.40 mL) was added to sodium bicarbonate (43.4 mg, 57 μmol, 3.00 eq.) and (Ad₂n-BuP)—Pd G3 (12.5 mg, 17.2 μmol, 0.10 eq.) at 15° C. The mixture was stirred at 80° C. for 16 hours under nitrogen. The mixture was diluted with water (5.0 mL) and extracted with ethyl acetate (5.0 mL×3). The organic layer was dried over sodium sulfate and then concentrated under vacuum to get the residue. The residue was purified by prep-HPLC (column: Phenomenex luna C18 150*25 mm*10 μm; mobile phase: [water (0.1% TFA)-ACN]; B %: 45%-75%, 11.5 min) to give tert-butyl ((7-(5-(3-chloro-6-cyano-5-cyclopropoxy-2-fluorophenyl)-1-(difluoromethyl)-1H-pyrazol-4-yl)-4-oxo-3,4-dihydrophthalazin-1-yl)methyl)carbamate (30.0 mg, 23.3% yield) as a white solid. LCMS [M+1]⁺: 601.2.

Step 3: The tert-butyl ((7-(5-(3-chloro-6-cyano-5-cyclopropoxy-2-fluorophenyl)-1-(difluoromethyl)-1H-pyrazol-4-yl)-4-oxo-3,4-dihydrophthalazin-1-yl)methyl)carbamate (30.0 mg, 50.0 μmol, 1.00 eq.) was separated by SFC (column: REGIS (R,R)WHELK-O1 (250 mm*25 mm, 10 um); mobile phase: [0.1% NH₃×H₂O IPA];B %: 35%-35%, 4; 35 min) to afford (peak 1) (M)-tert-butyl ((7-(5-(3-chloro-6-cyano-5-cyclopropoxy-2-fluorophenyl)-1-(difluoromethyl)-1H-pyrazol-4-yl)-4-oxo-3,4-dihydrophthalazin-1-yl)methyl)carbamate (13.0 mg, 28.3% yield) as a yellow solid and (peak 2) (P)-tert-butyl ((7-(5-(3-chloro-6-cyano-5-cyclopropoxy-2-fluorophenyl)-1-(difluoromethyl)-1H-pyrazol-4-yl)-4-oxo-3,4-dihydrophthalazin-1-yl)methyl)carbamate (13.0 mg, 40.7% yield) as a yellow solid.

Step 4: (P)-tert-butyl ((7-(5-(3-chloro-6-cyano-5-cyclopropoxy-2-fluorophenyl)-1-(difluoromethyl)-1H-pyrazol-4-yl)-4-oxo-3,4-dihydrophthalazin-1-yl)methyl)carbamate (10.0 mg, 16.6 μmol, 1.00 eq.) in DCM (0.50 mL) was added to TFA (385 mg, 3.38 mmol, 0.25 mL, 203 eq.) at 20° C. and stirred for 0.5 hour. The reaction mixture was concentrated under reduced pressure and the residue was purified by prep-HPLC (column: Welch Xtimate C18 150×25 mm×5 um; mobile phase: [water (0.05% HCl)-ACN]; B %: 20%-50%, 10 min) to give (P)-2-(4-(4-(aminomethyl)-1-oxo-1,2-dihydrophthalazin-6-yl)-1-(difluoromethyl)-1H-pyrazol-5-yl)-4-chloro-6-cyclopropoxy-3-fluorobenzonitrile (3.66 mg, 39.3% yield) as off-white solid. LCMS [M+1]⁺: 501.1. ¹H NMR (400 MHz, MeOD-d₄) δ=8.40 (s, 1H), 8.31 (d, J=8.0 Hz, 1H), 8.01-7.89 (m, 2H), 7.76-7.58 (m, 1H), 7.57-7.42 (m, 1H), 4.61-4.45 (m, 2H), 4.07 (td, J=3.2, 6.0 Hz, 1H), 0.98-0.88 (m, 2H), 0.88-0.79 (m, 2H). To a solution of (M)-tert-butyl ((7-(5-(3-chloro-6-cyano-5-cyclopropoxy-2-fluorophenyl)-1-(difluoromethyl)-1H-pyrazol-4-yl)-4-oxo-3,4-dihydrophthalazin-1-yl)methyl)carbamate (10.0 mg, 16.6 μmol, 1.00 eq.) in DCM (0.50 mL) was added to TFA (385 mg, 3.38 mmol, 0.25 mL, 203 eq.) at 20° C. and stirred for 0.5 hour. The reaction mixture was concentrated under reduced pressure and the residue was purified by prep-HPLC (column: Welch Xtimate C18 150×25 mm×5 um; mobile phase: [water (0.05% HCl)-ACN]; B %: 20%-50/o, 10 min) to give (M)-2-(4-(4-(aminomethyl)-1-oxo-1,2-dihydrophthalazin-6-yl)-1-(difluoromethyl)-1H-pyrazol-5-yl)-4-chloro-6-cyclopropoxy-3-fluorobenzonitrile (5.06 mg, 55.82% yield) as a white solid. LCMS [M+1]⁺: 501.0. ¹H NMR (400 MHz, MeOD-d₄) δ=8.39 (s, 1H), 8.31 (d, J=8.0 Hz, 1H), 7.98-7.90 (m, 2H), 7.75-7.57 (m, 1H), 7.55 (dd, J=1.6, 8.4 Hz, 1H), 4.54 (d, J=4.8 Hz, 2H), 4.07 (td, J=3.2, 6.0 Hz, 1H), 1.03-0.88 (m, 2H), 0.87-0.70 (m, 2H).

### Example 16-117 and 16-118

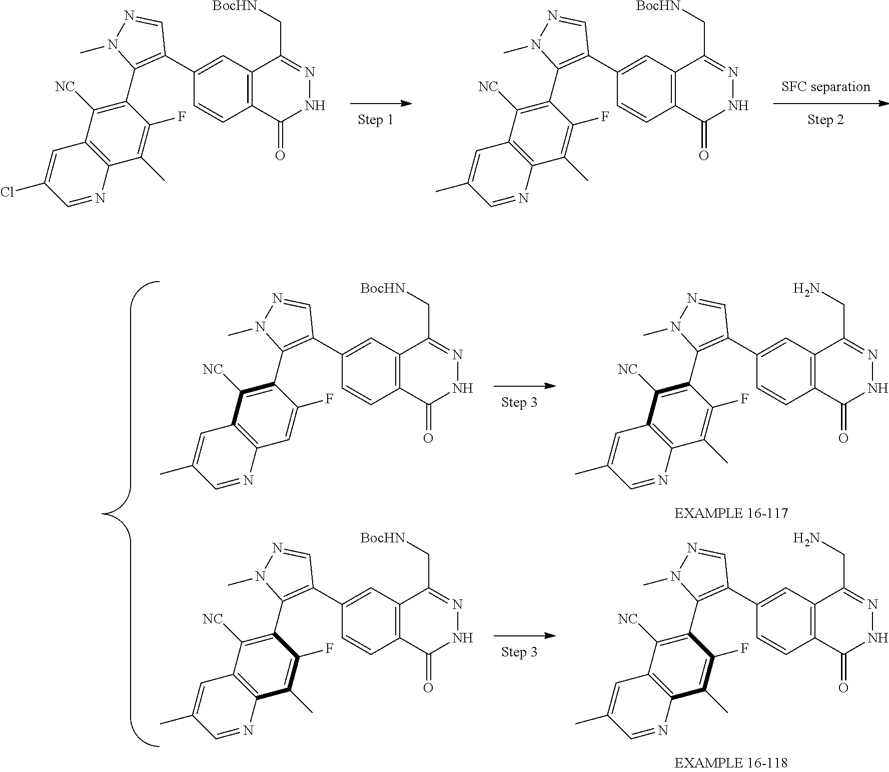

Example 16-117 (P)-6-(4-(4-(aminomethyl)-1-oxo-1,2-dihydrophthalazin-6-yl)-1-methyl-1H-pyrazol-5-yl)-7-fluoro-3,8-dimethylquinoline-5-carbonitrile and Example 16-118 and (M)-6-(4-(4-(aminomethyl)-1-oxo-1,2-dihydrophthalazin-6-yl)-1-methyl-1H-pyrazol-5-yl)-7-fluoro-3,8-dimethylquinoline-5-carbonitrile were prepared in a similar fashion to Examples 16-35 and 16-36 starting from tert-butyl N-[[7-[5-(3-chloro-5-cyano-7-fluoro-8-methyl-6-quinolyl)-1-methyl-pyrazol-4-yl]-4-oxo-3H-phthalazin-1-yl]methyl]carbamate (obtained en route to Examples 16-33 and 16-34). Example 16-117 purified by method 16-4 to give a yellow solid (34 mg, 67.6 μmol, 44% yield, hydrogen chloride. LCMS [ESI, M+1]⁺; 454.1; ¹H NMR (400 MHz, MeOD-d₄) δ=9.11 (s, 1H), 8.45 (s, 1H), 8.25 (s, 1H), 8.12 (d, J=8.4 Hz, 1H), 7.89 (s, 1H), 7.44 (br d, J=8.4 Hz, 1H), 4.46 (q, J=16.4 Hz, 2H), 3.87 (s, 3H), 2.87 (d, J=2.4 Hz, 3H), 2.65 (s, 3H); ¹⁹F NMR (400 MHz, MeOD-d₄) δ=−112.89 (br d, J=31.5 Hz, 1F) and Example 16-118 purified by method 16-4 to give a yellow solid (30.6 mg, 61.3 umol, 39.9% yield, 98.0% purity, hydrogen chloride) as yellow solid, LCMS [ESI, M+1]˜; 454.1; ¹H NMR (400 MHz, MeOD-d₄) δ=9.16 (d, J=1.6 Hz, 1H), 8.55 (s, 1H), 8.26 (s, 1H), 8.11 (d, J=8.4 Hz, 1H), 7.89 (s, 1H), 7.47-7.38 (m, 1H), 4.55-4.38 (m, 2H), 3.87 (s, 3H), 2.88 (d, J=2.4 5 Hz, 3H), 2.67 (s, 3H); ¹⁹F NMR (400 MHz, MeOD-d₄) δ=−111.64 (br s, 1F).

### Example 16-115 and 16-116

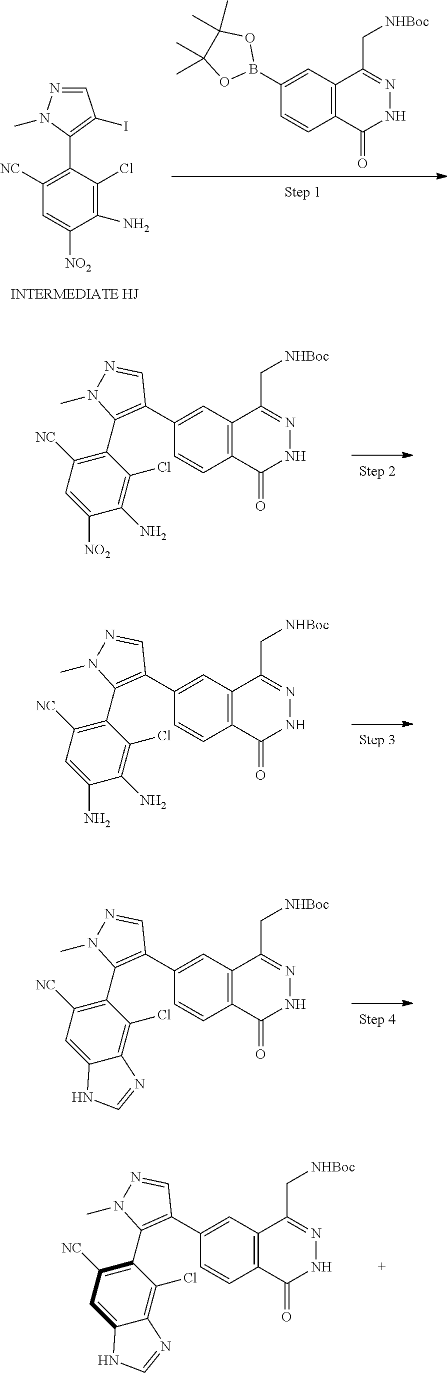

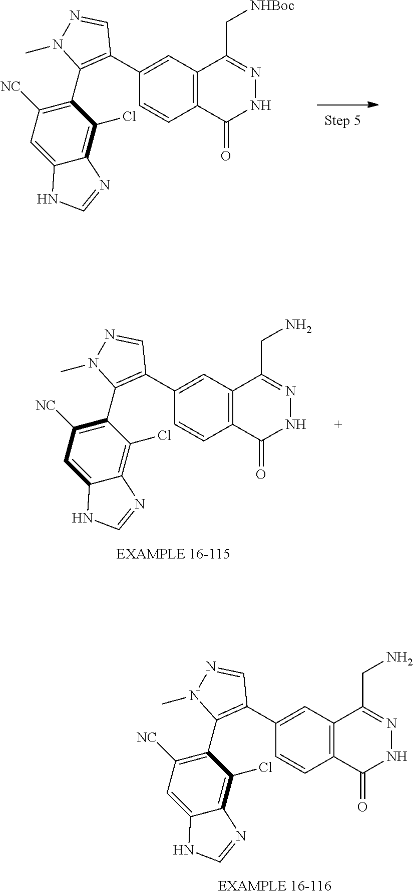

Step 1: A mixture of 2-chloro-3-(4-iodo-2-methyl-pyrazol-3-yl)-4-isocyano-6-nitro-aniline (80 mg, 198 μmol, 1.00 eq.), tert-butyl N-[[4-oxo-7-(4,4,5,5-tetramethyl-1,3,2-dioxaborolan-2-yl)-3H-phthalazin-1-yl]methyl]carbamate (95 mg, 237 μmol, 1.19 eq.), sodium bicarbonate (33 mg, 396 μmol, 2.00 eq.), [2-(2-aminophenyl)phenyl]palladium(1+); bis(1-adamantyl)-butyl-phosphane;methanesulfonate (14.4 mg, 19.8 μmol, 0.1 eq.) and water (0.5 mL) in dioxane (5.0 mL) was degassed and purged with nitrogen for 3 times, and then the mixture was stirred at 80° C. for 12 hours under nitrogen atmosphere. After being cooled to 20° C., the mixture was concentrated under vacuum. The residue was diluted with EtOAc (20 mL) and water (20 mL). The layers were separated, and the aqueous phase was extracted with EtOAc (3×20 mL). The organic layer was dried over anhydrous sodium sulfate, filtered, and concentrated under vacuum. The residue was purified by column chromatography on silica gel (petroleum ether/EtOAc=3/1 to 0/1) to give tert-butyl N-[[7-[5-(3-amino-2-chloro-6-isocyano-4-nitro-phenyl)-1-methyl-pyrazol-4-yl]-4-oxo-3H-phthalazin-1-yl]methyl]carbamate (70 mg, 116 μmol, 58% yield) as a yellow solid. ¹H NMR (400 MHz, CDCl₃-d) δ=10.01 (d, J=6.4 Hz, 1H), 8.62 (s, 1H), 8.32 (d, J=8.4 Hz, 1H), 8.03 (s, 1H), 7.61 (s, 1H), 7.57 (d, J=8.4 Hz, 1H), 5.36 (s, 1H), 4.45-4.23 (m, 2H), 3.81 (s, 3H), 1.48 (s, 9H).

Step 2: To a solution of tert-butyl N-[[7-[5-(3-amino-2-chloro-6-isocyano-4-nitro-phenyl)-1-methyl-pyrazol-4-yl]-4-oxo-3H-phthalazin-1-yl]methyl]carbamate (70 mg, 127 μmol, 1.00 eq.) in EtOAc (5.0 mL) were added ferrous powder (42.6 mg, 762 μmol, 6.00 eq.), acetic acid (11.4 mg, 191 μmol, 10.9 uL, 1.50 eq.) and water (0.3 mL). The mixture was stirred at 60° C. for 1 hour. After being cooled to 20° C., the mixture was concentrated under vacuum. The residue was diluted with EtOAc (20 mL) and water (20 mL). The layers were separated, and the aqueous phase was extracted with EtOAc (3×20 mL). The combined organic layers were dried over anhydrous sodium sulfate, filtered and concentrated under vacuum to give tert-butyl N-[[7-[5-(3,4-diamino-2-chloro-6-isocyano-phenyl)-1-methyl-pyrazol-4-yl]-4-oxo-3H-phthalazin-1-yl]methyl]carbamate (60 mg, 115 μmol, 91% yield) as a yellow solid. ¹H NMR (400 MHz, CDCl₃-d) δ=9.91 (s, 1H), 8.33 (d, J=8.4 Hz, 1H), 8.04 (s, 1H), 7.85 (d, J=10.0 Hz, 1H), 7.14-7.02 (m, 2H), 5.66 (d, J=4.0 Hz, 1H), 4.20-3.98 (m, 2H), 3.89-3.75 (m, 3H), 1.49 (s, 9H).

Step 3: To a solution of tert-butyl N-[[7-[5-(3,4-diamino-2-chloro-6-isocyano-phenyl)-1-methyl-pyrazol-4-yl]-4-oxo-3H-phthalazin-1-yl]methyl]carbamate (40.0 mg, 76.8 μmol, 1.00 eq.) in trimethoxymethane (10 mL) was added hydrochloric acid (0.5 M, 154 μL, 1.00 eq.). The mixture was stirred at 15° C. for 2 hours. After being cooled to 20° C., the mixture was concentrated under vacuum. The residue was diluted with EtOAc (20 mL) and water (20 mL). The layers were separated, and the aqueous phase was extracted with EtOAc (3×20 mL). The organic layer was dried over anhydrous sodium sulfate, filtered and concentrated under vacuum to give tert-butyl N-[[7-[5-(4-chloro-6-isocyano-1H-benzimidazol-5-yl)-1-methyl-pyrazol-4-yl]-4-oxo-3H-phthalazin-1-yl]methyl]carbamate (60 mg, crude) as a yellow solid. ¹H NMR (400 MHz, CDCl₃-d) δ=10.01 (s, 1H), 8.36 (s, 1H), 8.22 (d, J=8.0 Hz, 1H), 8.17-8.11 (m, 1H), 8.09-7.99 (m, 2H), 7.65-7.55 (m, 1H), 7.53-7.46 (m, 1H), 5.51-5.41 (m, 1H), 3.81 (s, 3H), 3.76 (s, 2H), 1.49 (s, 9H).

Step 4: tert-butyl N-[[7-[5-(4-chloro-6-isocyano-1H-benzimidazol-5-yl)-1-methyl-pyrazol-4-yl]-4-oxo-3H-phthalazin-1-yl]methyl]carbamate was separated by SFC Column: Chiralpak IC-3 50×4.6 mm, 3 μm Mobile phase: Phase A for CO₂, and Phase B for MeOH (0.05% DEA); Gradient elution: 40% MeOH (0.05% DEA) in CO₂ Flow rate: 3 mL/min; Detector: PDA; Column Temp: 35° C.; Back Pressure: 100 Bar to give compound (P)-tert-butyl N-[[7-[5-(4-chloro-6-isocyano-1H-benzimidazol-5-yl)-1-methyl-pyrazol-4-yl]-4-oxo-3H-phthalazin-1-yl]methyl]carbamate (20 mg, 38 μmol, 33% yield) as a white solid of and (M)-tert-butyl N-[[7-[5-(4-chloro-6-isocyano-1H-benzimidazol-5-yl)-1-methyl-pyrazol-4-yl]-4-oxo-3H-phthalazin-1-yl]methyl]carbamate (20 mg, 38 μmol, 33% yield) as a white solid.

Step 5: Example 16-115. To a solution of (P)-tert-butyl N-[[7-[5-(4-chloro-6-isocyano-1H-benzimidazol-5-yl)-1-methyl-pyrazol-4-yl]-4-oxo-3H-phthalazin-1-yl]methyl]carbamate (20.0 mg, 38 μmol, 1.00 eq.) in DCM (3 mL) was added TFA (924 mg, 8.10 mmol, 600 uL, 215 eq.). The mixture was stirred at 15° C. for 1 hour. The mixture was concentrated under vacuum and the residue purified by PM-4-4 to give (P)-5-(4-(4-(aminomethyl)-1-oxo-1,2-dihydrophthalazin-6-yl)-1-methyl-1H-pyrazol-5-yl)-4-chloro-1H-benzo[d]imidazole-6-carbonitrile (7.0 mg, 16 μmol, 43% yield) as a yellow solid. LCMS [M+1]⁺431.0; ¹H NMR (400 MHz, MeOD-d₄) δ=9.25-9.06 (m, 1H), 8.37 (d, J=1.6 Hz, 1H), 8.26 (s, 1H), 8.13 (d, J=8.4 Hz, 1H), 7.88 (s, 1H), 7.39 (d, J=8.4 Hz, 1H), 4.47 (s, 2H), 3.78 (s, 3H).

Example 16-116. To a solution of (M)-tert-butyl N-[[7-[5-(4-chloro-6-isocyano-1H-benzimidazol-5-yl)-1-methyl-pyrazol-4-yl]-4-oxo-3H-phthalazin-1-yl]methyl]carbamate (20.0 mg, 38 μmol, 1.00 eq.) in DCM (3 mL) was added TFA (924 mg, 8.10 mmol, 0.6 mL, 215 eq.). The mixture was stirred at 15° C. for 1 hour. The mixture was concentrated under vacuum and the residue purified by PM-4-4 to give (M)-5-(4-(4-(aminomethyl)-1-oxo-1,2-dihydrophthalazin-6-yl)-1-methyl-1H-pyrazol-5-yl)-4-chloro-1H-benzo[d]imidazole-6-carbonitrile (5.9 mg, 13.5 mol, 36% yield) as an off-white solid. LCMS [M+1]⁺431.0; ¹H NMR (400 MHz, MeOD-d₄) δ=9.14-8.96 (m, 1H), 8.42-8.30 (m, 1H), 8.25 (d, J=3.2 Hz, 1H), 8.13 (d, J=8.4 Hz, 1H), 7.89 (s, 1H), 7.38 (dd, J=1.6, 8.4 Hz, 1H), 4.47 (br d, J=3.2 Hz, 2H), 3.78 (s, 3H).

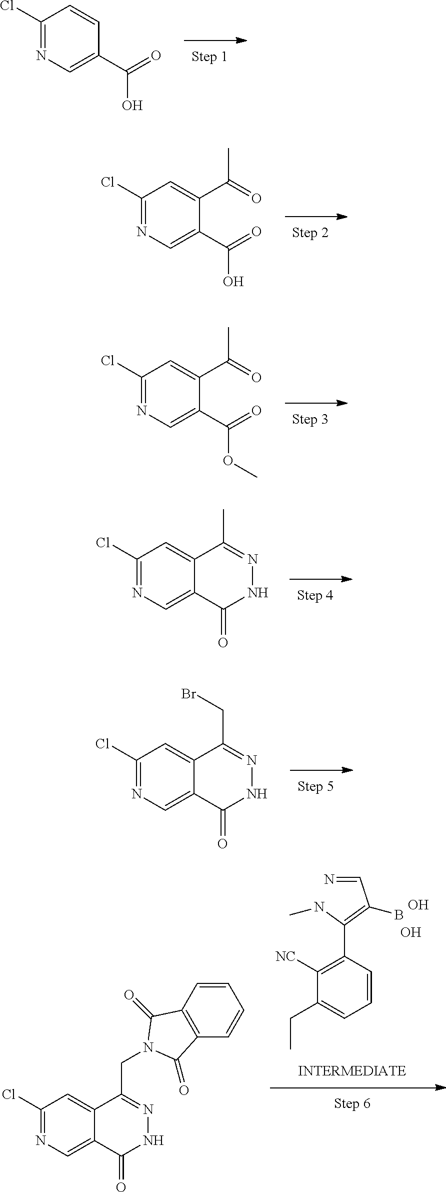

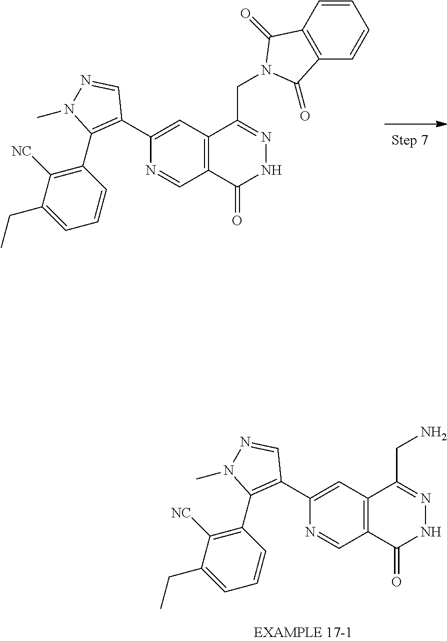 [SMILES: CCc1cccc(-c2c(-c3cc4c(CN5C(=O)c6ccccc6C5=O)n[nH]c(=O)c4cn3)cnn2C)c1C#N ; CCc1cccc(-c2c(-c3cc4c(CN)n[nH]c(=O)c4cn3)cnn2C)c1C#N]

### Example 17-1

Step 1: To a mixture of n-BuLi (2.50 M, 50.8 mL, 4.00 eq) in THF (50 mL) was added 2,2,6,6-tetramethylpiperidine (13.4 g, 95.2 mmol, 16.2 mL, 3.00 eq) at −78° C. under N₂ atmosphere. The mixture was warmed to 0° C. and cooled again to −78° C. 6-Chloropyridine-3-carboxylic acid (5.00 g, 31.7 mmol, 1.00 eq) in THF (50 mL) was added to the mixture drop-wise. Then the mixture was stirred at −78° C. for 2 hours before N-methoxy-N-methyl-acetamide (6.55 g, 63.5 mmol, 6.75 mL, 2.00 eq) was added to the mixture and the resulting mixture was stirred at −78° C. for 1 hour. The reaction mixture was quenched with water (20 mL), pH was adjusted to 4 by aqueous HCl (1.00 N). The aqueous layer was extracted with ethyl acetate (20 mL×2). The combined organic layers were dried over anhydrous sodium sulfate, filtered and concentrated in vacuo to give 4-acetyl-6-chloro-pyridine-3-carboxylic acid (4.50 g, 21.3% yield, 30.0% purity) as a yellow solid which used for the next step without further purification. LCMS [M+1]⁺=200.2

Step 2: To a mixture of 4-acetyl-6-chloro-pyridine-3-carboxylic acid (4.50 g, 22.6 mmol, 1.00 eq) in methanol (10.0 mL) and DCM (50 mL) was added TMS-Diazomethane (5.15 g, 45.1 mmol, 2.00 eq) and the resulting mixture was stirred at 20° C. for 1 hour. The reaction was quenched with methyl alcohol (25 mL) and concentrated under reduced pressure and the residue was purified by flash silica gel chromatography (Ethyl acetate/Petroleum ether 0-50%) to give methyl 4-acetyl-6-chloro-pyridine-3-carboxylate (1.05 g, 21.8% yield) as a yellow solid. LCMS [M+1]⁺=214.2

Step 3: To a solution of methyl 4-acetyl-6-chloro-pyridine-3-carboxylate (1.05 g, 4.92 mmol, 1.00 eq) in ethyl alcohol (10.0 mL) was added hydrazine hydrate (251 mg, 4.92 mmol, 244 μL, 98% purity, 1.00 eq) and the mixture was stirred at 75° C. for 1 hour. The reaction mixture was concentrated in vacuo. The crude product 7-chloro-1-methyl-3H-pyrido[3,4-d]pyridazin-4-one (950 mg, 98.8% yield), a yellow solid, was used in the next step without further purification. LCMS [M+1]⁺=196.3. ¹H NMR (400 MHz, METHANOL-d₄) δ=9.25 (s, 1H), 8.02 (s, 1H), 2.55 (s, 3H).

Step 4: To a solution of 7-chloro-1-methyl-3H-pyrido[3,4-d]pyridazin-4-one (300 mg, 1.53 mmol, 1.00 eq) in chloroform (10.0 mL) was added AIBN (25.2 mg, 153 μmol, 0.10 eq) and NBS (300 mg, 1.69 mmol, 1.10 eq) and the mixture was stirred at 80° C. for 5 hours The mixture was concentrated in vacuo. The residue was purified by flash silica gel chromatography (Ethyl acetate/Petroleum ether 0-35%) to give 1-(bromomethyl)-7-chloropyrido[3,4-d]pyridazin-4 (3H)-one (31.0 mg, 7.36% yield) as a white solid. ¹H NMR (400 MHz, METHANOL-d₄) δ=9.27 (s, 1H), 8.16 (s, 1H), 4.96 (s, 2H).

Step 5: To a solution of 1-(bromomethyl)-7-chloro-3H-pyrido[3,4-d]pyridazin-4-one (80.0 mg, 291 μmol, 1.00 eq) in DMF (3.0 mL) was added potassium carbonate (80.6 mg, 583 μmol, 2.00 eq) and isoindoline-1,3-dione (42.9 mg, 291 μmol, 1.00 eq). The mixture was stirred at 20° C. for 12 hours. LCMS showed the desired MS was found. The mixture was concentrated in vacuo. The residue was purified by prep-TLC (SiO₂, Petroleum ether/Ethyl acetate=1:1) to give 2-[(7-chloro-4-oxo-3H-pyrido[3,4-d]pyridazin-1-yl)methyl]isoindoline-1,3-dione (22 mg, 22.2% yield) as a white solid. LCMS [M+1]⁺=341.2

Step 6: To a solution of 2-[(7-chloro-4-oxo-3H-pyrido[3,4-d]pyridazin-1-yl)methyl]isoindoline-1,3-dione (10.0 mg, 29.4 μmol, 1.00 eq) in DMF (0.50 mL) and water (0.10 mL) was added [5-(2-cyano-3-ethyl-phenyl)-1-methyl-pyrazol-4-yl]boronic acid (15.0 mg, 58.7 μmol, 2.00 eq), potassium phosphate (18.7 mg, 88.1 μmol, 3.00 eq) and Xphos-Pd-G2 (2.31 mg, 2.93 μmol, 0.10 eq). The resulting mixture was degassed and stirred at 100° C. for 1 hour. The mixture was concentrated in vacuo. The residue was purified by prep-HPLC (TFA condition: column: Boston Green ODS 150×30 mm×5 μm; mobile phase: [water (0.1% TFA)-ACN];B %: 38%-58%, 10 min] to give 2-[4-[1-[(1,3-dioxoisoindolin-2-yl)methyl]-4-oxo-3H-pyrido[3,4-d]pyridazin-7-yl]-2-methyl-pyrazol-3-yl]-6-ethylbenzonitrile (3.00 mg, 19.8% yield) as a white solid.

Step 7: To a solution of 2-[4-[1-[(1,3-dioxoisoindolin-2-yl)methyl]-4-oxo-3H-pyrido[3,4-d]pyridazin-7-yl]-2-methyl-pyrazol-3-yl]-6-ethyl-benzonitrile (3.00 mg, 5.82 μmol, 1.00 eq) in dioxane (1.0 mL) was added hydrazine hydrate (2.97 mg, 58.19 μmol, 2.89 μL, 98% purity, 10.0 eq) and the mixture was stirred at 20° C. for 8 hours The mixture was concentrated in vacuo. The residue was purified by prep-HPLC (TFA condition: column: Boston Green ODS 150×30 mm×5 μm; mobile phase: [water (0.1% TFA)-ACN];B %: 50/6-80%, 10 min) to give 2-[4-[1-(aminomethyl)-4-oxo-3H-pyrido[3,4-d]pyridazin-7-yl]-2-methyl-pyrazol-3-yl]-6-ethyl-benzonitrile (1.40 mg, 3.63 μmol, 62.4% yield) as a white solid. LCMS [M+1]⁺=386.4. ¹H NMR (400 MHz, METHANOL-d₄) δ=9.20 (s, 1H), 8.37 (s, 1H), 7.81-7.76 (m, 1H), 7.74 (s, 1H), 7.67-7.62 (m, 1H), 7.48 (br d, J=7.2 Hz, 1H), 4.50-4.35 (m, 2H), 3.82-3.73 (m, 3H), 3.00-2.88 (m, 2H), 1.33 (br t, J=7.6 Hz, 3H).

### Example 17-2

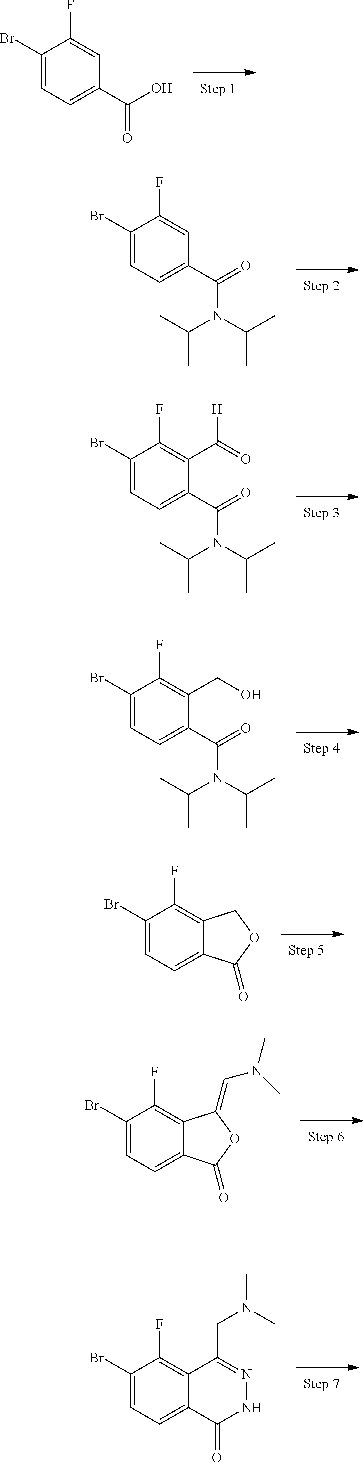

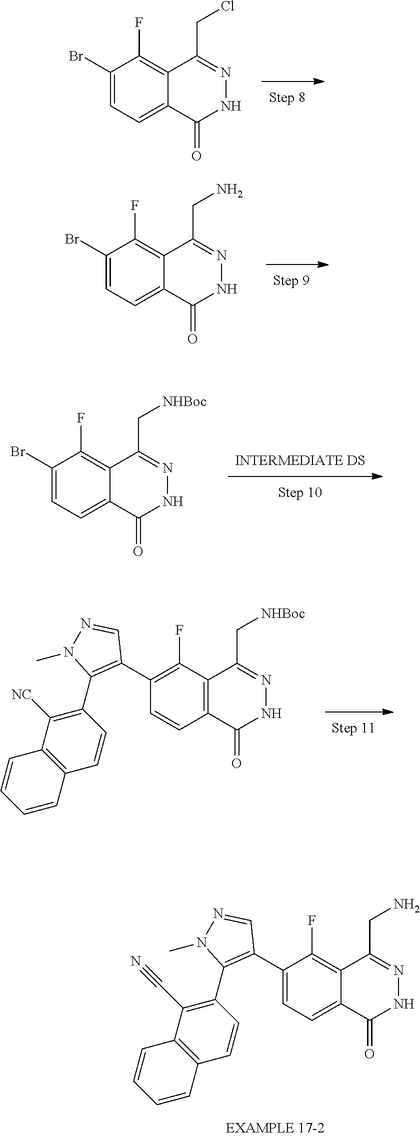

Step 1: To a solution of 4-bromo-3-fluoro-benzoic acid (16.0 g, 73.1 mmol, 1.00 eq.) in dichloromethane (200 mL) was added oxalyl chloride (13.9 g, 109 mmol, 9.59 mL, 1.50 eq.) and N.N-dimethylformamide (534 mg, 7.31 mmol, 562 μL, 0.10 eq.) at 25° C. The reaction mixture was stirred at 25° C. for 0.5 hour before diisopropylethylamine (28.3 g, 219 mmol, 3.00 eq.) was added followed by N-isopropylpropan-2-amine (14.8 g, 146 mmol, 20.6 mL, 2.00 eq.) at 0° C. The reaction mixture was stirred at 0° C. for 1.5 hours. The reaction mixture was quenched with water (150 mL) at 25° C., and layers were separated. The aqueous layer was extracted with ethyl acetate (100 mL). The combined organic layers were dried over anhydrous sodium sulfate and concentrated under reduced pressure and the residue was purified by column chromatography (silica gel, ethyl acetate/petroleum ether 10-20/6) to give 4-bromo-3-fluoro-N,N-diisopropyl-benzamide (21.1 g, 95.0% yield) as a yellow oil. ¹H NMR (400 MHz, CDCl₃) δ=7.50 (dd, 0.1=6.8, 8.0 Hz, 1H), 7.02 (dd, J=2.0, 8.4 Hz, 1H), 6.91 (dd, J=1.6, 8.0 Hz, 1H), 3.89-3.23 (m, 2H), 1.53-1.02 (m, 12H).

Step 2: To a solution of 2,2,6,6-tetramethylpiperidine (9.35 g, 66.2 mmol, 11.2 mL, 2.00 eq.) in THF (100 mL) was added n-butyllithium (2.50 M, 26.5 mL, 2.00 eq.) drop-wise at −60° C. and the resulting solution was stirred for 1 h. Following that a solution of 4-bromo-3-fluoro-N,N-diisopropyl-benzamide (10.0 g, 33.1 mmol, 1.00 eq.) in THF (20 mL) was added to the reaction mixture dropwise at −60° C. The mixture was stirred at −60° C. for 1 hour. After that a solution of N,N-dimethylformamide (19.3 g, 265 mmol, 20.4 mL, 8.00 eq.) in THF (20 mL) was added dropwise into the reaction mixture. The mixture was stirred at −60° C. for 3 hours and then warmed up to 25° C. The mixture was quenched with water (100 mL) at 0° C. and extracted with ethyl acetate (100 mL×2). The combined organic layers were dried over anhydrous sodium sulfate, filtered and concentrated under reduced pressure and the residue was purified by column chromatography (silica gel, ethyl acetate/petroleum ether 0-20%) to give 4-bromo-3-fluoro-2-formyl-N,N-diisopropyl-benzamide (9.50 g, 79.0% yield) as a yellow solid. LCMS [M+1]⁺: 330.0.

Step 3: To a solution of 4-bromo-3-fluoro-2-formyl-N,N-diisopropyl-benzamide (2.00 g, 6.06 mmol, 1.00 eq.) in methyl alcohol (20 mL) was added sodium borohydride (458 mg, 12.1 mmol, 2.00 eq.) at 0° C. The mixture was stirred at 25° C. for 3 hours. The reaction mixture was quenched with water (40.0 mL) at 0° C. and extracted with ethyl acetate (40.0 mL×2). The combined organic layers were dried over anhydrous sodium sulfate and concentrated under reduced pressure to give 4-bromo-3-fluoro-2-(hydroxymethyl)-N,N-diisopropyl-benzamide (2.00 g, crude) as a yellow solid. LCMS[M+1]⁺: 332.1.

Step 4: A solution of 4-bromo-3-fluoro-2-(hydroxymethyl)-N,N-diisopropyl-benzamide (50.0 mg, 150 μmol, 1.00 eq.) in hydrochloric acid (6.00 M, 502 μL, 20.0 eq.) was stirred at 100° C. for 15 hours. The reaction mixture was adjusted to pH=8 by sodium bicarbonate aqueous solution at 0° C. and extracted with ethyl acetate (15 mL×2). The combined organic layers were dried over anhydrous sodium sulfate and concentrated under reduced pressure to give 5-bromo-4-fluoro-3H-isobenzofuran-1-one (34.0 mg, crude) as a yellow solid. ¹H NMR (400 MHz, CDCl₃) δ=7.77 (dd, J=6.0, 8.0 Hz, 1H), 7.62 (d, J=8.0 Hz, 1H), 5.39 (s, 2H). LCMS[M+1]⁺: 231.0.

Step 5: A mixture of 5-bromo-4-fluoro-3H-isobenzofuran-1-one (1 g, 4.33 mmol, 1 eq.) and potassium tert-butoxide (48.6 mg, 433 μmol, 0.10 eq.) in N,N-dimethylformamide (3.61 g, 30.3 mmol, 4.03 mL, 7.00 eq.) was stirred at 100° C. for 3 hours under nitrogen. Water (25 mL) was added and the mixture was extracted with ethyl acetate (30 mL×3). The combined organic phase was washed with brine (30 mL×3), dried over anhydrous sodium sulfate, filtered and concentrated in vacuo. The residue was purified by silica gel column chromatography (silica gel, ethyl acetate/petroleum ether 1-200%) to give (3Z)-5-bromo-3-(dimethylaminomethylene)-4-fluoro-isobenzofuran-1-one (850 mg, 68.6% yield) as a yellow solid. ¹H NMR (400 MHz, DMSO-d₆) δ=7.51 (d, J=8.0 Hz, 1H), 7.47-7.37 (m, 1H), 6.67 (s, 1H), 3.17 (s, 6H).

Step 6: A mixture of (3Z)-5-bromo-3-(dimethylaminomethylene)-4-fluoro-isobenzofuran-1-one (2.85 g, 9.96 mmol, 1.00 eq.) and hydrazine hydrate (1.27 g, 24.9 mmol, 1.24 mL, 98.0% purity, 2.50 eq.) in ethyl alcohol (70.0 mL) was stirred at 80° C. for 3 hours under nitrogen. The mixture was concentrated to remove ethyl alcohol. The residue was purified by column chromatography (silica gel, Ethyl acetate/Petroleum ether 2-100% then Methyl 44S alcohol/Dichloromethane 1-10%) to give 6-bromo-4-[(dimethylamino)methyl]-5-fluoro-2H-phthalazin-1-one (900 mg, 30.1% yield) as a brown solid. LCMS [M+1]⁺: 302.0

Step 7: To a mixture of 6-bromo-4-[(dimethylamino)methyl]-5-fluoro-2H-phthalazin-1-one (200 mg, 666 μmol, 1.00 eq.) in THF (10.0 mL) was added isobutyl carbonochloridate (182 mg, 1.33 mmol, 175 μL, 2.00 eq.) dropwise at 0° C., the mixture was stirred at 25° C. for 3 hours under nitrogen atmosphere. The mixture was concentrated to remove THF. The residue was purified by prep-TLC (silica gel plate, Petroleum ether/Ethyl acetate 3:1) to give 6-bromo-4-(chloromethyl)-5-fluoro-2H-phthalazin-1-one (50.0 mg, 25.7% yield) as a yellow solid. ¹H NMR (400 MHz, MeOD) δ=10.21-10.04 (m, 1H), 8.27-8.14 (m, 11H), 8.08-7.98 (m, 1H), 4.90-4.86 (m, 2H).

Step 8: A mixture of 6-bromo-4-(chloromethyl)-5-fluoro-2H-phthalazin-1-one (50.0 mg, 172 μmol, 1.00 eq.) and ammonium hydroxide (455 mg, 4.67 mmol, 0.50 mL, 36.0% purity, 27.3 eq.) in N.N-dimethylformamide (2.0 mL) was stirred at 25° C. for 12 hours. The mixture was concentrated to remove N.N-dimethylformamide to give 4-(aminomethyl)-6-bromo-5-fluoro-2H-phthalazin-1-one (50.0 mg, crude) as a yellow solid and used into the next step without further purification. LCMS [M+1]⁺: 271.9

Step 9: To a mixture of 4-(aminomethyl)-6-bromo-5-fluoro-2H-phthalazin-1-one (50.0 mg, 184 μmol, 1.00 eq.) and potassium carbonate (50.8 mg, 368 μmol, 2.00 eq.) in N.N-dimethylformamide (2.0 mL) was added di-tert-butyl dicarbonate ester (44.1 mg, 202 μmol, 46.4 μL, 1.10 eq.) at 20° C. and the mixture was stirred at 20° C. for 2 hours. The mixture was concentrated to remove N.N-dimethylformamide. The residue was purified by prep-TLC (silica gel plate, Petroleum ether/Ethyl acetate 2:1) to afford tert-butyl N-[(7-bromo-8-fluoro-4-oxo-3H-phthalazin-1-yl)methyl]carbamate (15.0 mg, 17.5% yield) as a white solid. LCMS [M−56+1]⁺: 317.9

Step 10: A mixture of 2-[2-methyl-4-(4,4,5,5-tetramethyl-1,3,2-dioxaborolan-2-yl)pyrazol-3-yl]naphthalene-1-carbonitrile (77.2 mg, 96.7 μmol, 45.0% purity, 1.20 eq.), Intermediate DS (30.0 mg, 80.6 μmol, 1.00 eq.), Pd(dppf)Cl₂ (5.90 mg, 8.06 μmol, 0.10 eq.) and sodium carbonate (25.6 mg, 242 μmol, 3.00 eq.) in dioxane (4.00 mL) and water (0.30 mL) was stirred at 80° C. for 2 hours under nitrogen atmosphere. The mixture was concentrated to remove dioxane and water. The residue was purified by prep-TLC (silica gel plate, Petroleum ether/Ethyl acetate 1:1) to give 4-methyl-7-morpholino-pyrido[3,4-d] pyridazin-1-ol (10 mg, 35.5% yield) as a white solid. LCMS [M+1]⁺: 525.2

Step 10: To a mixture of tert-butyl N-[[7-[5-(1-cyano-2-naphthyl)-1-methyl-pyrazol-4-yl]-8-fluoro-4-oxo-3H-phthalazin-1-yl]methyl]carbamate (35.0 mg, 40.0 μmol, 60.0% purity, 1.00 eq.) in dichloromethane (0.70 mL) was added TFA (539 mg, 4.73 mmol, 350 μL, 118 eq.) at 20° C., the mixture was stirred at 20° C. for 30 mins. The mixture was concentrated to remove TFA and dichloromethane. The residue was purified by prep-HPLC (column: Phenomenex Luna Cis 150×25 mm×10 μm; mobile phase: [water (0.05% HCl)-ACN]; B %: 12%-42%, 11 min) to give 2-[4-[4-(aminomethyl)-5-fluoro-1-oxo-2H-phthalazin-6-yl]-2-methyl-pyrazol-3-yl]naphthalene-1-carbonitrile (1.79 mg, 9.69/a yield) as a yellow oil. LCMS [M+1]⁺: 425.0. ¹H NMR (400 MHz, CDCl₃) δ=13.05 (s, 1H), 8.48 (d, J=8.8 Hz, 1H), 8.35-8.22 (m, 4H), 8.10 (d, J=8.0 Hz, 1H), 8.05 (d, J=2.4 Hz, 1H), 7.91 (d, J=8.4 Hz, 1H), 7.89-7.79 (m, 3H), 7.42-7.46 (m, 1H), 4.23-4.35 (m, 2H), 3.84 (s, 3H).

### Example 17-3

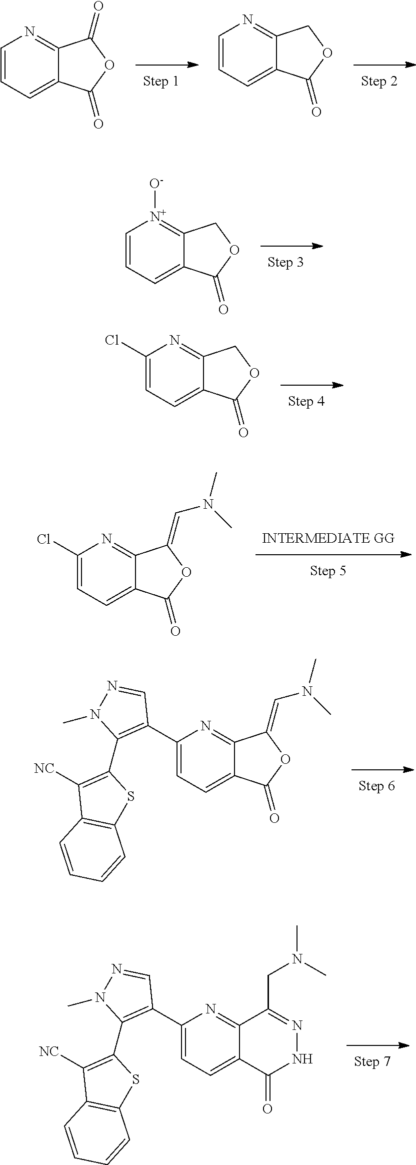

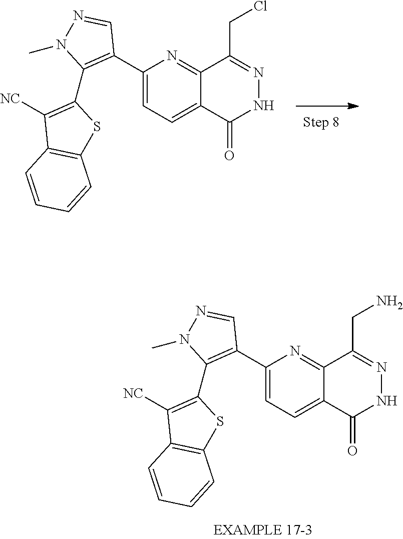 [SMILES: Cn1ncc(-c2ccc3c(=O)[nH]nc(CCl)c3n2)c1-c1sc2ccccc2c1C#N ; Cn1ncc(-c2ccc3c(=O)[nH]nc(CN)c3n2)c1-c1sc2ccccc2c1C#N]

Step 1: Sodium borohydride (5.07 g, 134 mmol, 1.00 eq.) was added to a solution of furo[3,4-b]pyridine-5,7-dione (20.0 g, 134 mmol, 1.00 eq.) in THF (200 mL) at 10° C. under nitrogen followed by acetic acid (16.1 g, 268 mmol, 15.3 mL, 2.00 eq.) dropwise. The mixture was stirred at 10° C. for 4 hours under nitrogen atmosphere. The mixture was concentrated to remove THF before acetic acid (52.5 g, 874 mmol, 50 mL, 6.52 eq.) and acetic anhydride (54.5 g, 534 mmol, 50 mL, 3.98 eq.) were added to the residue and stirred at 100° C. for 3 hours. Saturated ammonium chloride (500 mL) was added to the mixture that was after that stirred at 20° C. for 30 minutes before being extracted with ethyl acetate (500 mL×3). The combined organic phase was washed with brine (100 mL×3), dried over anhydrous sodium sulfate, filtered and concentrated in vacuum. The residue was purified by column chromatography (silica gel, Ethyl acetate/Petroleum ether 5-25%) to give furo[3,4-b]pyridin-5 (7H)-one (17.0 g, 47.6% yield) as a white solid. ¹H NMR (400 MHz, CDCl₃) δ=8.89 (dd, J=5.6, 1.6 Hz, 1H), 8.24 (dd, J=8.0, 1.6 Hz, 1H), 7.53-7.50 (m, 1H), 5.36 (s, 2H).

Step 2: To a solution of furo[3,4-b]pyridin-5 (7H)-one (8.00 g, 59.2 mmol, 1.00 eq.) in dichloromethane (200 mL) was added 3-chlorobenzenecarboperoxoic acid (18.0 g, 88.8 mmol, 85.0% purity, 1.50 eq.) at 0° C. The solution was stirred at 0° C. for 12 hours. The reaction mixture was quenched by addition saturated sodium sulfite (80 mL), diluted with water (200 mL) and extracted with dichloromethane/methanol (10:1) (200 mL×3). The combined organic layers were washed with brine (50 mL×3), dried over anhydrous sodium sulfate, filtered and concentrated under reduced pressure and the residue was purified by column chromatography (silica gel, Methanol/Dichloromethane 1-10%) to give 5-oxo-5,7-dihydrofuro[3,4-b]pyridine 1-oxide (5.00 g, 55.9% yield) as a white solid. ¹H NMR (400 MHz, DMSO-d₆) δ=8.59 (d, J=6.4 Hz, 1H), 7.83-7.81 (m, 1H), 7.65 (t, J=7.2 Hz, 1H), 5.42 (s, 2H).

Step 3: A mixture of 5-oxo-5,7-dihydrofuro[3,4-b]pyridine 1-oxide (5.00 g, 33.1 mmol, 1.00 eq.) in phosphoryl trichloride (29.7 g, 194 mmol, 18.0 mL, 5.85 eq.) was stirred at 110° C. for 10 minutes under nitrogen atmosphere. The reaction mixture was quenched by addition of saturated aqueous sodium bicarbonate (50 mL) at 25° C., then pH was adjusted to 7 by 1 M sodium hydroxide aqueous solution (30 mL) and saturated sodium bicarbonate aqueous solution (50 mL). The mixture was filtered and extracted with ethyl acetate (100 mL×3). The combined organic layers were washed with brine (30 mL×3), dried over anhydrous sodium sulfate, filtered and concentrated under reduced pressure and the residue was purified by column chromatography (silica gel, Ethyl acetate/Petroleum ether 2-25%) to give 2-chlorofuro[3,4-b]pyridin-5 (7H)-one (1.00 g, 17.8% yield) as a white solid. ¹H NMR (400 MHz, CDCl₃) δ=8.16 (d, J=8.4 Hz, 1H), 7.53 (d, J=8.0 Hz, 1H), 5.32 (s, 2H).

Step 4: A mixture of 2-chlorofuro[3,4-b]pyridin-5 (7H)-one (500 mg, 2.95 mmol, 1.00 eq.) and potassium tert-butoxide (1.00 M, 1.47 mL, 0.50 eq.) in 1,1-dimethoxy-N,N-dimethyl-methanamine (5.38 g, 45.2 mmol, 6.0 mL, 15.3 eq.) was stirred at 100° C. for 12 hours under nitrogen atmosphere. The mixture was diluted with water (20 mL), extracted with ethyl acetate (20 mL×3), washed with brine (8.00 mL×3), dried over anhydrous sodium sulfate, filtered and concentrated under reduced pressure and the residue was purified by column chromatography (silica gel, Ethyl acetate/Petroleum ether 5-30%) give (Z)-2-chloro-7-((dimethylamino)methylene)furo[3,4-b]pyridin-5 (7H)-one 200 mg (crude) as a yellow solid. ¹H NMR (400 MHz, CDCl₃) δ=7.98 (d, J=8.0 Hz, 1H), 7.05 (d, J=8.0 Hz, 1H), 6.82 (s, 1H), 3.23 (s, 6H).

Step 5: To a solution of (Z)-2-chloro-7-((dimethylamino)methylene)furo[3,4-b]pyridin-5 (7H)-one (140 mg, 623 μmol, 1.00 eq.) and Intermediate GG (367 mg, 623 μmol, 62.0% purity, 1.00 eq.) in 1,4-dioxane (4.50 mL) and water (1.50 mL) were added sodium bicarbonate (157 mg, 1.87 mmol, 72.7 μL, 3.00 eq.) and (Ad₂n-BuP)—Pd G3 (45.4 mg, 62.3 μmol, 0.10 eq.) at 20° C. The solution was degassed and stirred at 90° C. for 2 hours under nitrogen atmosphere. The solution was concentrated under reduced pressure to remove 1,4-dioxane and water and the residue was purified by prep-TLC (silica gel plate, Ethyl acetate/Petroleum ether 2:1) to give (Z)-2-(4-(7-((dimethylamino)methylene)-5-oxo-5,7-dihydrofuro[3,4-b]pyridin-2-yl)-1-methyl-1H-pyrazol-5-yl)benzo[b]thiophene-3-carbonitrile (77.0 mg, 8.96% yield, 31.0% purity) as a yellow solid. LCMS [M+1]⁺: 428.3.

Step 6: To a solution of (Z)-2-(4-(7-((dimethylamino)methylene)-5-oxo-5,7-dihydrofuro[3,4-b]pyridin-2-yl)-1-methyl-1H-pyrazol-5-yl)benzo[b]thiophene-3-carbonitrile (200 mg, 468 μmol, 1.00 eq.) in ethanol (5.0 mL) was added hydrazine hydrate (47.8 mg, 936 μmol, 46.4 μL, 98.0% purity, 2.00 eq.) at 25° C. under nitrogen. The solution was stirred at 90° C. for 1 hour under nitrogen. The solution was diluted with water (8.00 mL), extracted with ethyl acetate (8.00 mL×3), washed with brine (3.0 mL×3), dried over anhydrous sodium sulfate, filtered and concentrated under reduced pressure and the residue was purified by prep-TLC (silica gel, dichloromethane/methanol 10:1) to obtain 2-(4-(8-((dimethylamino) metH-yl)-5-oxo-5,6-dihydropyrido[2,3-d]pyridazin-2-yl)-1-methyl-11H-pyrazol-5-yl)benzo[b]thiopH-ene-3-carbonitrile (20.0 mg, 9.68% yield) as a white solid. ¹H NMR (400 MHz, CDCl₃) δ=10.1 (s, 1H), 8.62 (d, J=8.4 Hz, 1H), 8.21 (s, 1H), 8.08 (d, J=7.2 Hz, 1H), 7.99 (d, J=7.2 Hz, 1H), 7.84 (d, J=8.8 Hz, 1H), 7.67-7.64 (m, 2H), 3.97 (s, 3H), 3.10 (s, 2H), 1.95 (s, 6H).

Step 7: A mixture of 2-(4-(8-((dimethylamino)methyl)-5-oxo-5,6-dihydropyrido[2,3-d]pyridazin-2-yl)-1-methyl-11H-pyrazol-5-yl)benzo[b]thiophene-3-carbonitrile (20.0 mg, 45.3 μmol, 1.00 eq.) and isobutyl carbonochloridate (61.9 mg, 453 μmol, 59.5 μL, 10.0 eq.) in toluene (1.0 mL) was stirred at 75° C. for 12 hours under nitrogen. The solution was concentrated under reduced pressure to remove toluene and the residue was purified by prep-TLC (silica gel plate, petroleum ether/ethyl acetate 2:3) to give 2-(4-(8-(chloromethyl)-5-oxo-5,6-dihydropyrido[2,3-d]pyridazin-2-yl)-1-methyl-1H-pyrazol-5-yl)be-nzo[b]thiophene-3-carbonitrile (15.0 mg) as a white solid. LCMS [M+1]⁺: 433.3

Step 8: A mixture of 2-(4-(8-(chloromethyl)-5-oxo-5,6-dihydropyrido[2,3-d]pyridazin-2-yl)-1-methyl-1H-pyrazol-5-yl)benzo[b]thiophene-3-carbonitrile (15.0 mg, 34.6 μmol, 1.00 eq.) and ammonium hydroxide (273 mg, 2.34 mmol, 0.30 mL, 30.0% purity, 67.4 eq.) in N,N-dimethylformamide (1.0 mL) was stirred at 20° C. for 1 hour. The solution was concentrated under reduced pressure. The residue was purified by prep-HPLC (column: Welch Xtimate C18 150×25 mm×5 μm; mobile phase: [water (0.05% HCl)-ACN]; B %: 4/0-34%, 10 min) to give 2-[4-[8-(aminomethyl)-5-oxo-6H-pyrido[2,3-d]pyridazin-2-yl]-2-methyl-pyrazol-3-yl]benzothiophene-3-carbonitrile (1.01 mg, 6.39% yield) as a white solid. LCMS [M+1]⁺: 414.3. ¹H NMR (400 MHz, MeOD) δ=8.61 (d, J=8.4 Hz, 1H), 8.42 (s, 1H), 8.19-8.15 (m, 1H), 8.08-8.06 (m, 1H), 8.03 (d, J=8.4 Hz, 1H), 7.72-7.68 (m, 2H), 3.96 (s, 3H), 3.82 (s, 2H).

### Example 17-4

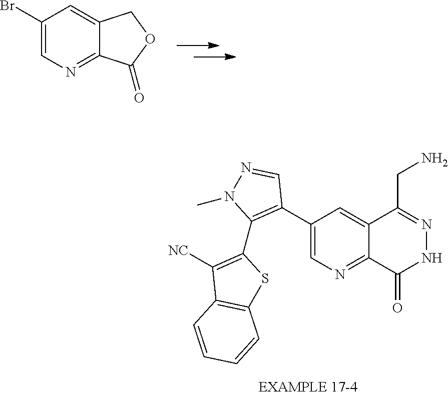 [SMILES: O=C1OCc2cc(Br)cnc21 ; Cn1ncc(-c2cnc3c(=O)[nH]nc(CN)c3c2)c1-c1sc2ccccc2c1C#N]

Example 17-4 is prepared using similar route as example 17-3 starting from 3-bromofuro[3,4-b]pyridin-7 (5H)-one to afford 2-(4-(5-(aminomethyl)-8-oxo-7,8-dihydropyrido[2,3-d]pyridazin-3-yl)-1-methyl-1H-pyrazol-5-yl)benzo[b]thiophene-3-carbonitrile (40 mg, 105 uMmol, HCl salt) as a yellow salt using final purification method 4-25. LCMS [M+1]⁺414.1; ¹H NMR (400 MHz, DMSO-d₆) δ=13.14 (s, 1H), 8.64 (d, J=2.0 Hz, 1H), 8.52 (br d, J=2.0 Hz, 4H), 8.43 (s, 1H), 8.33-8.25 (m, 1H), 8.05-7.96 (m, 1H), 7.73-7.64 (m, 2H), 4.41 (q, J=5.6 Hz, 2H), 3.98 (s, 3H).

### Example 17-6

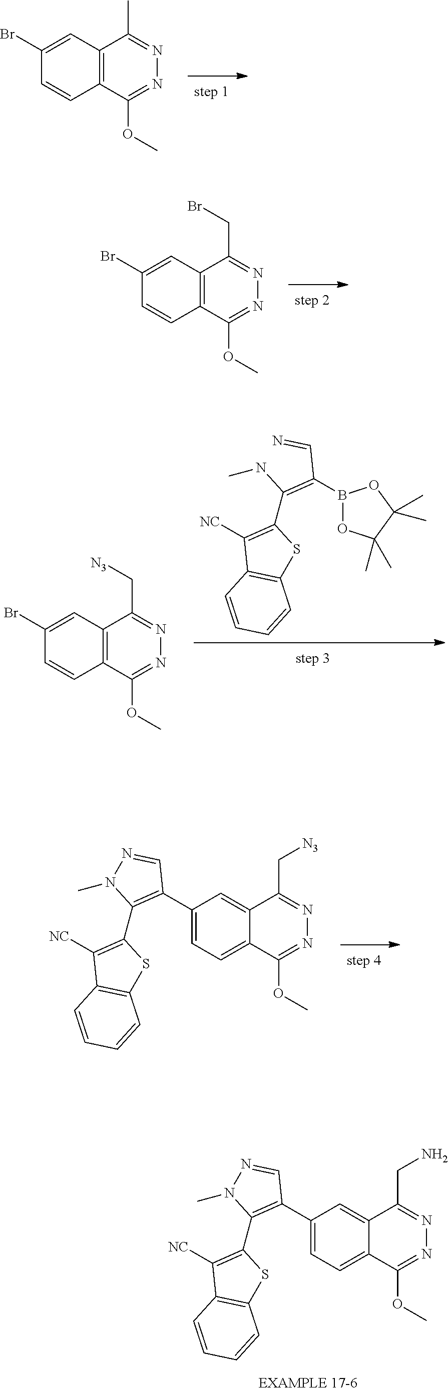 [SMILES: COc1nnc(C)c2cc(Br)ccc12 ; COc1nnc(CBr)c2cc(Br)ccc12 ; COc1nnc(CN=[N+]=[N-])c2cc(Br)ccc12 ; CN/C(=C(\C=N)B1OC(C)(C)C(C)(C)O1)c1sc2ccccc2c1C#N ; COc1nnc(CN=[N+]=[N-])c2cc(-c3cnn(C)c3-c3sc4ccccc4c3C#N)ccc12 ; COc1nnc(CN)c2cc(-c3cnn(C)c3-c3sc4ccccc4c3C#N)ccc12]

Step 1: A mixture of 6-bromo-1-methoxy-4-methyl-phthalazine (370 mg, 1.46 mmol, 1.00 eq.), 1-bromopyrrolidine-2,5-dione (260 mg, 1.46 mmol, 1.00 eq.), 2-(2-cyanopropan-2-yldiazenyl)-2-methylpropanenitrile (24.0 mg, 146 μmol, 0.10 eq.) and in chloroform (12 mL) was degassed and purged with nitrogen for 3 times, and then the reaction mixture was stirred at 70° C. for 3 hours. The mixture was concentrated and the residue diluted with water (100 mL), and extracted with EtOAc (120 mL), washed with brine (100 mL), dried over sodium sulfate, filtered and concentrated under reduced pressure. The residue was purified by flash silica gel chromatography (4 g SepaFlash Silica Flash Column, Eluent of 0-25% EtOAc/Petroleum ether gradient 25 mL/min) to get 6-bromo-4-(bromomethyl)-1-methoxy-phthalazine (233 mg, 684 μmol, 47% yield) as a white solid. ¹H NMR (400 MHz, DMSO-d₆) δ=8.56 (d, J=0.8 Hz, 1H), 8.20-8.14 (m, 2H), 5.25 (s, 2H), 4.19 (s, 3H).

Step 2: To a solution of 6-bromo-4-(bromomethyl)-1-methoxy-phthalazine (233 mg, 702 μmol, 1.00 eq.) in acetonitrile (3 mL) was added sodium azide (68 mg, 1.05 mmol, 1.50 eq.) and the mixture was degassed and purged with nitrogen 3 times and then stirred at 20° C. for 12 hours. The pH of the mixture was then adjusted to pH=10 with an aqueous solution of sodium hydroxide (0.10 N). The mixture was then extracted with tert-butyl methyl ether (60 mL) and the organic phase dried with anhydrous sodium sulfate, filtered and concentrated to give 4-(azidomethyl)-6-bromo-1-methoxy-phthalazine (210 mg, 640 μmol, 91% yield) as a yellow solid. ¹H NMR (400 MHz, DMSO-d₆) δ=8.46 (d, J=1.2 Hz, 1H), 8.20-8.17 (m, 1H), 8.17-8.13 (m, 1H), 5.05 (s, 2H), 4.20 (s, 3H).

Step 3: A mixture of 4-(azidomethyl)-6-bromo-1-methoxy-phthalazine (210 mg, 714 μmol, 1.00 eq.), 2-[2-methyl-4-(4,4,5,5-tetramethyl-1,3,2-dioxaborolan-2-yl)pyrazol-3-yl]benzothiophene-3-carbonitrile (652 mg, 1.07 mmol), [2-(2-aminophenyl)phenyl]palladium(1+);bis(1-adamantyl)-butyl-phosphane;methanesulfonate (104 mg, 143 μmol, 0.20 eq.) and sodium carbonate (227 mg, 2.14 mmol, 3.00 eq.) in dioxane (5 mL) and water (1 mL) was degassed and purged with nitrogen for 3 times, and then the reaction mixture was stirred at 80° C. for 3 hours. The reaction mixture was diluted with water (50 mL) and extracted with EtOAc (120 mL). The organic layers were washed with brine 100 mL, dried over sodium sulfate, filtered, and concentrated under reduced pressure. The residue was purified by prep-TLC (SiO₂, EtOAc:petroleum ether=1:1) to give 4-(azidomethyl)-6-[5-(3-isocyanobenzothiophen-2-yl)-1-methyl-pyrazol-4-yl]-1-methoxy-phthalazine (140 mg, 309 μmol, 39% yield) as a yellow solid. LCMS [M+1]⁺ 453.1.

Step 4: To a solution of 4-(azidomethyl)-6-[5-(3-isocyanobenzothiophen-2-yl)-1-methyl-pyrazol-4-yl]-1-methoxy-phthalazine (15 mg, 33 μmol, 1.00 eq.) in THF (2 mL) was added dry palladium 10% on carbon (20 mg). The mixture was stirred at 25° C. for 3 hours under hydrogen atmosphere. The mixture was filtered and concentrated in vacuum to get a residue. The residue was purified by prep-HPLC (column: Phenomenex Gemini 150×25 mm×10 μm; mobile phase: [water (0.05% NH₃H₂O)-ACN]; B %: 28%-58%, 10 min) to get [7-[5-(3-isocyanobenzothiophen-2-yl)-1-methyl-pyrazol-4-yl]-4-methoxy-phthalazin-1-yl]methanamine (5.27 mg, 11.7 μmol, 35% yield) as a white solid. ¹H NMR (400 MHz, MeOD-d₄) δ=8.19 (t, J=4.0 Hz, 2H), 8.15-8.11 (m, 1H), 8.00 (s, 2H), 7.87-7.82 (m, 1H), 7.71-7.63 (m, 2H), 4.28-4.10 (m, 5H), 4.01 (s, 3H).

### Example 17-7

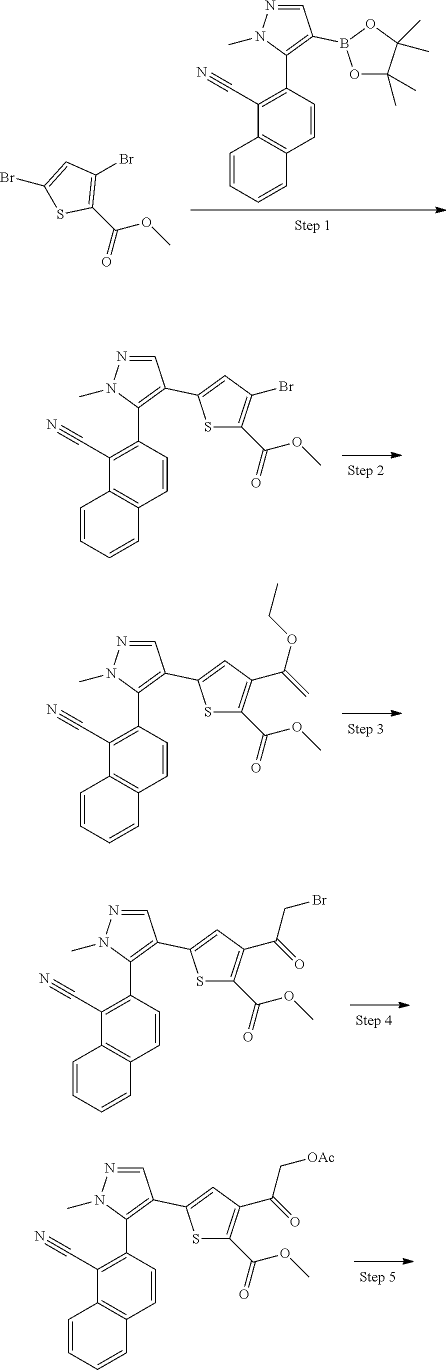 [SMILES: COC(=O)c1sc(Br)cc1Br ; Cn1ncc(B2OC(C)(C)C(C)(C)O2)c1-c1ccc2ccccc2c1C#N ; COC(=O)c1sc(-c2cnn(C)c2-c2ccc3ccccc3c2C#N)cc1Br ; C=C(OCC)c1cc(-c2cnn(C)c2-c2ccc3ccccc3c2C#N)sc1C(=O)OC ; COC(=O)c1sc(-c2cnn(C)c2-c2ccc3ccccc3c2C#N)cc1C(=O)CBr ; COC(=O)c1sc(-c2cnn(C)c2-c2ccc3ccccc3c2C#N)cc1C(=O)COC(C)=O]

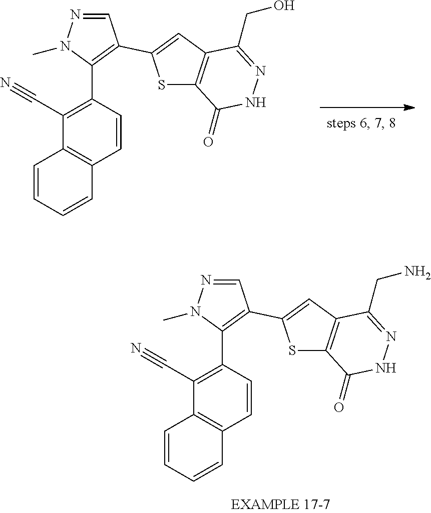 [SMILES: Cn1ncc(-c2cc3c(CO)n[nH]c(=O)c3s2)c1-c1ccc2ccccc2c1C#N ; Cn1ncc(-c2cc3c(CN)n[nH]c(=O)c3s2)c1-c1ccc2ccccc2c1C#N]

Step 1: A mixture of methyl 3,5-dibromothiophene-2-carboxylate (500 mg, 1.67 mmol, 1.20 eq.), 2-[2-methyl-4-(4,4,5,5-tetramethyl-1,3,2-dioxaborolan-2-yl)pyrazol-3-yl]naphthalene-1-carbonitrile (1.00 g, 1.39 mmol, 50% purity, 1.00 eq.), tetrakis[triphenylphosphine]palladium (161 mg, 139 umol, 0.1 eq.), potassium carbonate (383.70 mg, 2.78 mmol, 1.99 eq.) and water (1.00 mL) in toluene (10 mL) was degassed and purged with nitrogen for 3 times, and then the mixture was stirred at 100° C. for 12 hours under nitrogen atmosphere. After being cooled to 20° C., the mixture was concentrated under vacuum. The residue was diluted with ethyl acetate (20.0 mL) and water (20.0 mL). The layers were separated and the aqueous phase was extracted with ethyl acetate (3×20.0 mL). The organic layer was dried over Na₂SO₄, filtered and concentrated under vacuum. The crude product was purified by reversed-phase HPLC (0.1% TFA condition). The crude product is later purified by flash silica gel chromatography (ISCO®; 20 g Sep a Flash® Silica Flash Column, Eluent of 0-40/o Ethyl acetate/Petroleum ether gradient @ 100 mL/min) to give methyl 3-bromo-5-[5-(1-cyano-2-naphthyl)-1-methyl-pyrazol-4-yl]thiophene-2-carboxylate (450 mg, 995 umol, 71.5% yield) as a yellow solid. ¹H NMR (400 MHz, CDCl₃-d) δ=8.35 (d, J=8.4 Hz, 1H), 8.24 (d, J=8.4 Hz, 1H), 8.06 (d, J=8.0 Hz, 1H), 7.88-7.74 (m, 31H), 7.50 (d, J=8.4 Hz, 1H), 6.79 (s, 1H), 3.78-3.77 (m, 6H).

Step 2: A mixture of methyl 3-bromo-5-[5-(1-cyano-2-naphthyl)-1-methyl-pyrazol-4-yl]thiophene-2-carboxylate (450 mg, 995 umol, 1.00 eq.), tributyl(1-ethoxyvinyl) stannane (430 mg, 1.19 mmol, 401 uL, 1.20 eq.), bis(triphenylphosphine) palladium(II) chloride (69.8 mg, 99.5 umol, 0.10 eq.) in dioxane (10.0 mL) was degassed and purged with nitrogen for 3 times, and then the mixture was stirred at 80° C. for 12 hours under nitrogen atmosphere. The reaction mixture was diluted with ethyl acetate (20.0 mL) and very slowly added to saturated aqueous potassium fluoride (20.0 mL). The mixture was extracted with ethyl acetate (3×20.0 mL). The organic layer was dried over sodium sulfate, filtered and concentrated under vacuum. The residue was purified by flash silica gel chromatography (ISCO®; 20 g SepaFlash® Silica Flash Column, Eluent of 0˜30% Ethyl acetate/Petroleum ether gradient @ 80 mL/min). methyl5-[5-(1-cyano-2-naphthyl)-1-methyl-pyrazol-4-yl]-3-(1-ethoxyvinyl) thiophene-2-carboxylate (390 mg, 879 umol, 88.4% yield) as a yellow solid. ¹H NMR (400 MHz, CDCl₃-d) δ=8.34 (d, J=8.4 Hz, 1H), 8.23 (d, J=8.4 Hz, 1H), 8.05 (d, J=8.0 Hz, 1H), 7.86 (s, 1H), 7.85-7.80 (m, 1H), 7.79-7.73 (m, 1H), 7.50 (d, J=8.4 Hz, 1H), 6.81 (s, 1H), 3.78-3.76 (m, 6H), 3.71 (q, J=7.2 Hz, 2H), 1.29-1.19 (m, 3H).

Step 3: To a solution of methyl 5-[5-(1-cyano-2-naphthyl)-1-methyl-pyrazol-4-yl]-3-(1-ethoxyvinyl)thiophene-2-carboxylate (390 mg, 879 umol, 1.00 eq.) in tetrahydrofuran (5.00 mL) was added 1-bromopyrrolidine-2,5-dione (170 mg, 955 umol, 1.09 eq.) and water (1.20 mL). The mixture was stirred at 20° C. for 1 hour. The residue was diluted with ethyl acetate (20.0 mL) and water (20.0 mL). The layers were separated and the aqueous phase was extracted with ethyl acetate (3×20.0 mL). The organic layer was dried over anhydrous sodium sulfate, filtered and concentrated under vacuum. The residue was purified by column chromatography on silica gel (Petroleum ether:Ethyl acetate=1/1) to give methyl3-(2-bromoacetyl)-5-[5-(1-cyano-2-naphthyl)-1-methyl-pyrazol-4-yl] thiophene-2-carboxylate (100 mg, 185 umol, 20.9% yield, 91.0/6 purity) as a yellow solid. ¹H NMR (400 MHz, CDCl₃-d) δ=8.33-8.32 (m, 1H), 8.24 (d, J=8.4 Hz, 1H), 8.07-7.99 (m, 2H), 7.88 (s, 1H), 7.84-7.76 (m, 2H), 7.50 (d, J=8.4 Hz, 1H), 6.80 (s, 1H), 4.36 (s, 2H), 3.80-3.78 (m, 6H).

Step 4: To a solution of methyl 3-(2-bromoacetyl)-5-[5-(1-cyano-2-naphthyl)-1-methyl-pyrazol-4-yl] thiophene-2-carboxylate (80.0 mg, 162 umol, 100 eq.) in dimethylformamide (3.00 mL) was added sodium acetate (26.6 mg, 324 umol, 2.00 eq.). The mixture was stirred at 80° C. for 2 hours. After being cooled to room temperature, the mixture was diluted with ethyl acetate (20.0 mL) and water (20.0 mL). The layers were separated and the aqueous phase was extracted with ethyl acetate (3×15.0 mL). The organic layer was dried over anhydrous sodium sulfate, filtered and concentrated under vacuum to give methyl 3-(2-acetoxyacetyl)-5-[5-(1-cyano-2-naphthyl)-1-methyl-pyrazol-4-yl]thiophene-2-carboxylate (80 mg, crude) as a yellow solid. ¹H NMR (400 MHz, CDCl₃-d) δ=8.34 (d, J=8.4 Hz, 1H), 8.24 (d, J=8.4 Hz, 1H), 8.05 (d, J=8.0 Hz, 1H), 7.87 (s, 1H), 7.85-7.77 (m, 2H), 7.49 (d, J=8.4 Hz, 1H), 6.88 (s, 1H), 5.03 (s, 2H), 3.79-3.77 (m, 6H), 2.09 (s, 3H).

Step 5: To a solution of methyl 3-(2-acetoxyacetyl)-5-[5-(1-cyano-2-naphthyl)-1-methyl-pyrazol-4-yl] thiophene-2-carboxylate (80.0 mg, 169 umol, 1.00 eq.) in ethanol (5.00 mL) was added hydrazine hydrate (169 mg, 3.31 mmol, 164 uL, 98% purity, 19.6 eq.). The mixture was stirred at 70° C. for 2 hours. After being cooled to room temperature, the mixture was concentrated under vacuum. The residue was purified by column chromatography on silica gel (dichloromethane:methanol=20/1) to give 2-[4-[4-(hydroxymethyl)-7-oxo-6H-thieno [2, 3-d] pyridazin-2-yl]-2-methyl-pyrazol-3-yl] naphthalene-1-carbonitrile (50 mg, 121 umol, 71.6% yield) as a yellow solid. ¹H NMR (400 MHz, DMSO-d₆) δ=12.65 (s, 1H), 8.58 (d, J=8.4 Hz, 1H), 8.31 (d, J=7.6 Hz, 1H), 8.28 (s, 1H), 8.21 (d, J=8.4 Hz, 1H), 7.93 (dd, J=1.2, 8.4 Hz, 1H), 7.91-7.85 (m, 2H), 7.75 (s, 1H), 5.42 (t, J=6.0 Hz, 1H), 4.54 (d, J=6.0 Hz, 2H), 3.75 (s, 3H).

Step 6: To a solution of 2-[4-[4-(hydroxymethyl)-7-oxo-6H-thieno[2,3-d]pyridazin-2-yl]-2-methyl-pyrazol-3-yl]naphthalene-1-carbonitrile (50 mg, 121 umol, 1.00 eq.) in thionyl chloride (1.64 g, 13.8 mmol, 1.00 mL, 114 eq.) was added dimethylformamide (442 ug, 6.05 umol, 0.05 eq.). The mixture was stirred at 15° C. for 10 minutes. The reaction mixture was diluted with dichloromethane (10.0 mL) and very slowly added to saturated aqueous sodium bicarbonate (10.0 mL). The mixture was extracted with dichloromethane (3×10.0 mL). The organic layer was dried over anhydrous sodium sulfate, filtered and concentrated under vacuum to give 2-[4-[4-(chloromethyl)-7-oxo-6H-thieno[2,3-d]pyridazin-2-yl]-2-methyl-pyrazol-3-yl]naphthalene-1-carbonitrile (50.0 mg, 101 umol, 84.2% yield, 88.0% purity) as a yellow solid. ¹H NMR (400 MHz, DMSO-d₆) δ=12.91 (s, 1H), 8.58 (d, J=8.4 Hz, 1H), 8.33-8.28 (m, 2H), 8.21 (d, J=8.4 Hz, 1H), 7.96-7.84 (m, 3H), 7.68 (s, 1H), 4.90-4.77 (m, 2H), 3.77 (s, 3H).

Step 7: To a solution of 2-[4-[4-(chloromethyl)-7-oxo-6H-thieno[2,3-d]pyridazin-2-yl]-2-methyl-pyrazol-3-yl]naphthalene-1-carbonitrile (50.0 mg, 115 umol, 1.00 eq.) in dimethylformamide (5.00 mL) was added (1,3-dioxoisoindolin-2-yl)potassium (25.73 mg, 138.92 umol, 1.20 eq.). The mixture was stirred at 60° C. for 2 hours. After being cooled to room temperature, the mixture was concentrated in vacuum. The residue was purified by column chromatography on silica gel (dichloromethane/methanol 20:1) to give 2-[4-[4-[(1,3-dioxoisoindolin-2-yl)methyl]-7-oxo-6H-thieno[2,3-d]pyridazin-2-yl]-2-methyl-pyrazol-3-yl]naphthalene-1-carbonitrile (25 mg, 46.1 umol, 39.8% yield) as a yellow solid. ¹H NMR (400 MHz, DMSO-d₆) δ=12.63 (s, 1H), 8.59 (d, J=8.4 Hz, 1H), 8.34-8.28 (m, 2H), 8.24 (d, J=8.4 Hz, 1H), 7.95-7.87 (m, 7H), 7.84 (s, 1H), 5.03 (s, 2H), 3.78 (s, 3H).

Step 8: To a solution of 2-[4-[4-[(1,3-dioxoisoindolin-2-yl)methyl]-7-oxo-6H-thieno[2,3-d]pyridazin-2-yl]-2-methyl-pyrazol-3-yl]naphthalene-1-carbonitrile (25.0 mg, 46.1 umol, 1.00 eq.) in ethanol (5.00 mL) was added hydrazine hydrate (46.1 mg, 903 umol, 44.8 uL, 98% purity, 19.6 eq.). The mixture was stirred at 50° C. for 1 hour. After being cooled to room temperature, the mixture was concentrated under vacuum. The residue was purified by prep-HPLC (column: Welch Xtimate C18 150×25 mm×5 um; mobile phase: [water (0.05% HCl)-ACN]; B %: 10%-40%, 10 min) to give 2-(4-(4-(aminomethyl)-7-oxo-6, 7-dihydrothieno[2,3-d]pyridazin-2-yl)-1-methyl-1H-pyrazol-5-yl)-1-naphthonitrile (8.6 mg, 18.6 umol, 40.3% yield, 97% purity, HCl salt) as a yellow solid. ¹H NMR (400 MHz, MeOD-d₄) δ=8.48 (d, J=8.4 Hz, 1H), 8.27 (d, J=8.4 Hz, 1H), 8.24-8.17 (m, 2H), 7.92-7.82 (m, 2H), 7.77-7.72 (m, 1H), 7.69 (s, 1H), 4.45-4.57 (m, 2H), 3.81 (s, 3H).

### Example 18-1

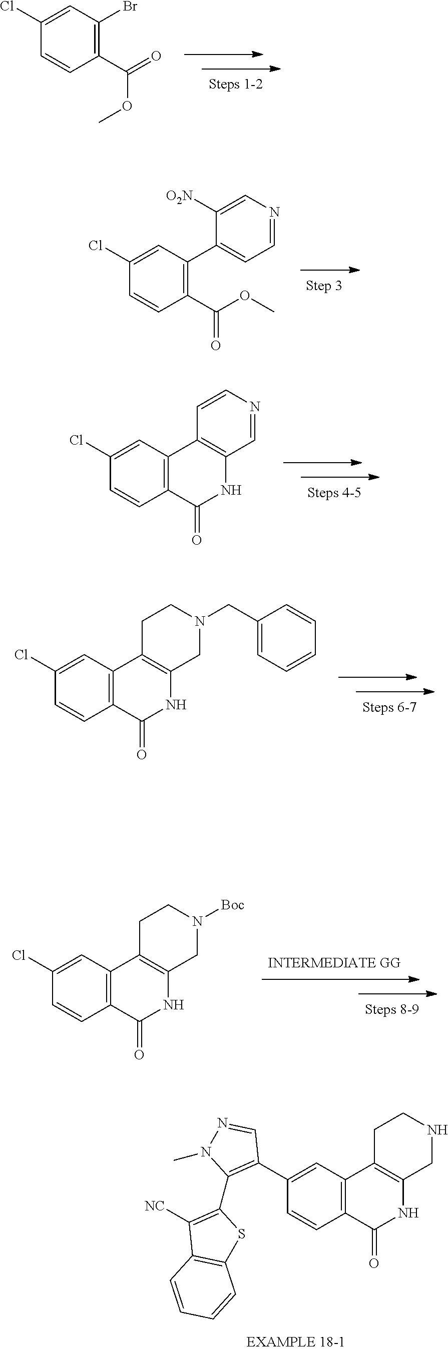

Step 1: A mixture of methyl 2-bromo-4-chloro-benzoate (1.00 g, 4.01 mmol, 1.00 eq.), Pd(dppf)Cl₂ (293 mg, 401 μmol, 0.10 eq.), 4,4,5,5-tetramethyl-2-(4,4,5,5-tetramethyl-1,3,2-dioxaborolan-2-yl)-1,3,2-dioxaborolane (1.22 g, 4.81 mmol, 1.20 eq.) and potassium acetate (1.18 g, 12.0 mmol, 3.00 eq.) in dioxane (15 mL) was degassed and stirred at 85° C. for 10 hours. The reaction mixture was concentrated under vacuum to get the residue. The residue was purified by silica gel column chromatography (Ethyl acetate/Petroleum ether 5-50%) to give methyl 4-chloro-2-(4,4,5,5-tetramethyl-1,3,2-dioxaborolan-2-yl)benzoate (360 mg, 30.2% yield) as white solid. ¹H NMR (400 MHz, CDCl₃-d) δ=7.88 (d, J=8.4 Hz, 1H), 7.45 (d, J=2.0 Hz, 1H), 7.39 (dd, J=2.0, 8.4 Hz, 1H), 3.92 (s, 3H), 1.43 (s, 12H).

Step 2: A mixture of methyl 4-chloro-2-(4,4,5,5-tetramethyl-1,3,2-dioxaborolan-2-yl)benzoate (3.20 g, 10.8 mmol, 1.00 eq.), 4-chloro-3-nitropyridine (1.88 g, 11.9 mmol, 1.10 eq.), Pd(dppf)Cl₂ (790 mg, 1.08 mmol, 0.10 eq.), potassium carbonate (3.73 g, 27.0 mmol, 2.50 eq.) in dioxane (20 mL) and water (4.00 mL) was degassed and stirred at 80° C. for 12 hours under nitrogen atmosphere. The reaction mixture was poured into water (30 mL). The mixture was extracted with ethyl acetate (50 mL×3). The organic phase was washed with brine (20 mL), dried over anhydrous sodium sulfate, and concentrated in vacuum and the residue was purified by flash silica gel chromatography (Ethyl acetate/Petroleum ether 0-10%) to give methyl 4-chloro-2-(3-nitro-4-pyridyl)benzoate (690 mg, 21.9% yield) as a white solid. LCMS [M+1]⁺=293.1.

Step 3: To a mixture of methyl 4-chloro-2-(3-nitro-4-pyridyl)benzoate (800 mg, 2.73 mmol, 1.00 eq.) and ammonium chloride (877 mg, 16.4 mmol, 6.00 eq.) in ethyl alcohol (15 mL) were added water (3.0 mL) and iron (763 mg, 13.7 mmol, 5.00 eq.). The reaction mixture was stirred at 70° C. for 3 hours. The reaction mixture was filtered and the filter cake was washed with methanol (100 mL×4). The filtrate was concentrated under vacuum to give a crude product. The crude product was triturated in water (30 mL) and then washed with water (30 mL). The solid was dried under vacuum to give 9-chloro-5H-benzo[c][1,7]naphthyridin-6-one (360 mg, 57.10% yield) as white solid. ¹H NMR (400 MHz, DMSO-d₆) δ=12.31-11.50 (m, 1H), 8.87-8.04 (m, 5H), 7.95-7.50 (m, 1H).

Step 4: To a mixture of 9-chloro-5H-benzo[c][1,7]naphthyridin-6-one (360 mg, 1.56 mmol, 1.00 eq.) in dimethyl formamide (6.0 mL) was added benzyl bromide (267 mg, 1.56 mmol, 185 μL, 1.00 eq.). The reaction mixture was stirred at 80° C. for 16 hours. The reaction mixture was filtered and the solid was washed with water (30 mL). The solid was dried under vacuum to give 3-benzyl-9-chloro-5H-benzo[c][1,7]naphthyridin-3-ium-6-one bromide (423 mg, 67.5% yield) as a white solid. LCMS [M+1]⁺=321.0. ¹H NMR (400 MHz, DMSO-d₆) δ=12.85-12.09 (m, 1H), 9.47-8.70 (m, 4H), 8.40 (br s, 1H), 8.06 (br s, 1H), 7.72-6.85 (m, 5H), 6.49-5.30 (m, 2H).

Step 5: To a mixture of 3-benzyl-9-chloro-5H-benzo[c][1,7]naphthyridin-3-ium-6-one (100 mg, 249 μmol, 1.00 eq.) in methyl alcohol (10.0 mL) was added sodium borohydride (0.54 g, 14.3 mmol, 57.3 eq.) at 15° C. in 5 portions over 5 hours. The reaction mixture was stirred at 15° C. for 12 hours. The reaction was added into 1N hydrogen chloride (30 mL) with stirring. Then the pH of the mixture was adjusted to 7 with the addition of sodium bicarbonate. The resulting suspension was filtered and the solid was washed with water (30 mL). The solid was dried under vacuum to give 3-benzyl-9-chloro-1,2,4,5-tetrahydrobenzo[c][1,7]naphthyridin-6-one (80.0 mg, 98.9% yield) as a white solid. LCMS [M+1]⁺=325.3. ¹H NMR (400 MHz, DMSO-d₆) δ=11.53-10.78 (m, 1H), 8.46-7.93 (m, 1H), 7.72-7.11 (m, 7H), 6.54-6.52 (m, 1H), 3.81-3.54 (m, 2H), 3.26-3.08 (m, 2H), 2.84-2.66 (m, 4H).

Step 6: To a mixture of 3-benzyl-9-chloro-1,2,4,5-tetrahydrobenzo[c][1,7]naphthyridin-6-one (170 mg, 523 μmol, 1.00 eq.) in anhydrous dichloromethane (15 mL) was added 1-chloroethyl carbonochloridate (112 mg, 785 μmol, 1.50 eq.). The reaction mixture was stirred at 45° C. for 2 hours. Then the reaction mixture was concentrated under vacuum to produce a residue. Methanol (15 mL) was added and the mixture was heated to 70° C. for 1 hour. The reaction mixture was concentrated under vacuum and the residue was purified by prep-TLC (silica gel plate, Dichloromethane/Methanol 30:1) afford 9-chloro-2,3,4,5-tetrahydro-1H-benzo[c][1,7]naphthyridin-6-one (123 mg, crude) as a white solid. LCMS [M+1]⁺=235.1.

Step 7: To a mixture of 9-chloro-2,3,4,5-tetrahydro-1H-benzo[c][1,7]naphthyridin-6-one (123 mg, 524 μmol, 1 eq.) and triethylamine (159 mg, 1.57 mmol, 219 μL, 3.00 eq.) in dichloromethane (20 mL) was added tert-butyl (2-methylpropan-2-yl)oxycarbonyl carbonate (229 mg, 1.05 mmol, 241 μL, 2.00 eq.). The reaction mixture was stirred at 15° C. for 2 hours. The reaction mixture was concentrated under vacuum to yield a residue. The residue was purified by prep-TLC (dichloromethane/methyl alcohol 20:1) to give tert-butyl 9-chloro-6-oxo-1,2,4,5-tetrahydrobenzo[c][1,7]naphthyridine-3-carboxylate (154 mg, 87.8% yield) as a white solid. ¹H NMR (400 MHz, DMSO-d₆) 5=12.56-12.37 (m, 1H), 9.25 (d, J=8.4 Hz, 1H), 8.72 (d, J=2.0 Hz, 1I H), 8.57 (dd, J=2.0, 8.4 Hz, 1H), 8.45-8.42 (m, 1H), 5.38 (br s, 2H), 4.70 (br t, J=5.6 Hz, 2H), 3.75-3.70 (m, 2H).

Step 8: To a mixture of tert-butyl 9-chloro-6-oxo-1,2,4,5-tetrahydrobenzo[c][1,7]naphthyridine-3-carboxylate (80.0 mg, 239 μmol, 1.00 eq.) and Intermediate GG (175 mg, 478 μmol, 2.00 eq.) in dioxane (4.00 mL) and water (0.50 mL) were added SPhos-Pd-G2 (34.4 mg, 47.8 μmol, 0.20 eq.) and cesium carbonate (156 mg, 478 μmol, 2.00 eq.). The reaction mixture was degassed and heated to 80° C. for 2 hours. Ethyl acetate (25 mL) and water (25 mL) were added and the mixture was separated. The organic layer was dried over sodium sulfate and then concentrated under vacuum to produce a residue. The residue was purified by prep-TLC (Dichloromethane/Methyl alcohol 20:1) to give tert-butyl 9-[5-(3-cyanobenzothiophen-2-yl)-1-methyl-pyrazol-4-yl]-6-oxo-1,2,4,5-tetrahydrobenzo[c][1,7]naphthyridine-3-carboxylate (25.0 mg, 11.3% yield, 58.0/o purity) as a white solid. LCMS [M+1]=537.9

Step 9: To a solution of tert-butyl 9-(5-(3-cyanobenzo[b]thiophen-2-yl)-1-methyl-1H-pyrazol-4-yl)-6-oxo-1,4,5,6-tetrahydrobenzo[c][1,7]naphthyridine-3 (2H)-carboxylate (25.0 mg, 46.5 μmol, 1.00 eq.) in dichloromethane (2.0 mL) was added trifluoroacetic acid (770 mg, 6.75 mmol, 0.50 mL, 145 eq.). The mixture was stirred at 15° C. for 0.5 hour. The reaction mixture was concentrated under reduced pressure and the residue was purified by prep-HPLC (column: Welch Xtimate C18 150×25 mm×5 μm; mobile phase: [water (0.05% HCl)-ACN]; B %: 1%-30%, 10 min) to produce 2-(1-methyl-4-(6-oxo-1,2,3,4,5,6-hexahydrobenzo[c][1,7]naphthyridin-9-yl)-1H-pyrazol-5-yl)benzo[b]thiophene-3-carbonitrile (4.70 mg, 21.9% yield) as a yellow solid. LCMS [M+1]⁺=438.0. ¹H NMR (400 MHz, DMSO-d₆) δ=11.33 (s, 1H), 9.22 (br s, 2H), 8.29 (d, J=6.8 Hz, 1H), 8.23 (s, 1H), 8.09 (d, J=8.4 Hz, 1H), 8.01-7.96 (m, 1H), 7.72-7.65 (m, 2H), 7.43 (s, 1H), 7.37 (d, J=8.0 Hz, 1H), 3.99 (br s, 2H), 3.93 (s, 3H), 3.30-3.24 (m, 2H), 2.54-2.52 (m, 2H).

### Example 18-2

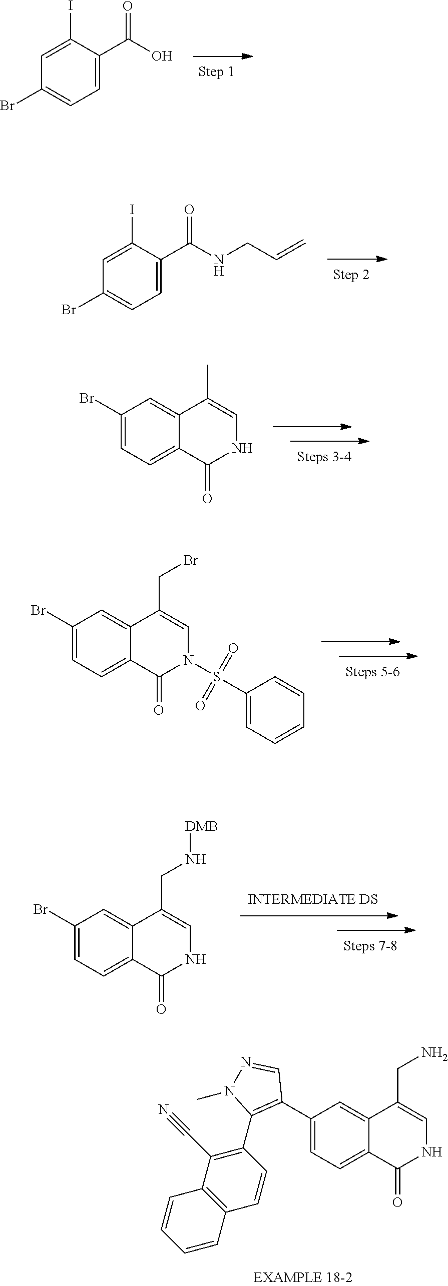

Step 1: To a solution of 4-bromo-2-iodo-benzoic acid (5.00 g, 15.3 mmol, 1.00 eq.) in dimethyl formamide (50 mL) was added HATU (8.72 g, 22.9 mmol, 1.50 eq.) and diisopropylethylamine (5.93 g, 45.9 mmol, 7.99 mL, 3.00 eq.). The mixture was stirred at 10° C. for 0.5 hour. Following that prop-2-en-1-amine (961 mg, 16.8 mmol, 1.26 mL, 1.10 eq.) was added and the resulting mixture was stirred at 10° C. for 11.5 hours. The reaction mixture was diluted with water (200 mL) and extracted with ethyl acetate (150 mL 3). The combined organic layers were washed with brine (200 mL), dried over anhydrous sodium sulfate, filtered and concentrated under reduced pressure and the residue was purified by column chromatography (SiO₂, ethyl acetate/petroleum ether 10-35%) to give N-allyl-4-bromo-2-iodo-benzamide (5.10 g, 91.1% yield) as a yellow solid. LCMS [M+1]⁺=367.9. ¹H NMR (400 MHz, CDCl₃) δ=8.02 (d, J=2.0 Hz, 1H), 7.51 (dd, J=2.0, 8.0 Hz, 1H), 7.25 (d, J=2.4 Hz, 1H), 6.00-5.83 (m, 2H), 5.31 (qd, J=1.2, 17.2 Hz, 1H), 5.21 (dd, J=1.2, 10.0 Hz, 1H), 4.07 (tt, J=1.2, 5.6 Hz, 2H)

Step 2: A mixture of N-allyl-4-bromo-2-iodo-benzamide (5.00 g, 13.7 mmol, 1.00 eq.), triethylamine (3.46 g, 34.2 mmol, 4.75 mL, 2.50 eq.), palladium acetate (307 mg, 1.37 mmol, 0.10 eq.) and tris-o-tolylphosphine (1.37 g, 4.51 mmol, 0.33 eq) in dimethyl formamide (50 mL) was degassed and stirred at 120° C. for 3 hours under nitrogen atmosphere. The reaction mixture was diluted with water (100 mL) and extracted with ethyl acetate (50 mL×3). The combined organic layers were washed with brine (100 mL), dried over anhydrous sodium sulfate, filtered and concentrated under reduced pressure and the residue was purified by column chromatography (SiO₂, Ethyl acetate/Petroleum ether 10-100%) to give 6-bromo-4-methyl-2H-isoquinolin-1-one (1.00 g, 30.8% yield) as a yellow solid. LCMS [M+1]⁺=238.0. ¹H NMR (400 MHz, CD₃OD) δ=8.22 (d, J=8.4 Hz, 1H), 7.90 (d, J=2.0 Hz, 1H), 7.68 (dd, J=2.0, 8.4 Hz, 1H), 7.05 (d, J=1.2 Hz, 1H), 2.28 (d, J=1.2 Hz, 3H).

Step 3: To a solution of 6-bromo-4-methyl-2H-isoquinolin-1-one (500 mg, 2.10 mmol, 1.00 eq.) in THF (40.0 mL) was added sodium hydride (101 mg, 2.52 mmol, 60.0% purity, 1.20 eq.) at 0° C. and stirred at 0° C. for 0.5 hour. Then benzenesulfonyl chloride (445 mg, 2.52 mmol, 323 μL, 1.20 eq.) was added at 20° C., the mixture was warmed to 20° C. and stirred at 20° C. for 11.5 hours. The reaction mixture was diluted with water (50 mL) and extracted with ethyl acetate (30 mL×3). The combined organic layers were washed with brine (50 mL), dried over anhydrous sodium sulfate, filtered and concentrated under reduced pressure to give a residue The residue was purified by column chromatography (SiO₂, Ethyl acetate/Petroleum ether 10-100/0) to give 2-(benzenesulfonyl)-6-bromo-4-methyl-isoquinolin-1-one (713 mg, 89.8% yield) as a yellow solid. LCMS [M+1]⁺=377.9. ¹H NMR (400 MHz, DMSO-d₆) δ=8.09 (dd, J=1.2, 8.4 Hz, 2H), 8.02 (d, J=8.4 Hz, 1H), 7.87 (dd, J=1.6, 9.6 Hz, 2H), 7.78-7.65 (m, 4H), 2.31 (d, J=1.2 Hz, 3H)

Step 4: A mixture of 2-(benzenesulfonyl)-6-bromo-4-methyl-isoquinolin-1-one (250 mg, 661 μmol, 1.00 eq.), N-bromosuccinimide (165 mg, 926 μmol, 1.40 eq.) and BPO (16.0 mg, 66.1 μmol, 0.10 eq.) in carbon tetrachloride (30 mL) was degassed stirred at 80° C. for 13 hours under nitrogen atmosphere. The reaction mixture was concentrated under reduced pressure and the residue was purified by column chromatography (SiO₂, Ethyl acetate/Petroleum ether 10-35%) to give 2-(benzenesulfonyl)-6-bromo-4-(bromomethyl)isoquinolin-1-one (186 mg, 61.6% yield) as a white solid. LCMS [M+1]⁺=457.9.

Step 5: To a solution of 2-(benzenesulfonyl)-6-bromo-4-(bromomethyl)isoquinolin-1-one (120 mg, 263 μmol, 1.00 eq.) in dioxane (2.0 mL) was added diisopropylethylamine (170 mg, 1.31 mmol, 229 μL, 5.00 eq.) and (2,4-dimethoxyphenyl)methanamine (52.7 mg, 315 μmol, 47.5 μL, 1.20 eq.). The mixture was stirred at 50° C. for 2 hours. The reaction mixture was diluted with water (50 mL) and extracted with ethyl acetate (30 mL×3). The combined organic layers were washed with brine (50 mL), dried over anhydrous sodium sulfate, filtered and concentrated under reduced pressure and the residue was purified by prep-TLC (SiO₂, Petroleum ether/Ethyl acetate 1:1) to give 2-(benzenesulfonyl)-6-bromo-4-[[(2,4-dimethoxyphenyl)methylamino]methyl]isoquinolin-1-one (90.0 mg, 63.1% yield) as a yellow solid. LCMS [M+1]⁺=545.0. ¹H NMR (400 MHz, CDCl₃) δ=8.15 (s, 1H), 8.14-8.10 (m, 2H), 7.97-7.95 (m, 1H), 7.94 (d, J=1.2 Hz, 2H), 7.87 (d, J=1.6 Hz, 1H), 7.70-7.65 (m, 1H), 7.60-7.58 (m, 1H), 7.17 (d, J=8.0 Hz, 1H), 6.50 (d, J=2.4 Hz, 1H), 6.47 (dd, J=2.4, 8.0 Hz, 1H), 3.85 (s, 6H), 3.84-3.81 (m, 4H).

Step 6: To a solution of 2-(benzenesulfonyl)-6-bromo-4-[[(2,4-dimethoxyphenyl)methylamino]methyl]isoquinolin-1-one (130 mg, 239 μmol, 1.00 eq.) in water (5.0 mL) and methyl alcohol (5.0 mL) was added sodium hydroxide (191 mg, 4.78 mmol, 20.0 eq.). The mixture was stirred at 70° C. for 12 hours. The reaction mixture was concentrated under reduced pressure and the residue was purified by column chromatography (SiO₂, Ethyl acetate/Petroleum ether 50-100%, then Dichloromethane/Methyl alcohol=10:1) to give 6-bromo-4-[[(2,4-dimethoxyphenyl)methylamino]methyl]-2H-isoquinolin-1-one (78.0 mg, crude) a yellow solid which used into the next step without additional purification. LCMS [M+1]⁺=405.1. ¹H NMR (400 MHz, CDCl₃) δ=7.91 (br d, J=4.0 Hz, 1H), 7.59 (br s, 1H), 7.48 (br d, J=8.4 Hz, 1H), 7.32 (s, 1H), 7.23 (d, J=8.0 Hz, 1H), 6.56 (d, J=2.0 Hz, 1H), 6.51 (dd, J=2.0, 8.0 Hz, 1H), 3.90 (s, 3H), 3.86-3.80 (m, 6H)

Step 7: To a solution of 6-bromo-4-[[(2,4-dimethoxyphenyl)methylamino]methyl]-2H-isoquinolin-1-one (50.0 mg, 124 μmol, 1.00 eq.), Intermediate DS (120 mg, 124 μmol, 37.1% purity, 1.00 eq.) in dioxane (2.0 mL) and water (0.40 mL) was added sodium bicarbonate (20.8 mg, 248 μmol, 9.64 μL, 2.00 eq.) and Pd(dtbpf)Cl₂ (8.08 mg, 12.4 μmol, 0.10 eq.). The mixture was stirred at 80° C. for 12 hours. The reaction mixture was concentrated under reduced pressure and the residue was purified by prep-TLC (SiO₂, Dichloromethane/Methyl alcohol 10:1) to give 2-[4-[4-[[(2,4-dimethoxyphenyl)methylamino]methyl]-1-oxo-2H-isoquinolin-6-yl]-2-methyl-pyrazol-3-yl]naphthalene-1-carbonitrile (30.0 mg, 25.8% yield) as a yellow solid. LCMS [M+1]⁺=556.3

Step 8: 2-[4-[4-[[(2,4-dimethoxyphenyl)methylamino]methyl]-1-oxo-2H-isoquinolin-6-yl]-2-methyl-pyrazol-3-yl]naphthalene-1-carbonitrile (10.0 mg, 10.7 μmol, 59.3% purity, 1.00 eq.) in trifluoroacetic acid (1.54 g, 13.5 mmol, 1.0 mL, 1265 eq.) were taken up into a microwave tube. The sealed tube was heated at 100° C. for 2 hours under microwave. The reaction mixture was concentrated under reduced pressure and the residue was purified by prep-HPLC (column: 3_Phenomenex Luna C18 75×30 mm×3 μm; mobile phase: [water (0.05% HCl)-ACN]; B %: 15%-35%, 7 min) to give 2-[4-[4-(aminomethyl)-1-oxo-2H-isoquinolin-6-yl]-2-methyl-pyrazol-3-yl]naphthalene-1-carbonitrile (2.98 mg, 62.4% yield) as a yellow solid. LCMS [M−16]⁺=389.2. ¹H NMR (400 MHz, DMSO-d₆) δ=11.41 (br d, J=6.0 Hz, 1H), 8.51 (d, J=8.4 Hz, 1H), 8.35 (s, 1H), 8.32-8.11 (m, 5H), 7.91 (s, 1H), 7.90-7.87 (m, 1H), 7.85 (d, J=7.6 Hz, 1H), 7.82 (d, J=1.2 Hz, 1H), 7.78 (d, J=8.4 Hz, 1H), 7.35 (d, J=6.0 Hz, 1H), 6.94 (dd, J=1.2, 8.4 Hz, 1H), 4.10-3.95 (m, 2H), 3.76 (s, 3H)

### Example 19-1

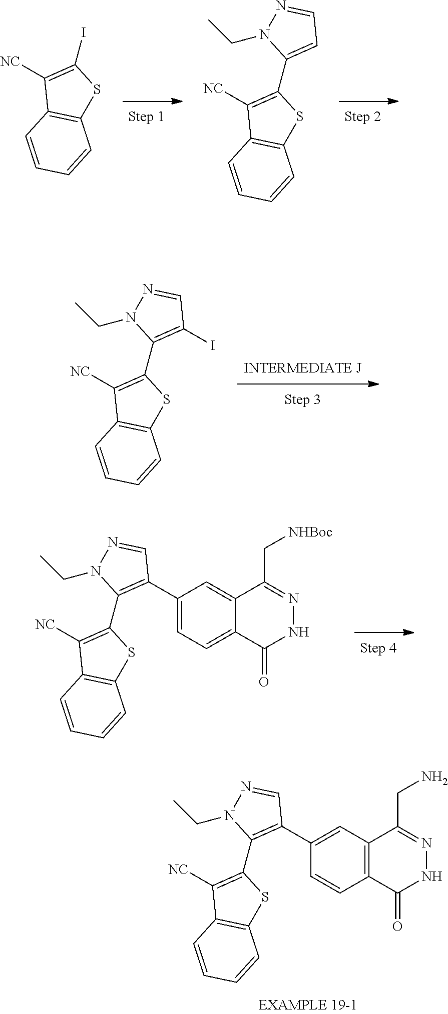 [SMILES: N#Cc1c(I)sc2ccccc12 ; CCn1nccc1-c1sc2ccccc2c1C#N ; CCn1ncc(I)c1-c1sc2ccccc2c1C#N ; CCn1ncc(-c2ccc3c(=O)[nH]nc(CNC(=O)OC(C)(C)C)c3c2)c1-c1sc2ccccc2c1C#N ; CCn1ncc(-c2ccc3c(=O)[nH]nc(CN)c3c2)c1-c1sc2ccccc2c1C#N]

Step 1: To a 100 mL three-necked flask was added 2-iodobenzothiophene-3-carbonitrile (500 mg, 1.75 mmol, 1.00 eq.), 1-ethyl-5-(4,4,5,5-tetramethyl-1,3,2-dioxaborolan-2-yl)pyrazole (389.49 mg, 1.75 mmol, 1.00 eq.), Pd(dppf)Cl₂ (128 mg, 175 μmol, 0.10 eq.), sodium carbonate (371.76 mg, 3.51 mmol, 2.00 eq.), dioxane (10.0 mL) and water (3.0 mL). The reaction mixture was degassed and heated to 90° C. for 14 hours. The reaction was poured into water (30 mL) and then extracted with ethyl acetate (2×20 mL). The combined organic layers were dried over sodium sulfate and then concentrated under reduced pressure to get the residue. The residue was purified by silica gel column chromatography (ethyl acetate/petroleum ether 0-10%) to give 2-(1-ethyl-1H-pyrazol-5-yl)benzo[b]thiophene-3-carbonitrile (378 mg, 85.1% yield) as a white solid. ¹H NMR (400 MHz, CDCl₃-d) δ=8.03 (d, J=8.0 Hz, 1H), 7.91 (d, J=8.0 Hz, 1H), 7.66 (d, J=2.0 Hz, 1H), 7.63-7.57 (m, 1H), 7.57-7.51 (m, 1H), 6.75 (d, J=2.0 Hz, 1H), 4.38 (q, J=7.2 Hz, 2H), 1.51 (t, J=7.3 Hz, 3H).

Step 2: To a solution of 2-(1-ethyl-1H-pyrazol-5-yl)benzo[b]thiophene-3-carbonitrile (360 mg, 1.42 mmol, 1.00 eq.) in acetonitrile (5.0 mL) was added NIS (1.60 g, 7.11 mmol, 5.00 eq.) and acetic acid (171 mg, 2.84 mmol, 163 μL, 2.00 eq.). The reaction mixture was stirred at 90° C. for 1 hour. The reaction was quenched with saturated aqueous sodium bicarbonate (40.0 mL) and the resulting mixture was extracted with ethyl acetate (2×20 mL). The combined organic extracts were dried anhydrous sodium sulfate, filtered and concentrated under reduced pressure to afford 2-(1-ethyl-4-iodo-1H-pyrazol-5-yl)benzo[b]thiophene-3-carbonitrile (500 mg, 92.8% yield) as a white solid. ¹H NMR (400 MHz, CDCl₃-d) δ=8.08 (d, J=7.9 Hz, 1H), 7.96 (d, J=7.6 Hz, 1H), 7.71 (s, 1H), 7.66-7.56 (m, 2H), 4.24 (q, J=7.2 Hz, 2H), 1.44 (t, J=7.2 Hz, 3H).

Step 3: A mixture of 2-(1-ethyl-4-iodo-1H-pyrazol-5-yl)benzo[b]thiophene-3-carbonitrile (100 mg, 264 μmol, 1.00 eq.), Intermediate J (116 mg, 290 μmol, 1.10 eq.), Pd(dppf)Cl₂ (19.30 mg, 26.37 μmol, 0.10 eq.) and sodium carbonate (83.9 mg, 791.1 μmol, 3.00 eq.) in dioxane (3.0 mL) and water (1.0 mL) was degassed and heated to 90° C. for 3 hours. The reaction was quenched with saturated aqueous ammonium chloride (40.0 mL) and the resulting mixture was extracted with ethyl acetate (2×20 mL). The combined organic extracts were dried over anhydrous sodium sulfate, filtered and concentrated under reduced pressure to give a crude product. The residue was purified by silica gel column chromatography (Ethyl acetate/Petroleum ether 10-35%) to get tert-butyl N-[[7-[5-(3-cyanobenzothiophen-2-yl)-1-ethyl-pyrazol-4-yl]-4-oxo-3H-phthalazin-1-yl]methyl]carbamate (100 mg, 72.0% yield) as a brown gum. LCMS [M+1]⁺=527.2

Step 4: To a solution of tert-butyl N-[[7-[5-(3-cyanobenzothiophen-2-yl)-1-ethyl-pyrazol-4-yl]-4-oxo-3H-phthalazin-1-yl]methyl]carbamate (100 mg, 190 μmol, 1.00 eq.) in dichloromethane (2.0 mL) was added trifluoroacetic acid (770 mg, 6.75 mmol, 500 μL, 35.6 eq.). The reaction mixture was stirred at 15° C. for 1 hour. The reaction mixture was concentrated under reduced pressure and the residue was purified by prep-HPLC (column: Welch Xtimate C18 150×25 mm×5 μm; mobile phase: [water (0.05% HCl)-ACN]; B %: 8/0-38%, 10 min) to afford 2-[4-[4-(aminomethyl)-1-oxo-2H-phthalazin-6-yl]-2-ethyl-pyrazol-3-yl]benzothiophene-3-carbonitrile (34.2 mg, 38.5% yield) as a white solid. LCMS [M+1]⁺=426.9. ¹H NMR (400 MHz, MeOD-d₄) δ=12.88 (s, 1H), 8.46 (br s, 3H), 8.39 (s, 1H), 8.29 (d, J=6.8 Hz, 1H), 8.10 (d, J=8.4 Hz, 1H), 8.04-7.97 (m, 2H), 7.73-7.65 (m, 2H), 7.47 (dd, J=1.6, 8.4 Hz, 1H), 4.38 (br s, 2H), 4.22 (q, J=7.2 Hz, 2H), 1.38 (t, J=7.2 Hz, 3H).

### Example 19-2

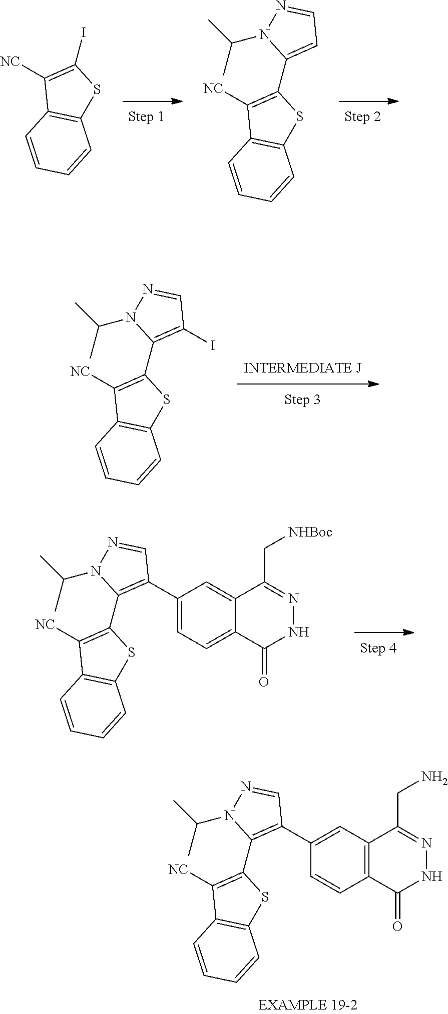 [SMILES: N#Cc1c(I)sc2ccccc12 ; CC(C)n1nccc1-c1sc2ccccc2c1C#N ; CC(C)n1ncc(I)c1-c1sc2ccccc2c1C#N ; CC(C)n1ncc(-c2ccc3c(=O)[nH]nc(CNC(=O)OC(C)(C)C)c3c2)c1-c1sc2ccccc2c1C#N ; CC(C)n1ncc(-c2ccc3c(=O)[nH]nc(CN)c3c2)c1-c1sc2ccccc2c1C#N]

Example 19-2 was prepared following the same reaction sequence as Example 19-1 except for using 1-isopropyl-5-(4,4,5,5-tetramethyl-1,3,2-dioxaborolan-2-yl)pyrazole in Step 1 to afford 2-(4-(4-(aminomethyl)-1-oxo-1,2-dihydrophthalazin-6-yl)-1-isopropyl-1H-pyrazol-5-yl)benzo[b]thiophene-3-carbonitrile, Example 19-2 (70.1 mg, 158 μmol) as a yellow solid. LCMS [M+1]⁺: 441.2. ¹H NMR (400 MHz, MeOD): δ=8.24-8.21 (m, 2H), 8.16-8.11 (m, 1H), 8.02-7.97 (m, 1H), 7.84 (d, J=1.2 Hz, 1H), 7.68-7.62 (m, 3H), 4.63-4.56 (m, 1H), 4.38 (s, 2H), 1.55 (d, J=6.4 Hz, 6H).

### Example 19-3

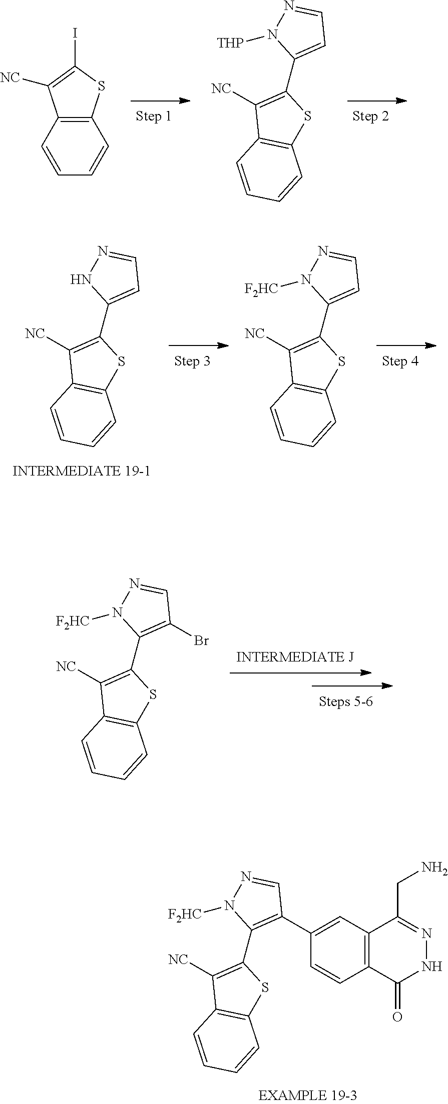

Step 1: To a solution of 1-tetrahydropyran-2-yl-5-(4,4,5,5-tetramethyl-1,3,2-dioxaborolan-2-yl)pyrazole (1.46 g, 5.26 mmol, 1.50 eq.) and 2-iodobenzothiophene-3-carbonitrile (1.00 g, 3.51 mmol, 1.00 eq.) in 1,4-dioxane (15 mL) and water (3.0 mL) was added sodium carbonate (744 mg, 7.01 mmol, 2.00 eq.) and Pd(dppf)Cl₂ (257 mg, 351 μmol, 0.10 eq.), the mixture was degassed and stirred at 80° C. under nitrogen atmosphere for 16 hours. The mixture was diluted with water (60 mL) and extracted with ethyl acetate (80 mL×3). The combined organic extracts were washed brine (30 mL×3) and then dried over anhydrous sodium sulfate, filtered and concentrated in vacuum and the residue was purified by silica gel column chromatography (ethyl acetate/petroleum ether 5-15%) to give 2-(1-(tetrahydro-2H-pyran-2-yl)-1H-pyrazol-5-yl)benzo[b]thiophene-3-carbonitrile (840 mg, 77.4% yield) as a yellow solid. LCMS [M+1]⁺: 225.9. ¹H NMR (400 MHz, CDCl₃) δ=8.03 (d, J=8.0 Hz, 1H), 7.92 (d, J=8.0 Hz, 1H), 7.72 (d, J=2.0 Hz, 1H), 7.60-7.57 (m, 1H), 7.56-7.51 (m, 1H), 6.87 (d, J=2.0 Hz, 1H), 5.49 (dd, J=2.4, 9.6 Hz, 1H), 4.13-4.05 (m, 2H), 2.21-2.09 (m, 4H), 1.83-1.72 (m, 2H).

Step 2: To a solution of 2-(1-(tetrahydro-2H-pyran-2-yl)-1H-pyrazol-5-yl)benzo[b]thiophene-3-carbonitrile (800 mg, 2.59 mmol, 1.00 eq.) in methanol (12.0 mL) was added concentrated hydrochloric acid (12.0 M, 21.6 μL, 0.10 eq.) at 20° C. and the resulting was stirred for 1 hour. The reaction was quenched with saturated sodium bicarbonate (10.0 mL). The mixture was extracted with ethyl acetate (20 mL×3) and brine (20 mL) and then dried over sodium sulfate, filtered and concentrated in vacuum and the crude product was used directly without further purification to give 2-(1H-pyrazol-5-yl)benzo[b]thiophene-3-carbonitrile, Intermediate 19-1 (800 mg, crude) as yellow solid. LCMS [M+1]⁺: 226.2. ¹H NMR (400 MHz, CDCl₃-d) δ=7.97 (d, J=8.0 Hz, 1H), 7.87 (d, J=8.0 Hz, 1H), 7.77 (d, J=2.4 Hz, 1H), 7.56-7.51 (m, 1H), 7.49-7.44 (m, 1H), 7.24 (d, J=2.5 Hz, 1H).

Step 3: 2-(1H-pyrazol-5-yl)benzo[b]thiophene-3-carbonitrile (100 mg, 444 μmol, 1.00 eq.) and potassium fluoride (129 mg, 2.22 mmol, 52.0 μL, 5.00 eq.) in acetonitrile (2.0 mL) was added 1-[[bromo(difluoro)methyl]-ethoxy-phosphoryl]oxyethane (237 mg, 889 μmol, 2.00 eq.) at 20° C., the mixture was stirred at 30° C. for 30 hours. The reaction mixture was concentrated under reduced pressure and the residue was purified by prep-TLC (silicon dioxide, petroleum ether/ethyl acetate 5:1) to give compound 2-(1-(difluoromethyl)-1H-pyrazol-5-yl)benzo[b]thiophene-3-carbonitrile (13.0 mg, 10.6% yield) as a yellow solid. LCMS [M+1]⁺]: 276.0. ¹H NMR (400 MHz, CDCl₃) δ=8.05 (dd, J=0.8, 7.2 Hz, 1H), 7.93 (d, J=7.6 Hz, 1H), 7.82 (d, J=2.0 Hz, 1H), 7.65-7.55 (m, 2H), 7.38 ((t, J=58.4 Hz, 1H), 6.93 (d, J=2.0 Hz, 1H).

Step 4: To a solution of 2-(1-(difluoromethyl)-1H-pyrazol-5-yl)benzo[b]thiophene-3-carbonitrile (30.0 mg, 109 μmol, 1.00 eq.) in acetic acid (1.0 mL) was added N-bromosuccinimide (194 mg, 1.09 mmol, 10.0 eq.) at 20° C., the mixture was stirred at 80° C. for 16 hours. The mixture was diluted with water (10.0 mL) and extracted with ethyl acetate (10.0 mL×3). The combined organic extracts were washed brine (10.0 mL×3) and then dried over anhydrous sodium sulfate, filtered and concentrated in vacuum and the residue was purified by prep-TLC (silicon dioxide, petroleum ether/ethyl acetate 5:1) to give 2-(4-bromo-1-(difluoromethyl)-1H-pyrazol-5-yl)benzo[b]thiophene-3-carbonitrile (13.0 mg, 33.7% yield) as a yellow solid. LCMS [M+1]⁺: 355.9

Step 5: To a mixture of 2-(4-bromo-1-(difluoromethyl)-1H-pyrazol-5-yl)benzo[b]thiophene-3-carbonitrile (13.0 mg, 36.7 μmol, 1.00 eq), Intermediate J (17.7 mg, 44.1 μmol, 1.20 eq.) in dioxane (1.0 mL) and water (0.20 mL) were added (Ad₂ n-BuP)—Pd G3 (2.67 mg, 3.67 μmol, 0.10 eq.) and sodium bicarbonate (9.25 mg, 110 μmol, 4.28 μL, 3.00 eq.). The reaction mixture was degassed and heated at 80° C. for 6 hours. Water (25 mL) was added and the mixture was extracted with ethyl acetate (30 mL). The organic layer was dried over sodium sulfate and then concentrated under vacuum to get the residue. The residue was purified by prep-TLC (dichloromethane/methanol 20:1) to afford tert-butyl ((7-(5-(3-cyanobenzo[b]thiophen-2-yl)-1-(difluoromethyl)-1H-pyrazol-4-yl)-4-oxo-3,4-dihydrophthalazin-1-yl)methyl)carbamate (15.0 mg, 74.5% yield) as a white solid. LCMS [M+1]⁺: 549.2.

Step 6: To a solution of tert-butyl ((7-(5-(3-cyanobenzo[b]thiophen-2-yl)-1-(difluoromethyl)-1H-pyrazol-4-yl)-4-oxo-3,4-dihydrophthalazin-1-yl)methyl)carbamate (10.0 mg, 18.2 μmol, 1.00 eq.) in dichloromethane (1.0 mL) was added trifluoroacetic acid (2.08 mg, 18.2 μmol, 1.35 μL, 1.00 eq.) and the resulting mixture was stirred at 20° C. for 1 hour. The reaction mixture was concentrated under reduced pressure and the residue was purified by prep-HPLC (column: Phenomenex Luna C18 150×25 mm×10 μm; mobile phase: [water (0.05% HCl)-ACN]; B %: 14%-44%, 10 min) to give 2-(4-(4-(aminomethyl)-1-oxo-1,2-dihydrophthalazin-6-yl)-1-(difluoromethyl)-1H-pyrazol-5-yl)benzo[b]thiophene-3-carbonitrile (3.79 mg, 40.9% yield) as a white solid. LCMS [M+1]⁺: 449.1. ¹H NMR (400 MHz, CD₃OD-d₄) δ=8.41 (s, 1H), 8.27 (d, J=8.4 Hz, 1H), 8.20-8.10 (m, 1H), 8.02-7.95 (m, 1H), 7.93 (d, J=1.2 Hz, 1H), 7.84-7.54 (m, 4H), 4.41 (s, 2H).

### Example 19-4

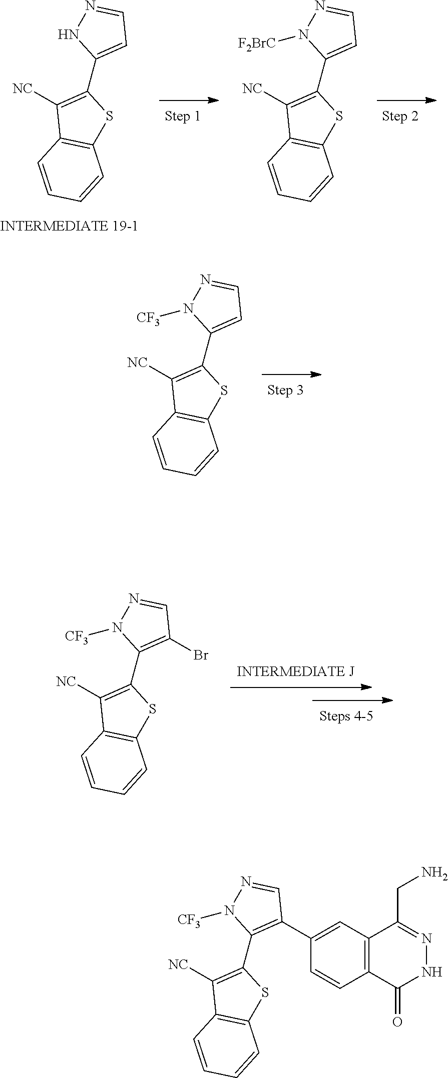

Step 1: To a solution of Intermediate 19-1 (800 mg, 3.55 mmol, 1.00 eq.) in DMF (8.00 mL) was added sodium hydride (185 mg, 4.62 mmol, 60.0% purity, 1.30 eq.) under nitrogen at 0° C. The mixture was stirred at 15° C. for 30 min. Then difluorodibromomethane (1.49 g, 5.33 mmol, 1.50 eq.) in dimethylformamide (2.0 mL) was added dropwise. The mixture was stirred at 15° C. for 15 hours. Water (50 mL) was added and the mixture was extracted with ethyl acetate (20 mL×3). The organic layer was dried over sodium sulfate and then concentrated under vacuum to produce a residue. The residue was purified by silica gel column chromatography (ethyl acetate/petroleum ether 5-30%) to give 2-(1-(bromodifluoromethyl)-1H-pyrazol-5-yl)benzo[b]thiophene-3-carbonitrile (240 mg, 19.1% yield) as a yellow solid. LCMS [M+3]⁺: 355.9. ¹H NMR (400 MHz, CDCl₃) δ=8.04 (d, J=7.6 Hz, 1H), 7.95-7.90 (m, 2H), 7.64-7.55 (m, 2H), 6.81 (s, 1H).

Step 2: To a solution of 2-(1-(bromodifluoromethyl)-1H-pyrazol-5-yl)benzo[b]thiophene-3-carbonitrile (240 mg, 678 μmol, 1.00 eq.) in dichloromethane (5.0 mL) was added silver fluoroborate (264 mg, 1.36 mmol, 2.00 eq.) under nitrogen at −70° C. The mixture was stirred at 15° C. for 3 hours. After that time water (20 mL) was added and the mixture was extracted with dichloromethane (10.0 mL 3). The organic layer was dried over sodium sulfate and then concentrated under vacuum to produce a residue. The residue was purified by silica gel column chromatography (ethyl acetate/petroleum ether 20-100%) to give 2-(1-(trifluoromethyl)-1H-pyrazol-5-yl)benzo[b]thiophene-3-carbonitrile (173 mg, 87.1% yield) as a yellow solid. LCMS [M+1]⁺: 294.0. ¹H NMR (400 MHz, CDCl₃) δ=8.04 (d, J=7.6 Hz, 1H), 7.95-7.86 (m, 2H), 7.65-7.55 (m, 2H), 6.83 (s, 1H).

Step 3: To a solution of 2-(1-(trifluoromethyl)-1H-pyrazol-5-yl)benzo[b]thiophene-3-carbonitrile (170 mg, 580 μmol, 1.00 eq.) in acetic acid (2.0 mL) was added N-bromosuccinimide (2.06 g, 11.59 mmol, 20.0 eq.) at 15° C. The mixture was stirred at 120° C. for 16 hours. Water (20 mL) was added and the mixture was extracted with ethyl acetate (20 mL×3). The combined organic layers were washed brine (10.0 mL×3) and then dried over anhydrous sodium sulfate, filtered and concentrated in vacuum and the residue was purified by prep-TLC (silicon dioxide, petroleum ether/ethyl acetate 3:1) to give 2-(4-bromo-1-(trifluoromethyl)-1H-pyrazol-5-yl)benzo[b]thiophene-3-carbonitrile (28.0 mg, 13.0% yield) as a white solid. LCMS [M+1]⁺: 372.1. ¹H NMR (400 MHz, CDCl₃) δ=8.14-8.04 (m, 1H), 8.03-7.93 (m, 1H), 7.90 (s, 1H), 7.69-7.57 (m, 2H).

Step 4: To a solution of 2-(4-bromo-1-(trifluoromethyl)-1H-pyrazol-5-yl)benzo[b]thiophene-3-carbonitrile (28.0 mg, 75.2 μmol, 1.00 eq.) and Intermediate J (36.2 mg, 90.3 μmol, 1.20 eq.) in dioxane (1.0 mL) and water (0.20 mL) was added sodium bicarbonate (19.0 mg, 226 μmol, 3.00 eq.) and (Ad₂n-BuP)—Pd G3 (5.48 mg, 7.52 μmol, 0.10 eq.) at 15° C. The reaction mixture was degassed and then heated at 80° C. for 16 hours. Water (10.0 mL) was added and the mixture was extracted with ethyl acetate (10.0 mL×3). The combined organic extracts were washed brine (10.0 mL), dried over anhydrous sodium sulfate, filtered and concentrated in vacuum and the residue was purified by prep-(silicon dioxide, petroleum ether/ethyl acetate 1:1) to give tert-butyl ((7-(5-(3-cyanobenzo[b]thiophen-2-yl)-1-(trifluoromethyl)-1H-pyrazol-4-yl)-4-oxo-3,4-dihydrophthalazin-1-yl)methyl)carbamate (20.0 mg, 46.9% yield) as a white solid. LCMS [M+1]⁺: 567.2.

Step 5: To a solution of tert-butyl ((7-(5-(3-cyanobenzo[b]thiophen-2-yl)-1-(trifluoromethyl)-1H-pyrazol-4-yl)-4-oxo-3,4-dihydrophthalazin-1-yl)methyl)carbamate (20.0 mg, 35.3 μmol, 1.00 eq.) in dichloromethane (1.0 mL) was added trifluoroacetic acid (308 mg, 2.70 mmol, 0.20 mL, 76.5 eq) and the mixture was stirred at 20° C. for 1 hour. The reaction mixture was concentrated under reduced pressure and the residue was purified by prep-HPLC (column: Welch Xtimate C18 150×25 mm×5 μm; mobile phase: [water (0.05% HCl)-ACN]; B %: 19%-49%, 10 min) to give 2-(4-(4-(aminomethyl)-1-oxo-1,2-dihydrophthalazin-6-yl)-1-(trifluoromethyl)-1H-pyrazol-5-yl)benzo[b]thiophene-3-carbonitrile (13.7 mg, 75.2% yield) as a white solid. LCMS [M+1]⁺: 467.0. ¹H NMR (400 MHz, CD₃OD) δ=8.52 (s, 1H), 8.27 (d, J=8.4 Hz, 1H), 8.19-8.10 (m, 1H), 8.04-7.96 (m, 1H), 7.94 (d, J=1.2 Hz, 1H), 7.76-7.62 (m, 3H), 4.42-4.39 (m, 2H).

### Examples 20-1 and 20-2

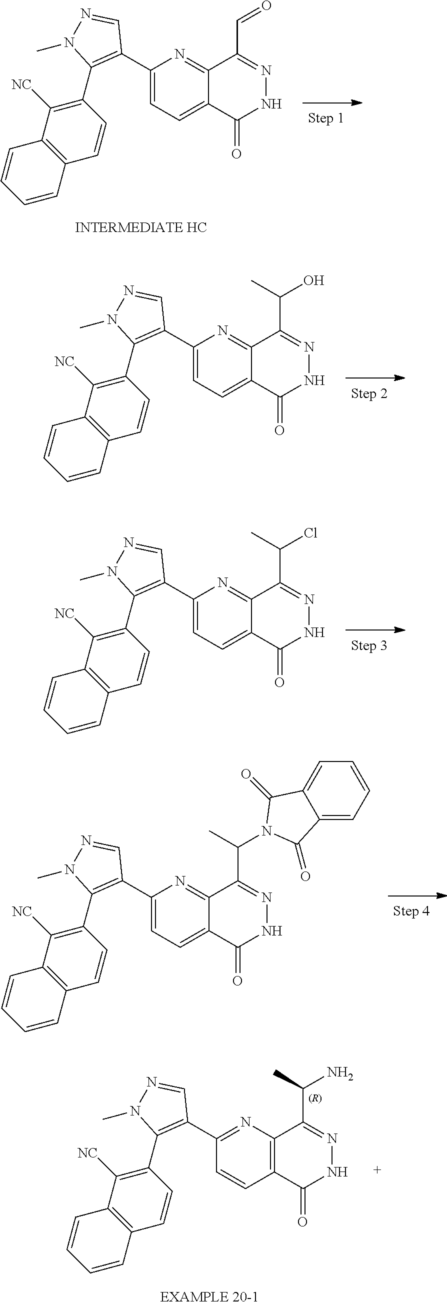

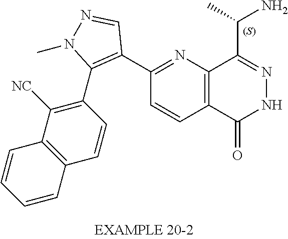

Step 1: To a solution of 2-[5-(1-isocyano-2-naphthyl)-1-methyl-pyrazol-4-yl]-5-oxo-6H-pyrido[2,3-d]pyridazine-8-carbaldehyde (240 mg, 591 μmol, 1.00 eq.) in THF (5 mL) was added dropwise methylmagnesium bromide (3.0 M, 1.97 mL, 10.0 eq.) at −78° C. The resulting mixture was stirred at 15° C. for 2 hours. The reaction was quenched with saturated ammonium chloride (20 mL). The mixture was extracted with DCM (3×30 mL). The combined organic extracts were washed with saturated ammonium chloride (20 mL) and brine (20 mL) and then dried over sodium sulfate. The crude product was triturated with DCM (5 mL) at 15° C. for 15 min to give 8-(1-hydroxyethyl)-2-[5-(1-isocyano-2-naphthyl)-1-methyl-pyrazol-4-yl]-6H-pyrido[2,3-d]pyridazin-5-one (160 mg, 352 μmol, 60% yield) as a yellow solid. 1H NMR (400 MHz, DMSO-d₆) δ=12.61 (s, 1H), 8.55-8.44 (m, 3H), 8.27 (t, J=9.2 Hz, 1H), 8.20 (d, J=8.4 Hz, 1H), 8.15 (t, J=8.8 Hz, 1H), 7.93-7.70 (m, 3H), 4.24-3.80 (m, 2H), 3.77-3.68 (m, 3H), 3.30 (s, 3H), 0.52-0.27 (m, 3H).

Step 2: To a solution of 8-(1-hydroxyethyl)-2-[5-(1-isocyano-2-naphthyl)-1-methyl-pyrazol-4-yl]-6H-pyrido[2,3-d]pyridazin-5-one (160 mg, 379 μmol, 1.00 eq.) in thionyl chloride (52.5 g, 441 mmol, 32.0 mL, 116 eq.) was added DMF (2.8 mg, 37.9 μmol, 2.9 uL, 0.1 eq.). The mixture was stirred at 15° C. for 20 minutes. The reaction mixture was diluted with DCM (20 mL) and slowly added to a saturated aqueous sodium bicarbonate (20 mL). The mixture was extracted with DCM (3×20 mL). The organic layer was dried over anhydrous sodium sulfate, filtered and concentrated under vacuum to give 8-(1-chloroethyl)-2-[5-(1-isocyano-2-naphthyl)-1-methyl-pyrazol-4-yl]-6H-pyrido[2,3-d]pyridazin-5-one (150 mg, 340 μmol, 90% yield) as a yellow solid. ¹H NMR (400 MHz, DMSO-d₆) δ=12.83-12.80 (d, J=9.6 Hz, 1H), 8.57 (d, J=1.6 Hz, 1H), 8.55-8.45 (m, 2H), 8.35-8.22 (m, 3H), 8.15 (d, J=8.4 Hz, 1H), 7.97-7.82 (m, 3H), 7.66 (d, J=8.4 Hz, 1H), 4.12 (d, J=6.8 Hz, 0.5 H), 4.05 (d, J=6.8 Hz, 0.5 H), 3.76 (d, J=8.8 Hz, 3H), 1.00 (d, J=6.8 Hz, 1.5H), 0.87 (d, J=6.8 Hz, 1.5H).

Step 3: To a solution of 8-(1-chloroethyl)-2-[5-(1-isocyano-2-naphthyl)-1-methyl-pyrazol-4-yl]-6H-pyrido [2, 3-d] pyridazin-5-one (140 mg, 318 μmol, 1.00 eq.) in DMF (3 mL) was added (1, 3-dioxoisoindolin-2-yl)potassium (65 mg, 351 μmol, 1.11 eq.). The mixture was stirred at 70° C. for 0.5 hours. After being cooled to 20° C., the mixture was concentrated under vacuum. The residue was purified by column chromatography on silica gel (DCM:methanol=20/1) to give 2-[1-[2-[5-(1-isocyano-2-naphthyl)-1-methyl-pyrazol-4-yl]-5-oxo-6H-pyrido[2,3-d]pyridazin-8-yl]ethyl] isoindoline-1,3-dione (65 mg, 110 dichloromethane mol, 35% yield) as a yellow solid. ¹H NMR (400 MHz, DMSO-d₆) δ=12.60-12.56 (m, 1H), 8.52-8.41 (m, 3H), 8.24-8.16 (m, 2H), 8.12 (d, J=8.4 Hz, 1H), 8.04 (d, J=8.4 Hz, 1H), 7.96-7.92 (m, 1H), 7.89-7.77 (m, 6H), 7.76-7.64 (m, 2H), 5.02 (s, 1H), 4.75 (d, J=7.2 Hz, 1H), 3.75 (d, J=13.6 Hz, 3H), 1.08 (d, J=7.2 Hz, 1H), 1.02 (d, J=7.2 Hz, 2H).

Step 4: To a solution of 2-[1-[2-[5-(1-isocyano-2-naphthyl)-1-methyl-pyrazol-4-yl]-5-oxo-6H-pyrido[2,3-d]pyridazin-8-yl]ethyl]isoindoline-1,3-dione (65 mg, 118 μmol, 1.00 eq.) in ethanol (5 mL) was added hydrazine hydrate (118 mg, 2.31 mmol, 115 uL, 19.6 eq.). The mixture was stirred at 50° C. for 24 hours. TLC (DCM:methanol 10/1) indicated 50% of starting material remained and one new spot formed. The mixture was added hydrazine hydrate (118 mg, 2.31 mmol, 115 uL, 98% purity, 19.6 eq.) and stirred at 50° C. for 2 hours. After being cooled to room temperature, the mixture was concentrated under vacuum. The residue was purified by prep-HPLC (column: Welch Xtimate C18 150×25 mm×5 μm; mobile phase: [water (0.05% HCl)-ACN]; B %: 10%-40/a, 10 min). The desired fraction was collected and lyophilized to give 8-(1-aminoethyl)-2-[5-(1-isocyano-2-naphthyl)-1-methyl-pyrazol-4-yl]-6H-pyrido[2, 3-d]pyridazin-5-one (30 mg, 64 μmol, 54% yield) as a white solid. ¹H NMR (400 MHz, DMSO-d₆) δ=12.52 (br s, 1H), 8.58-8.44 (m, 3H), 8.28 (t, J=7.6 Hz, 1H), 8.20 (br d, J=8.4 Hz, 1H), 8.16 (t, J=7.6 Hz, 1H), 7.93-7.71 (m, 3H), 3.74 (d, J=2.4 Hz, 3H), 3.20-3.07 (m, 1H), 0.46-0.31 (m, 3H). Separation of stereoisomers: 8-(1-aminoethyl)-2-[5-(1-isocyano-2-naphthyl)-1-methyl-pyrazol-4-yl]-6H-pyrido [2,3-d]pyridazin-5-one (30 mg, 64 μmol) was purified by prep-SFC column: DAICEL CHIRALPAK IG (250 mm×30 mm, 10 μm); mobile phase: [Heptane-EtOH (0.1% NH₃H₂O)]; B %: 30%-30%, 11; 160 min to give the crude products (P1 and P3). The residue (P1) was purified by prep-HPLC column: Welch Xtimate C18 150×25 mm×5 μm; mobile phase: [water (HCl)-ACN]; B %: 12/6-42%, 10 min to give EXAMPLE 20-1 8-[(1R)-1-aminoethyl]-2-[5-(1-isocyano-2-naphthyl)-1-methyl-pyrazol-4-yl]-6H-pyrido [2, 3-d]pyridazin-5-one (7.5 mg, 17 μmol, 21% yield) as a yellow solid. LCMS: [M+H] 422.0; ¹H NMR (400 MHz, MeOD-d₄) δ=8.62-8.56 (m, 1H), 8.50-8.40 (m, 2H), 8.32-8.20 (m, 2H), 8.09 (dd, J=8.4, 13.6 Hz, 1H), 7.94-7.83 (m, 2H), 7.77 (dd, J=8.4, 17.0 Hz, 1H), 4.05-4.00 (m, 1H), 3.79 (s, 3H), 0.92-0.68 (m, 3H). The residue (P3) was purified by the same prep-HPLC method to give EXAMPLE 20-2, 8-[(1S)-1-aminoethyl]-2-[5-(1-isocyano-2-naphthyl)-1-methyl-pyrazol-4-yl]-6H-pyrido [2, 3-d] pyridazin-5-one (5.3 mg, 12 μmol, 15% yield) as a yellow solid. ¹H NMR (400 MHz, MeOD-d₄) δ=8.62-8.55 (m, 1H), 8.50-8.38 (m, 2H), 8.33-8.19 (m, 2H), 8.13-8.05 (m, 1H), 7.95-7.83 (m, 2H), 7.81-7.72 (m, 1H), 4.07-3.99 (m, 1H), 3.82-3.74 (m, 3H), 0.92-0.69 (m, 3H). LCMS [M+1]⁺ 422.0.

### Example 20-3

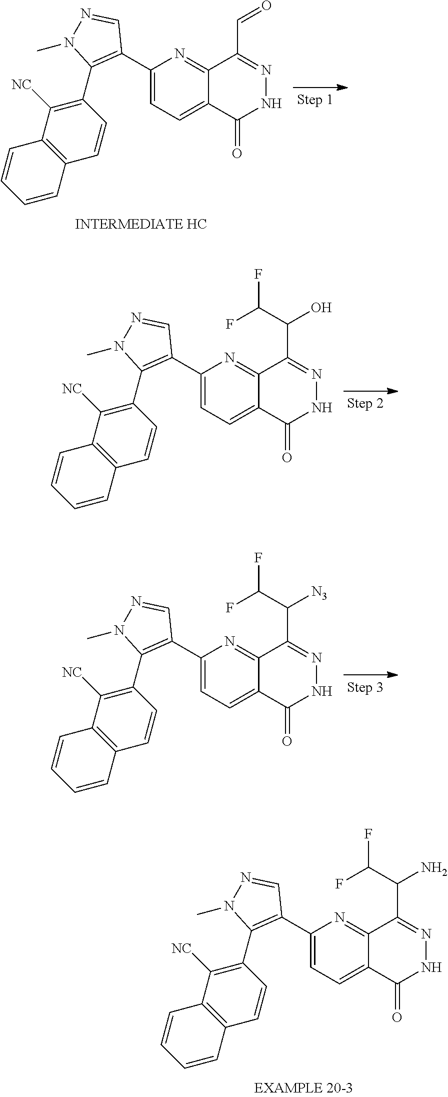 [SMILES: Cn1ncc(-c2ccc3c(=O)[nH]nc(C=O)c3n2)c1-c1ccc2ccccc2c1C#N ; Cn1ncc(-c2ccc3c(=O)[nH]nc(C(O)C(F)F)c3n2)c1-c1ccc2ccccc2c1C#N ; Cn1ncc(-c2ccc3c(=O)[nH]nc(C(N=[N+]=[N-])C(F)F)c3n2)c1-c1ccc2ccccc2c1C#N ; Cn1ncc(-c2ccc3c(=O)[nH]nc(C(N)C(F)F)c3n2)c1-c1ccc2ccccc2c1C#N]

Step 1: To a solution of 2-[5-(1-isocyano-2-naphthyl)-1-methyl-pyrazol-4-yl]-5-oxo-6H-pyrido[2,3-d]pyridazine-8-carbaldehyde (250 mg, 615 μmol, 1.00 eq.) in DMF (5 mL) was added difluoromethyl(trimethyl)silane (1.53 g, 12.3 mmol, 20.0 eq) and cesium fluoride (94 mg, 615 μmol, 1.00 eq.). The mixture was stirred at 60° C. for 12 hours. The reaction mixture was cooled to 20° C. EtOAc (40 mL) and water (40 mL) were added, and layers were separated. The aqueous phase was extracted with EtOAc (30 mL×2). Combined extracts were washed with brine (60 mL), dried over sodium sulfate, filtered and concentrated under reduced pressure and the residue was purified by column chromatography on silica gel (dichloromethane:methanol=10/1) to give 8-(2,2-difluoro-1-hydroxy-ethyl)-2-[5-(1-isocyano-2-naphthyl)-1-methyl-pyrazol-4-yl]-6H-pyrido[2,3-d]pyridazin-5-one (30 mg, 51 μmol, 8% yield) as a yellow solid. ¹H NMR (400 MHz, DMSO-d₆) δ=12.89 (s, 1H), 8.56-8.42 (m, 3H), 8.25-8.03 (m, 3H), 7.91-7.63 (m, 4H), 5.78-5.29 (m, 2H), 4.31-4.08 (m, 1H), 3.73 (s, 3H).

Step 2: To a solution of 8-(2,2-difluoro-1-hydroxy-ethyl)-2-[5-(1-isocyano-2-naphthyl)-1-methyl-pyrazol-4-yl]-6H-pyrido[2,3-d]pyridazin-5-one (40 mg, 87 μmol, 1.00 eq.) in THF (2 mL) was added [azido(phenoxy)phosphoryl]oxybenzene (120 mg, 436 μmol, 94 uL, 5 eq.) and 2,3,4,6,7,8,9,10-octahydropyrimido[1,2-a]azepine (28 mg, 184 μmol, 28 μL, 2.11 eq.) at 0° C. After the mixture was stirred at 60° C. for 12 hours, the mixture was cooled to 20° C. and concentrated under reduced pressure and the residue was purified by column chromatography on silica gel (dichloromethane/methanol=10/1) to give 8-(1-azido-2,2-difluoro-ethyl)-2-[5-(1-isocyano-2-naphthyl)-1-methyl-pyrazol-4-yl]-6H-pyrido[2,3-d]pyridazin-5-one (10 mg, 16 μmol, 18% yield) as a yellow solid. LCMS [M+1]⁺ m/z=484.2

Step 3: To a solution of 8-(1-azido-2,2-difluoro-ethyl)-2-[5-(1-isocyano-2-naphthyl)-1-methyl-pyrazol-4-yl]-6H-pyrido[2,3-d]pyridazin-5-one (10 mg, 16 μmol, 1.00 eq.) in THF (1 mL) was added 10% palladium on carbon (10 mg, 21 μmol) under nitrogen atmosphere. The suspension was degassed and purged with hydrogen for 3 times. The mixture was stirred under hydrogen (15 Psi) at 20° C. for 2 hours. The mixture was filtered and washed with DMF (1 mL). The filtrate was purified by prep-HPLC (column: Welch Xtimate C18 150×25 mm×5 μm; mobile phase: [water (HCl)-ACN]; B %: 11%-41%, 8 min). The desired fraction was collected and lyophilized to give 8-(1-amino-2,2-difluoro-ethyl)-2-[5-(1-isocyano-2-naphthyl)-1-methyl-pyrazol-4-yl]-6H-pyrido [2,3-d]pyridazin-5-one (4.8 mg, 9.4 μmol, 46% yield, HCl salt) as a white solid. LCMS [M+1]⁺ m/z=458.1; ¹H NMR (400 MHz, DMSO-d₆) δ=12.81 (s, 1H), 8.57-8.46 (m, 3H), 8.30-8.23 (m, 1H), 8.22-8.10 (m, 2H), 7.91-7.76 (m, 3H), 5.60-5.01 (m, 1H), 3.73 (d, J=2.6 Hz, 3H), 3.43-3.37 (m, 1H).

### Example 20-4

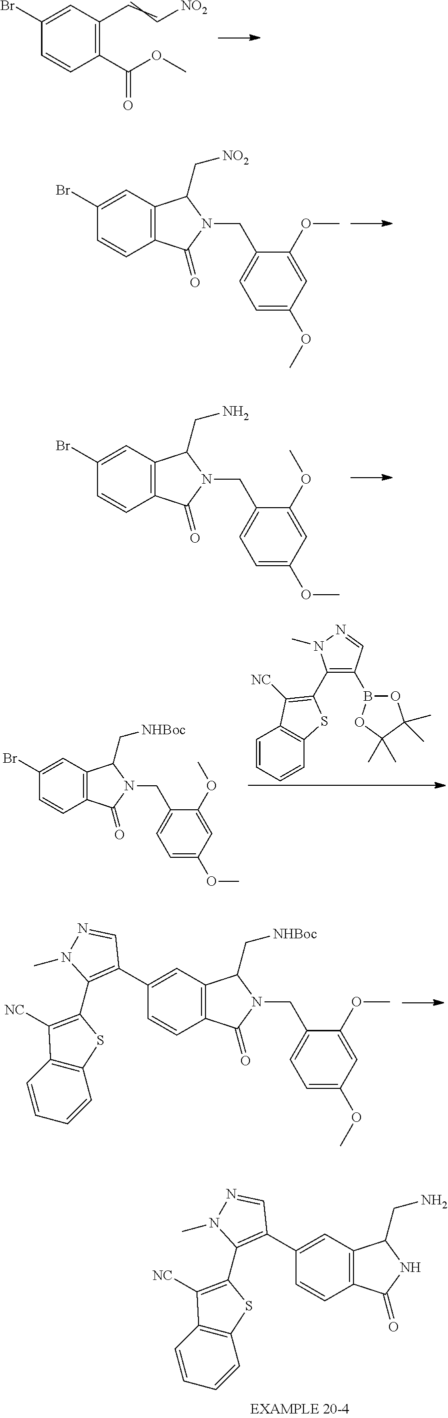

Step 1: To a solution of methyl methyl 4-bromo-2-(2-nitrovinyl)benzoate (300 mg, 1.05 mmol, 1.00 eq.) in acetonitrile (15 mL) was added sodium acetate (112 mg, 1.36 mmol, 1.30 eq.) and (2,4-dimethoxyphenyl)methanamine (210 mg, 1.26 mmol, 190 μL, 1.20 eq.). Then the mixture was stirred at 25° C. for 3 hours. The mixture was filtered and concentrated. The residue was purified by flash silica gel chromatography (SiO₂, 5-30% EtOAc/Petroleum ether gradient 100 mL/min) to afford 5-bromo-2-(2,4-dimethoxybenzyl)-3-(nitromethyl)isoindolin-1-one (280 mg, 654 μmol, 62% yield) as a light yellow solid. ¹H NMR (400 MHz, CDCl₃) δ=7.77-7.70 (m, 1H), 7.69-7.63 (m, 1H), 7.56 (s, 1H), 7.29 (s, 1H), 6.51-6.41 (m, 2H), 5.13 (d, J=14.8 Hz, 1H), 5.06 (dd, J=3.6, 13.2 Hz, 1H), 4.96 (dd, J=3.6, 7.2 Hz, 1H), 4.57 (dd, J=7.2, 13.2 Hz, 1H), 4.38 (d, J=14.8 Hz, 1H), 3.85 (s, 3H), 3.80 (s, 3H). LCMS [M+1]⁺=421.1.

Step 2: To a solution of 5-bromo-2-(2,4-dimethoxybenzyl)-3-(nitromethyl)isoindolin-1-one (280 mg, 665 μmol, 1 eq.) in ethyl alcohol (10 mL) and water (2.5 mL) was added ammonium chloride (178 mg, 3.32 mmol, 5 eq.) and ferrous powder (186 mg, 3.32 mmol, 5 eq.). Then the mixture was stirred at 80° C. for 1.5 hours. The mixture was filtered to afford 3-(aminomethyl)-5-bromo-2-(2,4-dimethoxybenzyl)isoindolin-1-one (250 mg, crude) as a yellow liquid.

Step 3: To a solution of 3-(aminomethyl)-5-bromo-2-(2,4-dimethoxybenzyl)isoindolin-1-one (250 mg, 639 μmol, 1.00 eq.) in ethyl alcohol (6 mL) was added di-tert-butyl dicarbonate (418 mg, 1.92 mmol, 3 eq.) and triethylamine (323 mg, 3.19 mmol, 445 μL, 5 eq.). The mixture was stirred at 20° C. for 2 hours. The mixture was concentrated. The residue was purified by flash silica gel chromatography (SiO₂, 10-50% EtOAc/Petroleum ether gradient 50 mL/min) to afford tert-butyl ((6-bromo-2-(2,4-dimethoxybenzyl)-3-oxoisoindolin-1-yl)methyl)carbamate (300 mg, 611 μmol, 96% yield) as a light yellow solid. ¹H NMR (400 MHz, CDCl₃) δ=7.71 (d, J=8.8 Hz, 1H), 7.63-7.56 (m, 2H), 7.30 (s, 1H), 6.49-6.40 (m, 2H), 5.06 (d, J=14.8 Hz, 1H), 4.52-4.38 (m, 2H), 4.23 (br s, 1H), 3.84 (s, 3H), 3.79 (s, 3H), 3.69 (ddd, J=3.2, 6.5, 14.4 Hz, 1H), 1.32 (s, 9H).

Step 4: To a solution of tert-butyl ((6-bromo-2-(2,4-dimethoxybenzyl)-3-oxoisoindolin-1-yl)methyl)carbamate (200 mg, 407 μmol, 1 eq.) and 2-(1-methyl-4-(4,4,5,5-tetramethyl-1,3,2-dioxaborolan-2-yl)-1H-pyrazol-5-yl)benzo[b]thiophene-3-carbonitrile (178 mg, 488 μmol, 1.20 eq.) in dioxane (8 mL) and water (1.6 mL) was added sodium carbonate (108 mg, 1.02 mmol, 2.5 eq.) and [1,1′-bis(diphenylphosphino)ferrocene]dichloropalladium(II) (29.8 mg, 40.7 umol, 0.1 eq.). The mixture was stirred at 80° C. for 3 hours under nitrogen atmosphere. The mixture was diluted with water (50 mL) and extracted with EtOAc (20 mL×3). The organic layer was washed with brine (50 mL), dried over anhydrous sodium sulfate, filtered and concentrated. The residue was purified by flash silica gel chromatography (SiO₂, 10-50% EtOAc/Petroleum ether gradient 50 mL/min) to afford tert-butyl ((6-(5-(3-cyanobenzo[b]thiophen-2-yl)-1-methyl-1H-pyrazol-4-yl)-2-(2,4-dimethoxybenzyl)-3-oxoisoindolin-1-yl)methyl)carbamate (250 mg, 385 mol, 95% yield) as a light yellow solid. ¹H NMR (400 MHz, DMSO-d₆) δ=8.29-8.17 (m, 1H), 8.01-7.94 (m, 2H), 7.71-7.60 (m, 2H), 7.57 (s, 1H), 7.51 (d, J=7.6 Hz, 1H), 7.14 (d, J=7.6 Hz, 1H), 6.99 (d, J=8.4 Hz, 1H), 6.67 (br t, J=6.0 Hz, 1H), 6.54 (d, J=2.0 Hz, 1H), 6.44 (dd, J=2.0, 8.4 Hz, 1H), 4.85 (br d, J=15.2 Hz, 1H), 4.39-4.29 (m, 2H), 3.90 (s, 3H), 3.76 (s, 3H), 3.71 (s, 3H), 1.23 (s, 9H).

Step 5: A mixture of tert-butyl ((6-(5-(3-cyanobenzo[b]thiophen-2-yl)-1-methyl-1H-pyrazol-4-yl)-2-(2,4-dimethoxybenzyl)-3-oxoisoindolin-1-yl)methyl)carbamate (120 mg, 185 μmol, 1 eq) in TFA (5 mL) was stirred at 60° C. for 4 hours. The mixture was concentrated. The residue was diluted with methanol (2 mL) and adjusted to pH=8 with aqueous ammonium hydroxide. The mixture was purified by prep-HPLC (YMC Triart 30×150 mm×7 μm; mobile phase: [water (HCl)-acetonitrile]; B %: 18/6-38%, 9 min) and lyophilized to afford 2-(4-(3-(aminomethyl)-1-oxoisoindolin-5-yl)-1-methyl-1H-pyrazol-5-yl)benzo[b]thiophene-3-carbonitrile (34 mg, 75.5 Step 1 mol, 41% yield, HCl salt) as off-white solid. ¹H NMR (400 MHz, DMSO-d₆) δ=8.61 (s, 1H), 8.29-8.24 (m, 1H), 8.21-8.03 (m, 4H), 8.02-7.95 (m, 1H), 7.76 (s, 1H), 7.72-7.62 (m, 2H), 7.56 (d, J=8.0 Hz, 1H), 7.22 (dd, J=1.2, 8.0 Hz, 1H), 4.83 (dd, J=3.2, 7.6 Hz, 1H), 3.91 (s, 3H), 3.43-3.30 (m, 1H), 3.02-2.89 (m, 1H). LCMS [M+1]⁺=400.0.

### Example 20-5

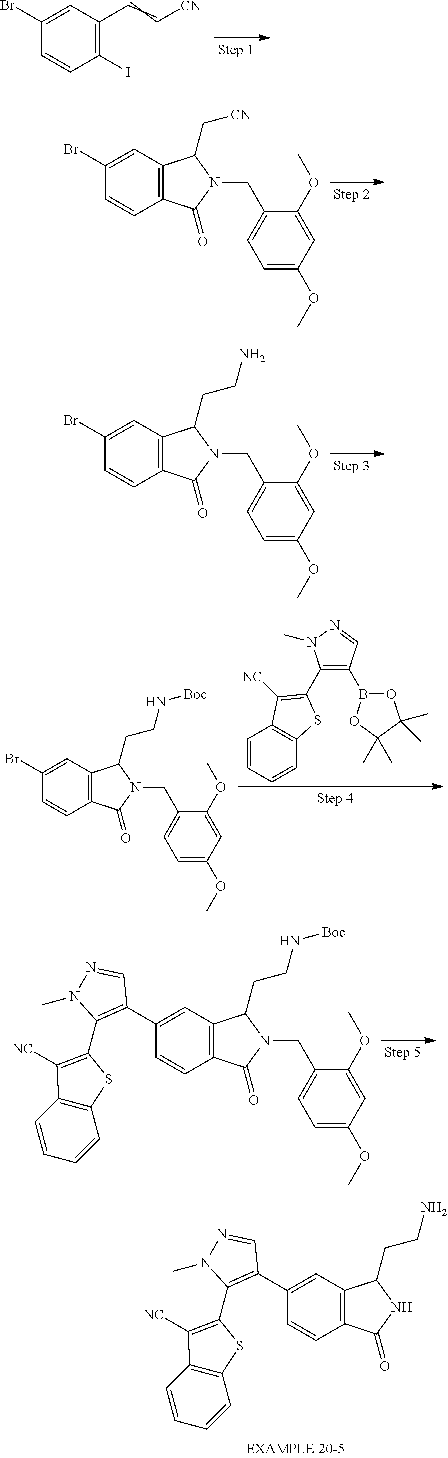

Step 1: To a mixture of 3-(5-bromo-2-iodo-phenyl)prop-2-enenitrile (2.10 g, 6.29 mmol, 1 eq.), (2,4-dimethoxyphenyl)methanamine (1.26 g, 7.55 mmol, 1.14 mL, 1.2 eq.), triphenylphosphine (330 mg, 1.26 mmol, 0.2 eq.) and cesium carbonate (4.10 g, 12.6 mmol, 2 eq.) in toluene (50 mL) was added palladium acetate (141 mg, 629 μmol, 0.10 eq.). The mixture was degassed with carbon monoxide 3 times and the resulting mixture was stirred under carbon monoxide (15 psi, a balloon) at 90° C. for 12 hours. The mixture was filtered, and the filtrate was concentrated. The residue was purified by flash silica gel chromatography (SiO₂, 30-35% EtOAc/Petroleum ether gradient 100 mL/min) to give 2-[6-bromo-2-[(2,4-dimethoxyphenyl)methyl]-3-oxo-isoindolin-1-yl]acetonitrile (620 mg, 1.45 mmol, 23% yield) as a yellow solid. ¹H NMR (400 MHz, CD₃OD-d₆) δ=7.86 (s, 1H), 7.78-7.69 (m, 2H), 7.25 (d, J=8.4 Hz, 1H), 6.56 (d, J=2.4 Hz, 1H), 6.50 (dd, J=2.4, 8.4 Hz, 1H), 5.04 (d, J=14.8 Hz, 1H), 4.66 (t, J=4.0 Hz, 1H), 4.45 (d, J=14.8 Hz, 1H), 3.84 (s, 3H), 3.78 (s, 3H). LCMS [ESI, M+1]⁺=401.0.

Step 2: To a solution of 2-[6-bromo-2-[(2,4-dimethoxyphenyl)methyl]-3-oxo-isoindolin-1-yl]acetonitrile (800 mg, 1.99 mmol, 1.00 eq.) in tetrahydrofuran (20 mL) was added borane-tetrahydrofuran (1.00 M, 3.99 mL, 2 eq.) at 0° C. under nitrogen atmosphere. The resulting mixture was stirred at 0° C. for 1 hour and then stirred at 80° C. for 2 hours. The mixture was cooled to 0° C. and quenched with methanol (20 mL) slowly at 0° C. under nitrogen atmosphere. Then the mixture was concentrated. The residue was purified by flash silica gel chromatography (SiO₂, 10-15% dichloromethane/methanol; gradient 50 mL/min) to obtain 3-(2-aminoethyl)-5-bromo-2-[(2,4-dimethoxyphenyl)methyl]isoindolin-1-one (300 mg, 691 μmol, 34% yield) as a colorless oil. LCMS [ESI, M+1]⁺=405.0.

Step 3: To a solution of 3-(2-aminoethyl)-5-bromo-2-[(2,4-dimethoxyphenyl) methyl]isoindolin-1-one (300 mg, 740 μmol, 1 eq.) in ethanol (15 mL) was added di-tert-butyl dicarbonate (323 mg, 1.48 mmol, 2 eq.). The mixture was stirred at 30° C. for 12 hours. The mixture was concentrated. The residue was purified by flash silica gel chromatography (SiO₂, 50-60% EtOAc/Petroleum ether gradient 50 mL/min) to obtain tert-butyl N-[2-[6-bromo-2-[(2,4-dimethoxyphenyl)methyl]-3-oxo-isoindolin-1-yl]ethyl]carbamate (260 mg, 499 μmol, 67% yield) as a white solid. ¹H NMR (400 MHz, CD₃Cl-d₆) δ=7.72 (d, J=8.0 Hz, 1H), 7.62-7.53 (m, 2H), 7.27 (s, 1H), 7.24 (d, J=8.0 Hz, 1H), 6.46-6.41 (m, 2H), 5.12 (d, J=14.8 Hz, 1H), 4.50-4.43 (m, 1H), 4.36 (d, J=14.8 Hz, 1H), 4.23 (br s, 1H), 3.83 (s, 3H), 3.79 (s, 3H), 2.87 (br s, 2H), 1.68 (br s, 2H), 1.42 (s, 9H). LCMS [ESI, M+1]⁺=505.0.

Step 4: To a mixture of tert-butyl N-[2-[6-bromo-2-[(2,4-dimethoxyphenyl) methyl]-3-oxo-isoindolin-1-yl]ethyl]carbamate (260 mg, 514 μmol, 1 eq.), 2-[2-methyl-4-(4,4,5,5-tetramethyl-1,3,2-dioxaborolan-2-yl)pyrazol-3-yl]benzothiophene-3-carbonitrile (225 mg, 617 μmol, 1.2 eq.) in dioxane (5 mL) and water (1 mL) was added sodium carbonate (136 mg, 1.29 mmol, 2.5 eq.) and [1,1-bis(diphenylphosphino)ferrocene]palladium (II)dichloropalladium(II) (37.6 mg, 51.4 μmol, 0.10 eq.). The mixture was degassed with nitrogen atmosphere 3 times and stirred at 80° C. for 12 hours. The mixture was diluted with water (20 mL) and extracted with EtOAc (35 mL×3). The combined organic layers were washed with brine, dried over anhydrous sodium sulfate, filtered, and concentrated. The residue was purified by flash silica gel chromatography (50% EtOAc/Petroleum ether gradient 50 mL/min) to obtain tert-butyl N-[2-[6-[5-(3-cyanobenzothiophen-2-yl)-1-methyl-pyrazol-4-yl]-2-[(2,4-dimethoxyphenyl)methyl]-3-oxo-isoindolin-1-yl]ethyl]carbamate (220 mg, 328 μmol, 64% yield) as an off-white solid. ¹H NMR (400 MHz, CD₃Cl-d₆) δ=8.00 (d, J=7.6 Hz, 1H), 7.91 (br d, J=7.6 Hz, 1H), 7.83 (s, 1H), 7.72 (br d, J=7.6 Hz, 1H), 7.66-7.53 (m, 2H), 7.27-7.20 (m, 3H), 6.47-6.37 (m, 2H), 5.10 (br d, J=14.8 Hz, 1H), 4.43-4.29 (m, 2H), 3.99 (s, 3H), 3.81 (s, 3H), 3.77 (s, 3H), 2.71 (br s, 2H), 2.21 (br s, 1H), 1.94 (br d, J=12.8 Hz, 1H), 1.42 (s, 9H). LCMS [ESI, M+1]⁺=664.4.

Step 5: A mixture of tert-butyl N-[2-[6-[5-(3-cyanobenzothiophen-2-yl)-1-methyl-pyrazol-4-yl]-2-[(2,4-dimethoxyphenyl)methyl]-3-oxo-isoindolin-1-yl]ethyl]carbamate (220 mg, 331 μmol, 1.00 eq.) in TFA (8 mL) was stirred at 60° C. for 5 hours. The mixture was concentrated. The residue was adjusted to pH=7-8 with ammonium hydroxide (5% in methanol). The mixture was diluted with methanol (5 ml) and filtered. The filtrate was purified by prep-HPLC (column: YMC Triart 30×150 mm 7 μm; mobile phase: [water (hydrochloric acid)-acetonitrile];B %: 18%-38%, 7 min), followed by lyophilization to obtain 2-[4-[3-(2-aminoethyl)-1-oxo-isoindolin-5-yl]-2-methyl-pyrazol-3-yl]benzothiophene-3-carbonitrile (65 mg, 144 μmol, 44% yield, HCl salt) as a yellow solid. ¹H NMR (400 MHz, CD₃OD-d₆) δ=8.14-8.08 (m, 1H), 7.99 (s, 1H), 7.97-7.92 (m, 1H), 7.69-7.59 (m, 4H), 7.24 (dd, J=0.8, 8.0 Hz, 1H), 4.80 (br dd, J=4.0, 6.8 Hz, 1H), 3.98 (s, 3H), 3.00-2.87 (m, 1H), 2.86-2.75 (m, 1H), 2.32 (ddd, J=1.6, 4.0, 5.6 Hz, 1H), 1.99 (td, J=2.0, 4.4 Hz, 1H). LCMS [ESI, M+1]⁺=414.2.

### Example 20-6

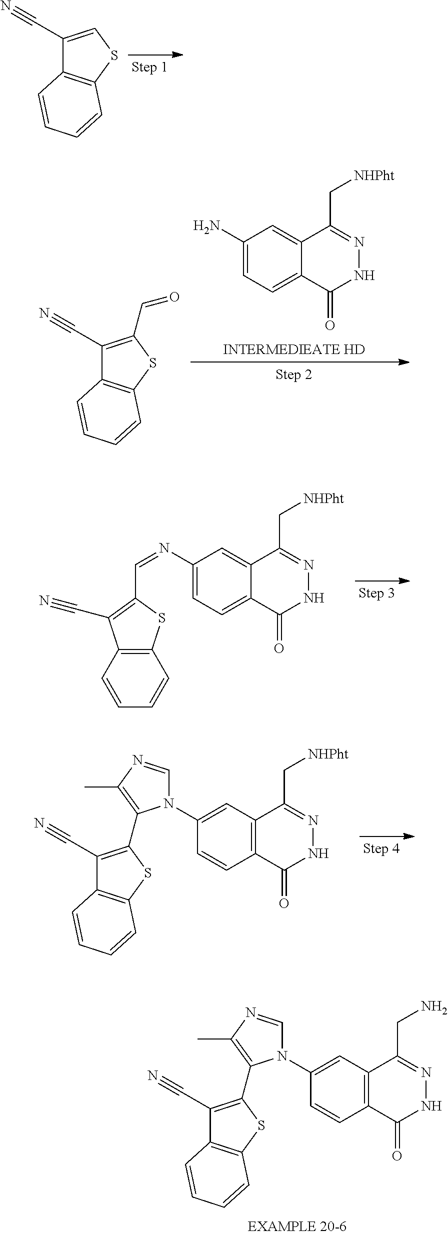

Step 1: To a solution of benzothiophene-3-carbonitrile (1.00 g, 6.28 mmol, 1.00 eq.) in THF (50 mL) at −65° C., n-BuLi (2.50 M, 2.76 mL, 1.10 eq.) was added dropwise. The reaction mixture was stirred for 1 hour at the same temperature and DMF (597 mg, 8.17 mmol, 628 uL, 1.30 eq.) was added. The resulting mixture was allowed to warm up to 20° C. and stirred for 0.5 hour. The reaction was quenched with an ammonium chloride (5 mL) at 0° C. under nitrogen and extracted with EtOAc (20 mL×2). The organic phase was dried over anhydrous sodium sulfate, filtered and concentrated under reduced pressure and the residue was purified by column chromatography (SiO₂, Petroleum ether/EtOAc 1/0 to 5/1) to give 2-formylbenzothiophene-3-carbonitrile (690 mg, 3.69 mmol, 59% yield) as a white solid. ¹H NMR (400 MHz, CDCl₃-d) 6=10.36 (s, 1H) 8.09-8.18 (m, 1H) 7.93-8.01 (m, 1H) 7.59-7.71 (m, 2H).

Step 2: To a solution of 2-[(7-amino-4-oxo-3H-phthalazin-1-yl)methyl]isoindoline-1,3-dione (50 mg, 156 μmol, 1.00 eq.) and 2-formylbenzothiophene-3-carbonitrile (59 mg, 312 μmol, 2.00 eq.) in ethyl alcohol (2 mL) was added acetic acid (47 mg, 780 μmol, 5.00 eq.). The mixture was stirred at 80° C. for 12 hours. The mixture was concentrated under reduced pressure to give 2-[[4-[(1,3-dioxoisoindolin-2-yl)methyl]-1-oxo-2H-phthalazin-6-yl]iminomethyl]benzothiophene-3-carbonitrile (50 mg, crude) as a yellow solid. The crude product was used for the next step without further purification.

Step 3: To a solution of 2-[[4-[(1,3-dioxoisoindolin-2-yl)methyl]-1-oxo-2H-phthalazin-6-yl]iminomethyl]benzothiophene-3-carbonitrile (50 mg, 102 μmol, 1.00 eq.) DMF (2 mL) was added potassium carbonate (28 mg, 204 μmol, 2.00 eq.) and 1-methyl-4-[1-(methylidyne-λ5-azanyl)ethylsulfonyl]benzene (43 mg, 204 μmol, 2.00 eq.). The mixture was stirred at 100° C. for 6 hours. The mixture was then concentrated under reduced pressure and the residue was purified by prep-HPLC (column: YMC Triart 30×150 mm×7 μm; mobile phase: [water (HCl)-ACN]; B %: 45%-65%, 9 min) to give 2-[3-[4-[(1,3-dioxoisoindolin-2-yl)methyl]-1-oxo-2H-phthalazin-6-yl]-5-methyl-imidazol-4-yl]benzothiophene-3-carbonitrile (15 mg, 27.7 μmol, two steps 12% yield) as a yellow solid. LCMS [M+1]⁺=543.3; ¹H NMR (400 MHz, MeOD-d₄) 5=9.53 (s, 1H) 8.41-8.52 (m, 2H) 7.94-8.01 (m, 2H) 7.81-7.91 (m, 5H) 7.55-7.67 (m, 2H) 5.12 (s, 2H) 2.60 (s, 3H).

Step 4: To a solution of 2-[3-[4-[(1,3-dioxoisoindolin-2-yl)methyl]-1-oxo-2H-phthalazin-6-yl]-5-methyl-imidazol-4-yl]benzothiophene-3-carbonitrile (15 mg, 28 μmol, 1.00 eq.) in ethyl alcohol (1 mL) was added hydrazine hydrate (14 mg, 276 μmol, 13.7 uL). The mixture was stirred at 80° C. for 1 hour. The mixture was filtered, and the filtrate concentrated under reduced pressure and the residue was purified by prep-HPLC (column: Phenomenex Luna C18 75 mm×30 mm×3 μm; mobile phase: [water (HCl)-ACN]; B %: 10/6-30%, 6.5 min) to give 2-[3-[4-(aminomethyl)-1-oxo-2H-phthalazin-6-yl]-5-methyl-imidazol-4-yl]benzothiophene-3-carbonitrile (5.9 mg, 12.6 μmol, 46% yield, HCl salt) as a yellow solid. LCMS [M+1]⁺=413.2; ¹H NMR (400 MHz, DMSO-d₆) δ=13.11 (s, 1H) 8.96 (s, 1H) 8.67 (br s, 3H) 8.38 (d, J=1.6 Hz, 1H) 8.24 (d, J=8.8 Hz, 1H) 8.09 (d, J=8.0 Hz, 1H) 7.94 (d, J=8.0 Hz, 1H) 7.52-7.73 (m, 3H) 4.40 (br d, J=5.6 Hz, 2H) 2.42 (s, 3H).

### Example 20-7

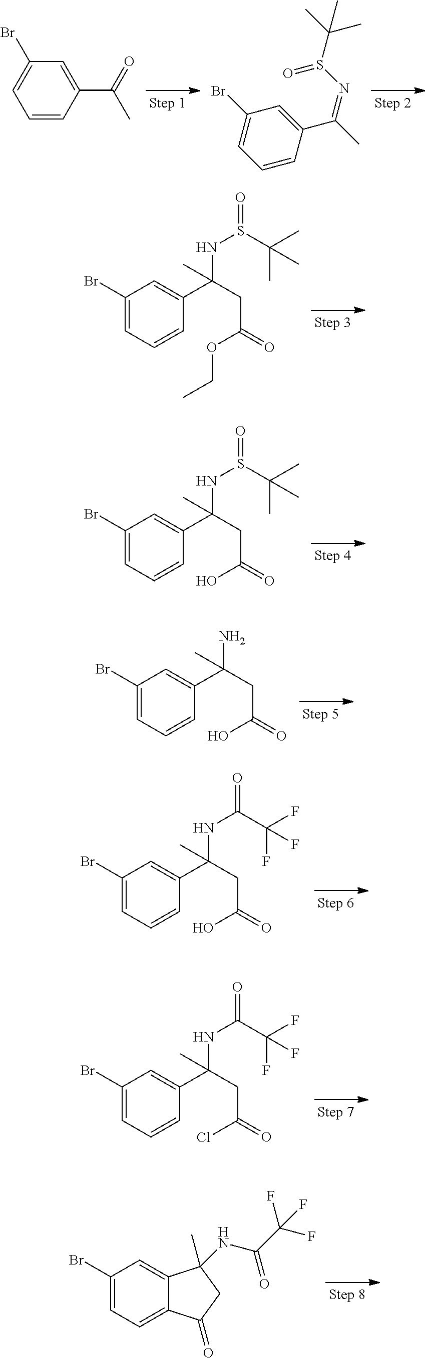

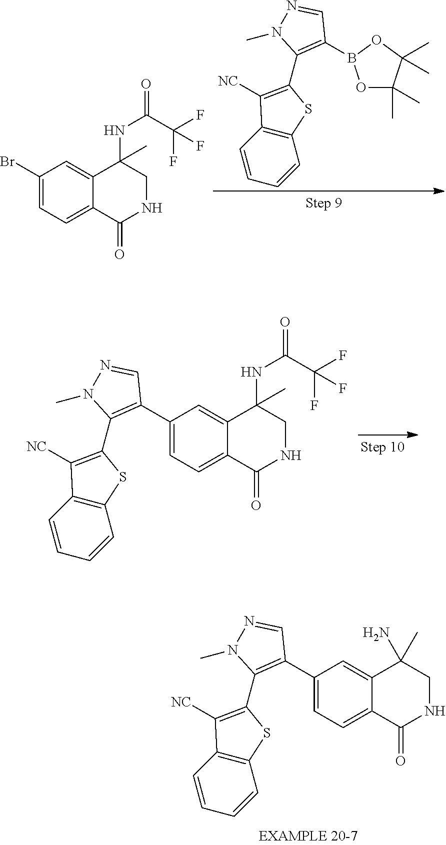

Step 1: To a solution of 1-(3-bromophenyl)ethanone (20.0 g, 100 mmol, 13.3 mL) and 2-methylpropane-2-sulfinamide (13.4 g, 111 mmol, 1.10 eq.) in THF (350 mL) was added titanium tetra-isopropoxide (57.1 g, 201 mmol, 59.3 mL). The mixture was stirred at 70° C. for 18 hours. The reaction mixture was cooled to 20° C. and poured onto brine (300 mL) and filtered. The filtrate was extracted with EtOAc (600 mL) and dried over sodium sulfate, filtered and concentrated under vacuum. The residue was purified by column chromatography (SiO₂, petroleum ether/EtOAc 20/1 to 5/1) to give (Z)—N-(1-(3-bromophenyl)ethylidene)-2-methylpropane-2-sulfinamide (17.0 g, 56.3 mmol, 56% yield) as a yellow solid. ¹H NMR (400 MHz, CDCl₃) δ=8.00 (s, 1H), 7.79 (d, J=8.0 Hz, 1H), 7.61 (d, J=8.0 Hz, 1H), 7.30 (t, J=8.0 Hz, 1H), 2.75 (s, 3H), 1.33 (s, 9H).

Step 2: A solution of zinc (8.65 g, 132 mmol, 10.0 eq.) and copper(I)chloride (1.57 g, 15.9 mmol, 1.20 eq.) in THF (50 mL) was refluxed for 0.5 hour. The reaction mixture was cooled to 20° C. and ethyl 2-bromoacetate (5.53 g, 33.1 mmol, 3.66 mL, 2.50 eq.) was added dropwise and the reaction mixture was allowed to stir at 50° C. for 0.75 hour. The reaction mixture was cooled to 0° C. then a solution of (Z)—N-(1-(3-bromophenyl)ethylidene)-2-methylpropane-2-sulfinamide (4.00 g, 13.2 mmol, 1.00 eq.) in THF (50 mL) was added and the reaction mixture was stirred at 20° C. for 0.5 hour. The mixture was then quenched with saturated sodium bicarbonate and filtered through a bed of celite. The filtrate was extracted with EtOAc (300 mL) and washed with brine (100 mL), dried over sodium sulfate, filtered and concentrated under reduced pressure. The residue was purified by column chromatography (SiO₂, petroleum ether/EtOAc 10/1 to 1/1) to get ethyl 3-(3-bromophenyl)-3-(tert-butylsulfinylamino)butanoate (5.0 g, 12.8 mmol, 97% yield) as a yellow gum. ¹H NMR (400 MHz, CDCl₃) δ=7.57 (d, J=2.0 Hz, 1H), 7.42-7.36 (m, 1H), 7.36-7.31 (m, 1H), 7.23-7.17 (m, 1H), 5.54 (s, 1H), 4.12-4.03 (m, 2H), 3.12-2.99 (m, 2H), 1.74 (s, 3H), 1.31 (s, 9H), 1.19 (t, J=7.2 Hz, 3H).

Step 3: To a solution of ethyl 3-(3-bromophenyl)-3-(tert-butylsulfinylamino)butanoate (2.00 g, 5.12 mmol, 1.00 eq.) in methanol (16 mL) and water (4 mL) was added lithium hydroxide hydrate (688 mg, 16.4 mmol, 3.20 eq.). The mixture was stirred at 30° C. for 2 hours. The reaction mixture was then concentrated under reduced pressure and quenched with saturated solution of ammonium chloride (40 mL). The reaction mixture was acidified with hydrochloric acid (1.00 M) and the precipitated solid was filtered, washed with water and dried to get 3-(3-bromophenyl)-3-(tert-butylsulfinylamino)butanoic acid (1.60 g, 4.42 mmol, 86% yield) as a light yellow solid. ¹H NMR (400 MHz, DMSO-d₆) δ=12.44 (s, 1H), 7.64-7.58 (m, 1H), 7.50-7.40 (m, 2H), 7.34-7.25 (m, 1H), 5.60 (s, 1H), 3.10 (d, J=16.4 Hz, 1H), 2.84 (d, J=16.4 Hz, 1H), 1.67 (s, 3H), 1.15 (s, 9H).

Step 4: A solution of 3-(3-bromophenyl)-3-(tert-butylsulfinylamino)butanoic acid (1.00 g, 2.76 mmol, 1.00 eq.) in TFA (20 mL) was allowed to stir at 25° C. for 36 hours. The reaction mixture was concentrated under reduced pressure and quenched with saturated solution of ammonium chloride (45 mL). The reaction mixture was acidified with hydrochloric acid (1.00 M) and the precipitated solid was filtered, washed with water and dried to get 3-amino-3-(3-bromophenyl)butanoic acid (712 mg, crude) as a yellow oil. LCMS [M+1]⁺ 258.0.

Step 5: A solution of 3-amino-3-(3-bromophenyl)butanoic acid (712 mg, 2.76 mmol, 1.00 eq.) in trifluoroacetic anhydride (20 mL) was allowed to stir at 20° C. for 36 hours. The reaction mixture was concentrated under reduced pressure to get a residue. The residue was purified by HPLC (TFA conditions) to get 3-(3-bromophenyl)-3-[(2,2,2-trifluoroacetyl)amino]butanoic acid (520 mg, 1.46 mmol, 53% yield) as a light yellow solid. ¹H NMR (400 MHz, CDCl₃) δ=7.70 (s, 1H), 7.51-7.40 (m, 2H), 7.27-7.25 (m, 1H), 3.16 (d, J=16.0 Hz, 1H), 2.96 (d, J=16.0 Hz, 1H), 1.90 (s, 3H).

Step 6: A solution of 3-(3-bromophenyl)-3-[(2,2,2-trifluoroacetyl)amino]butanoic acid (150 mg, 424 umol, 1.00 eq.) in thionyl dichloride (4.0 mL) was allowed to stir at 80° C. for 3 hours. The reaction mixture was concentrated under reduced pressure to get 3-(3-bromophenyl)-3-[(2,2,2-trifluoroacetyl)amino]butanoyl chloride (157 mg, crude) as a light yellow solid.

Step 7: A solution of 3-(3-bromophenyl)-3-[(2,2,2-trifluoroacetyl)amino]butanoyl chloride (157 mg, 421 μmol, 1 eq.) in DCM (4 mL) was added aluminum chloride (112 mg, 843 umol, 2 eq.) in a portionwise fashion at 0° C. The reaction mixture was stirred at 20° C. for 12 hours. The reaction mixture was then concentrated under reduced pressure and the residue purified by prep-TLC (SiO₂, petroleum ether/EtOAc 3/1) to get N-(6-bromo-1-methyl-3-oxo-indan-1-yl)-2,2,2-trifluoro-acetamide (63 mg, 184 μmol, 44% yield) as a light-yellow solid. ¹H NMR (400 MHz, DMSO-d₆) δ=9.84 (s, 1H), 7.87 (d, J=1.6 Hz, 1H), 7.71 (dd, J=1.6, 8.0 Hz, 1H), 7.57 (d, J=8.4 Hz, 1H), 3.07 (d, J=18.6 Hz, 1H), 2.80 (d, J=18.6 Hz, 1H), 1.69 (s, 3H).

Step 8: A solution of N-(6-bromo-1-methyl-3-oxo-indan-1-yl)-2,2,2-trifluoro-acetamide (60 mg, 179 μmol) in DCM (2 mL) was added 4-methylbenzenesulfonic acid (172 mg, 1.79 mmol, 127 μL, 10.0 eq.) and the reaction mixture was cooled to 0° C. Then to the reaction mixture was added sodium azide (35 mg, 536 μmol, 3.00 eq.) at 0° C. and stirred at 20° C. for 16 hours. The reaction was basified with sodium hydroxide (20 mL, 2.0 M) and extracted with EtOAc (60 mL). The organic layer was separated, washed with brine (30 mL), dried over anhydrous sodium sulfate and concentrated to give N-(6-bromo-4-methyl-1-oxo-2,3-dihydroisoquinolin-4-yl)-2,2,2-trifluoro-acetamide (21 mg, 60 μmol, 34% yield) as an off-white solid. ¹H NMR (400 MHz, DMSO-d₆) δ=9.58 (s, 1H), 8.15 (s, 1H), 7.82 (d, J=8.4 Hz, 1H), 7.66 (dd, J=2.0, 8.4 Hz, 1H), 7.56 (d, J=2.0 Hz, 1H), 4.00 (dd, J=2.8, 12.8 Hz, 1H), 3.23 (dd, J=2.8, 12.8 Hz, 1H), 1.68 (s, 3H).

Step 9: A mixture of N-(6-bromo-4-methyl-1-oxo-2,3-dihydroisoquinolin-4-yl)-2,2,2-trifluoro-acetamide (20 mg, 57 μmol), 2-[2-methyl-4-(4,4,5,5-tetramethyl-1,3,2-dioxaborolan-2-yl)pyrazol-3-yl]benzothiophene-3-carbonitrile (39 mg, 85 μmol), [2-(2-aminophenyl)phenyl]palladium(1+);bis(1-adamantyl)-butyl-phosphane;methanesulfonate (8.30 mg, 11.4 μmol, 0.2 eq.) and sodium carbonate (18 mg, 171 μmol, 3.00 eq.) in dioxane (2 mL) and water (0.4 mL) was degassed and purged with nitrogen 3 times, then stirred at 80° C. for 12 hours. The reaction mixture was diluted with water (50 mL) and extracted with EtOAc (120 mL) and the organic layer was washed with brine (100 mL), dried over sodium sulfate, filtered and concentrated under reduced pressure. The residue was purified by prep-TLC (SiO₂, petroleum ether/EtOAc=1/1) to give 2,2,2-trifluoro-N-[6-[5-(3-isocyanobenzothiophen-2-yl)-1-methyl-pyrazol-4-yl]-4-methyl-1-oxo-2,3-dihydroisoquinolin-4-yl]acetamide (26 mg, 50 μmol, 89% yield) as a red solid. LCMS [M+1]⁺510.2.

Step 10: To a solution of 2,2,2-trifluoro-N-[6-[5-(3-isocyanobenzothiophen-2-yl)-1-methyl-pyrazol-4-yl]-4-methyl-1-oxo-2,3-dihydroisoquinolin-4-yl]acetamide (26 mg, 51 μmol) in dioxane (2 mL) was added a solution of lithium hydroxide hydrate (21 mg, 510 μmol, 10.0 eq.) in water (0.5 mL). The reaction mixture was stirred at 50° C. for 12 hours. The reaction mixture was then diluted with water (20 mL) and extracted with 2-methyl tetrahydrofuran (40 mL). The organic layer was washed with brine (20 mL), dried over sodium sulfate, filtered and concentrated under reduced pressure. The residue was purified by prep-HPLC (Welch Xtimate C18 150×25 mm×5 μm; mobile phase: [water (0.05% HCl)-ACN]; B %: 15%-45%, 10 min) to give 2-(4-(4-amino-4-methyl-1-oxo-1,2,3,4-tetrahydroisoquinolin-6-yl)-1-methyl-1H-pyrazol-5-yl)benzo[b]thiophene-3-carbonitrile (8 mg, 18 μmol, 35% yield, HCl salt) as a yellow solid. ¹H NMR (400 MHz, MeOD-d₄) δ=8.16-8.09 (m, 1H), 8.07 (s, 1H), 8.03-7.97 (m, 2H), 7.73-7.62 (m, 2H), 7.55-7.50 (m, 1H), 7.50-7.40 (m, 1H), 3.97 (s, 31H), 3.68 (d, J=14.0 Hz, 1H), 3.53 (d, J=14.0 Hz, 1H), 1.50 (s, 3H).

### Examples 21-1 and 21-2

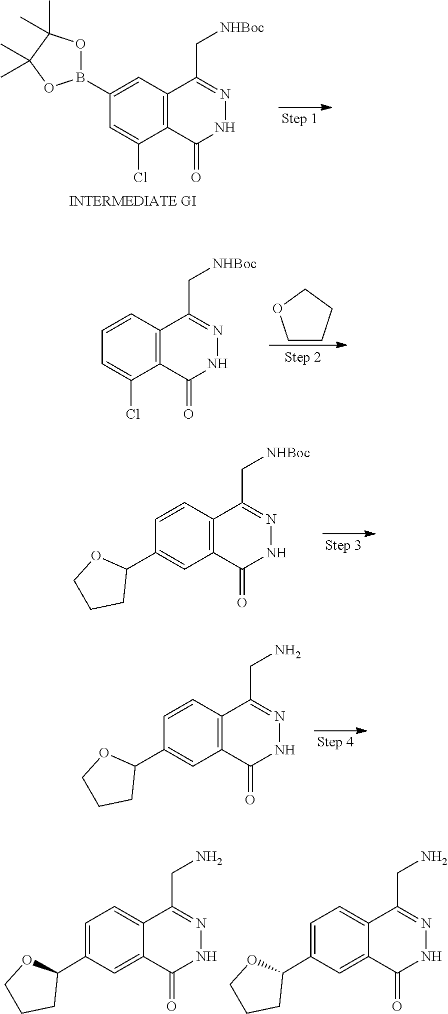

Step 1: To a solution of tert-butyl N-[[5-chloro-4-oxo-7-(4,4,5,5-tetramethyl-1,3,2-dioxaborolan-2-yl)-3H-phthalazin-1-yl]methyl]carbamate (1.00 g, 2.30 mmol, 1.0 eq.) and triethylamine (2.30 mmol, 0.32 mL) in ethanol (10 mL) and water (10 mL) was added AgNO₃ (196 mg, 1.15 mmol, 0.50 eq.). The mixture was stirred at 80° C. for 1 hour. The reaction mixture was then quenched with sat. aq. NH₄Cl (50 mL) at 0° C., and then diluted with water (10 mL) and extracted with EtOAc (25 mL×2). The combined organic layers were washed with brine (20 mL), dried over anhydrous sodium sulfate, filtered and concentrated under reduced pressure. The residue was purified by column chromatography (SiO₂, Petroleum ether/EtOAc 1/0 to 1/2) to give tert-butyl ((5-chloro-4-oxo-3,4-dihydrophthalazin-1-yl)methyl)carbamate (660 mg, 2.02 mmol, 88% yield) as a white solid. LCMS [M+1]⁺=310.0; ¹H NMR (400 MHz, DMSO-d₆) δ=12.55 (s, 1H), 7.98-7.93 (m, 1H), 7.88-7.85 (m, 2H), 7.33 (t, J=5.2 Hz, 1H), 4.40 (d, J=5.6 Hz, 2H), 1.38 (s, 9H).

Step 2: A mixture of tert-butyl N-[(5-chloro-4-oxo-3H-phthalazin-1-yl)methyl]carbamate (120 mg, 0.39 mmol, 1.00 eq.), THF (3.10 mmol, 251 μL, 8.00 eq.), dibromonickel trihydrate (6.3 mg, 23 μmol, 0.06 eq.), 4,7-dimethoxy-1,10-phenanthroline (5.6 mg, 23 μmol, 0.06 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (4.4 mg, 4 μmol, 0.01 eq.), quinuclidin-3-yl acetate (72 mg, 426 μmol, 1.10 eq.) in DMSO (4 mL) and water (0.3 mL). The vial was sealed and placed under nitrogen. The reaction was stirred and irradiated with a 10 W blue LED lamp (3 cm away), with cooling water to keep the reaction temperature at 25° C. for 12 hours. The residue was diluted with water (15 mL) and extracted with EtOAc (15 mL×3). The combined organic layers were washed with brine (30 mL), dried over anhydrous sodium sulfate, filtered and concentrated. The residue was purified by flash chromatography (SiO₂, 0-40% EtOAc/Petroleum ether) to give tert-butyl ((4-oxo-6-(tetrahydrofuran-2-yl)-3,4-dihydrophthalazin-1-yl)methyl)carbamate (40 mg, 92 μmol, 6% yield) as a light yellow solid: LCMS [M+1]⁺=346.2.

Step 3: To a solution of tert-butyl N-[(4-oxo-5-tetrahydrofuran-2-yl-3H-phthalazin-1-yl)methyl]carbamate (60 mg, 174 μmol, 1.00 eq.) in dichloromethane (1.5 mL) was added HCl/dioxane (4.0 M, 1.50 mL). The mixture was stirred at 25° C. for 1 hour. The reaction mixture was concentrated and the residue was purified by prep-HPLC (Phenomenex C18 150×25 mm×10 μm; mobile phase: [water (ammonia hydroxide v/v)-MeCN]; B %: 3%-33%, 8 min) to give (R,S)-4-(aminomethyl)-7-(tetrahydrofuran-2-yl)phthalazin-1 (2H)-one (40 mg, 157 μmol, 90% yield) as a light yellow gum. LCMS [M+1]⁺=246.3.

Step 4: (R,S)-4-(aminomethyl)-7-(tetrahydrofuran-2-yl)phthalazin-1 (2H)-one (40.0 mg, 163 umol, 1.00 eq.) enantiomers were separated by chiral SFC (DAICEL CHIRALPAK IG (250 mm×30 mm×10 μm); mobile phase: [0.1% NH₃H₂O MeOH]; B %: 45%-45%, 6.1; 35 min) to give (R)-4-(aminomethyl)-7-(tetrahydrofuran-2-yl)phthalazin-1 (2H)-one, EXAMPLE 21-1 (13 mg, 49 μmol, 30% yield, 97.6% ee, Formic Acid salt) as a yellow solid. LCMS [M+H]⁺=246.2; ¹H NMR (400 MHz, DMSO-d₆) δ=8.55 (s, 4H), 8.34 (s, 1H), 7.98-7.90 (m, 2H), 5.08 (t, J=7.2 Hz, 1H), 4.18-4.13 (m, 3H), 4.02-3.96 (m, 1H), 2.52-2.45 (m, 1H), 2.10-2.03 (m, 2H), 1.84-1.76 (m, 1H) and (S)-4-(aminomethyl)-7-(tetrahydrofuran-2-yl)phthalazin-1 (2H)-one, EXAMPLE □□□□□ (9.5 mg, 31 μmol, 21% yield, 98.2% ee, Formic Acid salt) as a yellow solid; LCMS [M+1]⁺=246.1. ¹H NMR (400 MHz, DMSO-d₆) δ=8.55 (s, 2H), 8.35 (s, 1H), 7.97-7.91 (m, 2H), 5.09 (t, J=7.2 Hz, 1H), 4.22-4.13 (m, 3H), 4.02-3.96 (m, 1H), 2.51-2.46 (m, 1H), 2.10-2.03 (m, 2H), 1.84-1.76 (m, 1H).

### Example 21-3

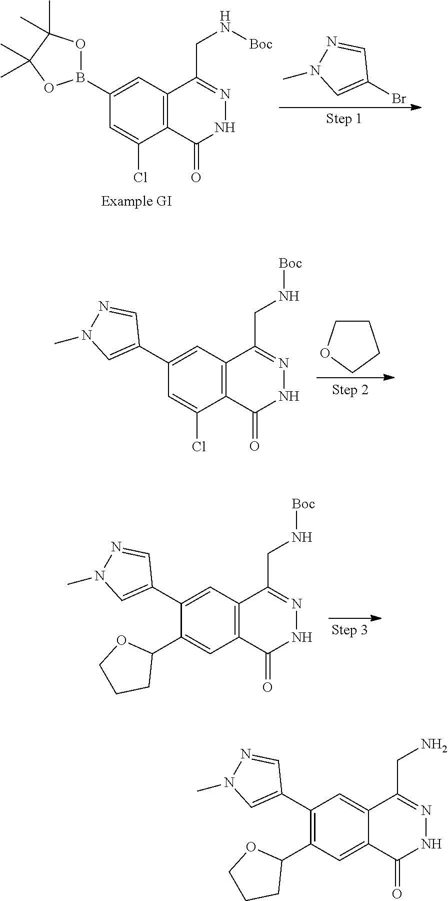

Step 1: A mixture of tert-butylN-[[5-chloro-4-oxo-7-(4,4,5,5-tetramethyl-1,3,2-dioxaborolan-2-yl)-3H-phthalazin-1-yl]methyl]carbamate (300 mg, 689 μmol, 1.00 eq.), 4-bromo-1-methyl-pyrazole (122 mg, 757 μmol, 1.10 eq.), [2-(2-aminophenyl)phenyl]palladium(1+);bis(1-adamantyl)-butyl-phosphane;methanesulfonate (50 mg, 69 μmol, 0.1 eq.), potassium carbonate (190 mg, 1.38 mmol, 2.00 eq.) in dioxane (5 mL) and water (0.5 mL) was degassed and purged with nitrogen for 3 times, and then the mixture was stirred at 90° C. for 2 hours under nitrogen atmosphere. The mixture was filtered and concentrated in vacuum to give a residue, which was purified by column chromatography (SiO₂, Petroleum ether/EtOAc=5/1 to 2/1) to give tert-butyl N-[[5-chloro-7-(1-methylpyrazol-4-yl)-4-oxo-3H-phthalazin-1-yl]methyl]carbamate (70 mg, 179 μmol, 26% yield) as a gray solid. ¹H NMR (400 MHz, DMSO-dc) δ=12.43 (s, 1H), 8.45 (s, 1H), 8.18-8.07 (m, 3H), 7.44 (t, J=6.0 Hz, 1H), 4.44 (d, J=6.4 Hz, 2H), 3.89 (s, 3H), 1.39 (s, 9H).

Step 2: To a 15 mL of vial equipped with a stir bar were added tert-butyl N-[[5-chloro-7-(1-methylpyrazol-4-yl)-4-oxo-3H-phthalazin-1-yl]methyl]carbamate (80 mg, 205 μmol, 1.00 eq.), tetrahydrofuran (410 μmol, 33.3 μL, 2.00 eq.), quinuclidin-3-yl acetate (35 mg, 205 μmol, 1.00 eq.), Ir[dF(CF₃)ppy]2(dtbpy)(PF₆) (2.3 mg, 2 μmol, 0.01 eq.). NiBr₂·3H₂O (53 mg, 205 μmol, 1.00 eq.), water (148 mg, 8.21 mmol, 148 μL, 40.0 eq.) and 4,7-dimethoxy-1,10-phenanthroline (0.5 mg, 2.1 μmol, 0.01 eq.) in DMSO (4 mL). The vial was sealed and placed under nitrogen. The reaction was stirred and irradiated with a 10 W blue LED lamp (3 cm away), with cooling water to keep the reaction temperature at 25° C. for 3 hours. The reaction mixture was then poured into water (10 mL) and extracted with EtOAc (30 mL×3). The combined organic phase was dried, filtered and concentrated to give the residue, which was purified by prep-HPLC (Phenomenex Luna C18 100×30 mm×5 μm; mobile phase: [water (FA)-ACN]; B %: 20/6-50%, 8 min) to give tert-butyl ((7-(1-methyl-1H-pyrazol-4-yl)-4-oxo-6-(tetrahydrofuran-2-yl)-3,4-dihydrophthalazin-1-yl)methyl)carbamate (20 mg, 45 μmol, 22% yield) as a white solid. LCMS [ESI, M+1]⁺: 426.2.

Step 3: A mixture of tert-butyl N-[[7-(1-methylpyrazol-4-yl)-4-oxo-5-tetrahydrofuran-2-yl-3H-phthalazin-1-yl]methyl]carbamate (20 mg, 47 μmol, 1 eq.), HCl/dioxane (4 M, 12 uL, 1 eq.) in DCM (2 mL) was degassed and purged with N₂ for 3 times, and then the mixture was stirred at 25° C. for 1 h under N₂ atmosphere. The mixture was concentrated in vacuum to give a residue. The residue was purified by prep-HPLC (Phenomenex Luna C18 100×30 mm×5 μm; mobile phase: [water (FA)-ACN]; B %: 1%-18%, 8 min) to give 4-(aminomethyl)-6-(1-methyl-1H-pyrazol-4-yl)-7-(tetrahydrofuran-2-yl)phthalazin-1 (2H)-one (5 mg, 14 μmol, 30% yield) as a white solid. LCMS [ESI, M+1]⁺: 326.0; ¹H NMR (400 MHz, DMSO-d₆) δ=12.69-12.33 (m, 1H), 8.36 (s, 1H), 8.33 (s, 1H), 8.09 (s, 1H), 7.90 (s, 1H), 7.80 (s, 1H), 5.20 (t, J=7.2 Hz, 1H), 4.13 (t, J=6.4 Hz, 3H), 3.93 (s, 3H), 3.86-3.81 (m, 1H), 2.32 (dd, J=12.4, 6.8 Hz, 1H), 2.01-1.89 (m, 2H), 1.67-1.55 (m, 1H).

### Example 21-4

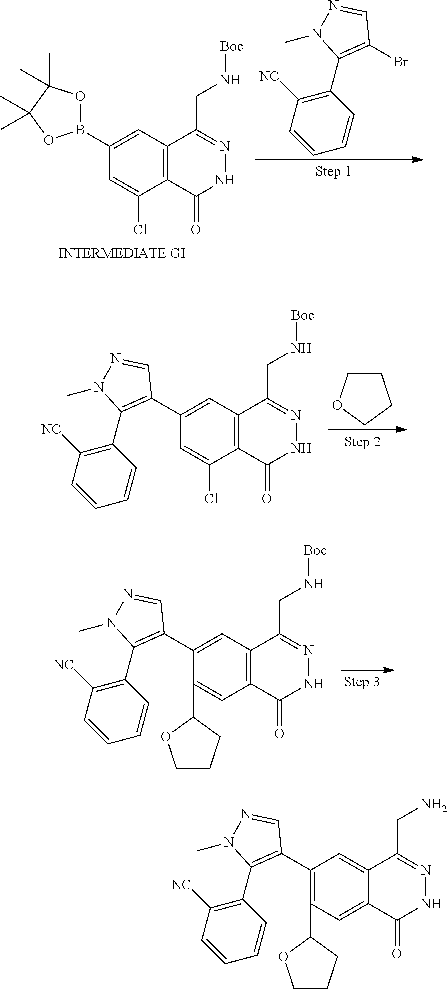

EXAMPLE 21-4 was prepared using the same 3 step method used for the preparation of EXAMPLE 20-3 starting from INTERMEDIATE GI (500 mg, 1.15 mmol, 1.00 eq.) and 2-(4-iodo-2-methyl-pyrazol-3-yl)benzonitrile (390 mg, 1.26 mmol, 1.10 eq.) to give 2-(4-(4-(aminomethyl)-1-oxo-7-(tetrahydrofuran-2-yl)-1,2-dihydrophthalazin-6-yl)-1-methyl-11H-pyrazol-5-yl)benzonitrile (10.0 mg, 22.9 umol, 21.9% yield, 97.5% purity, FA) as an off-white solid. LCMS (ESI, M+1): m/z=427.1. ¹H NMR (400 MHz, 25° C., DMSO-d₆) δ=12.46 (s, 1H), 8.32 (s, 1H), 8.26 (d, J=8.4 Hz, 1H), 8.13-8.08 (m, 1H), 8.00-7.94 (m, 1H), 7.93-7.74 (m, 3H), 7.71-7.55 (m, 2H), 7.49 (d, J=1.2 Hz, 1H), 5.00-4.82 (m, 1H), 4.07-3.98 (m, 1H), 3.79-3.71 (m, 6H), 3.63 s, 3H), 2.05-1.46 (m, 2H); ¹H NMR (400 MHz, 80° C., DMSO-d₆) δ=12.16 (s, 1H), 8.30 (s, 1H), 8.22 (s, 1H), 8.06 (d, J=7.6 Hz, 1H), 7.99-7.91 (m, 1H), 7.87-7.70 (m, 3H), 7.68-7.56 (m, 2H), 7.53 (d, J=1.6 Hz, 1H), 5.03-4.85 (m, 1H), 4.09-4.00 (m, 1H), 3.78 (s, 2H), 3.73 (s, 3H), 3.63 (s, 2H), 2.52-2.51 (m, 2H), 2.17-1.49 (m, 2H).

### Examples 21-5 and 21-6

EXAMPLES 21-5 and 21-6 were prepared from Intermediate 15-1 and oxetane using the same procedure as Steps 2, 3 and 4 for EXAMPLES 21-1 and 21-2.

TABLE 21

| Example | Structure | Compound Name |
|---|---|---|
| 21-5 | 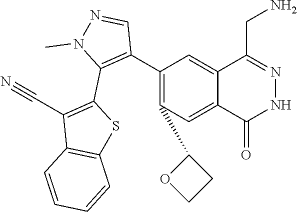 [SMILES: Cn1ncc(-c2cc3c(CN)n[nH]c(=O)c3cc2[C@@H]2CCO2)c1-c1sc2ccccc2c1C#N] | (S)-2-(4-(4-(aminomethyl)-7-(oxetan-2-yl)-1-oxo- 1,2-dihydrophthalazin-6-yl)-1-methyl-1H-pyrazol- 5-yl)benzo[b]thiophene-3-carbonitrile LCMS [M + H]⁺ 469.3; ¹H NMR (400 MHz, MeOD-d₄) δ = 8.79 (s, 1H), 8.04-7.99 (m, 1H), 7.91 (dd, J = 1.6, 7.2 Hz, 1H), 7.78 (s, 1H), 7.71 (s, 1H), 7.58 (dt, J = 1.2, 7.6 Hz, 2H), 5.97 (s, 1H), 4.81-4.76 (m, 1H), 4.68-4.62 (m, 1H), 4.04 (s, 3H), 3.93 (s, 2H), 2.94-2.79 (m, 1H), 2.62-2.47 (m, 1H) |
| 21-6 | 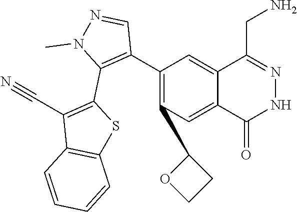 [SMILES: Cn1ncc(-c2cc3c(CN)n[nH]c(=O)c3cc2[C@H]2CCO2)c1-c1sc2ccccc2c1C#N] | (R)-2-(4-(4-(aminomethyl)-7-(oxetan-2-yl)-1-oxo- 1,2-dihydrophthalazin-6-yl)-1-methyl-1H-pyrazol- 5-yl)benzo[b]thiophene-3-carbonitrile LCMS [M + H]⁺ 469.2; ¹H NMR (400 MHz, MeOD-d₄) δ = 8.79 (s, 1H), 8.03-7.99 (m, 1H), 7.91 (, J = 1.6, 7.2 Hz, 1H), 7.8(s, 1H), 7.71 (s, 1H), 7.58 (d, q, J = 1.2, 7.6 Hz, 2H), 5.97 (t, J = 7.6 Hz, 1H), 4.81- 4.76 (m, 1H), 4.68-4.62 (m, 1H), 4.04 (s, 3H), 3.92 (s, 2H), 2.95-2.76 (m, 1H), 2.63-2.47 (m, 1H) |

### Example 21-7 Example 21-8

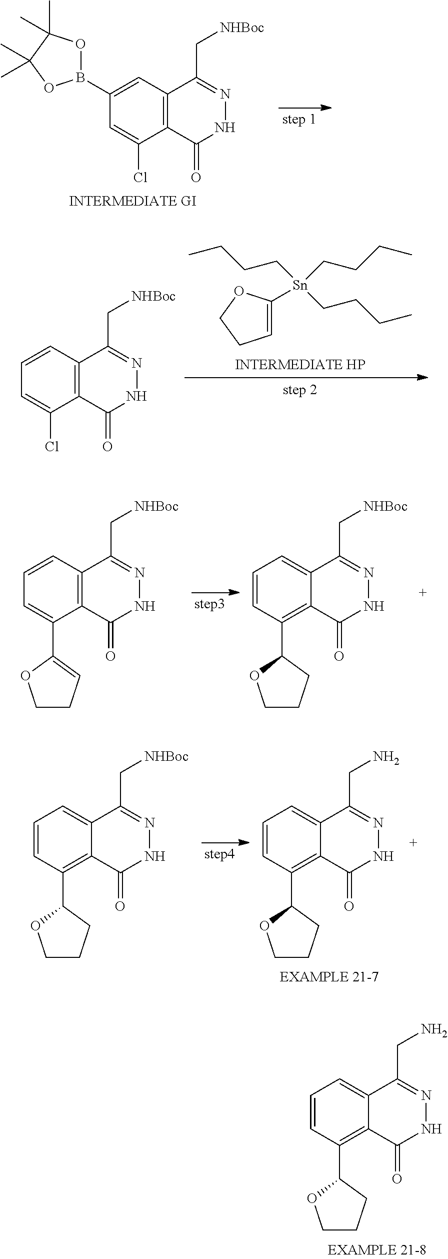

Step 1: To a solution of INTERMEDIATE GI (0.30 g, 688 umol, 1.0 eq.) and triethylamine (69.7 mg, 688 umol, 95.8 uL, 1.0 eq.) in ethyl alcohol (3.0 mL) and water (3.0 mL) was added AgNO₃ (234 mg, 1.38 mmol, 2.0 eq.). The mixture was stirred at 80° C. for 1 hour. The reaction mixture was quenched by addition of sat.aq. ammonium chloride (10 mL) at 0° C., and then diluted with water (10 mL) and extracted with ethyl acetate (25 mL×2). The combined organic layers were washed with brine (20 mL), dried over anhydrous sodium sulfate, filtered and concentrated to give tert-butyl ((5-chloro-4-oxo-3,4-dihydrophthalazin-1-yl)methyl)carbamate (210 mg, 677 umol, 98% yield) as a white solid. LCMS [M+H]⁺: 310.1. Step 2: To a mixture of tert-butyl ((5-chloro-4-oxo-3,4-dihydrophthalazin-1-yl)methyl)carbamate (67 mg, 216 umol, 1.0 eq.) and INTERMEDIATE HP (155 mg, 433 umol, 2 eq.) in N,N-dimethylacetamide (0.5 mL) was added cataCXium-Pd G3 (15.7 mg, 21.6 umol, 0.1 eq.) in one portion. The mixture was stirred at 80° C. for 2 hours. The reaction mixture was diluted with water (20 mL) and extracted with ethyl acetate (3×30 mL). The combined organic layers were washed with brine (100 mL), dried over sodium sulfate, filtered and concentrated under reduced pressure to give a residue. The residue was purified by reversed phase flash [water (0.1% formic acid)/acetonitrile)], further purified by prep-HPLC (column: Phenomenex luna C18 150×25 mm 10 μm; mobile phase: [water (0.1% formic acid)-acetanitrile];B %: 20%-50%, 10 min) to give tert-butyl ((5-(4,5-dihydrofuran-2-yl)-4-oxo-3,4-dihydrophthalazin-1-yl)methyl)carbamate (70.0 mg, 203 umol, 47% yield, 99.0% purity) as a white solid.

LCMS [M+H]⁺: 344.1. ¹H NMR (400 MHz, CHLOROFORM-d) δ=10.43-10.32 (m, 1H), 8.07-7.97 (m, 1H), 7.92 (t, J=7.6 Hz, 1H), 7.57 (d, J=7.6 Hz, 1H), 5.36 (br s, 1H), 4.70 (br d, J=4.4 Hz, 2H), 3.88 (t, J=5.6 Hz, 2H), 2.12-2.05 (m, 2H), 1.49 (s, 9H).

Step 3: To a solution of tert-butyl ((5-(4,5-dihydrofuran-2-yl)-4-oxo-3,4-dihydrophthalazin-1-yl)methyl)carbamate 30.0 mg, 87.3 umol, 1 eq.) in ethyl acetate (0.30 mL) was added palladium on carbon (30 mg, 10% purity) under nitrogen. The suspension was degassed and purged with hydrogen 3 times. The mixture was stirred under hydrogen for 1 hour. Then the reaction mixture was filtered and the filtrate was concentrated. The residue was purified by chiral SFC (column: daicel chiralpak AD (250 mm*30 mm, 10 um); mobile phase: [0.1% NH₃·H₂O MeOH];B %: 35%-35%, C10; 120 min) to give (R)-tert-butyl ((4-oxo-5-(tetrahydrofuran-2-yl)-3,4-dihydrophthalazin-1-yl)methyl)carbamate (13 mg, 37.6 umol, 43% yield, 95% purity) as a white solid; LCMS [M+H]⁺: 346.1 and (S)-tert-butyl ((4-oxo-5-(tetrahydrofuran-2-yl)-3,4-dihydrophthalazin-1-yl)methyl)carbamate (14 mg, 40.5 umol, 46% yield) as a white solid; LCMS [M+H]⁺: 346.2.

Step 4: To a mixture of (R)-tert-butyl ((4-oxo-5-(tetrahydrofuran-2-yl)-3,4-dihydrophthalazin-1-yl)methyl)carbamate (14.0 mg, 40.5 umol, 1.0 eq.) in acetonitrile (0.25 mL) was added hydrochloric acid dioxane (4.0 M, 0.25 mL, 28.8 eq.) in one portion at 0° C. under nitrogen. The mixture was stirred at 20° C. for 30 minutes. The reaction mixture was concentrated. The residue was purified by prep-HPLC (column: Phenomenex luna C18 150*25 mm*10 um; mobile phase: [water (formic acid)-Acetonitrile];B %: 0%-11%, 10 min) to give (R)-4-(aminomethyl)-8-(tetrahydrofuran-2-yl)phthalazin-1 (2H)-one, Example 27-1 (2.95 mg, 11.5 umol, 28% yield, 100% ee) as a white solid. LCMS [M+H]⁺: 246.2. ¹H NMR (400 MHz, METHANOL-d₄) δ=8.19 (d, J=7.5 Hz, 1H), 7.98 (t, J=7.9 Hz, 1H), 7.80 (d, J=8.0 Hz, 1H), 6.08-5.98 (m, 1H), 4.55 (s, 2H), 3.66-3.56 (m, 2H), 1.92-1.83 (m, 1H), 1.83-1.71 (m, 2H), 1.69-1.57 (m, 1H).

Example 21-8 was obtrained from (S)-tert-butyl ((4-oxo-5-(tetrahydrofuran-2-yl)-3,4-dihydrophthalazin-1-yl)methyl)carbamate (12.0 mg, 34.7 umol, 1.0 eq.) following the same procedure as Step 4 to give (S)-4-(aminomethyl)-8-(tetrahydrofuran-2-yl)phthalazin-1 (2H)-one (6.63 mg, 26.25 umol, 76% yield, 99.9% ee, Formic Acid salt) as a white solid. LCMS [M+H]⁺: 246.2. ¹H NMR (400 MHz, METHANOL-d₄) δ=8.19 (d, J=7.6 Hz, 1H), 7.98 (t, J=8.0 Hz, 1H), 7.80 (d, J=8.0 Hz, 1H), 6.08-5.98 (m, 1H), 4.55 (s, 2H), 3.66-3.56 (m, 2H), 1.92-1.83 (m, 1H), 1.83-1.71 (m, 2H), 1.69-1.57 (m, 1H).

### Example 21-9

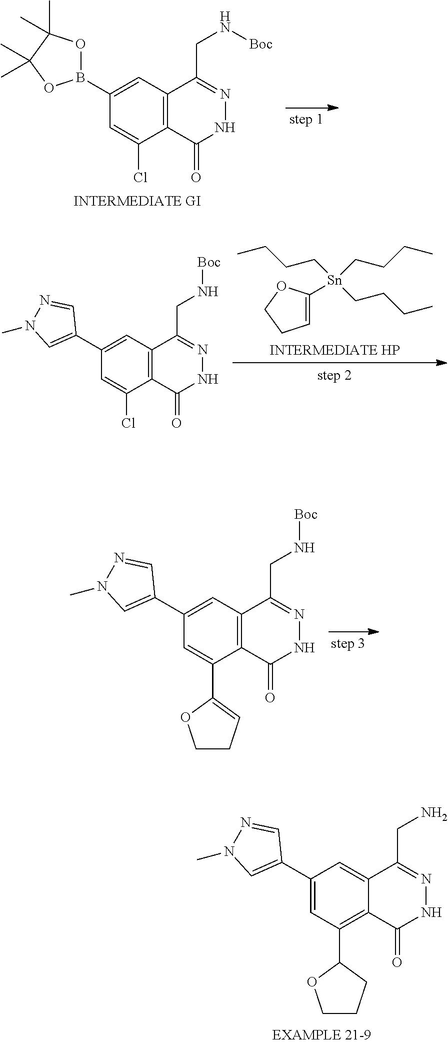

Step 1: To a mixture of INTERMEDIATE GI (260 mg, 597 μmol, 1.0 eq.), cataCXium-Pd G3 (43.5 mg, 59.7 μmol, 0.1 eq.), 4-iodo-1-methyl-pyrazole (248 mg, 1.19 mmol, 2 eq.) in tetrahydrofuran (10 mL) was added potassium phosphate (1.5 M, 995 μL, 2.5 eq.) and the mixture was stirred at 60° C. for 2 hours under nitrogen atmosphere. The reaction mixture was diluted with water (10 mL) and extracted with ethyl acetate (3×20 mL). The combined organic layers were dried over sodium sulfate, filtered and concentrated under reduced pressure. The residue was purified by column chromatography on silica (petroleum ether/ethyl acetate 10-33%), followed by reversed phase flash [water (0.1% formic acid)/acetonitrile)]. The mixture was basified to pH=8 with saturated sodium bicarbonate solution and concentrated under vacuum. The aqueous phase was extracted with ethyl acetate (20 mL). The organic layer was dried over sodium sulfate, filtered and concentrated under vacuum to give tert-butyl ((5-chloro-7-(1-methyl-1H-pyrazol-4-yl)-4-oxo-3,4-dihydrophthalazin-1-yl)methyl)carbamate (100 mg, 154 mol, 26% yield, 60% purity) as a yellow oil. LCMS [M+H]⁺: 389.9. ¹H NMR (400 MHz, dimethylsulfoxide-d₆) δ=12.45-12.42 (m, 1H), 8.48-8.42 (m, 1H), 8.19-8.04 (m, 3H), 7.48-7.41 (m, 1H), 4.47-4.37 (m, 2H), 3.89 (s, 3H), 1.41-1.37 (m, 9H).

Step 2: To a solution of INTERMEDIATE HP (18.4 mg, 51.3 μmol, 2.0 eq.) and tert-butyl ((5-chloro-7-(1-methyl-11H-pyrazol-4-yl)-4-oxo-3,4-dihydrophthalazin-1-yl)methyl)carbamate (10 mg, 25.6 μmol, 1.0 eq.) in dimethylacetamide (0.25 mL) was added cataCXium-Pd G3 (1.87 mg, 2.57 μmol, 0.1 eq.) in one portion under nitrogen. The mixture was stirred at 80° C. for 2 hours. The mixture was diluted with ethyl acetate (5 mL) and water (1 mL). The organic layer was dried over sodium sulfate, filtered and concentrated under vacuum. The mixture was purification by reversed phase flash [water (0.1% formic acid)/acetonitrile]. The mixture was basified to pH=8 with saturated sodium bicarbonate solution and concentrated under vacuum. The mixture was extracted with ethyl acetate (50 mL). The organic layer was dried over sodium sulfate, filtered and concentrated under vacuum to give tert-butyl ((5-(4,5-dihydrofuran-2-yl)-7-(1-methyl-1H-pyrazol-4-yl)-4-oxo-3,4-dihydrophthalazin-1-yl)methyl)carbamate (10.0 mg, 13.8 μmol, 54% yield, 59% purity) as a white solid. LCMS [M+H]⁺: 424.2.

Step 3: To a solution of triethylsilane (3.30 mg, 28.3 μmol, 4.53 μL, 2.0 eq.) in dichloromethane (0.6 mL) were added tert-butyl ((5-(4,5-dihydrofuran-2-yl)-7-(1-methyl-1H-pyrazol-4-yl)-4-oxo-3,4-dihydrophthalazin-1-yl)methyl)carbamate (6 mg, 14.1 μmol, 1 eq.) and boron trifluoride diethyl etherate (4.42 mg, 31.2 μmol, 3.85 μL, 2.2 eq.) under nitrogen atmosphere. The reaction mixture was stirred at 20° C. for 1 hour. The mixture was concentrated under vacuum. The residue was purified by prep-HPLC [Phenomenex luna C18 150×25 mm×10 μm; A: water (formic acid), B: acetonitrile, B %: 0/6-30% over 10 min] to give 4-(aminomethyl)-6-(1-methyl-1H-pyrazol-4-yl)-8-(tetrahydrofuran-2-yl)phthalazin-1 (2H)-one (0.05 mg, 0.15 μmol, 1% yield) as a white solid. LCMS [M+H]⁺: 326.0.

### Example 21-10

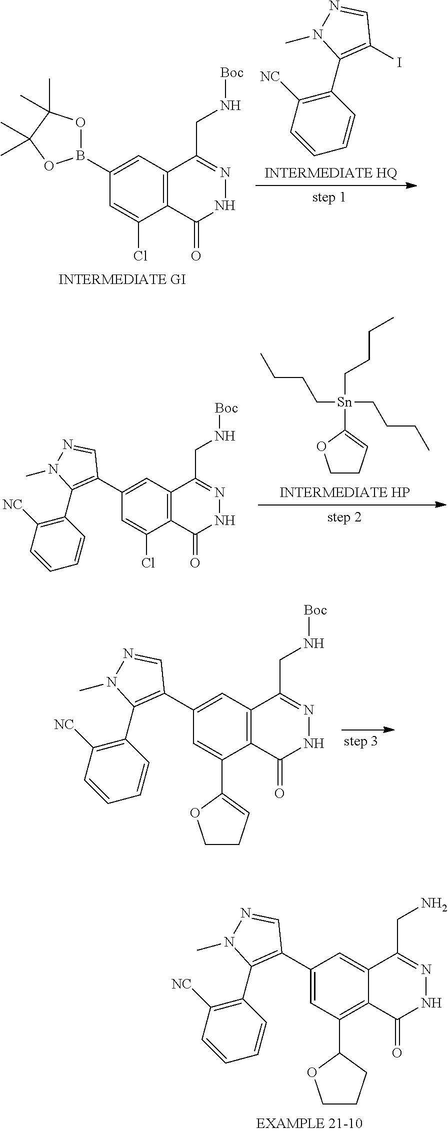 [SMILES: CC(C)(C)OC(=O)NCc1n[nH]c(=O)c2c(Cl)cc(B3OC(C)(C)C(C)(C)O3)cc12 ; Cn1ncc(I)c1-c1ccccc1C#N ; Cn1ncc(-c2cc(Cl)c3c(=O)[nH]nc(CNC(=O)OC(C)(C)C)c3c2)c1-c1ccccc1C#N ; CCC[CH2][Sn]([CH2]CCC)([CH2]CCC)[C]1=CCCO1 ; Cn1ncc(-c2cc(C3=CCCO3)c3c(=O)[nH]nc(CNC(=O)OC(C)(C)C)c3c2)c1-c1ccccc1C#N ; Cn1ncc(-c2cc(C3CCCO3)c3c(=O)[nH]nc(CN)c3c2)c1-c1ccccc1C#N]

Step 1: A mixture of Intermediate GI (170 mg, 390 μmol, 1.0 eq.), Intermediate HQ (241 mg, 780 μmol, 2.0 eq.), cataCXium Pd G3 (28.4 mg, 39.0 μmol, 0.1 eq.) and potassium phosphate (1.5 M, 650 IL, 2.5 eq.) in tetrahydrofuran (5 mL) was degassed stirred at 60° C. for 2 hours under nitrogen atmosphere. The mixture was diluted with ethyl acetate (50 mL) and water (20 mL). The organic layer was separated and dried over sodium sulfate, filtered and concentrated under vacuum. The residue was purified by column chromatography on silica gel (petroleum ether/ethyl acetate 3-30%) to give tert-butyl ((5-chloro-7-(5-(2-cyanophenyl)-1-methyl-1H-pyrazol-4-yl)-4-oxo-3,4-dihydrophthalazin-1-yl)methyl)carbamate (390 mg, 716 μmol, 92% yield, 90% purity) as a white solid. LCMS [M+H]⁺: 491.1. ¹H NMR (400 MHz, dimethylsulfoxide-d₆) δ=12.48 (s, 1H), 8.25 (s, 1H), 8.10 (br d, J=7.2 Hz, 1H), 8.01-7.94 (m, 1H), 7.87-7.75 (m, 3H), 7.33-7.22 (m, 2H), 4.18 (br d, J=4.4 Hz, 2H), 3.73 (s, 3H), 1.39 (s, 9H).

Step 2: To a solution of tert-butyl ((5-chloro-7-(5-(2-cyanophenyl)-1-methyl-1H-pyrazol-4-yl)-4-oxo-3,4-dihydrophthalazin-1-yl)methyl)carbamate (200 mg, 407 μmol, 1.0 eq.) and Intermediate HP (293 mg, 815 μmol, 2 eq.) in dimethylacetamide (5.0 mL) was added cataCXium Pd G3 (29.7 mg, 40.7 μmol, 0.1 eq.) in one portion under nitrogen. The mixture was stirred at 80° C. for 2 hours. The mixture was diluted with ethyl acetate (50 mL) and water (10 mL). The organic layer was separated dried over sodium sulfate, filtered and concentrated under vacuum. The mixture was purified by reversed phase flash [water (0.1% formic acid)/acetonitrile]. The mixture was basified to pH=8 with saturated sodium bicarbonate solution and concentrated under vacuum. The aqueous phase was extracted with ethyl acetate (50 mL). The organic layer was dried over sodium sulfate, filtered and concentrated under vacuum to give tert-butyl ((7-(5-(2-cyanophenyl)-1-methyl-1H-pyrazol-4-yl)-5-(4,5-dihydrofuran-2-yl)-4-oxo-3,4-dihydrophthalazin-1-yl)methyl)carbamate (49 mg, 80.2 μmol, 20% yield) as a white solid. LCMS [M+H]⁺: 525.1. ¹H NMR (400 MHz, dimethylsulfoxide-d₆) δ=12.62 (s, 1H), 8.20 (s, 1H), 8.06 (d, J=7.2 Hz, 1H), 7.98-7.86 (m, 2H), 7.81-7.76 (m, 2H), 7.35-7.22 (m, 1H), 7.00 (s, 1H), 4.29-4.18 (m, 2H), 3.73 (s, 3H), 3.35 (q, J=6.0 Hz, 2H), 2.58 (br t, J=7.2 Hz, 2H), 1.39 (s, 9H);

Step 3: To a solution of triethylsilane (17.7 mg, 152 μmol, 24.4 μL, 2.0 eq.) in dichloromethane (1.0 mL) were added tert-butyl ((7-(5-(2-cyanophenyl)-1-methyl-1H-pyrazol-4-yl)-5-(4,5-dihydrofuran-2-yl)-4-oxo-3,4-dihydrophthalazin-1-yl)methyl)carbamate (40.0 mg, 76.2 μmol, 1.0 eq.) and boron trifluoride diethyl etherate (23.8 mg, 168 μmol, 20.7 μL, 2.2 eq.) under nitrogen atmosphere. The mixture was stirred at 20° C. for 1 hour. The mixture was concentrated under vacuum. The residue was purification by prep-HPLC [Phenomenex luna C18 150×25 mm×10 μm; A: water (formic acid), B: acetonitrile, B %: 6%-36% over 10 min] to give 2-(4-(4-(aminomethyl)-1-oxo-8-(tetrahydrofuran-2-yl)-1,2-dihydrophthalazin-6-yl)-1-methyl-1H-pyrazol-5-yl)benzonitrile (2.94 mg, 6.63 μmol, 9% yield) as a white solid. LCMS [M+H]⁺: 427.3. ¹H NMR (400 MHz, methanol-d₄) δ=8.18 (s, 1H), 8.05-7.88 (m, 2H), 7.84-7.77 (m, 2H), 7.72-7.63 (m, 1H), 7.62-7.53 (m, 1H), 6.09-5.98 (m, 1H), 4.62-4.41 (m, 2H), 3.80 (s, 3H), 3.75-3.46 (m, 2H), 2.61-2.51 (m, 1H), 1.88-1.77 (m, 1H), 1.61-1.39 (m, 2H).

### Example 22-1

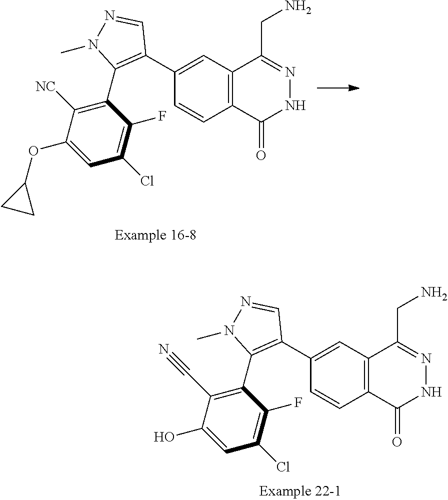

A solution of 2-[4-[4-(aminomethyl)-1-oxo-2H-phthalazin-6-yl]-2-methyl-pyrazol-3-yl]-4-chloro-6-(cyclopropoxy)-3-fluoro-benzonitrile (30.0 mg, 64.5 umol, 1.00 eq.) in trifluoromethanesulfonic acid (2.00 mL) was stirred at 15° C. for 1.5 hours. The mixture was concentrated under reduced pressure to give a residue. The residue was purified by prep-HPLC (neutral condition; column: Waters Xbridge 150*25 mm*5 um; mobile phase: [water (NH₄HCO₃)-ACN];B %: 1%-24%, 10 min) to give 2-[4-[4-(aminomethyl)-1-oxo-2H-phthalazin-6-yl]-2-methyl-pyrazol-3-yl]-4-chloro-3-fluoro-6-hydroxy-benzonitrile (8.32 mg, 19.4 umol, 30.1% yield) as a white solid. LCMS [M+1]⁺=425.0. 1H NMR (400 MHz, MeOD-d₄) δ=8.30 (d, J=8.4 Hz, 1H), 8.14 (s, 1H), 7.76-7.67 (m, 2H), 7.13 (d, J=6.24 Hz, 1H), 4.39 (s, 2H), 3.84 (s, 3H).

### Example 22-2

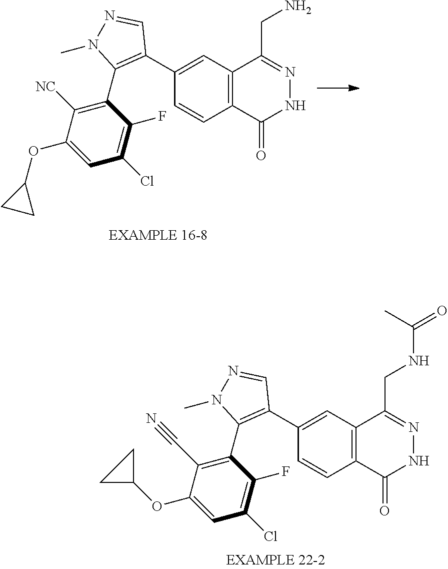

A solution of acetic acid (12.3 uL, 215 umol, 2.00 eq.), diisopropylethylamine (41.7 mg, 323 umol, 56.2 uL, 3.00 eq.) and HATU (123 mg, 323 umol, 3.00 eq.) in dimethyl formamide (2.00 mL) was stirred at 25° C. for 0.5 hour. Then 2-[4-[4-(aminomethyl)-1-oxo-2H-phthalazin-6-yl]-2-methyl-pyrazol-3-yl]-4-chloro-6-(cyclopropoxy)-3-fluoro-benzonitrile (50.0 mg, 108 umol, 1.00 eq.) was added. The mixture was stirred at 25° C. for 2 hours. The mixture was concentrated under reduced pressure to give a residue. The residue was purified by prep-HPLC (neutral condition:column: Waters Xbridge 150*25 mm*5 um; mobile phase: [water (NH₄HCO₃)-ACN];B %: 24%-54%, 9 mins) to give N-[[7-[5-[5-chloro-2-cyano-3-(cyclopropoxy)-6-fluoro-phenyl]-1-methyl-pyrazol-4-yl]-4-oxo-3H-phthalazin-1-yl]methyl]acetamide (27.9 mg, 54.9 umol, 51.0% yield, 99.6% purity) as a white solid. LCMS [M+1]⁺=507.2. 1H NMR (400 MHz, DMSO-d₆) δ=12.57 (s, 1H), 8.33 (t, J=5.6 Hz, 1H), 8.18 (s, 1H), 8.14 (d, J=8.4 Hz, 1H), 7.99 (d, J=6.4 Hz, 1H), 7.83 (s, 1H), 7.56 (dd, J=1.6, 8.4 Hz, 1H), 4.43-4.31 (m, 2H), 4.19 (tt, J=2.8, 6.0 Hz, 1H), 3.78 (s, 3H), 1.82 (s, 3H), 0.93-0.88 (m, 2H), 0.84-0.77 (m, 2H).

### Example 23-1

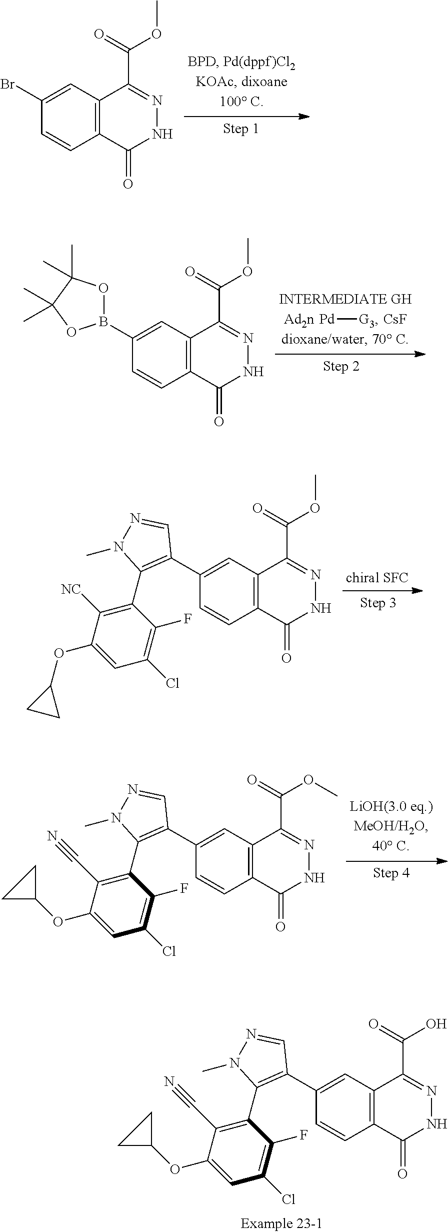

Example 23-1, (M)-7-(5-(3-chloro-6-cyano-5-cyclopropoxy-2-fluorophenyl)-1-methyl-1H-pyrazol-4-yl)-4-oxo-3,4-dihydrophthalazine-1-carboxylic acid, is prepared in 4-step sequence shown in scheme above from methyl 7-bromo-4-oxo-3H-phthalazine-1-carboxylate. In step 1 methyl 7-bromo-4-oxo-3H-phthalazine-1-carboxylate is treated with bis(pinacolato)diboron (BPD) in presence of KOAc and Pd catalyst in dioxane at 100° C. to provide methyl 4-oxo-7-(4,4,5,5-tetramethyl-1,3,2-dioxaborolan-2-yl)-3,4-dihydrophthalazine-1-carboxylate. The latter in step 2 undergoes Suzuki coupling with Intermediate GH in presence of CsF, catalytic Pd in dioxane/water at 70° C. to provide racemic methyl 7-(5-(3-chloro-6-cyano-5-cyclopropoxy-2-fluorophenyl)-1-methyl-1H-pyrazol-4-yl)-4-oxo-3,4-dihydrophthalazine-1-carboxylate which in step 3 subsequently is separated on chiral SFC to afford (M)-methyl 7-(5-(3-chloro-6-cyano-5-cyclopropoxy-2-fluorophenyl)-1-methyl-1H-pyrazol-4-yl)-4-oxo-3,4-dihydrophthalazine-1-carboxylate. Finally in step 4 the ester functionality of (M)-methyl 7-(5-(3-chloro-6-cyano-5-cyclopropoxy-2-fluorophenyl)-1-methyl-1H-pyrazol-4-yl)-4-oxo-3,4-dihydrophthalazine-1-carboxylate is hydrolyzed to acid by reacting with LiOH in MeOH/water at 40° C. to produce final compound (M)-7-(5-(3-chloro-6-cyano-5-cyclopropoxy-2-fluorophenyl)-1-methyl-1H-pyrazol-4-yl)-4-oxo-3,4-dihydrophthalazine-1-carboxylic acid, Example 23-1.

### Example A

This Example illustrates that exemplary compounds of the present invention cooperatively inhibit PRMT5 enzymatic activity in the presence of MTA.

The PRMT5 inhibitory activity of compounds of the present invention was determined using either a PRMT5:MEP50 FlashPlate Assay (Reaction Biology Corporation) or a PRMT5:MEP50 HotSpot Assay (Reaction Biology Corporation).

The assay uses purified human, PRMT5 enzyme to convert S-adenosyl-L-[methyl-³H]methionine plus histone H4 L-arginine to S-adenosyl-L-homocysteine plus histone H4 [methyl-³H]-L-arginine. The assay was carried out using Streptavidin-coated FlashPlates (Perkin Elmer), which contained a scintillant embedded in the plastic of the plate. The histone H4 peptide substrate was conjugated with biotin, which binds to the streptavidin-coated well of the plate, placing the H4 peptide in close proximity to the side well and the scintillant. The transfer of the tritiated methyl group from S-adenosyl-L-[methyl-³H]methionine to the bound histone H4 peptide generated a radiolabeled histone H4, which was quantitated by measuring in a scintillation counter to determine the activity of PRMT5 enzyme in the presence and absence of compound. The assay reactions also were conducted in the presence and absence of MTA to determine whether the compounds exhibit MTA-cooperative activity. Briefly, compounds of the present invention were solubilized in 100% DMSO at a highest concentration of 10 mM. For IC₅₀ determinations, the initial starting concentration for the serial dilutions of each compound was 50 μM. Control samples lacking compound, PRMT5/MEP50 complex or various reaction components also were prepared and processed in parallel with compound test samples. SAH was used as a positive control for assay validation. To measure PRMT5 inhibitory activity, 3 nM PRMT5/MEP50 complex (Reaction Biology Corporation) was preincubated with test compound in assay buffer containing 40 nM histone H4 peptide (amino acids 1-15)-Biotin conjugate for 20 min at room temperature. The enzymatic reaction was initiated by adding 1 μM tritiated S-adenosyl methionine (final concentration) and the reaction is allowed to proceed for 20 min. The reaction was stopped and the amount of bound, tritiated H4 peptide in each sample was determined using a scintillation counter. The IC₅₀ value for each compound was calculated from each 10-point dose-response curve for samples plus and minus MTA using GraphPad Prism software and the results for exemplary compounds of Formula (I) or Formula (II) is shown in Table A1.

TABLE A1
IC₅₀ Values for PRMT5-mediated Enzymatic Activity by
Exemplary Compounds of Formula (I) in the Presence
and Absence of MTA in the FlashPlate Assay

| | IC₅₀ + | | | IC₅₀ + | |
|---|---|---|---|---|---|
| | 2 μM | | | 2 μM | |
| | MTA | IC₅₀ | | MTA | IC₅₀ |
| Example | (nM) | (nM) | Example | (nM) | (nM) |
| 1-4 | 39000 | >100000 | 3-48 | 280 | 7980 |
| 1-5 | 1400 | 34800 | 3-49 | 110 | 3850 |
| 1-7 | 1200 | 31500 | 3-5 | 7400 | 87900 |
| 1-8 | 10000 | >100000 | 3-50 | 10 | 277 |
| 3-1 | 3700 | 89600 | 3-51 | 500 | 11100 |
| 3-10 | 100 | 3910 | 3-52 | 370 | 3000 |
| 3-11 | 5000 | 131000 | 3-53 | 28 | 1350 |
| 3-12 | 210000 | >1000000 | 3-54 | 64 | 1290 |
| 3-13 | 110 | 420 | 3-55 | 83 | 1870 |
| 3-14 | 17 | 413 | 3-56 | 550 | 11500 |
| 3-15 | 650 | 18700 | 3-57 | 30000 | 0 |
| 3-16 | 13 | 186 | 3-58 | 510 | 8070 |
| 3-17 | 770 | 15100 | 3-59 | 7.7 | 399 |
| 3-18 | 33000 | >1000000 | 3-6 | 5100 | 47100 |
| 3-19 | 180 | 6360 | 3-60 | 8100 | >100000 |
| 3-2 | 42000 | 10000 | 3-61 | 3.4 | 80 |
| 3-20 | 53000 | 0 | 3-8 | >1000000 | >1000000 |
| 3-21 | 1800 | 39400 | 3-9 | 33 | 1230 |
| 3-22 | 35000 | >100000 | 4-1 | 45000 | >100000 |
| 3-23 | 230 | 6430 | 4-10 | 1000 | >100000 |
| 3-24 | 20000 | >100000 | 4-100 | 0.5 | 80 |
| 3-25 | 71000 | >100000 | 4-101 | 0.5 | 17 |
| 3-26 | 1600 | 20600 | 4-103 | 10.5 | 115 |
| 3-27 | 4200 | 59000 | 4-104 | 0.5 | 28 |
| 3-28 | >100000 | >100000 | 4-105 | 1.8 | 71 |
| 3-29 | 10000 | 0 | 4-106 | 0.5 | 56 |
| 3-3 | 25000 | 10000 | 4-107 | 0.5 | 37 |
| 3-30 | 7700 | 85200 | 4-108 | 900 | 8650 |
| 3-31 | 80000 | >100000 | 4-109 | 120 | 4870 |
| 3-32 | 710 | 11600 | 4-11 | 110 | 2400 |
| 3-33 | 61 | 1570 | 4-110 | 1000 | >100000 |
| 3-34 | 73 | 2040 | 4-111 | 12 | 472 |
| 3-35 | 59 | 1530 | 4-12 | 7400 | >100000 |
| 3-36 | 450 | 9130 | 4-13 | 210 | 2560 |
| 3-37 | 3200 | 72100 | 4-14 | 7900 | >100000 |
| 3-38 | 370 | 4940 | 4-15 | 260 | 3870 |
| 3-39 | 920 | 24300 | 4-16 | 9.4 | 602 |
| 3-4 | 1400 | 38300 | 4-17 | 150 | 2050 |
| 3-40 | 6 | 193 | 4-18 | 280 | 4420 |
| 3-41 | 1600 | 27500 | 4-19 | 380 | >100000 |
| 3-42 | 200 | 3580 | 4-2 | 490 | 13800 |
| 3-43 | 340 | 6500 | 4-61 | 3 | 149 |
| 3-44 | 1200 | 19400 | 4-62 | 120 | 2230 |
| 3-45 | 370 | 6810 | 4-63 | 58 | 1380 |
| 4-20 | 47 | 1020 | 4-64 | 230 | 4110 |
| 4-21 | 70 | 1970 | 4-65 | 16 | 517 |
| 4-22 | 370 | 8680 | 4-67 | 4 | 204 |
| 4-23 | >100000 | >100000 | 4-68 | 10 | 243 |
| 4-24 | 840 | 13400 | 4-69 | 10 | 624 |
| 4-25 | 1700 | >100000 | 4-7 | 8400 | >100000 |
| 4-26 | 130 | 1730 | 4-70 | 10 | 221 |
| 4-27 | 1300 | >100000 | 4-71 | 4.9 | 116 |
| 4-28 | 140 | 6790 | 4-72 | 3.0 | 73 |
| 4-29 | 190 | 4030 | 4-73 | 0.6 | 24 |
| 4-3 | 37 | 926 | 4-74 | 1.1 | 76 |
| 4-30 | 400 | 4750 | 4-75 | 0.7 | 33 |
| 4-31 | 410 | 7630 | 4-76 | 1.3 | 31 |
| 4-32 | 1.6 | 45 | 4-77 | 1.1 | 96 |
| 4-33 | >100000 | >100000 | 4-78 | 0.5 | 66 |
| 4-34 | 110 | 2800 | 4-79 | 27 | 930 |
| 4-35 | 1100 | >100000 | 4-8 | 440 | >100000 |
| 4-36 | 13 | 541 | 4-80 | 58 | 1570 |
| 4-37 | 1.1 | 213 | 4-81 | 18 | 826 |
| 4-38 | 25 | 850 | 4-82 | 28 | 1340 |
| 4-39 | 1.7 | 109 | 4-83 | 14 | 344 |
| 4-4 | 230 | 5910 | 4-84 | 93 | 4040 |
| 4-40 | 10 | 276 | 4-85 | 29 | 1910 |
| 4-41 | 56 | 4810 | 4-86 | | 731 |
| 4-42 | 45 | 1380 | 4-87 | 22 | 1590 |
| 4-43 | 32 | 1520 | 4-88 | 45 | 1710 |
| 4-44 | 5.4 | 691 | 4-89 | 64 | 1220 |
| 4-45 | 16 | 291 | 4-9 | 590 | 16000 |
| 4-46 | 0.6 | 329 | 4-90 | 82 | 2730 |
| 4-47 | 19 | 667 | 4-91 | 16 | 249 |
| 4-48 | 22.0 | 750 | 4-92 | 3.5 | 137 |
| 4-49 | 300 | 5930 | 4-93 | 3.3 | 35 |
| 4-5 | 150 | 1730 | 4-94 | 2.2 | 33 |
| 4-50 | 22 | 456 | 4-95 | 4.9 | 228 |
| 4-51 | 5.8 | 530 | 4-96 | 3.9 | 116 |
| 4-52 | 18 | 669 | 4-97 | 1.6 | 49 |
| 4-53 | 55 | 1140 | 4-98 | 2.1 | 90 |
| 4-54 | 77 | 1550 | 4-99 | 15 | 547 |
| 4-55 | 1.2 | 30 | 4-158 | 2.8 | 113 |
| 4-56 | 3.8 | 107 | 4-159 | 4.0 | 68.4 |
| 4-57 | 11 | 85 | 5-1 | 72000 | >100000 |
| 4-58 | 1.8 | 36 | 5-2 | 57000 | >100000 |
| 4-59 | 2.1 | 68 | 6-1 | 5800 | >100000 |
| 4-60 | 2.3 | 117 | 7-6 | 18 | 577 |
| 6-2 | >100000 | >100000 | 8-1 | 47 | 1650 |
| 6-3 | 9400 | >100000 | 8-2 | 36 | 3150 |
| 6-4 | 19000 | 0 | 8-3 | 2.3 | 237 |
| 6-5 | >100000 | >100000 | 8-4 | 3.1 | 313 |
| 7-1 | 490 | 11200 | 8-5 | 23 | 691 |
| 7-2 | 16 | 246 | 8-6 | 52 | 2140 |
| 7-3 | 15 | 388 | 8-7 | 33 | 931 |
| 7-4 | 5.6 | 151 | 8-8 | 20 | 795 |
| 7-5 | 2.0 | 64 | 9-2 | >10,000 | N.D. |
| 3-46 | 570 | 7880 | 13-1 | 25,000 | 89,600 |
| 3-47 | 56 | 2950 | | | |

The assay uses recombinant full-length hi stone H2A as the PRMT5 substrate. Enzymatic transfer of the tritiated methyl group from S-adenosyl-L-[methyl-3H]methionine to the histone H2A protein generated a radiolabeled histone H2A4 by measuring in a scintillation counter to determine the activity of PRMT5 enzyme in the presence and absence of compound. The assay reactions also were conducted in the presence and absence of MTA to determine whether the compounds exhibit MTA-cooperative activity. Briefly, compounds of the present invention were solubilized in 100% DMSO at a highest concentration of 10 mM. For IC₅₀ determinations, the initial starting concentration for the serial dilutions of each compound was 50 μM. Control samples lacking compound, PRMT5/MEP50 complex or various reaction components also were prepared and processed in parallel with compound test samples. SAH was used as a positive control for assay validation. To measure PRMT5 inhibitory activity, 1 nM PRMT5/MEP50 complex (Reaction Biology Corporation) was preincubated with test compound in assay buffer containing 5 μM full-length histone H2A for 20 min at room temperature. The enzymatic reaction was initiated by adding 1 μM tritiated S-adenosyl methionine (final concentration) and the reaction was allowed to proceed for 60 min. The reaction was stopped and transferred to filter paper for detection. The amount of tritiated H2A in each sample was determined using a scintillation counter. The IC₅₀value for each compound was calculated from each 10-point dose-response curve for samples plus and minus MTA using GraphPad Prism software and the results for exemplary compounds of Formula (I) is shown in Tables A2 and A3.

TABLE A2
IC₅₀ Values for PRMT5-mediated Enzymatic Activity by
Exemplary Compounds of Formula (I) in the Presence
and Absence of MTA in the HotSpot Assay

| | IC₅₀ + | | | IC₅₀ + | |
|---|---|---|---|---|---|
| | 2 μM | | | 2 μM | |
| | MTA | IC₅₀ | | MTA | IC₅₀ |
| Example | (nM) | (nM) | Example | (nM) | (nM) |
| 4-112 | 1020 | — | 4-178 | 4880 | — |
| 4-113 | 561 | — | 4-179 | 1240 | — |
| 4-114 | 486 | — | 4-180 | 597 | — |
| 4-115 | 377 | — | 4-181 | 401 | — |
| 4-116 | 1080 | — | 4-182 | 1420 | — |
| 4-117 | 536 | — | 4-183 | 2770 | — |
| 4-118 | 724 | 9924 | 4-184 | 127 | — |
| 4-119 | 441 | 5260 | 4-185 | 1160 | — |
| 4-120 | >10,000 | — | 4-186 | 2310 | — |
| 4-121 | 177 | — | 4-187 | 239 | — |
| 4-122 | 3820 | — | 4-188 | 99 | — |
| 4-123 | 549 | — | 4-189 | 656 | — |
| 4-124 | 826 | — | 4-190 | 278 | — |
| 4-125 | 1000 | — | 4-191 | 612 | — |
| 4-126 | 361 | — | 4-192 | 890 | — |
| 4-127 | 2540 | — | 4-193 | >10,000 | — |
| 4-128 | 1310 | — | 4-194 | 3820 | — |
| 4-129 | 1650 | — | 4-195 | >10,000 | — |
| 4-130 | 1950 | — | 4-196 | 653 | — |
| 4-131 | 1890 | — | 4-197 | 719 | — |
| 4-132 | 2560 | — | 4-198 | 111 | — |
| 4-133 | 1570 | — | 4-199 | 276 | — |
| 4-134 | 2730 | — | 4-200 | 156 | — |
| 4-107 | 760 | — | 4-201 | 948 | — |
| 4-104 | 794 | — | 4-202 | 255 | — |
| 3-40 | 9690 | — | 4-203 | 15 | — |
| 3-59 | 1140 | >10,000 | 4-205 | 23 | — |
| 4-32 | 2040 | >10,000 | 4-206 | 35 | — |
| 4-40 | 3170 | — | 4-220 | 567 | — |
| 4-45 | 2790 | — | 4-221 | 501 | — |
| 4-36 | 2800 | — | 4-222 | 255 | — |
| 4-238 | 2310 | — | 4-242 | 102 | — |
| 4-93 | 845 | — | 4-106 | 816 | — |
| 4-96 | 868 | >10,000 | 4-105 | 1000 | — |
| 4-51 | 1510 | >10,000 | 3-50 | 3780 | >10,000 |
| 4-100 | 1510 | >10,000 | 4-16 | 8850 | — |
| 4-135 | 641 | — | 4-71 | 1050 | >10,000 |
| 4-136 | 872 | — | 4-44 | 6830 | >10,000 |
| 4-137 | 810 | — | 4-73 | 933 | — |
| 4-138 | 354 | — | 4-83 | 2710 | — |
| 4-139 | 1230 | — | 4-94 | 737 | — |
| 4-140 | 1100 | — | 4-98 | 635 | — |
| 4-141 | 794 | — | 4-101 | 892 | — |
| 4-142 | 881 | — | 8-9 | >10,000 | — |
| 4-143 | 1170 | — | 9-2 | >10,000 | — |
| 4-144 | 454 | — | 9-3 | 1273 | — |
| 4-145 | 873 | — | 9-6 | >10,000 | — |
| 4-146 | 569 | — | 9-7 | 2559 | — |
| 4-147 | 43 | 280 | 10-1 | 41.7 | — |
| 4-148 | 229 | — | 10-2 | 67.2 | — |
| 4-149 | 282 | — | 10-3 | 845 | — |
| 4-150 | 673 | — | 10-4 | 417 | — |
| 4-151 | 606 | — | 10-5 | 1150 | — |
| 4-152 | 978 | — | 10-6 | 6630 | — |
| 4-152 | 1910 | — | 10-7 | 107 | — |
| 4-153 | 1650 | — | 10-8 | 660 | — |
| 4-154 | 3280 | — | 10-9 | 1930 | — |
| 4-155 | 3460 | — | 10-10 | 2730 | — |
| 4-156 | 9130 | — | 10-11 | 34 | — |
| 4-158 | 1460 | — | 10-12 | 155 | — |
| 4-159 | 393 | — | 11-1 | 1970 | — |
| 4-160 | 1110 | — | 11-2 | 138 | — |
| 4-161 | 707 | — | 12-1 | 316 | — |
| 4-162 | 842 | — | 12-2 | 315 | — |
| 4-163 | 3820 | — | 12-3 | 212 | — |
| 4-164 | 464 | — | 12-4 | 176 | — |
| 4-165 | 2410 | — | 12-5 | 112 | — |
| 4-166 | 4510 | — | 12-6 | 730 | — |
| 4-167 | 6620 | — | 12-7 | 603 | — |
| 4-168 | 1370 | — | 12-8 | 3860 | — |
| 4-169 | 1350 | — | 12-9 | 178 | — |
| 4-170 | 728 | — | 12-11 | 234 | — |
| 4-171 | 4440 | — | 12-13 | 218 | — |
| 4-173 | 1600 | — | 12-14 | 182 | — |
| 4-174 | >10,000 | — | 14-1 | >10,000 | — |
| 4-175 | 1120 | — | 16-1 | 38 | — |
| 4-176 | 8710 | — | 16-2 | 111 | — |
| 4-177 | 5970 | — | 16-5 | 994 | — |
| | | | 16-6 | 28 | — |
| | | | 16-7 | 7070 | — |
| | | | 16-8 | 77 | — |

TABLE A3
IC₅₀ Values for PRMT5-mediated Enzymatic Activity by
Exemplary Compounds of Formula (II) or Formula (IV) in
the Presence and Absence of MTA in the HotSpot Assay

| | IC₅₀ + 2 μM |
|---|---|
| | MTA |
| Example | (nM) |
| 4-255 | 9620 |
| 4-257 | 547 |
| 4-260 | 575 |
| 4-262 | 108 |
| 4-263 | 119 |
| 4-266 | 507 |
| 4-267 | 6140 |
| 4-268 | 165 |
| 4-269 | 58.2 |
| 4-270 | 102 |
| 4-272 | 260 |
| 4-273 | 376 |
| 4-274 | 1070 |
| 4-275 | 1000 |
| 4-276 | 222 |
| 4-278 | 1001 |
| 4-288 | 1480 |
| 4-289 | 4080 |
| 4-290 | 168 |
| 4-291 | 737 |
| 9-8 | >10000 |
| 9-10 | >10000 |
| 9-11 | 1440 |
| 9-12 | 4250 |
| 10-21 | 4.09 |
| 10-22 | 86.8 |
| 10-23 | 126 |
| 10-24 | 76 |
| 12-15A | 2380 |
| 12-15B | 356 |
| 12-19 | 212 |
| 12-20 | 183 |
| 12-21 | 128 |
| 15-3 | 16.3 |
| 15-4 | 261 |
| 15-6 | 235 |
| 15-8 | 109 |
| 15-9 | 1270 |
| 16-9 | 248 |
| 16-10 | 15.8 |
| 16-11 | 102 |
| 16-12 | 26.4 |
| 16-13 | 237 |
| 16-14 | 12.3 |
| 16-15 | 95 |
| 16-16 | 13.6 |
| 16-19 | 173 |
| 16-20 | 36 |
| 16-21 | 462 |
| 16-22 | 107 |
| 16-23 | 485 |
| 16-24 | 37.2 |
| 16-25 | 964 |
| 16-26 | 8.43 |
| 16-27 | 2230 |
| 16-28 | 81 |
| 16-29 | 111 |
| 16-30 | 36.4 |
| 16-31 | 19.3 |
| 16-32 | 195 |
| 16-35 | 84 |
| 16-36 | 543 |
| 16-37 | 102 |
| 16-38 | 83.8 |
| 16-39 | 366 |
| 16-40 | 60.1 |
| 16-41 | 640 |
| 16-42 | 4.55 |
| 16-43 | 2000 |
| 16-44 | 69.5 |
| 16-45 | 1970 |
| 16-46 | 41.9 |
| 16-47 | 304 |
| 16-48 | 128 |
| 16-49 | 166 |
| 16-50 | 36.4 |
| 16-51 | 261 |
| 16-52 | 29.1 |
| 16-53 | 157 |
| 16-54 | 9.19 |
| 16-55 | 260 |
| 16-56 | 113 |
| 16-57 | 253 |
| 16-58 | 4.61 |
| 16-59 | 318 |
| 16-60 | 14.7 |
| 16-61 | 217 |
| 16-62 | 28.7 |
| 16-63 | 341 |
| 16-64 | 23.3 |
| 16-65 | 204 |
| 16-66 | 107 |
| 16-67 | 269 |
| 16-68 | 52.4 |
| 16-69 | 6540 |
| 16-70 | 542 |
| 16-71 | 142 |
| 16-72 | 113 |
| 16-73 | 909 |
| 16-74 | 346 |
| 16-75 | 5030 |
| 16-76 | 111 |
| 16-77 | 4100 |
| 16-78 | 139 |
| 16-79 | 8.25 |
| 16-80 | 415 |
| 16-81 | 42.1 |
| 16-82 | 1180 |
| 16-83 | 446 |
| 16-88 | 181 |
| 16-89 | 1360 |
| 16-90 | 248 |
| 16-91 | 454 |
| 16-92 | 2040 |
| 16-93 | 821 |
| 16-94 | 1140 |
| 16-95 | 800 |
| 16-96 | 5940 |
| 16-97 | >1000 |
| 16-98 | 552 |
| 16-99 | 9870 |
| 16-100 | 1060 |
| 16-101 | 1300 |
| 16-102 | 2090 |
| 16-107 | 634 |
| 16-108 | 5 |
| 16-109 | 1370 |
| 16-110 | 19 |
| 16-111 | 451 |
| 16-112 | 51 |
| 16-115 | 8720 |
| 16-116 | 477 |
| 16-117 | 1350 |
| 16-118 | 37 |
| 16-119 | 489 |
| 16-120 | 257 |
| 16-121 | >10,000 |
| 16-122 | 112 |
| 16-123 | 4000 |
| 16-124 | 70 |
| 16-125 | 2950 |
| 16-126 | 29 |
| 16-127 | 347 |
| 16-128 | 65 |
| 16-129 | 5250 |
| 16-130 | 92 |
| 16-131 | 347 |
| 16-132 | 251 |
| 16-133 | 1080 |
| 16-134 | 955 |
| 16-135 | 758 |
| 16-136 | 393 |
| 16-137 | >10,000 |
| 16-138 | >10,000 |
| 17-4 | 1310 |
| 17-6 | 7510 |
| 17-7 | 2310 |
| 18-1 | >10000 |
| 18-2 | 2930 |
| 19-1 | 146 |
| 19-2 | 1650 |
| 19-3 | 308 |
| 19-4 | 647 |
| 20-1 | 1270 |
| 20-2 | 200 |
| 20-3 | >10,000 |
| 20-4 | 1030 |
| 20-5 | 635 |
| 20-6 | 2250 |
| 20-7 | >10,000 |

### Example B

### Cell-Based Assays

This Example illustrates that exemplary compounds of the present invention are capable of inhibiting proliferation of MTAP-deficient cells.

1. HCT116 MTAP Knockout Model

HCT116 cells were genetically engineered to knock out the function of both MTAP alleles using a CRISPR/Cas9 system and an sgRNA targeting the MTAP gene. Following Cas9/MTAP sgRNA transduction, clones were screened to confirm that each allele of the MTAP gene had been inactivated (i.e., homozygous MTAP knock out) creating an MTAP-deficient cell. Clones confirmed to have a homozygous MTAP knockout were used in cell-based assays to assess the inhibition of PRMT5 and anti-proliferative activity and demonstrate MTA-cooperativity of compounds of the present invention.

2. Proliferation Assays

Proliferation assays were performed using HCT116 parental and HCT116 homozygous MTAP knockout cell lines to demonstrate increased potency of compounds of the present invention in the MTAP-deficient cells. Control samples were analyzed in parallel.

Briefly, on Day 0, 250 HCT116 parental or HCT116 homozygous MTAP knockout cell were seeded in 96 well plates in McCoy's 5A supplemented with 10/6 fetal bovine serum and pen/strep and the cells were incubated overnight at 37° C. plus 5% CO₂.

The following day, cells were treated with DMSO vehicle control or a dose response of PRMT5 inhibitors and incubated at 37 C plus 5% CO₂ for five days. On Day 6, the cells were trypsinized and split 1:10 into new 96-well plates with fresh medium containing the same concentration of PRMT5 inhibitor and incubated for an additional 5 days at 37° C. plus 5% CO².

On Day 11, the viability of the cells was measured using a CTG assay kit (CellTiter-Glo; Promega cat.no G7573) in accordance with the manufacturer's instructions. The IC₅₀ values for each compound after 10 days of treatment were calculated using GraphPad PRISM software.

3. SYM11 In-Cell Western

In-Cell Western assays were performed in HCT116 parental and HCT116 homozygous MTAP Knockout cell lines to demonstrate increased PRMT5 inhibition in the MTAP-deficient cells by measuring the PRMT5-dependent Symmetric Di-methyl Arginine signal. Control samples were analyzed in parallel.

Briefly, on Day 0, 2,000 HCT116 parental or HCT116 homozygous MTAP knockout cells were seeded in 96 well plates in McCoy's 5A supplemented with 10% fetal bovine serum and pen/strep and the cells were incubated overnight at 37° C. plus 5% CO₂. The following day, cells were treated with DMSO vehicle control or a dose response of PRMT5 inhibitors, and incubated at 37° C. plus 5% CO² for four days.

After 4 days of treatment, the cells were fixed by adding 50 μl of 4% paraformaldehyde solution to each well and the cells were incubated for 20 min at room temperature. The paraformaldehyde solution was removed, 150 μl of ice-cold methanol was added and the plate was placed −20° C. for 10 minutes. The methanol was removed, 150 μl of Odyssey Blocking buffer+0.05% Tween20 was added and the plate was incubated at room temperature with shaking for one hour.

To each test well, 50 μl of SYM11 antibody (Millipore 07-413) diluted 1:500 in Odyssey Blocking buffer+0.05% Tween20 was added and the plate was placed at 4° C. overnight. The primary SYM11 antibody solution was removed by aspiration and the wells were washed three consecutive times with phosphate buffered saline containing 0.1% Tween-20 (PBST).

A 50 μl aliquot of a Goat anti-Rabbit IRDye 800CW secondary antibody (Li-Cor 926-32211) diluted 1:800 and nuclear stain DRAQ5 (Biostatus Limited) diluted 1:10000 in Odyssey Blocking Buffer+DRAQ5+0.05% Tween20 was added and the plate was stored for 2 hours in dark at room temperature. The secondary antibody solution was removed by aspiration and the wells were washed three consecutive times with PBST.

The SYM11 signal and the DRAQ5 signal were quantified using a Li-Cor Odyssey machine reading at 800 nM and 700 nM, respectively. The SYM11/DRAQ5 ratio was used to calculate the inhibition of Symmetric Dimethyl Arginine as percent of DMSO control.

TABLE B
Inhibition of PRMT5-dependent Symmetric Di-methyl Arginine (SYM11) signal and
proliferation in HCT116 parental and HCT116 homozygous MTAP Knockout cell lines

| | In-cell-Western (SYM11) IC₅₀ (nM) | | Proliferation IC₅₀ (nM) | |
|---|---|---|---|---|
| Example | HCT216 parental | HCT116 MTAP_KO | HCT116 parental | HCT116 MTAP_KO |
| 4-67 | 10000 | 945 | 7700 | 2026 |
| 4-94 | 10000 | 1176 | 4877 | 1836 |
| 4-100 | 10000 | 505 | 10000 | 5230 |
| 4-101 | 6667 | 376 | — | 2309 |
| 4-103 | 6667 | 255 | — | 4391 |
| 4-104 | 3333 | 391 | — | 5324 |
| 4-105 | 10000 | 469 | — | 1395 |
| 4-106 | 3333 | 26 | — | 1596 |
| 4-107 | 10000 | 31 | — | 1057 |
| 4-127 | 5920 | 25 | >10,000 | 615 |
| 4-128 | 4024 | 39 | 9633 | 427 |
| 4-129 | >10,000 | 380 | >10,000 | 1347 |
| 4-130 | >10,000 | 24 | >10,000 | 756 |
| 4-131 | 3246 | 43 | >10,000 | 847 |
| 4-132 | >10,000 | 24 | >10,000 | 595 |
| 4-135 | 3214 | 31 | 6612 | 3764 |
| 4-137 | 5178 | 88 | >10,000 | 3236 |
| 4-139 | 5532 | 254 | 5067 | 1488 |
| 4-140 | >10,000 | 47 | 6812 | 3872 |
| 4-141 | 4958 | 88 | 5498 | 2094 |
| 4-142 | 6273 | 49 | 5737 | 1781 |
| 4-145 | 5487 | 190 | 3768 | 2161 |
| 4-146 | 6693 | 40 | >10,000 | 261 |
| 4-147 | 1260 | 11 | 1906 | 50 |
| 4-148 | 8552 | 30 | 7747 | 307 |
| 4-149 | 6351 | 135 | >10,000 | 359 |
| 4-150 | 8392 | 18 | >10,000 | 661 |
| 4-151 | 5812 | 51 | >10,000 | 504 |
| 4-158 | >10,000 | >10,000 | 1742 | 2200 |
| 4-159 | >10,000 | >10,000 | >10,000 | 8250 |
| 4-160 | >10,000 | 944 | 7700 | 2026 |
| 4-161 | 6564 | 87.5 | 6715 | 617 |
| 4-163 | >3333 | >3333 | >10,000 | >10,000 |
| 4-164 | >10,000 | 93.3 | >10,000 | 583 |
| 4-169 | 4516 | 35.5 | >10,000 | 294 |
| 4-180 | 1411 | 31.9 | 121 | 7111 |
| 4-181 | — | 25 | — | 450 |
| 4-182 | >1000 | 58 | 7252 | 572 |
| 4-183 | — | 324 | — | 897 |
| 4-184 | >10,000 | 4 | 9401 | 107 |
| 4-185 | — | 167 | — | 3022 |
| 4-186 | — | 5114 | — | 5775 |
| 4-187 | 1000 | 7 | 1363 | 1903 |
| 4-188 | >10,000 | 4 | 904 | 39 |
| 4-189 | 1833 | 31 | >10,000 | 234 |
| 4-190 | >3333 | >3333 | — | 1977 |
| 4-191 | — | 911 | — | 1519 |
| 4-192 | >10,000 | 256 | >10,000 | 2139− |
| 4-196 | 1316 | 11 | 9289 | 154 |
| 4-197 | >1000 | 25 | 8572 | 256 |
| 4-198 | 1266 | 21 | 7341 | 136 |
| 4-199 | — | 2 | 8846 | 120 |
| 4-200 | 1752 | 32 | 4297 | 71 |
| 4-201 | — | 16 | >10,000 | 155 |
| 4-202 | >1000 | 8 | 2371 | 232 |
| 4-203 | 903 | 2 | 1210 | 19 |
| 4-204 | >1000 | 12 | 1830 | 52 |
| 4-205 | >1000 | 6 | 683 | 59 |
| 4-206 | >1000 | 3 | 1003 | 34 |
| 4-207 | >10,000 | 3 | — | 98 |
| 4-208 | — | 11 | — | 124 |
| 4-209 | >1000 | 10 | 4672 | 59 |
| 4-210 | >1000 | 8 | 1365 | 416 |
| 4-211 | — | 197 | — | 2154 |
| 4-212 | — | >10,000 | — | >10,000 |
| 4-213 | — | 88 | — | 1679 |
| 4-214 | — | >10,000 | — | 4400 |
| 4-215 | >1000 | 34 | 3542 | 81 |
| 4-216 | — | 83 | — | 134 |
| 4-217 | >1000 | 87 | 2151 | 96 |
| 4-218 | — | 140 | — | 134 |
| 4-219 | — | >1000 | — | 3612 |
| 4-220 | — | 212 | — | 5158 |
| 4-221 | — | 196 | — | 2379 |
| 4-222 | — | 296 | — | 5337 |
| 4-223 | 756 | 12 | 7808 | 75 |
| 4-224 | >1000 | 10 | 7808 | 416 |
| 4-225 | >1000 | 17 | −5169 | 84 |
| 4-226 | — | 10 | — | 191 |
| 4-227 | — | >1000 | — | 2367 |
| 4-228 | — | >1000 | — | >10,000 |
| 4-229 | >1000 | 11 | 2862 | 59 |
| 4-230 | >1000 | 6 | 1886 | 41 |
| 4-231 | >1000 | 37 | 3116 | 50 |
| 4-232 | — | 148 | — | 4768 |
| 4-233 | — | >1000 | — | 4726 |
| 4-234 | — | 129 | — | 206 |
| 4-235 | — | >1000 | — | >10,000 |
| 4-236 | — | >1000 | — | >1000 |
| 4-237 | — | 136 | — | 1044 |
| 4-238 | — | 843 | — | 1942 |
| 4-239 | — | >1000 | — | >10,000 |
| 4-240 | — | >1000 | — | 3563 |
| 4-241 | >1000 | 14 | 2400 | 51 |
| 4-242 | >1000 | 2 | 4991 | 44 |
| 4-243 | — | 51 | — | 285 |
| 4-244 | — | 42 | — | 327 |
| 4-245 | — | 7 | — | 8 |
| 4-246 | — | 50 | — | 72 |
| 4-247 | — | >1000 | — | 1806 |
| 4-248 | — | >1000 | — | 1161 |
| 4-249 | — | >1000 | — | 3508 |
| 4-250 | — | 51 | — | 757 |
| 4-251 | — | 8 | — | 49 |
| 4-252 | — | 4 | — | 87 |
| 4-254 | 3333 | 377 | — | 2174 |
| 9-2 | — | >1000 | — | >10,000 |
| 9-3 | — | 141 | — | 4822 |
| 9-4 | — | >1000 | — | 4175 |
| 9-5 | >1000 | 42 | 6437 | 125 |
| 9-6 | — | >1000 | — | >10,000 |
| 9-7 | — | >1000 | — | >10,000 |
| 10-1 | 1123 | 15 | 2299 | 36 |
| 10-2 | 2484 | 6 | 3378 | 112 |
| 10-3 | 3820 | 5 | 8482 | 240 |
| 10-4 | 9215 | 7 | >10,000 | 128 |
| 10-5 | >10,000 | 30 | >10,000 | 250 |
| 10-6 | >10,000 | 219 | >10,000 | 4089 |
| 10-7 | >1000 | 31 | 8386 | 103 |
| 10-8 | 4721 | 27 | >10,000 | 276 |
| 10-9 | — | 8 | 9819 | 489 |
| 10-10 | >10,000 | 89 | >10,000 | 1254 |
| 10-11 | 4256 | 5 | 763 | 29 |
| 10-12 | >1000 | 6 | 6196 | 91 |
| 10-13 | — | 16 | — | 85 |
| 10-14 | — | 75 | — | 88 |
| 10-15 | — | 4 | — | 13 |
| 10-16 | 86 | 48 | 1762 | 86 |
| 10-17 | — | 21 | — | 21 |
| 10-19 | — | 185 | — | 312 |
| 10-20 | — | 29 | — | 35 |
| 11-1 | >1000 | 57 | >10,000 | 807 |
| 11-2 | >10,000 | 1 | 1952 | 94 |
| 12-1 | >1000 | 28 | >10,000 | 158 |
| 12-2 | 2196 | 16 | 208 | >10,000 |
| 12-3 | >1000 | 8 | 1686 | 137 |
| 12-4 | — | 436 | — | 4366 |
| 12-5 | — | 456 | — | 3172 |
| 12-6 | — | 380 | — | 7468 |
| 12-7 | — | 427 | — | 4985 |
| 12-8 | — | 2856 | — | 7796 |
| 12-9 | >10,000 | 9 | 3348 | 148 |
| 12-10 | >1,000 | 3 | 1332 | 38 |
| 12-11 | 1229 | 18 | 8705 | 125 |
| 12-12 | — | 26 | — | 240 |
| 12-13 | >1000 | 95 | 1998 | 1414 |
| 12-14 | 1806 | 15 | >10,000 | 226 |
| 12-15 | — | 189 | — | 261 |
| 12-16 | — | 0.5 | — | 75 |
| 12-15 | — | 189 | — | 261 |
| 12-16 | — | 0.4 | — | 73 |
| 12-17 | — | 10 | — | 85 |
| 12-18 | — | 10 | — | 104 |
| 13-2 | — | 64 | — | 149 |
| 13-3 | — | 298 | — | 565 |
| 15-1 | >1000 | 22 | 1557 | 51 |
| 15-2 | — | 96 | — | 211 |
| 16-1 | 316 | 2 | 757 | 15 |
| 16-2 | >1,000 | 73 | 5925 | 224 |
| 16-3 | — | 15 | — | 84 |
| 16-4 | — | 12 | — | 15 |
| 16-5 | — | 272 | — | 2850 |
| 16-6 | — | 20 | — | 64 |
| 16-7 | — | 1000 | — | 4002 |
| 16-8 | — | 0.8 | — | 24 |
| 4-255 | — | 955 | — | 1626 |
| 4-256 | — | 1000 | — | 2367 |
| 4-257 | — | — | — | 618 |
| 4-258 | — | 28 | — | 89 |
| 4-259 | — | 97 | — | 837 |
| 4-260 | — | — | — | 2680 |
| 4-261 | — | — | — | 81 |
| 4-262 | — | 98 | — | 281 |
| 4-263 | — | — | — | 132 |
| 4-264 | — | 23 | — | 83 |
| 4-265 | — | 1000 | — | 1654 |
| 4-266 | — | 324 | — | 312 |
| 4-267 | — | 1000 | — | 10000 |
| 4-268 | — | — | — | 201 |
| 4-269 | — | — | — | 182 |
| 4-270 | — | — | — | 400 |
| 4-271 | — | — | — | 289 |
| 4-272 | >1000 | 55 | >10000 | 245 |
| 4-273 | — | — | — | 1438 |
| 4-274 | — | — | — | 3378 |
| 4-275 | — | — | — | 1882 |
| 4-276 | — | — | — | 198 |
| 9-8 | — | — | — | 10000 |
| 9-9 | — | — | — | 10000 |
| 9-10 | — | — | — | 10000 |
| 9-11 | — | — | — | 2369 |
| 9-12 | — | — | — | 10000 |
| 10-21 | >1000 | 35 | — | 35 |
| 10-22 | — | 17 | — | 52 |
| 10-23 | >1000 | 15 | — | 30 |
| 10-24 | — | — | — | 166 |
| 10-25 | — | 8 | — | 30 |
| 10-26 | — | 35 | — | 83 |
| 12-19 | — | — | — | 136 |
| 12-20 | — | — | — | 269 |
| 12-21 | — | — | — | 126 |
| 15-3 | 330 | 6 | — | 22 |
| 15-4 | 551 | 551 | — | 1114 |
| 15-5 | 595 | 5 | — | 23 |
| 15-6 | — | — | — | 138 |
| 15-8 | 720 | 96 | 155 | 96 |
| 16-9 | — | 93 | — | 307 |
| 16-10 | — | 3 | — | 10 |
| 16-11 | — | — | — | 5 |
| 16-12 | — | — | — | 12 |
| 16-13 | >1000 | 232 | 770 | 211 |
| 16-14 | 378 | 3 | 4404 | 8 |
| 16-15 | — | 17 | — | 33 |
| 16-16 | — | 6 | — | 6 |
| 16-17 | — | 129 | — | 285 |
| 16-18 | — | 13 | — | 19 |
| 16-19 | — | — | — | 1071 |
| 16-20 | — | — | — | 13 |
| 16-21 | 4482 | 65 | 4214 | 112 |
| 16-22 | 1055 | 4 | 3624 | 17 |
| 16-23 | — | — | — | 538 |
| 16-24 | — | — | — | 61 |
| 16-25 | — | — | — | 664 |
| 16-26 | — | — | — | 17 |
| 16-27 | — | — | — | 321 |
| 16-28 | — | — | — | 10 |
| 16-29 | — | — | — | 292 |
| 16-30 | — | — | — | 17 |
| 16-31 | — | — | — | 16 |
| 16-32 | — | — | — | 458 |
| 16-33 | — | — | — | 626 |
| 16-34 | — | — | — | 20 |
| 16-35 | — | — | — | 5 |
| 16-37 | — | — | — | 216 |
| 16-38 | — | — | — | 39 |
| 16-39 | — | — | — | 517 |
| 16-40 | — | — | — | 12 |
| 16-41 | — | 246 | — | 290 |
| 16-42 | — | 6 | — | 7 |
| 16-43 | — | 656 | — | 1304 |
| 16-44 | — | 13 | — | 20 |
| 16-45 | — | 282 | — | 798 |
| 16-46 | — | 6 | — | 7 |
| 16-47 | — | 11 | — | 51 |
| 16-48 | — | 10 | — | 19 |
| 16-49 | — | — | — | 80 |
| 16-50 | — | — | — | 9 |
| 16-51 | — | 6 | — | 706 |
| 16-52 | — | 13 | — | 19 |
| 16-53 | — | — | — | 752 |
| 16-54 | — | — | — | 82 |
| 16-55 | — | 57 | — | 77 |
| 16-56 | — | 9 | — | 22 |
| 16-57 | — | — | — | 339 |
| 16-58 | — | — | — | 15 |
| 16-59 | — | — | — | 215 |
| 16-60 | — | — | — | 39 |
| 16-61 | — | — | — | 80 |
| 16-62 | — | — | — | 7 |
| 16-63 | — | 437 | — | 859 |
| 16-64 | — | 13 | — | 17 |
| 16-65 | — | — | — | 187 |
| 16-66 | — | — | — | 35 |
| 16-67 | — | — | — | 241 |
| 16-68 | — | — | — | 41 |
| 16-69 | — | — | — | 10158 |
| 16-70 | — | — | — | 462 |
| 16-71 | — | — | — | 50 |
| 16-72 | — | — | — | 461 |
| 16-73 | — | — | — | 1074 |
| 16-74 | — | — | — | 158 |
| 16-75 | — | — | — | 420 |
| 16-76 | — | — | — | 41 |
| 16-77 | — | — | — | 264 |
| 16-78 | — | — | — | 17 |
| 16-79 | — | — | — | 33 |
| 16-80 | — | — | — | 13 |
| 16-81 | — | — | — | 9 |
| 16-82 | — | — | — | 14 |
| 16-83 | — | — | — | 1687 |
| 16-84 | — | — | — | 123 |
| 16-85 | — | — | — | 4381 |
| 16-86 | — | — | — | 183 |
| 16-87 | — | — | — | 158 |
| 16-88 | — | — | — | 173 |
| 16-89 | — | — | — | 514 |
| 16-90 | — | — | — | 138 |
| 16-91 | — | — | — | 2666 |
| 16-92 | — | — | — | 403 |
| 16-93 | — | — | — | 3286 |
| 16-94 | — | — | — | 231 |
| 16-95 | — | — | — | 1230 |
| 16-96 | — | 22 | — | — |
| 16-97 | — | | — | 1860 |
| 16-98 | — | 25 | — | — |
| 16-99 | — | — | — | 10000 |
| 16-100 | — | — | >10000 | — |
| 16-101 | — | — | — | N/A |
| 16-102 | — | 11 | — | — |
| 16-103 | — | — | — | 4005 |
| 16-104 | — | — | — | 3448 |
| 16-106 | — | — | 1950 | 11 |
| 16-110 | 176 | 6 | 423 | 16 |
| 16-111 | 686 | 344 | 57 | 21 |
| 16-119 | — | 1000 | — | — |
| 16-120 | — | 79 | — | — |
| 17-1 | — | 538 | — | 3871 |
| 17-2 | — | 80 | — | 432 |
| 17-3 | — | — | — | 278 |
| 18-1 | — | — | — | 4135 |
| 18-2 | — | >1000 | — | 2870 |
| 19-1 | — | — | — | 587 |
| 19-2 | — | — | — | 4223 |
| 19-3 | — | — | — | 149 |
| 19-4 | — | — | — | 1030 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula IIA, IIA-1, IIB, IIB-1, IIC or IIC-1: 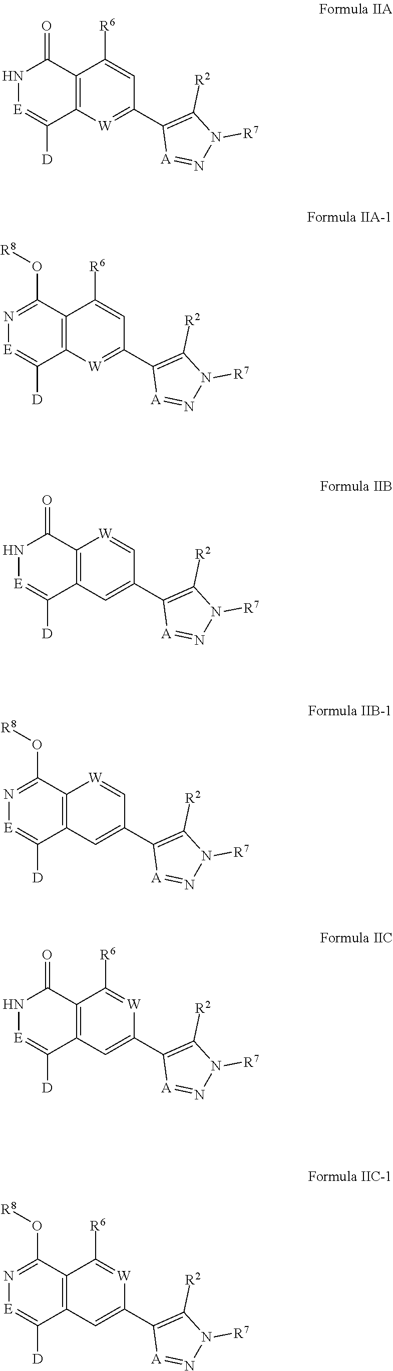
or a pharmaceutically acceptable salt thereof, wherein:
A is CR⁹ or N;
D is —CO₂R³, (C(R¹)₂)₁₋₂NHR¹¹, 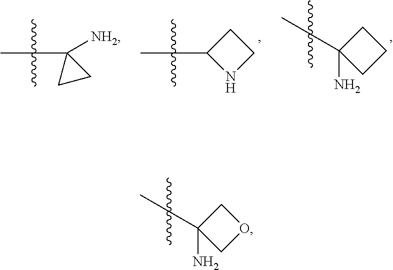 [SMILES: *C1(N)CC1 ; *C1CCN1 ; *C1(N)CCC1 ; *C1(N)COC1]
or D is 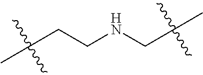 [SMILES: *CCNC*]
where the methylene is bonded to E where E is C;
E is C, CR¹ or N;
each L is independently a bond or C1-C3 alkylene;
W is CR¹⁰ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X— heteroaryl, wherein the heterocyclyl, the cycloalkyl, the aryl and the heteroaryl are optionally substituted with one or more R⁵;
each R⁴ is independently hydrogen or C1-C3 alkyl;
each R⁵ is independently cyano, oxo, halogen, pentafluorosulfanyl, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl;
each R⁹ is independently H or C1-C3 alkyl, halogen or haloalkyl;
R³⁰ is hydrogen or C1-C6 alkyl;
R¹⁰ is H, C1-C3 alkyl, halogen, haloalkyl, or C3-C7 heterocycloalkyl; and
R¹¹ represents hydrogen or —C(O)R¹², where R¹² is hydrogen or C1-C3 alkyl.

2. A compound according to claim 1, which is a compound of Formula IIA: 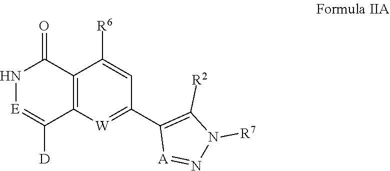
or a pharmaceutically acceptable salt thereof, wherein:
A is CR¹ or N;
D is (C(R⁹)₂)₁₋₂—NH₂, 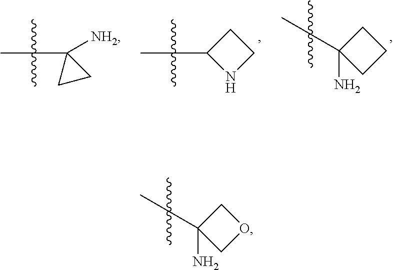 [SMILES: *C1(N)CC1 ; *C1CCN1 ; *C1(N)CCC1 ; *C1(N)COC1]
or D is 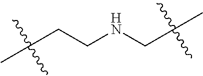 [SMILES: *CCNC*]
where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X— heteroaryl, wherein the heterocyclyl, the cycloalkyl, the aryl and the heteroaryl are optionally substituted with one or more R⁵;
each R⁴ is independently hydrogen or C1-C3 alkyl;
each R⁵ is independently cyano, oxo, halogen, pentafluorosulfanyl, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl; and
each R⁹ is independently H or C1-C3 alkyl, halogen or haloalkyl; or
a compound of Formula IIB: 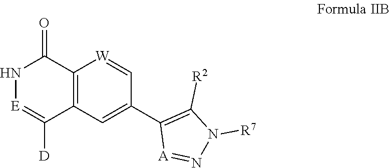
or a pharmaceutically acceptable salt thereof, wherein:
A is CR⁹ or N;
D is (C(R⁹)₂)₁₋₂—NH₂, 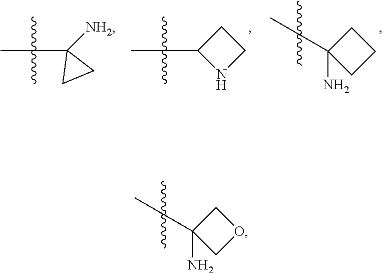 [SMILES: *C1(N)CC1 ; *C1CCN1 ; *C1(N)CCC1 ; *C1(N)COC1]
or D is 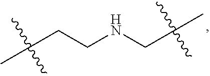 [SMILES: *CCNC*]
where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X— heteroaryl, wherein the heterocyclyl, the cycloalkyl, the aryl and the heteroaryl are optionally substituted with one or more R⁵;
each R⁴ is independently hydrogen or C1-C3 alkyl;
each R⁵ is independently cyano, oxo, halogen, pentafluorosulfanyl, C1-C3 alkyl, hydroxyalkyl, hydroxy, alkoxy, alkoxy-C1-C₃alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C₃alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl; and
each R⁹ is independently H or C1-C3 alkyl, halogen or haloalkyl; or
a compound of Formula IIC or IIC-1: 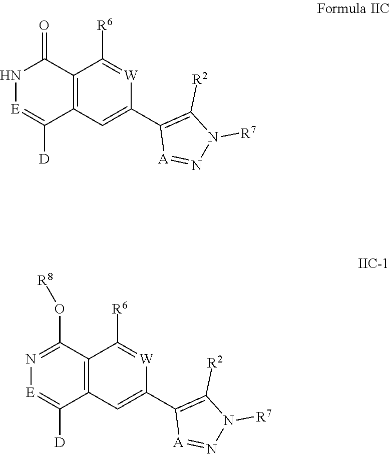
or a pharmaceutically acceptable salt thereof, wherein:
A is CR⁹ or N;
D is (C(R⁹)₂)₁₋₂—NH₂, 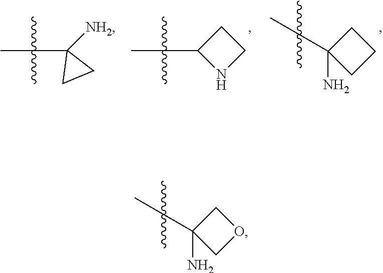 [SMILES: *C1(N)CC1 ; *C1CCN1 ; *C1(N)CCC1 ; *C1(N)COC1]
or D is 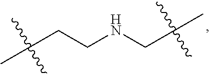 [SMILES: *CCNC*]
where the methylene is bonded to E where E is C;
E is C, CR⁹ or N;
each L is independently a bond or C1-C3 alkylene;
W is CR¹⁰ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C1-C3alkyl, —X-arC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X— heteroaryl, wherein the heterocyclyl, the cycloalkyl, the aryl and the heteroaryl are optionally substituted with one or more R⁵;
each R⁴ is independently hydrogen or C1-C3 alkyl;
each R⁵ is independently cyano, oxo, halogen, pentafluorosulfanyl, C1-C3 alkyl, hydroxyalkyl, hydroxy, alkoxy, alkoxy-C1-C₃alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁶ is hydrogen, halogen, C1-C3 alkyl, haloalkyl, hydroxy, alkoxy, C1-C3 alkyl-alkoxy, N(R⁹)₂, NR⁹C(O)R⁹, C(O)R⁹, oxetane and THF;
R⁷ is H or C1-C3 alkyl optionally substituted with one or more halogen;
R⁸ is C1-C3 alkyl;
each R⁹ is independently H or C1-C3 alkyl, halogen or haloalkyl; and
R¹⁰ is H, C1-C3 alkyl, halogen, haloalkyl, or C₁-C₆ heterocycloalkyl.

3. (canceled)

4. (canceled)

5. The compound or salt of claim 1, wherein W is CR⁹.

6. (canceled)

7. The compound or salt of claim 1, wherein E is N.

8. (canceled)

9. The compound or salt of claim 1, wherein R² is selected from: benzothiophene, naphthalene, quinoline, chromane, isochromane, dihydrobenzodioxine, indolazine, tetrahydroindolazine, dihydroisobenzofuran, benzene, isoquinolinone, benzodioxone, thienopyridine, tetrahydroindolone, indolizine, dihydroindolizinone, imadazopyridinone, thienopyrimidine, thiophene, pyrrolopyrimidinone, thiazolopyridinone, dihydropyrrolizine, isoindalone and tetrahydroisoquinoline.

10. The compound or salt of claim 1, wherein each R⁵ is independently cyano, oxo, halogen, C1-C3 alkyl, hydroxy, hydroxyalkyl, alkoxy-C1-C3alkyl, —X-L-heterocyclyl optionally substituted with one or more C1-C3alkyl or oxo, —X-L-cycloalkyl optionally substituted with C1-C3 alkyl or oxo.

11. The compound or salt of claim 1, wherein R⁶ is selected from hydrogen, hydroxy, chlorine, —NHC(O)CH₃, —C(O)CF₂H, —NH₂, —CF₂, —CH₃, —O—CH₂CH₃, —CH₂—CH₂—O—CH₃, oxetane and THF.

12. The compound or salt of claim 1, where one of L, X and Z is a bond.

13. The compound or salt of claim 12, wherein all of L, X and Z are bonds.

14. A compound of the formula (IIIA): 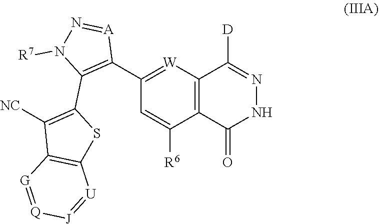
or a pharmaceutically acceptable salt thereof, wherein
A is CR⁹ or N;
D is —CH₂—NH₂, 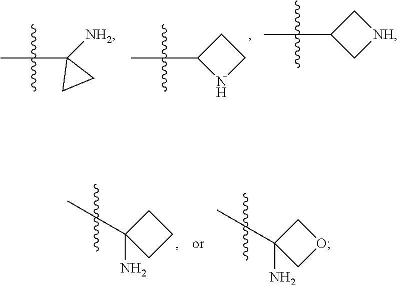 [SMILES: *C1(N)CC1 ; *C1CCN1 ; *C1CNC1 ; *C1(N)CCC1 ; *C1(N)COC1]
W is CR¹ or N, where R⁹ is H or C₁-C₃ alkyl;
G, Q, J and U are independently selected from C(H), C(R⁵), and N, provided only one or two of G, Q, J, and U can be N;
each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R′)₂, or —NR¹⁵(CO)R¹⁶, where each R¹ is independently H or C₁-C₃ alkyl, R¹⁵ is hydrogen or methyl, and R¹⁶ is C₁-C₃alkyl; and
R⁷ is C₁-C₃ alkyl or C₁-C₃ haloalkyl,
provided that the compound is not 2-(4-(4-(aminomethyl)-1-oxo-1,2-dihydrophthalazin-6-yl)-1-methyl-1H-pyrazol-5-yl)benzo[b]thiophene-3-carbonitrile or 2-(4-(4-(aminomethyl)-1-oxo-1,2-dihydrophthalazin-6-yl)-1-methyl-1H-pyrazol-5-yl)thieno[2,3-b]pyridine-3-carbonitrile; or
a compound of the formula (IIIB): 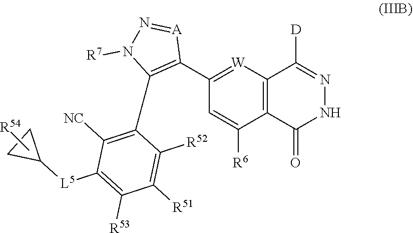
or a pharmaceutically acceptable salt thereof, wherein
A is CR⁹ or N:
D is —CH₂—NH₂, 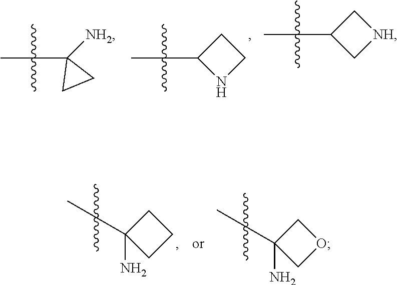 [SMILES: *C1(N)CC1 ; *C1CCN1 ; *C1CNC1 ; *C1(N)CCC1 ; *C1(N)COC1]
W is CR¹ or N, where R¹ is H or C₁-C₃ alkyl:
R⁵¹ is hydrogen, fluoro, chloro, or methyl, or R⁵¹ and R⁵² together with atoms to which they are attached form a C₄-C₆ heterocycloalkyl (e.g. hydrofuranyl):
R⁵² is fluoro, chloro, or methyl, or R⁵² and R⁵³ together with atoms to which they are attached form a phenyl;
R⁵³ is hydrogen, fluoro, chloro, or methyl:
R⁵⁴ is hydrogen, halogen, C₁-C₃ alkyl, or C₁-C₃ alkoxy:
L⁵ is —O— or —CH—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵ (CO)R¹⁶, where R¹⁵ is hydrogen or methyl, and R¹⁶ is C₁-C₃ alkyl;
R⁷ is C₁-C₃ alkyl or C₁-C₃ haloalkyl,
provided that the compound is not 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, 2-(4-(4-(aminomethyl)-1-oxo-1,2-dihydrophthalazin-6-yl)-1-methyl-1H-pyrazol-5-yl)-6-cyclopropoxy-3-fluoro-4-methylbenzonitrile, 2-(4-(4-(aminomethyl)-1-oxo-1,2-dihydrophthalazin-6-yl)-1-methyl-1H-pyrazol-5-yl)-3-chloro-6-cyclopropoxybenzonitrile, 2-(4-(4-(aminomethyl)-8-chloro-1-oxo-1,2-dihydrophthalazin-6-yl)-1-methyl-1H-pyrazol-5-yl)-6-cyclopropoxy-3-fluoro-4-methylbenzonitrile, or 2-(4-(4-(aminomethyl)-1-oxo-1,2-dihydrophthalazin-6-yl)-1-methyl-1H-pyrazol-5-yl)-6-cyclopropoxy-3-fluorobenzonitrile; or
a compound of the formula (IIIC): 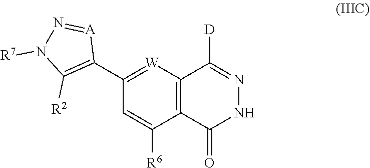
or a pharmaceutically acceptable salt thereof, wherein
A is CR⁹ or N;
D is —CH₂—NH₂, 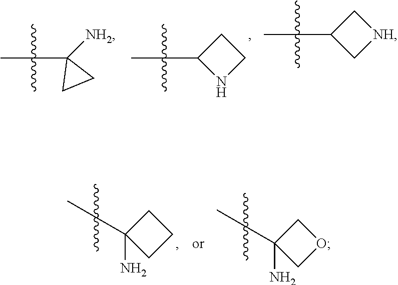 [SMILES: *C1(N)CC1 ; *C1CCN1 ; *C1CNC1 ; *C1(N)CCC1 ; *C1(N)COC1]
W is CR⁹ or N, where R⁹ is H or C₁-C₃ alkyl;
R² is 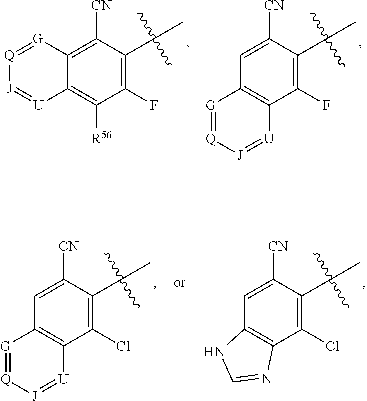
where R⁵⁶ is hydrogen, fluoro, chloro, or methyl,
G, Q, J and U are independently selected from C(H), C(R⁵), and N, provided only one or two of G, Q, J, and U can be N, and provided that when R⁵⁶ is hydrogen at least one of G, Q, J and U is C(R⁵) or N;
each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵ (CO)R¹⁶, where R¹⁵ is hydrogen or methyl, and R¹⁶ is C₁-C₃ alkyl; and
R⁷ is C₁-C₃ alkyl or C₁-C₃ haloalkyl; or
wherein
A is CR⁹ or N;
D is —CH₂—NH₂, 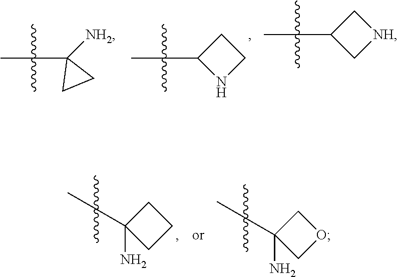 [SMILES: *C1(N)CC1 ; *C1CCN1 ; *C1CNC1 ; *C1(N)CCC1 ; *C1(N)COC1]
W is CR⁹ or N, where R⁹ is H or C₁-C₃ alkyl;
R² is 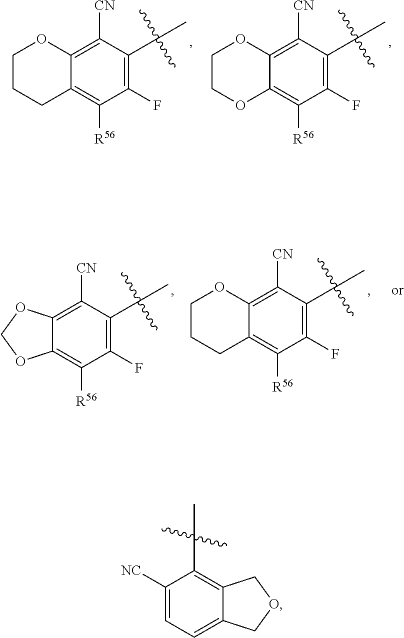
where R⁵⁶ is hydrogen, halogen, C₁-C₆ alkyl, C₁-C₆ haloalkyl, C₁-C₆ alkoxy, or C₁-C₆ haloalkoxy;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵ (CO)R¹⁶, where R¹⁵ is hydrogen or methyl, and R¹⁶ is C₁-C₃ alkyl; and
R⁷ is C₁-C₃ alkyl or C₁-C₃ haloalkyl.

15. (canceled)

16. (canceled)

17. (canceled)

18. (canceled)

19. (canceled)

20. (canceled)

21. (canceled)

22. (canceled)

23. (canceled)

24. The compound according to claim 14, which is of the formula:

25. The compound according to claim 1, which is 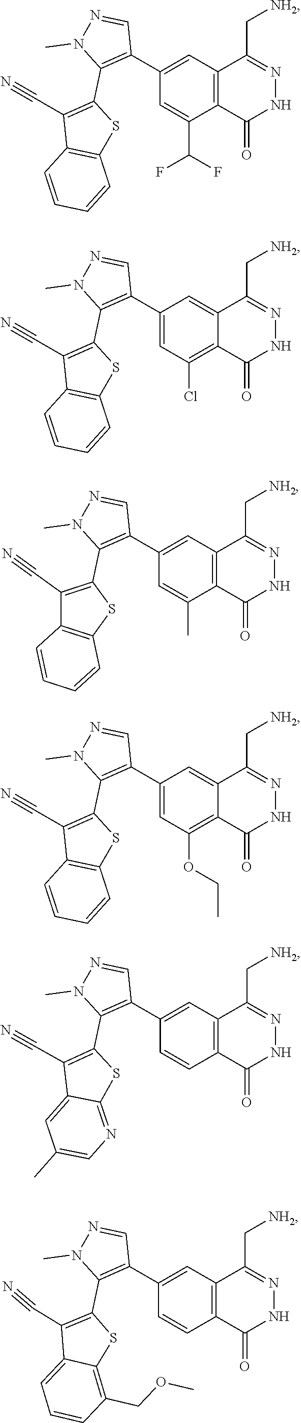 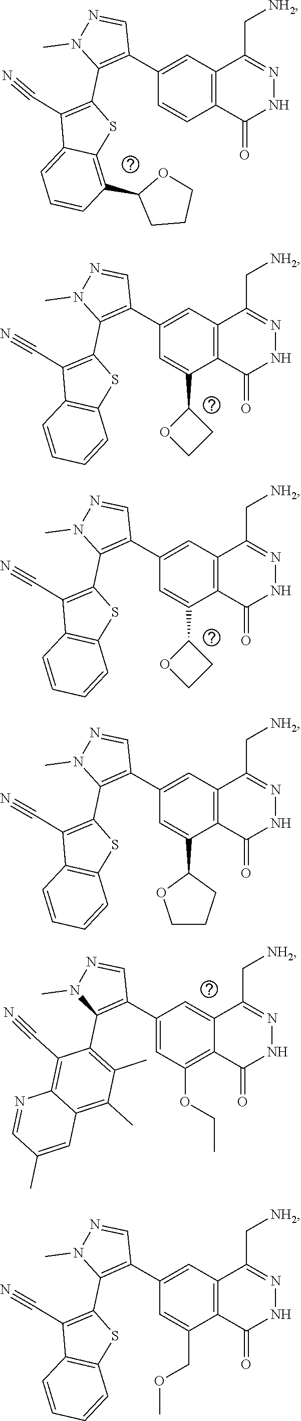 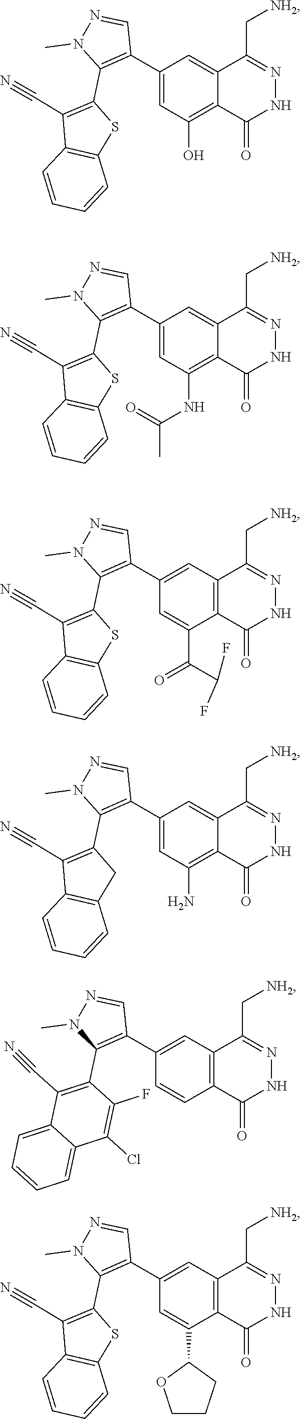 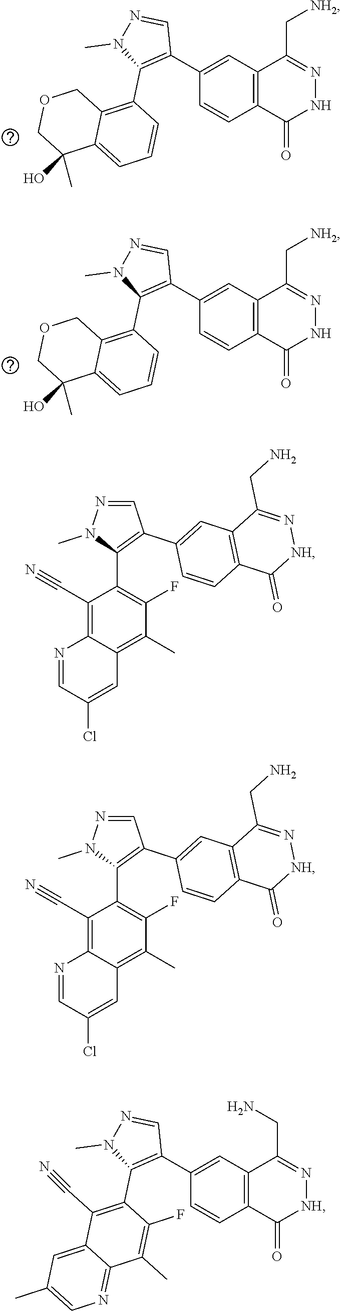 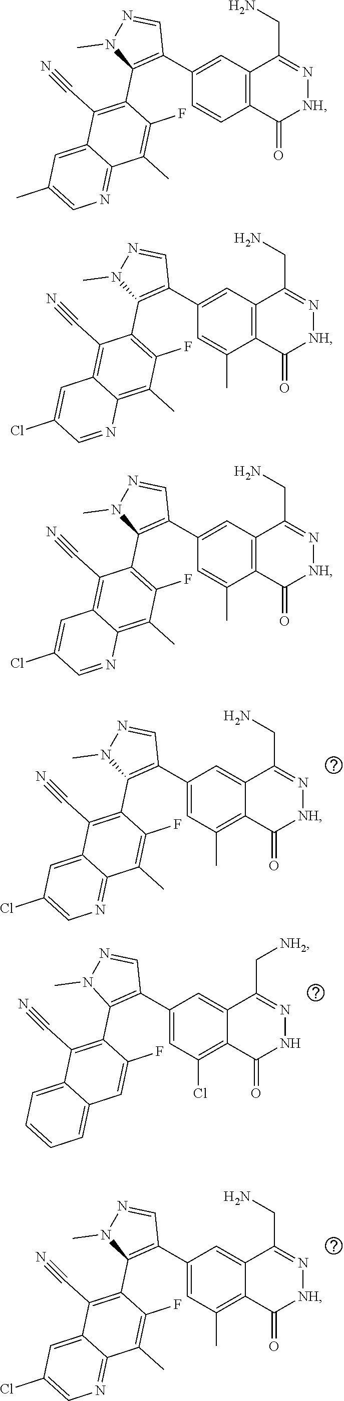 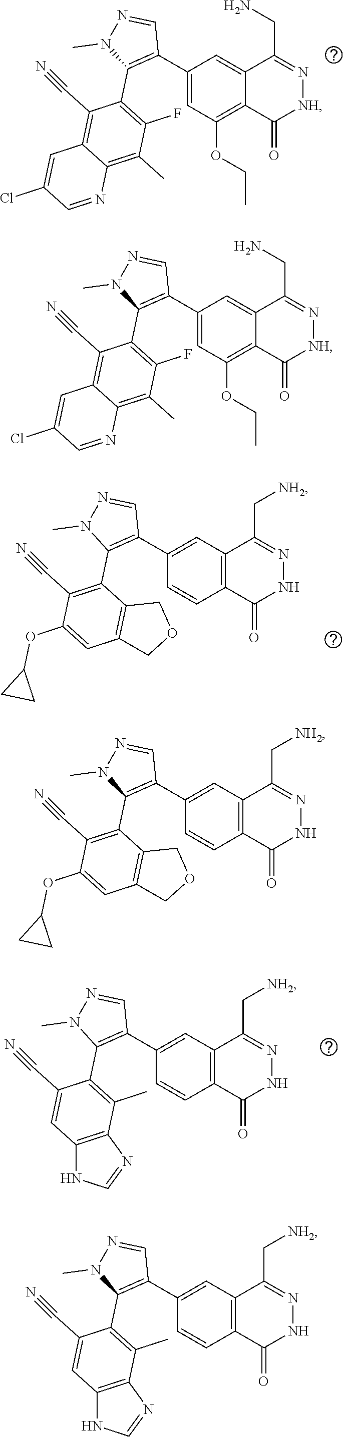 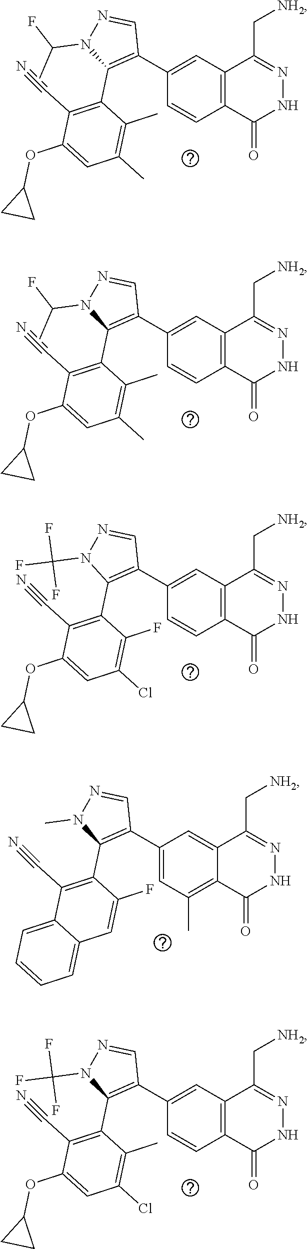 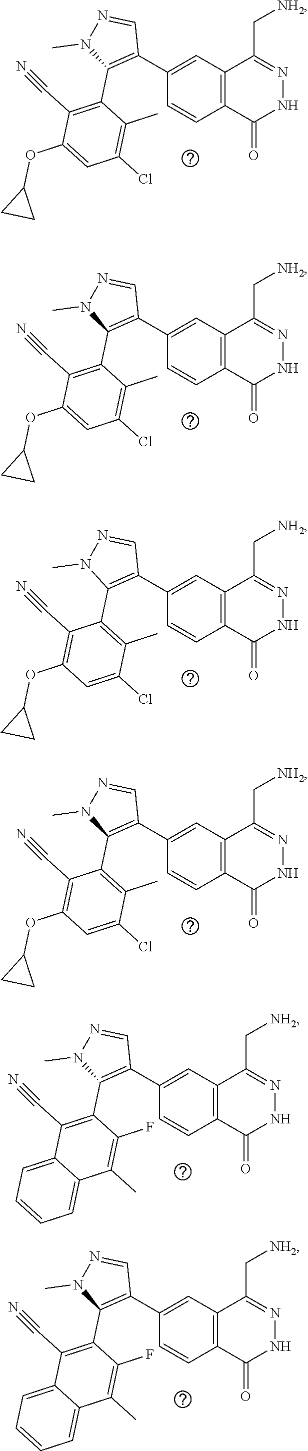 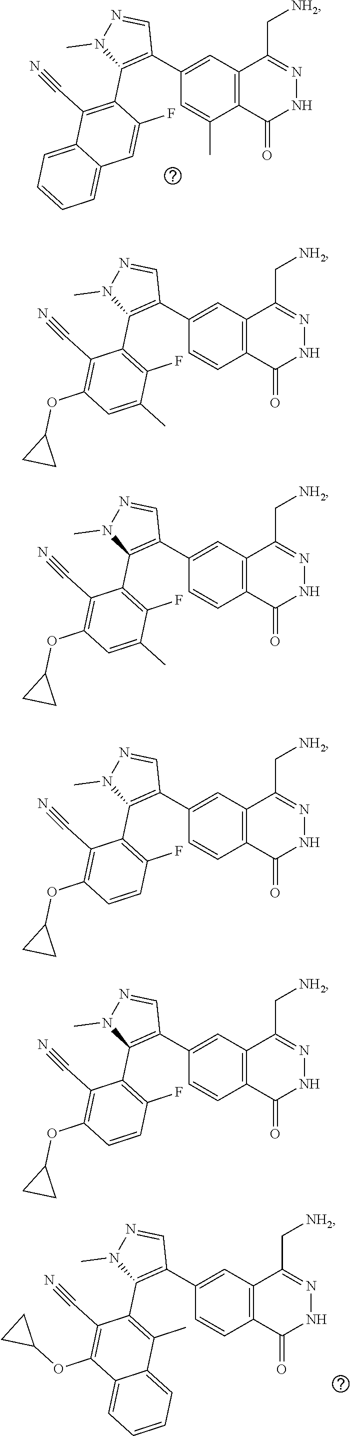 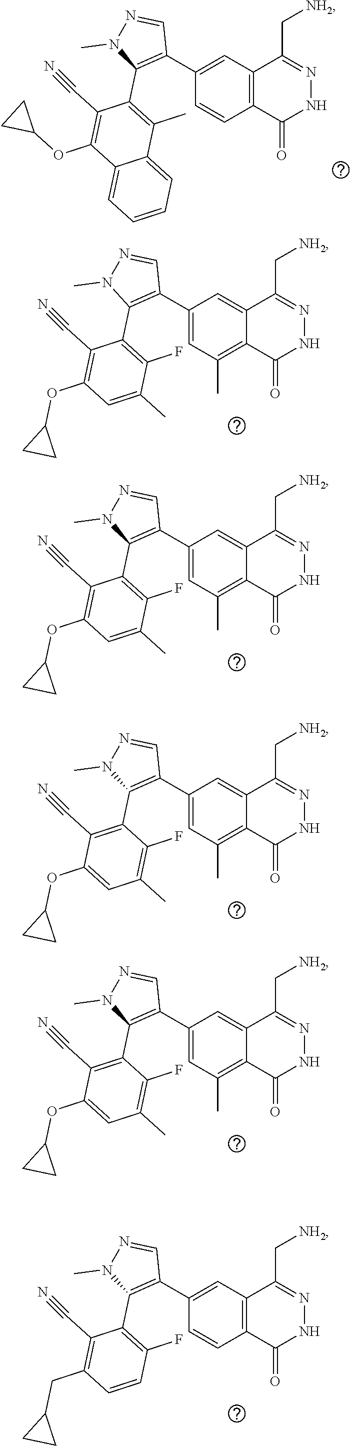 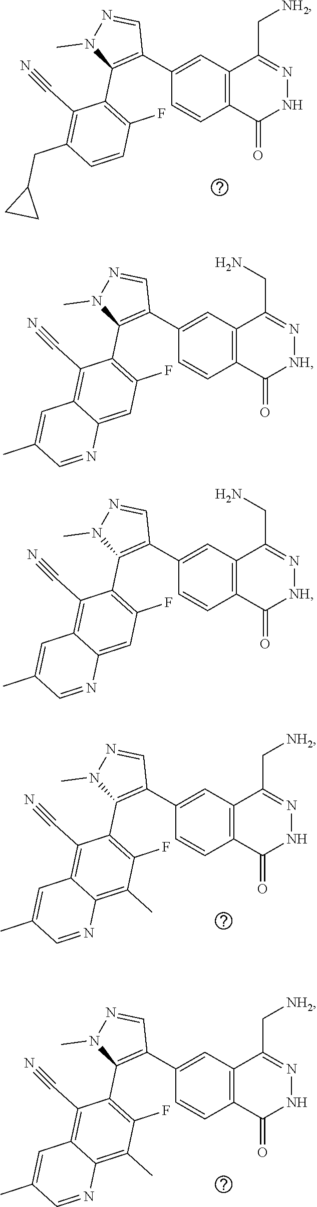 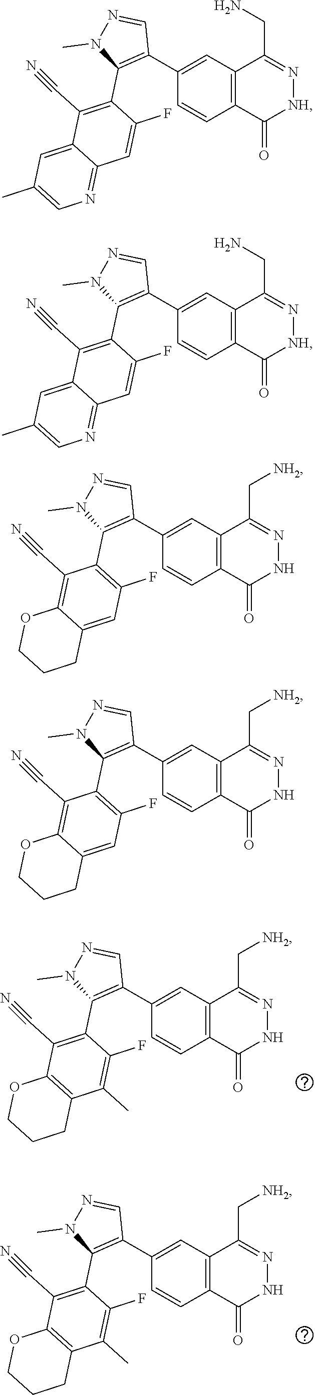 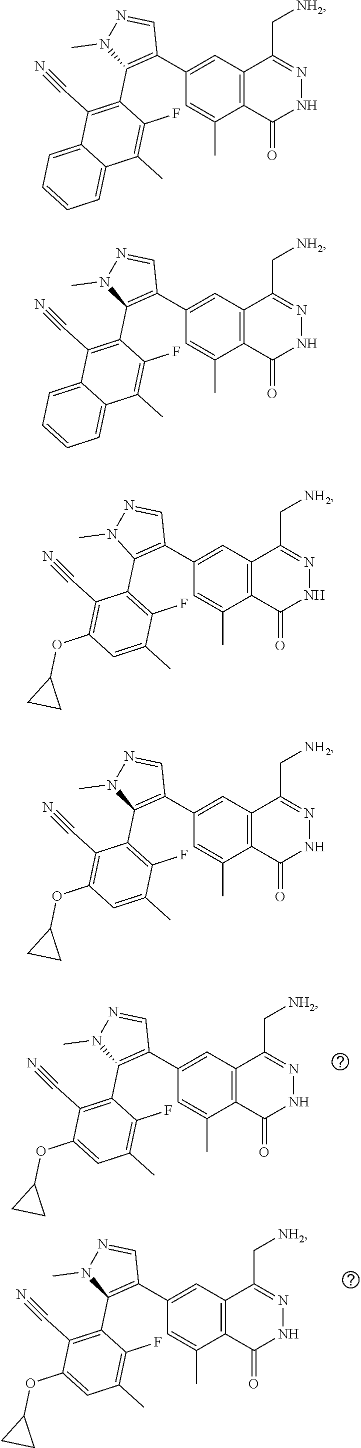 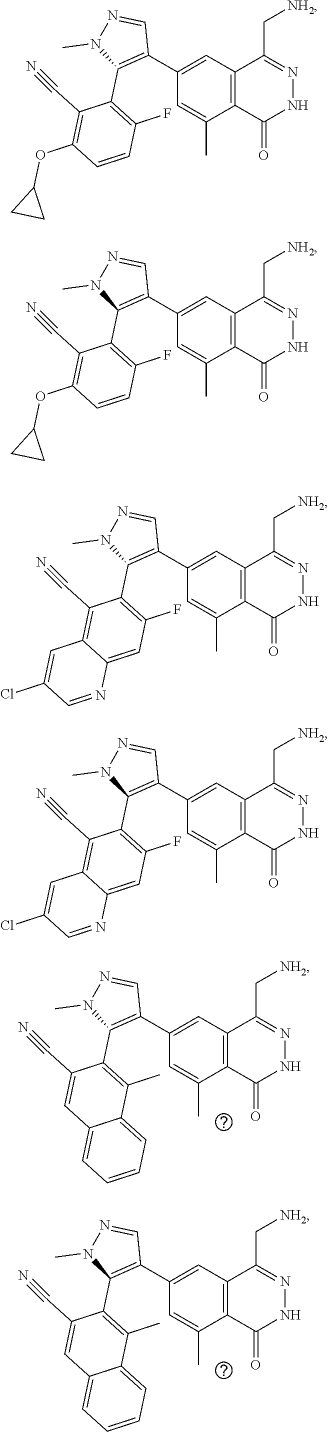 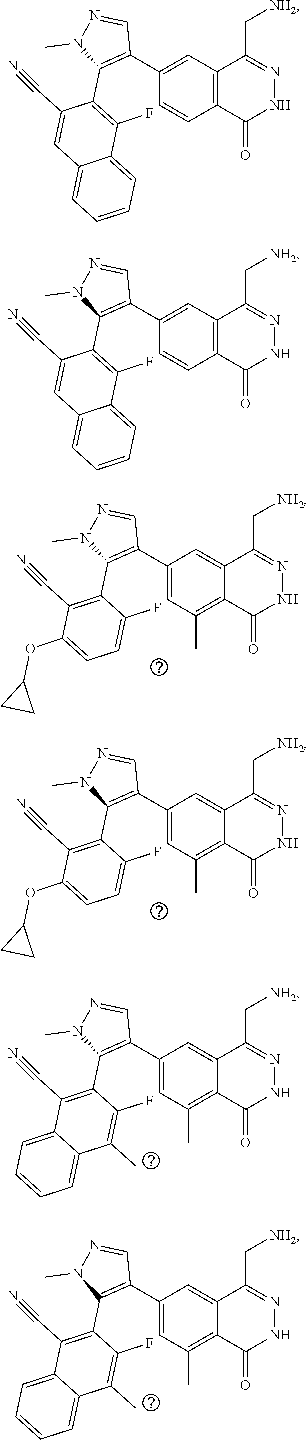 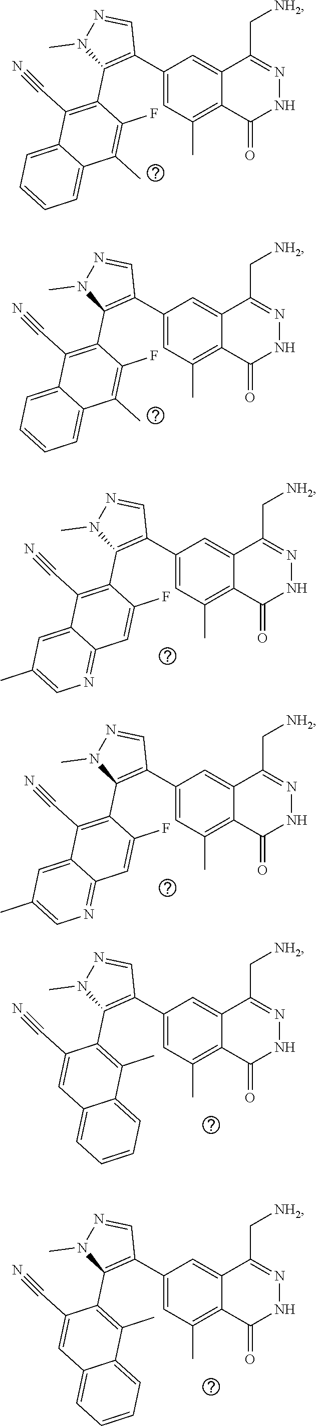 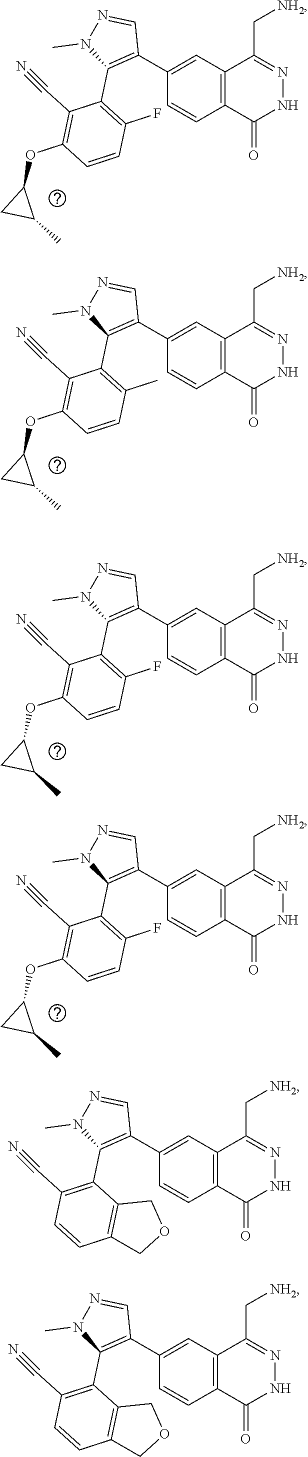 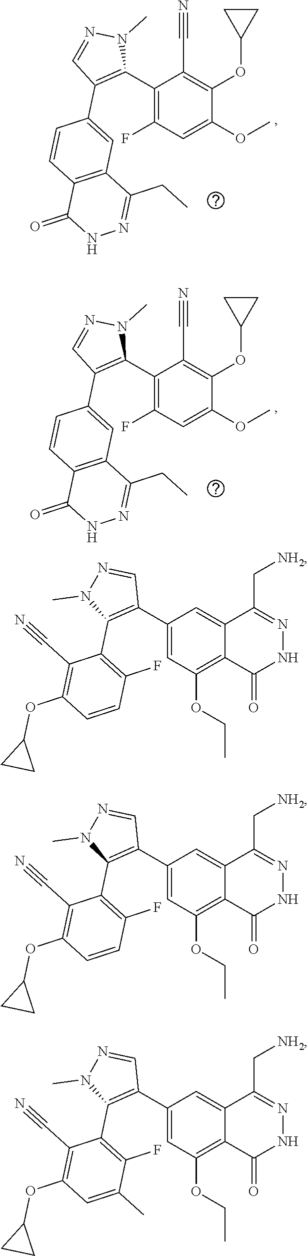 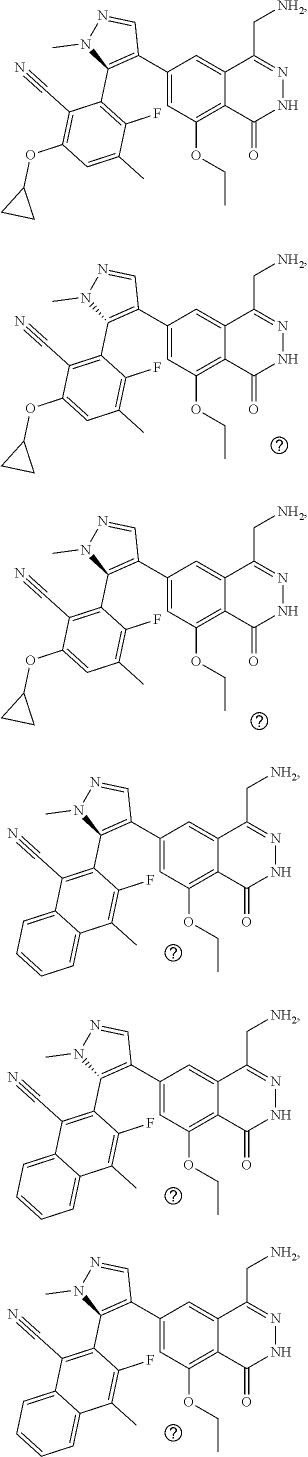 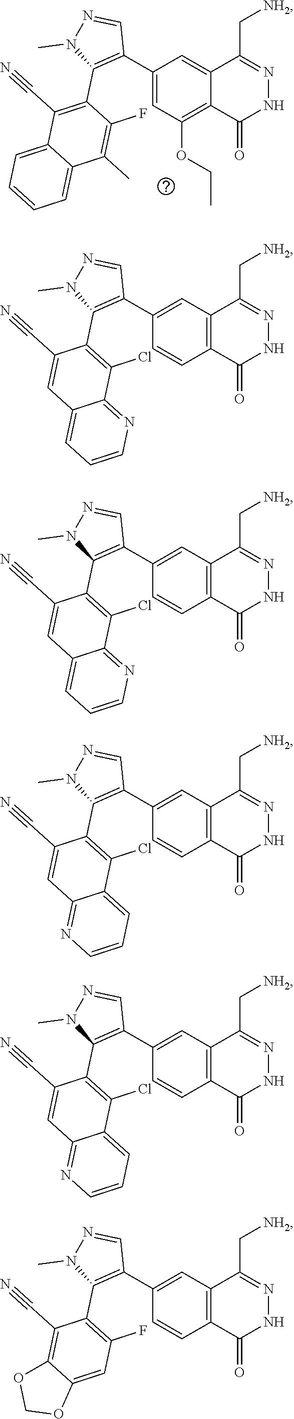 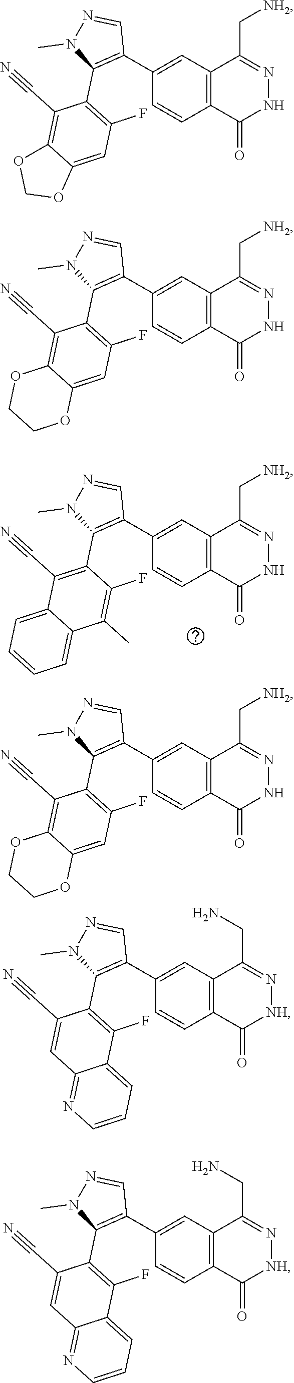 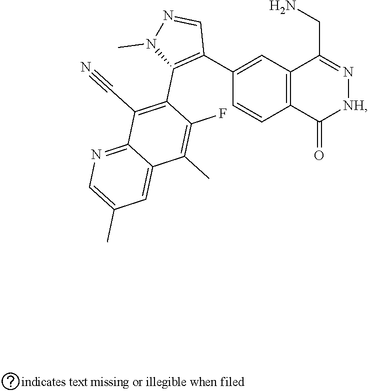 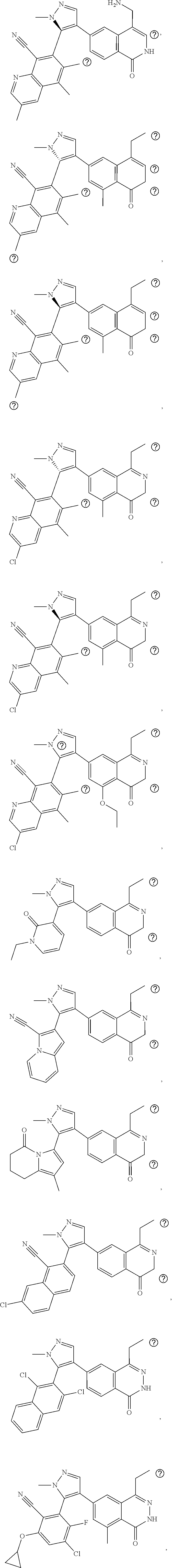 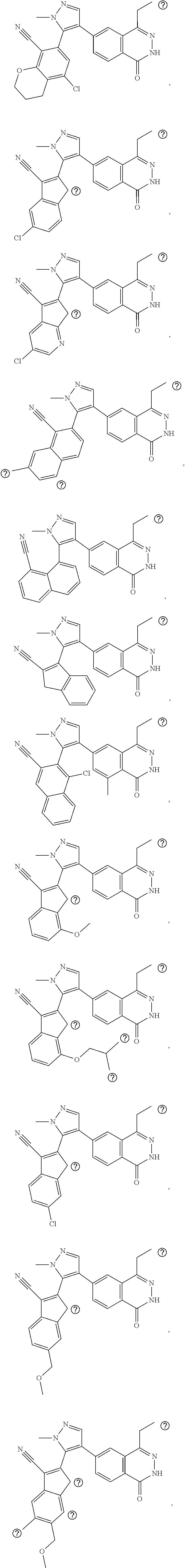 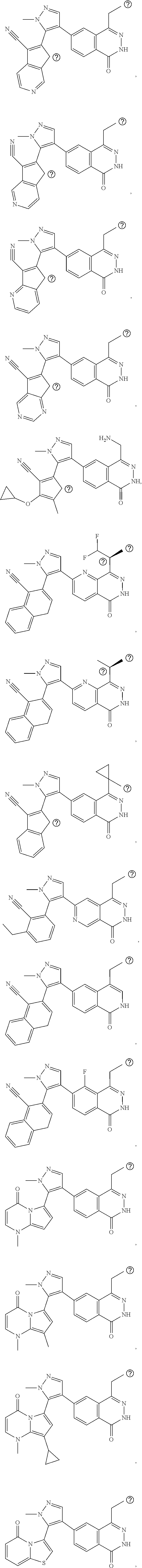 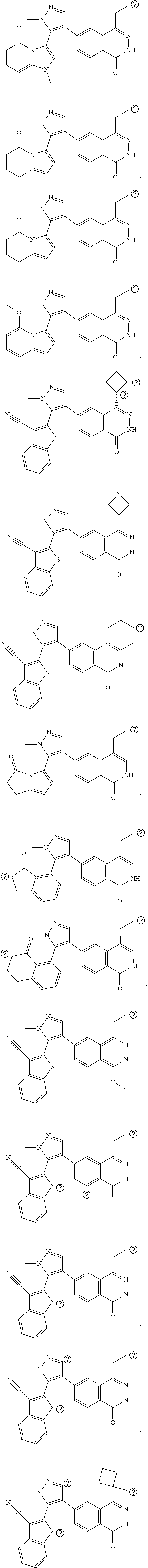 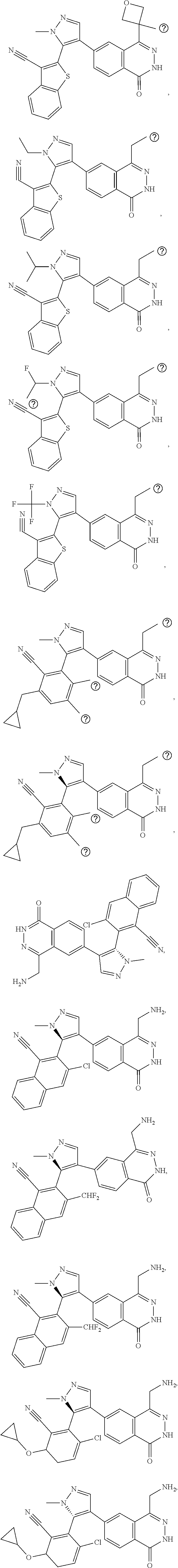 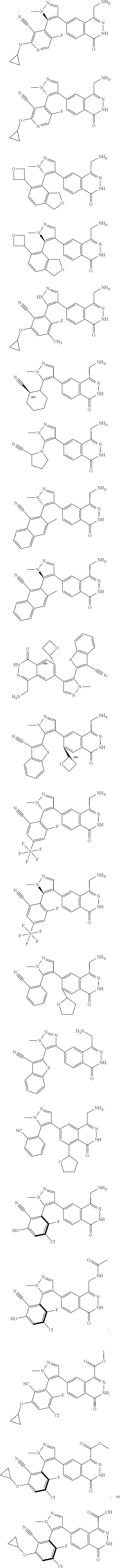
or a pharmaceutically acceptable salt thereof.

26. A compound of Formula IVA, IVA-1, IVB, IVB-1, IVC, or IVC-1: 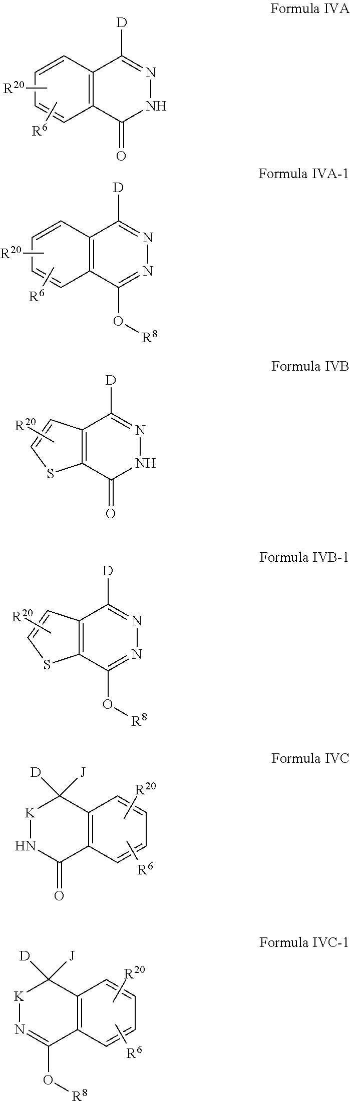
or a pharmaceutically acceptable salt thereof, wherein:
R⁶ is hydrogen, halogen, C1-C3 alkyl, haloalkyl, hydroxy, alkoxy, C1-C3 alkyl-C1-C3alkoxy, N(R⁹)₂, NR⁹C(O)R⁹, C(O)R⁹, C3-C7 heterocycloalkyl;
R² is hydrogen or a 5-membered heteroaryl group optionally substituted with one or more R²;
provided that not both R⁶ and R²⁰ are hydrogen simultaneously;
each R²² is independently hydroxy, halogen, cyano, cyanomethyl, —(NR⁴)₂, hydroxyalkyl, alkoxy, —SO₂C1-C3alkyl, —X-arylC1-C3alkyl, heteroalkyl, C2-C4 alkynyl, —X-haloalkyl, —X—C1-C5 alkyl, —Z—C1-C5 alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R²⁴;
each X is independently a bond, O, S, —NR⁴— or —NR⁴C(O)—;
each Z is independently a bond, —SO—, —SO₂—, —CH(OH)— or —C(O)—;
each L is independently a bond or C1-C3 alkylene;
each R²⁴ is independently cyano, oxo, halogen, C1-C3 alkyl, hydroxyalkyl, alkoxy, —X— haloalkyl, —Z-cycloalkyl, —X-arC1-C3alkyl, X-arC1-C3alkyl substituted with cyano, —X-L-cycloalkyl, —X-L-heteroaryl optionally substituted with one or more C1-C3alkyl or oxo, or —X-aryl;
D is —CO₂R⁰, (C(R⁹)₂)₁₋₂NHR¹¹, 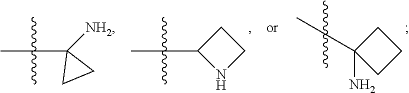 [SMILES: *C1(N)CC1 ; *C1CCN1 ; *C1(N)CCC1]
J is hydrogen or C1-C6 alkyl;
K is a bond or C(R²)₂ where each R² is independently hydrogen or C1-C3alkyl;
each R⁴ is independently hydrogen or C1-C3 alkyl;
each R² is independently cyano, oxo, halogen, pentafluorosulfanyl, C1-C3 alkyl, hydroxyalkyl, hydroxy, alkoxy, alkoxy-C1-C3alkyl, —X-haloalkyl, —Z-cycloalkyl, —X-arC1-C3alkyl, X-arC1-C3alkyl substituted with cyano, —X-L-cycloalkyl optionally substituted with C1-C3 alkyl or oxo, —X-L-heteroaryl optionally substituted with one or more C1-C3alkyl or oxo, —X-L-heterocyclyl optionally substituted with one or more C1-C3alkyl or oxo, or —X-aryl;
R⁸ is C1-C3 alkyl;
each R⁹ is independently H or C1-C3 alkyl, halogen or haloalkyl;
R³⁰ is hydrogen or C1-C6 alkyl;
R¹⁰ is H, C1-C3 alkyl, halogen, haloalkyl, or C3-C7 heterocycloalkyl; and
R¹¹ represents hydrogen or —C(O)R¹², where R¹² is hydrogen or C1-C3 alkyl.

27. A compound according to claim 26 which is 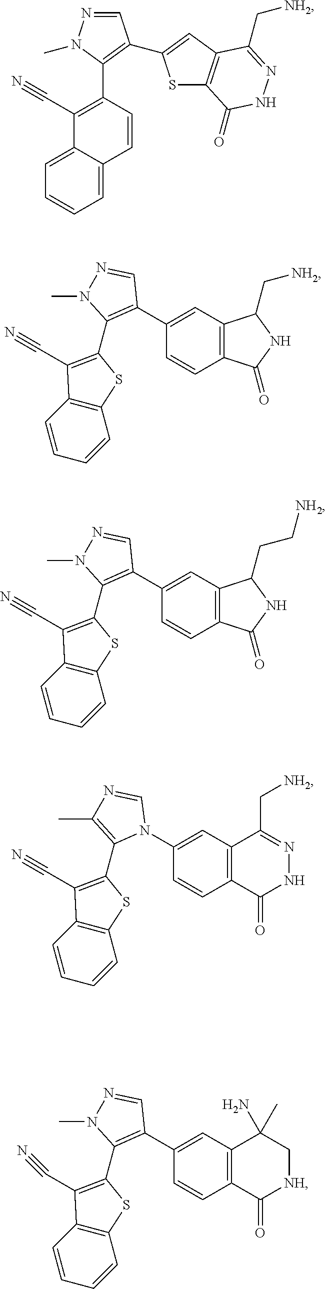 [SMILES: Cn1ncc(-c2cc3c(CN)n[nH]c(=O)c3s2)c1-c1ccc2ccccc2c1C#N ; Cn1ncc(-c2ccc3c(c2)C(CN)NC3=O)c1-c1sc2ccccc2c1C#N ; Cn1ncc(-c2ccc3c(c2)C(CCN)NC3=O)c1-c1sc2ccccc2c1C#N ; Cc1ncn(-c2ccc3c(=O)[nH]nc(CN)c3c2)c1-c1sc2ccccc2c1C#N ; Cn1ncc(-c2ccc3c(c2)C(C)(N)CNC3=O)c1-c1sc2ccccc2c1C#N] 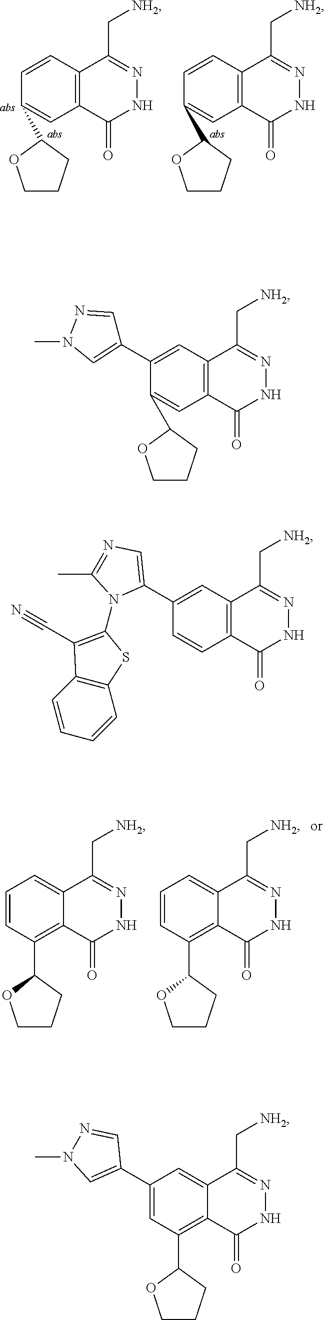
or a pharmaceutically acceptable salt thereof.

28. A pharmaceutical composition, comprising a therapeutically effective amount of a compound according to claim 1 or a pharmaceutically acceptable salt or solvate thereof, and a pharmaceutically acceptable excipient.

29. A method for inhibiting PRMT5 activity in a cell, comprising contacting the cell in which inhibition of PRMT5 activity is desired with an effective amount of a compound according to claim 1 or a pharmaceutically acceptable salt or solvate thereof, or a pharmaceutical composition comprising a therapeutically effective amount of a compound according to claim 1 or a pharmaceutically acceptable salt or solvate thereof, and a pharmaceutically acceptable excipient.

30. A method for treating cancer comprising administering to a patient having cancer a therapeutically effective amount of a compound according to claim 1 or a pharmaceutically acceptable salt or solvate thereof, alone or combined with a pharmaceutically acceptable carrier, excipient or diluents.

31. (canceled)

32. (canceled)

33. The method according to claim 30, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medukloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial wcarcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

34. The method according to claim 30, wherein the cancer is a MTAP-associated cancer.

35. The method according to claim 30, wherein the cancer is hepatocellular carcinoma, breast cancer, skin cancer, bladder cancer, liver cancer, pancreatic cancer, or head and neck cancer.
